(19) United States
(12) Patent Application Publication
(10) Pub. No.: US 2025/0269043 A1
(43) Pub. Date: Aug. 28, 2025

(54) TARGETED BIFUNCTIONAL DEGRADERS

(71) Applicant: YALE UNIVERSITY, New Haven, CT (US)

(72) Inventors: David SPIEGEL, Hamden, CT (US); David CAIANIELLO, Brooklyn, NY (US); Jake SWARTZEL, New Haven, CT (US)

(21) Appl. No.: 17/768,166

(22) PCT Filed: Oct. 9, 2020
(86) PCT No.: PCT/US20/55078

§ 371 Date: Apr. 11, 2022

Related U.S. Application Data
(60) Provisional application No. 62/913,665, filed on Oct. 10, 2019.
(60) Provisional application No. 62/913,668, filed on Oct. 10, 2019.
(60) Provisional application No. 62/913,683, filed on Oct. 10, 2019.

(51) Int. Cl. A61K 47/55; A61K 47/54; A61K 47/58; A61K 47/64; A61K 47/68; A61P 37/06

(52) U.S. Cl. CPC A61K 47/551; A61K 47/549; A61K 47/58; A61K 47/64; A61K 47/68; A61P 37/06

(57) ABSTRACT

The present disclosure provides, in one aspect, bifunctional compounds that can be used to promote or enhance degradation of certain circulating proteins. In another aspect, the present disclosure provides bifunctional compounds that can be used to promote or enhance degradation of certain autoantibodies. In certain embodiments, treatment and/or management of a disease and/or disorder requires degradation, removal, or reduction in concentration of the circulating protein or the autoantibody in the subject. Thus, in certain embodiments, administration of a compound of the disclosure to the subject removes or reduces the circulation concentration of the circulating protein or the autoantibody, thus treating, ameliorating, or preventing the disease and/or disorder. In certain embodiments, the circulating protein is TNF.

### CROSS-REFERENCE TO RELATED APPLICATIONS

This application is the U.S. national phase application filed under 35 U.S.C. § 371 claiming priority to PCT International Application No. PCT/US2020/055078, filed Oct. 9, 2020, which claims priority under 35 U.S.C. § 119(e) to U.S. Provisional Patent Applications No. 62/913,665, filed Oct. 10, 2019, No. 62/913,668, filed Oct. 10, 2019, and No. 62/913,683, filed Oct. 10, 2019, all of which are incorporated herein by reference in their entireties.

### STATEMENT REGARDING FEDERALLY SPONSORED RESEARCH OR DEVELOPMENT

This invention was made with government support under GM067543 awarded by National Institutes of Health and under W81XWH-13-1-0062 awarded by United States Army Medical Research and Material Command. The government has certain rights in the invention.

### INCORPORATION-BY-REFERENCE OF MATERIAL SUBMITTED AS A TEXT FILE VIA THE OFFICE ELECTRONIC FILING SYSTEM

This invention contains one or more sequences in a computer readable format in an accompanying text file titled “047162-7250US2-Replacement-Sequence-Listing,” created on Feb. 14, 2024, and which is 42.2 KB in size, the contents of which are incorporated herein by reference in their entirety.

### BACKGROUND OF THE DISCLOSURE

Among the mechanisms that regulate transport of molecules into a cell is receptor-mediated endocytosis. In this process, a receptor on the cell surface binds to a specific ligand (or a molecule comprising such specific ligand) that is present outside the cell—this ligand may be a small molecule, metabolite, hormone, protein, or even a virus. The binding process triggers the inward budding of the plasma membrane (invagination), forming a vesicle containing the receptor-ligand complex. The vesicle becomes an endosome and subsequently fuses with lysosomes, and the receptor is degraded along with ligand cargo bound thereto or the receptor is recycled to the cell surface for further harvesting of the circulating ligand.

One such receptor is the asialoglycoprotein receptor (ASGPR). This receptor is a C-type lectin, and its major biological role is to bind, internalize, and subsequently clear from circulation glycoproteins that contain terminal galactose or N-acetylgalactosamine residues (asialoglycoproteins). ASGPRs remove the target glycoproteins from circulation through endocytosis and subsequent lysosomal degradation. ASGPRs are highly expressed on the surface of hepatocytes, several human carcinoma cell lines, and liver cancers, and also weakly expressed by glandular cells of the gallbladder and the stomach. These receptors are known to be involved in the clearance of IgG subtypes and other antibody isotypes from circulation, removal of apoptotic cells, clearance of low density lipoprotein (LDL) and chylomicron remnants, and disposal of cellular fibronectin.

Tumor necrosis factor (TNF, also known as tumor necrosis factor alpha or TNFα) is a cell signaling protein (cytokine) involved in the acute phase systemic inflammation reaction. It is produced primarily by activated macrophages, but can be produced by other cell types such as CD4+ lymphocytes, NK cells, neutrophils, mast cells, eosinophils, and neurons. The primary role of TNF is in the regulation of immune cells. TNF is an endogenous pyrogen and can induce fever, apoptotic cell death, cachexia, and inflammation, as well as inhibit tumorigenesis and viral replication and respond to sepsis via IL1- & IL6-producing cells. Dysregulation of TNF production plays a role in diseases such as, but not limited to, Alzheimer's disease, cancer, major depression, psoriasis, and inflammatory bowel disease (IBD).

An autoantibody is an antibody that is produced by the immune system and reacts with one or more of the subject's own proteins. At times, the immune system ceases to recognize one or more of the body's normal constituents as “self,” leading to production of pathological (or disease-associated) autoantibodies. These autoantibodies proceed to attack the body's own healthy cells, tissues, or organs, causing inflammation and damage. Many autoimmune diseases, such as lupus erythematosus, are caused by such autoantibodies.

Pathological autoantibodies may target a specific organ or be systemic in nature. Autoantibodies contribute to the development and perpetuation of many diseases, such as but not limited to Guillain-Barre Syndrome, Multiple Sclerosis, Myasthenia Gravis, Atypical Hemolytic Uremic Syndrome (HUS), Catastrophic Antiphospholipid Syndrome (CAPS), Systemic Lupus Erythematosus (SLE), Chronic Inflammatory Demyelinating Polyradiculoneuropathy (CIDP), Pediatric Autoimmune Neuropsychiatric Disorders Associated with Streptococcal Infections, and Sydenham's Chorea.

Removal of disease-associated autoantibodies has been shown to attenuate symptoms and lead to improvement in clinical outcomes. Currently, strategies for reducing antibody titers include: plasmapheresis, in which the patient's plasma is separated extracorporeally from the whole blood by centrifugation/filtration and replaced by plasma from healthy donors or albumin; and intravenous immunoglobulin (IVIG), in which antibodies are pooled from donor human plasma and injected intravenously into the patient. These approaches have their limitations and drawbacks. Challenges with plasmapheresis include high cost, inconvenience, and considerable health risks and complications (such as stroke, hypotension, infection, and hypocalcemia). Similarly, IVIG has a number of drawbacks including cost, lengthy response time, and side effects (such as allergies).

There is a need in the art for novel compounds and methods that allow for inhibition, removal, and/or degradation of TNF so as to treat, ameliorate, and/or prevent certain diseases and/or disorders in a subject. There is a need in the art for novel compounds and methods that allow for inhibition, removal, and/or degradation of certain extracellular proteins so as to treat, ameliorate, and/or prevent certain diseases and/or disorders in a subject. There is a need in the art for novel compounds and methods that allow for inhibition, removal, and/or degradation of certain autoantibodies that mediate a disease and/or disorder in a subject. The present disclosure addresses these needs.

### BRIEF SUMMARY OF THE DISCLOSURE

The disclosure provides a compound comprising formula (I), or a salt, geometric isomer, stereoisomer, or solvate thereof:

[Protein binder]_{k′}-[CON]ₕ-[Linker]ᵢ-[CON]_{h′}-[CRBM]_{j′} (I),

wherein Protein binder, CON, Linker, CRBM, k′, h, i, h′, and j′ are defined elsewhere herein.

The disclosure further provides a compound comprising formula (II), or a salt, geometric isomer, stereoisomer, or solvate thereof:

[TNF binder]_{k′}-[CON]ₕ-[Linker]ᵢ-[CON]_{h′}-[CRBM]_{j′} (II),

wherein TNF binder, CON, Linker, CRBM, k′, h, i, h′, and j′ are defined elsewhere herein.

The disclosure further provides a compound comprising formula (III), or a salt, geometric isomer, stereoisomer, or solvate thereof:

[AATM]_{k′}-[CON]ₕ-[Linker]ᵢ-[CON]_{h′}-[CRBM]_{j′} (III),

wherein AATM, CON, Linker, CRBM, k′, h, i, h′, and j′ are defined elsewhere herein.

The present disclosure further provides a pharmaceutical composition comprising at least one compound contemplated herein and at least one pharmaceutically acceptable excipient.

The present disclosure further provides a method of treating a disease or disorder in a subject, the method comprising administering a therapeutically effective amount of at least one compound contemplated herein.

### BRIEF DESCRIPTION OF THE FIGURES

The drawings illustrate generally, by way of example, but not by way of limitation, various embodiments of the present application.

As used herein, the term “REAG” refers to any reagent comprising —CON, -Linker, -CON-Linker, -Linker-CON, -CON-Linker-CON, -CRBM, -CON-CRBM, -Linker-CRBM, -CON-Linker-CRBM, -Linker-CON-CRBM, and/or -CON-Linker-CON-CRBM. In certain embodiments, the REAG reacts with a TNF binder group so as to incorporate the TNF binder in the compound of the disclosure, or a fragment thereof, derivative thereof, or intermediate thereto. In certain embodiments, the REAG reacts with a Protein Binder group so as to incorporate the Protein Binder in the compound of the disclosure, or a fragment thereof, derivative thereof, or intermediate thereto. In certain embodiments, the REAG reacts with an AATM group so as to incorporate the AATM in the compound of the disclosure, or a fragment thereof, derivative thereof, or intermediate thereto. In certain embodiments, the symbol indicates no-limiting positions to which the REAG and/or Protein Binder and/or AATM can be covalently attached.

FIG. 1 illustrates a non-limiting preparation of a compound of the disclosure comprising a folic acid receptor binder.

FIG. 2 illustrates a non-limiting preparation of a compound of the disclosure comprising a mannose receptor binder.

FIG. 3 illustrates a non-limiting preparation of a compound of the disclosure comprising a mannose receptor binder.

FIG. 4 illustrates a non-limiting preparation of a compound of the disclosure comprising a mannose receptor binder.

FIG. 5 illustrates a non-limiting preparation of a compound of the disclosure comprising a mannose receptor binder.

FIG. 6 illustrates a non-limiting preparation of a compound of the disclosure comprising a mannose receptor binder.

FIG. 7 illustrates a non-limiting preparation of a compound of the disclosure comprising a mannose receptor binder.

FIG. 8 illustrates a non-limiting preparation of a compound of the disclosure comprising a mannose receptor binder.

FIG. 9 illustrates a non-limiting preparation of a polymeric compound comprising mannose-6-phosphate receptor binders.

FIG. 10 illustrates non-limiting examples of R¹ and/or R³ groups in ASGPRBM.

FIG. 11 illustrates non-limiting examples of R² groups in ASGPRBM.

FIG. 12 illustrated a non-limiting synthesis of a compound of the disclosure.

FIG. 13 illustrates a non-limiting synthesis of a TNF binder contemplated within the disclosure and its coupling to REAG so as to generate compounds of the disclosure.

FIG. 14 illustrated a non-limiting synthesis of a compound of the disclosure.

FIG. 15 illustrated a non-limiting synthesis of a compound of the disclosure.

FIG. 16 illustrated a non-limiting synthesis of a compound of the disclosure.

FIG. 17 illustrated a non-limiting synthesis of a compound of the disclosure.

FIG. 18 illustrates a non-limiting synthesis of a TNF binder contemplated within the disclosure and its coupling to REAG so as to generate compounds of the disclosure.

FIG. 19 illustrates a non-limiting synthesis of an intermediate useful for preparing certain compounds of the disclosure, such as but not limited to formula (2a).

FIG. 20 illustrates a non-limiting synthesis of a TNF binder contemplated within the disclosure and its coupling to REAG so as to generate compounds of the disclosure.

FIG. 21 illustrates a non-limiting synthesis of a TNF binder contemplated within the disclosure and its coupling to REAG so as to generate compounds of the disclosure.

FIG. 22 illustrates a non-limiting synthesis of a TNF binder contemplated within the disclosure and its coupling to REAG so as to generate compounds of the disclosure.

FIG. 23 illustrates a non-limiting synthesis of a TNF binder contemplated within the disclosure and its coupling to REAG so as to generate compounds of the disclosure.

FIGS. 24A-24B illustrate the non-limiting synthesis of an ASGPRBM group.

FIGS. 25A-25D illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

FIGS. 26A-26L illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

FIGS. 27A-270 illustrate the non-limiting synthesis of certain ASGPRBM groups. The example discloses the non-limiting Cbz protective group, but the synthesis can be performed using any other appropriate protective group as known by those skilled in the art. The protective group(s) in each intermediate and/or final product can be deprotected as appropriate.

FIGS. 28A-28B illustrate a non-limiting compound of the disclosure comprising a PCSK9 binder and its preparation.

FIG. 29 illustrates a non-limiting compound of the disclosure comprising a PCSK9 binder and its preparation.

FIG. 30 illustrates a non-limiting compound of the disclosure comprising a PCSK9 binder and its preparation.

FIG. 31 illustrates a non-limiting compound of the disclosure comprising a PCSK9 binder and its preparation.

FIG. 32 illustrates a non-limiting compound of the disclosure comprising a VEGF binder and its preparation.

FIG. 33 illustrates a non-limiting compound of the disclosure comprising a VEGF binder and its preparation.

FIG. 34 illustrates a non-limiting compound of the disclosure comprising a TGF-beta binder and its preparation.

FIG. 35 illustrates a non-limiting compound of the disclosure comprising a TGF-beta binder and its preparation.

FIG. 36 illustrates a non-limiting compound of the disclosure comprising a TSP-1 binder and its preparation.

FIGS. 37A-38B illustrate a non-limiting compound of the disclosure comprising a soluble uPAR binder and its preparation.

FIGS. 38A-38B illustrate a non-limiting compound of the disclosure comprising a PSMA binder and its preparation.

FIGS. 39A-39B illustrates a non-limiting compound of the disclosure comprising a IL-2 binder and its preparation.

FIGS. 40A-40B illustrate a non-limiting compound of the disclosure comprising a GP120 binder and its preparation.

FIG. 41 illustrates a non-limiting compound of the disclosure comprising a GP120 binder and its preparation.

FIG. 42 illustrates a non-limiting preparation of a compound of the disclosure comprising a MIF binder.

FIG. 43 illustrates a non-limiting preparation of a compound of the disclosure comprising a MIF binder.

FIG. 44 illustrates a non-limiting preparation of a compound of the disclosure comprising a MIF binder.

FIG. 45 illustrates a non-limiting preparation of a compound of the disclosure comprising a MIF binder.

FIG. 46 illustrates a non-limiting preparation of a compound of the disclosure comprising a MIF binder.

FIG. 47 illustrates a non-limiting preparation of a compound of the disclosure comprising a MIF binder.

FIG. 48 illustrates a non-limiting preparation of a compound of the disclosure comprising a MIF binder.

FIG. 49 illustrates a non-limiting preparation of a compound of the disclosure comprising a MIF binder.

FIG. 50 illustrates a non-limiting preparation of a compound of the disclosure comprising a MIF binder.

FIGS. 51A-51B illustrate non-limiting PCSK9 ligands and illustrative synthesis thereof.

FIGS. 52A-52B illustrate non-limiting PCSK9 ligands and illustrative synthesis thereof.

FIGS. 53A-53B illustrate non-limiting PCSK9 ligands and illustrative synthesis thereof.

FIG. 54 illustrates non-limiting PCSK9 ligands and illustrative synthesis thereof.

FIGS. 55A-55N illustrate the non-limiting synthesis of certain ASGPRBM groups and/or compounds of the disclosure, using a MIF binder as a non-limiting Protein binder. Any protective group(s) in each intermediate and/or final product can be deprotected as appropriate.

FIGS. 56A-56O illustrate the non-limiting synthesis of certain ASGPRBM groups and/or compounds of the disclosure, using a MIF binder as a non-limiting Protein binder. Any protective group(s) in each intermediate and/or final product can be deprotected as appropriate.

FIGS. 57A-57M illustrates a non-limiting synthesis of a TNF binder contemplated within the disclosure and its coupling to REAG so as to generate compounds of the disclosure, such as but not limited to formula (2b).

FIG. 58 illustrates certain compounds of formula (2b), wherein R represents R^{3b} in a non-limiting embodiment.

FIG. 59 illustrated a non-limiting synthesis of an intermediate that can be used to prepare a compound of formula (2b).

FIG. 60 illustrated a non-limiting synthesis of an intermediate that can be used to prepare a compound of formula (2b).

FIG. 61 illustrated a non-limiting synthesis of an intermediate that can be used to prepare a compound of formula (2b).

FIG. 62 illustrated a non-limiting synthesis of an intermediate that can be used to prepare a compound of formula (2b).

FIG. 63 illustrated a non-limiting synthesis of an intermediate that can be used to prepare a compound of formula (2b).

FIG. 64 illustrated a non-limiting synthesis of an intermediate that can be used to prepare a compound of formula (2b).

FIG. 65 illustrated a non-limiting synthesis of an intermediate that can be used to prepare a compound of formula (2b).

FIG. 66 illustrated a non-limiting synthesis of an intermediate that can be used to prepare a compound of formula (2b).

FIG. 67 illustrated a non-limiting synthesis of an intermediate that can be used to prepare a compound of formula (2b).

FIG. 68 illustrated a non-limiting synthesis of an intermediate that can be used to prepare a compound of formula (2b).

FIG. 69 illustrated a non-limiting synthesis of an intermediate that can be used to prepare a compound of formula (2b).

FIGS. 70A-70C illustrate non-limiting syntheses of certain intermediates that can be used to prepare a compound of formula (2b) (providing R³) or of formula (2c) (providing R²).

FIG. 71 illustrates a non-limiting synthesis of certain compounds of the disclosure, such as but not limited to formula (2c).

FIG. 72 illustrated a non-limiting synthesis of an intermediate that can be used to prepare a compound of formula (2c).

FIG. 73 illustrated a non-limiting synthesis of an intermediate that can be used to prepare a compound of formula (2c).

FIG. 74 illustrated a non-limiting synthesis of a compound of formula (2c).

FIG. 75 illustrates the structure of GalNAc-NH₂.

FIG. 76 illustrates a non-limiting synthesis of Indole-GN₃, a bifunctional molecule that targets the degradation of human IgG/IgF/IgM.

FIG. 77 illustrates a non-limiting synthesis of AMD-GN₃, a bifunctional molecule that targets the selective degradation of human IgG.

FIG. 78 illustrates a non-limiting synthesis of FcIII-GN₃, a bifunctional molecule that targets the selective degradation of human IgG.

FIGS. 79A-79B illustrate in vivo data that demonstrate cleavage of anti-DNP IgG in mouse serum mediated by DNP-GN₃. FIG. 79A: Mouse experiment showing that bifunctional molecule DNP-GN₃ can induce degradation of injected anti-DNP IgG antibodies in mouse serum while the negative control molecule or vehicle control did not show such effect. Purple arrow: Mice were injected with anti-DNP IgG antibodies i.p.; Green arrows: Mice were injected i.p. with PBS (vehicle), DNP-(OH)₃ (negative control) or DNP-GN₃. FIG. 79B: Structure of DNP-GN₃.

FIGS. 80-84 illustrate non-limiting synthesis of certain bifunctional compounds of the disclosure.

FIG. 85 illustrates the synthesis of DNP-OH3.

FIGS. 86A-86C illustrate DNP-GN₃ meditation of the formation of a ternary complex. FIG. 86A: DNP-GN₃ mediates the formation of a ternary complex between hepatocyte cells and α-DNP antibody. FIG. 86B: DNP-GN₃-mediated ternary complex formation is inhibited by competitive binders of either ASGPR or α-DNP antibody. FIG. 86C: ternary complex formation mediated by DNP-GN₃ is inhibited by reported ASGPR-binding proteins asialofetuin and asialoorosomucoid.

FIGS. 87A-87B illustrate α-DNP antibody endocytosis is dependent on the concentrations of both α-DNP antibody and DNP-GN₃. FIG. 87A: α-DNP antibody endocytosis after six hours. FIG. 87B: α-DNP antibody endocytosis after twelve hours.

FIG. 88 illustrates that endocytosis mediated by DNP-GN₃ is decreased by competitive binders of either ASGPR or α-DNP antibody. Controls are grey, compounds expected to inhibit the proposed mode of action of DNP-GN₃ are blue, and compounds not expected to inhibit are red. Data are presented as mean f SD of 9 replicates over four experiments. Statistical differences were determined by Kruskall-Wallace test with post-hoc comparisons between each inhibitor and the no-inhibitor group (*P<0.05, **P<0.01, ***P<0.001, ****P<0.0001, “n.s”P>0.9999).

FIG. 89 illustrates that inhibitors of clathrin-dependent endocytosis decrease DNP-GN₃-mediated α-DNP antibody uptake. Data are presented as mean t SD of 9 replicates over four experiments. Statistics were performed as outlined in FIG. 88.

FIG. 90 illustrates accumulation of α-DNP antibody-derived fluorescence in cells is dependent on the presence of α-DNP antibody and DNP-GN3.

FIGS. 91A-91B illustrate that endocytosed α-DNP antibody is trafficked to lysosomes after 12 hours. FIG. 91A: endocytosed α-DNP antibody does not colocalize with the early endosome marker EEA1. FIG. 91B: endocytosed α-DNP antibody colocalizes with the late endosome and lysosome protein LAMP2 in cells.

FIGS. 92A-92B illustrate accumulation studies of α-DNP antibody-derived protein fragments. FIG. 92A: α-DNP antibody-derived protein fragments accumulate in cell lysates over time. FIG. 92B: cell supernatants do not accumulate fragments of α-DNP antibody over time.

FIGS. 93A-93C illustrate that DNP-GN3 and DNP-OH3 are not toxic to mice at all tested concentrations. FIG. 93A: mouse body weight following treatment with DNP-GN3 or DNP-OH3. Statistical differences were analyzed by T test. FIG. 93B: levels of aspartate transaminase (AST) in treated mice. Dashed lines represent the normal range. FIG. 93C: levels of alanine transaminase (ALT) in treated mice. Dashed lines represent the normal range.

FIG. 94 illustrates that serum levels of α-DNP antibody decrease more rapidly following repeated treatment with DNP-GN3. Serum antibody levels were measured using an ELISA assay. Each experimental group contained three mice. Statistical differences in experiments involving in vivo depletion of α-DNP antibody were assessed by repeated measures two-way ANOVA with Tukey's tests for post-hoc comparison of simple effects between each of the treatment groups and PBS.

FIG. 95 illustrates that significant decreases in serum levels of α-DNP antibody are observed after treatment with DNP-GN3, but not DNP-OH3. Each experimental group contained at least five mice.

FIG. 96 illustrates that a single dose of DNP-GN3 mediates a decrease in serum levels of α-DNP antibody. Each experimental group contained at least eight mice. Statistical differences were assessed by repeated measures two-way ANOVA with Tukey's tests for post-hoc comparison of simple effects between each of the treatment groups and PBS.

FIG. 97 illustrates that treatment with DNP-GN3 accelerates the depletion of polyclonal α-DNP antibody from serum. The PBS treated group contained two mice, while the DNP-GN3 group contained three mice.

FIG. 98 illustrates the association of α-DNP antibody with HepG2 cells is dependent on the concentration of DNP-AF3. Error bars represent the SD of three biological replicates.

FIG. 99 illustrates that DNP-AF3 mediated antibody association with HepG2 cells is inhibited by increasing concentrations of the α-DNP antibody binding control DNP-OH3. Error bars represent the standard deviation of three biological replicates.

FIG. 100 illustrates that DNP-AF3-mediated α-DNP antibody association with HepG2 cells is inhibited by increasing concentrations of the ASGPR binding control monomeric sugar AF. Error bars represent the standard deviation of three biological replicates.

FIG. 101 illustrates that DNP-AF3-mediated α-DNP antibody association with HepG2 cells is inhibited by increasing concentrations of the ASGPR binding protein ASOR, but not ORM. Error bars represent the standard deviation of three biological replicates.

FIG. 102 illustrates that DNP-AF3-mediated α-DNP antibody association with HepG2 cells is not inhibited by increasing concentrations of the ASGPR binding protein ASF or the protein fetuin. Data points represent a single flow cytometry experiment at each concentration. Data is represented as mean fluorescence intensity of the cell population because internal controls for 100% and 0% ternary complex formation were not included in this assay.

FIGS. 103A-103C illustrate that DNP-AF3-mediated α-DNP antibody endocytosis is dependent on the concentration of both α-DNP antibody and DNP-AF3. Each data point represents an individual biological experiment. FIG. 103A: DNP-AF3-mediated α-DNP antibody endocytosis after six hours. FIG. 103B: DNP-AF3-mediated α-DNP antibody endocytosis after twelve hours. FIG. 103C: DNP-AF3-mediated α-DNP antibody endocytosis after 24 hours.

FIG. 104 illustrates that intracellular fluorescence arising from DNP-AF3-mediated α-DNP antibody endocytosis increases over time. α-DNP antibody was present at a concentration of 100 nM. Each data point represents an individual biological experiment.

FIG. 105 illustrates that DNP-AF3-mediated antibody endocytosis by HepG2 cells is inhibited by competitive binders of both ASGPR and α-DNP antibody. Antibody was present at a concentration of 100 nM, and DNP-AF3 was present at a concentration of 40 nM. Error bars represent the standard deviation of three biological replicates. Significance was analyzed using a one-way ANOVA performing multiple comparisons to the no additive control. P values are as follows: ASOR p=0.0040 (**); ORM p=0.8879 (n.s.); ASF p=0.0032 (**); Fetuin p=0.7709 (n.s.); DNP-OH3 p=0.0073 (**), GalNAc p=0.0054 (**); AF p=0.0069 (**).

FIG. 106 illustrates that DNP-AF3 mediated antibody endocytosis by HepG2 cells is inhibited by inhibitors of clathrin-mediated endocytosis and by global endocytosis inhibitors. Antibody was present at a concentration of 100 nM, and DNP-AF3 was present at a concentration of 40 nM. Each data point represents an individual biological experiment. Black bars represent control conditions, red represent metabolic poisons, green represents inhibitors of phagocytosis and macropinocytosis, blue represent caveolin-dependent endocytosis inhibitors, and grey represents clathrin-dependent endocytosis inhibitors. Significance was analyzed using a one-way ANOVA performing multiple comparisons to the no additive control. P values are as follows: NaN₃/DOG p=0.0001 (****); CytD p=0.0710 (n.s.); EIPA p=0.9994 (n.s.); Amiloride p=0.4812 (n.s.); Nystatin p=0.9997 (n.s.); Indomethacin p=0.6682 (n.s.); Genistein p=0.8406 (n.s.); NH4Cl p=0.0001 (****); Monensin p=0.0001 (****); Primaquine p=0.0001 (****); Chloroquine p=0.0001 (****); Bafilomycin p=0.0001 (****)

FIG. 107 illustrates that endocytosed α-DNP antibody accumulates in punctae within HepG2 cells over time. Antibody was present at a concentration of 100 nM, and DNP-AF3 was present at a concentration of 40 nM. The number and darkness of punctae increases over time.

FIG. 108 illustrates that both DNP-AF3 and α-DNP antibody are necessary for the observation of fluorophore-containing punctae within HepG2 cells. In the absence of either DNP-AF3 or α-DNP antibody, punctae are not observed.

FIG. 109 illustrates that fluorescent signal arising from endocytosed α-DNP antibody does not colocalize with the early endosome protein EEA 1.

FIG. 110 illustrates that the fluorescent signal arising from endocytosed α-DNP antibody colocalizes with the late endosome and lysosome protein LAMP2.

FIG. 111 illustrates the direct fluorescence visualization of α-DNP antibody protein fragments in samples collected from cell culture supernatants. α-DNP antibody was present at a concentration of 100 nM, and DNP-AF3 was present at a concentration of 40 nM.

FIG. 112 illustrates the direct fluorescence visualization of α-DNP antibody in samples collected from cell culture lysates. Low accumulation of α-DNP antibody in cell lysates was observed in samples not treated with DNP-AF3. In contrast, DNP-AF3-treated cells demonstrated a time-dependent increase in α-DNP antibody-derived Alexa 488 signal.

FIG. 113 illustrates a ratiometric visualization of the intensity of fluorescence arising from α-DNP antibody fragments. Densitometry was performed using photoshop image analysis.

FIG. 114 illustrates that the intensity of fluorescence arising from different molecular weight proteins in cell lysates changes over time. Error bars represent the SD of three biological replicates.

FIG. 115 illustrates a ratiometric representation of the accumulation of lower molecular weight Alexa 488-modified protein fragments in cell lysates. At 12 hours, the band at 25 kDa becomes brighter than the band at 50 kDa. Error bars represent the SD of three biological replicates.

FIG. 116 illustrates the effect of different proteases inhibitors on degradation of endocytosed α-DNP antibody. A lower ratio signifies more degradation, while a higher ratio signifies less degradation. Data points represent a single biological replicate.

FIG. 117 illustrates the effect of proteases inhibitors on degradation of endocytosed α-DNP antibody in HepG2 cells. A lower ratio signifies more degradation. Error bars represent the SD of three biological replicates.

FIG. 118 illustrates the effect of selected proteases inhibitors on degradation of endocytosed α-DNP antibody in HepG2 cells. A lower ratio signifies more degradation. Error bars represent the SD of three biological replicates. Significance was analyzed using a one-way ANOVA performing multiple comparisons to the no protease inhibitor control. P values are as follows: Leupeptin p=0.0504 (n.s.); E64 p=0.0461 (*); Pepstatin p=0.9419 (n.s.); Antipain p=0.0252 (*); ALLN 100 p=0.0418 (*); ALLN 10 p=0.0267 (*).

FIGS. 119A-119B illustrate the synthesis of bifunctional molecule MIF-GN3.

FIG. 120 illustrates the synthesis of MIF inhibitor 3w.

FIG. 121 illustrates the synthesis of MIF-binding bifunctional molecule MIF-PEG2-GN3.

FIG. 122 illustrates the synthesis of MIF-binding bifunctional molecule MIF-PEG4-GN3.

FIG. 123 illustrates the synthesis of MIF-binding bifunctional molecule MIF-NVS-PEG3.

FIG. 124 illustrates the synthesis of MIF-binding bifunctional molecule MIF-AF1.

FIG. 125 illustrates synthesis of MIF-binding bifunctional molecule MIF-AF2.

FIG. 126 illustrates the synthesis of MIF-binding bifunctional molecule MIF-AF3.

FIG. 127 illustrates structures of the small molecules analyzed for inhibition of mouse MIF's enzymatic activity.

FIGS. 128A-128B illustrate MIF depletion studies from cell culture supernatant. FIG. 128A: bifunctional MIF-binding molecules mediate the depletion of huMIF from cell culture supernatant. FIG. 128B: the MIF inhibitor 3w does not mediate MIF depletion from cell culture supernatant.

FIG. 129 illustrates bifunctional MIF-binding molecules with optimized ASGPR-binding motifs deplete MIF from cell culture supernatant. Error bars represent the standard deviation of nine biological replicates. Human MIF protein was present at a concentration of 100 nM.

FIG. 130 illustrates that MIF-GN3 mediates the endocytosis of fluorescently labeled human MIF protein. Each data point represents the average of three biological replicates. Error bars represent the standard deviation.

FIG. 131 illustrates that MIF-GN3 mediates the uptake of fluorescently labeled MIF protein across a broad range of target protein concentrations. Each value represents a single biological replicate.

FIG. 132 illustrates that MIF-GN3 mediated MIF endocytosis by HepG2 cells is inhibited by inhibitors of clathrin-mediated inhibitors. Antibody was present at a concentration of 100 nM, and MIF-GN3 was present at a concentration of 200 nM. Each data point represents an individual biological experiment. Black bars represent control conditions, red represent metabolic poisons, green represents inhibitors of phagocytosis and macropinocytosis, blue represent caveolin-dependent endocytosis inhibitors, and grey represents clathrin-dependent endocytosis inhibitors. Values represent the average of three biological replicates. Error bars represent the standard deviation. Statistical differences were determined by Kruskall-Wallace test with post-hoc comparisons between each inhibitor and the no-inhibitor group (*P<0.05, **P<0.01, ***P<0.001, ****P<0.0001, “n.s” P>0.9999).

FIG. 133 illustrates that cells treated with MIF-GN3 and exogenous MIF accumulate MIF punctate in cells. MIF signal shows strong colocalization with LAMP2, but not with EEA1. MIF was present at a concentration of 100 nM, and MIF-GN3 was present at a concentration of 200 nM.

FIG. 134 illustrates that cells treated with MIF-GN3 showed more rapid clearance of human MIF from circulation in mice four hours after treatment. Each data point represents the average and SD of three mice in each arm. T test analysis of the four hour time point did not show a significant difference (p=0.1525).

FIG. 135 illustrates that cells treated with MIF-GN3 showed more rapid clearance of human MIF from circulation in mice. Each data point represents the average of two (MIF i.p. PBS arm) or three serum sample readings (all other arms).

FIG. 136 illustrates that mice treated with MIF-GN3 demonstrate rapid clearance of human MIF from circulation at early time points. Each data point represents the average of the concentration of MIF in serum collected from four or five mice. Statistical differences were assessed by repeated measures two-way ANOVA with Tukey's tests for post-hoc comparison of simple effects between each of the treatment groups and PBS.

FIG. 137 illustrates that treatment of mice with MIF-GN3 did not decrease serum levels of mouse MIF. Each point represents the average of 10 serum samples and error bars are SD. Animals received a single dose of MIF-GN3 immediately following the zero hour time point.

FIG. 138 illustrates that administration of MIF-GN3 and an α-MIF antibody slow PC3 human prostate cancer cell growth in mice. Each arm is composed of five mice (except for DNP-GN₃, which has four).

FIG. 139 illustrates that administration of MIF-GN3 and an α-MIF antibody decrease the levels of circulating human MIF protein in mice injected with PC3 prostate cancer cells. Each arm is composed of five mice (except for DNP-GN3, which has four).

FIG. 140 illustrates that administration of MIF-GN3 and an α-MIF antibody enhance the survival of mice injected with human prostate cancer PC3 cells. Each arm is composed of five mice (except for DNP-GN3, which has four).

FIG. 141 illustrates the synthesis of bifunctional molecule FcIII-BCN-GN3.

FIG. 142 illustrates that FcIII-GN3 mediates the endocytosis of human IgG across a range of concentrations. The fluorescence of a population of cells treated with human IgG but not compound is subtracted from these samples to account for cellular autofluorescence and non-small molecule mediated endocytosis. Each data point represents the average of three biological replicates. Error bars represent the standard deviation.

FIG. 143 illustrates that FcIII-BCN-GN3 mediates the endocytosis of IgG over time and across a range of concentrations. Each data point represents a single biological replicate.

FIG. 144 illustrates that intracellular human IgG-derived fluorescence is increased in the presence of FcII-GN3. IgG was present at a concentration of 100 nM; FcIII-GN3 was present at a concentration of 200 nM.

FIG. 145 illustrates that FcIII-GN3 mediates the lysosomal trafficking of human IgG. IgG was present at a concentration of 100 nM; FcIII-GN3 was present at a concentration of 200 nM. The blue channel represents Hoechst nuclear stain.

FIGS. 146A-146B illustrate fragments of a bifunctional molecule which binds TNF. FIG. 146A: the TNF binder. FIG. 146B: the synthesis of the -[CON]ₕ-[Linker]ᵢ-[CON]_{h′}-[CRBM]_{i′} fragment of a molecule of formula (II).

FIGS. 147A-147B illustrate characterization of the TNF binder of FIG. 159A. FIG. 147A: mass spectrum of the TNF binder. FIG. 147B: HPLC purification of the TNF binder.

### DETAILED DESCRIPTION OF THE DISCLOSURE

The present disclosure provides, in one aspect, bifunctional compounds that can be used to promote and/or enhance degradation of an extracellular protein (or “Protein”, which may be, in a non-limiting example, a circulating protein and/or a cell surface protein, which can be attached or embedded in the cell membrane) in a subject. In certain embodiments, treatment or management of the disease and/or disorder contemplated in the disclosure requires degradation, removal, and/or reduction in concentration of the extracellular protein in the subject. Thus, in certain embodiments, administration of a compound of the disclosure to the subject removes the extracellular protein and/or reduces the circulation concentration of the extracellular protein, thus treating, ameliorating, and/or preventing the disease and/or disorder in the subject. In some embodiments, the extracellular protein comprises TNF. In some embodiments, the extracellular protein is TNF.

In certain embodiments, the compound of the disclosure comprises a group that binds to the extracellular protein. In other embodiments, the compound of the disclosure further comprises another group (such as but not limited to a small molecule) that binds to a cellular receptor, whereby the binding leads to endocytosis of the compound (and/or the extracellular protein-compound complex). The receptor binder and the extracellular protein binder can be linked via a linker such as a polyethylene glycol (PEG), any other linker as described herein with adjustable length, or other linker as described herein and containing contains one or more connector molecule(s), which are referred to herein as CON. Once the extracellular protein-compound complex undergoes endocytosis, the extracellular protein is eventually degraded, and the compound may be degraded or recycled to the outside of the cell. In some embodiments, the extracellular protein is TNF and the compound of the disclosure is a TNF binder.

The present disclosure provides, in another aspect, bifunctional compounds that can be used to promote or enhance degradation of certain autoantibodies of interest. In certain embodiments, the autoantibody mediates a disease and/or disorder in a subject, and treatment or management of the disease and/or disorder requires degradation, removal, or reduction in concentration of the autoantibody in the subject. Thus, in certain embodiments, administration of a compound of the disclosure to the subject removes the autoantibody and/or reduces the circulation concentration of the autoantibody, thus treating, ameliorating, or preventing the disease and/or disorder in the subject.

In certain embodiments, the compound of the disclosure comprises another group (such as but not limited to a small molecule) that binds to a cellular receptor, whereby the binding leads to endocytosis of the compound (and/or the extracellular protein-compound complex). Further, in certain embodiments, the compound of the disclosure comprises an autoantibody-targeting moiety (AATM), such as but not limited to a autoantibody ligand, such as but not limited to a small molecule, peptide, and/or nucleic acid aptamer, which can bind to the autoantibody of interest. The receptor binder and the AATM can be linked via a linker such as a polyethylene glycol (PEG), any other linker as described herein with adjustable length, or other linker as described herein and containing contains one or more connector molecule(s), which are referred to herein as CON. Once the autoantibody-compound complex undergoes endocytosis, the autoantibody is eventually degraded, and the compound may be degraded or recycled to the outside of the cell.

Without wishing to be limited by any theory, the bifunctional compounds of the disclosure that can be used to promote or enhance degradation of certain autoantibodies of interest have distinctive advantages over existing methods of eliminating autoantibodies from a subject. The AATM provides specificity to the bifunctional compounds. By using ATMs, one can target specific populations of autoantibodies. As shown elsewhere herein, a compound of the disclosure comprising anti-DNP IgG as the model autoantibody successfully induced degradation of anti-DNP IgG injected in mice.

The present disclosure provides a molecular approach to achieve similar goals as to plasmapheresis in diseases caused by autoantibodies. Unlike plasmapheresis, the present technology can be easily administered by various medical professionals (not just those specialized in transfusion medicine). Since the present approach is based on small molecules derived from synthetic approaches, the present disclosure circumvents the need for expensive equipment and materials and complex manufacturing practices. Compared to plasmapheresis and IVIG, the present approach is more cost-effective, safer, and accessible to patients.

Further, the present disclosure affords routes of administration that are less invasive and safer compared to extracorporeal procedures, which may introduce additional complications. The presently described compounds are modular and versatile. The targeting motifs on either ends of the linker (CRBM and AATM) can be modified to bind to various autoantibodies of interest with great specificity. Further, the defined composition of the present compounds enables simpler and consistent manufacturing practices—reducing batch to batch variability. The fact that the AATM predictably binds to the autoantibody allows for prediction of treatment outcome, drug-drug interactions, and possible side effects.

In certain embodiments, the receptor is a hepatocyte asialoglycoprotein receptor (ASGPR). In that case, the binding moiety is referred to herein as ASGPR binding moiety, or ASGPRBM. The disclosure is not limited to the receptor, but rather contemplates the use of other receptor described herein or any other endocytic receptor known in the art.

Further, the disclosure is not limited to degradation performed in hepatocytes. Rather, the disclosure contemplates that non-hepatic cells in the body display certain degradation receptors, and those receptors are contemplated within the present disclosure.

In one aspect, the compounds of the disclosure bind to an extracellular protein and/or an autoantibody and cause it to be removed from circulation in the body (and from the body) through the liver. In some embodiments, the extracellular protein is extracellular TNF. Thus, the compounds of the disclosure harness the body's own machinery for degrading proteins and/or autoantibodies. Without wishing to be limited by any theory, the compounds of the disclosure bind to certain receptors located in certain cells, such as but not limited to hepatocytes, such as but not limited to ASGPR. Such binding triggers degradation of protein targets via endolysosomal proteolysis. As a consequence of this mechanism, there is a lowering in the circulating levels of the extracellular protein target and/or extracellular autoantibody. In some embodiments, the extracellular protein target is TNF. As a result, the corresponding disease symptoms are attenuated and/or eliminated from the subject administered the present compounds.

The ASPGR has the function of clearing desialylated glycoproteins with exposed non-reducing D-galactose (Gal) or N-acetylgalactosamine (GalNac) as end groups. ASGPR is expressed at a level of about 500,000 per hepatocyte, and has minimal existence elsewhere in the body. Internalization of the target glycoproteins by the ASGPR has a half-life of about 3 min. The disclosed bifunctional compounds selectively bind to the extracellular protein through the compound's extracellular protein binder moiety, thus forming a protein complex. When this protein complex reaches the liver, the asialoglycoprotein receptor binding moiety (ASGPRBM) of the molecule engages the end-lysosomal pathway of hepatocytes through the ASGPR. Endosomal bound ASPGR releases the extracellular protein ligand at pH 5.4, and the ligand is eliminated from circulation by the hepatocytes. However, the ASPGR remains available for recycling; it is spared from lysosomal degradation and buds into recycling endosomes. Indeed, it can be recycled up to about 200 times with a recycling rate of about 15-20 minutes, depending on the cell line. ASPGR has a very promiscuous ligand size requirement, most likely reaching diameters of about 70 nm. For comparison, the IgM pentamer is approximately 20 nm in diameter, and thus meets the ASPGR's ligand size requirement.

The disclosures of the International Patent Applications No. PCT/US2019/026260, filed Apr. 8, 2019 (and published as WO 2019/199634 on Oct. 17, 2019), and No. PCT/US2019/026239, filed Apr. 8, 2019 (and published as WO 2019/199621 on Oct. 17, 2019), are incorporated herein in their entireties by reference.

In accordance with the present disclosure, conventional chemical synthetic and pharmaceutical formulation methods, as well as pharmacology, molecular biology, microbiology, and recombinant DNA techniques within the skill of the art may be employed. Such techniques are well-known and are otherwise explained fully in the literature.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics that are normal or expected for one cell or tissue type, might be abnormal for a different cell or tissue type.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH), —CH═C(CH₃)₂, -C(CH₃)═CH₂, -C(CH₃)═CH(CH₃), -C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C═C(CH₃), —C≡C(CH₂CH₃), —CH₂C═CH, —CH₂C═C(CH₃), and —CH₂C═C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R-NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino acid sequence variant” refers to polypeptides having amino acid sequences that differ to some extent from a native sequence polypeptide. Ordinarily, amino acid sequence variants possess at least about 70% homology, at least about 80% homology, at least about 90% homology, or at least about 95% homology to the native polypeptide. The amino acid sequence variants possess substitutions, deletions, and/or insertions at certain positions within the amino acid sequence of the native amino acid sequence.

The term “amino group” as used herein refers to a substituent of the form —NH₂, —NHR, —NR₂, —NR₃⁺, wherein each R is independently selected, and protonated forms of each, except for -NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aminoalkyl” as used herein refers to amine connected to an alkyl group, as defined herein. The amine group can appear at any suitable position in the alkyl chain, such as at the terminus of the alkyl chain or anywhere within the alkyl chain.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “antibody,” as used herein, refers to an immunoglobulin molecule that specifically binds with an antigen. Antibodies can be intact immunoglobulins derived from natural sources or from recombinant sources, and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, single-domain antibodies such as sdAb (either VL or VH), such as camelid antibodies (Riechmann, 1999, J. Immunol. Meth. 231:25-38), camelid VHH domains, composed of either a VL or a VH domain that exhibit sufficient affinity for the target, and multispecific antibodies formed from antibody fragments such as a bivalent fragment comprising two Fab fragments linked by a disulfide bridge at the hinge region, and an isolated complementarity-determining region (CDR) or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger & Hudson, 2005, Nature Biotech. 23:1126-1136). Antigen binding fragments can also be grafted into scaffolds based on polypeptides such as a fibronectin type III (Fn3) (U.S. Pat. No. 6,703,199, which describes fibronectin polypeptide minibodies). The antibody fragment also includes a human antibody or a humanized antibody or a portion of a human antibody or a humanized antibody.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

As used herein, the term “asialoglycoprotein receptor binding moiety” or “ASGPRBM” refers to a group that is capable of binding to at least one hepatocyte asialoglycoprotein receptor on the surface of a cell, such as but not limited to hepatocytes. Once the ASGPRBM, and any additional moiety to which it is attached, binds to the receptor on the surface of hepatocyte, the molecule comprising the ASGPRBM is taken into the hepatocyte via a phagocytosis mechanism wherein the molecule is at least partially degraded through lysosomal degradation.

As used herein, the term “C₆₋₁₀-C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀-C₆₋₁₀ biaryl include biphenyl and binaphthyl.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Eₘₐₓ) achieved within an assay.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

As used herein, the term “GN3” refers to the group

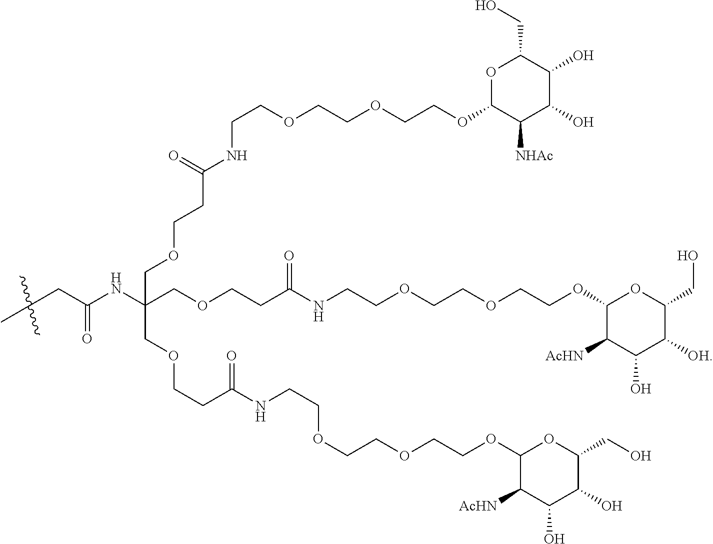 [SMILES: *CC(=O)NC(COCCC(=O)NCCOCCOCCOC1O[C@H](CO)[C@H](O)[C@H](O)[C@H]1NC(C)=O)(COCCC(=O)NCCOCCOCCO[C@@H]1O[C@H](CO)[C@H](O)[C@H](O)[C@H]1NC(C)=O)COCCC(=O)NCCOCCOCCO[C@@H]1O[C@H](CO)[C@H](O)[C@H](O)[C@H]1NC(C)=O]

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include:

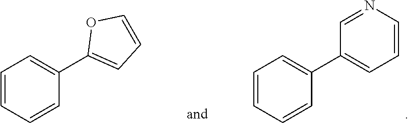 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X, X², and X³ are all the same, wherein X, X², and X³ are all different, wherein X¹ and X² are the same but X³ is different, and other analogous permutations.

The term “immunoglobulin” or “Ig” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

By the term “modulating,” as used herein, is meant mediating a detectable increase or decrease in the activity and/or level of a mRNA, polypeptide, or a response in a subject compared with the activity and/or level of a mRNA, polypeptide or a response in the subject in the absence of a treatment or compound, and/or compared with the activity and/or level of a mRNA, polypeptide, or a response in an otherwise identical but untreated subject. The term encompasses activating, inhibiting and/or otherwise affecting a native signal or response thereby mediating a beneficial therapeutic response in a subject, preferably, a human.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, the term “potency” refers to the dose needed to produce half the maximal response (ED₅₀).

As used herein, the term “Protein” refers to an extracellular protein of interest.

As used herein, the term “REAG” refers to any reagent comprising —CON, -Linker, -CON-Linker, -Linker-CON, -CON-Linker-CON, -CRBM, -CON-CRBM, -Linker-CRBM, -CON-Linker-CRBM, -Linker-CON-CRBM, and/or -CON-Linker-CON-CRBM. In certain embodiments, the REAG reacts with a TNF binder group so as to incorporate the TNF binder in the compound of the disclosure, or a fragment thereof, derivative thereof, or intermediate thereto.

The term “room temperature” as used herein refers to a temperature of about 15° C. to 28° C.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “solvent” as used herein refers to a liquid that can dissolve a solid, liquid, or gas. Non-limiting examples of solvents are silicones, organic compounds, water, alcohols, ionic liquids, and supercritical fluids.

The term “standard temperature and pressure” as used herein refers to 20° C. and 101 kPa.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “autoimmune disease” refers to a disease or illness that occurs when the body tissues are attacked by its own immune system. Examples of autoimmune diseases include, for example, systemic lupus erythematosus, Sjogren syndrome, Hashimoto thyroiditis, rheumatoid arthritis, juvenile (type 1) diabetes, polymyositis, scleroderma, Addison's disease, vitiligo, pernicious anemia, glomerulonephritis, and pulmonary fibrosis, among numerous others.

A more complete list of autoimmune diseases which may be treated by compounds and pharmaceutical compositions according to the present disclosure includes Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (APlS), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, esosiniphilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymyositis, acute disseminated encephalomyelitis (ADEM), acute motor axonic neuropathy, anti-NMDA receptor encephalitis, Balo concentric sclerosis, Bickerstaff's encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency and pyoderma gangrenosum, among others.

The term “cancer” or “neoplasia” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present disclosure may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present disclosure.

Representative common cancers to be treated with compounds according to the present disclosure include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present disclosure. Because of the activity of the present compounds, the present disclosure has general applicability treating virtually any cancer in any tissue, thus the compounds, compositions and methods of the present disclosure are generally applicable to the treatment of cancer and in reducing the likelihood of development of cancer and/or the metastasis of an existing cancer.

In certain particular aspects of the present disclosure, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present disclosure is generally applicable and may be used to treat any cancer in any tissue, regardless of etiology.

The term “anticancer agent” or “additional anticancer agent” refers to a compound other than the chimeric compounds according to the present disclosure which may be used in combination with a compound according to the present disclosure for the treatment of cancer. Exemplary anticancer agents which may be co-administered in combination with one or more chimeric compounds according to the present disclosure include, for example, antimetabolites, inhibitors of topoisomerase I and II, alkylating agents and microtubule inhibitors (e.g., taxol), among others. Exemplary anticancer compounds for use in the present disclosure may include everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (MEK) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab (Arzerra), zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid,N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolylj-quinolone, vatalanib, AG-013736, AVE-0005, the acetate salt of [D-Ser(But) 6,Azgly 10](pyro-Glu-His-Trp-Ser-Tyr-D-Ser(But)-Leu-Arg-Pro-Azgly-NH₂ acetate [C₅₉H₈₄N₁₈O₁₄—(C₂H₄O₂)_{X} wherein x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifamib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, vemurafenib among others, including immunotherapy agents such as IDO inhibitors (an inhibitor of indoleamine 2,3-dioxygenase (IDO) pathway) such as Indoximod (NLG-8187), Navoximod (GDC-0919) and NLG802, PDL 1 inhibitors (an inhibitor of programmed death-ligand 1) including, for example, nivolumab, durvalumab and atezolizumab, PD1 inhibitors such as pembrolizumab (Merck) and CTLA-4 inhibitors (an inhibitor of cytotoxic T-lymphocyte associated protein 4/cluster of differentiation 152), including ipilimumab and tremelimumab, among others.

In addition to anticancer agents, a number of other agents may be co-administered with chimeric compounds according to the present disclosure in the treatment of cancer. These include active agents, minerals, vitamins and nutritional supplements which have shown some efficacy in inhibiting cancer tissue or its growth or are otherwise useful in the treatment of cancer. For example, one or more of dietary selenium, vitamin E, lycopene, soy foods, curcumin (turmeric), vitamin D, green tea, omega-3 fatty acids and phytoestrogens, including beta-sitosterol, may be utilized in combination with the present compounds to treat cancer.

The term “inflammatory disease” is used to describe a disease or illness with acute, but more often chronic inflammation as a principal manifestation of the disease or illness. Inflammatory diseases include diseases of neurodegeneration (including, for example, Alzheimer's disease, Parkinson's disease, Huntington's disease; other ataxias), diseases of compromised immune response causing inflammation (e.g., dysregulation of T cell maturation, B cell and T cell homeostasis, counters damaging inflammation), chronic inflammatory diseases including, for example, inflammatory bowel disease, including Crohn's disease, rheumatoid arthritis, lupus, multiple sclerosis, chronic obstructive pulmonary disease/COPD, pulmonary fibrosis, cystic fibrosis, Sjogren's disease; hyperglycemic disorders, diabetes (I and II), affecting lipid metabolism islet function and/or structure, pancreatic β-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), elevated triglycerides and metabolic syndrome, liver disease, renal disease (apoptosis in plaques, glomerular disease), cardiovascular disease (especially including infarction, ischemia, stroke, pressure overload and complications during reperfusion), muscle degeneration and atrophy, low grade inflammation, gout, silicosis, atherosclerosis and associated conditions such as cardiac and neurological (both central and peripheral) manifestations including stroke, age-associated dementia and sporadic form of Alzheimer's disease, and psychiatric conditions including depression), stroke and spinal cord injury, arteriosclerosis, among others. In these diseases, elevated MIF is very often observed, making these disease states and/or conditions response to therapy using compounds and/or pharmaceutical compositions according to the present disclosure. It is noted that there is some overlap between certain autoimmune diseases and inflammatory diseases as described herein.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In one aspect, the disclosure provides a compound comprising formula (I), or a salt, geometric isomer, stereoisomer, or solvate thereof.

[Protein binder]ₖ-[CON]ₕ-[Linker]ᵢ-[CON]_{h′}-[CRBM]_{j′} (I).

In certain embodiments, the compound comprises formula (Ia), or a salt, geometric isomer, stereoisomer, or solvate thereof:

[Protein binder]-[CON]₀₋₁-[Linker]-[CON]₀₋₁-[CRBM] (Ia).

In (I) and/or (Ia), the Protein binder is a molecule, such as but not limited to a small molecule and/or a peptide, that binds to an extracellular protein of interest (“Protein”). In certain embodiments, treatment or management of the disease and/or disorder requires degradation, removal, and/or reduction in concentration of the extracellular protein in the subject. In certain embodiments, the extracellular protein binder within (I) and/or (Ia) is capable of binding to the circulating extracellular protein in the plasma of the subject with identical affinity or substantially similar affinity as compared to the extracellular protein binder itself.

In (I) and/or (Ia), the CRBM is a cellular receptor binding moiety that binds to at least one receptor on the surface of hepatocytes or other degrading cells in the subject, whereby binding of (I) or (Ia) leads to endocytosis and degradation of (I) and/or (Ia) and/or extracellular protein. In certain embodiments, the CRBM is ASGPRBM, which is a cellular receptor binding moiety that binds to at least one asialoglycoprotein receptor on the surface of hepatocytes or other degrading cells in the subject.

In (I) and/or (Ia), each CON is independently a bond or a group that covalently links a Protein binder to a CRBM, a Protein binder to a Linker, and/or a Linker to a CRBM.

In (I) and/or (Ia), the Linker is a group having a valence ranging from 1 to 15. In certain embodiments, the valence of the Linker is 1 to 10. In certain embodiments, the valence of the Linker is 1 to 5. In certain embodiments, the valence of the Linker is 1, 2, or 3. In certain embodiments, the Linker covalently links one or more CRBM and/or Protein binder groups, optionally through a CON, wherein the Linker optionally itself contains one or more CON groups.

In certain embodiments, k′ is an integer ranging from 1 to 15. In certain embodiments, k′ is an integer ranging from 1 to 10. In certain embodiments, k′ is an integer ranging from 1 to 5. In certain embodiments, k′ is an integer ranging from 1 to 3. In certain embodiments, k′ is 1, 2 or 3.

In certain embodiments, j is an integer ranging from 1 to 15. In certain embodiments, j is an integer ranging from 1 to 10. In certain embodiments, j is an integer ranging from 1 to 5. In certain embodiments, j is an integer ranging from 1 to 3. In certain embodiments, j is 1, 2 or 3.

In certain embodiments, h is an integer ranging from 0 to 15. In certain embodiments, h is an integer ranging from 1 to 15. In certain embodiments, h is an integer ranging from 1 to 10. In certain embodiments, h is an integer ranging from 1 to 5. In certain embodiments, h is an integer ranging from 1 to 3. In certain embodiments, h is 1, 2, or 3.

In certain embodiments, h′ is an integer ranging from 0 to 15. In certain embodiments, h′ is an integer ranging from 1 to 15. In certain embodiments, h′ is an integer ranging from 1 to 10.

In certain embodiments, h′ is an integer ranging from 1 to 5. In certain embodiments, h′ is an integer ranging from 1 to 3. In certain embodiments, h′ is 1, 2, or 3.

In certain embodiments, i is an integer ranging from 0 to 15. In certain embodiments, i is an integer ranging from 1 to 15. In certain embodiments, i is an integer ranging from 1 to 10. In certain embodiments, i is an integer ranging from 1 to 5. In certain embodiments, i is an integer ranging from 1 to 3. In certain embodiments, i is 1, 2, or 3.

In certain embodiments, at least one of h, h′, and i is at least 1.

In certain embodiments, k′, j′, h, h′, and i are each independently 1, 2, or 3.

In certain embodiments, k′ is 1, and j′ is 1, 2, or 3.

In another aspect, the disclosure provides a compound comprising formula (II), or a salt, geometric isomer, stereoisomer, or solvate thereof:

[TNF binder]_{k′}-[CON]ₕ-[Linker]ᵢ-[CON]_{h′}-[CRBM]_{j′} (II).

In certain embodiments, the compound comprises formula (IIa), or a salt, geometric isomer, stereoisomer, or solvate thereof:

[TNF binder]-[CON]₀₋₁-[Linker]-[CON]₀₋₁-[CRBM] (IIa).

In (II) and/or (IIa), the TNF binder is a molecule, such as but not limited to a small molecule and/or a peptide, that binds to TNF. In certain embodiments, treatment or management of the disease and/or disorder requires degradation, removal, and/or reduction in concentration of TNF in the subject. In certain embodiments, the TNF binder within (II) and/or (Ha) is capable of binding to the circulating TNF in the plasma of the subject with identical affinity or substantially similar affinity as compared to the TNF binder itself.

In (II) and/or (IIa), the CRBM is a cellular receptor binding moiety that binds to at least one receptor on the surface of hepatocytes or other degrading cells in the subject, whereby binding of (II) or (IIa) leads to endocytosis and degradation of (II) and/or (IIa) and/or TNF. In certain embodiments, the CRBM is ASGPRBM, which is a cellular receptor binding moiety that binds to at least one asialoglycoprotein receptor on the surface of hepatocytes or other degrading cells in the subject.

In (II) and/or (IIa), each CON is independently a bond or a group that covalently links a TNF binder to a CRBM, a TNF binder to a Linker, and/or a Linker to a CRBM.

In (II) and/or (IIa), the Linker is a group having a valence ranging from 1 to 15. In certain embodiments, the valence of the Linker is 1 to 10. In certain embodiments, the valence of the Linker is 1 to 5. In certain embodiments, the valence of the Linker is 1, 2, or 3. In certain embodiments, the Linker covalently links one or more CRBM and/or TNF binder groups, optionally through a CON, wherein the Linker optionally itself contains one or more CON groups.

In certain embodiments, k′ is an integer ranging from 1 to 15. In certain embodiments, k′ is an integer ranging from 1 to 10. In certain embodiments, k′ is an integer ranging from 1 to 5. In certain embodiments, k′ is an integer ranging from 1 to 3. In certain embodiments, k′ is 1, 2 or 3.

In certain embodiments, j is an integer ranging from 1 to 15. In certain embodiments, j is an integer ranging from 1 to 10. In certain embodiments, j is an integer ranging from 1 to 5. In certain embodiments, j is an integer ranging from 1 to 3. In certain embodiments, j is 1, 2 or 3.

In certain embodiments, h is an integer ranging from 0 to 15. In certain embodiments, h is an integer ranging from 1 to 15. In certain embodiments, h is an integer ranging from 1 to 10. In certain embodiments, h is an integer ranging from 1 to 5. In certain embodiments, h is an integer ranging from 1 to 3. In certain embodiments, h is 1, 2, or 3.

In certain embodiments, h′ is an integer ranging from 0 to 15. In certain embodiments, h′ is an integer ranging from 1 to 15. In certain embodiments, h′ is an integer ranging from 1 to 10. In certain embodiments, h′ is an integer ranging from 1 to 5. In certain embodiments, h′ is an integer ranging from 1 to 3. In certain embodiments, h′ is 1, 2, or 3.

In certain embodiments, i is an integer ranging from 0 to 15. In certain embodiments, i is an integer ranging from 1 to 15. In certain embodiments, i is an integer ranging from 1 to 10. In certain embodiments, i is an integer ranging from 1 to 5. In certain embodiments, i is an integer ranging from 1 to 3. In certain embodiments, i is 1, 2, or 3.

In certain embodiments, at least one of h, h′, and i is at least 1.

In certain embodiments, k′, j′, h, h′, and i are each independently 1, 2, or 3.

In certain embodiments, k′ is 1, and j′ is 1, 2, or 3.

In yet another aspect, the disclosure provides a compound comprising formula (III), or a salt, geometric isomer, stereoisomer, or solvate thereof:

[AATM]_{k′}-[CON]ₕ-[Linker]ᵢ-[CON]_{h′}-[CRBM]_{j′} (III).

In certain embodiments, the compound comprises formula (IIIa), or a salt, geometric isomer, stereoisomer, or solvate thereof:

[AATM]-[CON]₀₋₁-[Linker]-[CON]₀₋₁-[CRBM] (IIIa).

In (III) or (IIIa), the AATM is a ligand of an autoantibody. That ligand can be, for example, a small molecule, peptide, and/or nucleic acid aptamer. In certain embodiments, the autoantibody mediates a disease and/or disorder in a subject, and treatment or management of the disease and/or disorder requires degradation, removal, or reduction in concentration of the autoantibody in the subject. In certain embodiments, the AATM within (III) or (IIIa) is capable of binding to the autoantibody in the plasma of the subject with identical affinity or substantially similar affinity as compared to the AATM itself.

In (III) or (IIIa), the CRBM is a cellular receptor binding moiety that binds to at least one receptor on the surface of hepatocytes or other degrading cells in the subject, whereby binding leads to endocytosis and degradation of (III) and/or (IIIa) and/or autoantibody. In certain embodiments, the CRBM is ASGPRBM, which is a cellular receptor binding moiety that binds to at least one asialoglycoprotein receptor on the surface of hepatocytes or other degrading cells in the subject.

In (III) or (IIIa), each CON is independently a bond or a group that covalently links an AATM to an CRBM, an AATM to a Linker, and/or a Linker to a CRBM.

In (III) or (IIIa), the Linker is a group having a valence ranging from 1 to 15. In certain embodiments, the valence of the Linker is 1 to 10. In certain embodiments, the valence of the Linker is 1 to 5. In certain embodiments, the valence of the Linker is 1, 2, or 3. In certain embodiments, the Linker covalently links one or more CRBM and/or AATM groups, optionally through a CON, wherein the Linker optionally itself contains one or more CON groups.

In certain embodiments, k′ is an integer ranging from 1 to 15. In certain embodiments, k′ is an integer ranging from 1 to 10. In certain embodiments, k′ is an integer ranging from 1 to 5. In certain embodiments, k′ is an integer ranging from 1 to 3. In certain embodiments, k′ is 1, 2 or 3.

In certain embodiments, j is an integer ranging from 1 to 15. In certain embodiments, j is an integer ranging from 1 to 10. In certain embodiments, j is an integer ranging from 1 to 5. In certain embodiments, j is an integer ranging from 1 to 3. In certain embodiments, j is 1, 2 or 3.

In certain embodiments, h is an integer ranging from 0 to 15. In certain embodiments, h is an integer ranging from 1 to 15. In certain embodiments, h is an integer ranging from 1 to 10. In certain embodiments, h is an integer ranging from 1 to 5. In certain embodiments, h is an integer ranging from 1 to 3. In certain embodiments, h is 1, 2, or 3.

In certain embodiments, h′ is an integer ranging from 0 to 15. In certain embodiments, h′ is an integer ranging from 1 to 15. In certain embodiments, h′ is an integer ranging from 1 to 10. In certain embodiments, h′ is an integer ranging from 1 to 5. In certain embodiments, h′ is an integer ranging from 1 to 3. In certain embodiments, h′ is 1, 2, or 3.

In certain embodiments, i is an integer ranging from 0 to 15. In certain embodiments, i is an integer ranging from 1 to 15. In certain embodiments, i is an integer ranging from 1 to 10. In certain embodiments, i is an integer ranging from 1 to 5. In certain embodiments, i is an integer ranging from 1 to 3. In certain embodiments, i is 1, 2, or 3.

In certain embodiments, at least one of h, h′, and i is at least 1.

In certain embodiments, k′, j′, h, h′, and i are each independently 1, 2, or 3.

In certain embodiments, k′ is 1, and j′ is 1, 2, or 3.

### CRBM

### Folic Acid (Folate) Receptor:

In certain embodiments, the CRBM is folic acid, or any fragment or derivative thereof that is capable of binding to the folic acid (folate) receptor. Folate receptors bind folate and reduced folic acid derivatives and mediates delivery to the interior of cells of tetrahydrofolate, which is then converted from monoglutamate to polyglutamate forms (such as 5-methyltetrahydrofolate) as only monoglutamate forms can be transported across cell membranes. Human proteins from this family include folate receptor 1 (adult), folate receptor 2 (fetal), and folate receptor gamma.

In certain embodiments, the folic acid CRBM comprises methotrexate or a biologically active fragment thereof:

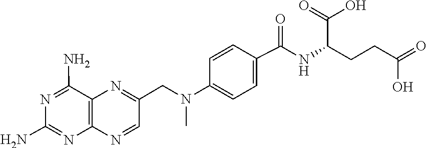 [SMILES: CN(Cc1cnc2nc(N)nc(N)c2n1)c1ccc(C(=O)N[C@@H](CCC(=O)O)C(=O)O)cc1]

In certain embodiments, the folic acid CRBM comprises premetrexed or a biologically active fragment thereof:

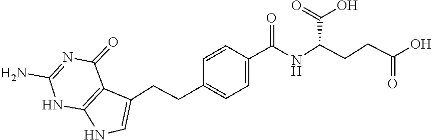 [SMILES: Nc1nc(=O)c2c(CCc3ccc(C(=O)N[C@@H](CCC(=O)O)C(=O)O)cc3)c[nH]c2[nH]1]

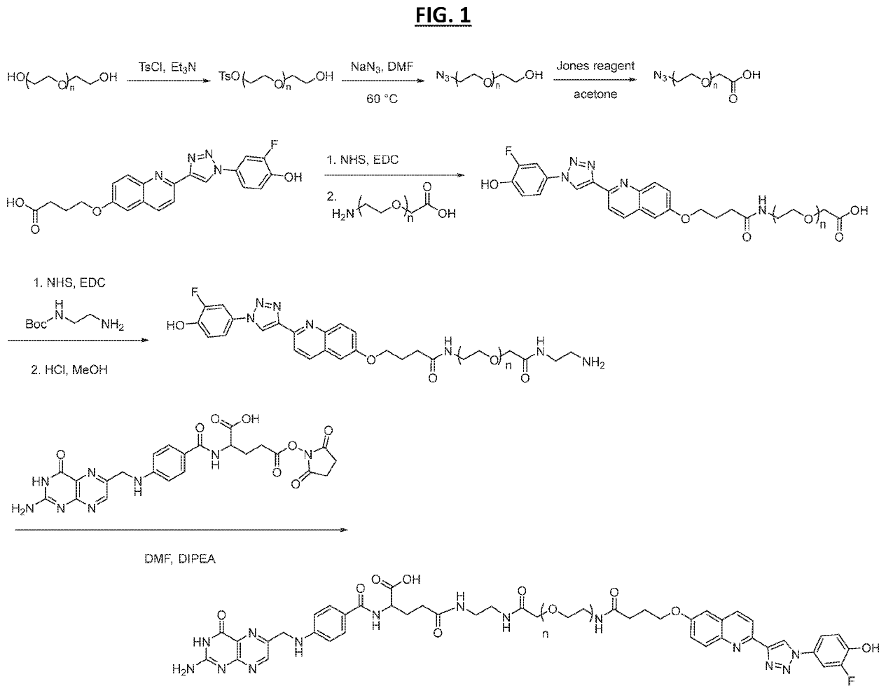

In certain embodiments, the folic acid CRBM can be incorporated into the compound of the disclosure through one of its carboxylic acid, as illustrated in FIG. 1. In other embodiments, the folic acid CRBM can be incorporated into the compound of the disclosure using N-hydroxysuccinamidyl (NHS)-activated folate, as illustrated in FIG. 1 for folic acid (similar chemistry is applicable to methotrexate and premetrexed).

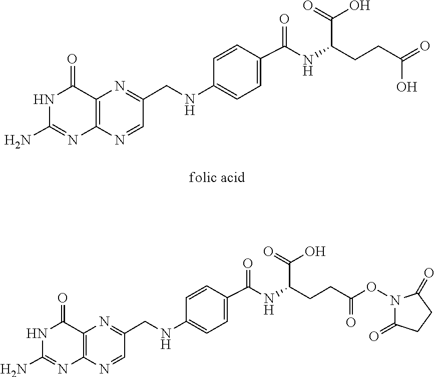 [SMILES: Nc1nc2ncc(CNc3ccc(C(=O)N[C@@H](CCC(=O)O)C(=O)O)cc3)nc2c(=O)[nH]1 ; Nc1nc2ncc(CNc3ccc(C(=O)N[C@@H](CCC(=O)ON4C(=O)CCC4=O)C(=O)O)cc3)nc2c(=O)[nH]1]

### N-Hydroxysuccinamidyl-Derivatized Folic Acid

### Mannose Receptor:

In certain embodiments, the CRBM is a group that binds to a mannose receptor. In certain embodiments, the CRBM comprises the group:

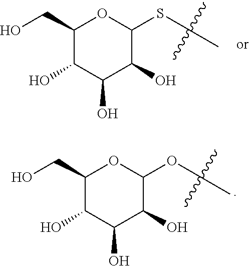 [SMILES: *SC1O[C@H](CO)[C@@H](O)[C@H](O)[C@@H]1O ; *OC1O[C@H](CO)[C@@H](O)[C@H](O)[C@@H]1O]

In certain embodiments, the mannose receptor CRBM can be attached to the compound of the disclosure (such as but not limited to the REAG) using one of the following reagents (which may be optionally protected with appropriately protecting groups):

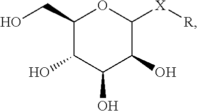

wherein X is S or O, wherein R is selected from the group consisting of:

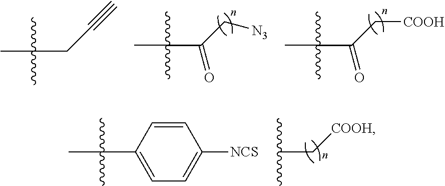

and wherein each occurrence of ‘n’ is independently 1, 2, 3, 4, 5, 6, 7, 8, 9, 10, 11, 12, 13, 14, 15, 16, 17, 18, 19, or 20.

In certain embodiments, the mannose receptor CRBM is part of a polymeric molecule. Such molecule can comprise one or more independently selected mannose receptor CRBMs as part of a polymeric chain. In certain embodiments, the CRBMs are incorporated into the polymeric molecule using CRBM reagents recited elsewhere herein.

### Mannose-6-Phosphate (M6P) Receptor:

In certain embodiments, the CRBM is a group that binds to a mannose-6-phosphate (M6P) receptor. In certain embodiments, the CRBM comprises the group:

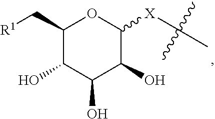

wherein X is O or S, and wherein R¹ is selected from the group consisting of:

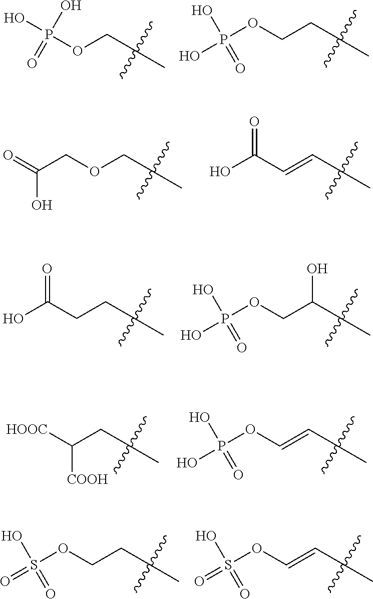

In certain embodiments, the CRBM can be attached to the compound of the disclosure (such as but not limited to the REAG) using one of the following reagents (which may be optionally protected with appropriately protecting groups):

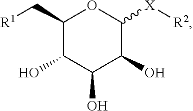

wherein X and R¹ are as defined elsewhere herein, wherein R² is selected from the group consisting of:

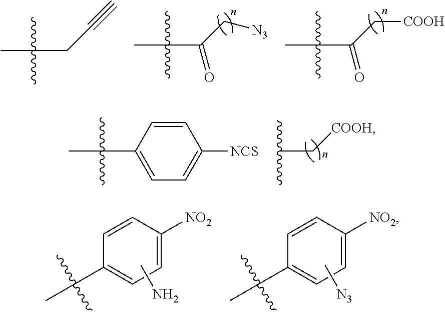

and wherein each occurrence of ‘n’ is independently 1, 2, 3, 4, 5, 6, 7, 8, 9, 10, 11, 12, 13, 14, 15, 16, 17, 18, 19, or 20.

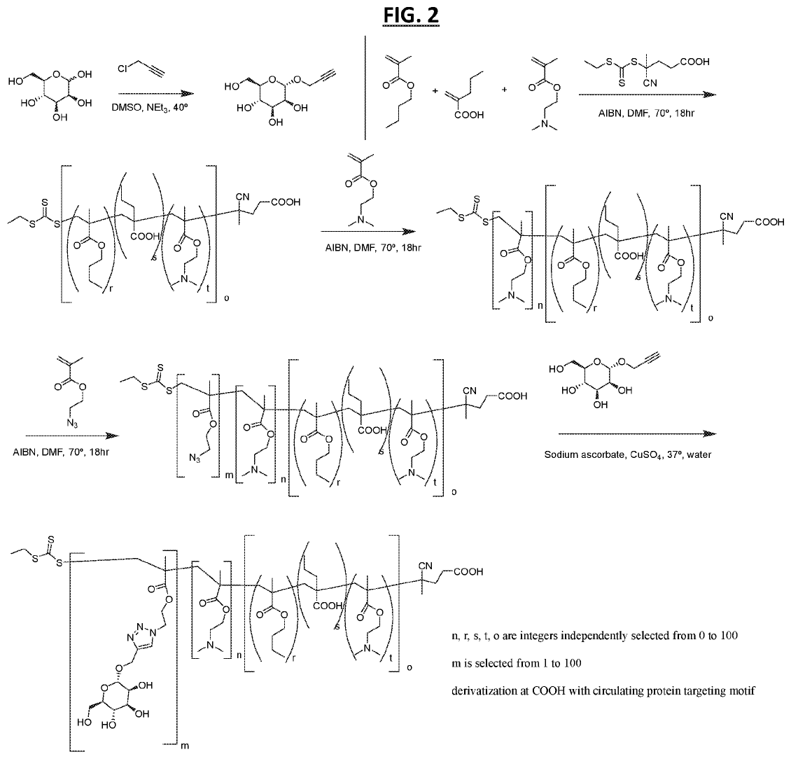

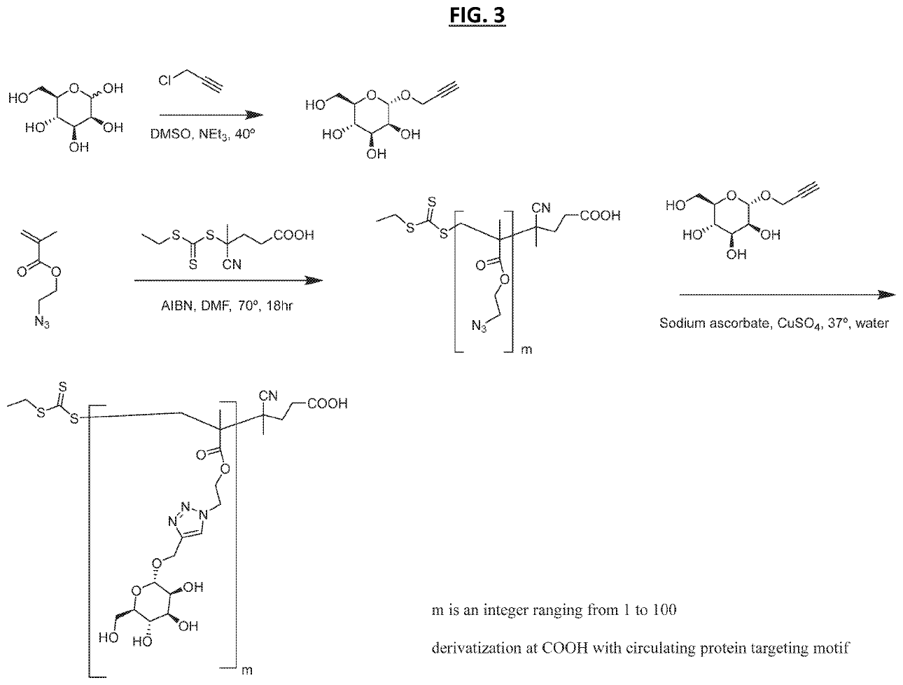

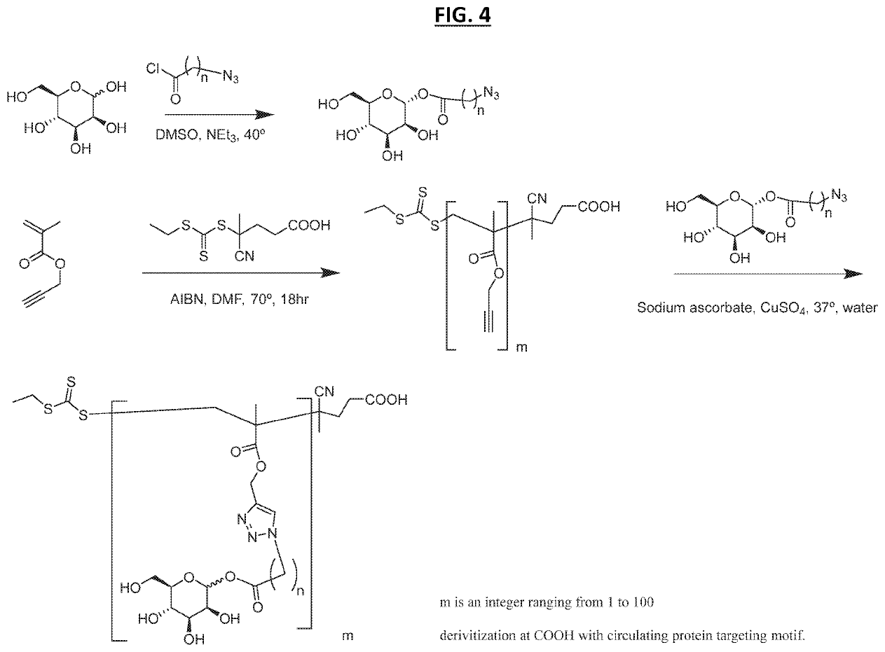

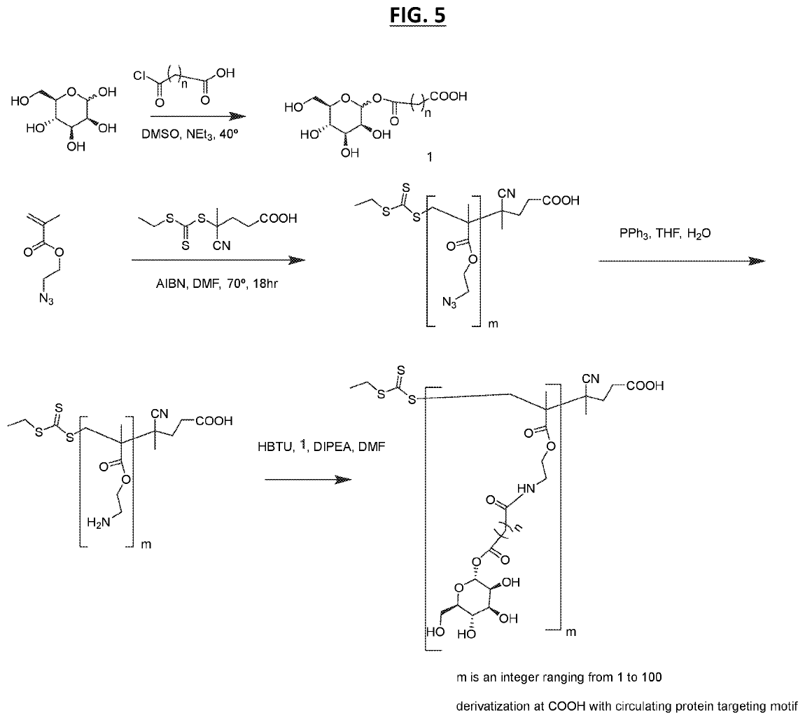

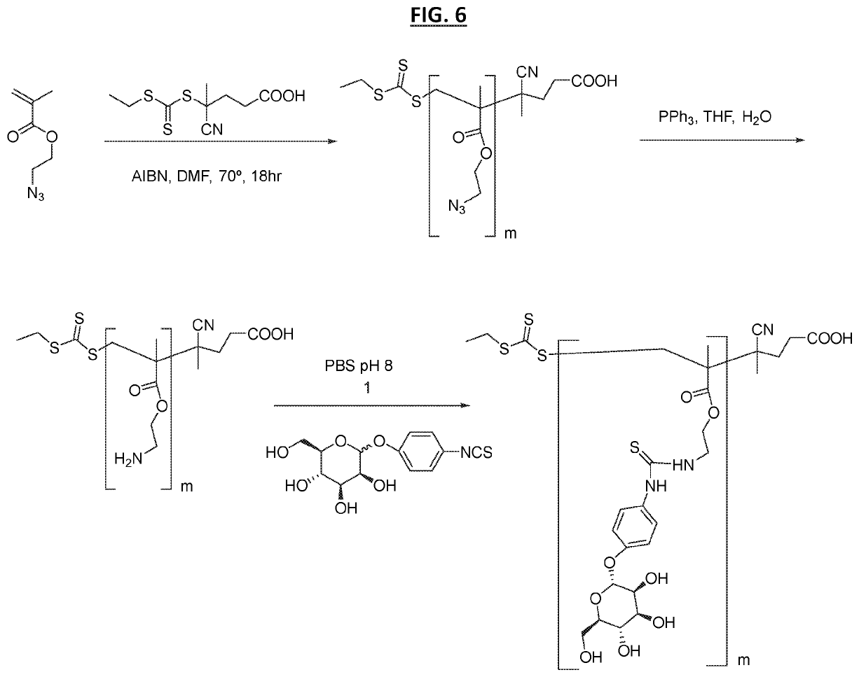

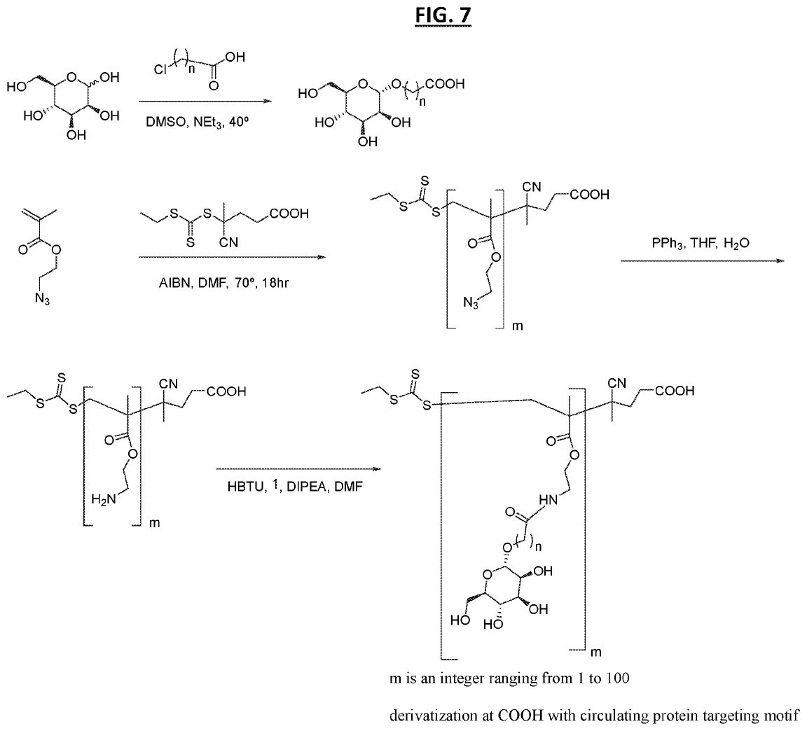

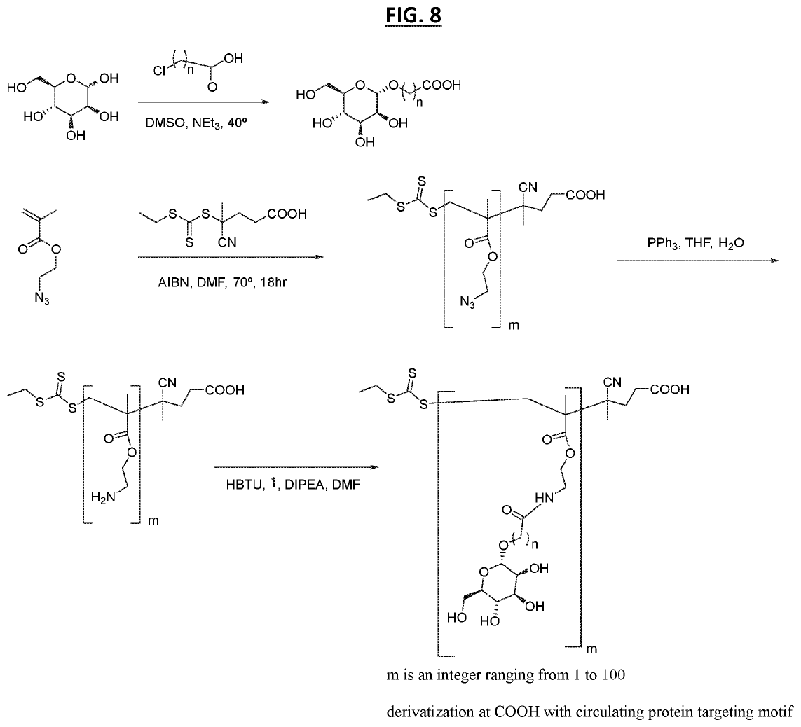

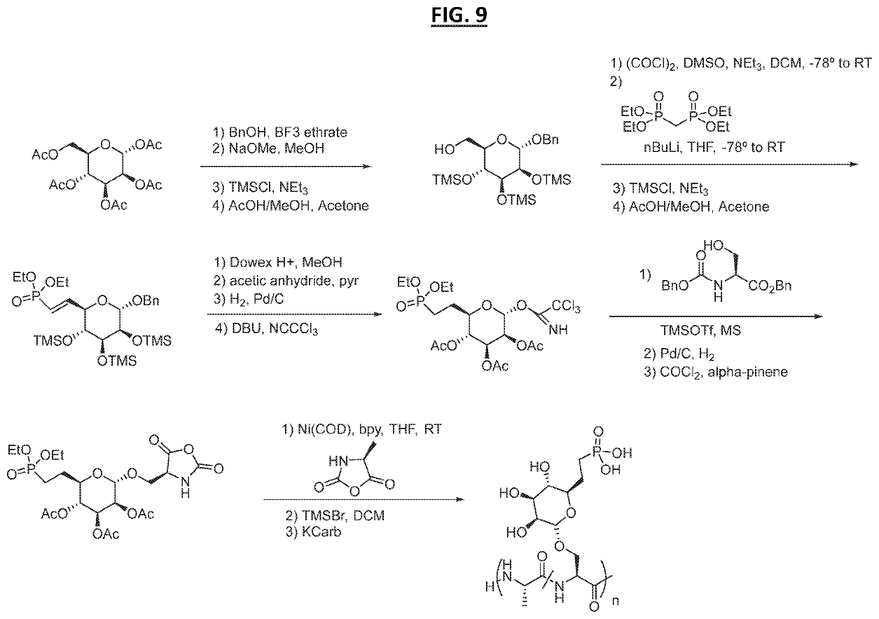

In certain embodiments, the M6P receptor CRBM is part of a polymeric molecule. Such molecule can comprise one or more independently selected M6P receptor CRBMs as part of a polymeric chain. In certain embodiments, the CRBMs are incorporated into the polymeric molecule using CRBM reagents recited elsewhere herein. FIGS. 2-9 illustrate exemplary mannose receptor binders and their preparation.

In certain embodiments, the M6P receptor CRBM is one of the following (Yamaguchi, et al., 2016, J. Am. Chem. Soc. 138(38):12472-12485):

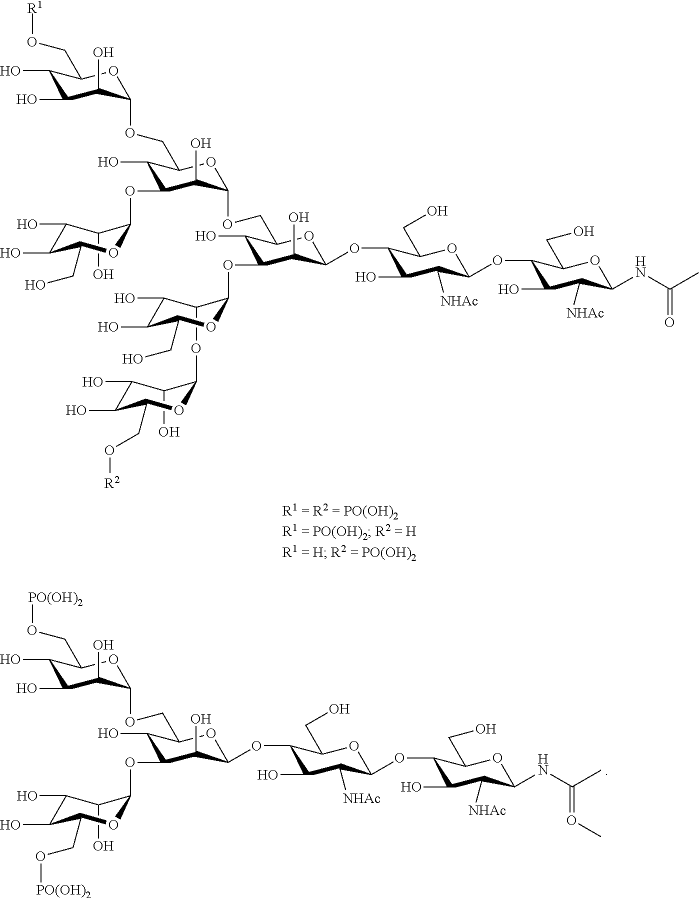

In certain embodiments, the M6P receptor CRBM is one of the following (US 2011/0110960 to Platenburg):

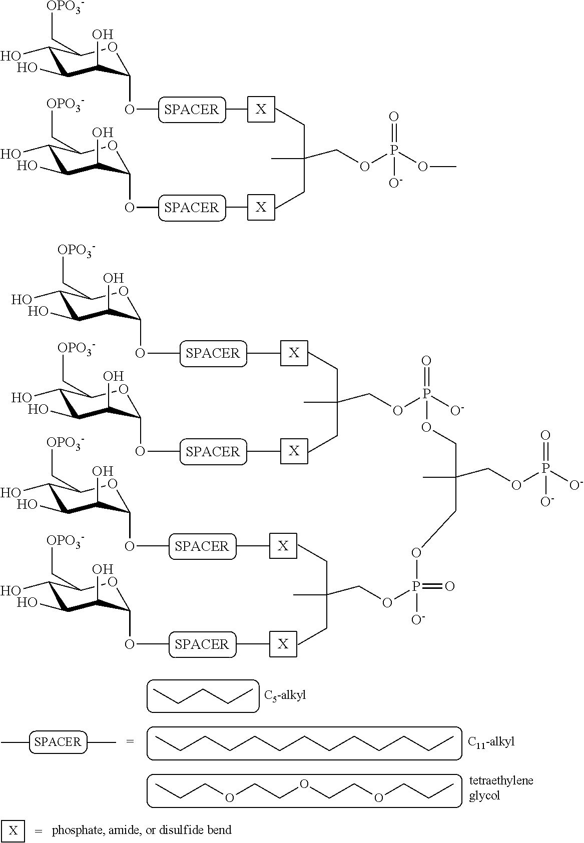

### Low Density Lipoprotein Receptor-Related Protein 1 (LRP1) Receptor:

In certain embodiments, the CRBM is a LRP1 [Low density lipoprotein receptor-related protein 1: also known as alpha-2-macroglobulin receptor (A2MR), apolipoprotein E receptor (APOER) or cluster of differentiation 91 (CD91)] binding group comprising one of the following amino acid sequences:
Ac-VKFNKPFVFLNleIEQNTK-NH₂ (SEQ ID NO:1), Toldo et al., 2017, JACC: Basic to Translational Science 2.5:561-574;
VKFNKPFVFLMTEQNTK (SEQ ID NO:2), Toldo et al., 2017, JACC: Basic to Translational Science 2.5:561-574;
TWPKHFDKHTFYSILKLGKH-OH (SEQ ID NO:3), Sakamoto, et al., 2017, Biochemistry and biophysics reports 12:135-139;
Angiopep-2: TFFYGGSRGKRNNFKTEEY-OH (SEQ ID NO:4), Sakamoto, et al., 2017, Biochemistry and biophysics reports 12:135-139;
LRKLRKRLLRDADDLLRKLRKRLLRDADDL (SEQ ID NO:5), Croy, et al., 2004, Biochemistry 43.23:7328-7335;
TEELRVRLASHLRKLRKRLL (SEQ ID NO:6), Croy, et al., 2004, Biochemistry 43.23:7328-7335;
Rap12: EAKIEKHNHYQK (SEQ ID NO:7), Ruan, et al., 2018, Journal of Controlled Release 279:306-315;
Rap22: EAKIEKHNHYQKQLEIAHEKLR (SEQ ID NO:8), Ruan, et al., 2018, Journal of Controlled Release 279:306-315; or
ANG: TFFYGGSRGKRNNFKTEEY (SEQ ID NO:9) Kim, et al., 2016, Scientific reports 6:34297.

### Low Density Lipoprotein Receptor (LDLR):

In certain embodiments, the CRBM is a LDLR (low density lipoprotein receptor) binding group comprising one of the following amino acid sequences:
VH4127: cM-Thz-RLRG-Pen (cyclized c-Pen) (SEQ ID NO:10), Molino, et al., 2017, The FASEB Journal 31.5:1807-1827;
VH434: CMPRLRGC (cyclized C-C) (SEQ ID NO: 11), Molino, et al., 2017, The FASEB Journal 31.5:1807-1827;
VH101: HLDCMPRGCFRN (cyclized C-C) (SEQ ID NO:12), David, et al., 2018, PloS one 13.2: 0191052;
VH202: CQVKSMPRC (cyclized C-C) (SEQ ID NO:13), David, et al., 2018, PloS one 13.2: 0191052;
VH203: CTTPMPRLC (cyclized C-C) (SEQ ID NO:14), David, et al., 2018, PloS one 13.2: 0191052;
VH204: CKAPQMPRC (cyclized C-C) (SEQ ID NO:15), David, et al., 2018, PloS one 13.2: 0191052;
VH205: CLNPSMPRC (cyclized C-C) (SEQ ID NO:16), David, et al., 2018, PloS one 13.2: 0191052;
VH306: CLVSSMPRC (cyclized C-C) (SEQ ID NO:17), David, et al., 2018, PloS one 13.2: 0191052;
VH307: CLQPMPRLC (cyclized C-C) (SEQ ID NO:18), David, et al., 2018, PloS one 13.2: 0191052;
VH308: CPVSSMPRC (cyclized C-C) (SEQ ID NO:19), David, et al., 2018, PloS one 13.2: 0191052;
VH309: CQSPMPRLC (cyclized C-C) (SEQ ID NO:20), David, et al., 2018, PloS one 13.2: 0191052;
VH310: CLTPMPRLC (cyclized C-C) (SEQ ID NO:21), David, et al., 2018, PloS one 13.2: 0191052;
VH411: DSGLCMPRLRGCDPR (cyclized C-C) (SEQ ID NO:22), David, et al., 2018, PloS one 13.2: 0191052;
VH549: TPSAHAMALQSLSVG (SEQ ID NO:23), David, et al., 2018, PloS one 13.2: 0191052;
AcVH411: Ac-DSGLCMPRLRGCDPR-NH2 (cyclized C-C) (SEQ ID NO:24), David, et al., 2018, PloS one 13.2: 0191052;
PrVH434: Pr-CMPRLRGC-NH₂ (cyclized C-C) (SEQ ID NO:25), David, et al., 2018, PloS one 13.2: 0191052;
VH445: Pr-cMPRLRGC-NH₂ (cyclized C-C) (SEQ ID NO:26), David, et al., 2018, PloS one 13.2: 0191052;
VH4127: Pr-cMThzRLRG-Pen-NH2 (cyclized C-Pen) (SEQ ID NO:27), David, et al., 2018, PloS one 13.2: 0191052;
AcVH434: Ac-CMPRLGC-NH₂ (cyclized C-C) (SEQ ID NO:28), Jacquot, et al., 2016, Molecular pharmaceutics 13.12:4094-4105;
AcVH445: Ac-cMPRLRGC-NH₂ (cyclized C-C) (SEQ ID NO:29), Jacquot, et al., 2016, Molecular pharmaceutics 13.12:4094-4105;
VH4106: Ac-D-Pen-M-Thz-RLRGC-NH₂ (cyclized Pen-C) (SEQ ID NO:30), Jacquot, et al., 2016, Molecular pharmaceutics 13.12:4094-4105;
VH4127: Pr-cM-Thz-RLRG-Pen-NH2 (cyclized c-Pen) (SEQ ID NO:31), Jacquot, et al., 2016, Molecular pharmaceutics 13.12:4094-4105;
VH4128: Pr-cM-Thz-RLR-Sar-Pen-NH₂ (cyclized C-Pen) (SEQ ID NO:32), Jacquot, et al., 2016, Molecular pharmaceutics 13.12:4094-4105;
VH4129: Pr-cM-Pip-RLR-Sar-C-NH₂ (cyclized C-C) (SEQ ID NO:33), Jacquot, et al., 2016, Molecular pharmaceutics 13.12:4094-4105;
VH4130: Pr-cM-Pip-RLRG-Pen-NH2 (cyclized c-Pen) (SEQ ID NO:34), Jacquot, et al., 2016, Molecular pharmaceutics 13.12:4094-4105; or
VH4131: Pr-cM-Pip-RLR-Sar-Pen-NH2 (cyclized c-Pen) (SEQ ID NO:35), Jacquot, et al., 2016, Molecular pharmaceutics 13.12:4094-4105.

### FcγRI Receptor:

In certain embodiments, the CRBM is a FcγRI binding group comprising one of the following amino acid sequences:
Cp22: TDT C LMLPLLLG C DEE (cyclized C-C) (SEQ ID NO:36), Bonetto, et al, 2009, The FASEB Journal 23 2:575-585;
Cp21: DPI C WYFPRLLG C TTL (cyclized C-C) (SEQ ID NO:37), Bonetto, et al, 2009, The FASEB Journal 23.2:575-585;
Cp23: WYP C YIYPRLLG C DGD (cyclized C-C) (SEQ ID NO:38), Bonetto, et al, 2009, The FASEB Journal 23.2:575-585;
Cp24: GNI C MLIPGLLG C SYE (cyclized C-C) (SEQ ID NO:39), Bonetto, et al, 2009, The FASEB Journal 23.2:575-585;
Cp33: VNS C LLLPNLLG C GDD (cyclized C-C) (SEQ ID NO:40), Bonetto, et al, 2009, The FASEB Journal 23.2:575-585;
Cp25: TPV C ILLPSLLG C DTQ (cyclized C-C) (SEQ ID NO:41), Bonetto, et al, 2009, The FASEB Journal 23 2:575-585;
Cp26: TVL C SLWPELLG C PPE (cyclized C-C) (SEQ ID NO:42), Bonetto, et al, 2009, The FASEB Journal 23.2:575-585;
Cp27: TFS C LMWPWLLG C ESL (cyclized C-C) (SEQ ID NO:43), Bonetto, et al, 2009, The FASEB Journal 23 2:575-585;
Cp32: FGT C YTWPWLLG C EGF (cyclized C-C) (SEQ ID NO:44), Bonetto, et al, 2009, The FASEB Journal 23.2:575-585;
Cp34: SLF C RLLLTPVG C VSQ (cyclized C-C) (SEQ ID NO:45), Bonetto, et al, 2009, The FASEB Journal 23.2:575-585;
P35: HLL V LPRGLLG C TTLA (cyclized C-C) (SEQ ID NO:46), Bonetto, et al, 2009, The FASEB Journal 23.2:575-585;
Cp28: TSL C SMFPDLLG C FNL (cyclized C-C) (SEQ ID NO:47), Bonetto, et al, 2009, The FASEB Journal 23.2:575-585;
Cp29: SHP C GRLPMLLG C AES (cyclized C-C) (SEQ ID NO:48), Bonetto, et al, 2009, The FASEB Journal 23.2:575-585;
P37: TST C SMVPGPLGAV STW (cyclized C-C) (SEQ ID NO:49), Bonetto, et al, 2009, The FASEB Journal 23.2:575-585;
Cp30: KDP C TRWAMLLG C DGE (cyclized C-C) (SEQ ID NO:50), Bonetto, et al, 2009, The FASEB Journal 23.2:575-585;
Cp31: IMT C SVYPFLLG C VDK (cyclized C-C) (SEQ ID NO:51), Bonetto, et al, 2009, The FASEB Journal 23 2.575-585;
Cp36: IHS C AHVMRLLG C WSR (cyclized C-C) (SEQ ID NO:52), Bonetto, et al, 2009, The FASEB Journal 23.2:575-585.

### Transferrin Receptor:

In certain embodiments, the CRBM is a transferrin receptor binding group comprising one of the following amino acid sequences:
Tf1: CGGGPFWWWP (SEQ ID NO:53), Sand, et al., 2016, Bioconjugate chemistry 28.2 471-480;
Tf2: CGGGHKYLRW (SEQ ID NO:54), Santi, et al., 2016, Bioconjugate chemistry 28 2:471-480;
Tf3: CGGGKRIFMV (SEQ ID NO:55), Santi, et al., 2016, Bioconjugate chemistry 28.2:471-480;
Tf2-scr: CGGGKWHYLR (SEQ ID NO:56), Santi, et al., 2016, Bioconjugate chemistry 28 2:471-480;
TfR-T12: THRPPMWSPVWP (SEQ ID NO:57), Mu, et al., 2017, Scientific reports 7.1:3487; HAIYPRH (SEQ ID NO:74), Lee, et al, 2001, European journal of biochemistry 268 7:2004-2012);
THRPPMWSPVWP (SEQ ID NO:58), Lee, et al, 2001, European journal of biochemistry 268 7:2004-2012);
THRPPMWSPVWP (SEQ ID NO:59), Wangler, et al., 2011, Molecular Imaging and Biology 13.2:332-341.

### Macrophage Scavenger Receptor:

In certain embodiments, the CRBM is a macrophage scavenger receptor binding moiety comprising one of the following amino acid sequences:
PP1: LSLERFLRCWSDAPA (SEQ ID NO:60), Segers, et al., 2012, Arteriosclerosis, thrombosis, and vascular biology 32.4:971-978;
PP1-13: LERFLRCWSDAPA (SEQ ID NO:61), Segers, et al., 2012, Arteriosclerosis, thrombosis, and vascular biology 32.4:971-978;
PP1-11: RFLRCWSDAPA (SEQ ID NO:62), Segers, et al., 2012, Arteriosclerosis, thrombosis, and vascular biology 32.4:971-978;
PP1-9: LRCWSDAPA (SEQ ID NO:63), Segers, et al., 2012, Arteriosclerosis, thrombosis, and vascular biology 32.4:971-978;
PP1-7: CWSDAPA (SEQ ID NO:64), Segers, et al., 2012, Arteriosclerosis, thrombosis, and vascular biology 32.4:971-978;
4F: DWFKAFYDKVAEKFKEAF (SEQ ID NO:65), Neyen, et al., 2009, Biochemistry 48.50:11858-11871);

As used herein, Pen is Penicillamine, Thz is thiazolidine-4-carboxylic acid, Sar is sarcosine, Pip is pipecolic acid, Nleu is norleucine, and NMeLeu is N-methylleucine.

### G-Protein Coupled Receptor:

In certain embodiments, the CRBM is a G-protein coupled receptor (GPCR) binding moiety. In certain embodiments, the binding moiety binds to the GPCR and induces receptor internalization. In certain embodiments, the receptor is CXCR7 (see, for example, Nalawansha, et al., 2019, ACS Cent. Sci. 5(6):1079-1084). In certain embodiments, the binding moiety comprises the following:

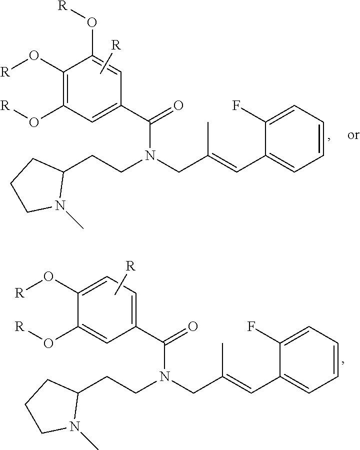

wherein each occurrence of R is independently H or C₁-C₆ alkyl. In certain embodiments, the CRBM can be attached to the compound of the disclosure (such as but not limited to the REAG) using one of the following reagents (which may be optionally protected with appropriately protecting groups):

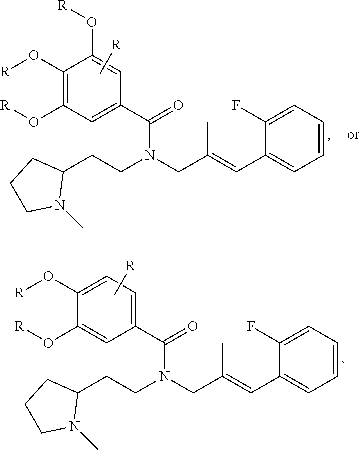

wherein at least one occurrence of R is REAG, and wherein the remaining occurrences of R are independently H or C₁-C₆ alkyl.

### Asialoglycoprotein Receptor (ASGPR):

The disclosure contemplates the use of a ASGPR binding moiety (ASGPRBM).

In certain embodiments, the ASGPRBM group is any such group recited in Huang, et al., 2017, Bioconjugate Chem. 28:283-295, which is incorporated herein in its entirety by reference.

In certain embodiments, the ASGPRBM group comprises the structure:

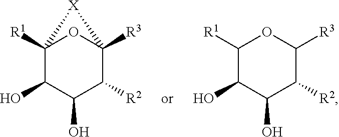

wherein X is a linker of 1-4 atoms in length and comprises O, S, N(R^{N1}), or C(R^{N1})(R^{N1}) groups, such that:
when X is a linker of 1 atom in length, X is O, S, N(R^{N1}), or C(R^{N1})(R^{N1})
when X is a linker of 2 atoms in length, no more than 1 atom of X is O, S, or N(R^{N1})
when X is a linker of 3 or 4 atoms in length, no more than 2 atoms of X are independently O, S, or N(R^{N1}).

In certain embodiments, each occurrence of R^{N1} is independently H or C₁-C₃ alkyl optionally substituted with 1-3 independently selected halogens and/or 1-2 hydroxyl groups.

In certain embodiments, the X in ASGPRBM is —O—C(R^{N1})(R^{N1})—, —C(R^{N1})(R^{N1})O—, —S—C(R^{N1})(R^{N1})—, —C(R^{N1})(R^{N1})—S—, —N(R^{N1})—C(R^{N1})(R^{N1})—, —C(R^{N1})(R^{N1})—N(R^{N1})—, or —C(R^{N1})(R^{N1})—C(R^{N1})(R^{N1})—, when X is 2 atoms in length.

In certain embodiments, the X in ASGPRBM is —O-C(R^{N1})(R^{N1})—C(R^{N1})(R^{N1})—, —C(R^{N1})(R^{N1})—O—C(R^{N1})(R^{N1})—, —O—C(R^{N1})(R^{N1})—O—, —O—C(R^{N1})(R^{N1})—S—, —O—C(R^{N1})(R^{N1})—N(R^{N1})—S—C(R^{N1})(R^{N1})—C(R^{N1})(R^{N1})—, —C(R^{N1})(R^{N1})—S—C(R^{N1})(R^{N1})—, —C(R^{N1})(R^{N1})—C(R^{N1})N)—S, —C(R^{N1})(R^{N1})—S—, —S—C(R^{N1})(R^{N1})—O—, —S—C(R^{N1})(R^{N1})—N(R^{N1})—, —N(R^{N1})—C(R^{N1})(R^{N1})—C(R^{N1})(R^{N1})—, —C(R^{N1})(R^{N1})—N(R^{N1})—C(R^{N1})(R^{N1})—, —C(R^{N1})(R^{N1})—C(R^{N1})(R^{N1})—N(R^{N1})—, —N(R^{N1})—C(R^{N1})(R^{N1})—N(R^{N1})—, or —C(R^{N1})(R^{N1})—C(R^{N1})(R^{N1})—C(R^{N1})(R^{N1}), when X is 3 atoms in length.

In certain embodiments, the X in ASGPRBM is —O—C(R^{N1})(R^{N1})—C(R^{N1})(R^{N1})—C(R^{N1})(R^{N1})—, —C(R^{N1})(R^{N1})—O—C(R^{N1})(R^{N1})—C(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})—, —C(R^{N1})(R^{N1})—S—C(R^{N1})(R^{N1})C(R^{N1})(R^{N1})—, —C(R^{N1})(R^{N1})—C(^{N1})(R^{N1})—S—C(R^{N1})(R^{N1})—, —S—C(R^{N1})(R^{N1})—S—C(R^{N1})(R^{N1})—, —N(R^{N1})—C(R^{N1})(R^{N1})—C(R^{N1})(R^{N1})—C(R^{N1})(R^{N1})—, or —C(R^{N1})(R^{N1})—N(R^{N1})—C(R^{N1})(R^{N1})—C(R^{N1})(R^{N1})—, when X is 4 atoms in length.

In certain embodiments, X is OCH₂ and R^{N1} is H.

In certain embodiments, X is CH₂O and R^{N1} is H.

In certain embodiments, the ASGPRBM comprises the structure:

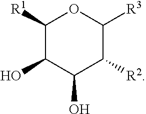

In certain embodiments, the ASGPRBM comprises the structure:

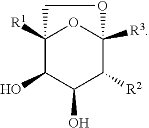

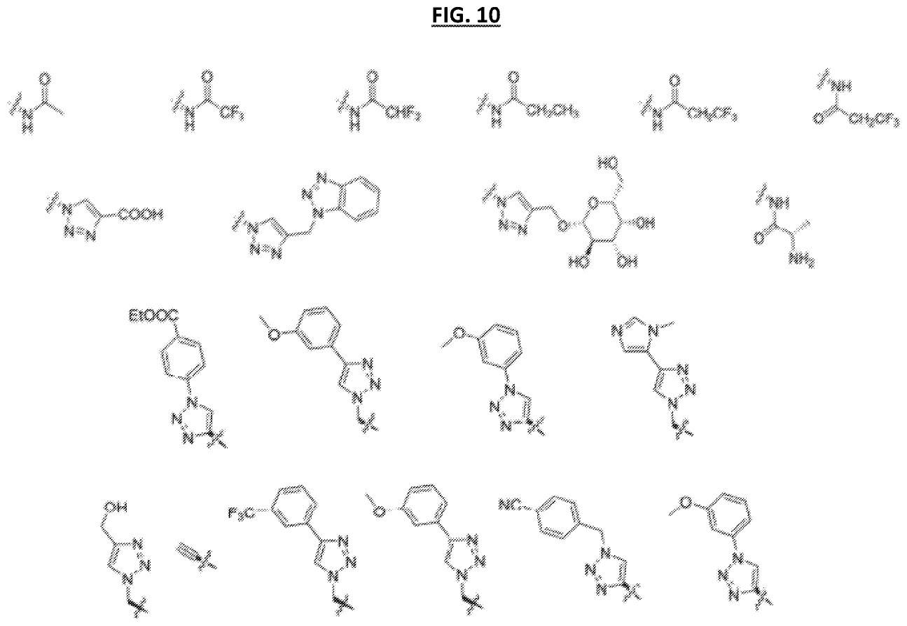

In certain embodiments, R¹ is a group depicted in FIG. 10. In certain embodiments, R; is a group depicted in FIG. 10. In certain embodiments, R¹ and R³ are each independently a group depicted in FIG. 10.

In certain embodiments, R¹ and R³ are each independently H, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}(vinyl), —O(CH₂)_{K}(vinyl), —(CH₂)_{K}(alkynyl), —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens.

In certain embodiments, R¹ and R³ are each independently Ph(CH₂)_{K}—, which is optionally substituted with: 1-3 independently selected halogens; C₁-C₄ alkyl optionally substituted with 1-3 independently selected halogens and/or 1-2 hydroxyl groups; or C₁-C₄ alkoxy optionally substituted with 1-3 independently selected halogens and/or 1-2 hydroxyl groups.

In certain embodiments, R¹ and R³ are each independently a group of structure

—O—(CH₂)_{K′}—CH(OH)—(CH₂)K′-R⁷,

wherein R⁷ is: C₁-C₄ alkoxy optionally substituted with 1-3 independently selected halogens and/or 1-2 hydroxy groups; —NR^{N3}R^{N4}; or —(CH₂)_{K}—O—(CH₂)_{K}—CH₂—CH═CH₂.

In certain embodiments, K is 0. In certain embodiments, K is 1. In certain embodiments, K is 2. In certain embodiments, K is 3. In certain embodiments, K is 4.

In certain embodiments, K′ is 1. In certain embodiments, K′ is 2. In certain embodiments, K′ is 3. In certain embodiments, K′ is 4.

In certain embodiments, each occurrence of R^{N3} is independently H or C₁-C₃ alkyl. In certain embodiments, each occurrence of R^{N3} is independently H or C₁-C₃ alkyl optionally substituted with 1-3 independently selected halogens and/or 1-2 hydroxyl groups;

In certain embodiments, each occurrence of R^{N4} is independently H, C₁-C₃ alkyl, or Ph-(CH₂)_{K}—. In certain embodiments, each occurrence of R^{N4} is independently H, C₁-C₃ alkyl optionally substituted with 1-3 independently selected halogens and/or 1-2 hydroxyl groups, or Ph-(CH₂)_{K}—.

In certain embodiments, R¹ and R³ are each independently selected from the group consisting of:
—(CH₂)_{K}OH,

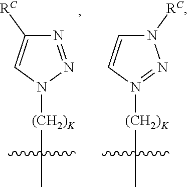

L¹-≡-, L¹-(CH₂)_{K}—, and CYC—(CH₂)_{K}—, wherein CYC is selected from the group consisting of:

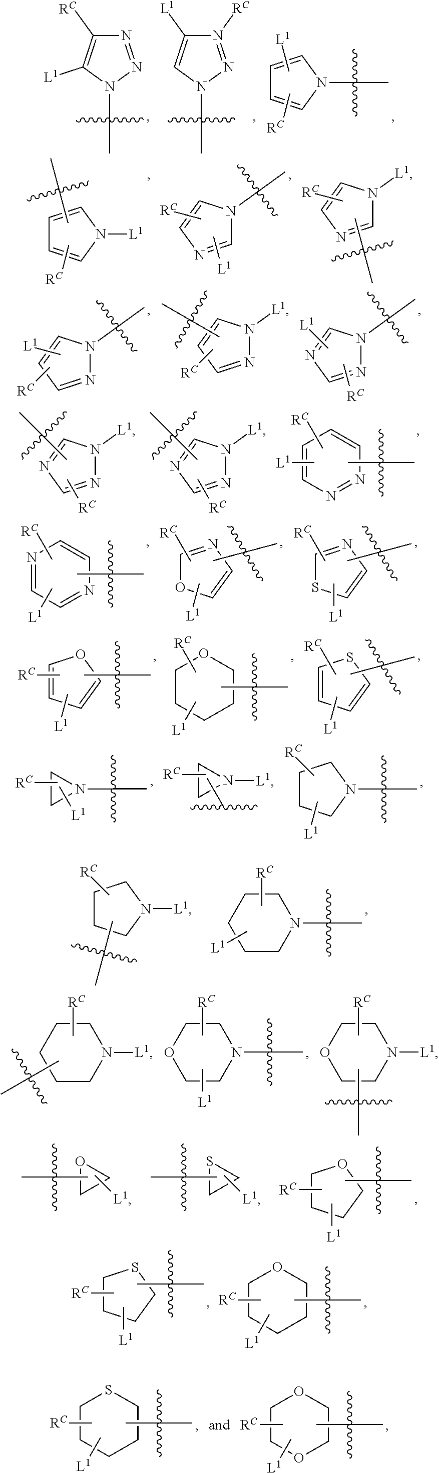

wherein the bond marked with [symbol] indicating the site on CYC whereto-(CH₂)_{K} is connected.

In certain embodiments, L¹ is a bond, -Linker, —CON-Linker, or —CON-Linker-CON. In certain embodiments, L¹ is a bond. In certain embodiments, L¹ is-Linker. In certain embodiments, L¹ is-CON-Linker. In certain embodiments, L¹ is-CON-Linker-CON.

In certain embodiments, R^{C} is absent, H, C₁-C₄ alkyl optionally substituted with 1-3 optionally substituted halogens and/or 1-2 hydroxyl groups, or a group of structure:

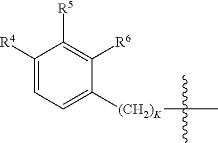

wherein R⁴, R⁵, and R⁶ are each independently H, F, Cl, Br, I, CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens, C₁-C₃-alkoxy optionally substituted with 1-3 independently selected halogens, —(CH₂)_{K}COOH, —(CH₂) KC(═O)O—(C₁-C₄ alkyl) optionally substituted with 1-3 independently selected halogens, O—C(═O)—(C₁-C₄ alkyl) optionally substituted with 1-3 independently selected halogens, or —C(═O)—(C₁-C₄ alkyl) optionally substituted with 1-3 independently selected halogens.

In certain embodiments, each occurrence of R^{N2} is independently H or C₁-C₃ alkyl optionally substituted with 1-3 independently selected halogens and/or 1-2 hydroxyl groups.

In certain embodiments, R^{C} is

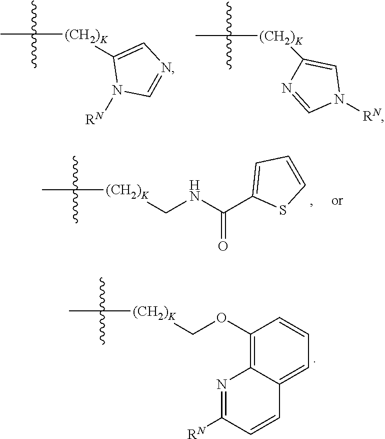

In certain embodiments, R¹ and R³ are each independently (C₃-C₈ saturated carbocyclic)-(CH₂)_{K}—, wherein the carbocyclic is further substituted with -L¹ and -R^{C}.

In certain embodiments, each occurrence of R^{N} is independently H or C₁-C₃ alkyl optionally substituted with 1-3 independently selected halogens and/or 1-2 hydroxyl groups.

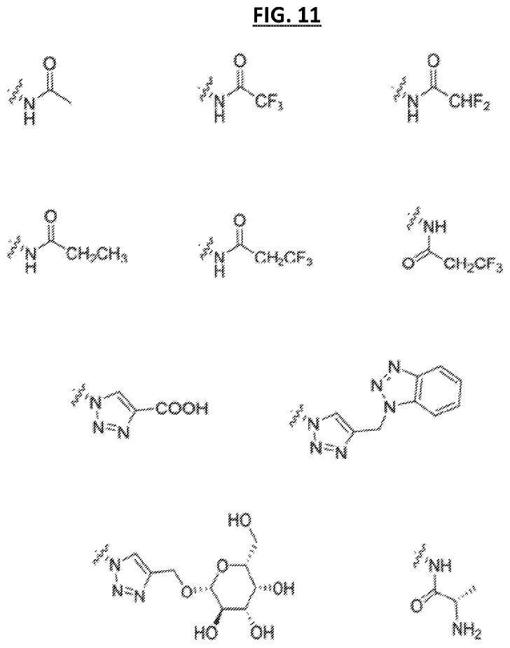

In certain embodiments, R² is a group depicted in FIG. 11.

In certain embodiments, R² is —(CH₂)_{K}—N(R^{N1})—C(═O)R^{AM}.

In certain embodiments, R^{AM} is H, C₁-C₄ alkyl optionally substituted with 1-3 independently selected halogens and/or 1-2 hydroxyl groups, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, —C(═O)(C₁-C₄ alkyl) optionally substituted with 1-3 independently selected halogens, or —(CH₂)_{K}—NR^{N3}R^{N4}

In certain embodiments, R² is

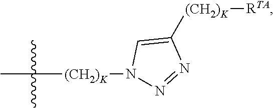

wherein:
R^{TA} is H, CN, NR^{N1}R^{N2}, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens,
or
R^{TA} is C₃-C₁₀ aryl or a 3- to 10-membered heteroaryl group containing 1-5 non-carbon ring atoms, each of the aryl or heteroaryl groups being optionally substituted with 1-3 groups independently selected from CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens and/or 1-2 hydroxyl groups, -(C₁-C₃-alkoxy) optionally substituted from 1-3 independently selected halogens, —(CH₂)_{K}COOH, —(CH₂)_{K}C(═O)O—(C₁-C₄ alkyl) optionally substituted with 1-3 independently selected halogens, —OC(═O)(C₁-C₄ alkyl) optionally substituted with 1-3 independently selected halogens, or —(CH₂)_{K}C(═O)—(C₁-C₄ alkyl) optionally substituted with 1-3 independently selected halogens,
or
R^{TA} is

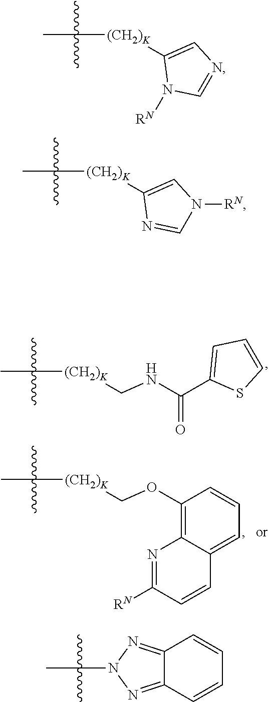

optionally substituted with 1-3 C₁-C₃ alkyl groups optionally substituted with 1-3 independently selected halogens,
or
R^{TA} is

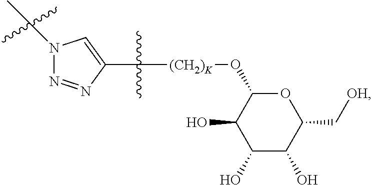

wherein each —(CH₂)_{K} group is optionally substituted with 1-4 C₁-C₃ alkyl groups optionally substituted with 1-3 fluoro groups or 1-2 hydroxyl groups.

In certain embodiments, the ASGPRBM group comprises the structure:

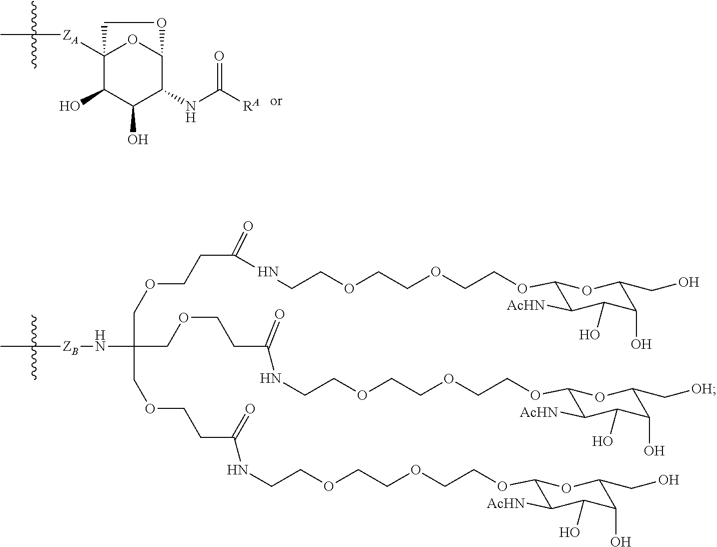

wherein:
R^{A} is C₁-C₃ alkyl optionally substituted with 1-5 independently selected halogens;
Z_{A} is —(CH₂)_{IM}-, —O—(CH₂)_{IM}, —S—(CH₂)_{IM}-, —NR^{M}—(CH₂)_{IM}-, —C(═O)—(CH₂)_{IM}-, a PEG group containing from 1 to 8 ethylene glycol residues, or —C(O)(CH₂)_{IM}NR^{M}-;
Z_{B} is absent, —(CH₂)_{IM}-, —C(═O)—(CH₂)_{IM}-, or —C(═O)(CH₂)_{IM}—NR^{M}—;
R^{M} is H or C₁-C₃ alkyl optionally substituted with 1-2 hydroxyl groups; and
each occurrence of IM is independently 0, 1, 2, 3, 4, 5, or 6.

In certain embodiments, R^{A} is methyl or ethyl, either of which is optionally substituted with 1-3 fluorines.

In certain embodiments, Z_{A} is a PEG group containing from 1 to 4 ethylene glycol residues.

In certain embodiments, the ASGPRBM group comprises one of the following (Mamidyala, et al., 2012, J. Am. Chem. Soc. 134:1978-1981):

3

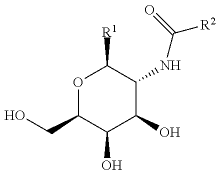

4

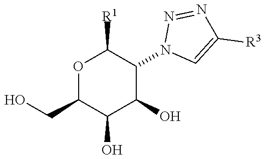

| | R¹ | # | R² |
|---|---|---|---|
| a | Me | 1 | Me |
| b | 4-OMePh | 2 | n-C₃H₇ |
| | | 3 | i-Pr |
| | | 4 | t-Bu |
| | | 5 | CCl₃ |
| | | 6 | CF₃ |
| | | 7 | Ot-Bu |
| | | 8 | CH₂CO₂H |
| | | 9 | CH₂NH₂ |
| | | 10 | CH₂CF₃ |
| | | 11 | 2-furyl |
| | | 12 | Ph |
| | | 13 | 4-OMePh |
| | | 14 | 3-OMePh |
| | | 15 | 4-CNPh |
| | | 16 | 5-pyridyl |
| | | 17 | 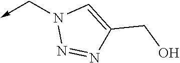 |
| | | 1 | Ph |
| | | 2 | 4-OMePh |
| | | 3 | 3-OMePh |
| | | 4 | 3-NH₂Ph |
| | | 5 | 4-NMe₂Ph |
| | | 6 | 2-pyridyl |
| | | 7 | 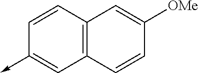 |
| | | 8 | 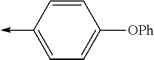 |
| | | 9 | 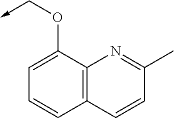 |
| | | 10 | 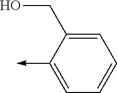 |
| | | 11 | 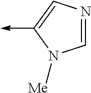 |
| | | 12 | 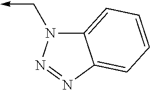 |
| | | 13 | 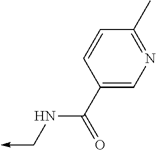 |
| | | 14 | 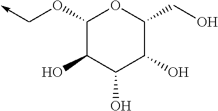 |
| | | 15 | CH₂CH |
| | | 16 | CH₂CH₂ |
| | | 17 | CH₂NHMe |
| | | 18 | CH₂NMe₂ |
| | | 19 | CO₂H |
| | | 20 | CH₂NHCOPh |
| | | 21 | CH₂NHCOMe |

6a-j

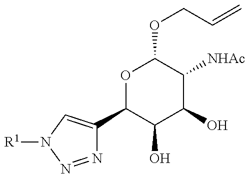

| | R¹ |
|---|---|
| a | Me |
| b | 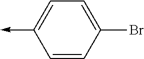 |
| c | 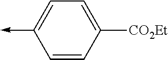 |
| d | 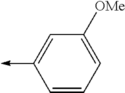 |
| e | 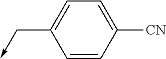 |
| f | 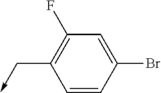 |
| g | 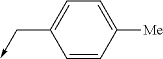 |
| h | 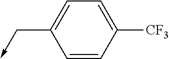 |
| i | 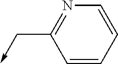 |
| j | 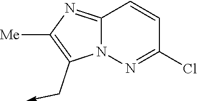 |

8a-j

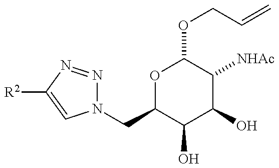

| | R² |
|---|---|
| a | CH₂OH |
| b | 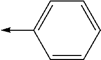 |
| c | 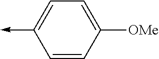 |
| d | 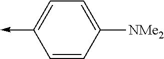 |
| e | 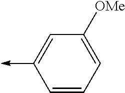 |
| f | 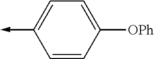 |
| g | 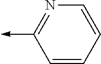 |
| h | 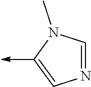 |
| i | 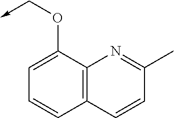 |
| j | 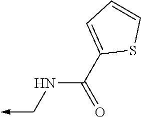 |

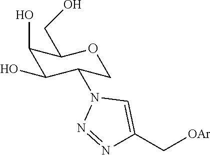

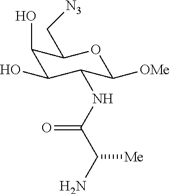 [SMILES: COC1OC(CN=[N+]=[N-])C(O)C(O)C1NC(=O)[C@H](C)N]

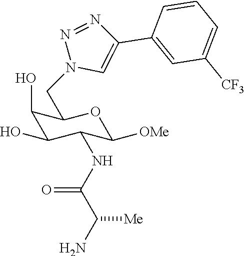 [SMILES: COC1OC(Cn2cc(-c3cccc(C(F)(F)F)c3)nn2)C(O)C(O)C1NC(=O)[C@H](C)N]

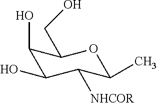

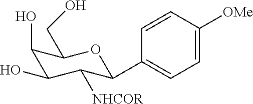

R=CH₃, CF₃, or CH₂CF₃;

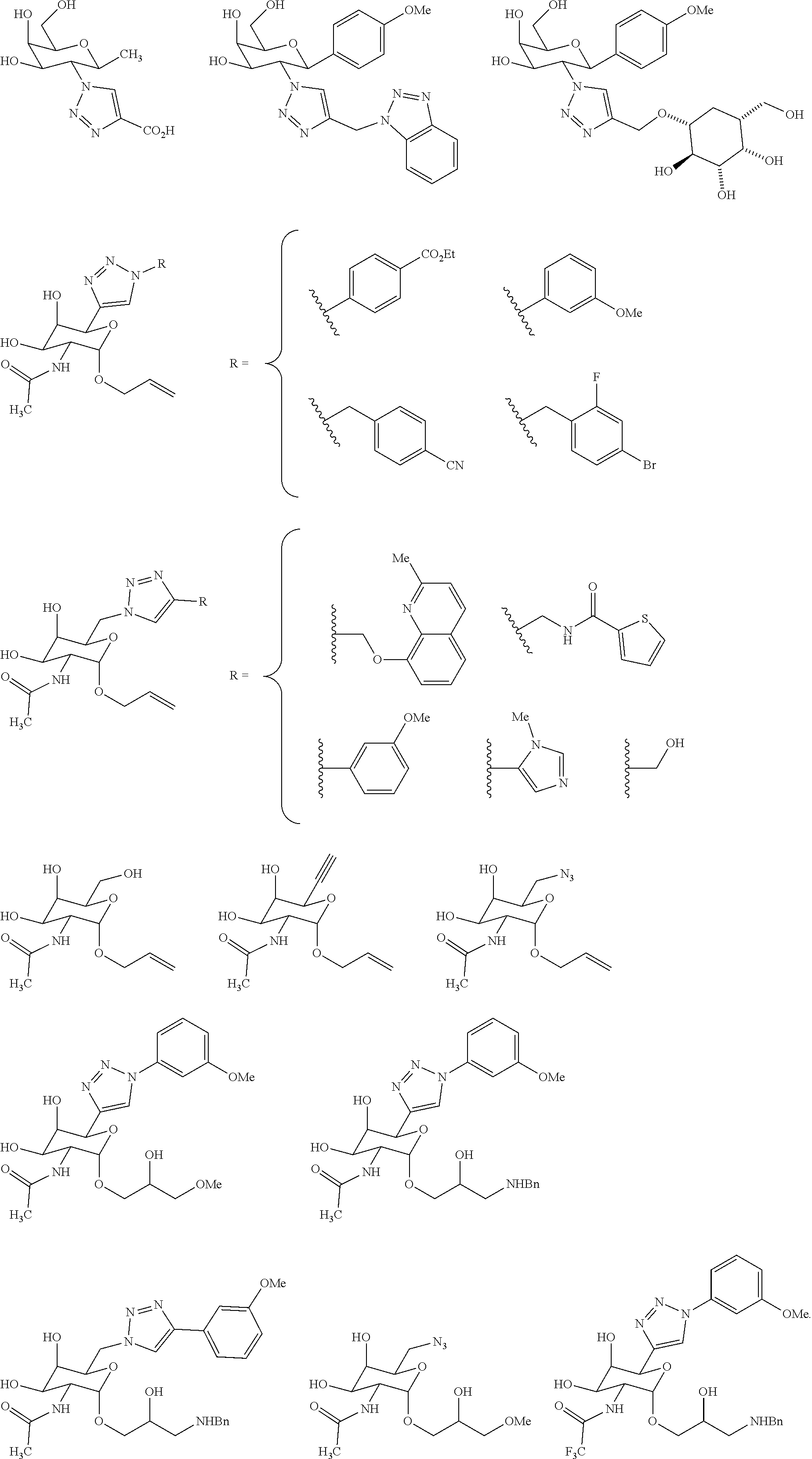

In certain embodiments, the ASGPRBM group comprises one of the following (Sanhueza, et al., 2017, J. Am. Chem. Soc. 139:3528-3536):

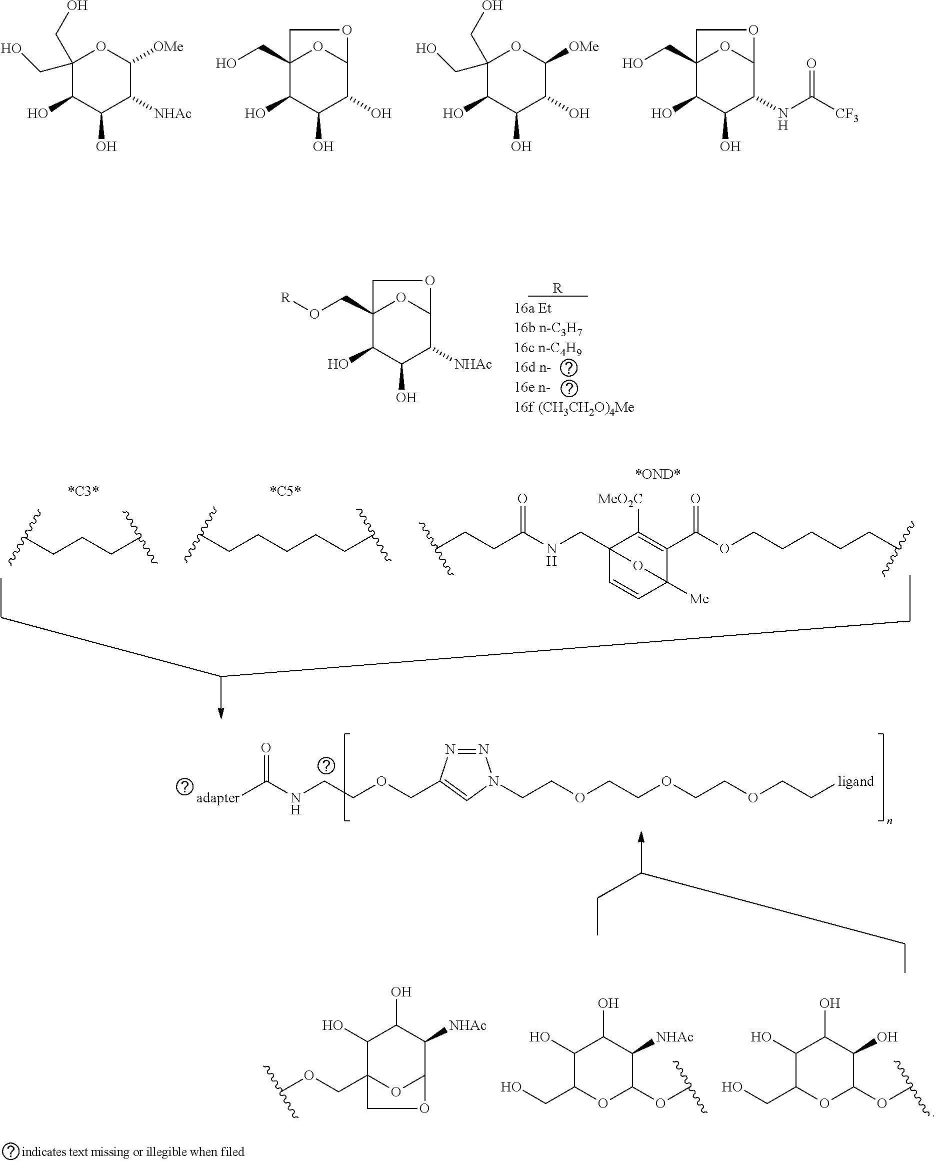

### Linker & CON

In certain embodiments, the Linker is a polyethylene glycol containing linker having 1-12 ethylene glycol residues.

In certain embodiments, the Linker comprises the structure: —CH₂CH₂(OCH₂CH₂)ₘOCH₂-, —(CH₂)ₘCH₂-, -[N(R^{a})—CH(R^{b})(C═O)]ₘ-, or a polypropylene glycol or polypropylene-co-polyethylene glycol group containing 1-100 alkylene glycol units;
wherein each R^{a} is independently H, C₁-C₃ alkyl, or C₁-C₆ alkanol, or combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
wherein m is an integer ranging from 1 to 15.

In certain embodiments, the Linker comprises the structure —[N(R′—(CH₂)₁₋₁₅—C(═O)]-, wherein R′ is H or a C₁-C₃ alkyl optionally substituted with 1-2 hydroxyl groups, and m is an integer ranging from 1 to 100.

In certain embodiments, the Linker comprises the structure

-Z-D-Z′-,

wherein:
Z and Z′ are each independently a bond, —(CH₂)ᵢ—O—, —(CH₂)ᵢ—S—, —(CH₂)ᵢ—N(R)-,

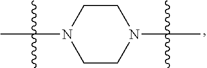 [SMILES: *N1CCN(*)CC1]

—(CH₂)ᵢ—C(R²)═C(R²)-(cis or trans), —(CH₂)ᵢ-≡-, or -Y-C(═O)—Y—;
each R is independently H, C₁-C₃ alkyl, or C₁-C₆ alkanol;
each R² is independently H or C₁-C₃ alkyl;
each Y is independently a bond, O, S, or N(R),
each i is independently 0 to 100; in certain embodiments 0 to 75; in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 0, 1, 2, 3, 4 or 5;
D is a bond, —(CH₂)ᵢ-Y—C(═O)—Y—(CH₂)ᵢ-, —(CH₂)_{m′}-, or -[(CH₂)ₘ-Xₗ)]ⱼ-, with the proviso that Z, Z′, and D are not each simultaneously bonds;
X¹ is O, S, or N(R);
j is an integer ranging from 1 to 100; in certain embodiments 1 to 75; in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 1, 2, 3, 4 or 5;
m′ is an integer ranging from 1 to 100; in certain embodiments 1 to 75; in certain embodiments 1 to 60; in certain embodiments 1 to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments I to 6; in certain embodiments 1, 2, 3, 4 or 5;
n is an integer ranging from 1 to 100; in certain embodiments 1 to 75; in certain embodiments 1 to 60; in certain embodiments I to 55; in certain embodiments 1 to 50; in certain embodiments 1 to 45; in certain embodiments 1 to 40; in certain embodiments 2 to 35; in certain embodiments 3 to 30; in certain embodiments 1 to 15; in certain embodiments 1 to 10; in certain embodiments 1 to 8; in certain embodiments 1 to 6; in certain embodiments 1, 2, 3, 4 or 5.

In certain embodiments, the Linker comprises a structure:

—CH₂—(OCH₂CH₂)ₙ—CH₂—, —(CH₂CH₂O)ₙ—CH₂CH₂—, or —(CH₂CH₂CH₂O)ₙ—,

wherein each n and n′ is independently an integer ranging from 1 to 25; in certain embodiments 1 to 15; in certain embodiments 1 to 12; in certain embodiments 2 to 11; in certain embodiments 2 to 10; in certain embodiments 2 to 8; in certain embodiments 2 to 6; in certain embodiments 2 to 5; in certain embodiments 2 to 4; in certain embodiments 2 or 3; in certain embodiments 1, 2, 3, 4, 5, 6, 7, or 8.

In certain embodiments, the Linker comprises a structure:

-PEG-CON-PEG-

wherein each PEG is independently a polyethylene glycol group containing from 1-12 ethylene glycol residues and CON is a triazole group

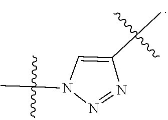 [SMILES: *c1cn(*)nn1]

In certain embodiments, the CON comprises a structure: N H

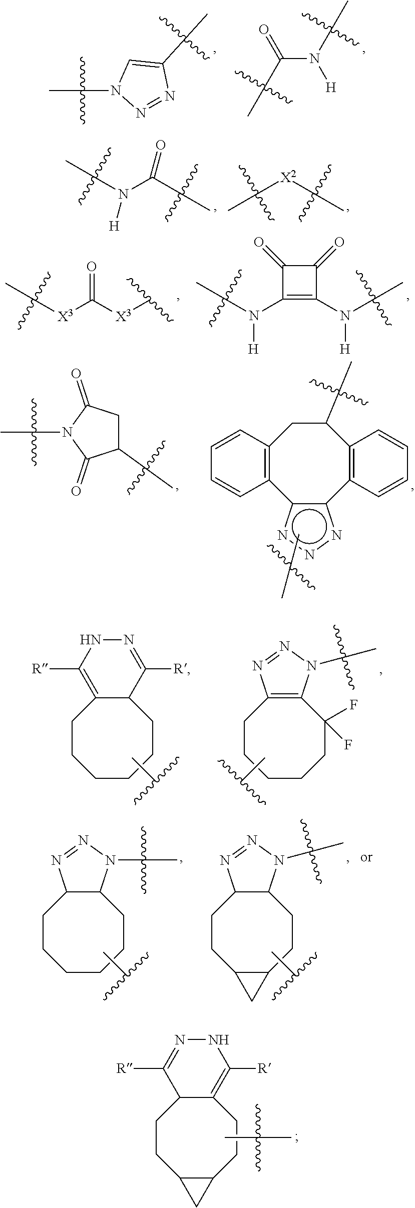

wherein R′ and R″ are each independently H, methyl, or a bond.

In certain embodiments, the CON comprises a diamide structure:

—C(═O)—N(R¹)—(CH₂)_{n″}—N(R¹)C(═O)—,

—N(R¹)—C(═O)(CH₂)_{n″}—C(═O)N(R¹)—, or

—N(R¹)—C(═O)(CH₂)_{n″}—N(R¹)C(═O)—;

wherein each R¹ is independently H or C₁-C₃ alkyl, and n″ is independently an integer from 0 to 8, in certain embodiments 1 to 7, in certain embodiments 1, 2, 3, 4, 5 or 6.

In certain embodiments, the CON comprises a structure:

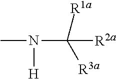

wherein:
R^{1a}, R^{2a} and R^{3a} are each independently H, —(CH₂)_{M1}-, —(CH₂)_{M2}C(═O)_{M3}(NR⁴)_{M3}—(CH₂)_{M2}-, —(CH₂)_{M2}(NR⁴)_{M3}C(O)_{M3}—(CH₂)_{M2}-, or —(CH₂)_{M2}O—(CH₂)_{M1}—C(O)NR⁴—, with the proviso that R^{1a}, R^{2a} and R^{3a} are not simultaneously H;
each M1 is independently 1, 2, 3, or 4; in certain embodiments, 1 or 2;
each M2 is independently 0, 1, 2, 3, or 4; in certain embodiments, 0, 1 or 2;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0.

In certain embodiments, the CON comprises a structure:

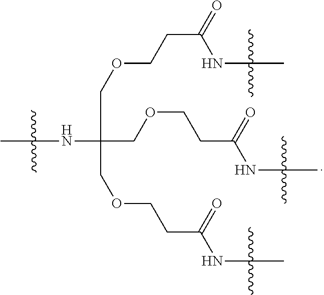 [SMILES: *NC(=O)CCOCC(COCCC(=O)N*)(COCCC(=O)N*)N*]

### Protein Binders

Any Protein binder that binds to a protein of interest (which in certain embodiments is a circulating protein) is useful within formula (I) and formula (Ia) of the present disclosure. In certain non-limiting embodiments, the binder is a small molecule. In certain non-limiting embodiments, the binder is a peptide and/or polypeptide.

The Protein binder can be incorporated within the compounds of formula (I) and/or formula (Ia) using any methods known in the art and/or any techniques described or illustrated herein. For example, the Protein binder can be attached to a Linker and/or CON using amide coupling, ester coupling, nucleophilic displacement, electrophilic displacement, radical coupling, or any other synthetic method known in the art. The attachment position of the Protein binder should be such that the attached Protein binder in formula (I) or formula (Ia) can still bind to the protein of interest. It is within the standard experimentation expected from, and known to, one skilled in the art to contemplate the mode of binding of the Protein binder to the protein of interest and identify potential sites of attachment on the Protein binder, and/or attach the Protein binder to a CON and/or linker and ascertain whether such attachment disturbs binding of the Protein binder to the protein of interest.

In certain embodiments, the Protein binder is an antibody, such as, but not limited to, a monoclonal antibody. The antibody of interest can be incorporated within the compounds of formula (I) or formula (Ta) using any methods known in the art and/or any techniques described or illustrated herein. For example, the antibody can be attached to a Linker and/or CON through a carboxylic acid group on the antibody's surface, using for example amide or ester formation chemistry. For example, the antibody can be attached to a Linker and/or CON through an amine group on the antibody's surface, using for example amide formation chemistry. For example, the antibody can be attached to a Linker and/or CON through a thiol group on the antibody's surface, using for example nucleophilic substitution chemistry. In that case, the surface cysteine residue can exist in the wild-type form of the antibody and/or can be introduced by mutation, using for example site-directed mutagenesis. The Linker and/or CON useful within the disclosure can be any linker known in the art, as long as the presence of the linker does not significantly disturb the antibody's ability to bind to the protein of interest.

In certain embodiments, the Protein binder is a polypeptide. The polypeptide of interest can be incorporated within the compounds of formula (I) or formula (Ia) using any methods known in the art and/or any techniques described or illustrated herein. For example, the polypeptide can be attached to a Linker and/or CON through its C-terminus and/or its N-terminus, using for example amide or ester formation chemistry. For example, the polypeptide can be attached to a Linker and/or CON through any intermediate residue using for example amide or ester formation chemistry and/or nucleophilic displacement chemistry (for example, if the polypeptide has a thiol residue). The polypeptide can be synthesized by standard Fmoc-SPPS. Introduction of a linker at either the N- or C-terminus followed by a functional handle (N₃, alkyne, and so forth) allows simple ligation to a targeting domain.

The Protein binders that are protein-based, such as antibodies, polypeptides, and the like, can be synthesized by various methods well known in the field, such as expression in E. coli for those not requiring post-translational modification (PTM) or in mammalian culture for those that do require PTM. These binding proteins can be made into bifunctional proteins by introduction of an unnatural amino acid tag for ligation (N₃, alkyne, and so forth) followed by reaction with the corresponding targeting domain, or by many other well-known bioorthogonal reactions for specific tagging of proteins.

As will be understood by one skilled in the art, any Protein binder that may recognize and specifically bind to the protein of interest is useful in the present disclosure. The disclosure should not be construed to be limited to any one type of Protein binder, either known or heretofore unknown, provided that the Protein binder can specifically bind to the protein of interest, and prevent or minimize biological activity of the protein of interest.

In certain embodiments, the protein of interest is CD40L. In certain embodiments, the Protein binder that binds to CD40L comprises the following (wherein the wavy lines indicate potential non-limiting points of attachment to REAG within contemplated compounds of the disclosure):

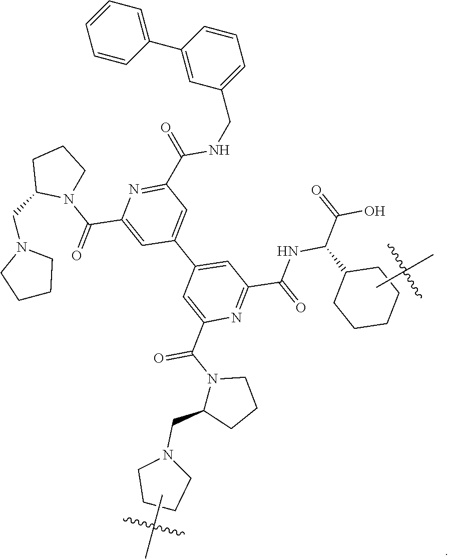

In certain embodiments, the protein of interest is PCSK9. In certain embodiments, the Protein binder that binds to PCSK9 comprises the following (wherein the peptide C-terminus is optionally amidated, and wherein the wavy lines indicate potential non-limiting points of attachment to REAG within contemplated compounds of the disclosure):

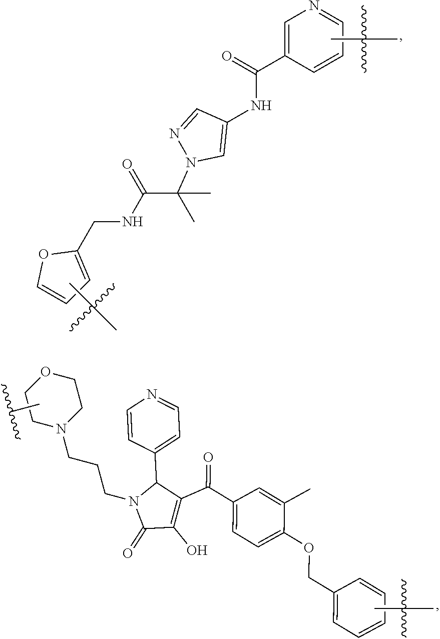

[symbol] NH-TVFTSWEEYLDWV-X (SEQ ID NO:66), wherein X=OH or NH₂.

In certain embodiments, the protein of interest is PCSK9. In certain embodiments, the Protein binder that binds to PCSK9 comprises any binder recited in WO2018/057409. In certain embodiments, the Protein binder comprises any of the following (wherein the wavy lines indicate potential non-limiting points of attachment to REAG within contemplated compounds of the disclosure)

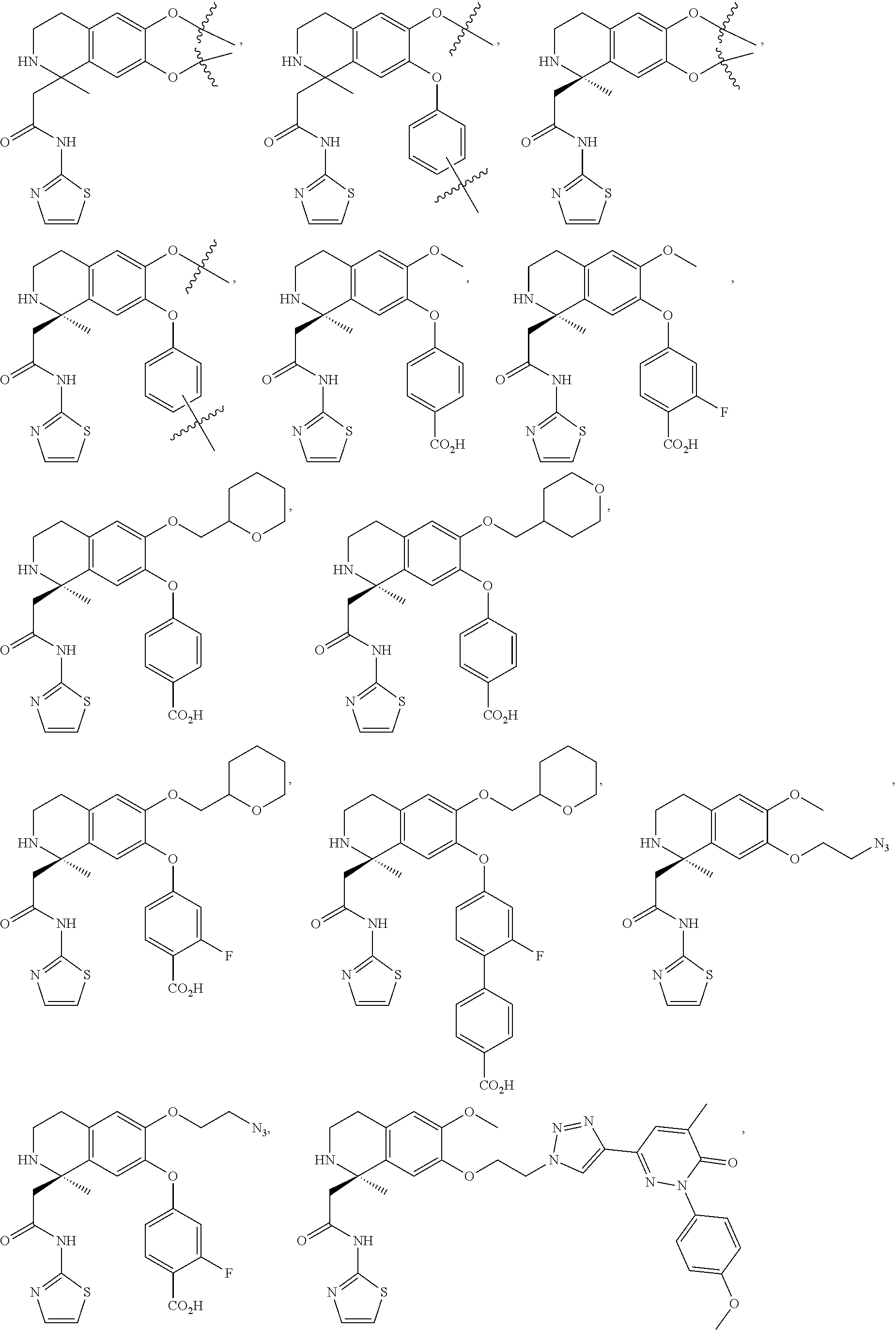

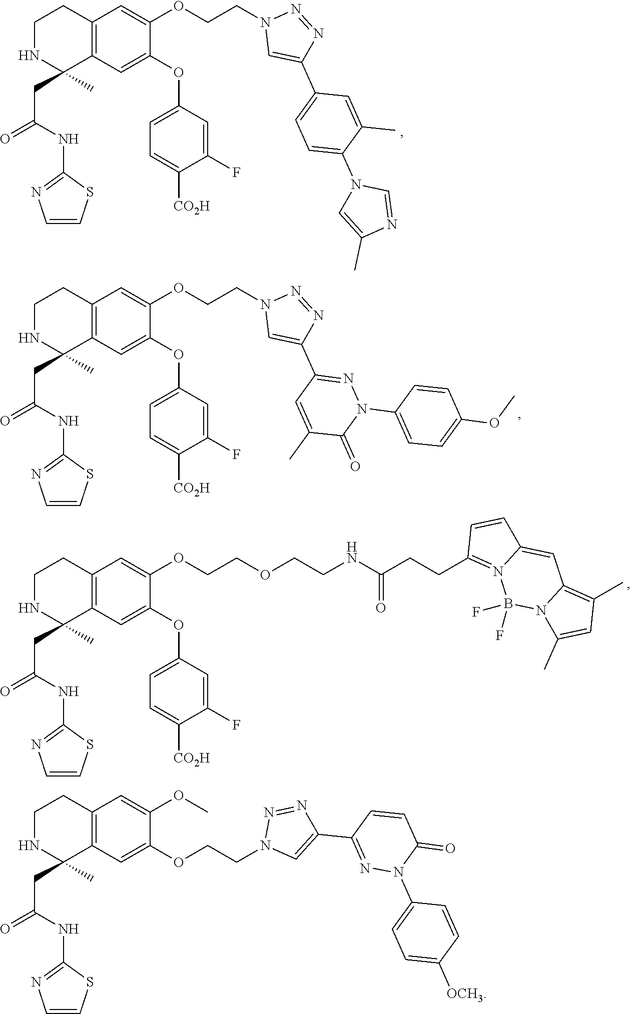

In certain embodiments, the protein of interest is VEGF. In certain embodiments, the Protein binder that binds to VEGF comprises the following (wherein the peptide C-terminus is optionally amidated, and wherein the wavy lines indicate potential non-limiting points of attachment to REAG within contemplated compounds of the disclosure):
[symbol] NH-VEPNCDIHVMWEWECFERL-X (SEQ ID NO:67), wherein X=OH or NH₂,

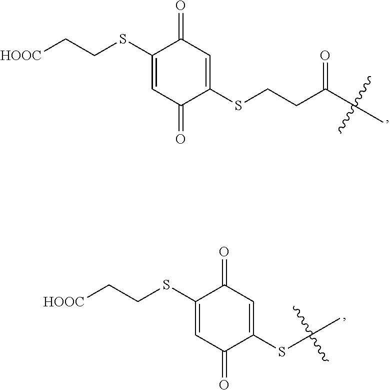 [SMILES: *C(=O)CCSC1=CC(=O)C(SCCC(=O)O)=CC1=O ; *SC1=CC(=O)C(SCCC(=O)O)=CC1=O]

In certain embodiments, the protein of interest is TGF-beta. In certain embodiments, the Protein binder that binds to TGF-beta comprises the following (wherein the peptide C-terminus is optionally amidated, and wherein the wavy lines indicate potential non-limiting points of attachment to REAG within contemplated compounds of the disclosure):
[symbol] NH-KRFKQDGGC-X (SEQ ID NO:68), wherein X=OH or NH₂

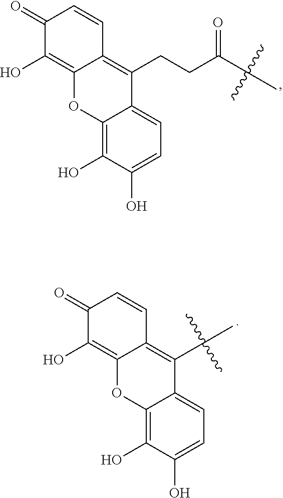 [SMILES: *C(=O)CCc1c2ccc(=O)c(O)c-2oc2c(O)c(O)ccc12 ; *c1c2ccc(=O)c(O)c-2oc2c(O)c(O)ccc12]

In certain embodiments, the protein of interest is TSP-1. In certain embodiments, the Protein binder that binds to TSP-1 comprises the following (wherein the peptide C-terminus is optionally amidated, and wherein the wavy lines indicate potential non-limiting points of attachment to REAG within contemplated compounds of the disclosure):
[symbol] NH-RGQILSKLRL-X (SEQ ID NO:69), wherein X=OH or NH₂

In certain embodiments, the protein of interest is soluble uPAR. In certain embodiments, the Protein binder that binds to uPAR comprises the following (wherein the wavy lines indicate potential non-limiting points of attachment to REAG within contemplated compounds of the disclosure):

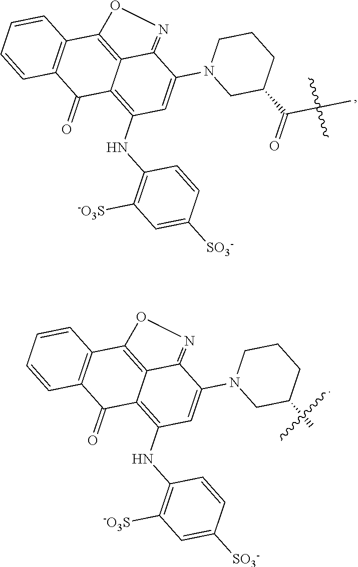

In certain embodiments, the protein of interest is soluble PSMA. In certain embodiments, the Protein binder that binds to PSMA comprises the following (wherein the wavy lines indicate potential non-limiting points of attachment to REAG within contemplated compounds of the disclosure):

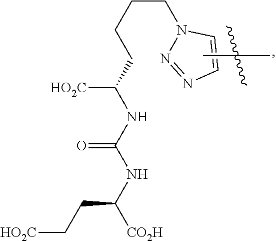

In certain embodiments, the protein of interest is IL-2. In certain embodiments, the Protein binder that binds to IL-2 comprises the following (wherein the wavy lines indicate potential non-limiting points of attachment to REAG within contemplated compounds of the disclosure):

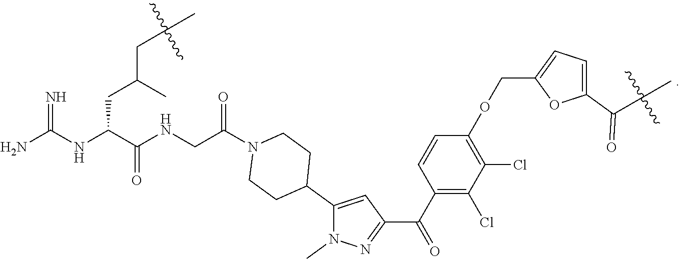 [SMILES: *CC(C)C[C@@H](NC(=N)N)C(=O)NCC(=O)N1CCC(c2cc(C(=O)c3ccc(OCc4ccc(C(*)=O)o4)c(Cl)c3Cl)nn2C)CC1]

In certain embodiments, the protein of interest is GP120. In certain embodiments, the Protein binder that binds to GP120 comprises the following (wherein the wavy lines indicate potential non-limiting points of attachment to REAG within contemplated compounds of the disclosure):

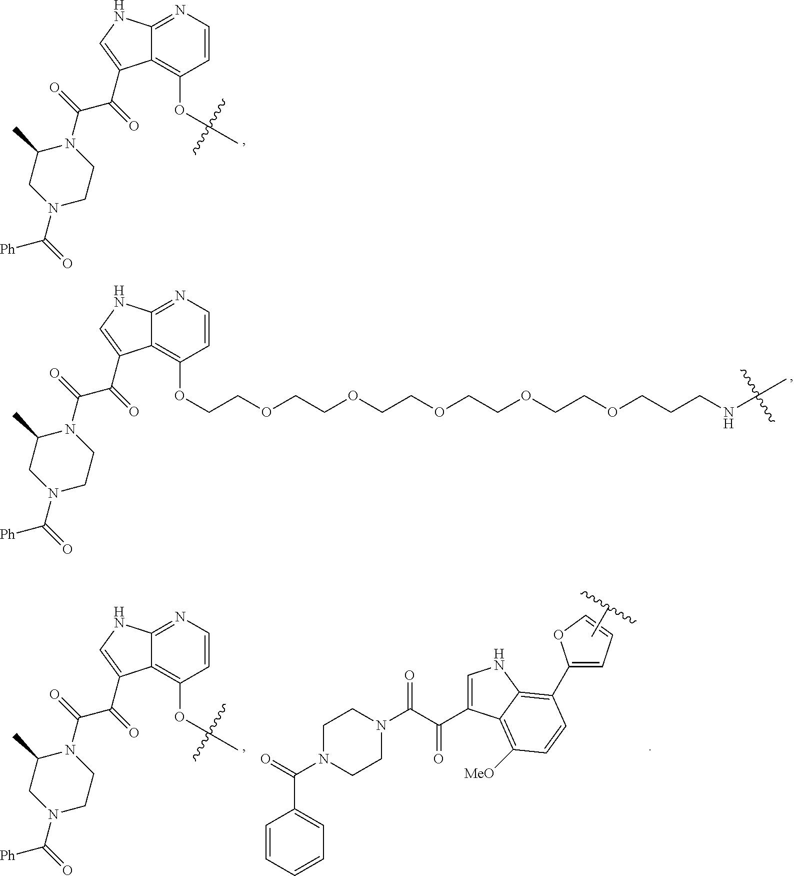

In certain embodiments, the protein of interest is MIF. In certain embodiments, the Protein binder that binds to MIF comprises the following (wherein the wavy lines indicate potential non-limiting points of attachment to REAG within contemplated compounds of the disclosure):

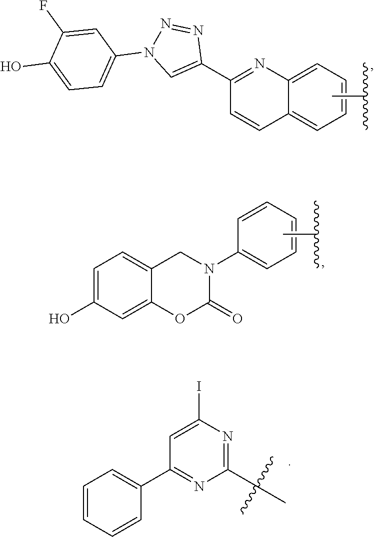

In certain embodiments, the protein of interest is IgA, as known in the art or described elsewhere herein. In certain embodiments, the Protein binder that binds to MIF comprises any peptide recited in Hatanaka, et al., 2012, J. Biol. Chem. 287:43126-43136, such as but not limited to:

(SEQ ID NO: 70)
STFCLLGQKDQSYCFTI
(SEQ ID NO: 71)
HMRCLHYKGRRVCFLL
(SEQ ID NO: 72)
KTMCLRYNHDKVCFRI
(SEQ ID NO: 73)
LVLCLVHRTSKHRKCFVI
(SEQ ID NO: 75)
A2-3a: SDVCLRYRGRPVCFQV
(SEQ ID NO: 76)
Opt-1: HMVCLAYRGRPVCFAL
(SEQ ID NO: 77)
Opt-2: HMVCLSYRGRPVCFSL
(SEQ ID NO: 78)
Opt-3: HQVCLSYRGRPVCFST
(SEQ ID NO: 79)
RDVCLRYRGRPVCFQV
(SEQ ID NO: 80)
HDVCLRYRGRPVCFQV
(SEQ ID NO: 81)
ADVCLRYRGRPVCFQV
(SEQ ID NO: 82)
SAVCLRYRGRPVCFQV
SEQ ID NO: 83)
SMVCLRYRGRPVCFQV
(SEQ ID NO: 84)
SDRCLRYRGRPVCFQV
(SEQ ID NO: 85)
SDACLRYRGRPVCFQV
(SEQ ID NO: 86)
SDVCARYRGRPVCFOV
(SEQ ID NO: 87)
SDVCLNYRGRPVCFQV
(SEQ ID NO: 88)
SDVCLHYRGRPVCFQV
(SEQ ID NO: 89)
SDVCLAYRGRPVCFQV
(SEQ ID NO: 90)
SDVCLRARGRPVCFQV
(SEQ ID NO: 91)
SDVCLRYAGRPVCFQV
(SEQ ID NO: 92)
SDVCLRYRARPVCFQV
(SEQ ID NO: 93)
SDVCLRYRGSPVCFQV
(SEQ ID NO: 94)
SDVCLRYRGAPVCFQV
(SEQ ID NO: 95)
SDVCLRYRGRRVCFQV
(SEQ ID NO: 96)
SDVCLRYRGRAVCFQV
(SEQ ID NO: 97)
SDVCLRYRGRPACFQV
(SEQ ID NO: 98)
SDVCLRYRGRPVCRQV
(SEQ ID NO: 99)
SDVCLRYRGRPVCAQV
(SEQ ID NO: 100)
SDVCLRYRGRPVCFRV
(SEQ ID NO: 101)
SDVCLRYRGRPVCFLV
(SEQ ID NO: 102)
SDVCLRYRGRPVCFAV
(SEQ ID NO: 103)
SDVCLRYRGRPVCFQW
(SEQ ID NO: 104)
SDVCLRYRGRPVCFQL
(SEQ ID NO: 105)
SDVCLRYRGRPVCFQA

These peptides can be acyclic (as free thiols) or cyclized as oxidized thiols (disulfide bonds). Further, the disclosure contemplates incorporating these peptides in the compounds of the disclosure through N- and/or C-terminus conjugation.

In certain embodiments, the Protein binder that binds to IgA is any Fc-alpha receptor peptide mimetic recited in Heineke, et al., 2017, Eur. J. Immunol. 47:1835-1845, such as but not limited to:

### Linear Peptides:

(SEQ ID NO: 106)
GRYQCQYRIGHYRFRYSD
(SEQ ID NO: 107)
GRYQAQYRIGHYRFRYSD
(SEQ ID NO: 108)
GRYQCQYRIGHYRFRYSD

Cyclic peptides: CLIPS

(SEQ ID NO: 109)
CLIPS-CHYRFRC
SEQ ID NO: 110)
CLIPS-CRIGHYRFRC
(SEQ ID NO: 111)
CLIPS-YQACHYRFRC
(SEQ ID NO: 112)
CLIPS-RYQAQCRIGHYRFC
(SEQ ID NO: 113)
CLIPS-GRYQCQYRIGHYRFRYCD
(SEQ ID NO: 114)
CLIPS-GRYQACYRIGHYRFRCSD
(SEQ ID NO: 115)
CLIPS-GRYQAQCRIGHYRFCYSD

### Cyclic Peptides: Oxidated

(SEQ ID NO: 116)
RYQAQCRIGHYRFC
(SEQ ID NO: 117)
GRYQCQYRIGHYRFRYCD
(SEQ ID NO: 118)
GRYQACYRIGHYRFRCSD
(SEQ ID NO: 119)
GRYQAQCRIGHYRFCYSD

These peptides can be acyclic (as free thiols) or cyclized as oxidized thiols (disulfide bonds). Further, the disclosure contemplates incorporating these peptides in the compounds of the disclosure through N- and/or C-terminus conjugation.

CLIPS indicates cyclization of linear peptides via reaction of thiol-functionalities of the cysteines with a small rigid entity; this anchor reacts exclusively with thiols and attaches to the peptide via covalent bonds. Non-limiting examples of CLIPS cross-linkers contemplated in the present disclosure include:

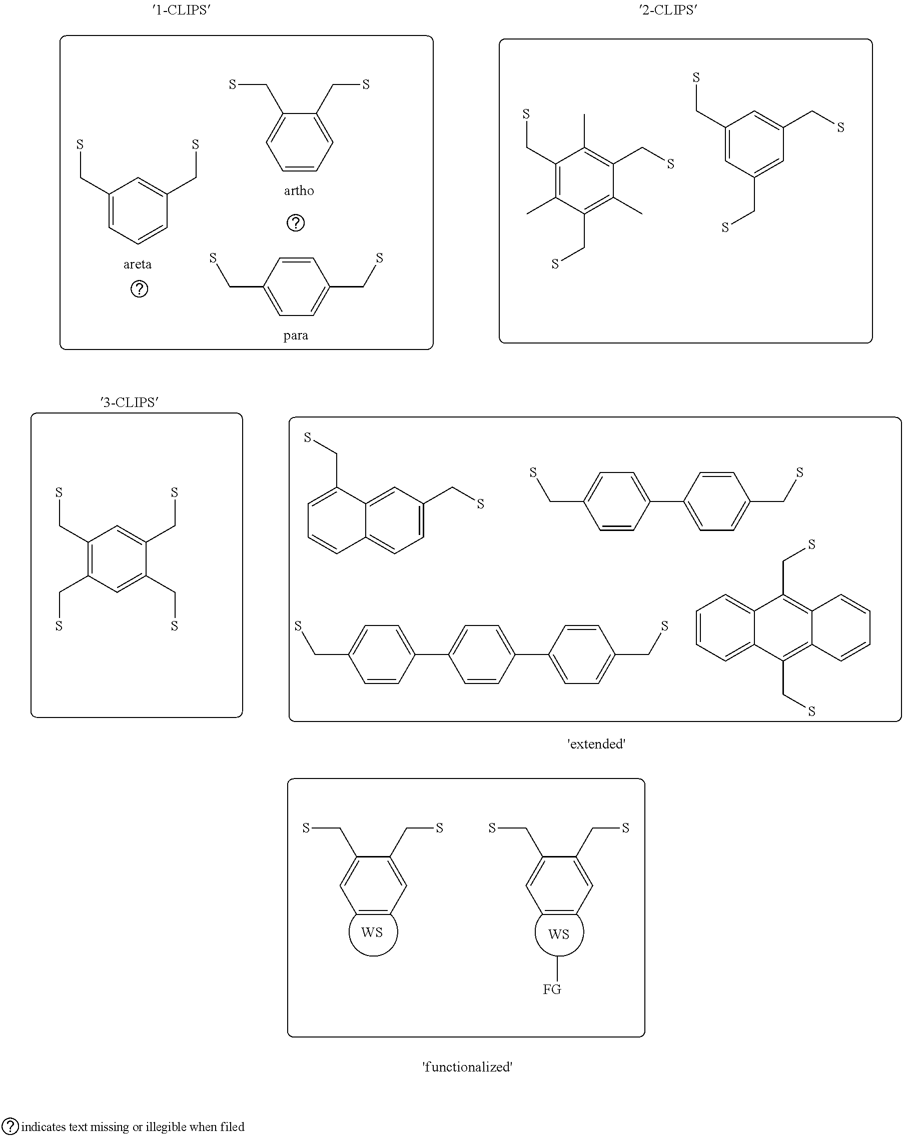

### TNF Binders

Any TNF binder that binds to TNF is useful within formula (II) and formula (IIa) of the present disclosure. In certain non-limiting embodiments, the binder is a small molecule. In certain non-limiting embodiments, the binder is a peptide and/or polypeptide.

The TNF binder can be incorporated within the compounds of formula (II) and formula (IIa) using any methods known in the art and/or any techniques described or illustrated herein. For example, the TNF binder can be attached to a Linker and/or CON using amide coupling, ester coupling, nucleophilic displacement, electrophilic displacement, radical coupling, or any other synthetic method known in the art. The attachment position of the TNF binder should be such that the attached TNF binder in formula (II) or formula (IIa) can still bind to TNF. It is within the standard experimentation expected from, and known to, one skilled in the art to contemplate the mode of binding of the TNF binder to TNF and identify potential sites of attachment on the TNF binder, and/or attach the TNF binder to a CON and/or linker and ascertain whether such attachment disturbs binding of the TNF binder to TNF.

In certain embodiments, the TNF binder is an antibody, such as, but not limited to, a monoclonal antibody. The antibody of interest can be incorporated within the compounds of formula (II) and formula (IIa) using any methods known in the art and/or any techniques described or illustrated herein. For example, the antibody can be attached to a Linker and/or CON through a carboxylic acid group on the antibody's surface, using for example amide or ester formation chemistry. For example, the antibody can be attached to a Linker and/or CON through an amine group on the antibody's surface, using for example amide formation chemistry. For example, the antibody can be attached to a Linker and/or CON through a thiol group on the antibody's surface, using for example nucleophilic substitution chemistry. In that case, the surface cysteine residue can exist in the wild-type form of the antibody and/or can be introduced by mutation, using for example site-directed mutagenesis. The Linker and/or CON useful within the disclosure can be any linker known in the art, as long as the presence of the linker does not significantly disturb the antibody's ability to bind to TNF.

In certain embodiments, the TNF binder is a polypeptide. The polypeptide of interest can be incorporated within the compounds of formula (II) and formula (IIa) using any methods known in the art and/or any techniques described or illustrated herein. For example, the polypeptide can be attached to a Linker and/or CON through its C-terminus and/or its N-terminus, using for example amide or ester formation chemistry. For example, the polypeptide can be attached to a Linker and/or CON through any intermediate residue using for example amide or ester formation chemistry and/or nucleophilic displacement chemistry (for example, if the polypeptide has a thiol residue). The polypeptide can be synthesized by standard Fmoc-SPPS. In certain embodiments, the C-terminus of the peptide is amidated. Introduction of a linker at either the N- or C-terminus followed by a functional handle (N₃, alkyne, and so forth) allows simple ligation to an ASGPR targeting domain. In a non-limiting example:

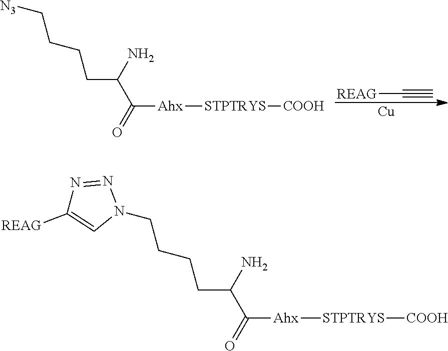

The TNF binders that are protein-based, such as antibodies, polypeptides, and the like, can be synthesized by various methods well known in the field, such as expression in E. coli for those not requiring post-translational modification or in mammalian culture for those that do require PTM. These binding proteins can be made into bifunctional proteins targeting TNF-ASGPR by introduction of an unnatural amino acid tag for ligation (N₃, alkyne, and so forth) followed by reaction with the corresponding ASGPR targeting domain, or by many other well-known bioorthogonal reactions for specific tagging of proteins.

As will be understood by one skilled in the art, any TNF binder that may recognize and specifically bind to TNF is useful in the present disclosure. The disclosure should not be construed to be limited to any one type of TNF binder, either known or heretofore unknown, provided that the TNF binder can specifically bind to TNF, and prevent or minimize biological activity of TNF.

In certain embodiments, the TNF binder comprises the polypeptide STPTRYS (SEQ ID NO:120) (Guangdong Yixue 2008, 29(1):55-57).

In certain embodiments, the TNF binder comprises the polypeptide CALWHWWHC SEQ ID NO:121) or C(T/S)WLHWWAC (SEQ ID NO:122) (Diyi Daxue Xuebao 2002, 22(7):597-599).

In certain embodiments, the TNF binder comprises any Tbab protein described in Zhu, et al., 2016, Protein Sci. 25:2066-2075.

In certain embodiments, the TNF binder comprises the polypeptide (L/M)HEL(Y/F)(L/M)X(W/Y/F) (SEQ ID NO:123), as described in Zhang, et al., 2003, Biochem. Biophys. Res. Commun. 310:1181-1187.

In certain embodiments, the TNF binder comprises one of the polypeptides:

| (SEQ ID NO: 124) | |
|---|---|
| DHPT-9: | D-DDDEK QLKER WYKRW LEYLD EFKKN |
| (SEQ ID NO: 125) | |
| DHPT-91: | D-TEEEK QLKEW WYKHW QEYLE EFKKN |

### (Yang, et al., 2019, FEBS Lett. 593:1292-1302).

In certain embodiments, the TNF binder comprises TNFR1 or TNFR2 (Yang & Yang, 2013, Fenxi Huaxue/Chinese J. Anal. Chem. 41:664-669).

In certain embodiments, the TNF binder comprises anticachexin C1 and/or C2 (Lian, et al., 2013, J. Am. Chem. Soc. 135:11990-11995).

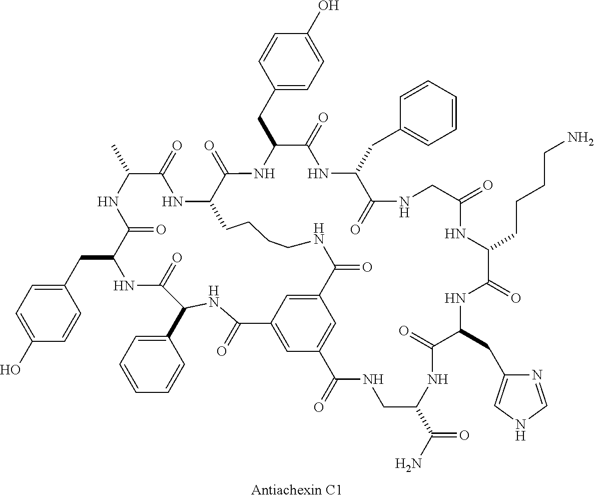

In certain embodiments, the TNF binder comprises adalimumab, infliximab, etanercept, golimumab, and/or certolizumab.

In certain embodiments, the TNF binder comprises the 29.2 kDa scFv identified in Safarpour, et al., 2018, Iran. J. Pharm. Res. 17:743-752.

In certain embodiments, the TNF binder comprises GACPPCLWQVLCGGSGSGSG (SEQ ID NO: 126) (which can be, in a non-limiting example, tris-bromomethyl mesitylene core sulfur linked; Luzi, et al., 2015, Protein Eng. Des. Sel. 28:45-52).

In certain embodiments, the TNF binder comprises any affibodies (˜60 amino acids) identified in Lofdahl, et al., 2009, N. Biotechnol. 26:251-259.

In certain embodiments, the TNF binder comprises any affibodies identified in Kronqvist, et al., 2008, Protein Eng. Des. Sel. 21:247-255.

In certain embodiments, the TNF binder comprises any affibodies identified in Jonsson, et al., 2009, Biotechnol. Appl. Biochem. 54:93-103.

In certain embodiments, the TNF binder comprises the bispecific albumin/TNF binding polypeptide identified in Nilvebrant, et al., 2011, PLoS One 6.

In certain embodiments, the TNF binder comprises the ubiquitin-based artificial binding protein identified in Hoffmann, et al., 2012, PLoS One 7:2-11.

In certain embodiments, the TNF binder comprises HIHDDLLRYYGW linear (SEQ ID NO:127) or tetra branched peptide (SEQ ID NO:128) identified in Brunetti, et al., 2014, Molecules 19:7255-7268.

In certain embodiments, the TNF binder comprises any TNF-α binding peptides (P51 and P52) identified in Alizadeh, et al., 2017, Eur. J. Pharm. Sci. 96:490-498.

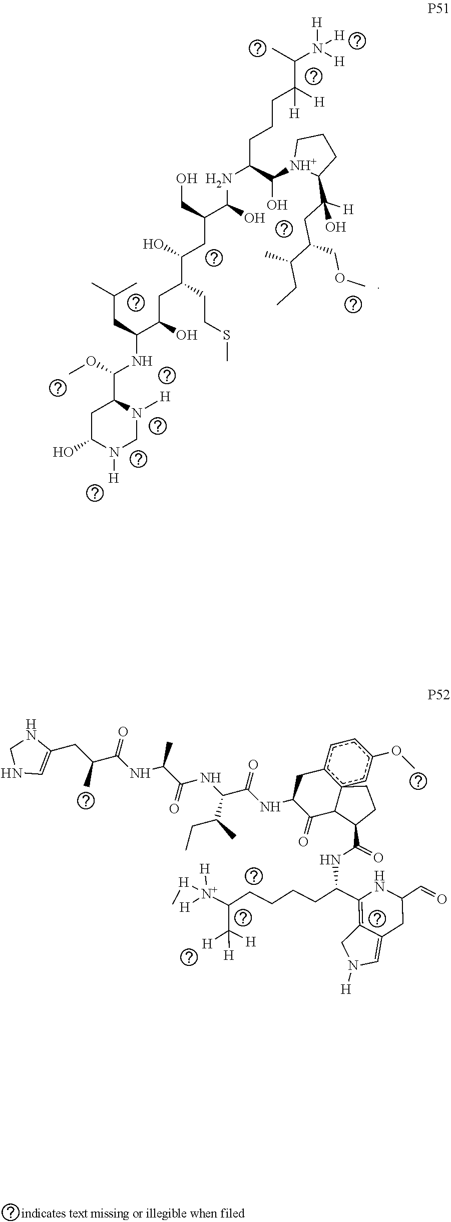

In certain embodiments, the TNF binder comprises the scFv antibody identified in Alizadeh, et al., 2015, Adv. Pharm. Bull. 5:661-666.

In certain embodiments, the TNF binder comprises any TNF binding peptide recited in WO 2006/053568 (such as but not limited to KRWSRYF (SEQ ID NO:129), which may in certain embodiments be polyvalent), which is incorporated herein in its entirety by reference.

In certain embodiments, the TNF binder comprises any TNF binding peptide recited in WO 2015/055597 (such as but not limited to HIHDDLLRYYGW (SEQ ID NO:127), which may in certain embodiments be polyvalent), which is incorporated herein in its entirety by reference.

In certain embodiments, the TNF binder comprises YCWSQYLCY (SEQ ID NO: 130) as identified in Arthritis & Rheumatism 2007, 56(4):1164-74.

In certain embodiments, the TNF binder comprises DFLPHYKNTSLGHRP (SEQ ID NO—131) as identified in Chirinos-Rojas, et al., 1998, J. Immunol. 161:5621-5626.

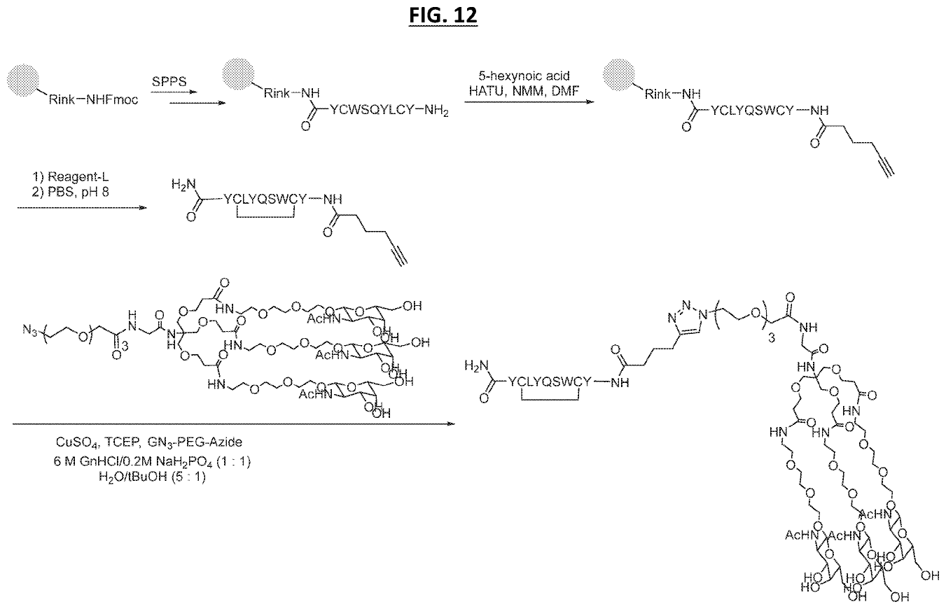

In certain embodiments, the TNF binder comprises YCLYQSWCY (SEQ ID NO: 132). In certain embodiments, the TNF binder is its reduced form (i.e., with an internal disulfide bond). In certain embodiments, the TNF binder is its oxidized form (i.e., without an internal disulfide bond). See FIG. 12 as a non-limiting example.

In certain embodiments, the TNF binder comprises one of the following:

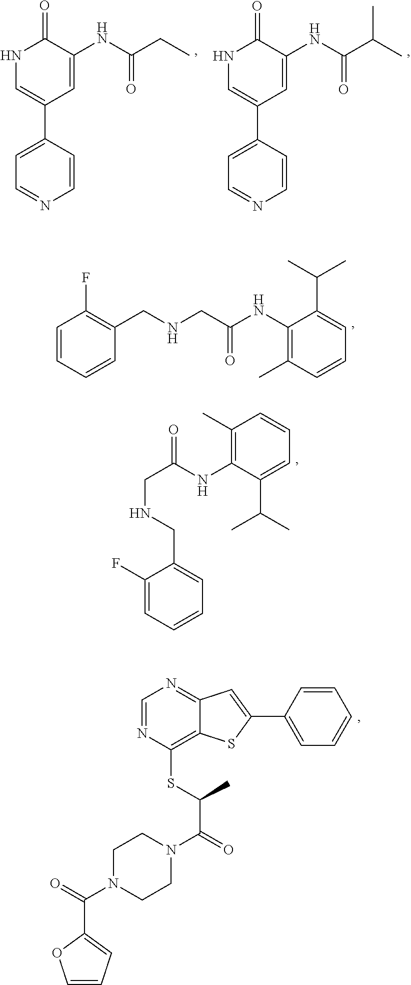

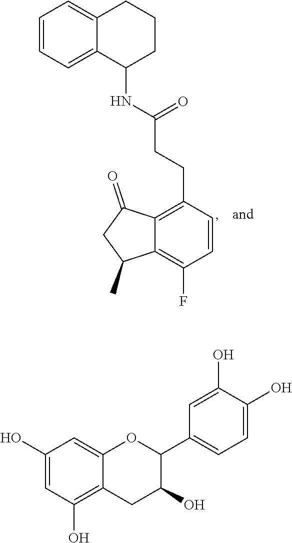 [SMILES: C[C@H]1CC(=O)c2c(CCC(=O)NC3CCCc4ccccc43)ccc(F)c21 ; Oc1cc(O)c2c(c1)OC(c1ccc(O)c(O)c1)[C@@H](O)C2]

(Zaka, et al., 2019, J. Biomol. Struct. Dyn. 37:2464-2476).

In certain embodiments, the TNF binder comprises one of the following:

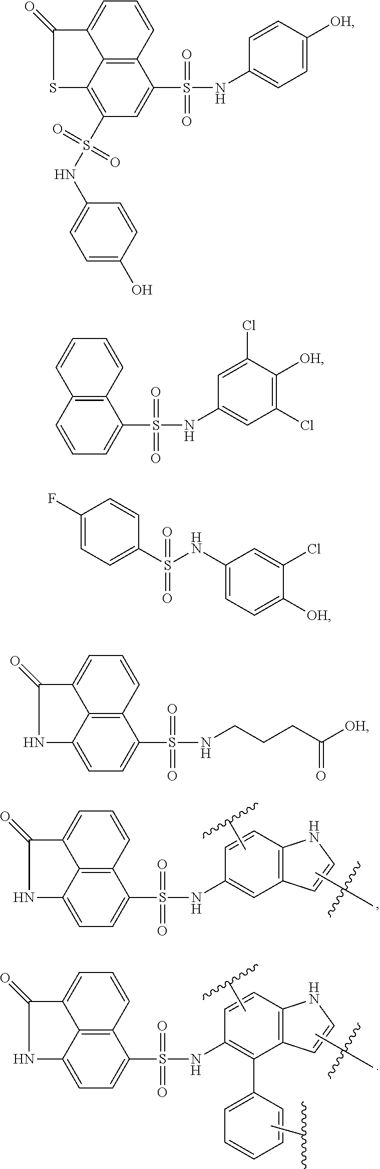

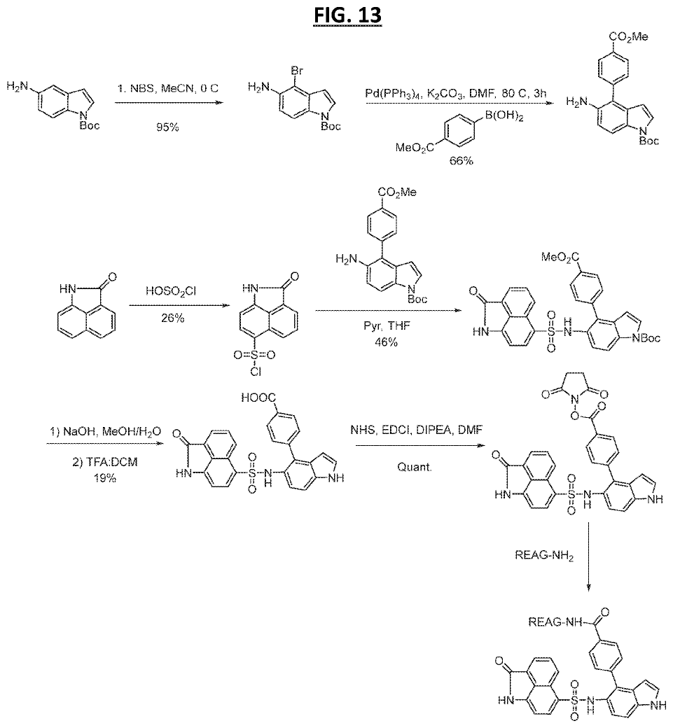

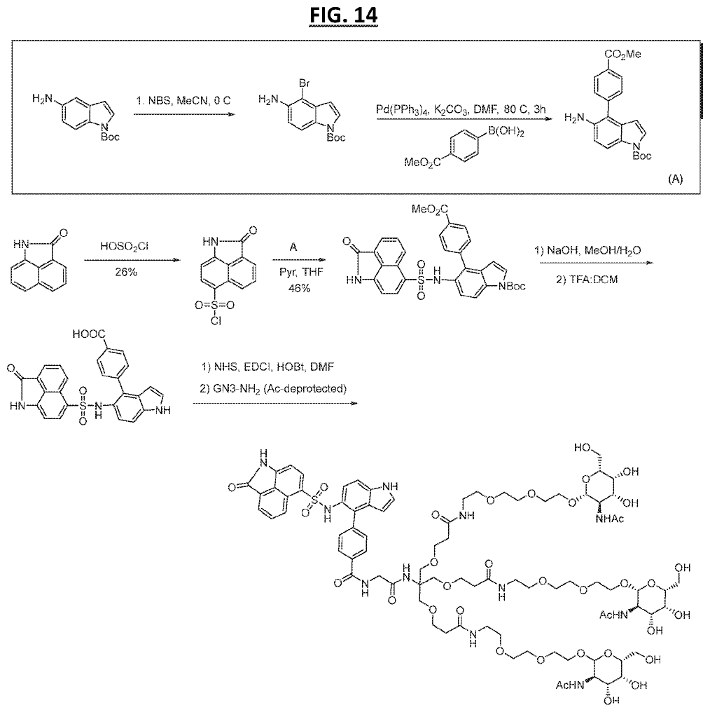

(Shen, et al., 2014, Eur. J. Med. Chem. 85:119-126). See FIG. 13 and FIG. 14 as non-limiting examples.

In certain embodiments, the TNF binder comprises:

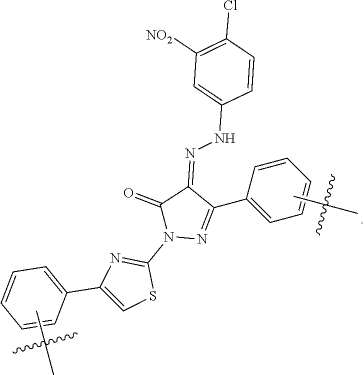

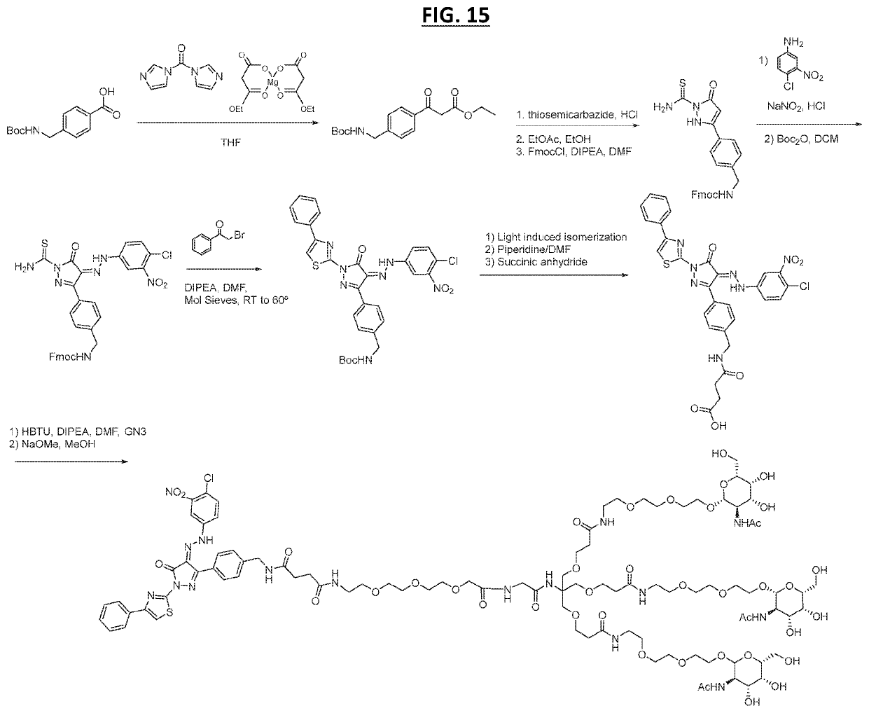

See FIG. 15 as a non-limiting example.

In certain embodiments, the TNF binder comprises:

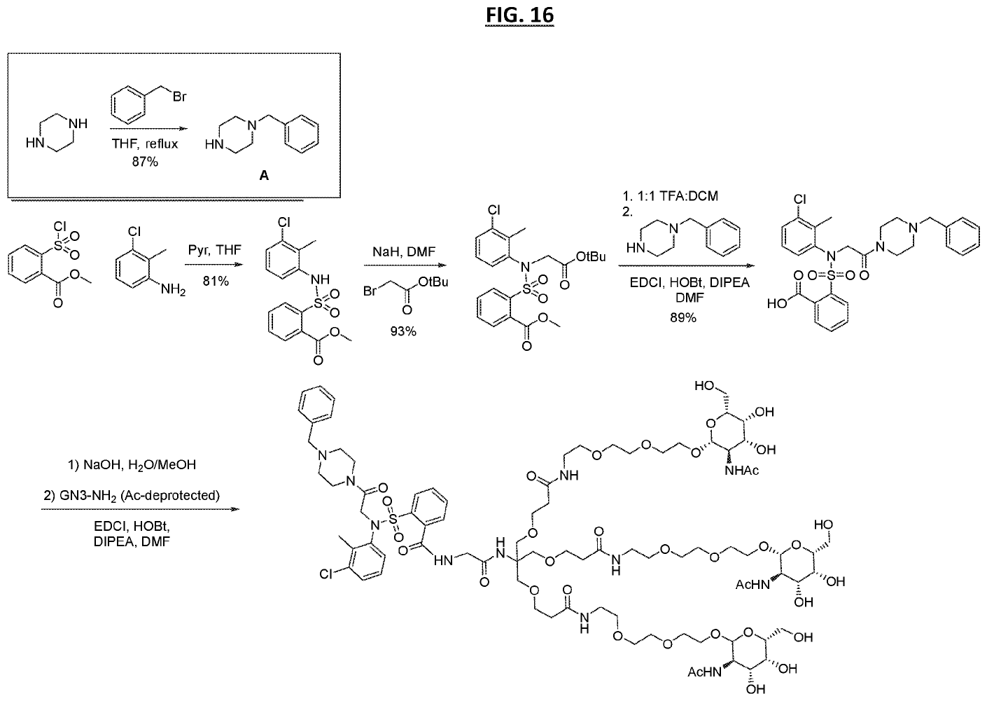

See FIG. 16 as a non-limiting example.

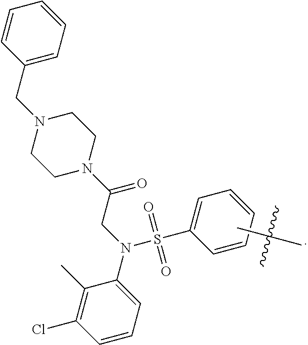

In certain embodiments, the TNF binder comprises:

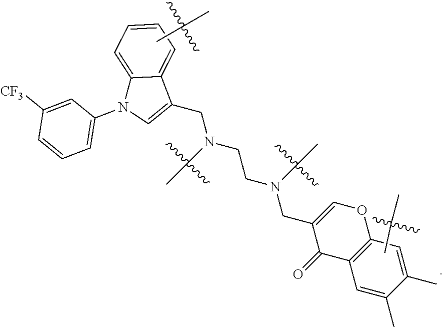

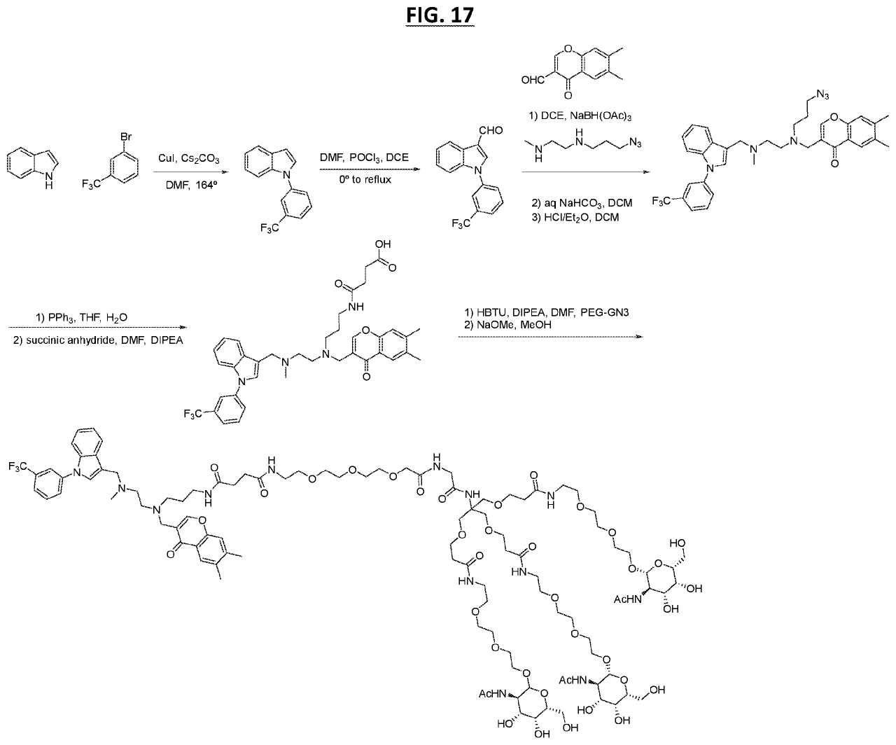

See FIG. 17 as a non-limiting example.

In certain embodiments, the TNF binder comprises one of the following (Saddala & Huang, 2019, J. Transl. Med. 17:1-16):

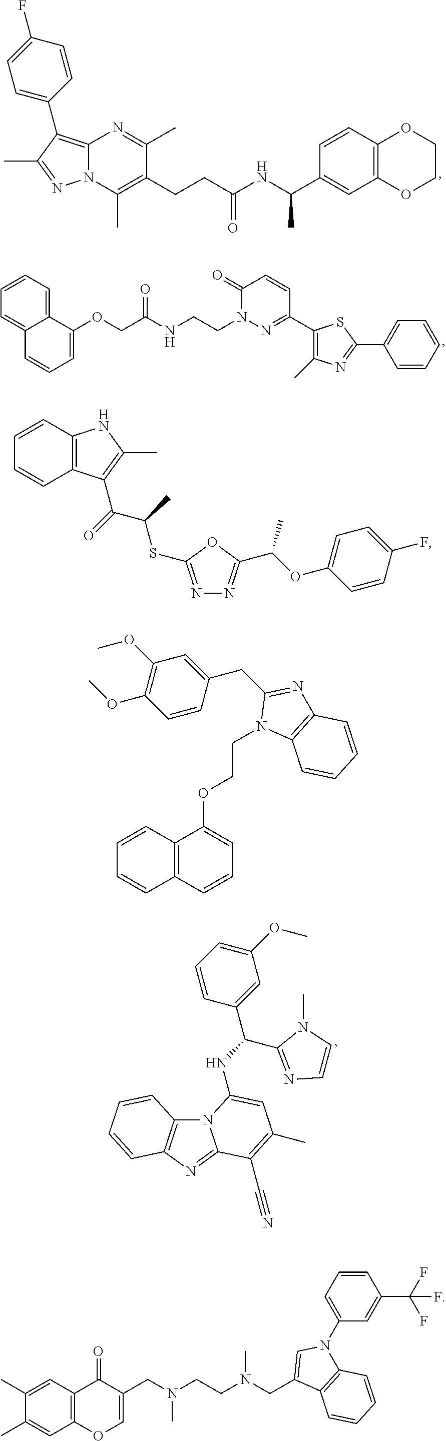 [SMILES: Cc1nc2c(-c3ccc(F)cc3)c(C)nn2c(C)c1CCC(=O)N[C@H](C)c1ccc2c(c1)OCCO2 ; Cc1nc(-c2ccccc2)sc1-c1ccc(=O)n(CCNC(=O)COc2cccc3ccccc23)n1 ; Cc1[nH]c2ccccc2c1C(=O)[C@@H](C)Sc1nnc([C@H](C)Oc2ccc(F)cc2)o1 ; COc1ccc(Cc2nc3ccccc3n2CCOc2cccc3ccccc23)cc1OC ; COc1cccc([C@@H](Nc2cc(C)c(C#N)c3nc4ccccc4n23)c2nccn2C)c1 ; Cc1cc2occ(CN(C)CCN(C)Cc3cn(-c4cccc(C(F)(F)F)c4)c4ccccc34)c(=O)c2cc1C]

In certain embodiments, the TNF binder comprises SPD-304 and analogs thereof (He, et al., 2005, Science 310:1022-1025; Papaneophytou, et al., 2015, Medchemcomm 6:1196-1209):

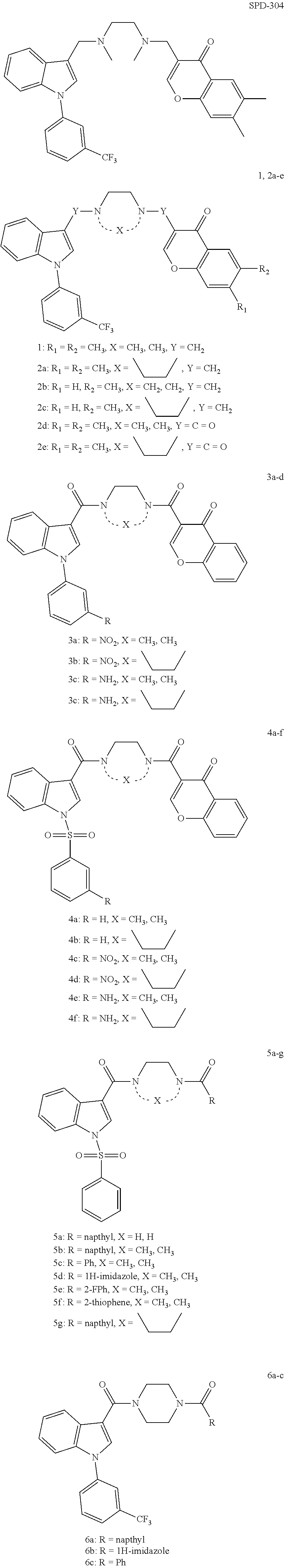

Non-limiting chemical schemes to prepare and derivatize these compound is provided herein:

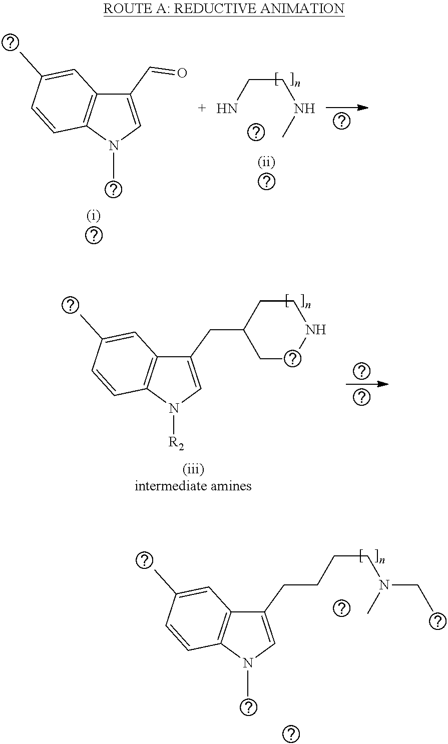

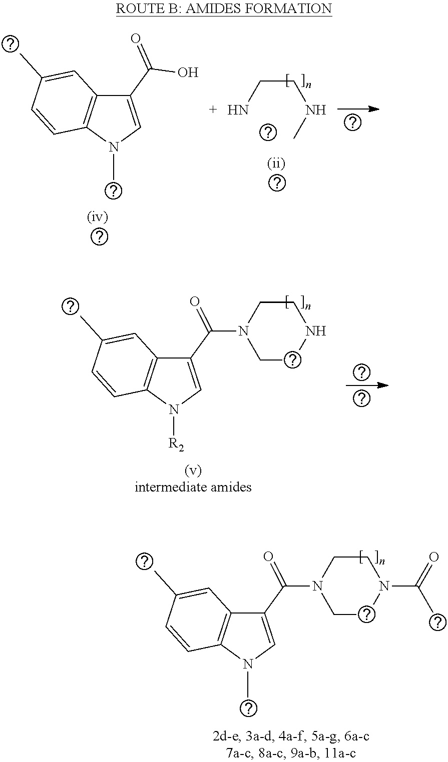

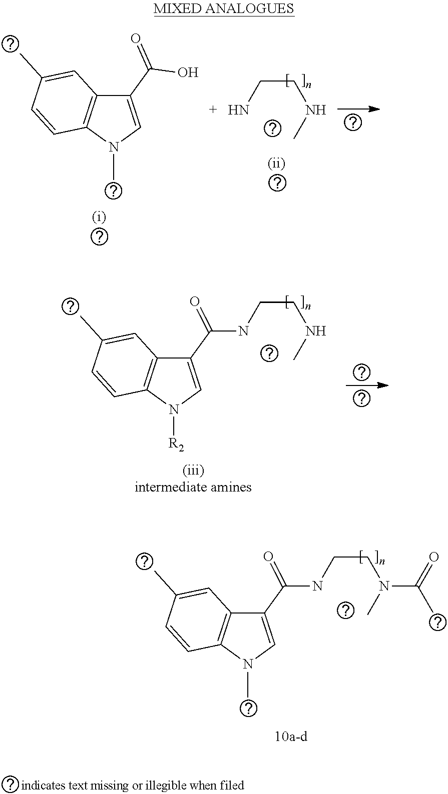

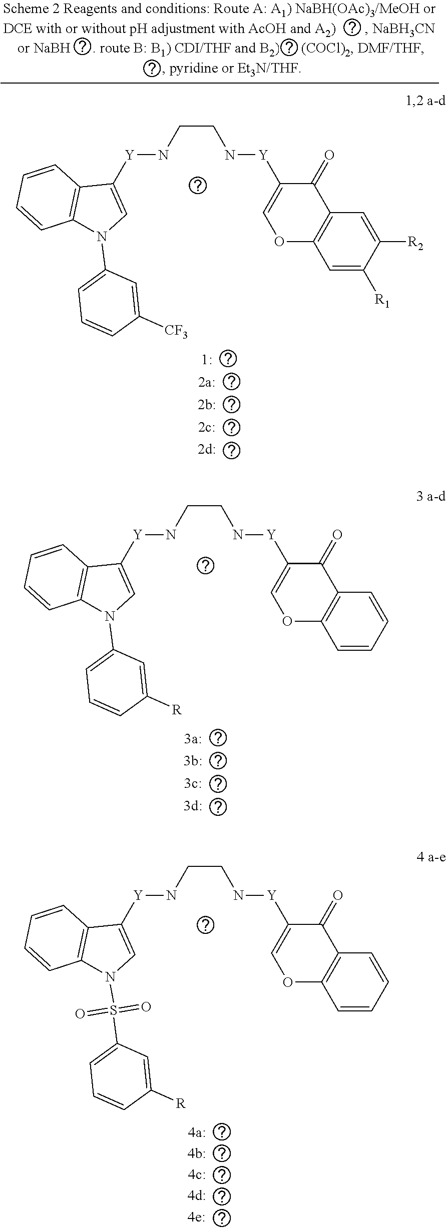

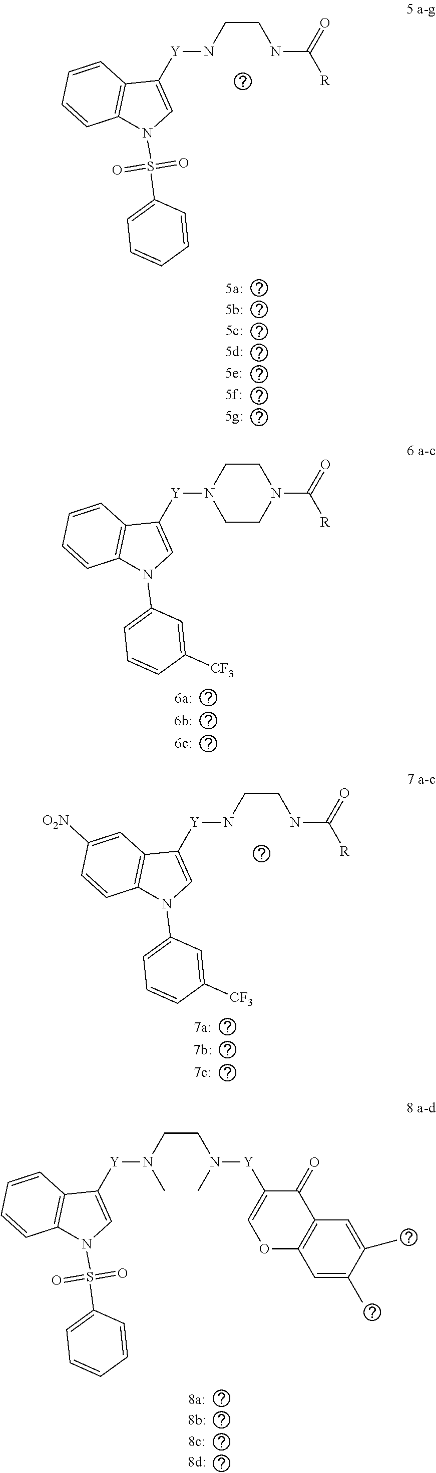

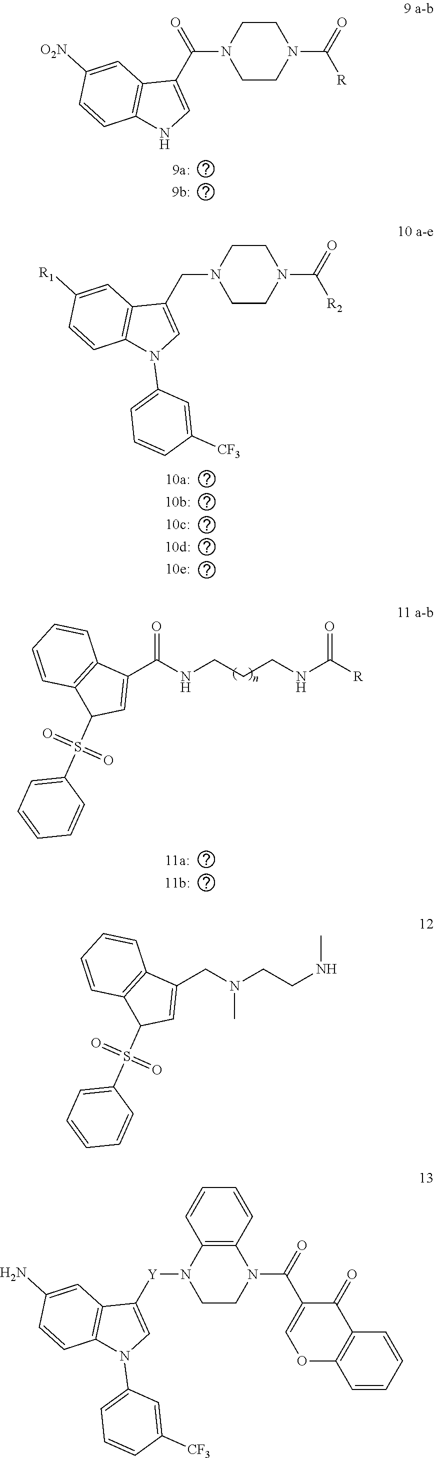

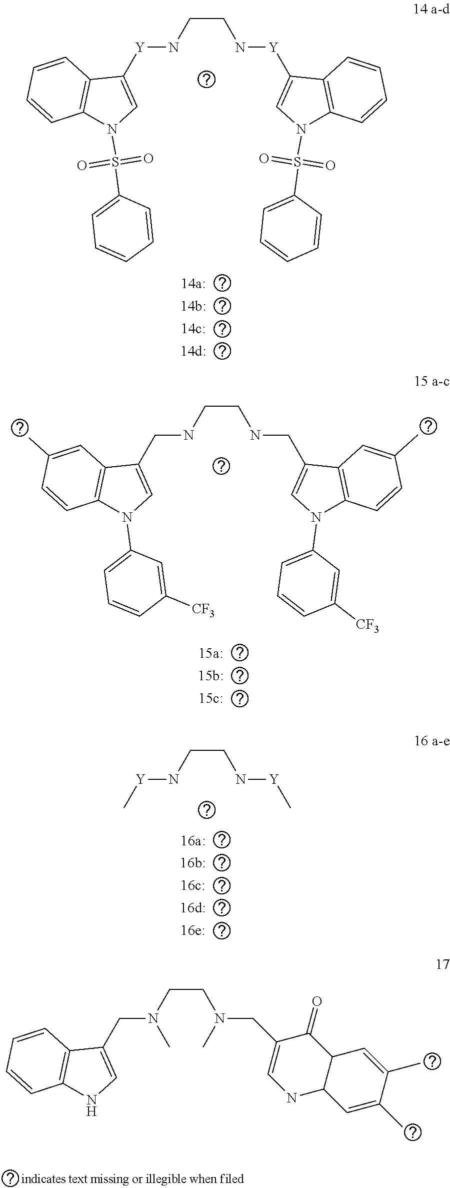

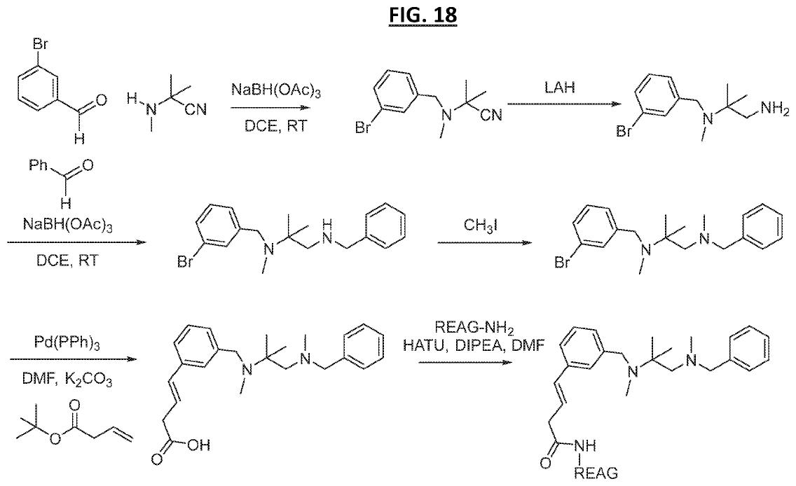

Structures of the 5S compounds studied (SPD304; compound I and 54 SPD304 analogues; compounds 2a-17) (Mettou, et al., 2018, SLAS Discov. 23:84-93). See FIG. 18 as a non-limiting example.

In certain embodiments, the TNF binder comprises a compound of formula (2a):

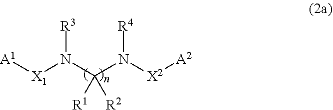

wherein:
A¹ and A² are independently a substituted or unsubstituted phenyl group, wherein the substituents comprise at least one of F, Cl, Br, I, OH, C₁-C₄ alkyl, C₁-C₄ alkyl substituted with at least one OH, C₁-C₄ fluoroalkyl (such as, but not limited to, CF₃), C₁-C₄ alkoxy, C₁-C₄ haloalkoxy, benzyloxy, and the following heterocyclic rings optionally substituted with at least one of F, Cl, Br, I. OH, C₁-C₄ alkyl, C₁-C₄ alkyl substituted with at least one OH, C₁-C₄ fluoroalkyl (such as, but not limited to, CF₃), C₁-C₄ alkoxy, and C₁-C₄ haloalkoxy (dotted lines indicate point of attachment):

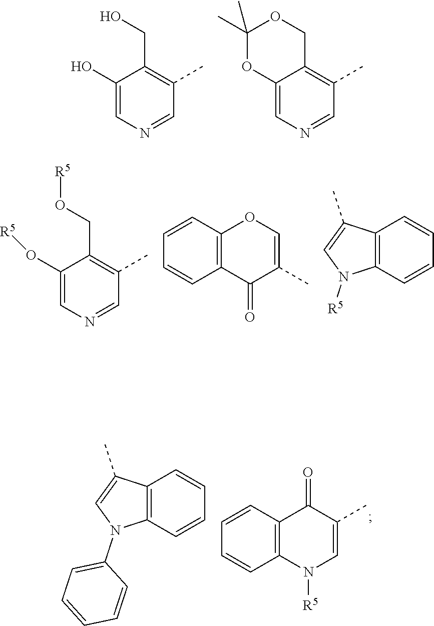

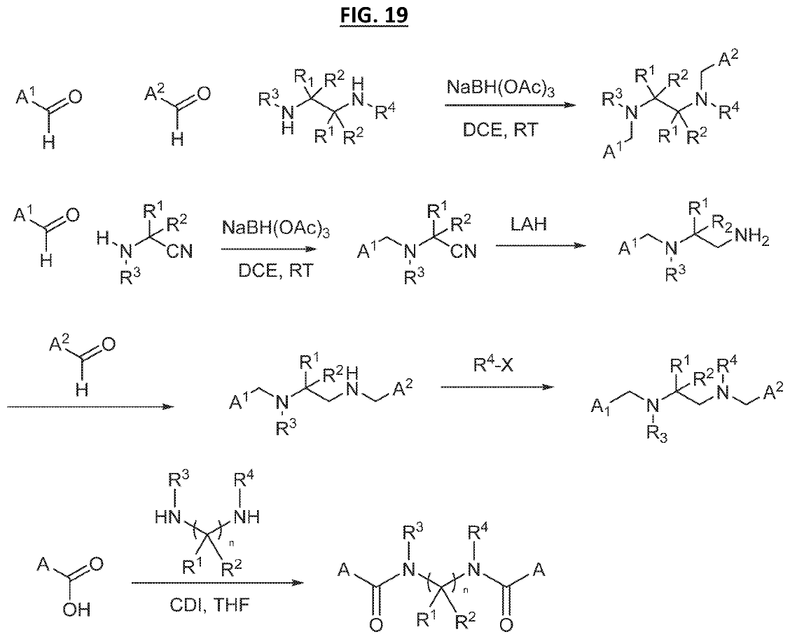

each R⁵ is independently hydrogen or optionally substituted C₁-C₄ alkyl;
R¹ and R² are independently hydrogen or optionally substituted C₁-C₄ alkyl;
X¹ and X² are independently carbonyl or CH₂;
n is 2, 3, or 4;
R³ and R⁴ are independently hydrogen or optionally substituted C₁-C₄ alkyl, or R³ and R⁴ can combine to form a heterocyclyl ring. See FIG. 19 as a non-limiting example.

For example, when A¹ and A² are 1-(3-(trifluoromethyl)phenyl)-1H-indole and 6,7-dimethyl-4H-chomen-4-one respectively, and X¹ and X² are both CH₂, R³ and R⁴ form a heterocyclyl ring, such as but not limited to a piperazinyl ring.

In certain embodiments, the TNF binder comprises the small molecule IA-14069.

In certain embodiments, the TNF binder comprises

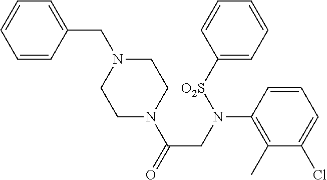 [SMILES: Cc1c(Cl)cccc1N(CC(=O)N1CCN(Cc2ccccc2)CC1)S(=O)(=O)c1ccccc1]

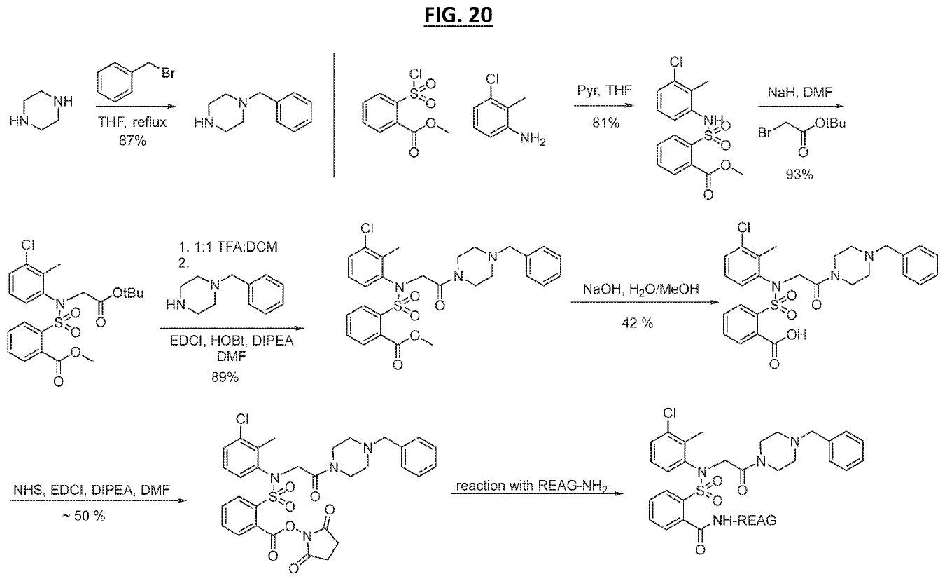

(Mouhsine, et al., 2017, Sci. Rep. 7:1-10 (2017). In certain embodiments, the Linker and/or Con can be attached the sulfonamido phenyl ring. See FIG. 20 as a non-limiting example.

In certain embodiments, the TNF binder comprises one of the following:

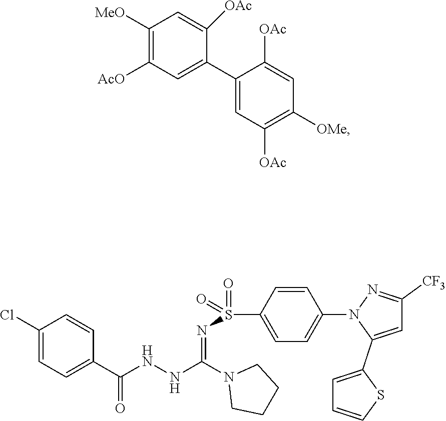

(Melagraki, et al., 2017, PLoS Comput. Biol. 13:1-27).

In certain embodiments, the TNF binder comprises one of the following:

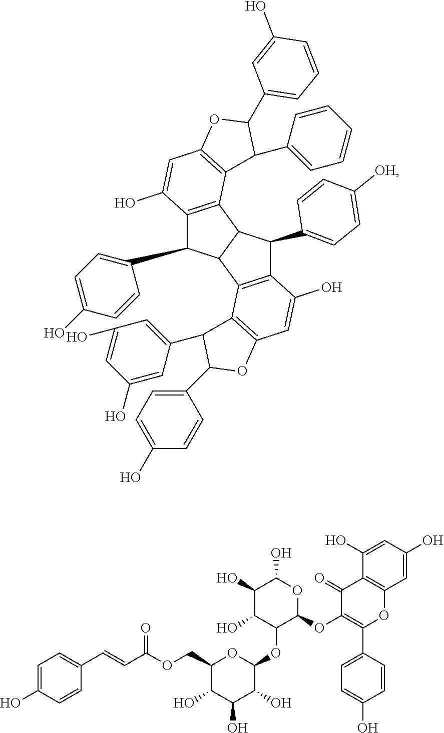 [SMILES: Oc1ccc(C2Oc3cc(O)c4c(c3C2c2cc(O)cc(O)c2)C2C(c3c5c(cc(O)c3[C@@H]2c2ccc(O)cc2)OC(c2cccc(O)c2)C5c2ccccc2)[C@H]4c2ccc(O)cc2)cc1 ; O=C(/C=C/c1ccc(O)cc1)OC[C@H]1O[C@@H](OC2[C@H](Oc3c(-c4ccc(O)cc4)oc4cc(O)cc(O)c4c3=O)O[C@@H](O)[C@H](O)[C@H]2O)[C@H](O)[C@@H](O)[C@@H]1O]

(Melagraki, et al. 2018, Front. Pharmacol. 9:1-12).

In certain embodiments, the TNF binder comprises

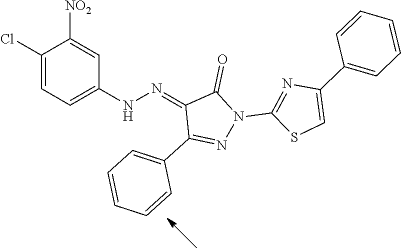 [SMILES: O=C1/C(=N/Nc2ccc(Cl)c([N+](=O)[O-])c2)C(c2ccccc2)=NN1c1nc(-c2ccccc2)cs1]

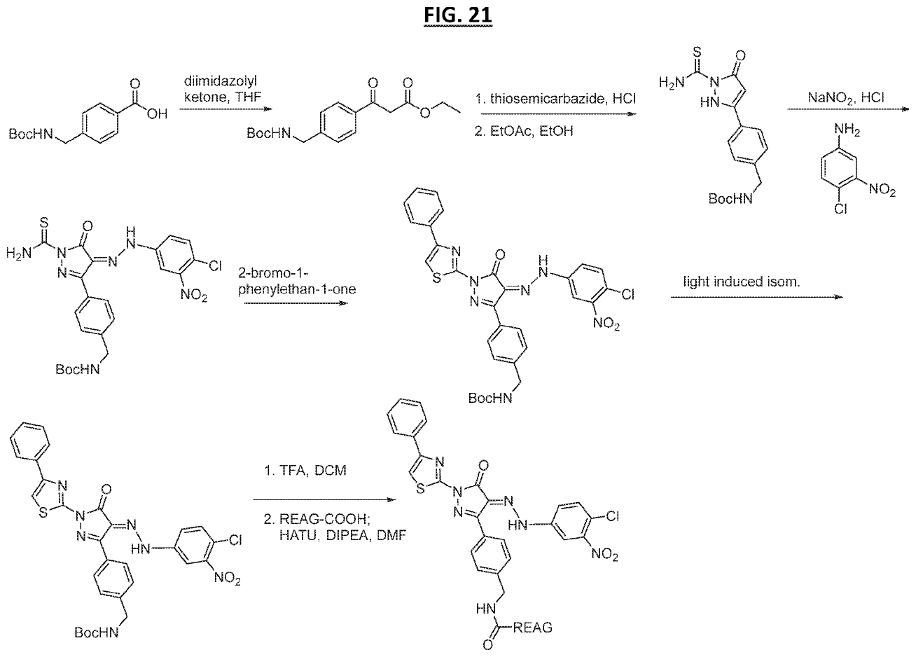

(Ma, et al., 2014, J. Biol. Chem. 289:12457-12466). In certain embodiments, the Linker and/or CON can be attached to the phenyl group marked with an arrow. See FIG. 21 as a non-limiting example.

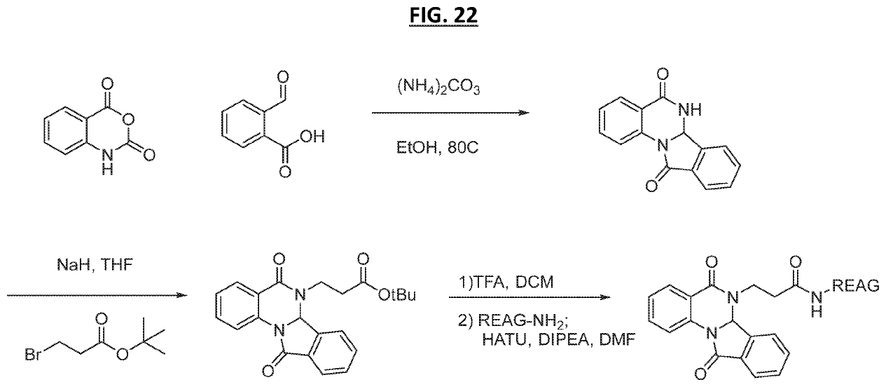

In certain embodiments, the TNF binder comprises one of the following:

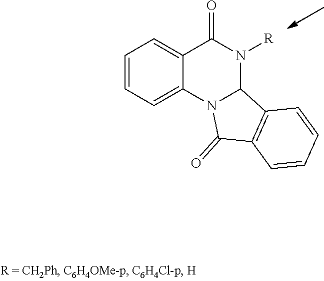

wherein R indicates a non-limiting site of derivatization (Kumar, et al., 2011, Chem. Commun. 47:5010-5012). See FIG. 22 as a non-limiting example.

In certain embodiments, the TNF binder comprise

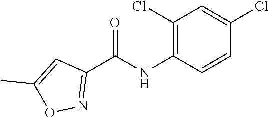 [SMILES: Cc1cc(C(=O)Nc2ccc(Cl)cc2Cl)no1]

### (Jiajiu & Shaw, 2013, Cancer Chemother Pharmacol 72:1-7 (2013).

In certain embodiments, the TNF binder comprises any dihydro-benzo[cd]indole-6-sulfonamide or analogues depicted herein (non-limiting attachment points for REAG include R, or the hydrophobic R group, including naphthyl, on the right hand side of the molecule):

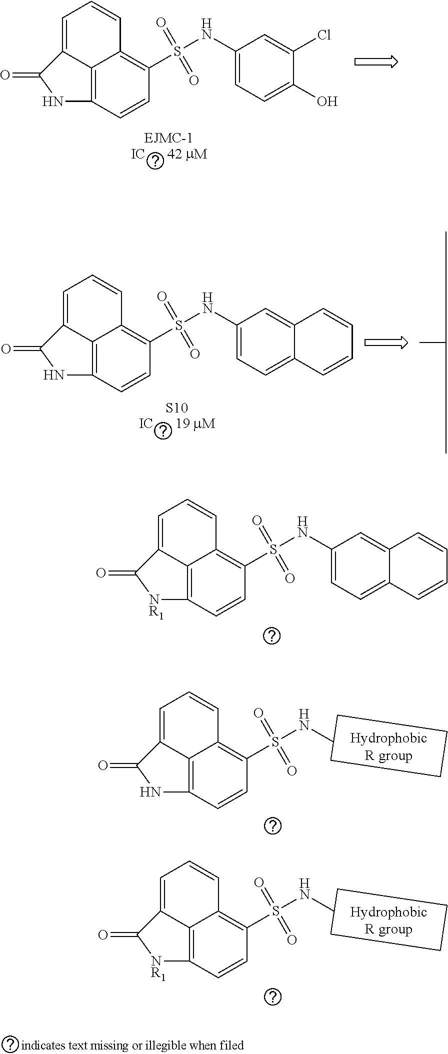

| Com- | |
|---|---|
| pound | Structure |
| EJMC-1 | 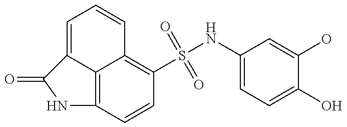 [SMILES: O=C1Nc2ccc(S(=O)(=O)Nc3ccc(O)c(O)c3)c3cccc1c23] |
| S10 | 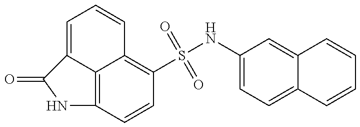 [SMILES: O=C1Nc2ccc(S(=O)(=O)Nc3ccc4ccccc4c3)c3cccc1c23] |
| S21 | 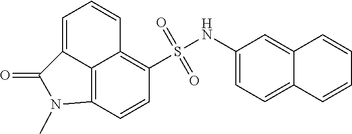 [SMILES: CN1C(=O)c2cccc3c(S(=O)(=O)Nc4ccc5ccccc5c4)ccc1c23] |
| S22 | 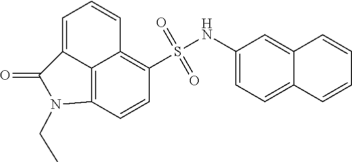 [SMILES: CCN1C(=O)c2cccc3c(S(=O)(=O)Nc4ccc5ccccc5c4)ccc1c23] |
| S23 | 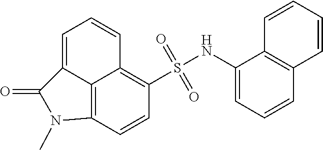 [SMILES: CN1C(=O)c2cccc3c(S(=O)(=O)Nc4cccc5ccccc45)ccc1c23] |
| S24 | 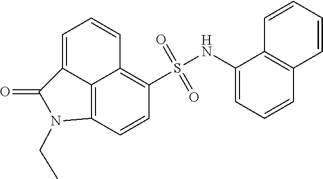 [SMILES: CCN1C(=O)c2cccc3c(S(=O)(=O)Nc4cccc5ccccc45)ccc1c23] |
| S25 | 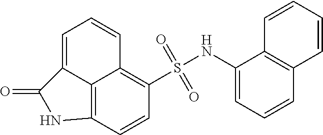 [SMILES: O=C1Nc2ccc(S(=O)(=O)Nc3cccc4ccccc34)c3cccc1c23] |
| S26 | 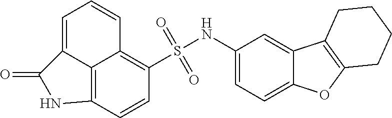 [SMILES: O=C1Nc2ccc(S(=O)(=O)Nc3ccc4oc5c(c4c3)CCCC5)c3cccc1c23] |
| S27 | 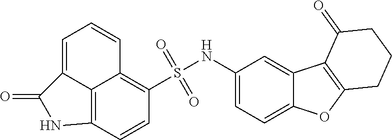 [SMILES: O=C1CCCc2oc3ccc(NS(=O)(=O)c4ccc5c6c(cccc46)C(=O)N5)cc3c21] |
| 4a | 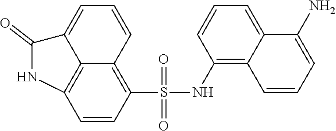 [SMILES: Nc1cccc2c(NS(=O)(=O)c3ccc4c5c(cccc35)C(=O)N4)cccc12] |
| 4c | 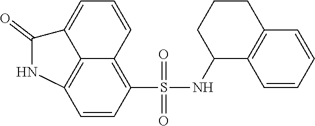 [SMILES: O=C1Nc2ccc(S(=O)(=O)NC3CCCc4ccccc43)c3cccc1c23] |
| 4d | 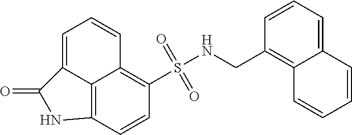 [SMILES: O=C1Nc2ccc(S(=O)(=O)NCc3cccc4ccccc34)c3cccc1c23] |
| 4e | 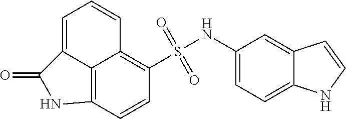 [SMILES: O=C1Nc2ccc(S(=O)(=O)Nc3ccc4[nH]ccc4c3)c3cccc1c23] |
| 4f | 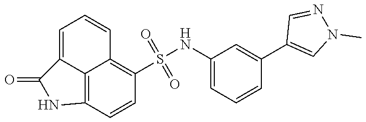 [SMILES: Cn1cc(-c2cccc(NS(=O)(=O)c3ccc4c5c(cccc35)C(=O)N4)c2)cn1] |
| 4g | 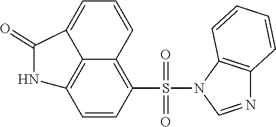 [SMILES: O=C1Nc2ccc(S(=O)(=O)n3cnc4ccccc43)c3cccc1c23] |

In certain embodiments, the TNF binder comprises any of the following:

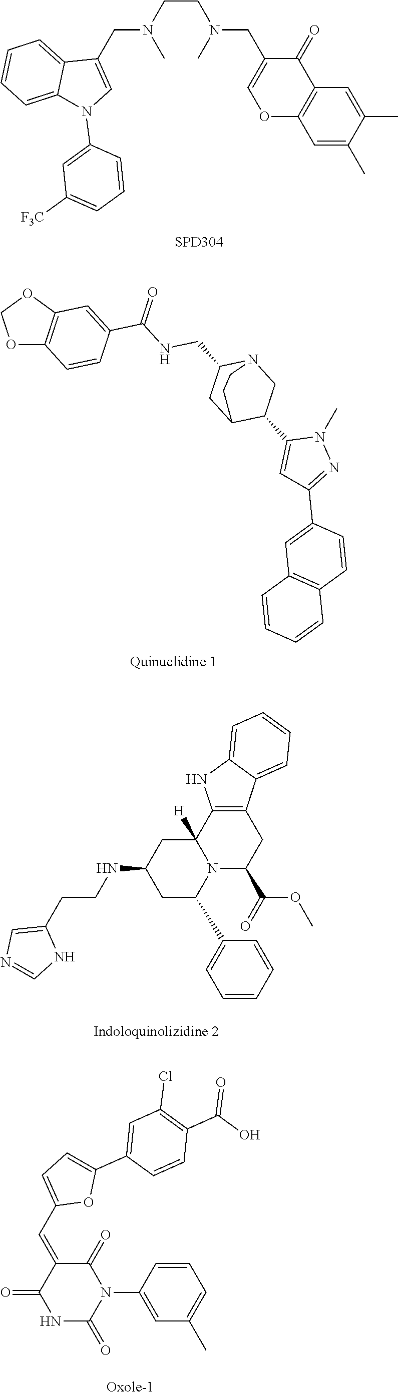 [SMILES: Cc1cc2occ(CN(C)CCN(C)Cc3cn(-c4cccc(C(F)(F)F)c4)c4ccccc34)c(=O)c2cc1C ; "SPD304" ; Cn1nc(-c2ccc3ccccc3c2)cc1[C@H]1CN2CCC1C[C@@H]2CNC(=O)c1ccc2c(c1)OCO2 ; COC(=O)[C@@H]1Cc2c([nH]c3ccccc23)[C@H]2C[C@H](NCCc3cnc[nH]3)C[C@@H](c3ccccc3)N21 ; Cc1cccc(N2C(=O)NC(=O)/C(=C/c3ccc(-c4ccc(C(=O)O)c(Cl)c4)o3)C2=O)c1]

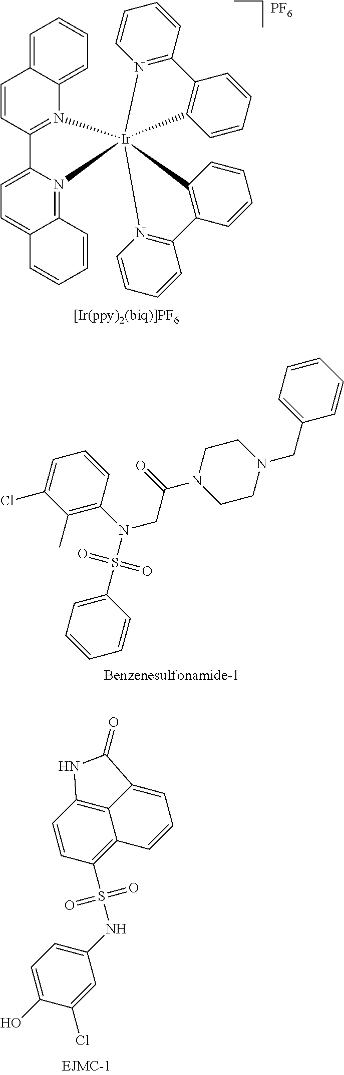

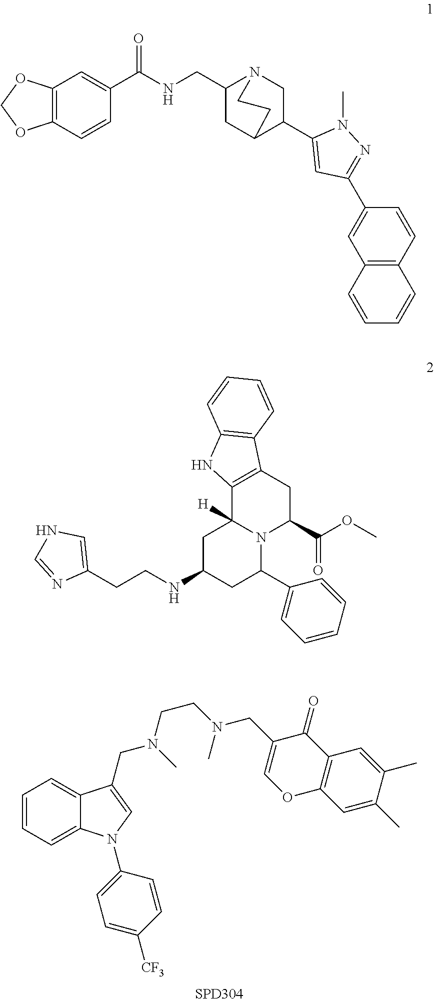 [SMILES: Cn1nc(-c2ccc3ccccc3c2)cc1C1CN2CCC1CC2CNC(=O)c1ccc2c(c1)OCO2 ; COC(=O)[C@@H]1Cc2c([nH]c3ccccc23)[C@H]2C[C@H](NCCc3c[nH]cn3)CC(c3ccccc3)N21 ; Cc1cc2occ(CN(C)CCN(C)Cc3cn(-c4ccc(C(F)(F)F)cc4)c4ccccc34)c(=O)c2cc1C ; "SPD304"]

### (Shiu-Hin Chan, 2010, Angew Chem Int Ed Engl. 49:2860-4).

In certain embodiments, the TNF binder comprises any of the following:

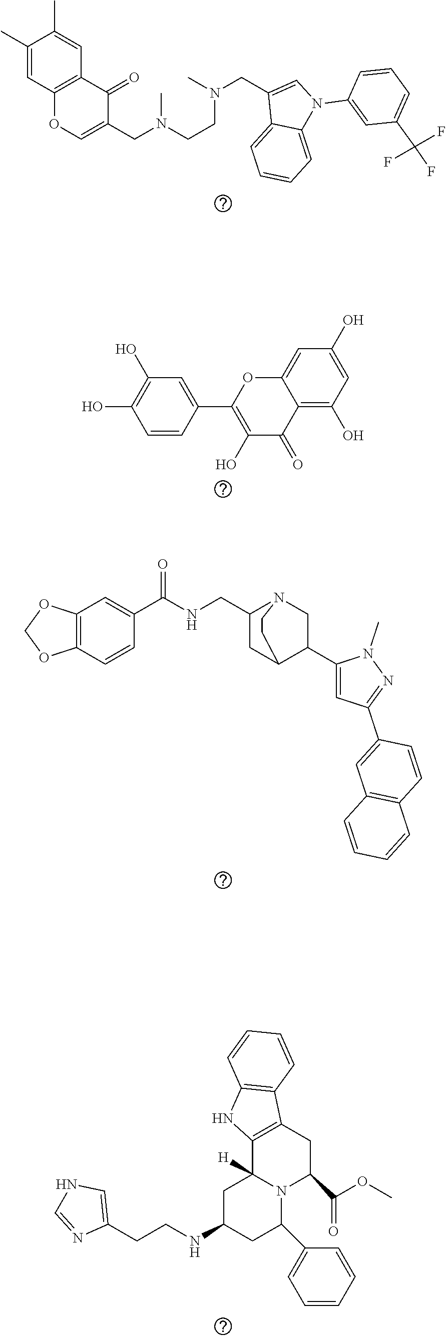 [SMILES: Cc1cc2occ(CN(C)CCN(C)Cc3cn(-c4cccc(C(F)(F)F)c4)c4ccccc34)c(=O)c2cc1C ; O=c1c(O)c(-c2ccc(O)c(O)c2)oc2cc(O)cc(O)c12 ; Cn1nc(-c2ccc3ccccc3c2)cc1C1CN2CCC1CC2CNC(=O)c1ccc2c(c1)OCO2 ; COC(=O)[C@@H]1Cc2c([nH]c3ccccc23)[C@H]2C[C@H](NCCc3c[nH]cn3)CC(c3ccccc3)N21]

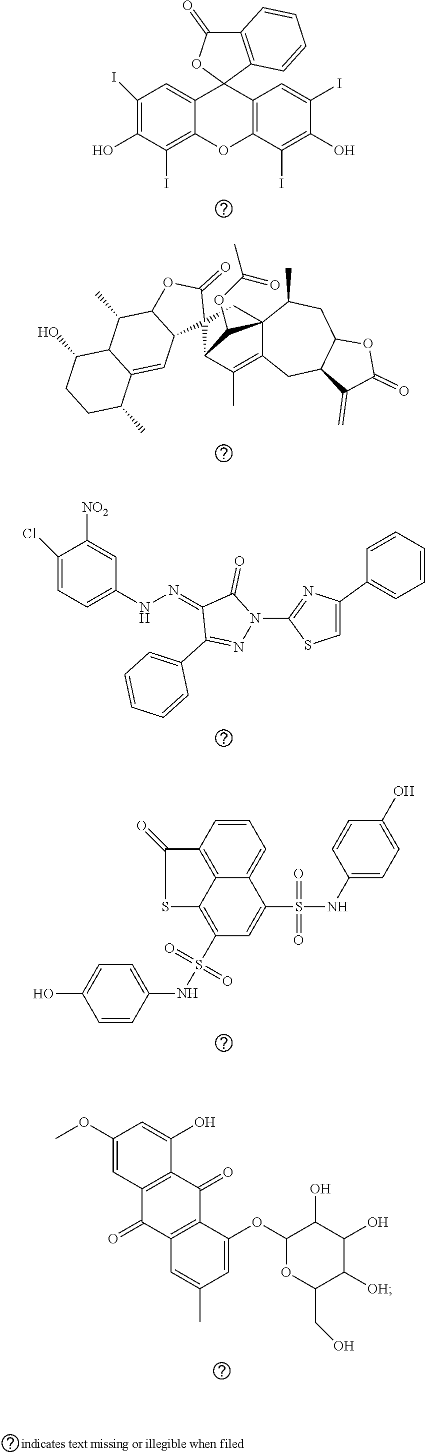

In certain embodiments, the TNF binder comprises any of the following:

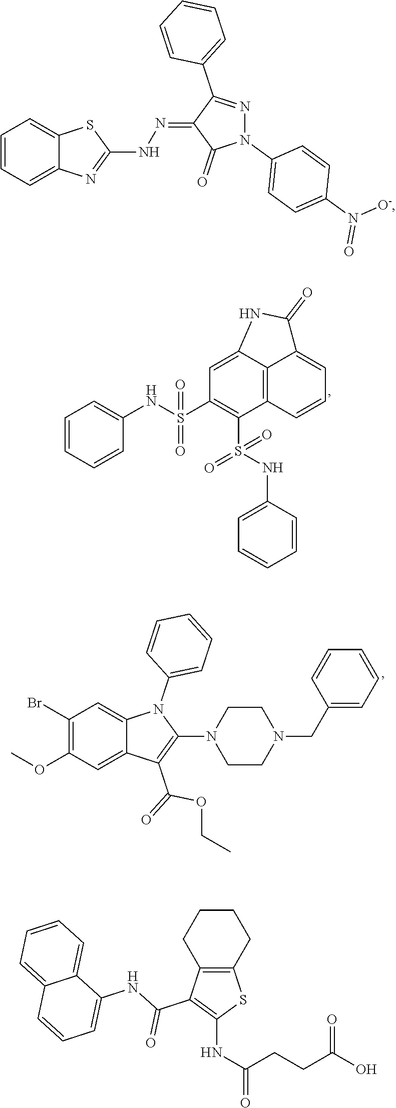

(Chen, et al., 2017, J. Chem. Inf. Model. 57:1101-1111).

In certain embodiments, the TNF binder comprises any compound disclosed in U.S. Pat. No. 10,266,532, which is incorporated herein in its entirety by reference.

In certain embodiments, the TNF binder comprises any compound disclosed in U.S. Pat. No. 9,879,016, which is incorporated herein in its entirety by reference.

In certain embodiments, the TNF binder comprises any compound disclosed in WO 2008/142623, which is incorporated herein in its entirety by reference.

In certain embodiments, the TNF binder comprises

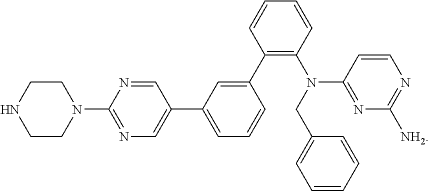 [SMILES: Nc1nccc(N(Cc2ccccc2)c2ccccc2-c2cccc(-c3cnc(N4CCNCC4)nc3)c2)n1]

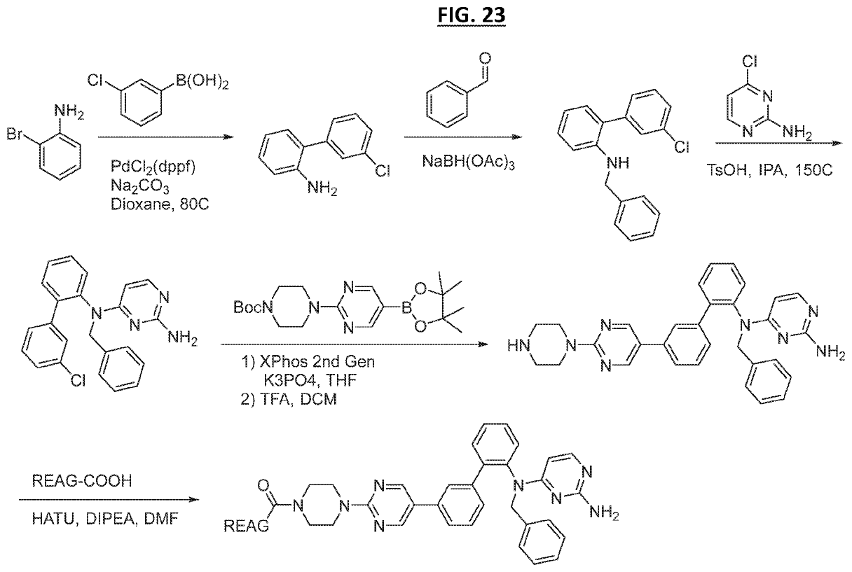

In certain embodiments, the Linker and/or CON can be attached to the compound through the piperizinyl group (Blevitt, et al., 2017, J. Med. Chem. 60:3511-3517). See FIG. 23 as a non-limiting example.

In certain embodiments, the TNF binder comprises a compound of formula (2b):

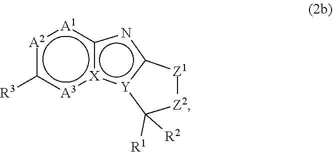

or a pharmaceutically acceptable salt, tautomer, geometric isomer, or stereoisomer thereof, wherein:
R¹ is H, OH, F or optionally substituted (C₁-C₃)alkyl;
R² is optionally substituted aryl, optionally substituted (C₃-C₈)cycloalkyl, optionally substituted heteroaryl or optionally substituted heterocyclyl; or
R¹ and R² together can form an optionally substituted saturated or partially saturated carbocyclic ring or optionally substituted saturated or partially saturated heterocyclic ring;
up to two of A¹, A², and A³ are N, and the rest are independently C(R^{A2});
X is N and Y is C, wherein:
   Z¹ is —C(R^{z})₂— and Z² is —C(R^{z})₂—, —N(R^{z1})— or —O—; or
   Z¹ is —CH₂— and Z² is —Z^{2a}—Z^{2b}—,
      wherein Z^{2a} is attached to Z¹ and Z^{2b} is attached to C(R¹)(R²); and Z^{2a} and
      Z^{2b} are independently —C(R^{z})₂—, —C(R^{z})₂C(R^{z})₂—, —O— or —N(R^{z1})— provided that one of Z^{2a} and Z^{2b} is —C(R^{z})₂— or —C(R^{z})₂C(R^{z})₂—; or —Z^{2a}-Z^{2b}— form —N(R^{z1})C(O)— or —C(O)N(R^{z1})-;
or
X is C and Y is N, provided that R′ is not —OH or —F, wherein:
   Z¹ is —C(R^{z})₂— and Z² is —C(R^{z})₂—; or
   Z¹ is —C(R^{z})₂— and Z² is —Z^{2a}—Z^{2b}—,
      wherein Z^{2a} is attached to Z¹ and Z^{2b} is attached to C(R¹)(R²), and Z^{2a} is —C(R^{z})₂—, —C(R^{z})₂C(R^{z})₂—, —O— or —N(R^{z1}), and Z^{2b} is —C(R)₂-, or —Z^{2a}—Z^{2b}— form —N(R^{z1})C(O)— or —C(O)N(R^{z1})—;
R³ is —R^{3a}—R^{3b}, wherein:
   R^{3a} is an optionally substituted aryl, optionally substituted saturated or partially saturated heterocyclyl or optionally substituted heteroaryl;
   R^{3b} is H, —CF₃, —CN, —C(O)OH, —N(R^{a})(R^{b}), —C(O)N(R^{a})(R^{b}), —C(O)— optionally substituted heterocyclyl, —O(R^{a}), —S(O)₂(C₁-C₃)alkyl, —S(O)₂N(R^{c})(R^{d}), —S—(C₁-C₃)alkyl, —S(O)₂-R optionally substituted (C₁-C₅)alkyl, —(CH₂)ₚ-optionally substituted (C₃-C₆)cycloalkyl, —(CH₂)ₚ-optionally substituted heteroaryl or —(CH₂)ₚ-optionally substituted saturated, unsaturated or partially saturated heterocyclyl; provided that R^{3b} is not H or methoxy when R² is optionally substituted phenyl;
   R^{a} and R^{b} are independently selected from H, optionally substituted (C₁-C₅)alkyl, —C(O)— optionally substituted (C₁-C₅)alkyl, optionally substituted —(CH₂)ₚ-(C₃-C₆)cycloalkyl and —(CH₂)ₚ-optionally substituted heterocyclyl;
R^{c} and R^{d} are independently selected from H, optionally substituted (C₁-C₅)alkyl, optionally substituted —(CH₂)ₚ C₃-C₆)cycloalkyl and —(CH₂)ₚ-optionally substituted heterocyclyl;
R^{A2} is independently H, CF₃, halo or (C₁-C₃)alkyl;
R^{z} is independently H, F, CF₃, —OH or (C₁-C₃)alkyl;
R^{z1} is independently H or (C₁-C₃)alkyl; and
p is independently 0, 1 or 2.

In certain embodiments, R² is not phenyl substituted with —OCHF₂.

In certain embodiments, the compound is not 1-(2-methylphenyl)-7-[2-(morpholin-4-yl)pyrimidin-5-yl]-2,3-dihydro-1H-pyrrolo[1,2-a]benzimidazole; 7-[2-(morpholin-4-yl)pyrimidin-5-yl]-1-phenyl-2,3-dihydro-1H-pyrrolo[1,2-a]benzimidazole; (1R or S)-7-(6-methylsulfonyl-3-pyridyl)-1-phenyl-2,3-dihydro-1H-pyrrolo[1,2-a]benzimidazole; [5-[(1R or S)-1-phenyl-2,3-dihydro-1H-pyrrolo[1,2-a]benzimidazol-7-yl]-2-pyridyl]methanol; tert-butyl 4-[5-[(1R or S)-1-phenyl-2,3-dihydro-1H-pyrrolo[1,2-a]benzimidazol-7-yl]-2-pyridyl]piperazine-1-carboxylate; (1R or S)-7-[6-chloromethyl)-3-pyridyl]-1-phenyl-2,3-dihydro-1H-pyrrolo[1,2-a]benzimidazole; (1R or S)-7-[(6-(methylsulfonylmethyl)-3-pyridyl]-1-phenyl-2,3-dihydro-1H-pyrrolo[1,2-a]benzimidazole; or (1R or S)-1-phenyl-7-[6-(piperazin-1-yl)pyridine-3-yl]-2,3-dihydro-1H-pyrrolo[1,2-a]benzimidazole.

In certain embodiments, the compound is 1-(2-methylphenyl)-7-[2-(morpholin-4-yl)pyrimidin-5-yl]-2,3-dihydro-1H-pyrrolo[1,2-a]benzimidazole; 7-[2-(morpholin-4-yl)pyrimidin-5-yl]-1-phenyl-2,3-dihydro-1H-pyrrolo[1,2-a]benzimidazole; (1R or S)-7-(6-methylsulfonyl-3-pyridyl)-1-phenyl-2,3-dihydro-1H-pyrrolo[1,2-a]benzimidazole; [5-[(1R or S)-1-phenyl-2,3-dihydro-1H-pyrrolo[1,2-a]benzimidazol-7-yl]-2-pyridyl]methanol; tert-butyl 4-[5-[(1R or S)-1-phenyl-2,3-dihydro-1H-pyrrolo[1,2-a]benzimidazol-7-yl]-2-pyridyl]piperazine-1-carboxylate; (1R or S)-7-[6-chloromethyl)-3-pyridyl]-1-phenyl-2,3-dihydro-1H-pyrrolo[1,2-a]benzimidazole; (1R or S)-7-[(6-(methylsulfonylmethyl)-3-pyridyl]-1-phenyl-2,3-dihydro-1H-pyrrolo[1,2-a]benzimidazole; or (1R or S)-1-phenyl-7-[6-(piperazin-1-yl)pyridine-3-yl]-2,3-dihydro-1H-pyrrolo[1,2-a]benzimidazole.

In certain embodiments, the compound of formula (2a) comprises one of the following:

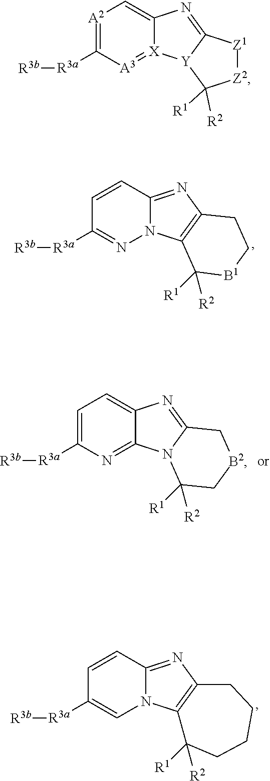

wherein A² is CH or N; A³ is CH or N; B¹ is CH₂ or O; B² is CH₂ or; X is C or N; Y is C or N;
Z¹ is CH or O; and Z² is CH₂ or O.

In certain embodiments, R^{3a} is selected from the group consisting of:

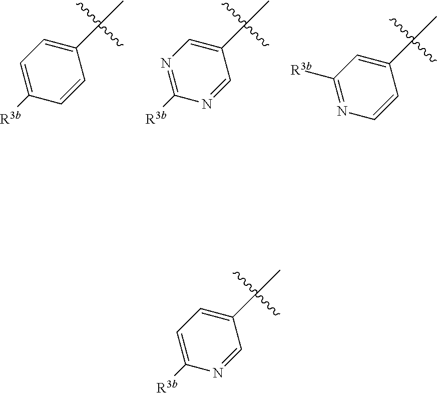

In certain embodiments, R^{3b} is selected from the group consisting of:

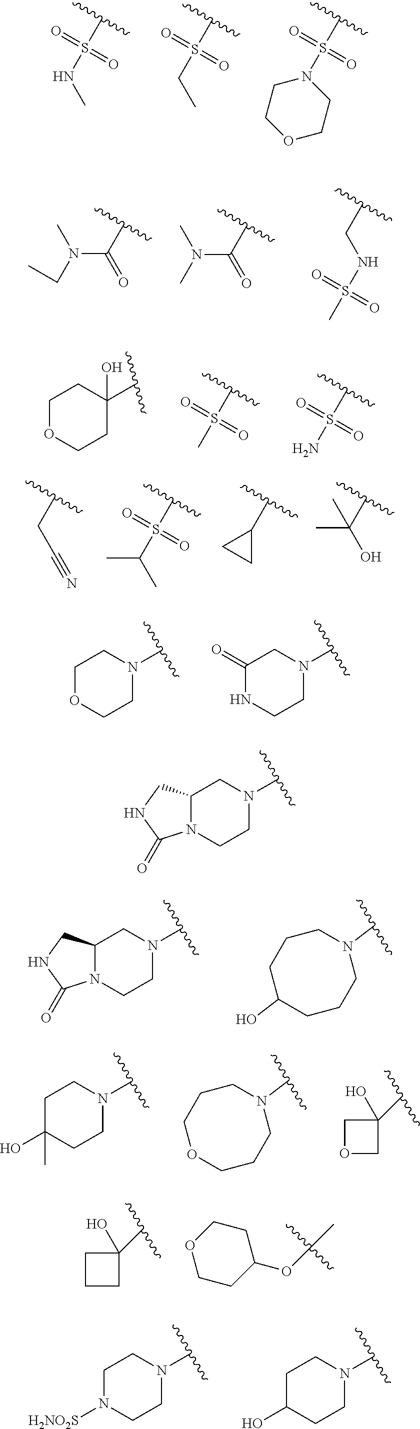

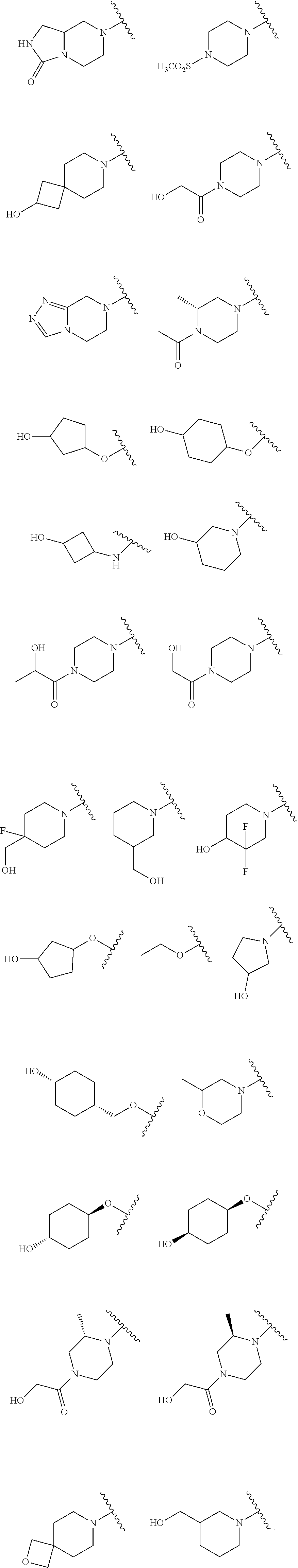

In certain embodiments, R^{3a} or R^{3b} can be used to attach the TNF linker to the compound of the disclosure. This can be done, for example, using any hydroxyl, amino, amido, thiyl, or carboxylic acid group that is present in R^{3a} or R^{3b} as listed herein or that can be introduced therein. In any such cases, the hydroxyl group in R^{3a} or R^{3b} can be used for example to form an ester bond; the carboxylic group in R^{3a} or R^{3b} can be used for example to form an ester bond or an amide bond; the amino group in R^{3a} or R^{3b} can be used for example to form an amide group and an imine group, and so forth; the amino, amido, or thiyl group in R^{3a} or R^{3b} can be used for example to form a chemical linkage through alkylation or nucleophilic displacement, and so forth, as known to those skilled in the art.

In certain embodiments, R¹ is selected from the group consisting of H, methyl, and hydroxyl.

In certain embodiments, R¹ and R² combine to form one of the following:

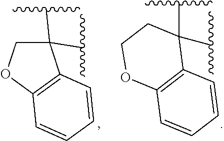

In certain embodiments, R⁴ is selected from the group consisting of:

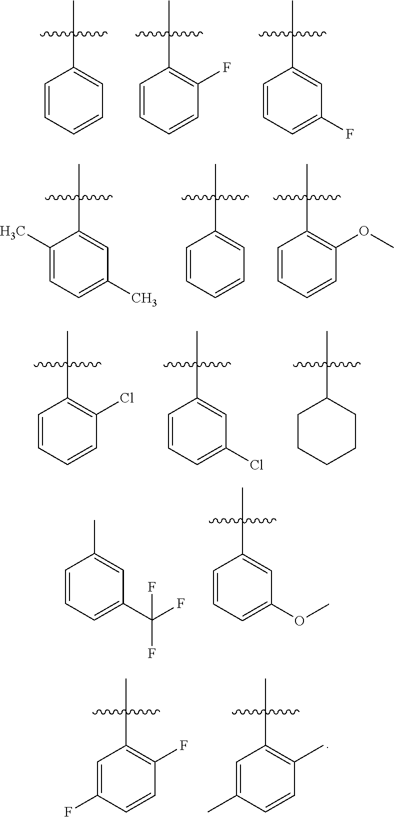

In certain embodiments, the compound is selected from the group consisting of:
2-(5-(1-(2-methoxyphenyl)-2,3-dihydro-1H-benzo[d]pyrrolo[1,2-a]imidazol-7-yl)pyrimidin-2-yl)propan-2-ol;
4-(3-fluorophenyl)-7-(2-morpholinopyrimidin-5-yl)-3,4-dihydro-1H-benzo[4,5]imidazo[2,1-c][1,4]oxazine;
(R)-1-phenyl-7-(2-((tetrahydro-2H-pyran-4-yl)oxy)pyridin-4-yl)-2,3-dihydro-1H-benzo[d]pyrrolo[1,2-a]imidazole;
(S)-2-(2-morpholinopyrimidin-5-yl)-9-phenyl-8,9-dihydro-6H-pyrido[3′,2′:4,5]imidazo[2,1-c][1,4]oxazine;
2-(5-(8-methyl-8-phenyl-7,8-dihydro-6H-pyrrolo[2′,1′:2,3]imidazo[4,5-b]pyridin-2-yl)pyrimidin-2-yl)propan-2-ol;
2-(5-(1-(tetrahydro-2H-pyran-4-yl)-2,3-dihydro-1H-benzo[d]pyrrolo[1,2-a]imidazol-7-yl)pyrimidin-2-yl)propan-2-ol;
(S)-7-(2-((1R,6S)-3,10-diazabicyclo[4.3.1]decan-10-yl)pyrimidin-5-yl)-4-phenyl-3,4-dihydro-1H-benzo[4,5]imidazo[2,1-c][1,4]oxazine;
(S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-amine;
7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-(8-(2-methoxyphenyl)-7,8-dihydro-6H-cyclopenta[4,5]imidazo[1,2-b]pyridazin-2-yl)pyrimidin-2-yl)hexahydroimidazo[1,5-a]pyrazine-3(2H)-one;
1-(5-(8-phenyl-7,8-dihydro-6H-cyclopenta[4,5]imidazo[1,2-b]pyridazin-2-yl)pyrimidin-2-yl)piperidin-4-ol;
2-(5-(4-(2-methoxyphenyl)-3,4-dihydro-1H-pyran[3′,4′:4,5]imidazo[1,2-a]pyridin-7-yl)pyrimidin-2-yl)propan-2-ol;
(S)-7-(5-((R)-8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
(R)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one;
2-(5-(8-(pyridin-2-yl)-7,8-dihydro-6H-pyrrolo[2′,1′:2,3]imidazo[4,5-b]pyridin-2-yl)pyrimidin-2-yl)propan-2-ol;
2-(5-(1-(pyridin-2-yl)-2,3-dihydro-1H-benzo[d]pyrrolo[1,2-a]imidazol-7-yl)pyrimidin-2-yl)propan-2-ol;
(S)-7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
(R)-7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-(9-(2-methoxyphenyl)-6,7,8,9-tetrahydrobenzo[4,5]imidazo[1,2-a]pyridin-2-yl)pyrimidin-2-yl)-7-azaspiro[3.5]nonan-2-ol;
4-(5-(9-(2-methoxyphenyl)-6,7,8,9-tetrahydrobenzo[4,5]imidazo[1,2-a]pyridin-2-yl)pyrimidin-2-yl)-1,4-oxazepane;
7-(5-(9-(2-methoxyphenyl)-6,7,8,9-tetrahydrobenzo[4,5]imidazo[1,2-a]pyridin-2-yl)pyrimidin-2-yl)hexahydroimidazo[1,5-a]pyrazin-3(2H)-one;
1-(5-(9-(2-methoxyphenyl)-6,7,8,9-tetrahydrobenzo[4,5]imidazo[1,2-a]pyridin-2-yl)pyrimidin-2-yl)-4-methylpiperidin-4-ol;
(4-fluoro-1-(5-(9-(2-methoxyphenyl)-6,7,8,9-tetrahydrobenzo[4,5]imidazo[1,2-a]pyridin-2-yl)pyrimidin-2-yl)piperidin-4-yl)methanol;
(4-fluoro-1-(5-(9-(2-methoxyphenyl)-6,7,8,9-tetrahydrobenzo[4,5]imidazo[1,2-a]pyridin-2-yl)pyrimidin-2-yl)piperidin-4-yl)methanol;
1-(5-(9-(2-methoxyphenyl)-6,7,8,9-tetrahydrobenzo[4,5]imidazo[1,2-a]pyridin-2-yl)pyrimidin-2-yl)azepan-4-ol;
1-(5-(9-(2-methoxyphenyl)-6,7,8,9-tetrahydrobenzo[4,5]imidazo[1,2-a]pyridin-2-yl)pyrimidin-2-yl)piperidin-4-ol;
1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol;
1-(5-(9-(3-fluorophenyl)-8,9-dihydro-6H-pyrido[3′,2′:4,5]imidazo[2,1-c][1,4]oxazin-2-yl)pyrimidin-2-yl)piperidin-4-ol;
7-(5-(8-methyl-8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
7-(5-(9-(3-fluorophenyl)-8,9-dihydro-6H-pyrido[3′,2′:4,5]imidazo[2,1-c][1,4]oxazin-2-yl)pyrimidin-2-yl)hexahydroimidazo[1,5-a]pyrazin-3(2H)-one;
7-(5-(8-cyclohexyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
7-(5-((R)-8-cyclohexyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
7-(5-((S)-4-(2-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-(4-(3-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-(4-(2-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one;
4-(5-(1-(2-methoxyphenyl)-2,3-dihydro-1H-cyclopenta[4,5]imidazo[1,2-a]pyridin-7-yl)pyrimidin-2-yl)morpholine;
(R)-7-(5-((R)-9-phenyl-8,9-dihydro-6H-pyrano[3′,4′:4,5]imidazo[1,2-b]pyridazin-2-yl)pyrimidin-2-yl)hexahydroimidazo[1,5-a]pyrazin-3(2H)-one;
(R)-7-(5-((R)-4-phenyl-3,4-dihydro-1H-pyrano[3′,4′:4,5]imidazo[1,2-a]pyridin-7-yl)pyrimidin-2-yl)hexahydroimidazo[1,5-a]pyrazin-3(2H)-one;
1-(5-(9-(2-methoxyphenyl)-6,7,8,9-tetrahydroimidazo[1,2-a:5,4-b′]dipyridin-2-yl)pyrimidin-2-yl)piperidin-4-ol;
7-(5-(8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
1-(5-(8-(2-methoxyphenyl)-7,8-dihydro-6H-pyrrolo[2′,1′:2,3]imidazo[4,5-b]pyridin-2-yl)pyrimidin-2-yl)piperidin-4-ol;
(S)-1-(5-(9-(2-methoxyphenyl)-8,9-dihydro-6H-pyrido[3′,2′:4,5]imidazo[2,1-c][1,4]oxazin-2-yl)pyrimidin-2-yl)piperidin-4-ol;
7-(5-((S)-9-phenyl-8,9-dihydro-6H-pyrido[3′,2′:4,5]imidazo[2,1-c][1,4]oxazin-2-yl)pyrimidin-2-yl)hexahydroimidazo[1,5-a]pyrazin-1(5H)-one;
(S)-1-(5-(9-phenyl-8,9-dihydro-6H-pyrido[3′,2′:4,5]imidazo[2,1-c][1,4]oxazin-2-yl)pyrimidin-2-yl)piperidin-4-ol;
(S)-4-(5-(9-phenyl-8,9-dihydro-6H-pyrido[3′,2′:4,5]imidazo[2,1-c][1,4]oxazin-2-yl)pyrimidin-2-yl)piperazin-2-one;
(S)-2-(5-(9-phenyl-8,9-dihydro-6H-pyrido[3′,2′:4,5]imidazo[2,1-c][1,4]oxazin-2-yl)pyrimidin-2-yl)propan-2-ol;
(S)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one;
(R)-3-(5-(1-phenyl-2,3-dihydro-1H-benzo[d]pyrrolo[1,2-a]imidazol-7-yl)pyrimidin-2-yl)oxetan-3-ol;
(R)-1-(5-41-phenyl-2,3-dihydro-1H-benzo[d]pyrrolo[1,2-a]imidazol-7-yl)pyrimidin-2-yl)cyclobutanol;
(R)-4-(5-(1-phenyl-2,3-dihydro-1H-benzo[d]pyrrolo[1,2-a]imidazol-7-yl)pyrimidin-2-yl)tetrahydro-2H-pyran-4-ol;
(R)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one;
2-(5-(4-(2,6-dichlorophenyl)-3,4-dihydro-1H-benzo[4,5]imidazo[2,1-c][1,4]oxazin-7-yl)pyrimidin-2-yl)propan-2-ol;
(S)-2-(5-(4-(2-methoxyphenyl)-3,4-dihydro-1H-benzo[4,5]imidazo[2,1-c][1,4]oxazin-7-yl)pyrimidin-2-yl)propan-2-ol;
7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-(4-(2-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-(1,2′,3,3′-tetrahydrospiro[benzo[4,5]imidazo[2,1-c][1,4]oxazine-4,1′-inden]-7-yl)pyrimidin-2-yl)hexahydroimidazo[1,5-a]pyrazin-3(2H)-one;
(S)-2-hydroxy-1-(4-(5-(4-phenyl-3,4-dihydro-1H-benzo[4,5]imidazo[2,1-c][1,4]oxazin-7-yl)pyrimidin-2-yl)piperazin-1-yl)ethanone;
7-(5-((S)-4-phenyl-3,4-dihydro-1H-benzo[4,5]imidazo[2,1-c][1,4]oxazin-7-yl)pyrimidin-2-yl)-7-azaspiro[3.5]nonan-1-ol;
(R)-1-(5-(8-(3-fluorophenyl)-7,8-dihydro-6H-pyrrolo[2′,1′:2,3]imidazo[4,5-b]pyridin-2-yl)pyrimidin-2-yl)piperidin-4-ol;
(R)-1-(5-(8-phenyl-7,8-dihydro-6H-pyrrolo[2′,1′:2,3]imidazo[4,5-b]pyridin-2-yl)pyrimidin-2-yl)piperidin-4-ol;
(R)-4-(5-(8-phenyl-7,8-dihydro-6H-pyrrolo[2′,1′:2,3]imidazo[4,5-b]pyridin-2-yl)pyrimidin-2-yl)morpholine;
(S)-2-(5-(1-(2,5-dimethylphenyl)-2,3-dihydro-1H-benzo[d]pyrrolo[1,2-a]imidazol-7-yl)pyrimidin-2-yl)propan-2-ol;
(R)-2-(5-(1-(2,5-dimethylphenyl)-2,3-dihydro-1H-benzo[d]pyrrolo[1,2-a]imidazol-7-yl)pyrimidin-2-yl)propan-2-ol;
(S)-4-(3-fluorophenyl)-7-(2-morpholinopyrimidin-5-yl)-3,4-dihydro-1H-benzo[4,5]imidazo[2,1-c][1,4]oxazine;
7-(5-(8-(2,5-dimethylphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one compound with ethane (1:1);
7-(5-(8-(3-fluorophenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
(R)-1-(5-(1-phenyl-2,3-dihydro-1H-benzo[d]pyrrolo[1,2-a]imidazol-7-yl)pyrimidin-2-yl)piperidin-4-ol;
(R)-4-(5-(1-phenyl-2,3-dihydro-1H-benzo[d]pyrrolo[1,2-a]imidazol-7-yl)pyrimidin-2-yl)thiomorpholine 1,1-dioxide;
-(5-(8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one;
3,3-difluoro-1-(5-((R)-1-phenyl-2,3-dihydro-1H-benzo[d]pyrrolo[1,2-a]imidazol-7-yl)pyrimidin-2-yl)piperidin-4-ol;
(S)-2-(5-(4-(2-(difluoromethoxy)phenyl)-3,4-dihydro-1H-benzo[4,5]imidazo[2,1-c][1,4]oxazin-7-yl)pyrimidin-2-yl)propan-2-ol;
(S)-2-(5-(9-(2-methoxyphenyl)-6,7,8,9-tetrahydrobenzo[4,5]imidazo[1,2-a]pyridin-2-yl)pyrimidin-2-yl)propan-2-ol;
(R)-7-(5-(1-phenyl-2,3-dihydro-1H-benzo[d]pyrrolo[1,2-a]imidazol-7-yl)pyrimidin-2-yl)-7-azaspiro[3.5]nonan-2-ol;
1-(5-(8-(2,5-dimethylphenyl)-7,8-dihydro-6H-pyrrolo[2′,1′:2,3]imidazo[4,5-b]pyridin-2-yl)pyrimidin-2-yl)piperidin-4-ol;
4-(5-(10-(2-methoxyphenyl)-7,8,9,10-tetrahydro-6H-cyclohepta[4,5]imidazo[1,2-a]pyridin-2-yl)pyrimidin-2-yl)morpholine;
(R)-4-(5-(1-phenyl-2,3-dihydro-1H-benzo[d]pyrrolo[1,2-a]imidazol-7-yl)pyrimidin-2-yl)morpholine;
7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
4-(5-(8-(2,5-dimethylphenyl)-7,8-dihydro-6H-pyrrolo[2′,1′:2,3]imidazo[4,5-b]pyridin-2-yl)pyrimidin-2-yl)piperazin-2-one;
2-(5-(1-(2,5-dimethylphenyl)-2,3-dihydro-1H-benzo[d]pyrrolo[1,2-a]imidazol-7-yl)pyrimidin-2-yl)propan-2-ol;
3,3-difluoro-1-(5-((S)-4-phenyl-3,4-dihydro-1H-benzo[4,5]imidazo[2,1-c][1,4]oxazin-7-yl)pyrimidin-2-yl)piperidin-4-ol;
7-(5-(4-(3-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one;
(R)-2-(5-(1-(2-methoxyphenyl)-2,3-dihydro-1H-cyclopenta[4,5]imidazo[1,2-a]pyridin-7-yl)pyrimidin-2-yl)propan-2-ol;
7-(4-(isopropylsulfonyl)phenyl)-1-phenyl-2,3-dihydro-1H-benzo[d]pyrrolo[1,2-a]imidazole;
2-(5-(9-(2-methoxyphenyl)-6,7,8,9-tetrahydrobenzo[4,5]imidazo[1,2-a]pyridin-2-yl)pyrimidin-2-yl)propan-2-ol;
4-(5-(8-(2,5-dimethylphenyl)-7,8-dihydro-6H-pyrrolo[2′,1′:2,3]imidazo[4,5-b]pyridin-2-yl)pyrimidin-2-yl)morpholine;
(S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol;
4-(5-(9-(2-methoxyphenyl)-6,7,8,9-tetrahydrobenzo[4,5]imidazo[1,2-a]pyridin-2-yl)pyrimidin-2-yl)morpholine;
(S)-1-(5-(4-phenyl-3,4-dihydro-1H-benzo[4,5]imidazo[2,1-c][1,4]oxazin-7-yl)pyrimidin-2-yl)piperidin-4-ol;
1-(5-(9-phenyl-6,7,8,9-tetrahydroimidazo[1,2-a:5,4-b′]dipyridin-2-yl)pyrimidin-2-yl)piperidin-4-ol;
(R)-2-(5-(1-phenyl-2,3-dihydro-1H-benzo[d]pyrrolo[1,2-a]imidazol-7-yl)pyrimidin-2-yl)propan-2-ol;
4-(5-(8-(3-fluorophenyl)-7,8-dihydro-6H-pyrrolo[2′,1′:2,3]imidazo[4,5-b]pyridin-2-yl)pyrimidin-2-yl)morpholine;
2-(5-(10-(2-methoxyphenyl)-7,8,9,10-tetrahydro-6H-cyclohepta[4,5]imidazo[1,2-a]pyridin-2-yl)pyrimidin-2-yl)propan-2-ol;
N-methyl-4-(1-phenyl-2,3-dihydro-1H-benzo[d]pyrrolo[1,2-a]imidazol-7-yl)benzenesulfonamide;
1-(5-(8-(3-fluorophenyl)-7,8-dihydro-6H-pyrrolo[2′,1′:2,3]imidazo[4,5-b]pyridin-2-yl)pyrimidin-2-yl)piperidin-4-ol;
7-(4-(ethylsulfonyl)phenyl)-1-phenyl-2,3-dihydro-1H-benzo[d]pyrrolo[1,2-a]imidazole;
(S)-7-(2-morpholinopyrimidin-5-yl)-4-phenyl-3,4-dihydro-1H-benzo[4,5]imidazo[2,1-c][1,4]oxazine;
4-(5-(8-phenyl-7,8-dihydro-6H-pyrrolo[2′,1′:2,3]imidazo[4,5-b]pyridin-2-yl)pyrimidin-2-yl)morpholine;
4-(5-(8-phenyl-7,8-dihydro-6H-pyrrolo[2′,1′:2,3]imidazo[4,5-b]pyridin-2-yl)pyrimidin-2-yl)morpholine;
(S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol;
1-(5-(8-phenyl-7,8-dihydro-6H-pyrrolo[2′,1′:2,3]imidazo[4,5-b]pyridin-2-yl)pyrimidin-2-yl)piperidin-4-ol;
(S)-7-(2-(1,4-oxazepan-4-yl)pyrimidin-5-yl)-4-phenyl-3,4-dihydro-1H-benzo[4,5]imidazo[2,1-c][1,4]oxazine;
4-(5-(1-phenyl-2,3-dihydro-1H-benzo[d]pyrrolo[1,2-a]imidazol-7-yl)pyrimidin-2-yl)morpholine;
3,3-difluoro-1-(5-(4-(3-fluorophenyl)-3,4-dihydro-1H-benzo[4,5]imidazo[2,1-c][1,4]oxazin-7-yl)pyrimidin-2-yl)piperidin-4-ol;
(1-(5-((S)-4-phenyl-3,4-dihydro-1H-benzo[4,5]imidazo[2,1-c][1,4]oxazin-7-yl)pyrimidin-2-yl)piperidin-3-yl)methanol;
1-(5-((S)-4-phenyl-3,4-dihydro-1H-benzo[4,5]imidazo[2,1-c][1,4]oxazin-7-yl)pyrimidin-2-yl)azepan-4-ol;
(S)-4-(5-(4-phenyl-3,4-dihydro-1H-benzo[4,5]imidazo[2,1-c][1,4]oxazin-7-yl)pyrimidin-2-yl)piperazine-1-sulfonamide;
N-(4-(1-phenyl-2,3-dihydro-1H-benzo[d]pyrrolo[1,2-a]imidazol-7-yl)benzyl)methanesulfonamide;
2-(5-(1-(2-methoxyphenyl)-2,3-dihydro-1H-cyclopenta[4,5]imidazo[1,2-a]pyridin-7-yl)pyrimidin-2-yl)propan-2-ol;
7-(4-(methylsulfonyl)phenyl)-1-phenyl-2,3-dihydro-1H-benzo[d]pyrrolo[1,2-a]imidazole;
7-(2-(1,4-oxazepan-4-yl)pyrimidin-5-yl)-4-(3-fluorophenyl)-3,4-dihydro-1H-benzo[4,5]imidazo[2,1-c][1,4]oxazine;
4-(1-phenyl-2,3-dihydro-1H-benzo[d]pyrrolo[1,2-a]imidazol-7-yl)benzenesulfonamide;
(4-(5-((S)-4-phenyl-3,4-dihydro-1H-benzo[4,5]imidazo[2,1-c][1,4]oxazin-7-yl)pyrimidin-2-yl)morpholin-2-yl)methanol;
(S)-4-(5-(4-phenyl-3,4-dihydro-1H-benzo[4,5]imidazo[2,1-c][1,4]oxazin-7-yl)pyrimidin-2-yl)thiomorpholine 1,1-dioxide;
4-(5-(9-phenyl-6,7,8,9-tetrahydroimidazo[1,2-a:5,4-b′]dipyridin-2-yl)pyrimidin-2-yl)morpholine;
(R)-4-(5-(1-phenyl-1,2,3,4-tetrahydrobenzo[4,5]imidazo[1,2-a]pyridin-8-yl)pyrimidin-2-yl)morpholine;
2-(5-(1-(3-fluorophenyl)-2,3-dihydro-1H-benzo[d]pyrrolo[1,2-a]imidazol-7-yl)pyrimidin-2-yl)propan-2-ol;
(S)-(4-fluoro-1-(5-(4-phenyl-3,4-dihydro-1H-benzo[4,5]imidazo[2,1-c][1,4]oxazin-7-yl)pyrimidin-2-yl)piperidin-4-yl)methanol;
4-(5-(9-(3-chlorophenyl)-6,7,8,9-tetrahydrobenzo[4,5]imidazo[1,2-a]pyridin-2-yl)pyrimidin-2-yl)morpholine;
(R)-2-(5-(1-phenyl-1,2,3,4-tetrahydrobenzo[4,5]imidazo[1,2-a]pyridin-8-yl)pyrimidin-2-yl)propan-2-ol;
(S)-7-(2-(1′-methyl-[4,4′-bipiperidin]-1-yl)pyrimidin-5-yl)-4-phenyl-3,4-dihydro-1H-benzo[4,5]imidazo[2,1-c][1,4]oxazine;
2-(5-(9-phenyl-6,7,8,9-tetrahydroimidazo[1,2-a:5,4-b′]dipyridin-2-yl)pyrimidin-2-yl)propan-2-ol;
7-(2-Morpholinopyrimidin-5-yl)-4-phenyl-3,4-dihydro-1H-benzo[4,5]imidazo[2,1-c][1,4]oxazine;
(4S)-7-(2-(2-methylmorpholino)pyrimidin-5-yl)-4-phenyl-3,4-dihydro-1H-benzo[4,5]imidazo[2,1-c][1,4]oxazine;
7-(5-(4-(3-fluorophenyl)-3,4-dihydro-1H-benzo[4,5]imidazo[2,1-c][1,4]oxazin-7-yl)pyrimidin-2-yl)-7-azaspiro[3.5]nonan-2-ol;
4-(5-(1-phenyl-1,2,3,4-tetrahydrobenzo[4,5]imidazo[1,2-a]pyridin-8-yl)pyrimidin-2-yl)morpholine;
Ethyl 2-[[5-[9-(2-methoxyphenyl)-6,7,8,9-tetrahydropyrido[1,2-a]benzimidazol-2-yl]pyrimidin-2-yl]amino]acetate;
(S)-7-(2-(5,6-dihydro-[1,2,4]triazolo[4,3-a]pyrazin-7(8H)-yl)pyrimidin-5-yl)-4-phenyl-3,4-dihydro-1H-benzo[4,5]imidazo[2,1-c][1,4]oxazine;
2-(5-(8-(3-fluorophenyl)-7,8-dihydro-6H-pyrrolo[2′,1′:2,3]imidazo[4,5-b]pyridin-2-yl)pyrimidin-2-yl)propan-2-ol;
(S)-7-(2-(4-(methylsulfonyl)piperazin-1-yl)pyrimidin-5-yl)-4-phenyl-3,4-dihydro-1H-benzo[4,5]imidazo[2,1-c][1,4]oxazine;
2-(5-(8-phenyl-7,8-dihydro-6H-pyrrolo[2′,1′:2,3]imidazo[4,5-b]pyridin-2-yl)pyrimidin-2-yl)propan-2-ol;
2-(4-(1-phenyl-2,3-dihydro-1H-benzo[d]pyrrolo[1,2-a]imidazol-7-yl)phenyl)acetonitrile;
4-(5-(8-phenyl-7,8-dihydro-6H-pyrrolo[2′,1′:2,3]imidazo[4,5-b]pyridin-2-yl)pyrimidin-2-yl)piperazin-2-one;
7-(2-cyclopropylpyrimidin-5-yl)-1-phenyl-2,3-dihydro-1H-benzo[d]pyrrolo[1,2-a]imidazole;
(S)-4-(5-(4-phenyl-3,4-dihydro-1H-benzo[4,5]imidazo[2,1-c][1,4]oxazin-7-yl)pyrimidin-2-yl)piperazin-2-one;
2-((5-(9-(2-methoxyphenyl)-6,7,8,9-tetrahydrobenzo[4,5]imidazo[1,2-a]pyridin-2-yl)pyridin-2-yl)oxy)acetic acid;
7-(6-(ethylsulfonyl)pyridin-3-yl)-1-phenyl-2,3-dihydro-1H-benzo[d]pyrrolo[1,2-a]imidazole;
4-(5-(8-(3-fluorophenyl)-7,8-dihydro-6H-pyrrolo[2′,1′:2,3]imidazo[4,5-b]pyridin-2-yl)pyrimidin-2-yl)piperazin-2-one;
10-(3-fluorophenyl)-2-(2-morpholinopyrimidin-5-yl)-7,8,9,10-tetrahydro-6H-cyclohepta[4,5]imidazo[1,2-a]pyridin-10-ol;
4-(5-(9-phenyl-6,7,8,9-tetrahydroimidazo[1,2-a:5,4-b′]dipyridin-2-yl)pyrimidin-2-yl)piperazin-2-one;
(S)-6-(5-(4-phenyl-3,4-dihydro-1H-benzo[4,5]imidazo[2,1-c][1,4]oxazin-7-yl)pyrimidin-2-yl)-6-azaspiro[3.4]octan-2-ol;
N,N-dimethyl-4-(1-phenyl-2,3-dihydro-1H-benzo[d]pyrrolo[1,2-a]imidazol-7-yl)benzamide;
N-ethyl-N-methyl-4-(1-phenyl-2,3-dihydro-1H-benzo[d]pyrrolo[1,2-a]imidazol-7-yl)benzamide;
7-(6-morpholinopyridin-3-yl)-4-phenyl-3,4-dihydro-1H-benzo[4,5]imidazo[2,1-c][1,4]oxazine;
2-(5-(1-phenyl-1,2,3,4-tetrahydrobenzo[4,5]imidazo[1,2-a]pyridin-8-yl)pyrimidin-2-yl)propan-2-ol;
2-(5-(1-Phenyl-2,3-dihydro-1H-benzo[d]pyrrolo[1,2-a]imidazol-7-yl)pyrimidin-2-yl)propan-2-ol;
(S)-4-(2-hydroxyethyl)-1-(5-(4-phenyl-3,4-dihydro-1H-benzo[4,5]imidazo[2,1-c][1,4]oxazin-7-yl)pyrimidin-2-yl)piperidin-4-ol;
(S)-7-(2-(2-oxa-7-azaspiro[3.5]nonan-7-yl)pyrimidin-5-yl)-4-phenyl-3,4-dihydro-1H-benzo[4,5]imidazo[2,1-c][1,4]oxazine;
2-(1-(5-(S)-4-phenyl-3,4-dihydro-1H-benzo[4,5]imidazo[2,1-c][1,4]oxazin-7-yl)pyrimidin-2-yl)piperidin-3-yl)acetic acid;
7-(5-methyl-6-morpholinopyridin-3-yl)-4-phenyl-3,4-dihydro-1H-benzo[4,5]imidazo[2,1-c][1,4]oxazine;
7-(5-(2-methyl-1H-imidazol-1-yl)pyrazin-2-yl)-4-phenyl-3,4-dihydro-1H-benzo[4,5]imidazo[2,1-c][1,4]oxazine;
2-(5-(1-cyclohexyl-2,3-dihydro-1H-benzo[d]pyrrolo[1,2-a]imidazol-7-yl)pyrimidin-2-yl)propan-2-ol;
2-(5-(4-phenyl-3,4-dihydro-1H-benzo[4,5]imidazo[2,1-c][1,4]oxazin-7-yl)pyrimidin-2-yl)propan-2-ol;
(S)-(4-(methylsulfonyl)-1-(5-(4-phenyl-3,4-dihydro-1H-benzo[4,5]imidazo[2,1-c][1,4]oxazin-7-yl)pyrimidin-2-yl)piperidin-4-yl)methanol;
1-(1-(5-((S)-4-phenyl-3,4-dihydro-1H-benzo[4,5]imidazo[2,1-c][1,4]oxazin-7-yl)pyrimidin-2-yl)piperidin-4-yl)ethanol;
(S)-4-(5-(4-phenyl-3,4-dihydro-1H-benzo[4,5]imidazo[2,1-c][1,4]oxazin-7-yl)pyrimidin-2-yl)-1,4-diazepan-2-one;
2-(4-(1-phenyl-2,3-dihydro-1H-benzo[d]pyrrolo[1,2-a]imidazol-7-yl)phenoxy)acetonitrile;
(S)—N-(1-(5-(4-phenyl-3,4-dihydro-1H-benzo[4,5]imidazo[2,1-c][1,4]oxazin-7-yl)pyrimidin-2-yl)piperidin-4-yl)methanesulfonamide;
(S)-3-(1-(5-(4-phenyl-3,4-dihydro-1H-benzo[4,5]imidazo[2,1-c][1,4]oxazin-7-yl)pyrimidin-2-yl)piperidin-4-yl)propanoic acid;
4-phenyl-7-(6-(trifluoromethyl)pyridin-3-yl)-3,4-dihydro-1H-benzo[4,5]imidazo[2,1-c][1,4]oxazine;
4-(5-(1-phenyl-1,2,3,4-tetrahydrobenzo[4,5]imidazo[1,2-a]pyridin-8-yl)pyrimidin-2-yl)piperazin-2-one;
7-(5-fluoro-6-methoxypyridin-3-yl)-4-phenyl-3,4-dihydro-1H-benzo[4,5]imidazo[2,1-c][1,4]oxazine;
N,N-dimethyl-5-(1-phenyl-2,3-dihydro-1H-benzo[d]pyrrolo[1,2-a]imidazol-7-yl)pyridin-2-amine;
7-(2-methylpyridin-4-yl)-1-phenyl-2,3-dihydro-1H-benzo[d]pyrrolo[1,2-a]imidazole;
(4S)-4-phenyl-7-(2-(2-(trifluoromethyl)morpholino)pyrimidin-5-yl)-3,4-dihydro-1H-benzo[4,5]imidazo[2,1-c][1,4]oxazine;
7-(5-((S)-4-phenyl-3,4-dihydro-1H-benzo[4,5]imidazo[2,1-c][1,4]oxazin-7-yl)pyrimidin-2-yl)-2,7-diazaspiro[4.4]nonan-1-one;
N-cyclopentyl-5-(4-phenyl-3,4-dihydro-1H-benzo[4,5]imidazo[2,1-c][1,4]oxazin-7-yl)pyrimidin-2-amine;
7-(2-(1H-pyrazol-1-yl)pyrimidin-5-yl)-4-phenyl-3,4-dihydro-1H-benzo[4,5]imidazo[2,1-c][1,4]oxazine;
(4S)-7-(2-(2,6-dimethylmorpholino)pyrimidin-5-yl)-4-phenyl-3,4-dihydro-1H-benzo[4,5]imidazo[2,1-c][1,4]oxazine;
7-(6-methylpyridin-3-yl)-1-phenyl-2,3-dihydro-1H-benzo[d]pyrrolo[1,2-a]imidazole;
7-(5-ethoxypyridin-3-yl)-1-phenyl-2,3-dihydro-1H-benzo[d]pyrrolo[1,2-a]imidazole;
2-(2-morpholinopyrimidin-5-yl)-9-(m-tolyl)-6,7,8,9-tetrahydrobenzo[4,5]imidazo[1,2-a]pyridin-9-ol;
7-(6-(methylthio)pyridin-3-yl)-1-phenyl-2,3-dihydro-1H-benzo[d]pyrrolo[1,2-a]imidazole;
ethyl 2-((5-(10-(2-methoxyphenyl)-7,8,9,10-tetrahydro-6H-cyclohepta[4,5]imidazo[1,2-a]pyridin-2-yl)pyrimidin-2-yl)amino)acetate;
(S)-3-(4-(5-(4-phenyl-3,4-dihydro-1H-benzo[4,5]imidazo[2,1-c][1,4]oxazin-7-yl)pyrimidin-2-yl)piperazin-1-yl)propan-1-ol;
9-(3-fluorophenyl)-2-(2-morpholinopyrimidin-5-yl)-6,7,8,9-tetrahydrobenzo[4,5]imidazo[1,2-a]pyridin-9-ol;
2-(2-morpholinopyrimidin-5-yl)-9-phenyl-6,7,8,9-tetrahydrobenzo[4,5]imidazo[1,2-a]pyridin-9-ol;
4-(2,5-difluorophenyl)-7-(2-morpholinopyrimidin-5-yl)-3,4-dihydro-1H-benzo[4,5]imidazo[2,1-c][1,4]oxazine;
1-phenyl-7-(6-(piperazin-1-yl)pyridin-3-yl)-2,3-dihydro-1H-benzo[d]pyrrolo[1,2-a]imidazole;
9-(2-Methoxyphenyl)-2-(2-morpholinopyrimidin-5-yl)-6,7,8,9-tetrahydrobenzo[4,5]imidazo[1,2-a]pyridin-9-ol;
(S)-7-(2-(2-oxa-6-azaspiro[3.4]octan-6-yl)pyrimidin-5-yl)-4-phenyl-3,4-dihydro-1H-benzo[4,5]imidazo[2,1-c][1,4]oxazine;
7-(5-(1H-imidazol-1-yl)pyrazin-2-yl)-4-phenyl-3,4-dihydro-1H-benzo[4,5]imidazo[2,1-c][1,4]oxazine;
7-(furo[3,2-b]pyridin-6-yl)-4-phenyl-3,4-dihydro-1H-benzo[4,5]imidazo[2,1-c][1,4]oxazine;
10-(3-chlorophenyl)-2-(2-morpholinopyrimidin-5-yl)-7,8,9,10-tetrahydro-6H-cyclohepta[4,5]imidazo[1,2-a]pyridin-10-ol;
N-ethyl-4-(1-phenyl-2,3-dihydro-1H-benzo[d]pyrrolo[1,2-a]imidazol-7-yl)benzamide;
2-(3-(1-phenyl-2,3-dihydro-1H-benzo[d]pyrrolo[1,2-a]imidazol-7-yl)phenoxy)acetonitrile;
2-(2-morpholinopyrimidin-5-yl)-10-(m-tolyl)-7,8,9,10-tetrahydro-6H-cyclohepta[4,5]imidazo[1,2-a]pyridin-10-ol;
2-((5-(10-(2-methoxyphenyl)-7,8,9,10-tetrahydro-6H-cyclohepta[4,5]imidazo[1,2-a]pyridin-2-yl)pyrimidin-2-yl)amino)acetic acid;
N-cyclopropyl-4-(1-phenyl-2,3-dihydro-1H-benzo[d]pyrrolo[1,2-a]imidazol-7-yl)benzamide;
4-(I-phenyl-2,3-dihydro-1H-benzo[d]pyrrolo[1,2-a]imidazol-7-yl)benzamide;
1-(4-(5-(4-phenyl-3,4-dihydro-1H-benzo[4,5]imidazo[2,1-c][1,4]oxazin-7-yl)pyridin-2-yl)piperazin-1-yl)ethanone;
7-(6-(4-methylpiperazin-1-yl)pyridin-3-yl)-1-phenyl-2,3-dihydro-1H-benzo[d]pyrrolo[1,2-a]imidazole;
7-(benzo[d][1,3]dioxol-5-yl)-1-phenyl-2,3-dihydro-1H-benzo[d]pyrrolo[1,2-a]imidazole;
9-(3-fluoro-2-methylphenyl)-2-(2-morpholinopyrimidin-5-yl)-6,7,8,9-tetrahydrobenzo[4,5]imidazo[1,2-a]pyridin-9-ol;
8-Phenyl-2-(4-(pyrimidin-2-yl)piperazin-1-yl)-7,8-dihydro-6H-pyrrolo[2′,1′:2,3]imidazo[4,5-b]pyridine;
9-(4-fluorophenyl)-2-(2-morpholinopyrimidin-5-yl)-6,7,8,9-tetrahydrobenzo[4,5]imidazo[1,2-a]pyridin-9-ol;
7-(5-((S)-4-phenyl-3,4-dihydro-1H-benzo[4,5]imidazo[2,1-c][1,4]oxazin-7-yl)pyrimidin-2-yl)tetrahydro-1H-oxazolo[3,4-a]pyrazin-3(5H)-one;
2-((5-(9-(2-methoxyphenyl)-6,7,8,9-tetrahydrobenzo[4,5]imidazo[1,2-a]pyridin-2-yl)pyrimidin-2-yl)amino)acetic acid;
10-(4-fluorophenyl)-2-(2-morpholinopyrimidin-5-yl)-7,8,9,10-tetrahydro-6H-cyclohepta[4,5]imidazo[1,2-a]pyridin-10-ol;
9-(3-chlorophenyl)-2-(2-morpholinopyrimidin-5-yl)-6,7,8,9-tetrahydrobenzo[4,5]imidazo[1,2-a]pyridin-9-ol;
N-cyclopropyl-5-(4-phenyl-3,4-dihydro-1H-benzo[4,5]imidazo[2,1-c][1,4]oxazin-7-yl)pyrimidin-2-amine;
9-(3-chloro-5-fluorophenyl)-2-(2-morpholinopyrimidin-5-yl)-6,7,8,9-tetrahydrobenzo[4,5]imidazo[1,2-a]pyridin-9-ol;
N,N-dimethyl-5-(4-phenyl-3,4-dihydro-1H-benzo[4,5]imidazo[2,1-c][1,4]oxazin-7-yl)pyrimidin-2-amine;
1-(5-(1-phenyl-1,2,3,4-tetrahydrobenzo[4,5]imidazo[1,2-a]pyridin-8-yl)pyrimidin-2-yl)piperidine-4-carboxylic acid;
7-(6-isopropoxypyridin-3-yl)-4-phenyl-3,4-dihydro-1H-benzo[4,5]imidazo[2,1-c][1,4]oxazine;
7-(6-isopropoxypyridin-3-yl)-4-phenyl-3,4-dihydro-1H-benzo[4,5]imidazo[2,1-c][1,4]oxazine;
4-(5-(6-phenyl-7,8-dihydro-6H-pyrrolo[1′,2′:1,2]imidazo[4,5-c]pyridin-3-yl)pyrimidin-2-yl)morpholine;
1-phenyl-7-(1-(pyridin-3-ylmethyl)-1H-pyrazol-4-yl)-2,3-dihydro-1H-benzo[d]pyrrolo[1,2-a]imidazole;
5-(4-phenyl-3,4-dihydro-1H-benzo[4,5]imidazo[2,1-c][1,4]oxazin-7-yl)picolinonitrile;
7-(4-methyl-3,4-dihydro-2H-pyrido[3,2-b][1,4]oxazin-7-yl)-4-phenyl-3,4-dihydro-1H-benzo[4,5]imidazo[2,1-c][1,4]oxazine;
2-(2-morpholinopyrimidin-5-yl)-9-(p-tolyl)-6,7,8,9-tetrahydrobenzo[4,5]imidazo[1,2-a]pyridin-9-ol;
7-(6-(methylsulfonyl)pyridin-3-yl)-4-phenyl-3,4-dihydro-1H-benzo[4,5]imidazo[2,1-c][1,4]oxazine;
N-(2-methoxyethyl)-4-(1-phenyl-2,3-dihydro-1H-benzo[d]pyrrolo[1,2-a]imidazol-7-yl)benzamide;
N-methyl-4-(1-phenyl-2,3-dihydro-1H-benzo[d]pyrrolo[1,2-a]imidazol-7-yl)benzamide;
4-phenyl-7-(6-(2,2,2-trifluoroethoxy)pyridin-3-yl)-3,4-dihydro-1H-benzo[4,5]imidazo[2,1-c][1,4]oxazine;
1-(5-(4-phenyl-3,4-dihydro-1H-benzo[4,5]imidazo[2,1-c][1,4]oxazin-7-yl)pyrimidin-2-yl)piperidine-4-carboxylic acid;
7-(5-methyl-6-(4-methylpiperazin-1-yl)pyridin-3-yl)-4-phenyl-3,4-dihydro-1H-benzo[4,5]imidazo[2,1-c][1,4]oxazine;
5-(9-(2-Methoxyphenyl)-6,7,8,9-tetrahydrobenzo[4,5]imidazo[1,2-a]pyridin-2-yl)thiophene-2-carboxylic acid;
7-(6-(4-methylpiperazin-1-yl)pyridin-3-yl)-4-phenyl-3,4-dihydro-1H-benzo[4,5]imidazo[2,1-c][1,4]oxazine;
(S)-7-(2-(1′-methyl-[4,4′-bipiperidin]-1-yl)pyrimidin-5-yl)-4-phenyl-3,4-dihydro-1H-benzo[4,5]imidazo[2,1-c][1,4]oxazine;
7-(2-methoxypyrimidin-5-yl)-4-phenyl-3,4-dihydro-1H-benzo[4,5]imidazo[2,1-c][1,4]oxazine;
10-(3-chloro-5-fluorophenyl)-2-(2-morpholinopyrimidin-5-yl)-7,8,9,10-tetrahydro-6H-cyclohepta[4,5]imidazo[1,2-a]pyridin-10-ol;
3-(2-hydroxyethyl)-1-(5-((S)-4-phenyl-3,4-dihydro-1H-benzo[4,5]imidazo[2,1-c][1,4]oxazin-7-yl)pyrimidin-2-yl)pyrrolidin-3-ol;
4-(5-(9-(2-Methoxyphenyl)-6,7-dihydrobenzo[4,5]imidazo[1,2-a]pyridin-2-yl)pyrimidin-2-yl)morpholine;
10-(4-methoxyphenyl)-2-(2-morpholinopyrimidin-5-yl)-7,8,9,10-tetrahydro-6H-cyclohepta[4,5]imidazo[1,2-a]pyridin-10-ol;
7-(5-(1H-pyrazol-1-yl)pyrazin-2-yl)-4-phenyl-3,4-dihydro-1H-benzo[4,5]imidazo[2,1-c][1,4]oxazine;
1-phenyl-7-(pyridin-3-yl)-2,3-dihydro-1H-benzo[d]pyrrolo[1,2-a]imidazole;
5-(1-phenyl-2,3-dihydro-1H-benzo[d]pyrrolo[1,2-a]imidazol-7-yl)pyrimidin-2-amine;
2-(2-morpholinopyrimidin-5-yl)-10-(p-tolyl)-7,8,9,10-tetrahydro-6H-cyclohepta[4,5]imidazo[1,2-a]pyridin-10-ol;
5-(4-phenyl-3,4-dihydro-1H-benzo[4,5]imidazo[2,1-c][1,4]oxazin-7-yl)pyrimidine-2-carbonitrile;
(R)-2-(5-(9-(2-methoxyphenyl)-6,7,8,9-tetrahydrobenzo[4,5]imidazo[1,2-a]pyridin-2-yl)pyrimidin-2-yl)propan-2-ol;
(S)-7-(2-((R)-3-(methylsulfonyl)pyrrolidin-1-yl)pyrimidin-5-yl)-4-phenyl-3,4-dihydro-1H-benzo[4,5]imidazo[2,1-c][1,4]oxazine;
7-([1,2,5]oxadiazolo[3,4-b]pyridin-6-yl)-4-phenyl-3,4-dihydro-1H-benzo[4,5]imidazo[2,1-c][1,4]oxazine;
2-(5-(6-phenyl-7,8-dihydro-6H-pyrrolo[1,2′:1,2]imidazo[4,5-c]pyridin-3-yl)pyrimidin-2-yl)propan-2-ol;
7-(2-methylpyrimidin-5-yl)-4-phenyl-3,4-dihydro-1H-benzo[4,5]imidazo[2,1-c][1,4]oxazine;
1-phenyl-7-(pyrimidin-5-yl)-2,3-dihydro-1H-benzo[d]pyrrolo[1,2-a]imidazole;
7-(6-methoxy-5-methylpyridin-3-yl)-4-phenyl-3,4-dihydro-1H-benzo[4,5]imidazo[2,1-c][1,4]oxazine;
10-(4-chlorophenyl)-2-(2-morpholinopyrimidin-5-yl)-7,8,9,10-tetrahydro-6H-cyclohepta[4,5]imidazo[1,2-a]pyridin-10-ol;
2-(3-(1-phenyl-2,3-dihydro-1H-benzo[d]pyrrolo[1,2-a]imidazol-7-yl)phenyl)acetonitrile;
N-(3-(1-phenyl-2,3-dihydro-1H-benzo[d]pyrrolo[1,2-a]imidazol-7-yl)benzyl)methanesulfonamide;
7-(6-methylpyridin-3-yl)-4-phenyl-3,4-dihydro-1H-benzo[4,5]imidazo[2,1-c][1,4]oxazine;
(S)-2-(5-(1-phenyl-1,2,3,4-tetrahydrobenzo[4,5]imidazo[1,2-a]pyridin-8-yl)pyrimidin-2-yl)propan-2-ol;
(S)-7-(2-(4-(1-methylpiperidin-4-yl)piperazin-1-yl)pyrimidin-5-yl)-4-phenyl-3,4-dihydro-1H-benzo[4,5]imidazo[2,1-c][1,4]oxazine;
4-phenyl-7-(6-(piperazin-1-yl)pyridin-3-yl)-3,4-dihydro-1H-benzo[4,5]imidazo[2,1-c][1,4]oxazine;
(4S)-7-(2-(3-(methylsulfonyl)pyrrolidin-1-yl)pyrimidin-5-yl)-4-phenyl-3,4-dihydro-1H-benzo[4,5]imidazo[2,1-c][1,4]oxazine;
(S)-8-(5-(4-phenyl-3,4-dihydro-1H-benzo[4,5]imidazo[2,1-c][1,4]oxazin-7-yl)pyrimidin-2-yl)-1,3,8-triazaspiro[4.5]decan-4-one;
7-(6-isopropoxy-5-methylpyridin-3-yl)-4-phenyl-3,4-dihydro-1H-benzo[4,5]imidazo[2,1-c][1,4]oxazine;
7-(5-methylpyridin-3-yl)-1-phenyl-2,3-dihydro-H-benzo[d]pyrrolo[1,2-a]imidazole;
N-methyl-3-(1-phenyl-2,3-dihydro-1H-benzo[d]pyrrolo[1,2-a]imidazol-7-yl)benzamide;
4-(1-phenyl-2,3-dihydro-1H-benzo[d]pyrrolo[1,2-a]imidazol-7-yl)-N-((tetrahydrofuran-2-yl)methyl)benzamide;
(S)-4-(5-(1-phenyl-2,3-dihydro-1H-benzo[d]pyrrolo[1,2-a]imidazol-7-yl)pyrimidin-2-yl)morpholine;
N-(5-(4-phenyl-3,4-dihydro-1H-benzo[4,5]imidazo[2,1-c][1,4]oxazin-7-yl)pyridin-2-yl)acetamide;
N-ethyl-N-methyl-3-(1-phenyl-2,3-dihydro-1H-benzo[d]pyrrolo[1,2-a]imidazol-7-yl)benzamide;
5-(1-phenyl-2,3-dihydro-1H-benzo[d]pyrrolo[1,2-a]imidazol-7-yl)pyridin-2-amine;
7-(3-methyl-3H-imidazo[4,5-b]pyridin-6-yl)-4-phenyl-3,4-dihydro-1H-benzo[4,5]imidazo[2,1-c][1,4]oxazine;
10-(3,5-dimethoxyphenyl)-2-(2-morpholinopyrimidin-5-yl)-7,8,9,10-tetrahydro-6H-cyclohepta[4,5]imidazo[1,2-a]pyridin-10-ol;
N,N-dimethyl-3-((5-(4-phenyl-3,4-dihydro-1H-benzo[4,5]imidazo[2,1-c][1,4]oxazin-7-yl)pyridin-2-yl)oxy)propan-1-amine;
(3R,4R)-1-(5-((S)-4-phenyl-3,4-dihydro-1H-benzo[4,5]imidazo[2,1-c][1,4]oxazin-7-yl)pyrimidin-2-yl)pyrrolidine-3,4-diol;
N-(2-(dimethylamino)ethyl)-3-(1-phenyl-2,3-dihydro-1H-benzo[d]pyrrolo[1,2-a]imidazol-7-yl)benzamide;
10-(3-methoxyphenyl)-2-(2-morpholinopyrimidin-5-yl)-7,8,9,10-tetrahydro-6H-cyclohepta[4,5]imidazo[1,2-a]pyridin-10-ol;
7-(1,5-dimethyl-1H-pyrazol-4-yl)-1-phenyl-2,3-dihydro-1H-benzo[d]pyrrolo[1,2-a]imidazole;
N,N-dimethyl-3-(1-phenyl-2,3-dihydro-1H-benzo[d]pyrrolo[1,2-a]imidazol-7-yl)benzamide;
7-(3-(methylsulfonyl)phenyl)-1-phenyl-2,3-dihydro-1H-benzo[d]pyrrolo[1,2-a]imidazole;
4-phenyl-7-(pyrido[2,3-b]pyrazin-7-yl)-3,4-dihydro-1H-benzo[4,5]imidazo[2,1-c][1,4]oxazine;
1-methyl-5-(1-phenyl-2,3-dihydro-1H-benzo[d]pyrrolo[1,2-a]imidazol-7-yl)pyridin-2(1H)-one;
(3S,4S)-1-(5-((S)-4-phenyl-3,4-dihydro-1H-benzo[4,5]imidazo[2,1-c][1,4]oxazin-7-yl)pyrimidin-2-yl)pyrrolidine-3,4-diol;
4-phenyl-7-(pyrimidin-5-yl)-3,4-dihydro-1H-benzo[4,5]imidazo[2,1-c][1,4]oxazine;
(S)-2-(5-(1-(2-Methoxyphenyl)-2,3-dihydro-1H-cyclopenta[4,5]imidazo[1,2-a]pyridin-7-yl)pyrimidin-2-yl)propan-2-ol;
(S)-2-(5-(1-phenyl-2,3-dihydro-1H-benzo[d]pyrrolo[1,2-a]imidazol-7-yl)pyrimidin-2-yl)propan-2-ol;
2-(2-(2-(2-hydroxypropan-2-yl)pyrimidin-5-yl)-6,7,8,9-tetrahydrobenzo[4,5]imidazo[1,2-a]pyridin-9-yl)phenol;
4-(5-(6-phenyl-6,7,8,9-tetrahydroimidazo[1,2-a:4,5-b′]dipyridin-3-yl)pyrimidin-2-yl)piperazin-2-one;
(S)-7-(2-(3-morpholinoazetidin-1-yl)pyrimidin-5-yl)-4-phenyl-3,4-dihydro-1H-benzo[4,5]imidazo[2,1-c][1,4]oxazine;
7-(5-(methylsulfonyl)pyridin-3-yl)-1-phenyl-2,3-dihydro-1H-benzo[d]pyrrolo[1,2-a]imidazole;
(R)-7-(2-Morpholinopyrimidin-5-yl)-4-phenyl-3,4-dihydro-1H-benzo[4,5]imidazo[2,1-c][1,4]oxazine;
7-(2-methylpyridin-3-yl)-1-phenyl-2,3-dihydro-1H-benzo[d]pyrrolo[1,2-a]imidazole;
1-(5-(1-phenyl-1,2,3,4-tetrahydrobenzo[4,5]imidazo[1,2-a]pyridin-8-yl)pyrimidin-2-yl)azetidine-3-carboxylic acid;
7-(1-methyl-1H-pyrrol-3-yl)-1-phenyl-2,3-dihydro-1H-benzo[d]pyrrolo[1,2-a]imidazole; or
N-(2-morpholinoethyl)-5-(4-phenyl-3,4-dihydro-1H-benzo[4,5]imidazo[2,1-c][1,4]oxazin-7-yl)pyridin-2-amine.
In certain embodiments, the compound is selected from the group consisting of:
7-(5-((R)-1-Phenyl-2,3-dihydro-1H-benzo[d]pyrrolo[1,2-a]imidazol-7-yl)pyrimidin-2-yl)hexahydroimidazo[1,5-a]pyrazin-3(2H)-one;
3,3-difluoro-1-(5-((R)-1-phenyl-2,3-dihydro-1H-benzo[d]pyrrolo[1,2-a]imidazol-7-yl)pyrimidin-2-yl)piperidin-4-ol;
(S)-2-(5-(4-(2-(Difluoromethoxy)phenyl)-3,4-dihydro-1H-benzo[4,5]imidazo[2,1-c][1,4]oxazin-7-yl)pyrimidin-2-yl)propan-2-ol;
(S)-2-(5-(9-(2-methoxyphenyl)-6,7,8,9-tetrahydrobenzo[4,5]imidazo[1,2-a]pyridin-2-yl)pyrimidin-2-yl)propan-2-ol;
(R)-7-(5-(1-phenyl-2,3-dihydro-1H-benzo[d]pyrrolo[1,2-a]imidazol-7-yl)pyrimidin-2-yl)-7-azaspiro[3.5]nonan-2-ol;
1-(5-(8-(2,5-dimethylphenyl)-7,8-dihydro-6H-pyrrolo[2′,1′:2,3]imidazo[4,5-b]pyridin-2-yl)pyrimidin-2-yl)piperidin-4-ol;
4-(5-(10-(2-methoxyphenyl)-7,8,9,10-tetrahydro-6H-cyclohepta[4,5]imidazo[1,2-a]pyridin-2-yl)pyrimidin-2-yl)morpholine;
(R)-4-(5-(1-phenyl-2,3-dihydro-1H-benzo[d]pyrrolo[1,2-a]imidazol-7-yl)pyrimidin-2-yl)morpholine;
7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one;
4-(5-(8-(2,5-dimethylphenyl)-7,8-dihydro-6H-pyrrolo[2′,1′:2,3]imidazo[4,5-b]pyridin-2-yl)pyrimidin-2-yl)piperazin-2-one;
2-(5-(1-(2,5-dimethylphenyl)-2,3-dihydro-1H-benzo[d]pyrrolo[1,2-a]imidazol-7-yl)pyrimidin-2-yl)propan-2-ol;
3,3-difluoro-1-(5-((S)-4-phenyl-3,4-dihydro-1H-benzo[4,5]imidazo[2,1-c][1,4]oxazin-7-yl)pyrimidin-2-yl)piperidin-4-ol;
7-(5-(4-(3-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one;
(R)-2-(5-(1-(2-methoxyphenyl)-2,3-dihydro-1H-cyclopenta[4,5]imidazo[1,2-a]pyridin-7-yl)pyrimidin-2-yl)propan-2-ol;
7-(4-(isopropylsulfonyl)phenyl)-1-phenyl-2,3-dihydro-1H-benzo[d]pyrrolo[1,2-a]imidazole;
2-(5-(9-(2-Methoxyphenyl)-6,7,8,9-tetrahydrobenzo[4,5]imidazo[1,2-a]pyridin-2-yl)pyrimidin-2-yl)propan-2-ol;
4-(5-(8-(2,5-dimethylphenyl)-7,8-dihydro-6H-pyrrolo[2′,1′:2,3]imidazo[4,5-b]pyridin-2-yl)pyrimidin-2-yl)morpholine;
(S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol;
4-(5-(9-(2-methoxyphenyl)-6,7,8,9-tetrahydrobenzo[4,5]imidazo[1,2-a]pyridin-2-yl)pyrimidin-2-yl)morpholine;
1-(5-(9-phenyl-6,7,8,9-tetrahydroimidazo[1,2-a:5,4-b′]dipyridin-2-yl)pyrimidin-2-yl)piperidin-4-ol;
(R)-2-(5-(1-Phenyl-2,3-dihydro-1H-benzo[d]pyrrolo[1,2-a]imidazol-7-yl)pyrimidin-2-yl)propan-2-ol;
2-(5-(10-(2-methoxyphenyl)-7,8,9,10-tetrahydro-6H-cyclohepta[4,5]imidazo[1,2-a]pyridin-2-yl)pyrimidin-2-yl)propan-2-ol;
N-methyl-4-(1-phenyl-2,3-dihydro-1H-benzo[d]pyrrolo[1,2-a]imidazol-7-yl)benzenesulfonamide;
(S)-7-(2-morpholinopyrimidin-5-yl)-4-phenyl-3,4-dihydro-1H-benzo[4,5]imidazo[2,1-c][1,4]oxazine;
(S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol;
2-(5-(1-(2-methoxyphenyl)-2,3-dihydro-1H-cyclopenta[4,5]imidazo[1,2-a]pyridin-7-yl)pyrimidin-2-yl)propan-2-ol;
(R)-2-(5-(1-phenyl-1,2,3,4-tetrahydrobenzo[4,5]imidazo[1,2-a]pyridin-8-yl)pyrimidin-2-yl)propan-2-ol;
ethyl 2-[[5-[9-(2-methoxyphenyl)-6,7,8,9-tetrahydropyrido[1,2-a]benzimidazol-2-yl]pyrimidin-2-yl]amino]acetate.r
2-((5-(9-(2-methoxyphenyl)-6,7,8,9-tetrahydrobenzo[4,5]imidazo[1,2-a]pyridin-2-yl)pyridin-2-yl)oxy)acetic acid.

In certain embodiments, the compound is selected from the group consisting of:
(8aR)-7-(5-(6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3(2H)-one;
3-((5-(6′,8′-dihydro-2H-spiro[benzofuran-3,9′-pyrido[3′,2′:4,5]imidazo[2,1-c][1,4]oxazin]-2′-yl)pyrimidin-2-yl)amino)cyclobutanol;
5-(6′,8′-dihydro-2H-spiro[benzofuran-3,9′-pyrido[3′,2′:4,5]imidazo[2,1-c][1,4]oxazin]-2′-yl)-N-(tetrahydrofuran-3-yl)pyrimidin-2-amine;
1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-3-ol;
2′-(2-(4-methylpiperazin-1-yl)pyrimidin-5-yl)-6′,8′-dihydro-2H-spiro[benzofuran-3,9′-pyrido[3′,2′:4,5]imidazo[2,1-c][1,4]oxazine];
2′-(2-morpholinopyrimidin-5-yl)-6′,8′-dihydro-2H-spiro[benzofuran-3,9′-pyrido[3′,2′:4,5]imidazo[2,1-c][1,4]oxazine];
1-(5-(1,2′,3,3′-tetrahydrospiro[benzo[4,5]imidazo[2,1-c][1,4]oxazine-4,1′-inden]-7-yl)pyrimidin-2-yl)piperidin-4-ol;
(1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-3-yl)methanol;
(8aS)-7-(5-(2,3,6′,8′-tetrahydrospiro[indene-1,9′-pyrido[3′,2′:4,5]imidazo[2,1-c][1,4]oxazin]-2′-yl)pyrimidin-2-yl)hexahydroimidazo[1,5-a]pyrazin-3(2H)-one;
2′-(2-(1,4-oxazepan-4-yl)pyrimidin-5-yl)-2,3,6′,8′-tetrahydrospiro[indene-1,9′-pyrido[3′,2′:4,5]imidazo[2,1-c][1,4]oxazine];
3,3-difluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-ol;
2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)propan-1-one;
2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)ethanone;
(4-fluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-yl)methanol;
2′-(2-morpholinopyrimidin-5-yl)-2,3,6′,8′-tetrahydrospiro[indene-1,9′-pyrido[3′,2′:4,5]imidazo[2,1-c][1,4]oxazine];
1-(5-(2,3,6′,8′-tetrahydrospiro[indene-1,9′-pyrido[3′,2′:4,5]imidazo[2,1-c][1,4]oxazin]-2′-yl)pyrimidin-2-yl)azepan-4-ol;
(S)-2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol;
(R)-2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol;
(8aR)-7-(5-(6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3(2H)-one;
3-((5-(6′,8′-dihydro-2H-spiro[benzofuran-3,9′-pyrido[3′,2′:4,5]imidazo[2,1-c][1,4]oxazin]-2′-yl)pyrimidin-2-yl)amino)cyclobutanol;
5-(6′,8′-dihydro-2H-spiro[benzofuran-3,9′-pyrido[3′,2′:4,5]imidazo[2,1-c][1,4]oxazin]-2′-yl)-N-(tetrahydrofuran-3-yl)pyrimidin-2-amine;
1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-3-ol;
2′-(2-(4-methylpiperazin-1-yl)pyrimidin-5-yl)-6′,8′-dihydro-2H-spiro[benzofuran-3,9′-pyrido[3′,2′:4,5]imidazo[2,1-c][1,4]oxazine];
2′-(2-morpholinopyrimidin-5-yl)-6′,8′-dihydro-2H-spiro[benzofuran-3,9′-pyrido[3′,2′:4,5]imidazo[2,1-c][1,4]oxazine];
1-(5-(1,2′,3,3′-tetrahydrospiro[benzo[4,5]imidazo[2,1-c][1,4]oxazine-4,1′-inden]-7-yl)pyrimidin-2-yl)piperidin-4-ol;
(1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-3-yl)methanol;
(8aR)-7-(5-(2,3,6′,8′-tetrahydrospiro[indene-1,9′-pyrido[3′,2′:4,5]imidazo[2,1-c][1,4]oxazin]-2′-yl)pyrimidin-2-yl)hexahydroimidazo[1,5-a]pyrazin-3(2H)-one;
2′-(2-(1,4-oxazepan-4-yl)pyrimidin-5-yl)-2,3,6′,8′-tetrahydrospiro[indene-1,9′-pyrido[3′,2′:4,5]imidazo[2,1-c][1,4]oxazine];
3,3-difluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-ol;
2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)propan-1-one;
2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)ethanone;
(4-fluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-yl)methanol;
2′-(2-morpholinopyrimidin-5-yl)-2,3,6′,8′-tetrahydrospiro[indene-1,9′-pyrido[3′,2′:4,5]imidazo[2,1-c][1,4]oxazine];
1-(5-(2,3,6′,8′-tetrahydrospiro[indene-1,9′-pyrido[3′,2′:4,5]imidazo[2,1-c][1,4]oxazin]-2′-yl)pyrimidin-2-yl)azepan-4-ol;
(R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2(1H)-one:
(1r,4r)-4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol;
(1s,4s)-4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol;
3-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclopentanol;
2′-(2-morpholinopyrimidin-5-yl)-6′,7′-dihydrospiro[cyclohexane-1,9′-pyrano[4′,3′:4,5]imidazo[1,2-b]pyridazine];
2′-(2-morpholinopyrimidin-5-yl)-6′,7′-dihydrospiro[chroman-4,9′-pyrano[4′,3′:4,5]imidazo[1,2-b]pyridazine];
2′-(2-(piperazin-1-yl)pyrimidin-5-yl)-6′H,8′H-spiro[chromane-4,9′-pyrido[3′,2′:4,5]imidazo[2,1-c][1,4]oxazine];
2′-(2-methoxypyrimidin-5-yl)-6′,8′-dihydrospiro[chroman-4,9′-pyrido[3,2′:4,5]imidazo[2,1-c][1,4]oxazine];
2′-(2-ethoxypyrimidin-5-yl)-6′,8-dihydrospiro[chroman-4,9′-pyrido[3,2′:4,5]imidazo[2,1-c][1,4]oxazine]:
2′-(2-(methylsulfonyl)pyrimidin-5-yl)-6′,8′-dihydrospiro[chroman-4,9′-pyrido[3′,2′:4,5]imidazo[2,1-c][1,4]oxazine];
2′-(2-(1,4-diazepan-1-yl)pyrimidin-5-yl)-6′,8′-dihydrospiro[chroman-4,9′-pyrido[3′,2′:4,5]imidazo[2,1-c][1,4]oxazine];
5-(6′,8′-dihydrospiro[chroman-4,9′-pyrido[3′,2′:4,5]imidazo[2,1-c][1,4]oxazin]-2′-yl)-N-isopropylpyrimidin-2-amine;
2′-(2-morpholinopyrimidin-5-yl)-6′,8-dihydrospiro[chroman-4,9′-pyrido[3′,2′:4,5]imidazo[2,1-c][1,4]oxazine];
2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol;
2′-(2-((tetrahydro-2H-pyran-4-yl)oxy)pyridin-4-yl)-6′,8′-dihydrospiro[chroman-4,9′-pyrido[3′,2′:4,5]imidazo[2,1-c][1,4]oxazine];
5-(6′H,8′H-Spiro[chromane-4,9′-pyrido[3′,2′:4,5]imidazo[2,1-c][1,4]oxazin]-2′-yl)-N-(tetrahydrofuran-3-yl)pyrimidin-2-amine;
(8aR)-7-(5-(6′,8′-dihydrospiro[chroman-4,9′-pyrido[3′,2′:4,5]imidazo[2,1-c][1,4]oxazin]-2′-yl)pyrimidin-2-yl)hexahydroimidazo[1,5-a]pyrazin-3(2H)-one;
1-(5-(6′,8′-dihydrospiro[chroman-4,9′-pyrido[3′,2′:4,5]imidazo[2,1-c][1,4]oxazin]-2′-yl)pyrimidin-2-yl)piperidin-4-ol;
1-(5-(6′,8′-dihydrospiro[chroman-4,9′-pyrido[3′,2′:4,5]imidazo[2,1-c][1,4]oxazin]-2′-yl)pyrimidin-2-yl)piperidin-3-ol;
1-(5-(6′,8′-dihydrospiro[chroman-4,9′-pyrido[3′,2′:4,5]imidazo[2,1-c][1,4]oxazin]-2′-yl)pyrimidin-2-yl)azetidin-3-ol;
1-(5-(6′,8′-dihydrospiro[chroman-4,9′-pyrido[3′,2′:4,5]imidazo[2,1-c][1,4]oxazin]-2′-yl)pyrimidin-2-yl)pyrrolidin-3-ol;
3-((5-(6′,8′-dihydrospiro[chroman-4,9′-pyrido[3′,2′:4,5]imidazo[2,1-c][1,4]oxazin]-2′-yl)pyrimidin-2-yl)amino)cyclobutanol;
1-(5-(6′,8′-dihydrospiro[chroman-4,9′-pyrido[3′,2′:4,5]imidazo[2,1-c][1,4]oxazin]-2′-yl)pyrimidin-2-yl)-3-methylazetidin-3-ol;
2′-(2-(4-methylpiperazin-1-yl)pyrimidin-5-yl)-6′,8′-dihydrospiro[chroman-4,9′-pyrido[3′,2′:4,5]imidazo[2,1-c][1,4]oxazine];
2′-(5,5-Dimethyl-2,5-dihydro-1H-pyrrol-3-yl)-2H,6′H,8′H-spiro[benzofuran-3,9′-pyrido[3′,2′:4,5]imidazo[2,1-c][1,4]oxazine];
2′-(2,5-dihydro-1H-pyrrol-3-yl)-6′,8′-dihydro-2H-spiro[benzofuran-3,9′-pyrido[3′,2′:4,5]imidazo[2,1-c][1,4]oxazine];
2′-(2-(piperazin-1-yl)pyrimidin-5-yl)-2H,6′H,8′H-spiro[benzofuran-3,9′-pyrido[3′,2′:4,5]imidazo[2,1-c][1,4]oxazine];
2′-(2-((tetrahydro-2H-pyran-4-yl)oxy)pyridin-4-yl)-6′,8′-dihydro-2H-spiro[benzofuran-3,9′-pyrido[3′,2′:4,5]imidazo[2,1-c][1,4]oxazine];
2′-(2-methoxypyrimidin-5-yl)-6′,8′-dihydro-2H-spiro[benzofuran-3,9′-pyrido[3′,2′:4,5]imidazo[2,1-c][1,4]oxazine];
2-(tert-butoxy)-1-((2S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)ethanone;
2-(tert-butoxy)-1-((3S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)ethanone;
2-(tert-butoxy)-1-((3R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)ethanone;
2-(tert-butoxy)-1-((2R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)ethanone;
1-((2S)-4-(5-(2H,6′H,8′H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethan-1-one
1-((3S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)-2-hydroxyethanone;
1-((3R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)-2-hydroxyethanone;
1-((2R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone;
2-(5-(2,3-dihydro-6′H,8′H-spiro[indene-1,9′-pyrido[3′,2′:4,5]imidazo[2,1-c][1,4]oxazin]-2′-yl)pyrimidin-2-yl)propan-2-ol;
2′-(2-(2-hydroxypropan-2-yl)pyrimidin-5-yl)-6′,8′-dihydrospiro[indene-1,9′-pyrido[3′,2′:4,5]imidazo[2,1-c][1,4]oxazin]-3(2H)-one;
2′-(2-(2-hydroxypropan-2-yl)pyrimidin-5-yl)-2,3-dihydro-6′H,8′H-spiro[indene-1,9′-pyrido[3′,2′:4,5]imidazo[2,1-c][1,4]oxazin]-6′-ol;
2′-(1-(pyrimidin-4-yl)-1,2,3,6-tetrahydropyridin-4-yl)-2H,6′H,8′H-spiro[benzofuran-3,9′-pyrido[3′,2′:4,5]imidazo[2,1-c][1,4]oxazine];
1-(4-(6′,8′-dihydro-2H-spiro[benzofuran-3,9′-pyrido[3′,2′:4,5]imidazo[2,1-c][1,4]oxazin]-2′-yl)-5,6-dihydropyridin-1(2H)-yl)-3-methoxy-3-methylbutan-1-one;
(S)-1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-4-ol;
(R)-7-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3(2H)-one;
(1S,4r)-4-((4-((S)-6′,8′-Dihydro-2H-spiro[benzofuran-3,9′-pyrido[3′,2′:4,5]imidazo[2,1-c][1,4]oxazin]-2′-yl)pyridin-2-yl)oxy)cyclohexanol;
1-((S)-4-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone;
1-((R)-4-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone;
1-((R)-4-(5-((R)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone;
1-((S)-4-(5-((R)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone;
1-(5-(6′,8′-dihydrospiro[isochroman-4,9′-pyrido[3′,2′:4,5]imidazo[2,1-c][1,4]oxazin]-2′-yl)pyrimidin-2-yl)piperidin-4-ol;
(8aR)-7-(5-(6′,8′-dihydrospiro[isochroman-4,9′-pyrido[3′,2′:4,5]imidazo[2,1-c][1,4]oxazin]-2′-yl)pyrimidin-2-yl)hexahydroimidazo[1,5-a]pyrazin-3(2H)-one;
2′-(2-morpholinopyrimidin-5-yl)-6′,8′-dihydrospiro[isochroman-4,9′-pyrido[3′,2′:4,5]imidazo[2,1-c][1,4]oxazine];
2-(5-(2,3-dihydro-6′H,8′H-spiro[indene-1,9′-pyrido[3′,2′:4,5]imidazo[2,1-c][1,4]oxazin]-2′-yl)pyrimidin-2-yl)propan-2-amine;
(S)-2-(5-(6′,8′-dihydro-2H-spiro[benzofuran-3,9′-pyrido[3′,2′:4,5]imidazo[2,1-c][1,4]oxazin]-2′-yl)pyrimidin-2-yl)propan-2-amine; or
2′-(2-(2-hydroxypropan-2-yl)pyrimidin-5-yl)-2,3,6′,8′-tetrahydrospiro[indene-1,9′-pyrido[3′,2′:4,5]imidazo[2,1-c][1,4]oxazin]-3-ol.

In certain embodiments, the compound is selected from the group consisting of:
((R)-1-(5-((R)-8-phenyl-7,8-dihydro-6H-pyrrolo[2′,1′:2,3]imidazo[4,5-b]pyridin-2-yl)pyrimidin-2-yl)pyrrolidin-2-yl)methanol;
((S)-1-(5-((R)-8-phenyl-7,8-dihydro-6H-pyrrolo[2′,1′:2,3]imidazo[4,5-b]pyridin-2-yl)pyrimidin-2-yl)pyrrolidin-2-yl)methanol;
(R)-1-(2-(methylsulfonyl)ethyl)-4-(8-phenyl-7,8-dihydro-6H-pyrrolo[2′,1′:2,3]imidazo[4,5-b]pyridin-2-yl)pyridin-2(1H)-one;
(R)-1-(2-hydroxy-2-methylpropyl)-4-(8-phenyl-7,8-dihydro-6H-pyrrolo[2′,1′:2,3]imidazo[4,5-b]pyridin-2-yl)pyridin-2(1H)-one;
1-(5-((R)-8-phenyl-7,8-dihydro-6H-pyrrolo[2′,1′:2,3]imidazo[4,5-b]pyridin-2-yl)pyrimidin-2-yl)ethanol;
2-cyclopropyl-1-(5-((R)-8-phenyl-7,8-dihydro-6H-pyrrolo[2′,1′:2,3]imidazo[4,5-b]pyridin-2-yl)pyrimidin-2-yl)ethanol;
(5-((R)-8-phenyl-7,8-dihydro-6H-pyrrolo[2′,1′:2,3]imidazo[4,5-b]pyridin-2-yl)pyrimidin-2-yl)(tetrahydro-2H-pyran-4-yl)methanol;
1-(5-((R)-8-phenyl-7,8-dihydro-6H-pyrrolo[2′,1′:2,3]imidazo[4,5-b]pyridin-2-yl)pyrimidin-2-yl)-1-(tetrahydro-2H-pyran-4-yl)ethanol;
1-cyclopropyl-2-(5-((R)-8-phenyl-7,8-dihydro-6H-pyrrolo[2′,1′:2,3]imidazo[4,5-b]pyridin-2-yl)pyrimidin-2-yl)propan-2-ol;
1-((R)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone;
1-((S)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone;
(1R,3R)-3-((4-((R)-8-phenyl-7,8-dihydro-6H-pyrido[3,2-b]pyrrolizin-2-yl)pyridin-2-yl)oxy)cyclopentanecarbonitrile;
(R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2(1H)-one;
2-(5-(8-(pyridin-2-yl)-7,8-dihydro-6H-pyrrolo[2′,1′:2,3]imidazo[4,5-b]pyridin-2-yl)pyrimidin-2-yl)propan-2-ol;
1-((S)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone;
2-hydroxy-1-((R)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone;
(R)-7-(5-((R)-8-(3-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
1-((R)-4-(5-((R)-8-(3-methoxyphenyl)-7,8-dihydro-6H-pyrrolo[2′,1′:2,3]imidazo[4,5-b]pyridin-2-yl)pyrimidin-2-yl)-2-methylpiperazin-1-yl)ethanone;
(R)-7-(5-((S)-8-(3-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
(R)-7-(5-((R)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
(S)-7-(5-((R)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
(R)-1-(5-(8-(2-methoxyphenyl)-7,8-dihydro-6H-pyrrolo[2′,1′:2,3]imidazo[4,5-b]pyridin-2-yl)pyrimidin-2-yl)piperidin-4-ol;
2-hydroxy-1-((R)-4-(5-((R)-8-(2-methoxyphenyl)-7,8-dihydro-6H-pyrrolo[2′,1′:2,3]imidazo[4,5-b]pyridin-2-yl)pyrimidin-2-yl)-2-methylpiperazin-1-yl)ethanone;
(R)-7-(5-((S)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one;
1-((R)-4-(5-((R)-8-(3-(Hydroxymethyl)phenyl)-7,8-dihydro-6H-pyrrolo[2′,1′:2,3]imidazo[4,5-b]pyridin-2-yl)pyrimidin-2-yl)-2-methylpiperazin-1-yl)ethanone;
1-((R)-4-(5-((R)-8-(3-(Hydroxymethyl)phenyl)-7,8-dihydro-6H-pyrrolo[2′,1′:2,3]imidazo[4,5-b]pyridin-2-yl)pyrimidin-2-yl)-2-methylpiperazin-1-yl) ethanone;
(S)-1-(5-((R)-8-phenyl-7,8-dihydro-6H-pyrrolo[2′,1′:2,3]imidazo[4,5-b]pyridin-2-yl)pyrimidin-2-yl)piperidin-4-yl 2-amino-3-methylbutanoate;
(R)-1-(5-(8-phenyl-7,8-dihydro-6H-pyrrolo[2′,1′:2,3]imidazo[4,5-b]pyridin-2-yl)pyrimidin-2-yl)piperidin-4-yl dihydrogen phosphate hydrochloride;
(R)-8-phenyl-2-(2-((tetrahydro-2H-pyran-4-yl)oxy)pyridin-4-yl)-7,8-dihydro-6H-pyrrolo[2′,1′:2,3]imidazo[4,5-b]pyridine;
3-((4-((R)-8-phenyl-7,8-dihydro-6H-pyrrolo[2′,1:2,3]imidazo[4,5-b]pyridin-2-yl)pyridin-2-yl)oxy)cyclopentanol;
(R)-4-((4-(8-phenyl-7,8-dihydro-6H-pyrrolo[2′,1:2,3]imidazo[4,5-b]pyridin-2-yl)pyridin-2-yl)oxy)cyclohexanol;
(R)-2-(2-(oxetan-3-yloxy)pyridin-4-yl)-8-phenyl-7,8-dihydro-6H-pyrrolo[2′,1′:2,3]imidazo[4,5-b]pyridine;
3-((4-((R)-8-phenyl-7,8-dihydro-6H-pyrrolo[2′,1:2,3]imidazo[4,5-b]pyridin-2-yl)pyridin-2-yl)oxy)cyclohexanol;
(R)-2-(2-(oxetan-3-ylmethoxy)pyridin-4-yl)-8-phenyl-7,8-dihydro-6H-pyrrolo[2′,1′:2,3]imidazo[4,5-b]pyridine;
(R)-2-(2-(((R)-1-methylpyrrolidin-3-yl)oxy)pyridin-4-yl)-8-phenyl-7,8-dihydro-6H-pyrrolo[2′,1′:2,3]imidazo[4,5-b]pyridine;
(R)-2-((4-(8-phenyl-7,8-dihydro-6H-pyrrolo[2′,1:2,3]imidazo[4,5-b]pyridin-2-yl)pyridin-2-yl)oxy)ethanol;
(R)-2-(2-(((S)-1-methylpyrrolidin-3-yl)oxy)pyridin-4-yl)-8-phenyl-7,8-dihydro-6H-pyrrolo[2′,1′:2,3]imidazo[4,5-b]pyridine;
(1S,4s)-4-((4-((R)-8-phenyl-7,8-dihydro-6H-pyrrolo[2′,1′:2,3]imidazo[4,5-b]pyridin-2-yl)pyridin-2-yl)oxy)cyclohexanol;
(8R)-8-phenyl-2-(2-((tetrahydro-2H-pyran-3-yl)oxy)pyridin-4-yl)-7,8-dihydro-6H-pyrrolo[2′,1′:2,3]imidazo[4,5-b]pyridine;
(8R)-8-phenyl-2-(2-((tetrahydrofuran-3-yl)oxy)pyridin-4-yl)-7,8-dihydro-6H-pyrrolo[2′,1′:2,3]imidazo[4,5-b]pyridine;
(R)-2-(2-(cyclopentyloxy)pyridin-4-yl)-8-phenyl-7,8-dihydro-6H-pyrrolo[2′,1′:2,3]imidazo[4,5-b]pyridine;
(R)-2-(2-(cyclohexyloxy)pyridin-4-yl)-8-phenyl-7,8-dihydro-6H-pyrrolo[2′,1′:2,3]imidazo[4,5-b]pyridine;
(R)-methyl 4-((4-(8-phenyl-7,8-dihydro-6H-pyrrolo[2′,1′:2,3]imidazo[4,5-b]pyridin-2-yl)pyridin-2-yl)oxy)cyclohexanecarboxylate;
methyl 3-((4-((R)-8-phenyl-7,8-dihydro-6H-pyrrolo[2′,1′:2,3]imidazo[4,5-b]pyridin-2-yl)pyridin-2-yl)oxy)cyclopentanecarboxylate;
(R)-2-(2-butoxypyridin-4-yl)-8-phenyl-7,8-dihydro-6H-pyrido[3,2-b]pyrrolizine;
(1R,3R)-3-((4-((R)-8-phenyl-7,8-dihydro-6H-pyrido[3,2-b]pyrrolizin-2-yl)pyridin-2-yl)oxy)cyclopentanecarbonitrile;
(R)-8-phenyl-2-(2-((S)-pyrrolidin-3-yloxy)pyridin-4-yl)-7,8-dihydro-6H-pyrrolo[2′,1′:2,3]imidazo[4,5-b]pyridine;
(8R)-8-phenyl-2-(2-(piperidin-3-yloxy)pyridin-4-yl)-7,8-dihydro-6H-pyrrolo[2′,1′:2,3]imidazo[4,5-b]pyridine;
(R)-8-phenyl-2-(2-((R)-pyrrolidin-3-yloxy)pyridin-4-yl)-7,8-dihydro-6H-pyrrolo[2′,1′:2,3]imidazo[4,5-b]pyridine;
(8R)-2-(2-(6-azaspiro[3.4]octan-1-yloxy)pyridin-4-yl)-8-phenyl-7,8-dihydro-6H-pyrrolo[2′,1′:2,3]imidazo[4,5-b]pyridine;
(R)-2-(2-(6-azaspiro[3.4]octan-2-yloxy)pyridin-4-yl)-8-phenyl-7,8-dihydro-6H-pyrrolo[2′,1′:2,3]imidazo[4,5-b]pyridine;
(8R)-2-(2-(6-azaspiro[3.5]nonan-1-yloxy)pyridin-4-yl)-8-phenyl-7,8-dihydro-6H-pyrrolo[2′,1′:2,3]imidazo[4,5-b]pyridine;
1-(5-(6′,8′-sihydro-2H-spiro[benzofuran-3,9′-pyrido[3′,2′:4,5]imidazo[2,1-c][1,4]oxazin]-2′-yl)pyrimidin-2-yl)piperidin-4-ol;
(R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2(1H)-one;
(R)-1-(2-methoxyethyl)-4-(8-phenyl-7,8-dihydro-6H-pyrrolo[2′,1′:2,3]imidazo[4,5-b]pyridin-2-yl)pyridin-2(1H)-one;
(R)-4-(8-phenyl-7,8-dihydro-6H-pyrrolo[2′,1′:2,3]imidazo[4,5-b]pyridin-2-yl)-1-((tetrahydro-2H-pyran-4-yl)methyl)pyridin-2(1H)-one;
4-((R)-8-phenyl-7,8-dihydro-6H-pyrrolo[2′,1′:2,3]imidazo[4,5-b]pyridin-2-yl)-1-(tetrahydrofuran-3-yl)pyridin-2(1H)-one;
(R)-4-(8-phenyl-7,8-dihydro-6H-pyrrolo[2′,1′:2,3]imidazo[4,5-b]pyridin-2-yl)-1-(tetrahydro-2H-pyran-4-yl)pyridin-2(1H)-one;
(R)-8-phenyl-2-(1-(tetrahydro-2H-pyran-4-yl)-1H-pyrazol-4-yl)-7,8-dihydro-6H-pyrrolo[2′,1′:2,3]imidazo[4,5-b]pyridine;
4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol;
(R)-1-(5-(3-fluoro-8-phenyl-7,8-dihydro-6H-pyrrolo[2′,1′:2,3]imidazo[4,5-b]pyridin-2-yl)pyrimidin-2-yl)piperidin-4-ol;
2-(2-morpholinopyrimidin-5-yl)-9-phenyl-7,9-dihydro-6H-pyran[4′,3′:4,5]imidazo[1,2-b]pyridazine;
trans-4-((4-(4-(2-methoxyphenyl)-3,4-dihydro-2H-pyran[2′,3′:4,5]imidazo[1,2-a]pyridin-7-yl)pyridin-2-yl)oxy)cyclohexanol;
(8aS)-7-(5-(9-phenyl-8,9-dihydro-6H-pyrido[3′,2′:4,5]imidazo[2,1-c][1,4]oxazin-2-yl)pyrimidin-2-yl)hexahydroimidazo[1,5-a]pyrazin-3(2H)-one;
3,3-difluoro-1-(5-(9-phenyl-8,9-dihydro-6H-pyrido[3′,2′:4,5]imidazo[2,1-c][1,4]oxazin-2-yl)pyrimidin-2-yl)piperidin-4-ol;
1-(5-(9-phenyl-8,9-dihydro-6H-pyrido[3′,2′:4,5]imidazo[2,1-c][1,4]oxazin-2-yl)pyrimidin-2-yl)piperidin-4-ol;
2-(2-(1,4-oxazepan-4-yl)pyrimidin-5-yl)-9-phenyl-8,9-dihydro-6H-pyrido[3′,2′:4,5]imidazo[2,1-c][1,4]oxazine;
(1-(5-(9-phenyl-8,9-dihydro-6H-pyrido[3′,2′:4,5]imidazo[2,1-c][1,4]oxazin-2-yl)pyrimidin-2-yl)piperidin-3-yl)methanol;
(4-fluoro-1-(5-(9-phenyl-8,9-dihydro-6H-pyrido[3′,2′:4,5]imidazo[2,1-c][1,4]oxazin-2-yl)pyrimidin-2-yl)piperidin-4-yl)methanol;
2-hydroxy-1-(4-(5-(9-phenyl-8,9-dihydro-6H-pyrido[3′,2′:4,5]imidazo[2,1-c][1,4]oxazin-2-yl)pyrimidin-2-yl)piperazin-1-yl)propan-1-one;
2-hydroxy-1-(4-(5-(9-phenyl-8,9-dihydro-6H-pyrido[3′,2′:4,5]imidazo[2,1-c][1,4]oxazin-2-yl)pyrimidin-2-yl)piperazin-1-yl)ethanone;
(R)-8-phenyl-2-(2-((tetrahydro-2H-pyran-4-yl))methoxy)pyridin-4-yl)-7,8-dihydro-6H-pyrrolo[2′,1′:2,3]imidazo[4,5-b]pyridine;
(R)-2-(2-(2-methoxyethoxy)pyridin-4-yl)-8-phenyl-7,8-dihydro-6H-pyrrolo[2′,1′:2,3]imidazo[4,5-b]pyridine;
(R)-2-methyl-1-((4-(8-phenyl-7,8-dihydro-6H-pyrrolo[2′,1′:2,3]imidazo[4,5-b]pyridin-2-yl)pyridin-2-yl)oxy)propan-2-ol;
(R)-8-phenyl-2-(1,2,3,6-tetrahydropyridin-4-yl)-7,8-dihydro-6H-pyrrolo[2′,1′:2,3]imidazo[4,5-b]pyridine;
(1S,4s)-4-(((4-((R)-8-phenyl-7,8-dihydro-6H-pyrrolo[2′,1′:2,3]imidazo[4,5-b]pyridin-2-yl)pyridin-2-yl)oxy)methyl)cyclohexanol;
(1R,4r)-4-(((4-((R)-8-phenyl-7,8-dihydro-6H-pyrrolo[1′:22′,1:2,3]imidazo[4,5-b]pyridin-2-yl)pyridin-2-yl)oxy)methyl)cyclohexanol;
((1R,4r)-4-(((4-((R)-8-phenyl-7,8-dihydro-6H-pyrrolo[2′,1′:2,3]imidazo[4,5-b]pyridin-2-yl)pyridin-2-yl)oxy)methyl)cyclohexyl)methanol;
(R)-8-Phenyl-2-(1-(pyrimidin-4-yl)-1,2,3,6-tetrahydropyridin-4-yl)-7,8-dihydro-6H-pyrrolo[2′,1′:2,3]imidazo[4,5-b]pyridine;
methyl 2-(4-((R)-8-phenyl-7,8-dihydro-6H-pyrrolo[2′,1′:2,3]imidazo[4,5-b]pyridin-2-yl)cyclohex-3-en-1-yl)acetate;
1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol;
(S)-1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol;
(R)-1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol;
(S)-2-(5-(8-methyl-8-phenyl-7,8-dihydro-6H-pyrrolo[2′,1′:2,3]imidazo[4,5-b]pyridin-2-yl)pyrimidin-2-yl)propan-2-ol;
(R)-2-(5-(8-methyl-8-phenyl-7,8-dihydro-6H-pyrrolo[2′,1′:2,3]imidazo[4,5-b]pyridin-2-yl)pyrimidin-2-yl)propan-2-ol;
(R)-2-(5-(8-phenyl-7,8-dihydro-6H-pyrrolo[2′,1′:2,3]imidazo[4,5-b]pyridin-2-yl)pyrimidin-2-yl)propan-2-amine;
2-(2-(4,4-difluoropiperidin-1-yl)pyrimidin-5-yl)-9-phenyl-8,9-dihydro-6H-pyrido-[3′,2′:4,5]imidazo[2,1-c][1,4]oxazine; or
(1R,4R)-4-((4-((R)-8-phenyl-7,8-dihydro-6H-pyrrolo[2′,1′:2,3]imidazo[4,5-b]pyridin-2-yl)pyridin-2-yl)oxy)cyclohexanol.
In certain embodiments, the compound is selected from the group consisting of:
2-(5-(9-phenyl-6,7,8,9-tetrahydro-6,8-methanoimidazo[1,2-a:5,4-b′]dipyridin-2-yl)pyrimidin-2-yl)propan-2-ol;
9-phenyl-2-(2-((tetrahydro-2H-pyran-4-yl)oxy)pyridin-4-yl)-6,7,8,9-tetrahydro-6,8-methanoimidazo[1,2-a:5,4-b′]dipyridine;
4-(5-(9-phenyl-6,7,8,9-tetrahydro-6,8-methanoimidazo[1,2-a:5,4-b′]dipyridin-2-yl)pyrimidin-2-yl)morpholine;
1-(5-(9-phenyl-6,7,8,9-tetrahydro-6,8-methanoimidazo[1,2-a:5,4-b′]dipyridin-2-yl)pyrimidin-2-yl)piperidin-4-ol;
2-(5-((6R,8S,9S)-9-phenyl-6,7,8,9-tetrahydro-6,8-epoxyimidazo[1,2-a:5,4-b′]dipyridin-2-yl)pyrimidin-2-yl)propan-2-ol;
1-(5-((6R,8S,9S)-9-phenyl-6,7,8,9-tetrahydro-6,8-epoxyimidazo[1,2-a:5,4-b′]dipyridin-2-yl)pyrimidin-2-yl)piperidin-4-ol;
2-(5-(9-phenyl-6,7,8,9-tetrahydro-6,8-methanoimidazo[1,2-a:5,4-b′]dipyridin-2-yl)pyrimidin-2-yl)propan-2-ol;
9-phenyl-2-(2-((tetrahydro-2H-pyran-4-yl)oxy)pyridin-4-yl)-6,7,8,9-tetrahydro-6,8-methanoimidazo[1,2-a:5,4-b′]dipyridine;
1-(5-(9-phenyl-6,7,8,9-tetrahydro-6,8-methanoimidazo[1,2-a:5,4-b′]dipyridin-2-yl)pyrimidin-2-yl)piperidin-4-ol;
4-(5-(9-phenyl-6,7,8,9-tetrahydro-6,8-methanoimidazo[1,2-a:5,4-b′]dipyridin-2-yl)pyrimidin-2-yl)morpholine;
2-(5-((6R,8S,9S)-9-phenyl-6,7,8,9-tetrahydro-6,8-epoxyimidazo[1,2-a:5,4-b′]dipyridin-2-yl)pyrimidin-2-yl)propan-2-ol; or
1-(5-((6R,8S,9S)-9-phenyl-6,7,8,9-tetrahydro-6,8-epoxyimidazo[1,2-a:5,4-b′]dipyridin-2-yl)pyrimidin-2-yl)piperidin-4-ol.

The disclosures of U.S. Pat. Nos. 10,266,532 B2 and 9,856,253 B2, and U.S. Patent Applications No. US20160304517A1 and US2018/0179198 A1, are incorporated herein in their entireties by reference.

In certain embodiments, the TNF binder comprises a compound of formula (2c):

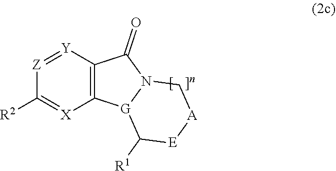

or a pharmaceutically acceptable salt, tautomer, geometric isomer, or stereoisomer thereof,
wherein:
X, Y, and Z are independently CR⁴ or N;
   provided that Y and Z are not both N;
A is —C(R^{z})₂—;
E is CH₂ or O and G is CH; or E is CH₂ and G is CH or N;
R¹ is optionally substituted aryl or optionally substituted heteroaryl;
R² is —R^{2a}—R^{2b}, wherein:
   R^{2a} is an optionally substituted saturated, unsaturated or partially saturated heterocyclyl or optionally substituted heteroaryl;
   R^{2b} is —N(R^{a}(R^{b}), —O(R^{a}), optionally substituted (C₁-C₅)alkyl, optionally substituted (C₃-C₆)cycloalkyl, —(CH₂)ₚ-optionally substituted heteroaryl or —(CH₂)ₚ-optionally substituted heterocyclyl; wherein
      R^{a} and R^{b} are independently selected from H, optionally substituted (C₁-C₅)alkyl, and —(CH₂)ₙ-optionally substituted heterocyclyl;
R⁴ is independently H, halo, CF₃, or (C₁-C₃)alkyl;
R^{z} is independently H, halo, CF₃, or (C₁-C₃)alkyl;
n is 0 or 1; and
p is 0 or 1.

In certain embodiments, the compound of formula (2c) comprises

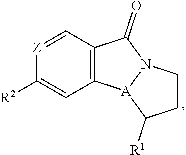

wherein G is N or CH; and Z is CH or CF.

In certain embodiments of the compound of formula (2c), R¹ is selected from the group consisting of:

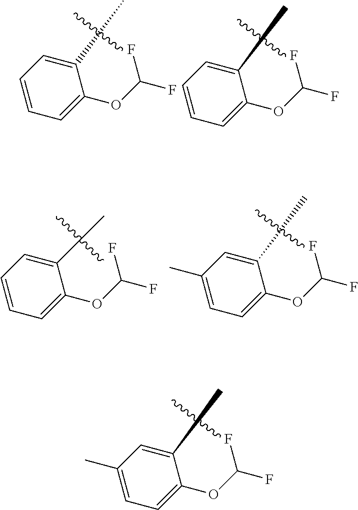

In certain embodiments of the compound of formula (2c), R² is selected from the group consisting of:

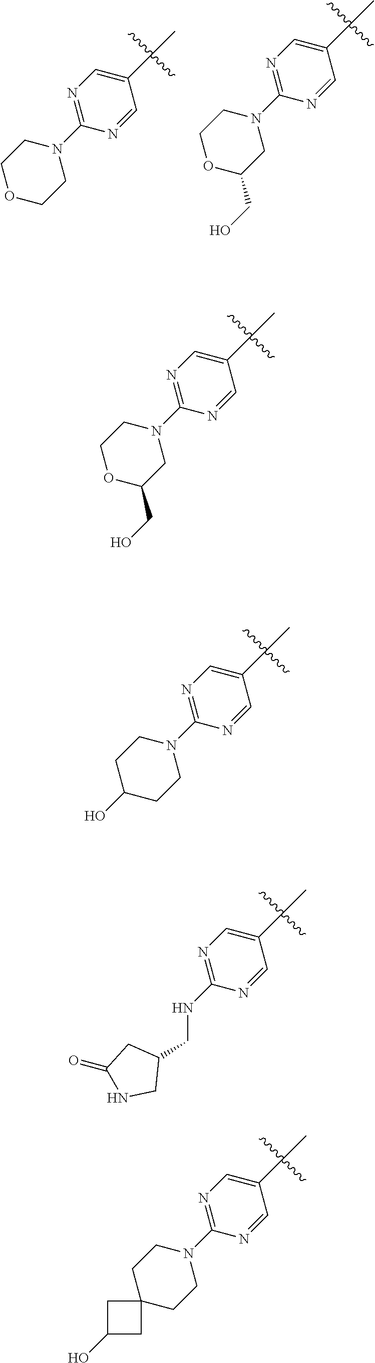

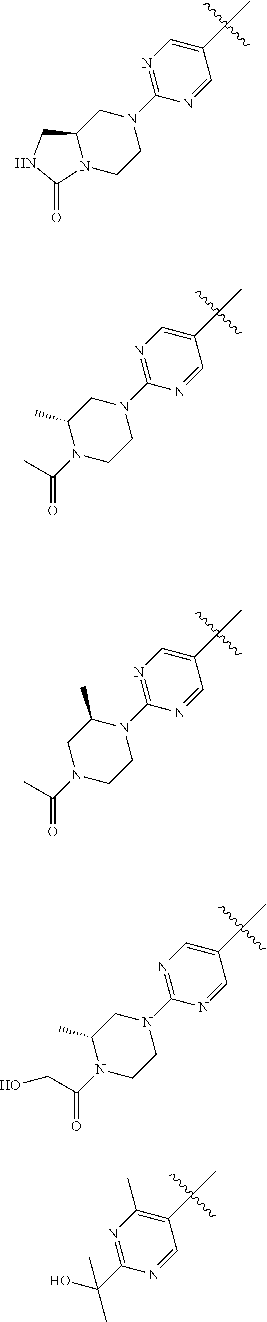 [SMILES: *c1cnc(N2CCN3C(=O)NC[C@H]3C2)nc1 ; *c1cnc(N2CCN(C(C)=O)[C@H](C)C2)nc1 ; *c1cnc(N2CCN(C(C)=O)C[C@H]2C)nc1 ; *c1cnc(N2CCN(C(=O)CO)[C@H](C)C2)nc1 ; *c1cnc(C(C)(C)O)nc1C]

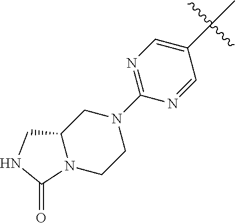 [SMILES: *c1cnc(N2CCN3C(=O)NC[C@@H]3C2)nc1]

In certain embodiments, R² can be used to attach the TNF linker to the compound of the disclosure. This can be done, for example, using any hydroxyl, amino, amido, thiyl, or carboxylic acid group that is present in R² as listed herein or that can be introduced therein. In any such cases, the hydroxyl group in R² can be used for example to form an ester bond; the carboxylic group in R² can be used for example to form an ester bond or an amide bond; the amino group in R² can be used for example to form an amide group and an imine group, and so forth; the amino, amido, or thiyl group in R² can be used for example to form a chemical linkage through alkylation or nucleophilic displacement, and so forth, as known to those skilled in the art.

In certain embodiments, the compound is selected from the group consisting of:
3-(2-(Difluoromethoxy)phenyl)-6-(2-morpholinopyrimidin-5-yl)-2,3-dihydropyrazolo[1,2-a]indazol-9 (1H)-one;
(R)-3-(2-(difluoromethoxy)phenyl)-6-(2-morpholinopyrimidin-5-yl)-2,3-dihydropyrazolo[1,2-a]indazol-9(1H)-one;
(S)-3-(2-(difluoromethoxy)phenyl)-6-(2-morpholinopyrimidin-5-yl)-2,3-dihydropyrazolo[1,2-a]indazol-9(1H)-one;
1-(5-(3-(2-(difluoromethoxy)phenyl)-9-oxo-1,2,3,9-tetrahydropyrazolo[1,2-a]indazol-6-yl)pyrimidin-2-yl)piperidine-4-carboxylic acid;
(S)-3-(2-(difluoromethoxy)phenyl)-6-(2-((R)-2-(methoxymethyl)pyrrolidin-1-yl)pyrimidin-5-yl)-2,3-dihydropyrazolo[1,2-a]indazol-9(1H)-one;
(R)-3-(2-(difluoromethoxy)phenyl)-6-(2-((R)-2-(methoxymethyl)pyrrolidin-1-yl)pyrimidin-5-yl)-2,3-dihydropyrazolo[1,2-a]indazol-9(1H)-one;
(R)-3-(2-(difluoromethoxy)phenyl)-6-(2-(((R)-tetrahydrofuran-3-yl)amino)pyrimidin-5-yl)-2,3-dihydropyrazolo[1,2-a]indazol-9(1H)-one;
(S)-3-(2-(difluoromethoxy)phenyl)-6-(2-(((R)-tetrahydrofuran-3-yl)amino)pyrimidin-5-yl)-2,3-dihydropyrazolo[1,2-a]indazol-9(1H)-one;
(R)-3-(2-(difluoromethoxy)phenyl)-6-(2-((R)-2-(hydroxymethyl)morpholino)pyrimidin-5-yl)-2,3-dihydropyrazolo[1,2-a]indazol-9(1H)-one;
(S)-3-(2-(difluoromethoxy)phenyl)-6-(2-((R)-2-(hydroxymethyl)morpholino)pyrimidin-5-yl)-2,3-dihydropyrazolo[1,2-a]indazol-9(1H)-one;
(S)-3(2-(difluoromethoxy)phenyl)-6-(2-((S)-2-(hydroxymethyl)morpholino)pyrimidin-5-yl)-2,3-dihydropyrazolo[1,2-a]indazol-9(1H)-one;
(R)-3-(2-(difluoromethoxy)phenyl)-6-(2-((S)-2-(hydroxymethyl)morpholino)pyrimidin-5-yl)-2,3-dihydropyrazolo[1,2-a]indazol-9(1H)-one;
(R)-3-(2-(Difluoromethoxy)phenyl)-6-(2-(2-hydroxypropan-2-yl)pyrimidin-5-yl)-2,3-dihydropyrazolo[1,2-a]indazol-9(1H)-one;
(S)-3-(2-(difluoromethoxy)phenyl)-6-(2-(2-hydroxypropan-2-yl)pyrimidin-5-yl)-2,3-dihydropyrazolo[1,2-a]indazol-9(1H)-one;
3-(2-(difluoromethoxy)phenyl)-6-(2-(4-hydroxypiperidin-1-yl)pyrimidin-5-yl)-2,3-dihydropyrazolo[1,2-a]indazol-9(1H)-one;
(R)-3-(2-(difluoromethoxy)phenyl)-6-(2-((((S)-5-oxopyrrolidin-3-yl)methyl)amino)pyrimidin-5-yl)-2,3-dihydropyrazolo[1,2-a]indazol-9(1H)-one;
(S)-3-(2-(difluoromethoxy)phenyl)-6-(2-((((S)-5-oxopyrrolidin-3-yl)methyl)amino)pyrimidin-5-yl)-2,3-dihydropyrazolo[1,2-a]indazol-9(1H)-one;
(R)-3-(2-(difluoromethoxy)phenyl)-6-(2-((((R)-5-oxopyrrolidin-3-yl)methyl)amino)pyrimidin-5-yl)-2,3-dihydropyrazolo[1,2-a]indazol-9(1H)-one;
(S)-3-(2-(difluoromethoxy)phenyl)-6-(2-((((R)-5-oxopyrrolidin-3-yl)methyl)amino)pyrimidin-5-yl)-2,3-dihydropyrazolo[1,2-a]indazol-9(1H)-one;
(R)-3-(2-(difluoromethoxy)phenyl)-6-(2-(2-hydroxy-7-azaspiro[3.5]nonan-7-yl)pyrimidin-5-yl)-2,3-dihydropyrazolo[1,2-a]indazol-9(1H)-one;
(S)-3-(2-(difluoromethoxy)phenyl)-6-(2-(2-hydroxy-7-azaspiro[3.5]nonan-7-yl)pyrimidin-5-yl)-2,3-dihydropyrazolo[1,2-a]indazol-9(1H)-one;
(S)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one;
(S)-3-(2-(difluoromethoxy)phenyl)-6-(2-((R)-3-oxohexahydroimidazo[1,5-a]pyrazin-7(1H)-yl)pyrimidin-5-yl)-2,3-dihydropyrazolo[1,2-a]indazol-9(1H)-one;
(R)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one;
(R)-3-(2-(difluoromethoxy)phenyl)-6-(2-((R)-3-oxohexahydroimidazo[1,5-a]pyrazin-7(1H)-yl)pyrimidin-5-yl)-2,3-dihydropyrazolo[1,2-a]indazol-9(1H)-one;
(R)-3-(2-(Difluoromethoxy)phenyl)-6-(2-((3--methoxypropyl)amino)pyrimidin-5-yl)-2,3-dihydropyrazolo[1,2-a]indazol-9(1H)-one;
(S)-3-(2-(Difluoromethoxy)phenyl)-6-(2-((3-methoxypropyl)amino)pyrimidin-5-yl)-2,3-dihydropyrazolo[1,2-a]indazol-9(1H)-one;
(S)-6-(2-(4-Acetylpiperazin-1-yl)pyrimidin-5-yl)-3-(2-(difluoromethoxy)phenyl)-2,3-dihydropyrazolo[1,2-a]indazol-9(1H)-one;
(R)-6-(2-(4-Acetylpiperazin-1-yl)pyrimidin-5-yl)-3-(2-(difluoromethoxy)phenyl)-2,3-dihydropyrazolo[1,2-a]indazol-9(1H)-one;
6-(2-(difluoromethoxy)phenyl)-3-(2-morpholinopyrimidin-5-yl)-8,9-dihydro-6H-pyridazino[1,2-a]indazol-11(7H)-one;
6-(2-(difluoromethoxy)phenyl)-3-(2-(4-hydroxypiperidin-1-yl)pyrimidin-5-yl)-8,9-dihydro-6H-pyridazino[1,2-a]indazol-11(7H)-one;
6-(2-(difluoromethoxy)phenyl)-3-(2-(1,1-dioxidothiomorpholino)pyrimidin-5-yl)-8,9-dihydro-6H-pyridazino[1,2-a]indazol-11(7H)-one;
3-(2-methoxyphenyl)-6-(2-morpholinopyrimidin-5-yl)-2,3-dihydropyrazolo[1,2-a]indazol-9(1H)-one;
(R)-6-(2-(2-hydroxypropan-2-yl)pyrimidin-5-yl)-3-(2-methoxyphenyl)-2,3-dihydropyrazolo[1,2-a]indazol-9(1H)-one;
(S)-6-(2-(2-hydroxypropan-2-yl)pyrimidin-5-yl)-3-(2-methoxyphenyl)-2,3-dihydropyrazolo[1,2-a]indazol-9(1H)-one;
2-methyl-6-(6-(2-morpholinopyrimidin-5-yl)-9-oxo-1,2,3,9-tetrahydropyrazolo[1,2-a]indazol-3-yl)benzonitrile;
6-(2-morpholinopyrimidin-5-yl)-3-phenyl-2,3-dihydropyrazolo[1,2-a]indazol-9(1H)-one;
4-methoxy-3-(6-(2-morpholinopyrimidin-5-yl)-9-oxo-1,2,3,9-tetrahydropyrazolo[1,2-a]indazol-3-yl)benzonitrile;
2-methoxy-3-(6-(2-morpholinopyrimidin-5-yl)-9-oxo-1,2,3,9-tetrahydropyrazolo[1,2-a]indazol-3-yl)benzonitrile;
3-(1-isopropyl-1H-pyrazol-5-yl)-6-(2-morpholinopyrimidin-5-yl)-2,3-dihydropyrazolo[1,2-a]indazol-9(1H)-one;
2-methyl-6-(6-(2-morpholinopyrimidin-5-yl)-9-oxo-1,2,3,9-tetrahydropyrazolo[1,2-a]indazol-3-yl)benzamide;
rac-(1R,9bR)-1-(2-(difluoromethoxy)phenyl)-8-(2-morpholinopyrimidin-5-yl)-2,3-dihydro-1H-pyrrolo[2,1-a]isoindol-5(9bH)-one;
rac-(1R,9bS)-1-(2-(difluoromethoxy)phenyl)-8-(2-morpholinopyrimidin-5-yl)-2,3-dihydro-1H-pyrrolo[2,1-a]isoindol-5(9bH)-one;
rac-(1R,10bR)-1-(2-(difluoromethoxy)phenyl)-9-(2-morpholinopyrimidin-5-yl)-3,4-dihydro-1H-[1,4]oxazino[3,4-a]isoindol-6(10bH)-one;
(1S,9bS)-1-(2-(difluoromethoxy)phenyl)-8-(2-morpholinopyrimidin-5-yl)-2,3-dihydro-1H-pyrrolo[2,1-a]isoindol-5(9bH)-one;
(1R,9bR)-1-(2-(difluoromethoxy)phenyl)-8-(2-morpholinopyrimidin-5-yl)-2,3-dihydro-1H-pyrrolo[2,1-a]isoindol-5(9bH)-one;
(1S,9bS)-1-(2-(difluoromethoxy)phenyl)-8-(2-(2-hydroxypropan-2-yl)pyrimidin-5-yl)-2,3-dihydro-1H-pyrrolo[2,1-a]isoindol-5(9bH)-one;
(1R,9bR)-1-(2-(difluoromethoxy)phenyl)-8-(2-(2-hydroxypropan-2-yl)pyrimidin-5-yl)-2,3-dihydro-1H-pyrrolo[2,1-a]isoindol-5(9bH)-one;
(1S,9bS)-1-(2-(difluoromethoxy)phenyl)-8-(2-((R)-2-(methoxymethyl)pyrrolidin-1-yl)pyrimidin-5-yl)-2,3-dihydro-1H-pyrrolo[2,1-a]isoindol-5(9bH)-one;
(1R,9bR)-1-(2-(difluoromethoxy)phenyl)-8-(2-((R)-2-(methoxymethyl)pyrrolidin-1-yl)pyrimidin-5-yl)-2,3-dihydro-1H-pyrrolo[2,1-a]isoindol-5(9bH)-one;
(1R,9bR)-1-(2-(difluoromethoxy)phenyl)-8-(2-((R)-3-oxohexahydroimidazo[1,5-a]pyrazin-7(1H)-yl)pyrimidin-5-yl)-2,3-dihydro-1H-pyrrolo[2,1-a]isoindol-5(9bH)-one;
(1R,9bR)-1-(2-(difluoromethoxy)phenyl)-8-(2-((S)-3-oxohexahydroimidazo[1,5-a]pyrazin-7(1H)-yl)pyrimidin-5-yl)-2,3-dihydro-1H-pyrrolo[2,1-a]isoindol-5(9bH)-one;
(1R)-1-(2-(difluoromethoxy)phenyl)-8-(2-(2-hydroxypropan-2-yl)-4-methylpyrimidin-5-yl)-2,3-dihydro-1H-pyrrolo[2,1-a]isoindol-5(9bH)-one;
(R)-6-(2-((R)-4-acetyl-2-methylpiperazin-1-yl)pyrimidin-5-yl)-3-(2-(difluoromethoxy)phenyl)-2,3-dihydropyrazolo[1,2-a]indazol-9(1H)-one;
(S)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one;
(R)-3-(2-(difluoromethoxy)-5-methylphenyl)-6-(2-(2-hydroxypropan-2-yl)pyrimidin-5-yl)-2,3-dihydropyrazolo[1,2-a]indazol-9(1H)-one;
(R)-6-(2-((R)-4-acetyl-3-methylpiperazin-1-yl)pyrimidin-5-yl)-3-(2-(difluoromethoxy)phenyl)-2,3-dihydropyrazolo[1,2-a]indazol-9(1H)-one;
(R)-3-(2-(difluoromethoxy)-5-methylphenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one;
(R)-3-(2-(difluoromethoxy)phenyl)-6-(2-((R)-4-(2-hydroxyacetyl)-3-methylpiperazin-1-yl)pyrimidin-5-yl)-2,3-dihydropyrazolo[1,2-a]indazol-9(1H)-one;
(R)-3-(2-(difluoromethoxy)-5-methylphenyl)-6-(2-(2-hydroxypropan-2-yl)pyrimidin-5-yl)-2,3-dihydropyrazolo[1,2-a]indazol-9(1H)-one;
(S)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one;
(S)-3-(2-(difluoromethoxy)phenyl)-6-(2-(2-hydroxypropan-2-yl)pyrimidin-5-yl)-2,3-dihydropyrazolo[1,2-a]indazol-9(1H)-one;
(R)-3-(2-(difluoromethoxy)phenyl)-6-(2-((R)-3-hydroxy-4-(2-hydroxyacetyl)piperazin-1-yl)pyrimidin-5-yl)-2,3-dihydro-1H,9H-pyrazolo[1,2-a]indazol-9-one;
(S)-3-(2-(difluoromethoxy)-5-methylphenyl)-7-fluoro-6-(2-(2-hydroxypropan-2-yl)pyrimidin-5-yl)-2,3-dihydropyrazolo[1,2-a]indazol-9(1H)-one;
(R)-3-(2-(difluoromethoxy)-5-methylphenyl)-7-fluoro-6-(2-(2-hydroxypropan-2-yl)pyrimidin-5-yl)-2,3-dihydropyrazolo[1,2-a]indazol-9(1H)-one;
(R)-3-(2-(difluoromethoxy)phenyl)-7-fluoro-6-(2-(2-hydroxypropan-2-yl)pyrimidin-5-yl)-2,3-dihydropyrazolo[1,2-a]indazol-9(1H)-one;
(S)-3-(2-(difluoromethoxy)phenyl)-7-fluoro-6-(2-(2-hydroxypropan-2-yl)pyrimidin-5-yl)-2,3-dihydropyrazolo[1,2-a]indazol-9(1H)-one;
3-(5-(hydroxymethyl)-2-methoxyphenyl)-6-(2-morpholinopyrimidin-5-yl)-2,3-dihydropyrazolo[1,2-a]indazol-9(1H)-one;
(R)-3-(2-(difluoromethoxy)-5-methylphenyl)-7-fluoro-6-(2-(2-hydroxypropan-2-yl)pyrimidin-5-yl)-2,3-dihydro-1H,9H-pyrazolo[1,2-a]indazol-9-one;
(S)-3-(2-(difluoromethoxy)-5-methylphenyl)-7-fluoro-6-(2-(2-hydroxypropan-2-yl)pyrimidin-5-yl)-2,3-dihydro-1H,9H-pyrazolo[1,2-a]indazol-9-one;
(S)-3-(2-(difluoromethoxy)phenyl)-6-(2-(2-hydroxypropan-2-yl)pyrimidin-5-yl)-2,3-dihydro-1H,9H-pyrazolo[1,2-a]indazol-9-one;
(R)-3-(2-(difluoromethoxy)-5-methylphenyl)-6-(2-((S)-3-oxohexahydroimidazo[1,5-a]pyrazin-7(1H)-yl)pyrimidin-5-yl)-2,3-dihydro-1H,9H-pyrazolo[1,2-a]indazol-9-one;
(R)-3-(2-(difluoromethoxy)-5-methylphenyl)-6-(2-(2-hydroxypropan-2-yl)pyrimidin-5-yl)-2,3-dihydro-1H,9H-pyrazolo[1,2-a]indazol-9-one;
(R)-6-(2-((R)-4-acetyl-3-methylpiperazin-1-yl)pyrimidin-5-yl)-3-(2-(difluoromethoxy)phenyl)-2,3-dihydropyrazolo[1,2-a]indazol-9(1H)-one;
(R)-3-(2-(difluoromethoxy)phenyl)-6-(2-((R)-3-hydroxy-4-(2-hydroxyacetyl)piperazin-1-yl)pyrimidin-5-yl)-2,3-dihydropyrazolo[1,2-a]indazol-9(1H)-one;
9b-(2-methoxyphenyl)-8-(2-morpholinopyrimidin-5-yl)-2,3-dihydro-1H-pyrrolo[2,1-a]isoindol-5(9bH)-one; or
3-(5-(hydroxymethyl)-2-methoxyphenyl)-6-(2-morpholinopyrimidin-5-yl)-2,3-dihydropyrazolo[1,2-a]indazol-9(1H)-one.

### AATM

Any autoantibody targeting moiety (AATM) that binds to an autoantibody is useful within the present disclosure. In certain non-limiting embodiments, the autoantibody is pathological. Any autoantibodies known in the art is contemplated within the present disclosure.

In certain embodiments, the AATM is any peptide and/or small molecule that binds to FcRn, as known in the art or described elsewhere herein.

In certain embodiments, the AATM comprises a FcRn antagonist, such as but not limited to rozanolixizumab (see, for example, Kiessling, et al., 2017, Sci. Transl. Med. 9:eaan1208).

In certain embodiments, the AATM comprises a FcRn antagonist, such as but not limited to efgartigimod (see, for example, Ulrichts, et al., 2018, J. Clin. Invest. 128(10):4372).

In certain embodiments, the AATM comprises 2,4-dinitrobenzene or any derivative or analogue thereof (wherein the phenyl ring is optionally substituted):

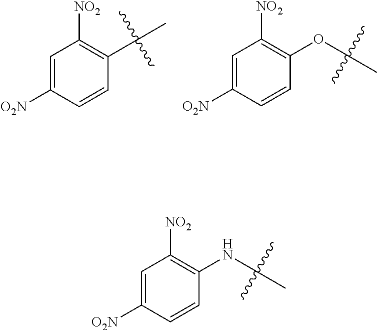 [SMILES: *c1ccc([N+](=O)[O-])cc1[N+](=O)[O-] ; *Oc1ccc([N+](=O)[O-])cc1[N+](=O)[O-] ; *Nc1ccc([N+](=O)[O-])cc1[N+](=O)[O-]]

In certain embodiments, the AATM comprises the following cyclic peptide FcIII, or any reduced form thereof (e.g., any corresponding free thiol derivative thereof; see for example Science 2000, 287:1279-1283). The chemical group marked with * is a non-limiting position for attachment of Linker or CON in the compound of the disclosure.

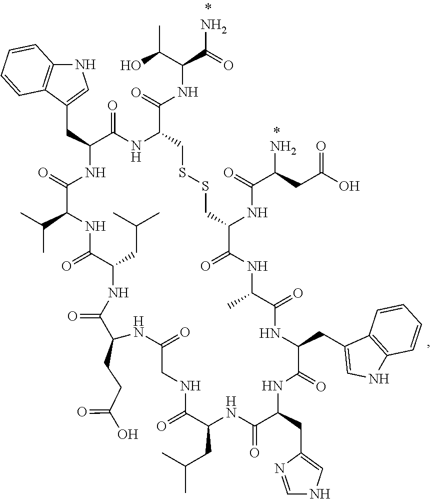 [SMILES: CC(C)C[C@@H]1NC(=O)[C@H](Cc2c[nH]cn2)NC(=O)[C@H](Cc2c[nH]c3ccccc23)NC(=O)[C@H](C)NC(=O)[C@@H](NC(=O)[C@@H](N)CC(=O)O)CSSC[C@@H](C(=O)N[C@H](C(N)=O)[C@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC1=O]

also represented as

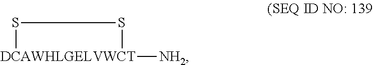

internal cystine form with C-terminus amidated).

In certain embodiments, the AATM comprises the following cyclic peptide FcIII-4C (amide), or any reduced form thereof (e.g., any corresponding free thiol derivative thereof: see Bioconjugate Chem. 2016, 27:1569). The chemical group marked with * is a non-limiting position for attachment of Linker or CON in the compound of the disclosure.

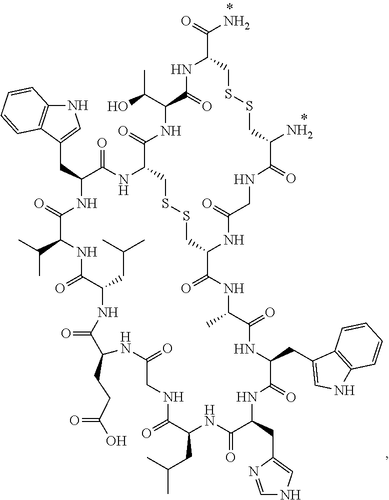 [SMILES: CC(C)C[C@@H]1NC(=O)[C@H](Cc2c[nH]cn2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@H](C)O)C(=O)N[C@H](C(N)=O)CSSC[C@H](N)C(=O)NCC(=O)N2]

also represented as

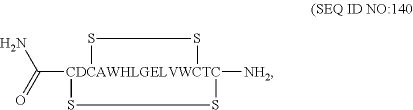

internal cystine form with C-terminus amidated).

In certain embodiments, the AATM comprises a compound of formula (3a) or (3b):

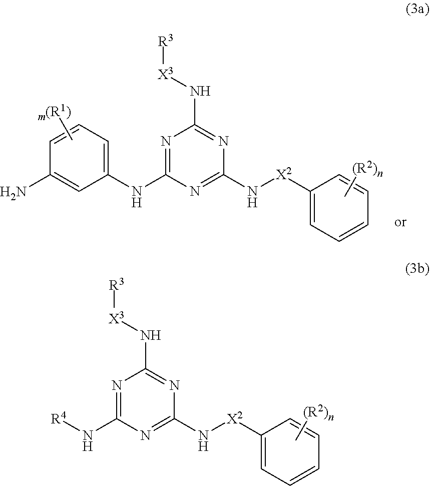

wherein:
each occurrence of R¹ is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N (R)₂, —C(═O)R, —C(═O) OR, —OC(═O)R, —C(═O) N (R)₂, —N(R) S(═O)₂R, —N(R)C(═O)OR, —N(R)C(═O)R, and —N(R)C(═O) N (R)₂, wherein each occurrence of R is independently H, C₁-C₆ alkyl, or C₃-C₈ cycloalkyl;
m is 0, 1, 2, 3, or 4;
X² is a bond, optionally substituted C₁-C₆ alkyl, or optionally substituted C₁-C₆ heteroalkyl; each occurrence of R² is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
n is 0, 1, 2, 3, or 4;
X³ is a bond, optionally substituted C₁-C₆ alkyl, or optionally substituted C₁-C₆ heteroalkyl;
R³ is H, R, —OH, —NH₂, —NHR, —C(═O)OH, or —SH, wherein each occurrence of R is C₁-C₆ alkyl or C₃-C₈ cycloalkyl;
R⁴ is cycloalkyl, including polycyclic cycloalkyl, which is optionally substituted with 1-4 groups independently selected from the group consisting of F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
wherein the AATM is linked to Linker or CON through R³ or at least one of R′ or R².

In certain embodiments, the AATM comprises one of the following compounds (see WO 2006/024175 A1). Each of the chemical groups marked with * illustrates a non-limiting position for attachment of Linker or CON in the compound of the disclosure.

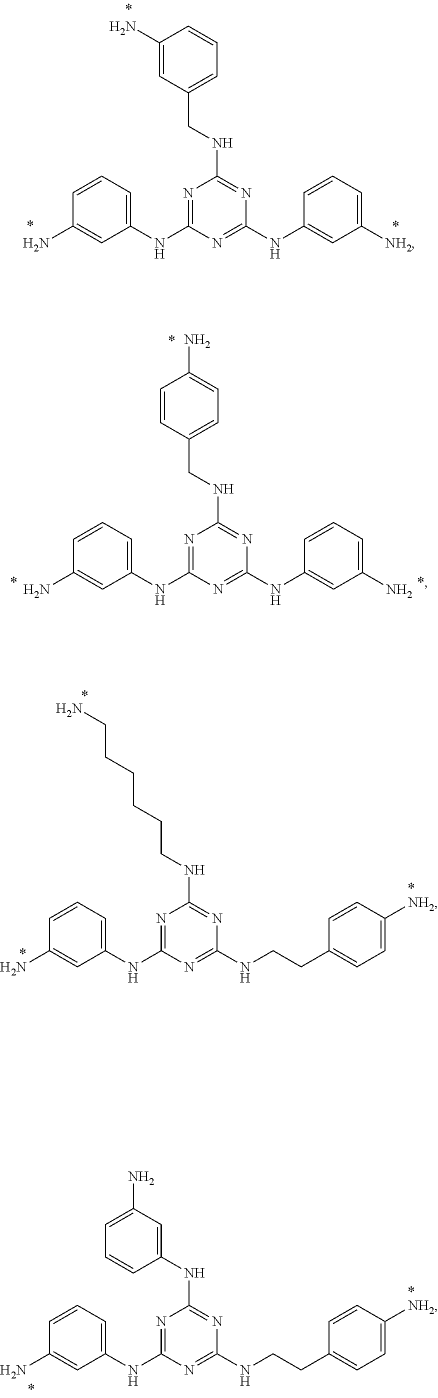 [SMILES: Nc1cccc(CNc2nc(Nc3cccc(N)c3)nc(Nc3cccc(N)c3)n2)c1 ; Nc1ccc(CNc2nc(Nc3cccc(N)c3)nc(Nc3cccc(N)c3)n2)cc1 ; NCCCCCCNc1nc(NCCc2ccc(N)cc2)nc(Nc2cccc(N)c2)n1 ; Nc1ccc(CCNc2nc(Nc3cccc(N)c3)nc(Nc3cccc(N)c3)n2)cc1]

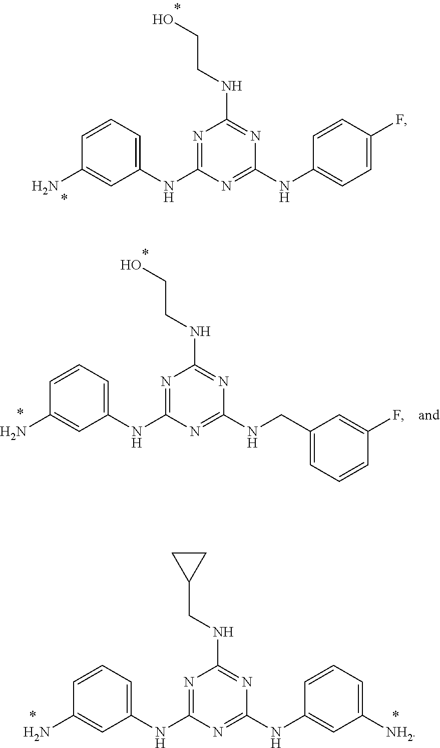 [SMILES: Nc1cccc(Nc2nc(NCCO)nc(Nc3ccc(F)cc3)n2)c1 ; Nc1cccc(Nc2nc(NCCO)nc(NCc3cccc(F)c3)n2)c1 ; "and" ; Nc1cccc(Nc2nc(NCC3CC3)nc(Nc3cccc(N)c3)n2)c1]

In certain embodiments, the AATM comprises the following compound, wherein the chemical bond marked with [symbol] non-limiting position for attachment of Linker or CON in the compound of the disclosure (see Chemistry & Biology 18:1179-1188).

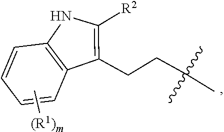

wherein:
each occurrence of R¹ is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
m is 0, 1, 2, 3, or 4;
each occurrence of R² is independently H, F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl.

The disclosures of U.S. Pat. No. 9,879,016 B2 and U.S. Patent Application No. US 2016/0304526 A1 are incorporated herein in their entireties by reference.

### Additional Galactose- and Talose-Based ASGPR Binding Moieties

In certain embodiments, the present disclosure is directed to compounds which are useful for removing circulating proteins which are associated with a disease state or condition in a patient or subject according to the general chemical structure of Formula II:

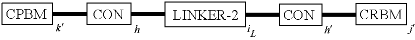

### Formula II

The term “Extracellular Protein Targeting Ligand” as used herein is interchangeably used with the term CPBM (cellular protein binding moiety). The term “ASGPR Ligand” as used herein is interchangeably used with an asialoglycoprotein receptor (ASGPR) binding moiety as defined herein.

In the compound of Formula II, each [CON] is an optional connector chemical moiety which, when present, connects directly to [CPBM] or to [CRBM] or connects the [LINKER-2] to [CPBM] or to [CRBM].

In the compound of Formula II:
[LINKER-2] is a chemical moiety having a valency from 1 to 15 which covalently attaches to one or more [CRBM] and/or [CPBM] group, optionally through a [CON], including a [MULTICON] group, wherein said [LINKER-2] optionally itself contains one or more [CON] or [MULTICON] group(s);
k′ is an integer from 1 to 15;
j′ is an integer from 1 to 15;
h and h′ are each independently an integer from 0 to 15;
i_{L} is an integer from 0 to 15;
with the proviso that at least one of h, h′ and i_{L} is at least 1, or a pharmaceutically acceptable salt, stereoisomer, solvate or polymorph thereof
A [MULTICON] group can connect one or more of a [CRBM] or [CPBM] to one or more of a [LINKER-2]. In various embodiments, [LINKER-2] has a valency of 1 to 10. In various embodiments, [LINKER-2] has a valency of 1 to 5. In various embodiments, [LINKER-2] has a valency of 1, 2 or 3. In various embodiments, in the compound of Formula II, the [LINKER-2] includes one or more of Linker^{A}, Linker^{B}, Linker^{C}, Linker^{D}, and/or combinations thereof as defined herein.

In the compound of Formula II, xx is independently selected from 0, 1, 2, 3, 4, 5, 6, 7, 8, 9,10,11, 12,13,14,15,16, 17,18, 19, 20, 21, 22, 23, 24, and 25.

In the compound of Formula II, yy is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, zz is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, X¹ is 1 to 5 contiguous atoms independently selected from O, S, N(R^{b}), and C(R⁴)(R⁴), wherein if X¹ is 1 atom then X¹ is O, S, N(R′), or C(R⁴)(R⁴), if X¹ is 2 atoms then no more than 1 atom of X¹ is O, S, or N(R⁴), if X¹ is 3, 4, or 5 atoms then no more than 2 atoms of X¹ are O, S, or N(R)
R³ at each occurrence is independently selected from hydrogen, alkyl, heteroalkyl, haloalkyl (including —CF₃, —CHF₂, —CH₂F, —CH₂CF₃, —CH₂CH₂F, and —CF₂CF₃), arylalkyl, heteroarylalkyl, alkenyl, alkynyl, and, heteroaryl, heterocycle, —OR⁸, and —NR⁸R⁹;
R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷,
R⁶ and R⁷ are independently selected at each occurrence from hydrogen, heteroalkyl, alkyl, arylalkyl, heteroaryl alkyl, alkenyl, alkynyl, and, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, heteroalkyl, alkyl, arylalkyl, heteroarylalkyl, alkenyl, alkynyl, aryl, heteroaryl, and heterocycle.

### A. Galactose-Based ASGPR-Binding Cellular Receptor Binding Moieties of Formula II

In certain embodiments, the compound of Formula II is selected from:

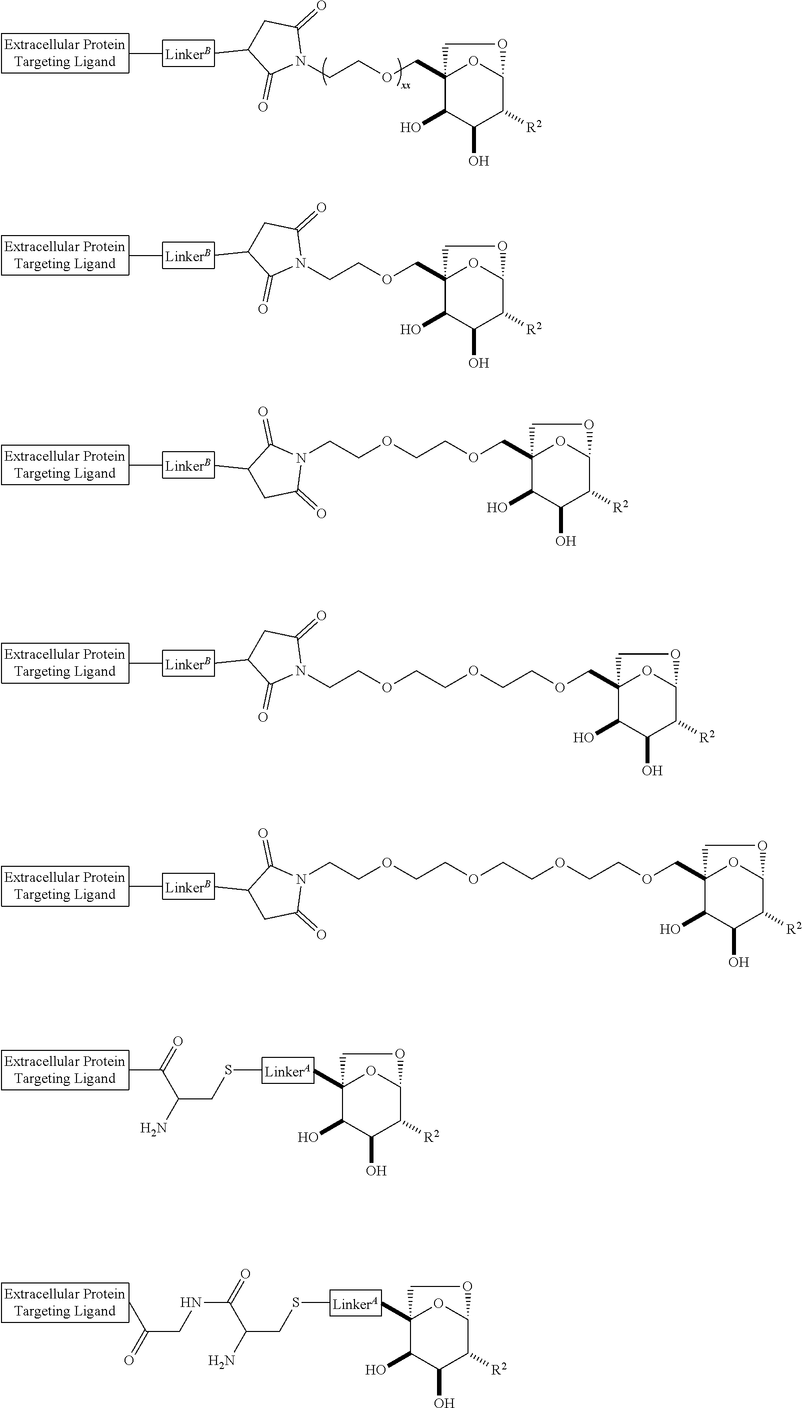

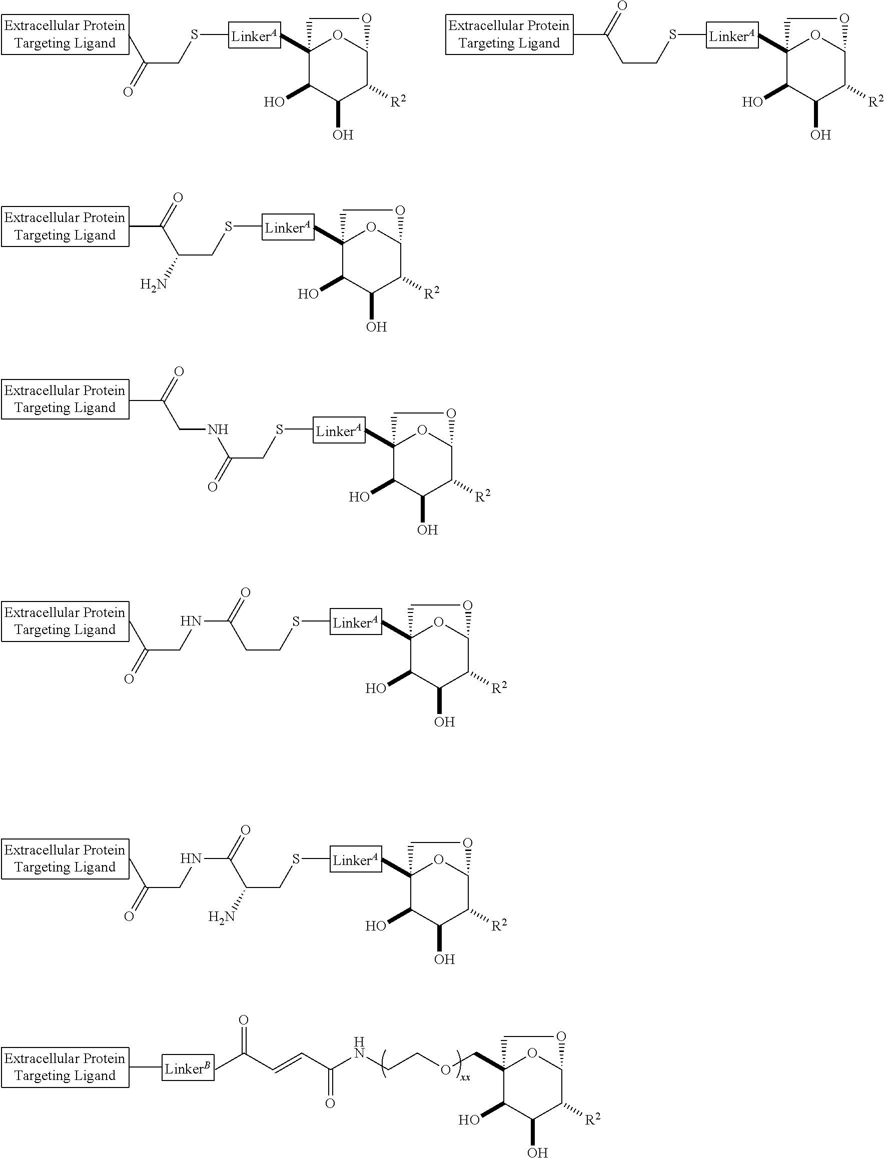

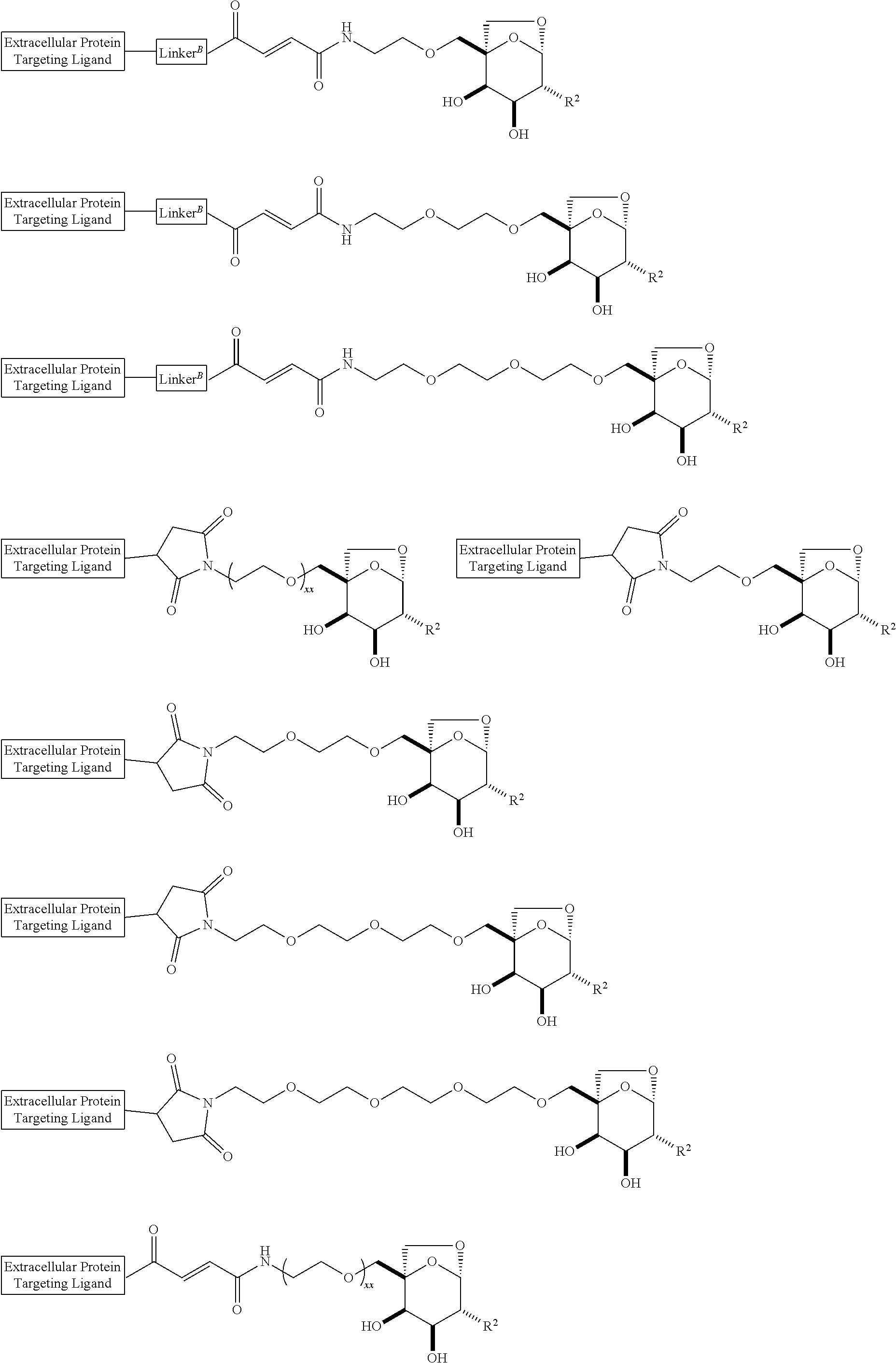

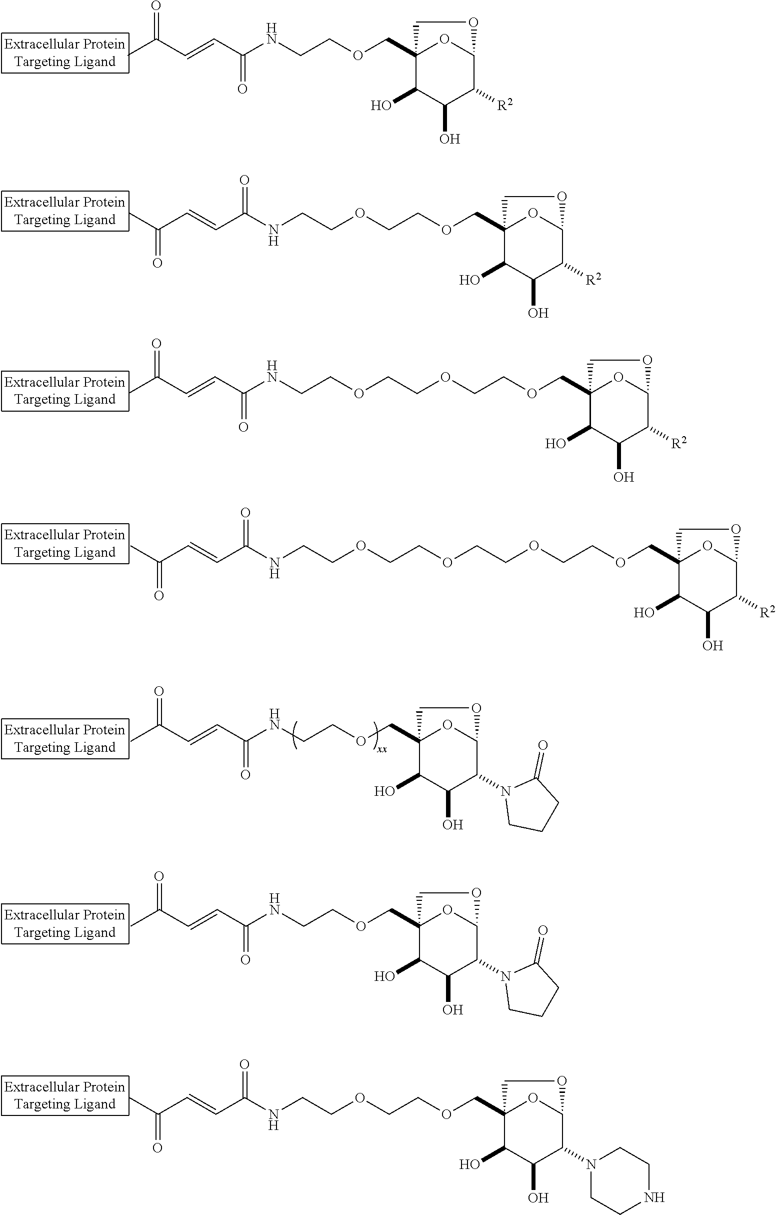

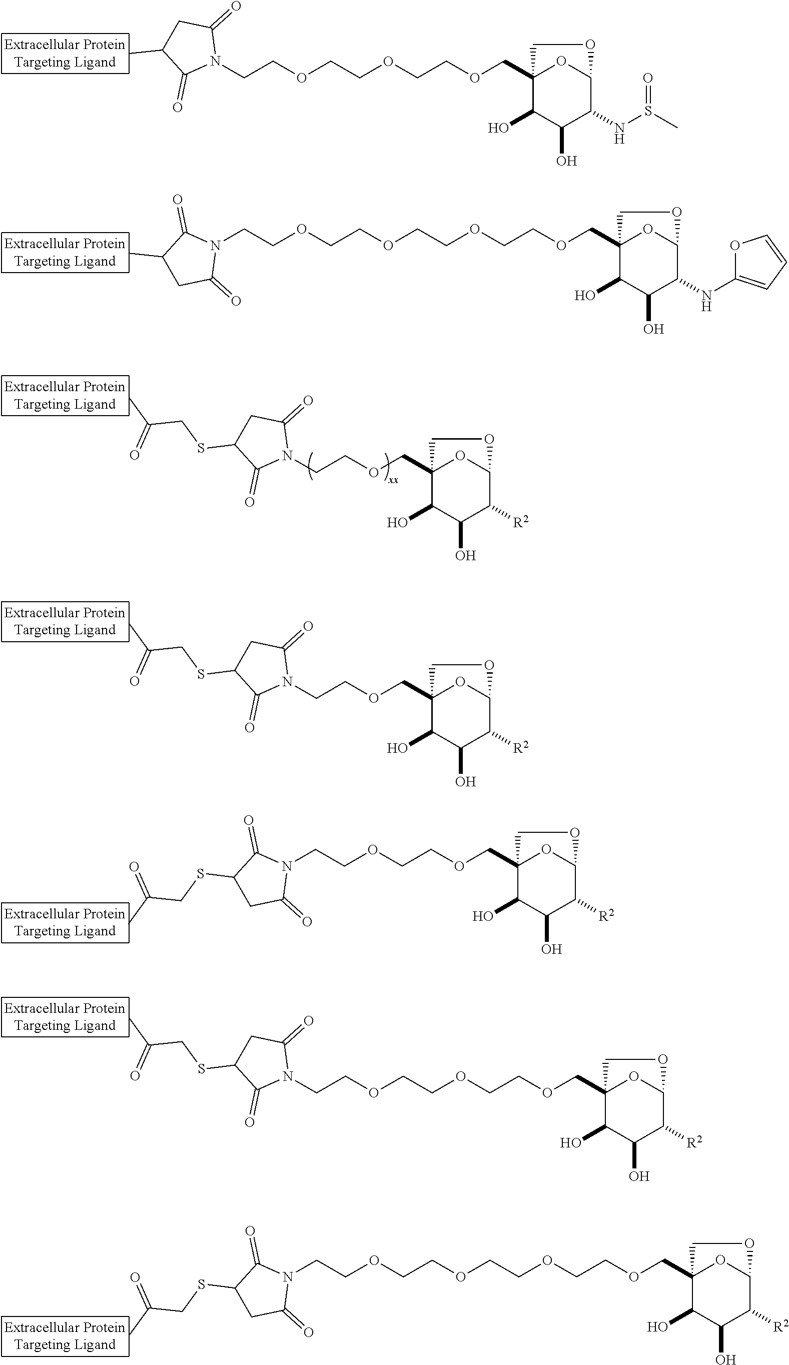

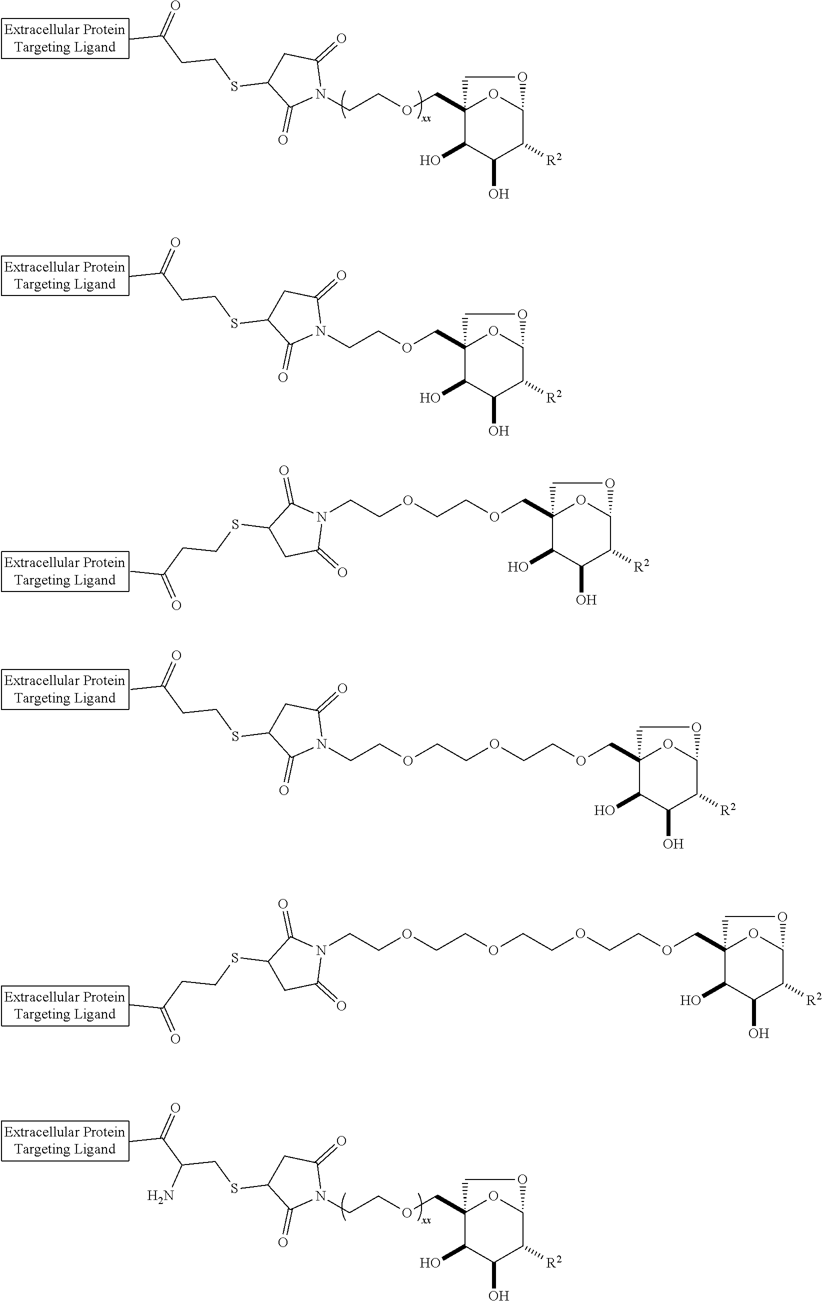

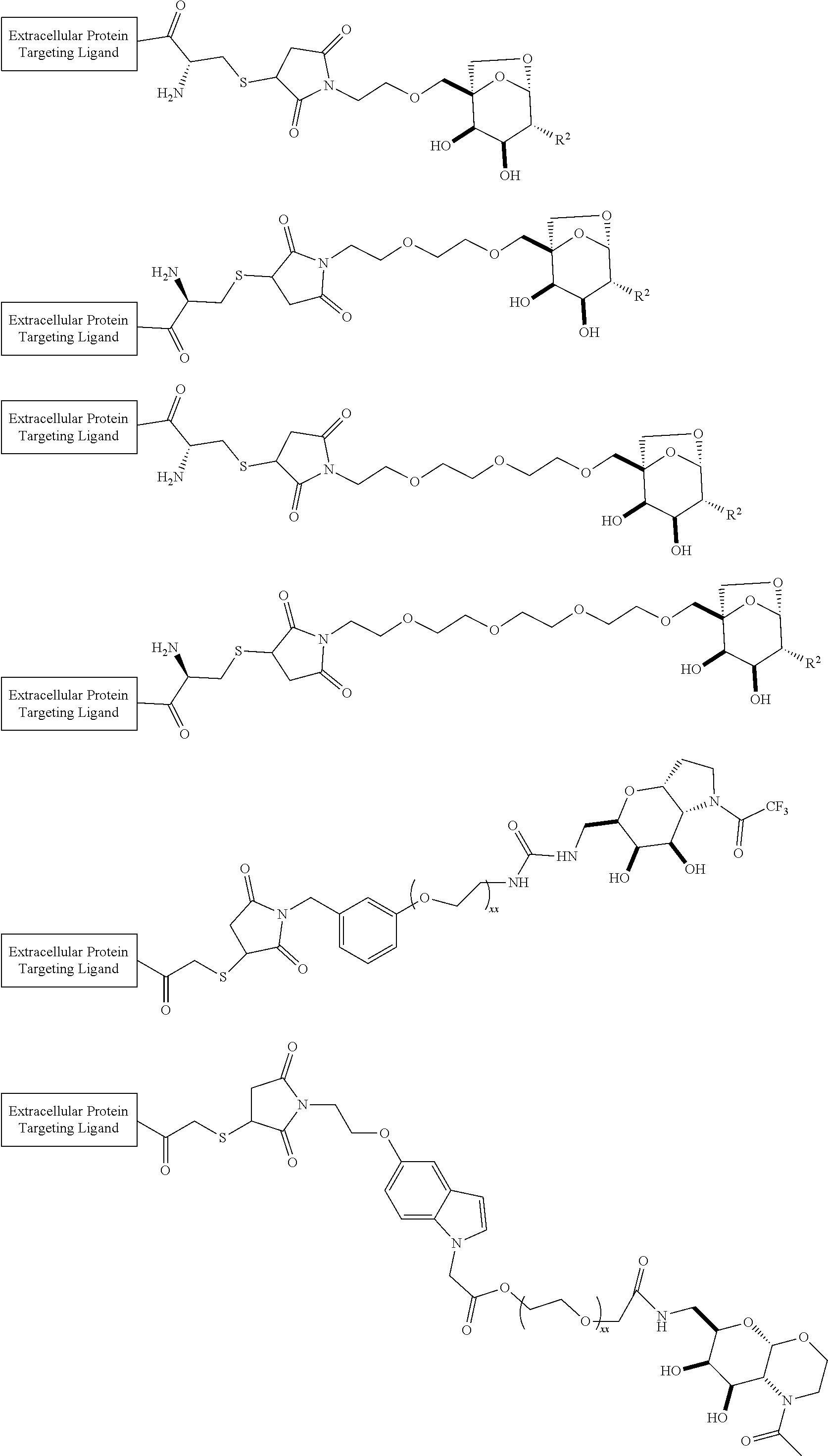

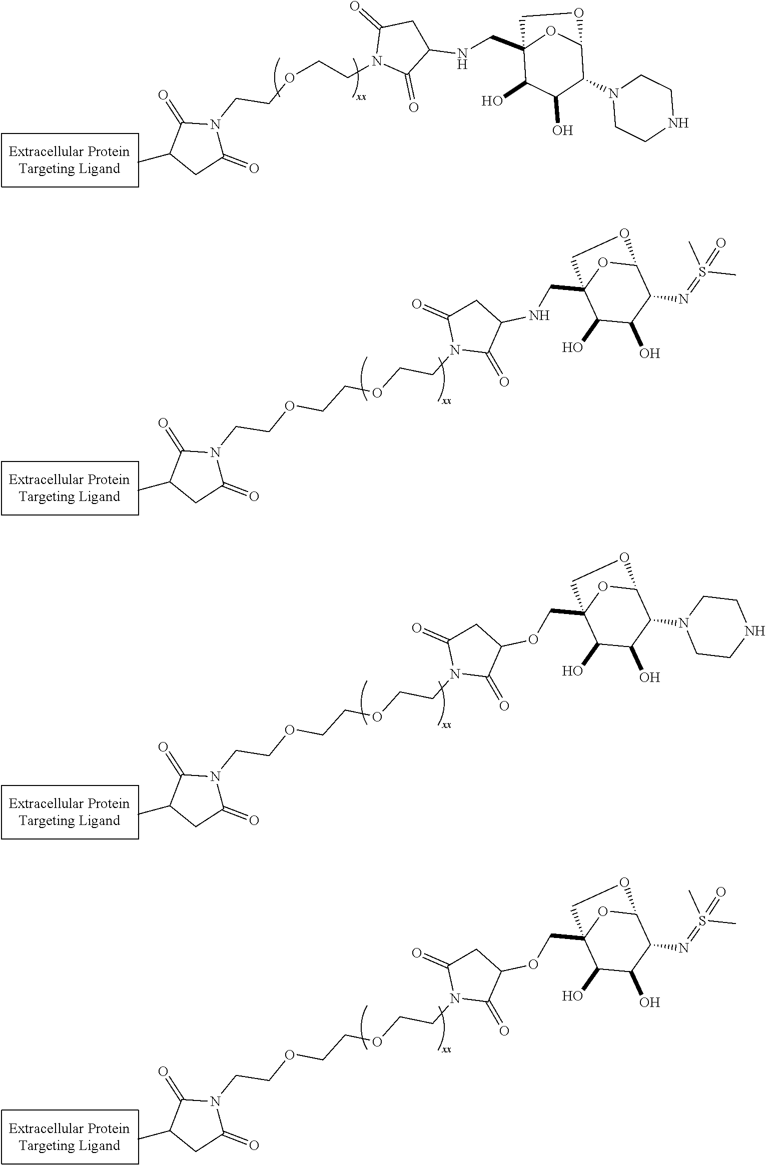

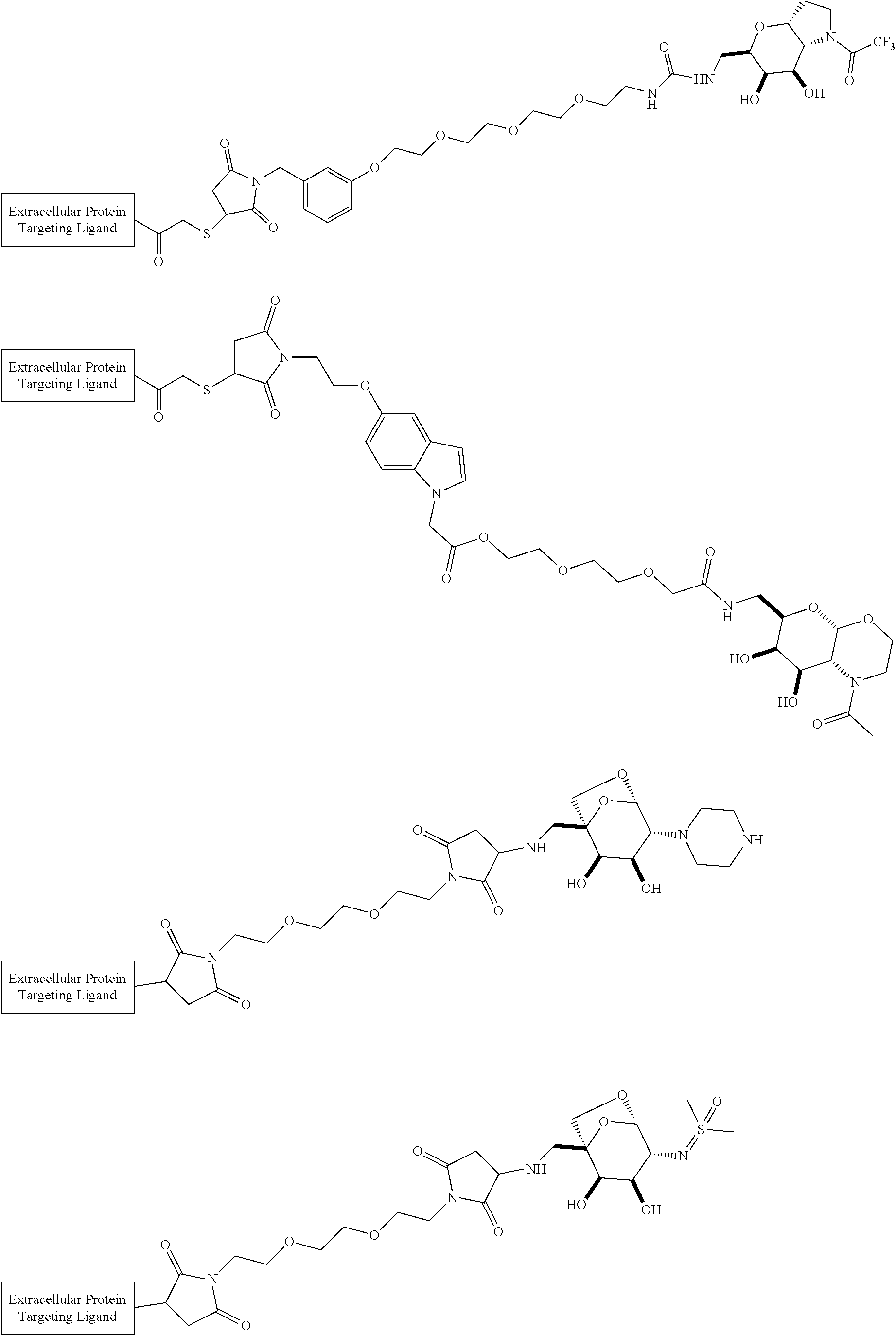

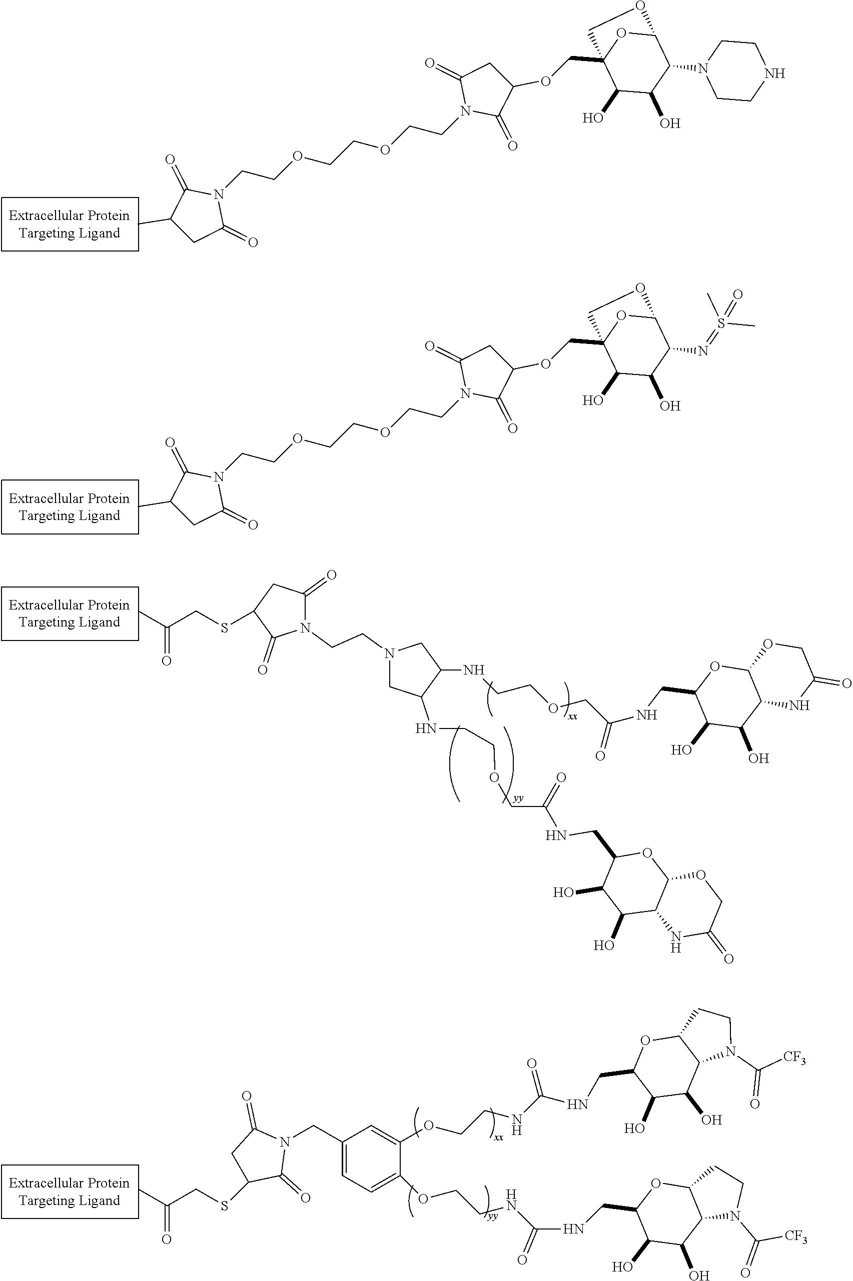

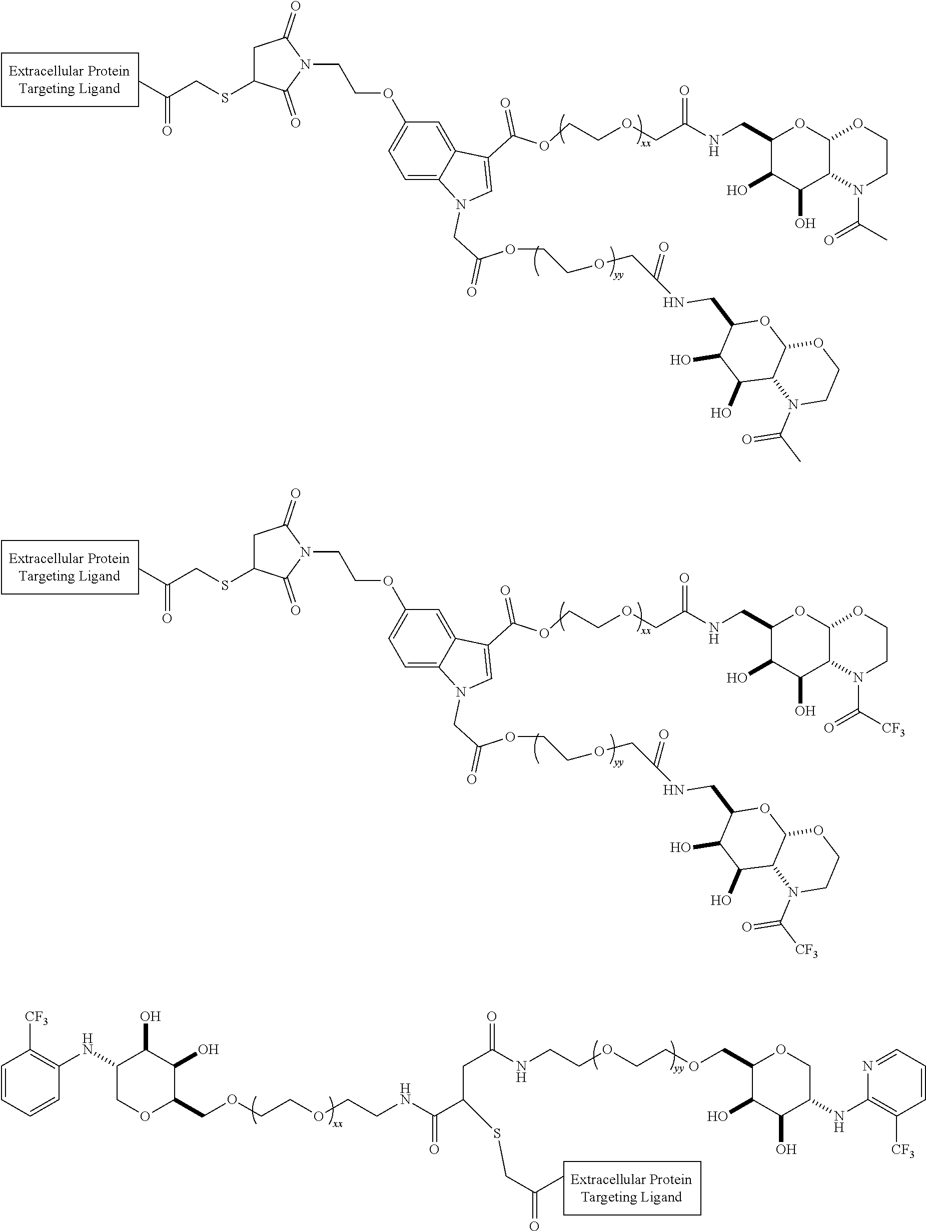

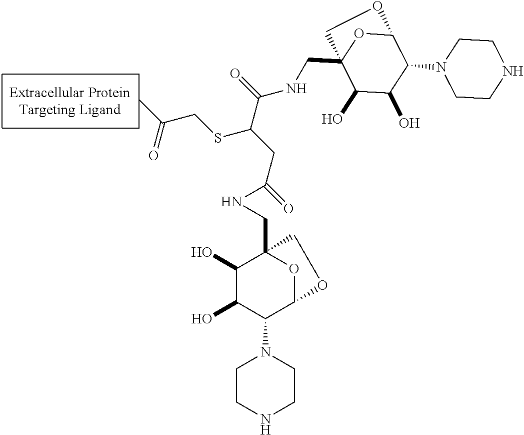

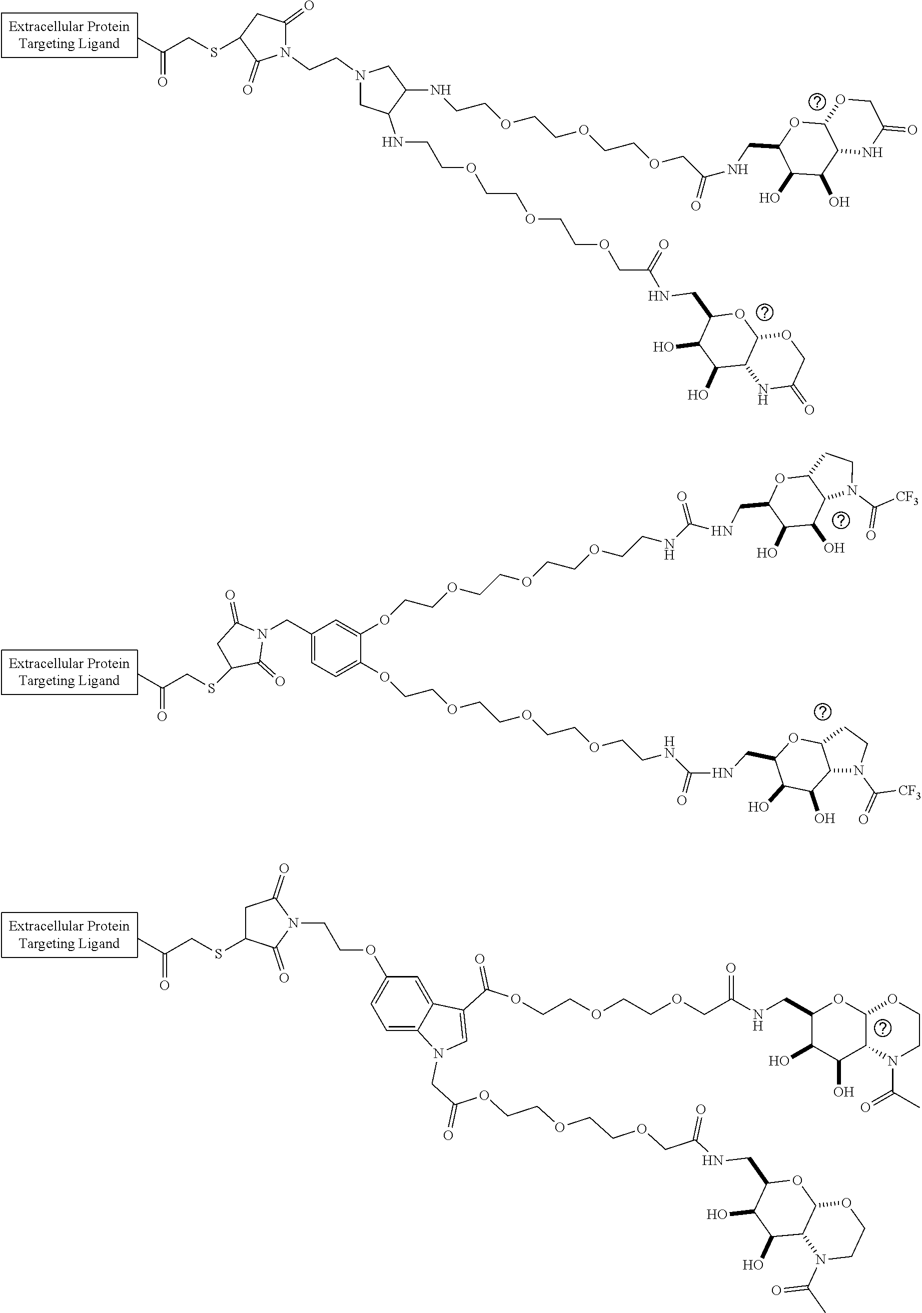

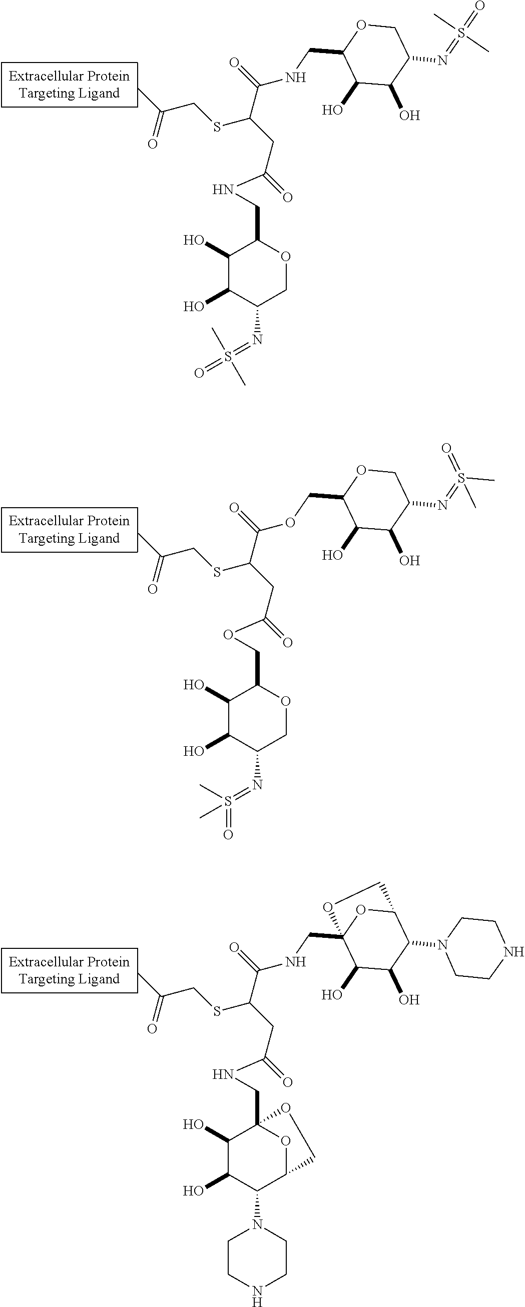

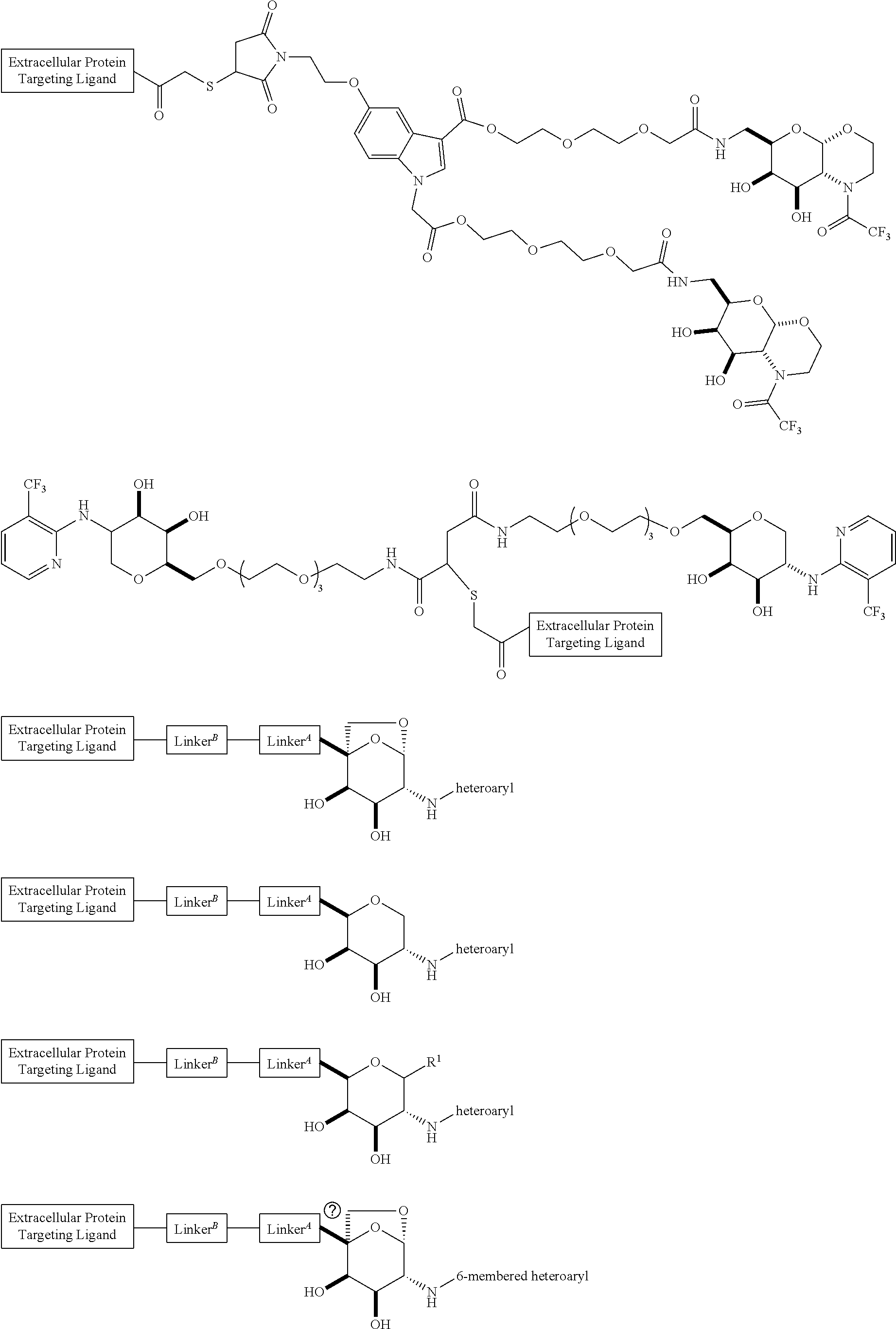

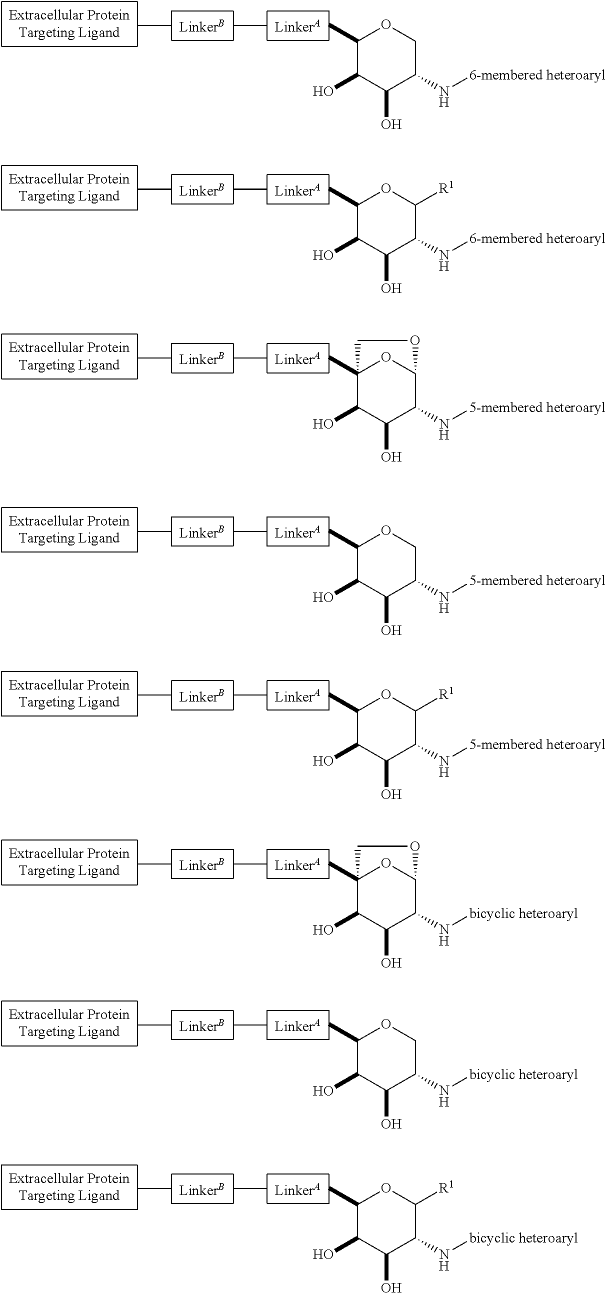

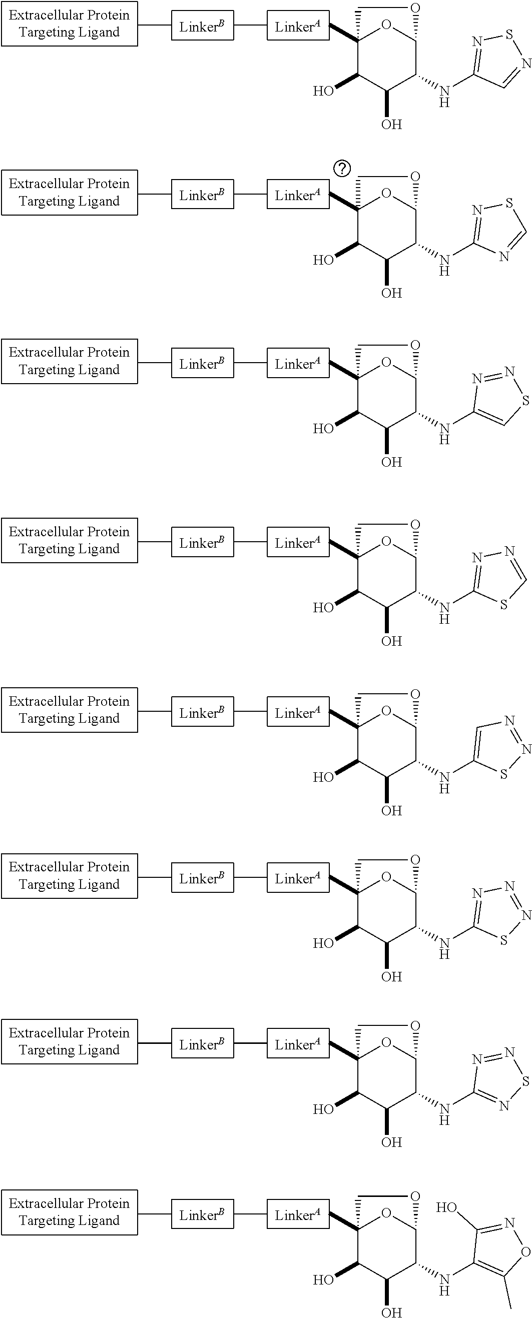

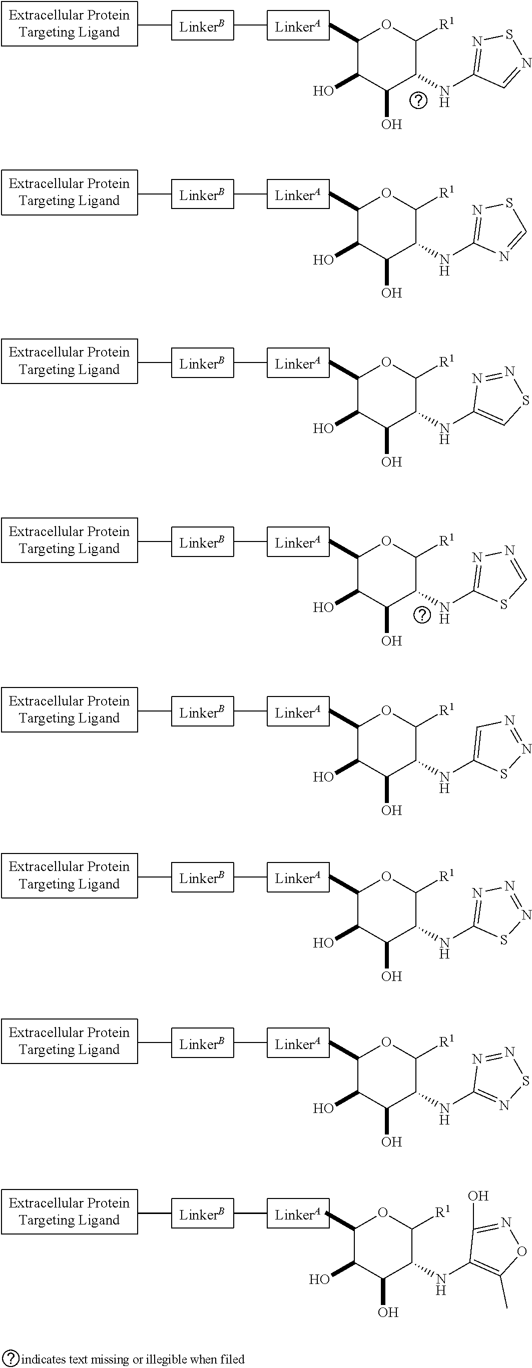

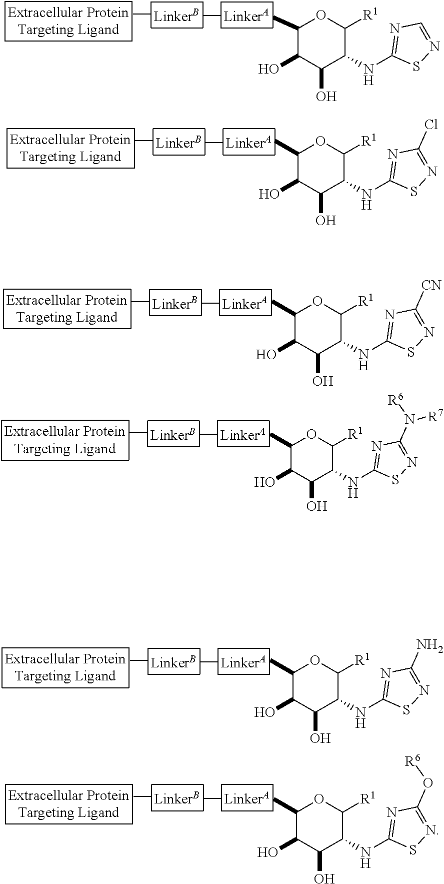

In certain embodiments, the compound of Formula II has one of the following structures:

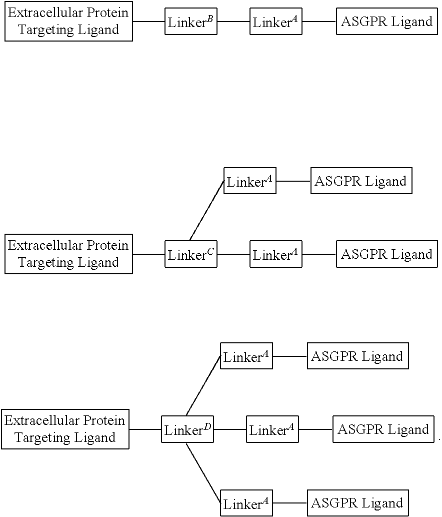

In various embodiments, the ASGPR ligand is linked at either the C¹ or C⁵ (R¹ or R⁵) position to form a degrading compound. In various embodiments, the ASGPR ligand is linked at C⁶ position to form a degrading compound. For example, when the ASGPR ligand is

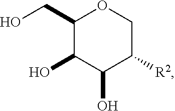

then non-limiting examples of ASGPR binding compounds of Formula II include:

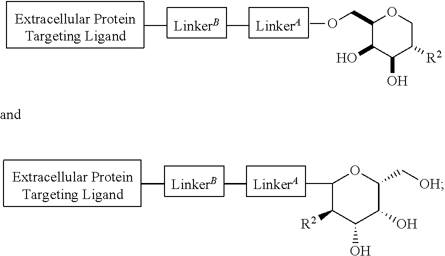

or the bi- or tri-substituted versions thereof or pharmaceutically acceptable salts thereof, where the bi- or tri-substitution refers to the number additional galactose derivatives attached to a linker moiety.

In any of the embodiments herein where an ASGPR ligand is drawn for use in a degrader the ASGPR ligand is typically linked through to the Extracellular Protein Targeting Ligand in the C⁵ position (e.g., which can refer to the adjacent C⁶ carbon hydroxyl or other functional moiety that can be used for linking purposes). When the linker and Extracellular Protein Targeting Ligand is connected through the C¹ position, then that carbon is appropriately functionalized for linking, for example with a hydroxyl, amino, allyl, alkyne or hydroxyl-allyl group.

In various embodiments, the ASGPR ligand is not linked in the C³ or C⁴ position, because these positions chelate with the calcium for ASGPR binding in the liver. In certain embodiments, an ASGPR ligand useful for incorporation into a compound of Formula II is selected from:

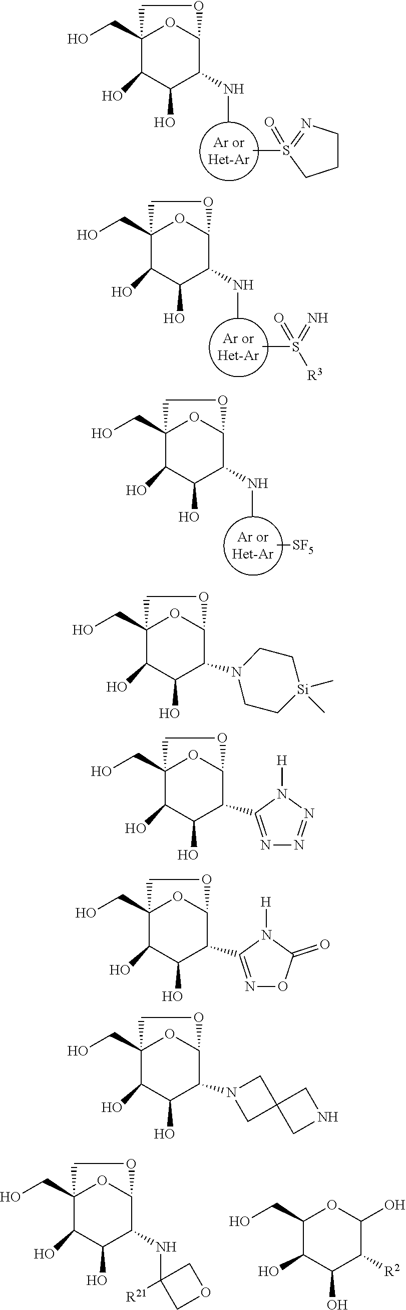

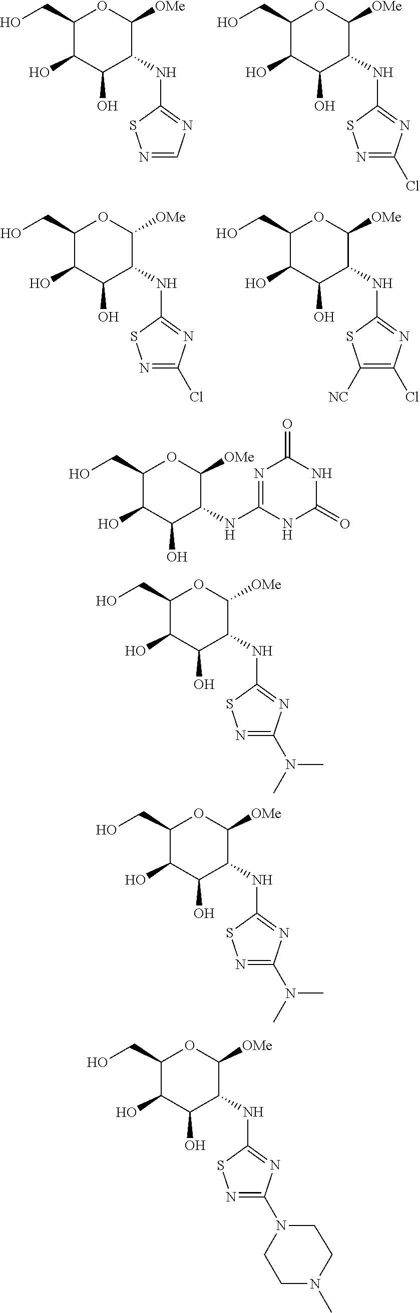

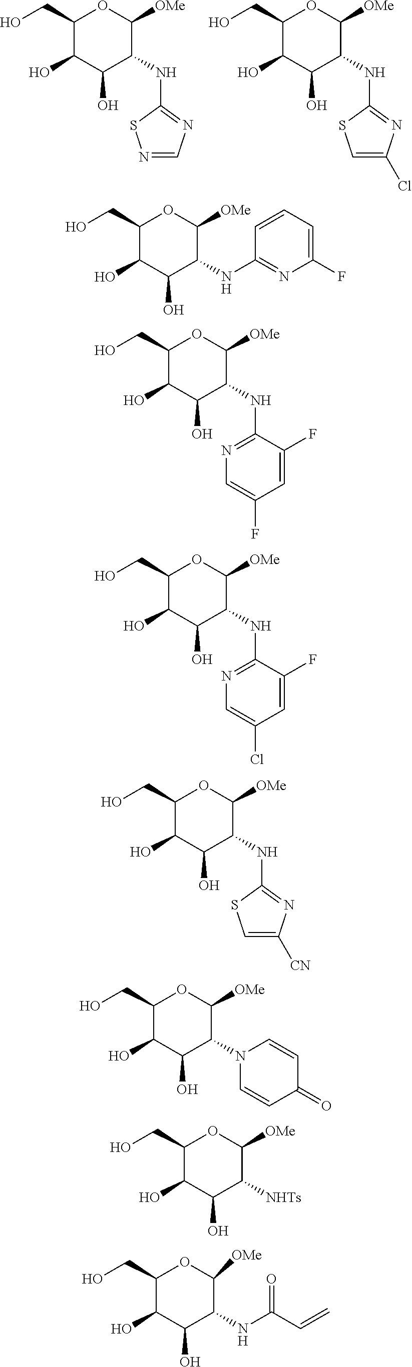

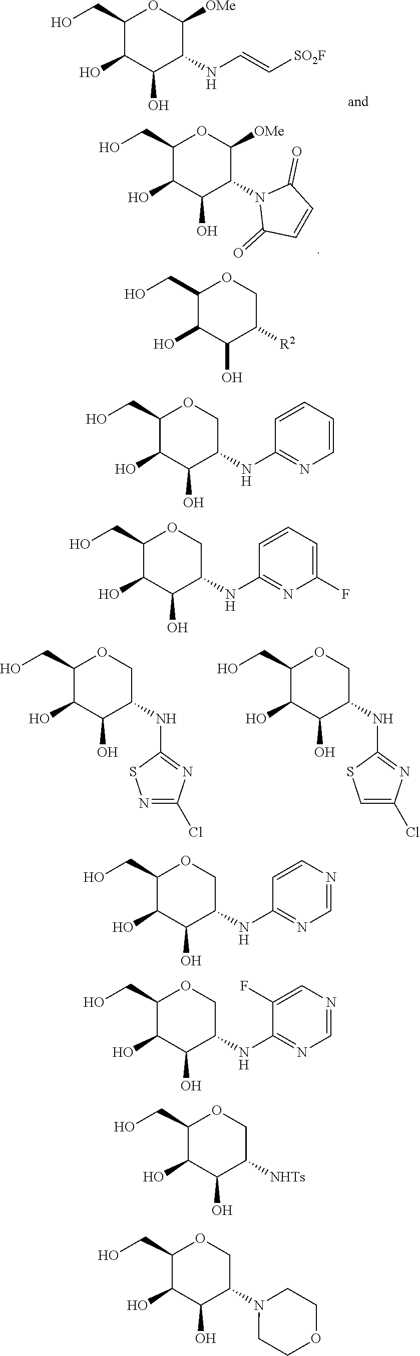

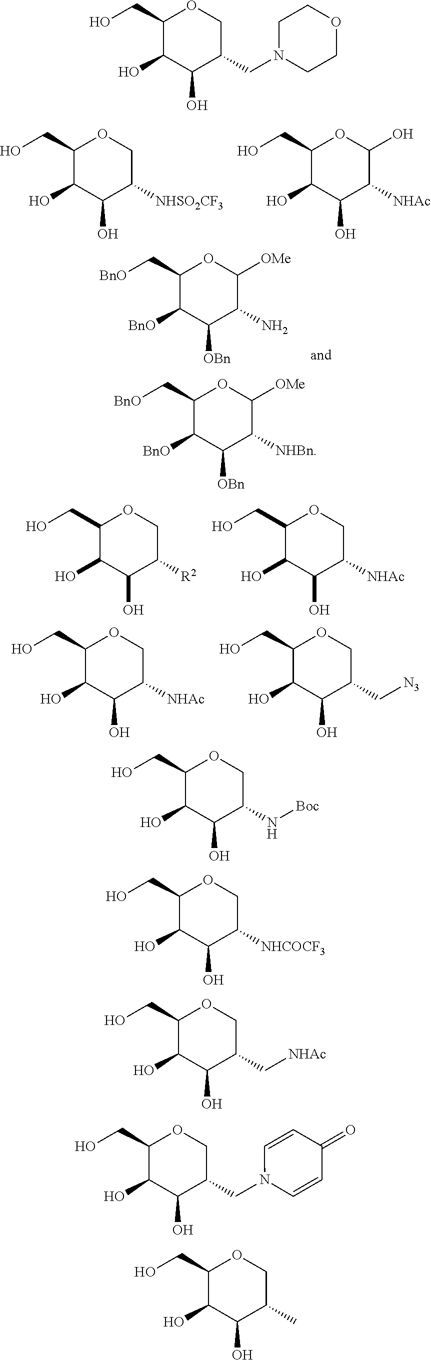

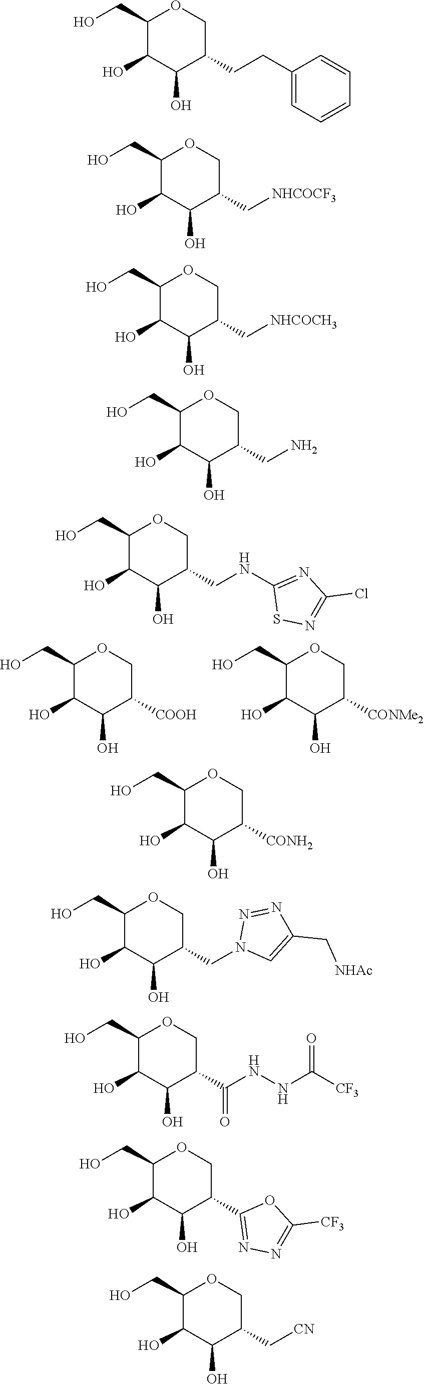

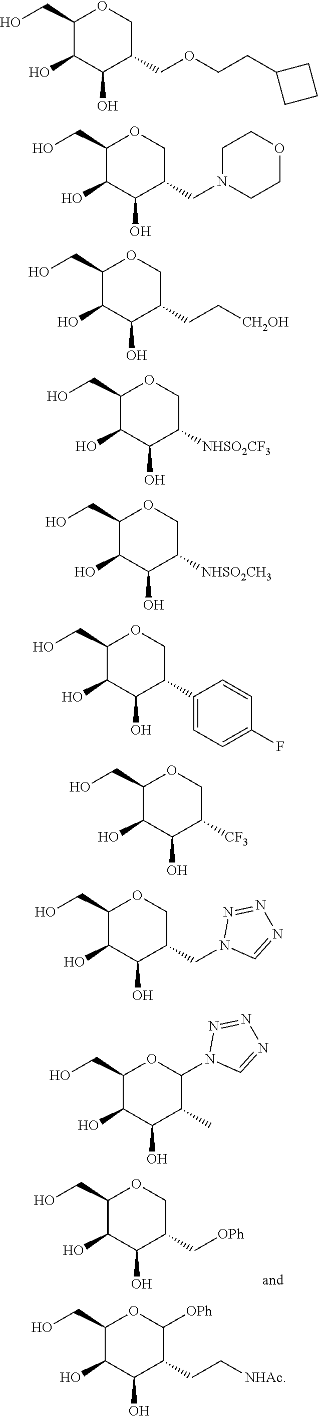

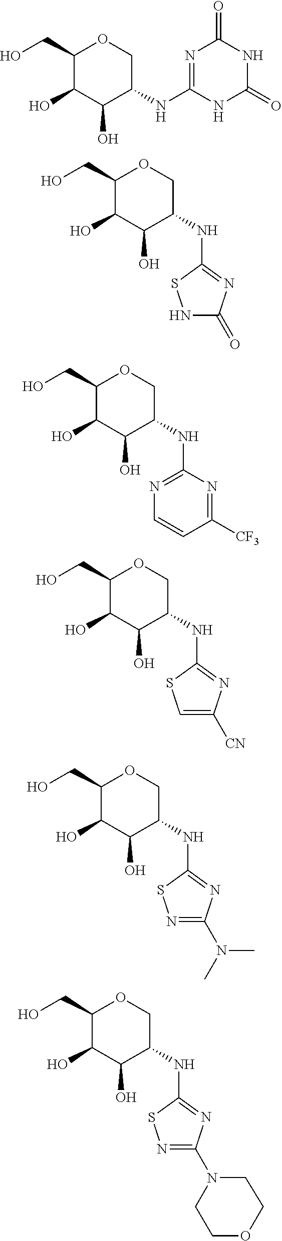

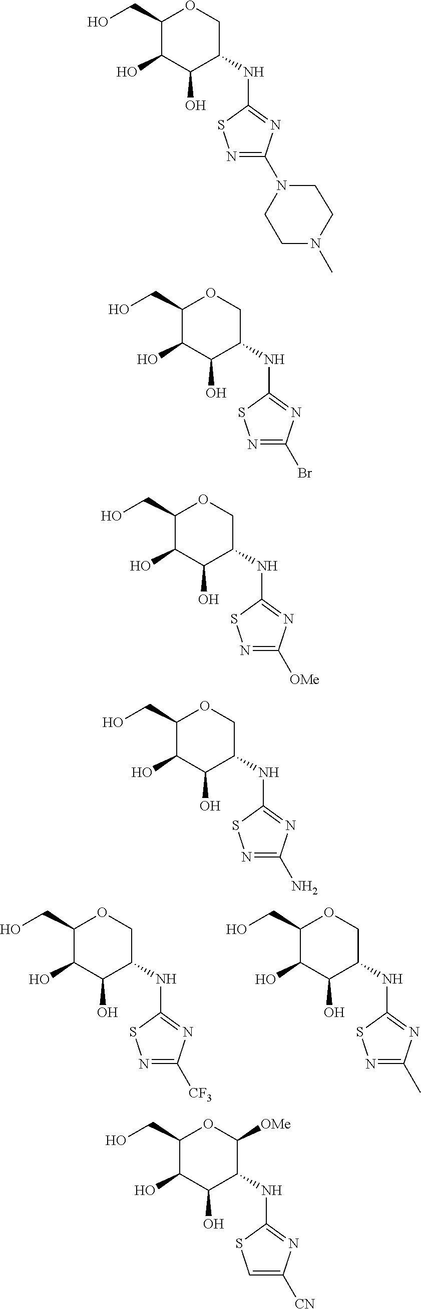

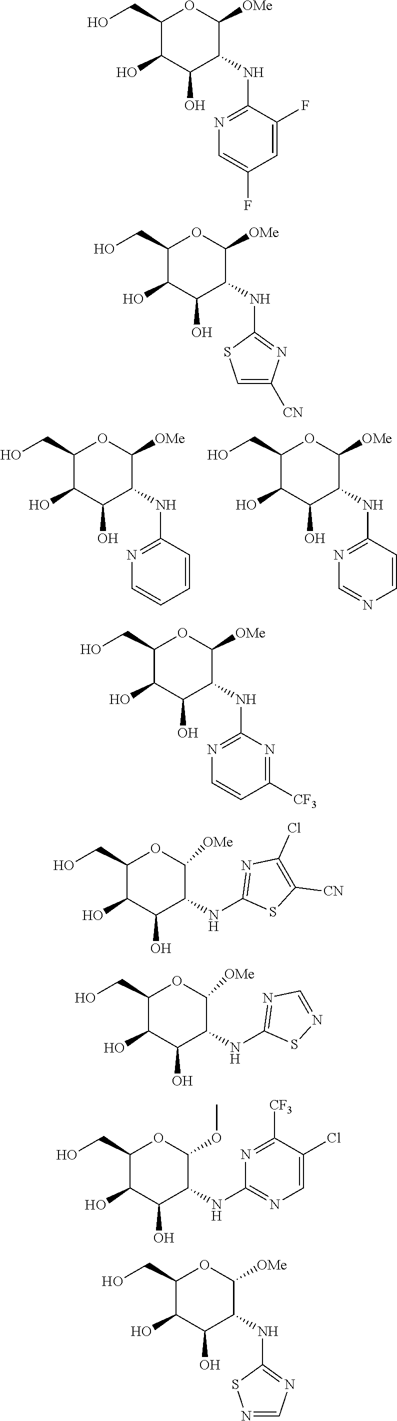

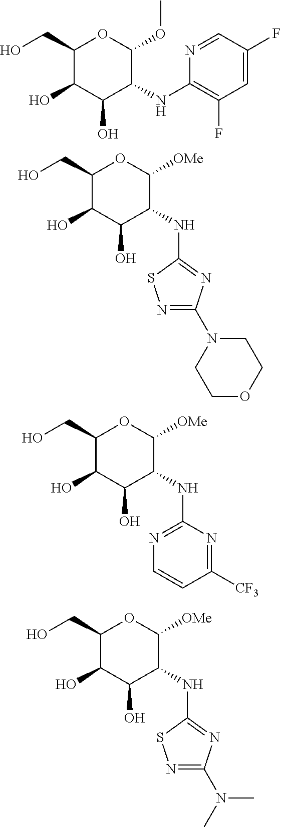

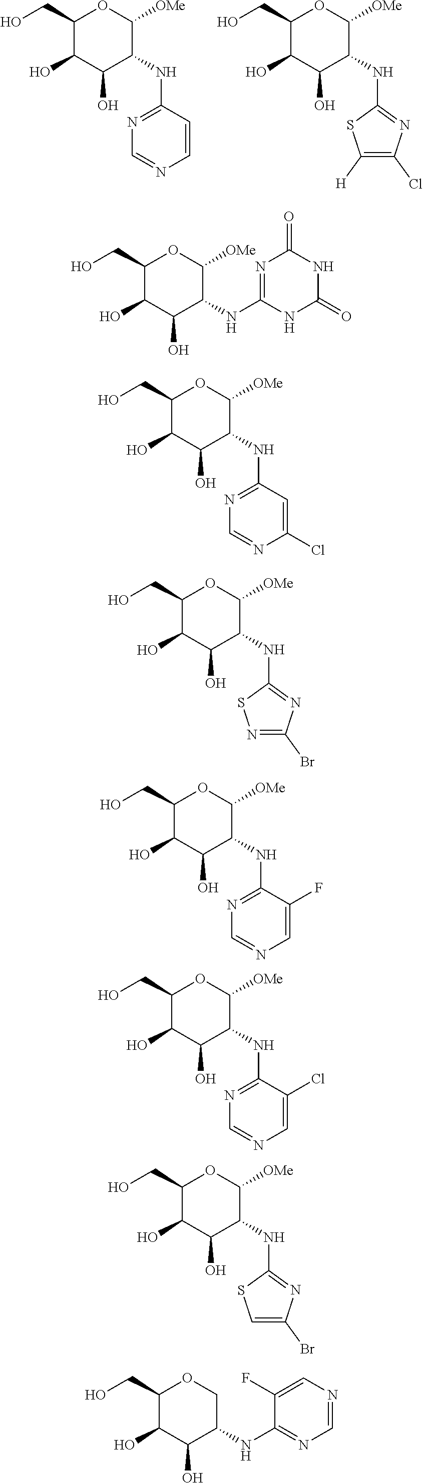

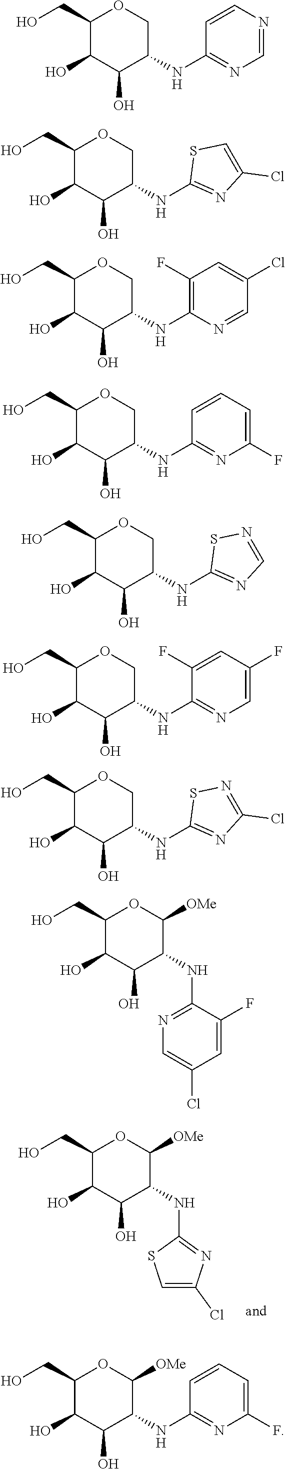

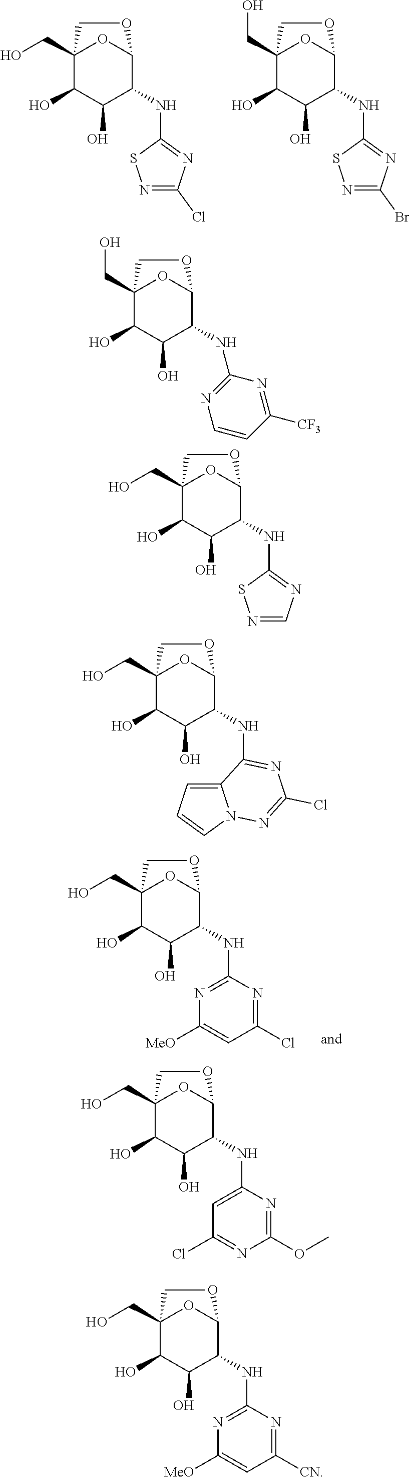

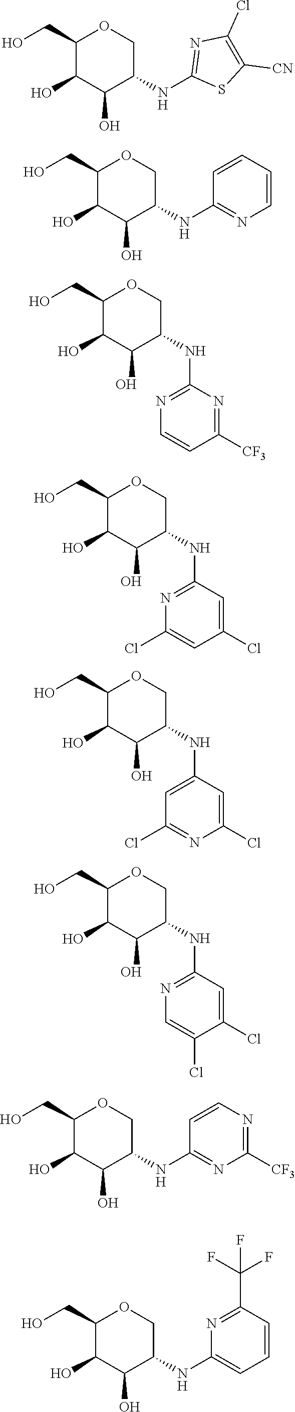

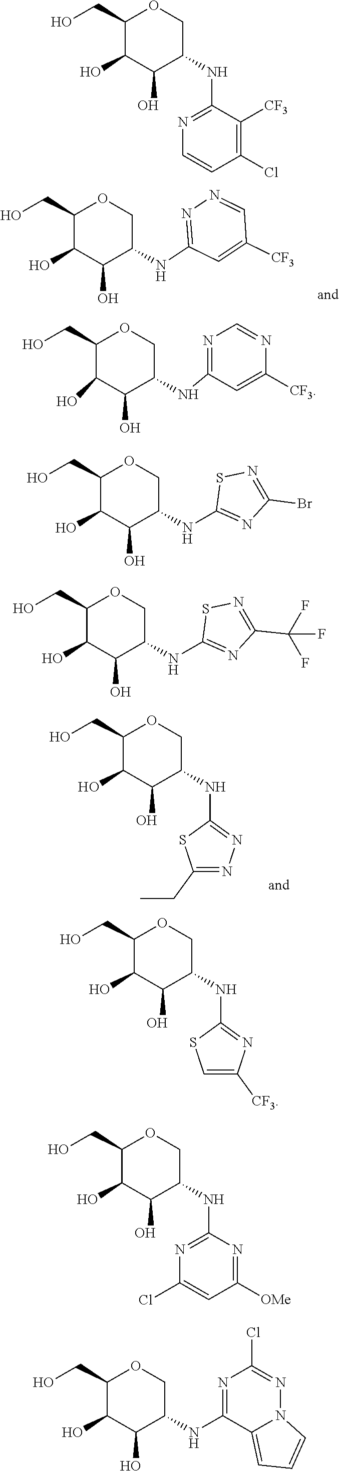

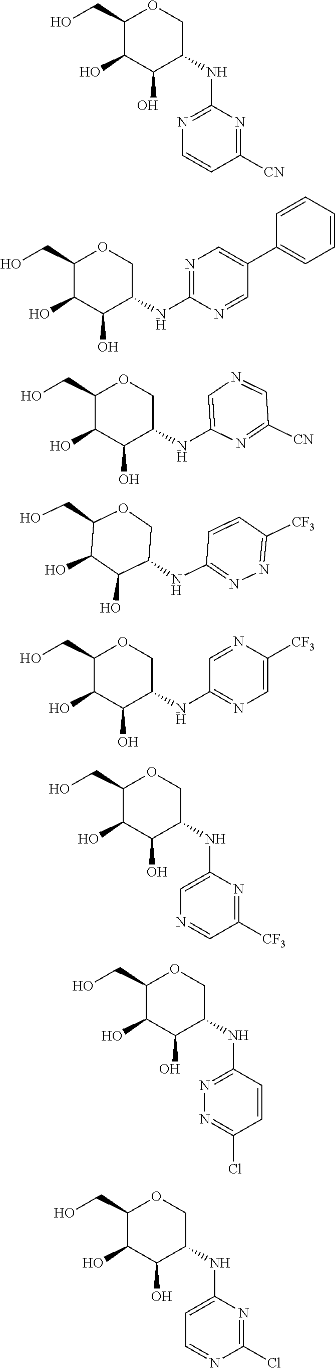

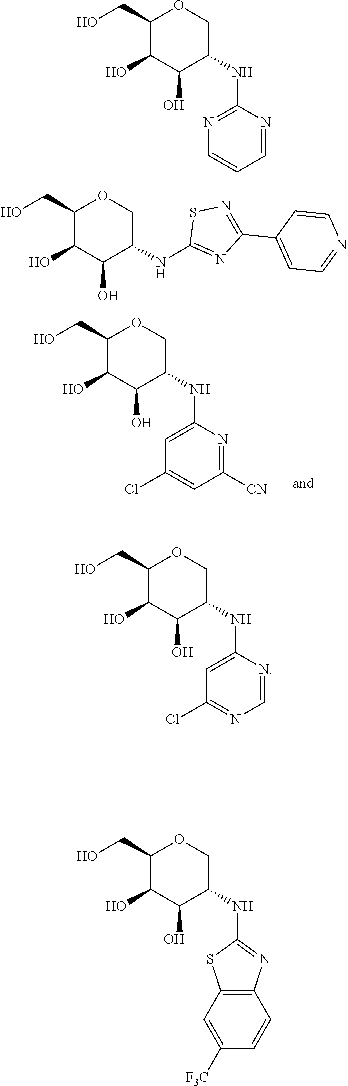

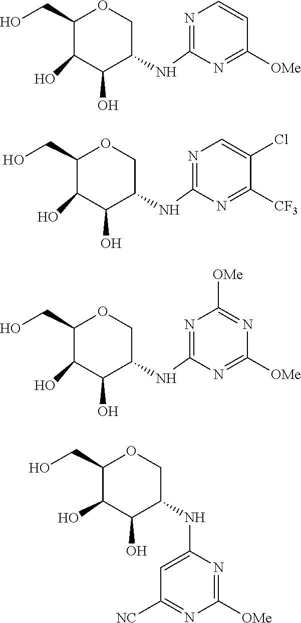

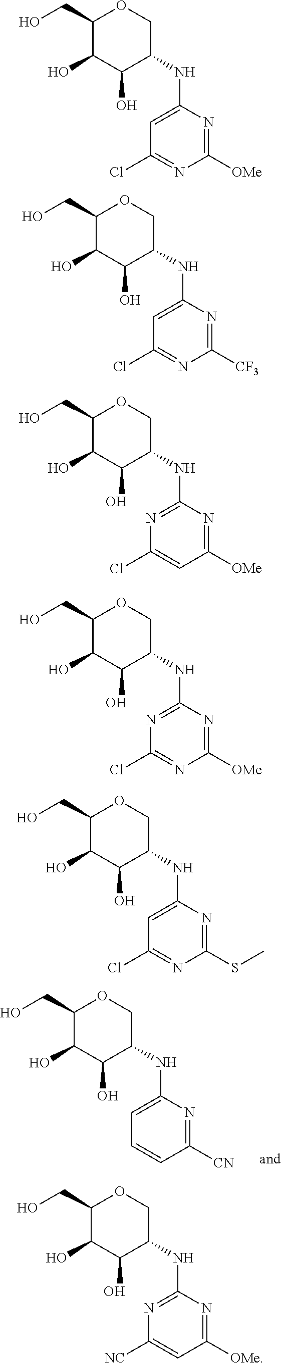

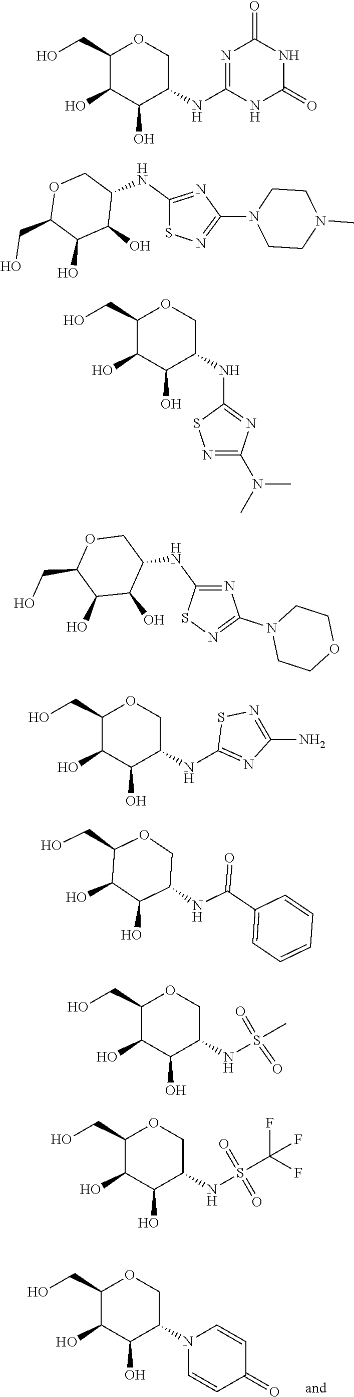

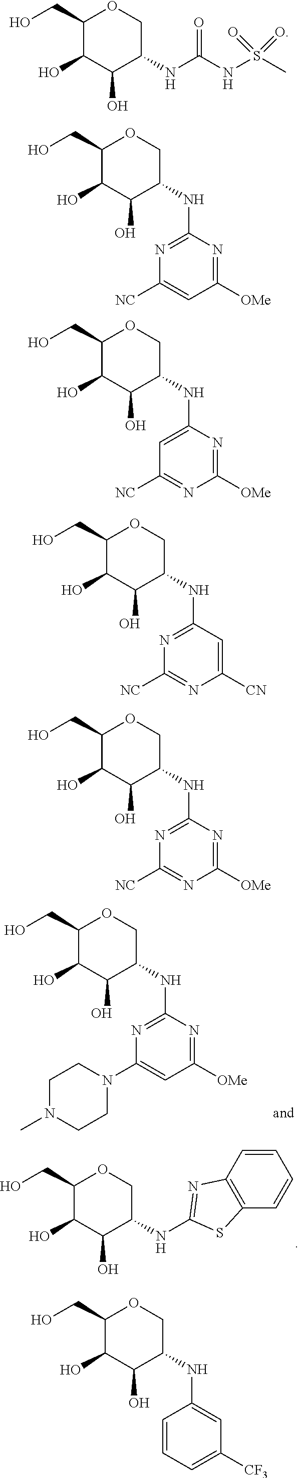

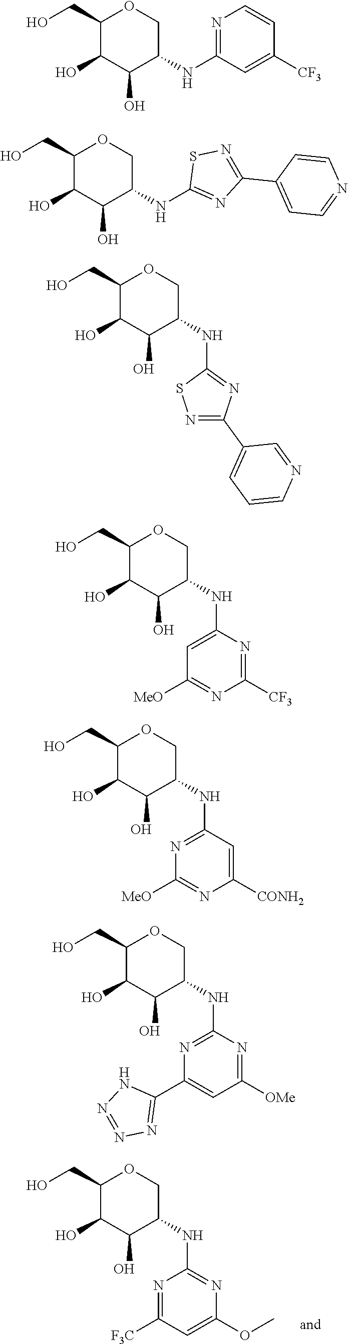

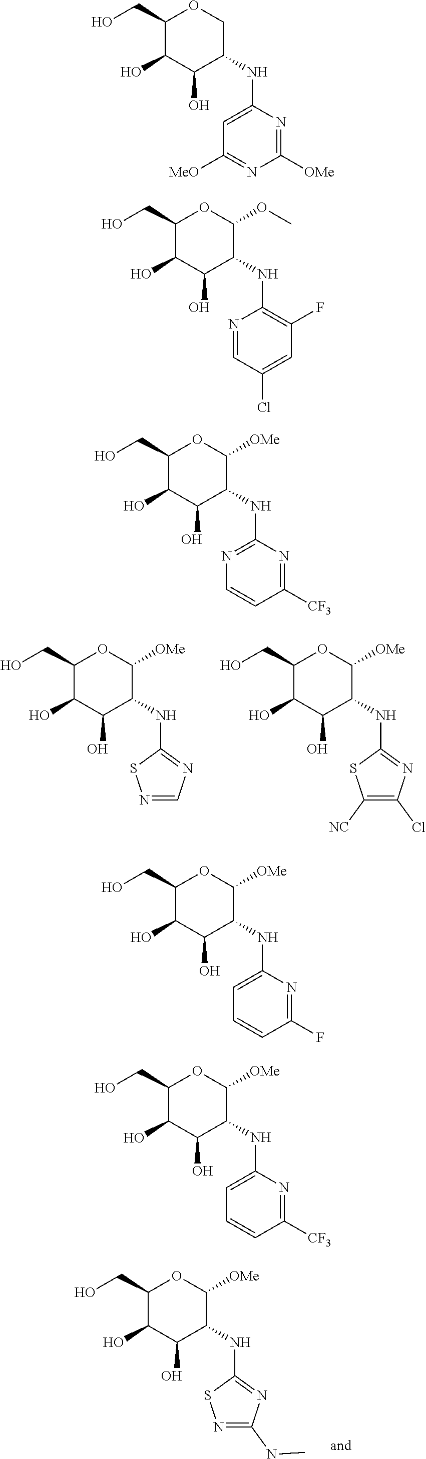

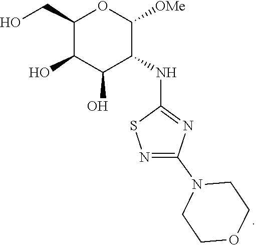 [SMILES: CO[C@H]1O[C@H](CO)[C@H](O)[C@H](O)[C@H]1Nc1nc(N2CCOCC2)ns1]

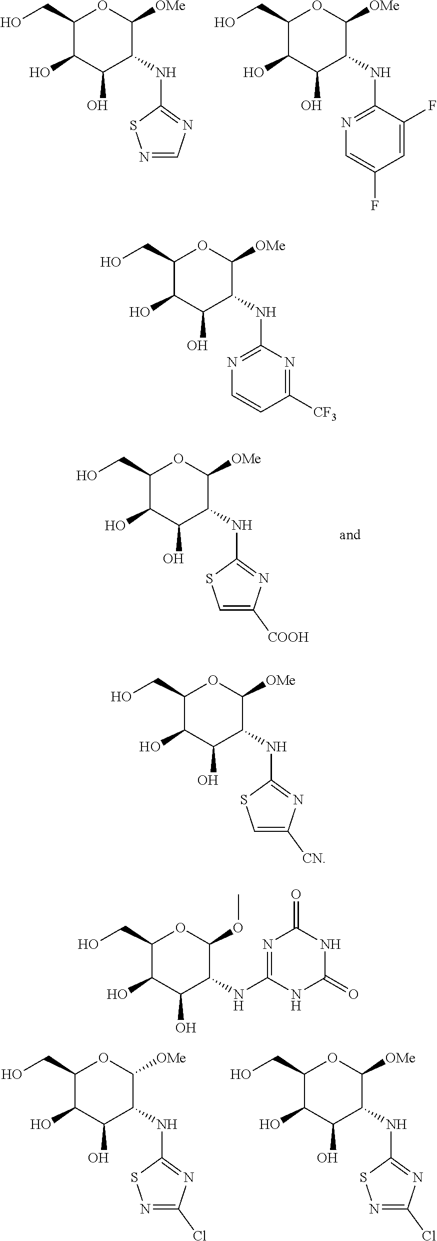

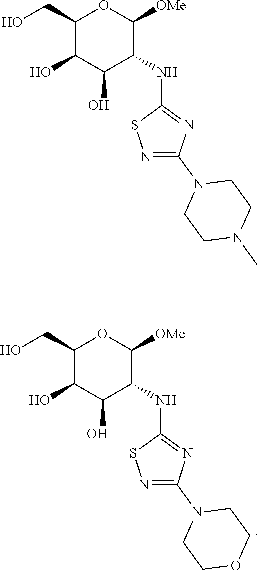 [SMILES: CO[C@@H]1O[C@H](CO)[C@H](O)[C@H](O)[C@H]1Nc1nc(N2CCN(C)CC2)ns1 ; CO[C@@H]1O[C@H](CO)[C@H](O)[C@H](O)[C@H]1Nc1nc(N2CCOCC2)ns1]

In certain embodiments, the compound of Formula II is selected from:

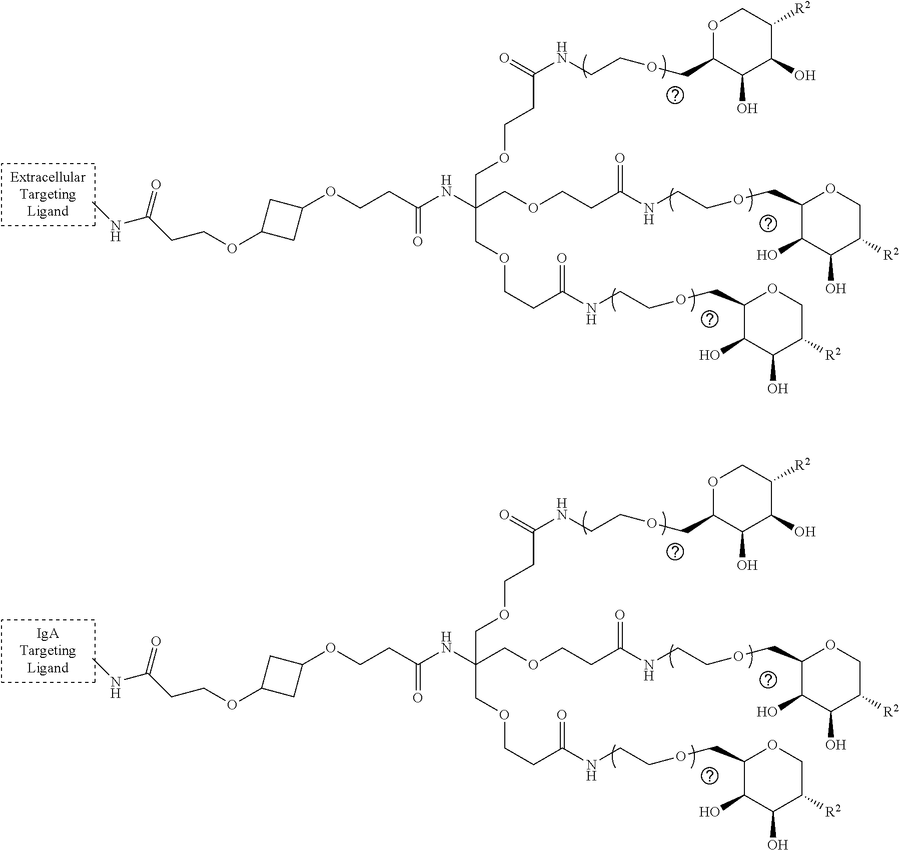

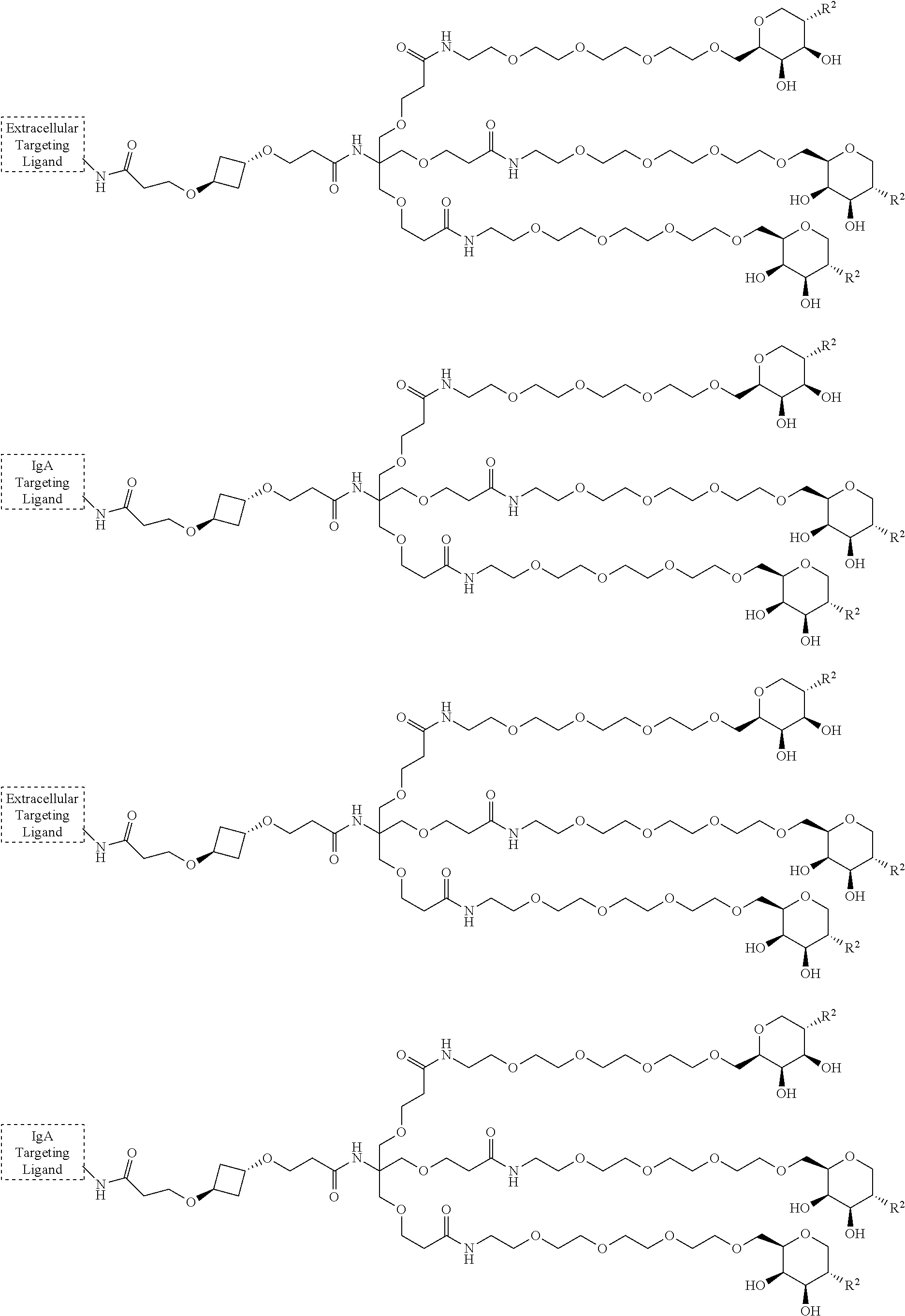

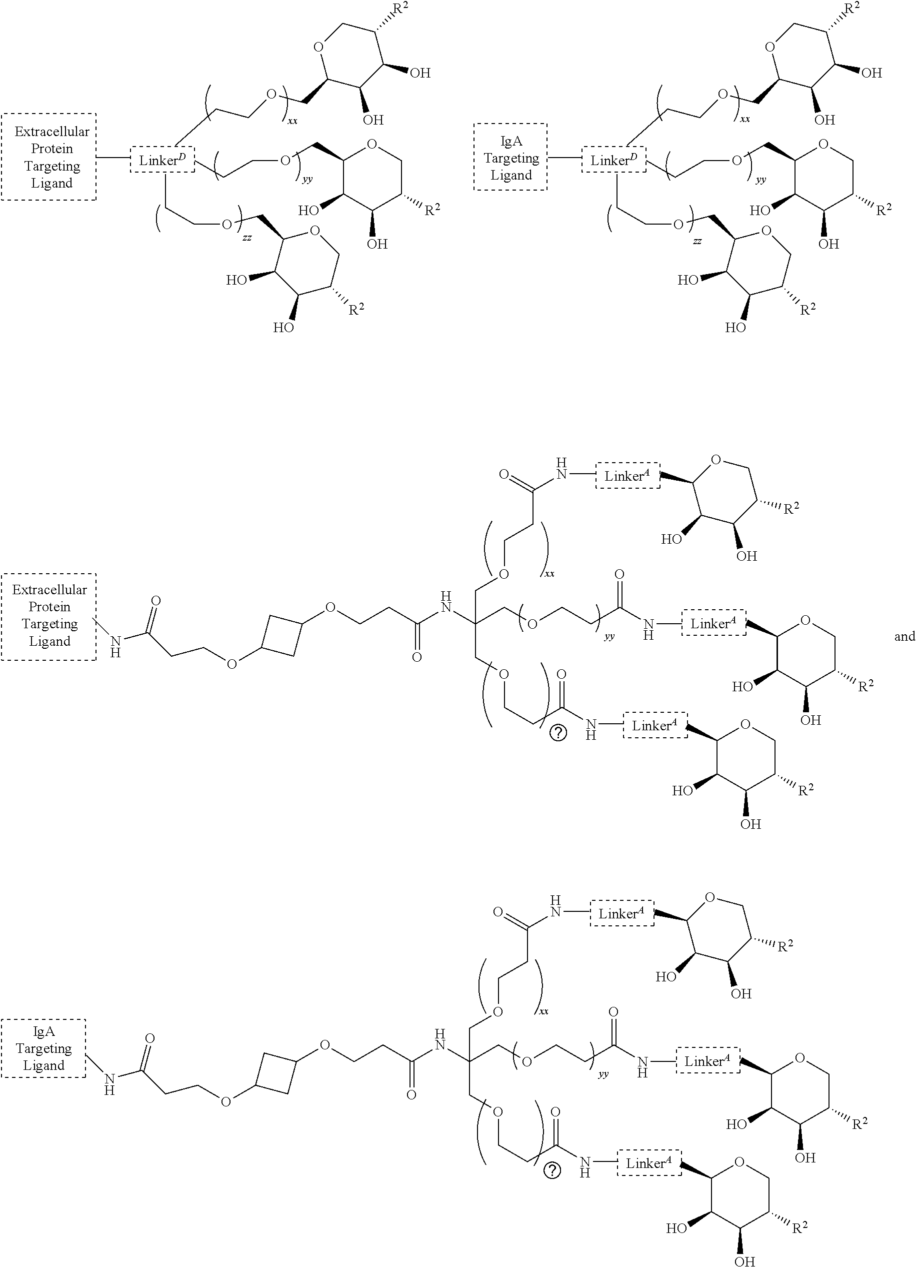

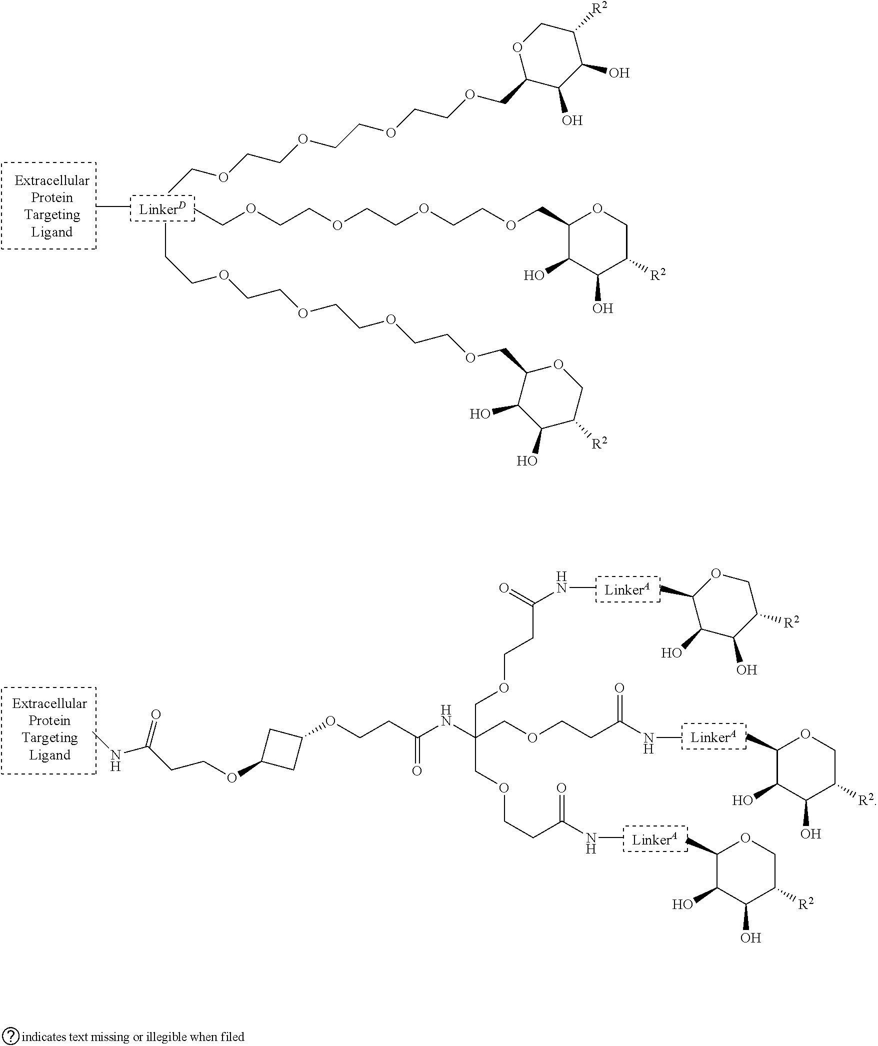

### B. Talose-Based ASGPR-Binding Cellular Receptor Binding Moieties of Formula II

In certain embodiments, the compound of Formula II is selected from:

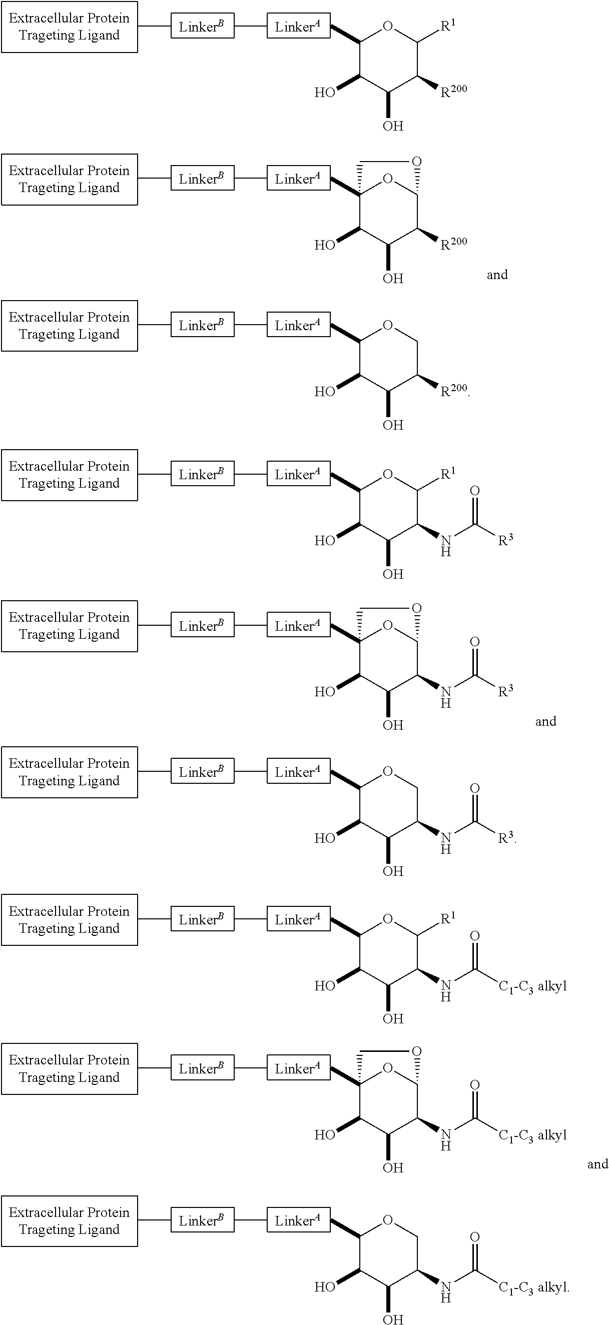

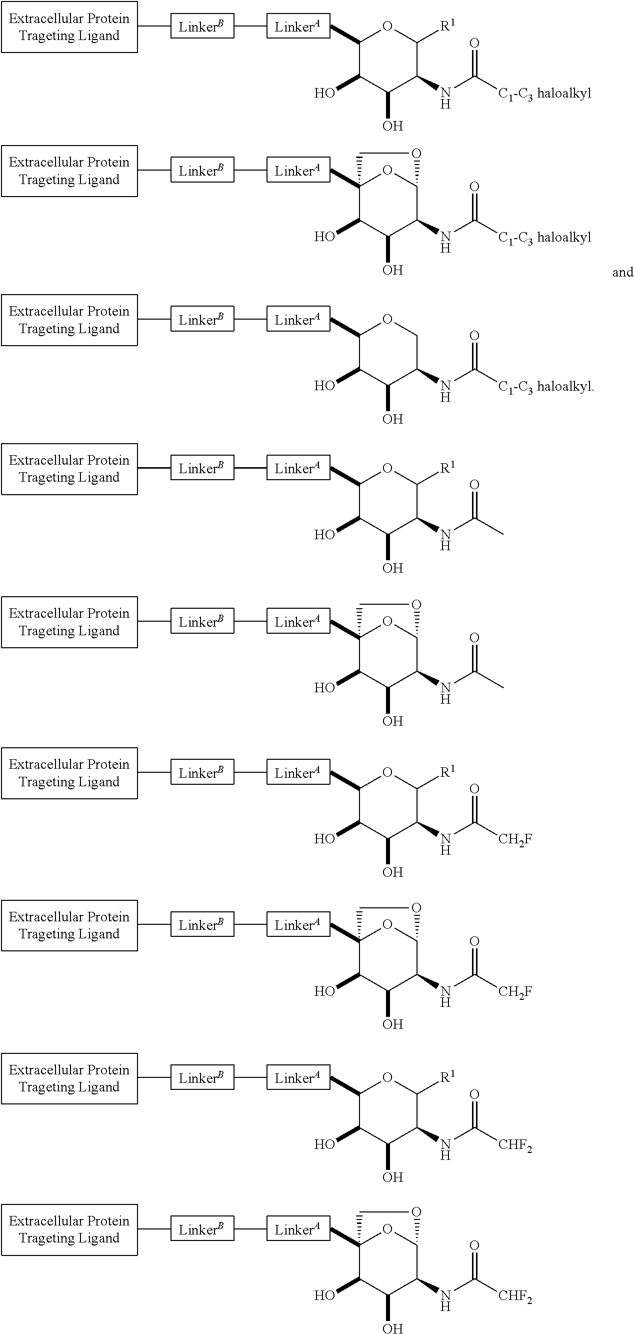

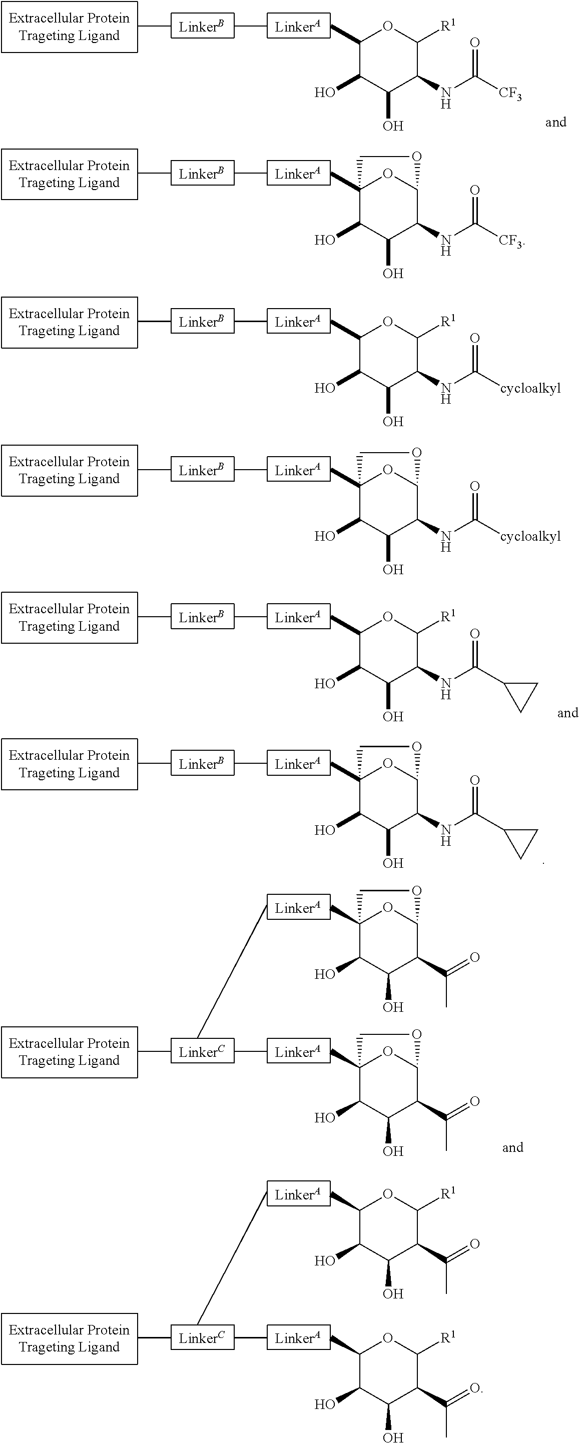

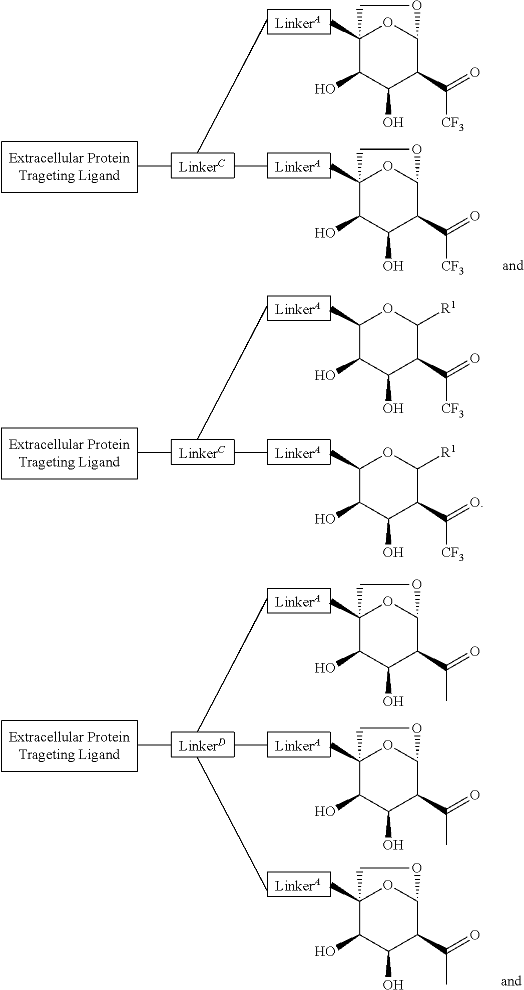

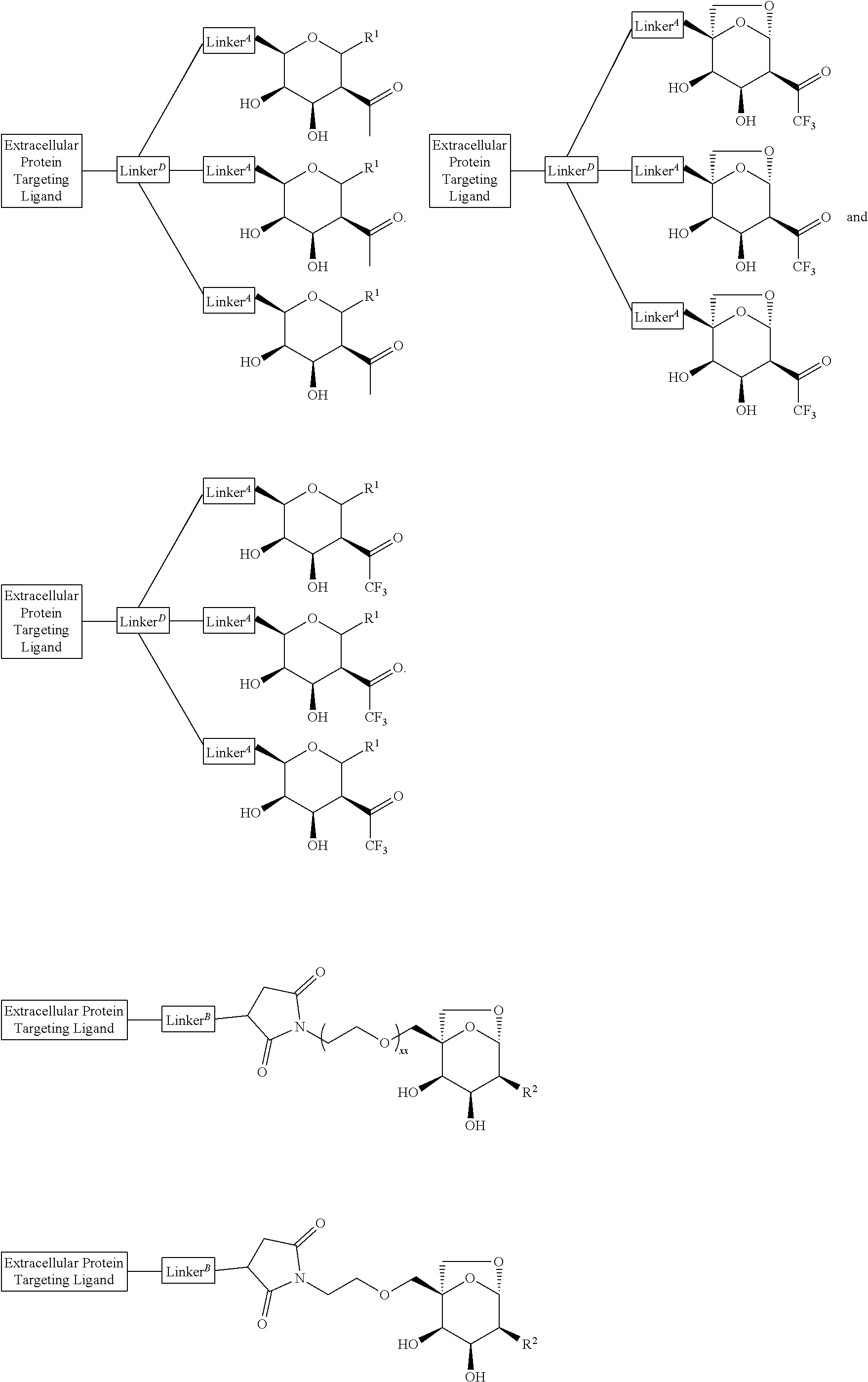

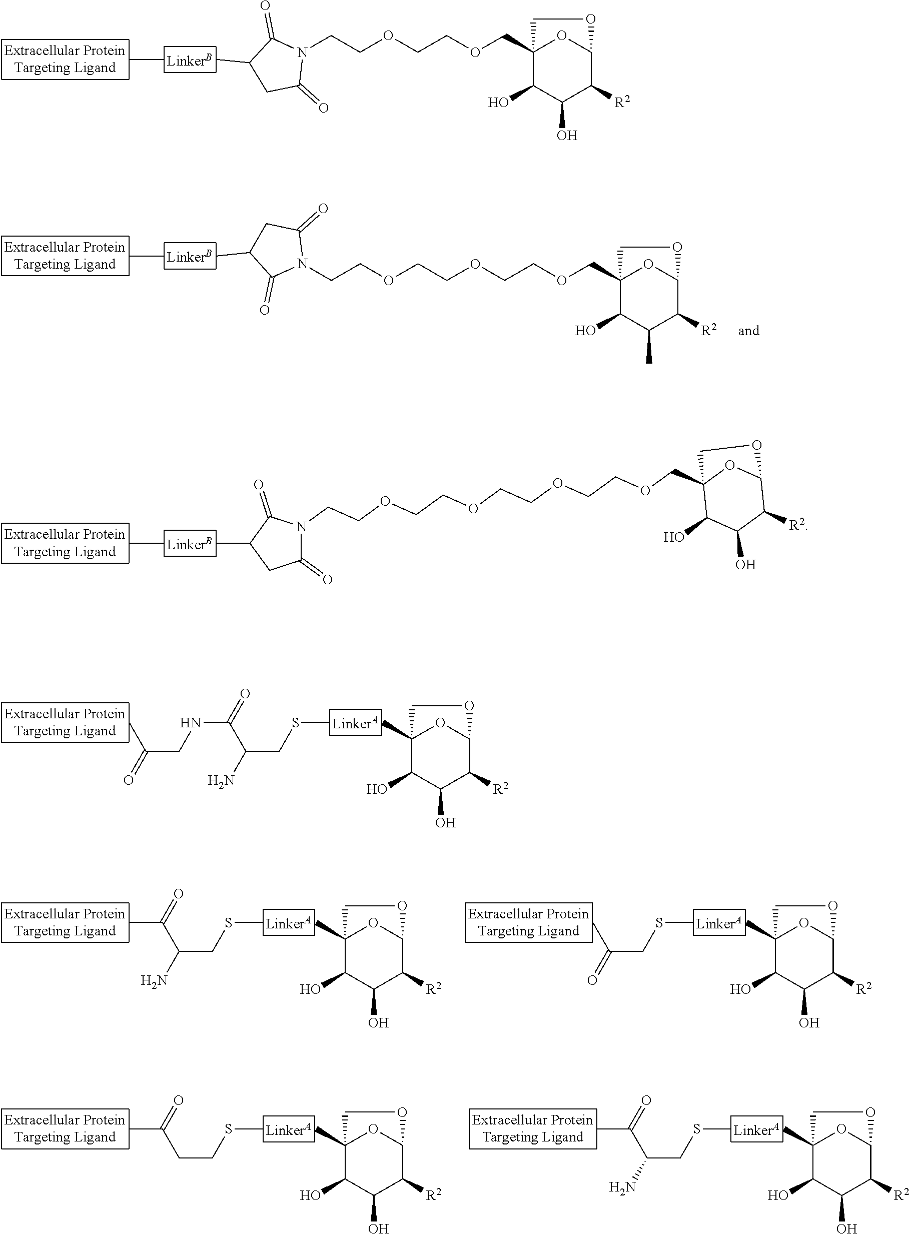

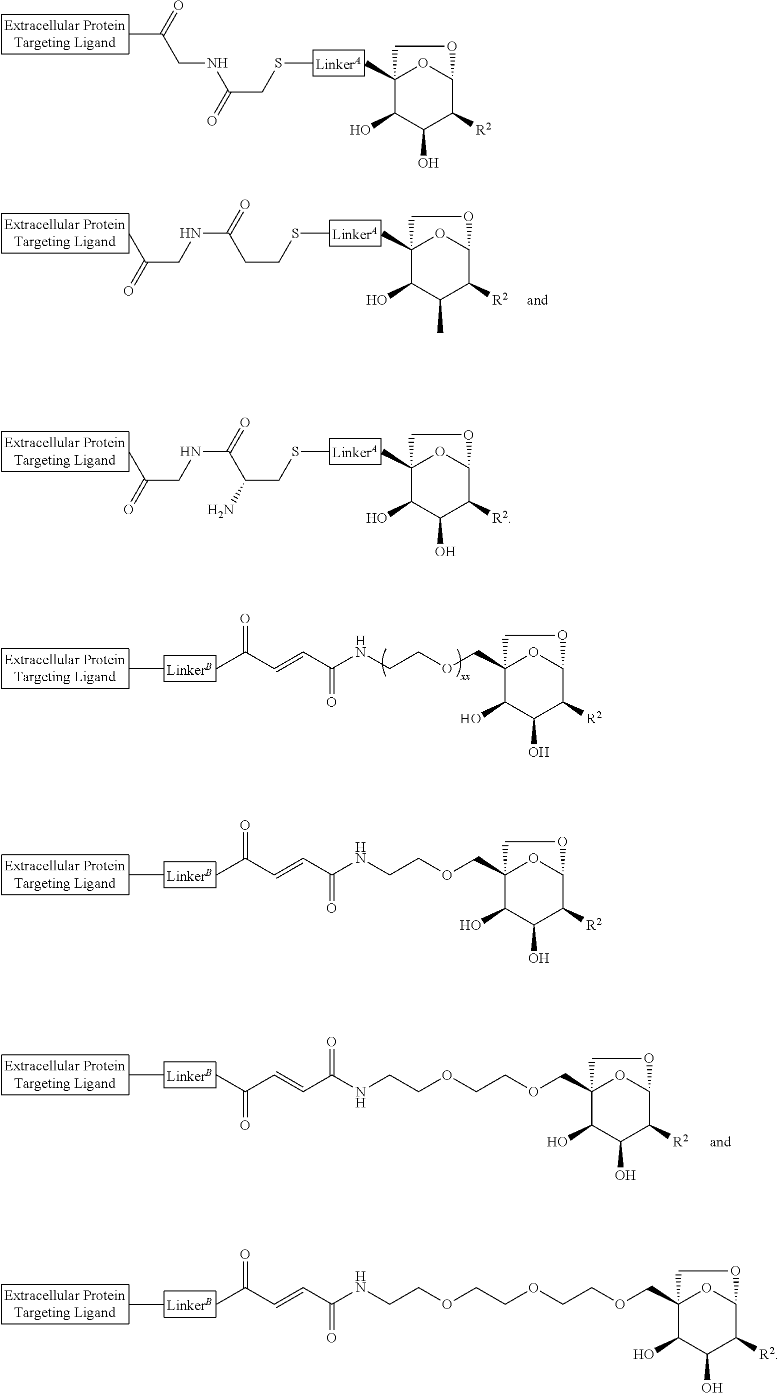

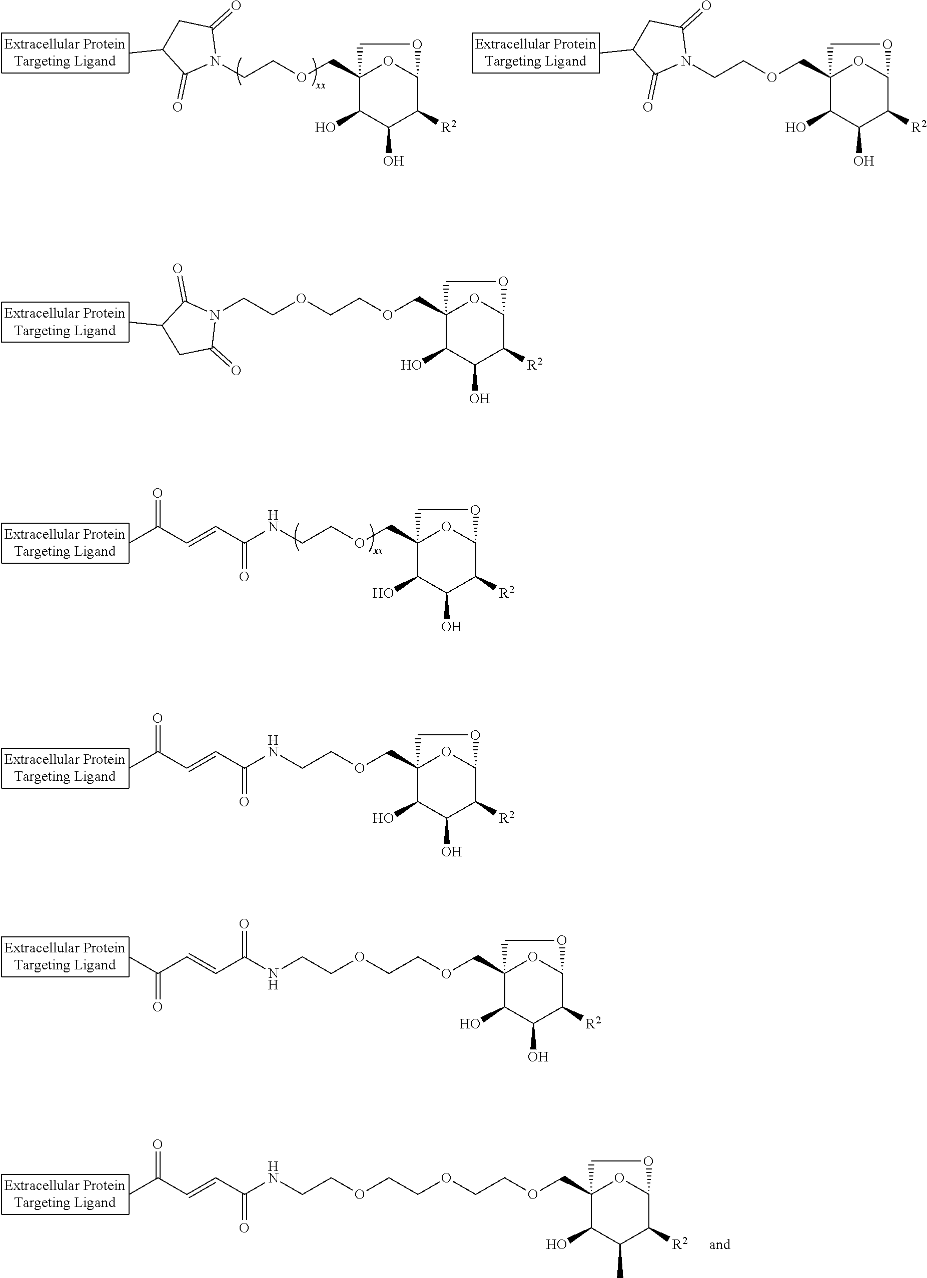

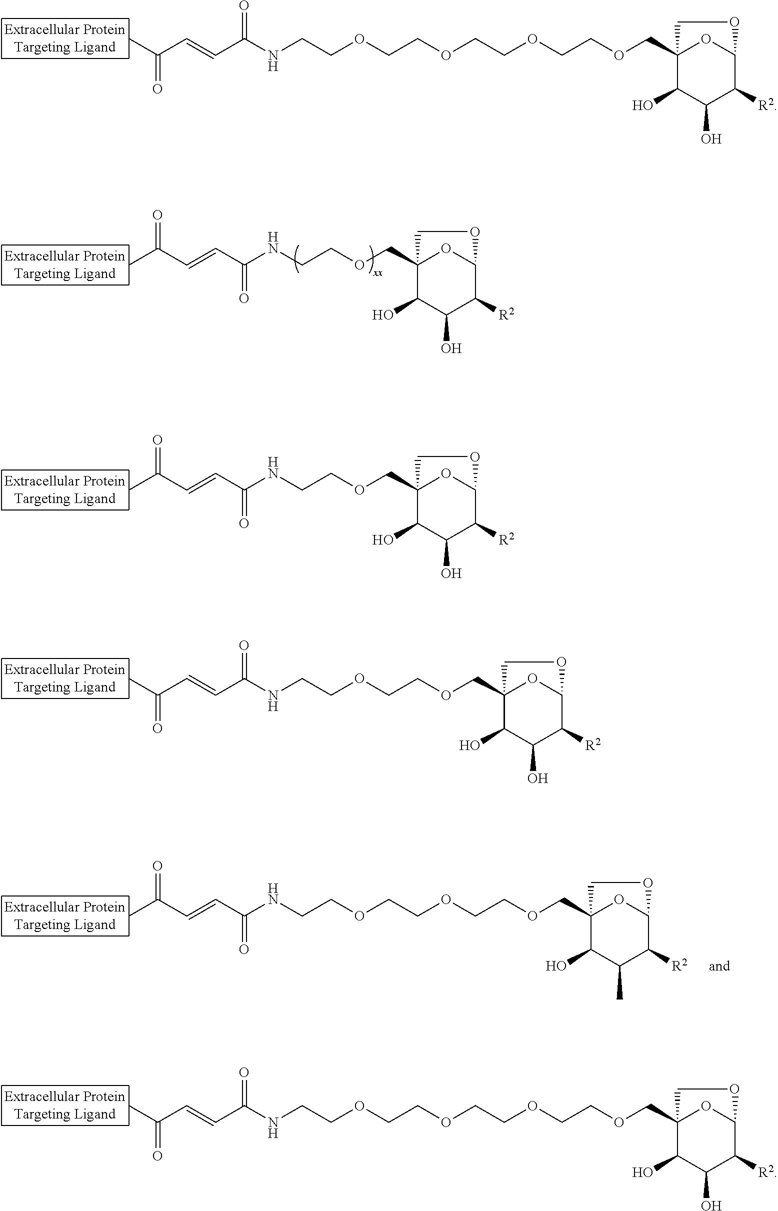

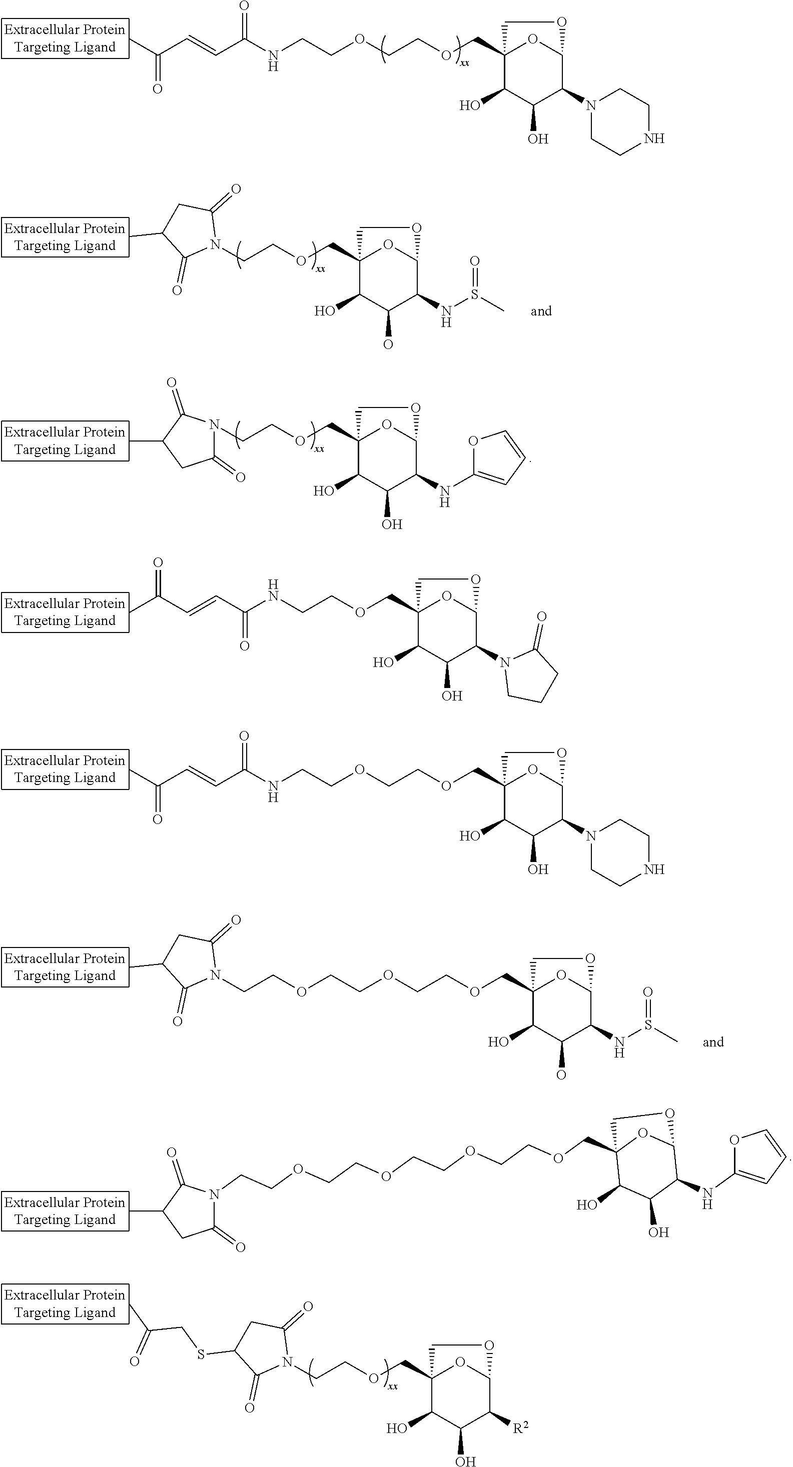

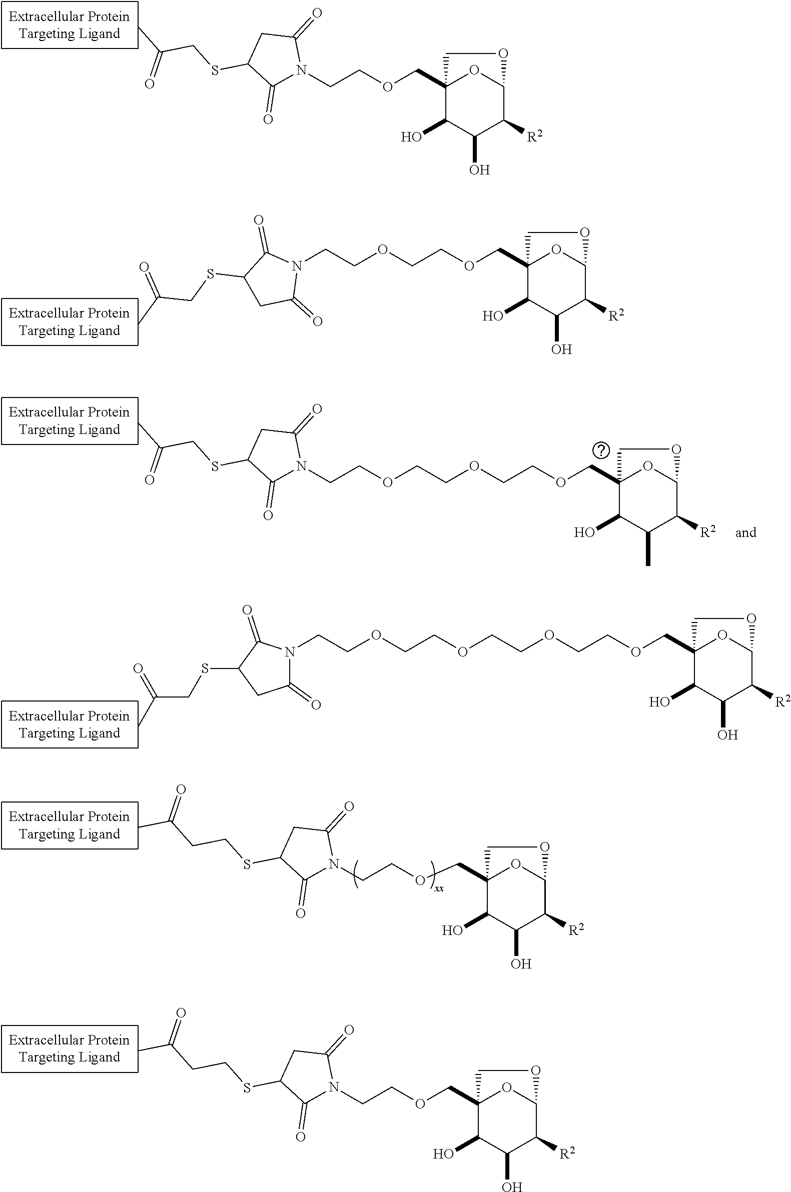

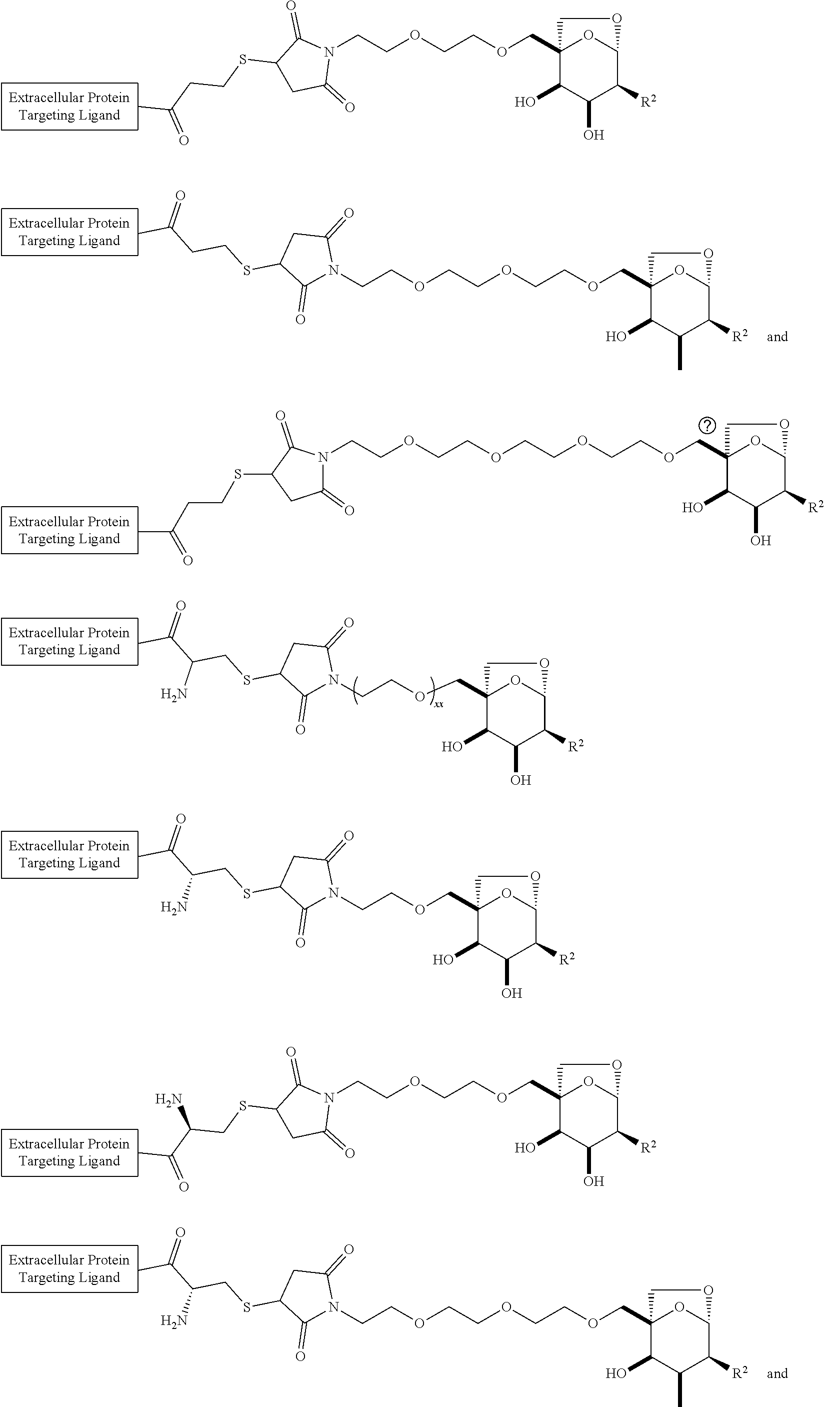

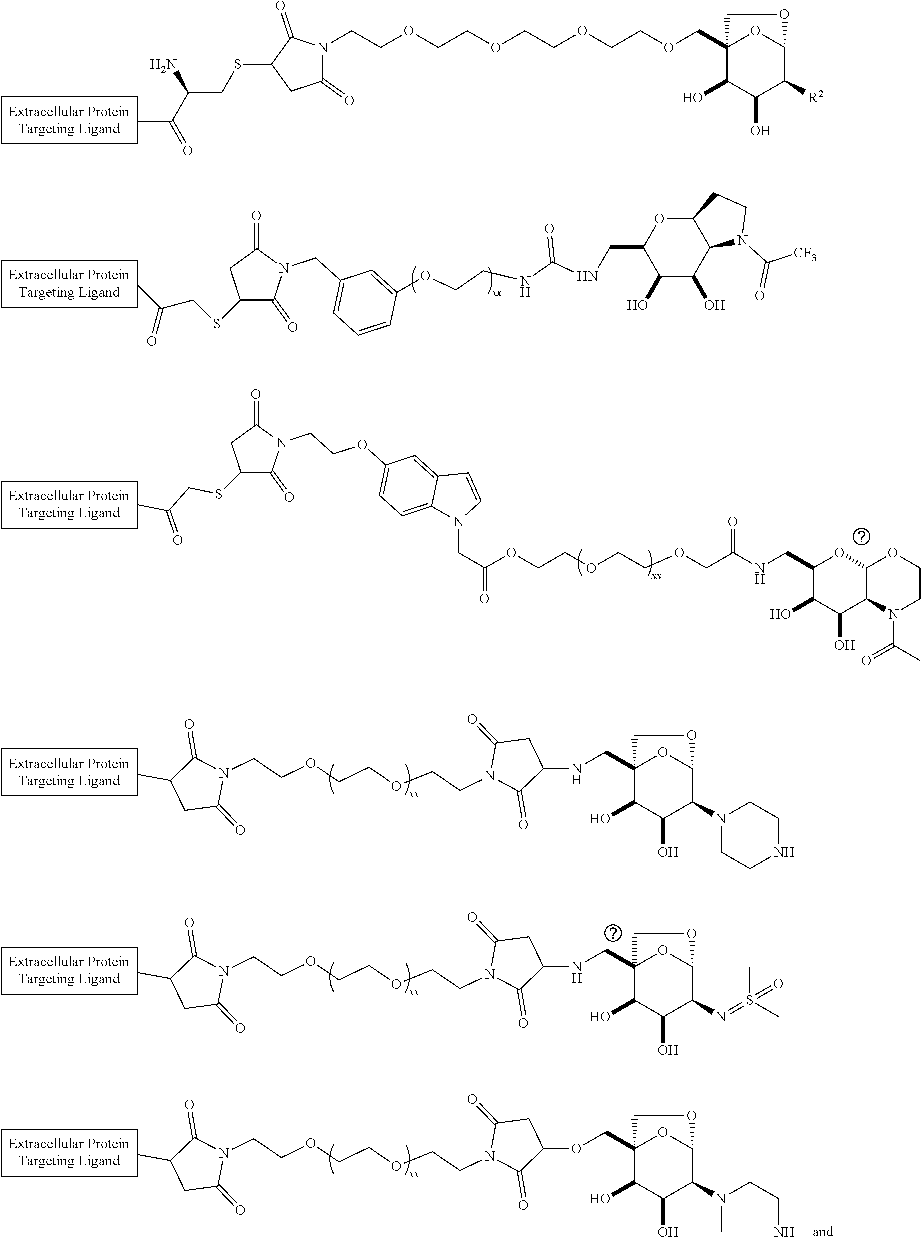

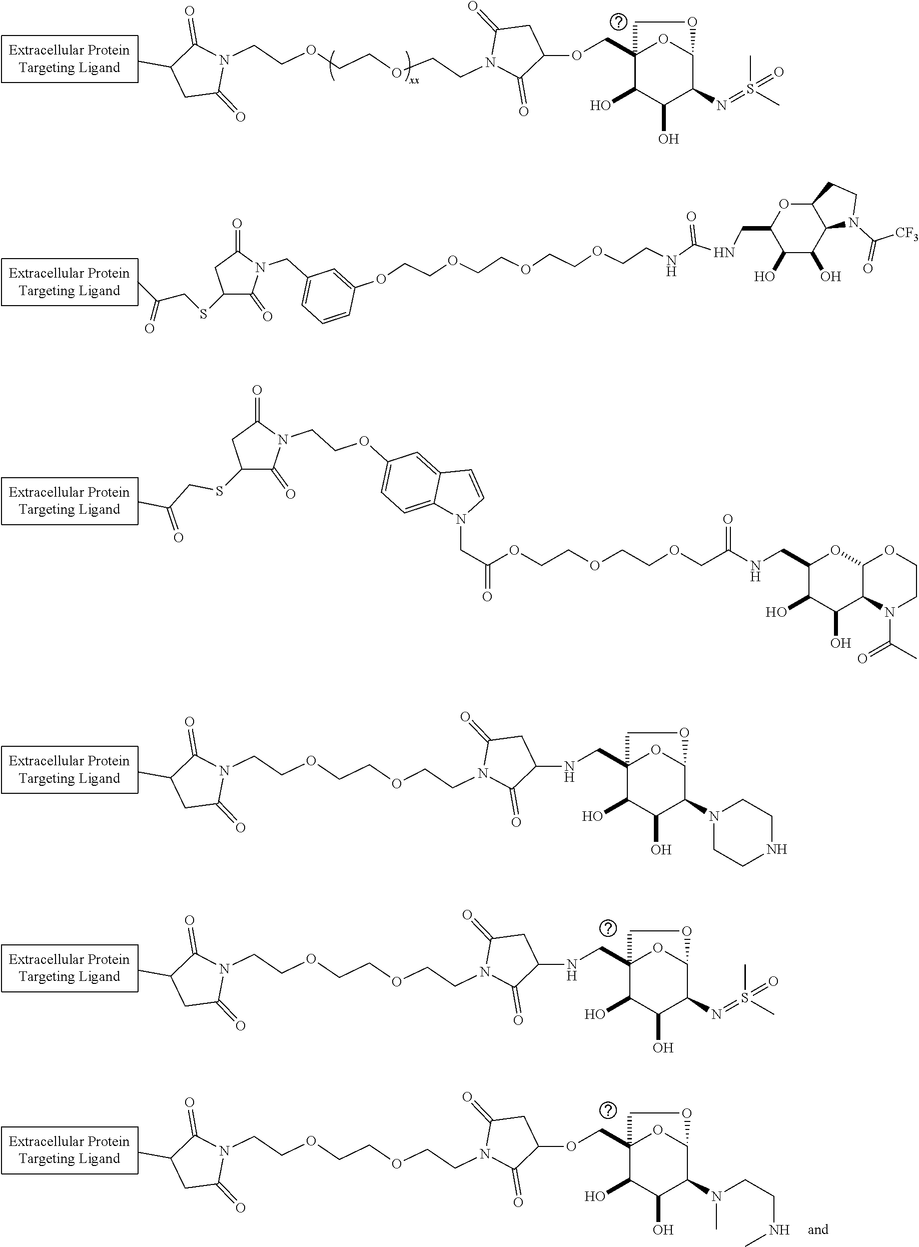

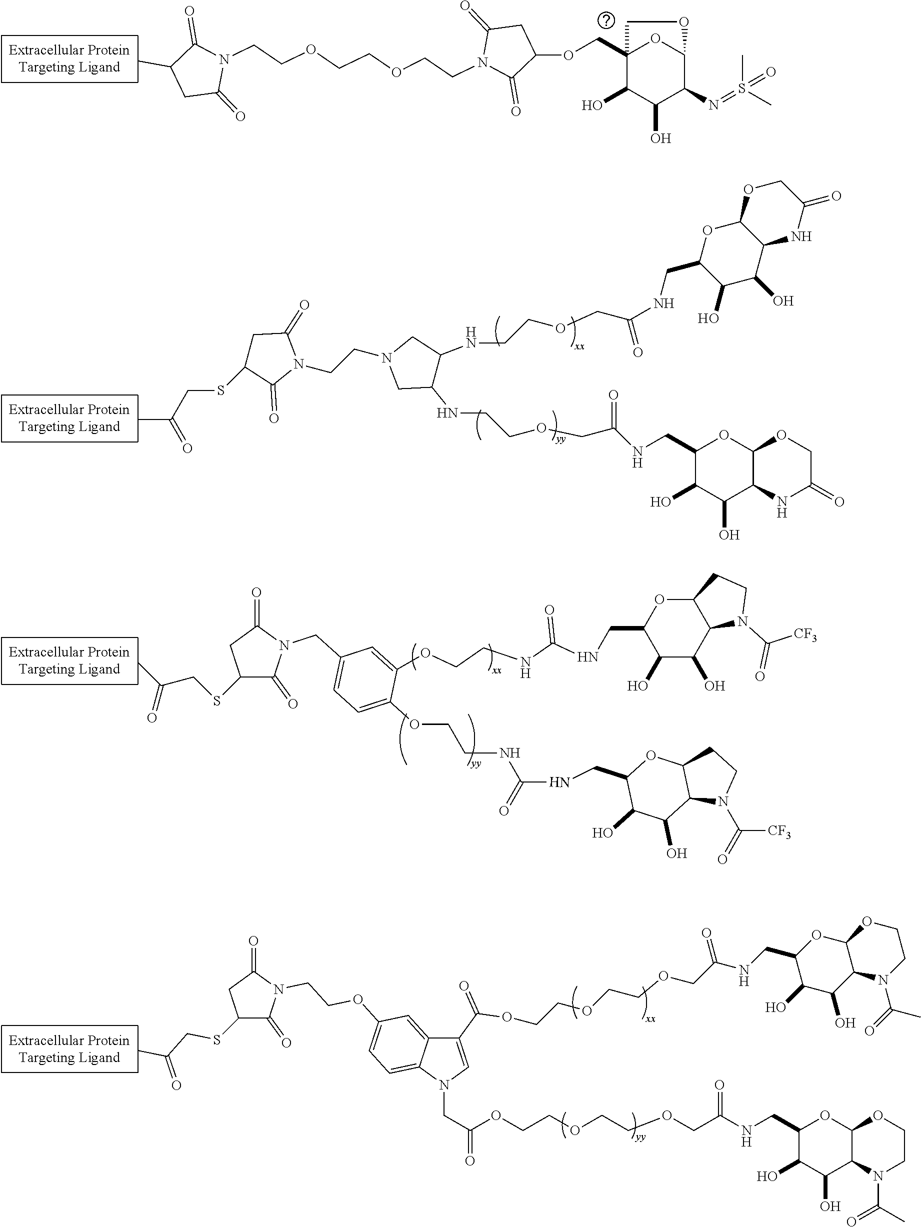

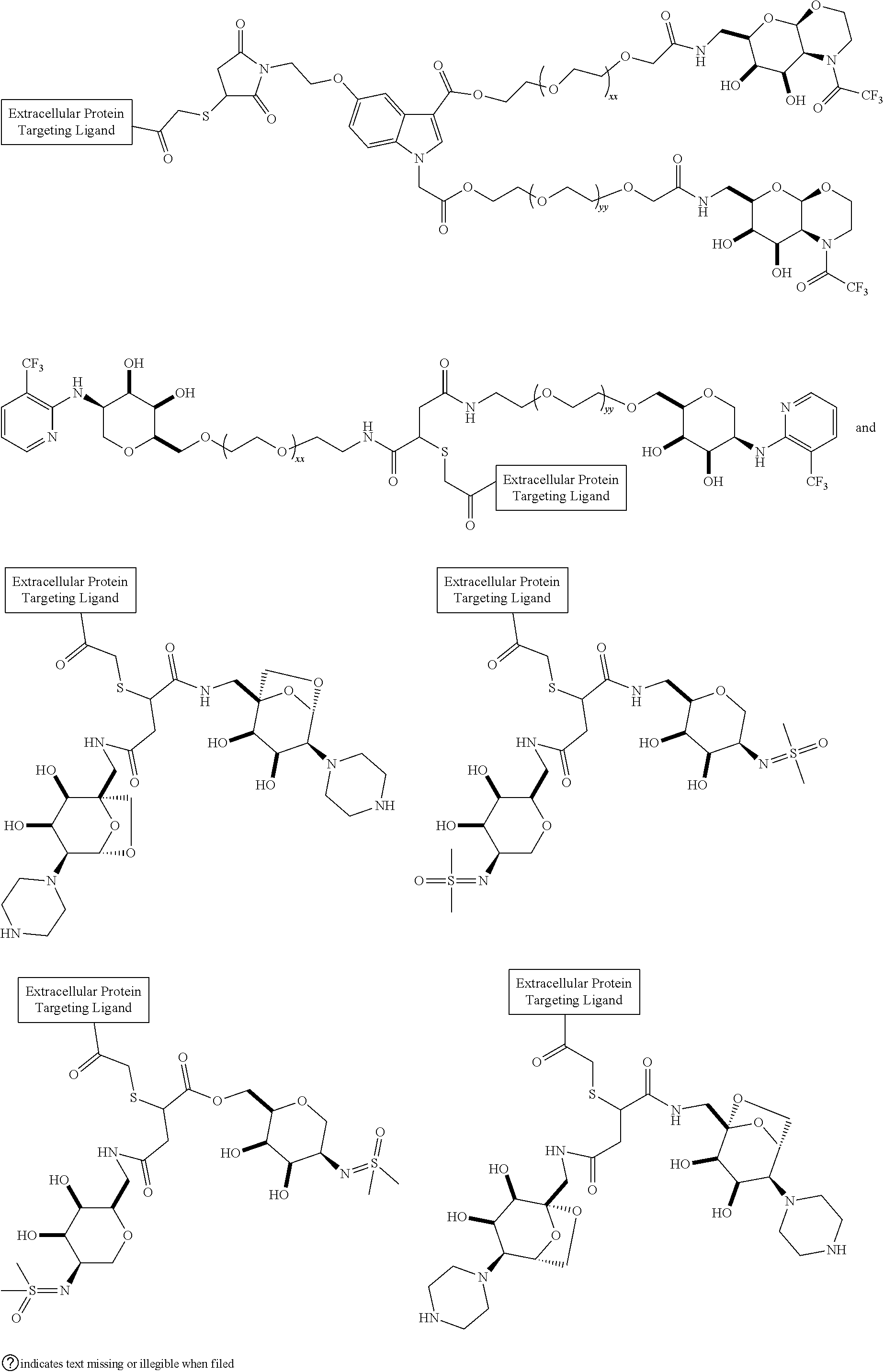

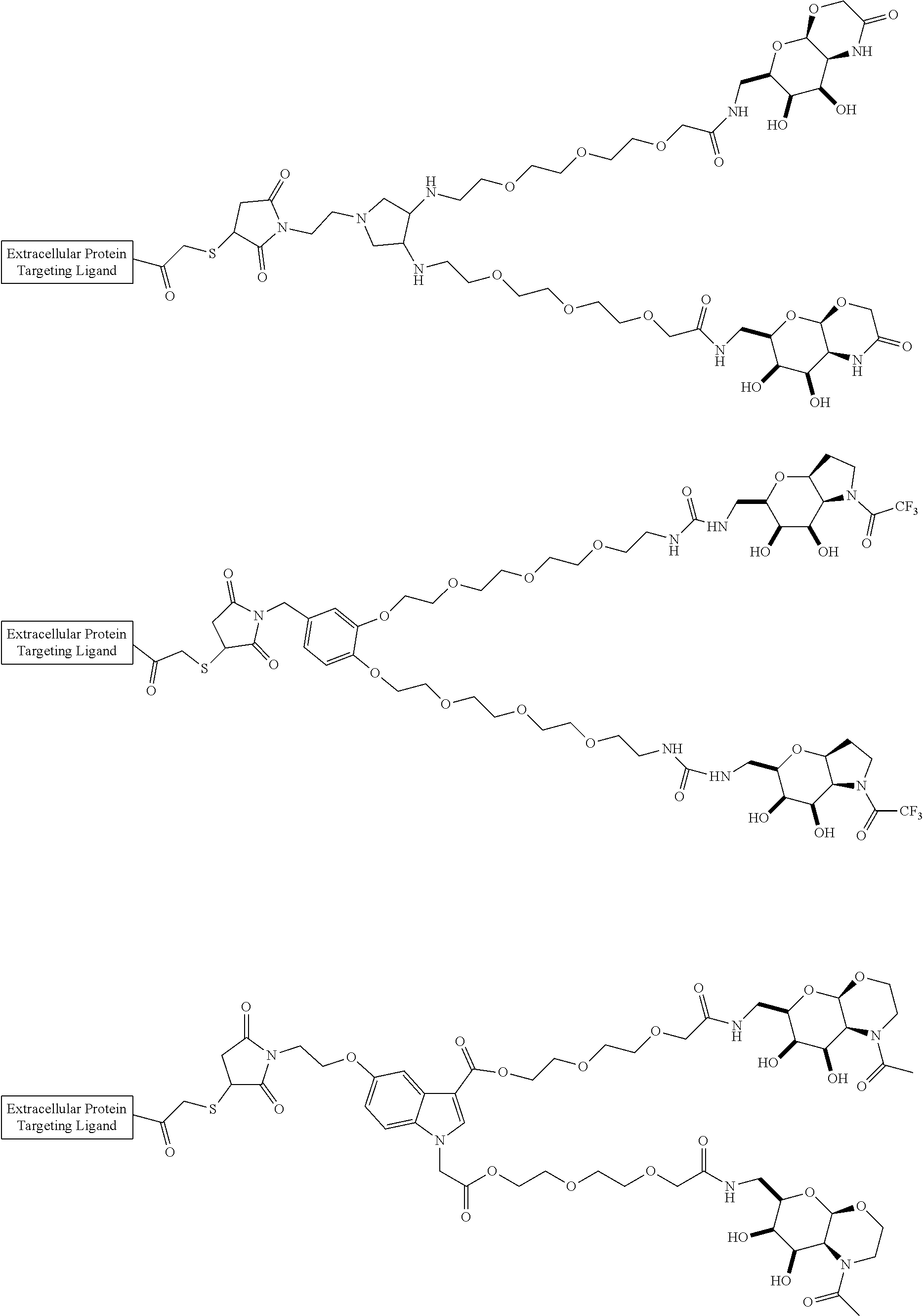

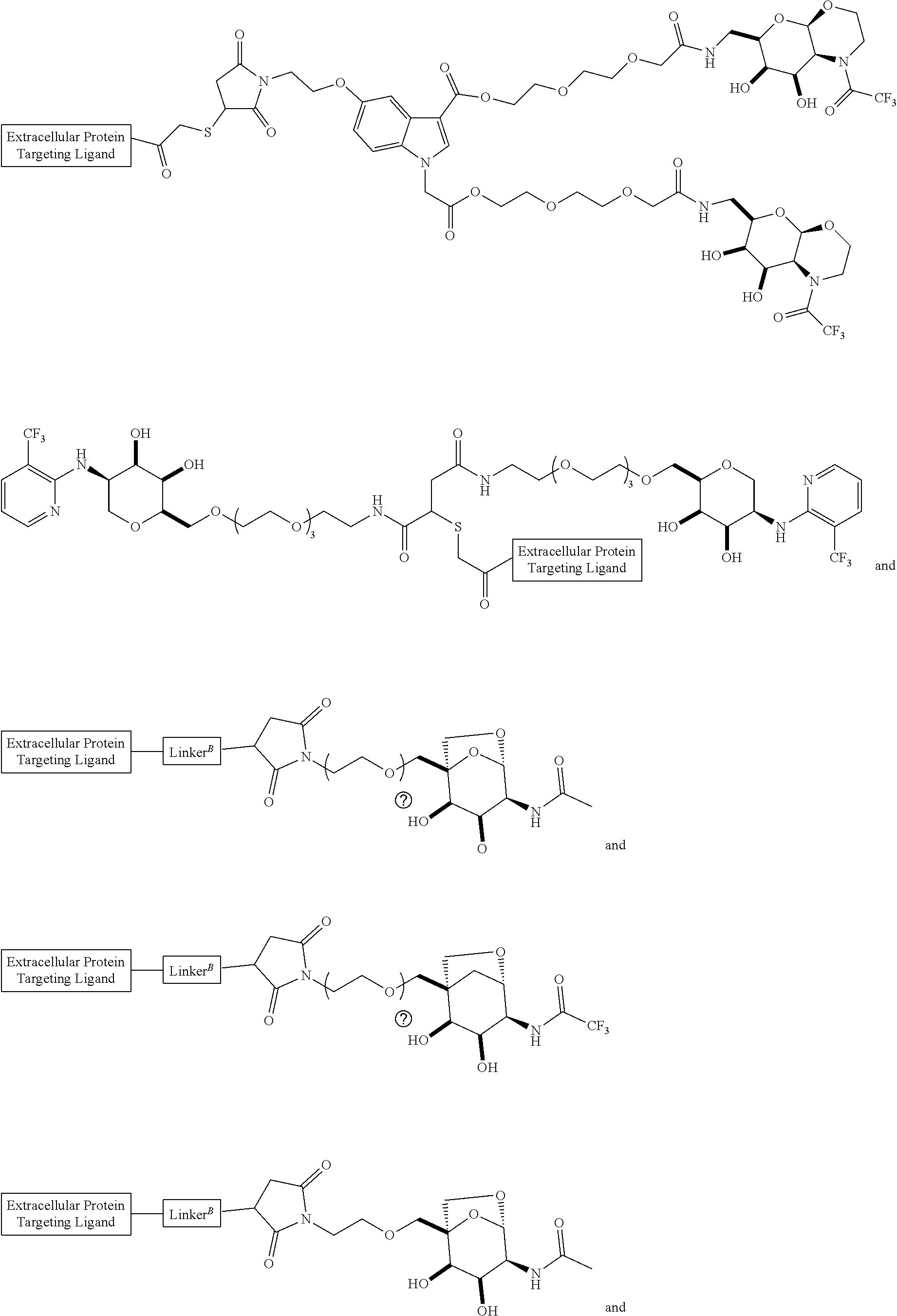

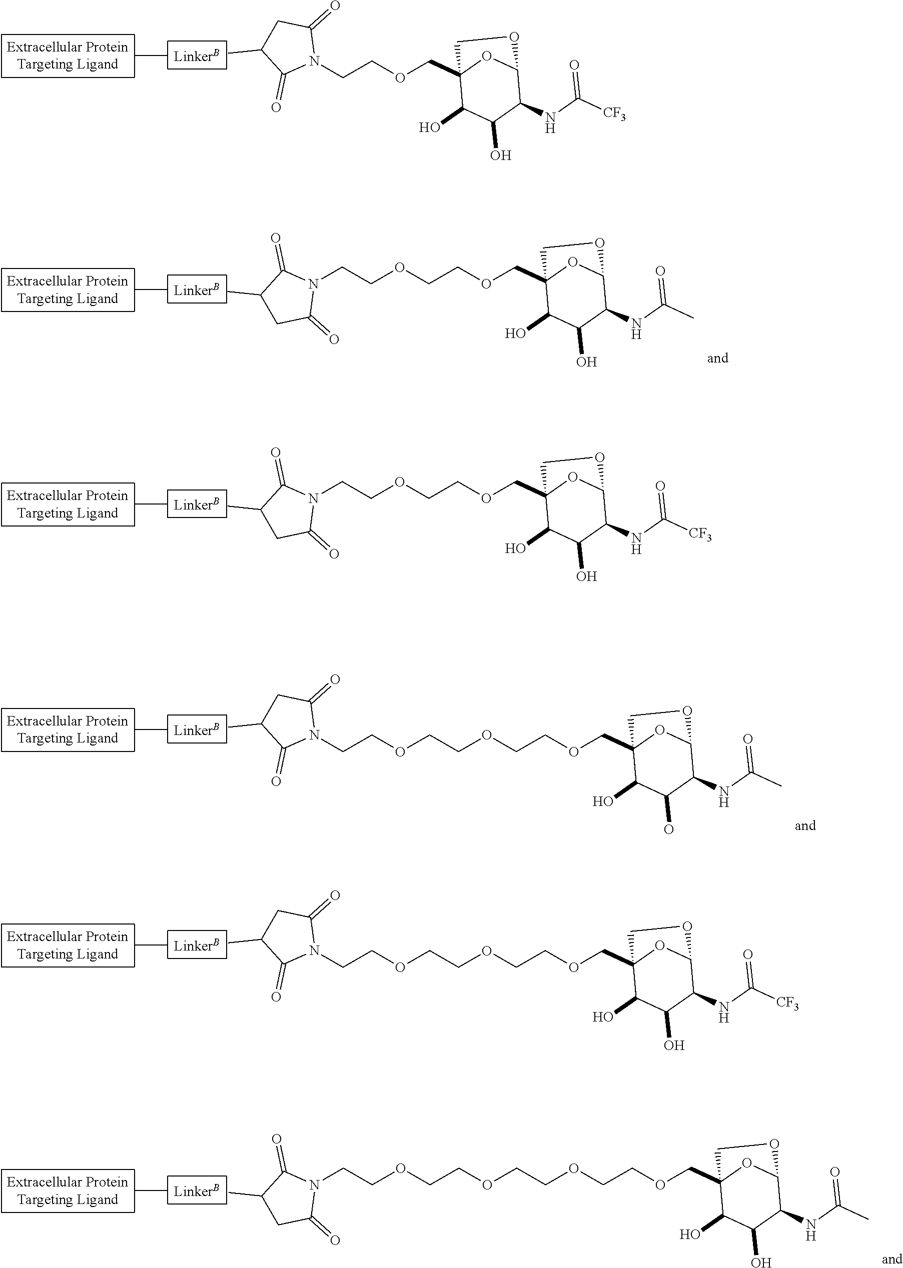

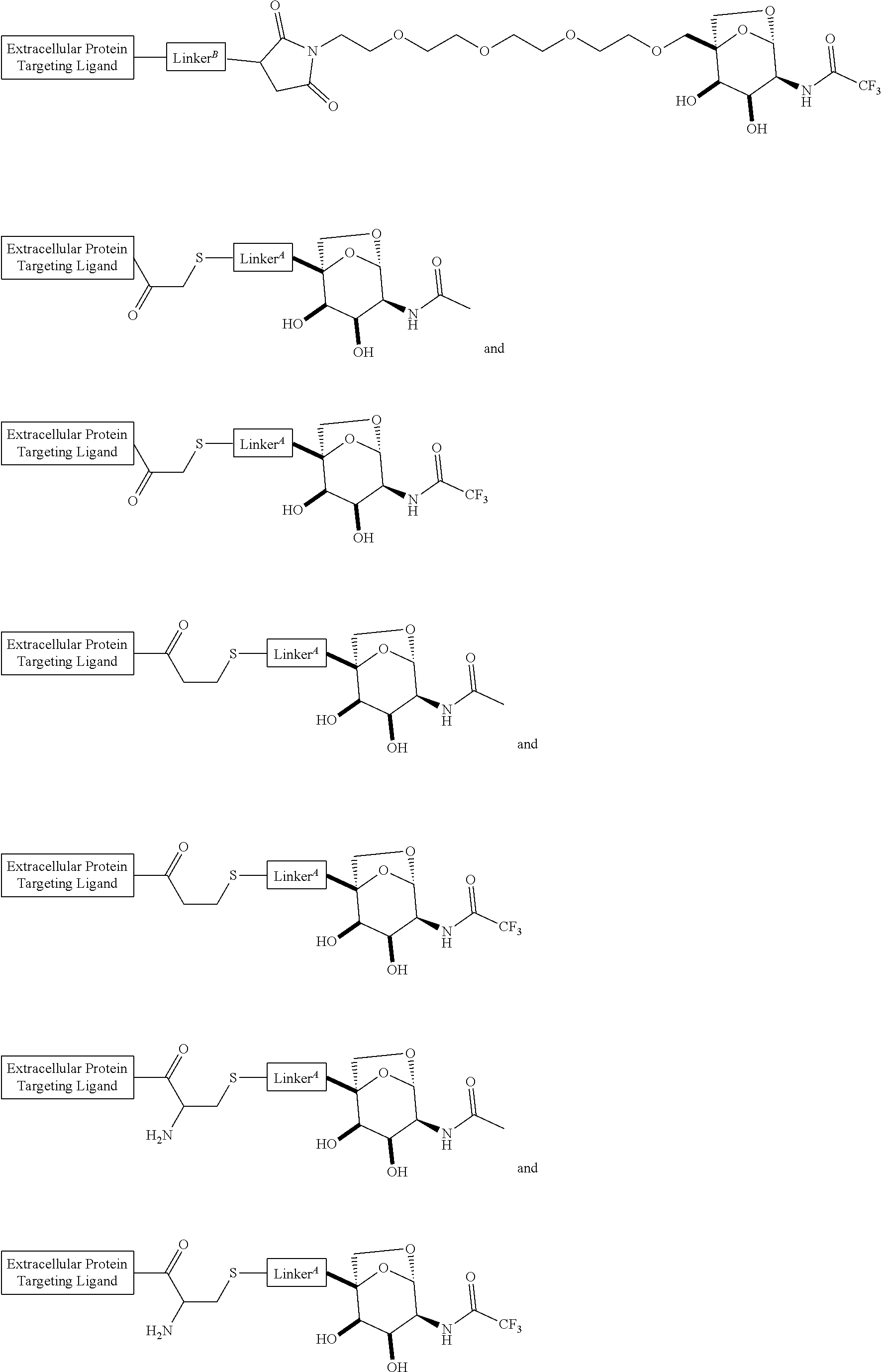

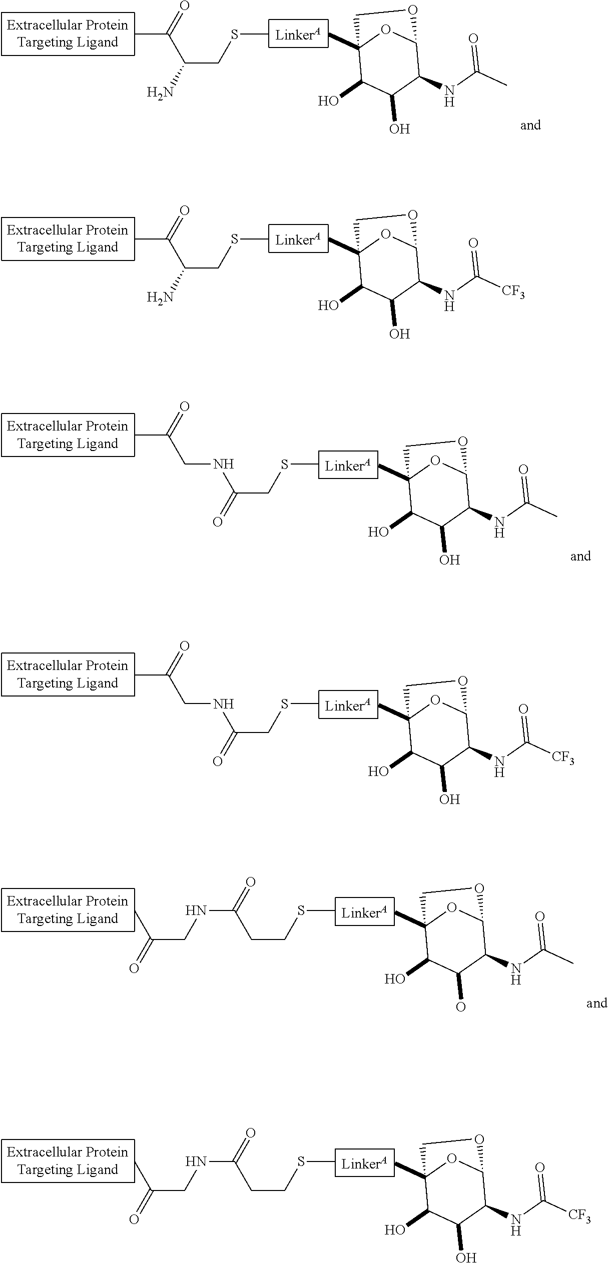

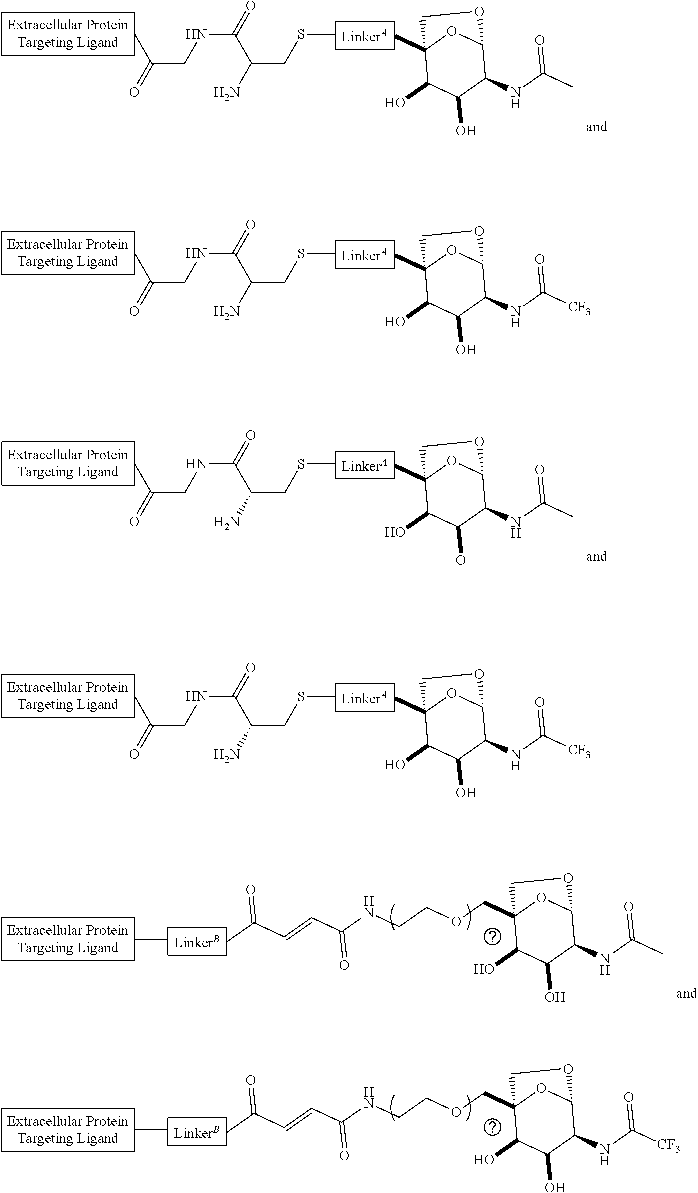

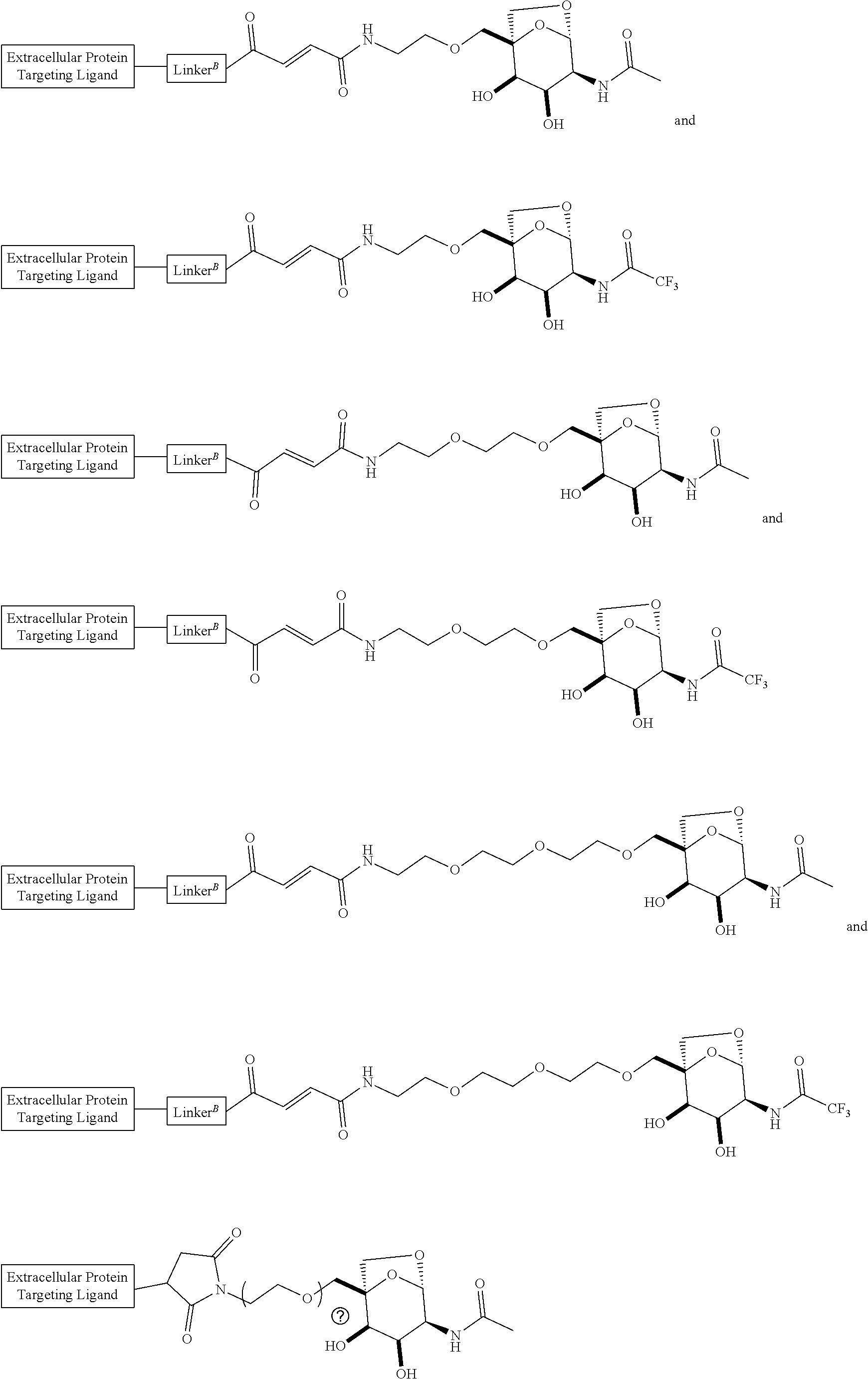

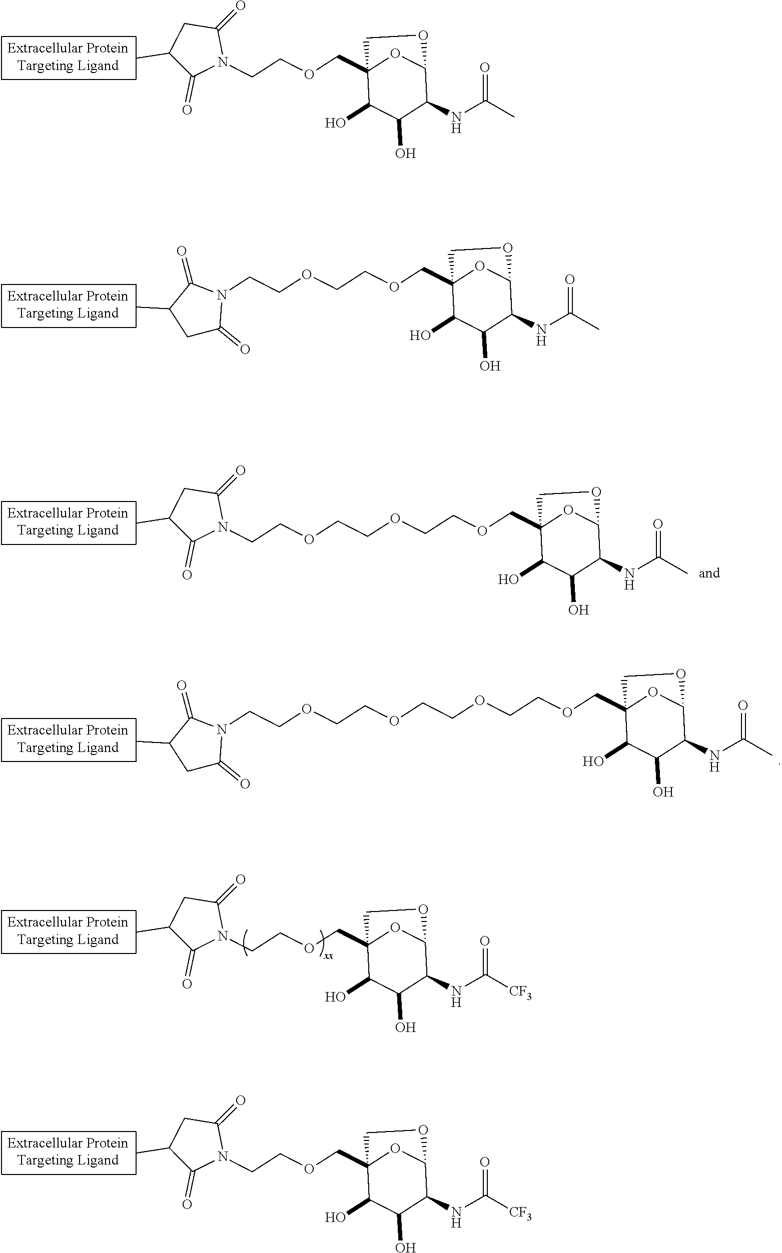

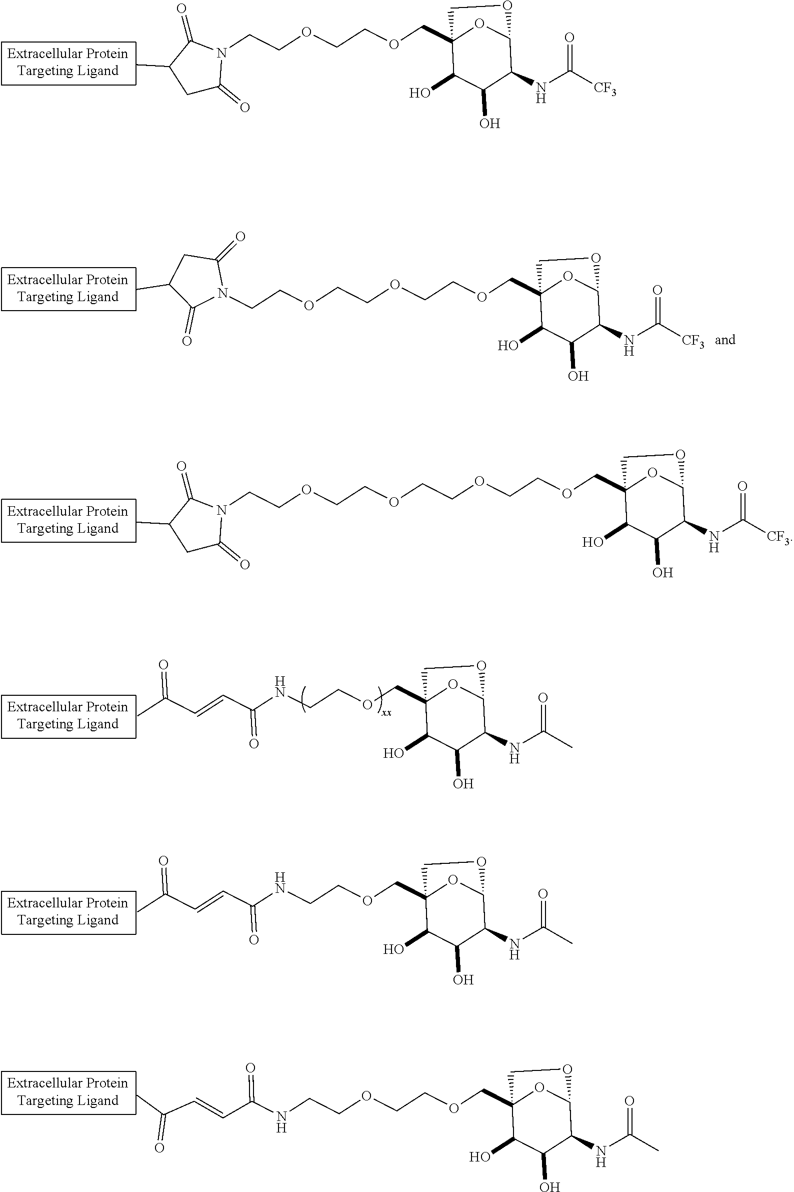

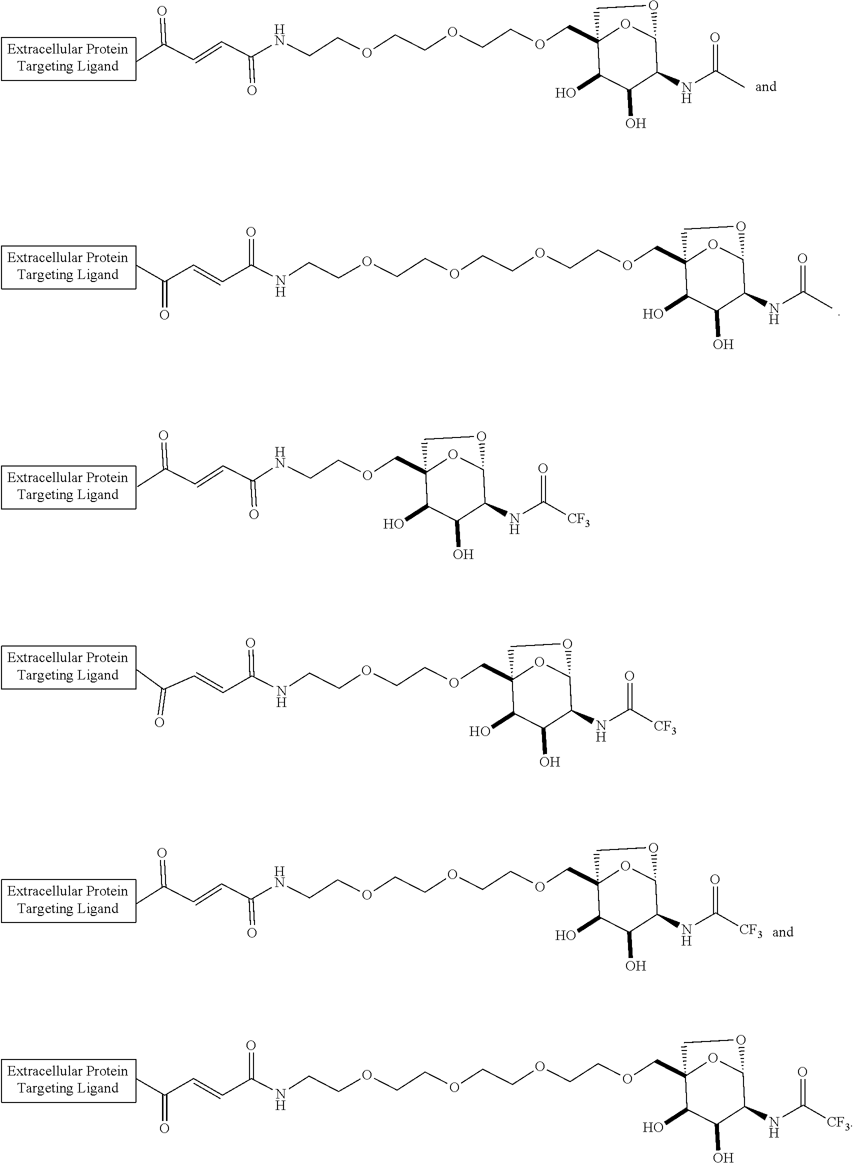

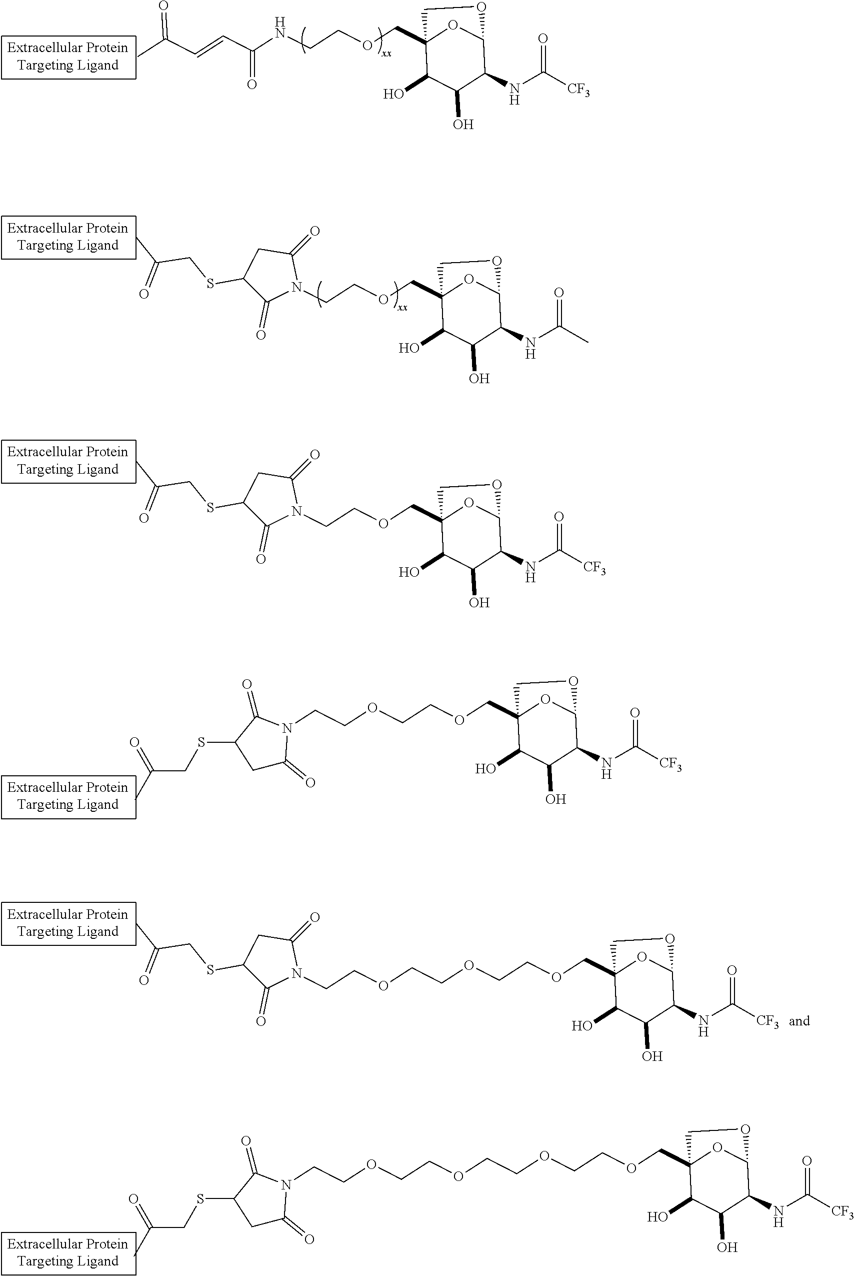

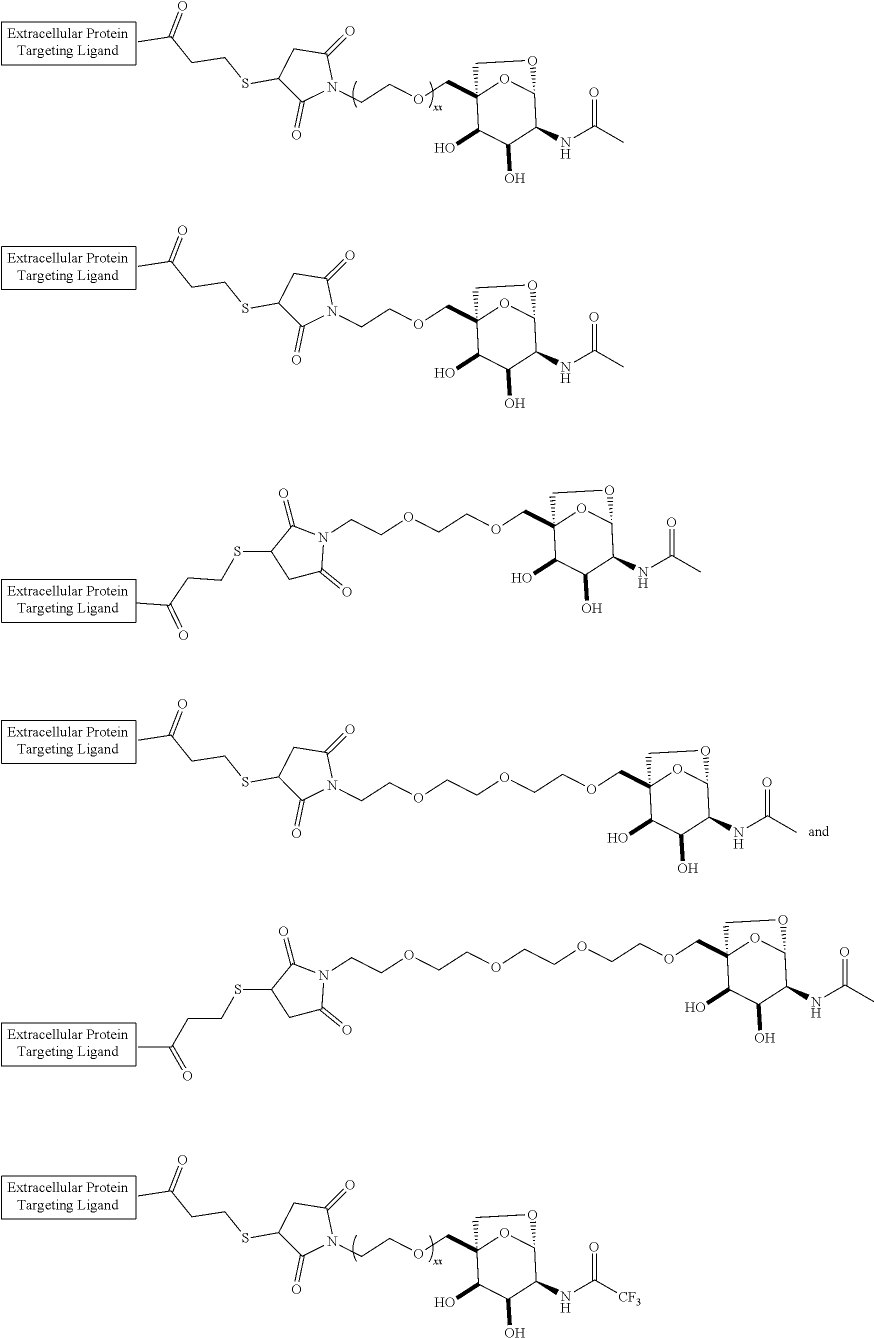

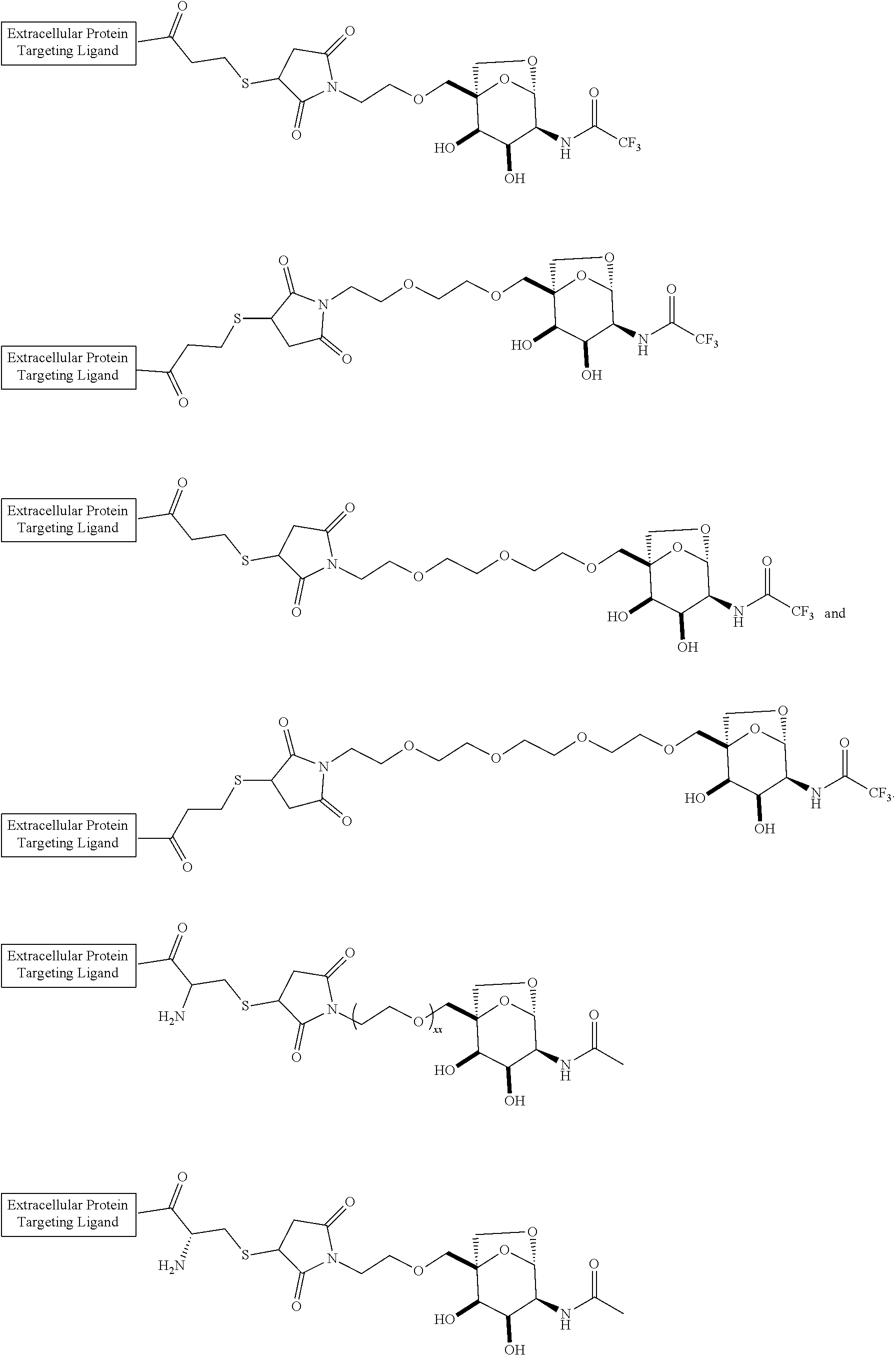

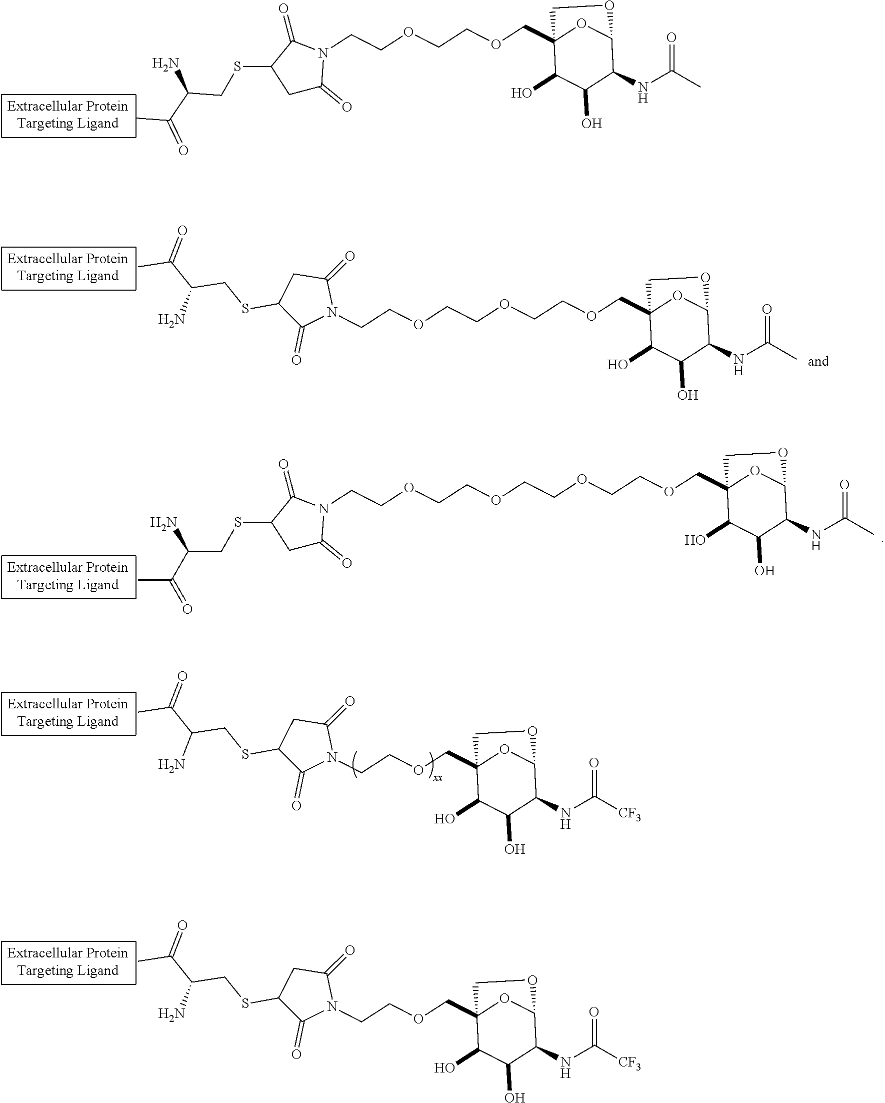

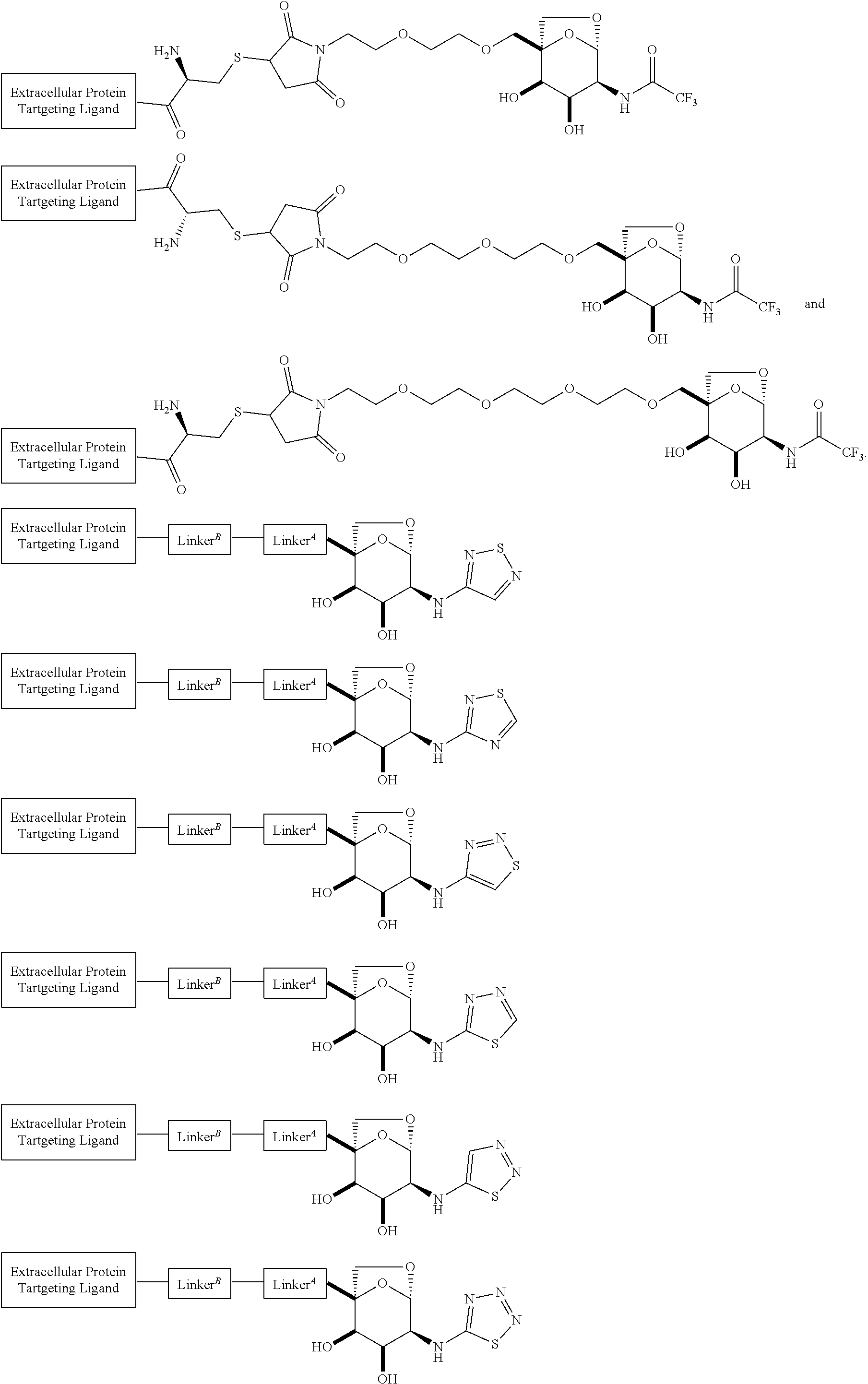

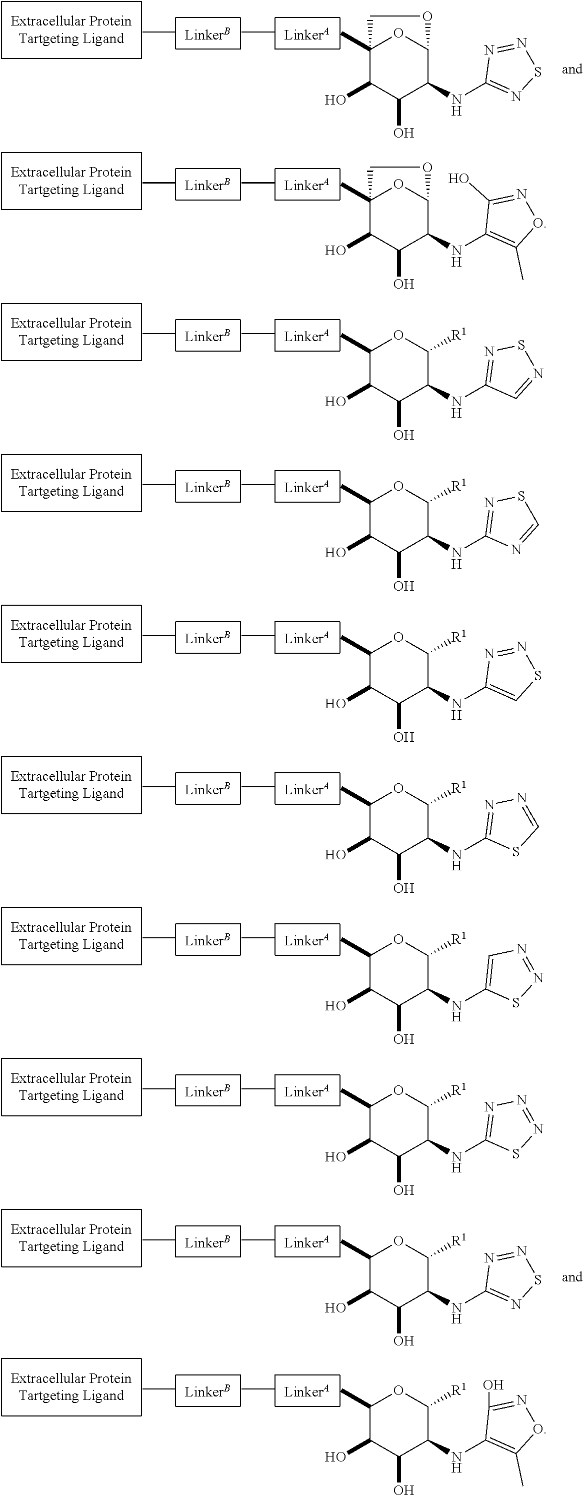

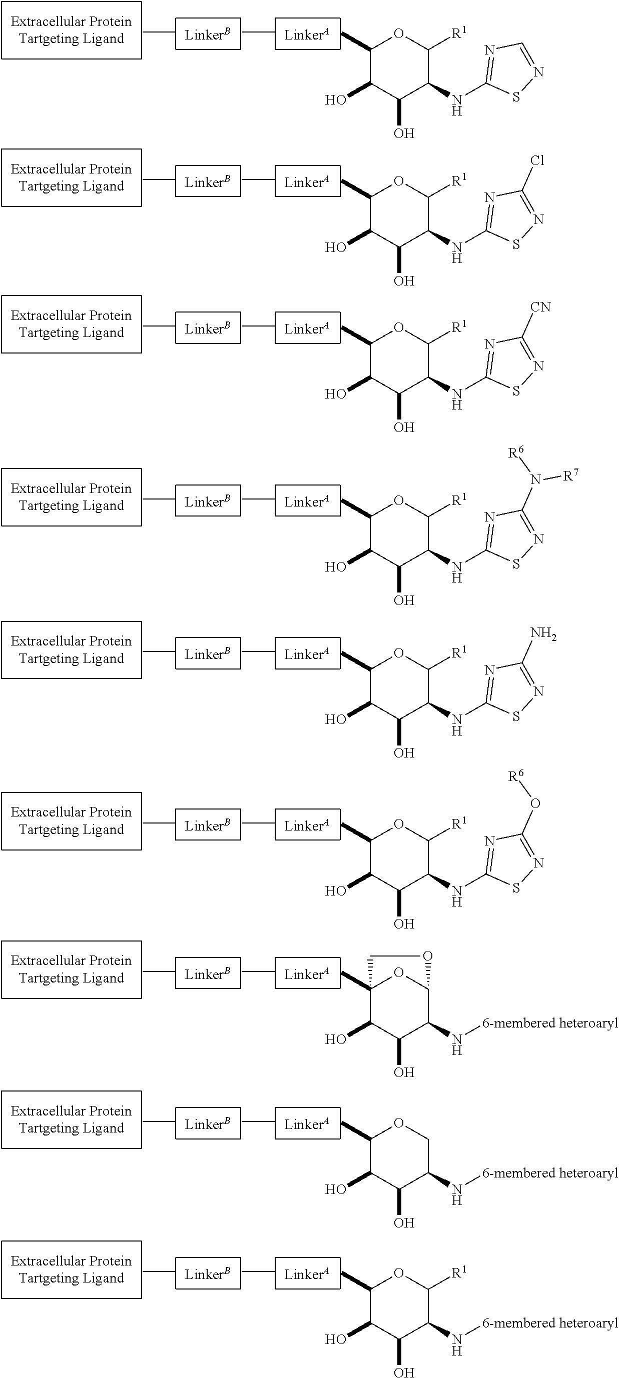

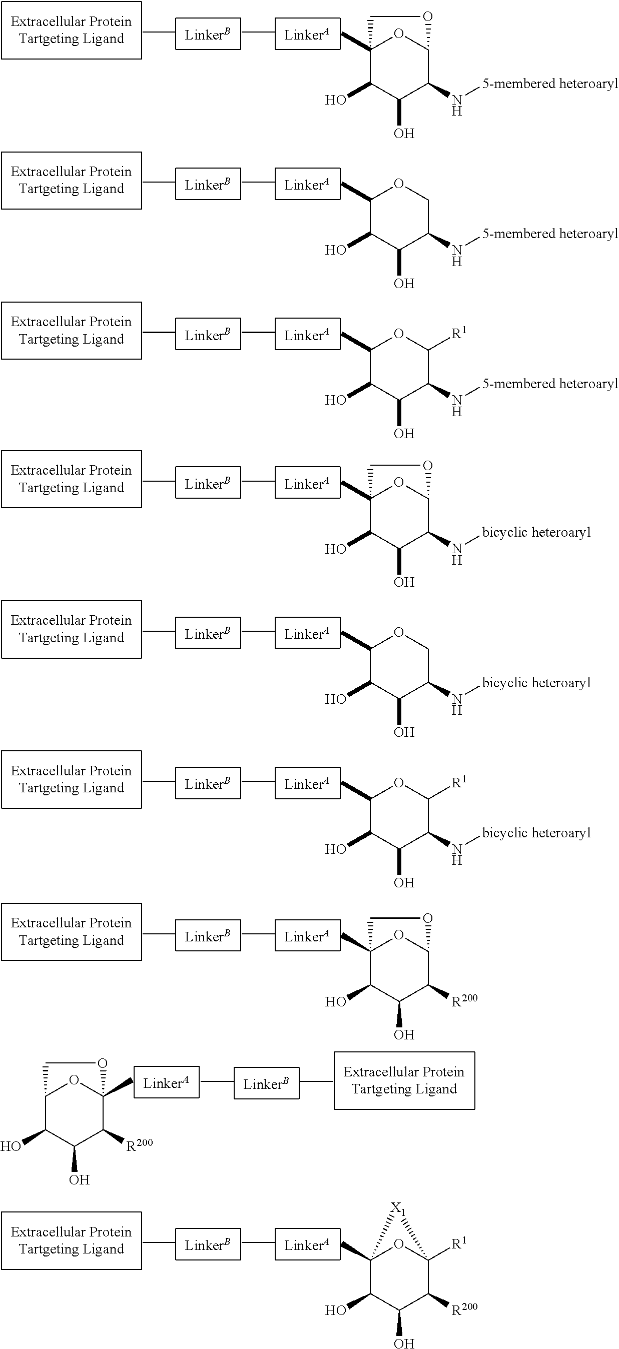

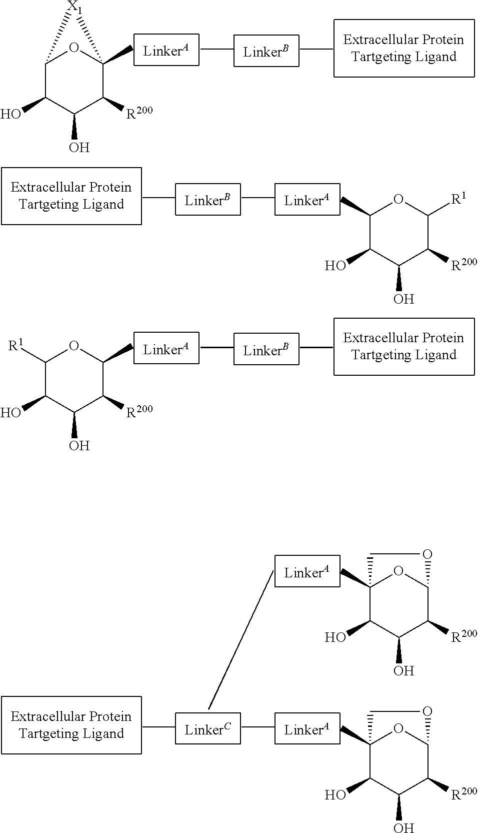

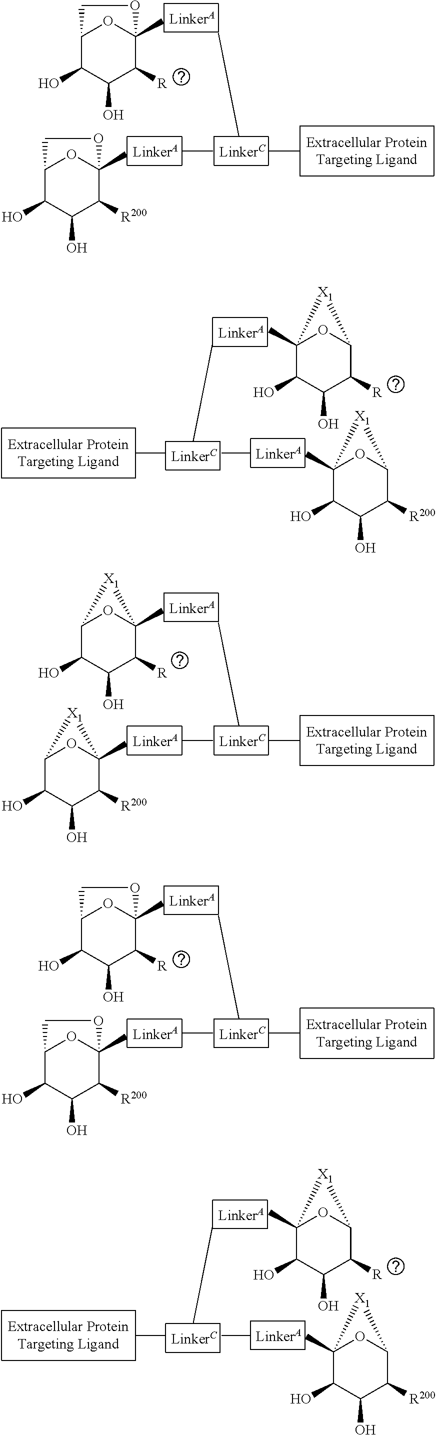

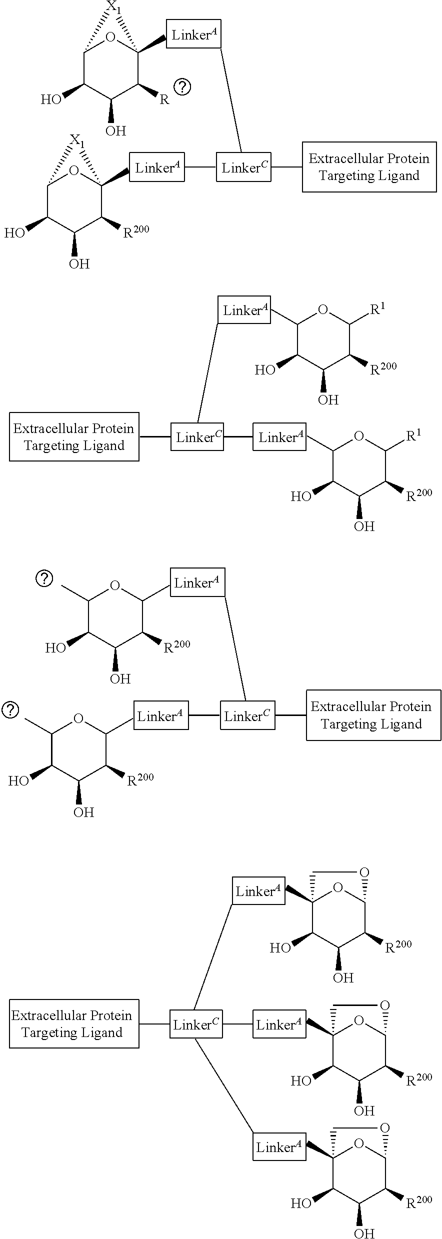

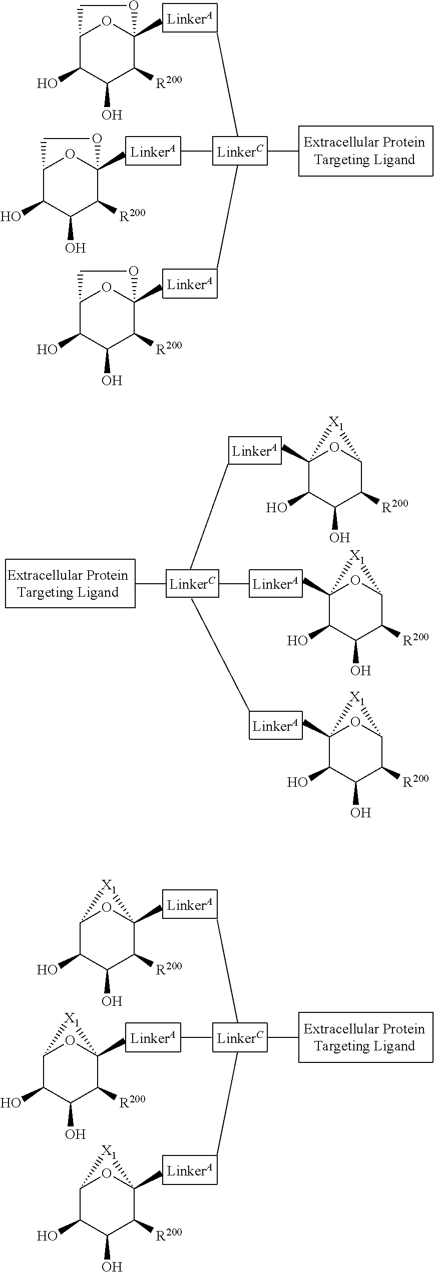

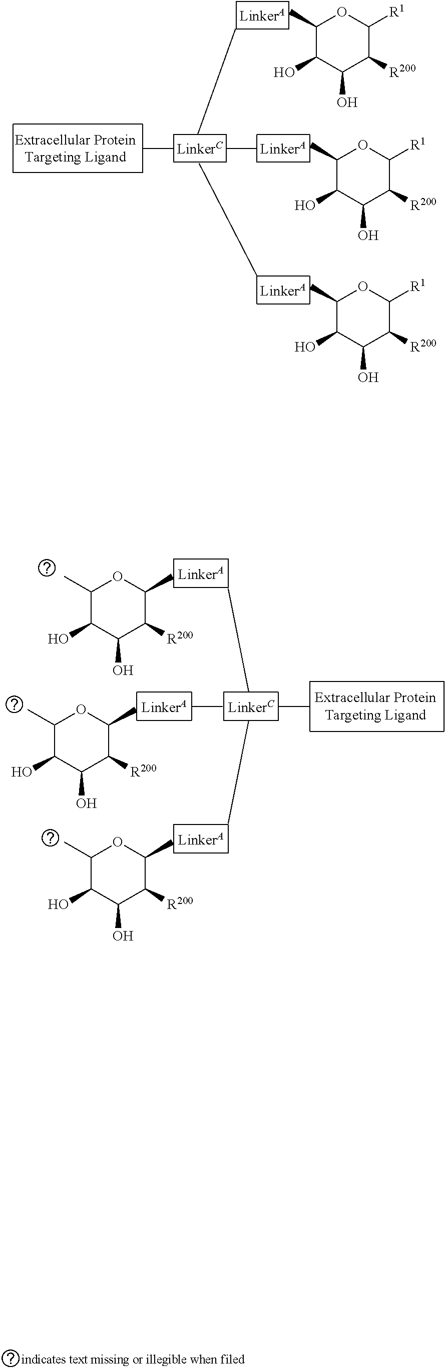

In certain embodiments, the compound of Formula II is an Extracellular Protein degrading compound in which the ASGPR ligand is a ligand as described herein

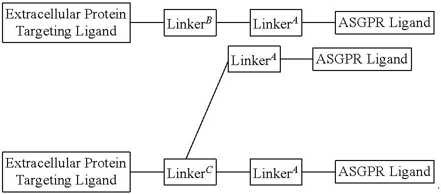

In certain embodiments, in the compound of Formula II, the ASGPR ligand is linked at either the C1 or C5 (R¹ or R⁵) position to form a degrading compound. In certain embodiments, in the compound of Formula II, the ASGPR ligand is linked at C6. In various embodiments, when the ASGPR ligand is

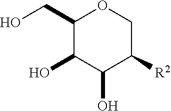

then non-limiting examples of ASGPR binding compounds of Formula II include:

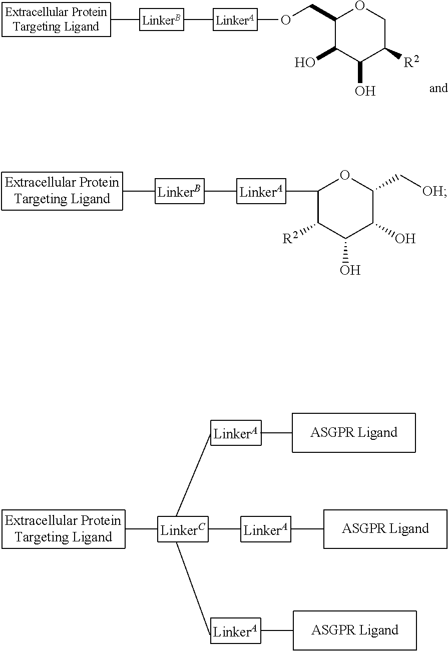

or the bi- or tri-substituted versions thereof or pharmaceutically acceptable salts thereof, where the bi- or tri-substitution refers to the number additional galactose derivatives attached to a linker moiety. In certain embodiments the compound of Formula II is selected from:

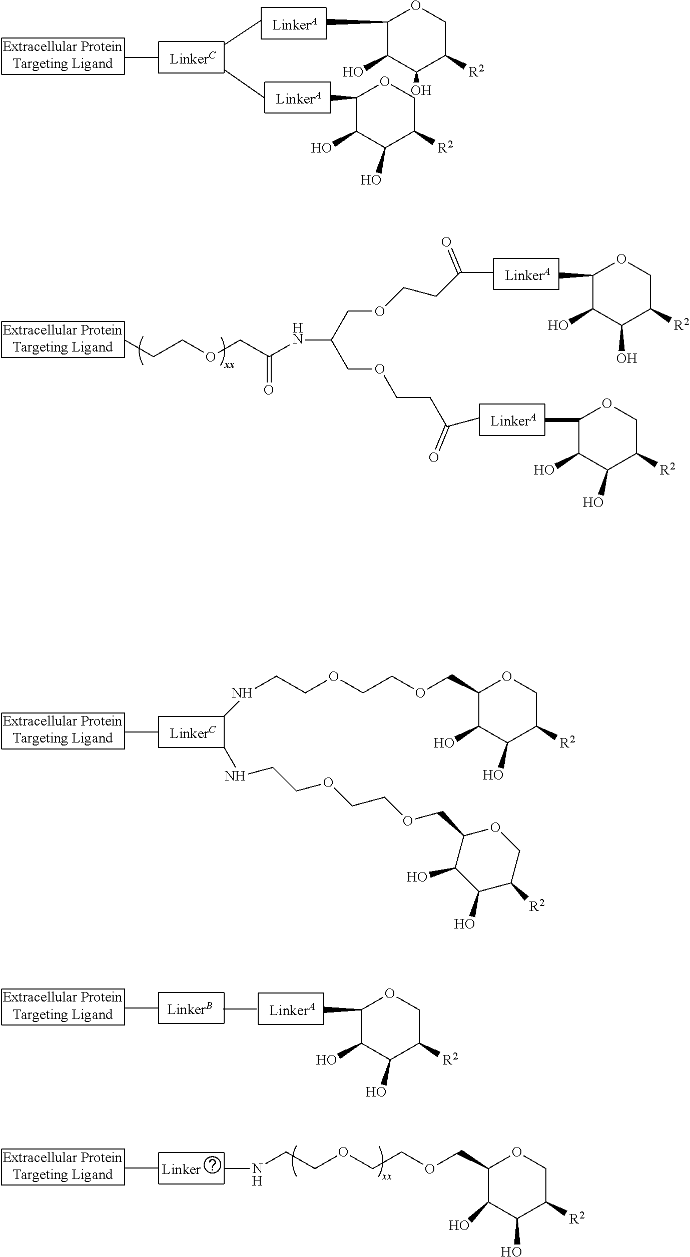

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

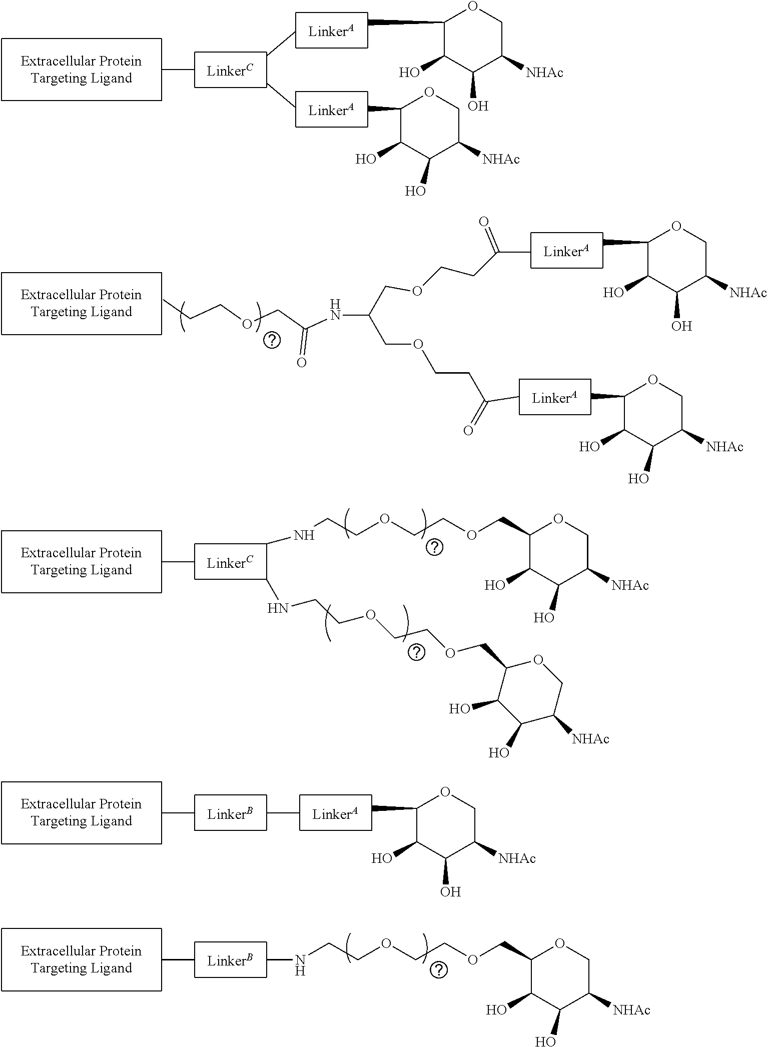

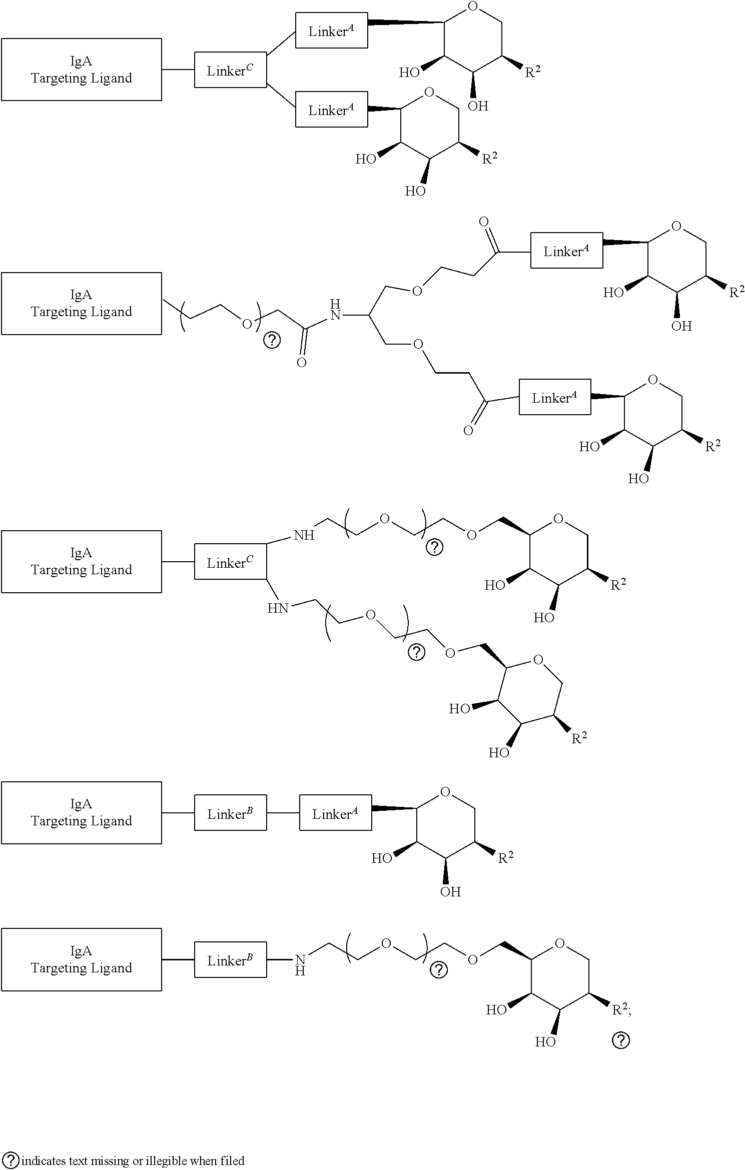

wherein in certain embodiments R¹ is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

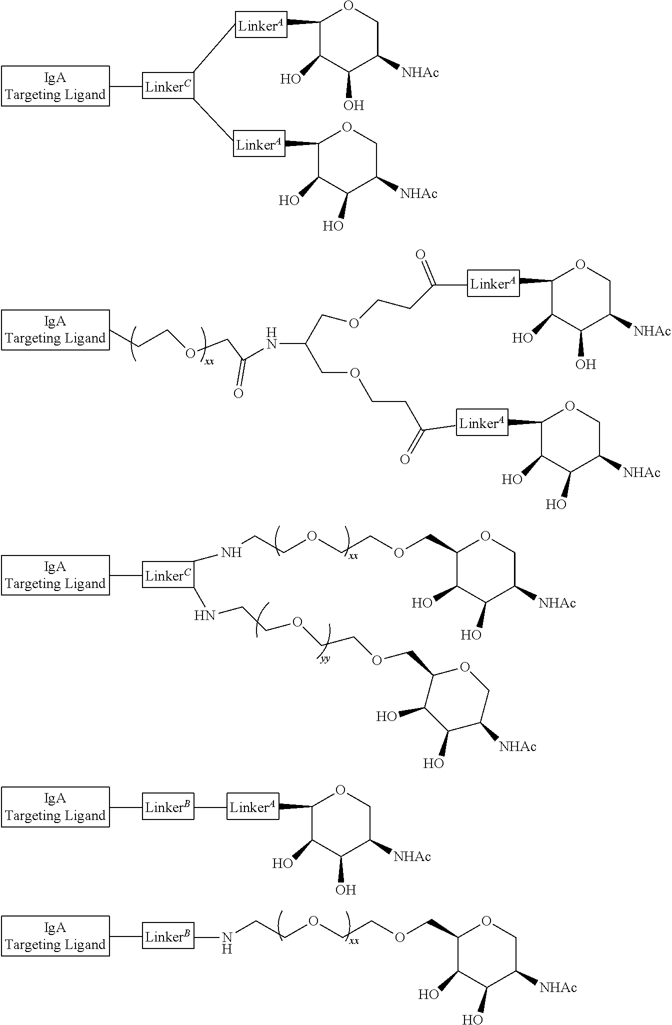

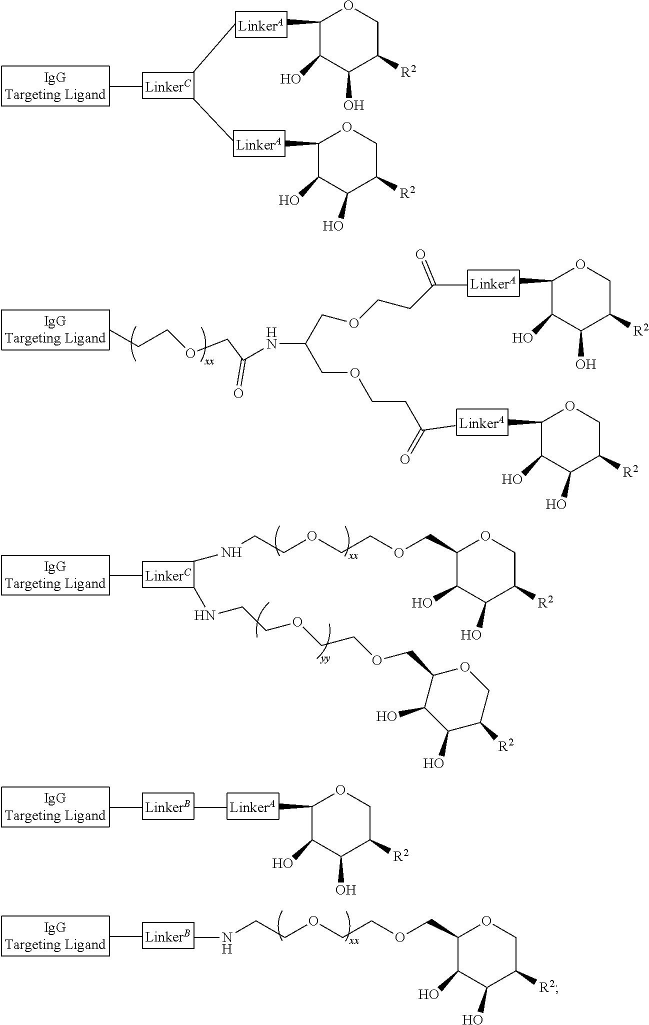

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

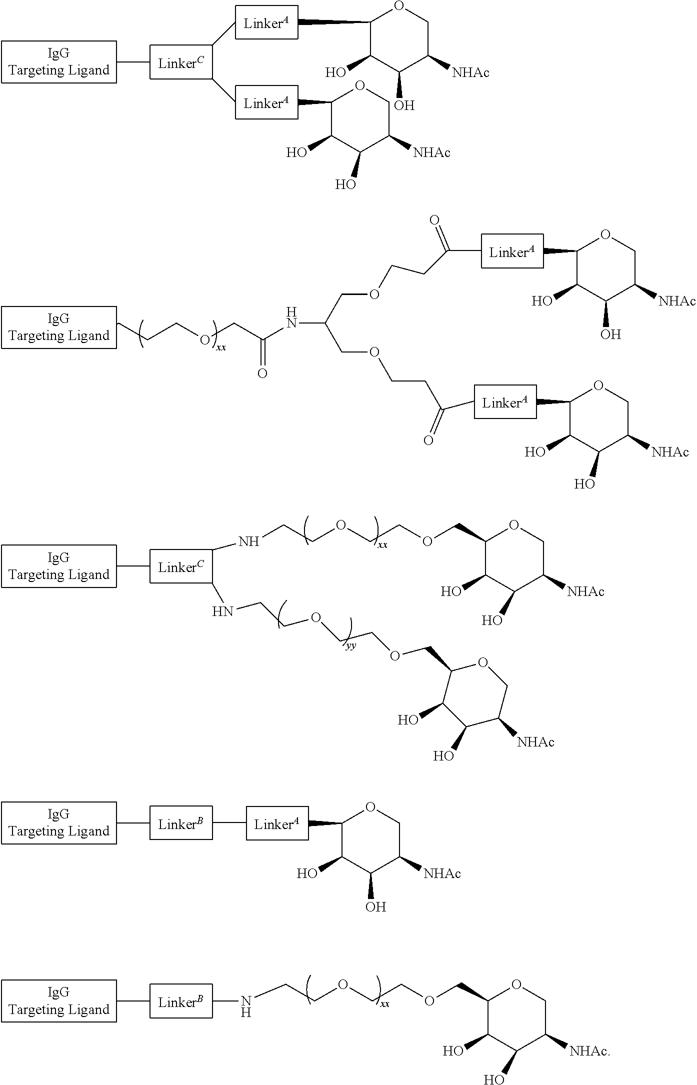

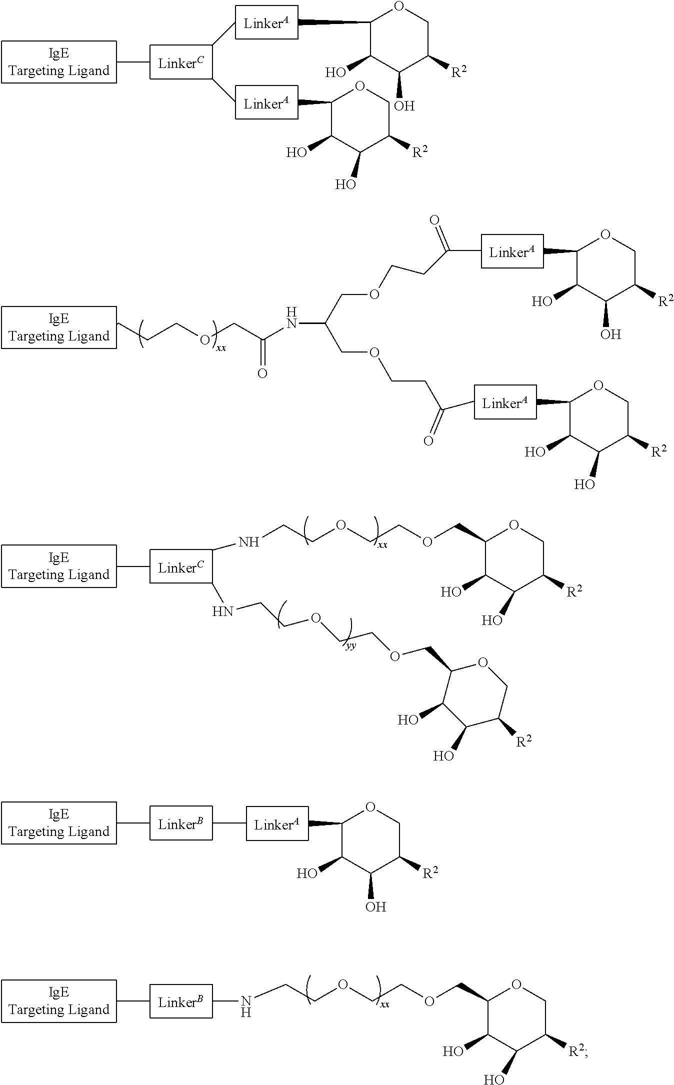

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

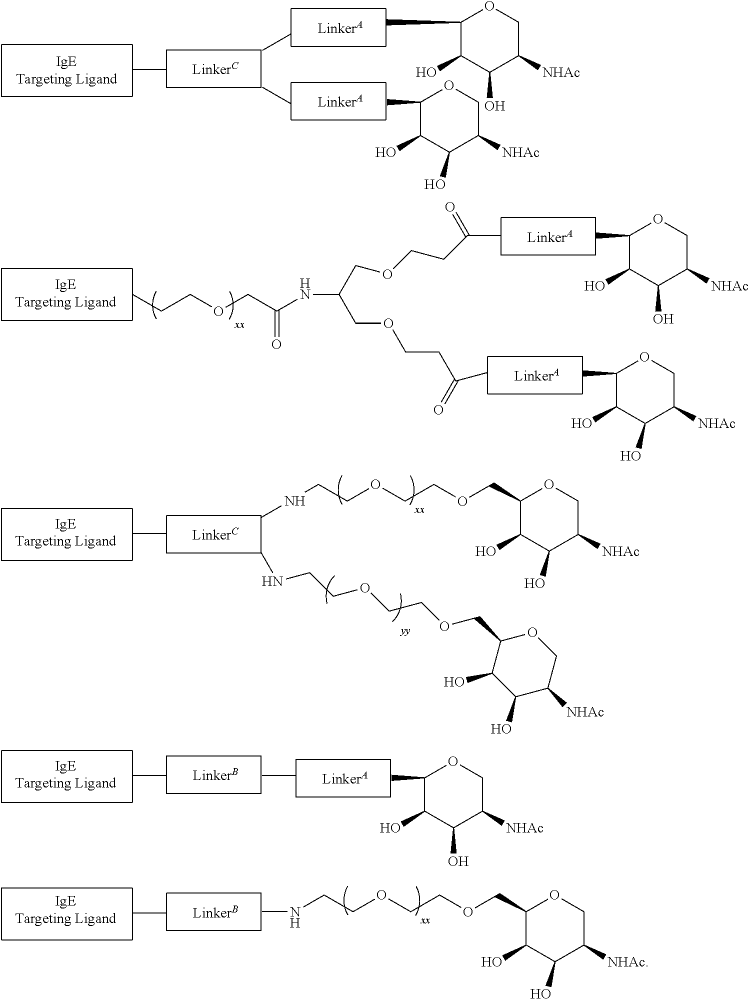

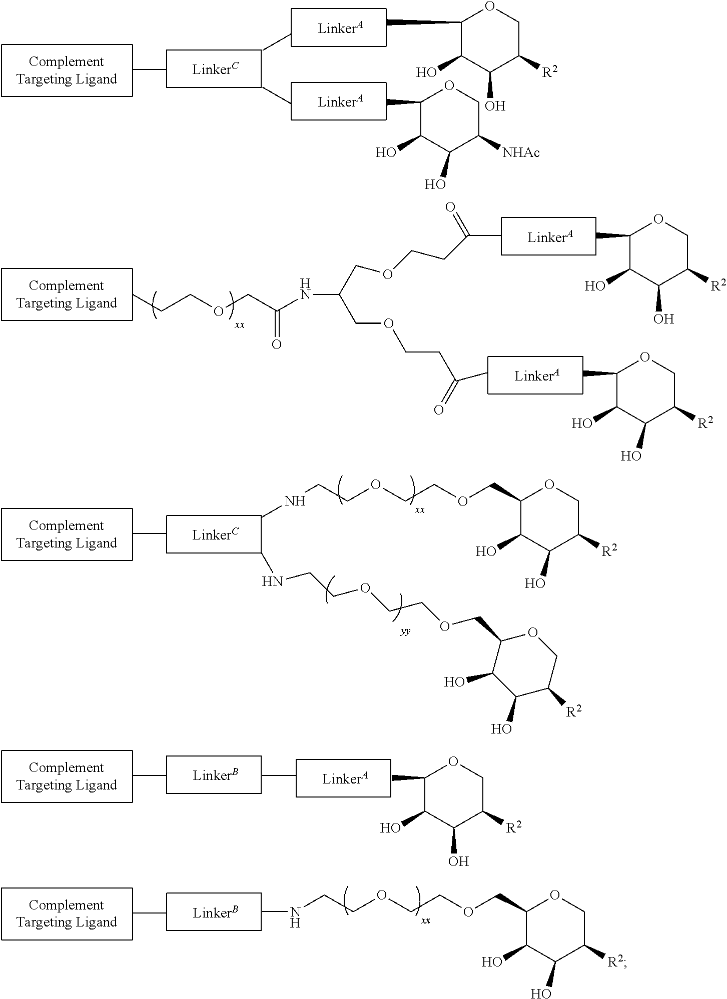

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

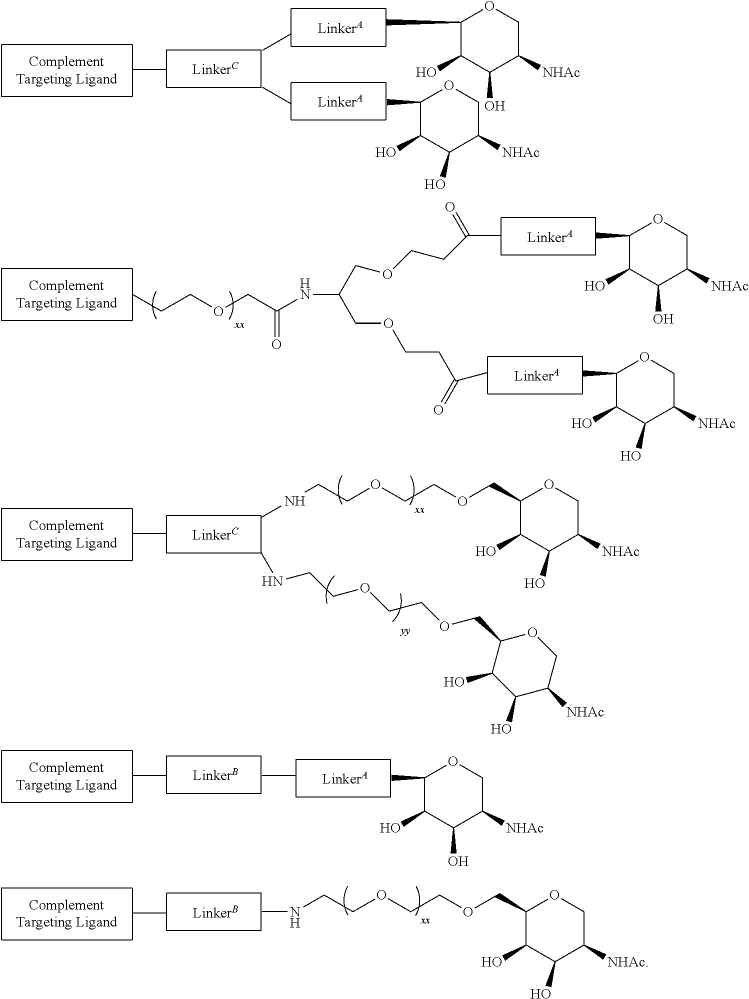

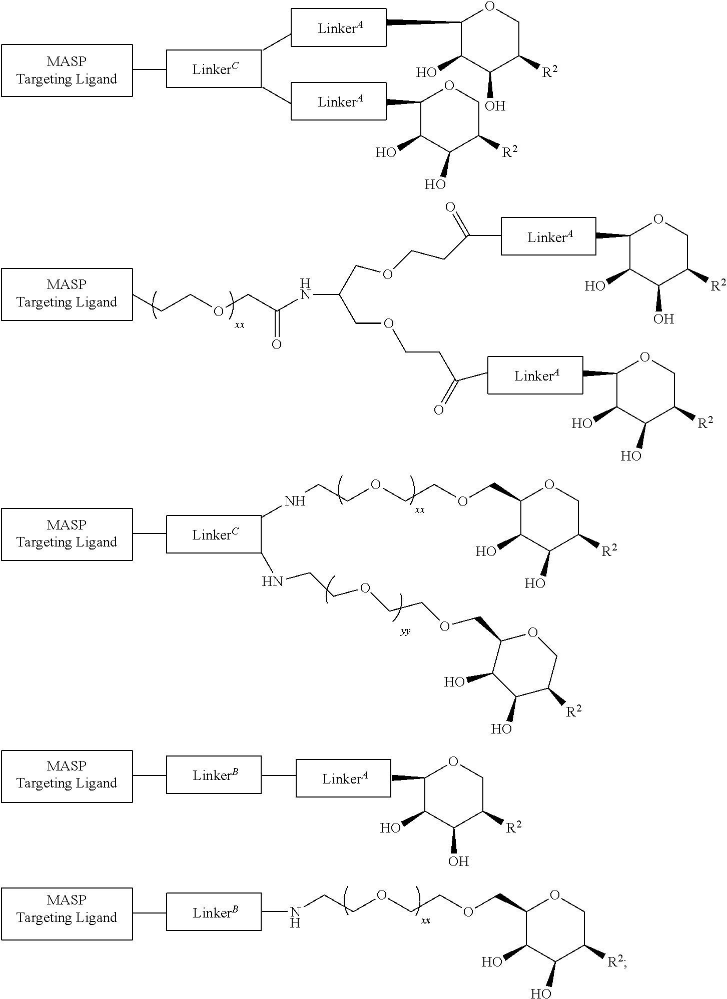

wherein in certain embodiments R² is selected from —NR⁶COR³, —NR -(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

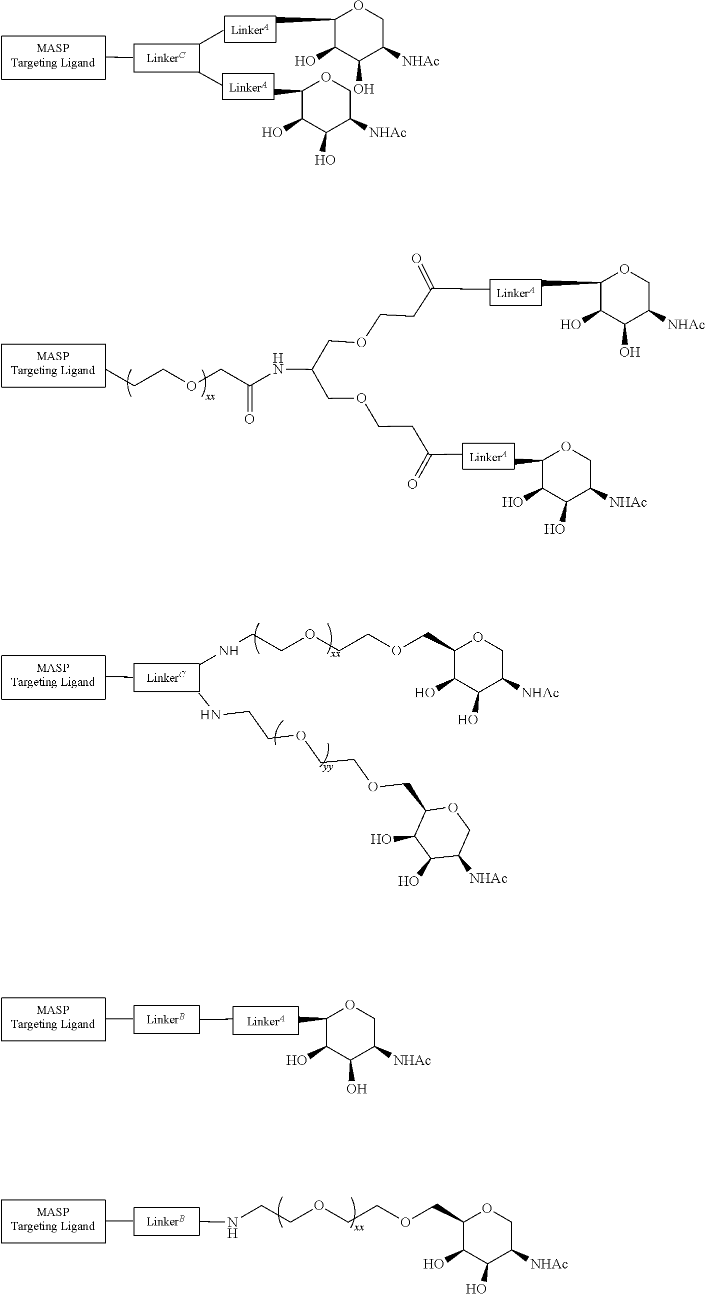

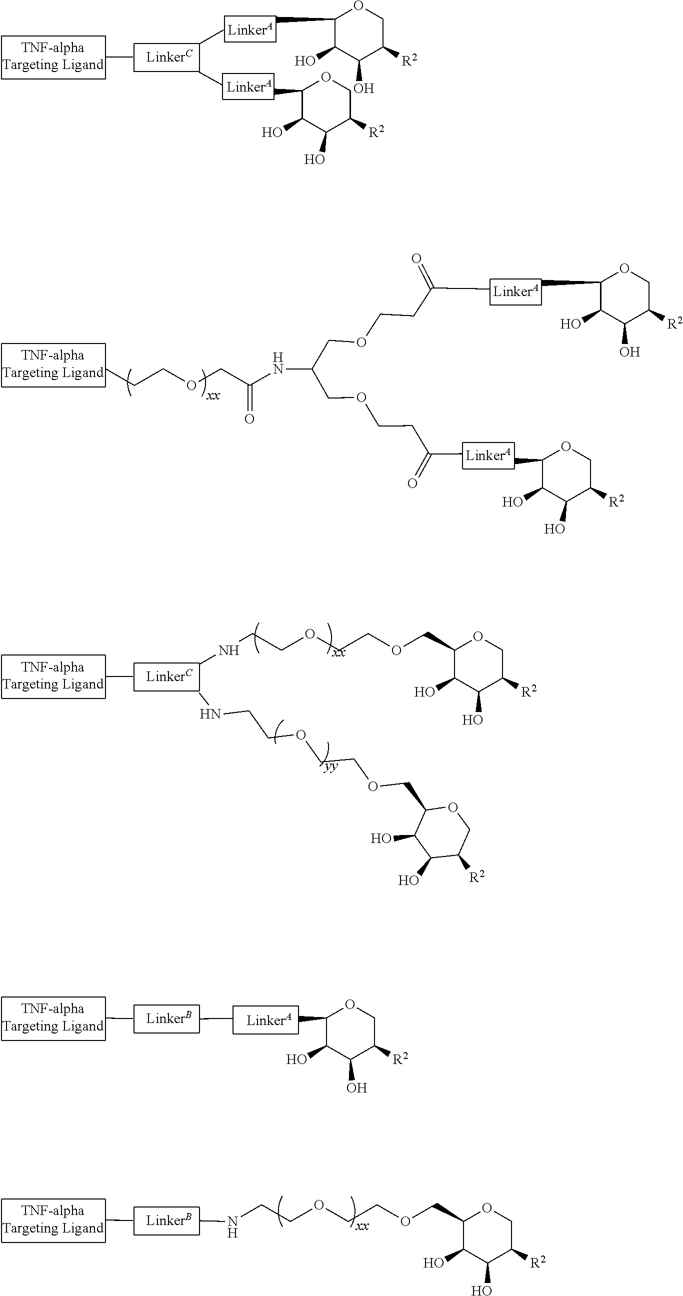

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

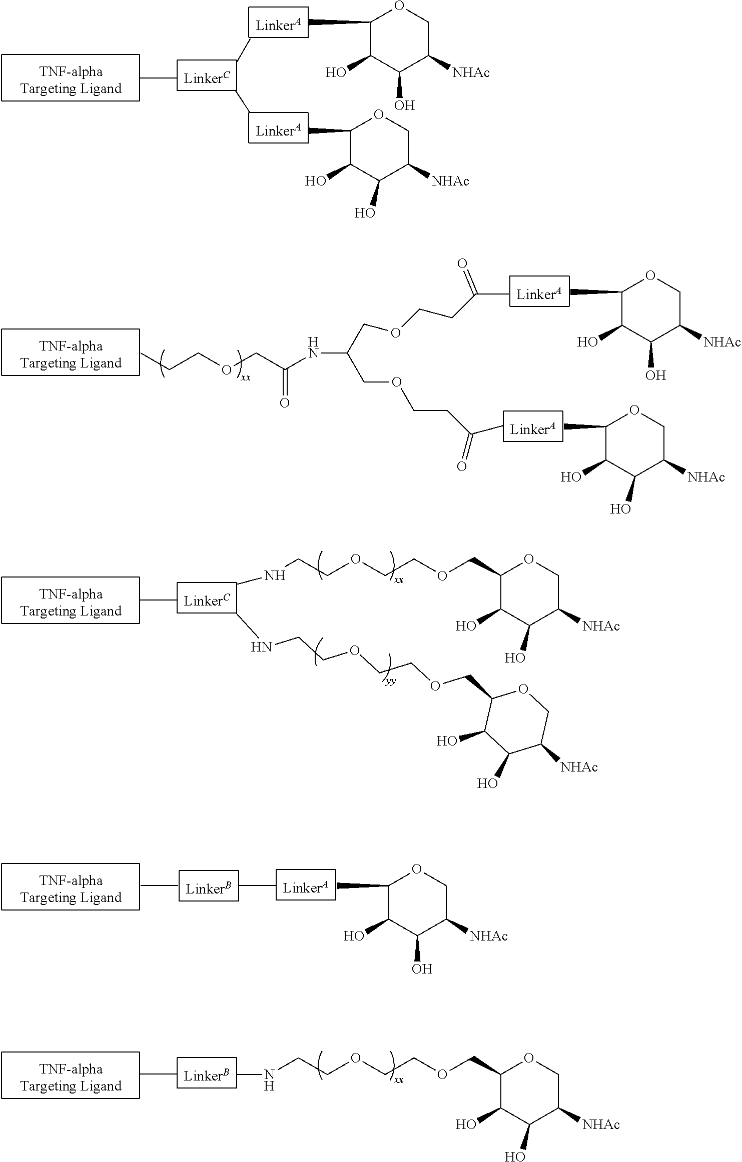

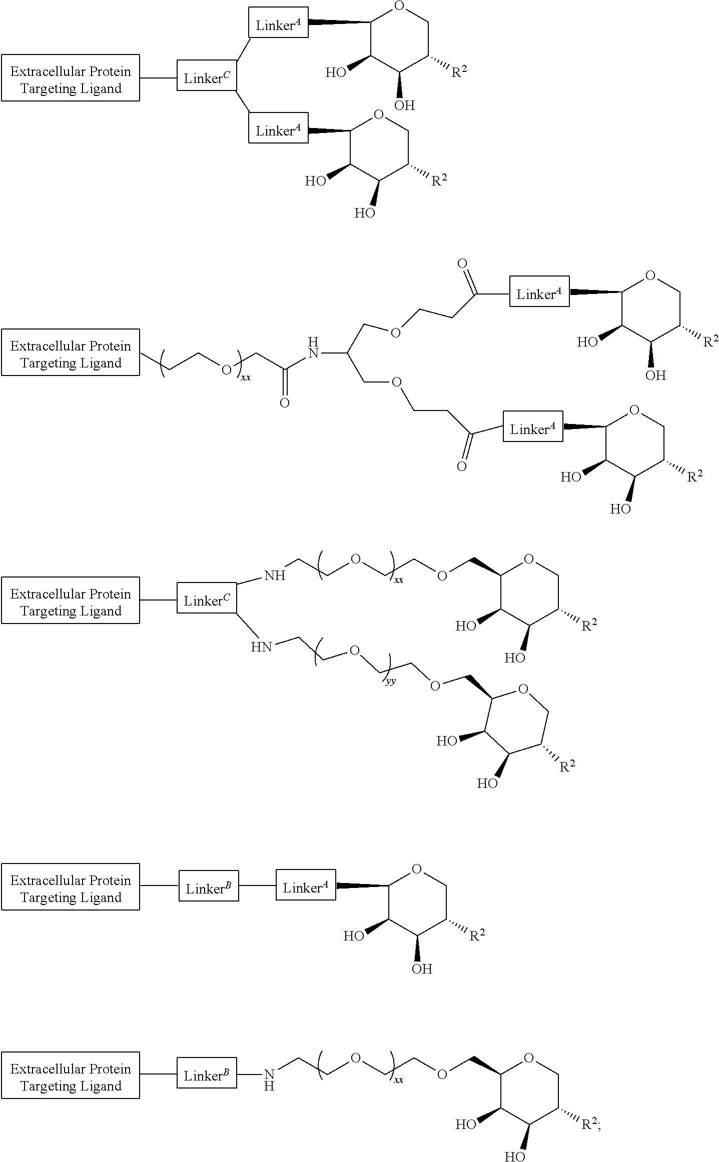

wherein in certain embodiments R² is selected from —NR⁶COR¹⁰, —NR⁶-5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

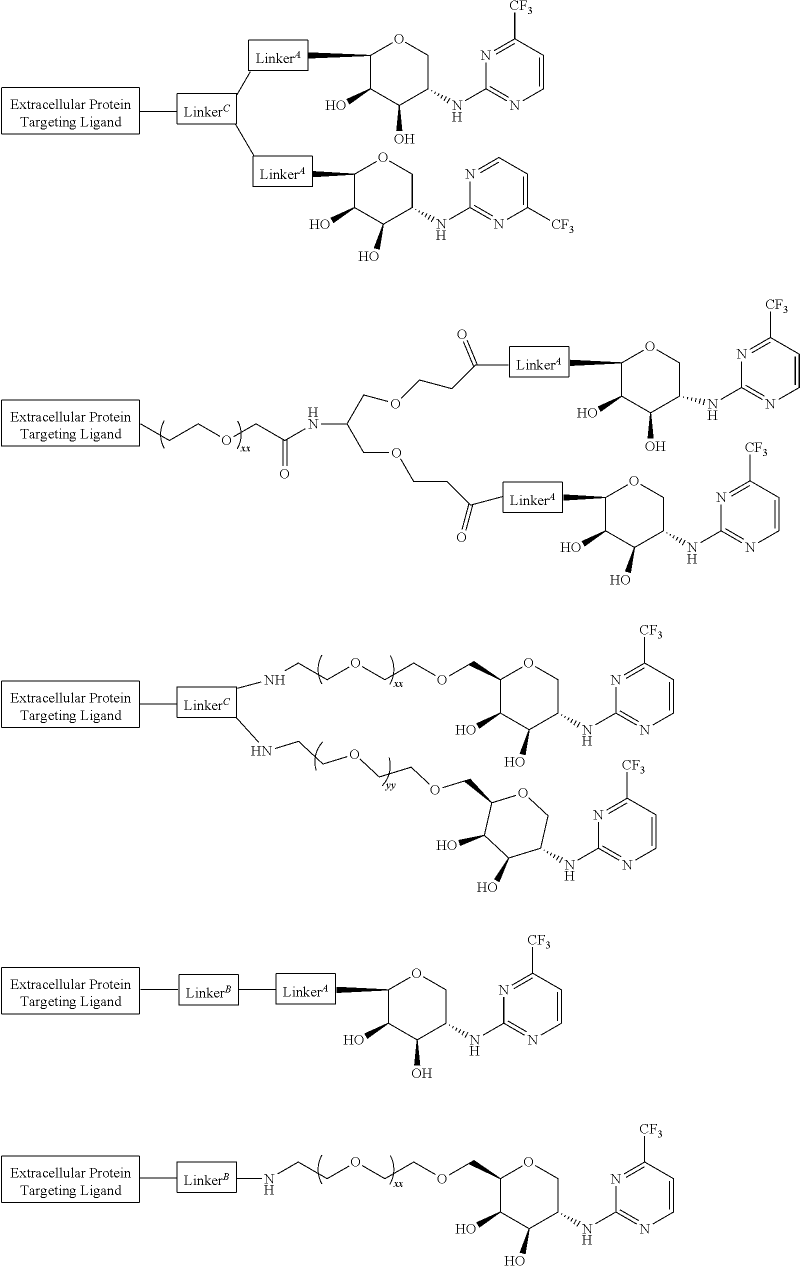

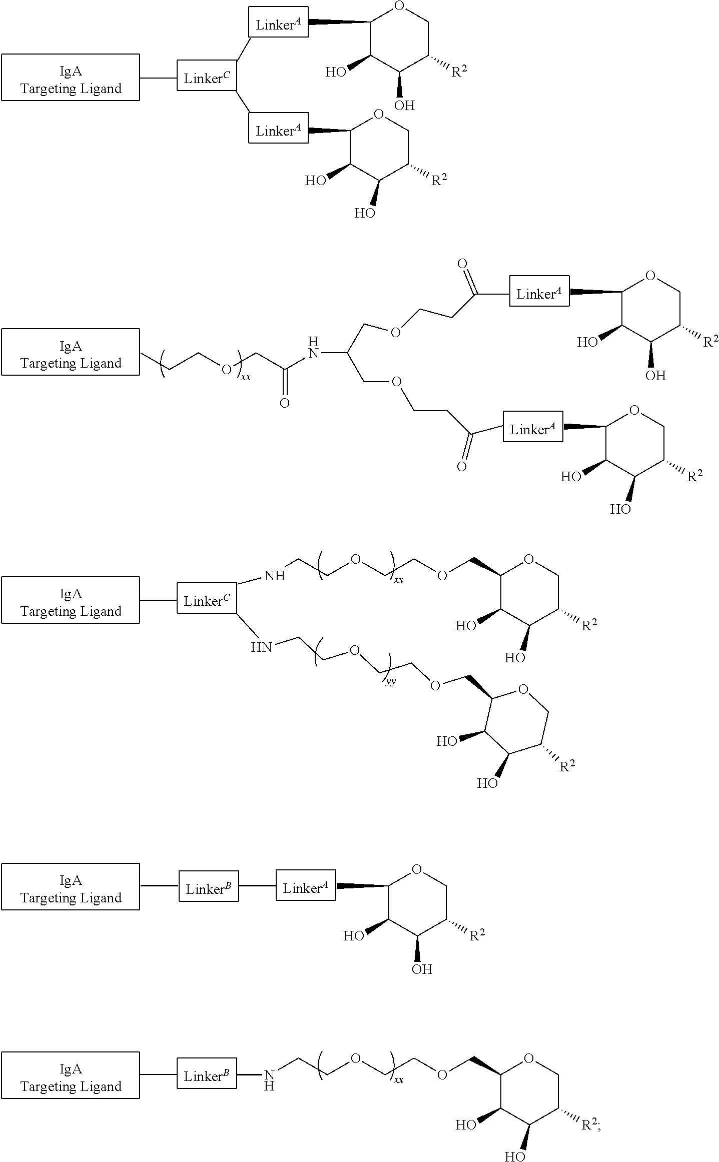

wherein in certain embodiments R² is selected from —NR^{b}COR¹⁰, —NR⁶-(5-membered heteroaryl), and —NR⁶-(6-membered heteroaryl), each of which R^{Z} groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

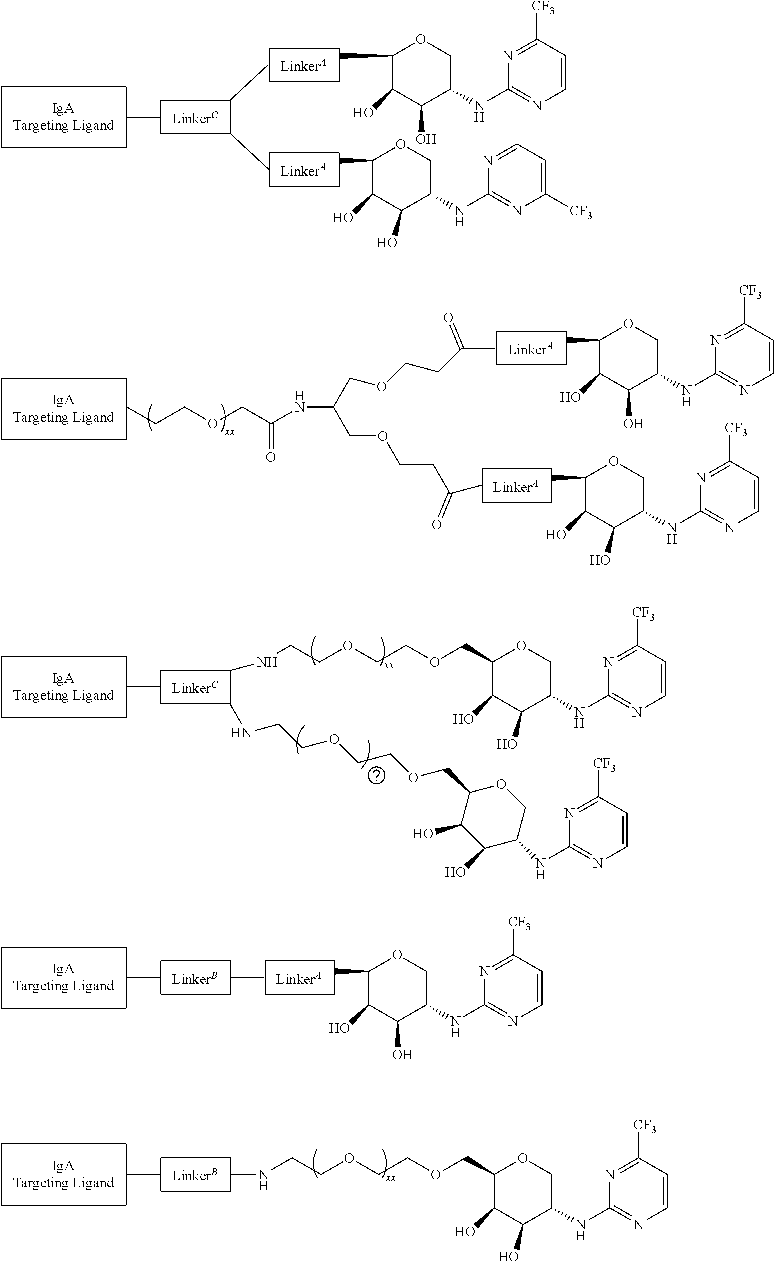

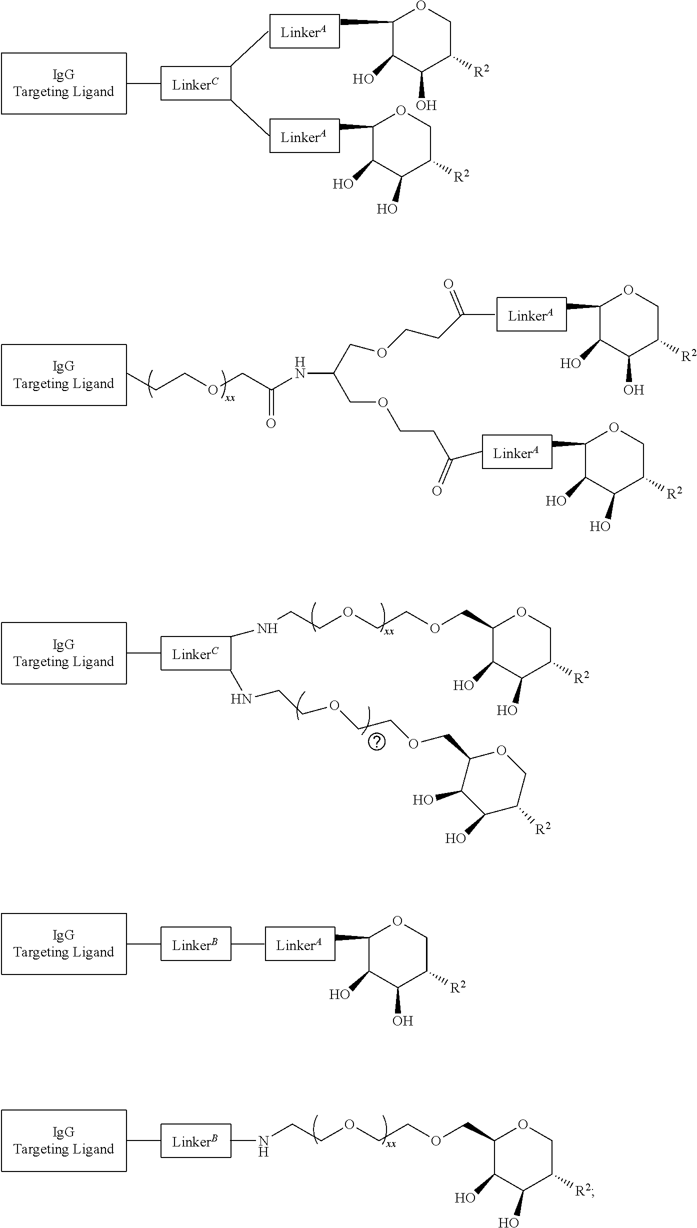

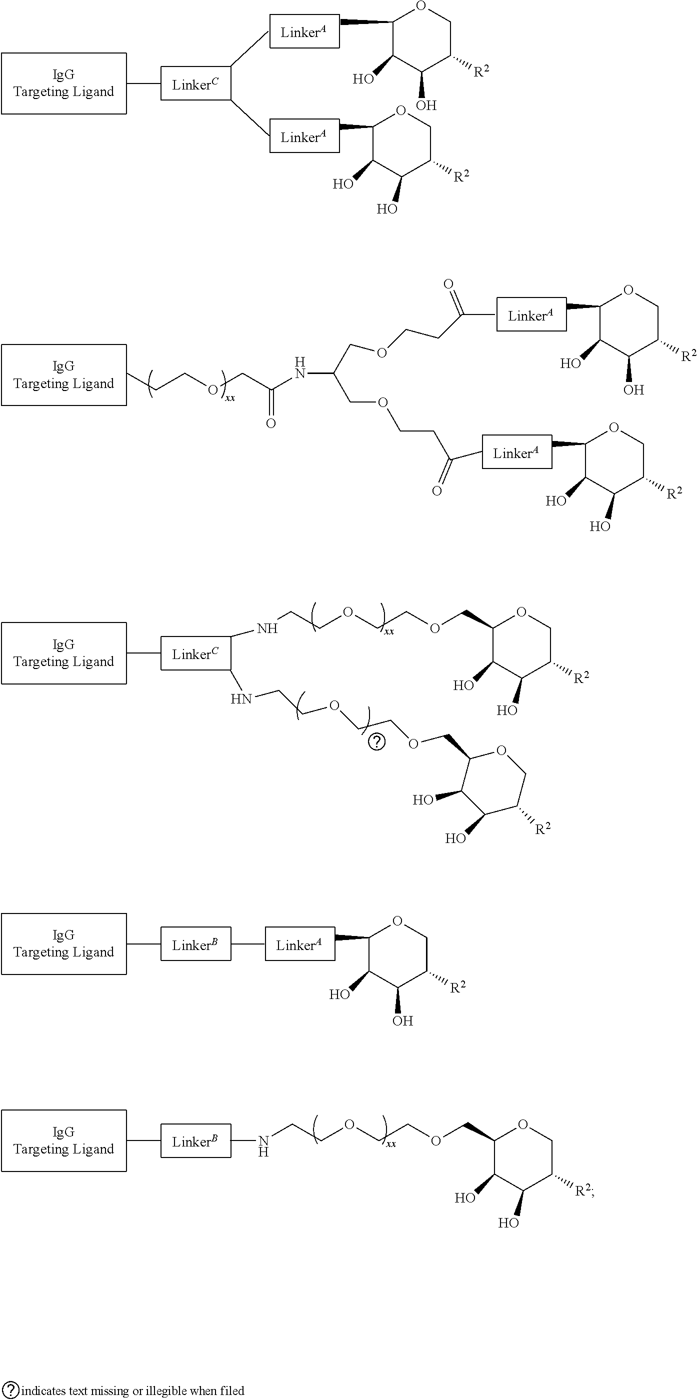

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

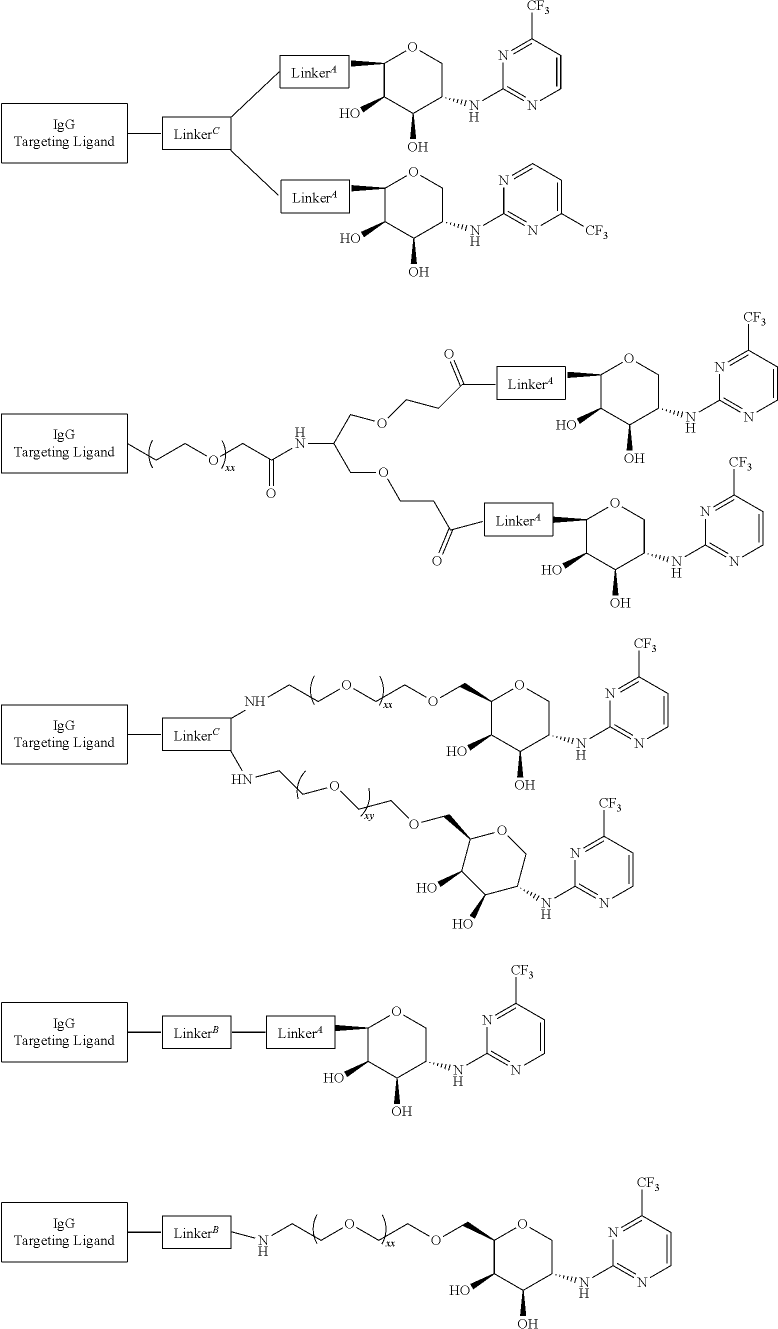

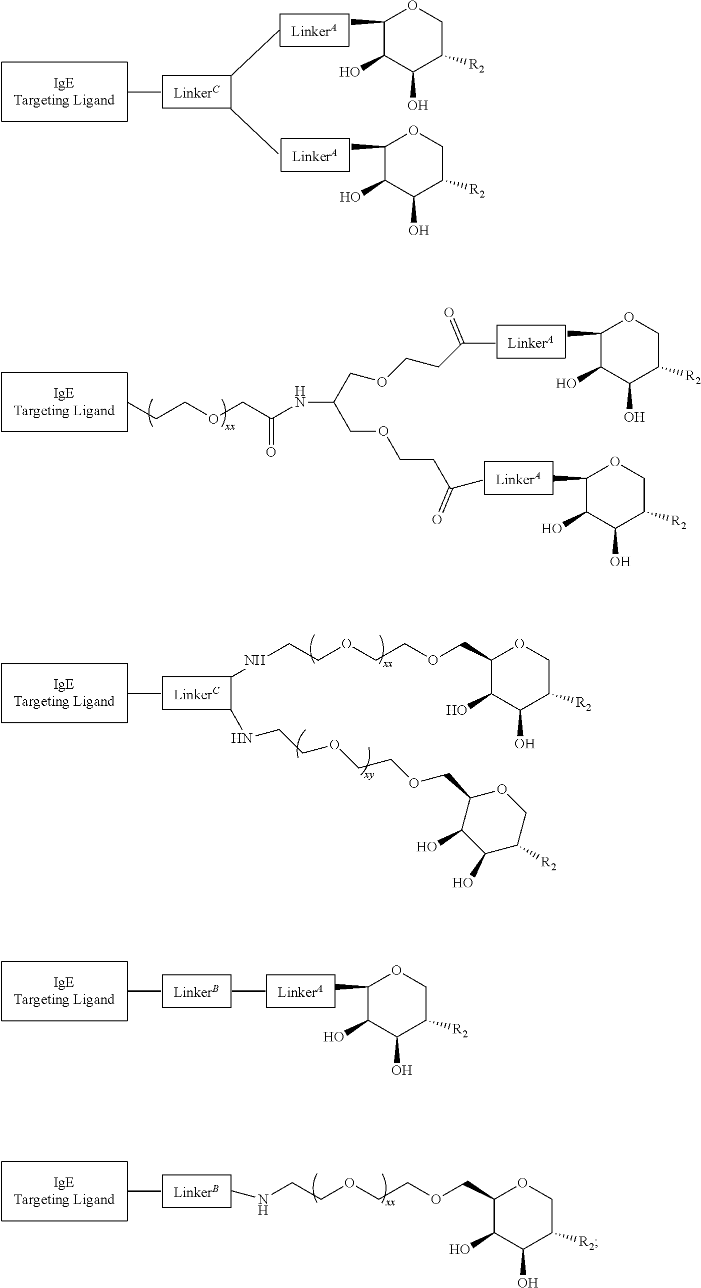

wherein in certain embodiments R² is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

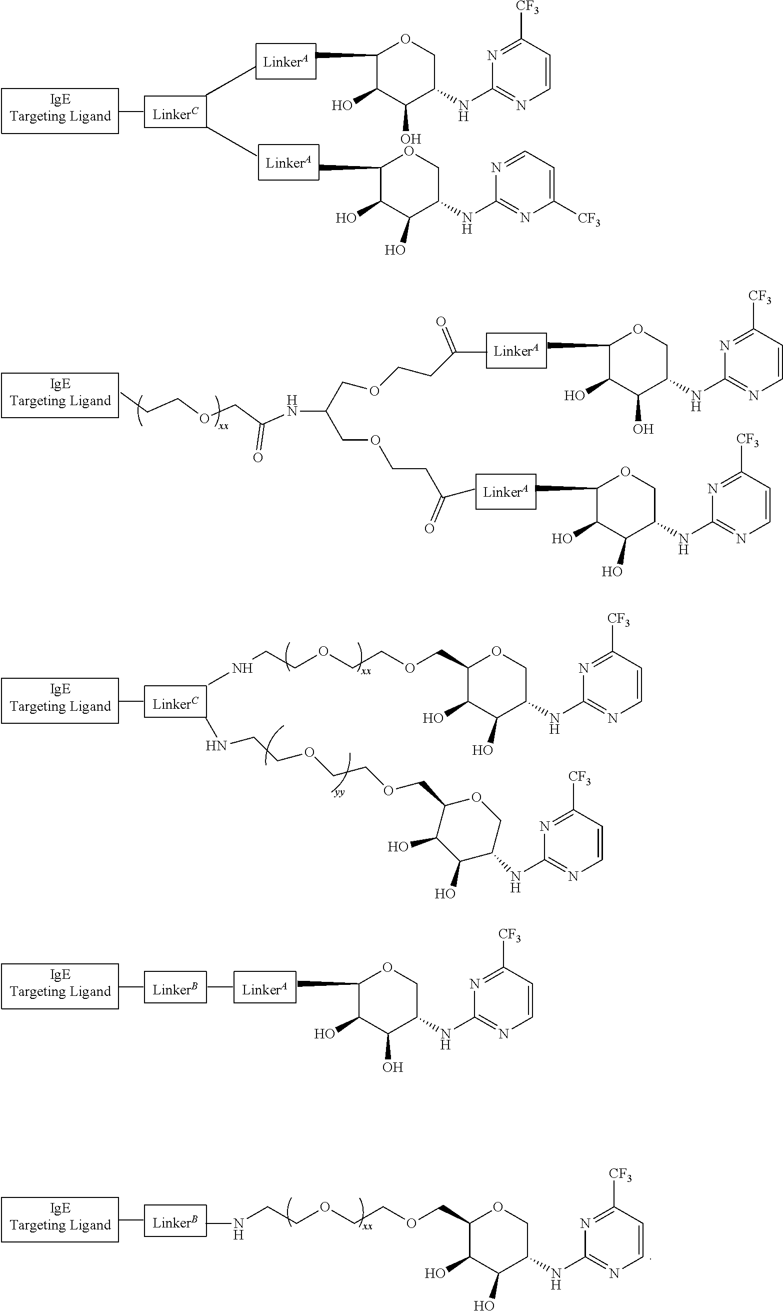

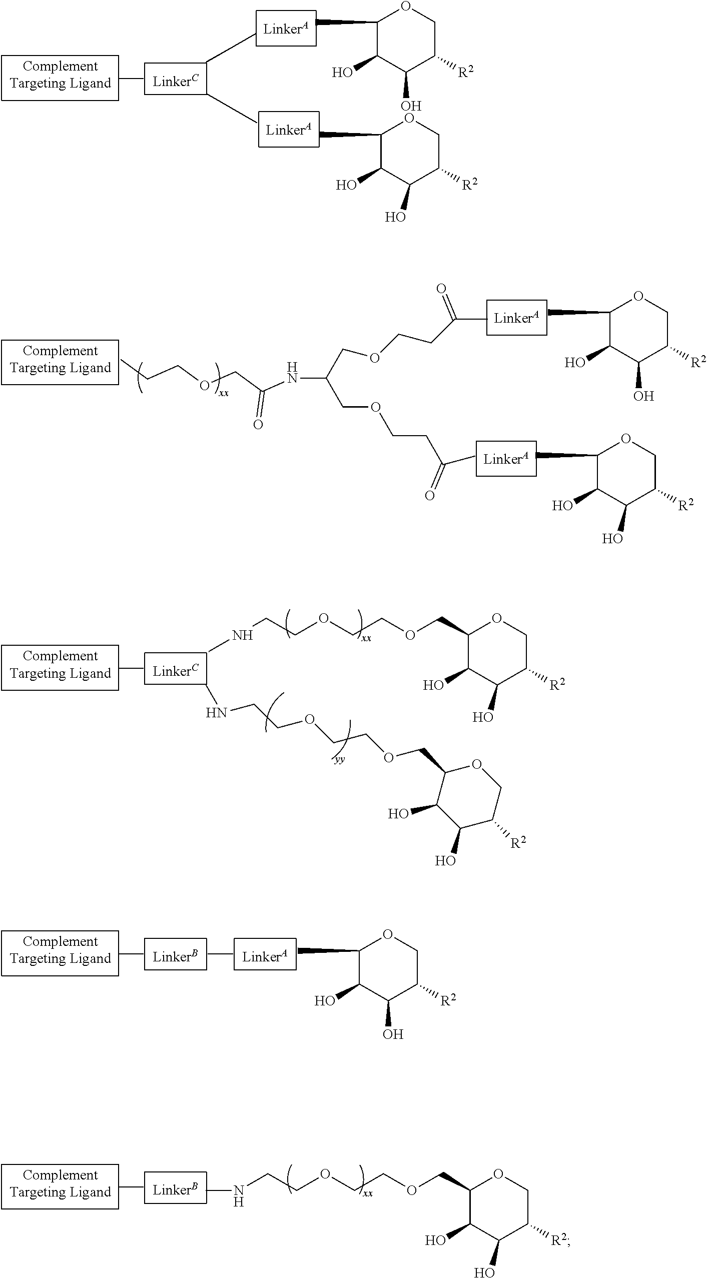

wherein in certain embodiments R¹ is selected from —NR⁶COR¹⁰, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

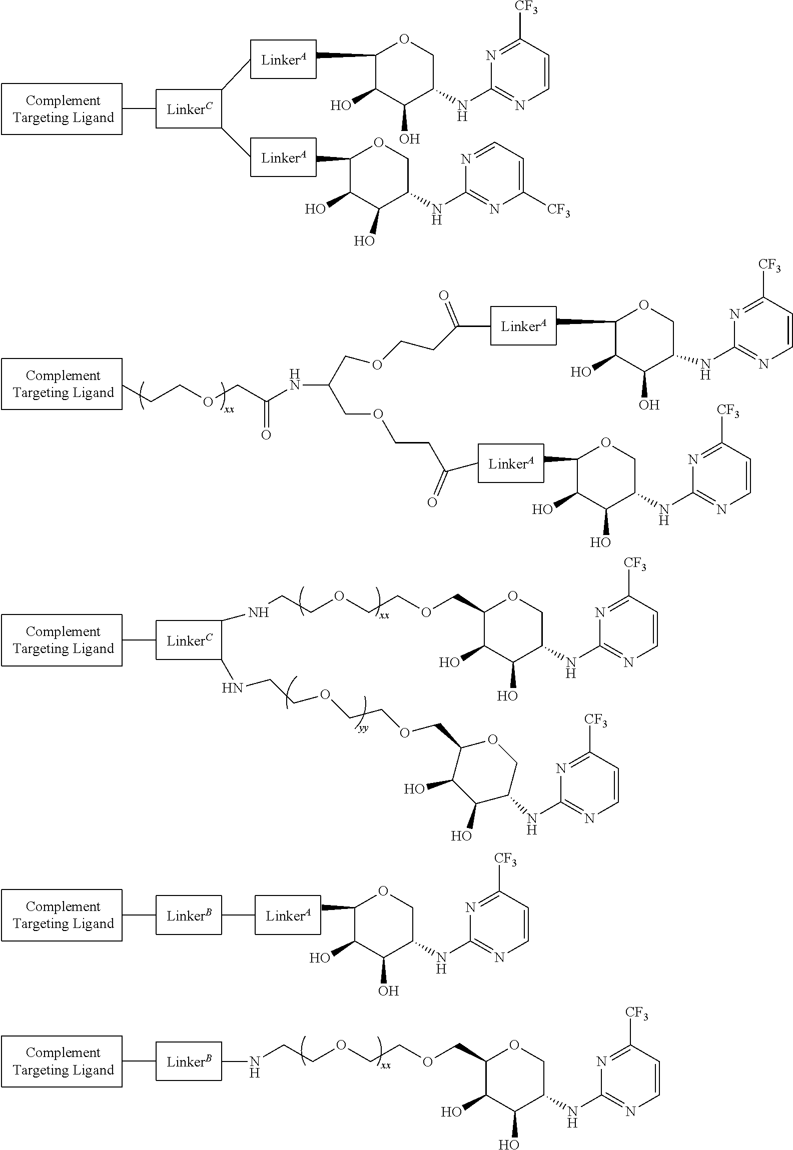

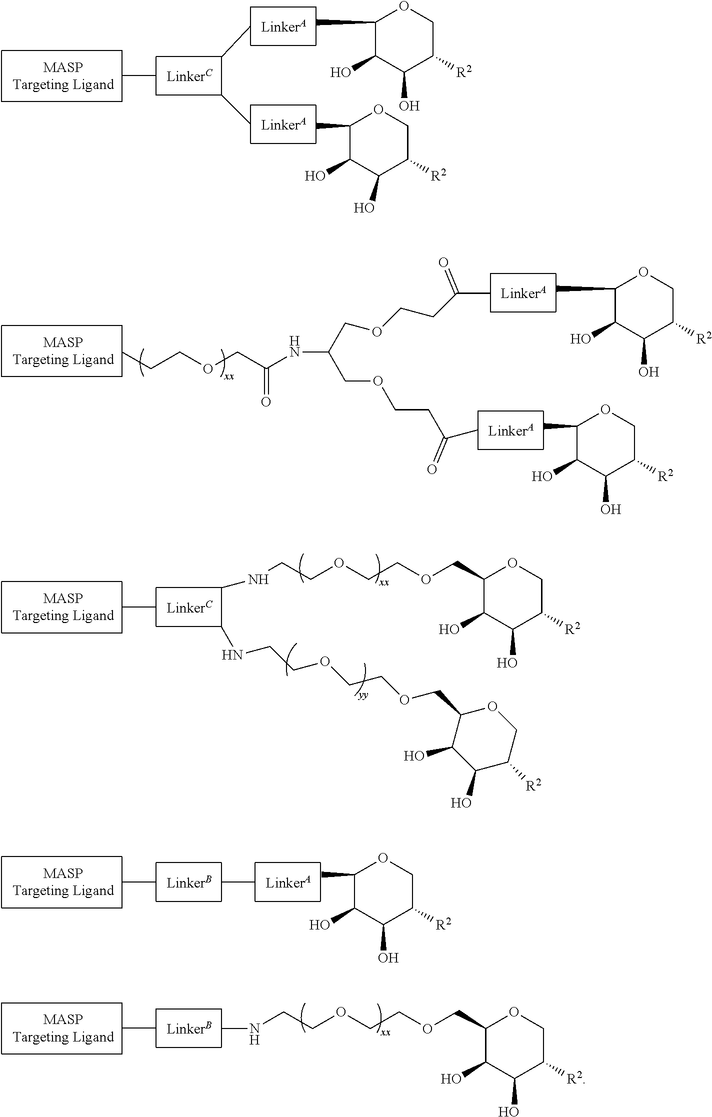

wherein in certain embodiments R² is selected from —NR⁶COR¹⁰, —NR⁶—(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

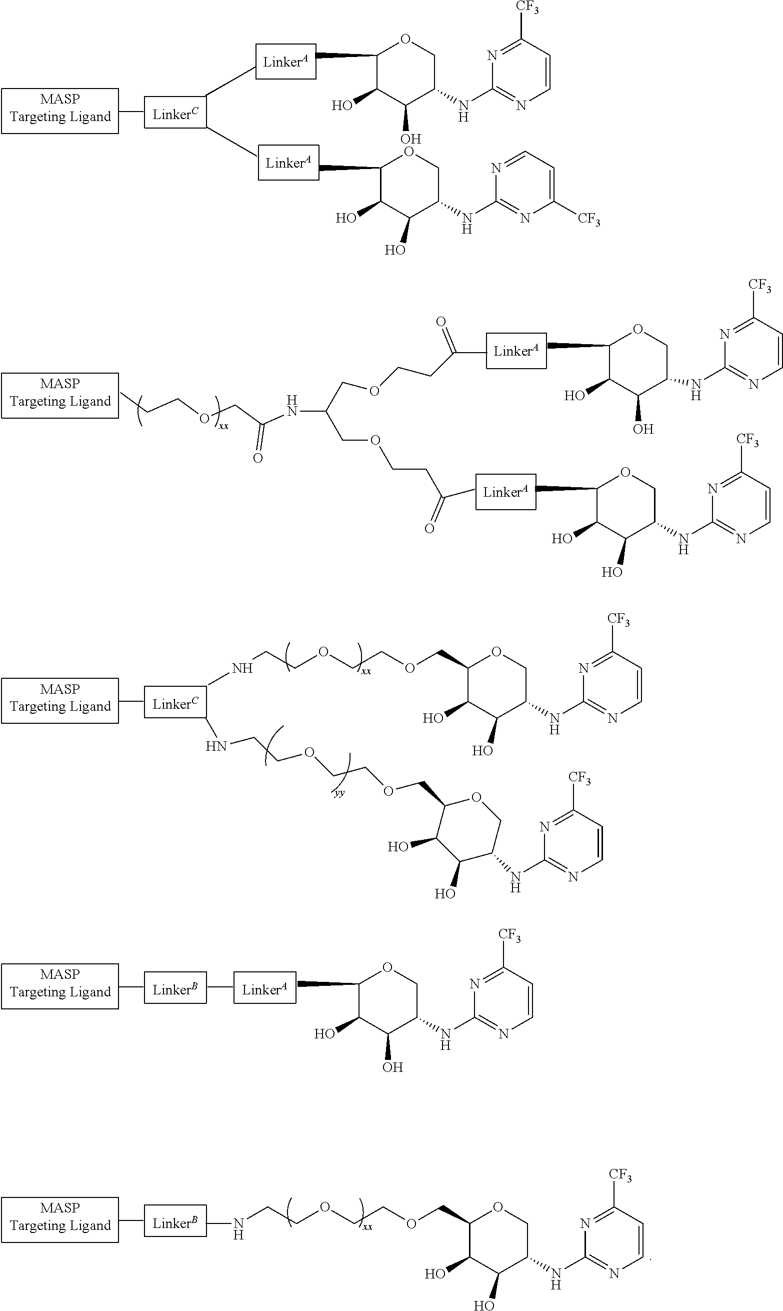

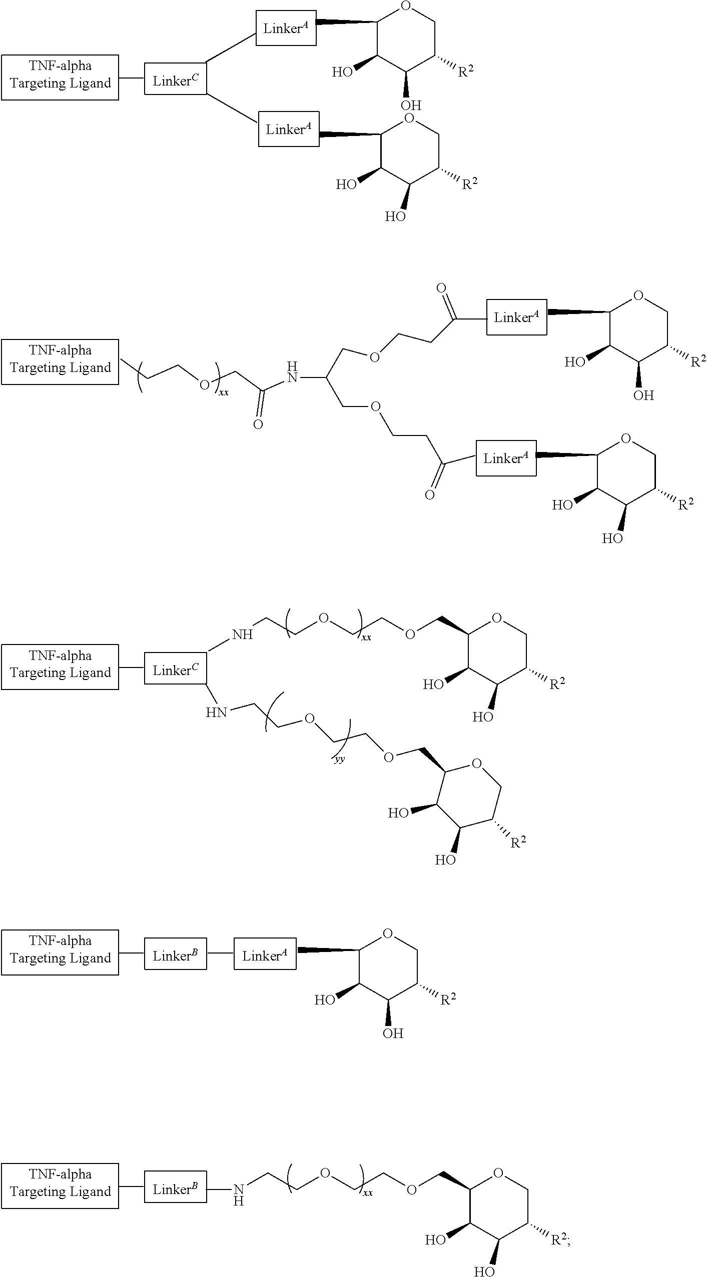

wherein in certain embodiments R² is selected from —NR⁶COR¹⁰, —NR⁶ (5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

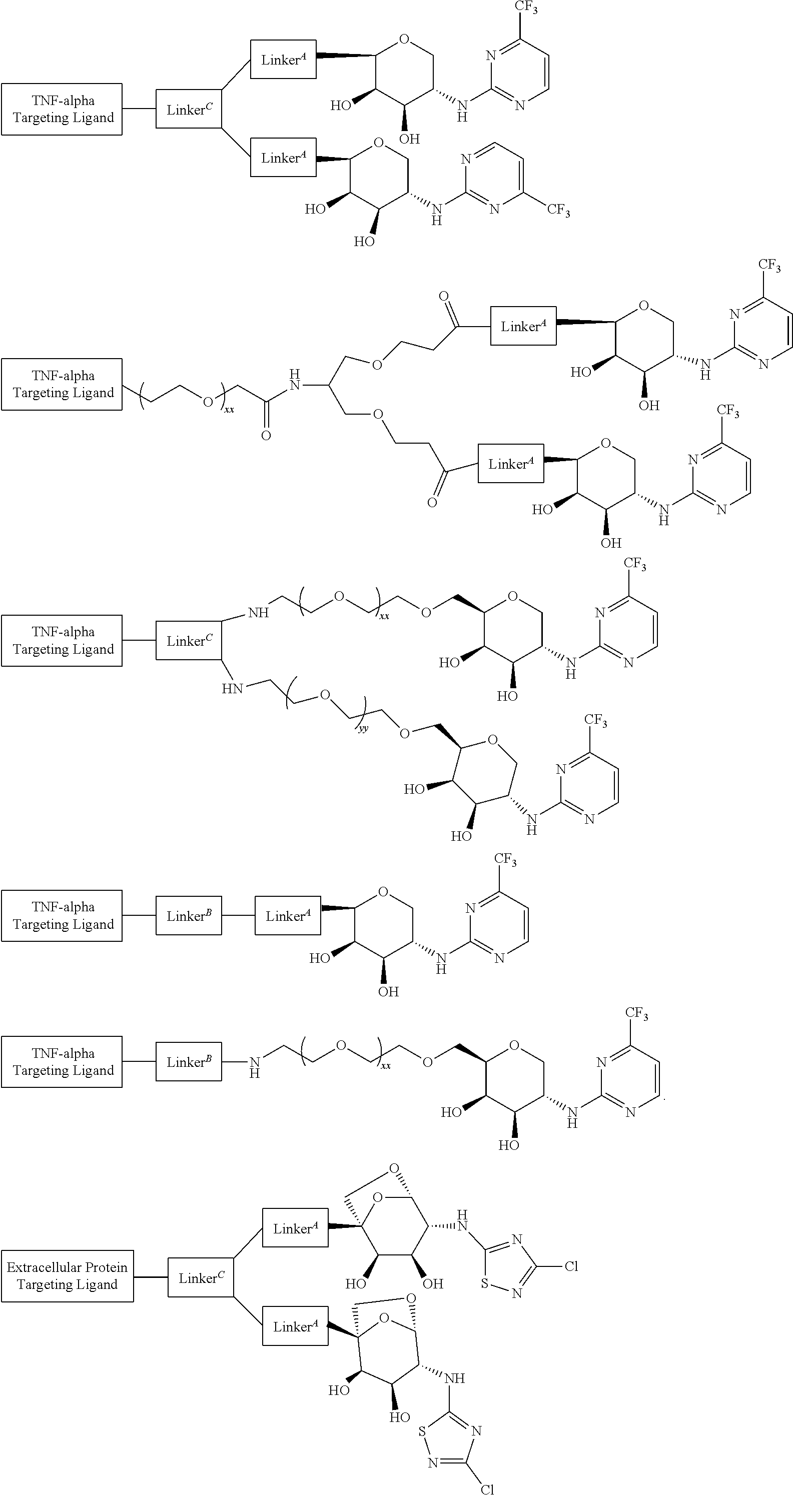

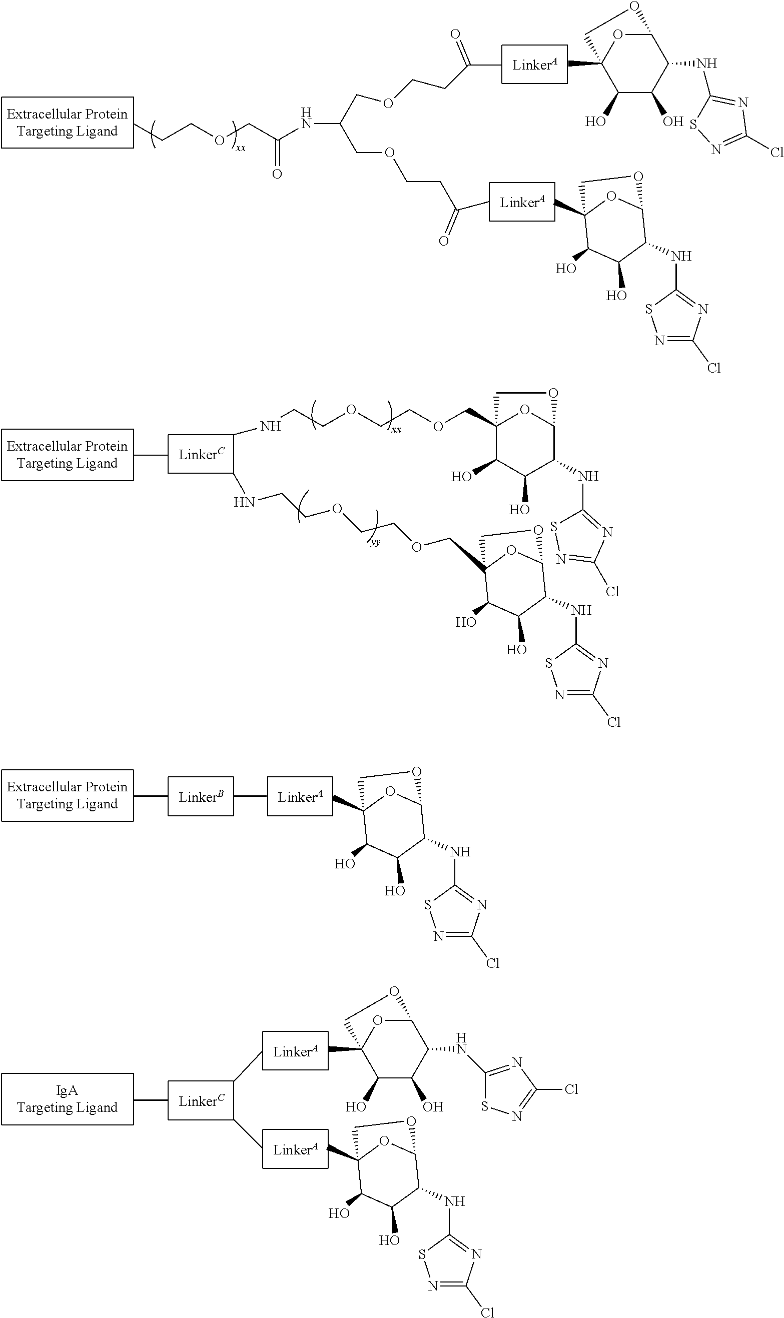

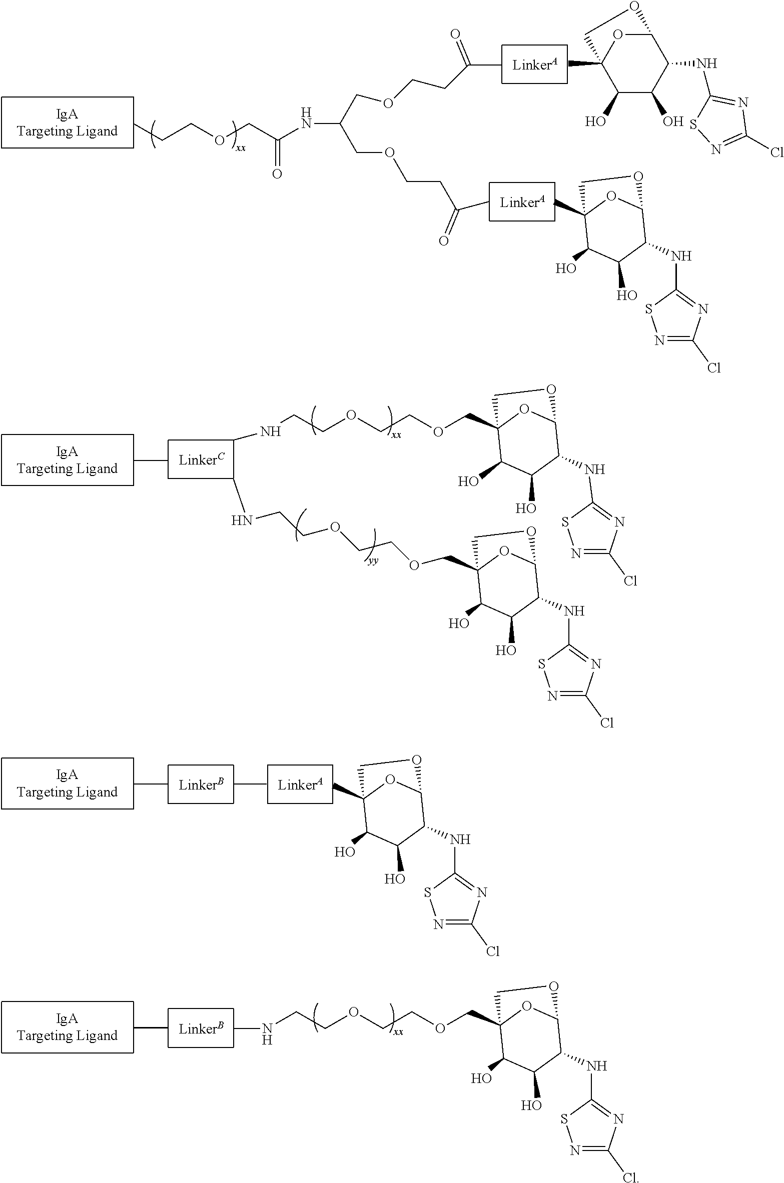

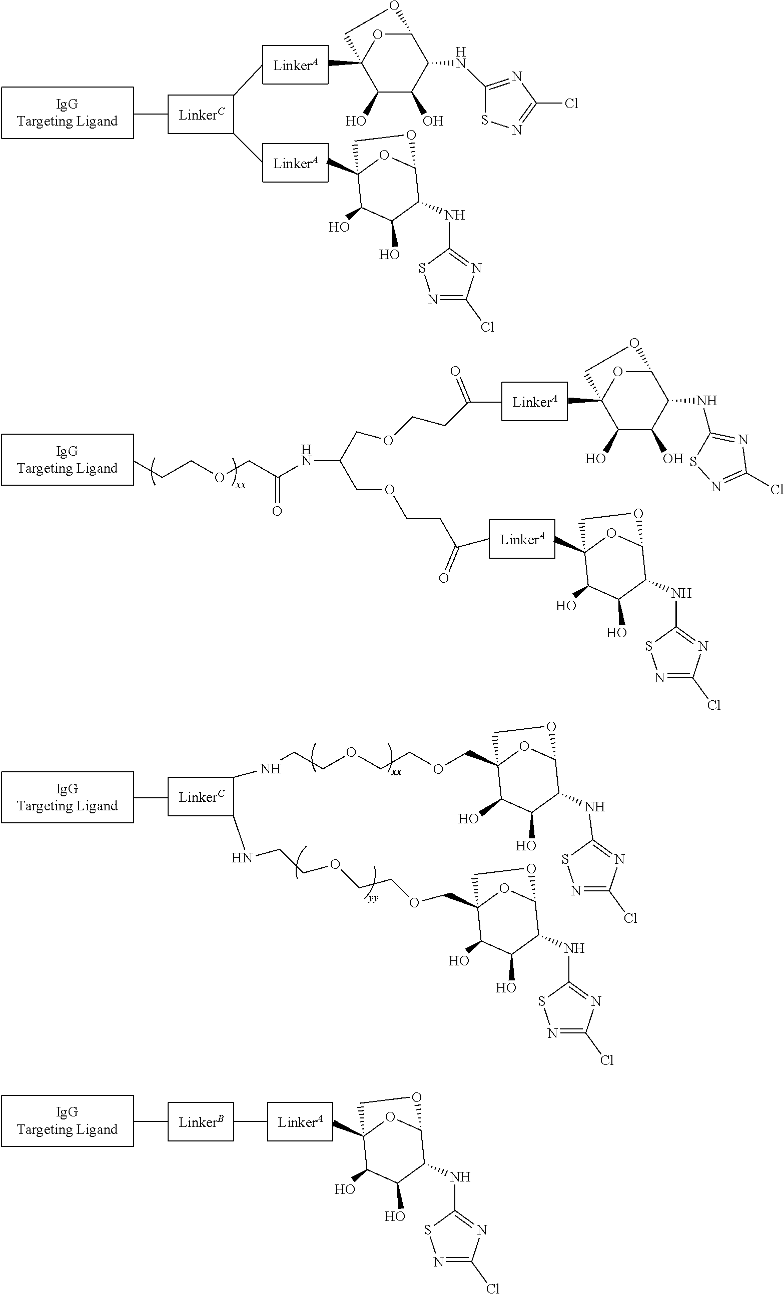

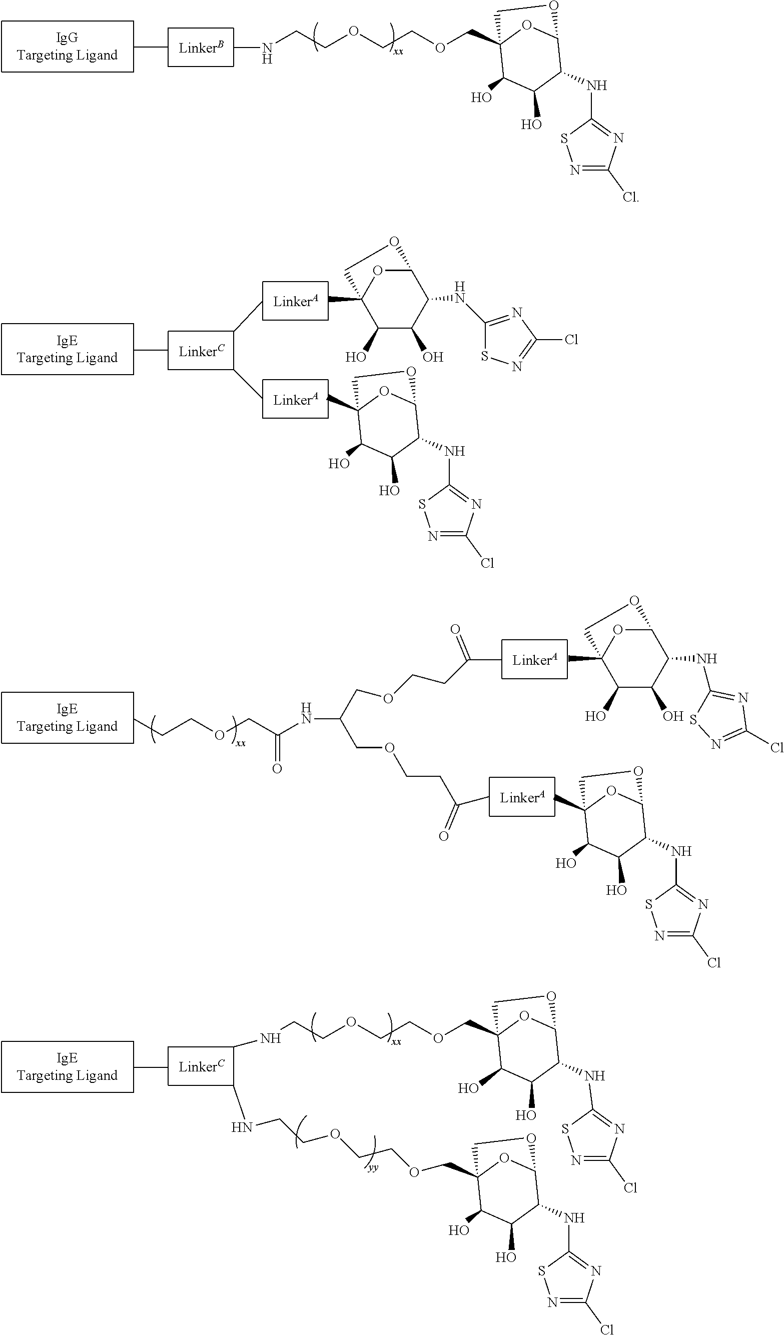

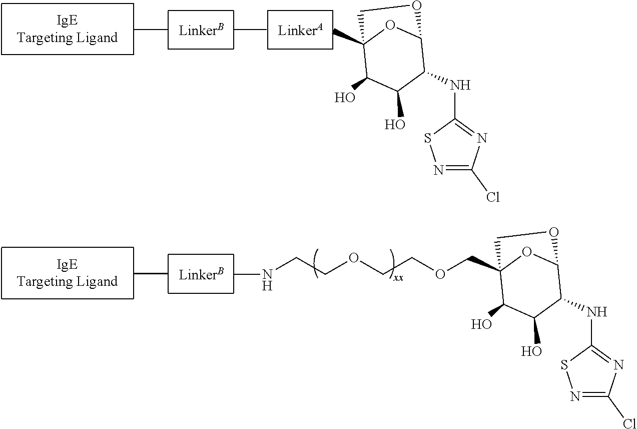

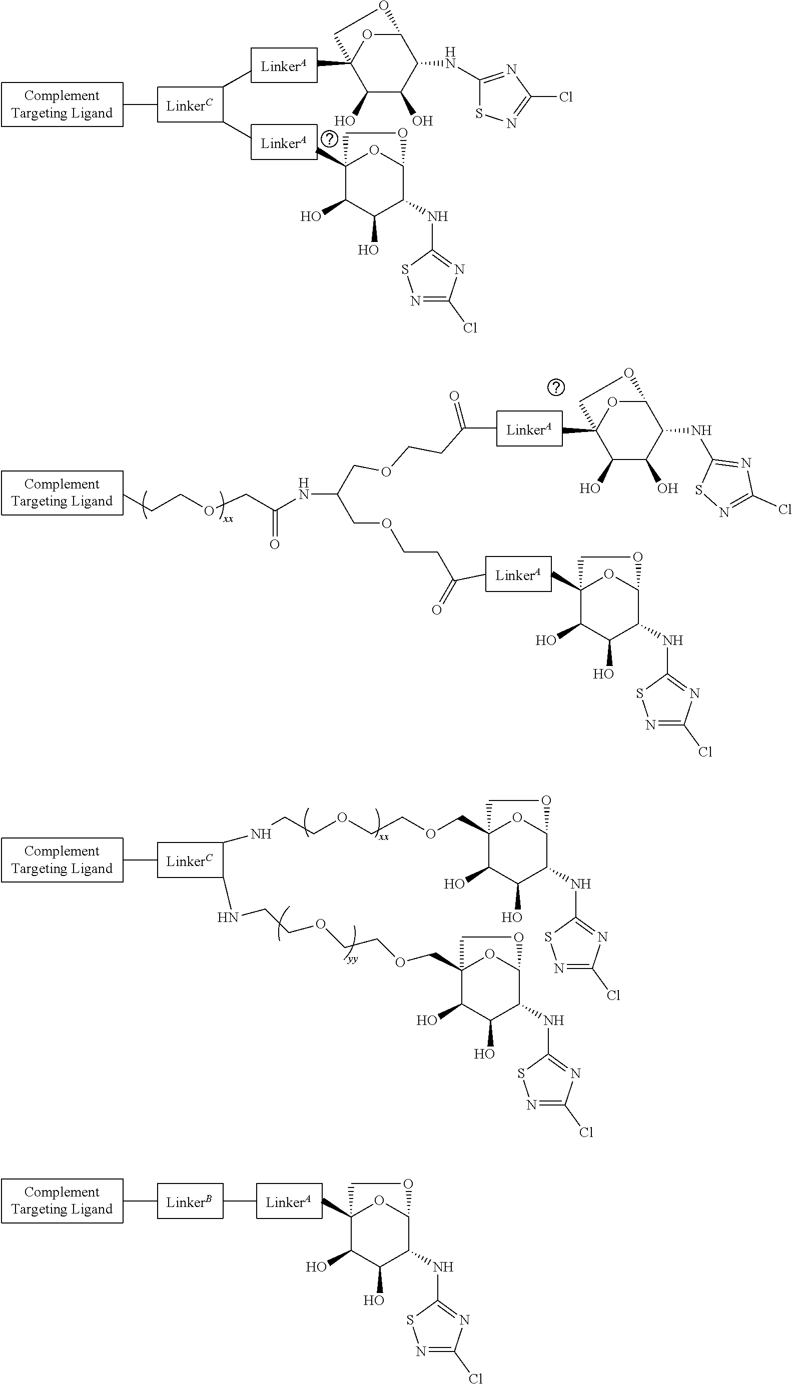

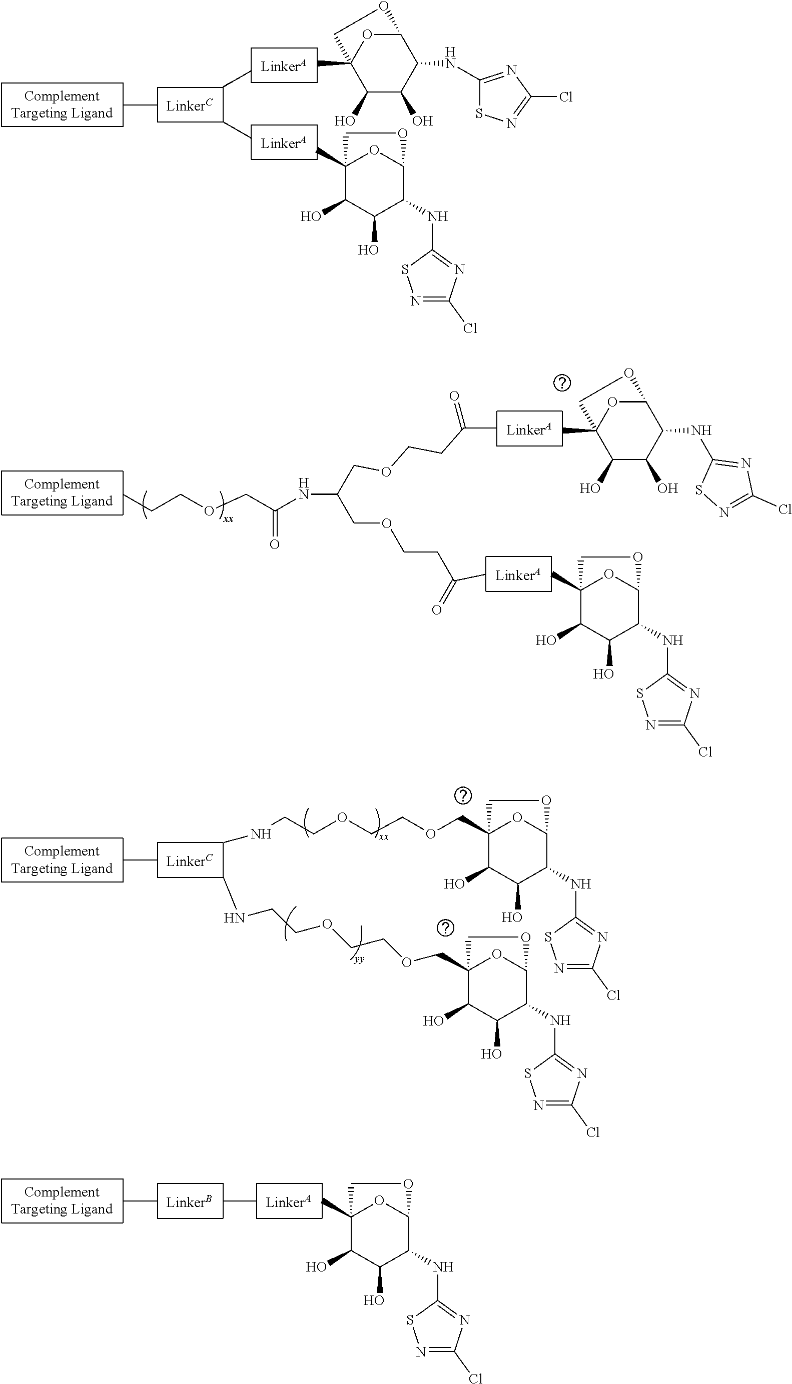

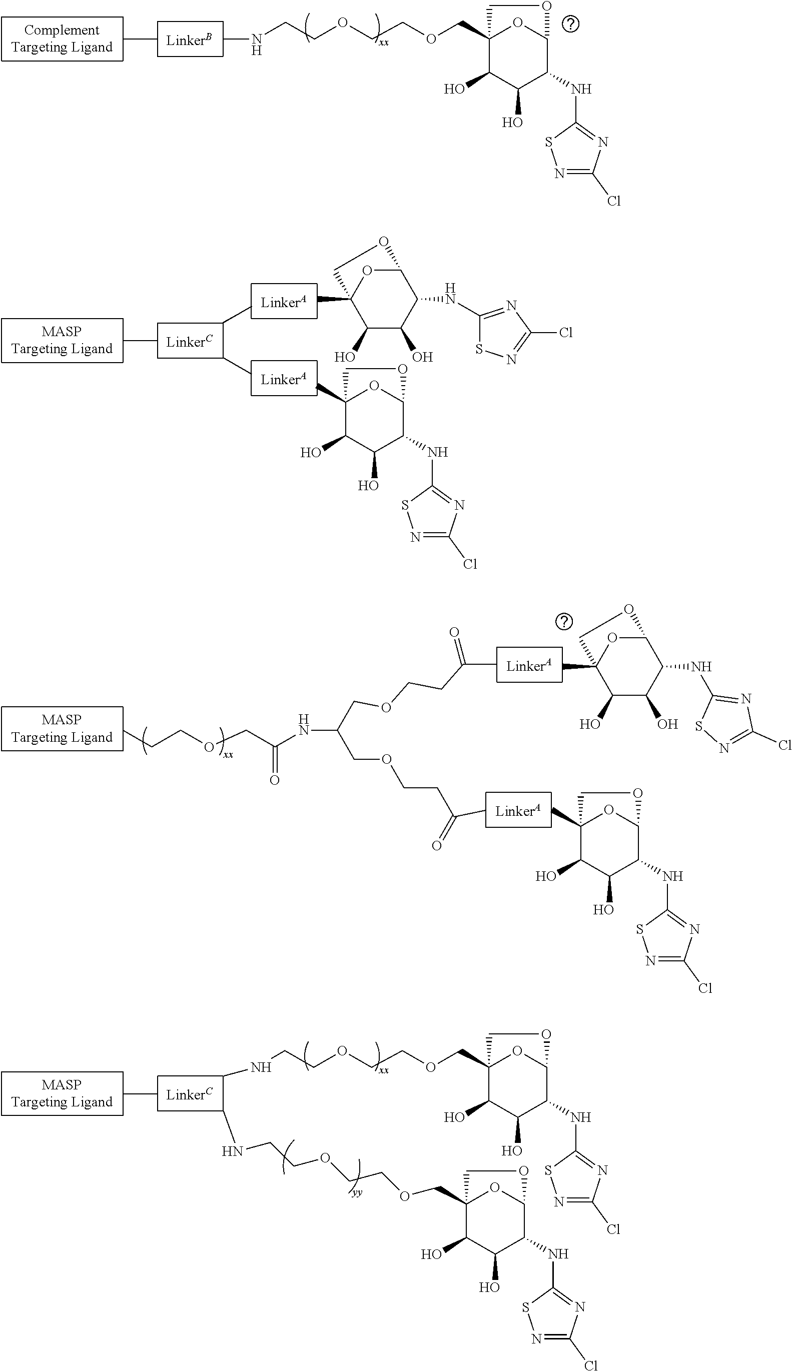

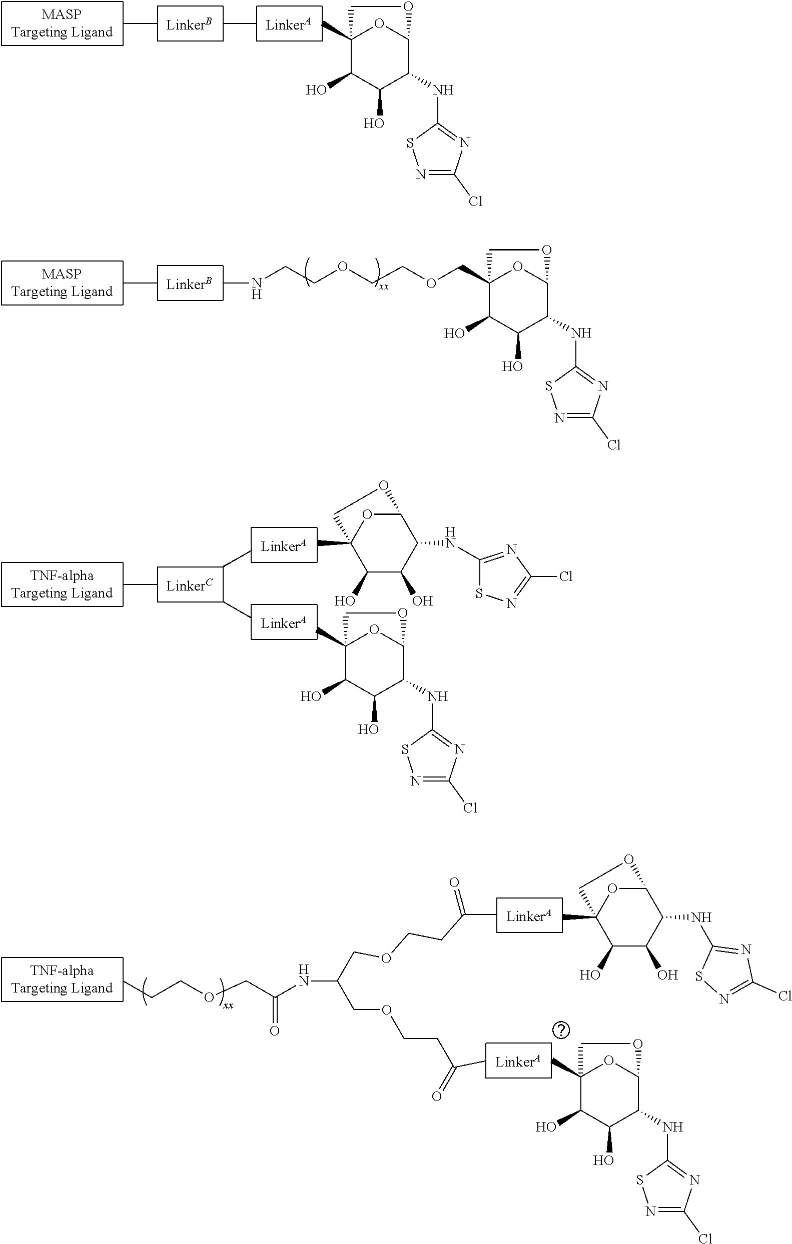

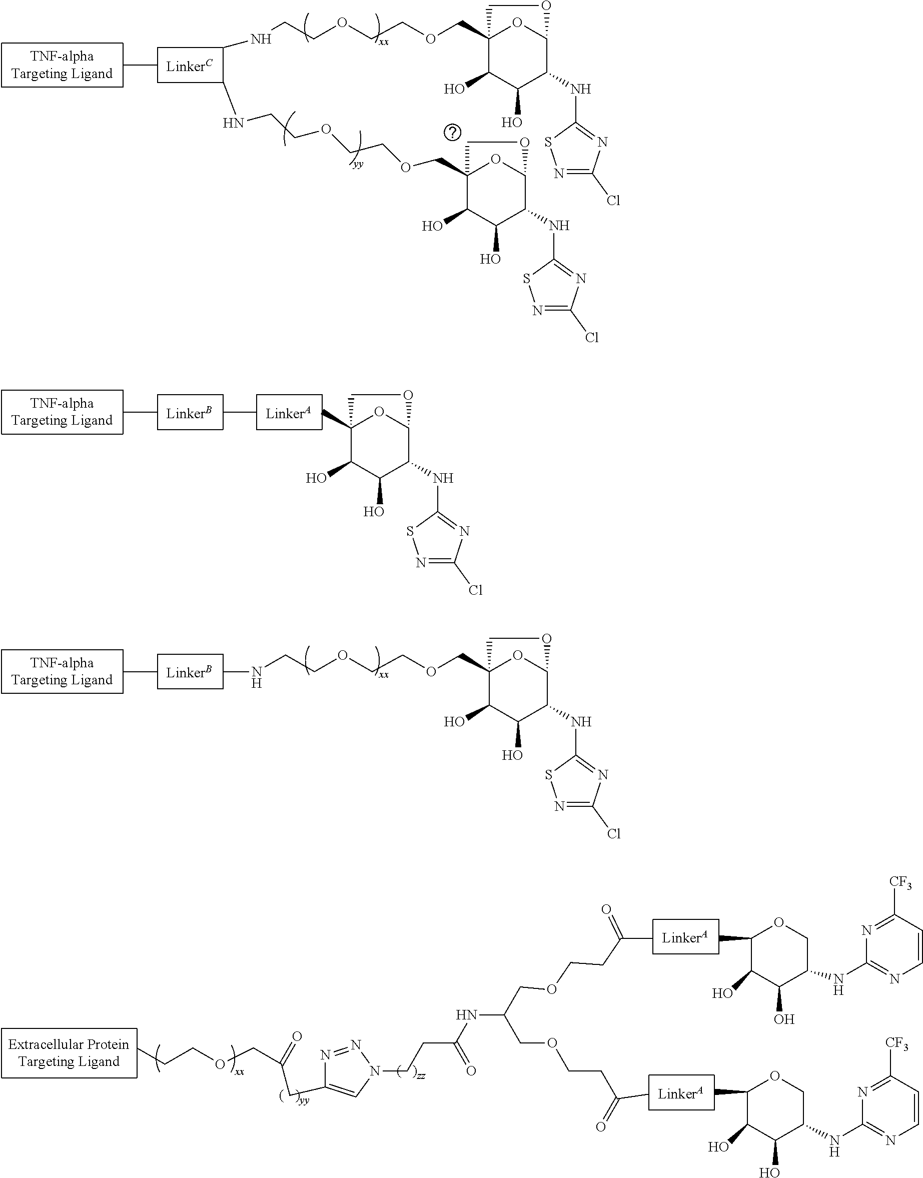

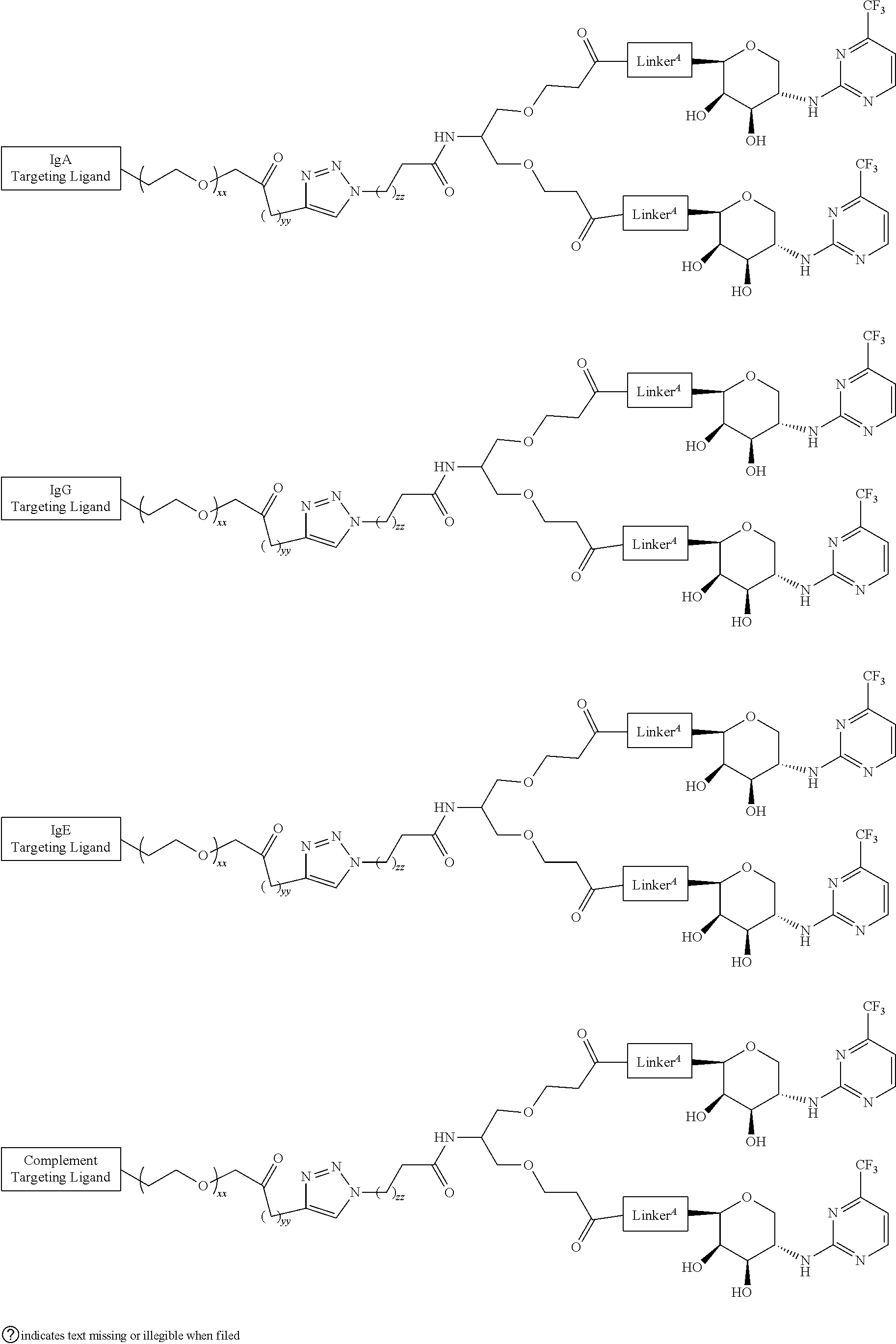

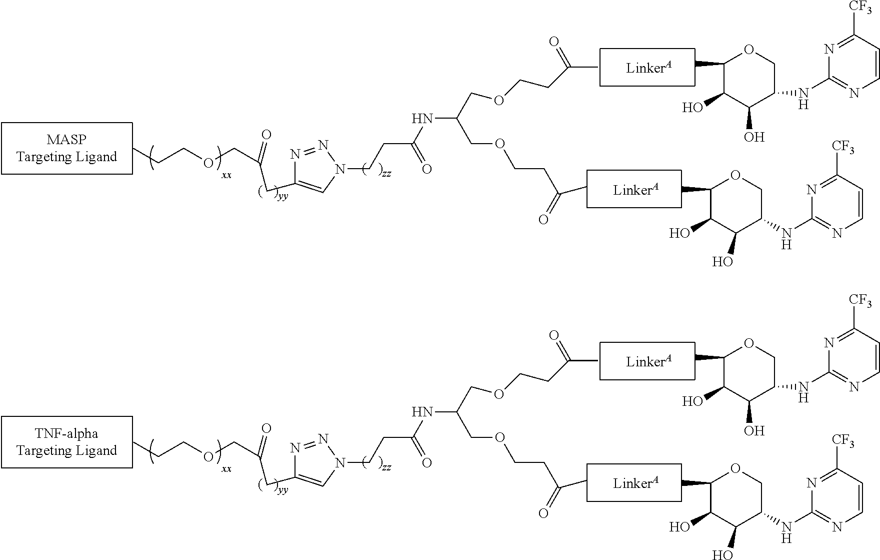

In certain embodiments, an ASGPR ligand useful for incorporation into a compound of Formula II is selected from:

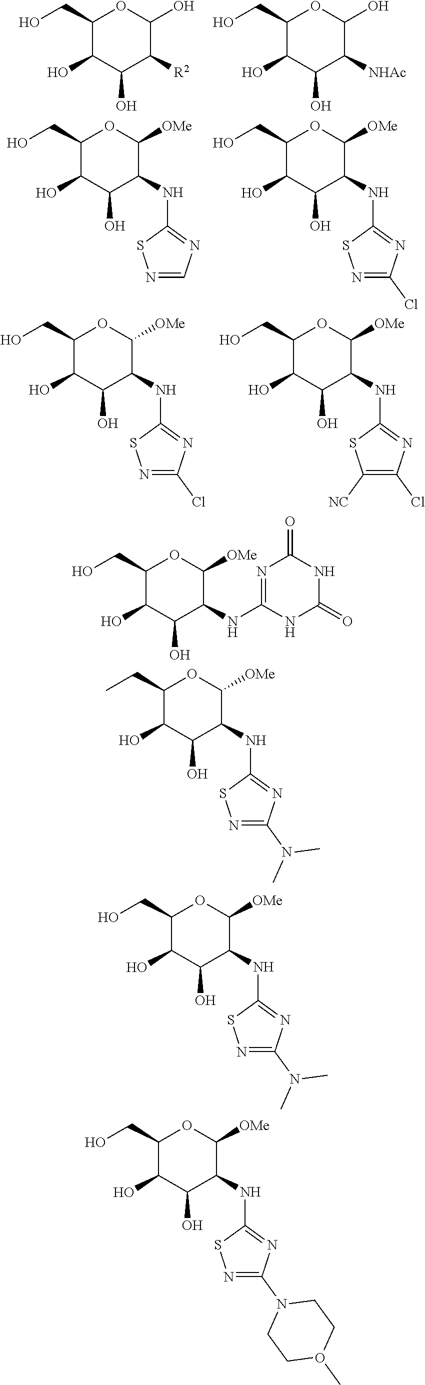

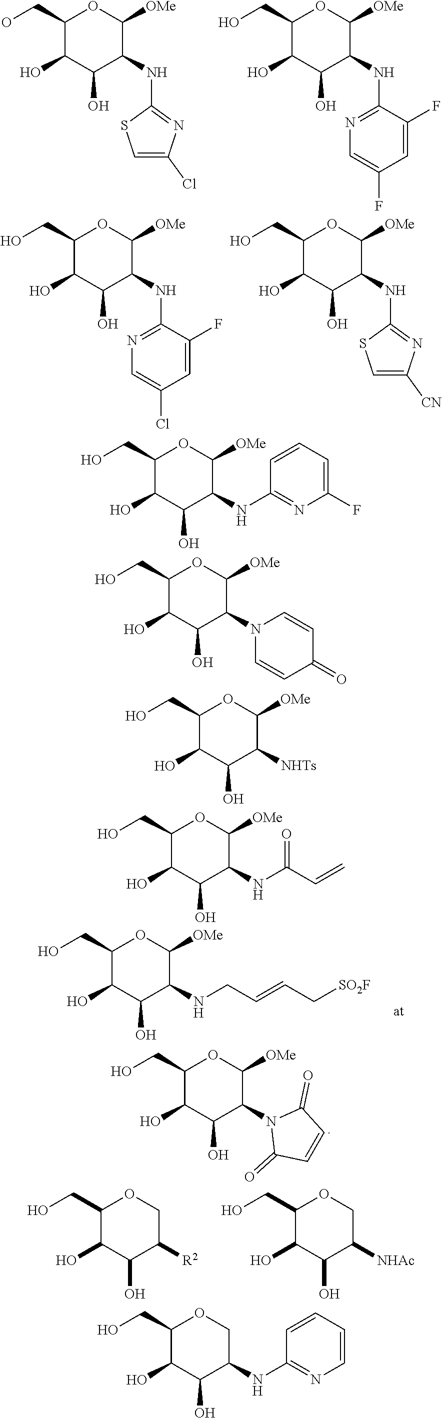

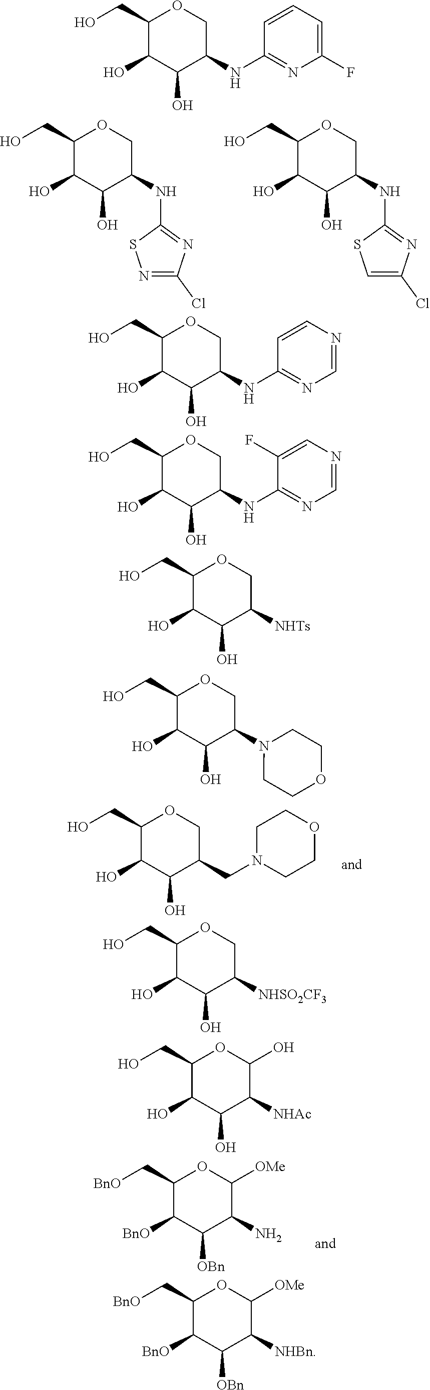

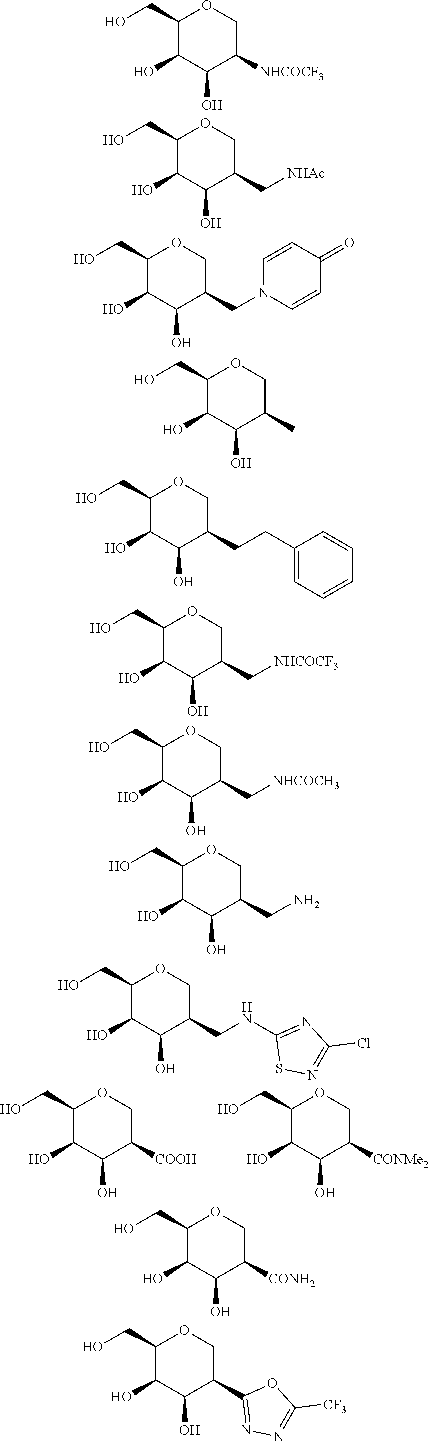

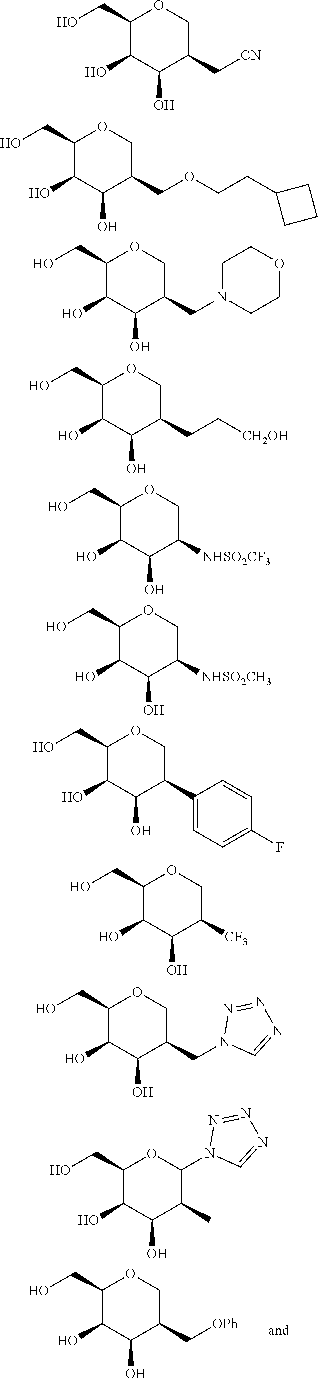

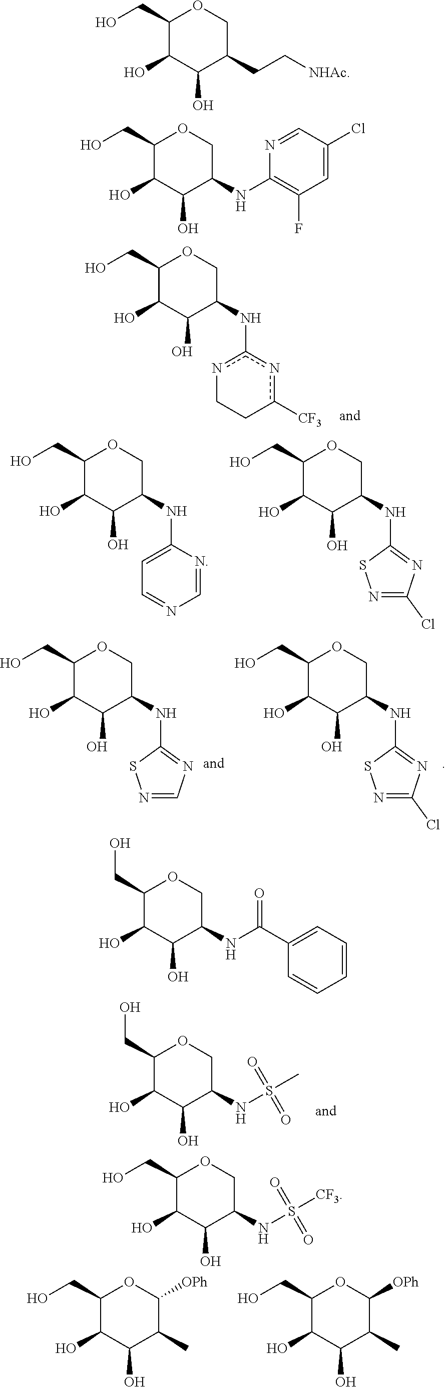

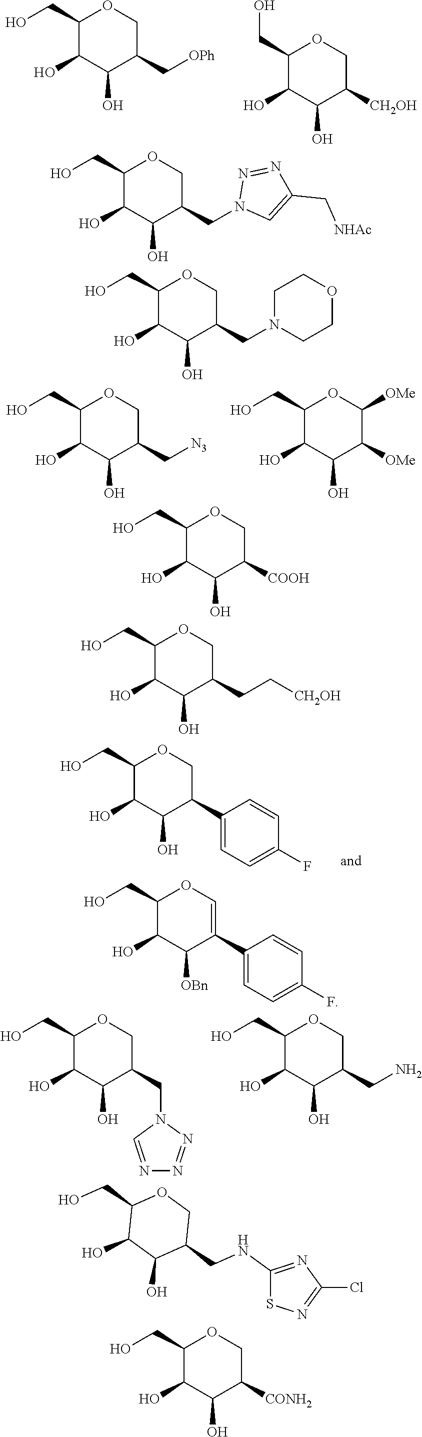

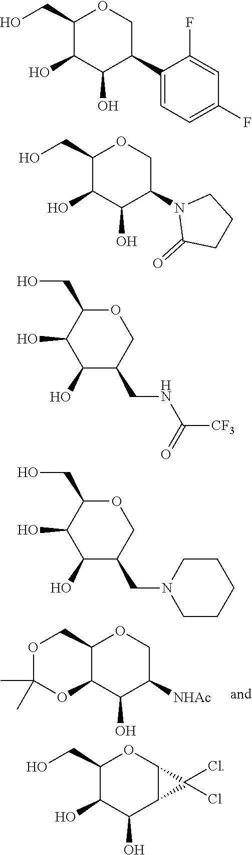

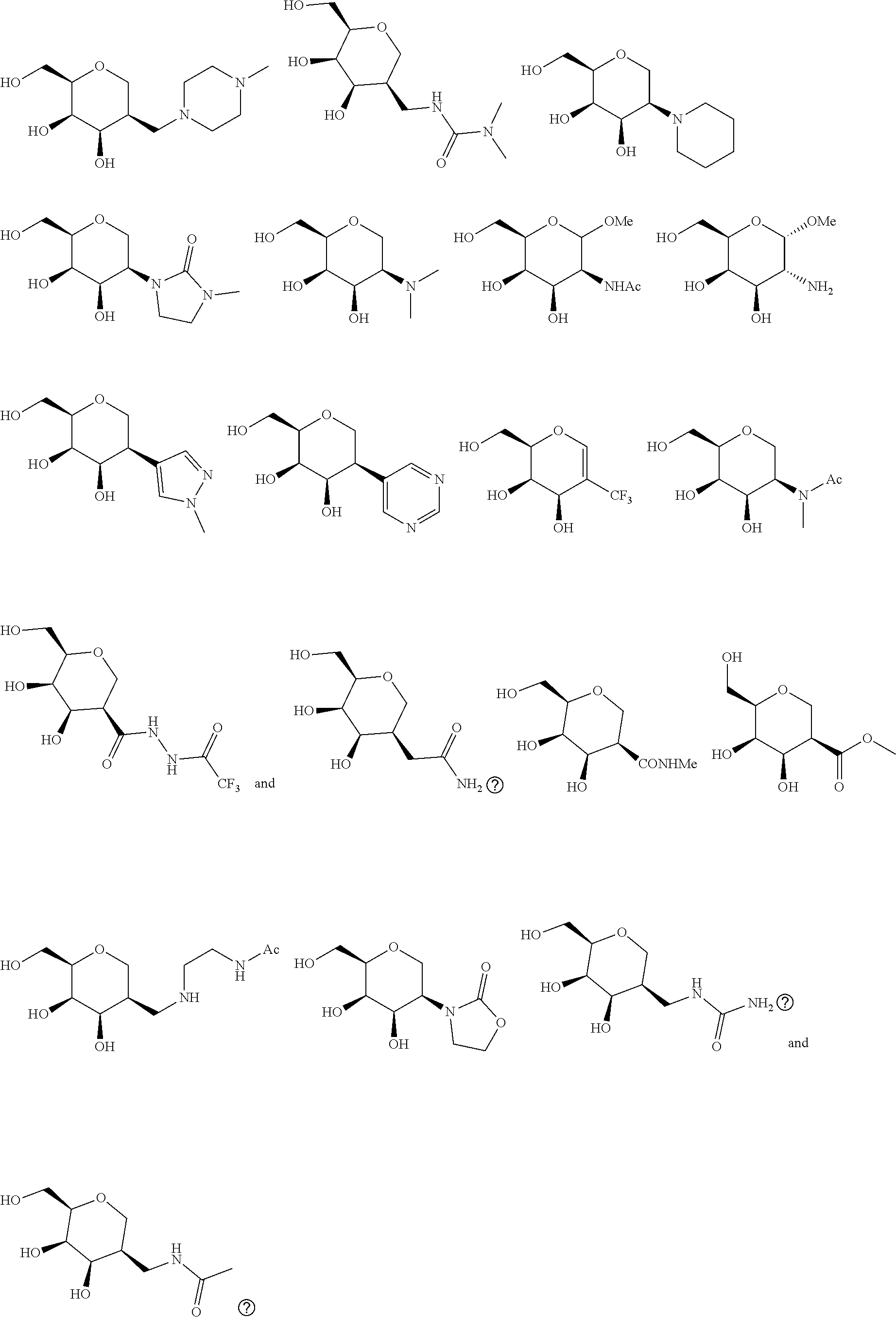

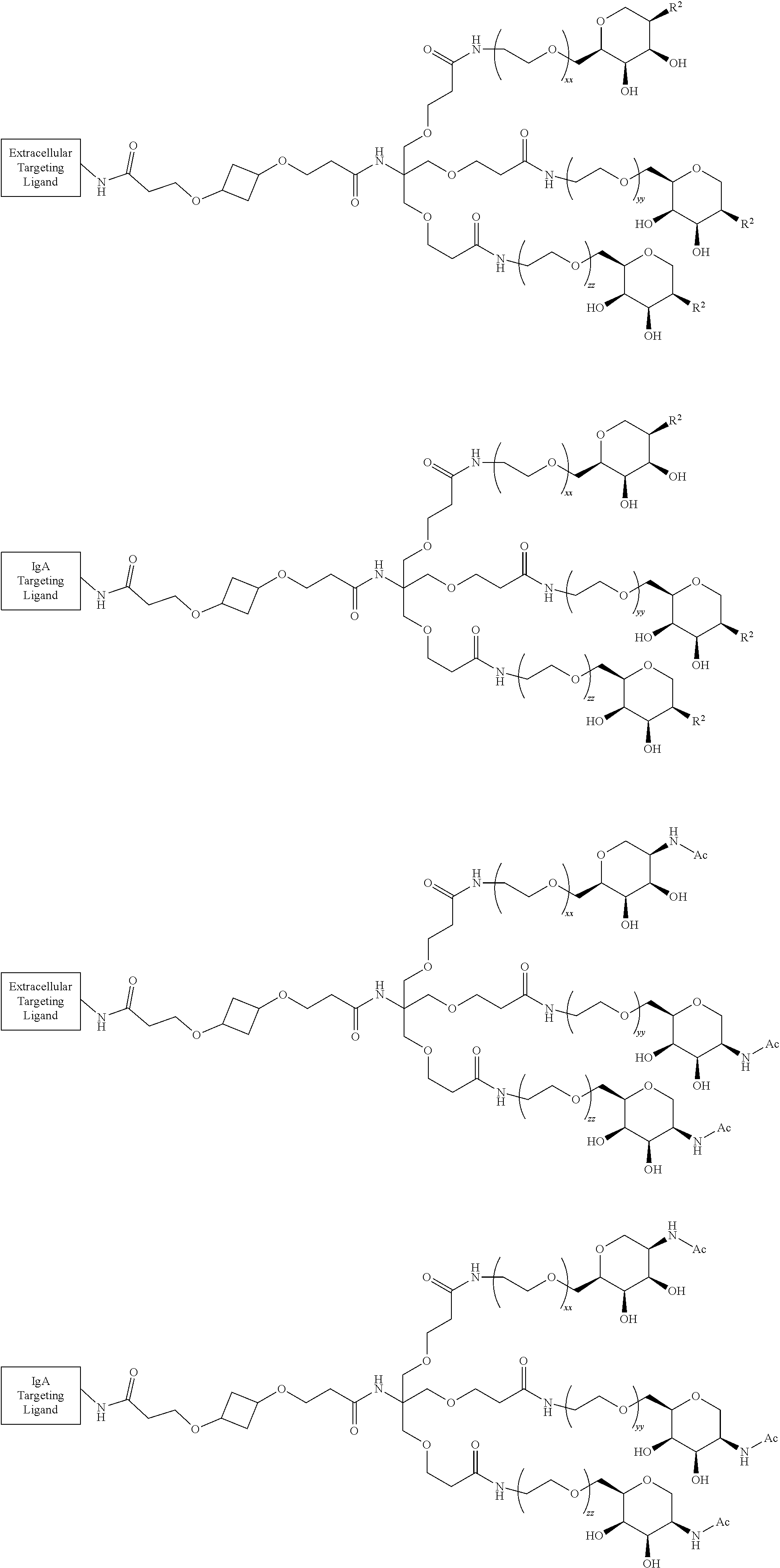

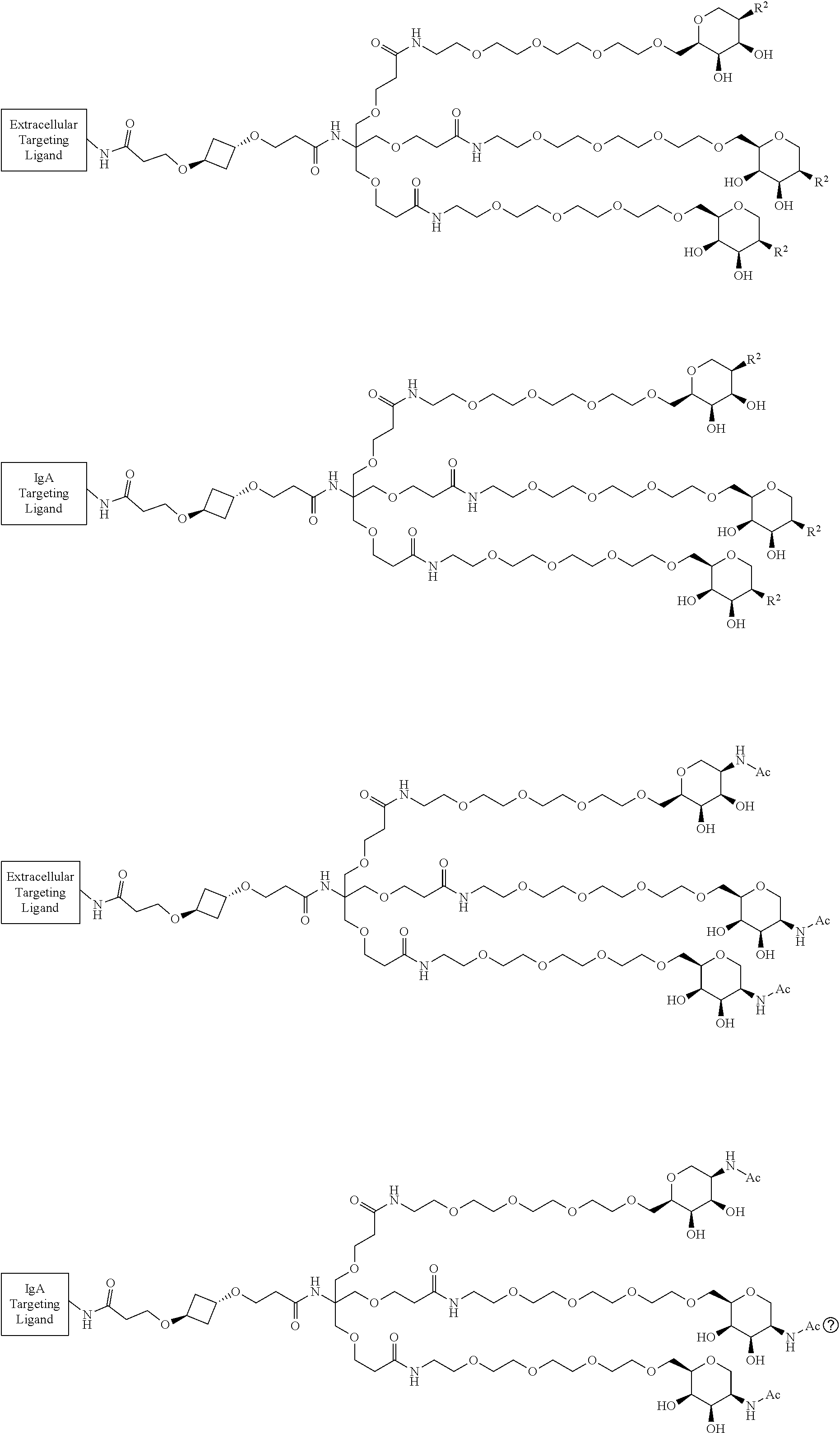

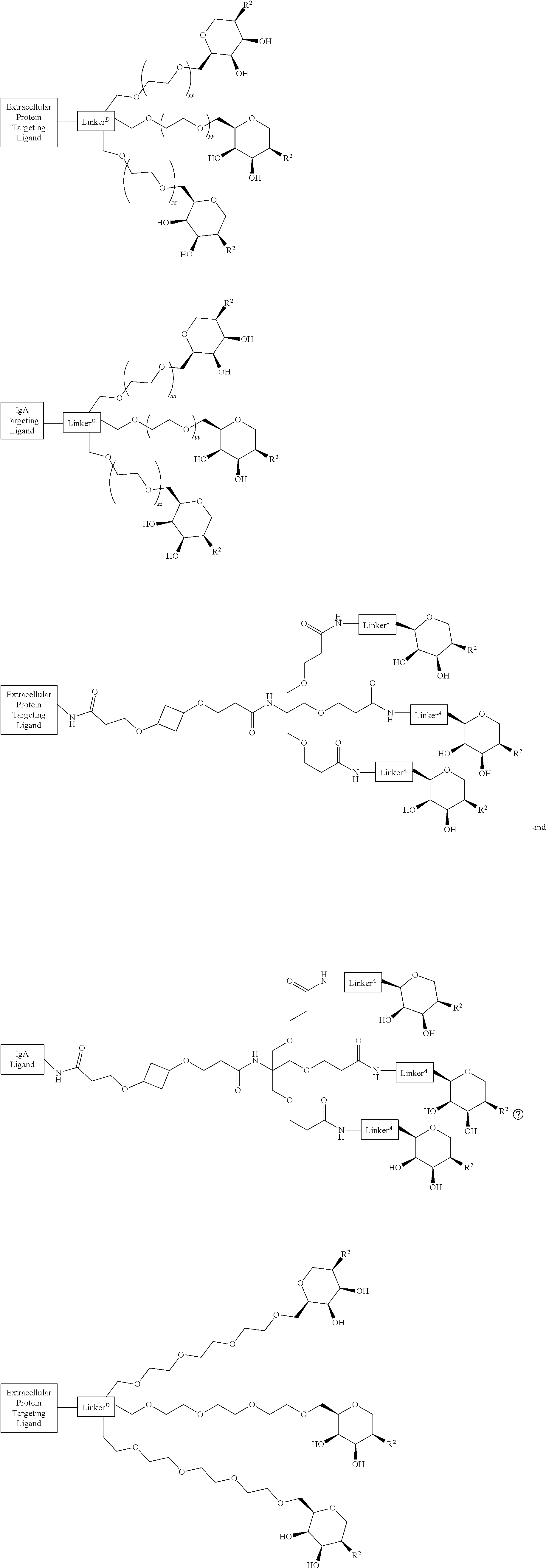

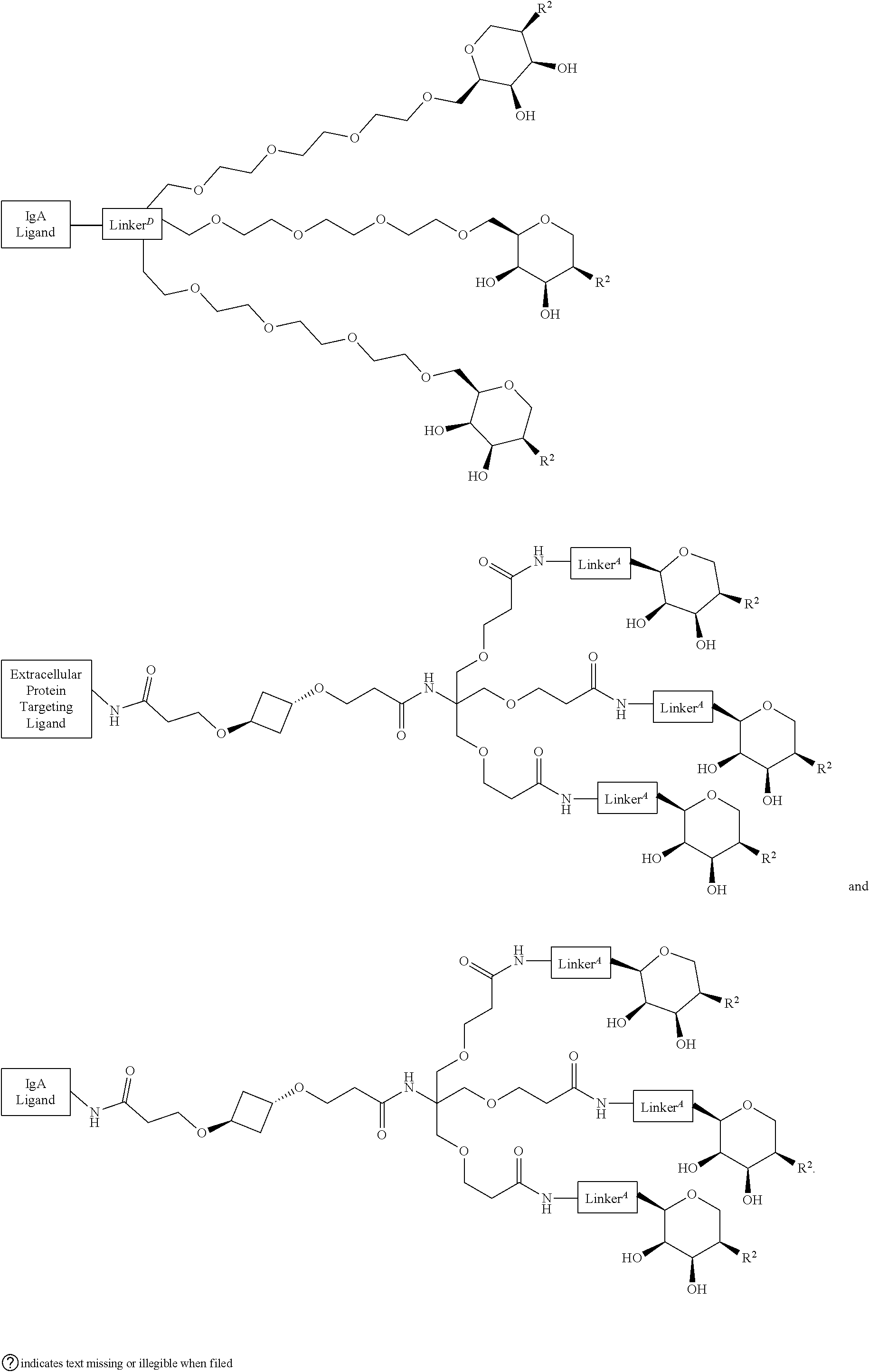

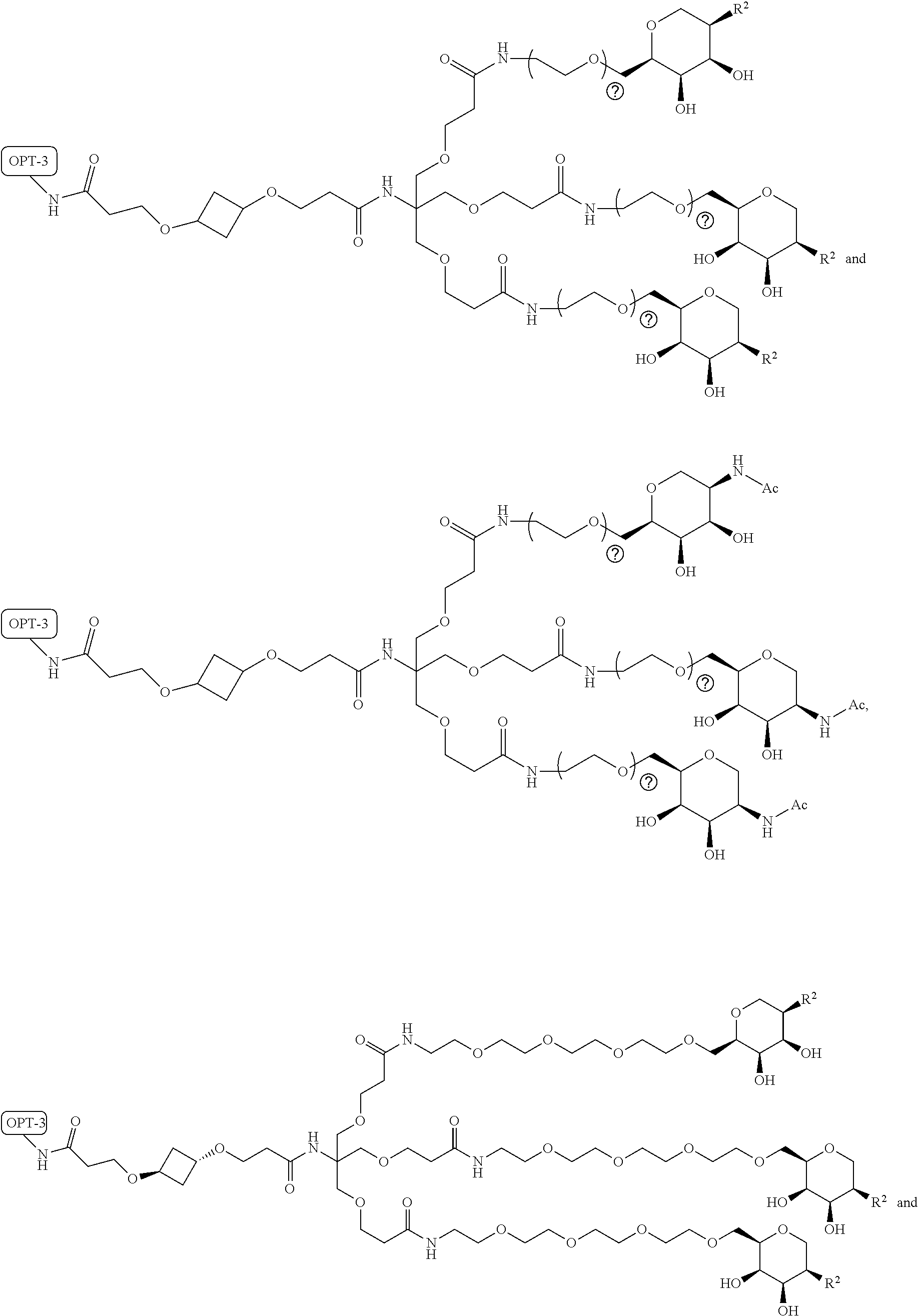

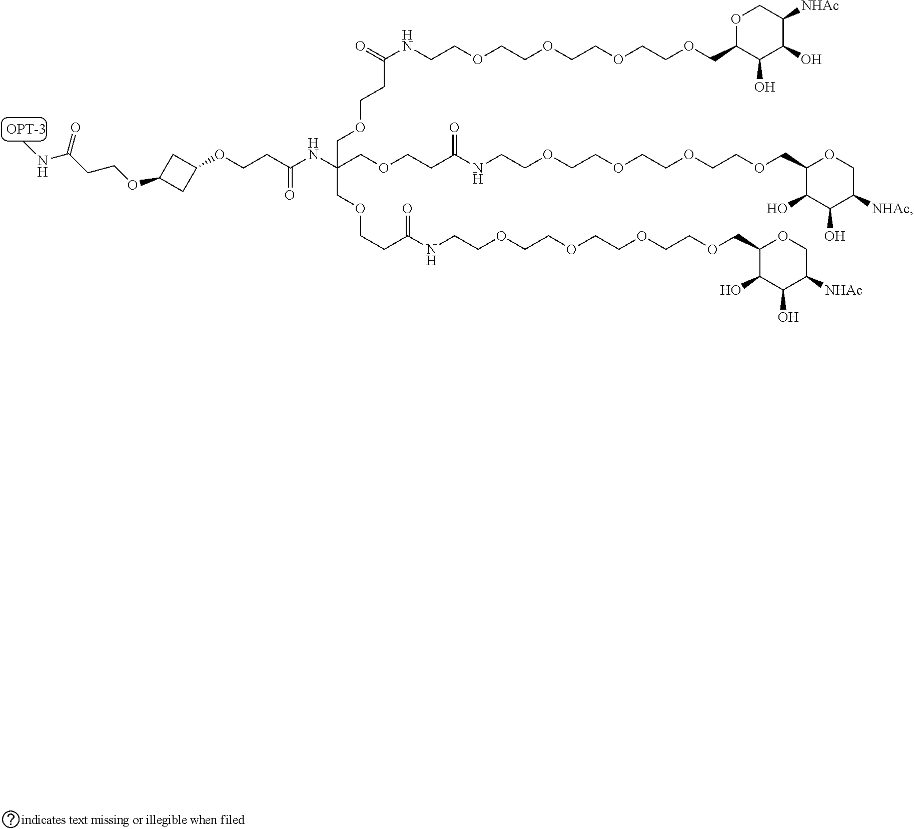 [SMILES: "OPT-3" ; CNC(=O)CCO[C@H]1C[C@H](OCCC(=O)NC(COCCC(=O)NCCOCCOCCOCCOC[C@H]2OC[C@@H](NC(C)=O)[C@@H](O)[C@H]2O)(COCCC(=O)NCCOCCOCCOCCOC[C@H]2OC[C@@H](NC(C)=O)[C@@H](O)[C@H]2O)COCCC(=O)NCCOCCOCCOCCOC[C@H]2OC[C@@H](NC(C)=O)[C@@H](O)[C@H]2O)C1]

### C. The ASGPR Ligand/Binding Moiety in Compounds of Formula II

In certain embodiments, in the compound of Formula II, R¹ is hydrogen.

In certain embodiments, in the compound of Formula II, R¹ is

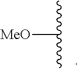 [SMILES: *OC]

In certain embodiments, in the compound of Formula II, R¹ is

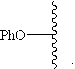 [SMILES: *Oc1ccccc1]

In certain embodiments, in the compound of Formula II, R¹ is

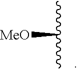

In certain embodiments, in the compound of Formula II, R¹ is

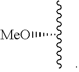

In certain embodiments, in the compound of Formula II, R¹ is

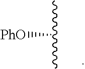

In certain embodiments, in the compound of Formula II, R¹ is

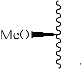

In certain embodiments, in the compound of Formula II, R¹ is C₀-C₆alkyl-cyano optionally substituted with 1, 2, 3, or 4 substituents.

In certain embodiments, in the compound of Formula II, R¹ is alkyl optionally substituted with 1, 2, 3, or 4 substituents.

In certain embodiments, in the compound of Formula II, R¹ is alkenyl optionally substituted with 1, 2, 3, or 4 substituents. In certain embodiments, in the compound of Formula II, R¹ is alkynyl optionally substituted with 1, 2, 3, or 4 substituents. In certain embodiments, in the compound of Formula II, R′ is haloalkyl optionally substituted with 1, 2, 3, or 4 substituents. In certain embodiments, in the compound of Formula II, R¹ is F.

In certain embodiments, in the compound of Formula II, R¹ is CL.

In certain embodiments, in the compound of Formula II, R¹ is Br.

In certain embodiments, in the compound of Formula II, R¹ is aryl optionally substituted with 1, 2, 3, or 4 substituents.

In certain embodiments, in the compound of Formula II, R¹ is arylalkyl optionally substituted with 1, 2, 3, or 4 substituents.

In certain embodiments, in the compound of Formula II, R¹ is heteroaryl optionally substituted with 1, 2, 3, or 4 substituents.

In certain embodiments, in the compound of Formula II, R′ is heteroaryl alkyl optionally substituted with 1, 2, 3, or 4 substituents.

In certain embodiments, in the compound of Formula II, R′ is heterocycle optionally substituted with 1, 2, 3, or 4 substituents.

In certain embodiments, in the compound of Formula II, R′ is heterocycloalkyl optionally substituted with 1, 2, 3, or 4 substituents.

In certain embodiments, in the compound of Formula II, R′ is haloalkoxy optionally substituted with 1, 2, 3, or 4 substituents.

In certain embodiments, in the compound of Formula II, R¹ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

In certain embodiments, in the compound of Formula II, R² is aryl optionally substituted with 1, 2, 3, or 4 substituents.

In certain embodiments, in the compound of Formula II, R² is heterocycle optionally substituted with 1, 2, 3, or 4 substituents.

In certain embodiments, in the compound of Formula II, R² is heteroaryl containing 1 or 2 heteroatoms independently selected from N, O, and S optionally substituted with 1, 2, 3, or 4 substituents.

In certain embodiments, in the compound of Formula II, R² is selected from

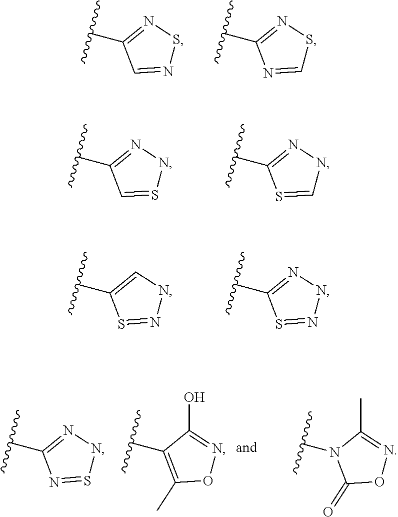

In certain embodiments, in the compound of Formula II, R² is heterocycle optionally substituted with 1, 2, 3, or 4 substituents.

In certain embodiments, in the compound of Formula II, R² is —NR⁸—S(O)—R³ optionally substituted with 1, 2, 3, or 4 substituents.

In certain embodiments, in the compound of Formula II, R² is —NR⁸—C(S)—R³ optionally substituted with 1, 2, 3, or 4 substituents.

In certain embodiments, in the compound of Formula II, R² is —NR⁸—S(O)(NR⁶)—R³ optionally substituted with 1, 2, 3, or 4 substituents.

In certain embodiments, in the compound of Formula II, R² is —N═S(O)(R³)₂ optionally substituted with 1, 2, 3, or 4 substituents.

In certain embodiments, in the compound of Formula II, R² is —NR⁸C(O)NR⁹S(O)₂R³ optionally substituted with 1, 2, 3, or 4 substituents.

In certain embodiments, in the compound of Formula II, R² is —NR⁸—S(O)₂-R¹⁰ optionally substituted with 1, 2, 3, or 4 substituents.

In certain embodiments, in the compound of Formula II, R² is —NR⁸—C(NR⁶)—R³ optionally substituted with 1, 2, 3, or 4 substituents.

In certain embodiments, in the compound of Formula II, R² is hydrogen.

In certain embodiments, in the compound of Formula II, R² is R¹⁰, In certain embodiments, in the compound of Formula II, R² is alkyl-C(O)—R³.

In certain embodiments, in the compound of Formula II, R² is —C(O)—R³.

In certain embodiments, in the compound of Formula II, R² is alkyl.

In certain embodiments, in the compound of Formula II, R² is haloalkyl.

In certain embodiments, in the compound of Formula II, R² is —OC(O)R³.

In certain embodiments, in the compound of Formula II, R² is —NR⁸—C(O)R¹⁰.

In certain embodiments, in the compound of Formula II, R² is alkenyl optionally substituted with 1, 2, 3, or 4 substituents.

In certain embodiments, in the compound of Formula II, R² is allyl optionally substituted with 1, 2, 3, or 4 substituents.

In certain embodiments, in the compound of Formula II, R² is alkynyl optionally substituted with 1, 2, 3, or 4 substituents.

In certain embodiments, in the compound of Formula II, R² is —NR⁶-alkenyl optionally substituted with 1, 2, 3, or 4 substituents.

In certain embodiments, in the compound of Formula II, R² is —O-alkenyl optionally substituted with 1, 2, 3, or 4 substituents.

In certain embodiments, in the compound of Formula II, R² is —NR⁶-alkynyl optionally substituted with 1, 2, 3, or 4 substituents.

In certain embodiments, in the compound of Formula II, R² is —NR⁶-heteroaryl optionally substituted with 1, 2, 3, or 4 substituents.

In certain embodiments, in the compound of Formula II, R² is —NR⁶-aryl optionally substituted with 1, 2, 3, or 4 substituents.

In certain embodiments, in the compound of Formula II, R² is —O-heteroaryl optionally substituted with 1, 2, 3, or 4 substituents.

In certain embodiments, in the compound of Formula II, R² is —O-aryl optionally substituted with 1, 2, 3, or 4 substituents.

In certain embodiments, in the compound of Formula II, R² is —O-alkynyl optionally substituted with 1, 2, 3, or 4 substituents.

In certain embodiments, in the compound of Formula II, R² is selected from and

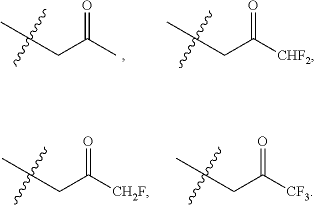

In certain embodiments, in the compound of Formula II, R² is selected from

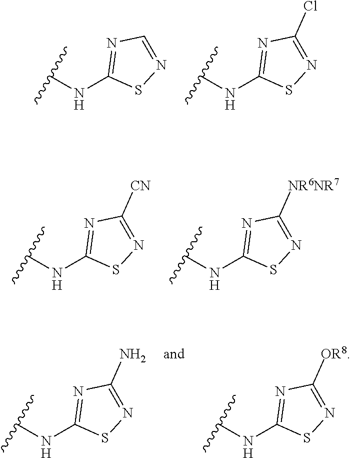

In certain embodiments, in the compound of Formula II, R² is selected from

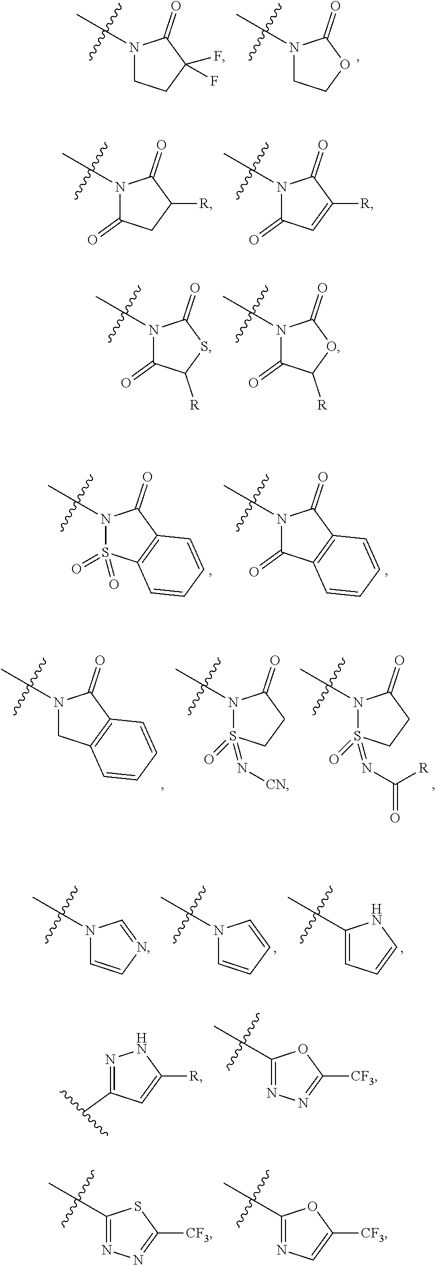

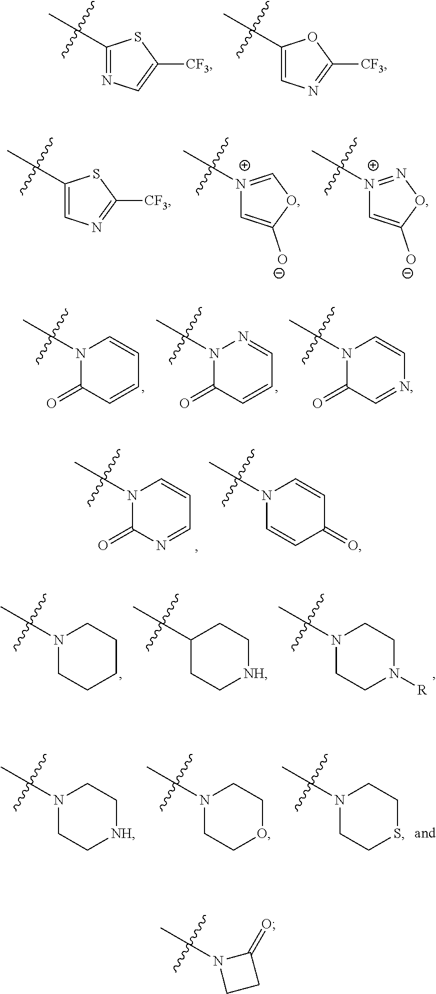

and wherein R is an optional substituent as defined herein.

In certain embodiments, in the compound of Formula II, R² is selected from

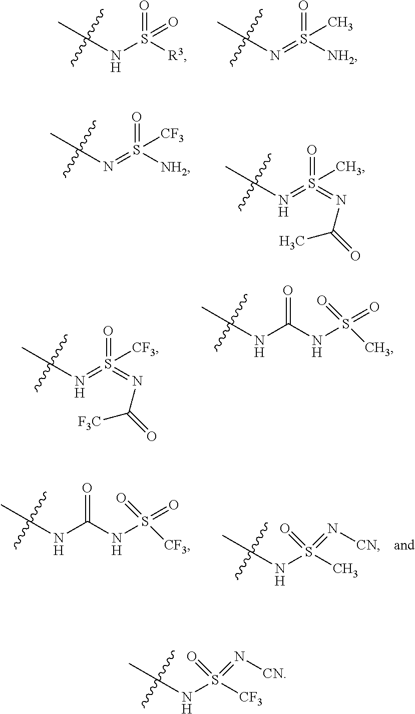

In certain embodiments, in the compound of Formula II, R^{2A} is selected from

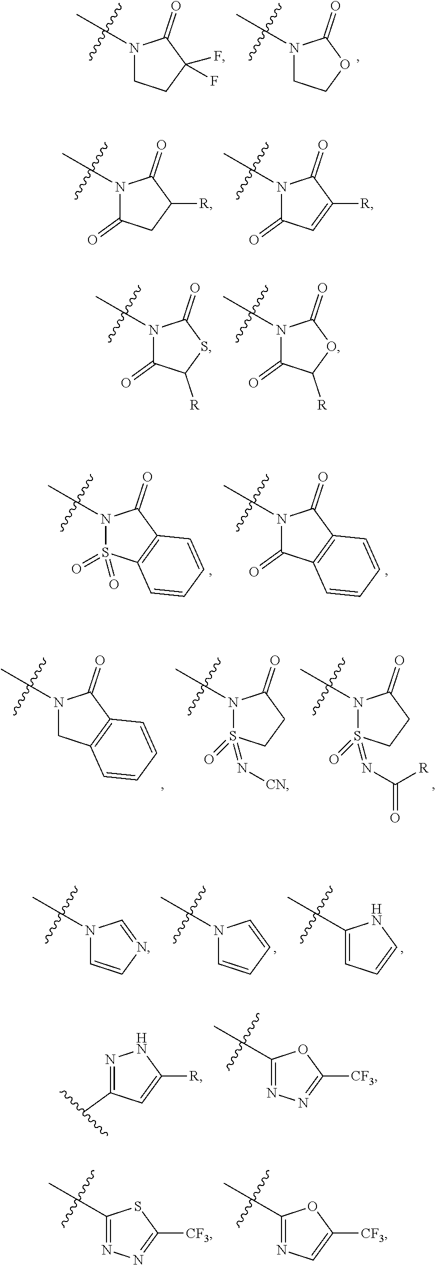

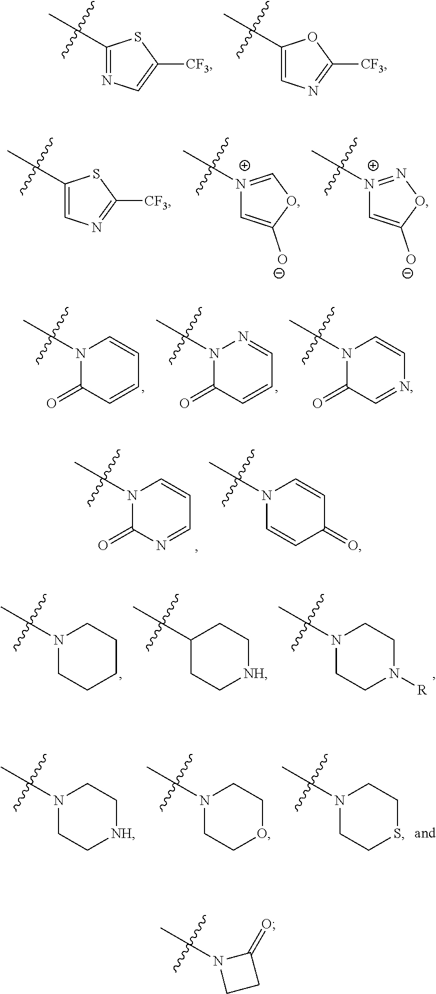

wherein R is an optional substituent as defined herein.

In certain embodiments, in the compound of Formula II, R^{2A} is selected from

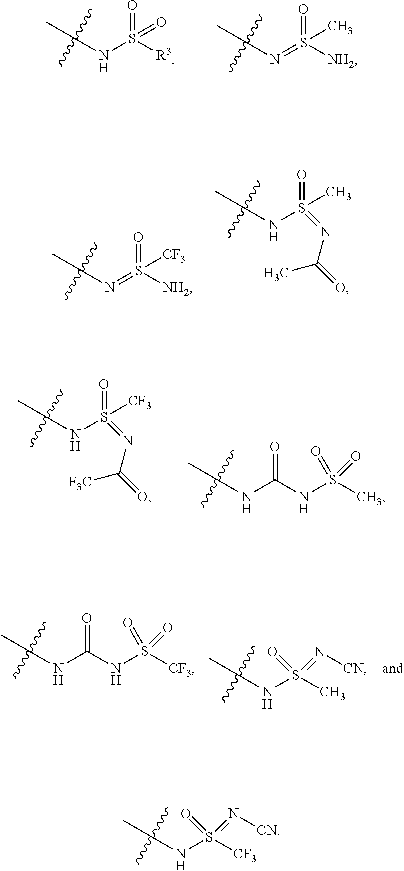

In certain embodiments, in the compound of Formula II, R² is selected from

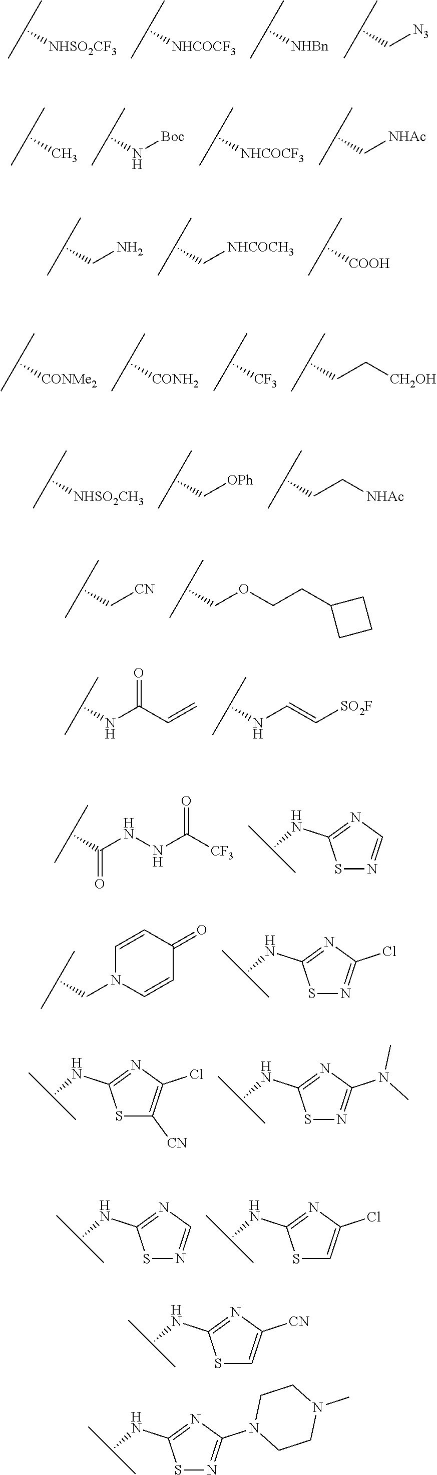

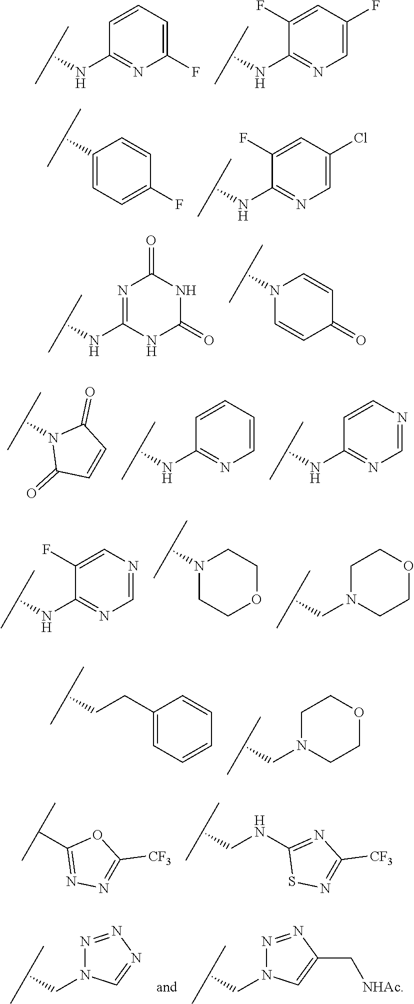

In certain embodiments, in the compound of Formula II, R² is selected from

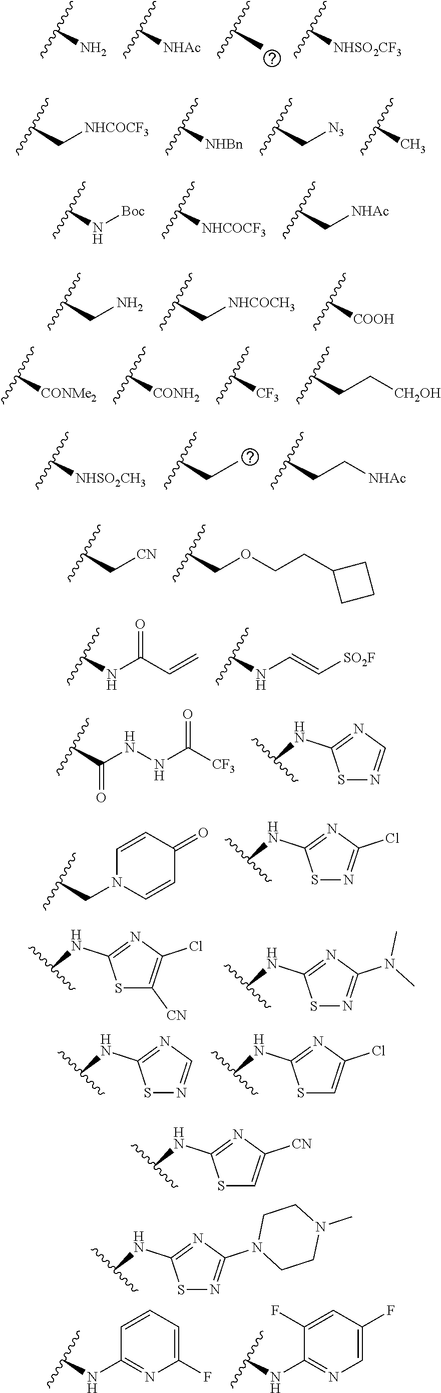

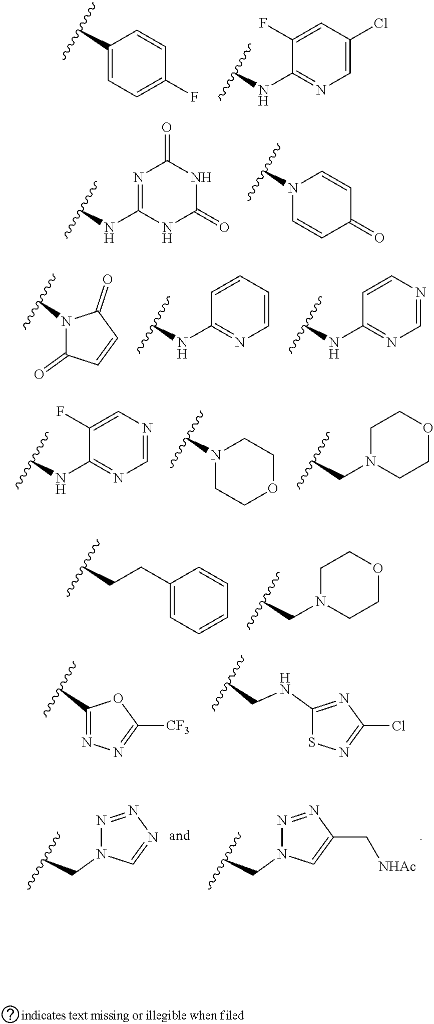

In certain embodiments, in the compound of Formula II, R² is selected from

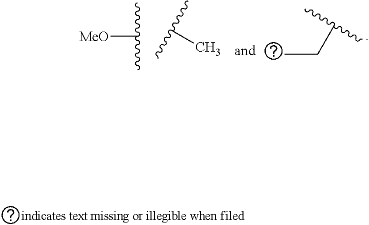

In certain embodiments, in the compound of Formula II, R² is selected from

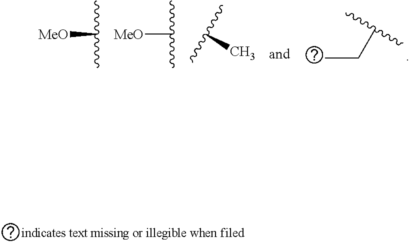

In certain embodiments, in the compound of Formula II, R² is selected from

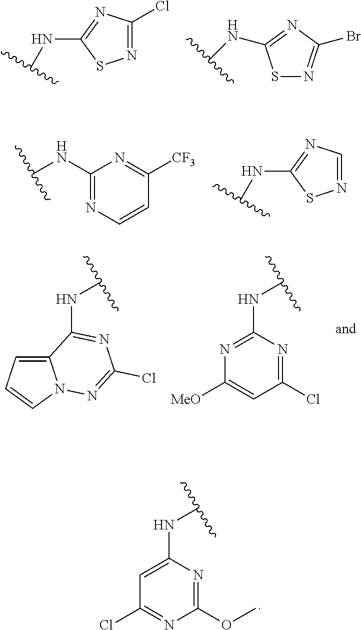

In certain embodiments, in the compound of Formula II, R² is selected from

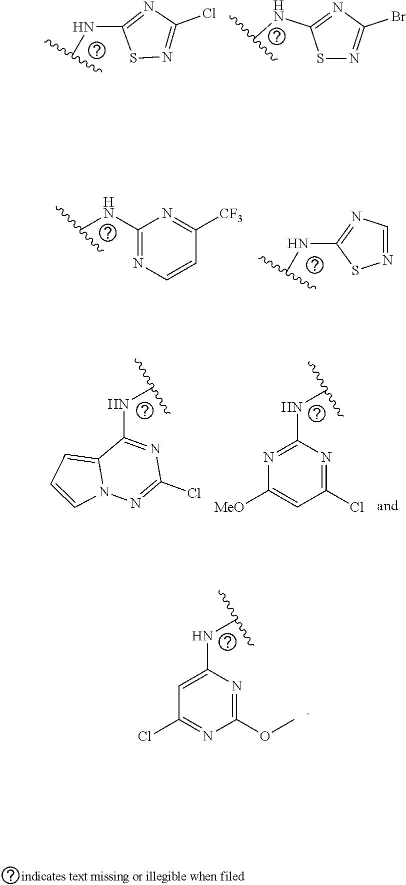

In certain embodiments, in the compound of Formula II, R² is selected from

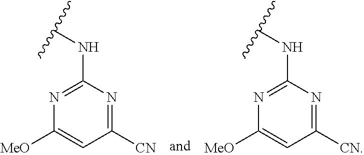 [SMILES: *Nc1nc(C#N)cc(OC)n1 ; *Nc1nc(C#N)cc(OC)n1]

In certain embodiments, in the compound of Formula II, R² is selected from

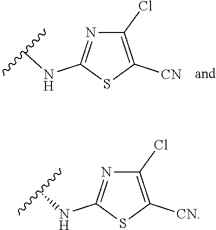

In certain embodiments, in the compound of Formula II, R² is selected from

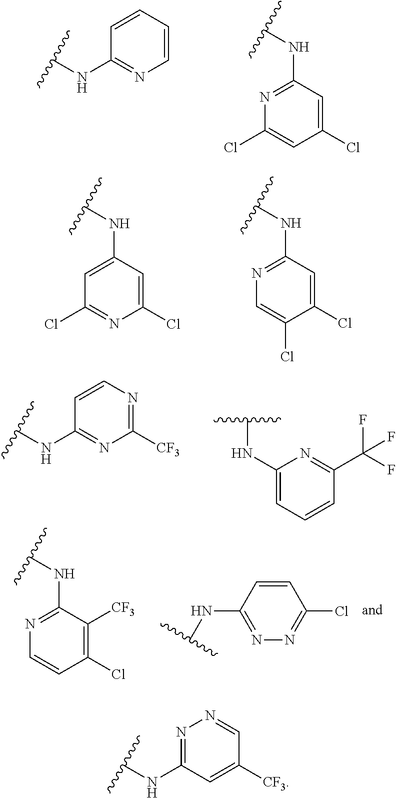

In certain embodiments, in the compound of Formula II, R² is selected from

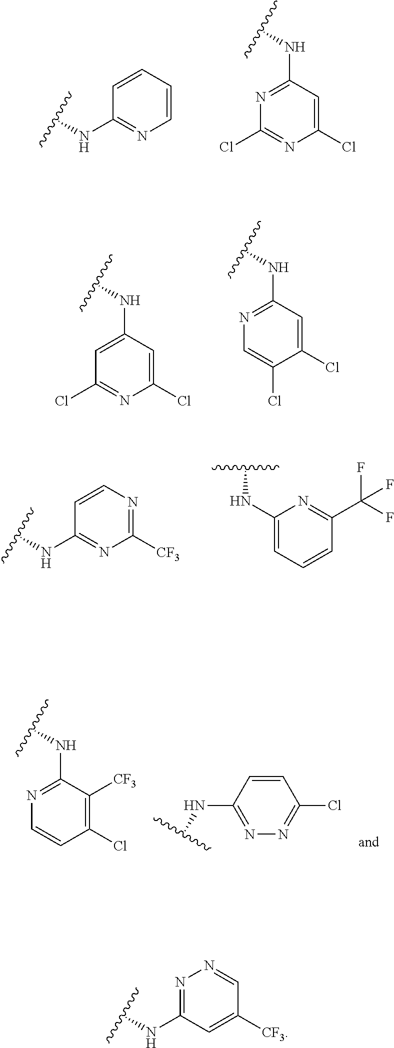

In certain embodiments, in the compound of Formula II, R² is selected from

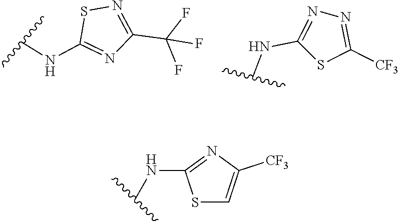 [SMILES: *Nc1nc(C(F)(F)F)ns1 ; *Nc1nnc(C(F)(F)F)s1 ; *Nc1nc(C(F)(F)F)cs1]

In certain embodiments, in the compound of Formula II, R² is selected from

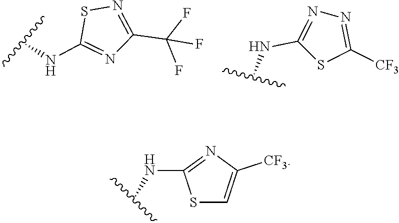

In certain embodiments, in the compound of Formula II, R² is selected from

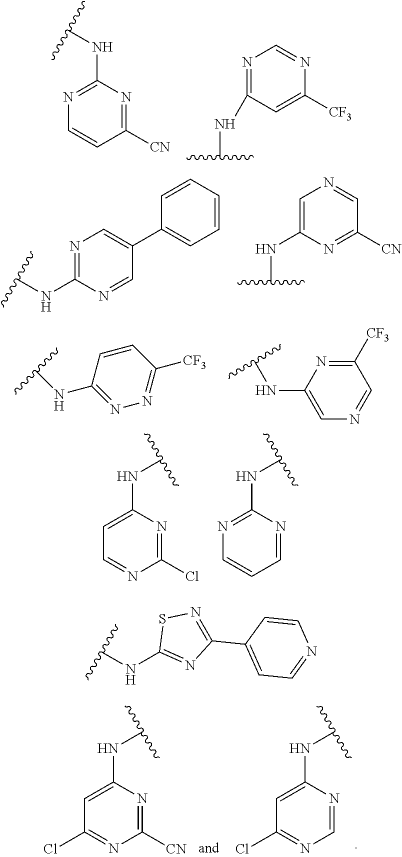

In certain embodiments, in the compound of Formula II, R² is selected from

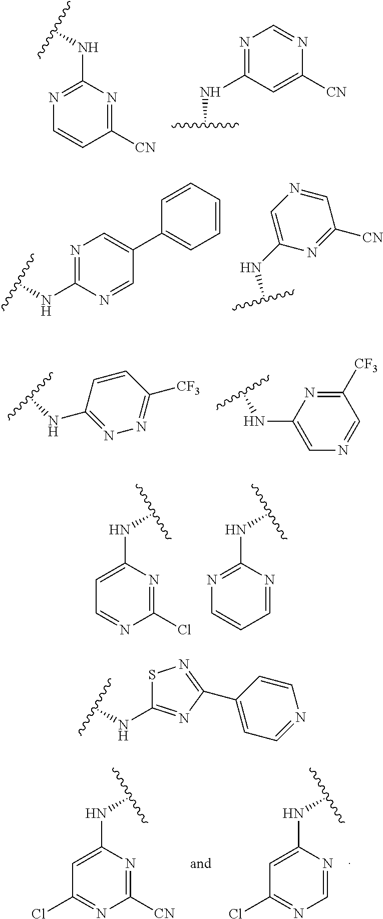

In certain embodiments, in the compound of Formula II, R² is selected from

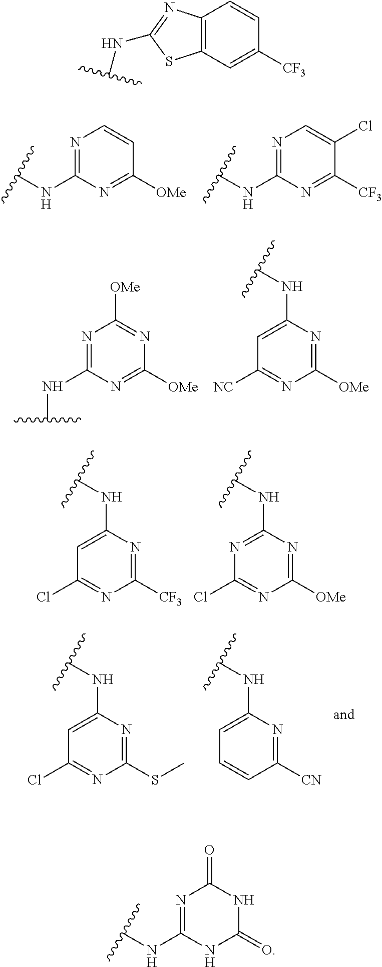

In certain embodiments, in the compound of Formula II, R² is selected from

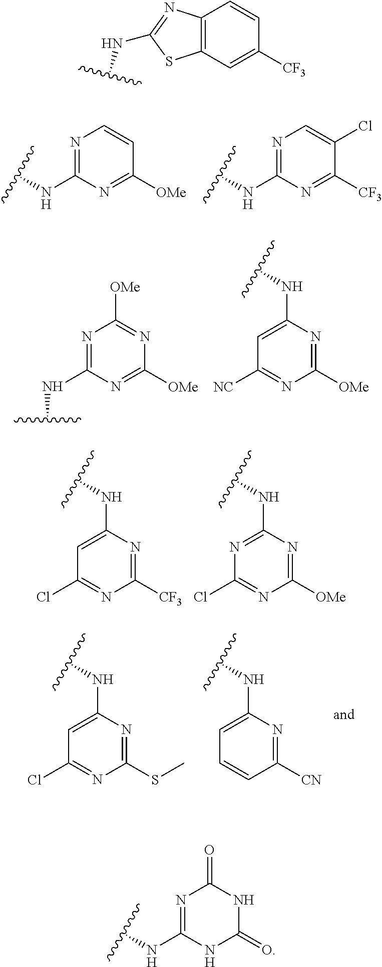

In certain embodiments, in the compound of Formula II, R² is selected from

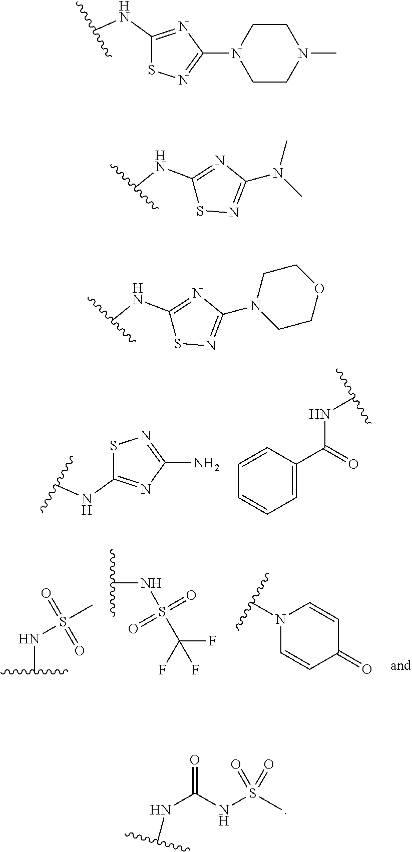

In certain embodiments, in the compound of Formula II, R² is selected from

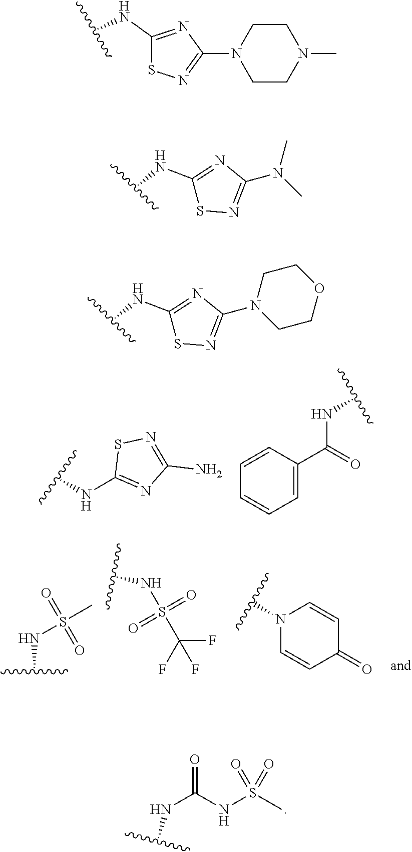

In certain embodiments, in the compound of Formula II, R² is selected from

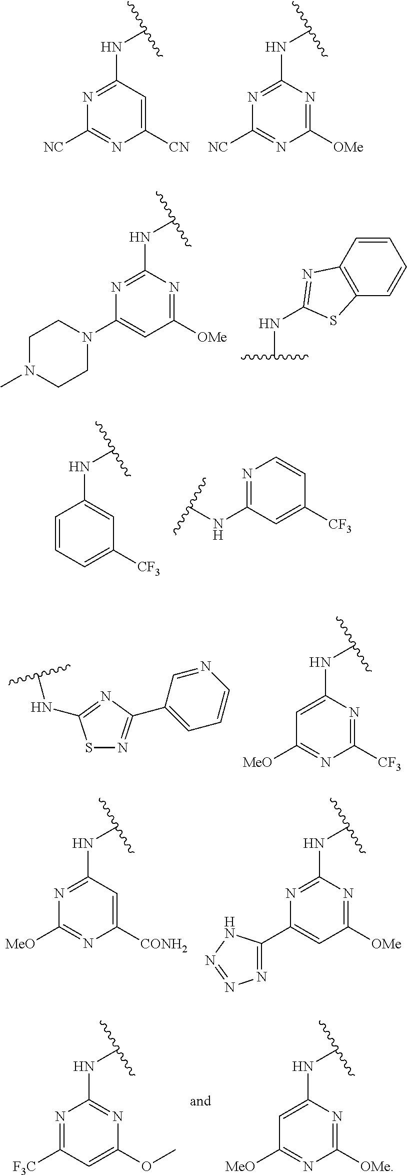

In certain embodiments, in the compound of Formula II, R² is selected from

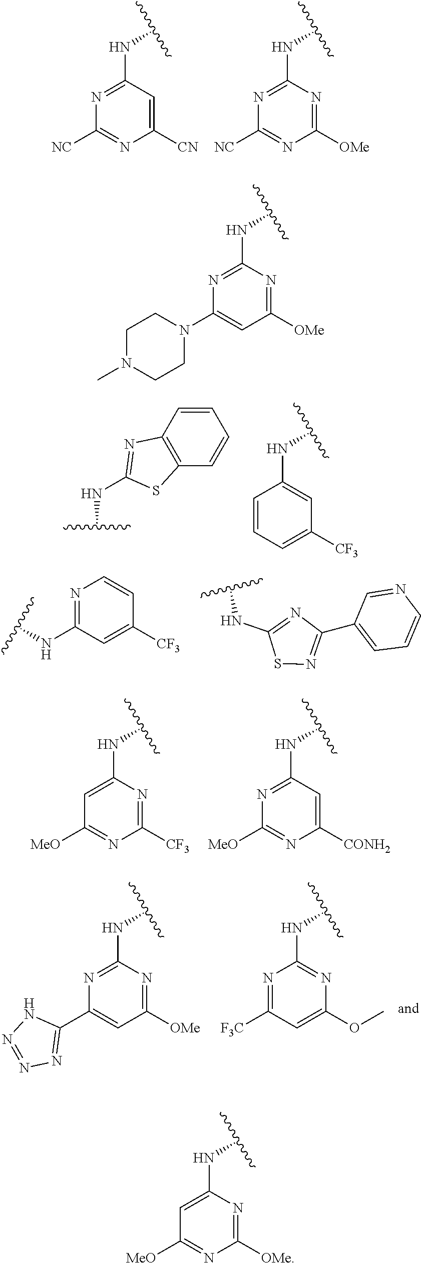

In certain embodiments, in the compound of Formula II, R² is selected from

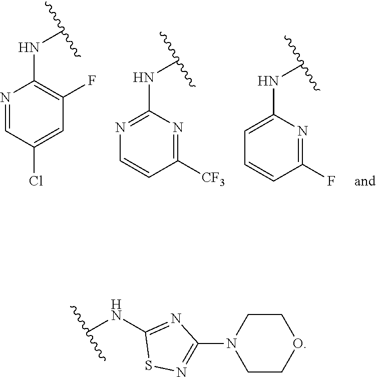

In certain embodiments, in the compound of Formula II, R² is selected from

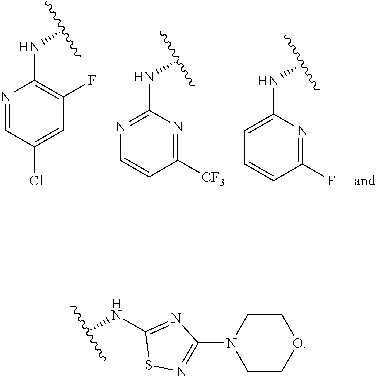

In certain embodiments, in the compound of Formula II, R² is selected from

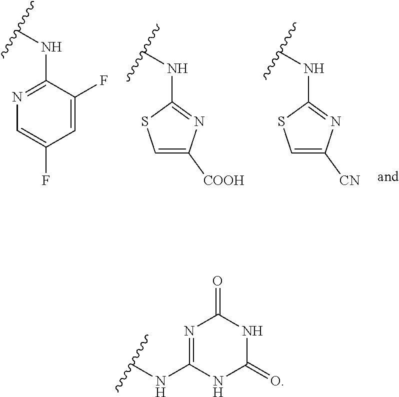 [SMILES: *Nc1ncc(F)cc1F ; *Nc1nc(C(=O)O)cs1 ; *Nc1nc(C#N)cs1 ; *Nc1nc(=O)[nH]c(=O)[nH]1]

In certain embodiments, in the compound of Formula II, R² is selected from

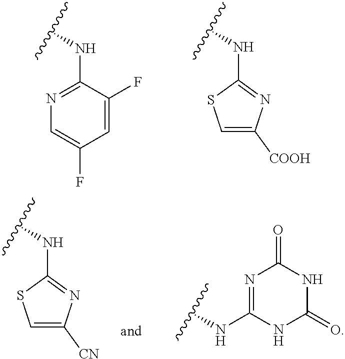

In certain embodiments, in the compound of Formula II, R² is selected from

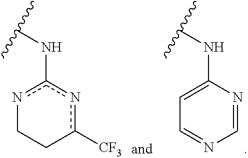

In certain embodiments, in the compound of Formula II, R² is selected from

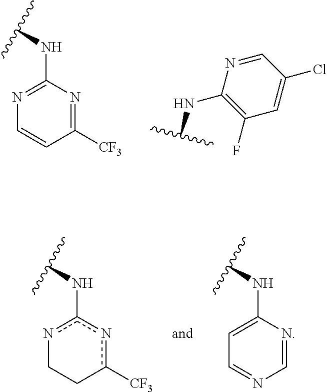

In certain embodiments, in the compound of Formula II, R² is selected from

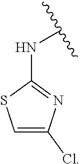 [SMILES: *Nc1nc(Cl)cs1]

In certain embodiments, in the compound of Formula II, R² is selected from

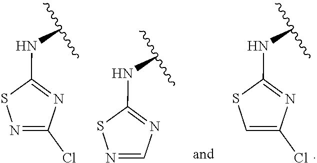

In certain embodiments, in the compound of Formula II, R² is selected from

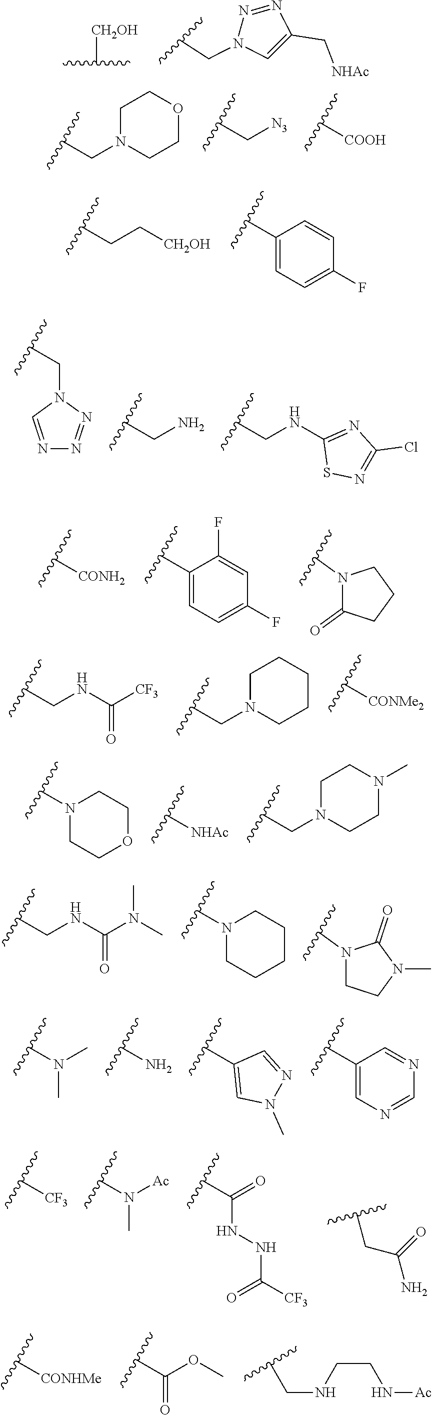

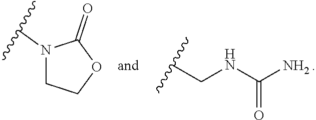 [SMILES: *N1CCOC1=O ; *CNC(N)=O]

In certain embodiments, in the compound of Formula II, R² or R^{2A} is selected from

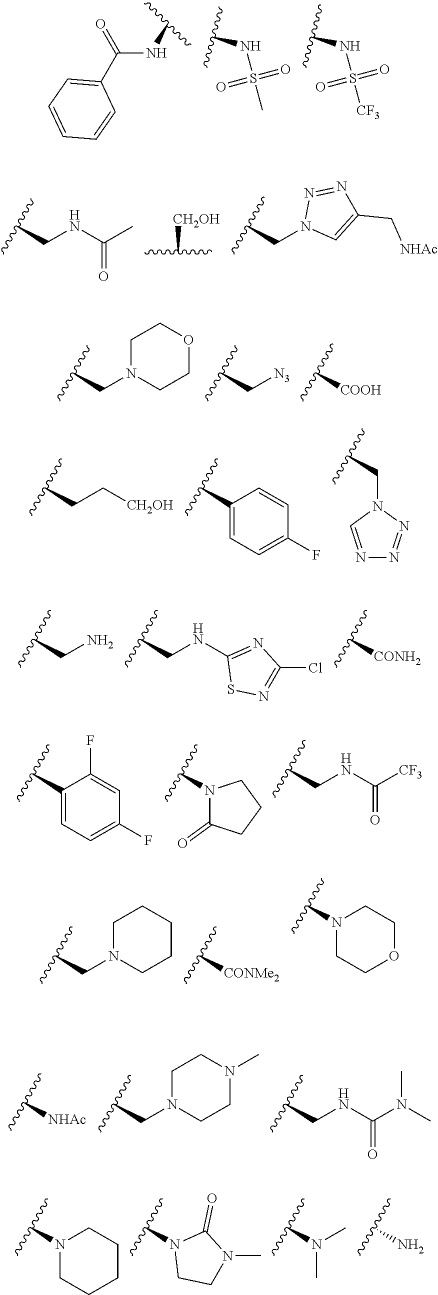

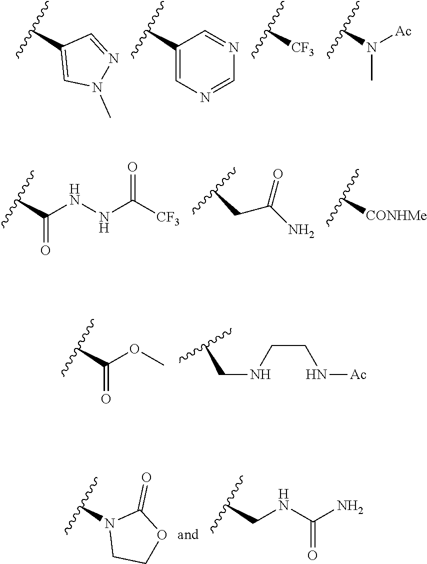

In certain embodiments, in the compound of Formula II, R² is selected from

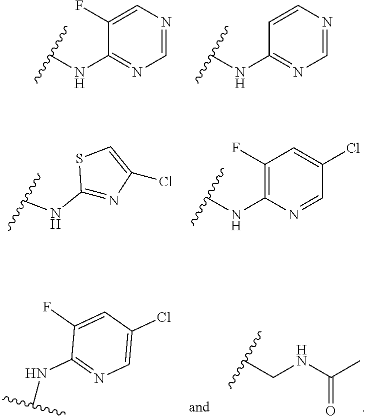

In certain embodiments, in the compound of Formula II, R² is selected from

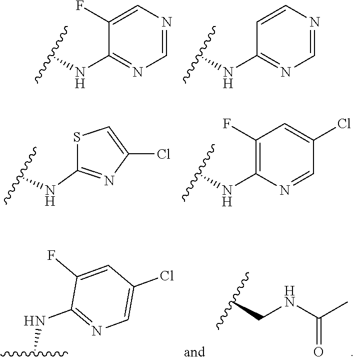

In certain embodiments, in the compound of Formula II, R² is selected from

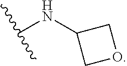 [SMILES: *NC1COC1]

In certain embodiments, in the compound of Formula II, R² is selected from

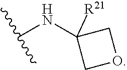

In certain embodiments, in the compound of Formula II, R² is selected from

In certain embodiments, in the compound of Formula II, R² is a spirocyclic heterocycle, for example, and without limitation,

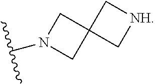 [SMILES: *N1CC2(CNC2)C1]

In certain embodiments, in the compound of Formula II, R² is a silicon containing heterocycle, for example, and without limitation,

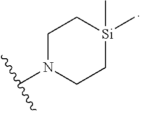 [SMILES: *N1CC[Si](C)(C)CC1]

In certain embodiments, in the compound of Formula II, R² is substituted with SFs, for example, and without limitation,

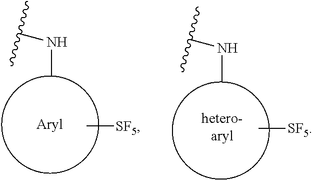

In certain embodiments, in the compound of Formula II, R² is substituted with a sulfoxime, for example, and without limitation,

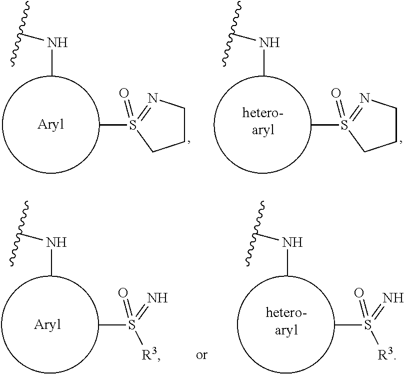

In certain embodiments, in the compound of Formula II, R¹⁰ is selected from bicyclic heterocycle.

In certain embodiments, in the compound of Formula II, R¹⁰ is selected from spirocyclic heterocycle.

In certain embodiments, in the compound of Formula II, R¹⁰ is selected from —NR-heterocycle.

In certain embodiments, in the compound of Formula II, R¹⁰ is selected from

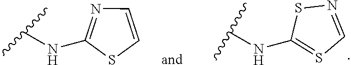

In certain embodiments, in the compound of Formula II, R¹⁰ is selected from

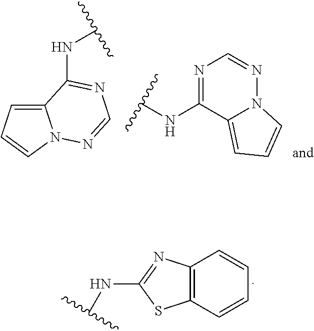

In certain embodiments, in the compound of Formula I, R¹⁰ is selected from

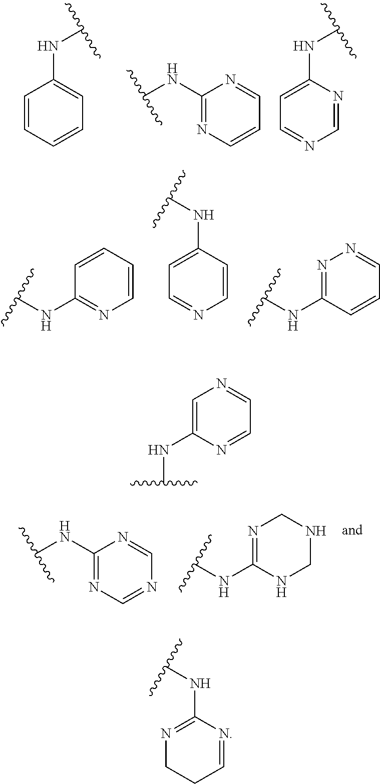

In certain embodiments, in the compound of Formula II, R¹⁰ is selected from

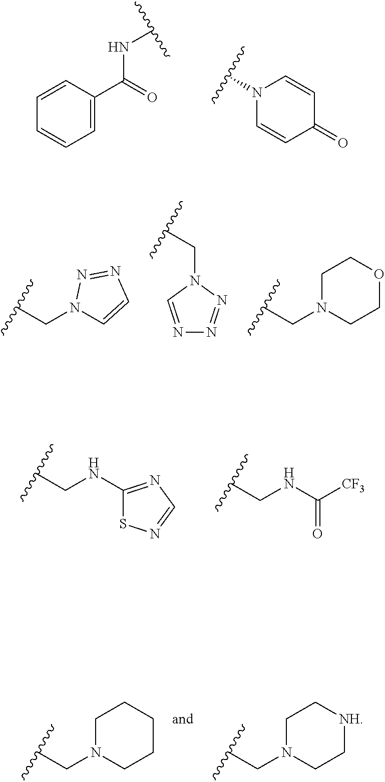

In certain embodiments, in the compound of Formula II, Cycle is selected from

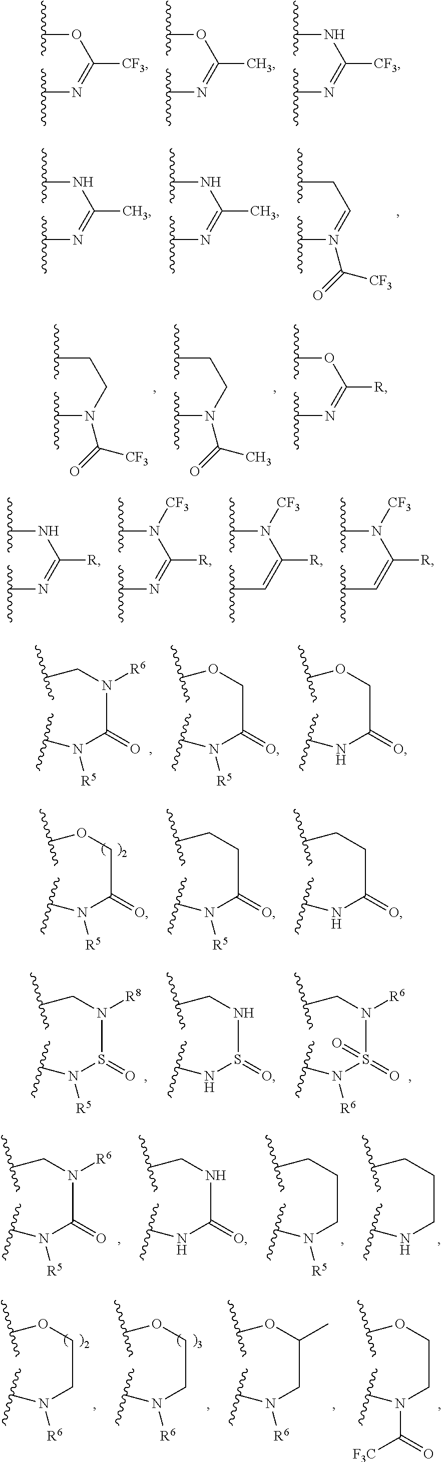

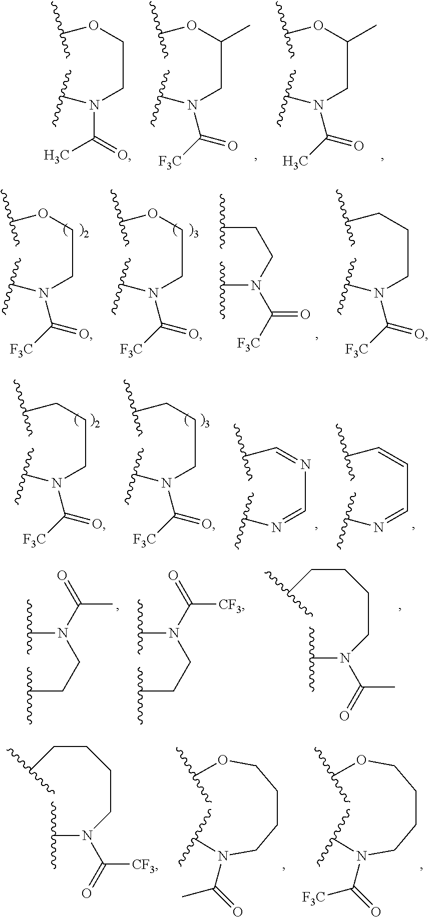

In certain embodiments, in the compound of Formula II, R³⁰ is

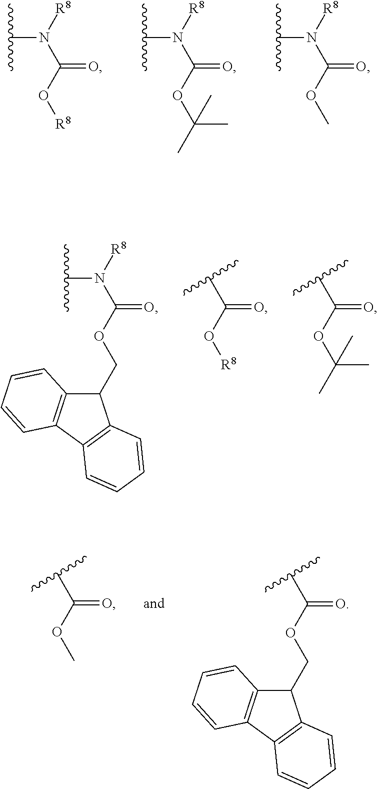

In certain embodiments, in the compound of Formula II, R²⁰⁰ is

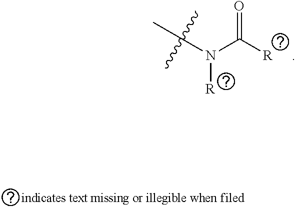

In certain embodiments, in the compound of Formula II, R²⁰⁰ is

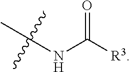

In certain embodiments, in the compound of Formula II, R²⁰⁰ is

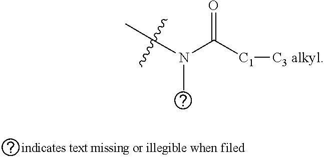

In certain embodiments, in the compound of Formula II, R²⁰⁰ is

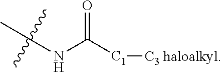

In certain embodiments, in the compound of Formula II, R²⁰⁰ is

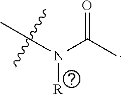

In certain embodiments, in the compound of Formula II, R²⁰⁰ is

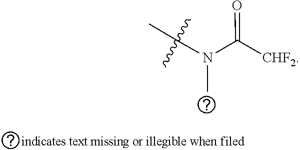 [SMILES: *N(C)C(=O)C(F)F]

In certain embodiments, in the compound of Formula II, R²⁰⁰ is

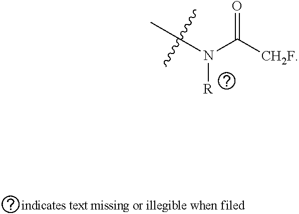

In certain embodiments, in the compound of Formula II, R²⁰⁰ is

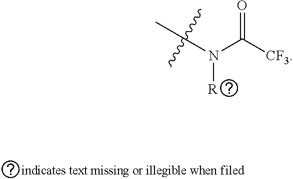

In certain embodiments, in the compound of Formula II, R²⁰⁰ is

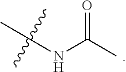 [SMILES: *NC(C)=O]

In certain embodiments, in the compound of Formula II, R²⁰⁰ is

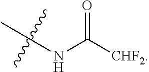 [SMILES: *NC(=O)C(F)F]

In certain embodiments, in the compound of Formula II, R²⁰⁰ is

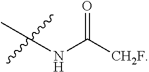

In certain embodiments, in the compound of Formula II, R²⁰⁰ is

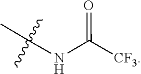 [SMILES: *NC(=O)C(F)(F)F]

### Linkers

In non-limiting embodiments, in the compound of Formula II, Linker^{A} and Linker^{B} are independently selected from:

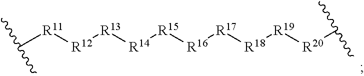

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂-, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, and, heterocycle, heteroaryl, —CH₂CH₂-[O—(CH₂)₂]ₙ—O—, CH₂CH₂-[O—(CH₂)₂]ₙ—NR⁶—, —CH₂CH₂-[O—(CH₂)₂]ₙ-, -[—(CH₂)₂—O—]ₙ-, -[O—(CH₂)₂]ₙ-, -[O—CH(CH₃)C(O)]ₙ-, -[C(O)—CH(CH₃)—O]ₙ-,
[O—CH₂C(O)]ₙ-, -[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, and, heteroaryl, and heterocycle; and the remaining variables are as defined herein.

In certain embodiments, in the compound of Formula II, Linker^{A} is bond and Linker^{B} is

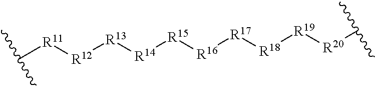

In certain embodiments, in the compound of Formula II, Linker^{B} is bond and Linker^{A} is

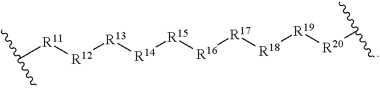

In certain embodiments, in the compound of Formula II, a divalent residue of an amino acid is selected from

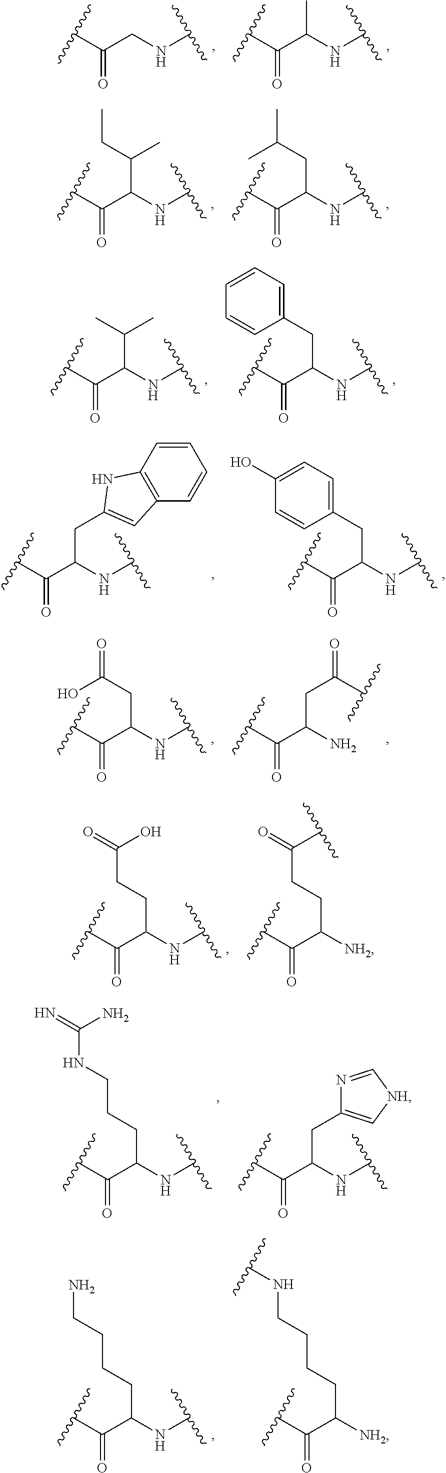

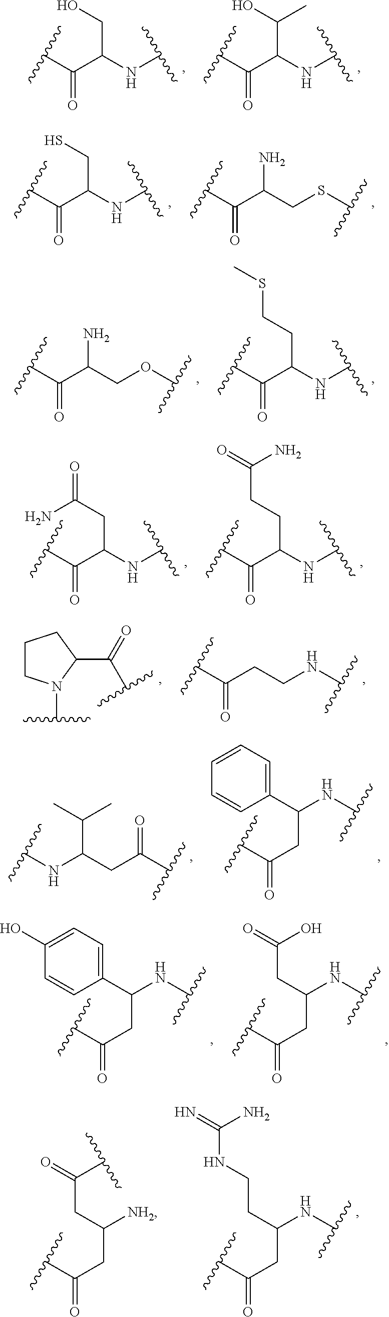

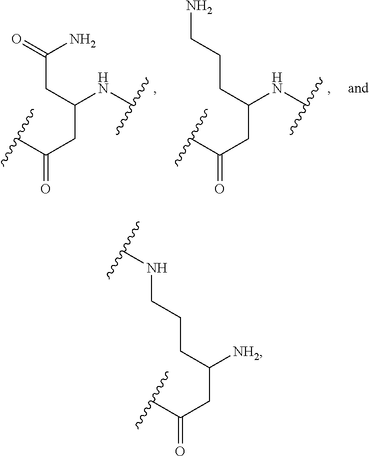 [SMILES: *NC(CC(*)=O)CC(N)=O ; *NC(CCCN)CC(*)=O ; *NCCCC(N)CC(*)=O]

wherein the amino acid can be oriented in either direction and wherein the amino acid can be in the L- or D-form or a mixture thereof.

In certain embodiments, in the compound of Formula II, a divalent residue of a dicarboxylic acid is generated from a nucleophilic addition reaction:

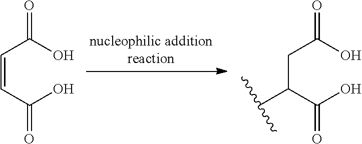 [SMILES: O=C(O)/C=C\C(=O)O ; *C(CC(=O)O)C(=O)O]

Non-limiting embodiments of a divalent residue of a dicarboxylic acid generated from a nucleophilic addition reaction include:

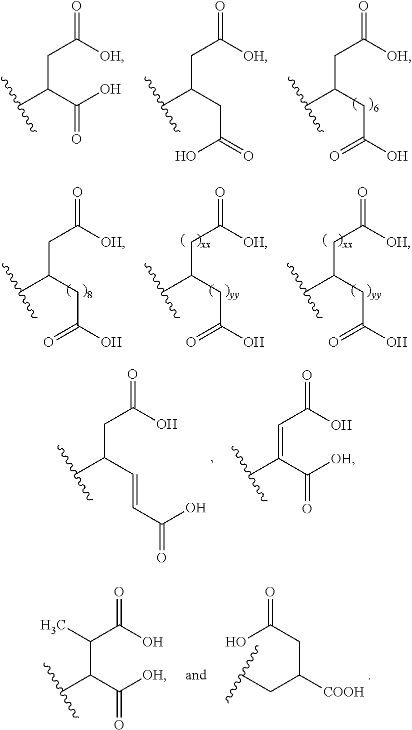

In certain embodiments, in the compound of Formula II, a divalent residue of a dicarboxylic acid is generated from a condensation reaction:

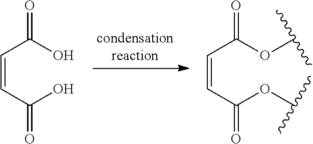 [SMILES: O=C(O)/C=C\C(=O)O ; *OC(=O)/C=C\C(=O)O*]

Non-limiting embodiments of a divalent residue of a dicarboxylic acid generated from a condensation include:

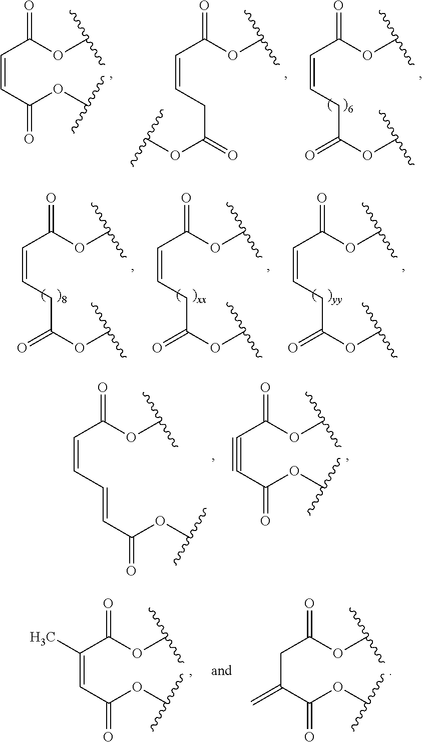

Non-limiting embodiments of a divalent residue of a saturated dicarboxylic acid include:

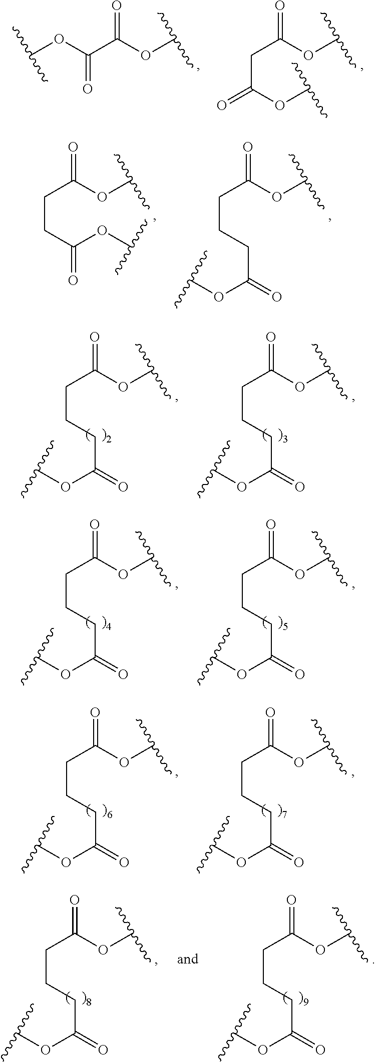

Non-limiting embodiments of a divalent residue of a saturated dicarboxylic acid include:

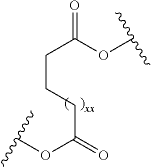

Non-limiting embodiments of a divalent residue of a saturated monocarboxylic acid is selected from butyric acid (—OC(O)(CH₂)₂CH₂-), caproic acid (—OC(O)(CH₂)₄CH₂-), caprylic acid (—OC(O)(CH₂)₅CH₂-), capric acid (—OC(O)(CH₂)₈CH₂-), lauric acid (—OC(O)(CH₂)₁₀CH₂-), myristic acid (—OC(O)(CH₂)₁₂CH₂-), pentadecanoic acid (—OC(O)(CH₂)₁₃CH₂-), palmitic acid (—OC(O)(CH₂)₁₄CH₂-), stearic acid (—OC(O)(CH₂)₁₆CH₂-), behenic acid (—OC(O)(CH₂)₂₀CH₂-), and lignoceric acid (—OC(O)(CH₂)₂₂CH₂-);

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

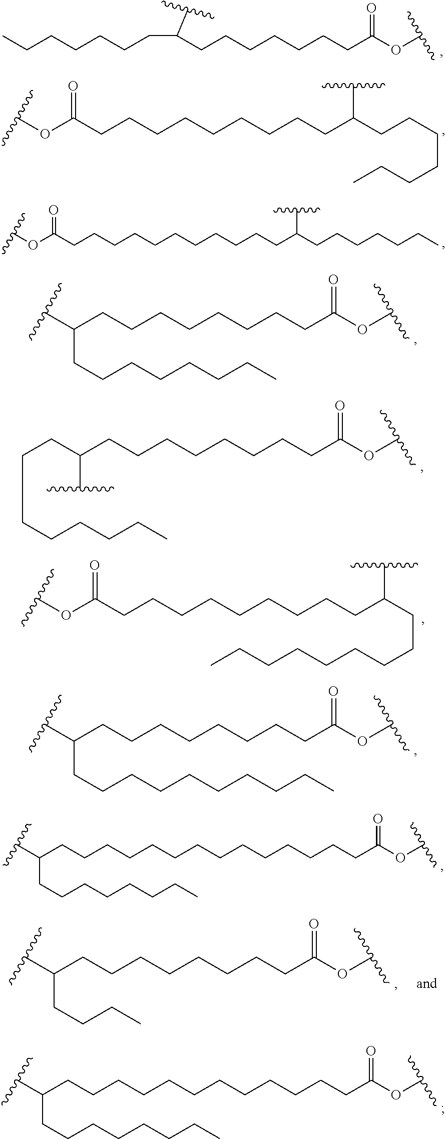

Non-limiting embodiments of a divalent residue of a fatty acid is selected from linoleic acid (—C(O)(CH₂)₇(CH)₂CH₂(CH)₂(CH₂)₄CH₂-), docosahexaenoic acid
(—C(O)(CH₂)₂(CHCHCH₂)₆CH₂-), eicosapentaenoic acid (—C(O)(CH₂)₃(CHCHCH₂)₅CH₂-), alpha-linolenic acid (—C(O)(CH₂)₇(CHCHCH₂)₃CH₂-) stearidonic acid
(—C(O)(CH₂)₄(CHCHCH₂)₄CH₂-), y-linolenic acid (—C(O)(CH₂)₄(CHCHCH₂)₃(CH₂)₃CH₂-), arachidonic acid (—C(O)(CH₂)₃, (CHCHCH₂)₄(CH₂)₄CH₂—), docosatetraenoic acid
(—C(O)(CH₂)₅(CHCHCH₂)₄(CH₂)₄CH₂-), palmitoleic acid (—C(O)CH₂)₇CHCH(CH₂)₅CH₂—), vaccenic acid (—C(O)(CH₂)₉CHCH(CH₂)₅CH₂—), paullinic acid
(—C(O)(CH₂)HCHCH(CH₂)₅CH₂—), oleic acid (—C(O)(CH₂)₇CHCH(CH₂)₇CH₂—), elaidic acid
(—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₃CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments, in the compound of Formula II, Linker^{C} is selected from:

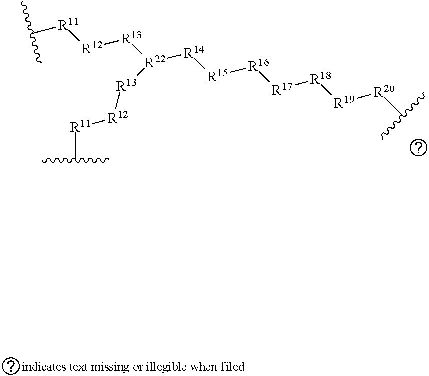

wherein:

R²² is independently at each occurrence selected from the group consisting of alkyl, —C(O)N—, —NC(O)—, —N—, —C(R²¹)—, —P(O)O—, —P(O)—, —P(O)(NR⁶R⁷)N-, alkenyl, haloalkyl, aryl, heterocycle, and heteroaryl, each of which is optionally substituted with 1, 2, 3, or 4 substituents independently selected from R²¹;
and the remaining variables are as defined herein.

In certain embodiments, in the compound of Formula II, Linker^{D} is selected from:

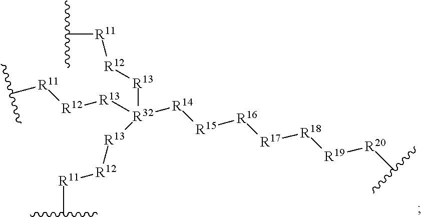

wherein:
R³² is independently at each occurrence selected from the group consisting of alkyl, N⁺X—, —C—, alkenyl, haloalkyl, aryl, heterocycle, and heteroaryl, each of which is optionally substituted with 1, 2, 3, or 4 substituents independently selected from R²¹;
X— is an anionic group, for example Br- or Cl⁻; and
all other variables are as defined herein.

In certain embodiments, in the compound of Formula II, Linker^{A} is selected from:

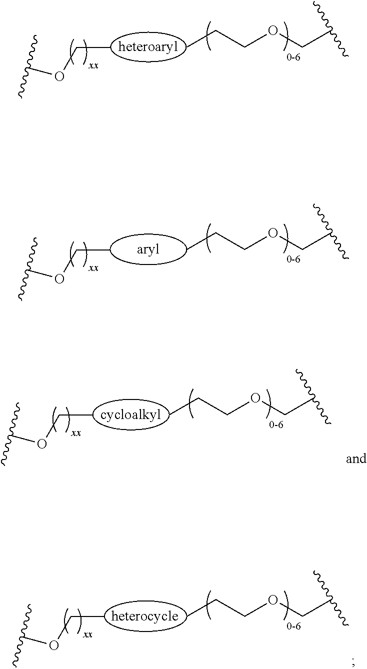

wherein each heteroaryl, heterocycle, cycloalkyl, and aryl can optionally be substituted with 1, 2, 3, or 4 of any combination of halogen, alkyl, haloalkyl, and, heteroaryl, heterocycle, or cycloalkyl, as allowed by valence.

In certain embodiments, in the compound of Formula II, Linker^{A} is selected from:

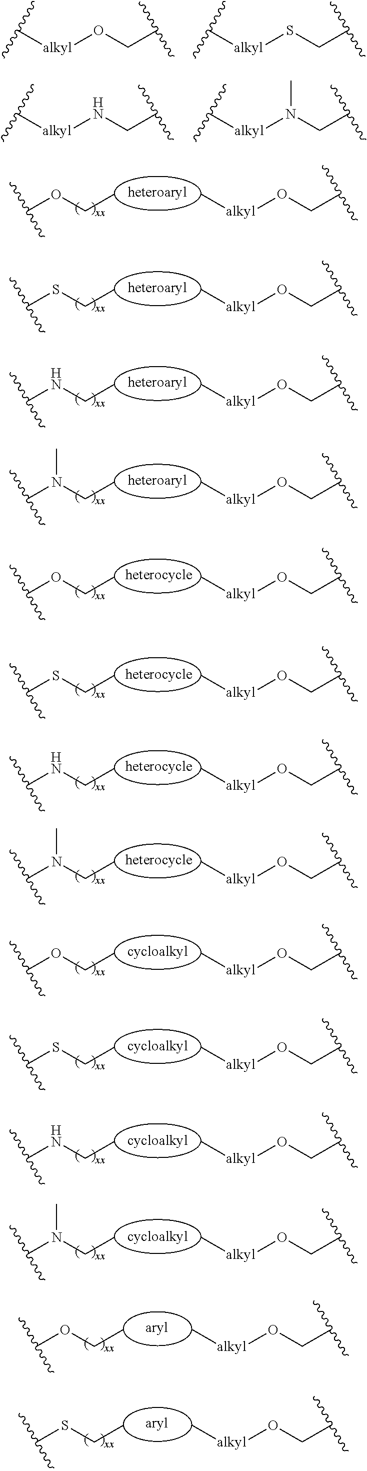

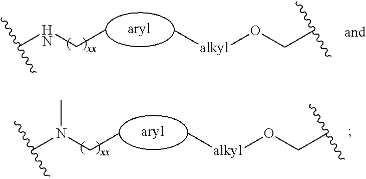

wherein each heteroaryl, heterocycle, cycloalkyl, and can optionally be substituted with 1, 2, 3, or 4 of any combination of halogen, alkyl, haloalkyl, aryl, heteroaryl, heterocycle, or cycloalkyl, as allowed by valence.

In certain embodiments, in the compound of Formula II, Linker^{B} is selected from:

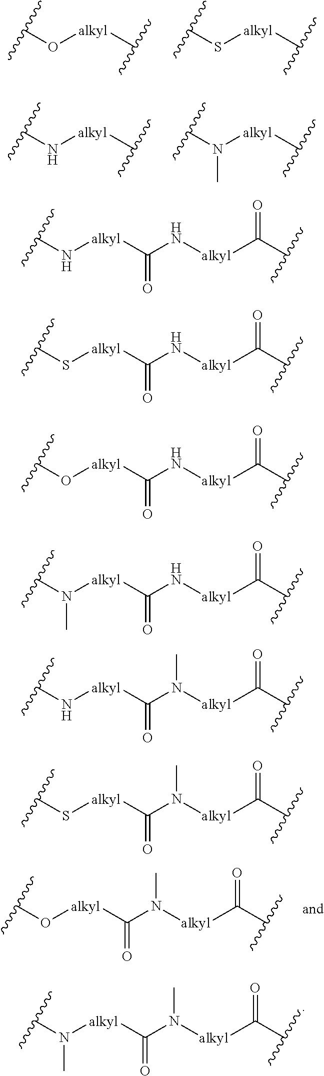

In certain embodiments, in the compound of Formula II, Linker^{B} is selected from:

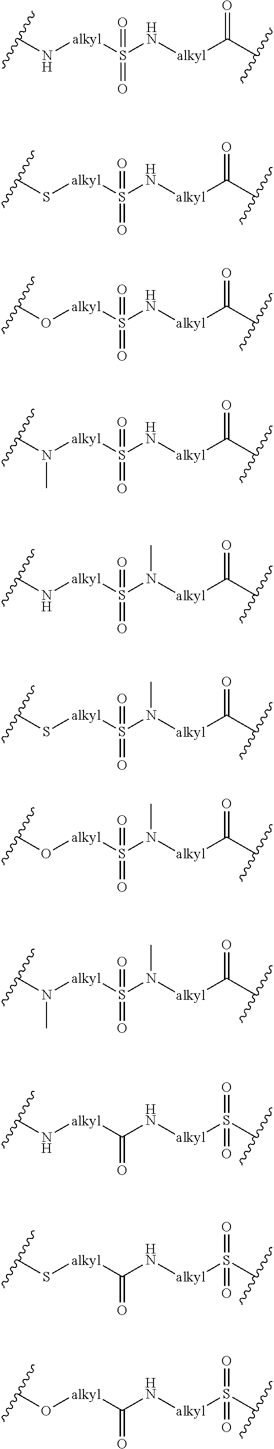

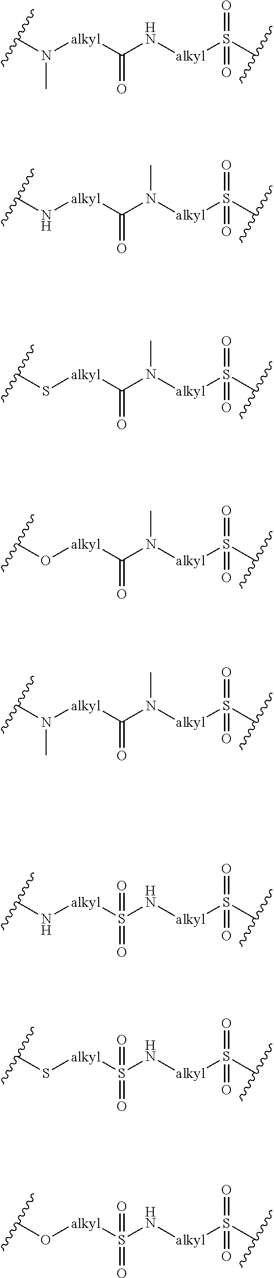

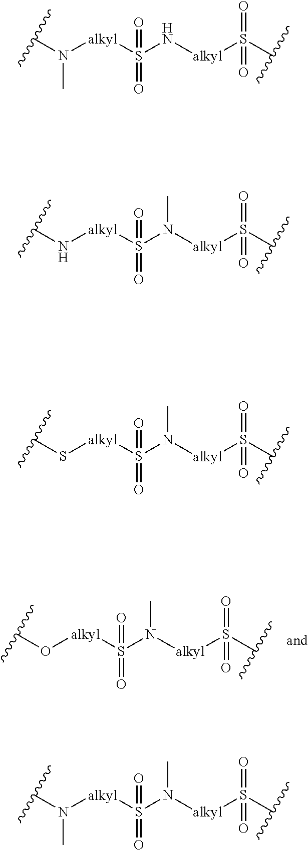

In certain embodiments, in the compound of Formula II, Linker^{B}, Linker^{C}, or Linker^{D} is selected from:

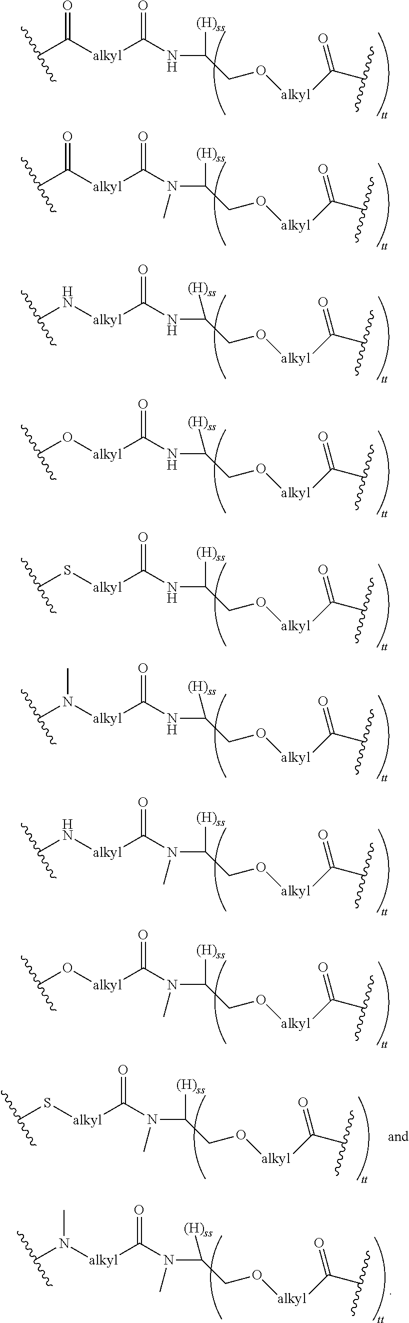

wherein tt is independently selected from 1, 2, or 3 and ss is 3 minus tt (3-tt).

In certain embodiments, in the compound of Formula II, Linker^{B}, Linker^{C}, or Linker^{D} is selected from:

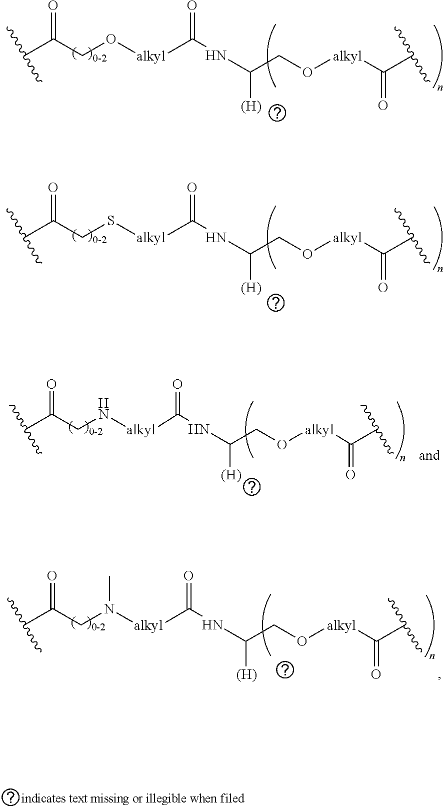

wherein tt and ss are as defined herein.

In certain embodiments, in the compound of Formula II, Linker^{B}, Linker^{C}, or Linker^{D} is selected from:

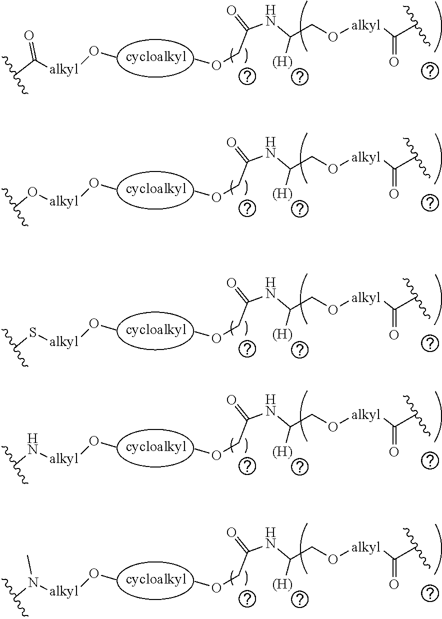

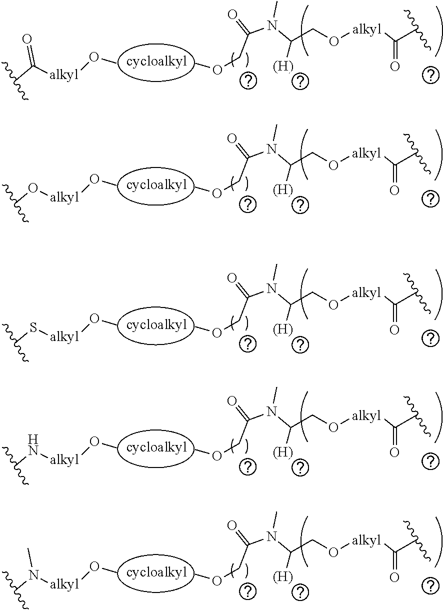

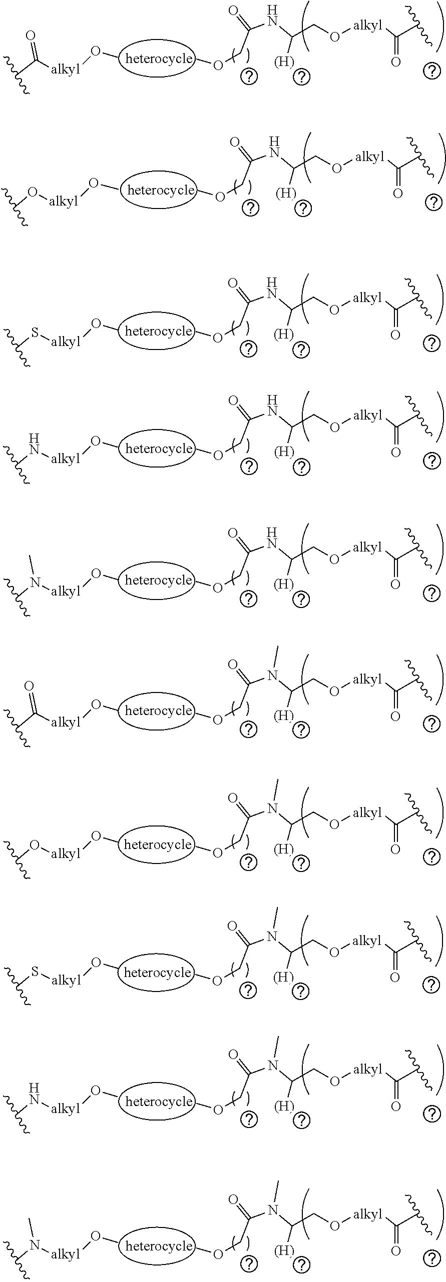

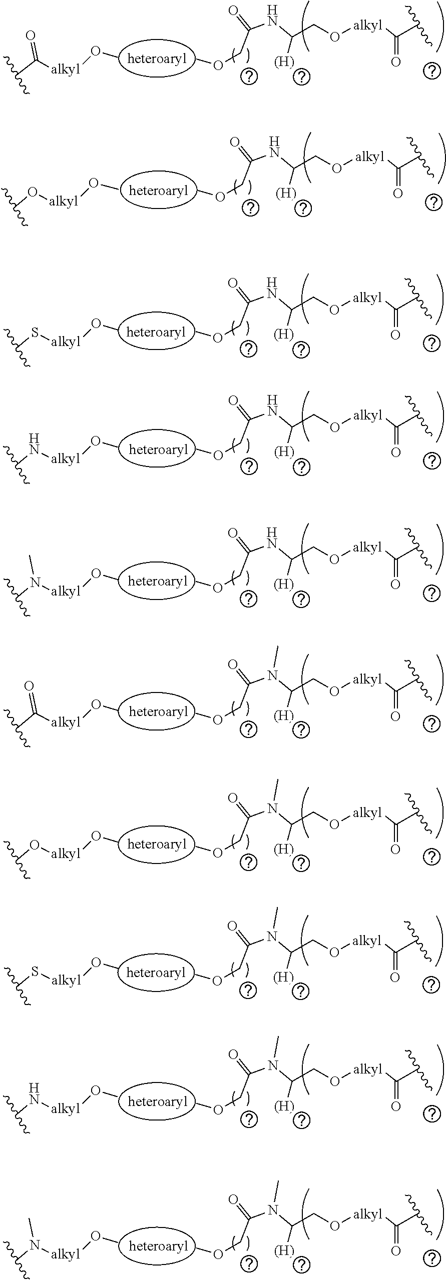

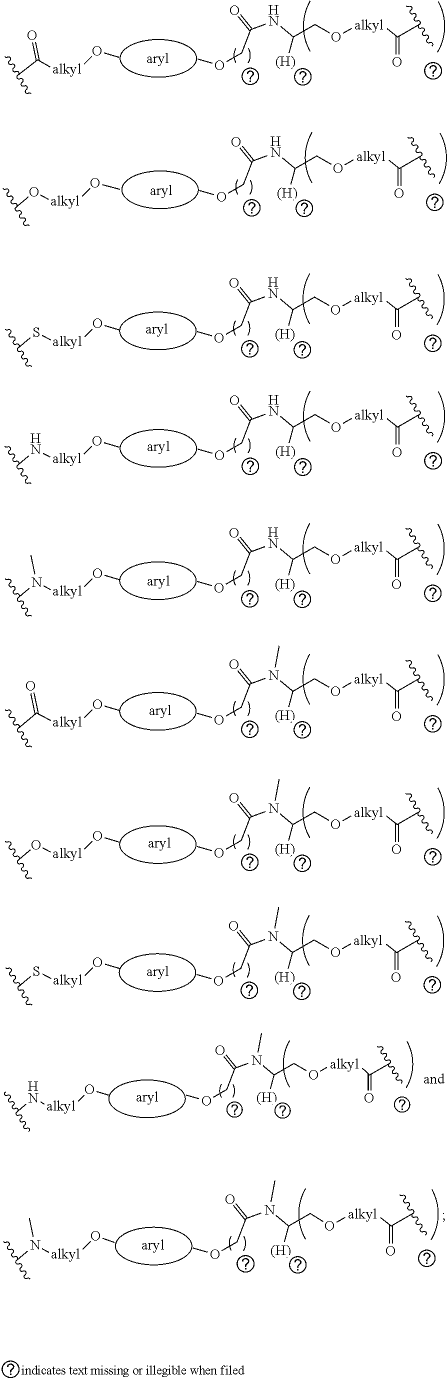

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{B}, Linker^{C}, or Linker^{D} is selected from:

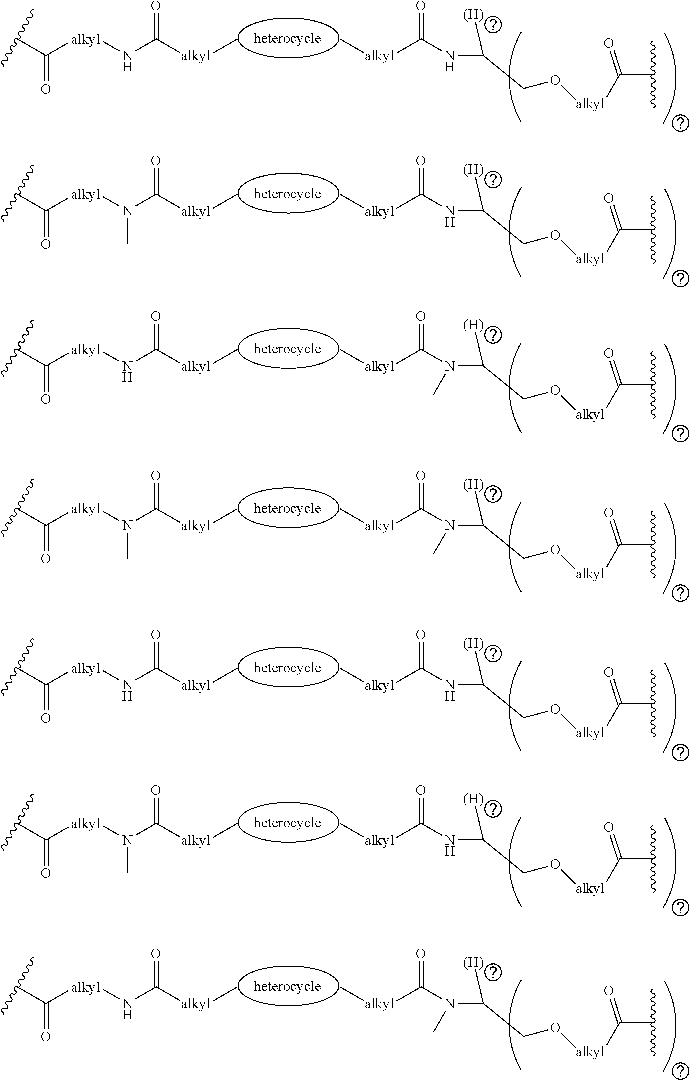

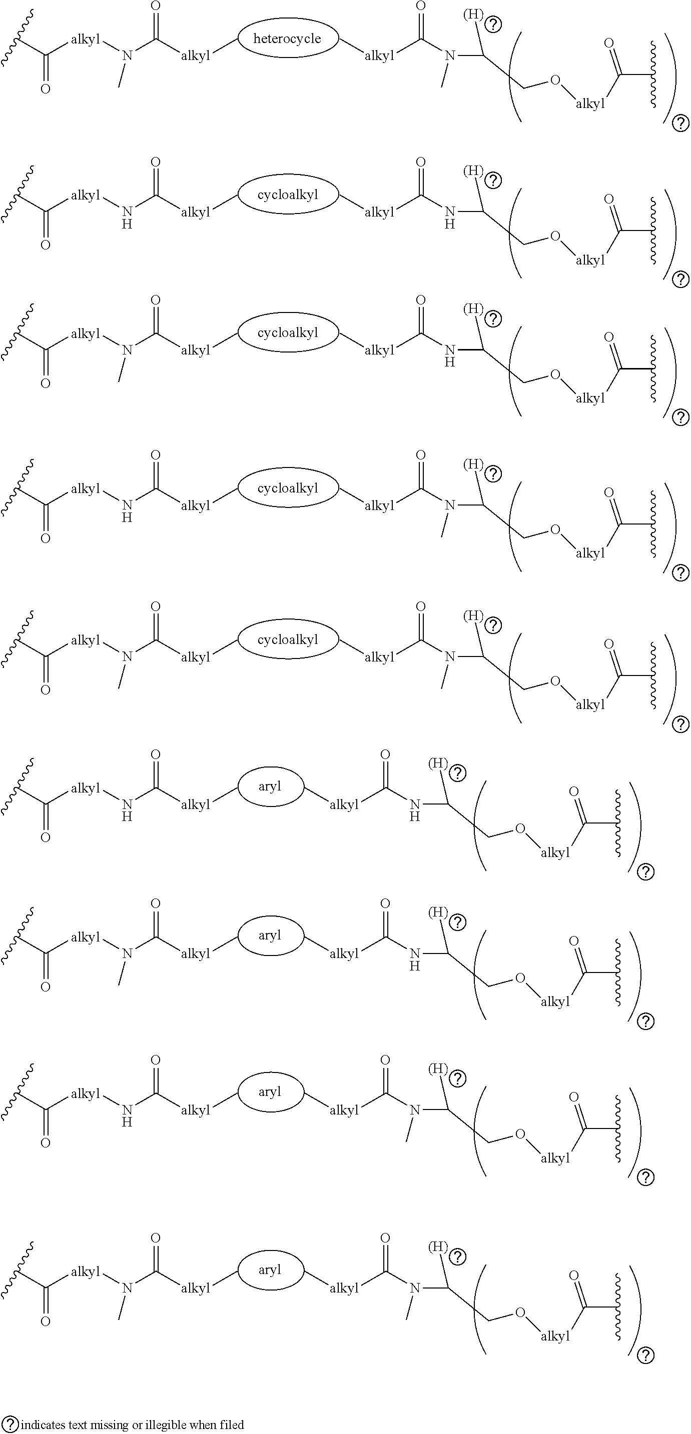

wherein each heteroaryl, heterocycle, cycloalkyl, and aryl can optionally be substituted with 1, 2 3, or 4 of any combination of halogen, alkyl, haloalkyl, and, heteroaryl, heterocycle, or cycloalkyl, as allowed by valence; and tt and ss are as defined herein.

In certain embodiments, in the compound of Formula II, Linker^{B}, Linker^{C}, or Linker^{D} is selected from:

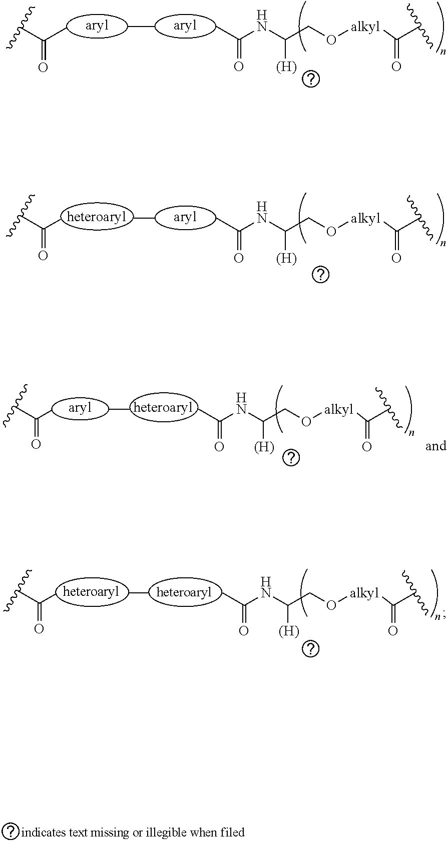

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{A} is selected from:

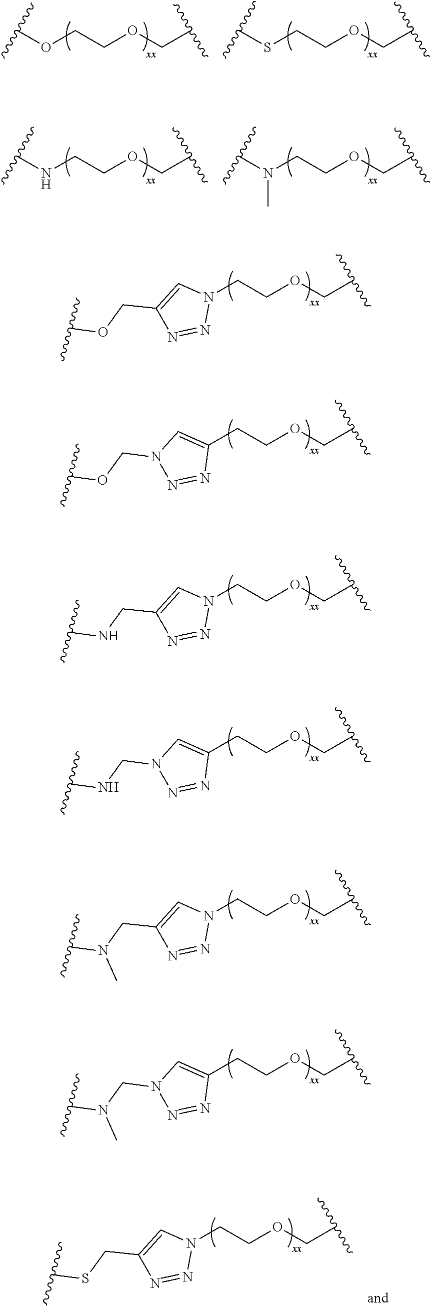

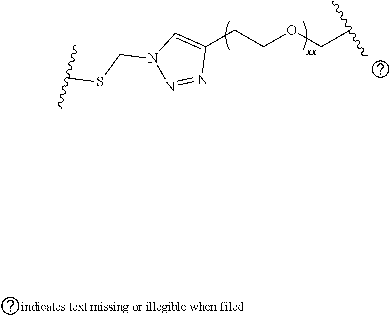

In certain embodiments, in the compound of Formula II Linker^{A} is selected from:

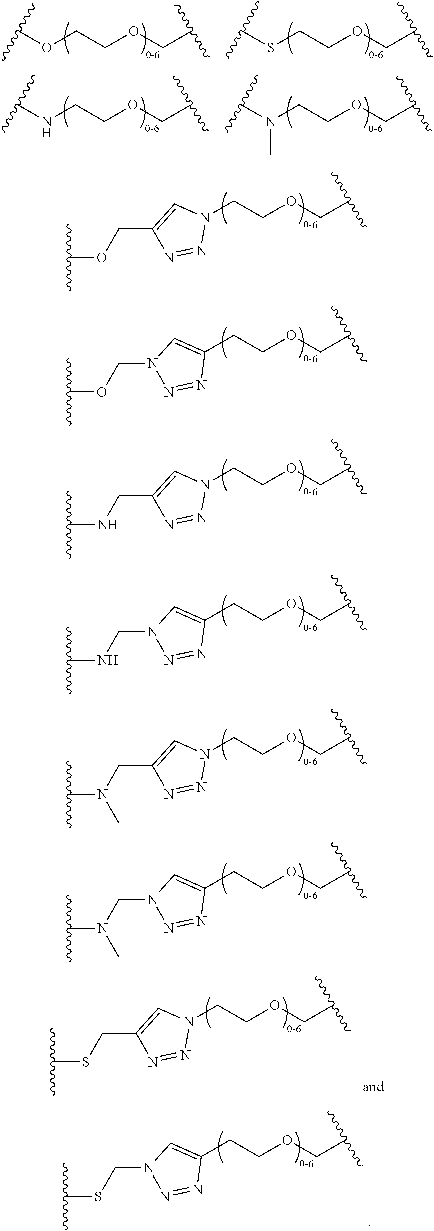

In certain embodiments, in the compound of Formula II, Linker^{A} is selected from:

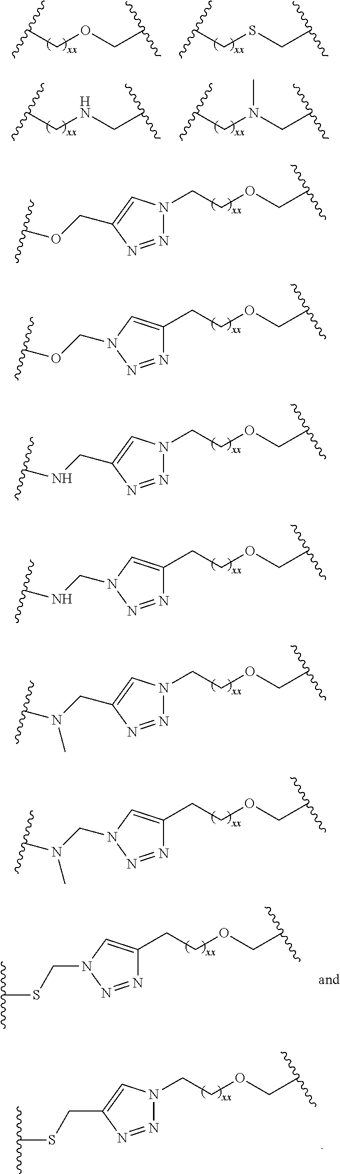

In certain embodiments, in the compound of Formula II, Linker^{A} is selected from:

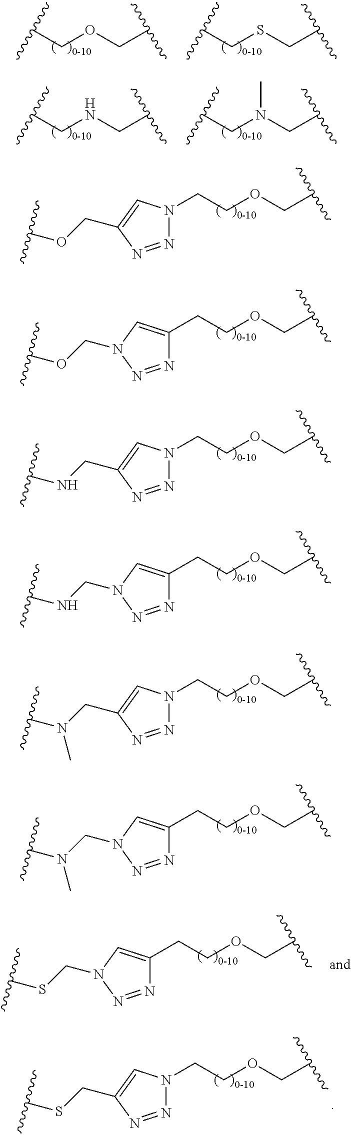

In certain embodiments, in the compound of Formula II, Linker^{B} is selected from:

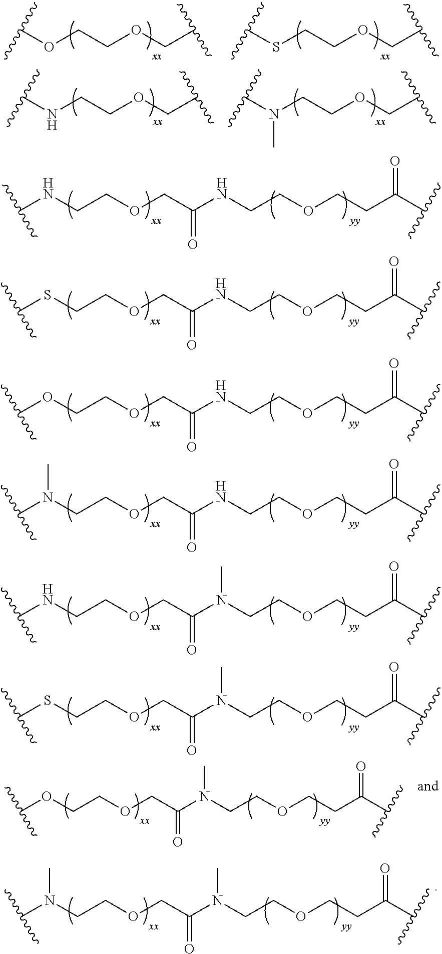

In certain embodiments, in the compound of Formula II, Linker^{B} is selected from:

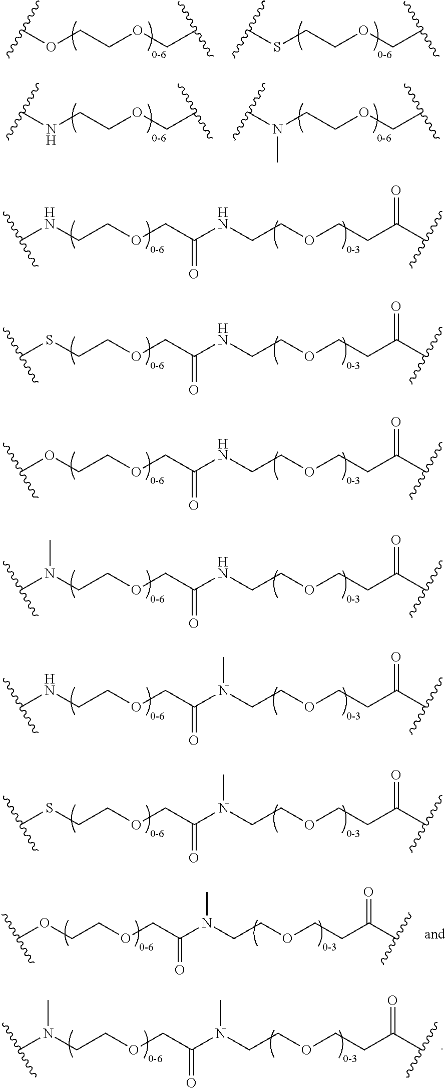

In certain embodiments, in the compound of Formula II, Linker^{B} is selected from:

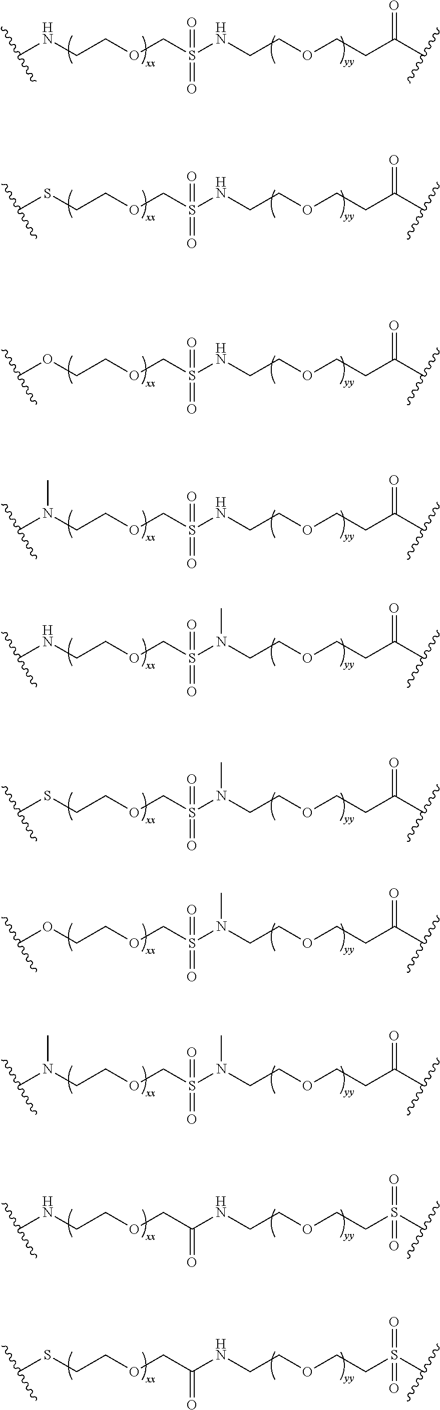

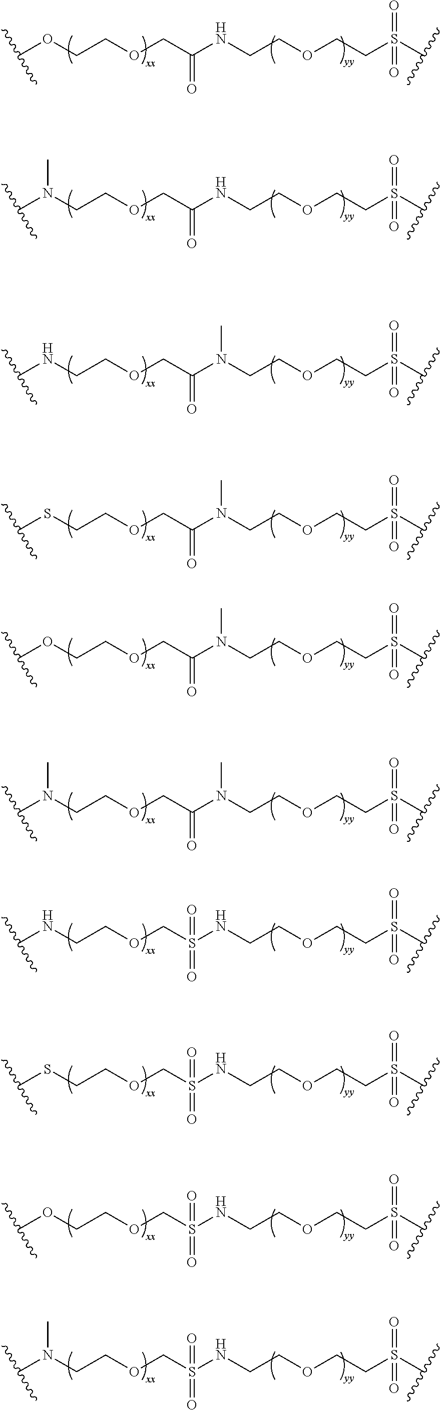

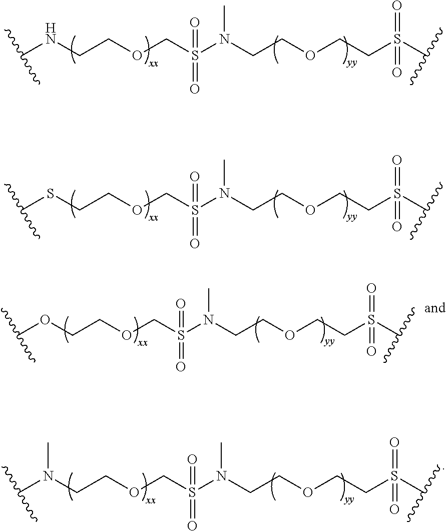

In certain embodiments, in the compound of Formula II, Linker^{B} is selected from:

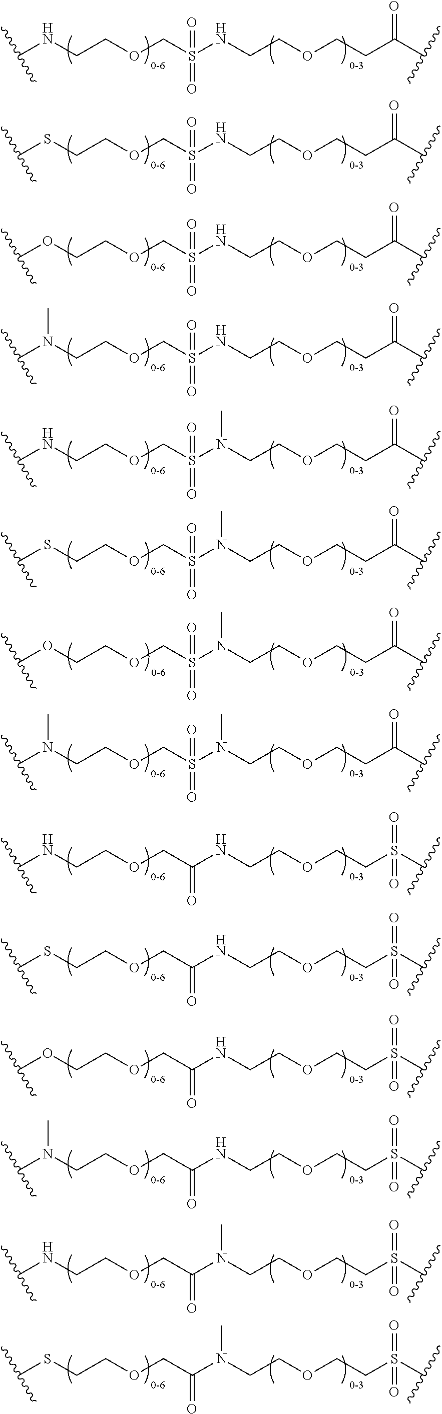

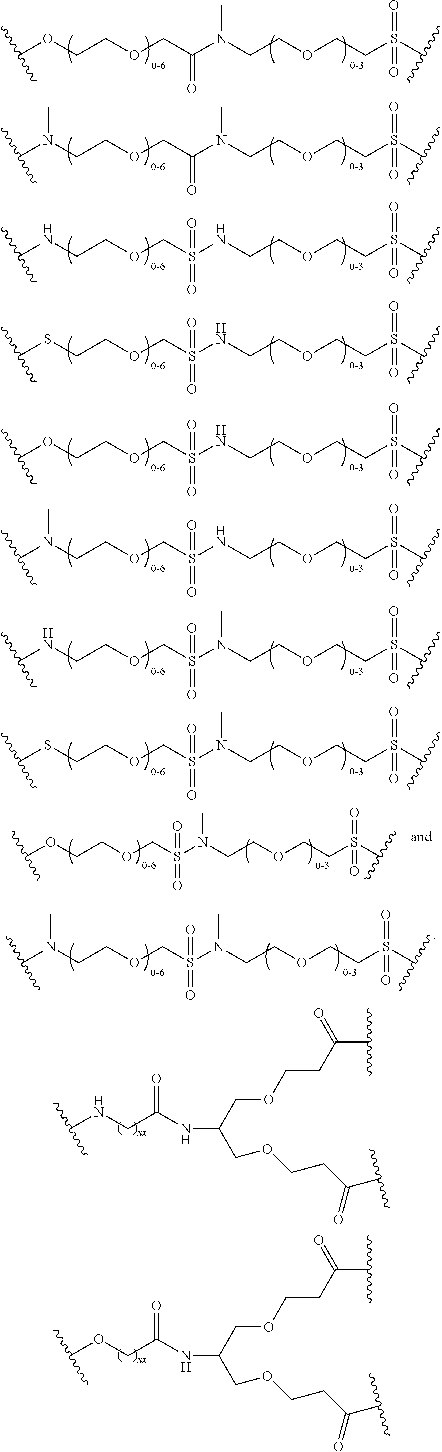

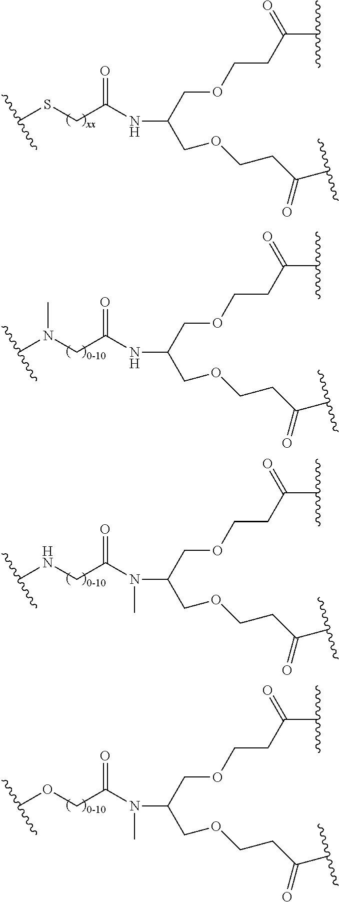

In certain embodiments, in the compound of Formula II, Linker^{C} is selected from:

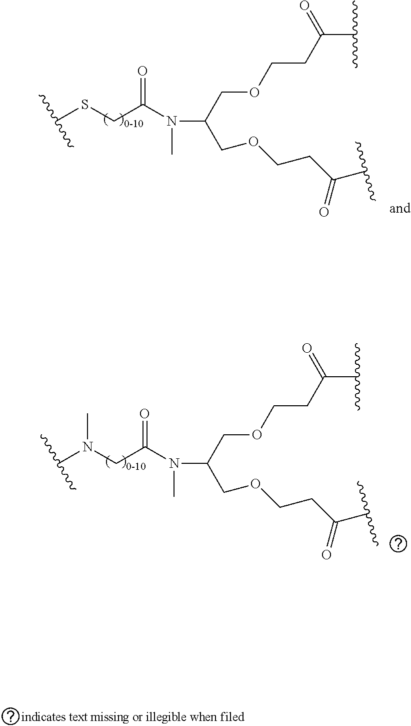

In certain embodiments, in the compound of Formula II, Linker^{C} is selected from:

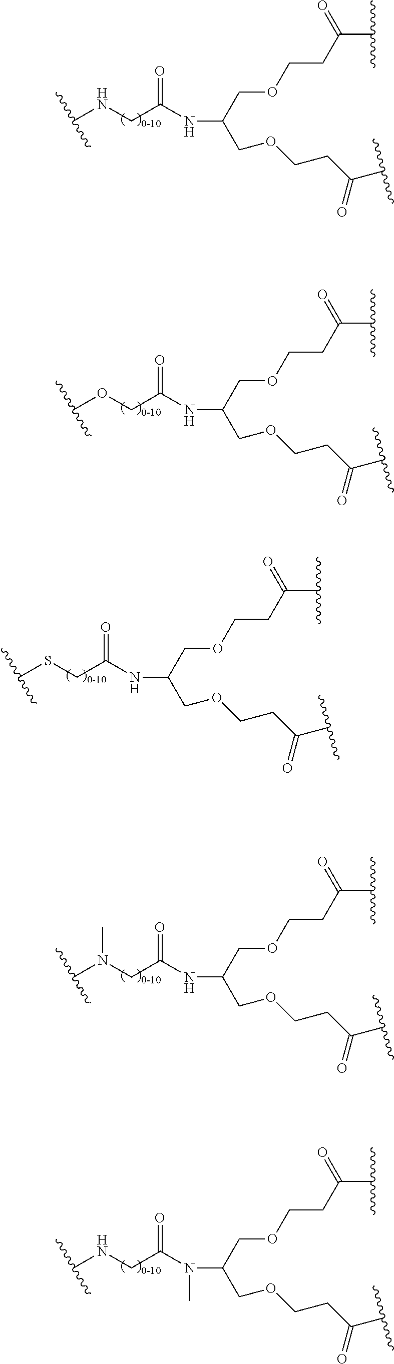

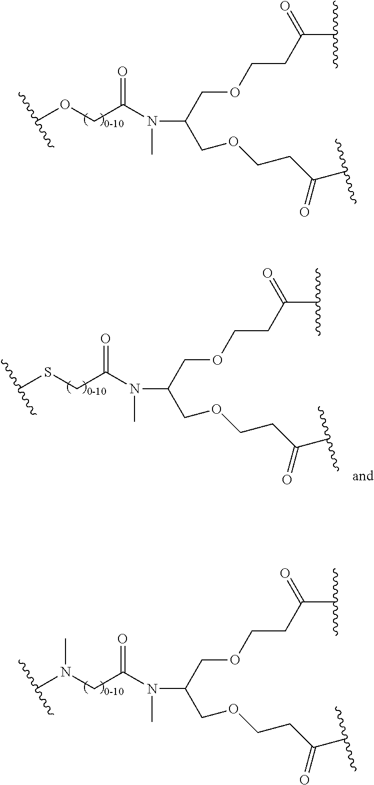

In certain embodiments, in the corn und of Formula II, Linker^{C} is selected from:

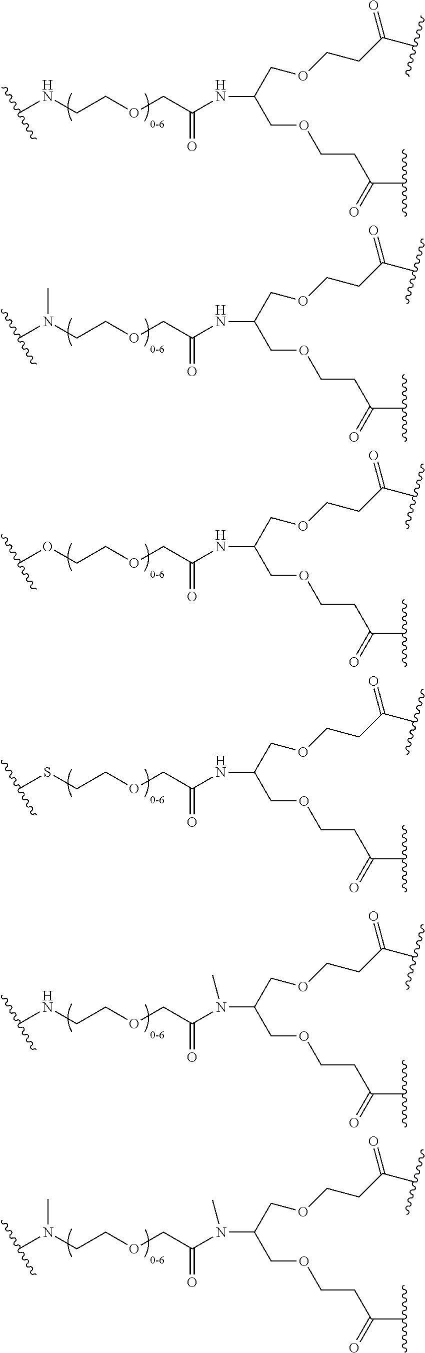

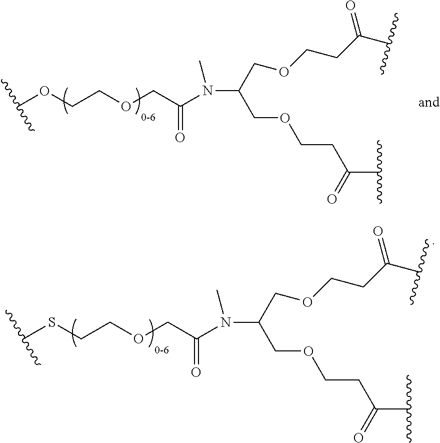

In certain embodiments, in the compound of Formula II, Linker^{C} is selected from:

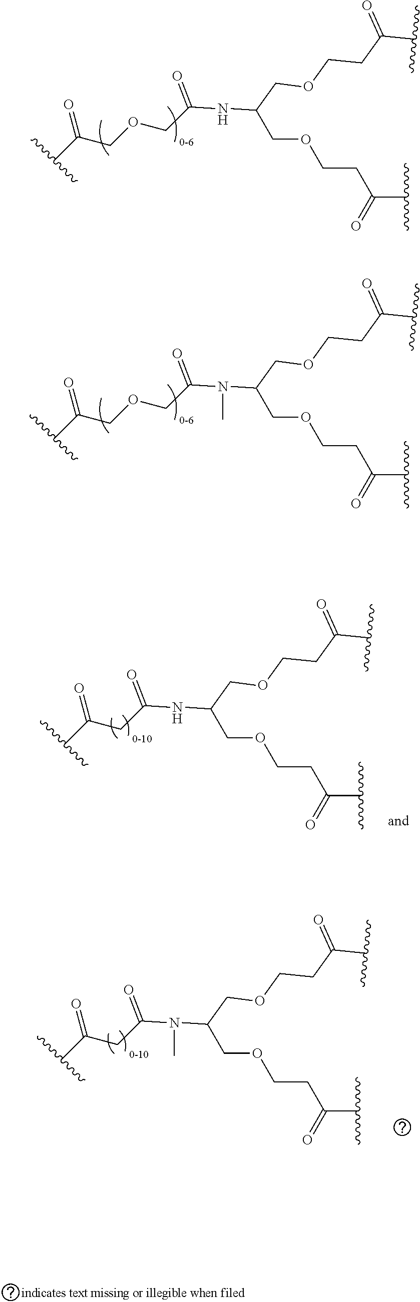

In certain embodiments, in the compound of Formula II, Linker^{C} is selected from:

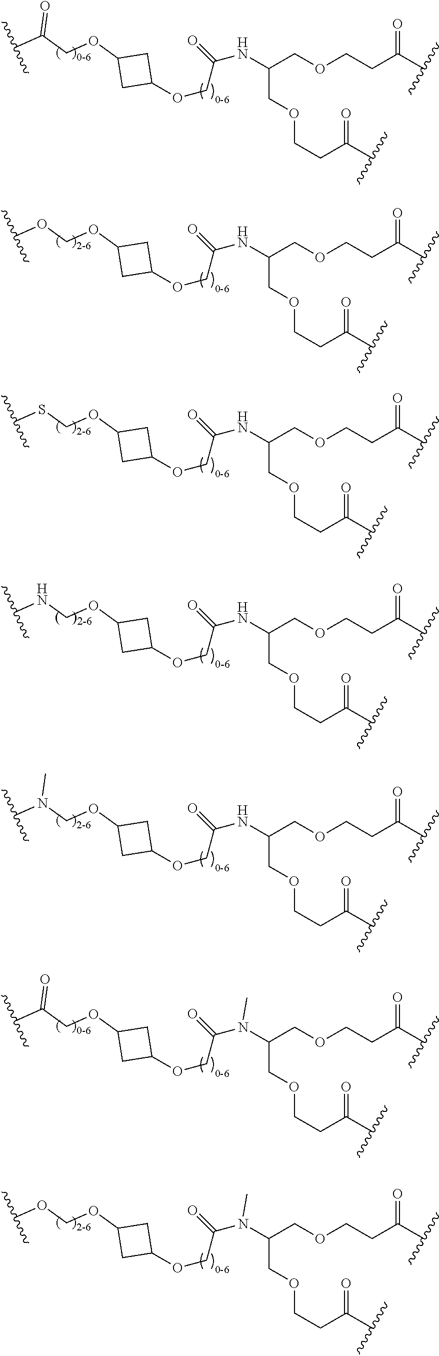

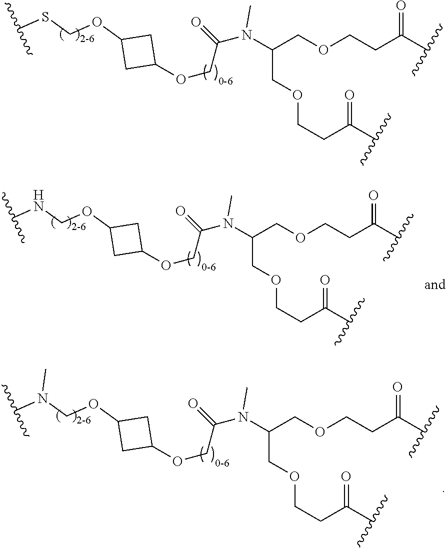

In certain embodiments, in the compound of Formula II, Linker^{C} is selected from:

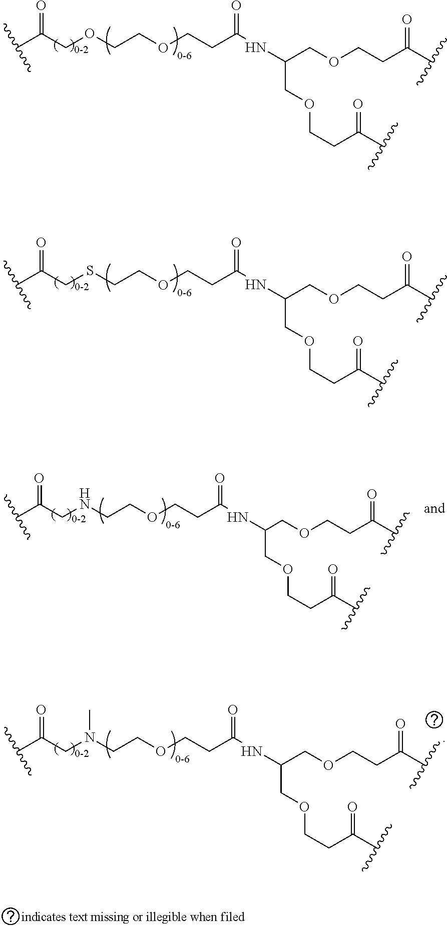

In certain embodiments, in the compound of Formula II, Linker^{C} is selected from:

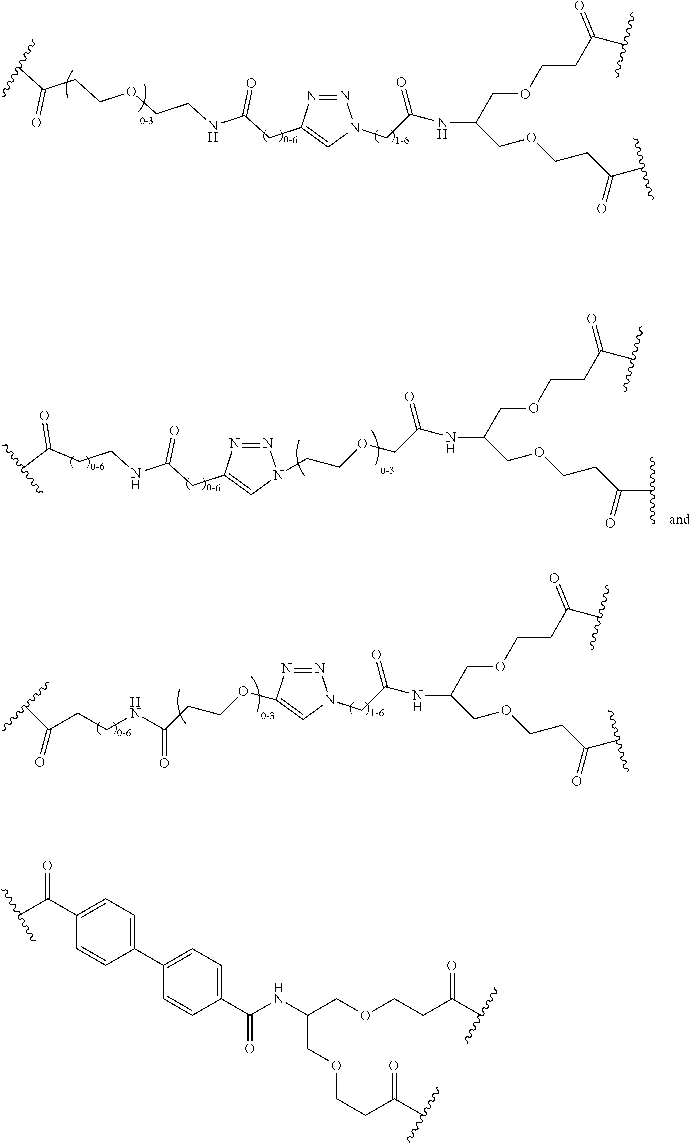

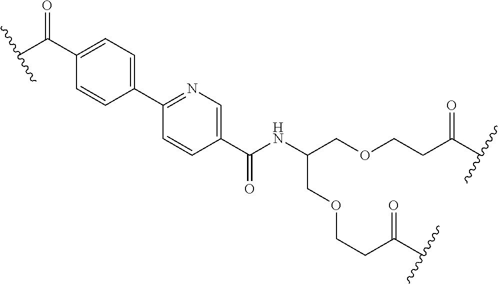 [SMILES: *C(=O)CCOCC(COCCC(*)=O)NC(=O)c1ccc(-c2ccc(C(*)=O)cc2)nc1]

In certain embodiments, in the compound of Formula II, Linker^{C} is selected from:

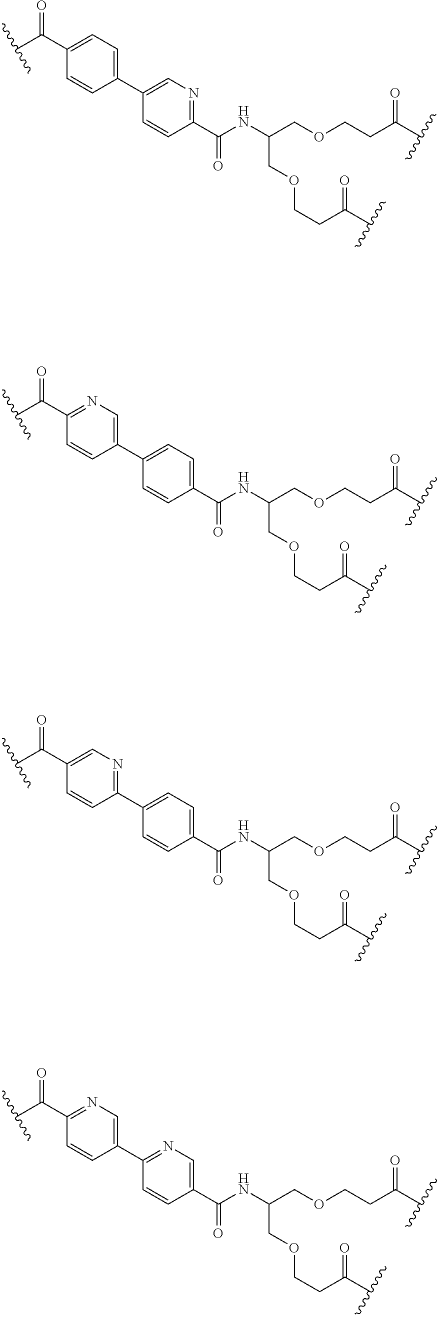 [SMILES: *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1 ; *C(=O)CCOCC(COCCC(*)=O)NC(=O)c1ccc(-c2ccc(C(*)=O)cn2)cc1 ; *C(=O)CCOCC(COCCC(*)=O)NC(=O)c1ccc(-c2ccc(C(*)=O)nc2)nc1]

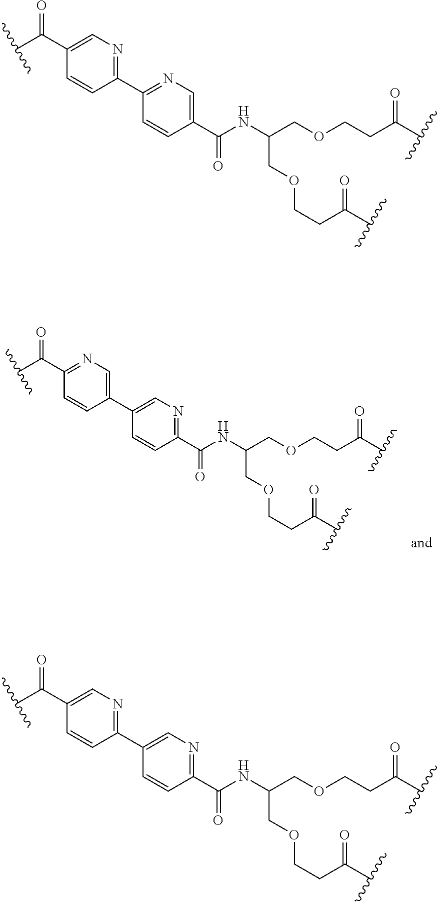 [SMILES: *C(=O)CCOCC(COCCC(*)=O)NC(=O)c1ccc(-c2ccc(C(*)=O)cn2)nc1 ; *C(=O)CCOCC(COCCC(*)=O)NC(=O)c1ccc(-c2ccc(C(*)=O)nc2)cn1 ; *C(=O)CCOCC(COCCC(*)=O)NC(=O)c1ccc(-c2ccc(C(*)=O)cn2)cn1]

In certain embodiments, in the compound of Formula II, Linker^{D} is selected from:

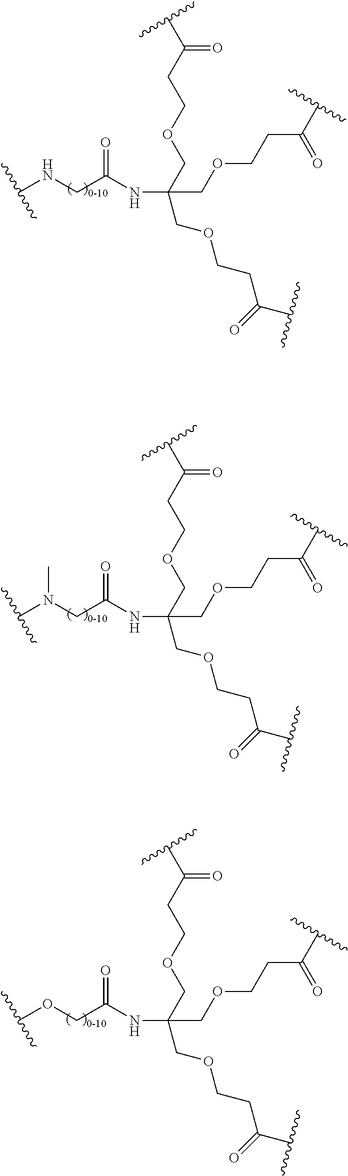

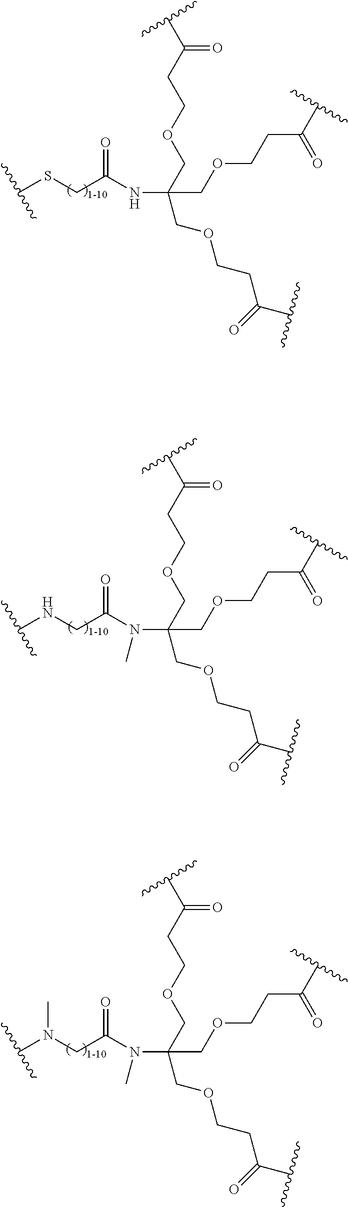

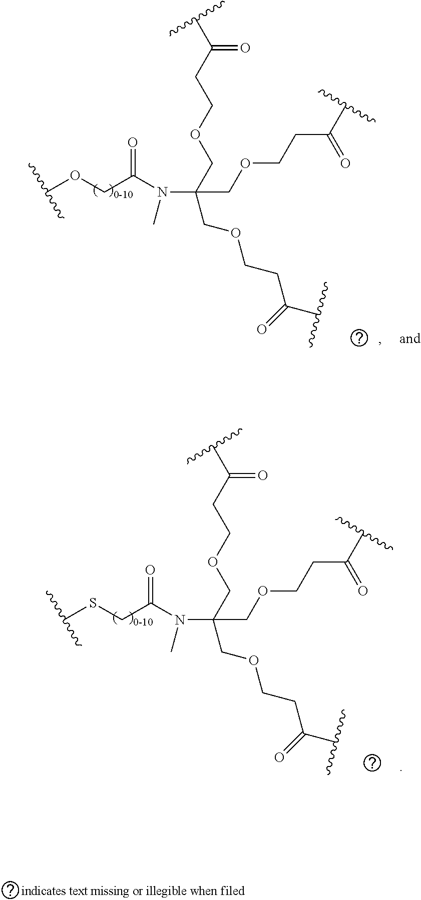

In certain embodiments, in the compound of Formula II, Linker^{D} is selected from:

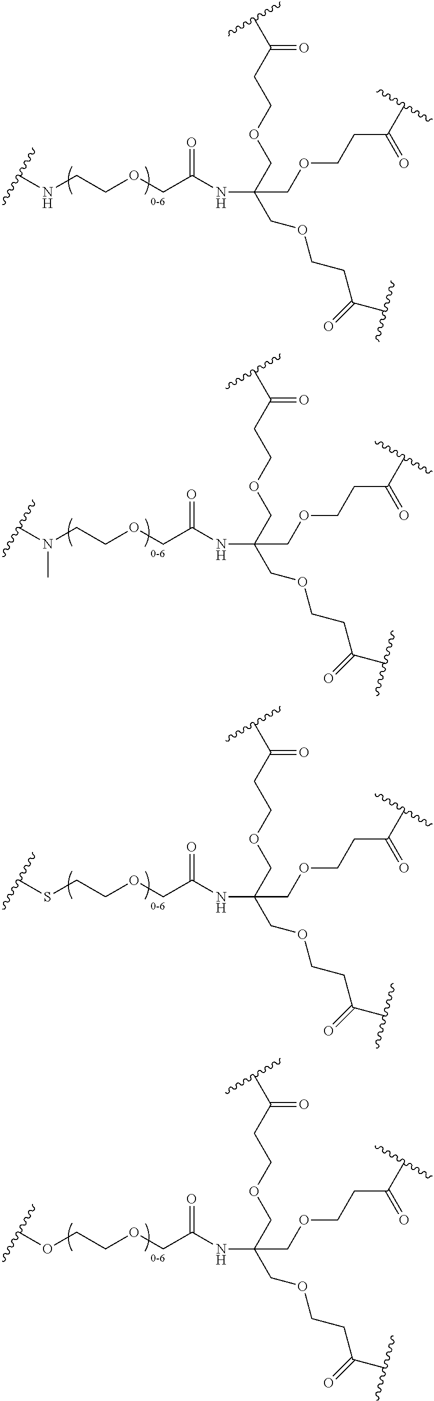

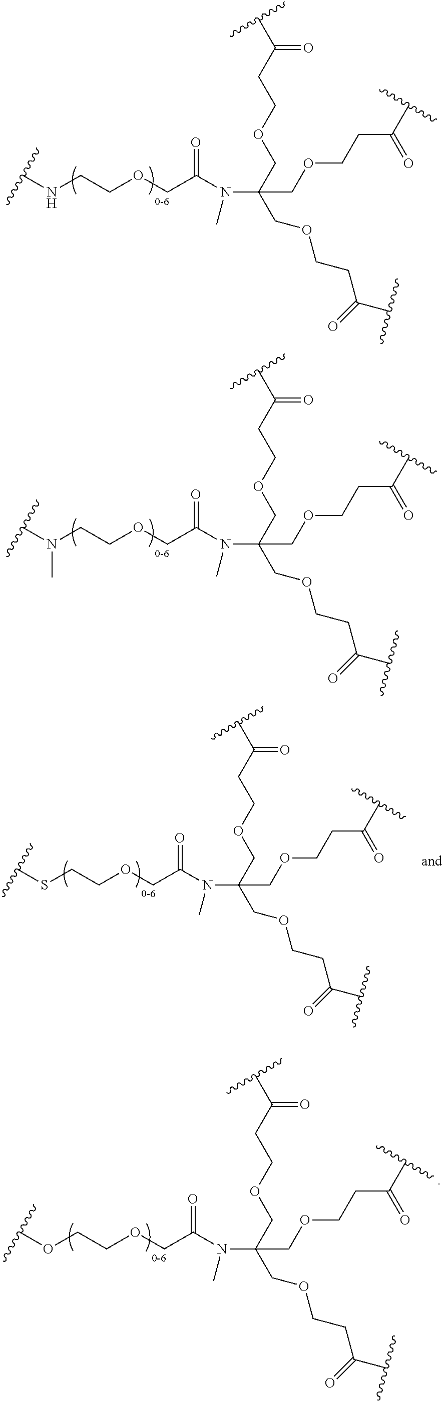

In certain embodiments, in the compound of Formula II, Linker^{D} is selected from:

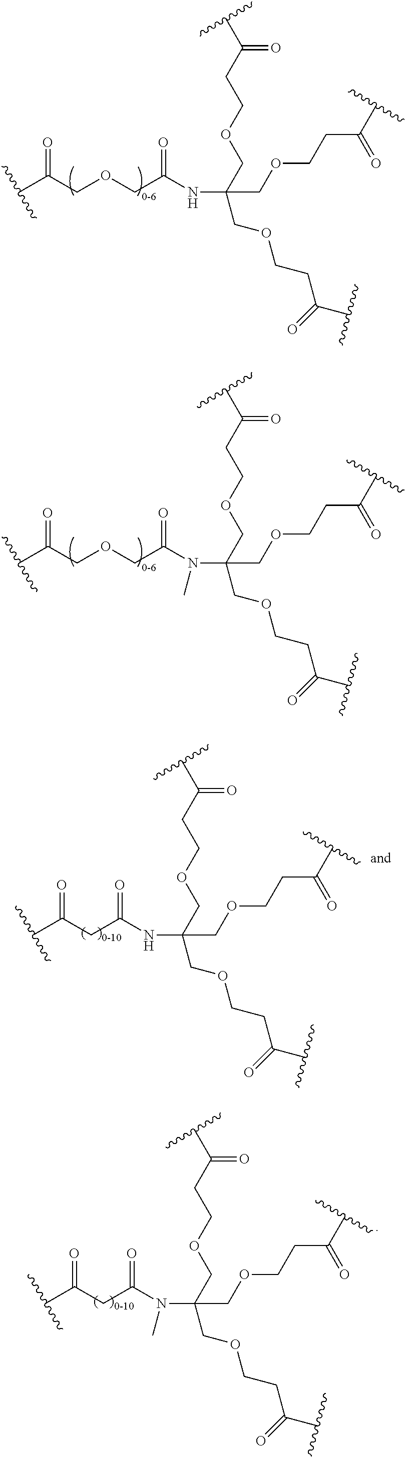

In certain embodiments, in the compound of Formula II, Linker^{D} is selected from:

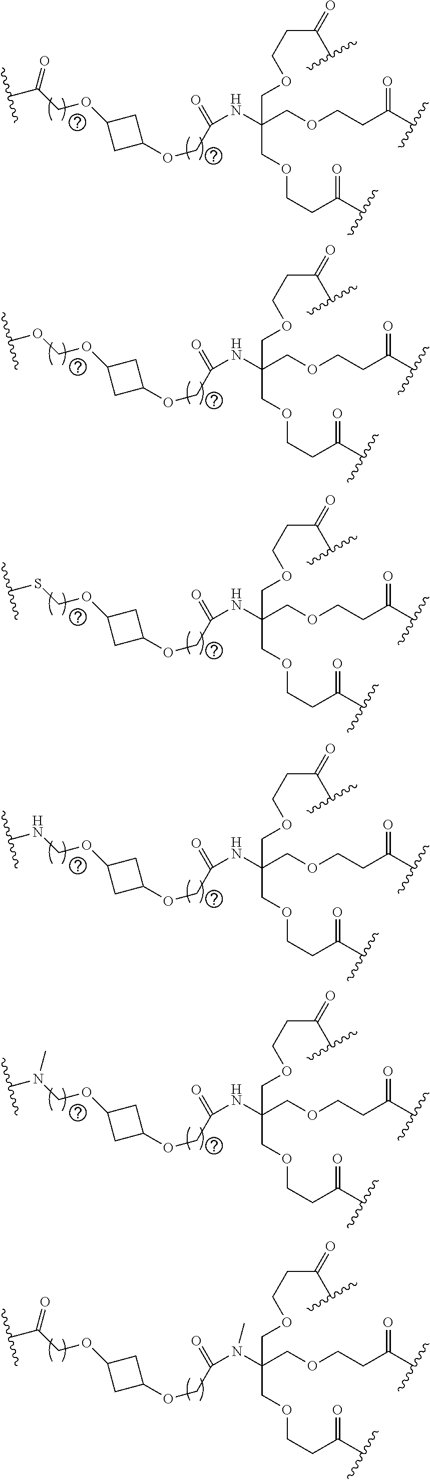

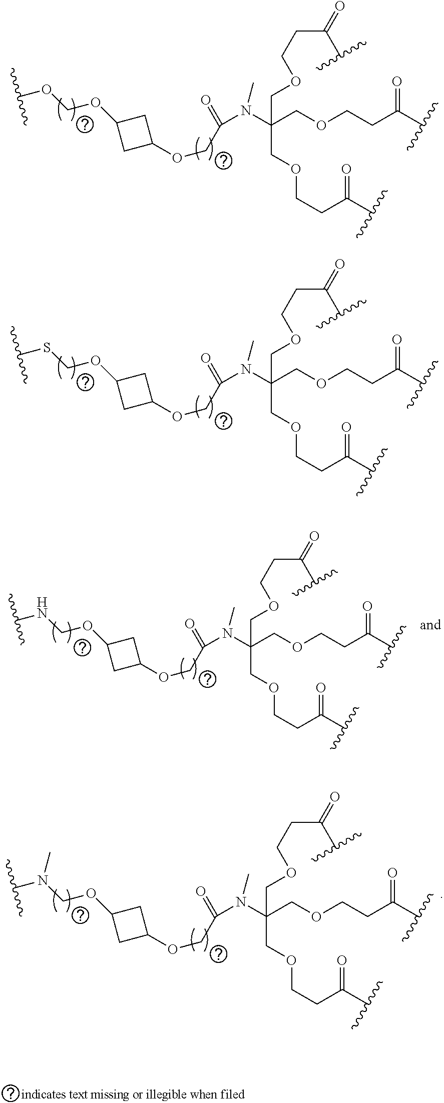

In certain embodiments, in the compound of Formula IL, Linker^{D} is selected from:

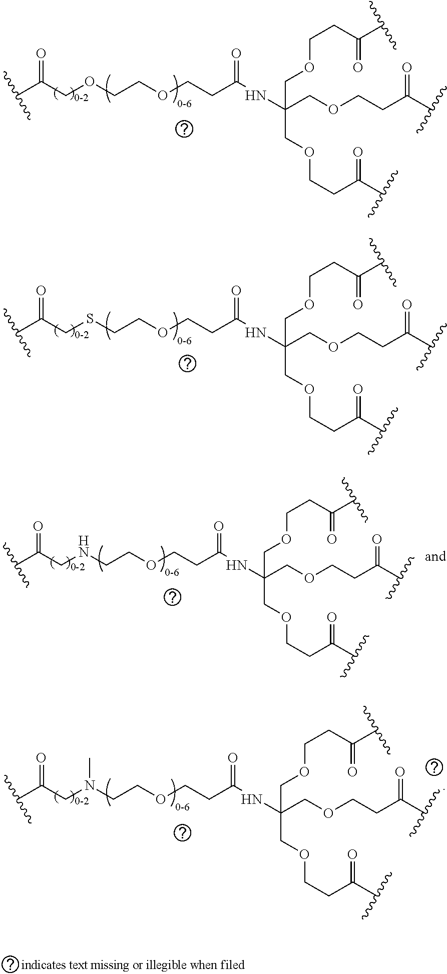

In certain embodiments, in the compound of Formula II, Linker^{D} is selected from:

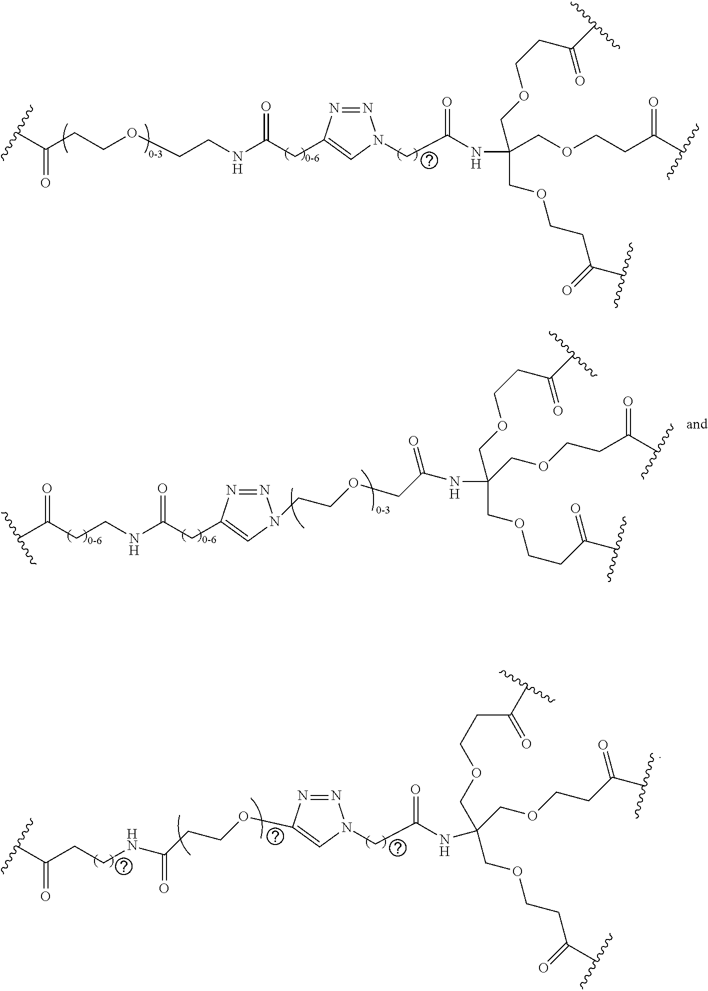

In certain embodiments, in the compound of Formula II, Linker^{D} is selected from:

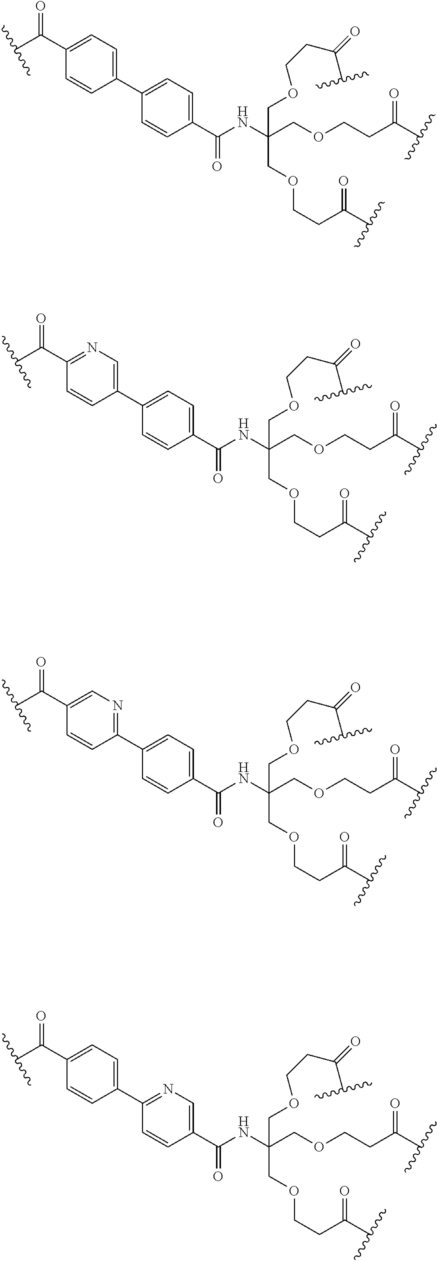 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1]

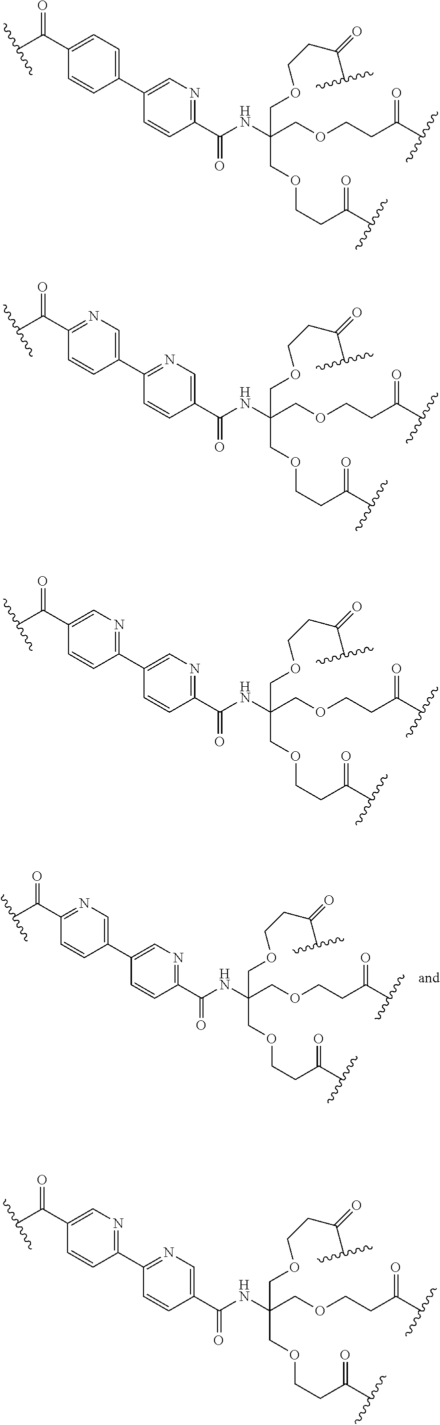 [SMILES: *C(=O)CCOCC(COCCC(*)=O)(COCCC(*)=O)NC(=O)c1ccc(-c2ccc(C(*)=O)cc2)cn1 ; *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1 ; *C(=O)CCOCC(COCCC(*)=O)(COCCC(*)=O)NC(=O)c1ccc(-c2ccc(C(*)=O)cn2)nc1]

In certain embodiments, in the compound of Formula II, the Linker^{A} is selected from

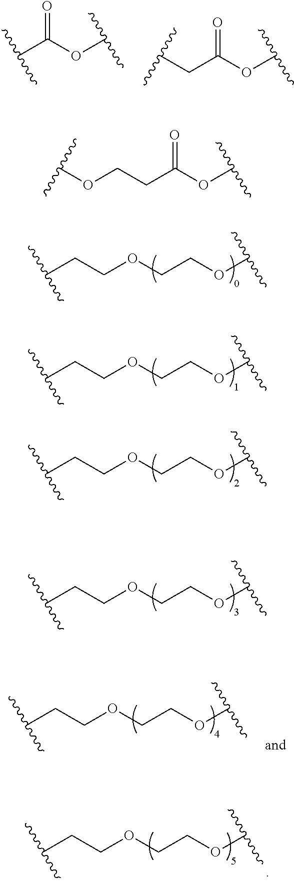

In certain embodiments, in the compound of Formula II, the Linker^{A} is selected from

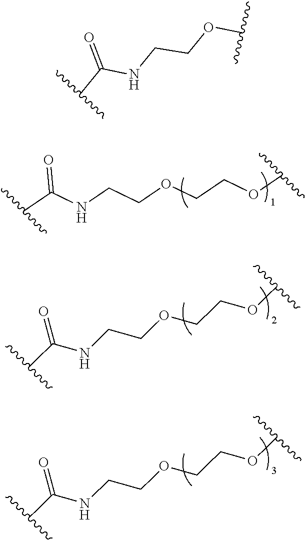

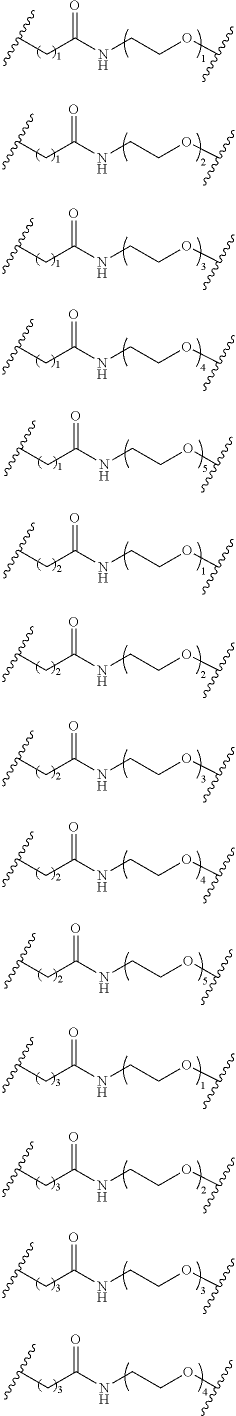

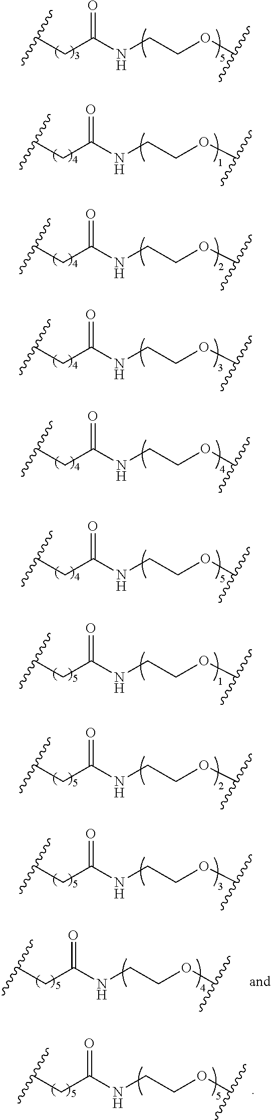

In certain embodiments, the Linker^{A} is selected from

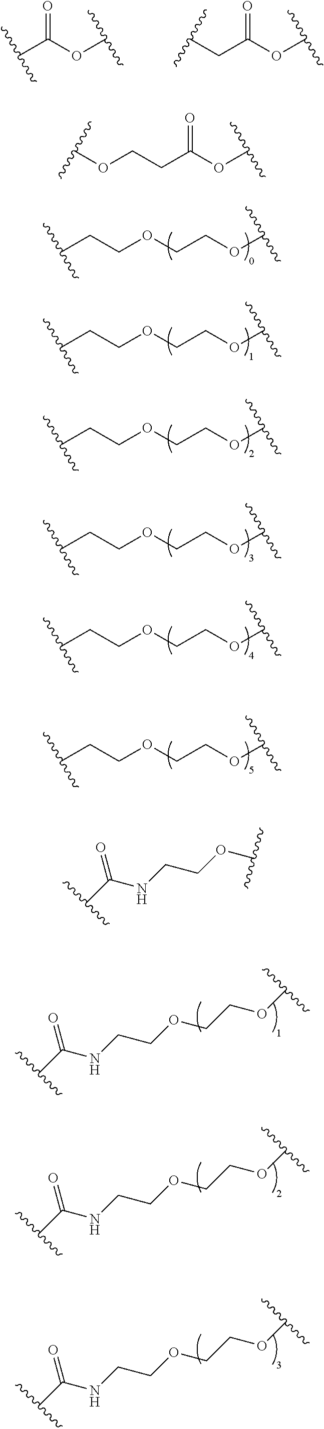

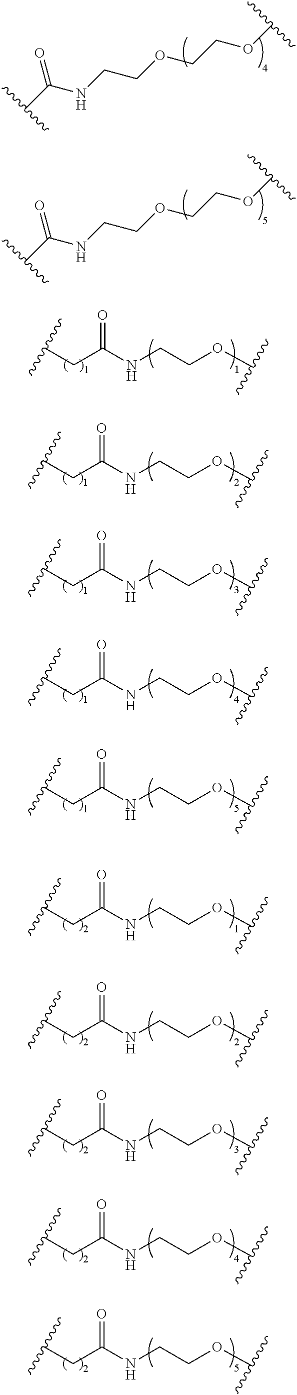

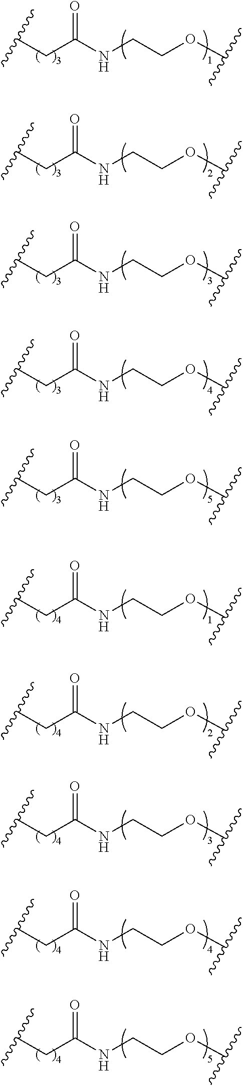

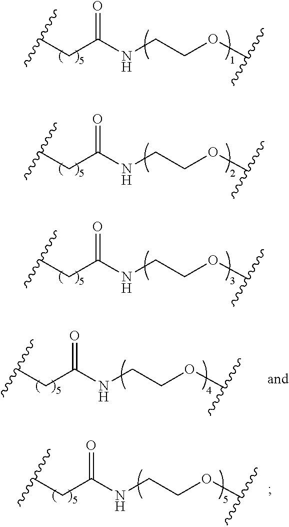

wherein each is optionally substituted with 1, 2, 3, or 4 substituents substituent selected from R²¹.

In certain embodiments, in the compound of Formula II, Linker^{A} is selected from:

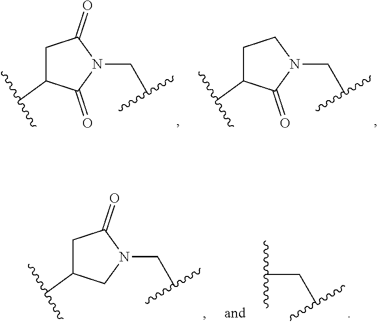

In certain embodiments, in the compound of Formula II, the Linker^{A} is selected from

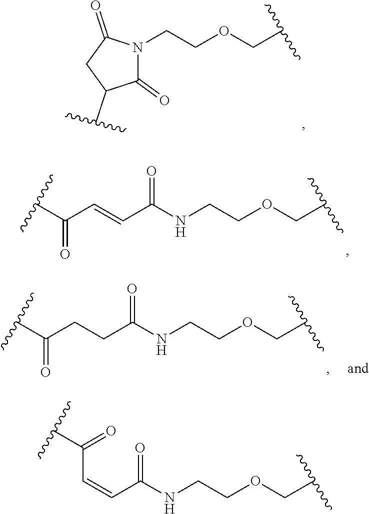 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments, in the compound of Formula II, the Linker^{A} is selected from

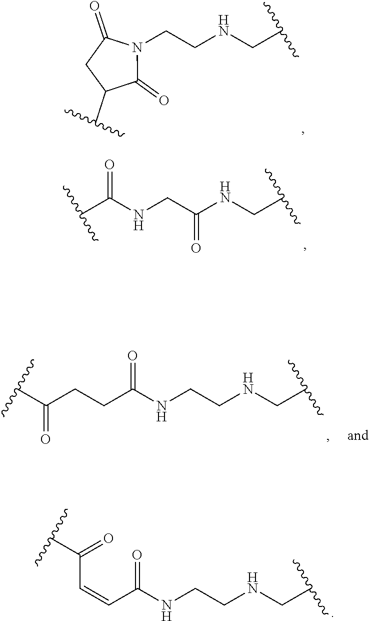

In certain embodiments, in the compound of Formula II, the Linker^{A} is selected from

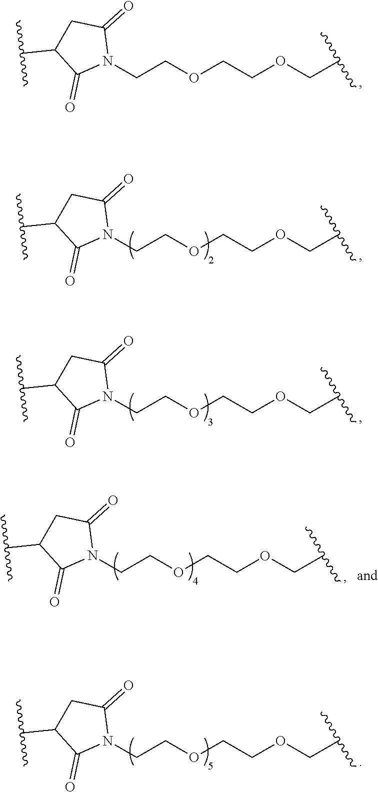

In certain embodiments, in the compound of Formula II, the Linker^{A} is selected from

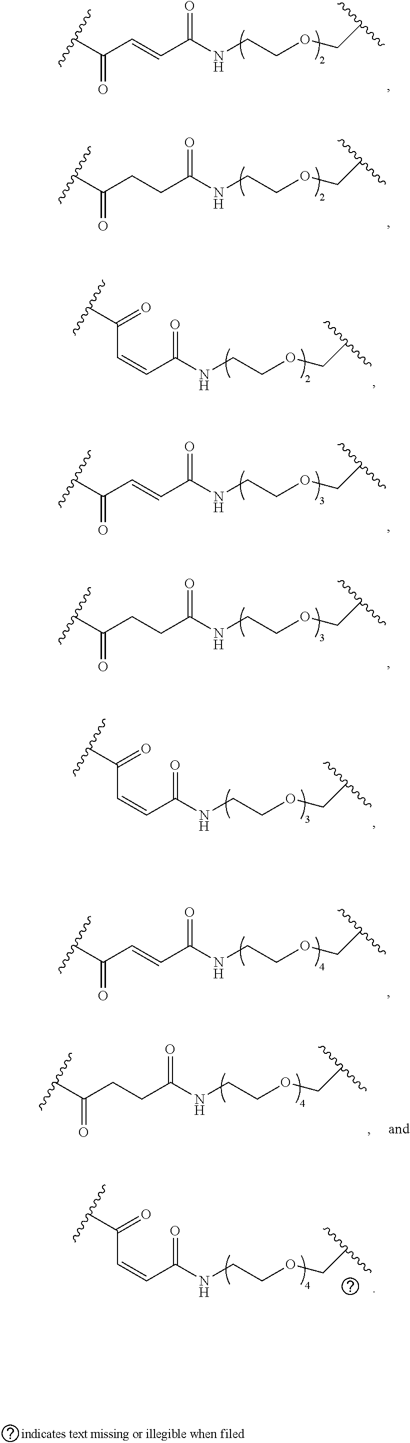

In certain embodiments, in the compound of Formula II, the Linker^{A} is selected from

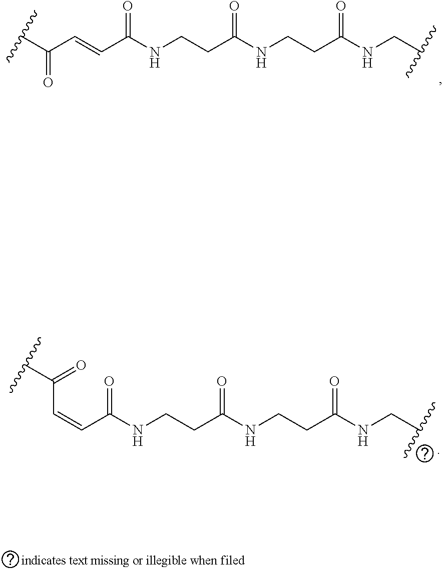 [SMILES: *CNC(=O)CCNC(=O)CCNC(=O)/C=C/C(*)=O ; *CNC(=O)CCNC(=O)CCNC(=O)/C=C\C(*)=O]

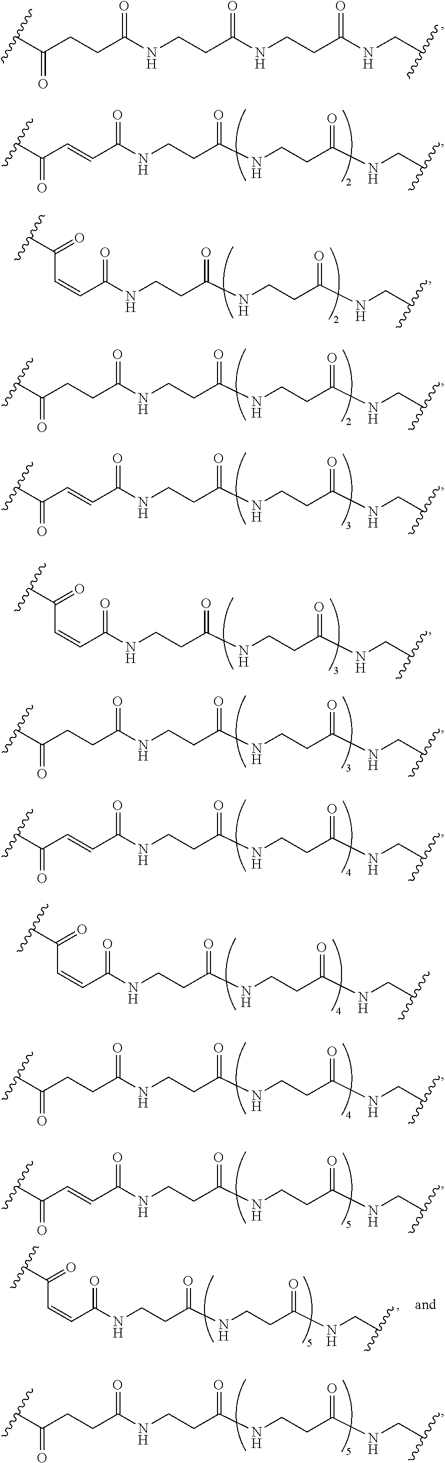

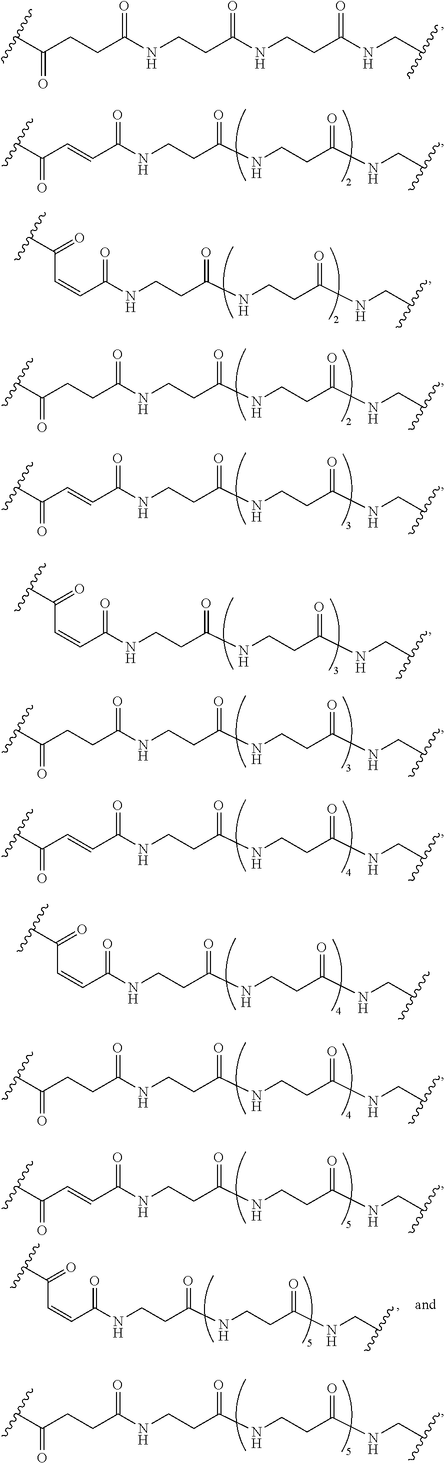

In certain embodiments, in the compound of Formula II, Linker^{A} is selected from:

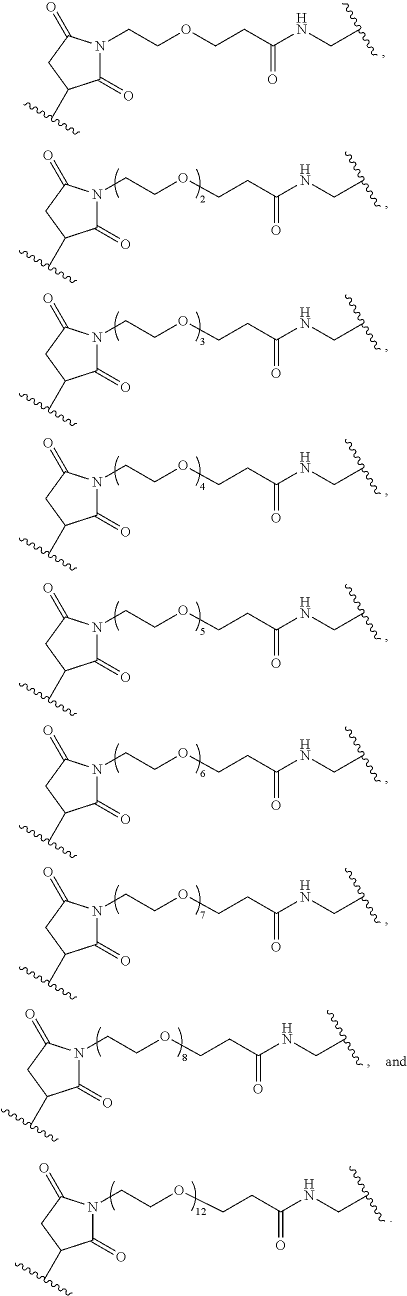

In certain embodiments, in the compound of Formula II, the Linker^{A} is selected from

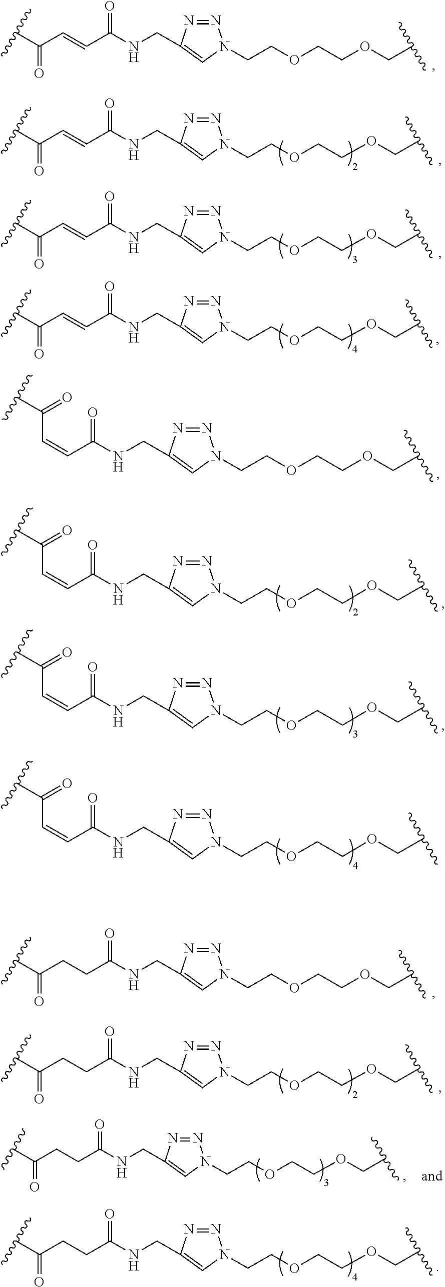

In certain embodiments, in the compound of Formula II, the Linker^{A} is selected from

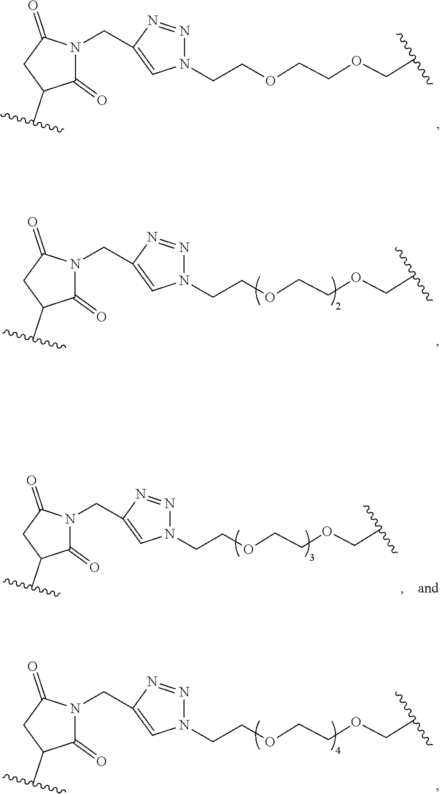

In certain embodiments, in the compound of Formula II, the Linker^{A} is selected from

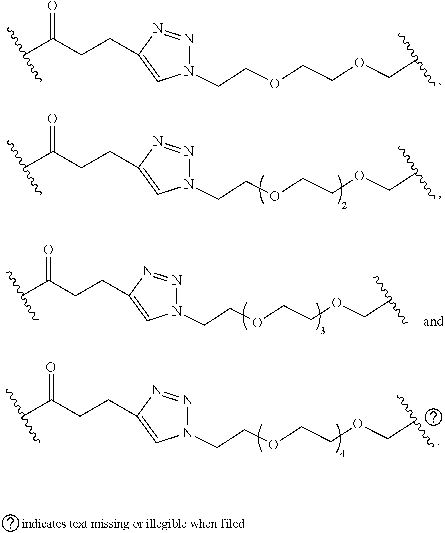

In certain embodiments, in the compound of Formula II, the Linker^{A} is selected from

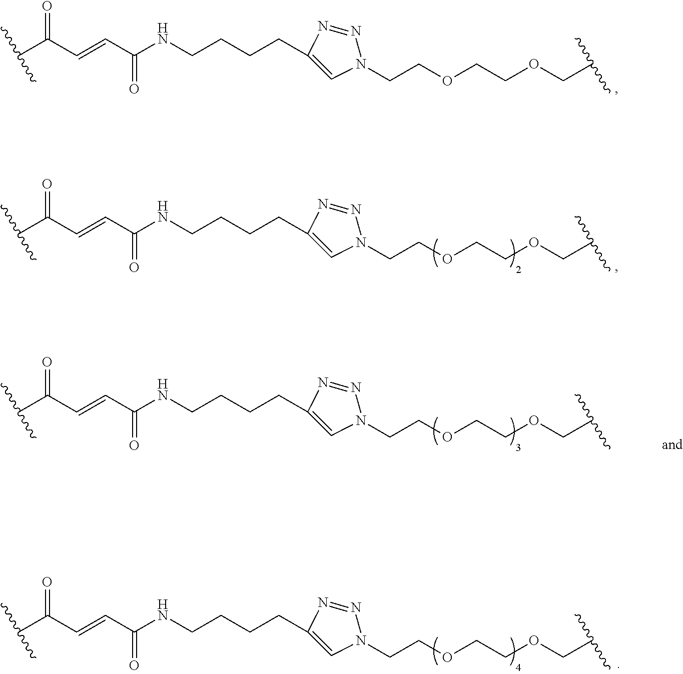

In certain embodiments, in the compound of Formula II, the Linker^{A} is selected from

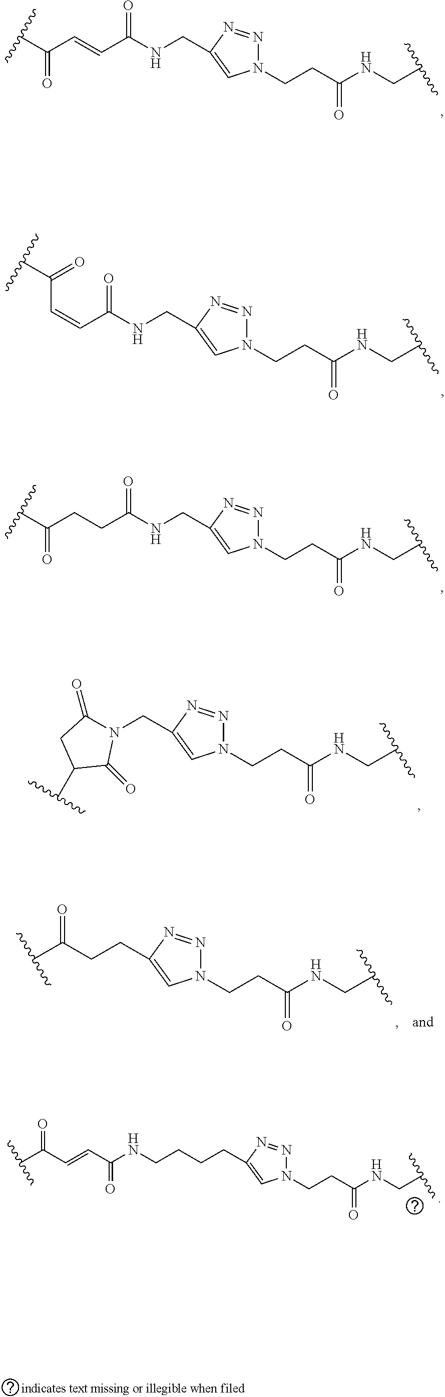 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CCC(*)=O)nn1 ; *CNC(=O)CCn1cc(CCCCNC(=O)/C=C/C(*)=O)nn1]

In certain embodiments, in the compound of Formula II, the Linker^{A} is selected from

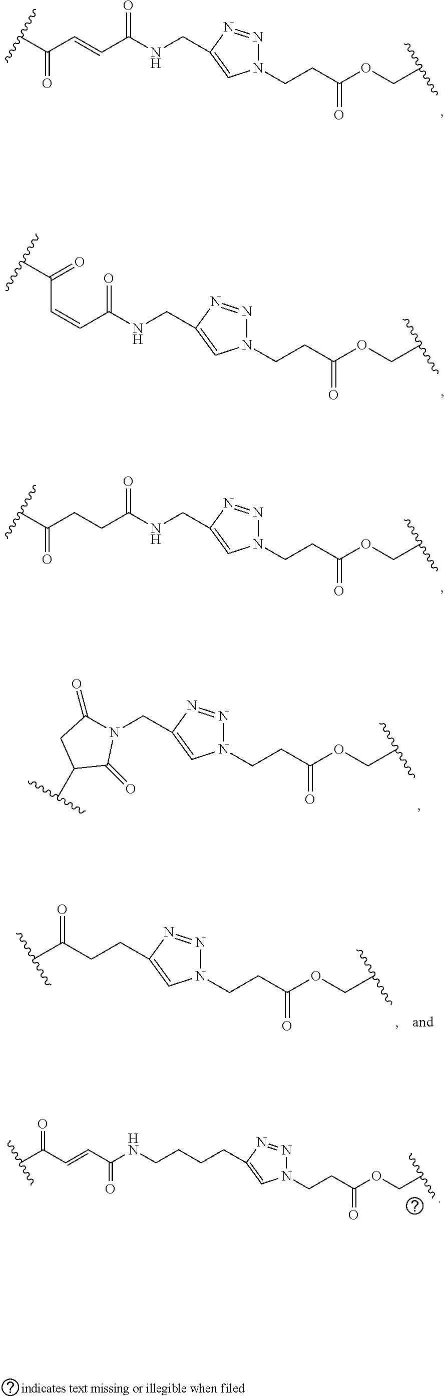 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CCC(*)=O)nn1 ; *COC(=O)CCn1cc(CCCCNC(=O)/C=C/C(*)=O)nn1]

In certain embodiments, in the compound of Formula II, the Linker^{B} is selected from

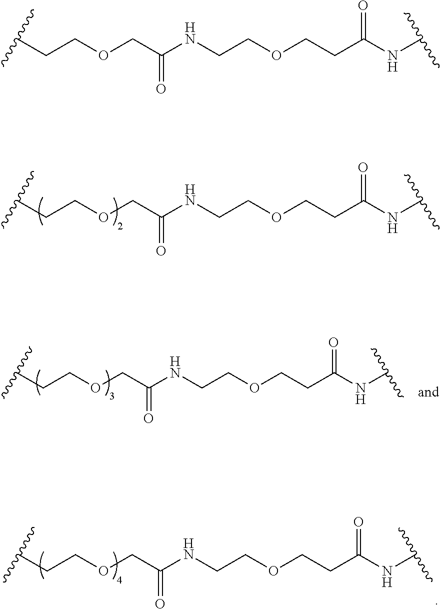

In certain embodiments, in the compound of Formula II, the Linker^{B} is selected from

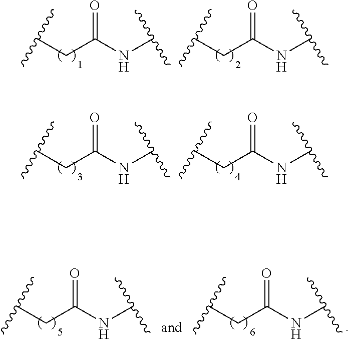

In certain embodiments, in the compound of Formula II, the Linker^{B} is selected from

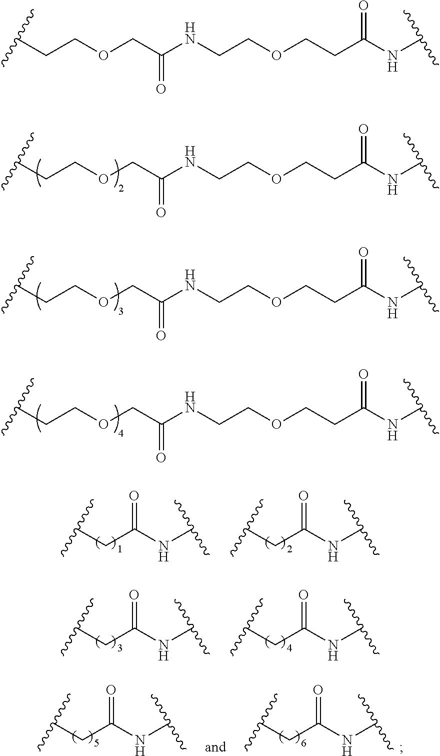

In certain embodiments, in the compound of Formula II, the Linker^{B} is selected from wherein each is optionally substituted with 1, 2, 3, or 4 substituents substituent selected from R²¹.

In certain embodiments, in the compound of Formula II Linker^{B} is selected from:

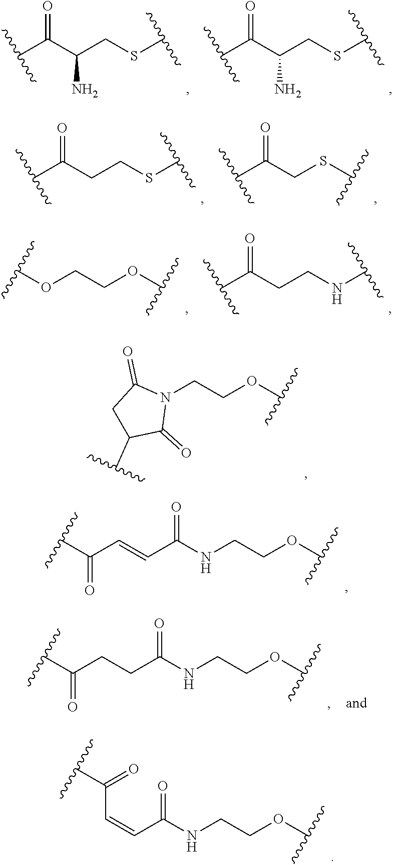

In certain embodiments, in the compound of Formula II, the Linker^{B} is selected from:

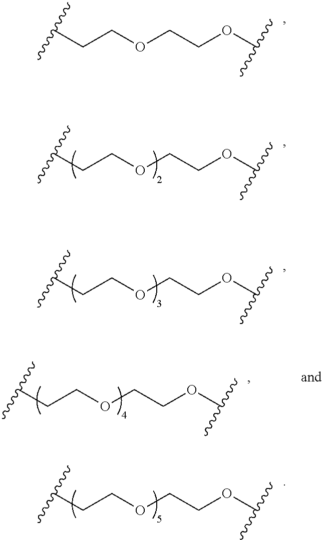

In certain embodiments, in the compound of Formula II, the Linker^{B} is selected from:

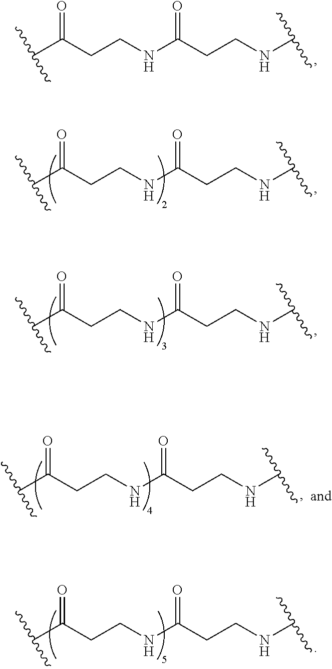

In certain embodiments, in the compound of Formula II, the Linker^{B} is selected from:

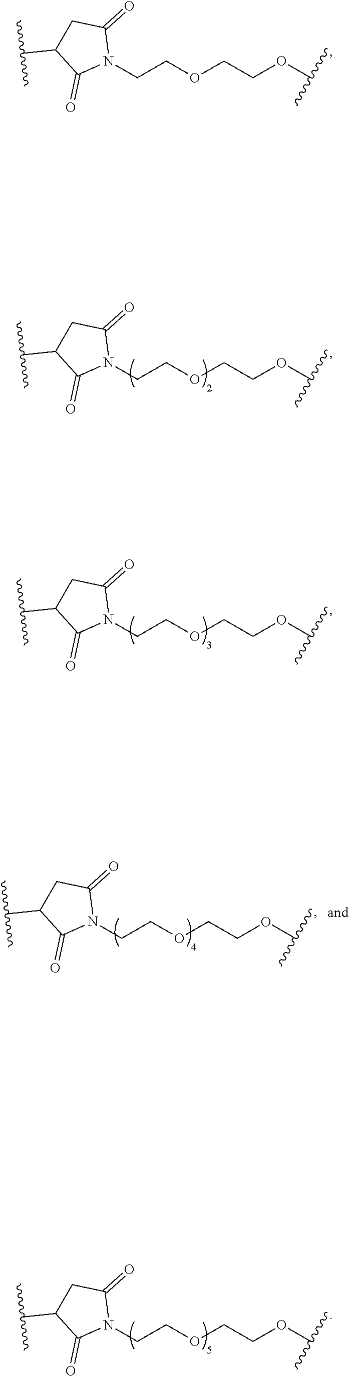

In certain embodiments, in the compound of Formula II, the Linker^{B} is selected from:

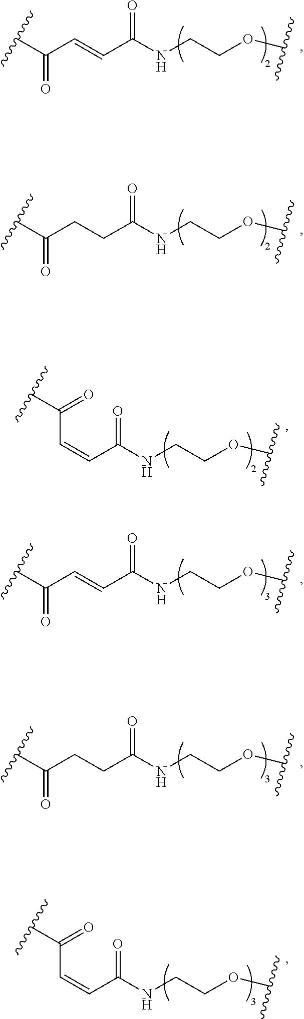

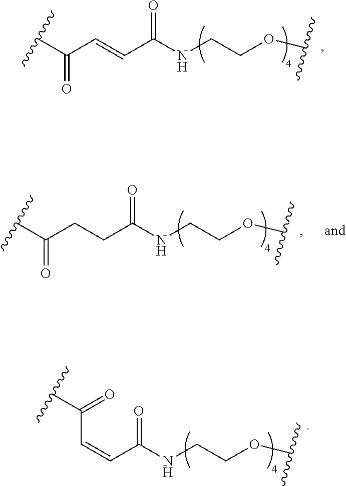

In certain embodiments, in the compound of Formula II, the Linker^{B} is selected from:

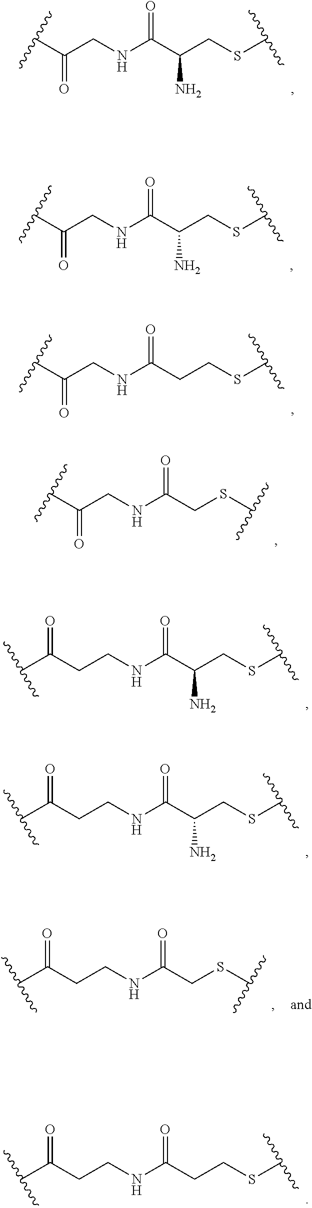

In certain embodiments, in the compound of Formula II, the Linker^{B} is selected from:

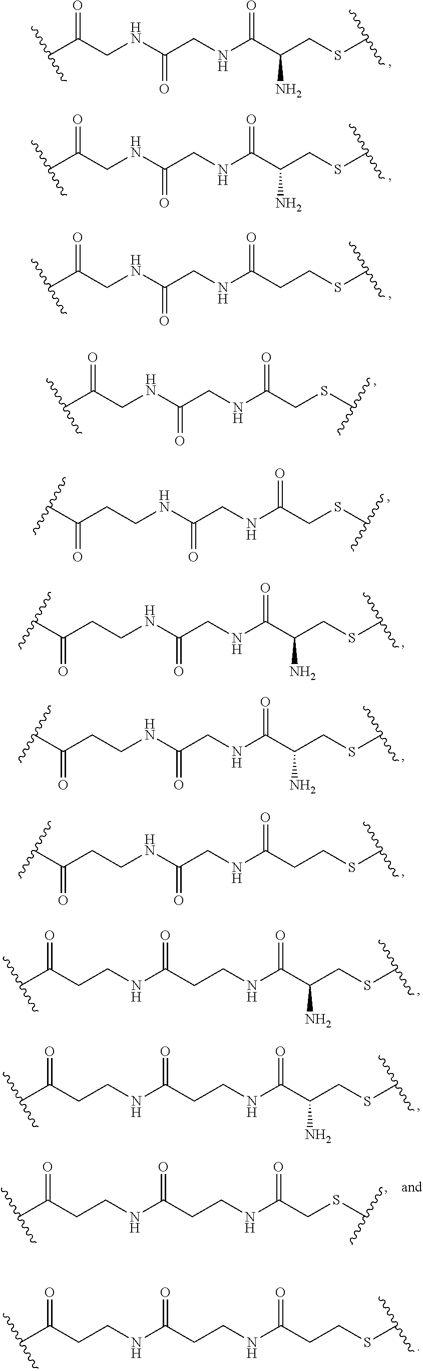

In certain embodiments, in the compound of Formula II, the Linker^{B} is selected from:

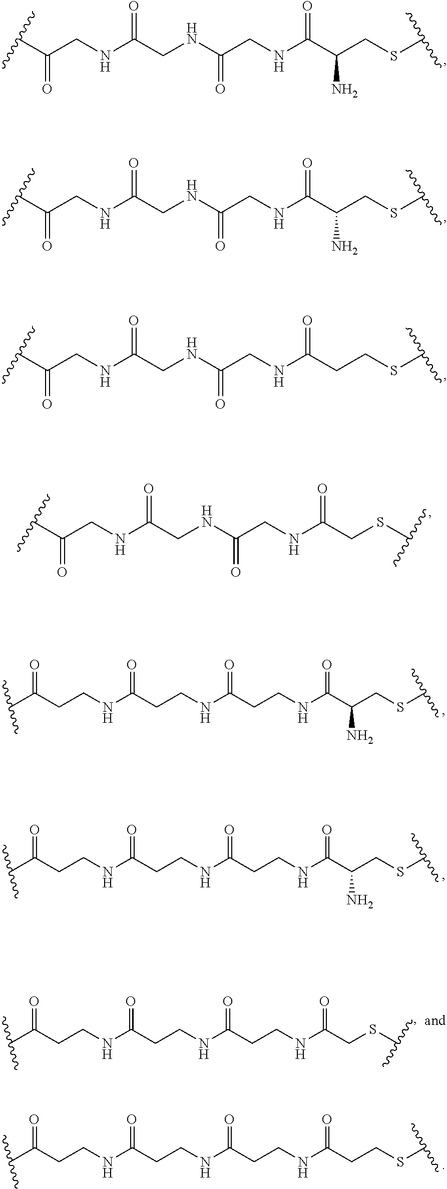

In certain embodiments, in the compound of Formula II, Linker^{B}-Linker^{A} is selected

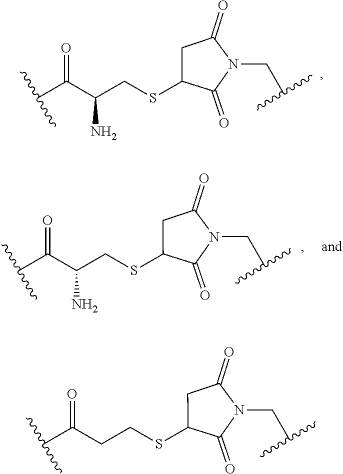 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments, in the compound of Formula II, Linker^{B}-Linker^{A} is selected from:

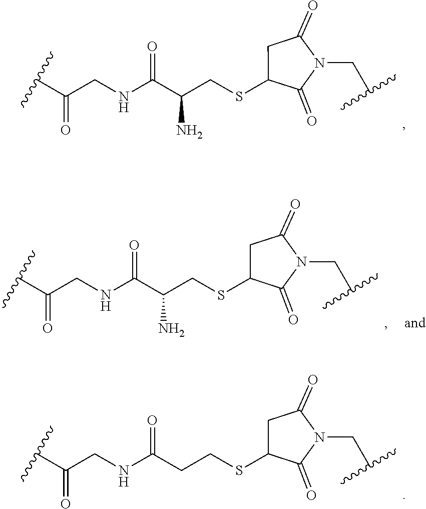 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, in the compound of Formula II, the Linker^{C} is selected from:

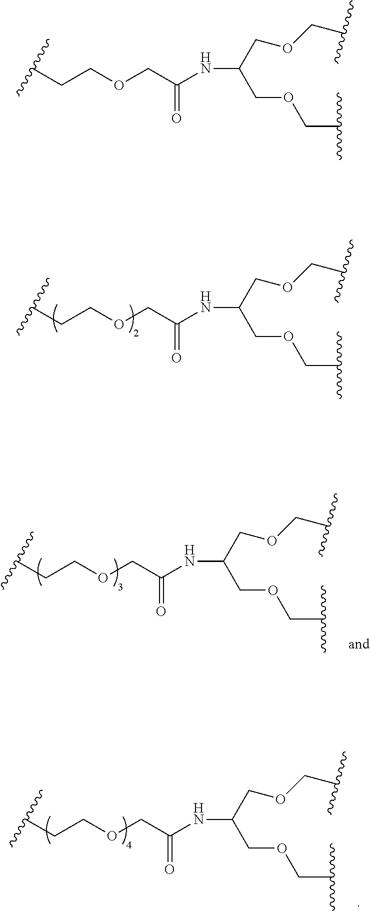

In certain embodiments, in the compound of Formula II, the Linker^{C} is selected from:

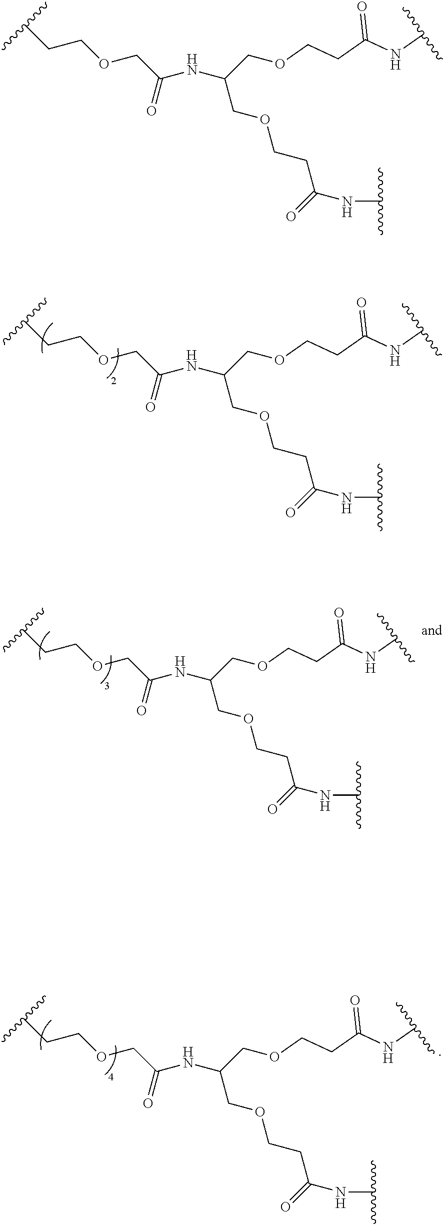

In certain embodiments, in the compound of Formula II, the Linker^{C} is selected from:

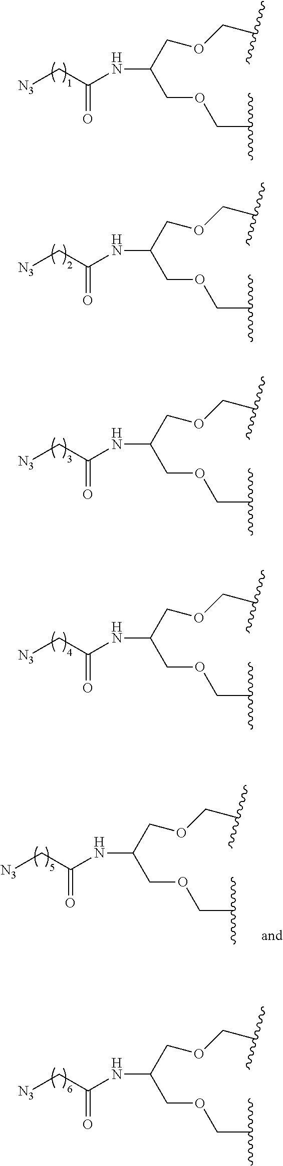

In certain embodiments, in the compound of Formula II, the Linker^{C} is selected from:

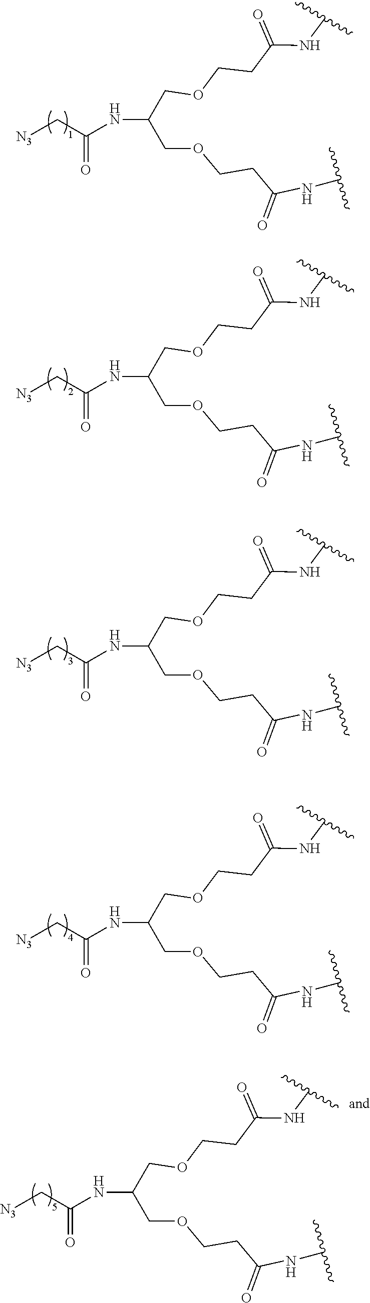

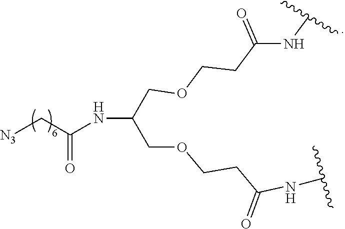

In certain embodiments, in the compound of Formula II, the Linker^{C} is selected from:

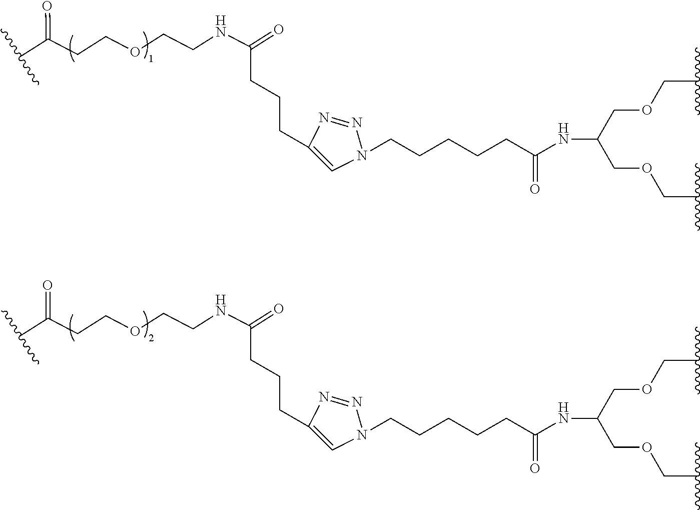

In certain embodiments, in the compound of Formula II, the Linker^{C} is selected from:

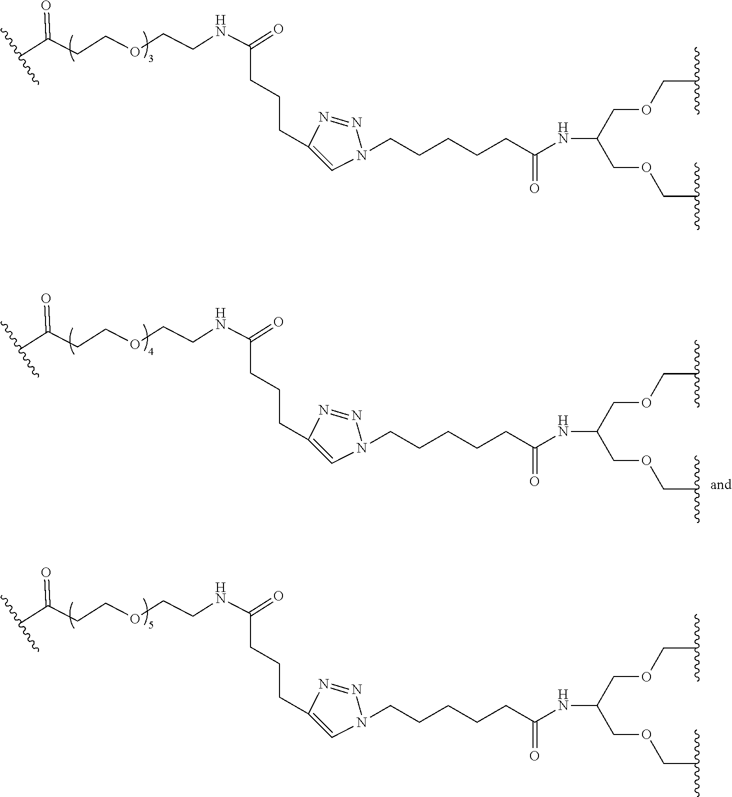

In certain embodiments, in the compound of Formula II, the Linker^{C} is selected from:

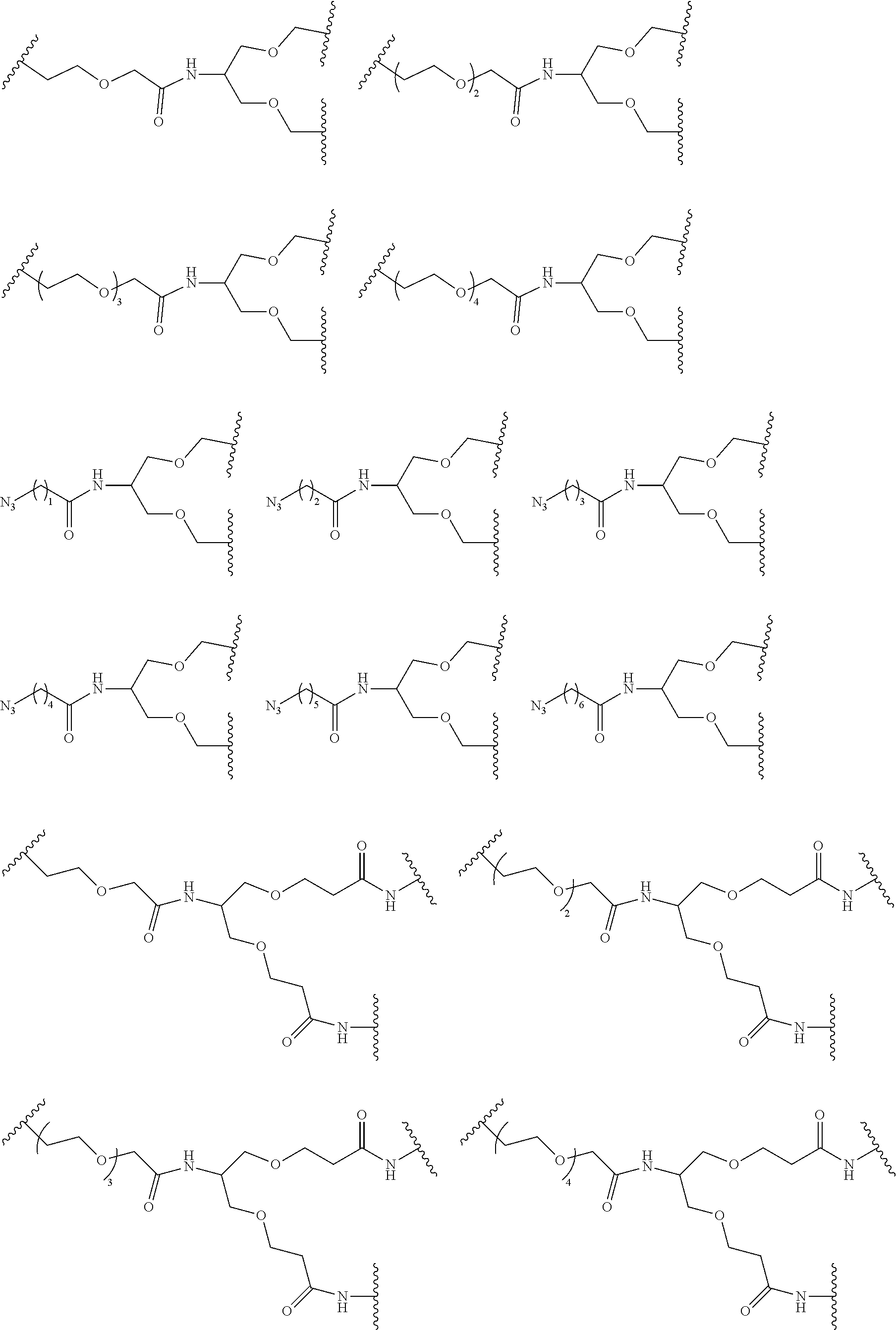

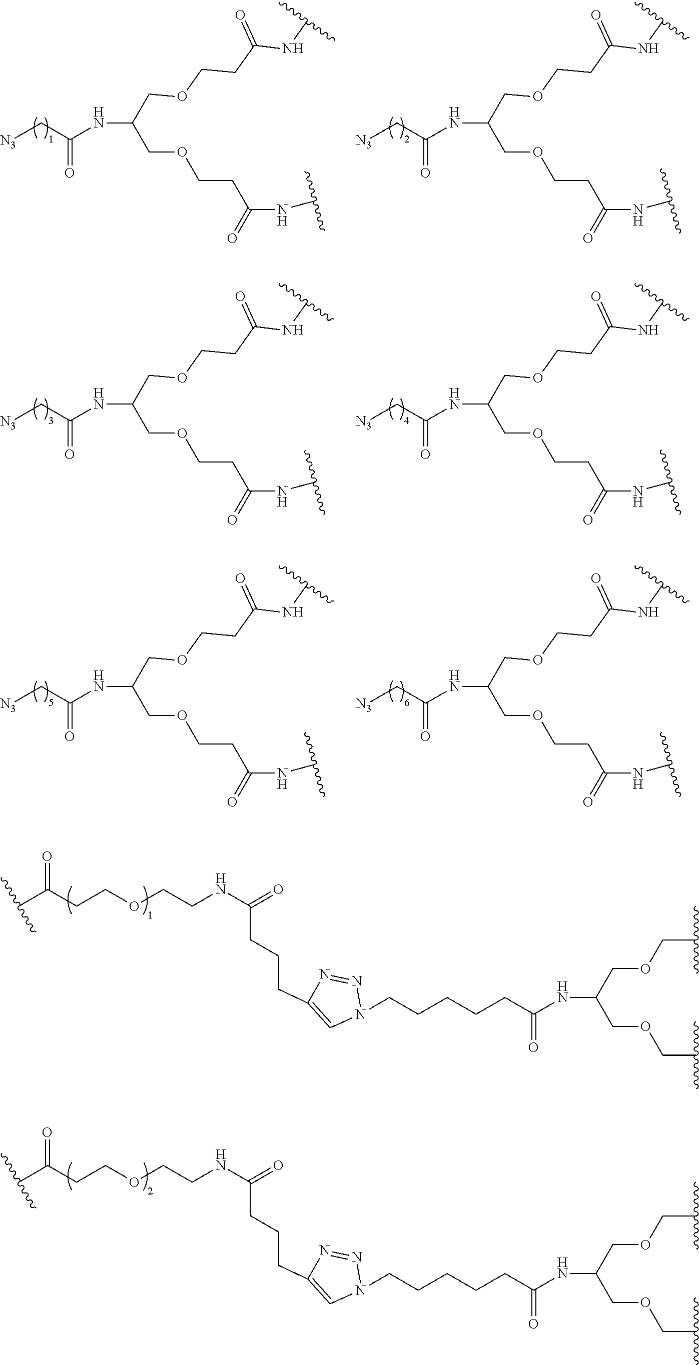

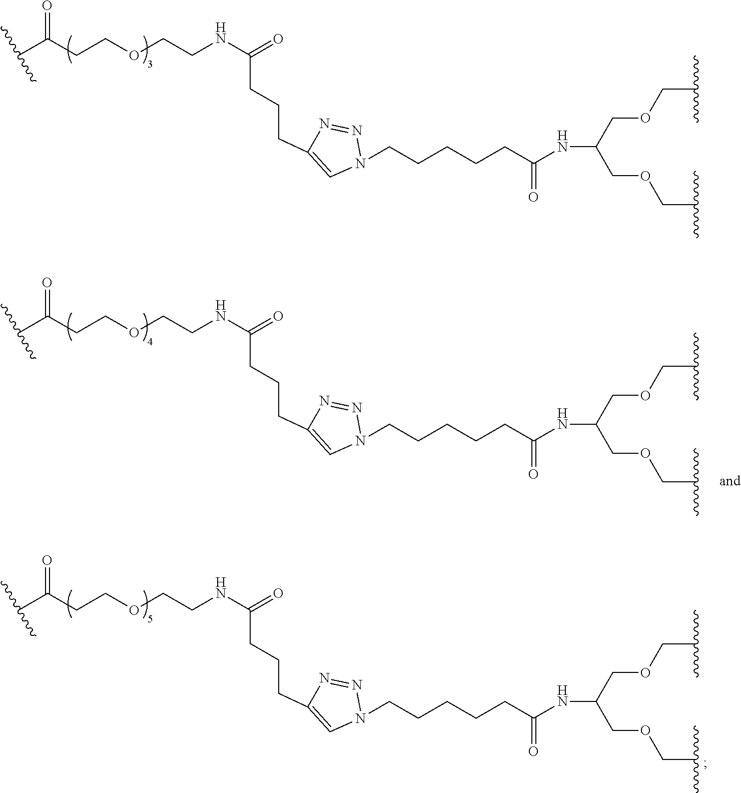

In certain embodiments, in the compound of Formula II, the Linker^{C} is selected from: wherein each is optionally substituted with 1, 2, 3, or 4 substituents substituent selected from R²¹.

In certain embodiments, in the compound of Formula II, the Linker^{C} is selected from;

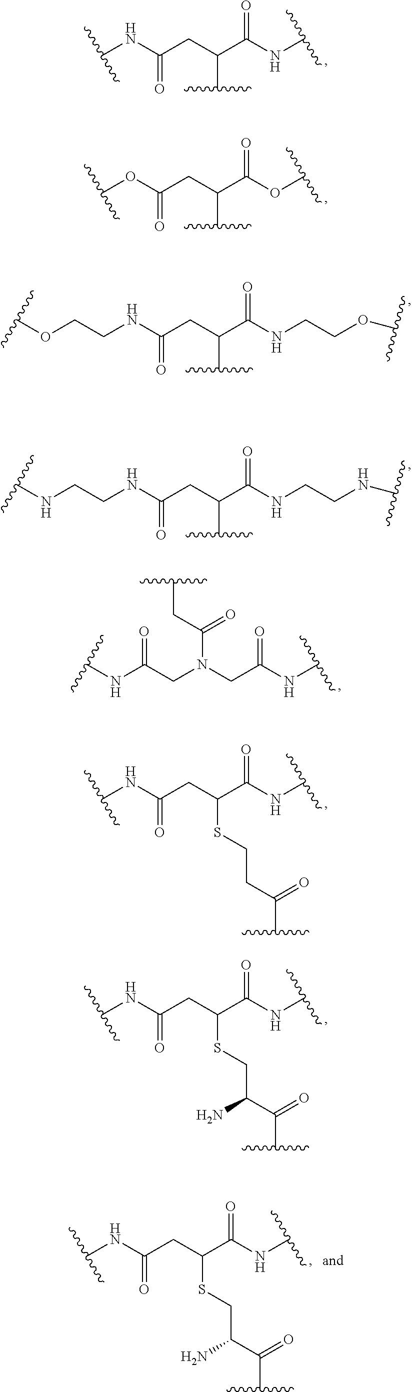

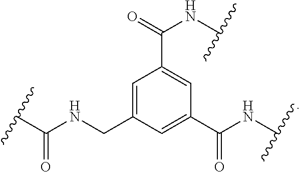 [SMILES: *NC(=O)c1cc(CNC(*)=O)cc(C(=O)N*)c1]

In certain embodiments, in the compound of Formula II, the Linker^{C} is selected from:

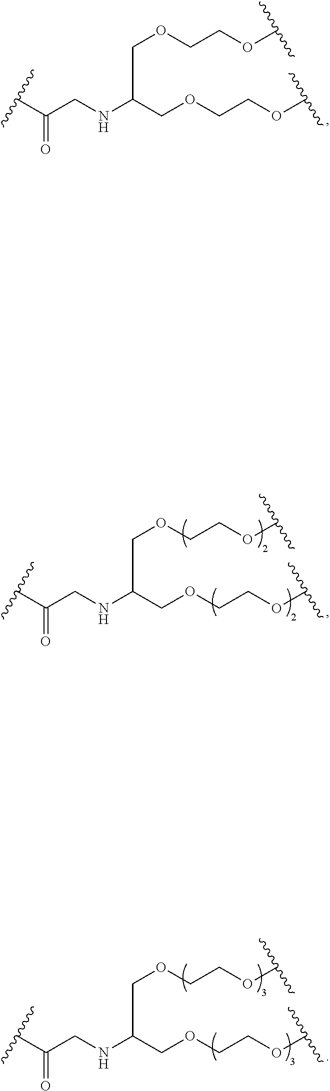

In certain embodiments, in the compound of Formula II, the Linker^{C} is selected from:

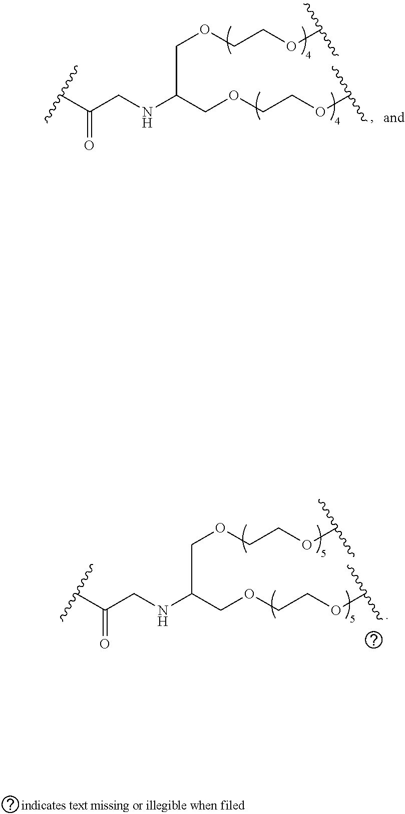

In certain embodiments, in the compound of Formula II, the Linker^{C} is selected from:

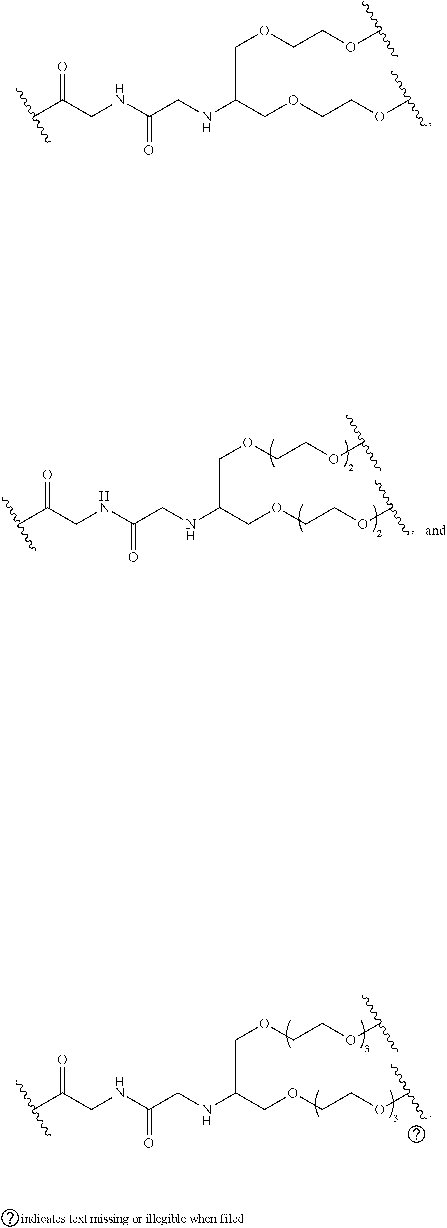

In certain embodiments, in the compound of Formula II, the Linker^{C} is selected from:

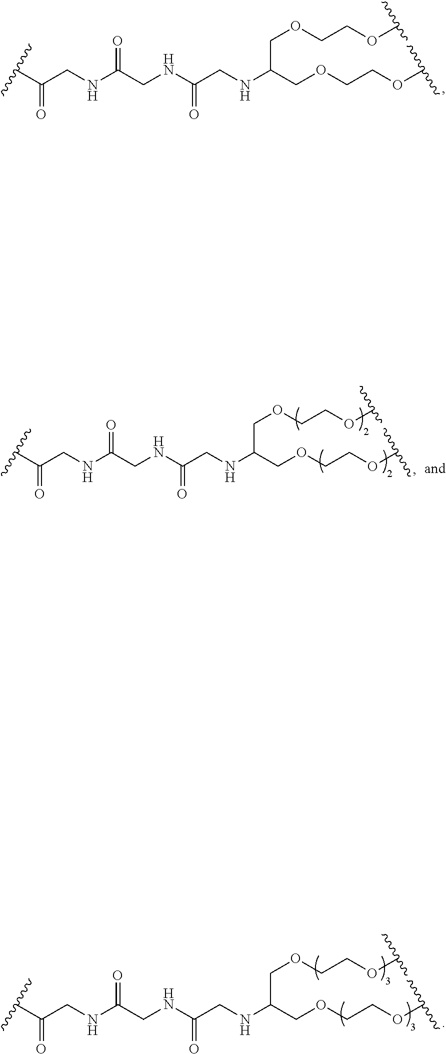

In certain embodiments, in the compound of Formula II, the Linker^{C} is selected from:

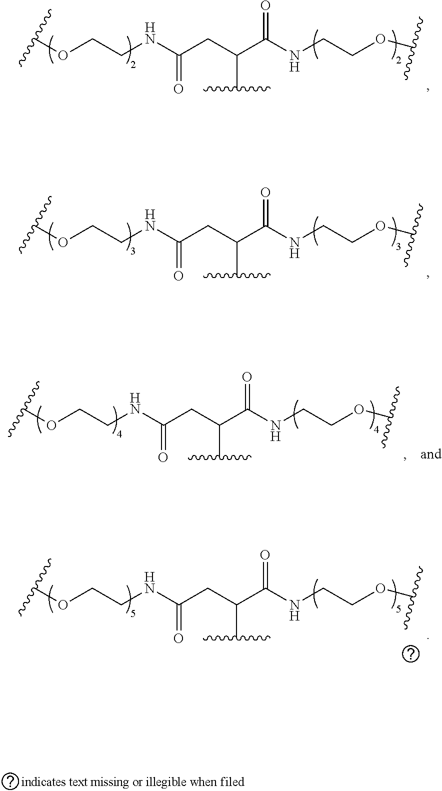

In certain embodiments, in the compound of Formula II, the Linker^{C} is selected from:

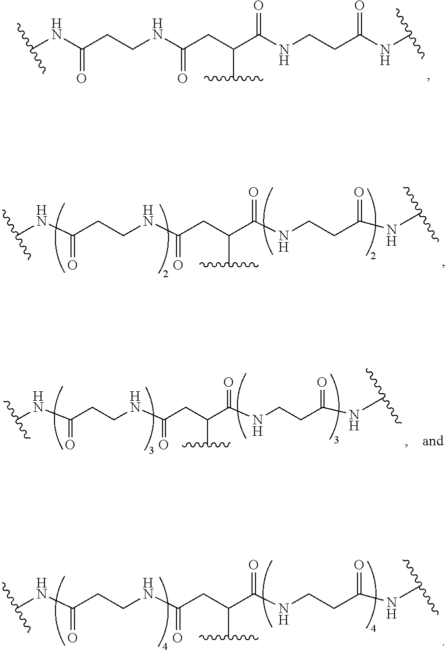

In certain embodiments, in the compound of Formula II, the Linker^{C} is selected from:

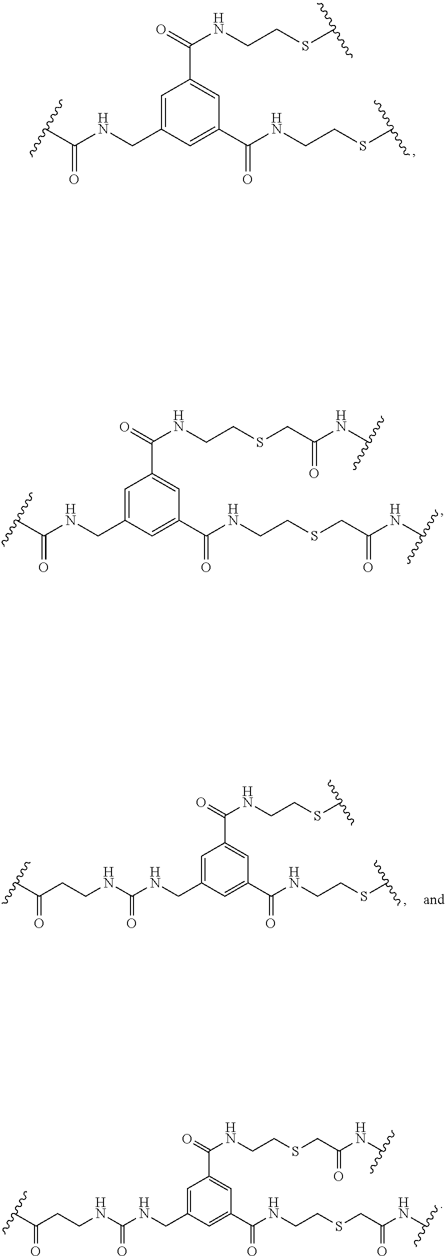 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCSCC(=O)N*)c1]

In certain embodiments, in the compound of Formula II, the Linker^{C}-(Linker^{A})₂ is selected from:

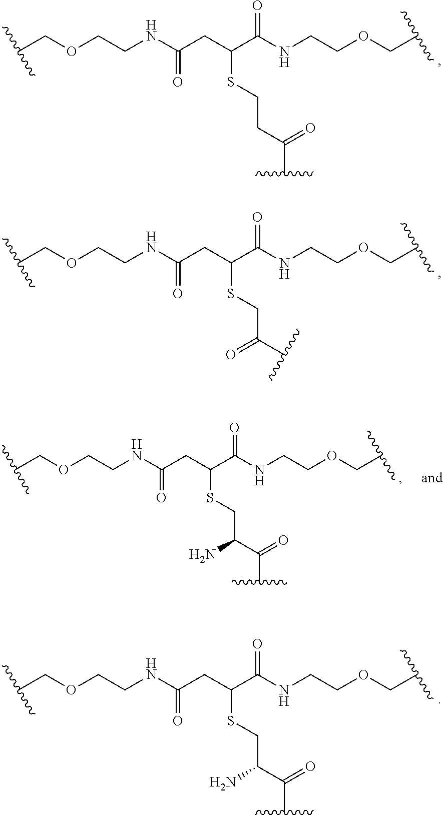 [SMILES: *COCCNC(=O)CC(SCCC(*)=O)C(=O)NCCOC* ; *COCCNC(=O)CC(SCC(*)=O)C(=O)NCCOC* ; *COCCNC(=O)CC(SC[C@H](N)C(*)=O)C(=O)NCCOC* ; *COCCNC(=O)CC(SC[C@@H](N)C(*)=O)C(=O)NCCOC*]

In certain embodiments, in the compound of Formula II, Linker^{C}-(Linker^{A})₂ is selected from:

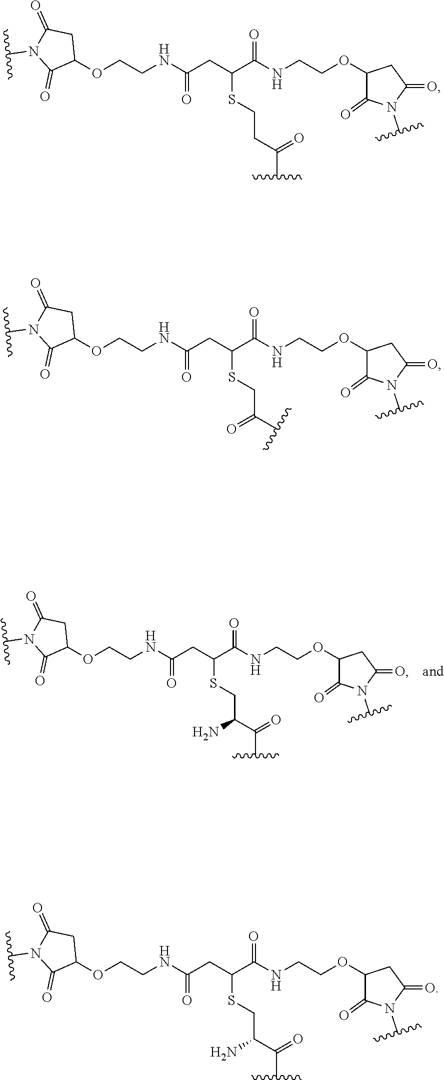 [SMILES: *C(=O)CCSC(CC(=O)NCCOC1CC(=O)N(*)C1=O)C(=O)NCCOC1CC(=O)N(*)C1=O ; *C(=O)CSC(CC(=O)NCCOC1CC(=O)N(*)C1=O)C(=O)NCCOC1CC(=O)N(*)C1=O ; *C(=O)[C@@H](N)CSC(CC(=O)NCCOC1CC(=O)N(*)C1=O)C(=O)NCCOC1CC(=O)N(*)C1=O ; *C(=O)[C@H](N)CSC(CC(=O)NCCOC1CC(=O)N(*)C1=O)C(=O)NCCOC1CC(=O)N(*)C1=O]

In certain embodiments, in the compound of Formula II, Linker^{C}-(Linker^{A})₂ is selected from:

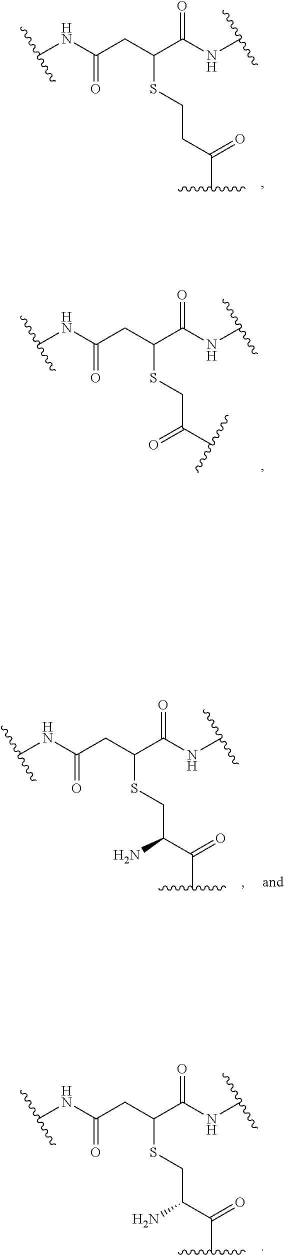 [SMILES: *NC(=O)CC(SCCC(*)=O)C(=O)N* ; *NC(=O)CC(SCC(*)=O)C(=O)N* ; *NC(=O)CC(SC[C@H](N)C(*)=O)C(=O)N* ; *NC(=O)CC(SC[C@@H](N)C(*)=O)C(=O)N*]

In certain embodiments, in the compound of Formula II, Linker^{C}-(Linker^{A})₂ is selected from:

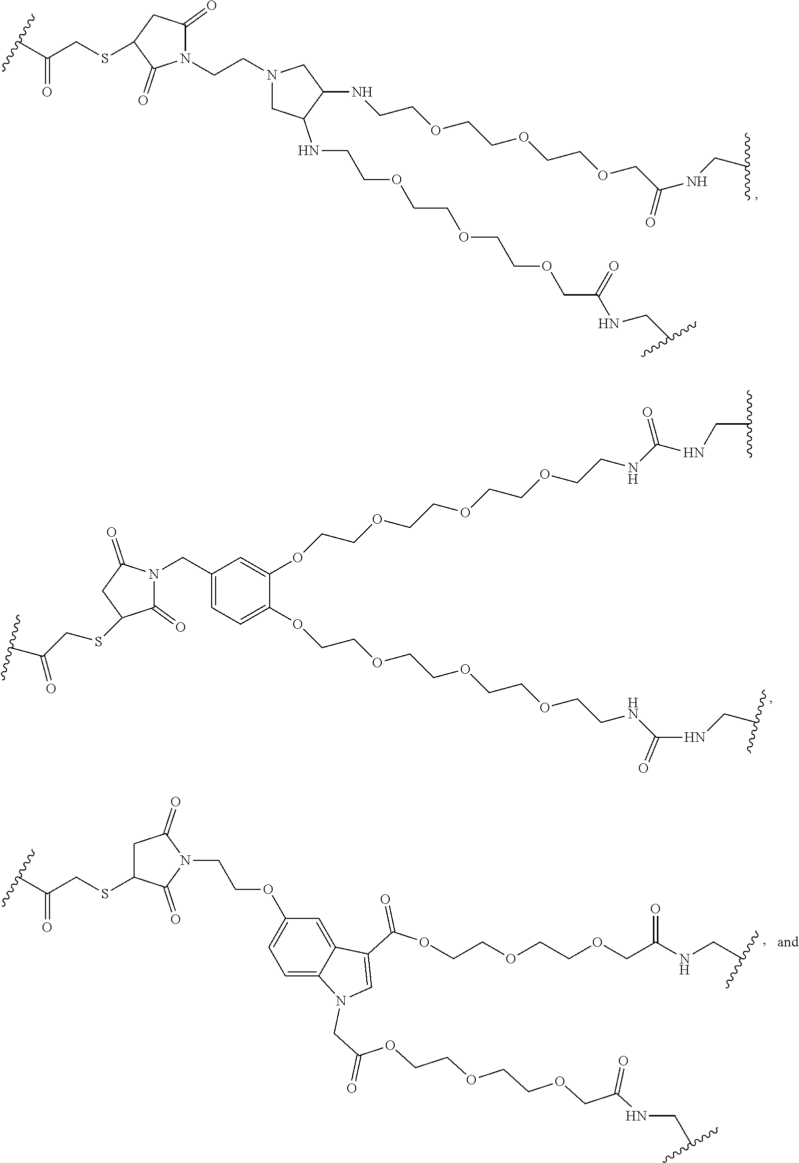 [SMILES: *CNC(=O)COCCOCCOCCNC1CN(CCN2C(=O)CC(SCC(*)=O)C2=O)CC1NCCOCCOCCOCC(=O)NC* ; *CNC(=O)NCCOCCOCCOCCOc1ccc(CN2C(=O)CC(SCC(*)=O)C2=O)cc1OCCOCCOCCOCCNC(=O)NC* ; *CNC(=O)COCCOCCOC(=O)Cn1cc(C(=O)OCCOCCOCC(=O)NC*)c2cc(OCCN3C(=O)CC(SCC(*)=O)C3=O)ccc21]

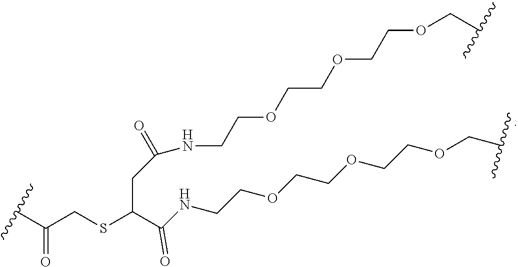 [SMILES: *COCCOCCOCCNC(=O)CC(SCC(*)=O)C(=O)NCCOCCOCCOC*]

In certain embodiments, in the compound of Formula II, Linker^{D} is selected from:

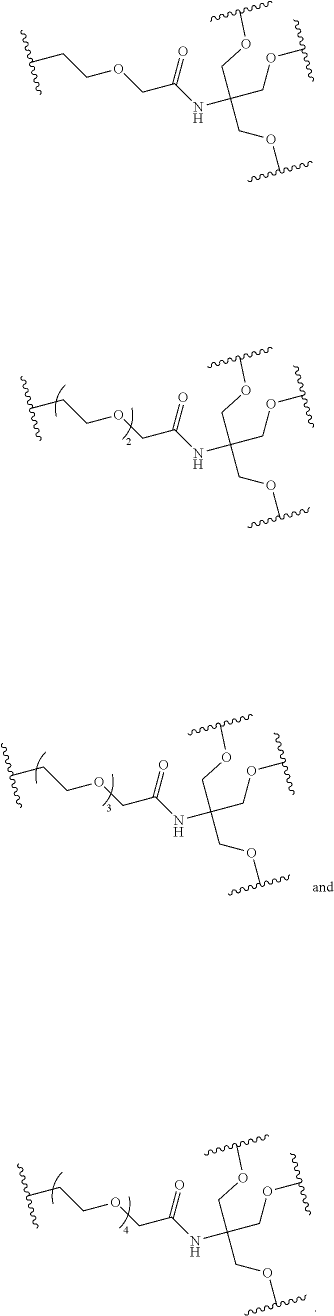

In certain embodiments, in the compound of Formula II, Linker^{D} is selected from:

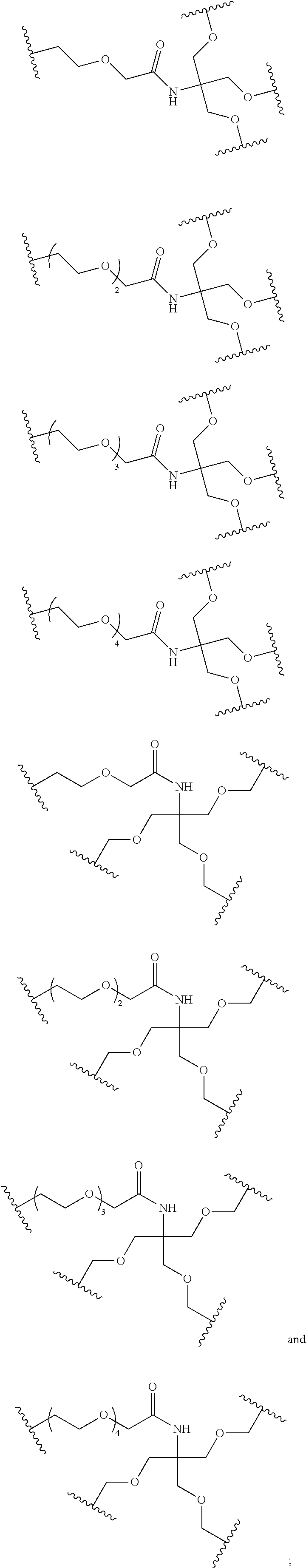

wherein each is optionally substituted with 1, 2, 3, or 4 substituents are selected from R²¹.

In certain embodiments, in the compound of Formula II, Linker^{B}-(Linker^{A}) is selected from

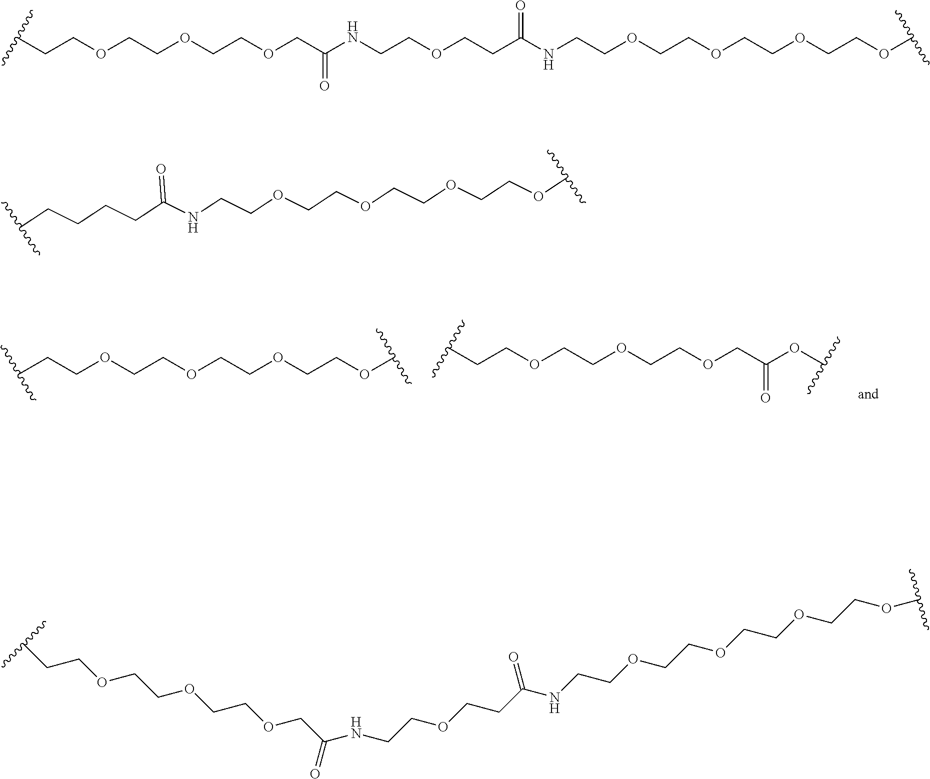 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, in the compound of Formula II, Linker^{C}-(Linker^{A}) is selected from

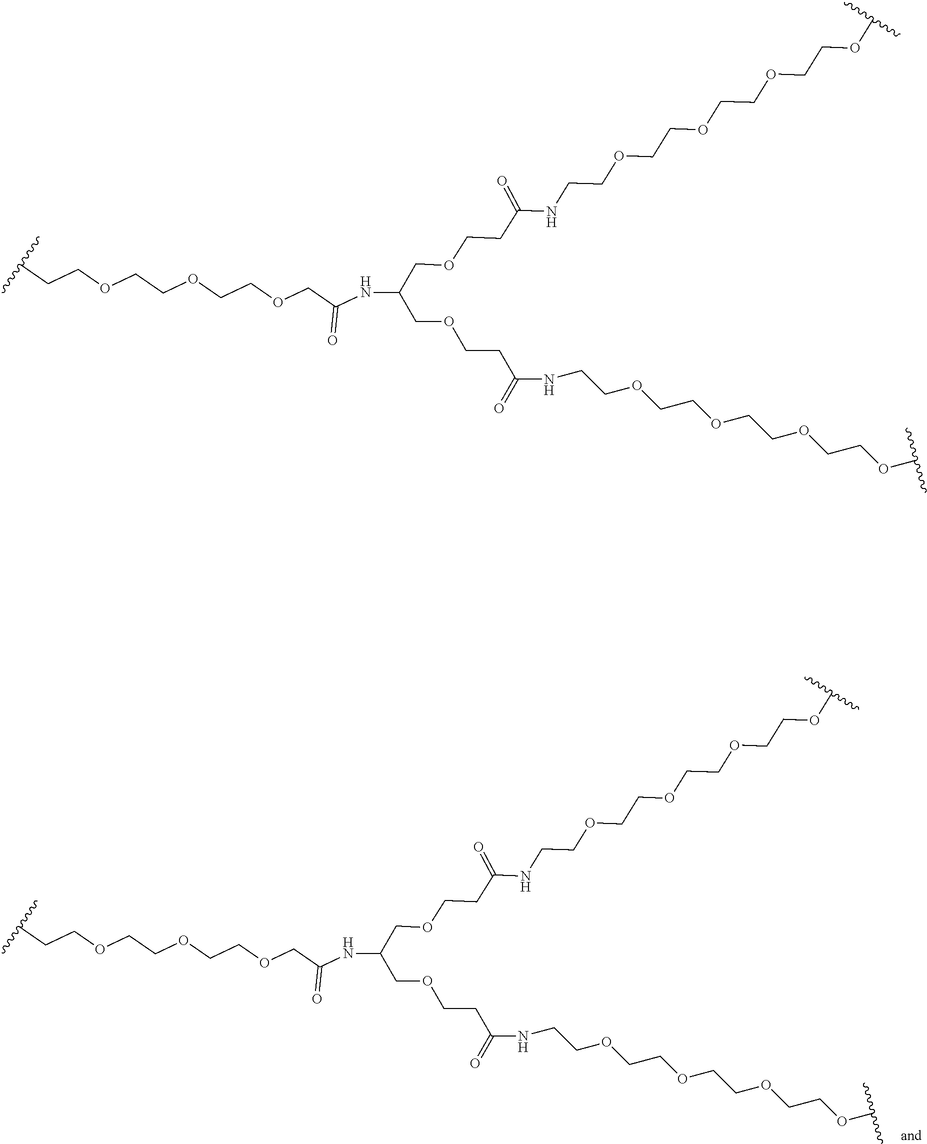 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO*]

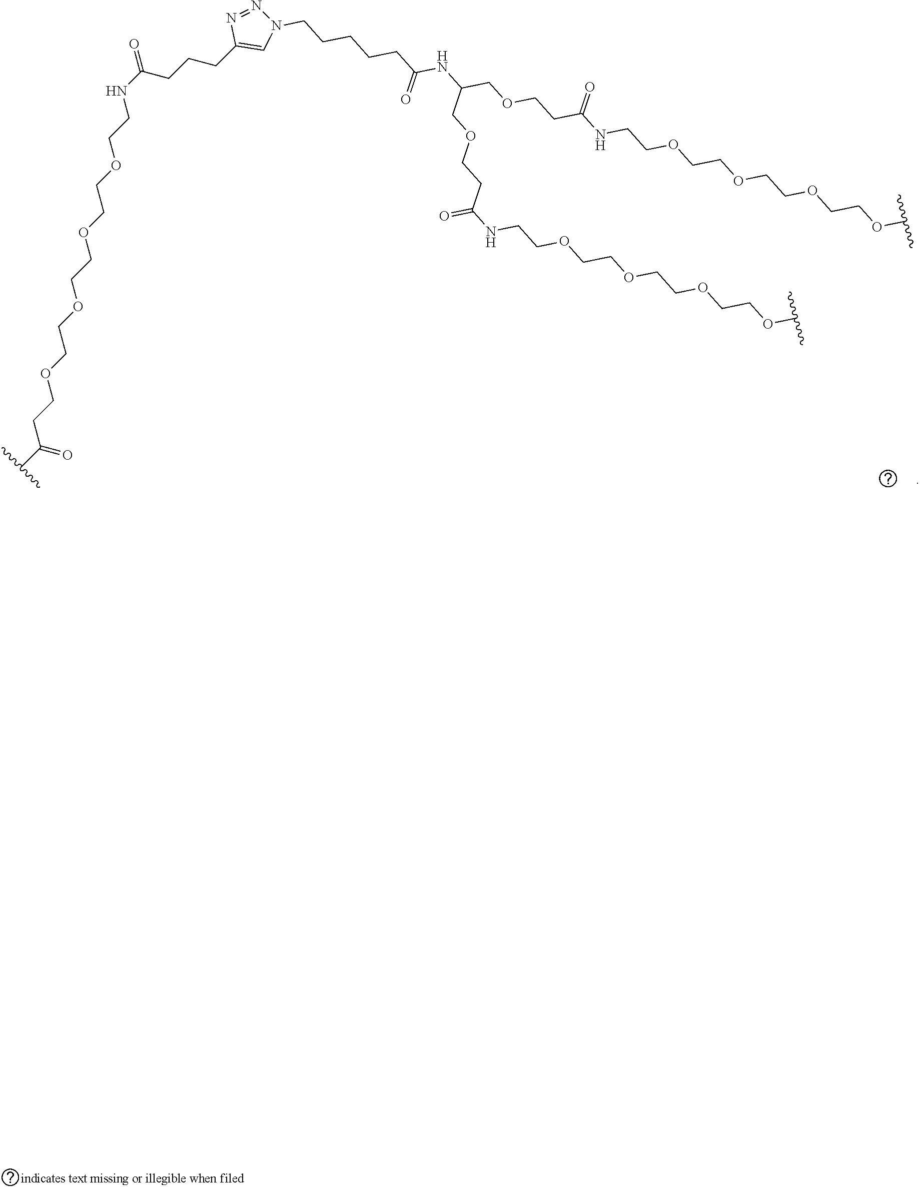 [SMILES: *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, in the compound of Formula II, Linker^{D}-(Linker^{A})₂ is selected from

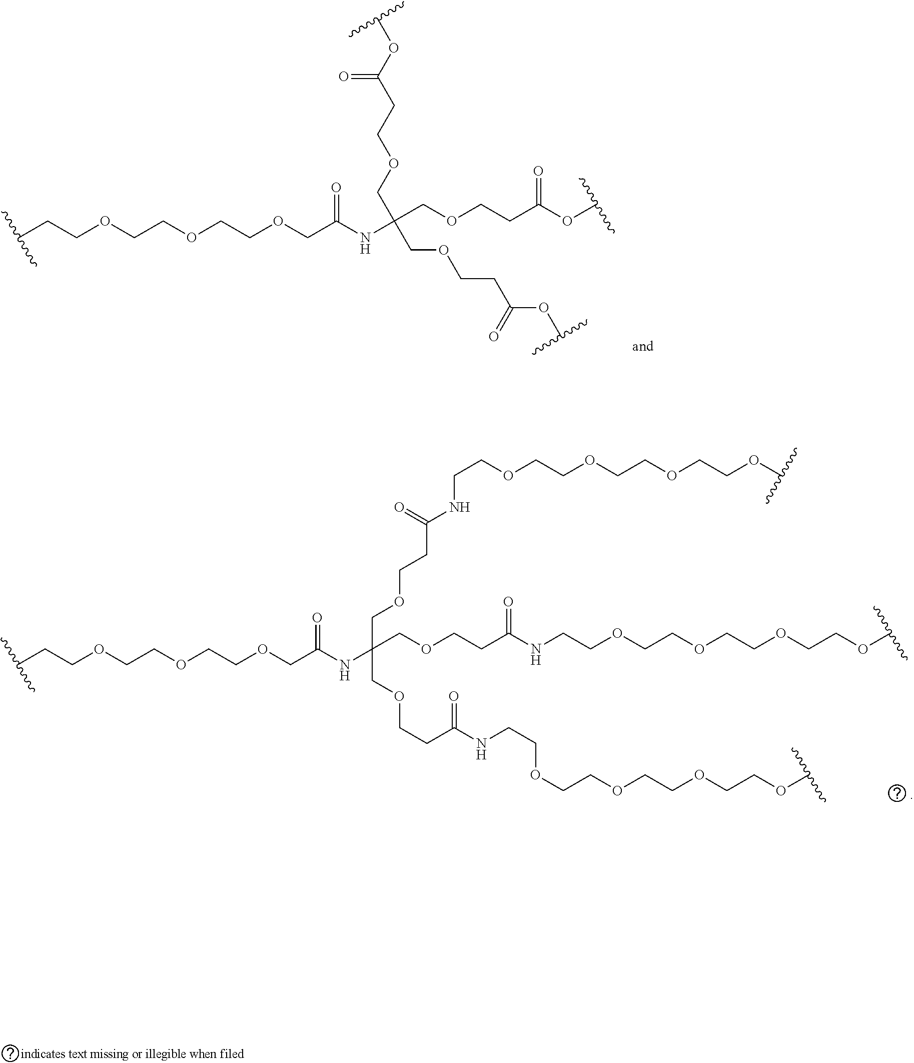 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

In various embodiments, R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷, C(O)R′, S(O)R³, C(S)R′, and S(O)₂R³.

In various embodiments, in the compound of Formula II, R⁵ is independently selected from hydrogen, heteroalkyl,

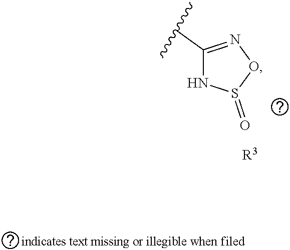

C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R′, C₀-C₆alkyl-S(O)R³, C₀-C₆alkyl-C(S)R³, C₀-C₆alkyl-S(O)₂R′, C₀-C₆alkyl-N(R⁸)—C(O)R³, C₀-C₆alkyl-N(R¹)—S(O)R³, C₀-C₆alkyl-N(R⁸)—C(S)R³, C₀-C₆alkyl-N(R⁸)—S(O)₂R³C₀-C₆alkyl-O—C(O)R′, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents.

In various embodiments, in the compound of Formula II, R⁶ and R⁷ are independently selected at each occurrence from hydrogen, heteroalkyl, alkyl, arylalkyl, heteroaryl alkyl, alkenyl, alkynyl, and, haloalkyl, heteroaryl, heterocycle, -alkyl-OR⁸, -alkyl-NR⁸R⁹, C(O)R³, S(O)R³, C(S)R³, and S(O)₂R³.

In various embodiments, in the compound of Formula II, R⁸ and R⁹ are independently selected at each occurrence from hydrogen, heteroalkyl, alkyl, arylalkyl, heteroarylalkyl, alkenyl, alkynyl, aryl, heteroaryl, and heterocycle.

In various embodiments, the compound of Formula II has the structure of Formula II-A. In various embodiments, in the compound of Formula IL, [Protein binder], [TNF binder] and [AATM] are as defined herein.

A compound of Formula II-A, having the structure:

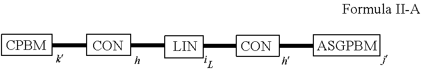

wherein:
[CPBM] is a cellular protein binding moiety selected from a [Protein binder], a [TNF binder], and a molecule that binds to an autoantibody [AATM];
[ASGPBM] is an asialoglycoprotein receptor binding moiety having the structure selected from

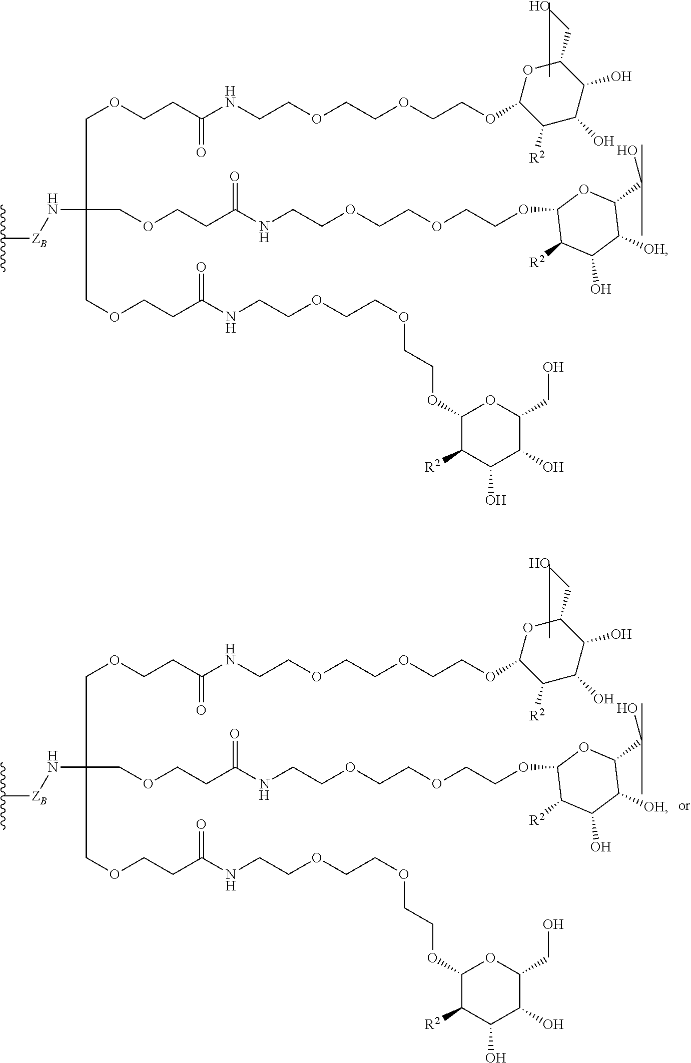

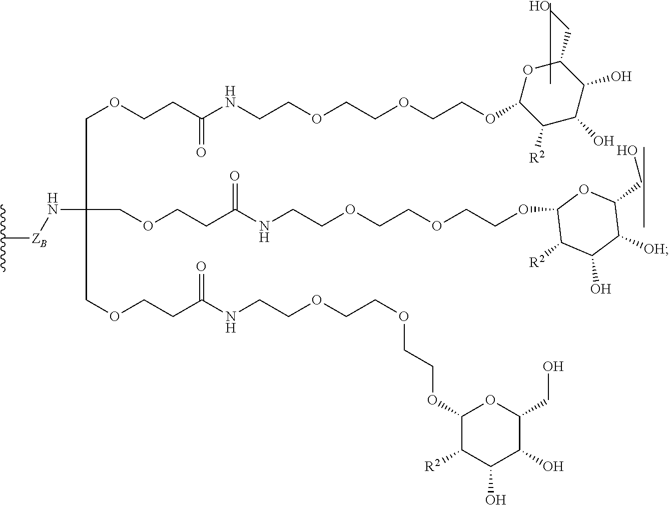

each [CON] is an optional connector chemical moiety which, when present, connects the [LIN] to [CPBM] or to [ASGPBM];
[LIN] is [LINKER] or [LINKER-2], each of which is a chemical moiety having a valency from 1 to 15, which covalently attaches to one or more [ASGPBM] or [CPBM] groups, optionally through a [CON], wherein the [LIN] optionally itself contains one or more [CON] groups;
   Z_{B} is absent, (CH₂)_{IM}, C(O)—(CH₂)_{IM}-, or C(O)—(CH₂)_{IM}—NR_{M},
   R_{M} is H or a C₁—C; alkyl group optionally substituted with one or two hydroxyl groups;
R² is

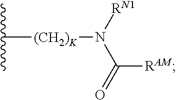

wherein R^{AM} is H, C₁-C₄ alkyl optionally substituted with up to 3 halo groups and one or two hydroxyl groups, —(CH₂)_{K}COOH, —(CH₂)_{K}C(O)O—(C₁-C₄ alkyl) optionally substituted with 1-3 halo groups, —O—C(O)—(C₁-C₄ alkyl) optionally substituted with 1-3 halo groups, —C(O)—(C₁-C₄ alkyl) optionally substituted with 1-3 halo groups, or —(CH₂)_{K}—NR^{N3}R^{N4}, or
R² is

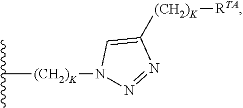

wherein
   R^{TA} is H, CN, NR^{N1}R^{N2}, —(CH₂)_{K}OH, —(CH₂)_{K}O(C₁-C₄ alkyl) optionally substituted with 1-3 halo groups, C₁-C₄ alkyl optionally substituted with 1-3 halo groups, —(CH₂)_{K}COOH, —(CH₂)_{K}C(O)O—(C₁-C₄ alkyl) optionally substituted with 1-3 halo groups, —O—C(O)—(C₁-C₄ alkyl) optionally substituted with 1-3 halo groups, or —C(O)—(C₁-C₄ alkyl) optionally substituted with 1-3 halo groups, or
   R^{TA} is a C₃-C₁₀ aryl or a three- to ten-membered heteroaryl group containing up to 5 heteroaryl atoms, each of the aryl or heteroaryl groups being optionally substituted with up to three CN, NR^{N1}R^{N2}, —(CH₂)_{K}OH, —(CH₂)_{K}O(C₁-C₄ alkyl) optionally substituted with 1-3 halo groups, C₁-C₃ alkyl optionally substituted with 1-3 halo groups or 1-2 hydroxy groups, —O—(C₁-C₃-alkyl) optionally substituted from 1-3 halo groups, —(CH₂)_{K}COOH, —(CH₂)_{K}C(O)O—(C₁-C₄ alkyl) optionally substituted with 1-3 halo groups, O—C(O)—(C₁-C₄ alkyl) optionally substituted with 1-3 halo groups, or —(CH₂)_{K}C(O)—(C₁-C₄ alkyl) optionally substituted with 1-3 halo groups, or
   R^{TA} is

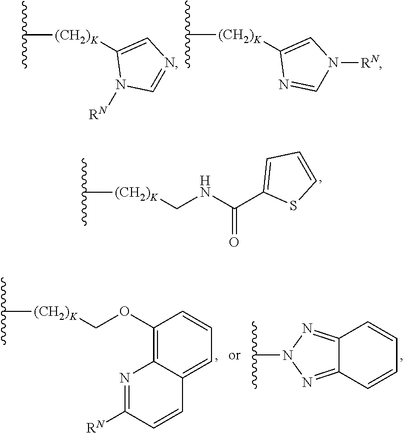

optionally substituted with up to three C₁-C₃ alkyl groups which are optionally substituted with up to three halo groups; or
R^{TA} is

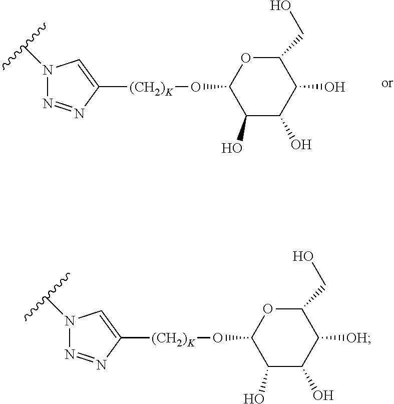

R^{N}, R^{N1}, R^{N2}, R^{N3}, R^{N4} are each independently H or C₁-C₃ alkyl optionally substituted with one to three halo groups or one or two hydroxyl groups and each —(CH₂)x group is optionally substituted with 1-4 C₁-C₃ alkyl groups which are optionally substituted with 1-3 fluoro groups or 1-2 hydroxyl groups;
IM is independently at each occurrence an integer from 0 to 6;
K is independently at each occurrence an integer from 0 to 4;
k′ is an integer ranging from 1 to 15;
j′ is an integer ranging from 1 to 15;
h and h′ are each independently an integer ranging from 0 to 15;
i_{L} is 0 to 15;
with the proviso that at least one of h, h′, and i_{L} is at least 1,
or a salt, stereoisomer, or solvate thereof.

In various embodiments, in the compound of Formula II-A, R₂ is —NC(═O)CH₃.

### D. Other-Based ASGPR-Binding Moieties

In some embodiments, the ASGPR binding moieties can be any of the moieties described in: Reshitko, G. S., et al., “Synthesis and Evaluation of New Trivalent Ligands for Hepatocyte Targeting via the Asialoglycoprotein Receptor,” Bioconjugate Chem, doi: 10.1021/acs.bioconjchem.0c00202; Majouga, A. G., et al., “Identification of Novel Small-Molecule ASGP-R Ligands,” Current Drug Delivery, 2016, 13, 1303-1312, doi: 10.2174/1567201813666160719144651; Olshanova, A. S., et al., “Synthesis of a new betulinic acid glycoconjugate with N-acetyl-D-galactosamine for the targeted delivery to hepatocellular carcinoma cells,” Russian Chemical Bulletin, International Edition, Vol. 69, No. 1, pp. 158-163, January 2020; Yamansarov, E. Yu., et al., “New ASGPR-targeted ligands based on glycoconjugated natural triterpenoids,” Russian Chemical Bulletin, International Edition, Vol. 68, No. 12, pp. 2331-2338, December 2019; Congdon, M. D., et al., “Enhanced Binding and Reduced Immunogenicity of Glycoconjugates Prepared via Solid-State Photoactivation of Aliphatic Diazirine Carbohydrates,” Bioconjugate Chem, doi. 10.1021/acs.bioconjchem.0c00555; and Dhawan, V., et al., “Polysaccharide conjugates surpass monosaccharide ligands in hepatospecific targeting—Synthesis and comparative in silico and in vitro assessment,” Carbohydrate Research 509 (2021) 108417, doi: 10.1016fj.carres.2021.108417. The following ASGPR binding moieties are illustrative and not intended to be limiting.

### 1. GalNAc-Tyrosine Based Moieties

In some embodiments, the ASGPR binding moiety can be a moiety having the structure of M1, M2, M3, or M4, or a combination thereof. In the structures of M1, M2, M3, and M4, X is independently at each occurrence O, NH, or S. In various embodiments, compounds of Formula I or Formula II can have one, two, or three ASGPR binding moieties with the structure of M1, M2, M3, or M4.

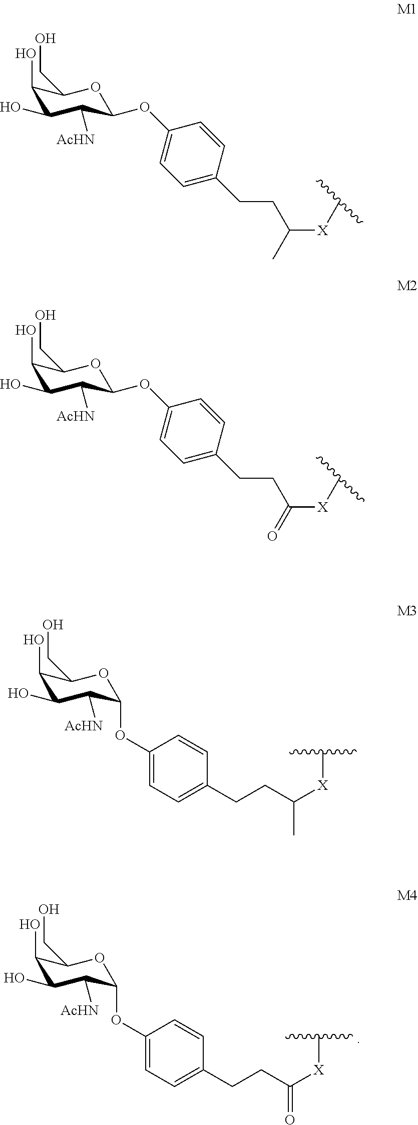

In various embodiments, ASGPR binding moieties M1 to M4 can be conjugated to any suitable [CON], [Linker], or [Linker-2] as described herein and in Congdon, M. D., et al., “Enhanced Binding and Reduced Immunogenicity of Glycoconjugates Prepared via Solid-State Photoactivation of Aliphatic Diazirine Carbohydrates,” Bioconjugate Chem, doi: 10.1021/acs.bioconjchem.0c00555.

### 2. Trivalent Triazole-Based Moieties

In some embodiments, the ASGPR binding moiety can be a moiety having the structure of M5:

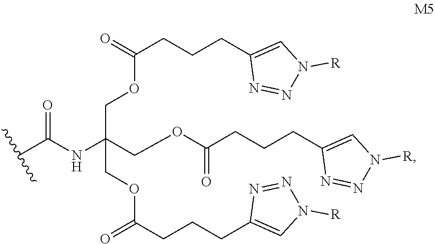

In the structures M5, each R is independently at each occurrence R₁ or R₂,

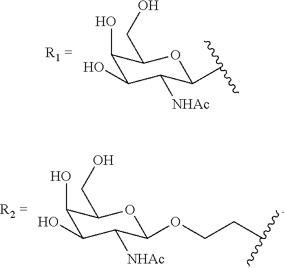 [SMILES: *C1OC(CO)C(O)C(O)C1NC(C)=O ; *CCOC1OC(CO)C(O)C(O)C1NC(C)=O]

In various embodiments, compounds of Formula I or Formula II contain an ASGPR binding moiety with the structure of M5. In various embodiments, each R in M5 is R₁. In various embodiments, each R in M5 is R₂.

In various embodiments, ASGPR binding moiety M5 can be conjugated/bonded to any suitable [CON], [Linker], or [Linker-2] as described herein and in Reshitko, G. S., et al., “Synthesis and Evaluation of New Trivalent Ligands for Hepatocyte Targeting via the Asialoglycoprotein Receptor,” Bioconjugate Chem, doi: 10.1021/acs.bioconjchem.0c00202.

### 3. Galactose- and Agarose-Derived Behenic Acid Ester Moieties

In various embodiments, the ASGPR binding moiety can be the galactose behenic acid ester-derived moiety M7:

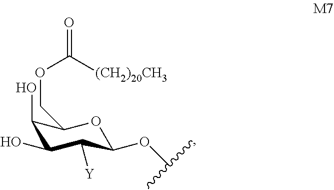

In the structure M7, Y is OH or NHAc.

In various embodiments, the ASGPR binding moiety can be the agarose behenic acid ester-derived moiety M8:

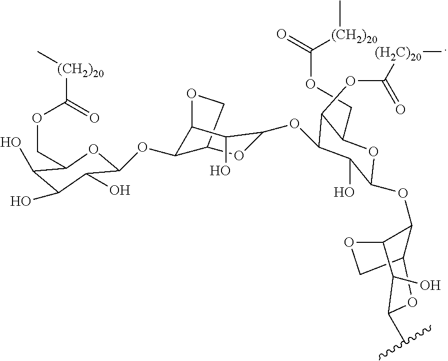

In various embodiments, ASGPR binding moieties M7 and M8 can be conjugated to any suitable [CON], [Linker], or [Linker-2] as described herein and in Dhawan, V., et al., “Polysaccharide conjugates surpass monosaccharide ligands in hepatospecific targeting—Synthesis and comparative in silico and in vitro assessment,” Carbohydrate Research 509 (2021) 108417, doi: 10.1016/j.carres.2021.108417.

### 4. Other Small Molecule ASGPR Binding Moieties

In various embodiments, the ASGPR binding moiety can be any of the compounds 2-18 below:

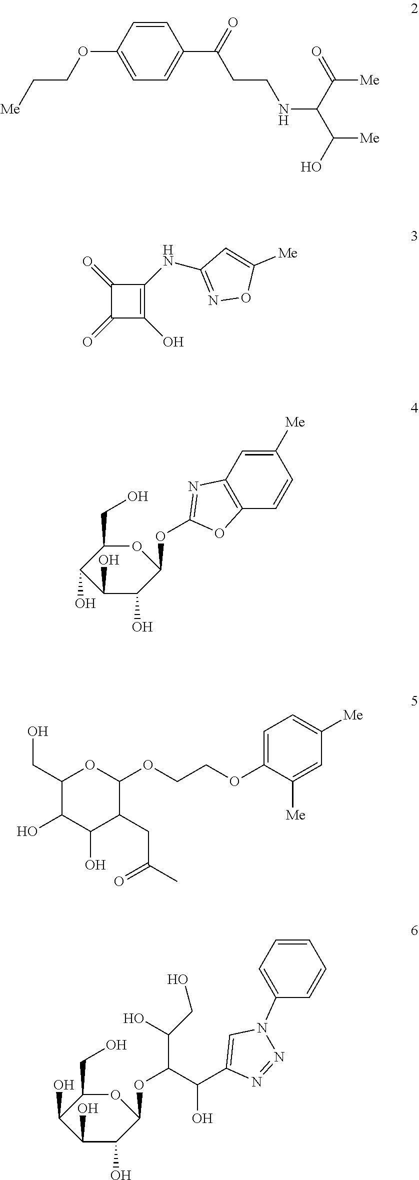

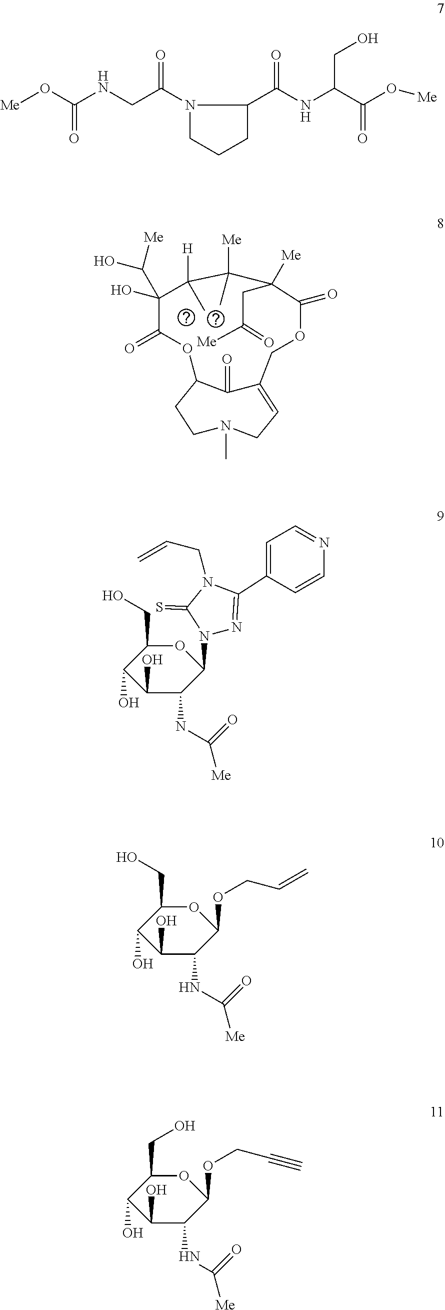

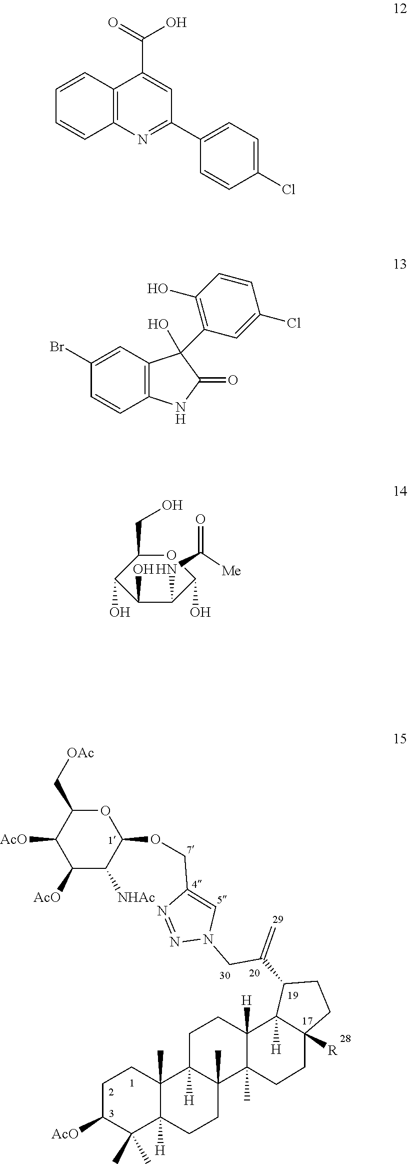

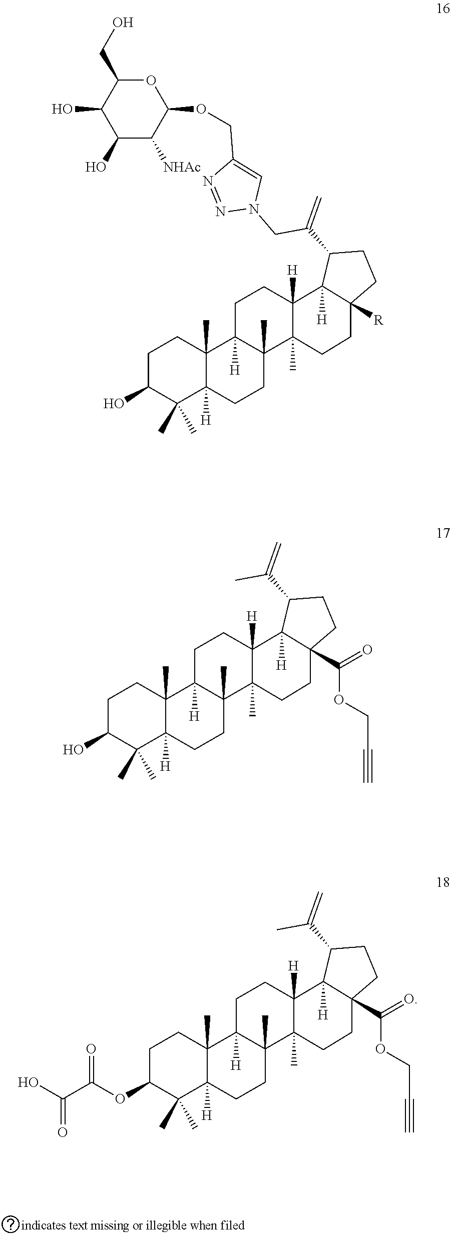

In various embodiments, in compounds 15 and 16, R is CH₂OAc, COOH, or CH₂OH. Compounds 2-18 can be conjugated/bonded to any suitable [CON], [Linker], or [Linker-2] as described herein and in Majouga, A. G., et al., “Identification of Novel Small-Molecule ASGP-R Ligands,” Current Drug Delivery, 2016, 13, 1303-1312, doi: 10.2174/1567201813666160719144651; Olshanova, A. S., et al., “Synthesis of a new betulinic acid glycoconjugate with N-acetyl-D-galactosamine for the targeted delivery to hepatocellular carcinoma cells,” Russian Chemical Bulletin, International Edition, Vol. 69, No. 1, pp. 158-163, January 2020; Yamansarov, E. Yu., et al., “New ASGPR-targeted ligands based on glycoconjugated natural triterpenoids,” Russian Chemical Bulletin, International Edition, Vol. 68, No. 12, pp. 2331-2338, December 2019. Compounds 2-18 can be attached through any suitable reactive group contained therein. Without limitation, compounds 2-13 can be attached to a CON], [Linker], or [Linker-2] through or by reaction with at least one OH, NH, vinyl, alkynyl, amide, acid, ester, ketone, or aromatic halogen contained in compounds 2-18. Suitable reaction modes for attaching compounds 2-18 to a [CON], [Linker], or [Linker-2] as described herein include, but are not limited to, substitution (e.g. alkylation of OH or NH groups), esterification (forming an ester), amidation (forming an amide), transesterification (exchanging one ester for another), transamidation (exchanging one amide for another), azide-alkyne cycloaddition, and other reactions capable of forming C—C, N—C, or O—C bonds with vinyl and alkynyl groups such as cycloadditions, aminations, oxidations, alkylations, rearrangement reactions (e.g. Claisen, Cope, etc.), and the like.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹³N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989): Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

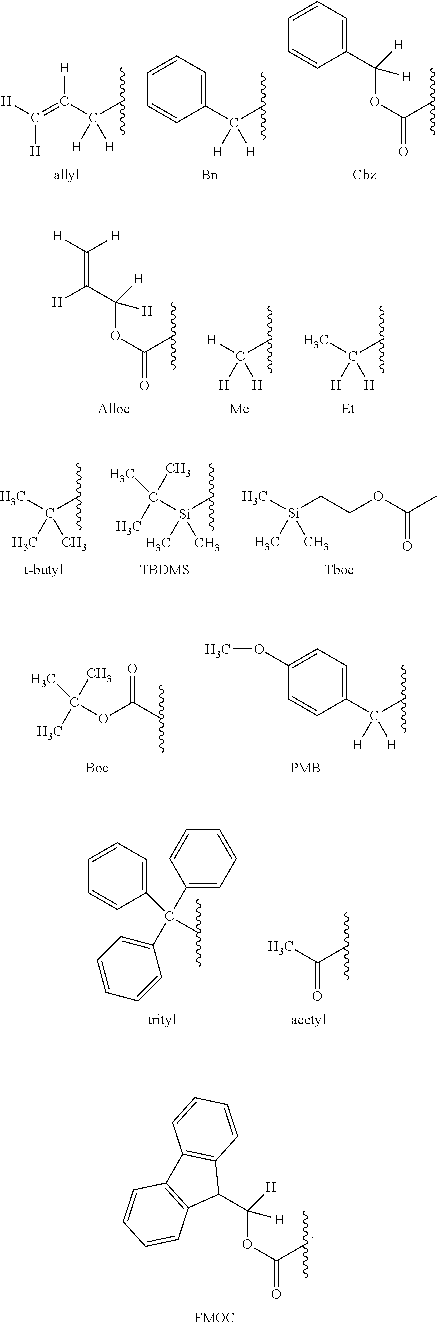

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans) buccal, (trans) urethral, vaginal (e.g., trans- and perivaginally), (intra) nasal and (trans) rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

The compounds of the disclosure can be used to treat certain diseases and/or disorders, such as, but not limited to, autoimmune diseases (such as but not limited to IgA nephropathy), cancer, inflammation, and any other disease or disorder contemplated herein.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats the disease or disorder.

In certain embodiments, administering the compound(s) described herein to the subject allows for administering a lower dose of the additional therapeutic agent as compared to the dose of the additional therapeutic agent alone that is required to achieve similar results in treating the disease or disorder in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating the disease or disorder, and/or with an additional therapeutic agents that reduce or ameliorate the symptoms and/or side-effects of therapeutic agent used in the treatment of the disease or disorder. These additional therapeutic agents may comprise compounds that are commercially available or synthetically accessible to those skilled in the art. When the additional therapeutic agents useful for treating the disease or disorder are used, these additional therapeutic agents are known to treat, or reduce the symptoms of the disease or disorder.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100/o, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the disclosed methods. The following working examples therefore, point out specific embodiments of the present disclosure, and are not to be construed as limiting in any way the remainder of the disclosure.

The materials and methods used in the experiments presented in this Experimental Example are now described.

### Example 1: Synthesis of Certain ASGPRBM Groups

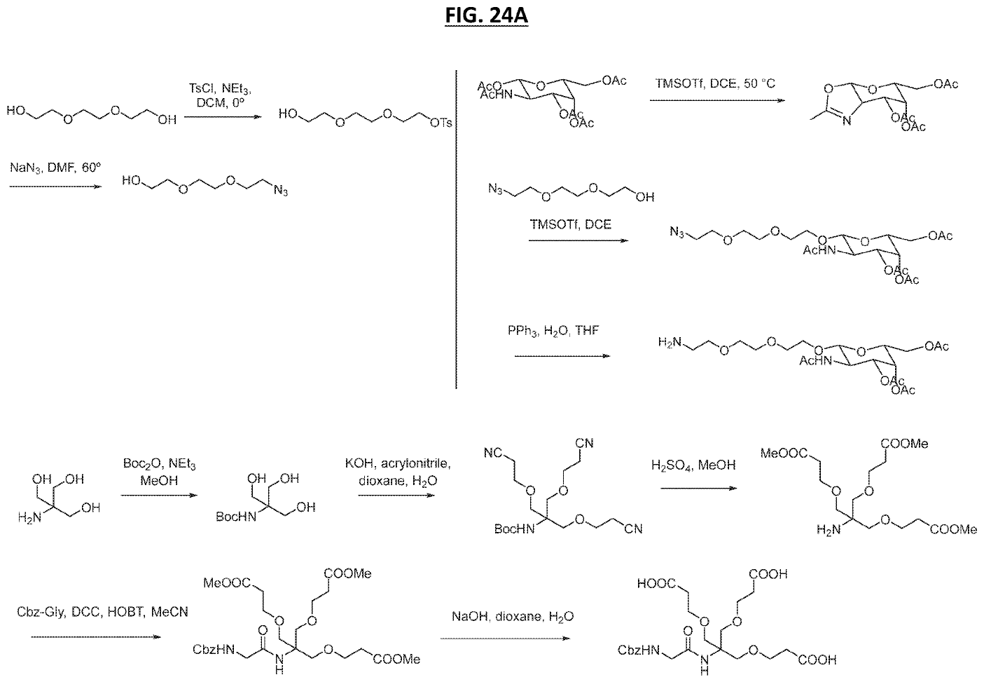

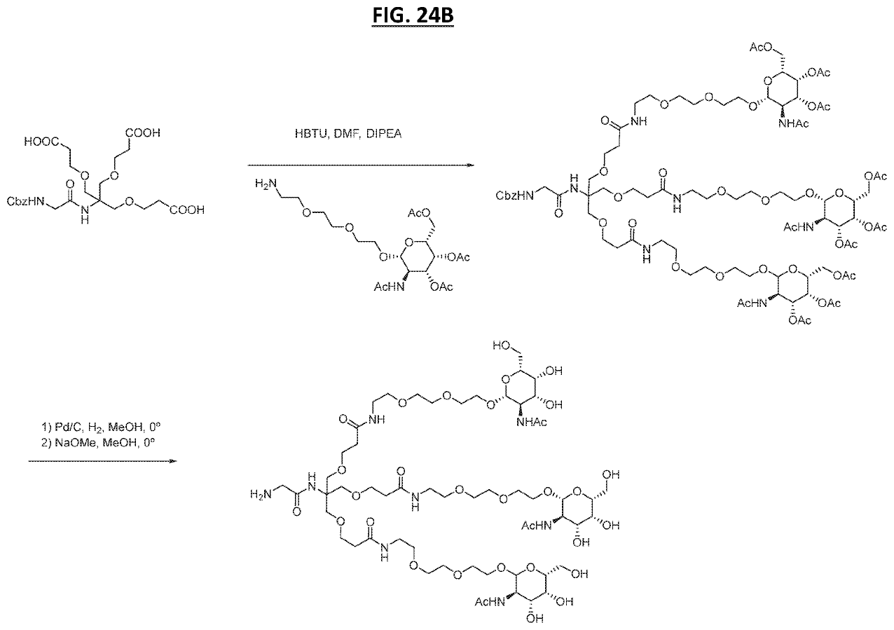

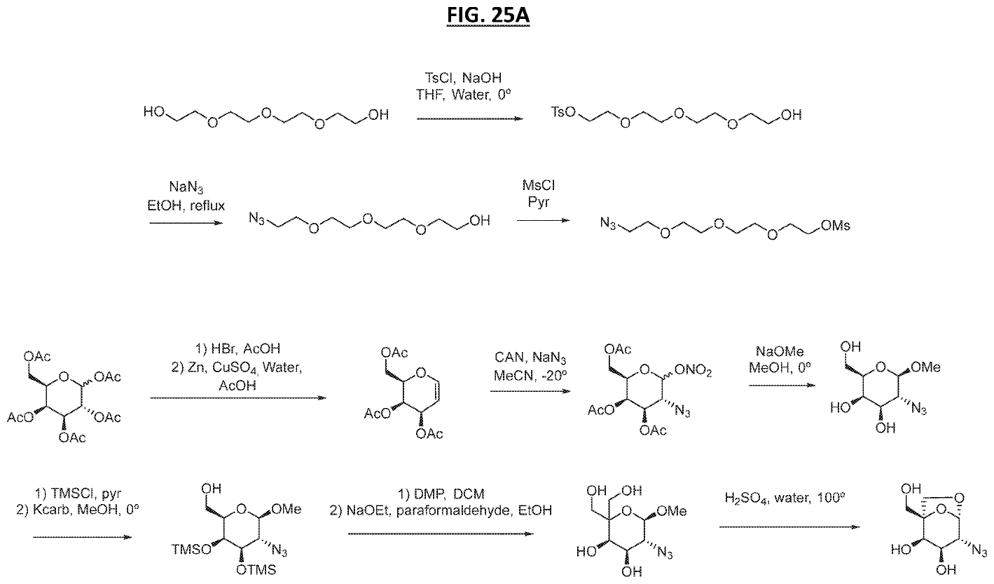

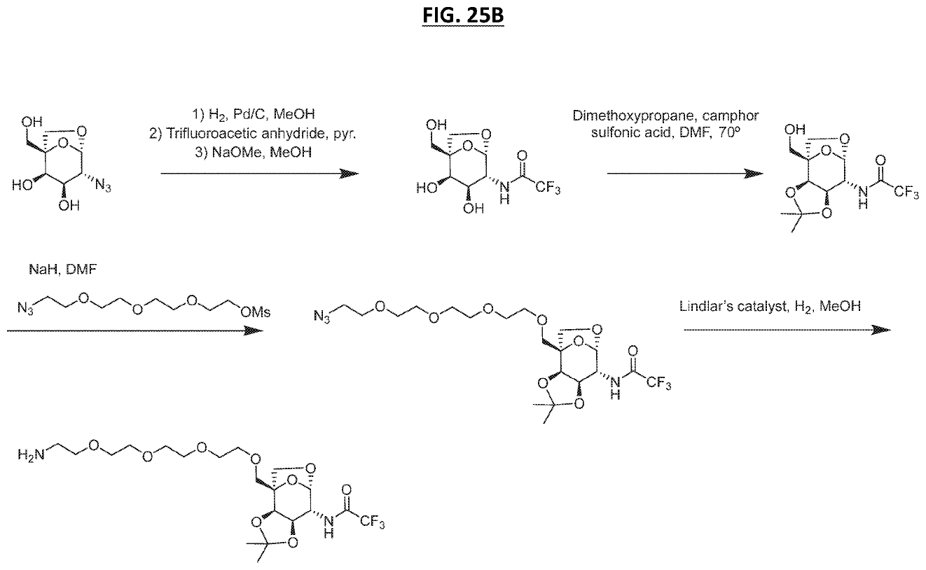

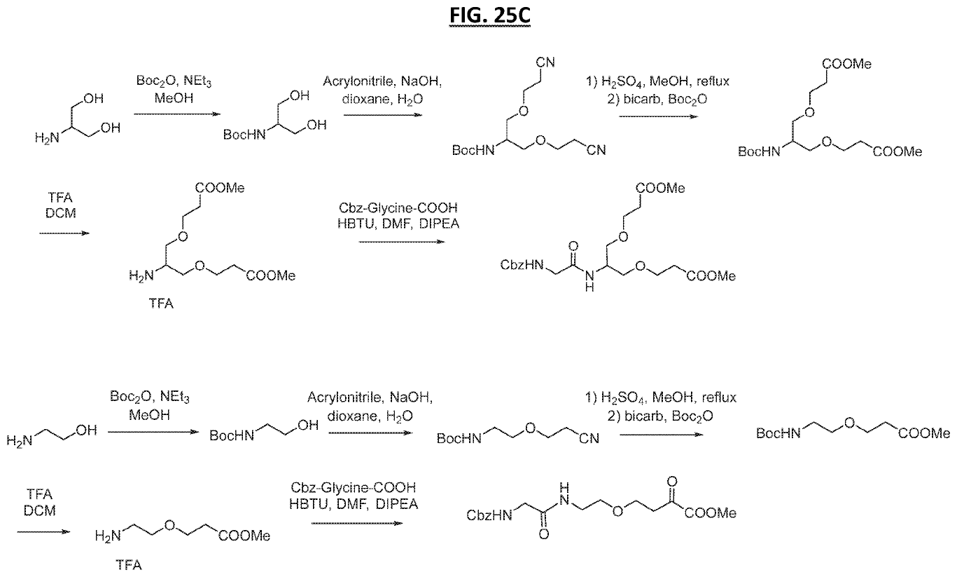

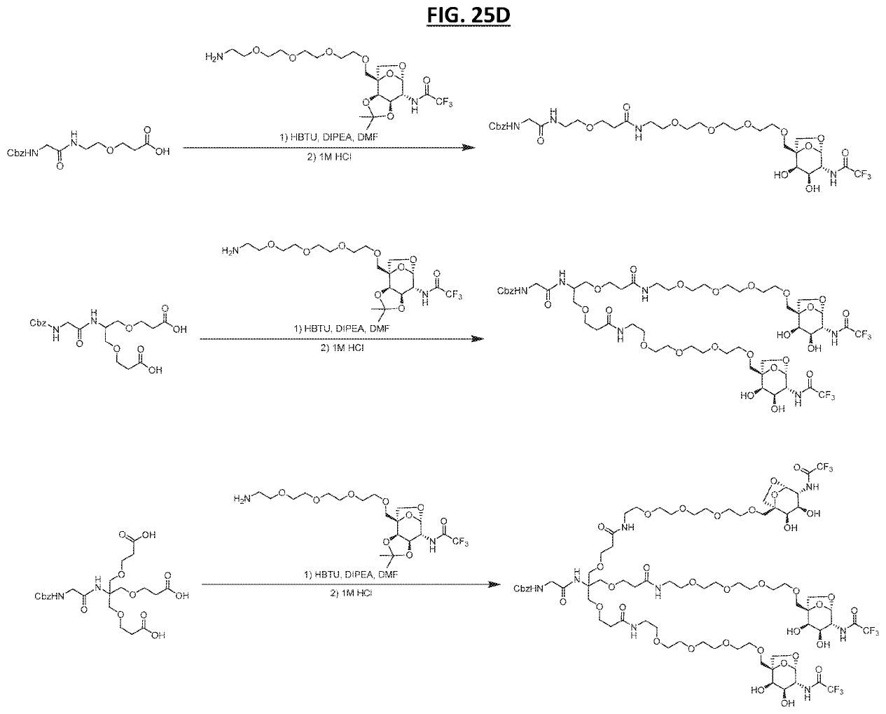

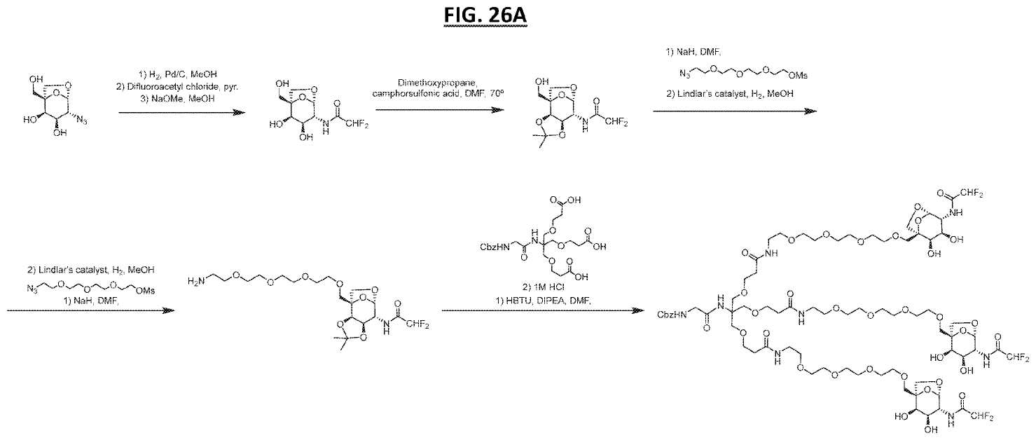

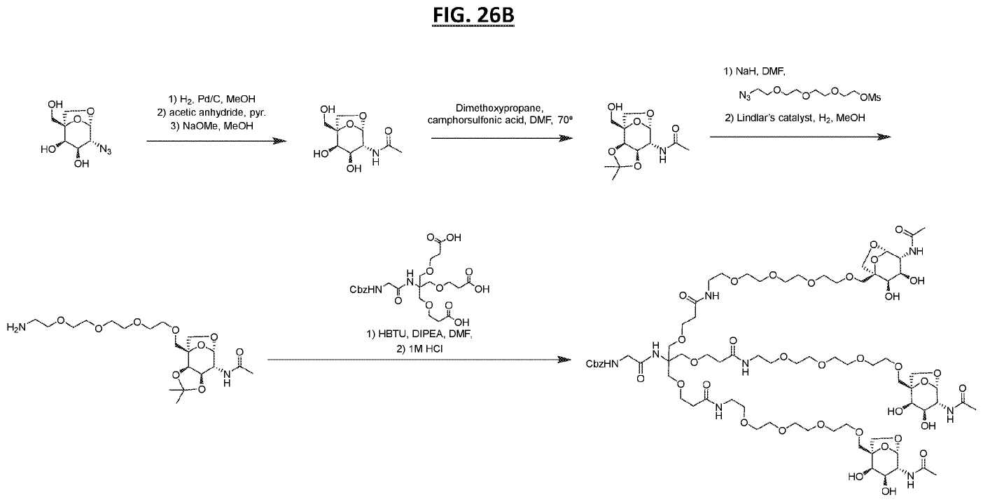

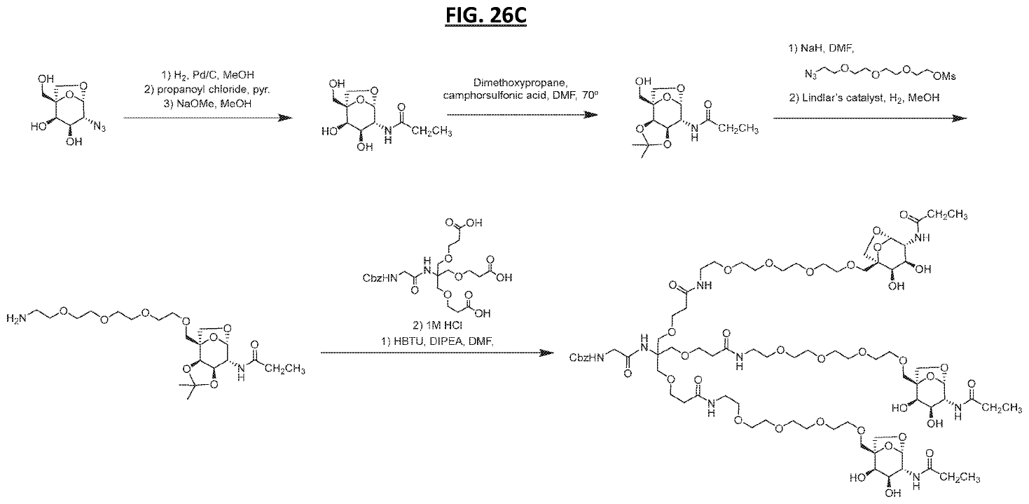

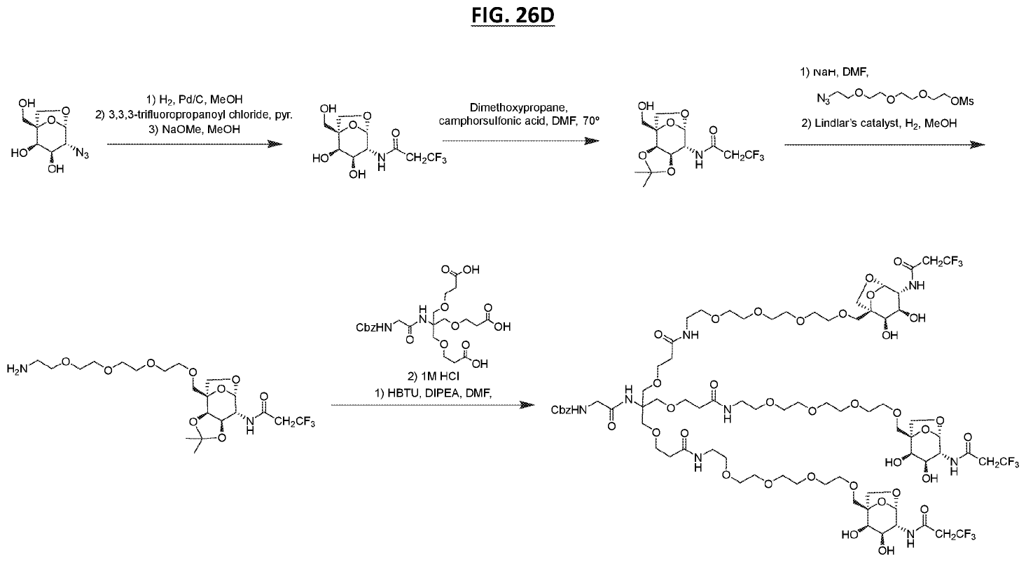

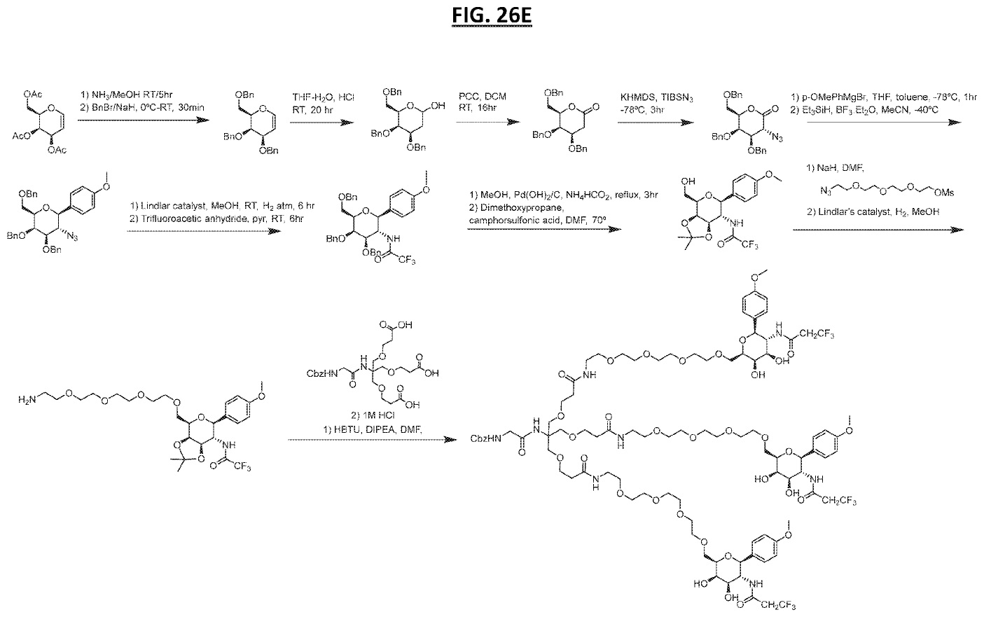

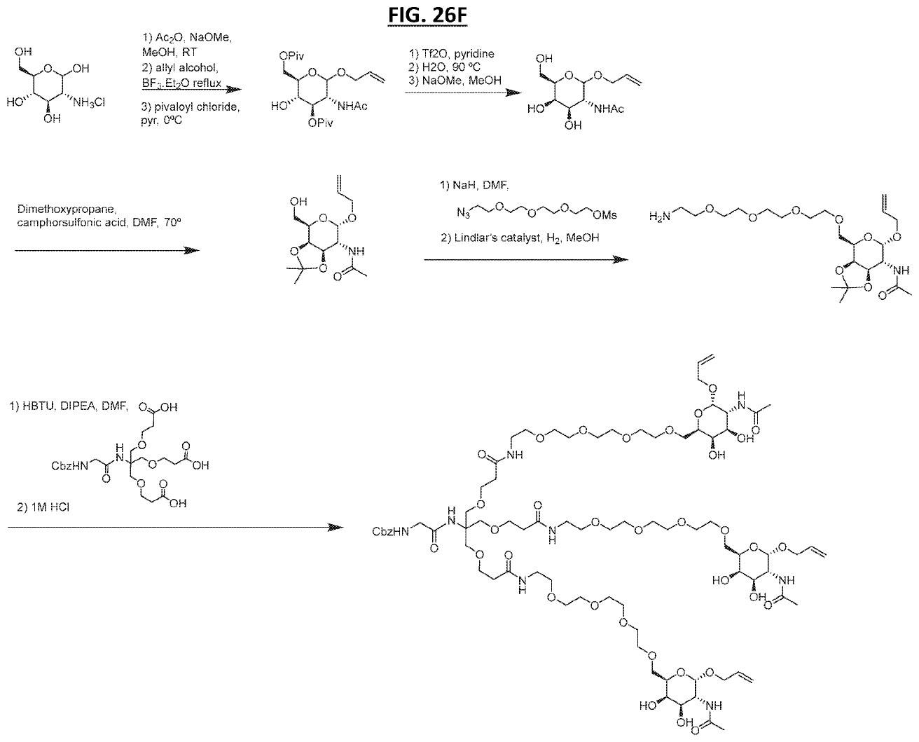

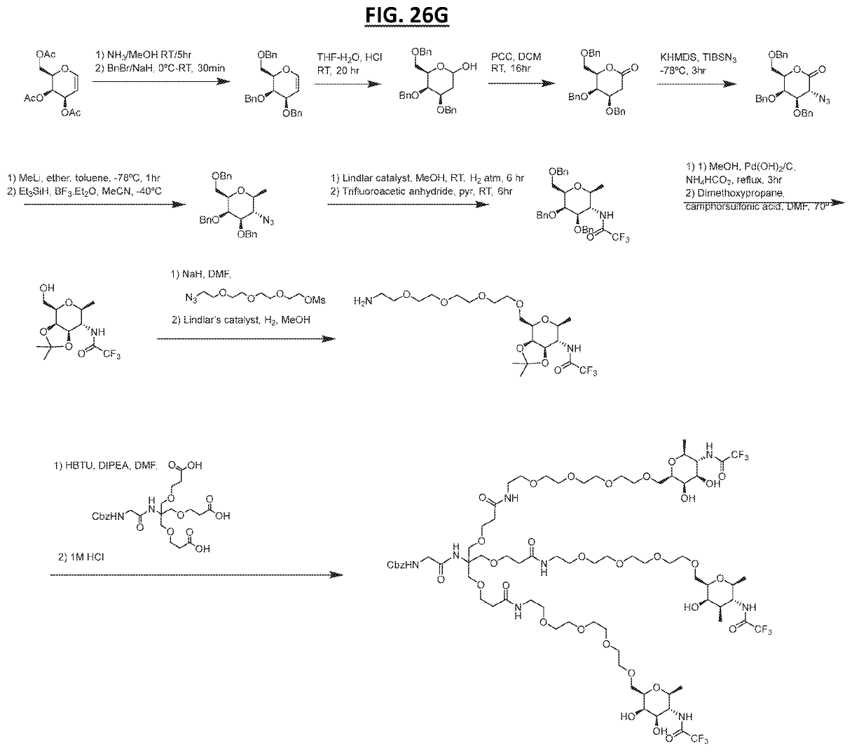

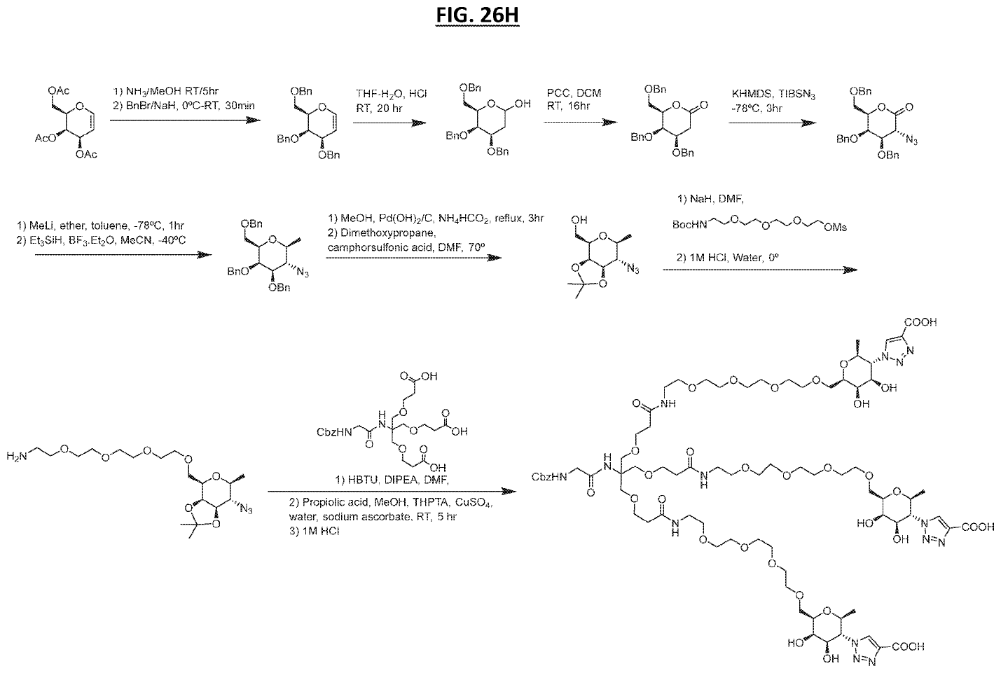

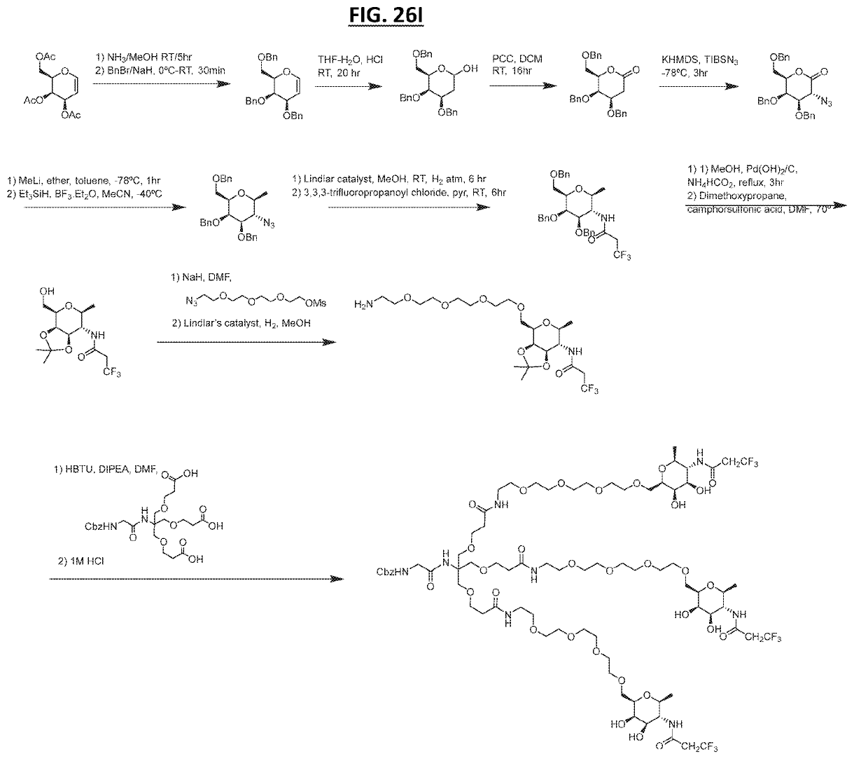

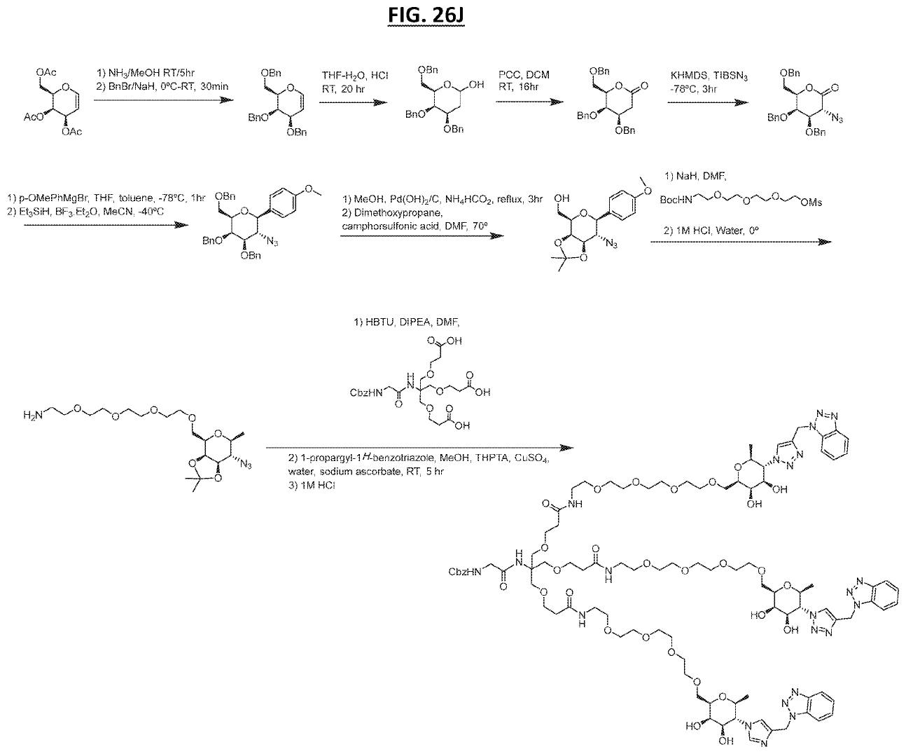

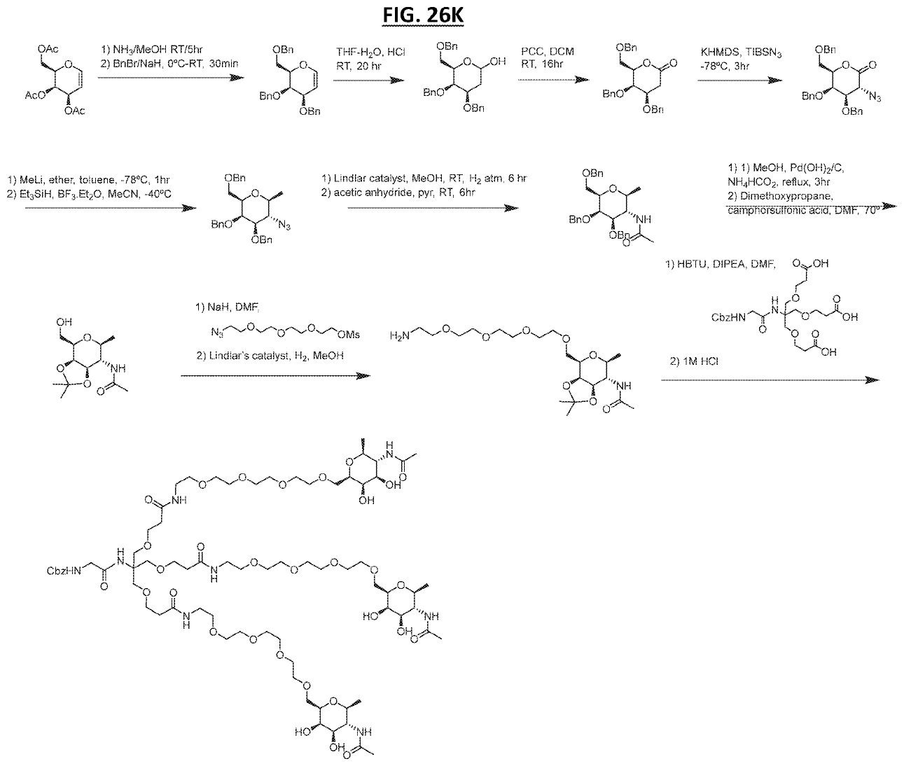

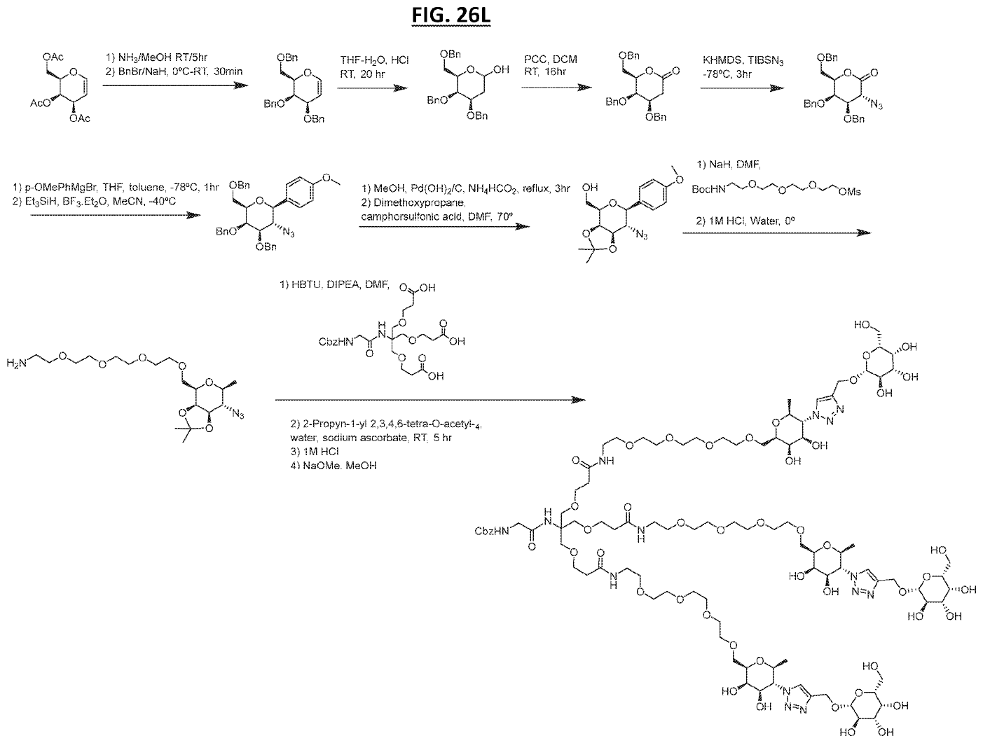

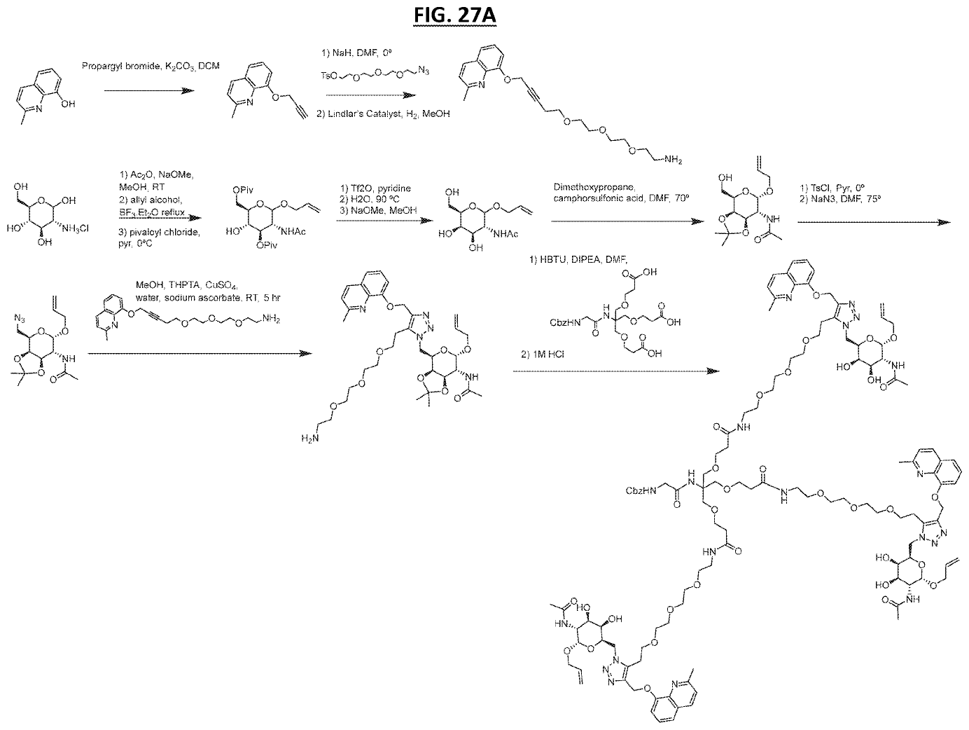

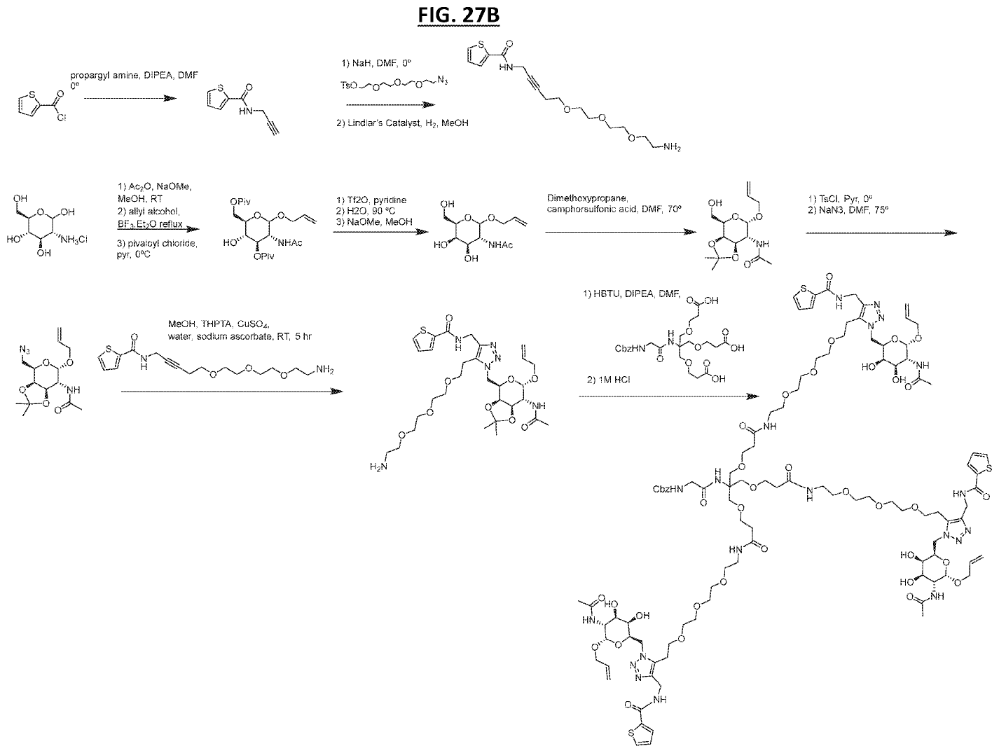

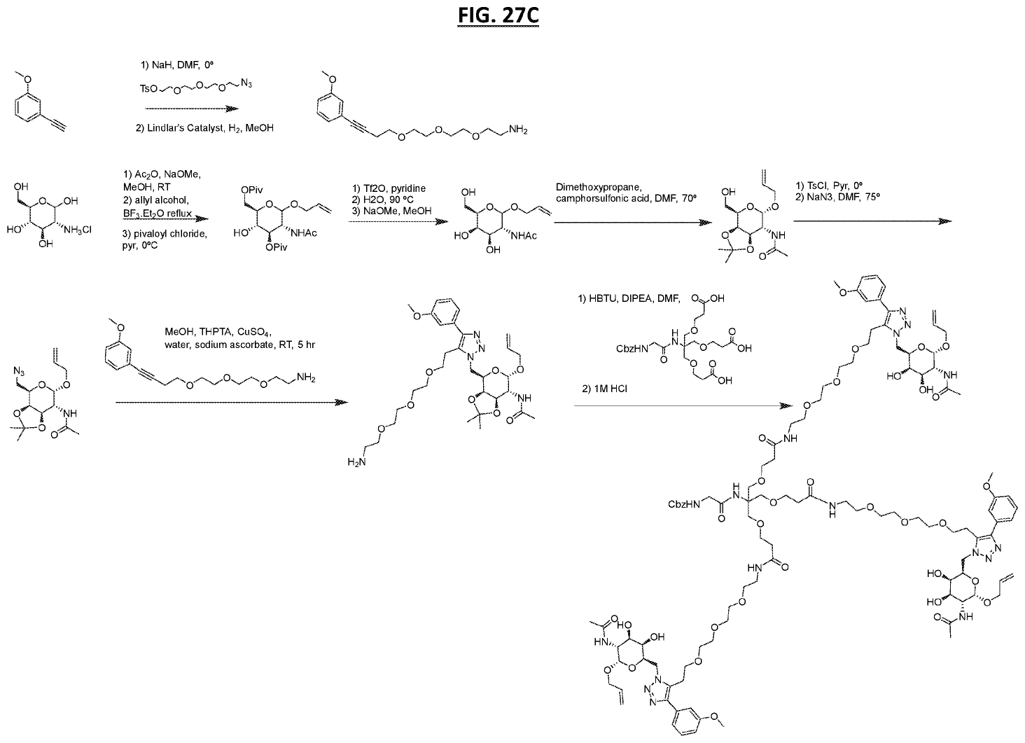

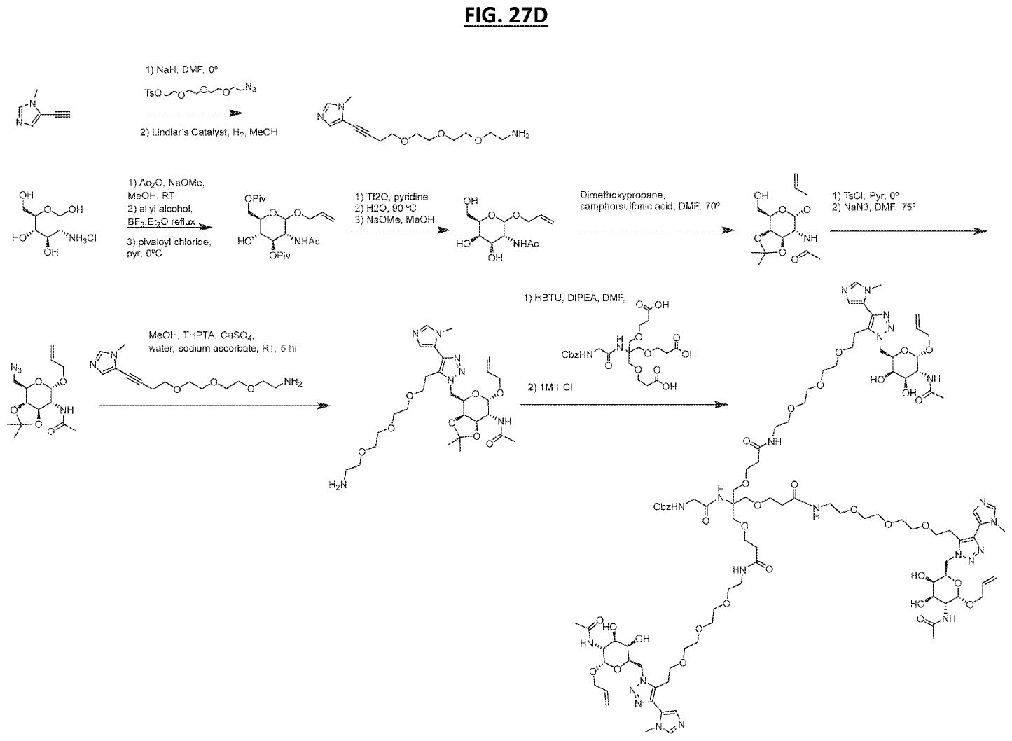

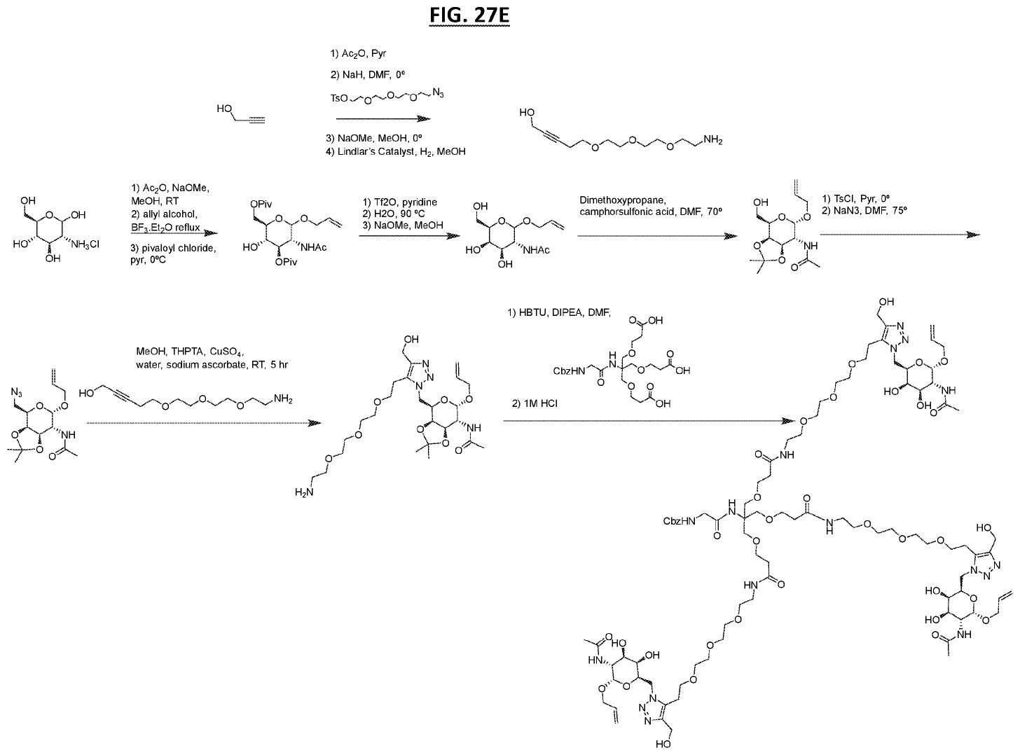

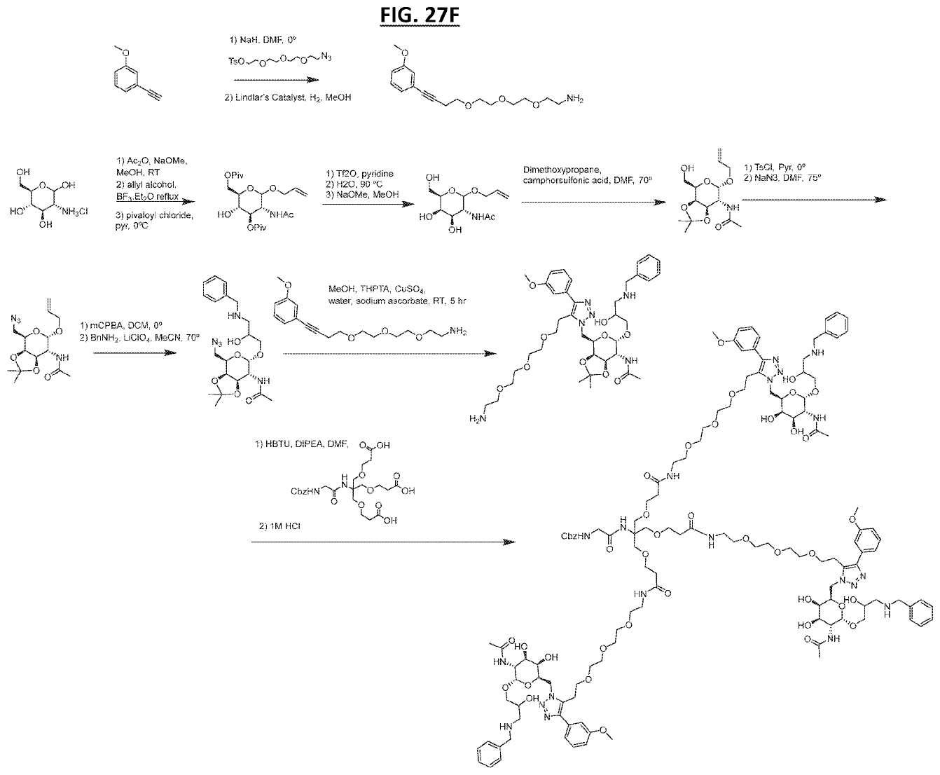

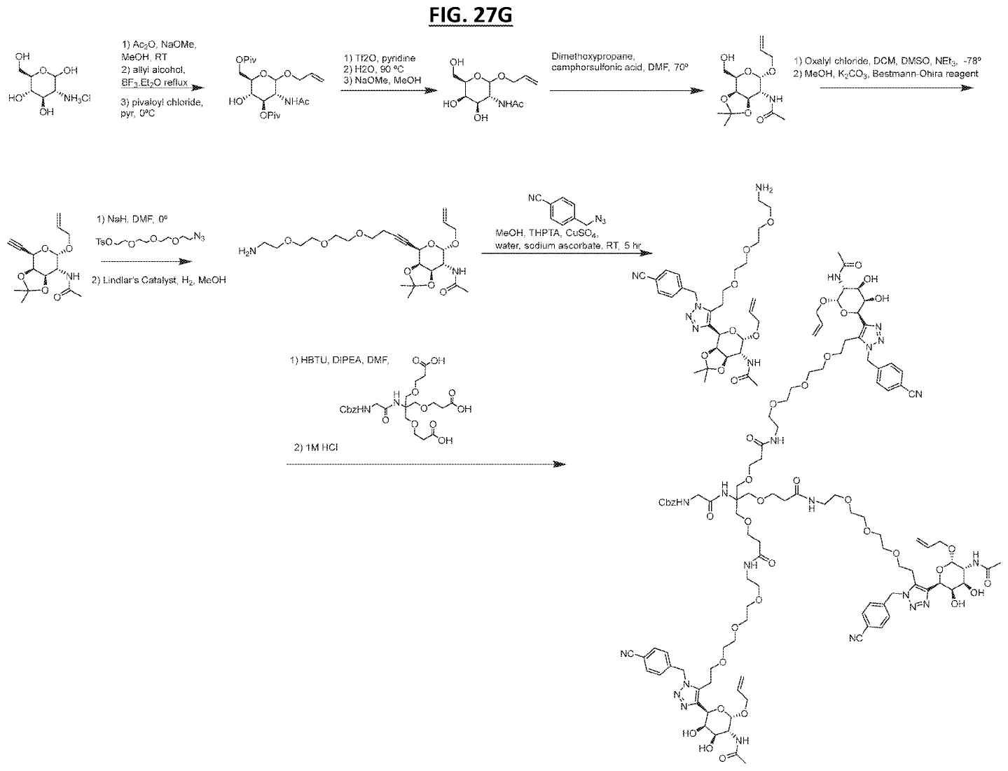

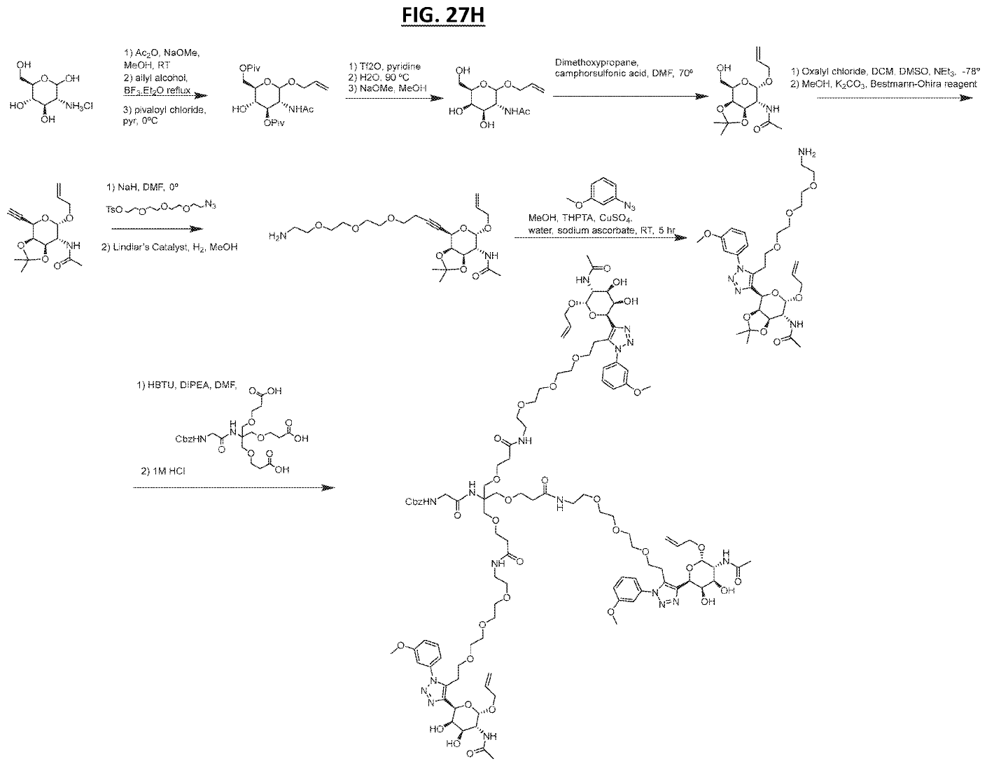

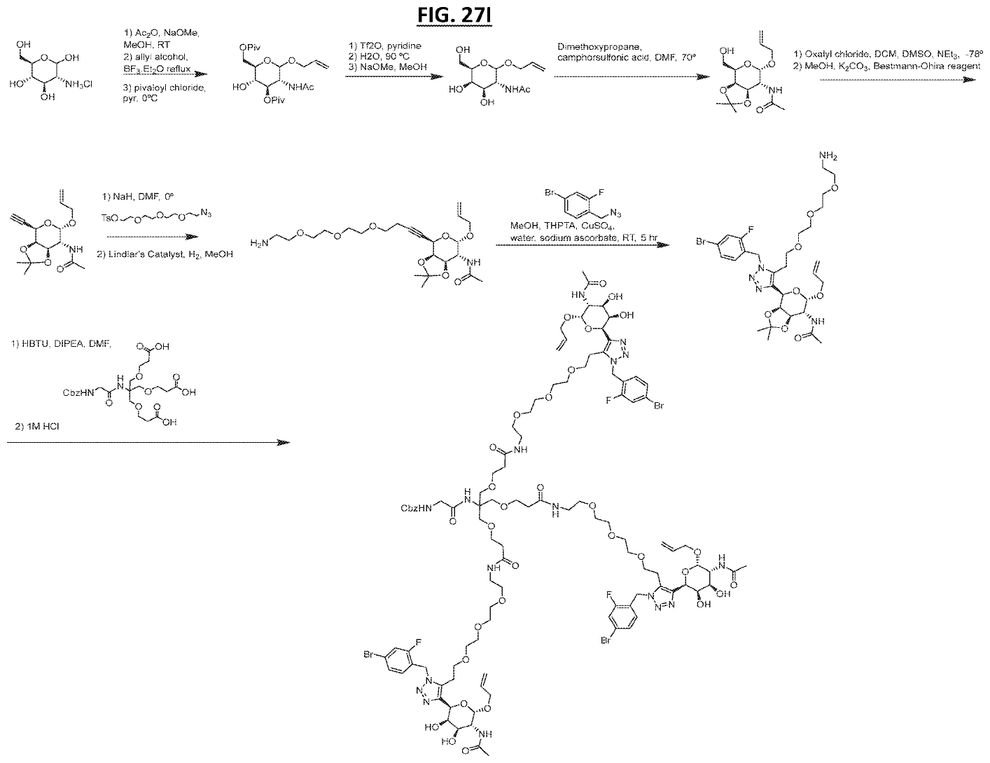

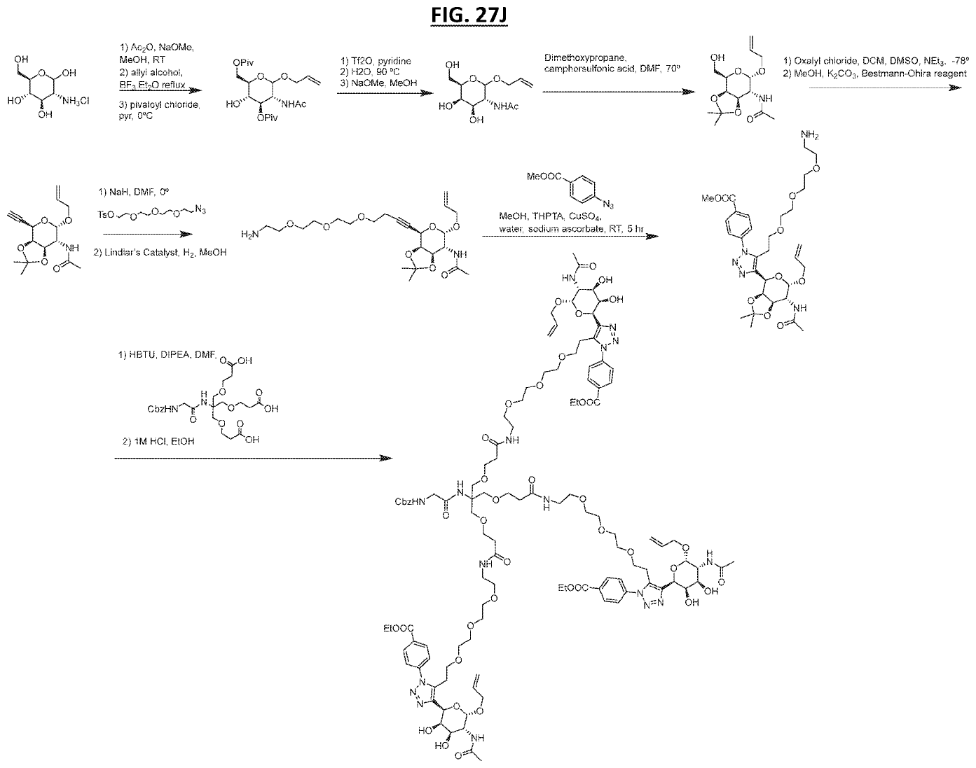

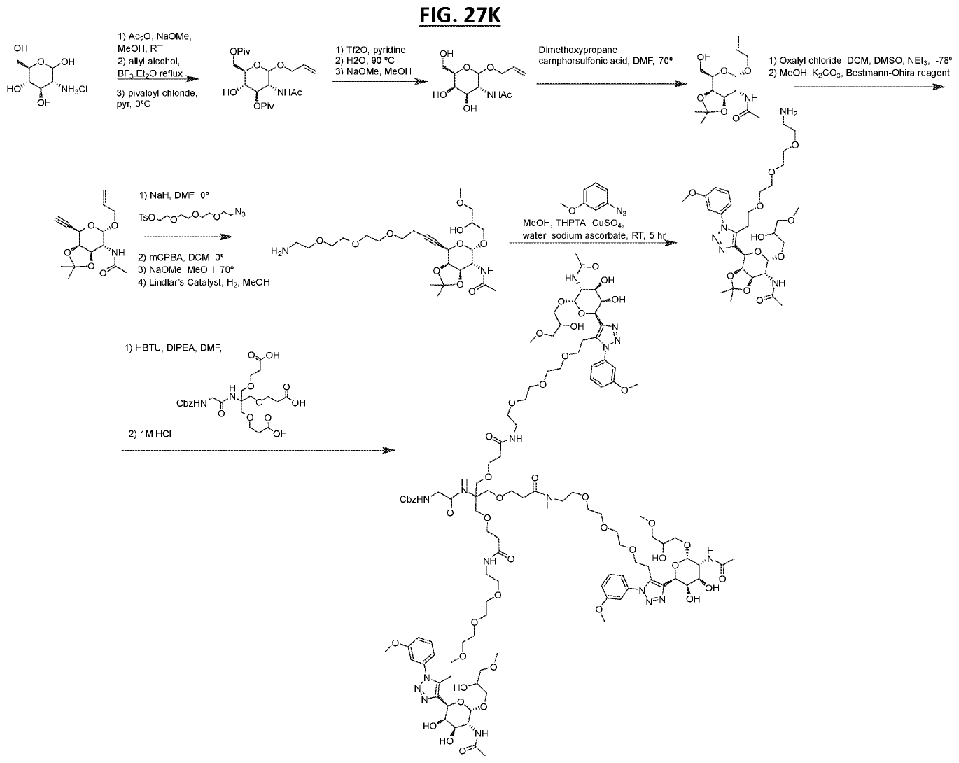

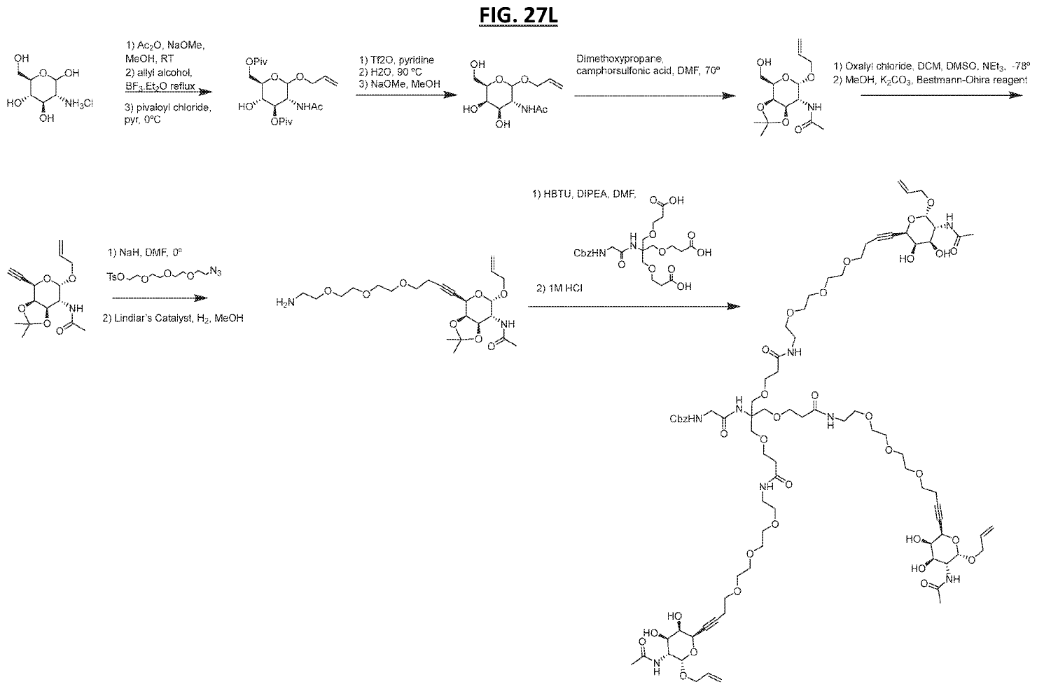

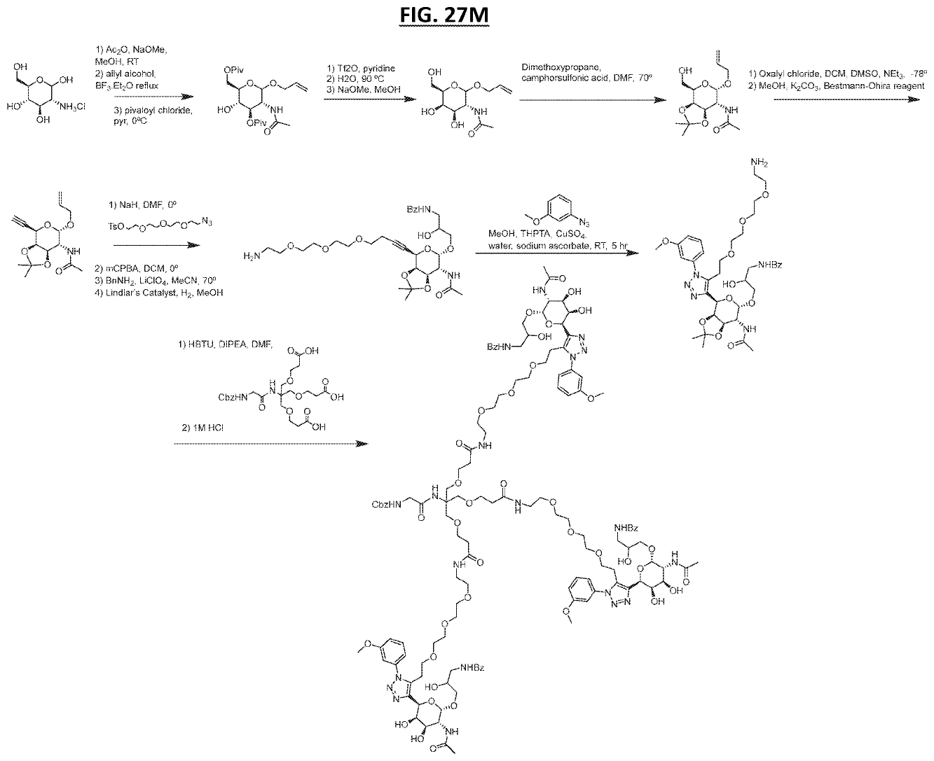

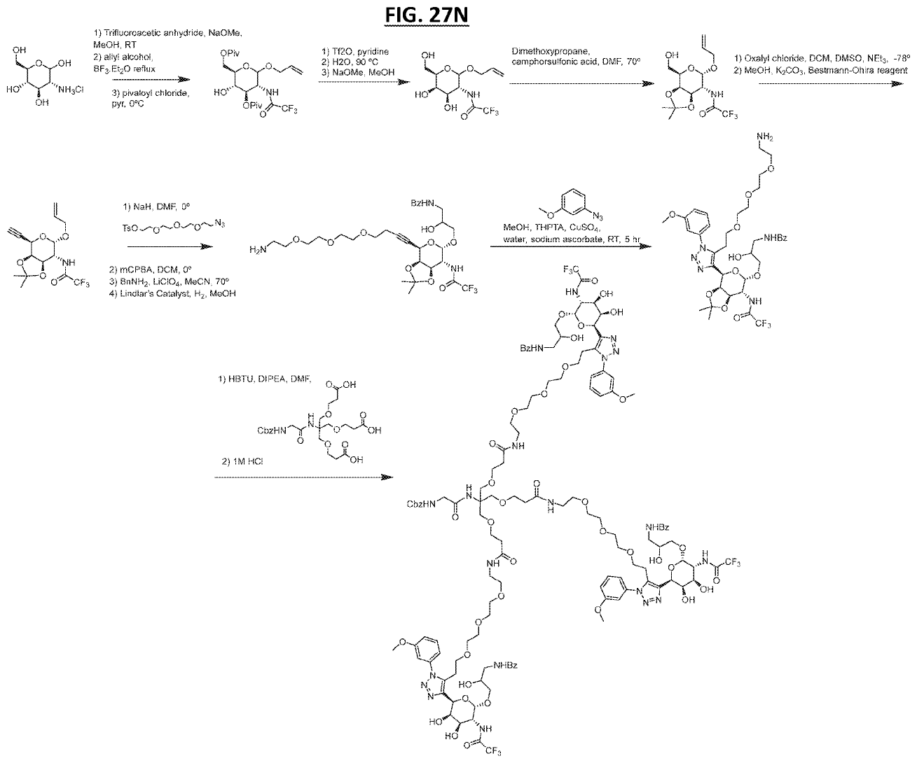

FIGS. 24A-24B, FIGS. 25A-25C, FIGS. 26A-26L, and FIGS. 27A-270 illustrate the non-limiting synthesis of certain ASGPRBM groups which can be used in compounds of formula (I), formula (Ia), formula (II), formula (Ia), formula (III), or formula (IIIa).

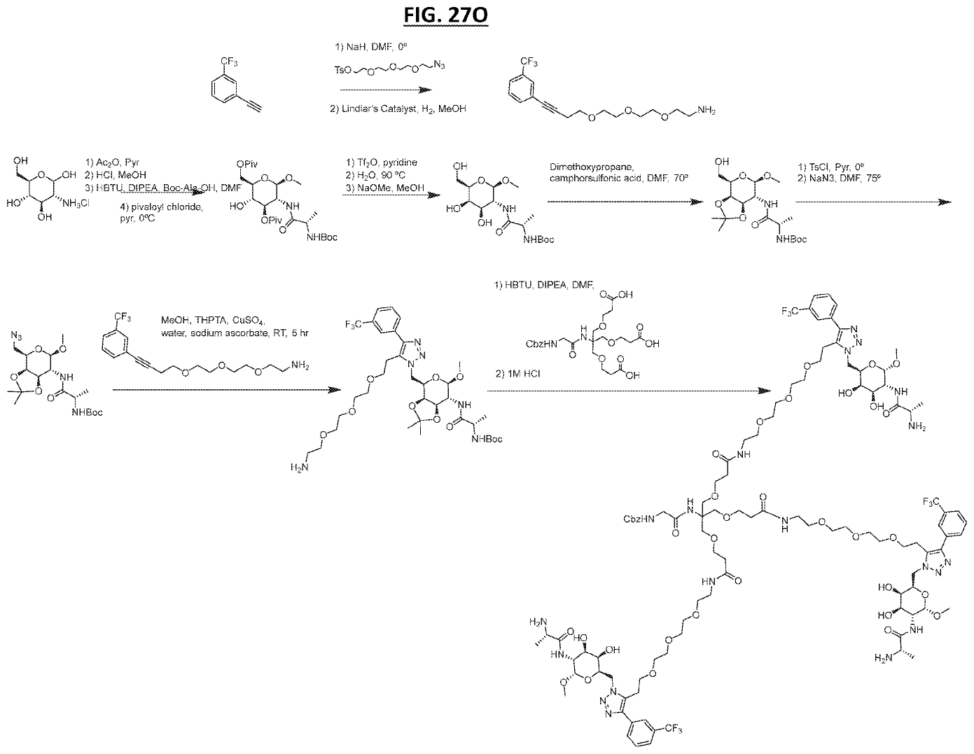

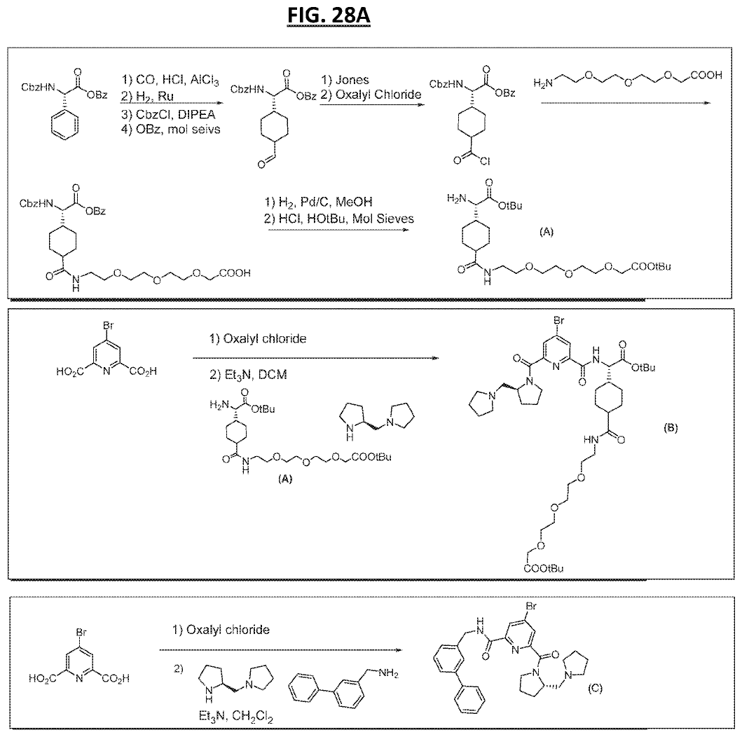

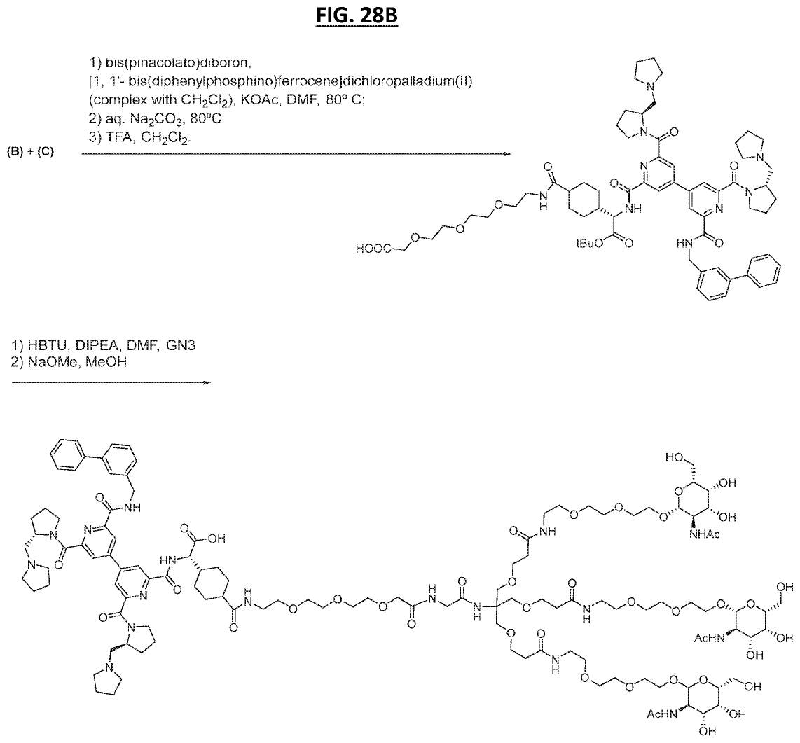

### Example 2: (2S)-2-(2′-(([1,1′-biphenyl]-3-ylmethyl)carbamoyl)-6,6′-bis((S)-2-(pyrrolidin-1-ylmethyl)pyrrolidine-1-carbonyl)-[4,4′-bipyridine]-2-carboxamido)-2-(4-((30-(((2R,3R,4R,5R,6R)-3-acetamido-4,5-dihydroxy-6-(hydroxymethyl)tetrahydro-2H-pyran-2-yl)oxy)-16-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-16-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-11,14,21-trioxo-3,6,9,18,25,28-hexaoxa-12,15,22-triazatriacontyl)carbamoyl)cyclohexyl)acetic acid (FIGS. 28A-28B)

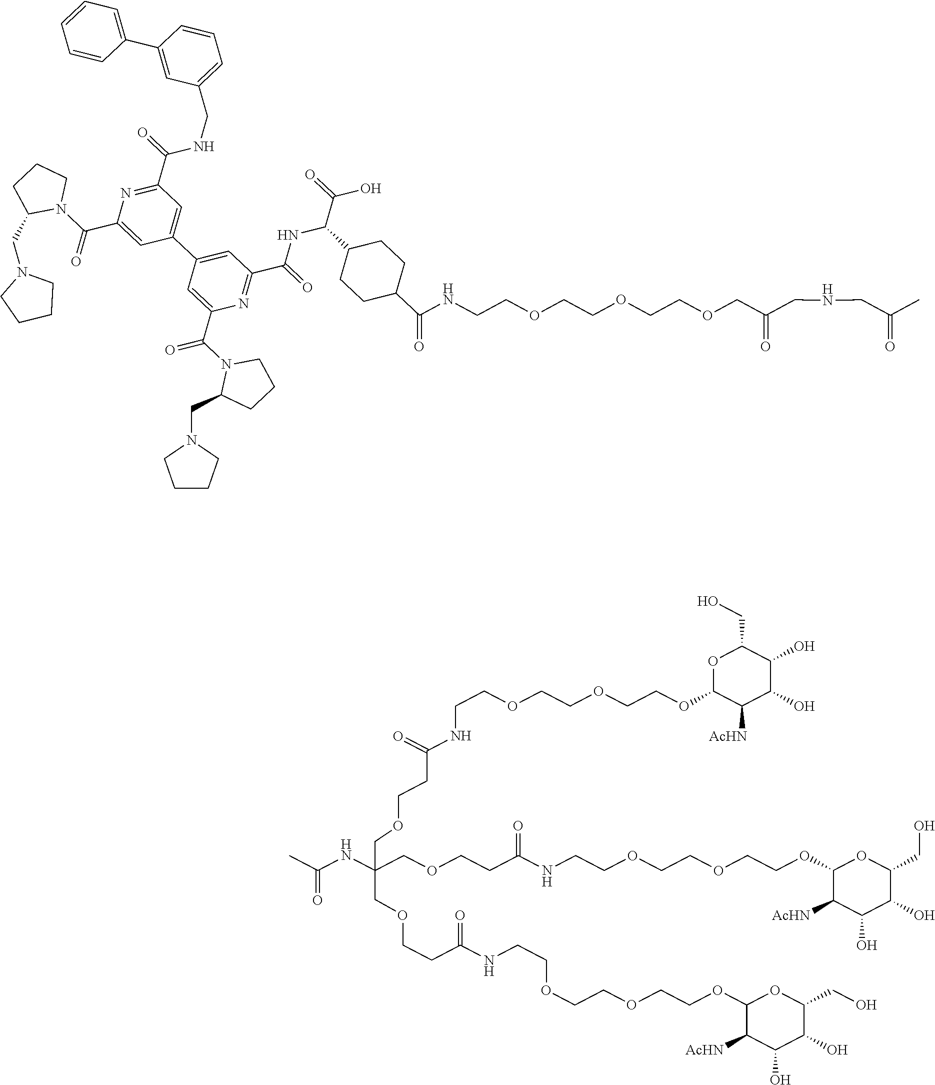

N-Cbz and O-Bz protected phenylglycine was treated with carbon monoxide in the presence of hydrochloric acid and aluminum trichloride to give an intermediate aryl aldehyde, which was then reduced with hydrogen gas over ruthenium to give the cyclohexyl derivative. Any reduced aldehyde was reoxidized using Dess-Martin periodinane. The amine and carboxylic acid were reprotected using CbzCl and Obz respectively. The aldehyde was then oxidized with Jones reagent and reacted with oxaylyl chloride give an acyl chloride, which was reacted with an amine carboxylic acid terminating in a carboxylic acid. The amine and carboxylic acid protecting groups were removed with Pd/C under a hydrogen atmosphere and the carboxylic acids protected with OtBu groups using acid in t-butanol to give intermediate A.

Separately, 4-bromopyridine-2,6-dicarboxylic acid was reacted with oxalyl chloride to give the di-acyl chloride, which was then reacted with intermediate A and (S)-1-(pyrrolidin-2-ylmethyl)pyrrolidine in the presence of triethylamine to give compound B.

Separately, 4-bromopyridine-2,6-dicarboxylic acid was treated with oxalyl chloride and reacted with [1,1′-biphenyl]-3-ylmethanamine and (S)-1-(pyrrolidin-2-ylmethyl)pyrrolidine in the presence of triethylamine to give compound C.

B and C were cross-coupled using a palladium catalyst in DMF at elevated temperature, followed by treatment with aqueous sodium bicarbonate and treatment with TFA in DCM to give a carboxylic acid intermediate. This was then treated with HBTU, DLPEA, and H₂N-GN3 in the presence of DMF. Deprotection with sodium methoxide afforded the final compound.

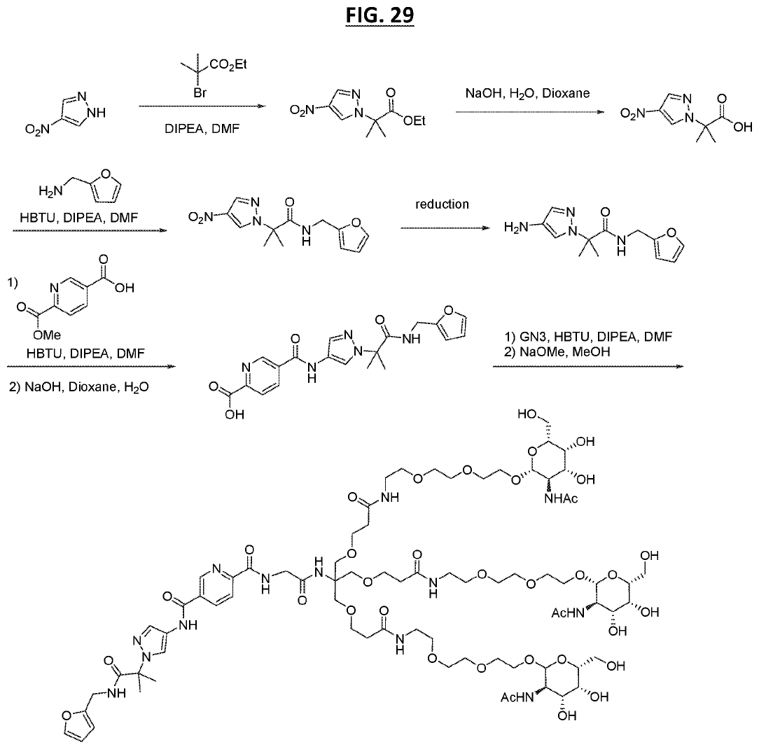

### Example 3: N2-(1-(((2R,3R,4R,5R,6R)-3-acetamido-4,5-dihydroxy-6-(hydroxymethyl)tetrahydro-2H-pyran-2-yl)oxy)-15-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-15-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-10,17-dioxo-3,6,13-trioxa-9,16-diazaoctadecan-18-yl)-N5-(1-(1-((furan-2-ylmethyl)amino)-2-methyl-1-oxopropan-2-yl)-1H-pyrazol-4-yl)pyridine-2,5-dicarboxamide (FIG. 29)

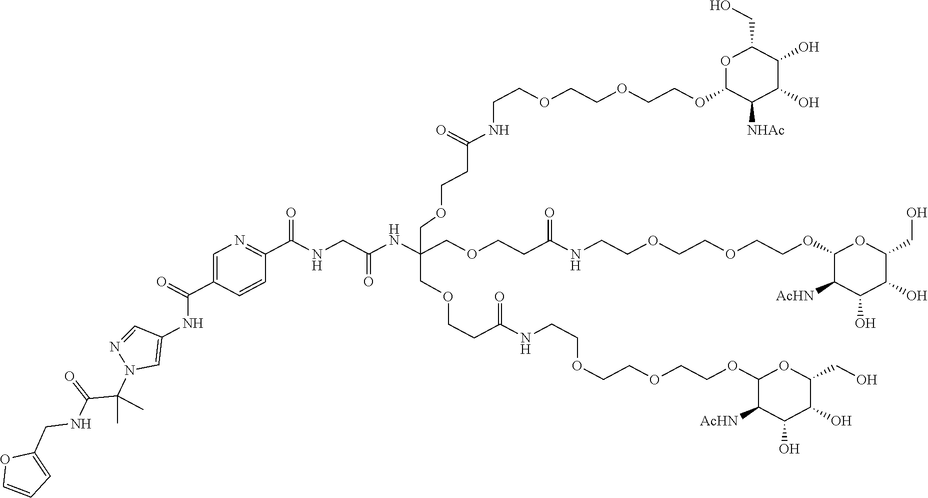 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)c2ccc(C(=O)Nc3cnn(C(C)(C)C(=O)NCc4ccco4)c3)cn2)O[C@H](CO)[C@H](O)[C@@H]1O]

4-nitro-1H-pyrazole was treated with ethyl 2-bromo-2-methylpropanoate in the presence of organic base in DMF to give the alkylated product. The methyl ester was then deprotected with sodium hydroxide, and reacted with furan-2-ylmethanamine under standard amide coupling conditions to give the amide nitro product which was then reduced to afford an amine. This was then reacted with 6-(methoxycarbonyl)nicotinic acid under standard amide coupling conditions to afford the diamide methyl ester, which was deprotected to give the carboxylic acid. This was activated using HBTU and DIPEA in DMF and reacted with H₂N-GN3. Deprotection of the GN3 acyl groups afforded the final compound.

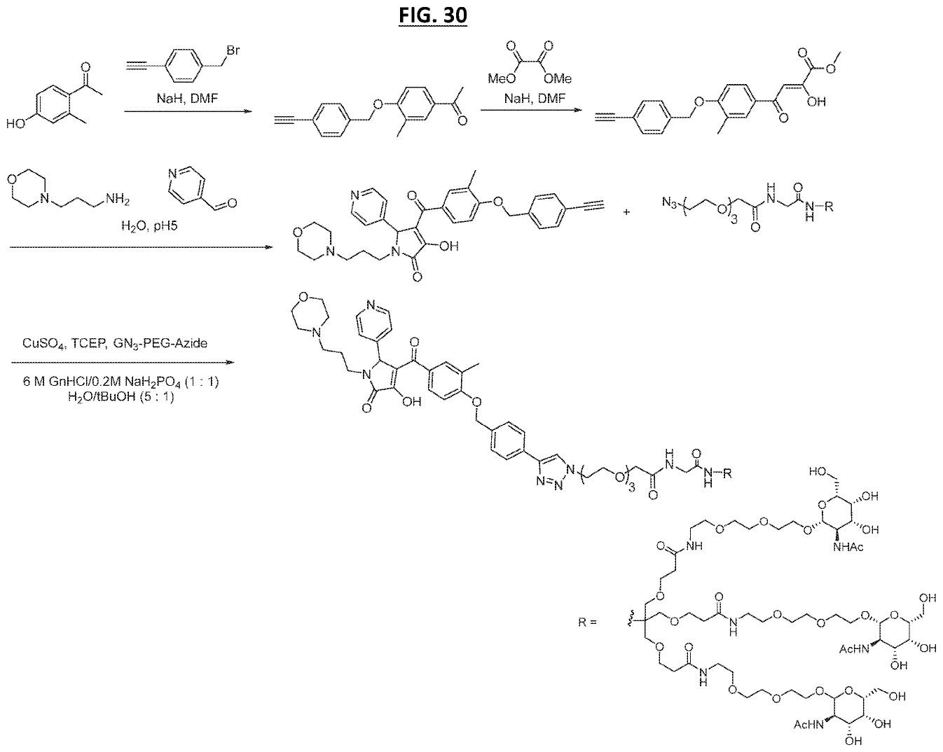

### Example 4: 3,3′-((2-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-2-(14-(4-(4-((4-(4-hydroxy-1-(3-morpholinopropyl)-5-oxo-2-(pyridin-4-yl)-2,5-dihydro-1H-pyrrole-3-carbonyl)-2-methylphenoxy)methyl)phenyl)-1H-1,2,3-triazol-1-yl)-4-oxo-6,9,12-trioxa-3-azatetradecanamido)propane-1,3-diyl)bis(oxy))bis(N-(2-(2-(2-(((2R,3R,4R,5R,6R)-3-acetamido-4,5-dihydroxy-6-(hydroxymethyl)tetrahydro-2H-pyran-2-yl)oxy)ethoxy)ethoxy)ethyl)propanamide) (FIG. 30)

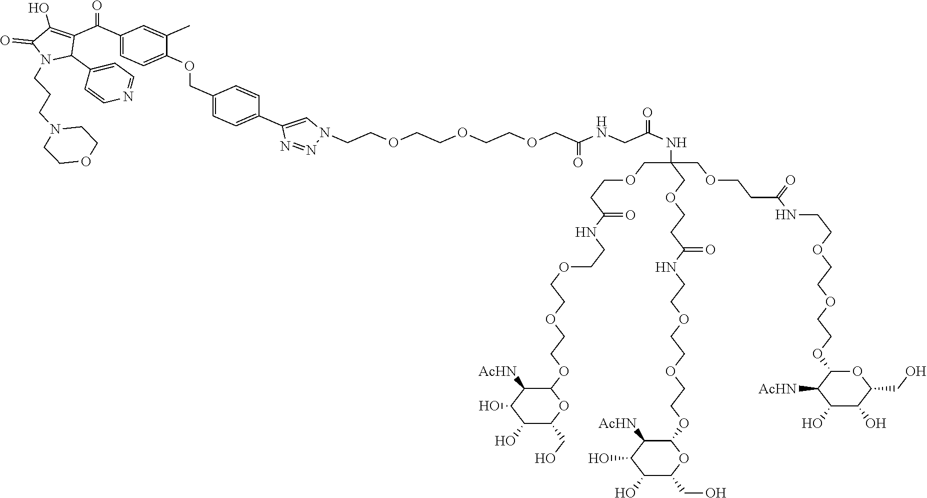 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)COCCOCCOCCn2cc(-c3ccc(COc4ccc(C(=O)C5=C(O)C(=O)N(CCCN6CCOCC6)C5c5ccncc5)cc4C)cc3)nn2)O[C@H](CO)[C@H](O)[C@@H]1O]

1-(4-hydroxy-2-methylphenyl)ethan-1-one was treated with sodium hydride and reacted with 1-(bromomethyl)-4-ethynylbenzene to give the intermediate ketone, which was reacted with sodium hydride and treated with dimethyl oxalate to give the enol product. This was then condensed with 3-morpholinopropan-1-amine and isonicotinaldehyde to form a 5-membered ring. The resulting alkyne was reacted with N₃—(CH₂CH₂O)₃CH₂C(═O)NH-GN3 (acyl-deprotected) under standard click coupling conditions to afford the product.

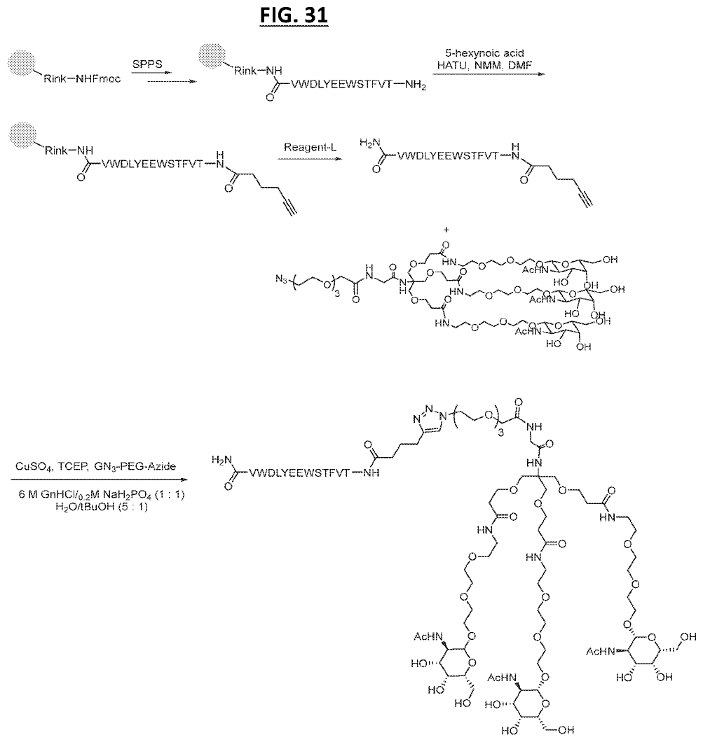

### Example 5 (FIG. 31)

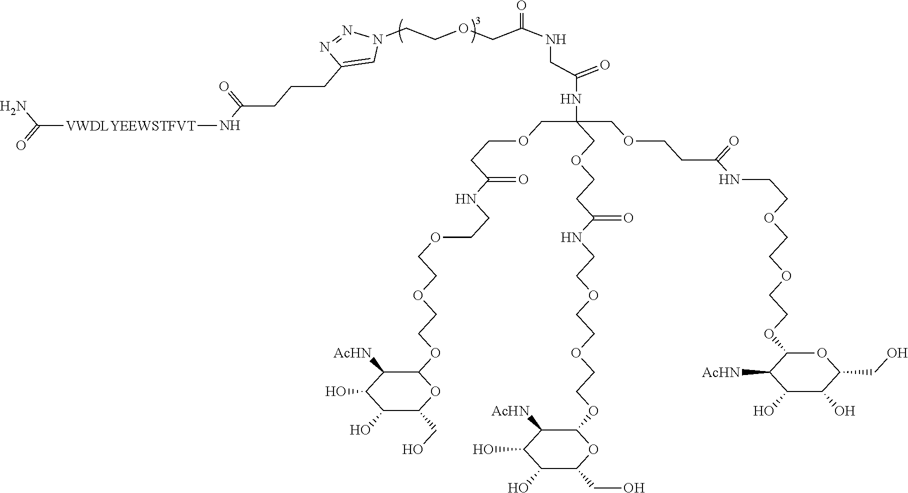

Peptide VWDLYEEWSTFVT (SEQ ID NO: 135) was synthesized according to standard Fmoc protocols. The peptide was treated with 5-hexynoic acid on resin to afford the alkyne, which was cleaved from resin using Reagent L. The alkyne was reacted with N₃—(CH₂CH₂O)₃CH₂C(═O)NH-GN3 (deprotected OAc groups with sodium methoxide) under standard copper-mediated conditions to give the final bifunctional molecule.

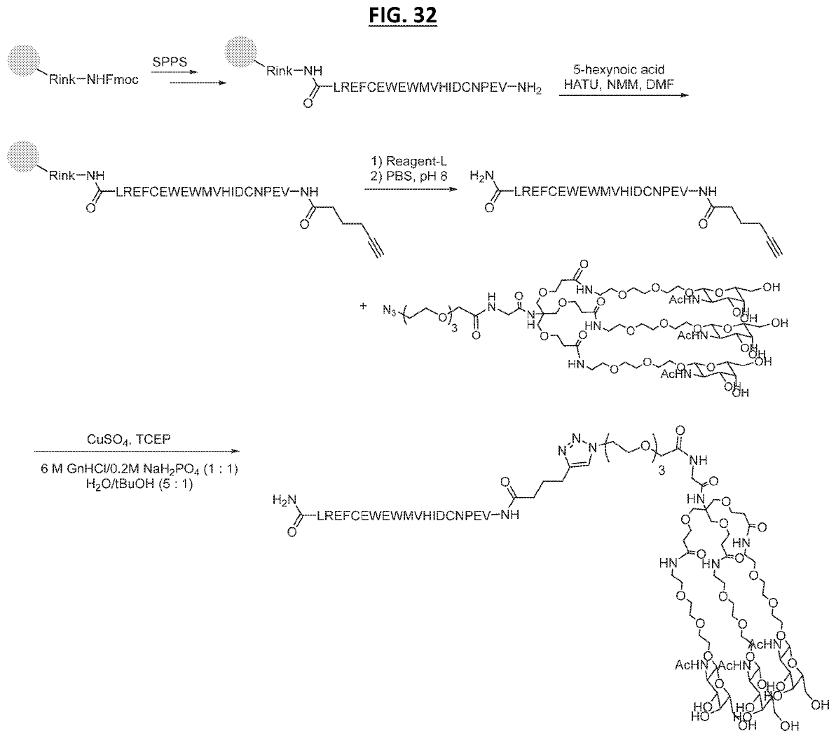

### Example 6 (FIG. 32)

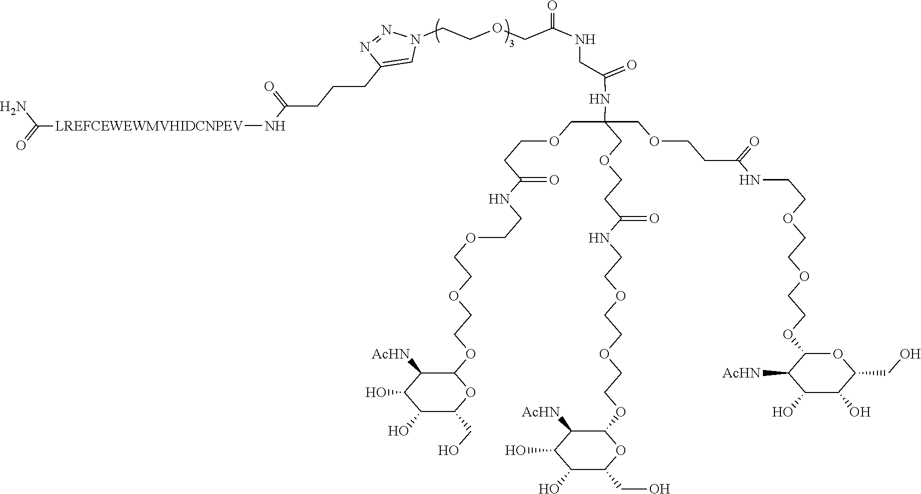

Peptide LREFCEWEWMVHIDCNPEV (SEQ ID NO: 136) was synthesized according to standard Fmoc protocols. The peptide was treated with 5-hexynoic acid on resin to afford the alkyne, which was cleaved from resin using Reagent L, then cyclized overnight in pH 8 buffer. The alkyne was reacted with N₃—(CH₂CH₂O)₃CH₂C(═O)NH-GN3 (deprotected OAc groups with sodium methoxide) under standard copper-mediated conditions to give the final bifunctional molecule.

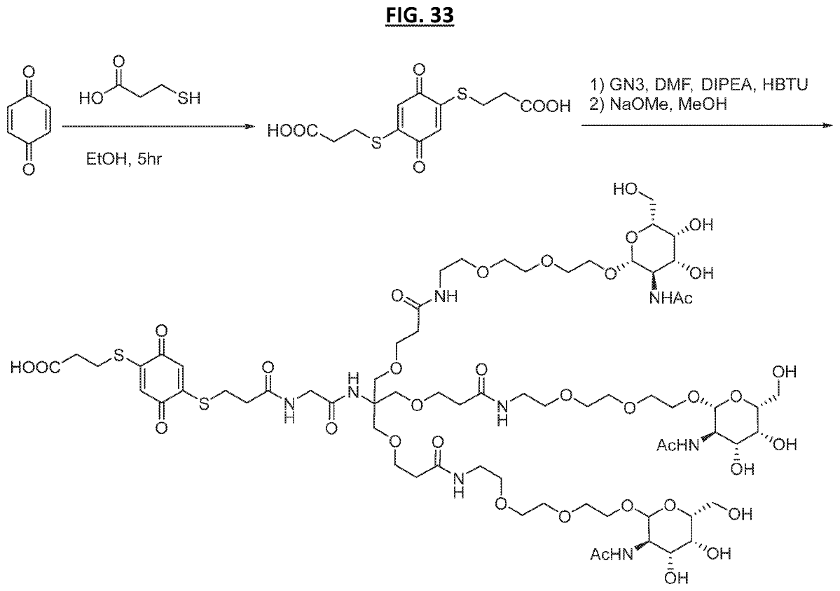

### Example 7: 3-((4-((1-(((2R,3R,4R,5R,6R)-3-acetamido-4,5-dihydroxy-6-(hydroxymethyl)tetrahydro-2H-pyran-2-yl)oxy)-15-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-15-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-10,17,20-trioxo-3,6,13-trioxa-9,16,19-triazadocosan-22-yl)thio)-3,6-dioxocyclohexa-1,4-dien-1-yl)thio)propanoic acid (FIG. 33)

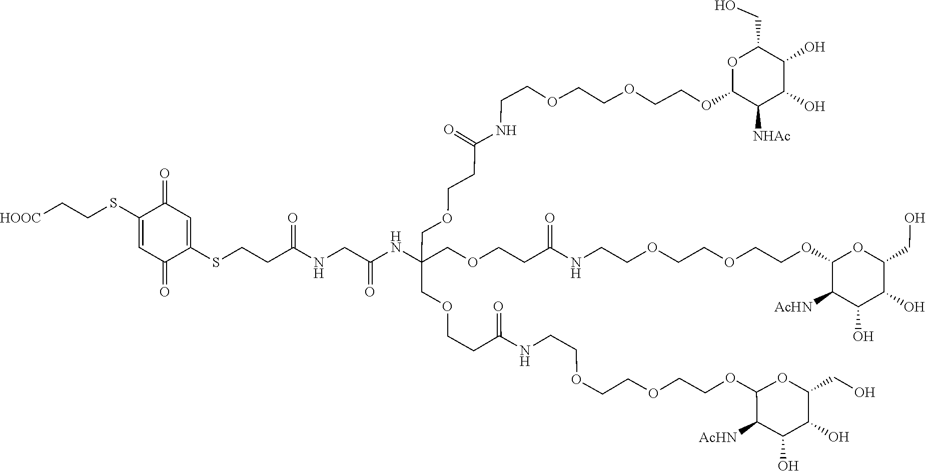 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)CCSC2=CC(=O)C(SCCC(=O)O)=CC2=O)O[C@H](CO)[C@H](O)[C@@H]1O]

Benzoquinone was reacted with two equivalents of 3-mercaptopropanoic acid to give the dicarboxylic acid. This was then reacted with 1 equivalent of HBTU in the presence of organic base in DMF and treated with H₂N-GN3 (acyl deprotected with sodium methoxide) to give the bifunctional molecule.

### Example 8 (FIG. 34)

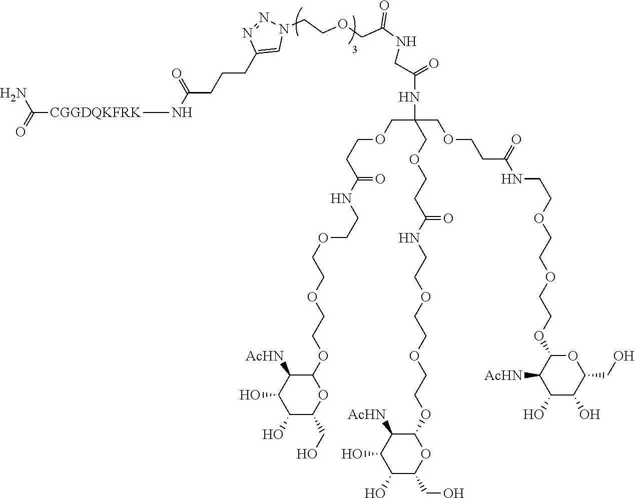

Peptide CGGDQKFRK (SEQ ID NO: 137) was synthesized on resin following standard solid phase protocols and treated with 5-hexynoic acid in the presence of HATU, NMM, and DMF. The alkynyl peptide was cleaved from resin using Reagent L and reacted with N₃—(CH₂CH₂O)₃CH₂C(═O)NH-GN3 (acyl deprotected with sodium methoxide) under standard copper-mediated conditions to give the bifunctional molecule.

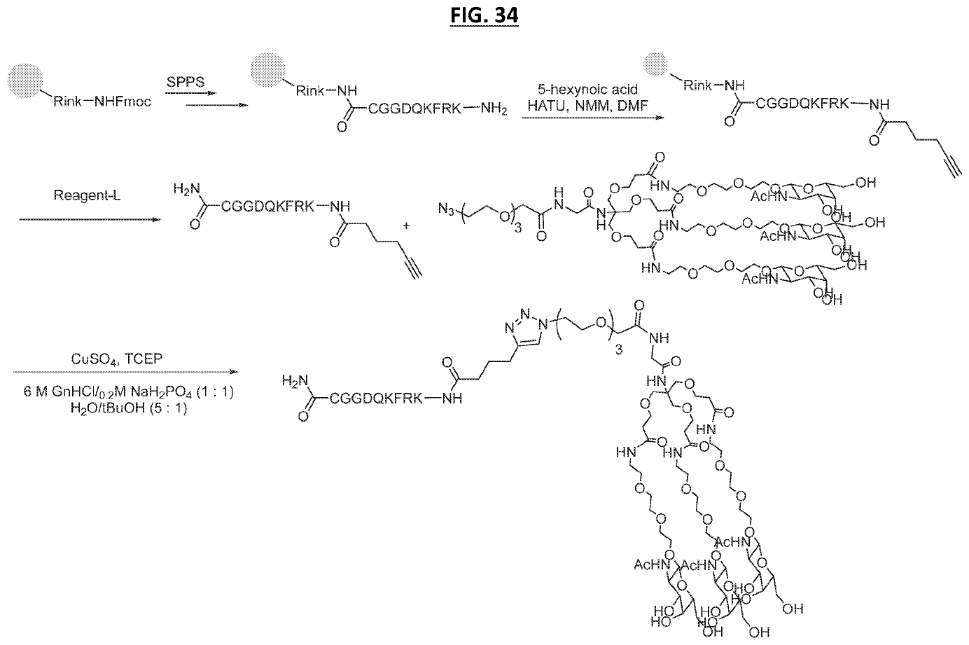

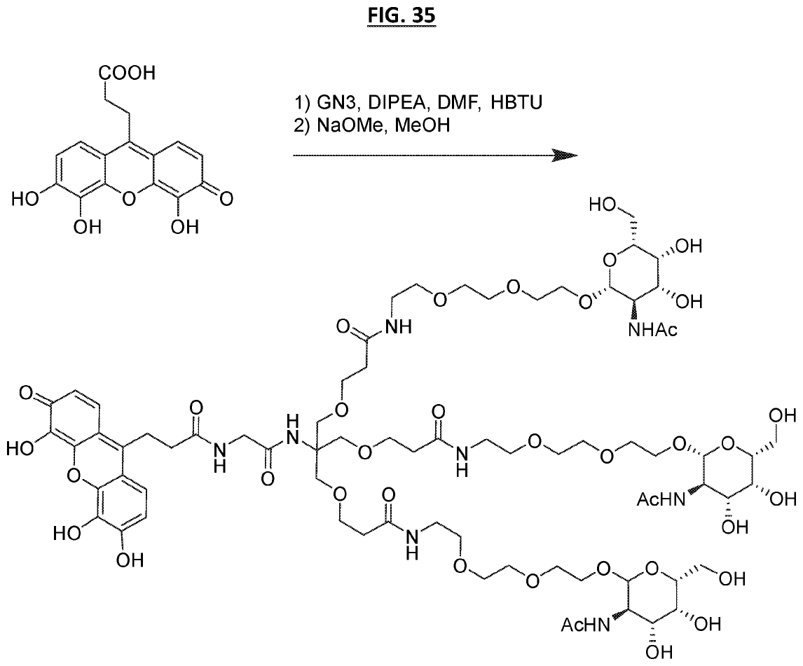

### Example 9: 3,3′-((2-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-2-(2-(3-(4,5,6-trihydroxy-3-oxo-3H-xanthen-9-yl)propanamido)acetamido)propane-1,3-diyl)bis(oxy))bis(N-(2-(2-(2-(((2R,3R,4R,5R,6R)-3-acetamido-4,5-dihydroxy-6-(hydroxymethyl)tetrahydro-2H-pyran-2-yl)oxy)ethoxy)ethoxy)ethyl)propanamide) (FIG. 35)

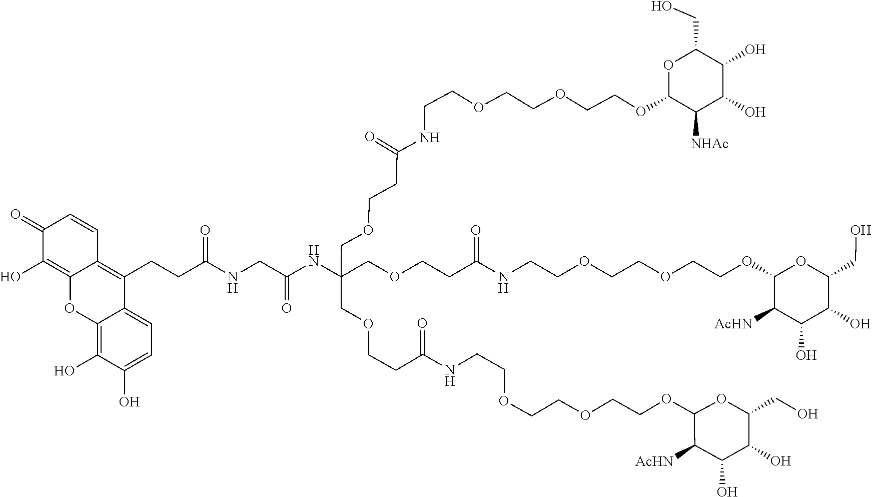 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)CCc2c3ccc(=O)c(O)c-3oc3c(O)c(O)ccc23)O[C@H](CO)[C@H](O)[C@@H]1O]

3-(4,5,6-trihydroxy-3-oxo-3H-xanthen-9-yl)propanoic acid was treated with HBTU in the presence of organic base in DMF and treated with H₂N-GN3 (acetyl deprotected with sodium methoxide) to give the final compound.

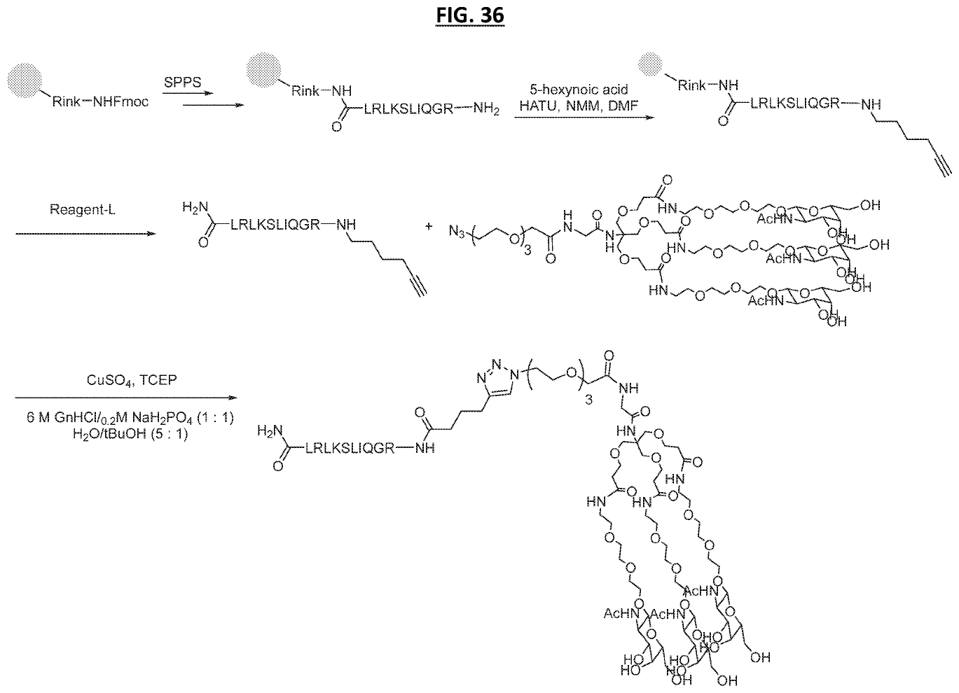

### Example 10 (FIG. 36)

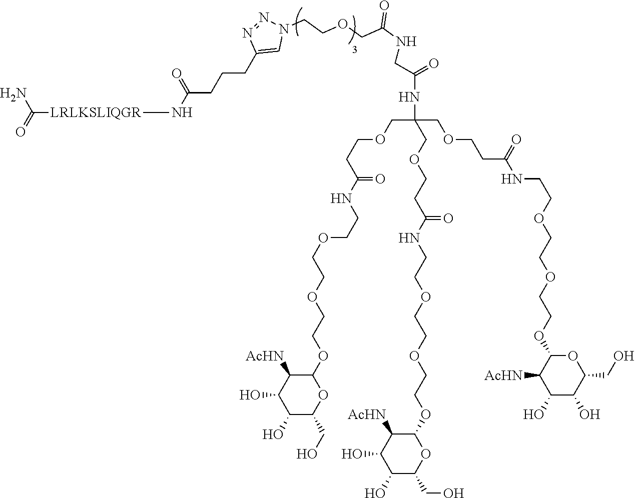

Peptide LRLKSLIQGR (SEQ ID NO:138) was synthesized on resin following standard solid phase protocols and treated with 5-hexynoic acid in the presence of HATU, NMM, and DMF. The alkynyl peptide was cleaved from resin using Reagent L and reacted with N₃—(CH₂CH₂O)₃CH₂C(═O)NH-GN3(acyl deprotected with sodium methoxide) under standard copper-mediated conditions to give the bifunctional molecule.

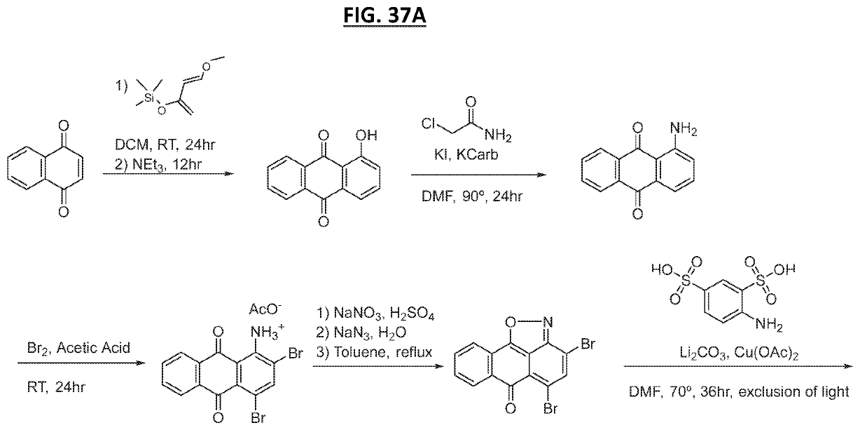

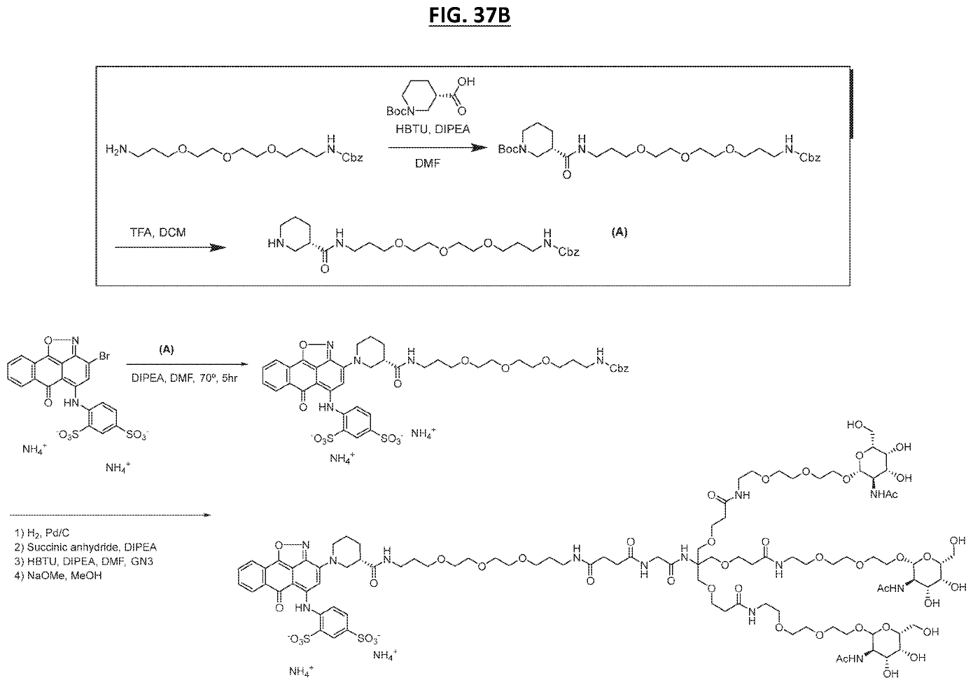

### Example 11: 4-((3-((3S)-3-((1-(((2R,3R,4R,5R,6R)-3-acetamido-4,5-dihydroxy-6-(hydroxymethyl)tetrahydro-2H-pyran-2-yl)oxy)-15-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-15-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-10,17,20,23-tetraoxo-3,6,13,28,31,34-hexaoxa-9,16,19,24-tetraazaheptatriacontan-37-yl)carbamoyl)piperidin-1-yl)-6-oxo-6H-anthra[1,9-cd]isoxazol-5-yl)amino)benzene-1,3-disulfonate (FIG. 37B)

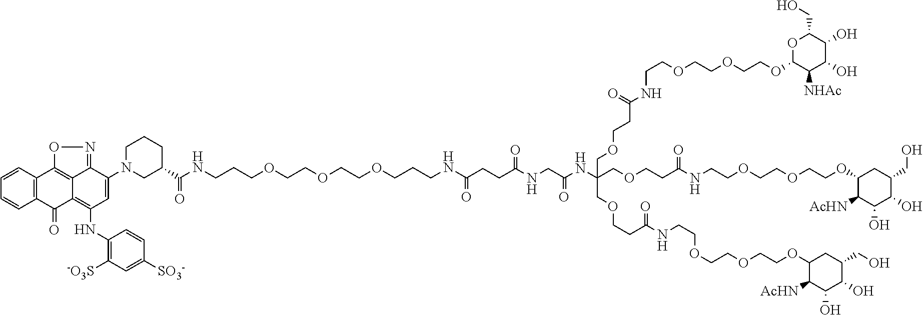

Benzyl (3-(2-(2-(3-aminopropoxy)ethoxy)ethoxy)propyl)carbamate was treated with (S)-1-(tert-butoxycarbonyl)piperidine-3-carboxylic acid under standard amide coupling conditions. This compound was then treated with TFA in DCM to give the monoprotected diamine A.

Separately, naphthalene-1,4-dione was treated with 1-trimethylsiloxy-1,3-butadine in DCM for 24 hours, then treated with triethylamine to afford the aromatic compound (FIG. 37A). This was then treated with chloroacetamide in the presence of potassium iodide and potassium carbonate at elevated temperatures in DMF to afford the amine, which was dibrominated in acetic acid by treatment with elemental bromine. This intermediate was treated with sodium nitrate in strong acid, followed by sodium azide and water and finally refluxed in toluene to afford the cyclized product. This intermediate was then treated with aniline-2,4-disulfonic acid in the presence of lithium carbonate and copper (II) acetate in DMF with the exclusion of light at elevated temperatures.

Treatment with A in the presence of organic base in DMF at elevated temperature gave the Cbz-protected amine, which was deprotected under a hydrogen atmosphere using Pd/C and reacted with succinic anhydride to give a carboxylic acid. This was then treated with HBTU in the presence of organic base in DMF and reacted with H₂N-GN3 (acetyl deprotected with sodium methoxide) to afford the final compound (FIG. 37B).

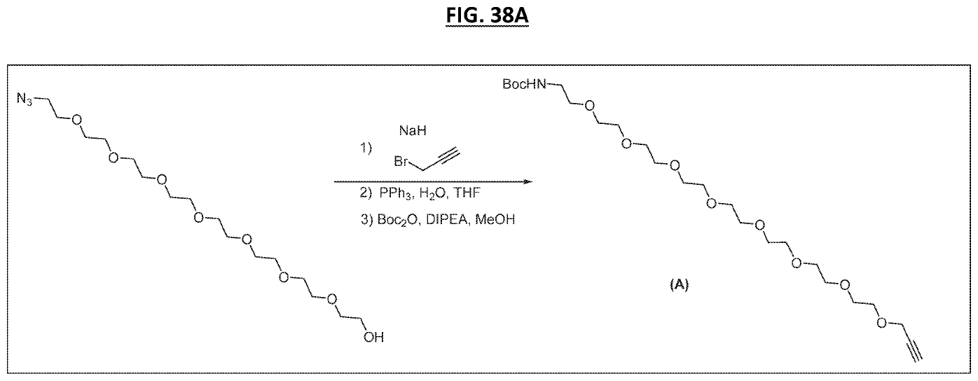

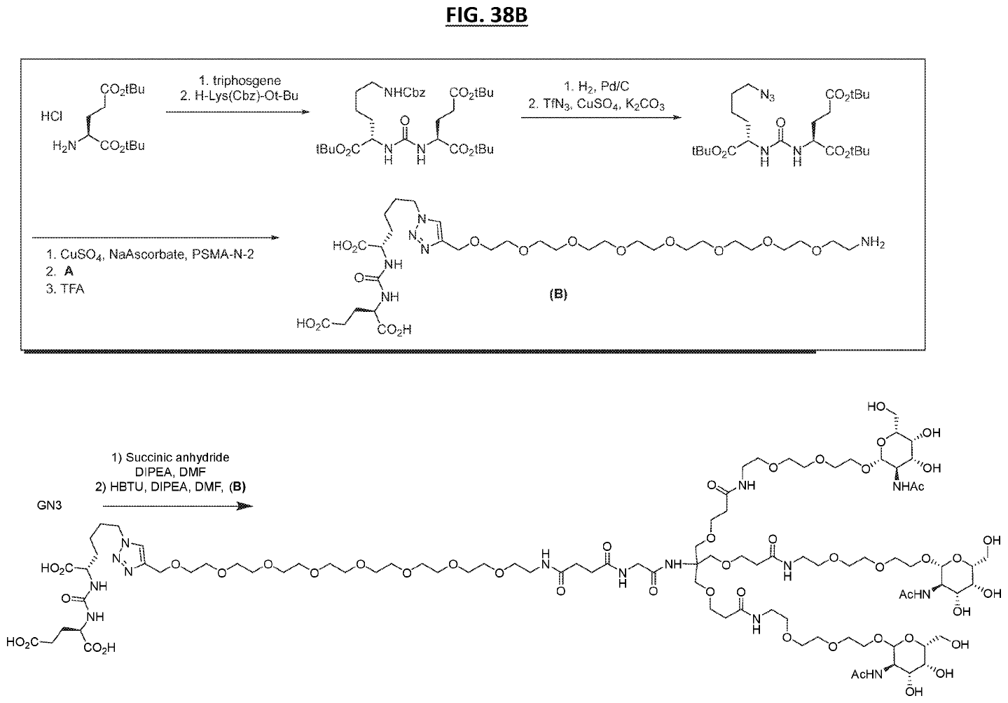

### Example 12: (((1S)-5-(4-(30-((1-(((2R,3R,4R,5R,6R)-3-acetamido-4,5-dihydroxy-6-(hydroxymethyl)tetrahydro-2H-pyran-2-yl)oxy)-15-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-15-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-10,17-dioxo-3,6,13-trioxa-9,16-diazaoctadecan-18-yl)amino)-27,30-dioxo-2,5,8,11,14,17,20,23-octaoxa-26-azatriacontyl)-1H-1,2,3-triazol-1-yl)-1-carboxypentyl)carbamoyl)-D-glutamic acid (FIG. 38B)

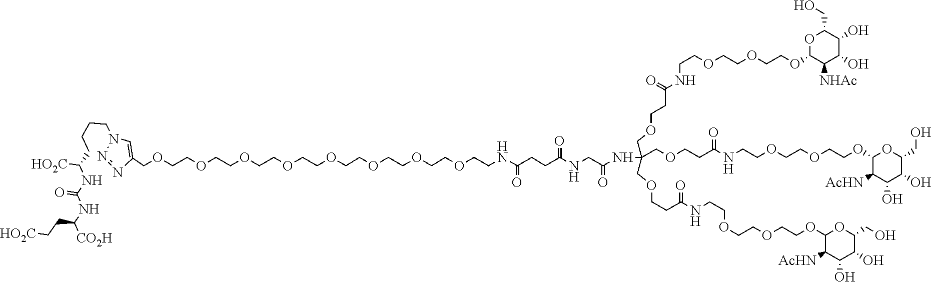 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)CCC(=O)NCCOCCOCCOCCOCCOCCOCCOCCOCc2cn(CCCC[C@H](NC(=O)N[C@H](CCC(=O)O)C(=O)O)C(=O)O)nn2)O[C@H](CO)[C@H](O)[C@@H]1O]

O-(2-Azidoethyl)heptaethylene glycol was treated with sodium hydride and subsequently propargyl bromide to give the intermediate alkyne, which was then reduced using triphenyl phosphine in water/THF, followed by protection of the amine with Boc anhydride in the presence of organic base in methanol, affording A (FIG. 38A).

Separately, di-tert-butyl L-glutamate was treated with triphosgene followed by H-Lys(cbz)-Ot-Bu to give the urea intermediate. This was then reduced with hydrogen gas atmosphere over Pd/C to give the intermediate amine, which was converted to an azide by treatment with triflic azide and copper in the presence of base. This azide was then reacted with intermediate A under standard copper mediated cyclization conditions, then treated with TFA to provide amine B.

Separately, H₂N-GN3 (acetyl deprotected using sodium methoxide in methanol) was treated with succinic anhydride in the presence of organic base in DMF to give a carboxylic acid, which was then activated with HBTU in the presence of DIPEA in DMF to react with amine B to give the final molecule (FIG. 38B).

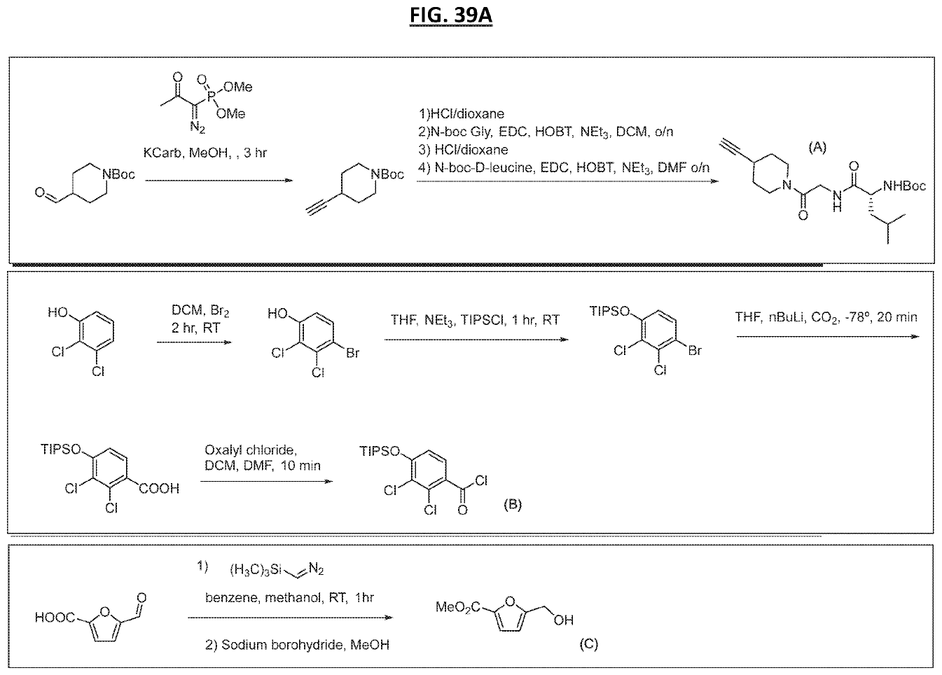

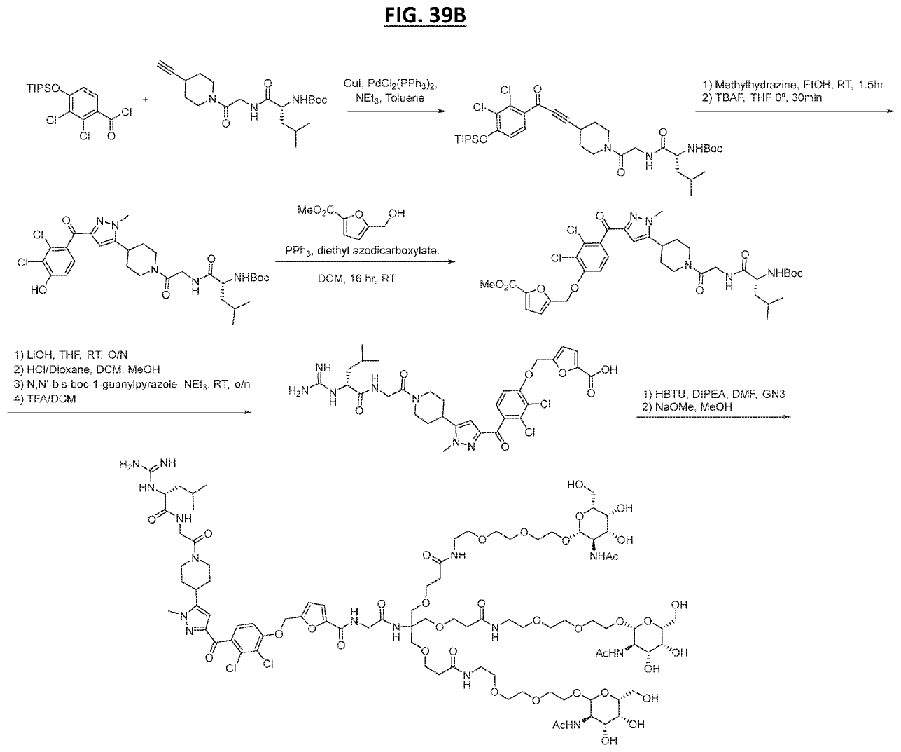

### Example 13: 3,3′-((2-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-2-(2-(5-((2,3-dichloro-4-(5-(1-(2-((R)-2-guanidino-4-methylpentanamido)acetyl)piperidin-4-yl)-1-methyl-1H-pyrazole-3-carbonyl)phenoxy)methyl)furan-2-carboxamido)acetamido)propane-1,3-diyl)bis(oxy))bis(N-(2-(2-(2-(((2R,3R,4R,5R,6R)-3-acetamido-4,5-dihydroxy-6-(hydroxymethyl)tetrahydro-2H-pyran-2-yl)oxy)ethoxy)ethoxy)ethyl)propanamide) (FIG. 39A-39B)

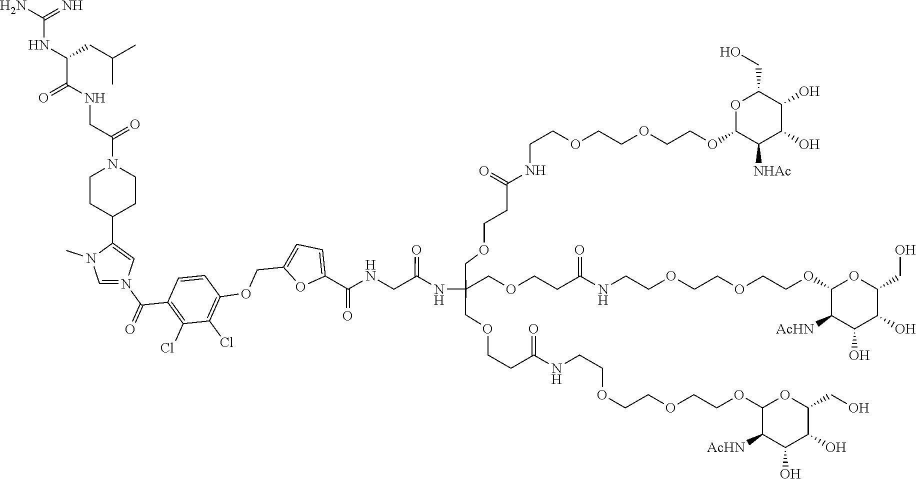

Tert-butyl 4-formylpiperidine-1-carboxylate was treated with dimethyl-1-diazo-2-oxopropylphosphonate in the presence of potassium carbonate in methanol to afford the resulting alkyne amine, which was deprotected using acid in dioxane to afford the resulting amine. This was reacted under standard coupling conditions with N-Boc glycine to afford the Boc-protected amine, which was deprotected as above and coupled to N-Boc-D-leucine using standard coupling conditions. The resulting alkyne is intermediate A.

Separately, 2,3-dichlorophenol was treated with bromine in DCM for 2 hours to afford the mono-brominated product. This was then reacted with TIPS chloride to afford the silyl ether, which was treated with n-BuLi in THF at low temperature with bubbled carbon dioxide to afford the carboxylic acid. This carboxylic acid was converted to acyl chloride B upon treatment with oxyalyl chloride in DMF/DCM.

Separately, 5-formylfuran-2-carboxylic acid was reacted with trimethylsilyldiazomethane in benzene/methanol, followed by reduction with sodium borohydride to afford alcohol C.

A and B were condensed using copper (I) iodide in the presence of palladium catalyst and organic base. The resulting compound was treated with methylhydrazine in ethanol, followed by deprotection with fluoride at decreased temperature in THF to afford the intermediate phenol. This was treated with intermediate C in the presence of diethyl azodicarboxylate and triphenylphosphine to afford the ether product. The methyl ester was then deprotected with lithium hydroxide in THF, followed by Boc deprotection with acid in dioxane. This was then treated with N,N′-bis-Boc-1-guanylpyrazole in the presence of organic base. The remaining Boc groups were removed with TFA/DCM. The carboxylic acid was activated using HBTU for coupling with H₂N-GN3 (acetyl groups removed with sodium methoxide in methanol) in the presence of organic base in DMF. Amide formation resulted in the final compound.

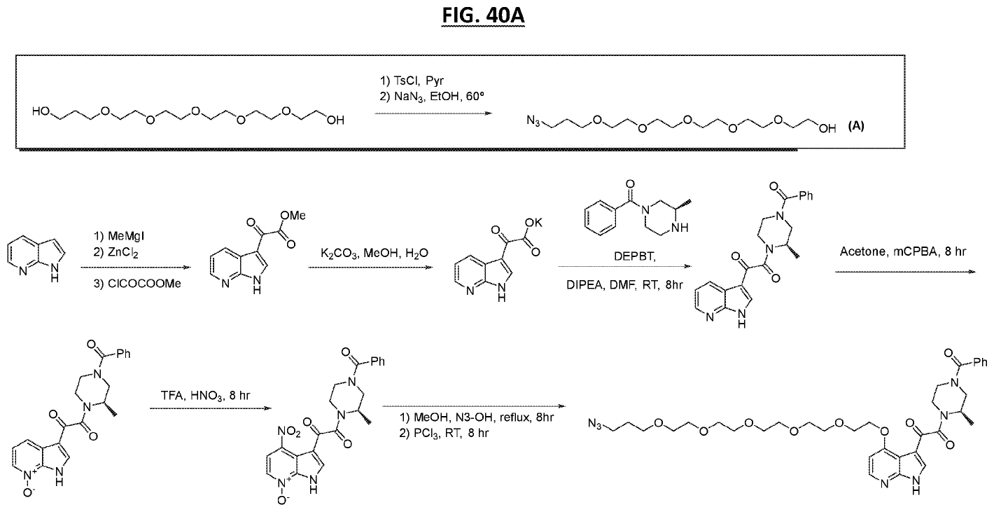

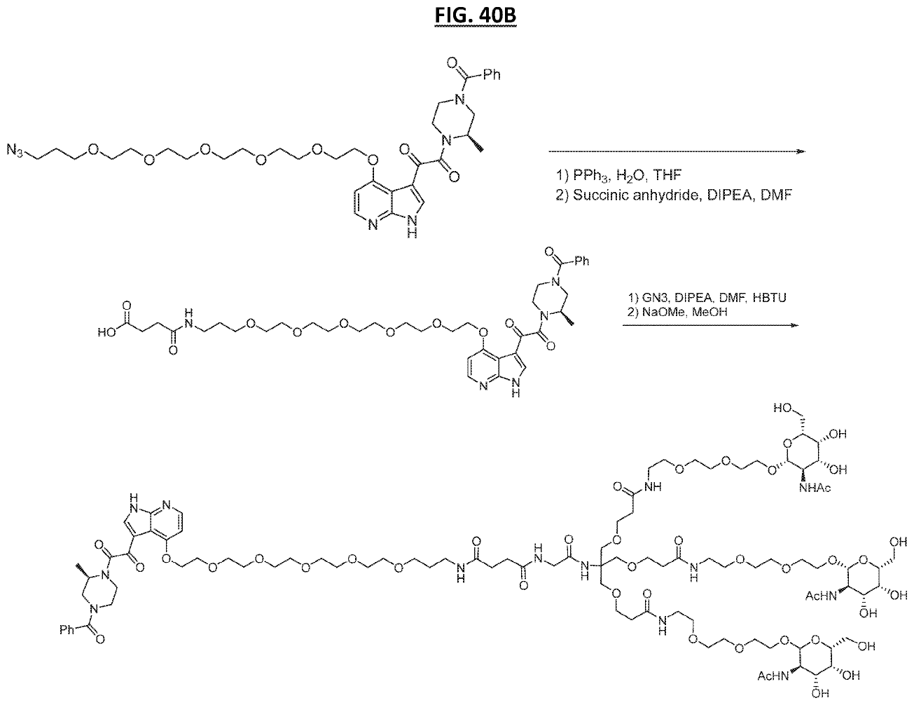

### Example 14: N1-(1-(((2R,3R,4R,5R,6R)-3-acetamido-4,5-dihydroxy-6-(hydroxymethyl)tetrahydro-2H-pyran-2-yl)oxy)-15-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-15-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-10,17-dioxo-3,6,13-trioxa-9,16-diazaoctadecan-18-yl)-N4-(1-((3-(2-((R)-4-benzoyl-2-methylpiperazin-1-yl)-2-oxoacetyl)-1H-pyrrolo[2,3-b]pyridin-4-yl)oxy)-3,6,9,12,15-pentaoxaoctadecan-18-yl)succinamide (FIG. 40B)

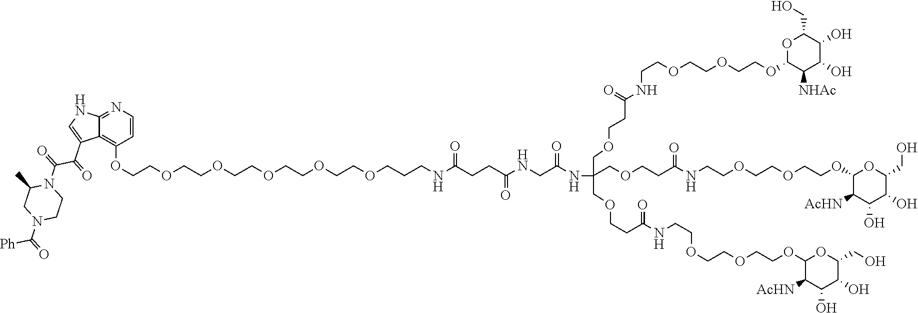 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)CCC(=O)NCCCOCCOCCOCCOCCOCCOc2ccnc3[nH]cc(C(=O)C(=O)N4CCN(C(=O)c5ccccc5)C[C@H]4C)c23)O[C@H](CO)[C@H](O)[C@@H]1O]

Hexapropylene glycol was treated with tosyl chloride in the presence of organic base, followed by treatment with sodium azide at elevated temperature to give the mono-azido alcohol A (FIG. 40A).

Separately, 1H-pyrrolo[2,3-b]pyridine was treated with methyl magnesium iodide followed by zinc (II) chloride and subsequently ClCOCOOMe to give the methyl ester derivative. Treatment with potassium bicarbonate deprotected the methyl ester to give the carboxylic acid, which was subjected to amide coupling with N-benzoyl-3-(R)-methylpiperazine in the presence of DEPBT and DIPEA in DMF. Treatment with mCPBA in acetone gave a zwitterionic intermediate, which upon treatment with nitric acid and TFA gave a mononitrated product. Treatment with intermediate A followed by reaction with PCl₃ gave the azido derivative (FIG. 40A). This was reduced using triphenyl phosphine in THF and water, followed by treatment with succinic anhydride in the presence of organic base to give the carboxylic acid. This was then activated as above for coupling to H₂N-GN3I1 to give the final compound (FIG. 40B).

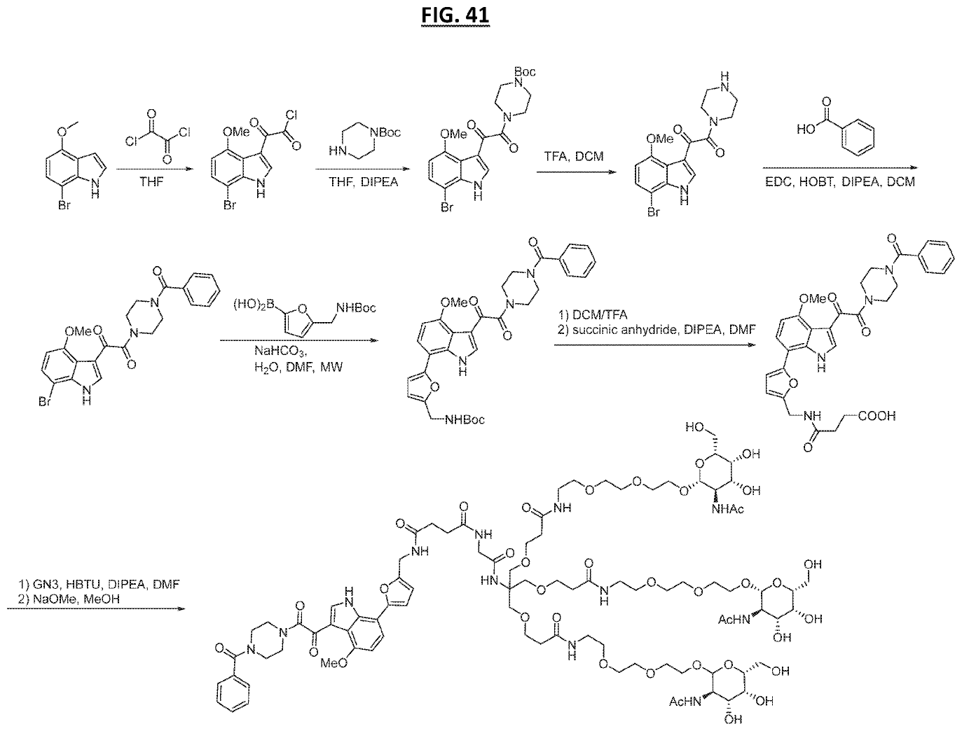

### Example 15: N1-(1-(((2R,3R,4R,5R,6R)-3-acetamido-4,5-dihydroxy-6-(hydroxymethyl)tetrahydro-2H-pyran-2-yl)oxy)-15-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-15-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-10,17-dioxo-3,6,13-trioxa-9,16-diazaoctadecan-18-yl)-N4-((5-(3-(2-(4-benzoylpiperazin-1-yl)-2-oxoacetyl)-4-methoxy-1H-indol-7-yl)furan-2-yl)methyl)succinamide (FIG. 41)

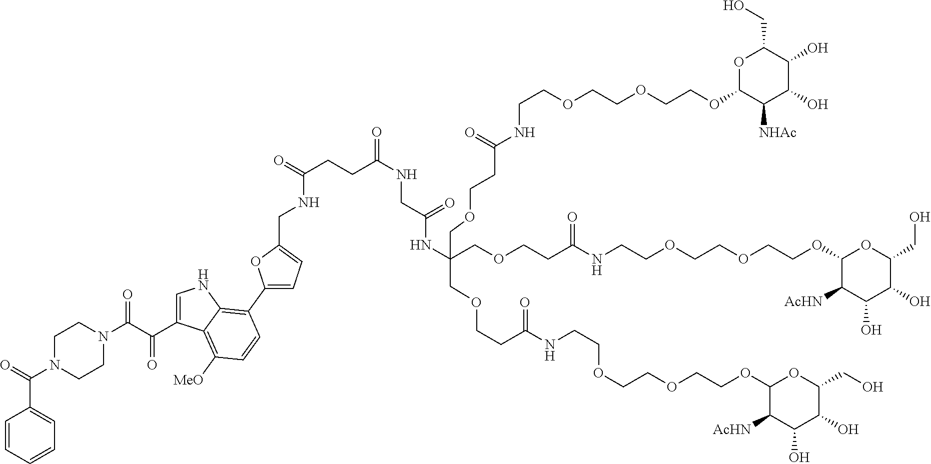 [SMILES: COc1ccc(-c2ccc(CNC(=O)CCC(=O)NCC(=O)NC(COCCC(=O)NCCOCCOCCOC3O[C@H](CO)[C@H](O)[C@H](O)[C@H]3NC(C)=O)(COCCC(=O)NCCOCCOCCO[C@@H]3O[C@H](CO)[C@H](O)[C@H](O)[C@H]3NC(C)=O)COCCC(=O)NCCOCCOCCO[C@@H]3O[C@H](CO)[C@H](O)[C@H](O)[C@H]3NC(C)=O)o2)c2[nH]cc(C(=O)C(=O)N3CCN(C(=O)c4ccccc4)CC3)c12]

7-Bromo-4-methoxy-1H-indole was treated with oxalyl chloride in THF to give the acyl chloride intermediate. Following treatment with tert-butyl piperazine-1-carboxylate in the presence of organic base, the molecule was treated with TFA in DCM to give the secondary amine intermediate. This was coupled with benzoic acid using EDC and HOBT in the presence of organic base. The bromide was condensed with (5-(((tert-butoxycarbonyl)amino)methyl)furan-2-yl)boronic acid in the presence of sodium bicarbonate in water and DMF under microwave irradiation to give the coupled intermediate, which was then deprotected (TFA in DCM) and reacted with succinic anhydride to give a carboxylic acid. The carboxylic acid was activated as above for amide coupling with H₂N-GN3 to give the final compound.

### Example 16

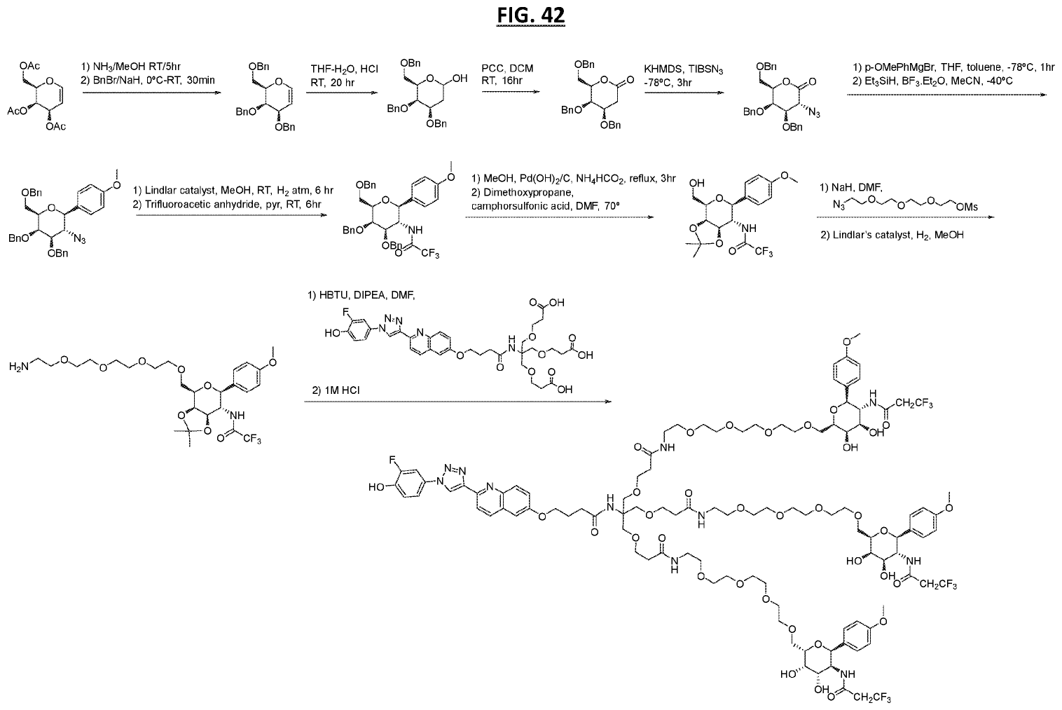

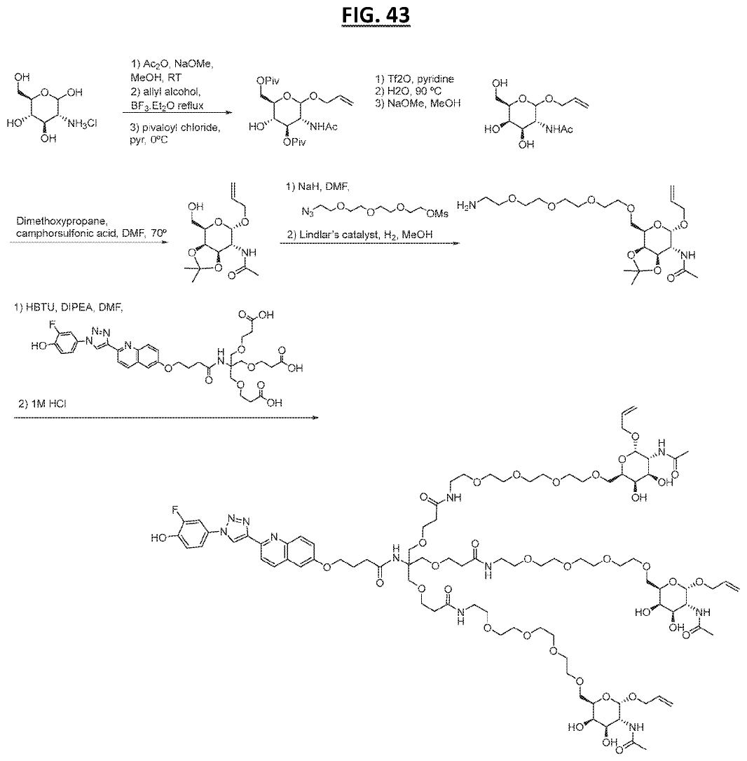

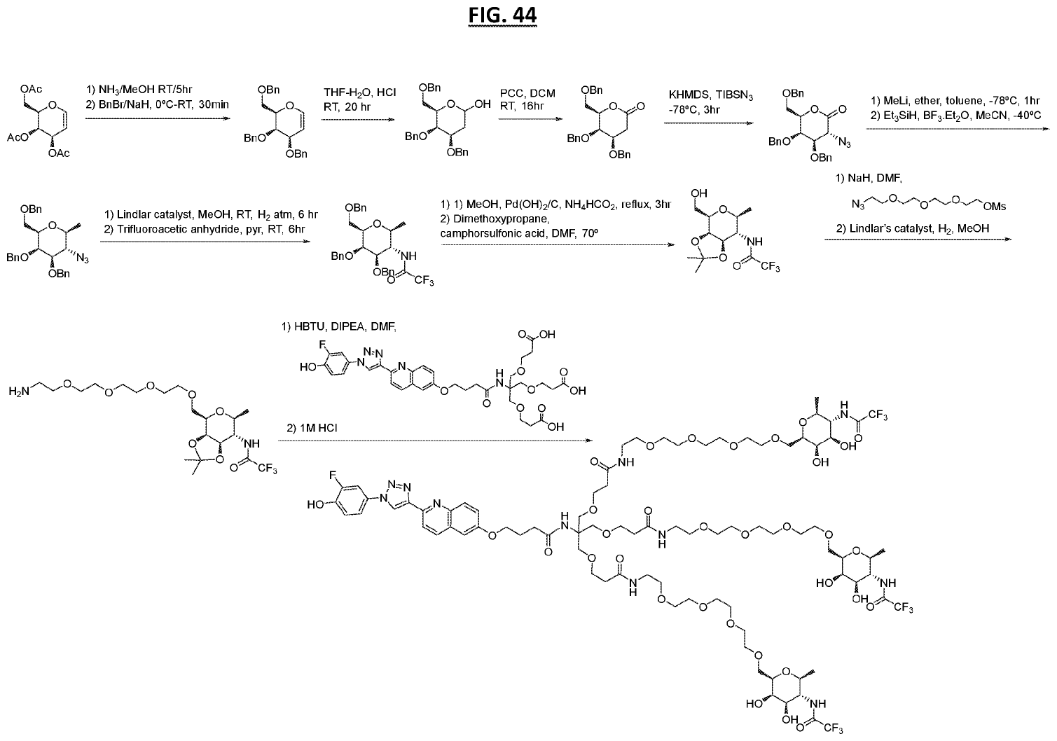

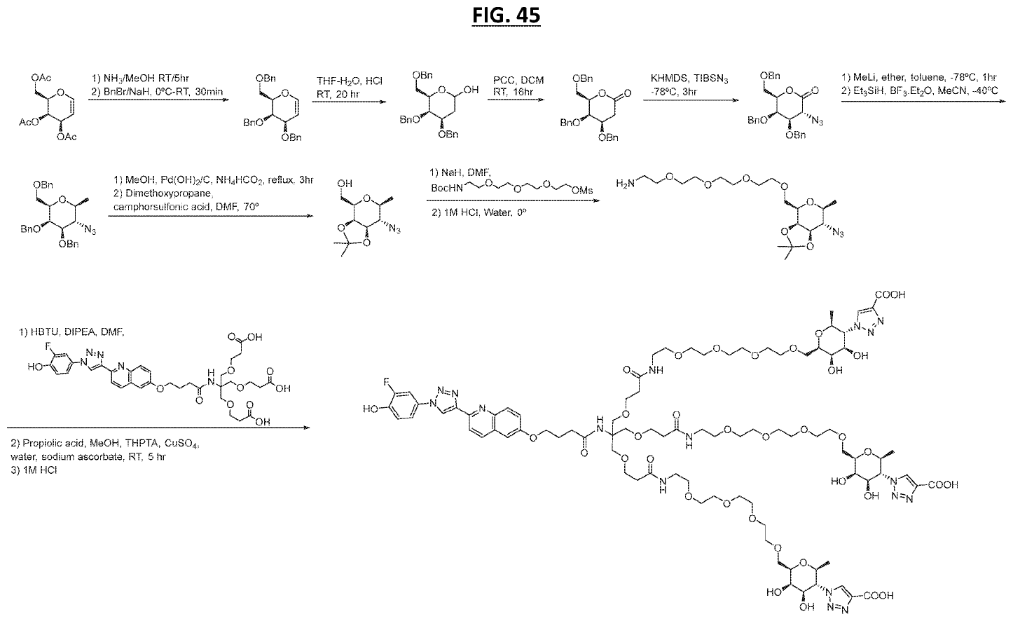

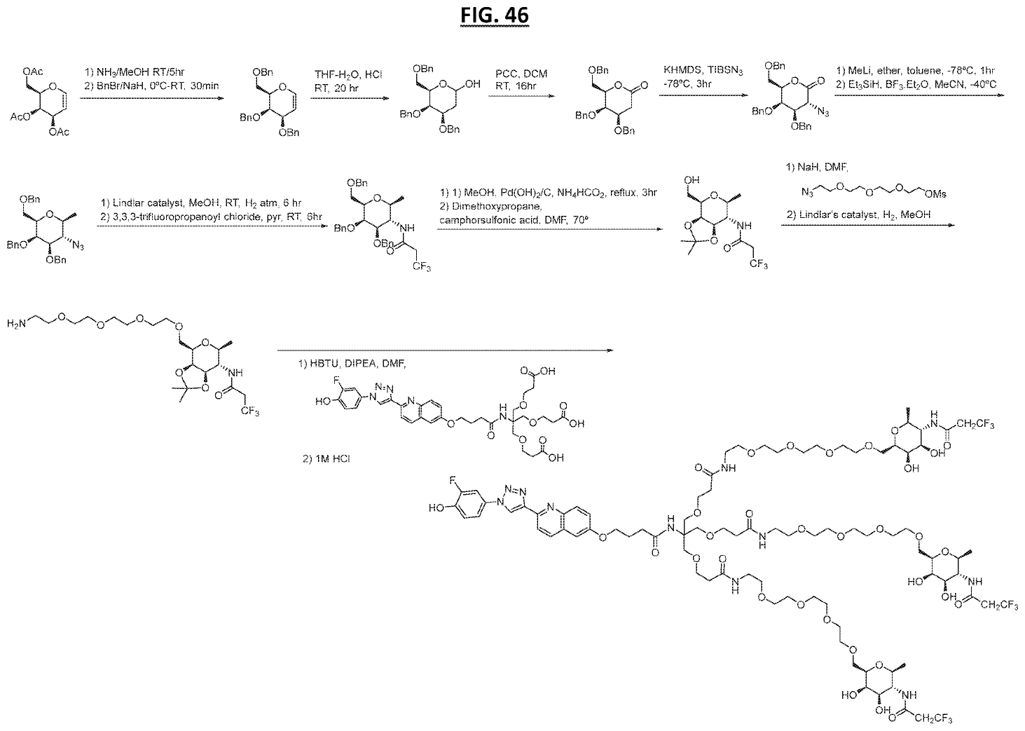

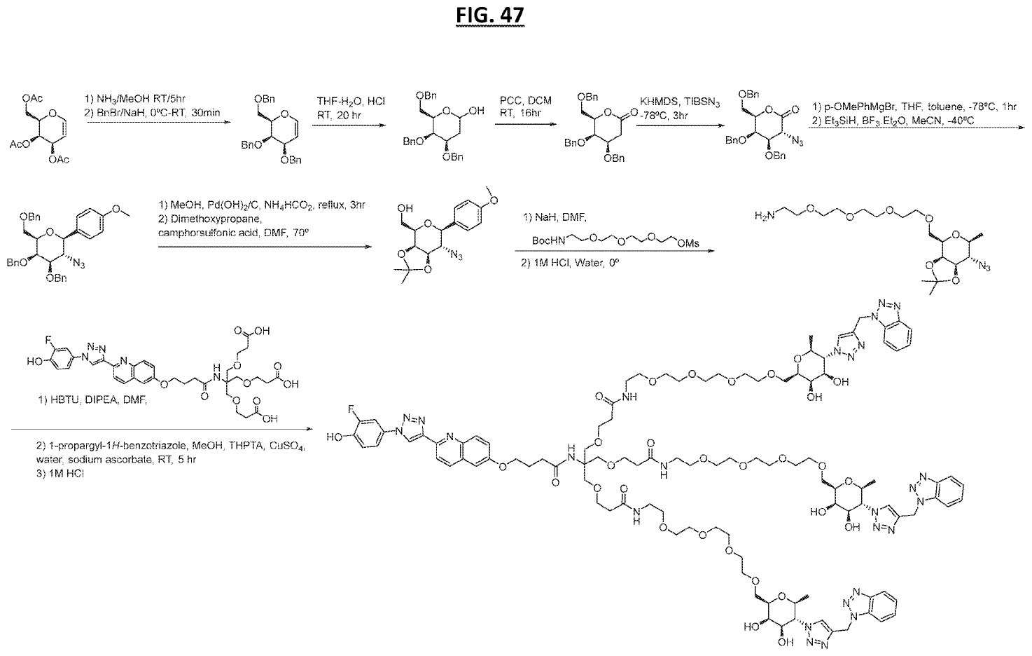

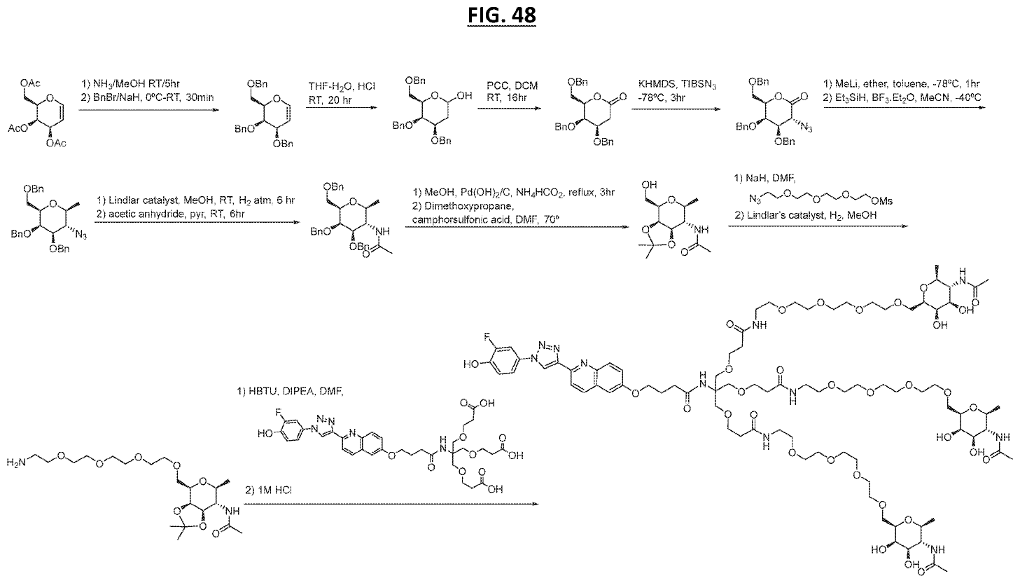

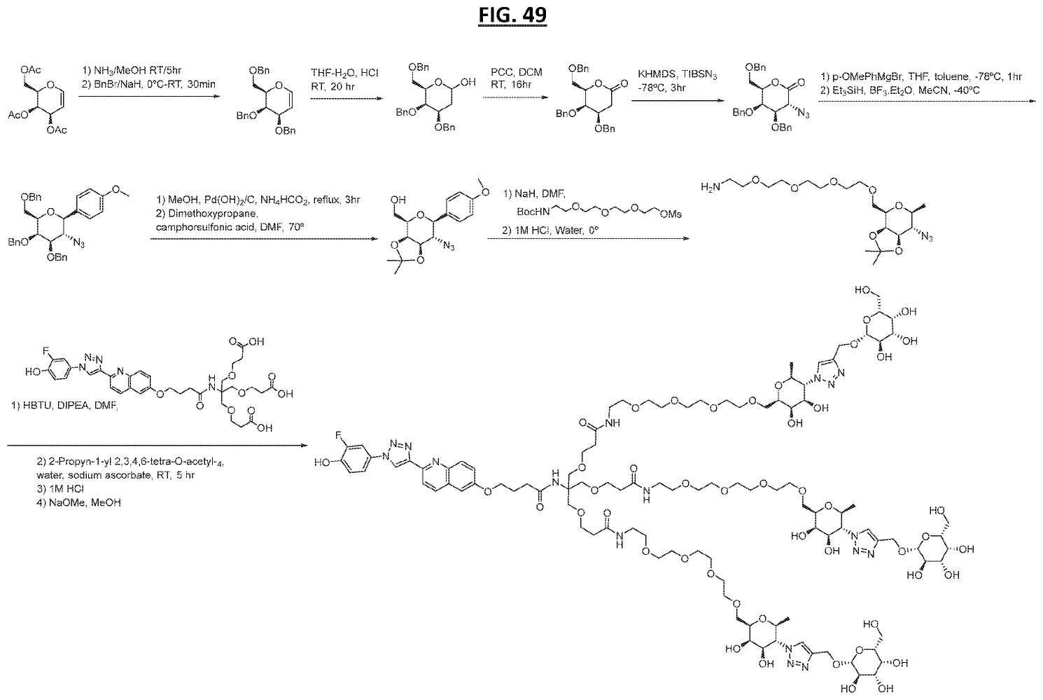

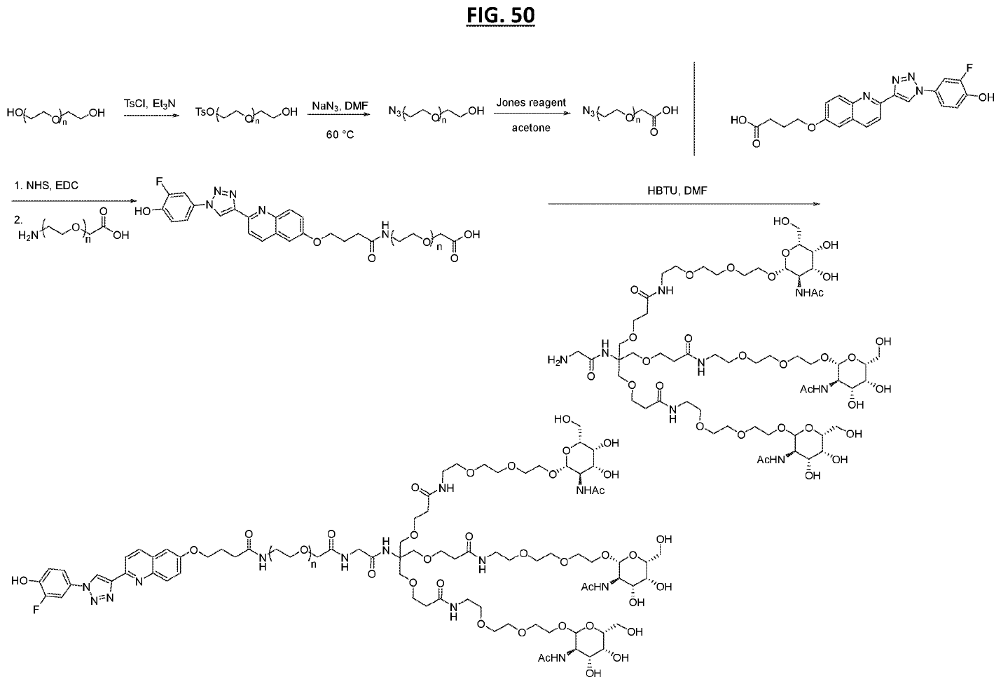

FIGS. 42-50 each illustrate the non-limiting preparation of a compound of formula (I) or formula (Ia) comprising a MIF binder.

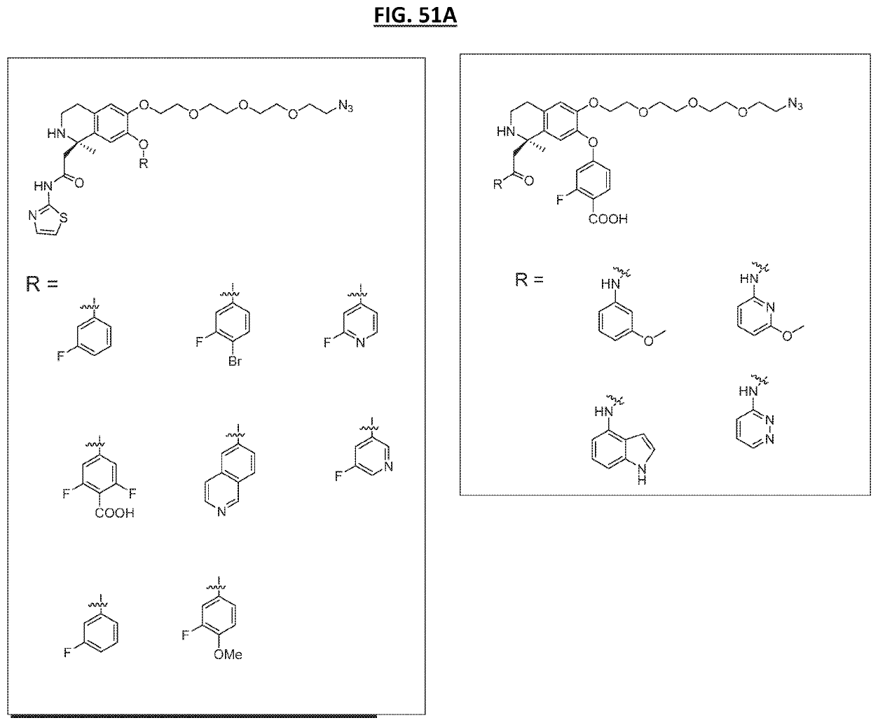

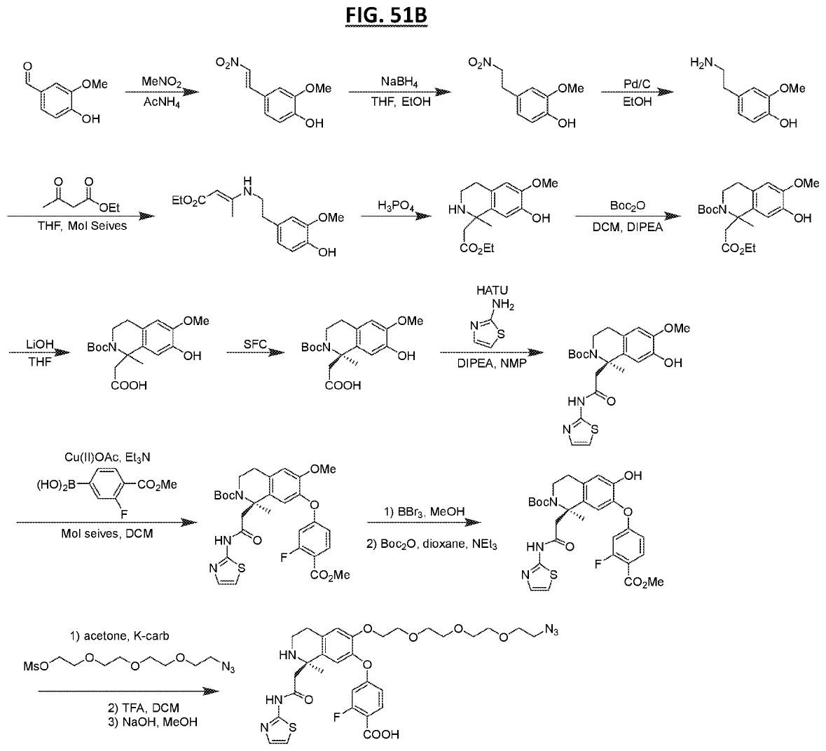

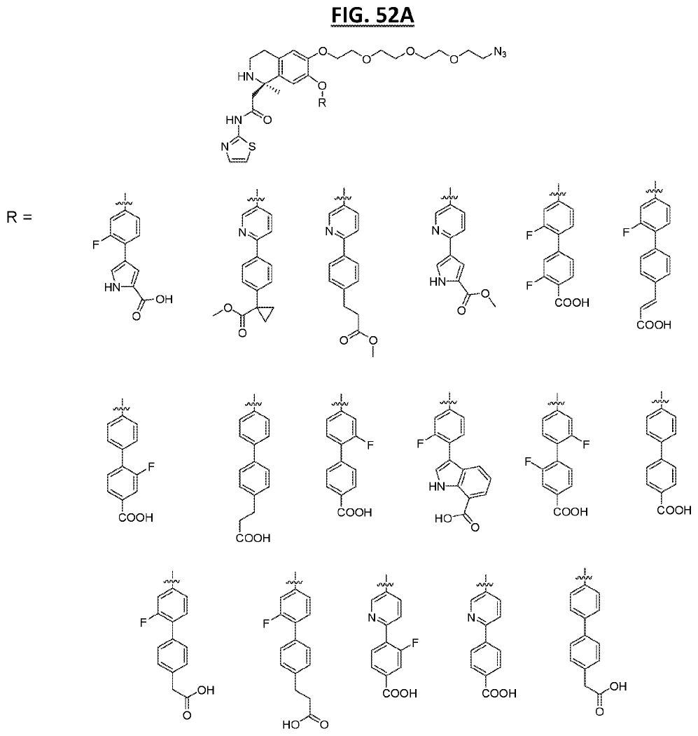

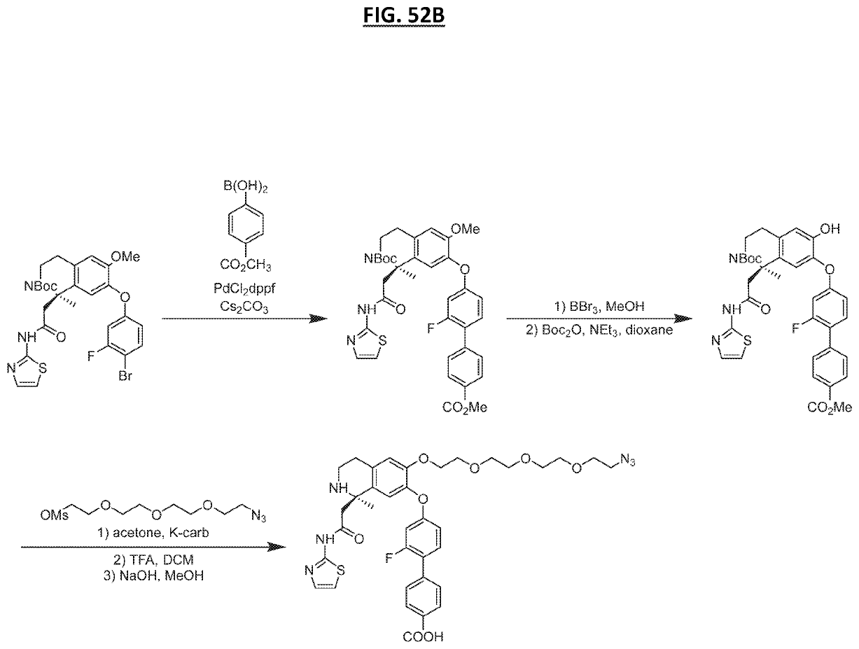

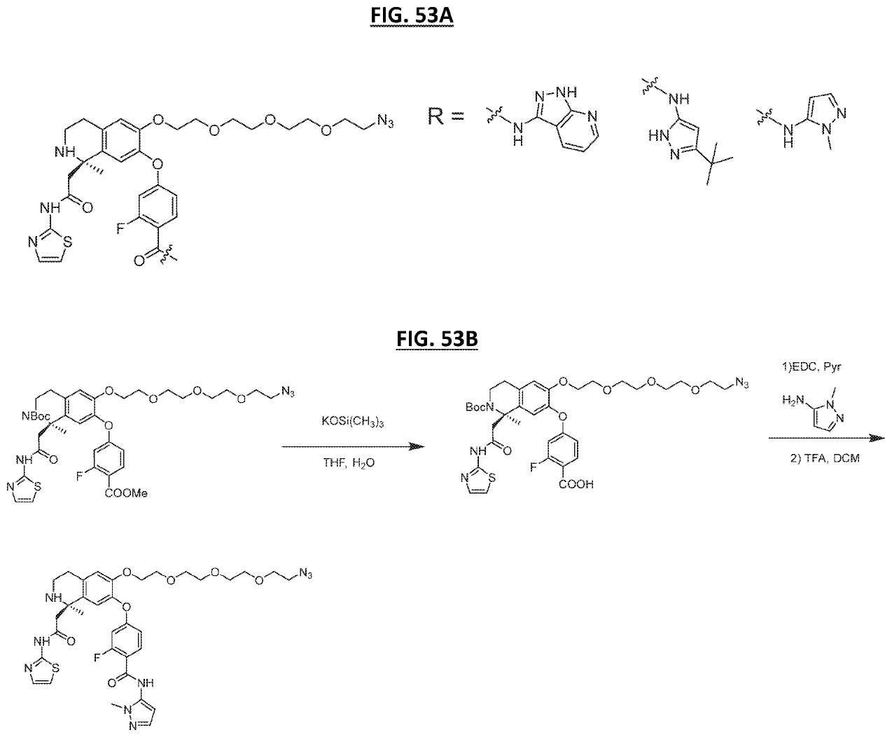

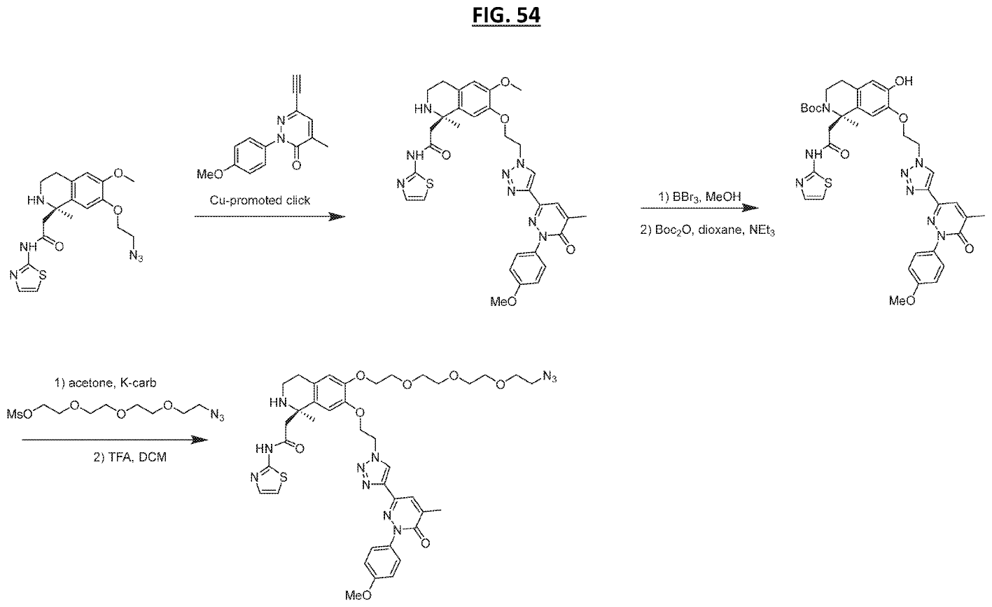

FIGS. 51A-51B, FIGS. 52A-52B, FIGS. 53A-53B, and FIG. 54 each illustrate non-limiting PCSK9 ligands which can be used in a compound of formula (I) or formula (Ia) and the illustrative synthesis thereof.

### Example 17

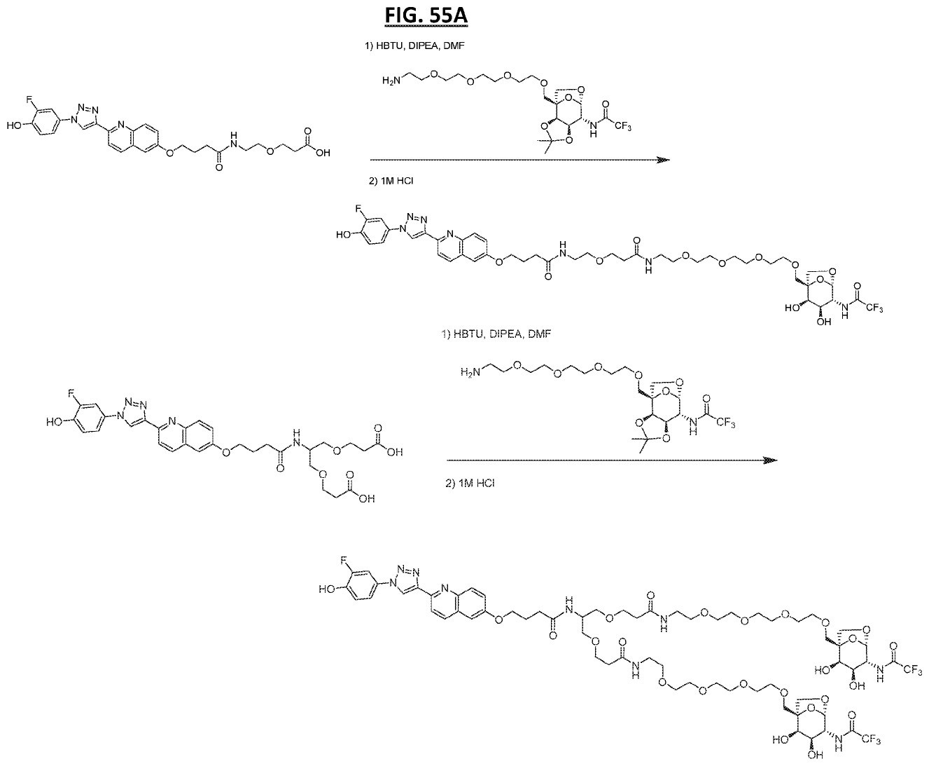

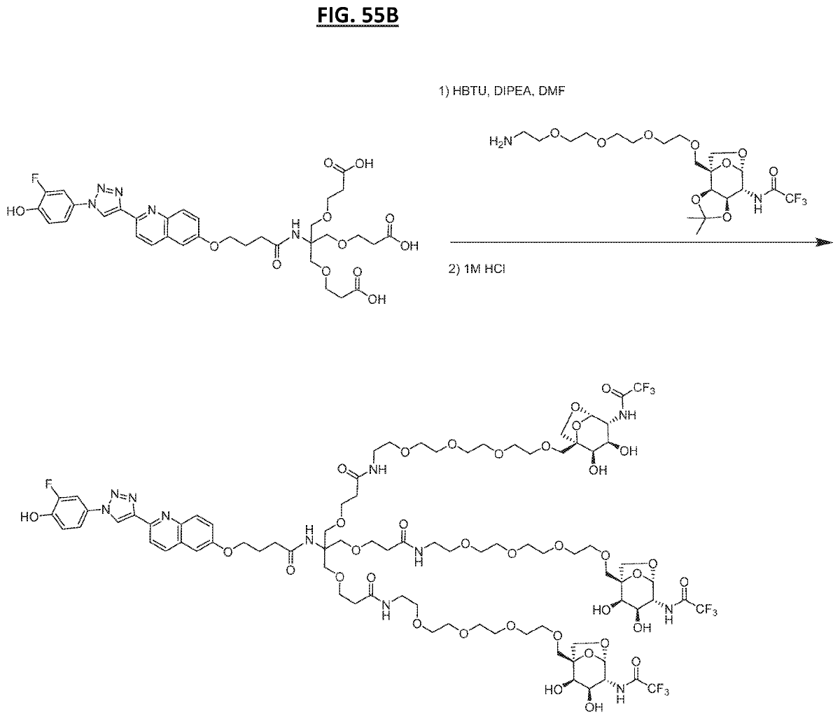

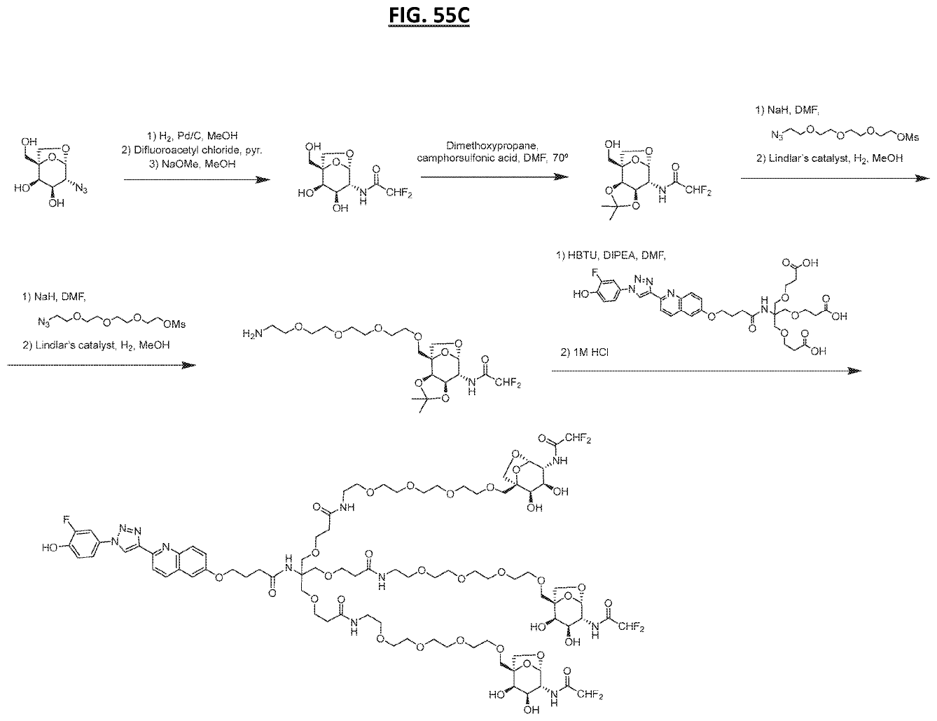

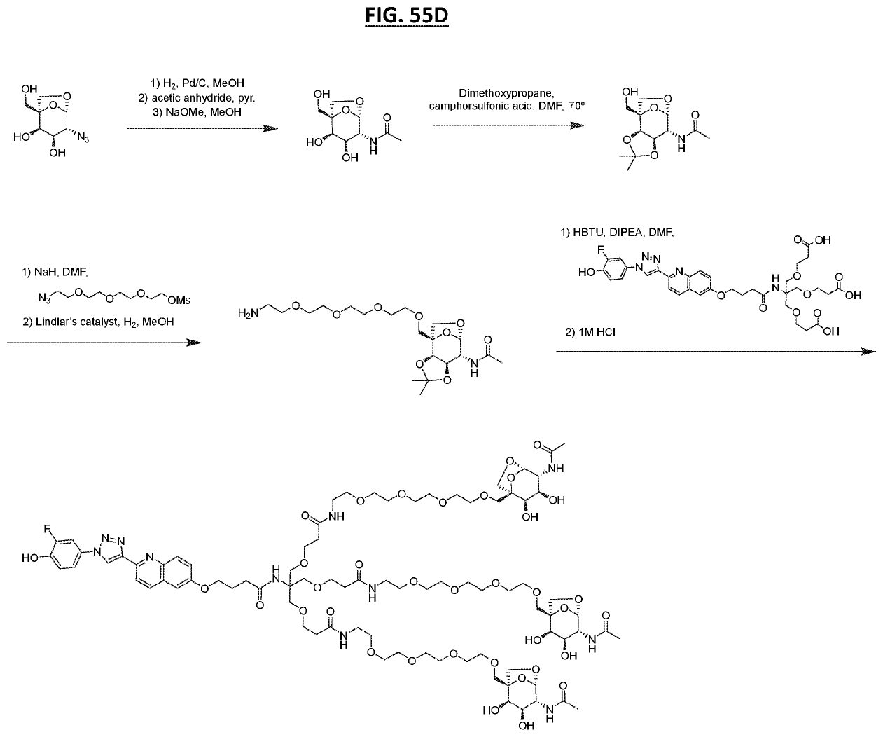

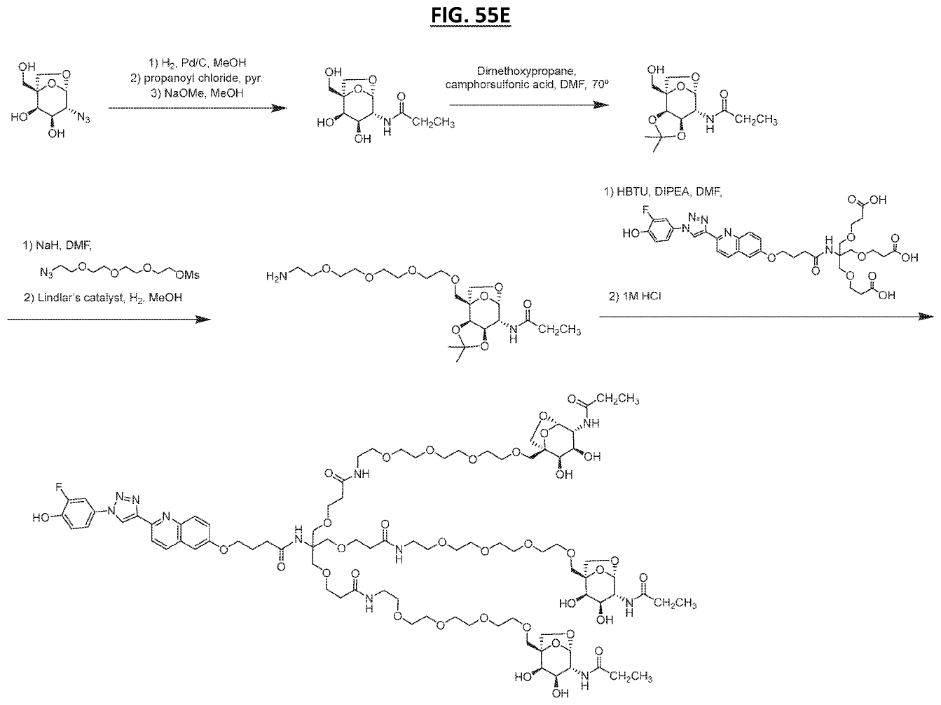

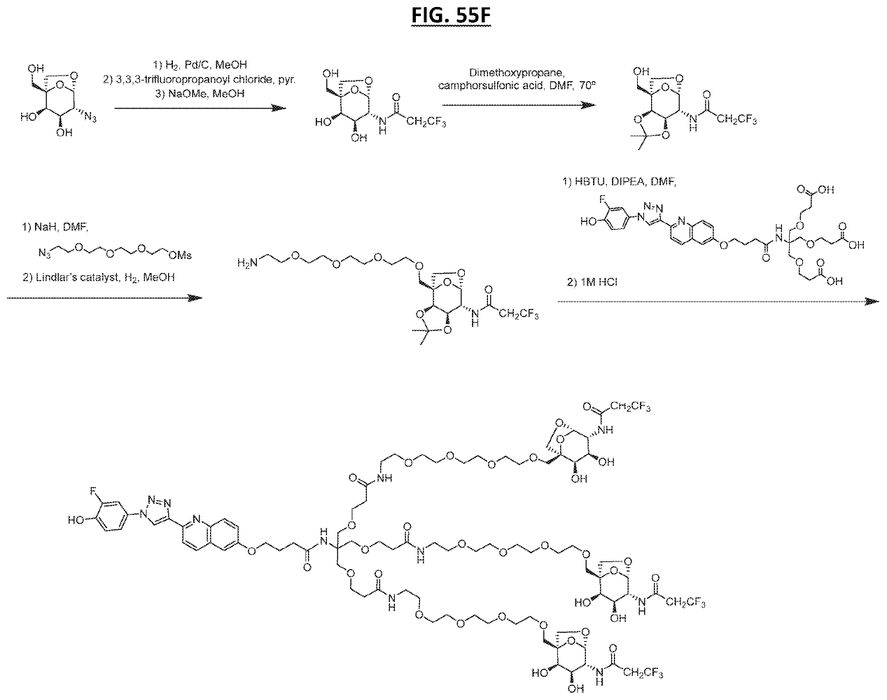

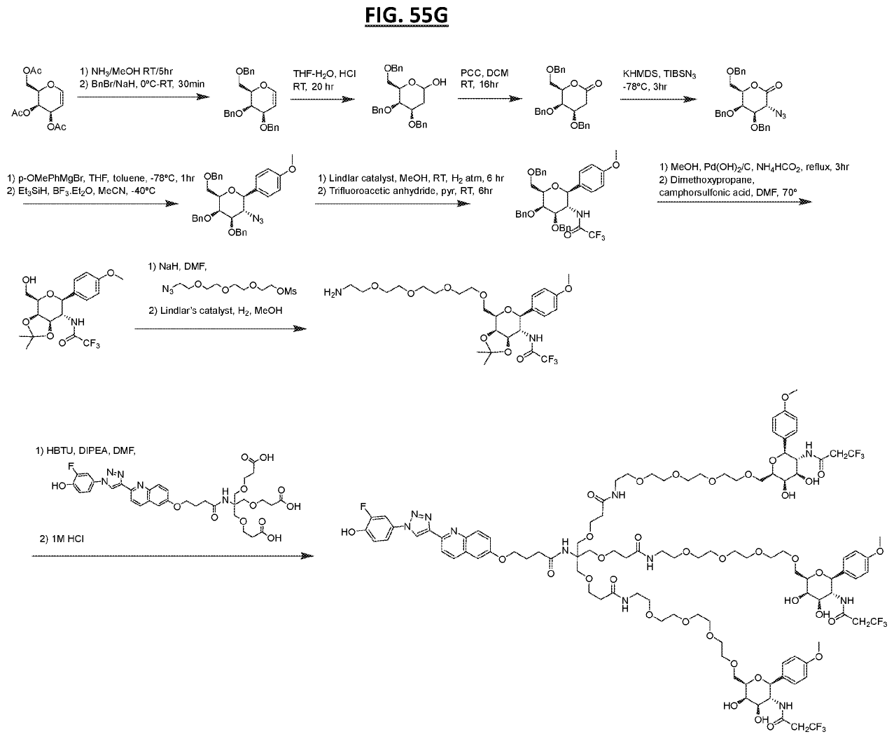

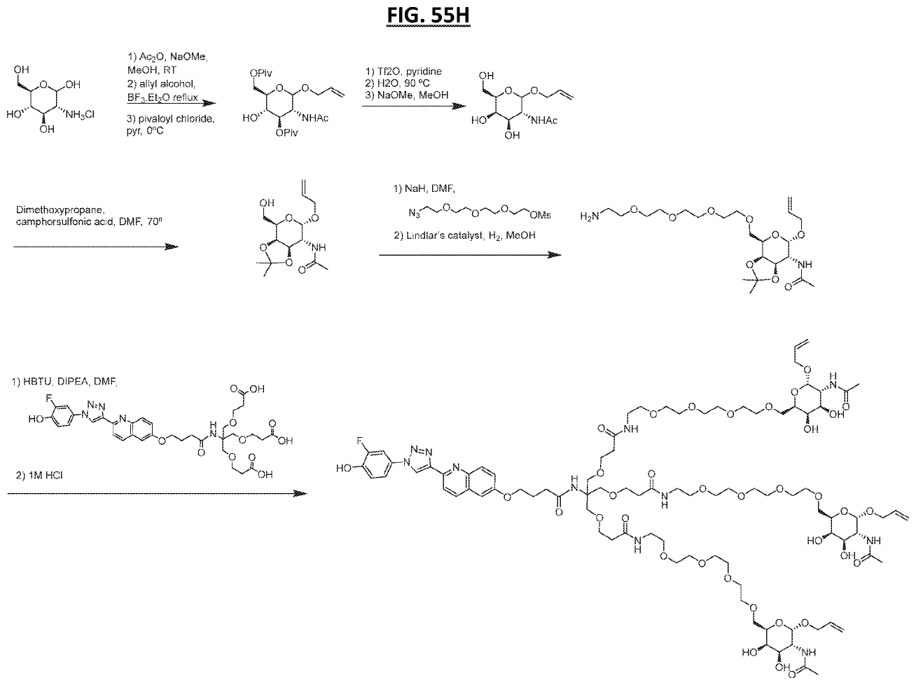

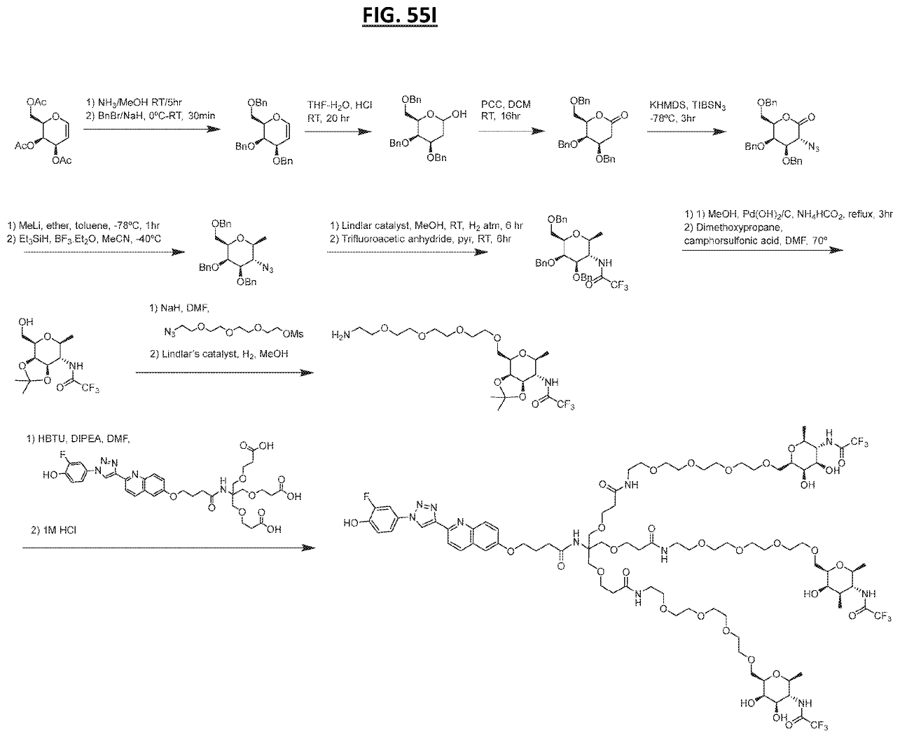

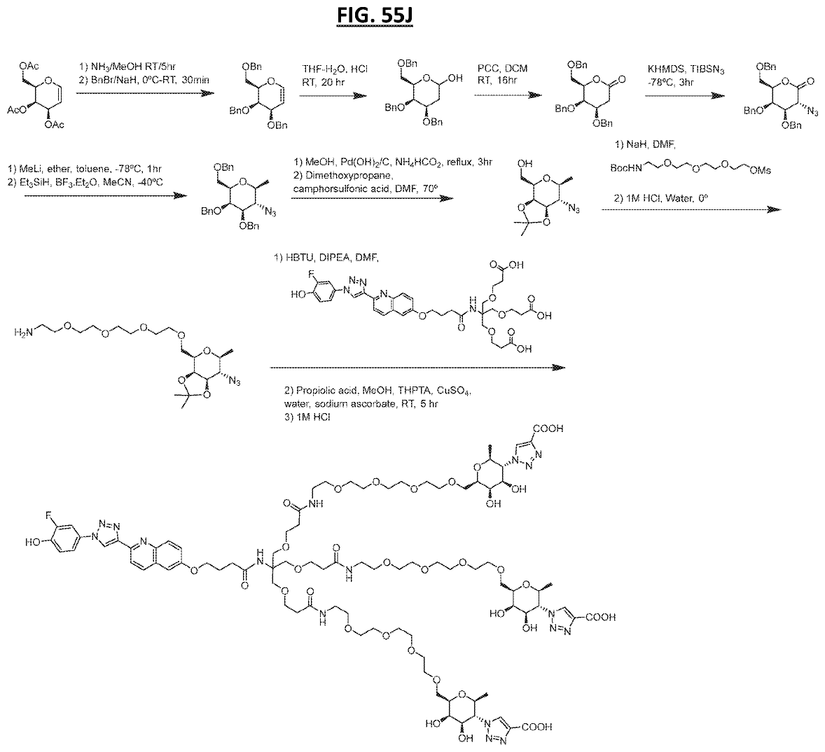

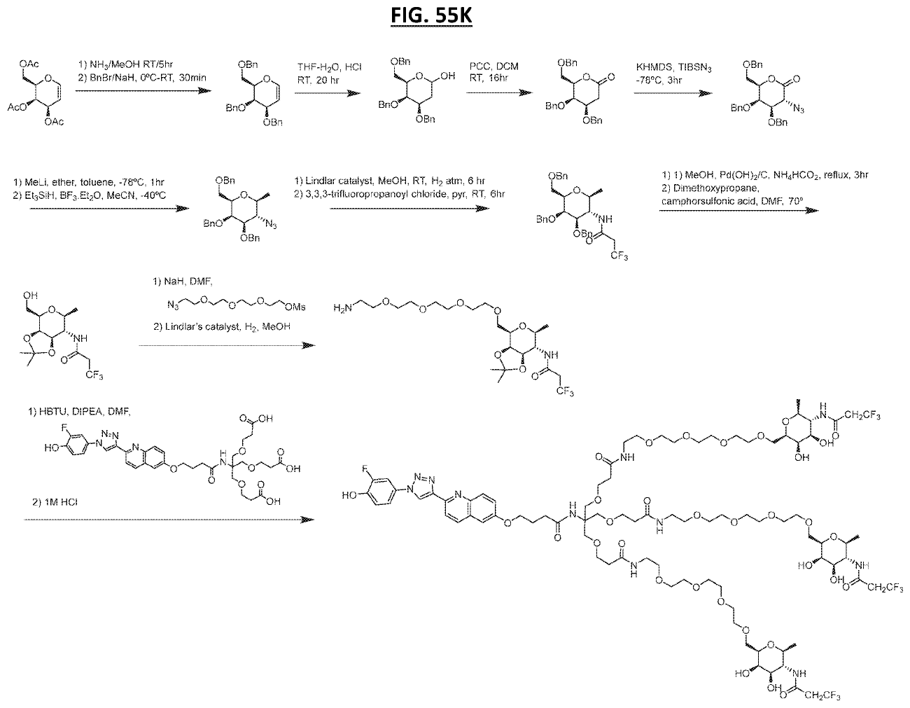

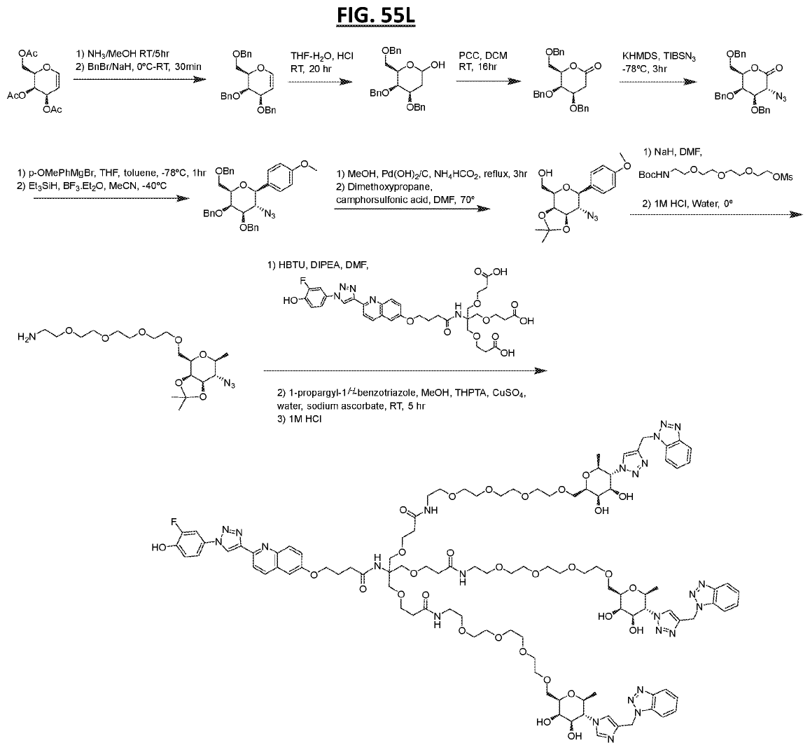

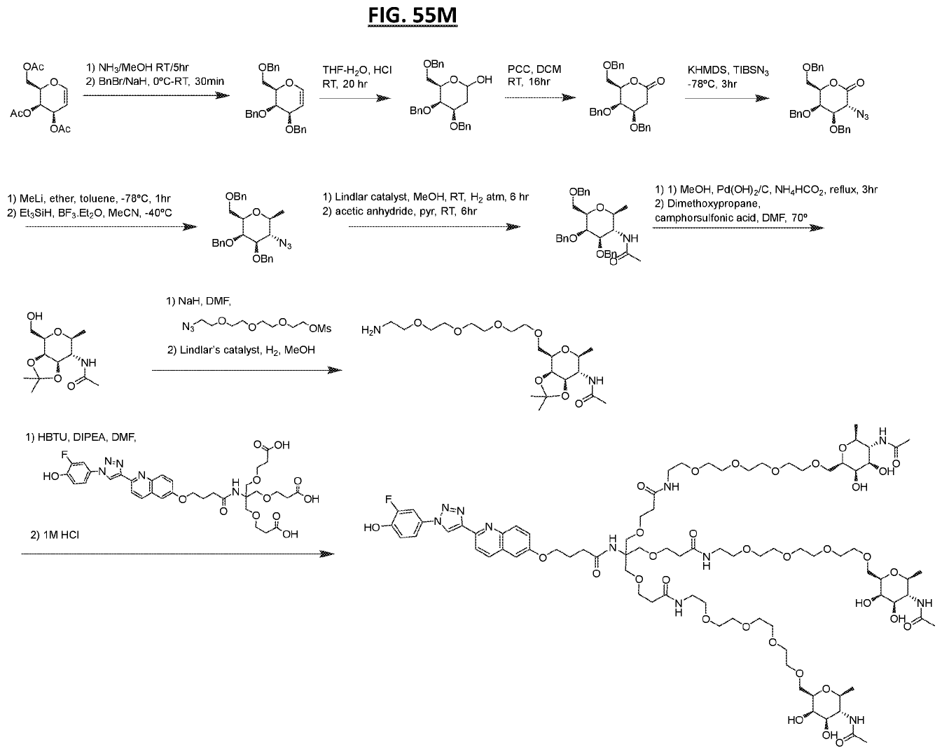

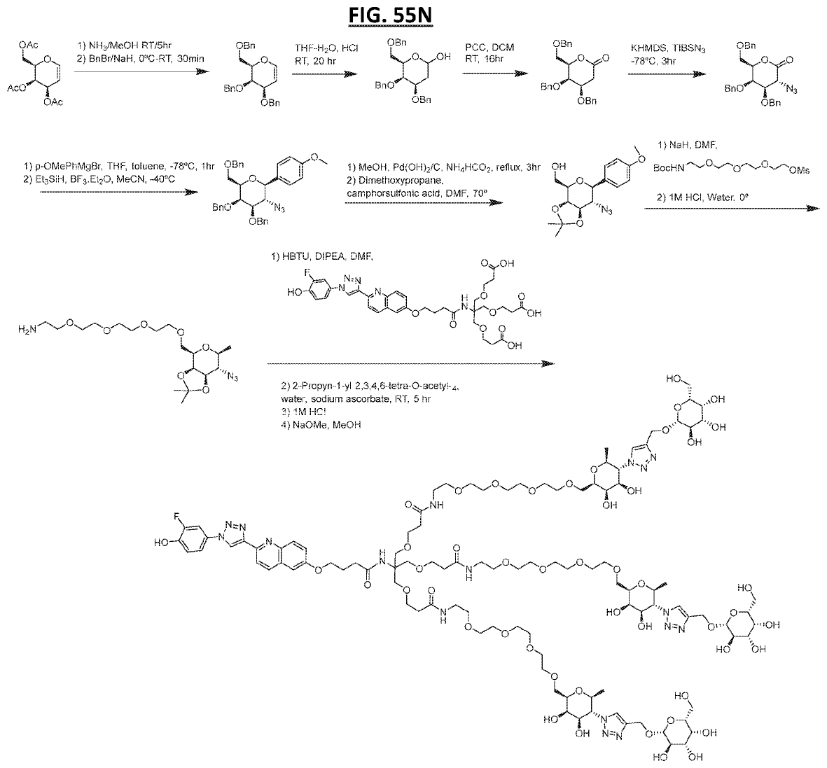

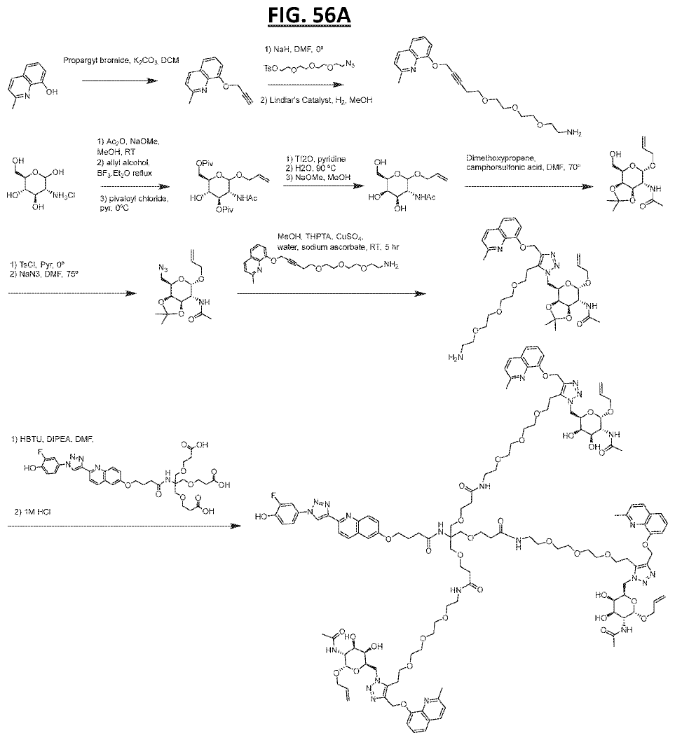

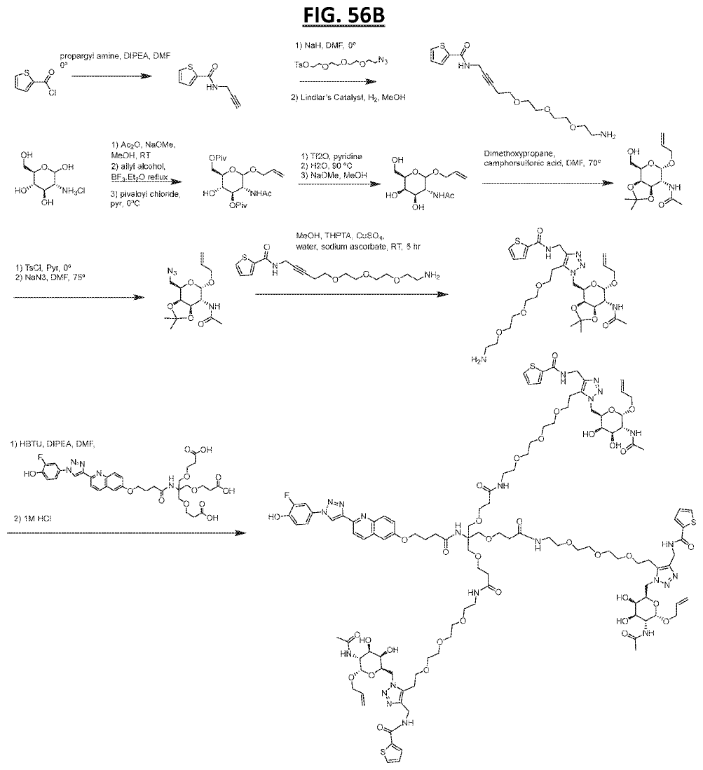

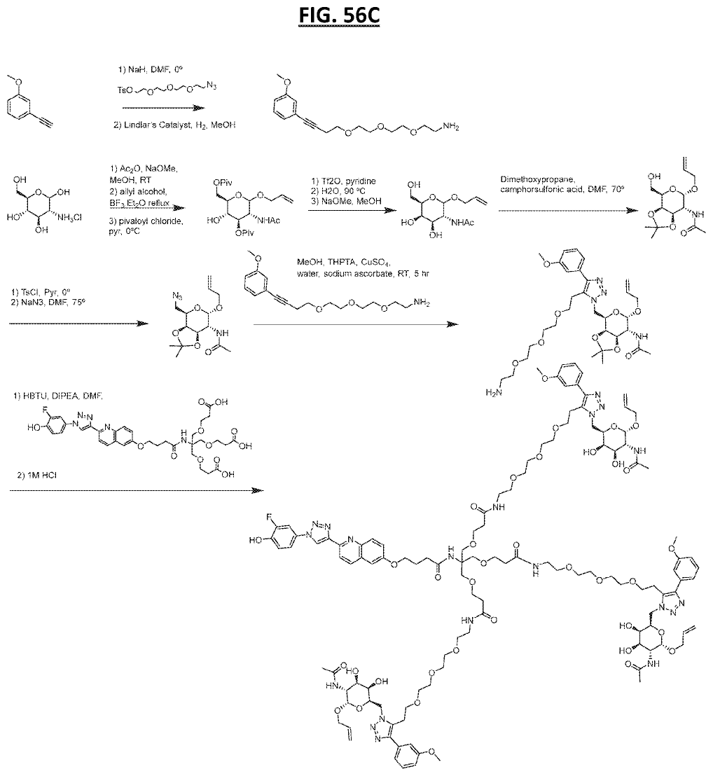

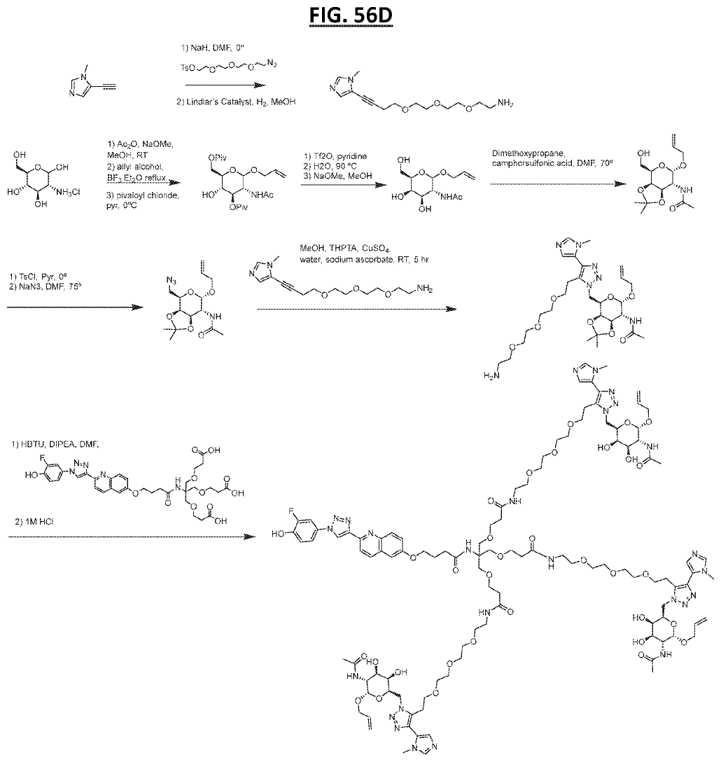

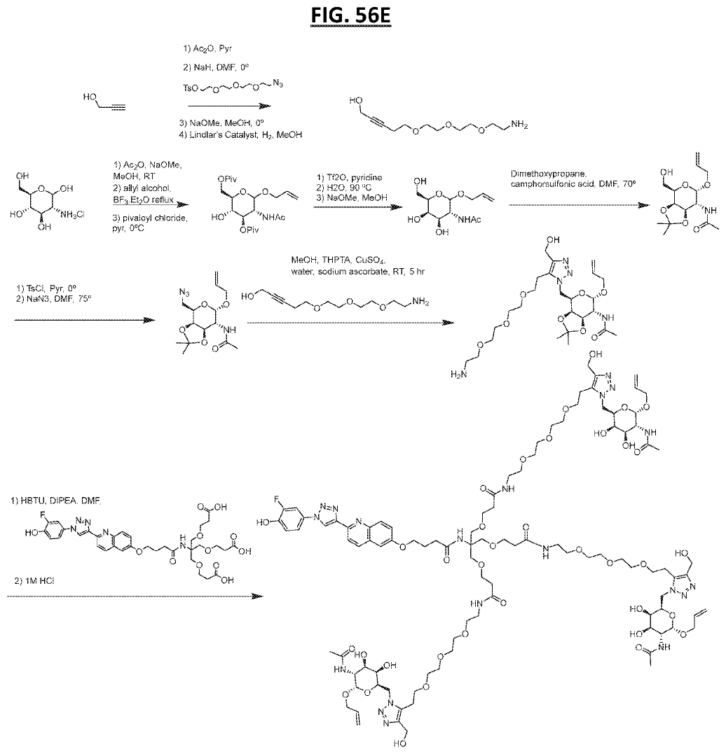

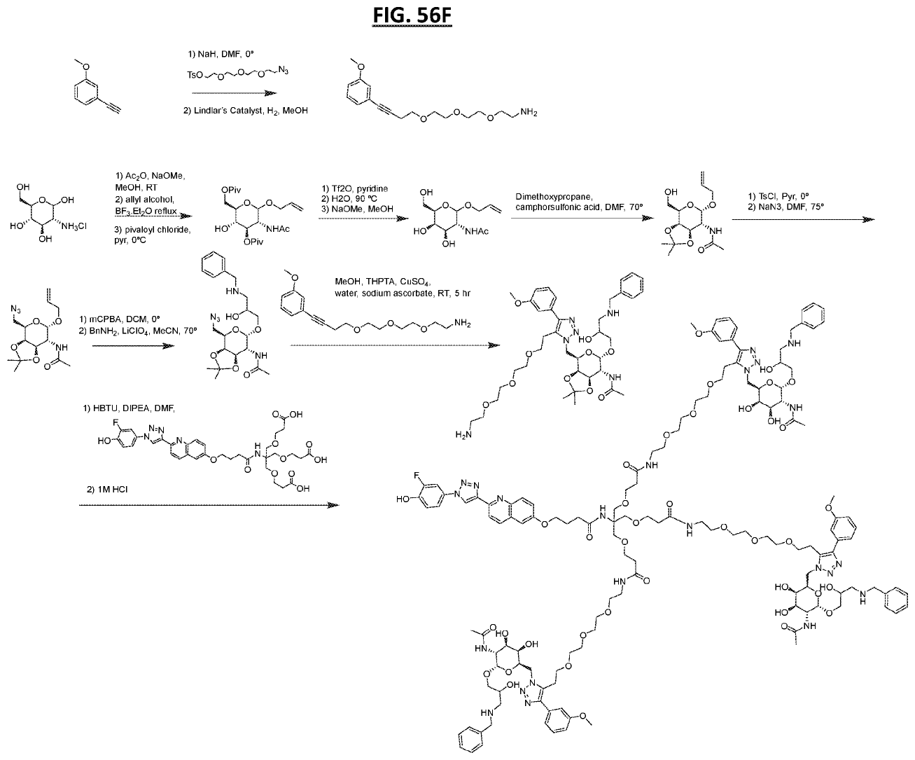

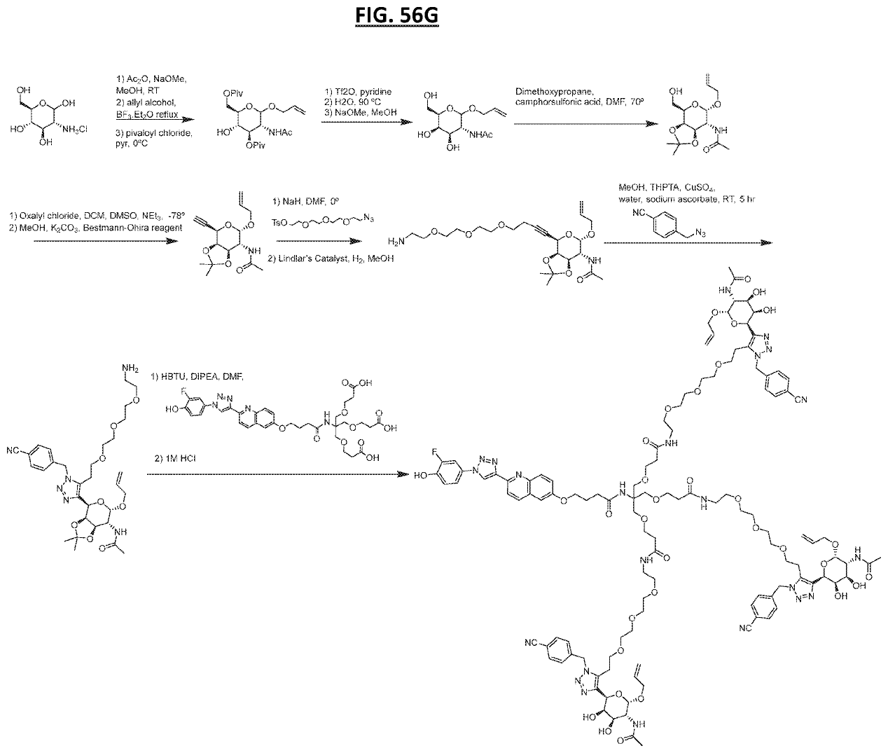

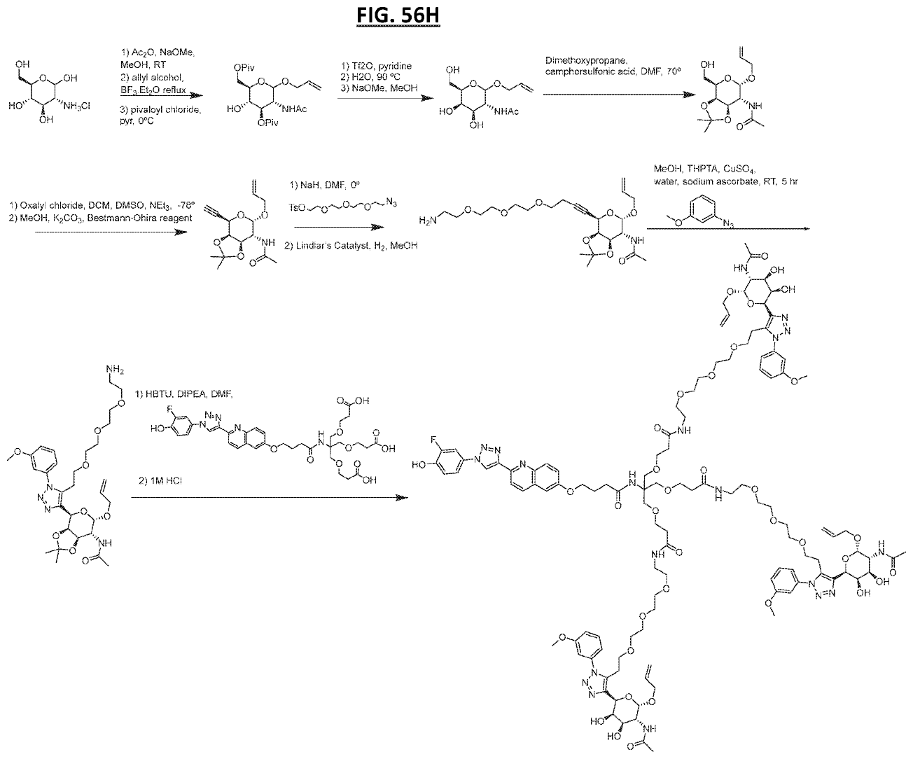

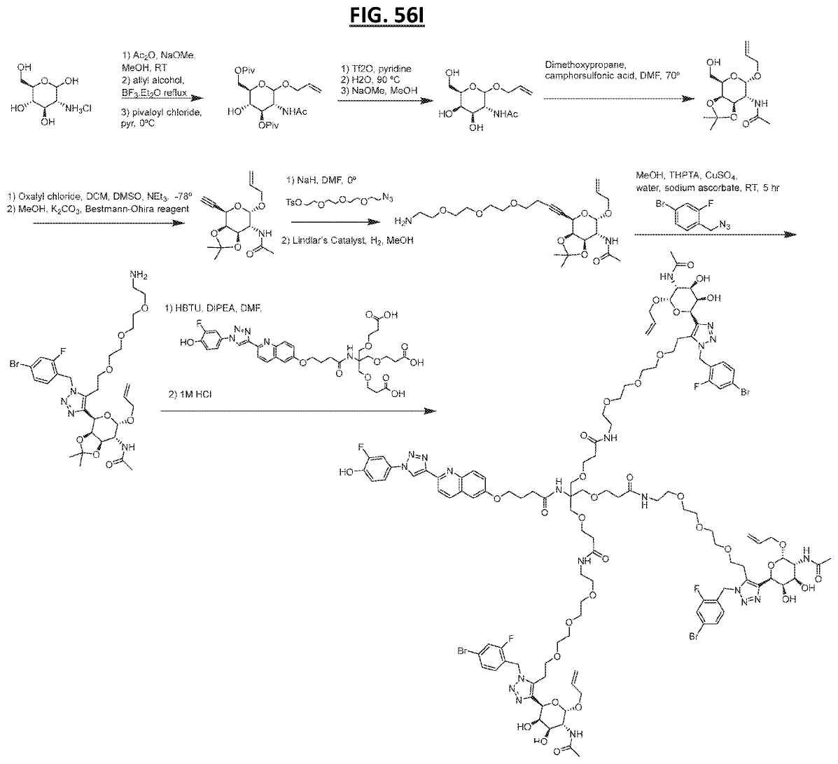

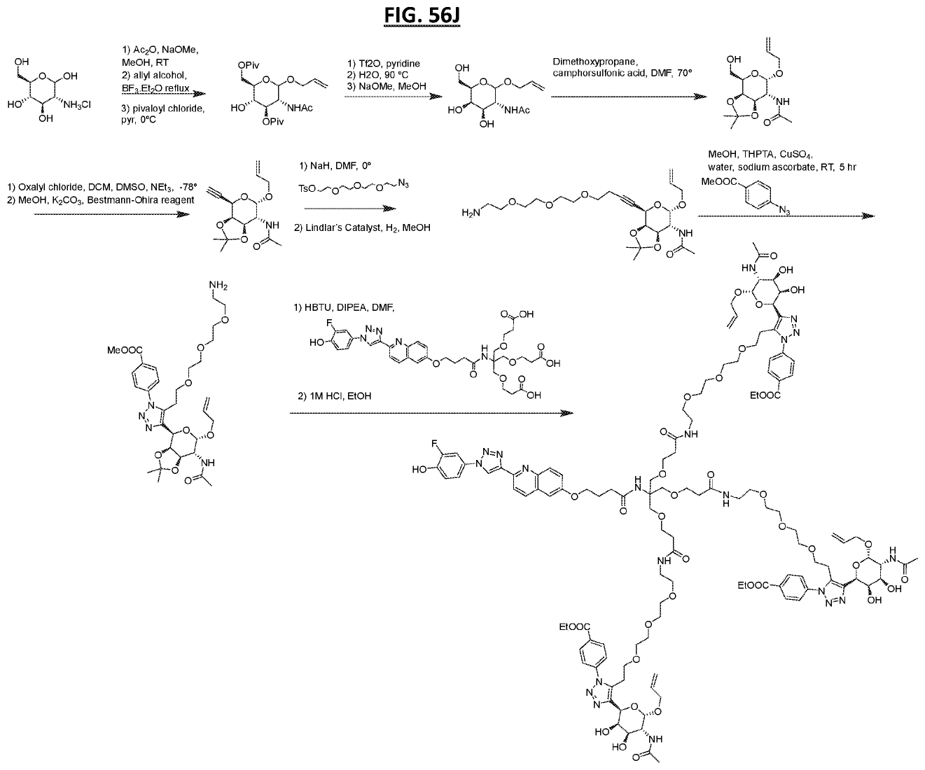

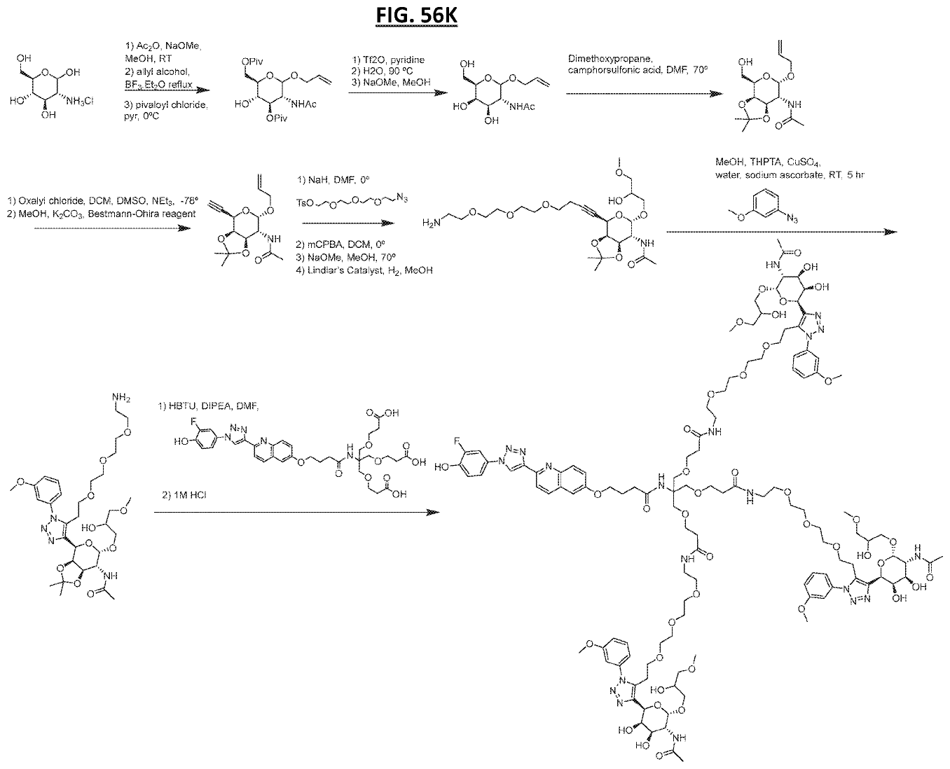

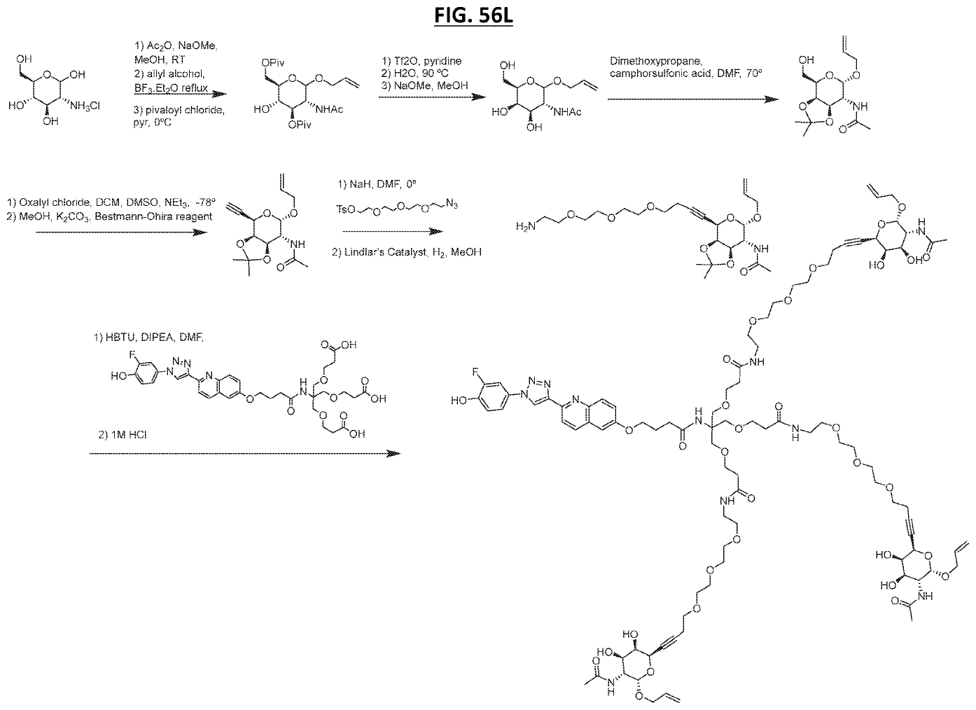

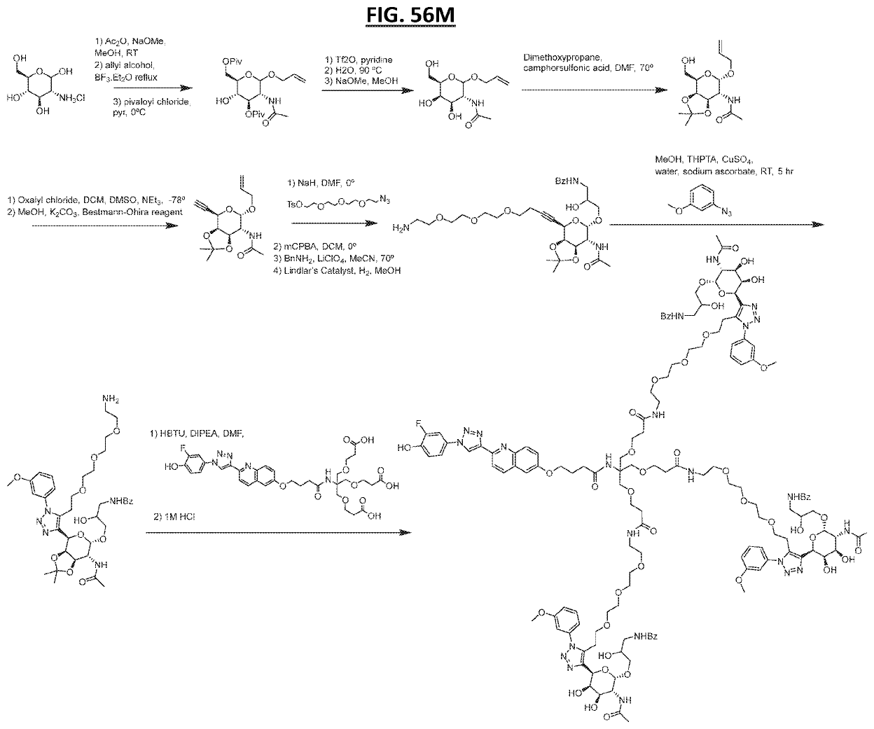

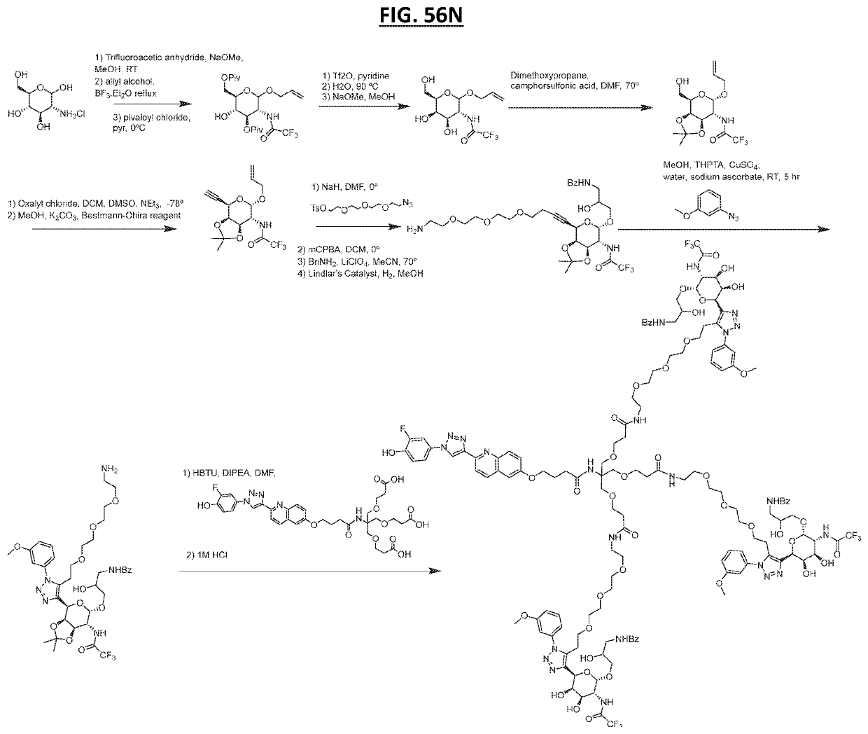

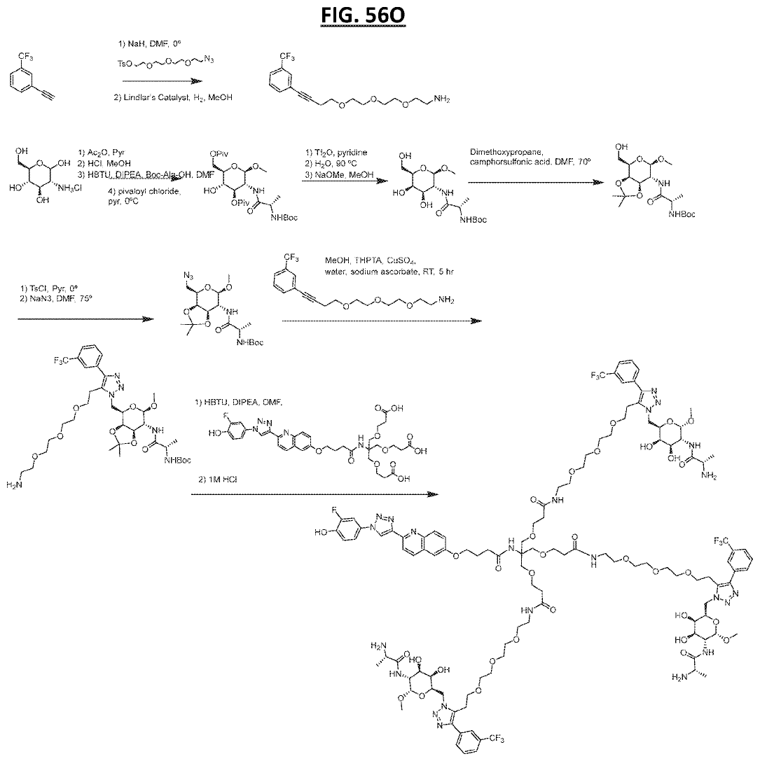

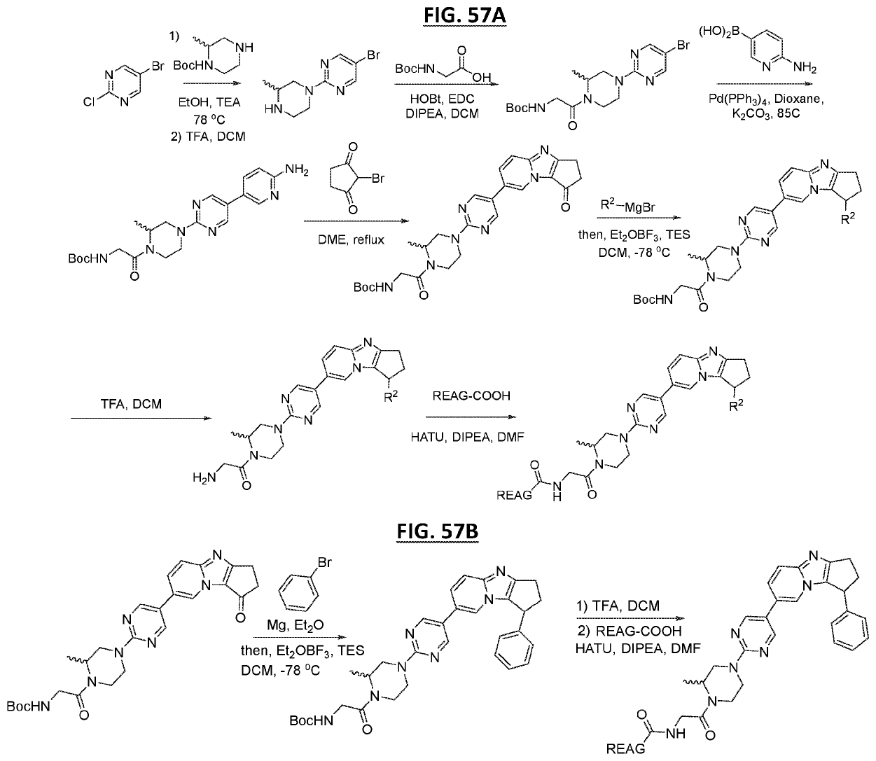

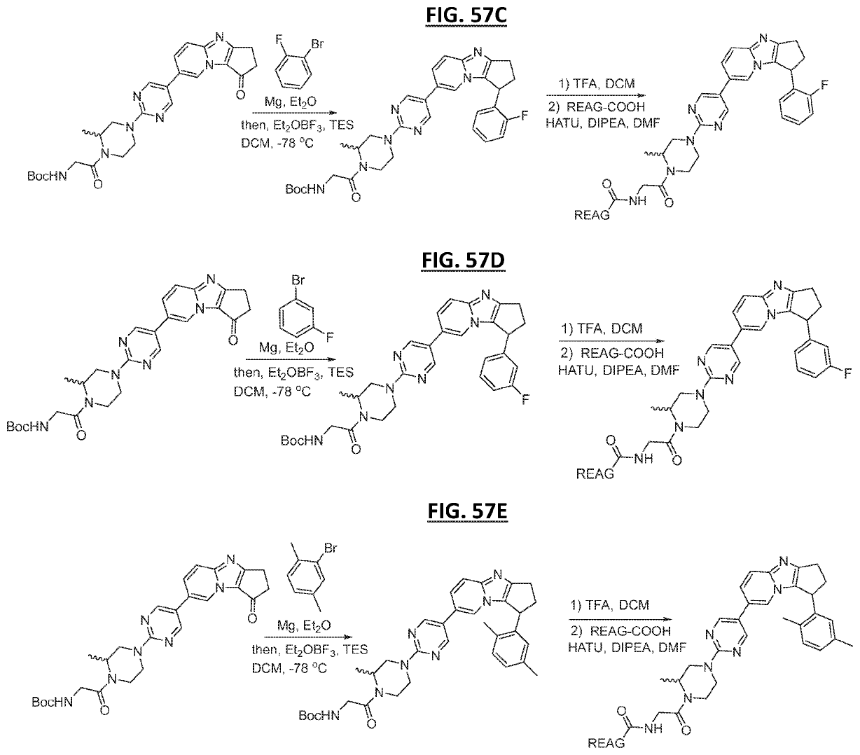

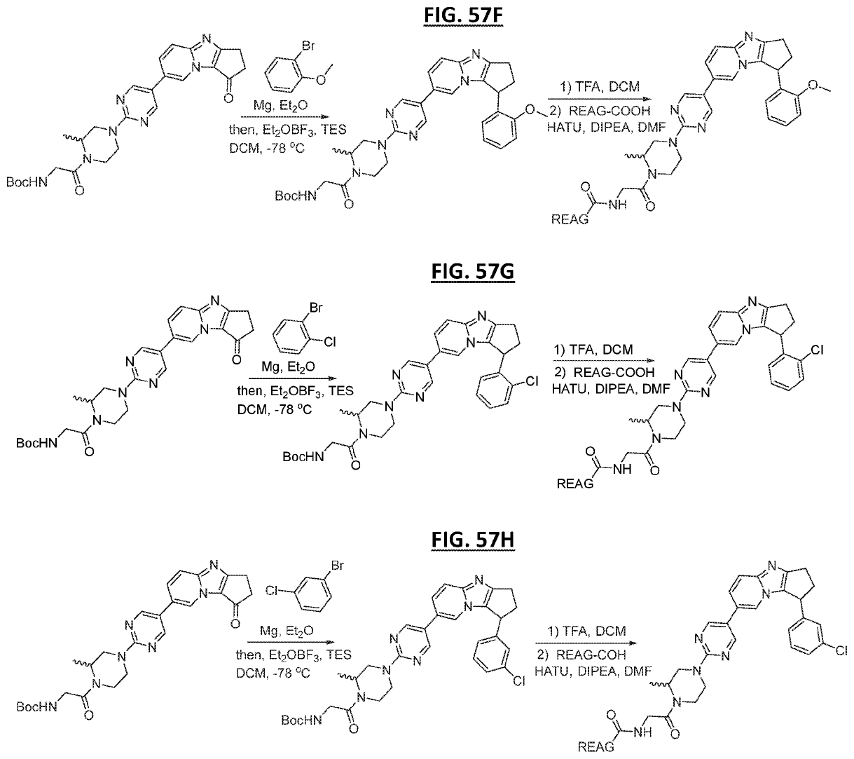

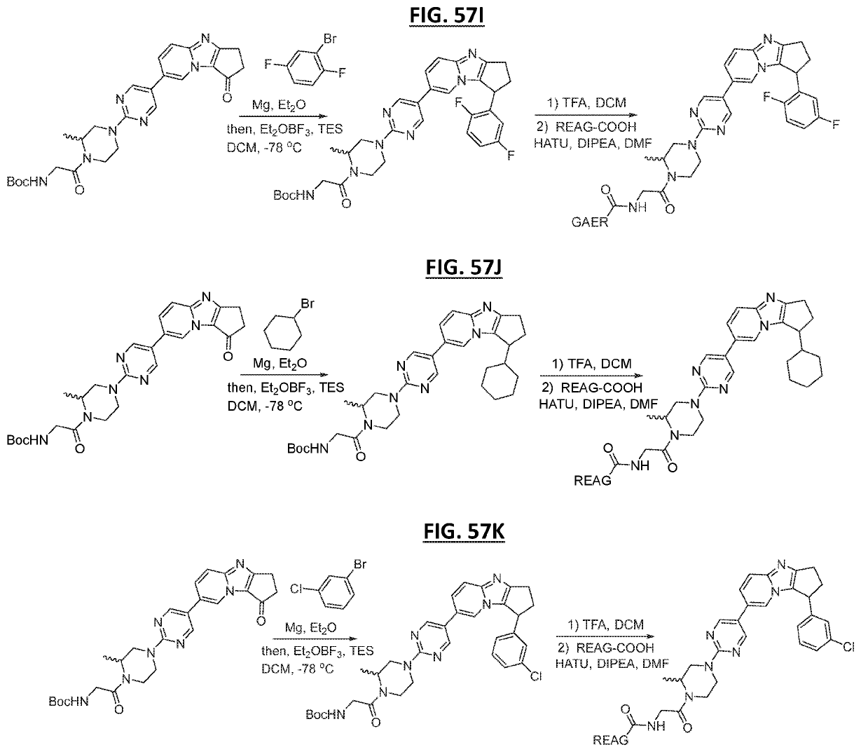

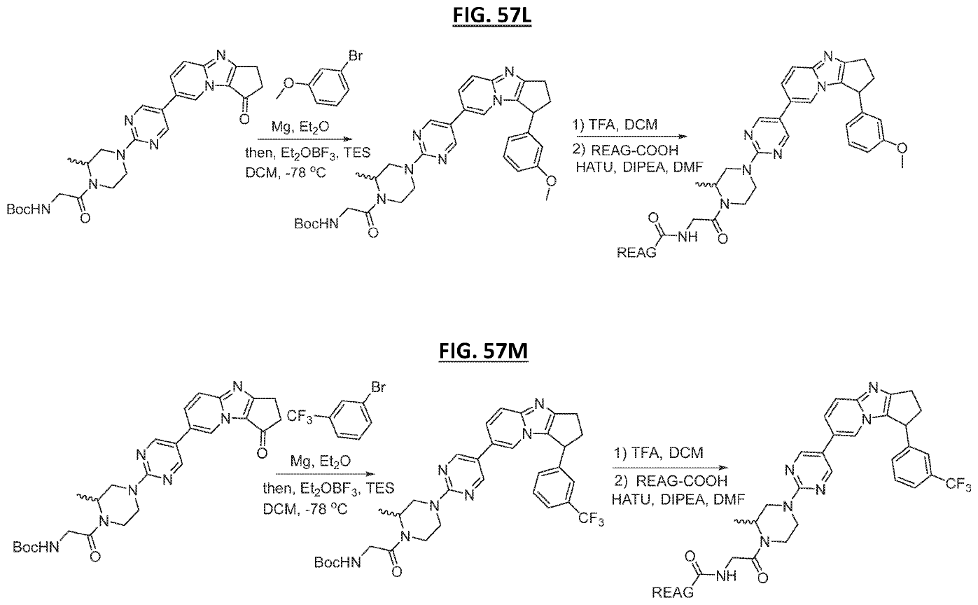

FIGS. 55A-55N and FIGS. 56A-560 illustrate the non-limiting synthesis of certain ASGPRBM groups and/or compounds of formula (I) or formula (Ia), using a MIF binder as a non-limiting Protein binder. Any protective group(s) in each intermediate and/or final product can be deprotected as appropriate

### Example 18: N1-(30-(((2R,3R,4R,5R,6R)-3-acetamido-4,5-dihydroxy-6-(hydroxymethyl)tetrahydro-2H-pyran-2-yl)oxy)-16-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-16-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-11,14,21-trioxo-3,6,9,18,25,28-hexaoxa-12,15,22-triazatriacontyl)-N4-(4-((E)-4-(2-(4-chloro-3-nitrophenyl)hydrazineylidene)-5-oxo-1-(4-phenylthiazol-2-yl)-4,5-dihydro-1H-pyrazol-3-yl)benzyl)succinamide (FIG. 15)

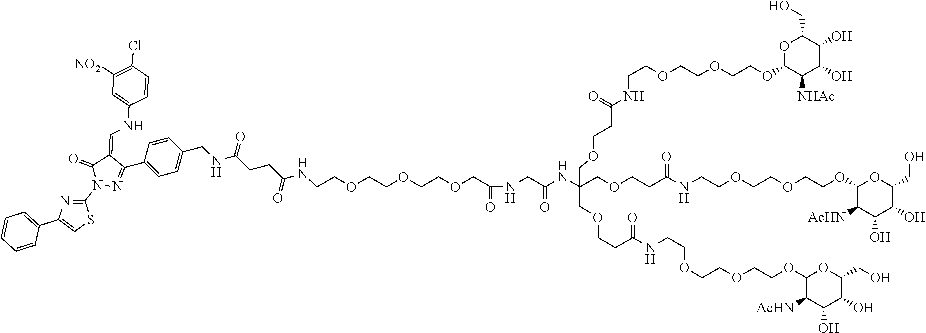 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)COCCOCCOCCNC(=O)CCC(=O)NCc2ccc(C3=NN(c4nc(-c5ccccc5)cs4)C(=O)/C3=C/Nc3ccc(Cl)c([N+](=O)[O-])c3)cc2)O[C@H](CO)[C@H](O)[C@@H]1O]

4-(((tert-butoxycarbonyl)amino)methyl)benzoic acid was treated with diimidazolyl ketone and magnesium ethyl malonate in THF to afford the intermediate ester, which was then treated with thiosemicarbazide in the presence of HCl, followed by stirring in ethyl acetate/ethanol, to give the intermediate bicyclic compound.

The amine was reprotected as the Fmoc using Fmoc chloride in the presence of organic base. The intermediate was then treated with sodium nitrite in the presence of strong acid, followed by treatment with 4-chloro-3-nitroaniline to give the diazo compound. Treatment of the intermediate with 2-bromo-1-phenylethan-1-one at elevated temperature in the presence of molecular sieves gave the cyclic product. Isomerization was then induced using UV light, followed by deprotection of the amine and reaction with succinic anhydride, to give the carboxylic acid product. This was then coupled with H₂N-(CH₂CH₂O)₃CH₂C(═O)NH-GN3 under standard amide coupling conditions, which upon treatment with sodium methoxide in methanol gave the bifunctional molecule.

### Example 19: 3,3′-((2-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-2-(2-(4-(5-((2-oxo-1,2-dihydrobenzo[cd]indole)-6-sulfonamido)-1H-indol-4-yl)benzamido)acetamido)propane-1,3-diyl)bis(oxy))bis(N-(2-(2-(2-(((2R,3R,4R,5R,6R)-3-acetamido-4,5-dihydroxy-6-(hydroxymethyl)tetrahydro-2H-pyran-2-yl)oxy)ethoxy)ethoxy)ethyl)propanamide) (FIG. 14)

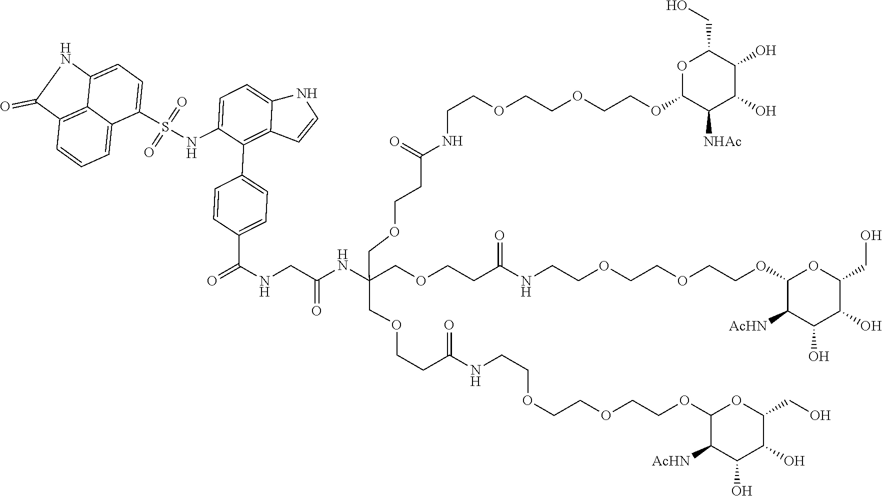 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)c2ccc(-c3c(NS(=O)(=O)c4ccc5c6c(cccc46)C(=O)N5)ccc4[nH]ccc34)cc2)O[C@H](CO)[C@H](O)[C@@H]1O]

Tert-butyl 5-amino-1H-indole-1-carboxylate was treated with NBS at low temperature in acetonitrile to give the brominated intermediate, which was then reacted with (4-(methoxycarbonyl)phenyl)boronic acid under cross coupling conditions (Pd catalyst, potassium carbonate, DMF, elevated temperature) to give compound A.

Separately, benzo[cd]indol-2(1H)-one was treated with chlorosulfuric acid to give the mono-substituted product, which then was reacted with A in the presence of pyridine in THF to give the intermediate methyl ester. This was deprotected by treatment with sodium hydroxide in MeOH/water, followed by treatment with TFA in DCM. The carboxylic acid was then reacted with NHS under standard coupling conditions to give the NHS ester, which was reacted with GN3 and treated with sodium methoxide to give the bifunctional molecule.

### Example 20: 3,3′-((2-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-2-(2-(2-(N-(2-(4-benzylpiperazin-1-yl)-2-oxoethyl)-N-(2,3-dimethylphenyl)sulfamoyl)benzamido)acetamido)propane-1,3-diyl)bis(oxy))bis(N-(2-(2-(2-(((2R,3R,4R.5R,6R)-3-acetamido-4,5-dihydroxy-6-(hydroxymethyl)tetrahydro-2H-pyran-2-yl)oxy)ethoxy)ethoxy)ethyl)propanamide) (FIG. 16)

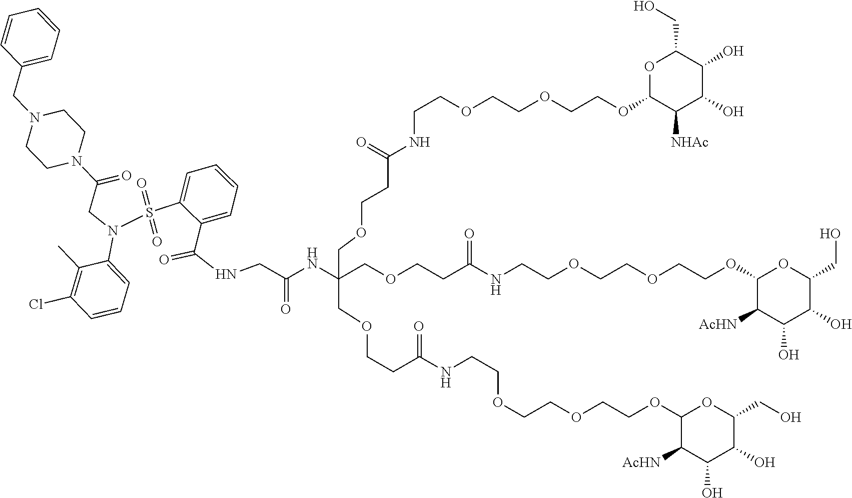 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)c2ccccc2S(=O)(=O)N(CC(=O)N2CCN(Cc3ccccc3)CC2)c2cccc(Cl)c2C)O[C@H](CO)[C@H](O)[C@@H]1O]

Piperazine was treated with benzyl bromide at elevated temperature in THF to give the monoalkylated intermediate A. Separately, methyl 2-(chlorosulfonyl)benzoate and 2,3-dimethylaniline were treated with pyridine in THF to afford the sulfonamide intermediate. This was then treated with sodium hydride in DMF, followed by tert-butyl 2-bromoacetate to give the alkylated diester product. The t-butyl ester was deprotected with TFA in DCM, then reacted under standard amide coupling conditions with intermediate A to give the monoester intermediate. This intermediate was then deprotected using sodium hydroxide in water/methanol, then reacted using EDCI, HOBt, and DIPEA in DMF with H₂N-GN3. Subsequent deprotection of the O-acyl groups with sodium methoxide in methanol gave the final compound.

### Example 21: N1-(30-(((2R,3R,4R,5R,6R)-3-acetamido-4,5-dihydroxy-6-(hydroxymethyl)tetrahydro-2H-pyran-2-yl)oxy)-16-(14-(((2R,3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-16-(14-(((3R,4R,5R,6R)-3-acetamido-4,5-dihydroxy-6-(hydroxymethyl)tetrahydro-2H-pyran-2-yl)oxy)-5-oxo-2,9,12-trioxa-6-azatetradecyl)-11,14,21-trioxo-3,6,9,18,25,28-hexaoxa-12,15,22-triazatriacontyl)-N4-(3-(((6,7-dimethyl-4-oxo-4H-chromen-3-yl)methyl)(2-(methyl((1-(3-(trifluoromethyl)phenyl)-1H-indol-3-yl)methyl)amino)ethyl)amino)propyl)succinamide (FIG. 17)

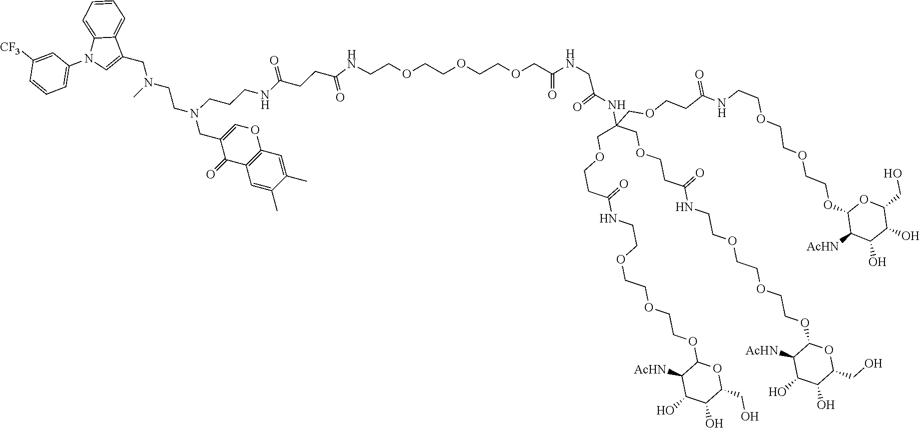 [SMILES: CC(=O)N[C@H]1[C@H](OCCOCCOCCNC(=O)CCOCC(COCCC(=O)NCCOCCOCCOC2O[C@H](CO)[C@H](O)[C@H](O)[C@H]2NC(C)=O)(COCCC(=O)NCCOCCOCCO[C@@H]2O[C@H](CO)[C@H](O)[C@H](O)[C@H]2NC(C)=O)NC(=O)CNC(=O)COCCOCCOCCNC(=O)CCC(=O)NCCCN(CCN(C)Cc2cn(-c3cccc(C(F)(F)F)c3)c3ccccc23)Cc2coc3cc(C)c(C)cc3c2=O)O[C@H](CO)[C@H](O)[C@@H]1O]

1H-indole was reacted with 1-bromo-3-(trifluoromethyl)benzene in the presence of copper (I) iodide and cesium carbonate in DMF at elevated temperature to give the condensed product. This was then reacted with POCl₃ in DMF and DCE at increasing temperature to give the aldehyde. N¹-(3-azidopropyl)-N²-methylethane-1,2-diamine was then reacted with this aldehyde and 6,7-dimethyl-4-oxo-4H-chromene-3-carbaldehyde to give the dimine, which was reduced using sodium triacetoxyborohydride. The azide was then reduced using triphenyiphosphine in water and THF, and reacted with succinic anhydride in the presence of organic base to give the resulting carboxylic acid. This was then treated with HBTU and DIPEA in DMF, followed by the addition of H₂N-(CH₂CH₂O)₃CH₂C(═O)NH-GN3. Deprotecting of the OAc groups with sodium methoxide gave the final compound.

### Example 22 (FIG. 12)

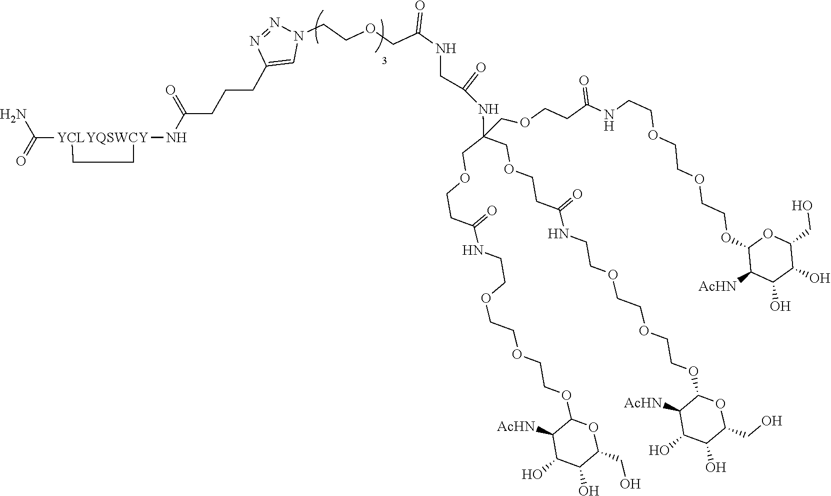

Peptide YCWSQYLCY (SEQ ID NO:132) was synthesized on rink amide resin following general solid phase synthesis protocols, then treated with 5-hexynoic acid in the presence of HATU and NMM in DMF to give the alkyne product, which was cleaved from resin using reagent L and cyclized overnight in pH8 buffer. This alkyne was reacted with N₃—(CH₂CH₂O)₃CH₂C(═O)NH-GN3 described previously under click chemistry conditions to afford the final compound.

### Example 23

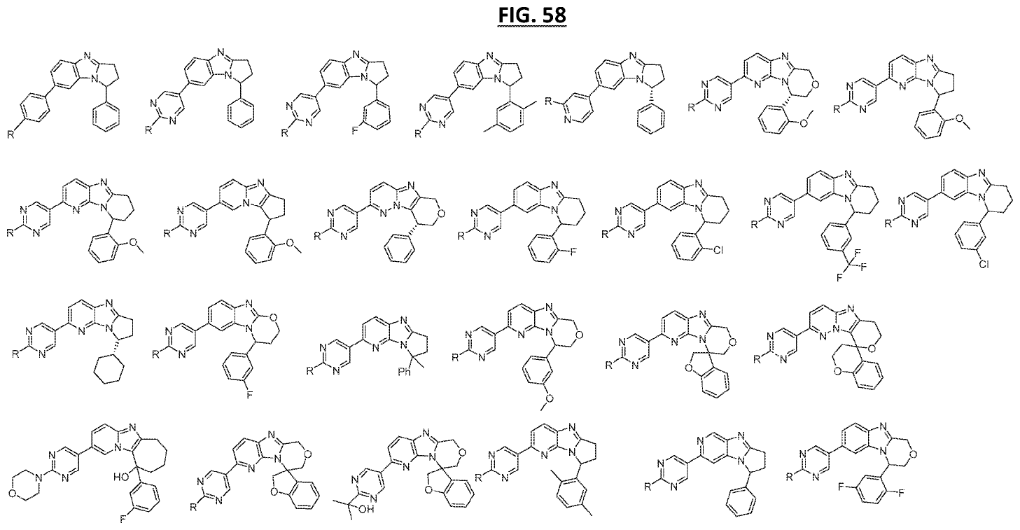

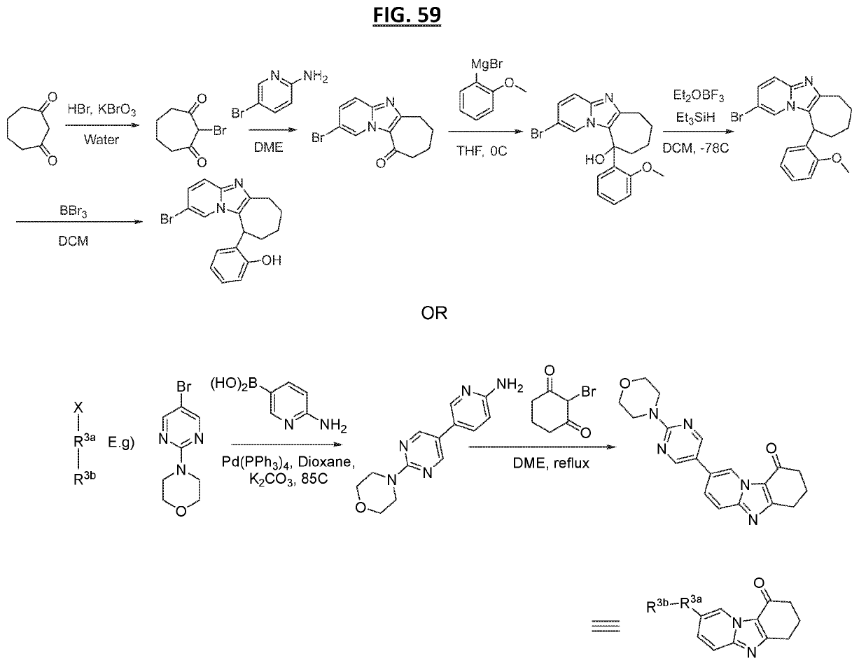

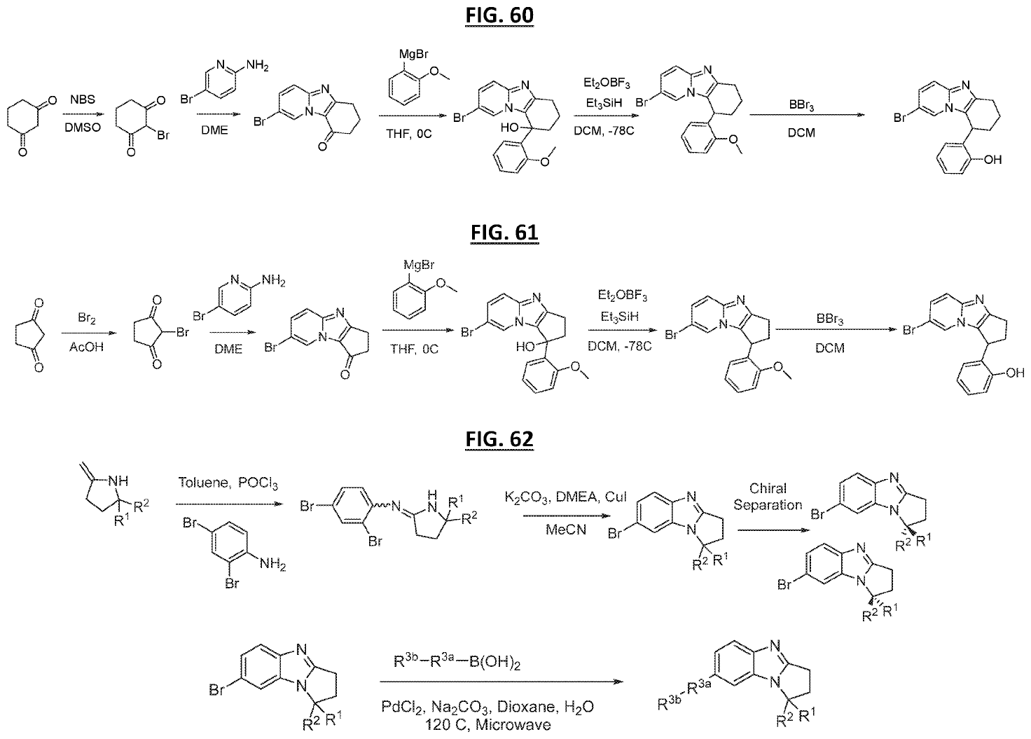

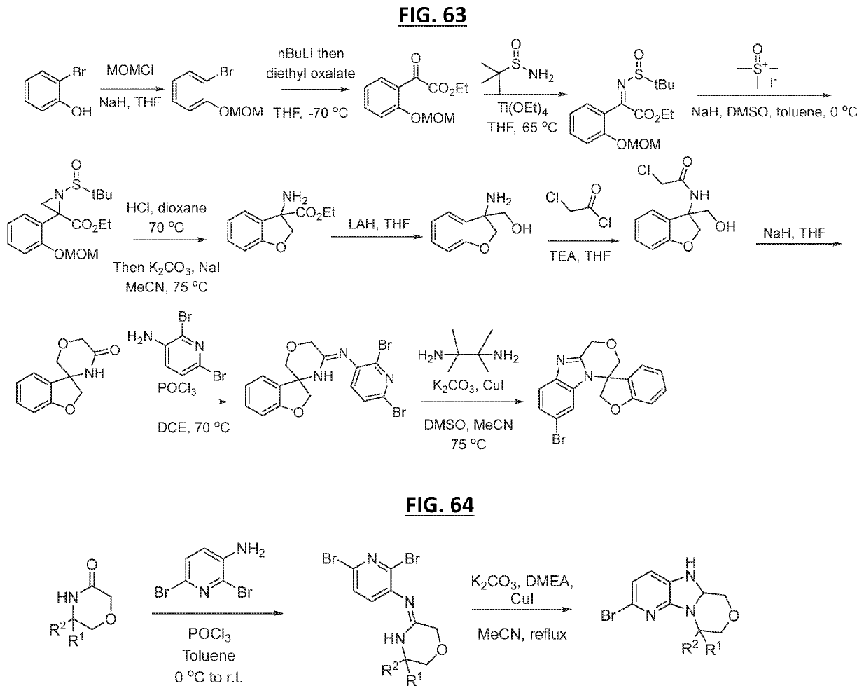

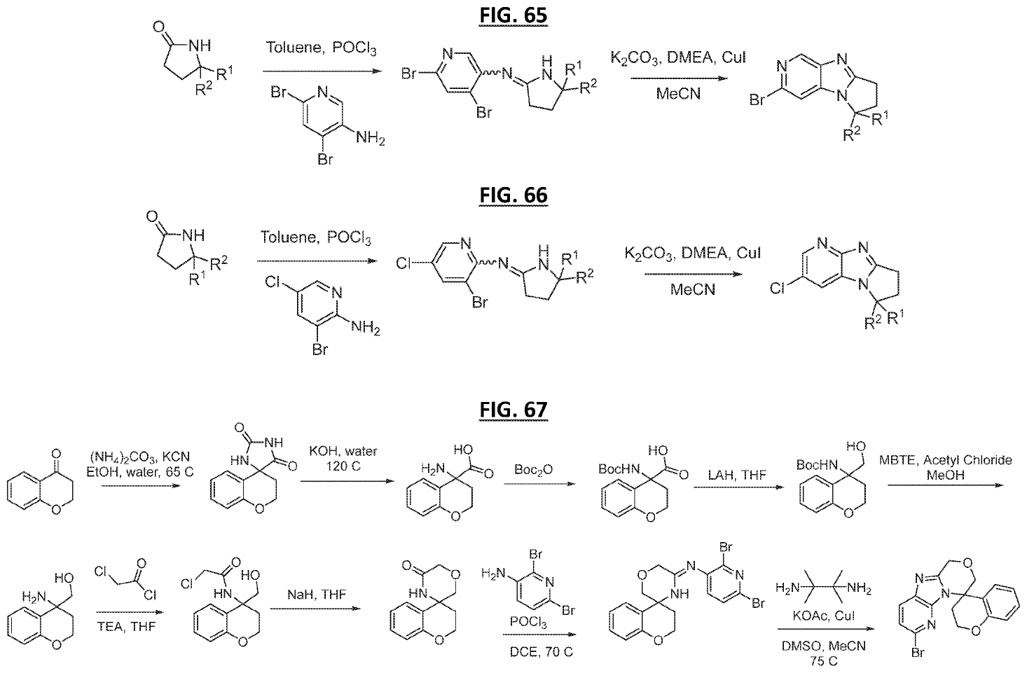

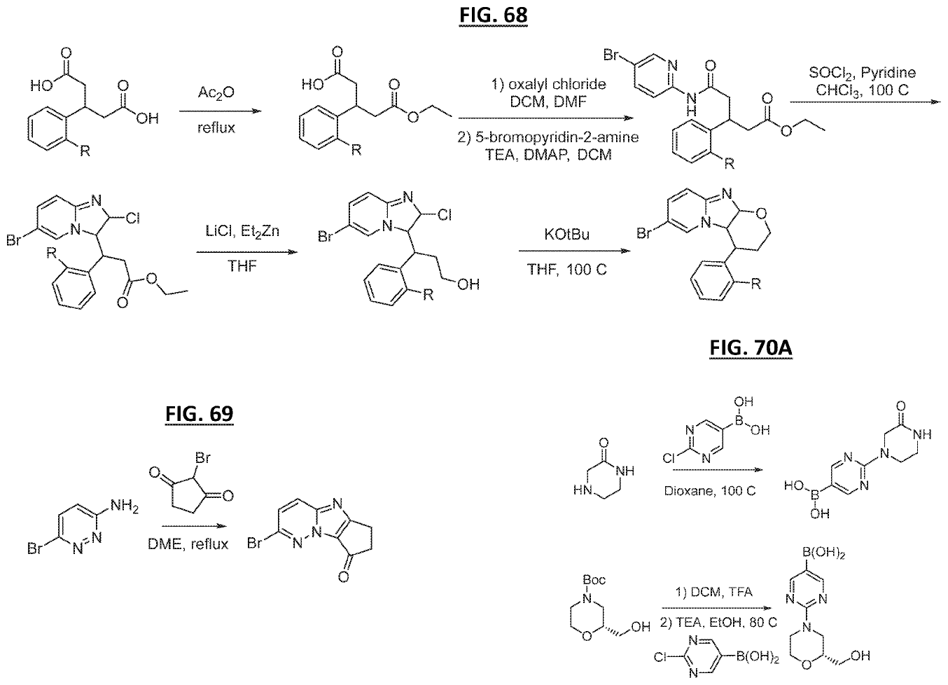

FIG. 58 illustrates certain compounds of formula (2b), wherein R represents R^{3b} in a non-limiting embodiment. FIGS. 59-69 illustrate a non-limiting synthesis of an intermediate that can be used to prepare a compound of formula (2b).

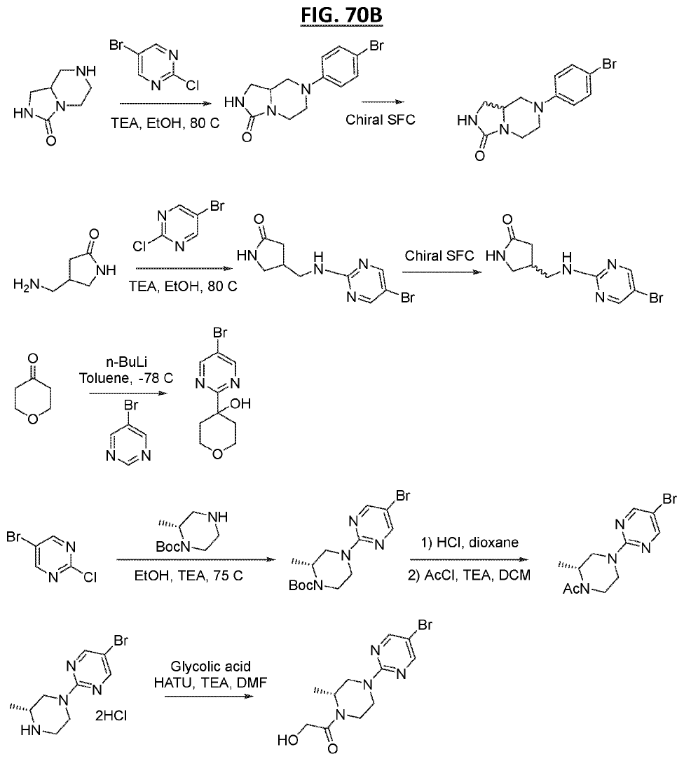

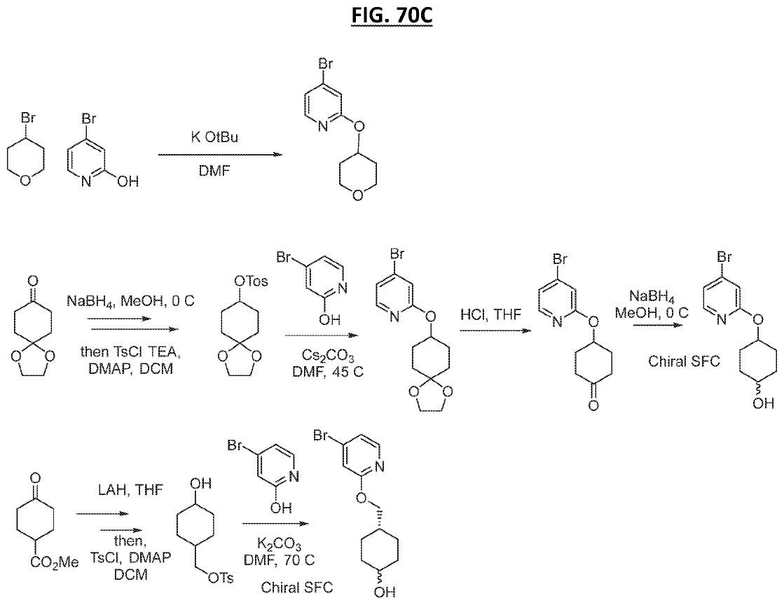

FIGS. 70A-70C illustrate non-limiting syntheses of certain intermediates that can be used to prepare a compound of formula (2b) (providing R³) or of formula (2c) (providing R²).

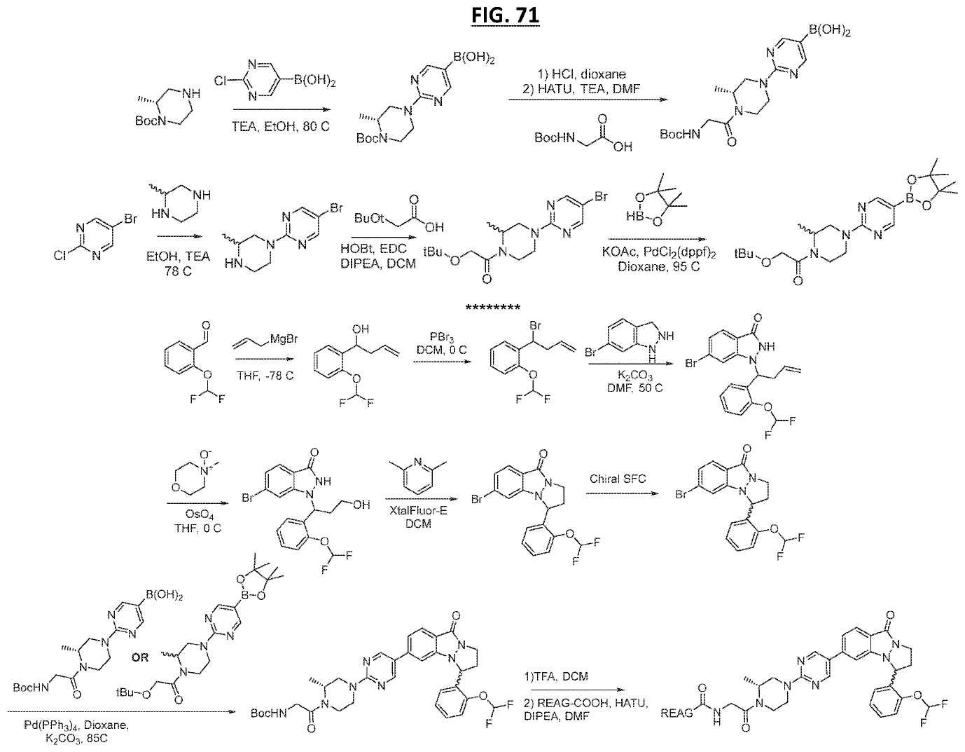

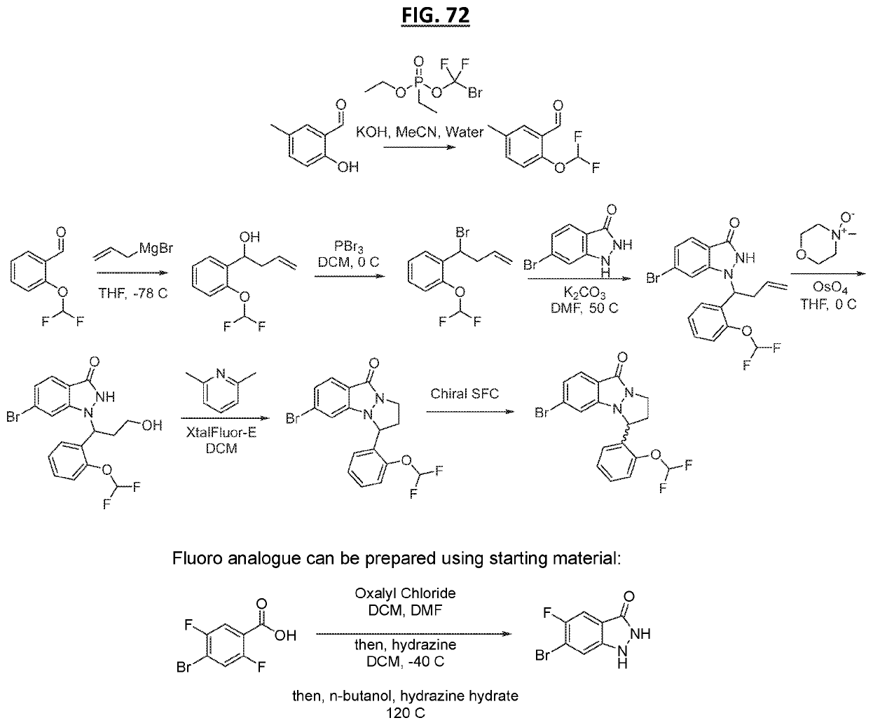

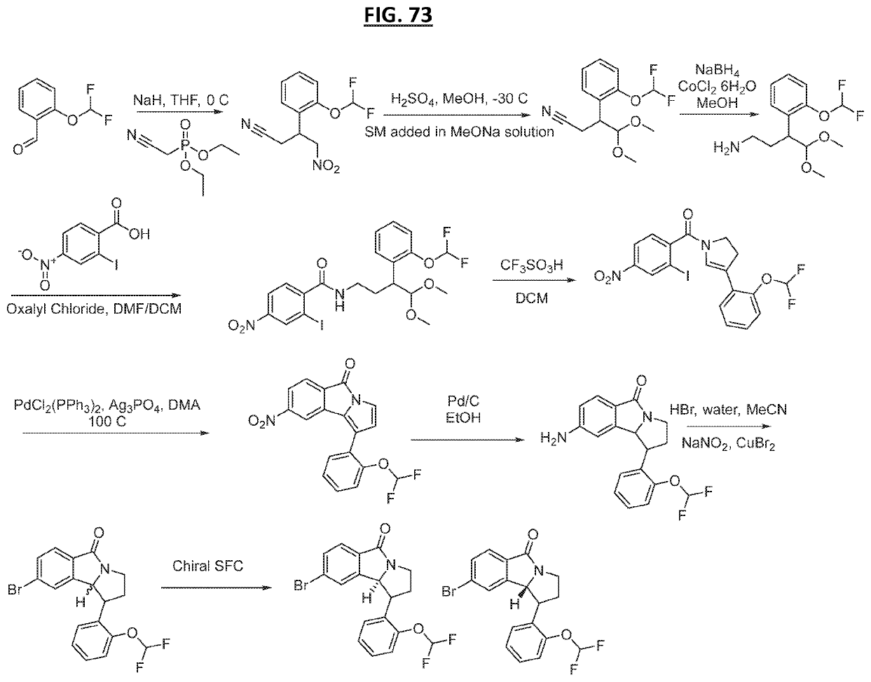

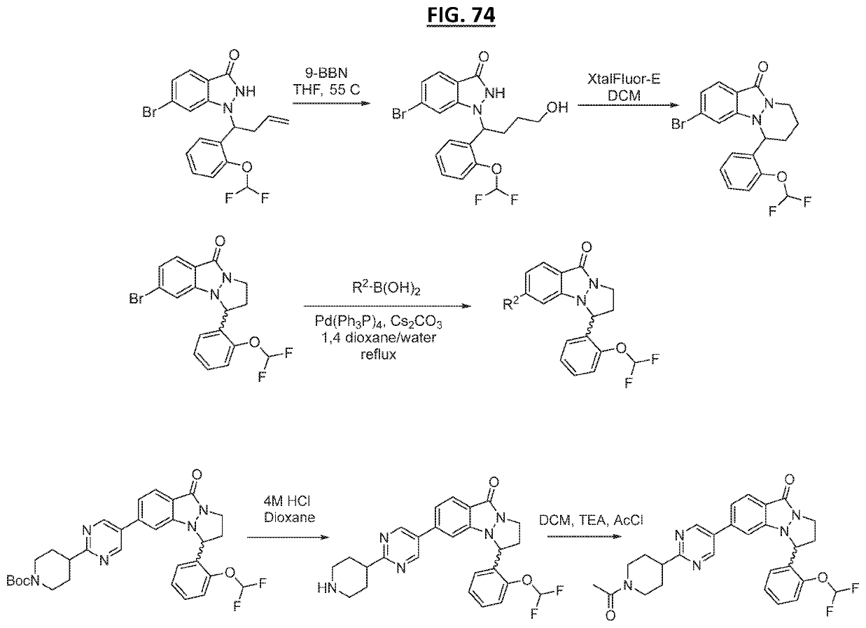

FIG. 71 illustrates a non-limiting synthesis of certain compounds of the disclosure, such as but not limited to formula (2c). FIGS. 72 and 73 illustrate a non-limiting synthesis of an intermediate that can be used to prepare a compound of formula (2c). FIG. 74 illustrates a non-limiting synthesis of a compound of formula (2c).

### Example 24: Bi-Functional Compounds as Degraders of Autoantibodies

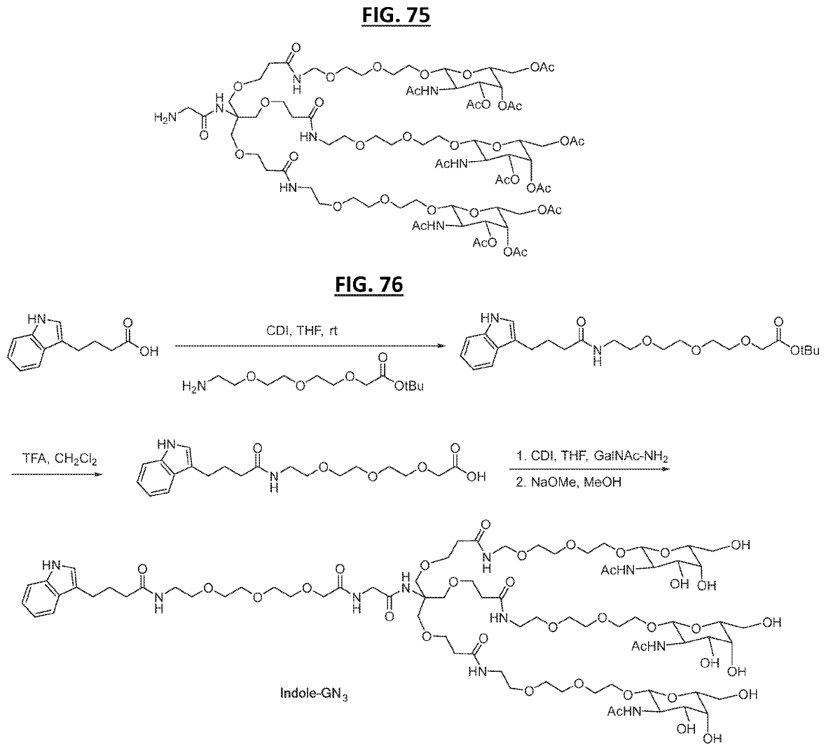

FIG. 75 illustrates the structure of GalNAc-NH₂.

FIG. 76 illustrates a non-limiting synthesis of Indole-GN3, a bifunctional molecule that targets the degradation of human IgG/IgE/IgM.

FIG. 77 illustrates a non-limiting synthesis of AMD-GN3, a bifunctional molecule that targets the selective degradation of human IgG.

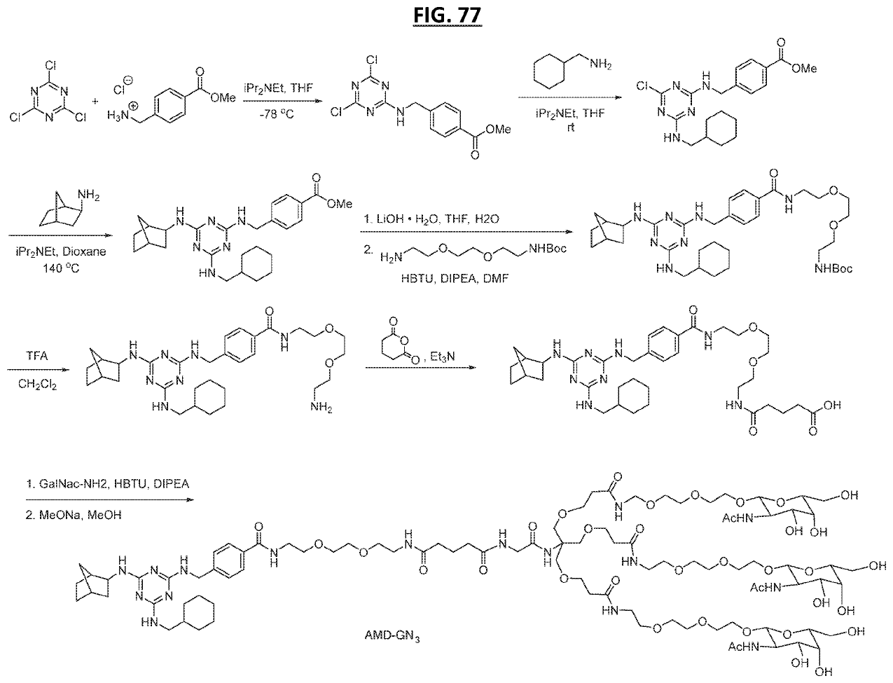

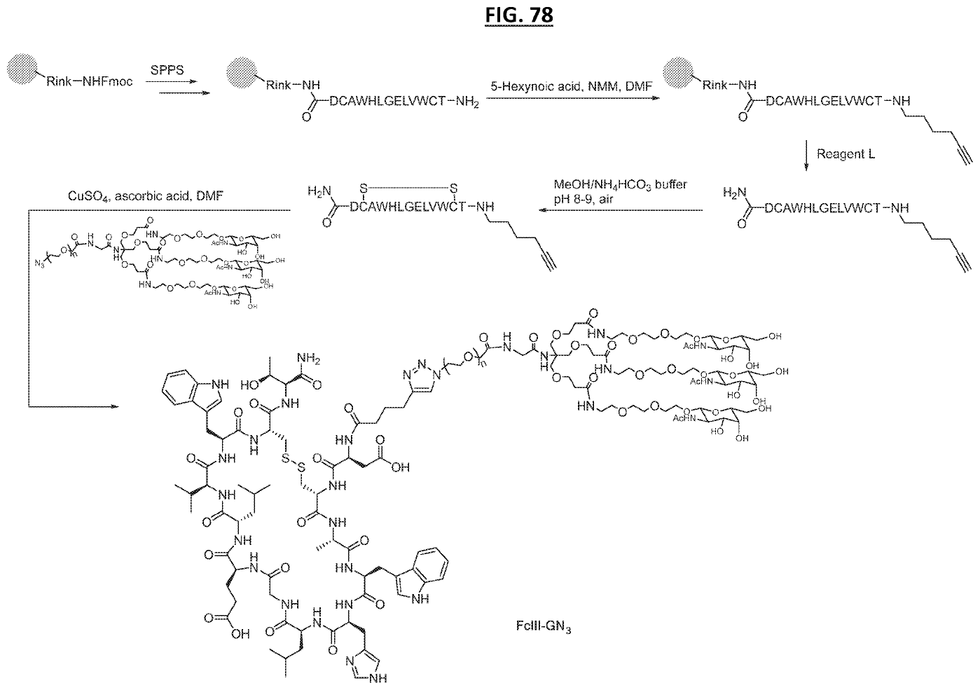

FIG. 78 illustrates a non-limiting synthesis of FcIII-GN3, a bifunctional molecule that targets the selective degradation of human IgG.

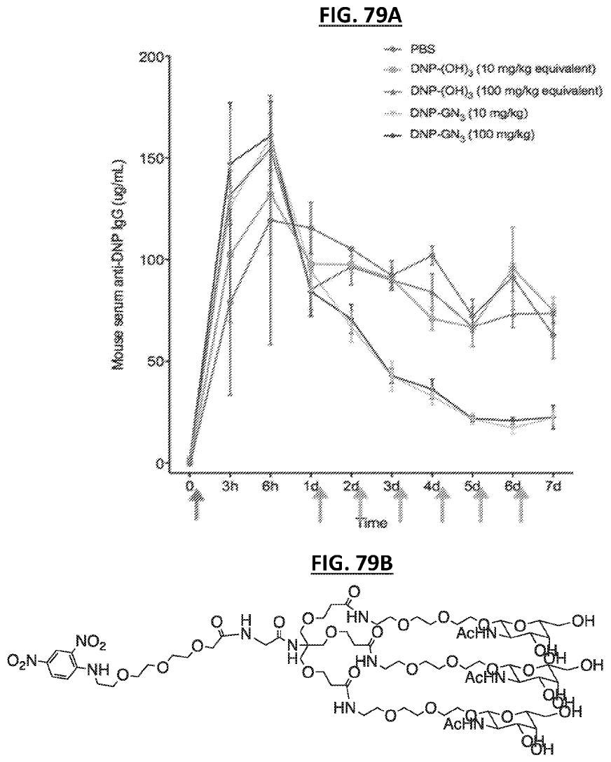

FIGS. 79A-79B illustrate in vivo data that demonstrate cleavage of anti-DNP IgG in mouse serum mediated by DNP-GN3. FIG. 79A: Mouse experiment showing that bifunctional molecule DNP-GN3 can induce degradation of injected anti-DNP IgG antibodies in mouse serum while the negative control molecule or vehicle control did not show such effect. Purple arrow: Mice were injected with anti-DNP IgG antibodies i.p., Green arrows: Mice were injected i.p. with PBS (vehicle), DNP-(OH)₃ (negative control) or DNP-GN3. FIG. 79B: Structure of DNP-GN3.

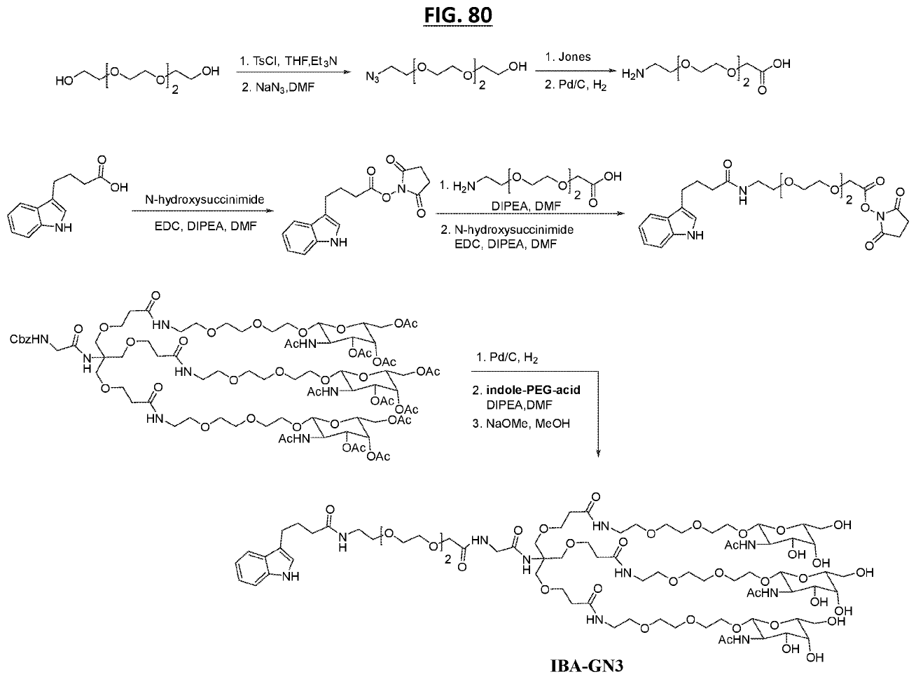

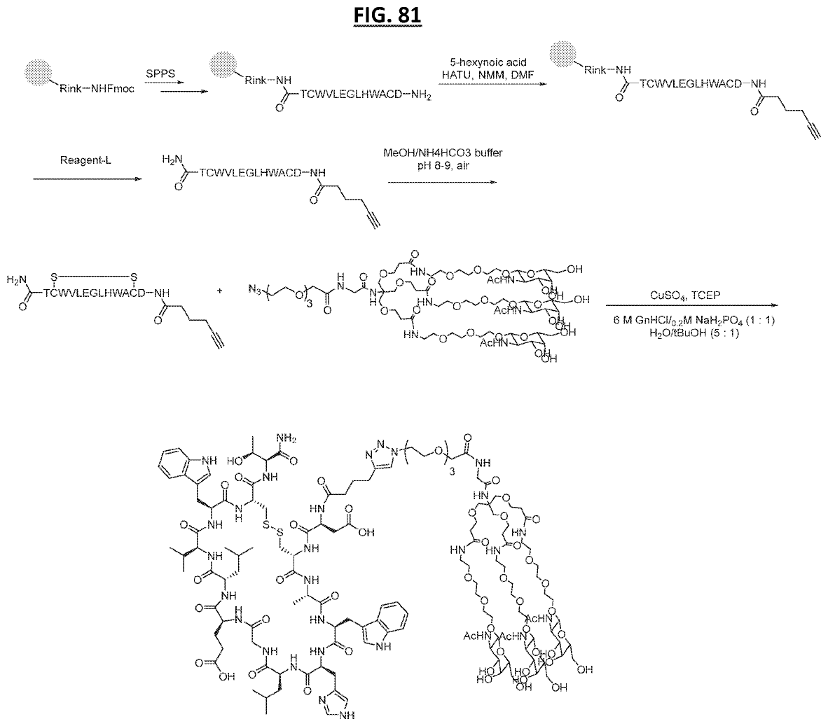

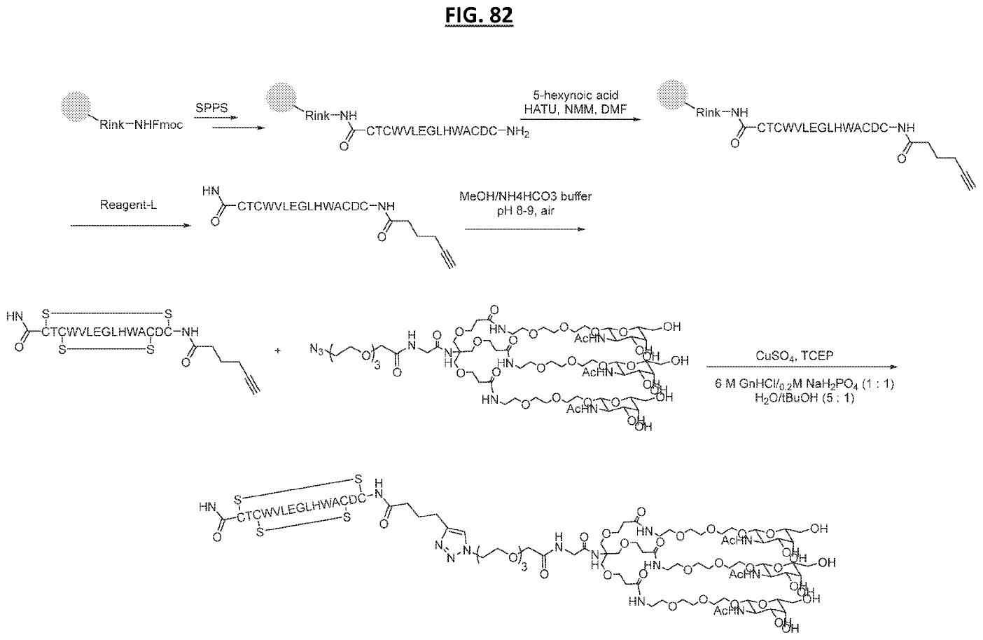

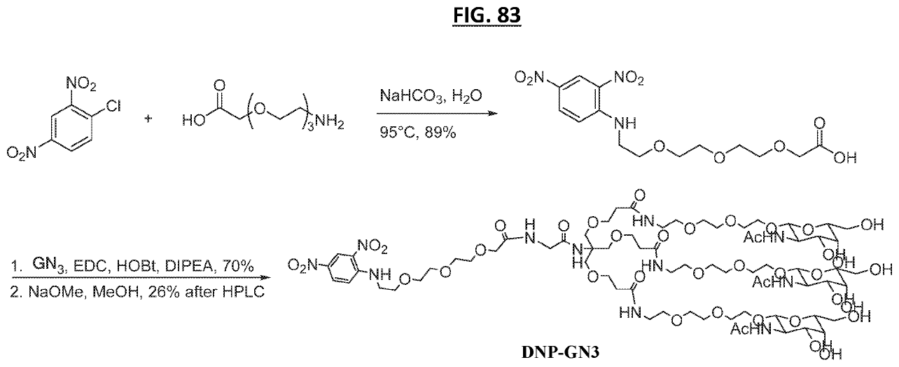

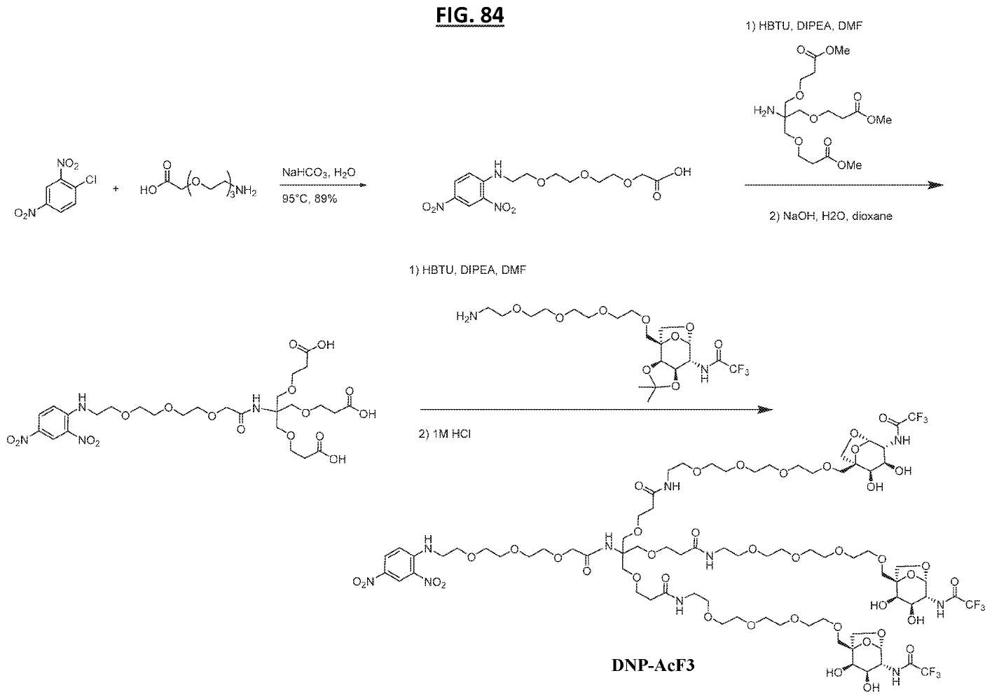

FIGS. 80-84 illustrate non-limiting synthesis of certain bifunctional compounds of the disclosure. FIG. 81 depicts the synthesis of a bifunctional compound comprising a DCAWHLGELVWCT (SEQ ID NO:139) peptide (optionally C-C cyclized). FIG. 82 depicts the synthesis of a bifunctional compound comprising a CDCAWHLGELVWCTC (SEQ ID NO:140) peptide (optionally C-C cyclized).

### Example 25: Synthesis of Negative Control α-DNP Antibody Binding Compound DNP-OH3 (FIG. 85)

The synthesis of DNP-OH3 begins with an HBTU-mediated amide bond formation between the tri-carboxylic acid and three equivalents of the commercially available hydroxy amine 2-[2-(2-aminoethoxy)ethoxy]ethanol, affording the Cbz-protected intermediate. The intermediate is reduced to afford an amine which undergoes an HBTU-mediated cross coupling with a carboxylic acid, forming the final compound DNP-OH3.

### Example 26: DNP-GN3 Mediates the Endocytosis of α-DNP Antibody

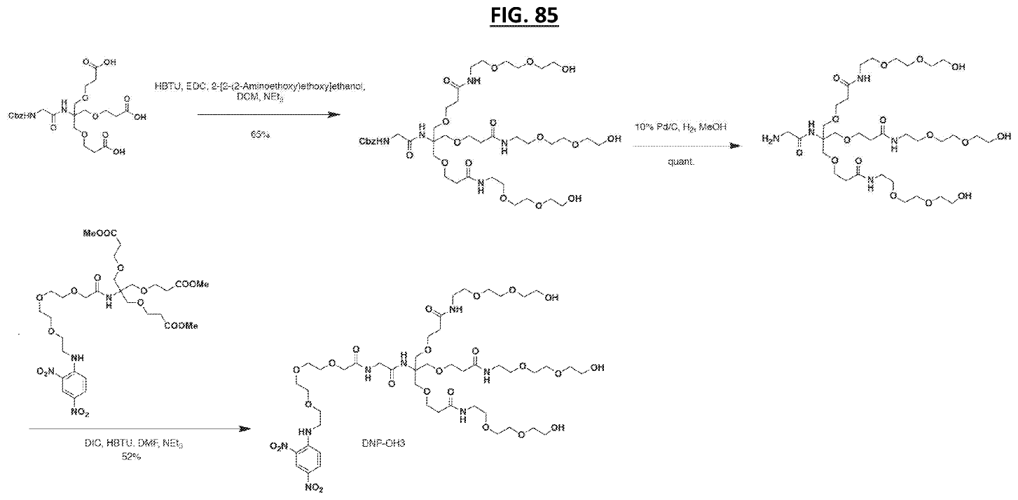

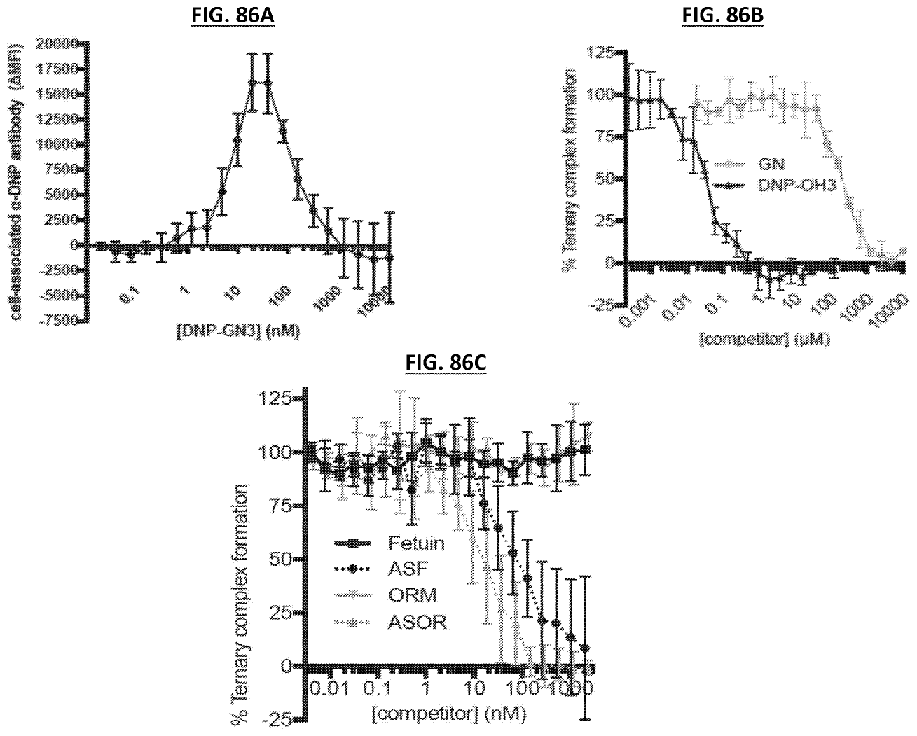

It was investigated whether DNP-GN3 could mediate the formation of a ternary complex between a fluorescently labeled α-DNP antibody and ASGPR on the surface of immortalized human hepatocyte HepG2 cells in suspension. The extent of fluorescently-labeled antibody association with cells was found to be dependent on the concentration of DNP-GN3, with concentrations of 7.4 nM and 0.12 μM eliciting half-maximal fluorescence association (FIG. 86A). The observed bell-shaped response to DNP-GN3 concentration is consistent with the prozone effect commonly observed in systems wherein a ternary complex is formed.

Cell-associated fluorescence was found to be inhibited by reagents that bind competitively to either ASGPR or α-DNP antibody. Cellular fluorescence was decreased by increasing concentrations of both DNP-OH3 (IC₅₀=36 nM) and monomeric GalNAc (GN) sugar (IC₅₀=0.20 mM) (FIG. 86B). Proteins that bind specifically to ASGPR also decreased DNP-GN3-mediated cellular fluorescence (FIG. 86C). The serum proteins fetuin and orosomucoid (ORM) bind to ASGPR only after they have been desialylated to produce asialofetuin (ASF, reported Kᵢ=17 nM) and asialoorosomucoid (ASOR, reported Kᵢ=1.7 nM), respectively. Both ASF (IC₅₀=65 nM) and ASOR (IC₅₀=17 nM) decreased cellular fluorescence, while fetuin and ORM did not. Taken together, these data suggest that DNP-GN3 mediates the formation of a ternary complex between α-DNP antibody and ASGPR on the hepatocyte surface.

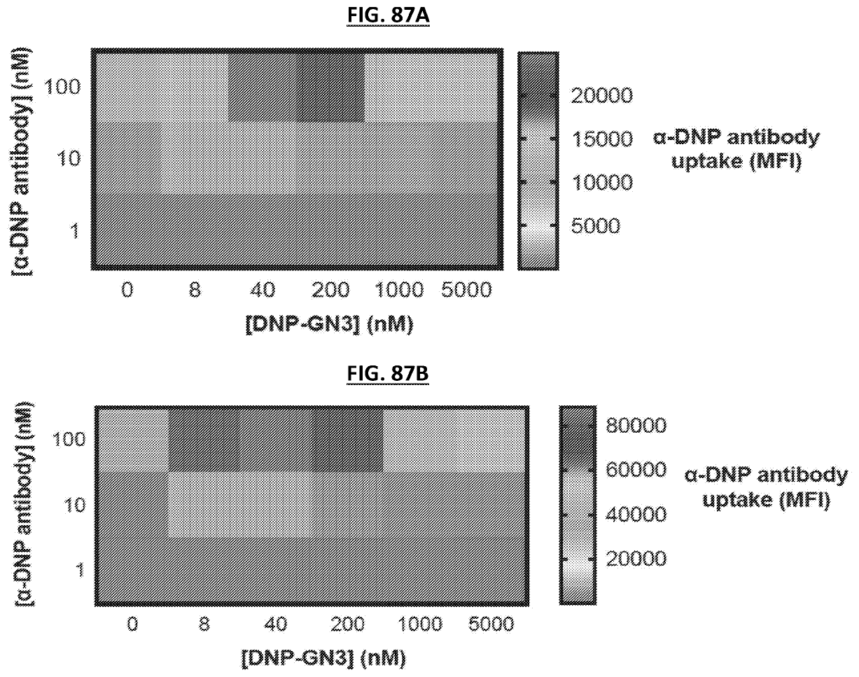

It was next explored whether DNP-GN3 mediates the endocytosis of fluorescently labeled α-DNP antibody. It was observed that the intracellular fluorescence of adherent HepG2 cells was dependent on the concentration of both DNP-GN3 and α-DNP antibody (FIG. 87A), and that intracellular fluorescence increased over time (FIG. 87B).

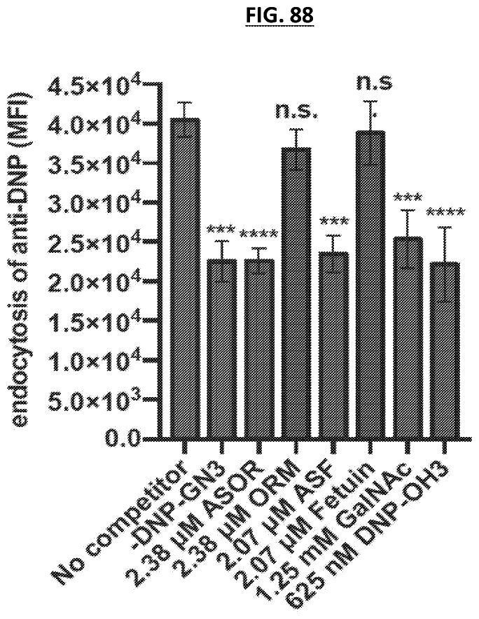

The observed increase in intracellular fluorescence mediated by DNP-GN3 was inhibitable by reagents that were previously shown to interfere with ternary complex formation (FIG. 88). At high concentrations, ASOR, ASF, GalNAc, and DNP-OH3 significantly decreased DNP-GN3-mediated α-DNP antibody endocytosis. In contrast, no decrease in α-DNP antibody endocytosis was observed when cells were treated with the proteins ORM or fetuin, which do not bind strongly to ASGPR. These data are consistent with a model in which DNP-GN3 mediates antibody uptake by engaging ASGPR.

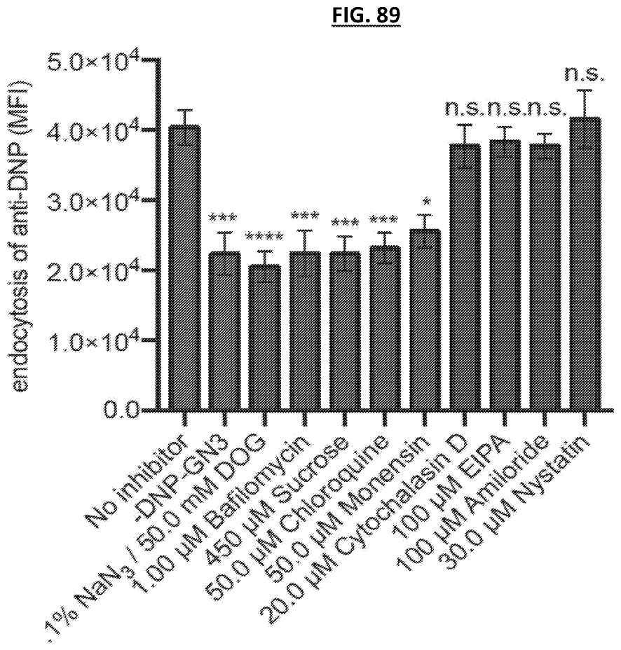

To explore the cellular mechanism of antibody uptake, cells were treated with chemical inhibitors of several endocytic pathways (FIG. 89). Combined treatment with the global endocytosis inhibitors sodium azide and 2-deoxyglucose (DOG) significantly decreased intracellular fluorescence. Fluorescence was also significantly decreased by reagents that inhibit clathrin-dependent endocytosis by disrupting endosomal formation(sucrose) and/or acidification (bafilomycin, chloroquine, and monensin). In contrast, inhibitors of caveolae-mediated endocytosis (nystatin) and macropinocytosis and phagocytosis (cytochalasin D, 5-(N-Ethyl-N-isopropyl)amiloride (EIPA), and amiloride) did not significantly decrease cellular fluorescence. The pattern of inhibition observed in these experiments is consistent with an endocytic mechanism that relies on clathrin, but not other endocytic pathways. Given that previous reports have shown that endocytosis mediated by ASGPR is dependent on clathrin (Oka, J. et al., Journal of Biological Chemistry, 1989, 264:12016-12024; Schwartz, A. L. et al., The Journal of cell biology, 1984, 98:732-738) this data further supports the participation of ASGPR in antibody endocytosis mediated by DNP-GN3.

### Example 27: DNP-GN3 Mediates the Degradation of α-DNP Antibody

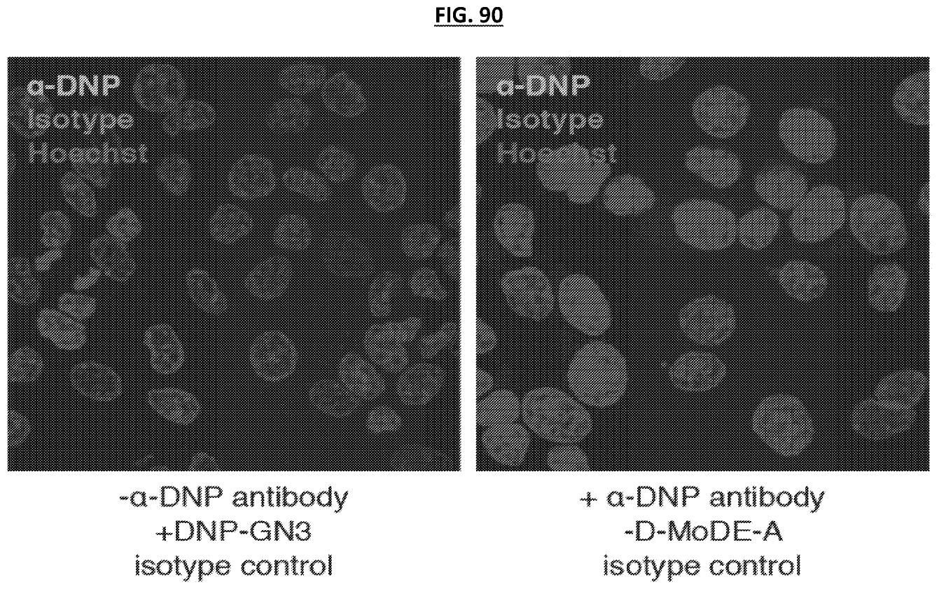

Having demonstrated that DNP-GN3 mediates the endocytosis of α-DNP antibodies, the subcellular localization of endocytosed fluorescently labeled α-DNP antibody was investigated. Accumulation of intracellular antibody-derived fluorescence was found to depend on the presence of both DNP-GN3 and α-DNP antibody (FIG. 90).

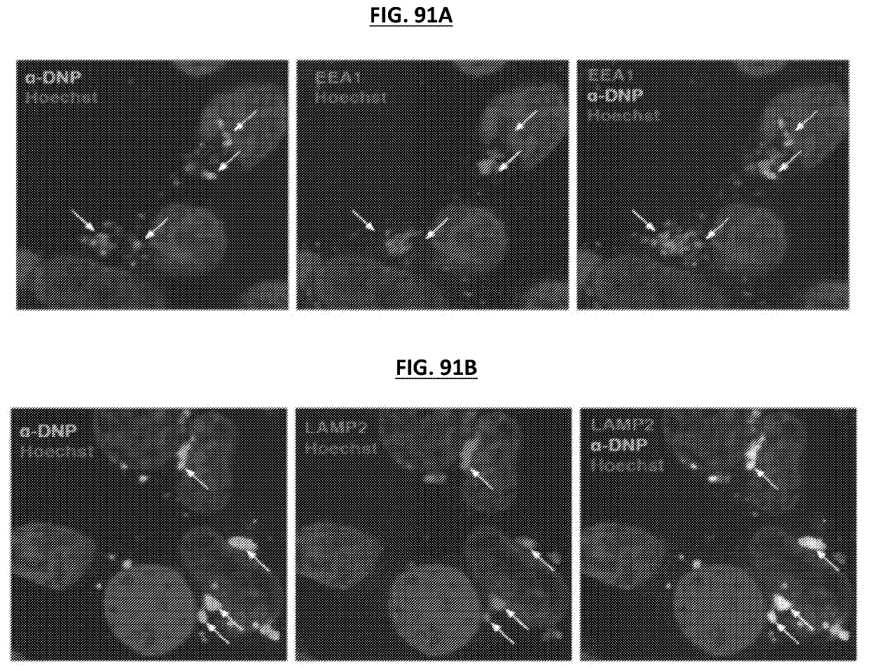

No colocalization of endocytosed α-DNP antibody with the early endosome marker EEA1 was observed in cells after 12 hours (FIG. 91A). In contrast, strong colocalization of α-DNP antibody was observed with the lysosome membrane protein LAMP2 (FIG. 91B). Taken together, these microscopy data suggest that trafficking to lysosomes is rapid on the time scale of this experiment (12 hours) and that the majority of endocytosed antibody was present in mature endocytic compartments.

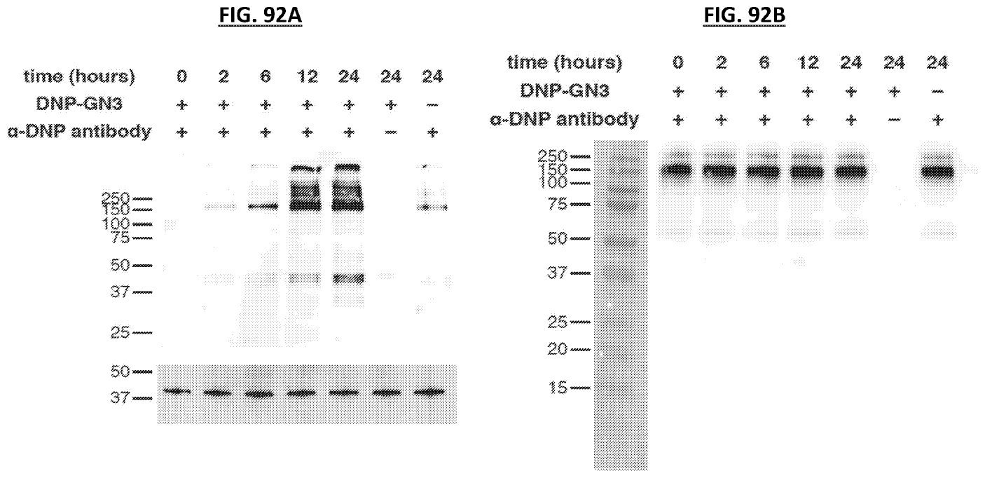

In addition, western blotting with an antibody directed to Alexa Fluor 488 was used to determine if endocytosed fluorescently labeled α-DNP antibodies are degraded in vitro. Lysates from HepG2 cells treated with both DNP-GN3 and α-DNP antibody were found to accumulate fluorophore in a time- and DNP-GN3-dependent manner (FIG. 92A). Signal arising from Alexa Fluor 488 in cell lysates was associated with only full-length antibody after two hours of incubation. After six hours, a lower molecular weight fluorophore-associated protein fragment between 37 and 50 kDa began to appear. An additional protein fragment with a molecular weight between 25 and 37 kDa was observed after 24 hours. It is believed that these fluorophore-associated protein fragments are degradation products resulting from lysosomal proteolysis of α-DNP antibodies, collectively indicating that endocytosed α-DNP antibody is degraded in HepG2 cells. Furthermore, no degradation products were observed in the cell culture supernatant (FIG. 92B) suggesting that antibody degradation is taking place in or on HepG2 cells. Together with the above immunofluorescence studies showing the colocalization of α-DNP antibody-derived fluorescence with lysosomes, these studies support that α-DNP antibody degradation is mediated by lysosomal proteases. Lysosomal degradation is further consistent with a mechanism dependent on ASGPR.

### Example 28: DNP-GN3 Mediates the Depletion of α-DNP Antibody In Vivo

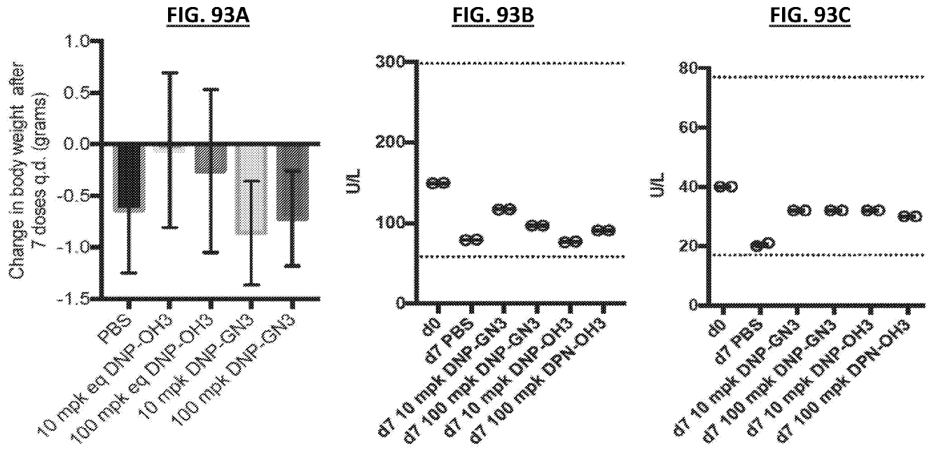

Having demonstrated that DNP-GN3 mediates the degradation of α-DNP antibodies in vitro, the viability of the MoDE-A (Molecular Degraders of Extracellular proteins through the Asialoglycoprotein receptor (ASGPR)) technology in vivo was evaluated. A dose of 1 mpk DNP-GN3 was found to be bioavailable via IP dosing in nude mice, with the maximal serum concentration reached after 1 h and a measured half-life in serum of 0.67 h. DNP-GN3 was well-tolerated up to doses of 100 mpk, with no significant differences in body weight or serum liver enzyme levels between control and treatment groups (FIGS. 93A-93C). These results supported the suitability of DNP-GN3 for more detailed studies in vivo.

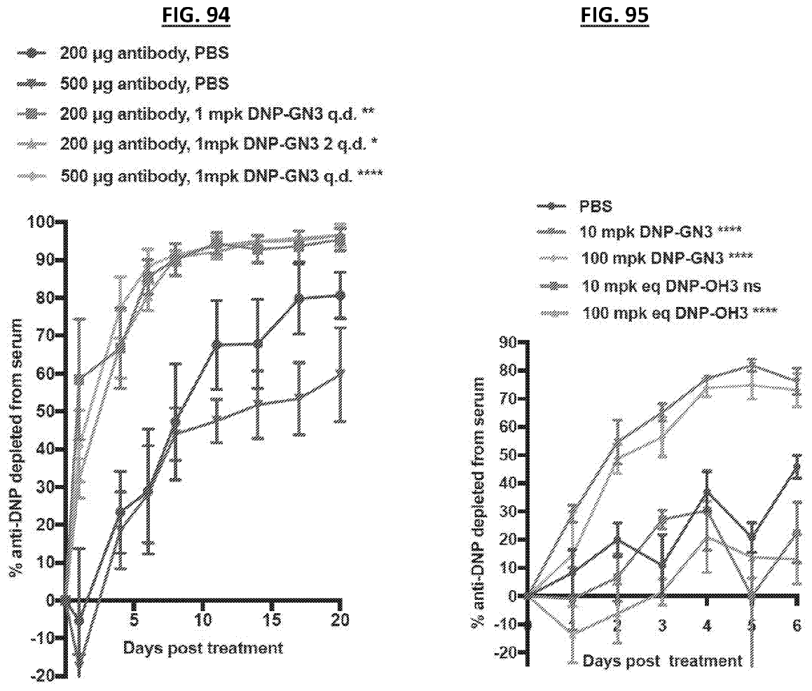

Treatment with DNP-GN3 was found to accelerate the depletion of monoclonal mouse IgG2 α-DNP antibodies from serum in nude mice in vivo (FIG. 94). Following an initial dose of 200 μg α-DNP antibody, both daily and twice-daily injections of 1 mpk DNP-GN3 significantly reduced antibody levels compared to PBS treatment over 21 days. Daily treatment with DNP-GN3 also gave a significant decrease in antibody levels following an initial antibody dose of 500 μg, indicating that DNP-GN3 is effective over a range of target protein concentrations in vivo.

No accelerated antibody depletion from serum was observed following treatment with the negative control compound DNP-OH3, which binds to the α-DNP antibody but not ASGPR (FIG. 95). Unexpectedly, a dose of 100 mpk DNP-OH3 resulted in a small but statistically significantly decrease in antibody clearance compared to the PBS control. It is hypothesized that this is a result of an increase in the antibody's hydrodynamic radius due to the DNP-OH3 PEG chains, which may increase its half-life in vivo. Related phenomena have been widely observed for pegylated proteins, and have been exploited for increasing the half-lives of various therapeutic modalities.

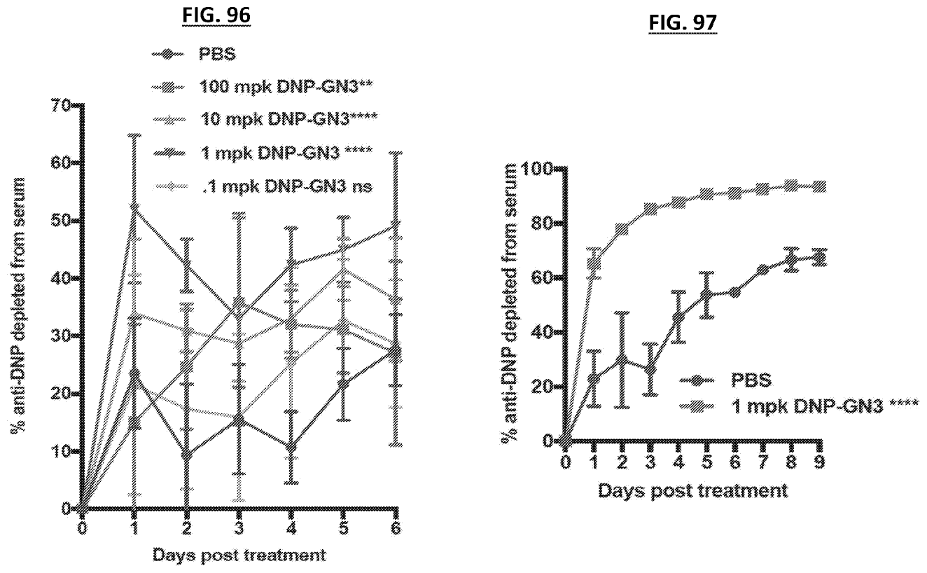

Single doses of DNP-GN3 were also found to be efficacious at mediating α-DNP antibody depletion, albeit less effectively than daily dosing (FIG. 96). Treatment with either 1 mpk or 10 mpk DNP-GN3 were found to be the most effective, with 52% and 34% of α-DNP antibody depleted from serum respectively 24 hours after a single dose, versus 24% depletion in the vehicle control. Significant depletion was also observed following a dose of 100 mpk of DNP-GN3. Therefore, it was concluded that DNP-GN3 is able to mediate the depletion of a monoclonal antibody from serum, and functions across a wide range of target protein concentrations and dosing regimens.

DNP-GN3 was also found to be efficacious in depleting polyclonal α-DNP antibody from serum collected from mice immunized with DNP-keyhole limpet haemocyanin (KLH) (FIG. 97). Following daily treatment with DNP-GN3, significantly more polyclonal α-DNP antibody was removed from serum compared to the PBS control at each time point. Thus, the small molecule DNP-GN3 is not restricted in function to only monoclonal mouse antibodies, but is also effective at removing polyclonal α-DNP antibodies from circulation in mice.

### Example 29: DNP-AF3 Mediates the Endocytosis of α-DNP Antibody

After demonstrating that DNP-GN3, which utilizes a trivalent GalNAc motif to bind to ASGPR, is effective at mediating target protein endocytosis and degradation both in vitro and in vivo, experiments were conducted with DNP-AF3. DNP-AF3 utilizes trivalent display of a higher affinity ASGPR ligand to engage the receptor.

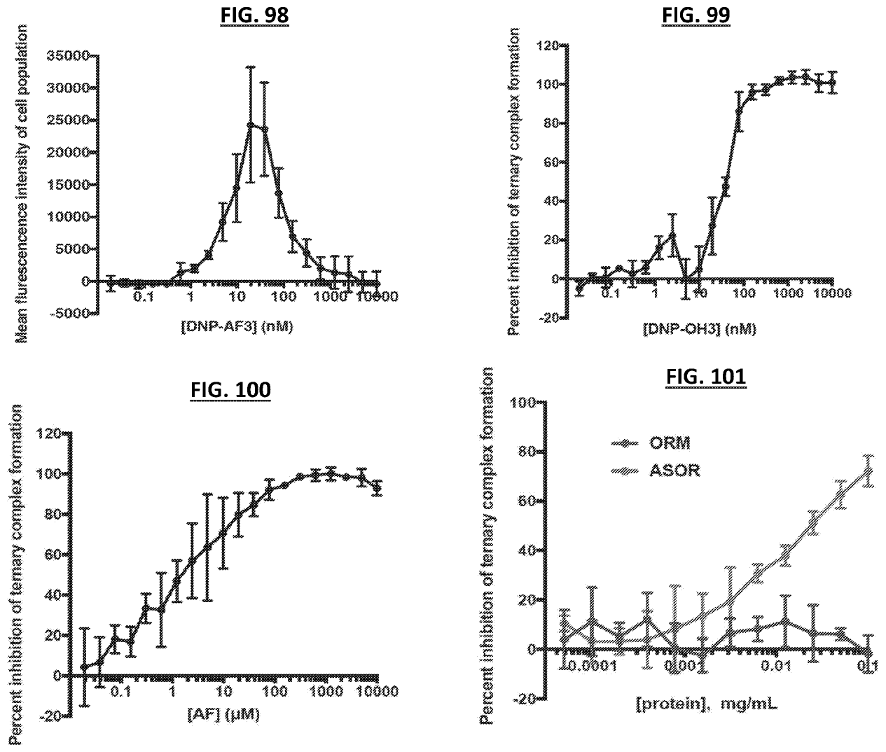

First, the effect of DNP-AF3 concentration on α-DNP antibody association with cells was determined (FIG. 98). In these experiments, a constant amount of α-DNP antibody and cells was incubated with various concentrations of DNP-AF3. The mean fluorescence intensity of the cell population was measured using flow cytometry. In order to account for background cellular fluorescence and non-DNP-AF3 mediated antibody association with cells, the mean fluorescence intensity of a sample of cells which had been treated with α-DNP antibody but not DNP-AF3 was subtracted from each data point.

Maximal ternary complex formation was observed at DNP-AF3 concentrations of 19.5 and 39.1 nM. These observations with DNP-AF3 showed similar trends to treatment with DNP-GN3, which exhibited maximal ternary complex formation at concentrations of 20 and 40 nM. A decrease in cellular fluorescence was also observed at high concentrations of DNP-AF3, which is consistent with ternary complex formation. Based on these data, it was concluded that DNP-AF3 mediates the association of α-DNP antibody with HepG2 cells.

Next, whether increasing concentrations of a competitive binder of the α-DNP antibody inhibits ternary complex formation was investigated. At a concentration of 40 nM DNP-AF3, a stable ternary complex formation with minimal variability was observed between experimental replicates within the same experimental group. However, different preparations of the HepG2 cells and/or compound gave varying mean fluorescence intensity for the cell population between different independent experimental replicates. Therefore, for competition studies, inhibition of ternary complex formation was reported rather than mean fluorescence intensity of the cell population. 100% ternary complex formation is corrected to the fluorescence of a cell population treated with both 40 nM DNP-AF3 and 100 nM α-DNP antibody, while 0% ternary complex formation is corrected to a mixture of cells and α-DNP antibody without DNP-AF3.

The competitive α-DNP antibody binding molecule DNP-OH3 inhibited ternary complex formation in a concentration-dependent manner (FIG. 99). A sigmoidal concentration dependence on ternary complex formation inhibition was observed, with a calculated IC₅₀ of 40.6 nM. Because DNP-OH3 and DNP-AF3 share the same α-DNP antibody binding motif, the observation of half maximal inhibition of ternary complex formation would be expected when the two compounds are at equal concentrations. Indeed, it was observed that at concentrations approximately equal to DNP-AF3, DNP-OH3 inhibits approximately half of the total ternary complex formation.

It was then determined if competitive binders of the ASGPR protein impacted ternary complex formation. The small molecule AF is a synthetic sugar mimetic that binds to ASGPR more strongly than GalNAc. DNP-AF3 links together three AF sugars to bind strongly to ASGPR. The monomeric sugar AF was able to inhibit antibody association with cells at high concentrations, with an observed IC₅₀ of 1.45 μM (FIG. 100). These data are consistent with ternary complex formation between the α-DNP antibody and ASGPR on the surface of HepG2 cells.

It was also investigated whether proteins which have been reported to bind selectively to ASGPR impact ternary complex formation mediated by DNP-AF3 (FIG. 101). The serum protein ORM did not impact ternary complex formation. In contrast, desialylated ORM (ASOR) decreased ternary complex formation at high concentrations. In contrast to patterns observed with DNP-GN3, which showed complete inhibition of ternary complex at high concentrations of ASOR, 100% ternary complex formation inhibition was not reached. One possible explanation for this observation is that ASOR may not be present at high enough concentrations to effectively compete with DNP-AF3 for binding to ASGPR. Due to the solubility limits of the protein, a concentration of only 0.1 mg/mL (approximately 2.38 μM) was reached. Based on previous reports, it is expected that the AF3 molecule (which is present at 40 nM) binds more strongly to ASGPR than do both ASOR and DNP-GN3. Therefore, it may not be possible to reach a concentration of ASOR protein which effectively inhibits DNP-AF3 association with ASGPR.

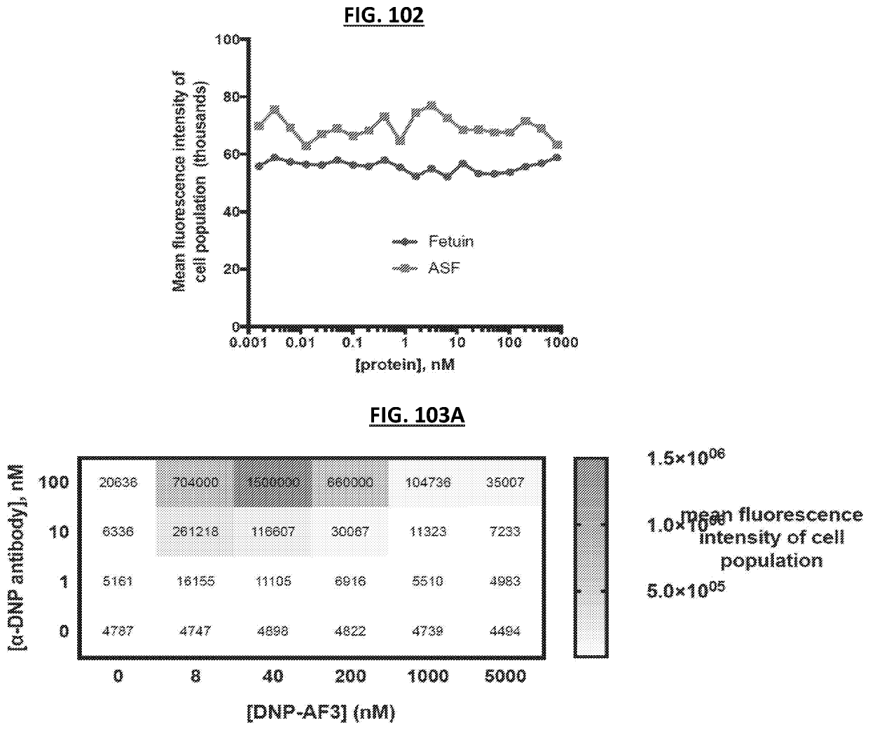

In contrast to the observations with DNP-GN3, the asialoglycoprotein ASF did not inhibit ternary complex formation mediated by DNP-AF3 (FIG. 102). ASOR binds to ASGPR with a Kd approximately 10-fold lower than ASF. At the concentrations of DNP-AF3 used in this experiment, ASF may not be present at high enough concentrations to significantly impact α-DNP antibody association with cells.

After establishing that DNP-AF3 is able to mediate the formation of a ternary complex between ASGPR present on the HepG2 cell surface and fluorescently labeled α-DNP antibody, next whether formation of this ternary complex resulted in α-DNP antibody endocytosis was investigated. The intracellular fluorescence of cells that were incubated at 37° C. with both fluorescently labeled α-DNP antibody and DNP-AF3 for a given amount of time was examined. Cells were then washed, removed from the plate with trypsin, and subjected to flow cytometry. Trypsin treatment is expected to cleave ASGPR and surface-bound α-DNP antibody from cells, and therefore the cellular fluorescence observed in these assays is expected to arise only from internalized antibodies.

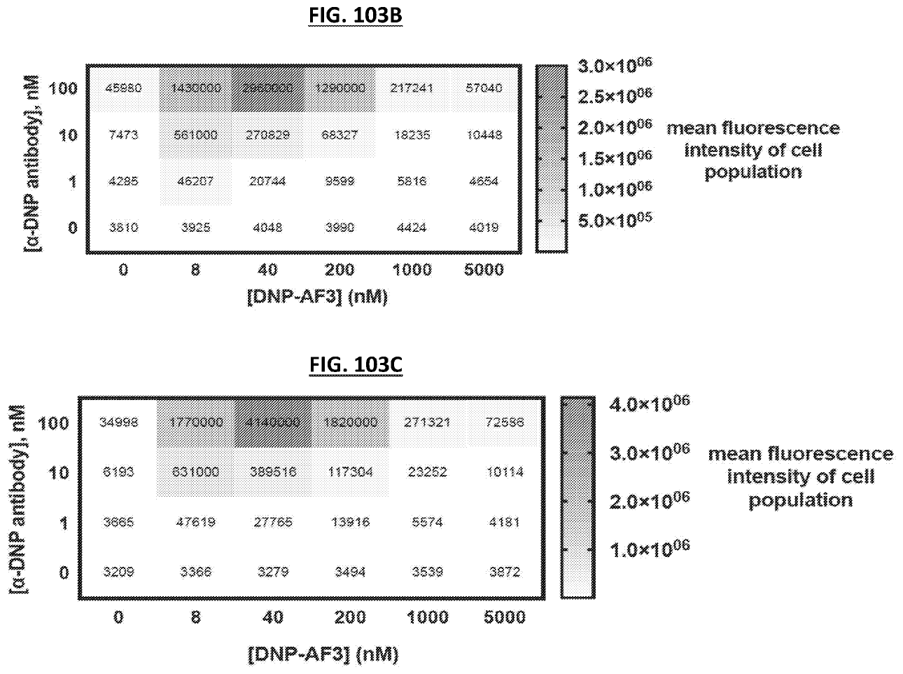

Intracellular HepG2 cell fluorescence was dependent on the concentration of both α-DNP antibody and DNP-AF3 (FIG. 103A). The greatest accumulation of intracellular fluorescence was observed at an α-DNP antibody concentration of 100 nM. Higher concentrations of antibody were not used because they led to drastically increased antibody endocytosis even in the absence of DNP-AF3 (data not shown). 40 nM DNP-AF3 resulted in maximal α-DNP antibody endocytosis when the target protein was present at a concentration of 100 nM. This is consistent with the ternary complex formation data, which demonstrated that 40 nM DNP-AF3 is the most effective at mediating association of α-DNP antibody with cells. In addition, endocytosis exhibited a bell-shaped dependence on DNP-AF3 concentration, and at micromolar levels of DNP-AF3, we observed near-background levels of endocytosis. These data support that formation of a ternary complex directly correlates with endocytosis. At decreased levels of α-DNP antibody (10 nM), maximal endocytosis was observed with DNP-AF3 at a concentration of 8 nM. This is consistent with ternary complex modeling, which predicts that, at decreased concentrations of target protein, lower concentrations of the adaptor molecule are required in order to mediate maximal ternary complex formation. Enhanced uptake of α-DNP antibody in the presence of DNP-AF3 was observed at concentrations as low as 1 nM α-DNP antibody. In contrast, the bifunctional molecule DNP-GN3 was not effective at mediating the uptake of α-DNP antibody when the target protein was present at a concentration of 1 nM. A time-dependent increase in intracellular fluorescence (FIGS. 103A and 103B) was also observed that reflected the trends observed at six hours. Together, these data indicate that DNP-AF3 mediates the endocytosis of α-DNP antibody across a wide range of concentrations, and that the molecule is more effective at mediating endocytosis than DNP-GN3.

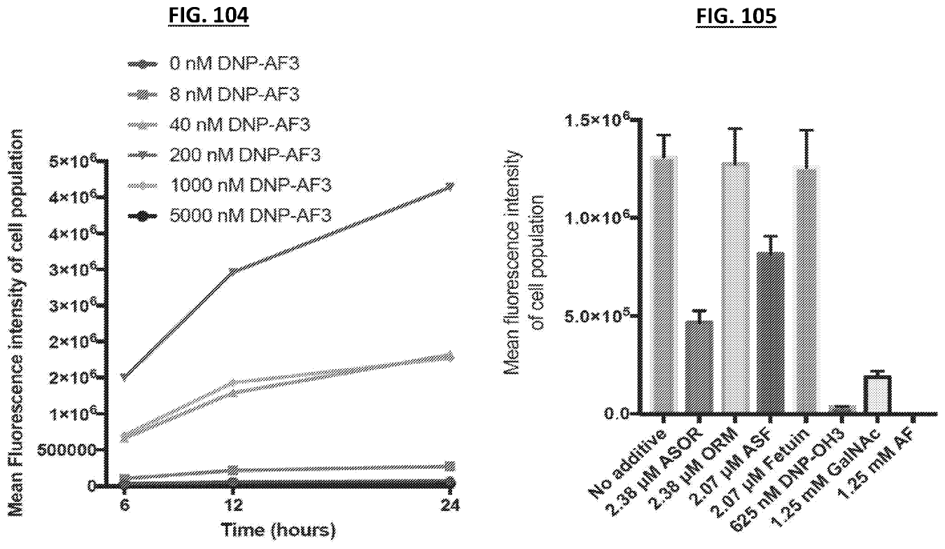

Alternatively, these data can be plotted to demonstrate the time-dependence of DNP-AF3-mediated α-DNP antibody endocytosis (FIG. 104). Over time, a concentration of 5 μM DNP-AF3 gives near-background levels of endocytosis. At concentrations of both 8 and 200 nM, DNP-AF3 mediates α-DNP antibody endocytosis over time. At a concentration of 40 nM, however, the strongest α-DNP antibody endocytosis over time was observed.

It was then determined if reagents which inhibit ternary complex formation inhibit endocytosis mediated by DNP-AF3. In order to account for non-specific antibody association with cells, the mean fluorescence intensity of an aliquot of cells which had been treated with α-DNP antibody but not DNP-AF3 was subtracted from each reading. In the absence of competitive inhibitor of endocytosis, the cell population had a mean fluorescence of approximately 1.31e6. In the presence of 2.38 μM (0.1 mg/mL) ASOR, the cell population fluorescence was decreased to 4.66e5 (FIG. 105); at this ASOR concentration, ternary complex formation is decreased by approximately 70%. Because ternary complex is not inhibited completely at this concentration of ASOR, it is expected that productive endocytic events are still taking place.

As discussed above, the asialoglycoprotein ASF was not effective at inhibiting ternary complex formation at any concentration of the protein tested. Although the protein was not effective at decreasing ternary complex formation, a significant decrease in endocytosis was observed after treatment of cells with ASF at a concentration of 2.07 μM. The serum proteins ORM and fetuin did not significantly impact fluorescent antibody endocytosis. Based on these data, which demonstrate that known proteins that bind to ASGPR inhibit DNP-AF3-mediated α-DNP antibody endocytosis, it was concluded that DNP-AF3 mediates endocytosis via ASGPR.

Cellular fluorescence was decreased to near-background levels by DNP-OH3, a competitive binder of the α-DNP antibody. DNP-OH3 was used at a concentration 15.6-fold greater than the IC₅₀ observed in ternary complex experiments. Monomeric sugars also decreased cellular fluorescence. When present at 1.25 mM—approximately three orders of magnitude greater than its observed IC₅₀ in ternary complex experiments—the monomeric AF sugar decreased cellular fluorescence to background levels. The monomeric GalNAc sugar, which has a lower affinity for ASGPR, also significantly decreased cellular fluorescence at a concentration of 1.25 mM. Based on these data, it was concluded that ternary complex formation is necessary for antibody endocytosis, and reagents which inhibit formation of a ternary complex also inhibit antibody endocytosis.

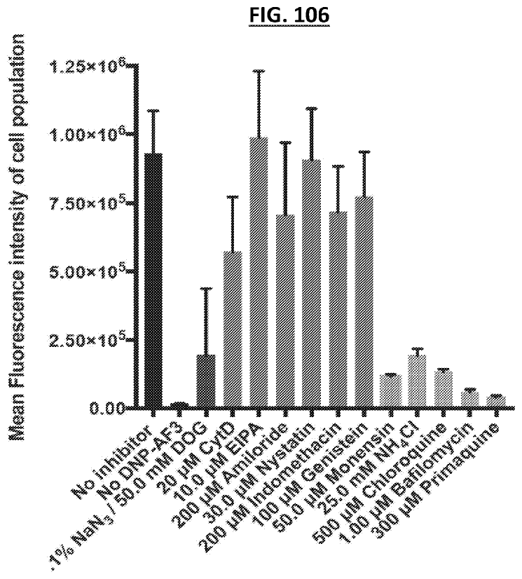

Next, whether inhibitors of specific endocytic pathways inhibited α-DNP antibody endocytosis was determined (FIG. 106). When incubated with both α-DNP antibody and DNP-AF3, the mean fluorescence intensity of the cell population was observed to be 9.31e6. In the absence of DNP-AF3, the mean fluorescence intensity of the cell population was reduced to 1.53e5. This supports a strong dependence on DNP-AF3 to mediate antibody uptake. When cells were treated with the metabolic poisons 2-deoxyglucose (DOG) and sodium azide, a significant decrease in the fluorescence of the cell population was observed. This is consistent with data gathered using DNP-GN3 and previous reports that treatment with sodium azide inhibits ASGPR recycling. One possible reason why these inhibitors do not cause complete abolition of uptake is because the concentrations here do not completely inhibit ATP-mediated endocytosis and receptor recycling. It is also possible that DNP-AF3 induces a non-ATP dependent mechanism of α-DNP antibody uptake.

Antibody endocytosis was significantly inhibited by some inhibitors of macropinocytosis and phagocytosis. Treatment with cytochalisin D (CytD) gave a non-significant decrease in the fluorescence intensity of the cell population. One possible explanation for this is that CytD inhibits processes responsible for trafficking of endosomes throughout the cell, and disruption of those networks could decrease ASGPR recycling. A decrease in cellular fluorescence with the macropinocytosis inhibitor amiloride was seen, but not with the closely related compound EIPA. It is also possible that some of the inhibitors tested in these assays are not specific for their prescribed pathway, but rather have effects on several different endocytic pathways. Treatment with the caveolin-dependent endocytosis inhibitors nystatin, indomethacin, and genistein did not significantly decrease fluorescence of the cell population. In contrast, all tested inhibitors of clathrin-mediated endocytosis significantly decreased antibody endocytosis compared to uninhibited cells. These inhibitors are either weak bases that neutralize endosomes and lysosomes or inhibit proton pumps which acidify endolysosomal compartments. Based on these data, it was concluded that DNP-AF3 mediates α-DNP antibody endocytosis consistent with ASGPR targeting. A fraction of the endocytosis mediated by DNP-AF3 may occur through non-clathrin-dependent pathways.

### Example 30: DNP-AF3 Mediates the Degradation of α-DNP Antibody

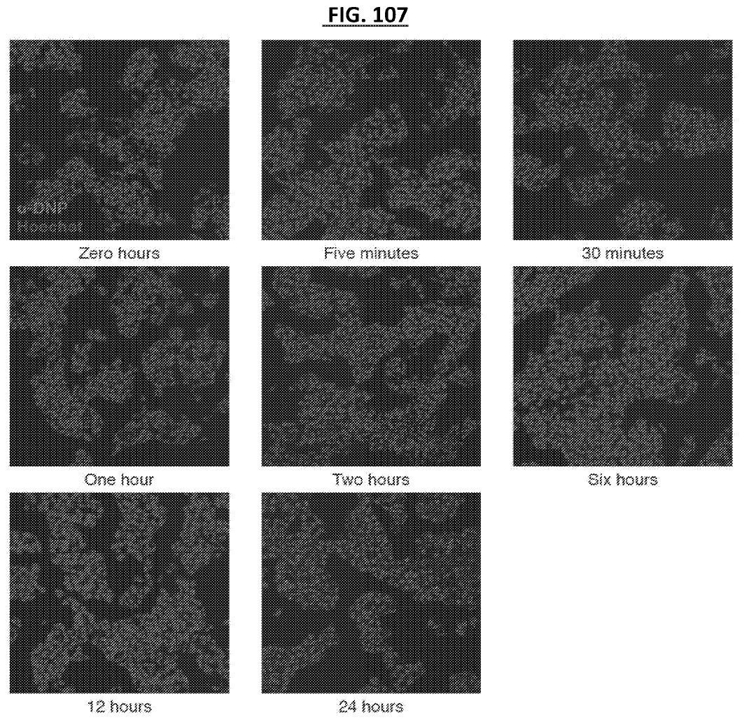

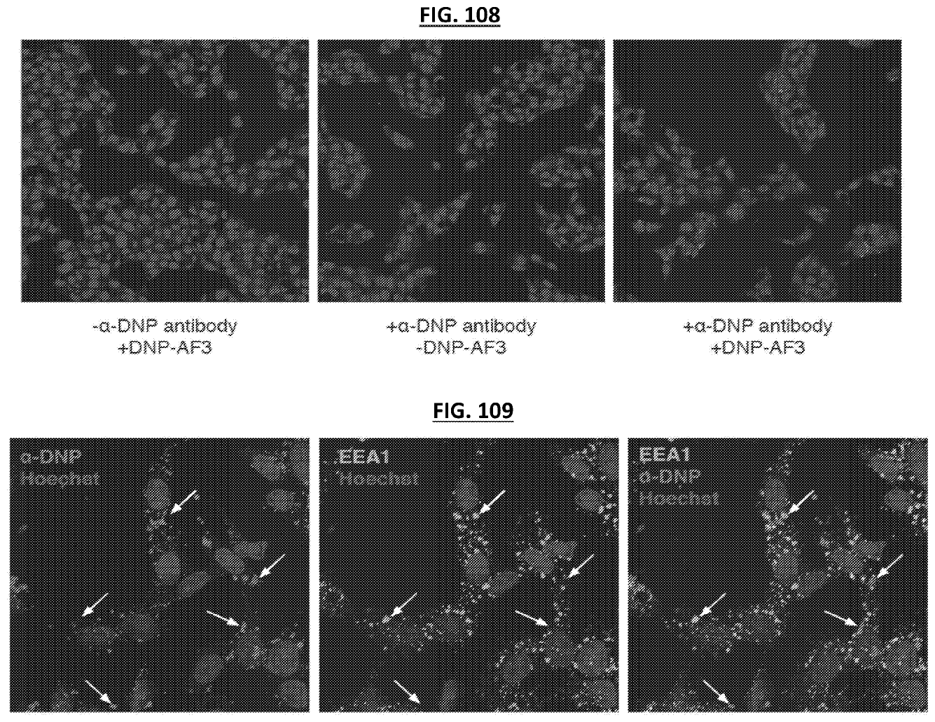

In order to investigate the intracellular localization endocytosed of α-DNP antibody, colocalization experiments were undertaken using antibodies directed to makers of different cytoplasmic compartments. Punctae containing endocytosed Alexa 568-labeled α-DNP antibody were found to accumulate in cells over time, with distinct punctae present following one hour of incubation with α-DNP antibody and DNP-AF3 (FIG. 107). The abundance and brightness of punctae increased gradually over the 24-hour time course of this experiment. For further cell experiments, a 12-hour time point was used for analysis. In addition, it was determined whether intracellular fluorescence arising in the Alexa 568 fluorescence channel was dependent on the presence of both Alexa Fluor 568-labeled α-DNP antibody and DNP-AF3. Cells were incubated with or without each reagent for 12 hours, then fixed and imaged. Bright intracellular Alexa-568 punctae were observed only in cells treated with both α-DNP antibody and DNP-AF3 (FIG. 108).

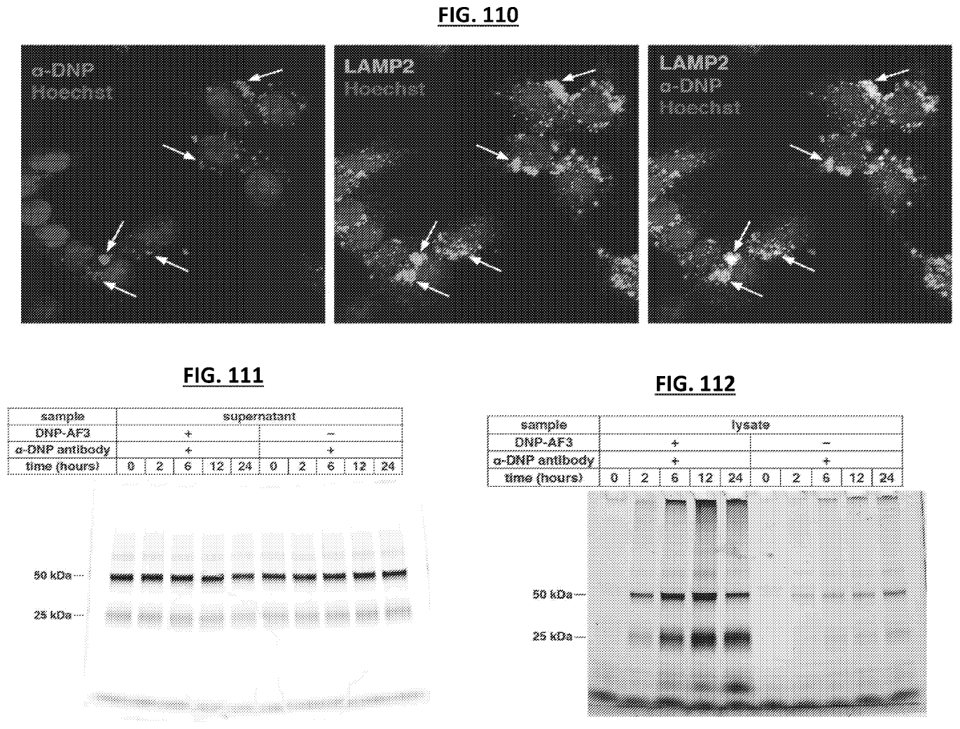

Then, whether fluorescence derived from endocytosed α-DNP antibody colocalized with early or late endosomes in cells was investigated. It was found that endocytosed α-DNP antibody did not colocalize strongly with an antibody that recognizes the early endosome protein EEA1, indicating that endocytosed antibody does not accumulate in early endosomes (FIG. 109). In contrast, α-DNP antibody-derived fluorescence colocalized strongly with the protein LAMP2, which is present in the membrane of late endosomes and lysosomes (FIG. 110). Based on these observations, it was concluded that the α-DNP antibody is trafficked to late endosomes and lysosomes in cells, and that by 12 hours the majority of antibody is present in mature endocytic compartments in the cell. After establishing that DNP-AF3 mediates the endocytosis of α-DNP antibody and that antibody-derived fluorescence colocalizes with the late endosome marker LAMP2 in cells, whether endocytosed α-DNP antibody is degraded in cells was determined. In order to do this, cells were treated with Alexa-568 labeled α-DNP antibody and DNP-AF3 for a given amount of time, then lysed in the presence of protease inhibitors. Several different visualization methods and polyacrylamide gel electrophoresis (PAGE) conditions were investigated with varying levels of success.

First, reducing conditions were used to visualize antibody degradation via PAGE gel. Under these conditions, strong bands in cell culture supernatant with molecular weights of 50 and 25 kDa were observed (FIG. 111). Although not wishing to be limited by theory, it was hypothesized that these bands correspond to the heavy and light chains of the α-DNP antibody, respectively. The levels of α-DNP antibody observed in cell supernatants did not change over time in the presence or absence of DNP-AF3. In addition to the expected bands at approximately 25 and 50 kDa, molecular weight fluorophore-associated protein fragments were observed at a molecular weight of approximately 75 kDa. Although their identity has not been investigated, these protein bands could represent aggregated forms of the α-DNP antibody or a contaminating protein present at low levels in the commercial antibody stock solution. In addition, fluorescence signal was observed very near to the loading dye front. One possibility is that these bands are hydrolyzed Alexa 488 fluorophore present in the commercial antibody solution. Neither the presence of DNP-AF3 nor the time at which samples collected changed the brightness of these bands compared to the bands present at 50 and 25 kDa.

Antibody accumulation in cell lysates showed a dependence both on time and on the presence of DNP-AF3 (FIG. 112). In the absence of DNP-AF3, a small amount of α-DNP antibody was endocytosed by cells, consistent with flow data showing that there is a low level of α-DNP antibody uptake even in the absence of DNP-AF3. This background α-DNP antibody uptake may be due to nonspecific endocytosis of the α-DNP antibody, or by binding of the α-DNP antibody to a cell-surface receptor (for example, FcRN) which mediates its endocytosis and accumulation in cells. In the presence of DNP-AF3, strong antibody uptake by 2 hours was observed, with the amount of intracellular fluorescence increasing at each further time point. In addition to the expected bands at 50 and 25 kDa, signal arising from the band slightly 50 kDa was observed, which was also observed in cell supernatants. In addition to this protein fragment, the low molecular weight fluorescent signal that traveled near the dye front was again observed.

In cell lysates, two fluorophore-associated protein fragments were observed that were not observed in cell supernatants. The first was found in the well of the gel, which is hypothesized to represent higher molecular weight aggregates of the α-DNP antibody in cell lysates. In addition, a fluorescent band below 10 kDa was observed that was found in the lysates of cells treated with DNP-AF3 after a six hour incubation. Because this band is strongly observed only in cell lysates and only in cells treated with DNP-AF3, it was hypothesized that this is a lower molecular weight protein fragment derived from lysosomal proteolysis of the α-DNP antibody.

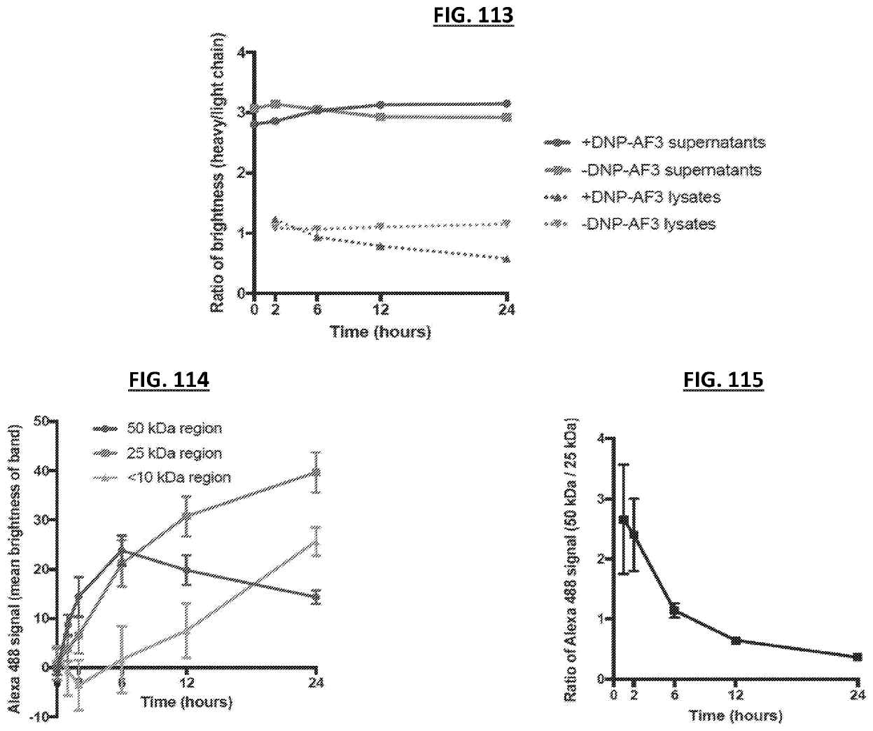

In addition to observing increasing amounts of α-DNP antibody in cell lysates, a change in the abundance of each protein fragment was observed relative to the other protein fragments. In the cell supernatant samples, the brightness of the heavy chain was approximately 3-fold brighter than the light chain under all conditions (FIG. 113). In the lysates of DNP-AF3 treated cells, however, while at 2 hours the heavy and light chains were approximately equal in brightness, the intensity of fluorophore signal arising from proteins with molecular weights of 25 kDa increases over time. By 24 hours, the ratio intensity of fluorescence arising from 50 kDa fragments compared to 25 kDa fragments is 0.58, compared to 3.15 for the supernatants of those cells. Given this data, it was hypothesized that the 50 kDa band is proteolyzed over time into two smaller fragments of molecular weight 25 kDa each. These fragments overlap with the 25 kDa light chain and result in an increase of fluorescence at that molecular weight compared to 50 kDa. In addition, it was hypothesized that one or both of the antibody chains are proteolyzed to produce the lower molecular weight band (<10 kDa) observed in cell lysates. In the lysates of cells treated only with α-DNP antibody, the heavy chain was approximately equally bright as the light chain at all time points. It was hypothesized that that this is due to a low basal level of proteolysis of the endocytosed 50 kDa heavy chain.

The change in the intensity of individual protein bands was observed across many cell lysates. The intensity of fluorescence associated with proteins of 50 kDa molecular weight increased at time points up to six hours, after which a gradual degrease in their fluorescence was observed (FIG. 114). In contrast, the intensity of the protein fragments of molecular weight 25 kDa increased over all time points. The band at less than 10 kDa became noticeable over background levels after approximately 12 hours of incubation with both DNP-AF3 and α-DNP antibody, and by 24 hours was the second brightest band present in the cell lysate. Collectively, these data indicate that α-DNP antibody is endocytosed over time, and that α-DNP antibody-derived fluorescence is associated with lower molecular weight fragments over time.

It was hypothesized that these observations are due to the proteolysis of endocytosed antibody by lysosomal proteases. For example, it was hypothesized that the buildup in protein fragments with a weight of 25 kDa is due to proteolysis of the heavy chain antibody protein. It was also hypothesized that the fragments observed at <10 kDa are proteolysis products of both the 50 and 25 kDa bands. A ratiometric representation of the intensity of the fluorescent signal observed associated with proteins of molecular weight 50 kDa divided by proteins of molecular weight 25 kDa is presented in FIG. 115.

The accumulation of fluorescently labeled protein fragments at both 25 kDa and <10 kDa was inhibited by the addition of several protease inhibitors. The ratiometric comparison of band intensity at 50 kDa divided by the intensity of the 25 kDa was used as a measure of α-DNP antibody degradation. As endocytosed α-DNP antibody is degraded in cells, this ratio decreases. By analyzing data in this way, the endocytosis of α-DNP antibody did not need to be controlled for in order to determine whether protease inhibitors were effective. Each ratiometric measurement is produced using only the protein fragments present in the cell lysate.

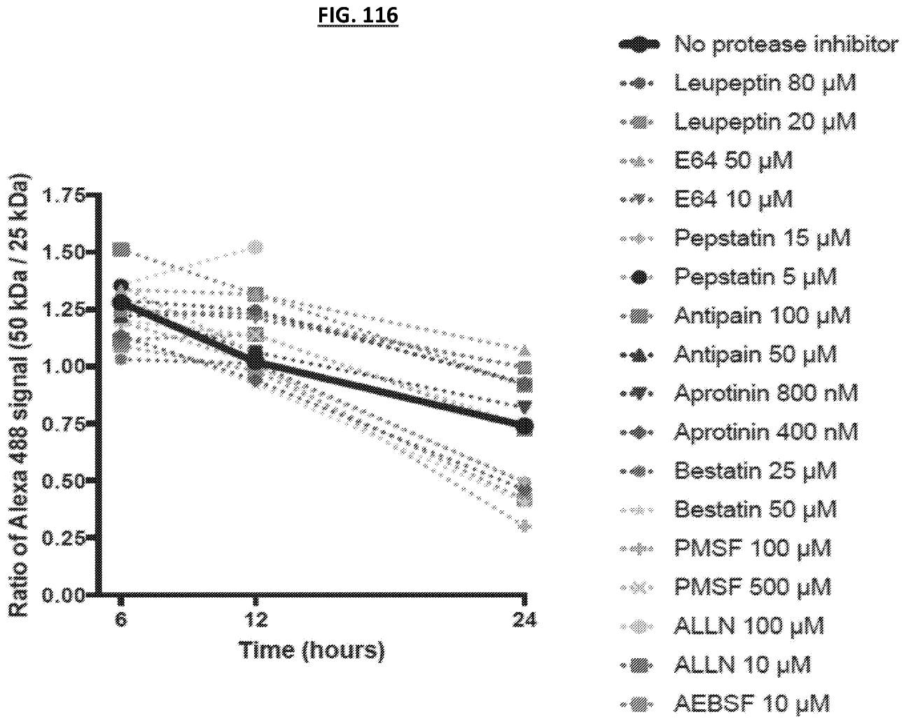

In the absence of protease inhibitors, a general decrease was observed in the ratio of 50 kDa- and 25 kDa-chain derived signal, with ratios of 1.28, 1.02, and 0.74 observed at six, 12, and 24 hours respectively (FIG. 116). Protease inhibitors which inhibit α-DNP antibody degradation are expected to cause an accumulation of fluorescent intensity at 50 kDa and a decrease in fluorescent intensity at 25 kDa. Therefore, effective inhibitors would exhibit intensity ratios greater than the ratios observed for cells not treated with protease inhibitors. Several inhibitors proved active at decreasing α-DNP antibody degradation.

The protease inhibitor leupeptin was effective at inhibiting α-DNP antibody degradation at both 20 and 80 μM. Leupeptin is an aldehyde-containing tripeptide that forms covalent bonds with active site residues of both serine and cysteine proteases, and has previously been used successfully in HepG2 cells to inhibit lysosomal degradation of proteins. E64 is a covalent inhibitor of cysteine proteases contains a trans-epoxysuccinyl group that has been effectively used in HepG2 cells to inhibit protein degradation. Unlike leupeptin and antipain, E64 is specific for cysteine proteases such as papain, actinidase, and cathepsins B, H, and L. E64 at concentrations of both 50 and 10 μM was effective at decreasing anti-DNP antibody degradation. Pepstatin is an inhibitor of aspartic proteases that has previously been shown to inhibit protein degradation in HepG2 cells. Pepstatin was not effective at decreasing antibody degradation at any time point. After 24 hours, a concentration of 5 μM pepstatin was toxic to cells. Antipain is an oligopeptide which inhibits both cysteine and serine proteases by forming a covalent bond with protease active site nucleophilic residues. Antipain was effective at inhibiting α-DNP antibody degradation at both 50 and 100 μM. Aprotinin is a 58-mer protein which inhibits serine proteases, and has been used successful to inhibit protein degradation in HepG2 cells. Aprotinin was not effective at inhibiting α-DNP antibody degradation at a concentration of either 400 or 800 nM. While this protein is reported to be cell-permeable, its proteinaceous character may mean that it is degraded by other proteases in the lysosome, or that aprotinin localizes to the cytosol rather than the lysosome. Bestatin is an inhibitor of the amino proteases, such as leucine aminopeptidase and aminopeptidase N. These proteases are responsible for cleaving single N-terminal amino acids from protein chains. Bestatin was not effective at inhibiting α-DNP degradation at either concentration tested. Because bestatin inhibits proteases that catalyze the removal of only a single amino acid from proteins, any degradation inhibition may not be significant enough to change the migration of fluorescently labeled protein fragments. Phenylmethylsulfonyl fluoride (PMSF) is a small molecule which covalently inhibits serine proteases, and has been used effectively in HepG2 cells. PMSF was not effective at inhibiting α-DNP antibody degradation at a concentration of either 100 or 500 μM. PMSF has been reported to be very unstable in aqueous solutions and to have poor solubility, so it is possible that this inhibitor was either inactivated in solution or at a very low concentration in cell culture supernatant. 4-(2-aminoethyl)benzeneulfonyl fluoride hydrochloride (AEBSF) is similar in structure to PMSF, but is reported to be more stable at lower pH values. The sulfonyl chloride group reacts with active site nucleophiles of proteases. AEBSF was not effective at decreasing α-DNP antibody degradation at 10 μM. At 100 μM, AEBSF was toxic to cells. Calpain Inhibitor I (Ac-LLnL-CHO, ALLN) covalently inhibits both serine and cysteine proteases. ALLN is similar to leupeptin, but may be more cell permeable because it is more hydrophobic. At 10 μM, ALLN was not active at inhibiting anti-DNP antibody degradation. At 100 μM, however, ALLN showed strong inhibition at the 12 hour time point. By 24 hours, 100 μM ALLN was observed to be toxic to cells.

After carrying out the initial screening for protease inhibitors which decrease α-DNP antibody degradation, a single time point (12 hours) was elected for future studies. The 24 hour time point was not chosen because at that time, most cell lysates showed a more drastic increase in low molecular weight protein fragments even in the presence of effective protease inhibitors. This is perhaps due to the protease inhibitors becoming hydrolyzed or otherwise inactivated in solution. This observation may also be due to the production of more proteases to compensate for the covalently inhibited proteases present in cells.

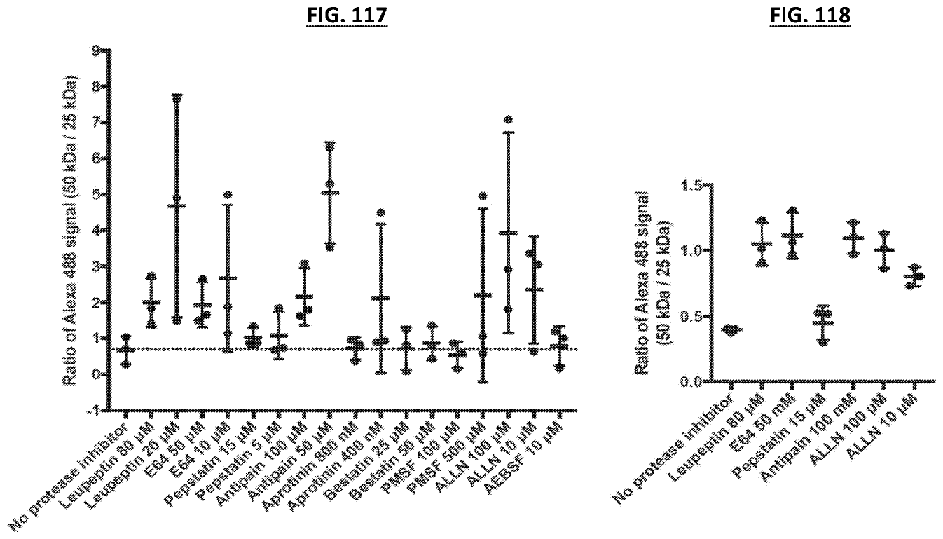

Next it was studied whether the inhibition of α-DNP antibody degradation by protease inhibitors was reproducible. Each protease inhibitor concentration was tested in triplicate in HepG2 cells (FIG. 117). Leupeptin, E64, and antipain were shown to inhibit α-DNP antibody degradation at all concentrations tested, consistent with the screening results. Expectedly, pepstatin, aprotinin, bestatin, PMSF, and AEBSF did not significantly inhibit α-DNP antibody degradation. ALLN appeared effective at inhibiting anti-DNP antibody degradation at both 100 and 10 μM in this assay, although the observed degradation inhibition varied widely between replicates. No visual difference in cell morphology or adherence was observed at the 12-hour time point for either concentration of ALLN.

Based on these data, a further repeat of this experiment was undertaken using only 5 conditions which showed the most robust inhibition of α-DNP antibody degradation. Pepstatin at a concentration 15 μM was included as a negative control. Expectedly, 80 μM leupeptin, 50 mM E64, 100 mM antipain, and both 10 and 100 μM ALLN inhibited degradation of α-DNP antibody in cells, while pepstatin did not (FIG. 118). The degradation inhibition observed in cells treated with 80 μM leupeptin was not significant (p=0.0504).

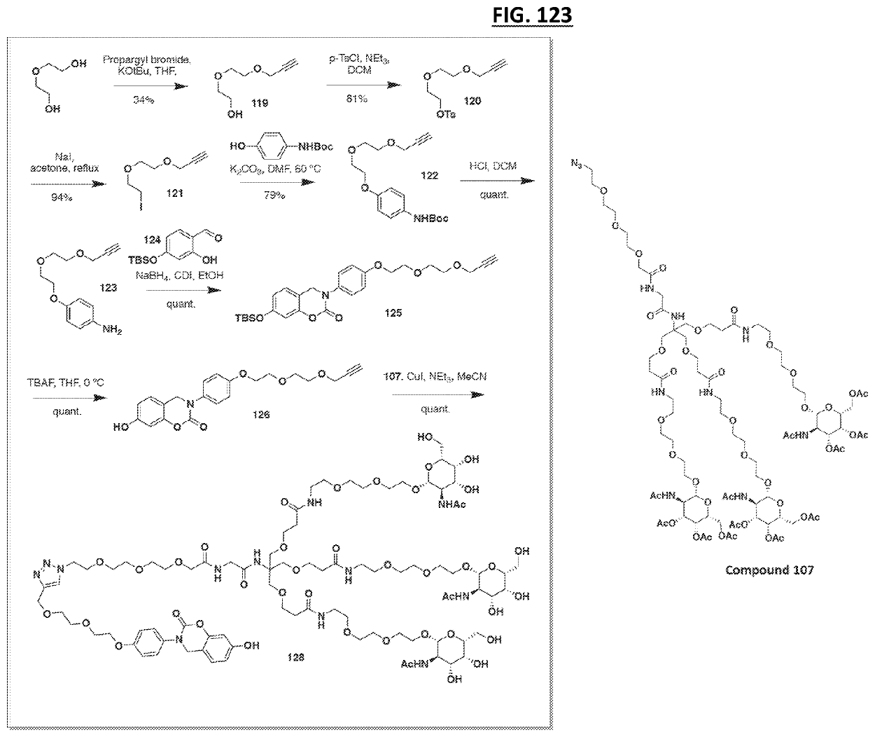

### Example 31: Synthesis of MIF-GN3 (FIGS. 123A-123B)

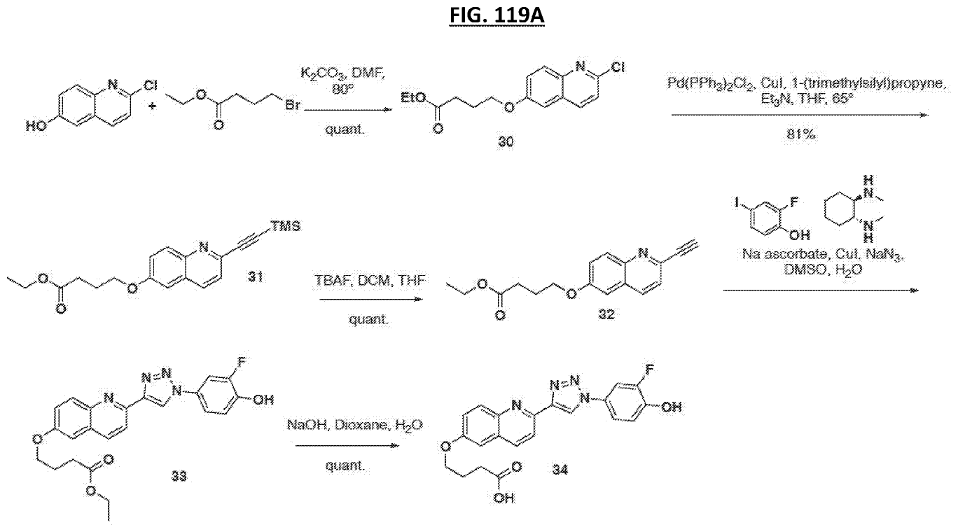

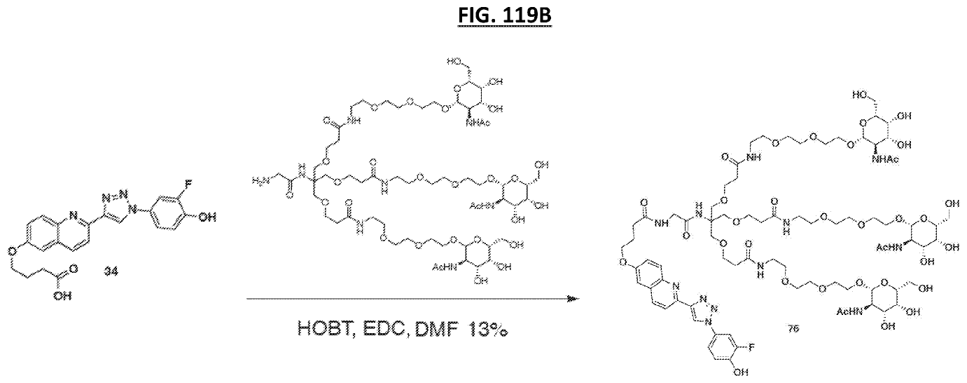

First, the carboxylic acid-terminated MIF inhibitor 34 was synthesized (FIG. 1I9A). Reaction of 2-chloroquinolin-6-ol and ethyl bromobutyrate in the presence of a weak base afforded ethyl ester 30, which was then subjected to Sonogashira coupling conditions to produce the TMS-protected alkyne 31. Deprotection yielded the alkyne 32, which then underwent a click reaction to generate the triaryl MIF inhibitor 33. Treatment with sodium hydroxide afforded the carboxylic acid 34. The final bifunctional molecule MIF-GN3 (76) was formed through EDC-mediated amide bond formation with the tri-GalNAc ASGPR ligand 74 (FIG. 119B). Following HPLC purification, compound 76 was recovered in 13% yield.

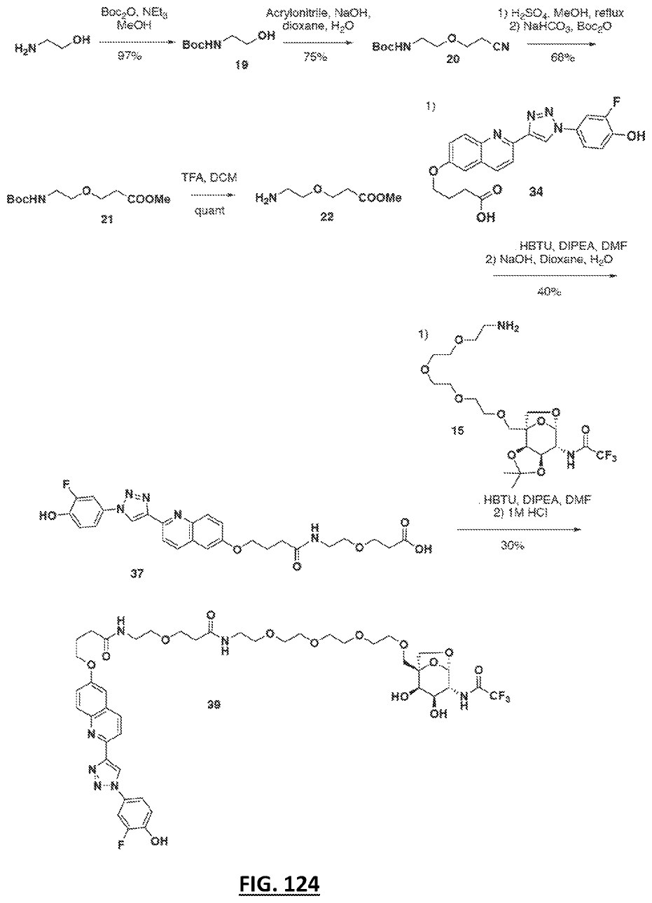

### Example 32: Synthesis of MIF Inhibitor 3w (FIG. 124)

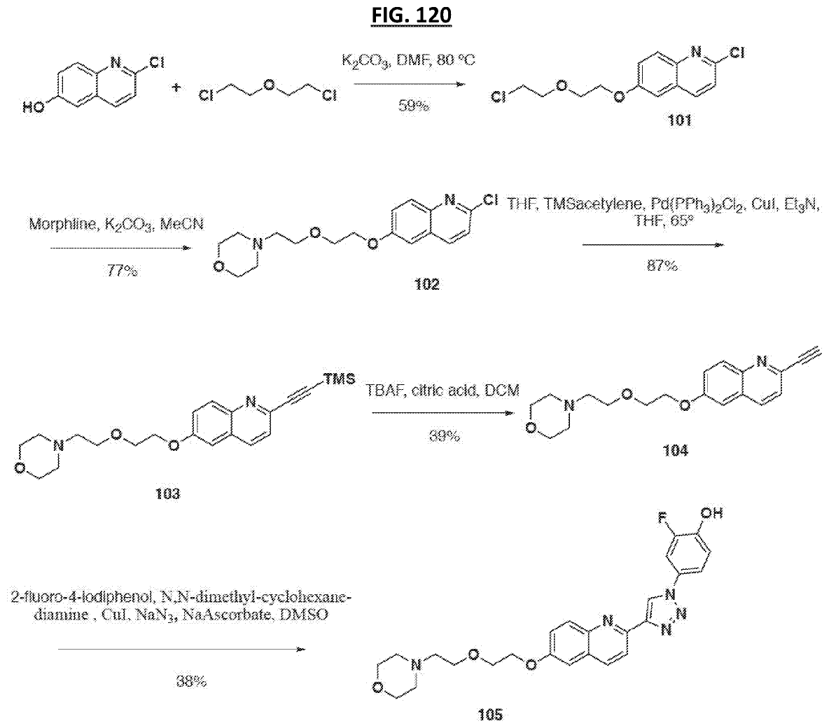

An inhibitor of MIF's enzymatic activity was synthesized for use as a negative control compounds in protein depletion experiments. The morpholine-terminated MIF inhibitor 3w (105) was synthesized through adaptation of the procedure described above to synthesize carboxylic acid 34. The morpholine group was installed early in the synthesis through nucleophilic substitution of the terminal chloride of compound 101 (FIG. 120). Compound 105 was used in both enzyme inhibition assays as well as in vitro and in vivo disease state efficacy assays.

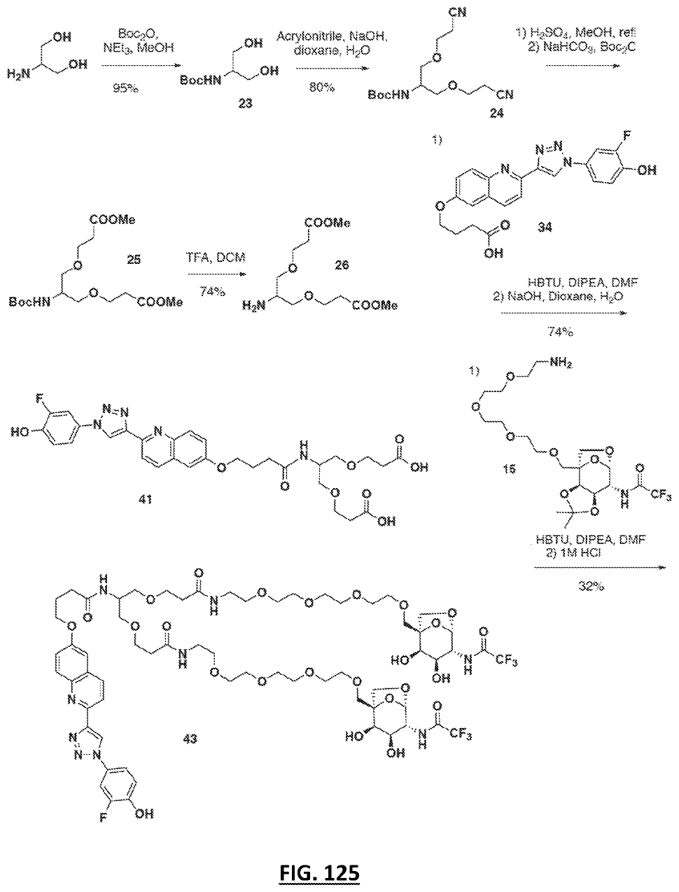

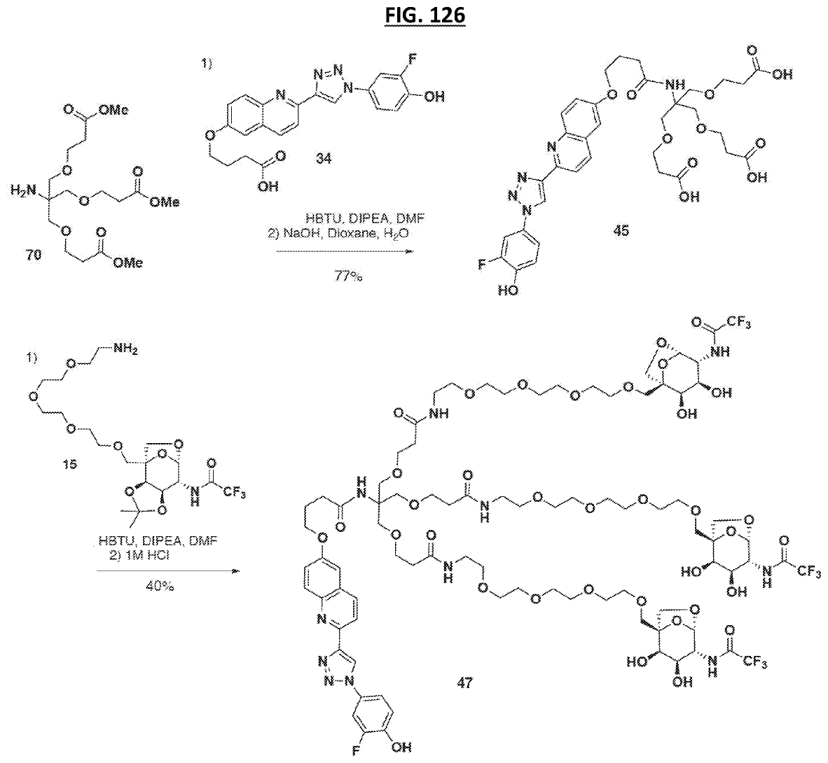

### Example 33: Synthesis of MIF-PEG2-GN3 (FIG. 125) and MIF-PEG4-GN3 (FIG. 126)

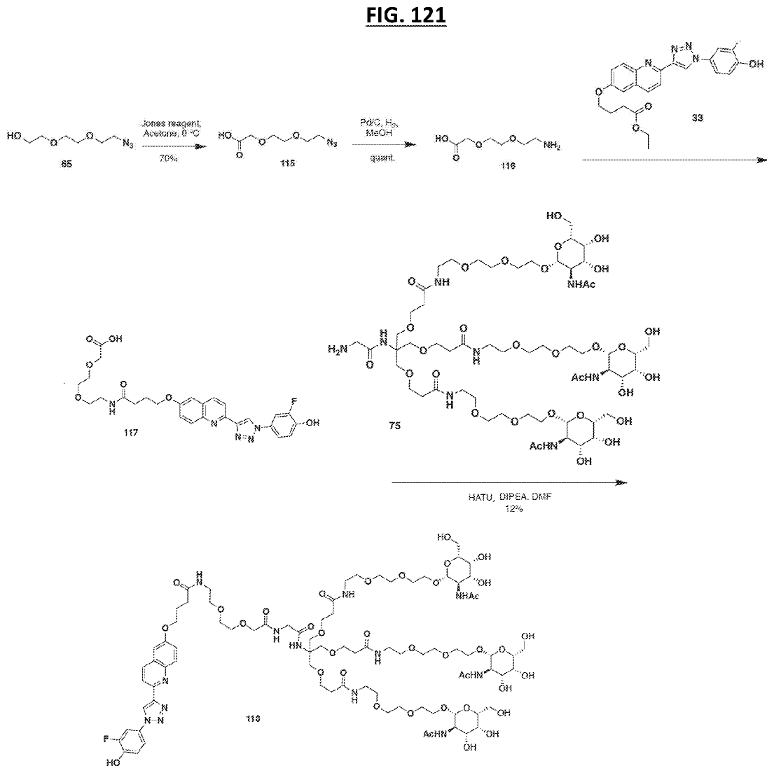

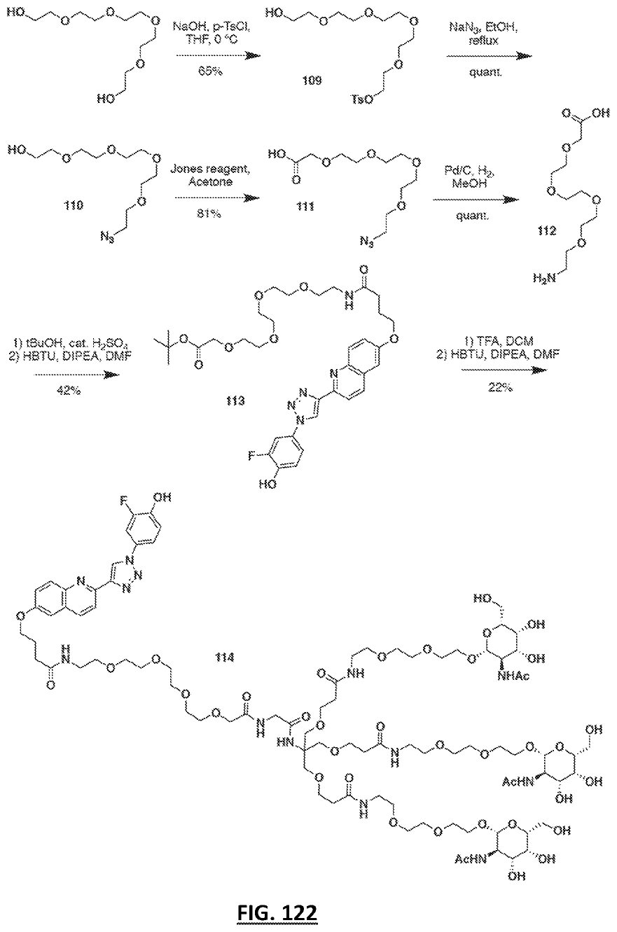

Versions of the MIF-GN3 (76) bifunctional molecule with additional PEG spacers between the tri-GalNAc targeting motif and the MIF-binding moiety were synthesized. MIF-PEG2-GN3 (FIG. 121) and MIF-PEG4-GN3 (FIG. 122) were synthesized by modifying the MIF inhibitor 34 with extended PEG linkers before conjugation to tri-GalNAc molecule 75.

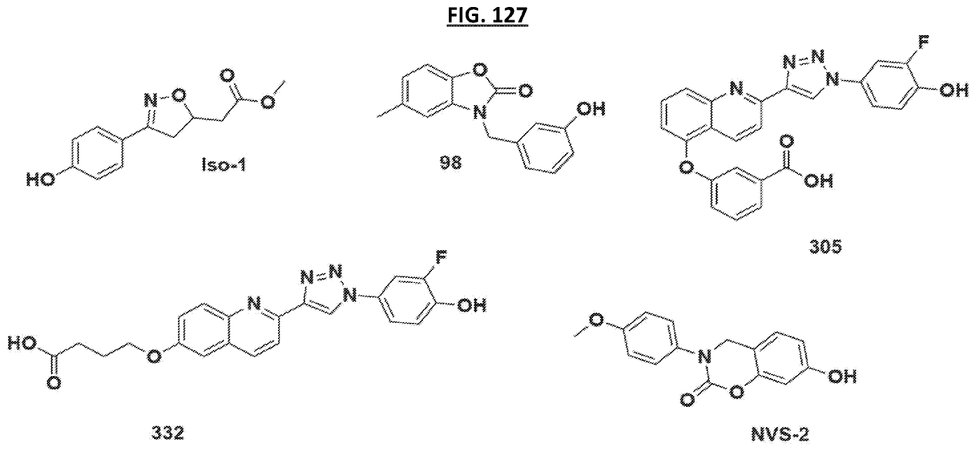

### Example 34: Synthesis of MIF-NVS-PEG3-GN3 (FIG. 127)

A bifunctional molecule that binds to MIF was synthesized by incorporation of a MIF inhibitor that is structurally dissimilar from that utilized in the bifunctional molecule MIF-GN3. The azido-terminated MIF inhibitor was conjugated with the tri-GalNAc motif through copper-mediated triazole formation to afford final compound MIF-NVS-PEG3-GN3 (FIG. 123).

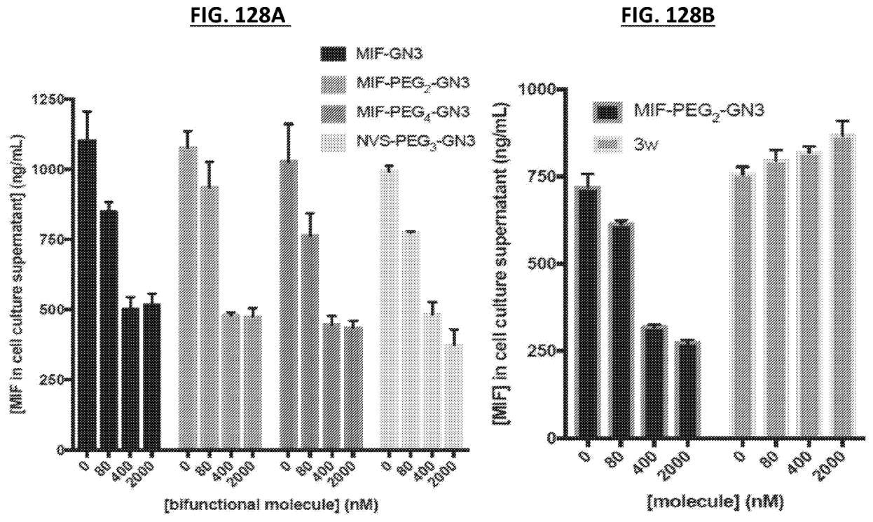

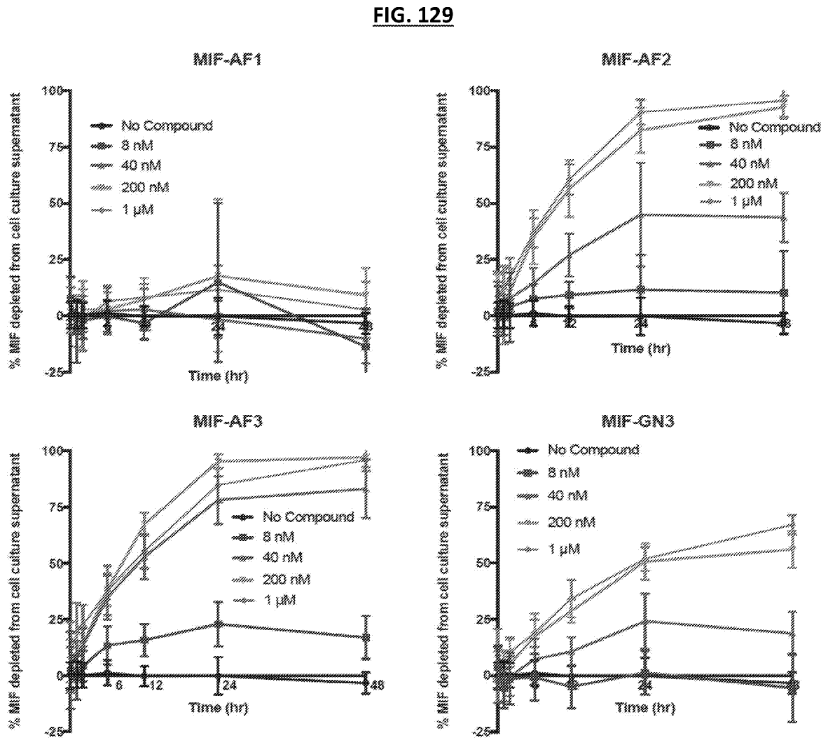

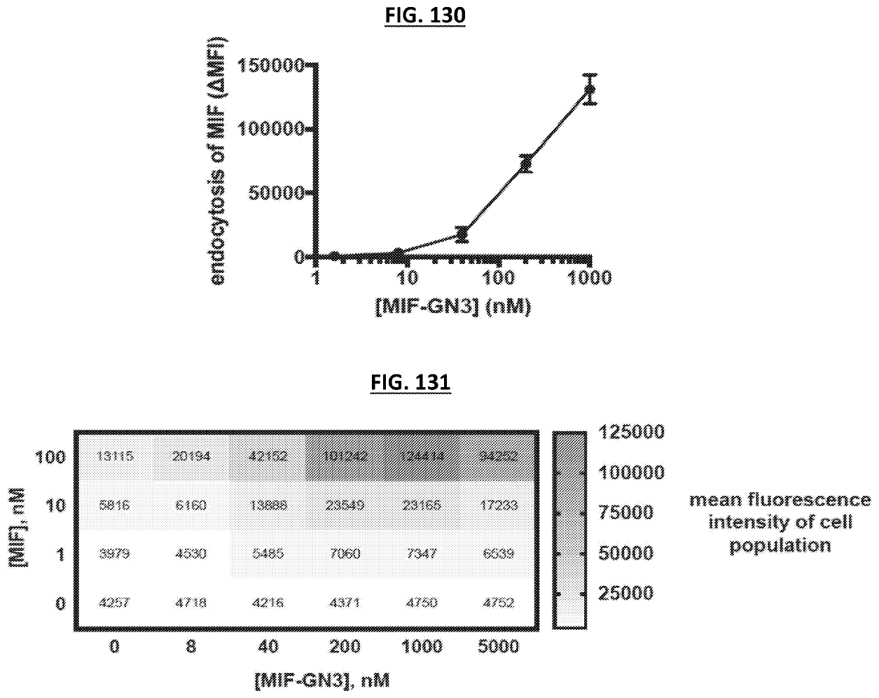

### Example 35: Synthesis of MIF-AF1 (FIG. 128), MIF-AF2 (FIG. 129), and MIF-AF3 (FIG. 130)

Molecules which incorporated the high-affinity ASGPR ligand 15 to degrade MIF were synthesized. In addition, the impact of sugar valency on the ability of these molecules to degrade the MIF protein was explored. Through adaptation of previously discussed procedures, the bifunctional molecules MIF-AF1 (39, monovalent display of sugar 15, FIG. 124), MIF-AF2 (43, divalent display, FIG. 125), and MIF-AF3 (47, trivalent display, FIG. 126) were synthesized.

### Example 36: Reported Inhibitors of MIF's Enzymatic Activity are Active Against Mouse MIF

Although numerous inhibitors of MIF's enzymatic activity have been reported, these small molecules have only been assayed against the human MIF protein. Therefore, whether reported MIF inhibitors were also effective at inhibiting the tautomerase reaction carried out by mouse MIF was studied. It was hypothesized that inhibitors found to inhibit both human and mouse MIF could be elaborated into bifunctional molecules with the ability to degrade MIF protein from both species. The primary sequence of MIF is highly conserved across rodents and mammals, with greater than 90% sequence conservation between species.

In order to determine whether reported inhibitors bind to mouse MIF, enzymatic activity assays were undertaken to measure the impact of these small molecules on mouse MIF's enzymatic activity. Both human MIF and mouse MIF were found to mediate the tautomerization of D-dopachrome. For all commercial preparations assayed, mouse MIF carried out the tautomerization reaction more slowly than human MIF when present at the same concentration. Under the assay conditions used, human MIF protein carried out complete tautomerization of its substrate in less than ten minutes. In contrast, the mouse MIF assays required up to 20 minutes to reach completion. The relative catalytic rates of human versus mouse MIF have not been investigated. One possible reason for the decreased enzymatic rate observed for mouse MIF could be the inefficient posttranslational processing of the protein. Post-translational cleavage of the N-terminal methionine residue from both human MIF and mouse MIF is necessary for the protein's enzymatic activity, in recombinant preparations, this modification is oftentimes not carried out. If the mouse MIF protein was not post-translationally modified to the same extent as the human MIF protein, a much smaller proportion of the protein would be expected to be enzymatically active. This would be consistent with the observed decrease in tautomerase rate. Several MIF inhibitors were assayed for their ability to inhibit mouse MIF's tautomerase activity (FIG. 127).

### Example 37: Bifunctional Molecules Mediate the Endocytosis of Human MIF

The estimations of circulating MIF levels in humans range widely, from less than 1 ng/mL (80 μM) in healthy patients to up to 300 ng/mL (24 nM) in certain disease states. The levels of circulating MIF in mice range from 60-140 ng/mL.

The ability of bifunctional MIF-binding molecules to mediate the depletion of human MIF from cell culture was investigated. In these assays, human MIF was present at a concentration of 100 nM, and a sandwich ELISA assay was utilized to measure the concentration of human MIF remaining in the cell culture supernatant. The closely related bifunctional molecules MIF-GN3, MIF-PEG2-GN3, and MIF-PEG4-GN3 were used, as well as the structurally dissimilar molecule MIF-NVS-PEG3-GN3. After an incubation of 24 hours, all bifunctional molecules tested were effective at mediating the depletion of human MIF from cell culture supernatant (FIG. 128A). The most effective concentrations for each bifunctional molecule were found to be 400 and 2000 nM. Based on the ease of its synthesis and its efficacy in this experiment, MIF-GN3 was utilized for further studies. It was next investigated whether the MIF inhibitor 3w mediates MIF depletion from cell culture supernatant. 3w is not expected to mediate target protein degradation because it does not engage ASGPR to mediate MIF's endocytosis and degradation. When cells were treated with 3w, no depletion of exogenous MIF protein was observed after 24 hours (FIG. 128B). In contrast, the bifunctional molecule MIF-PEG2-GN3, which shares the same MIF-binding motif as 3w, mediated MIF depletion across a range of concentrations. From these data, it was concluded that in order to mediate the depletion of MIF from supernatant, a molecule must engage both MIF and ASGPR.

Bifunctional molecules containing optimized ASGPR-binding motifs are also capable of mediating MIF depletion from cell culture supernatant (FIG. 129). The MIF-binding molecule MIF-AF1, which contains only a single ASGPR-binding sugar, was not effective at mediating depletion of MIF from supernatant at any concentration tested. It is hypothesized that this is due to the compound's low predicted affinity for ASGPR (μM range). Because it only displays a single sugar residue, the affinity of this molecule for ASGPR is not enhanced by avidity effects. In contrast, the divalent ASGPR-binding molecule MIF-AF2 was effective at depleting MIF from cell culture supernatant at concentrations of 40 nM, 200 nM, and 1 μM. At a concentration of either 200 nM or 1 μM, MIF-AF2 mediated nearly 100%/o depletion of MLF from the cell culture supernatant after 48 hours. Similar results were observed with the trivalent ASGPR-binding molecule MIF-AF3. In contrast to these molecules, which utilize optimized synthetic ASGPR ligands, the GalNAc-based molecule MIF-GN3 mediated only 67.0% and 56.0% removal of MIF from supernatant after 48 hours at concentrations of 1 μM and 200 nM, respectively. Based on these data, it was concluded that bifunctional molecules which utilize optimized ASGPR-binding sugars may be more effective at mediating target protein depletion from supernatant. In addition to its observed efficacy, the MIF-AF3 compound demonstrates evidence of catalytic activity in preliminary assays. At a concentration of 40 nM (4.0 pmol of molecule), MIF-AF3 mediates the removal of 83.2% of MIF present in the cell culture supernatant, or 8.32 pmol of human MIF protein. Therefore, it was concluded that under the conditions of this experiment, MIF-AF3 mediates the depletion of 2.08 molar equivalents of MIF protein.

After demonstrating that MIF-binding bifunctional molecules can mediate the depletion of MIF from cell culture supernatant, whether endocytosed MIF protein accumulates in cells was determined. Human MIF protein was fluorescently labeled with Alexa 488 NHS ester, then incubated with HepG2 cells in the presence of varying levels of MIF-GN3. Increased intracellular fluorescence was observed with increasing concentrations of MIF-GN3, with maximal fluorescence observed at the highest concentration we investigated (1.0 μM) (FIG. 130). In contrast to DNP-GN3, a prozone effect was not observed with MIF-GN3-mediated MIF protein uptake at higher concentrations.

The ability of MIF-GN3 to mediate MIF endocytosis over a wide range of target protein concentrations was assessed. At a concentration of 100 nM MIF protein, several different concentrations of MIF-GN3 were found to mediate robust uptake of MIF-associated fluorophore (FIG. 131). A slight decrease in MIF uptake was observed when MIF-GN3 was present at a concentration 5.00 μM compared to lower concentrations. This decrease in endocytosis at high concentrations is consistent with the hook effect observed in ternary complex formation. An effective uptake of the MIF protein was also observed at a concentration of 10 nM: cells treated with 200 nM MIF-GN3 were found to be 4-fold more fluorescent than cells not treated with bifunctional molecule. In addition, at a concentration of 1 nM, some MIF-GN3 concentrations could mediate small increases in mean fluorescence intensity of the cell population. Based on these data, it was concluded that MIF-GN3 mediates the endocytosis of MIF protein across a wide range of target protein concentrations.

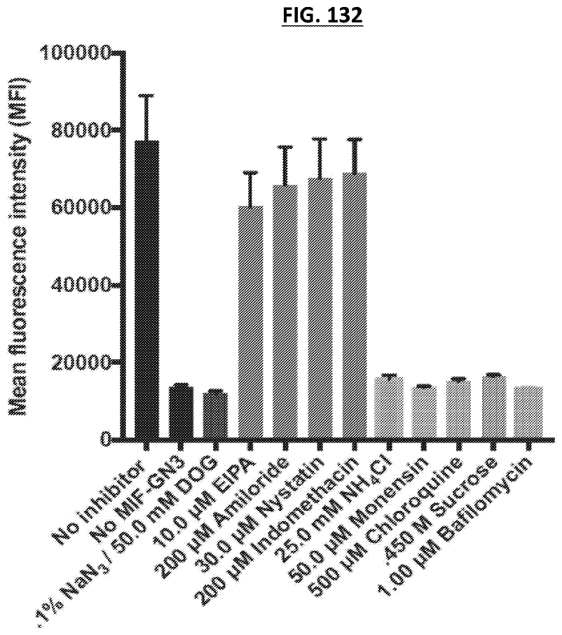

Next, inhibitors of various endocytic pathways were utilized to determine if MIF-GN3 mediates MIF endocytosis in a manner consistent with ASGPR (FIG. 132). When cells were incubated with the metabolic poisons sodium azide and 2-deoxyglucose, the mean fluorescence intensity of the cell population was decreased significantly (p=0.0001). This indicates that MIF-GN3 mediates the endocytosis of MIF protein in a manner that is dependent on ATP and cellular metabolism. The phagocytosis and macropinocytosis inhibitor amiloride did not impact cellular fluorescence significantly. In contrast, the closely related molecule EIPA significantly inhibited uptake (p=0.0199, *). A third inhibitor of these pathways, cytochalisin D, was toxic to cells under these conditions and was excluded from analysis. The inhibitors of caveolin-dependent endocytosis nystatin and indomethacin did not decrease the levels of intracellular fluorescence. A third inhibitor of caveolin-mediated endocytosis, genestein, was toxic to cells at the concentration tested. These toxicities were not observed in similar experiments which utilized α-DNP antibody as the target protein. One possibility for this enhanced toxicity is that HepG2 cells may have a cell surface protein which binds to MIF and sensitizes them to various inhibitors under stimulation by MIF oriteub. In the presence of clathrin-mediated endocytosis inhibitors ammonium chloride, monensin, chloroquine, sucrose, and bafilomycin, near background levels of intracellular fluorescence were observed (all conditions p=0.0001, ****).

### Example 38: Endocytosed MIF Protein is Trafficked to Late Endosomes

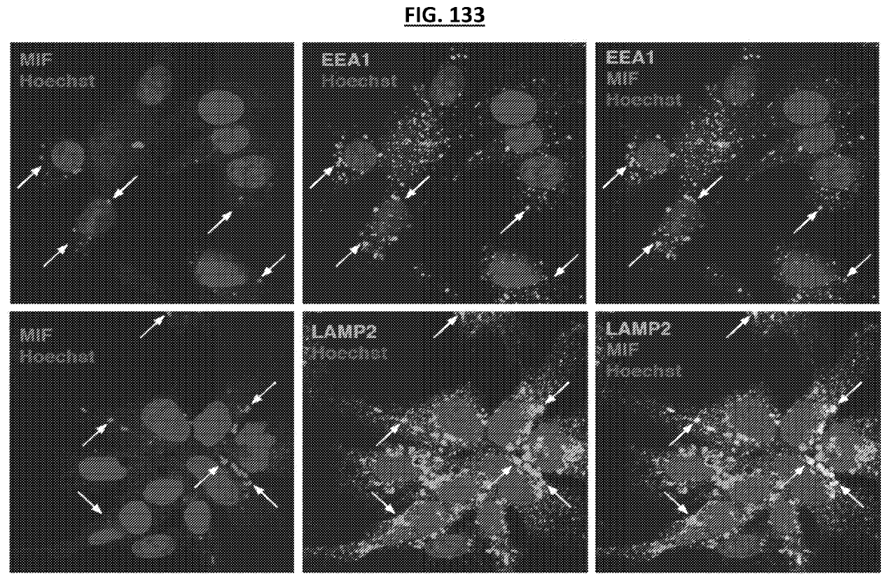

In order to investigate the subcellular localization of endocytosed MIF protein, colocalization studies were performed in HepG2 cells. After 12 hours of MIF-GN mediated endocytosis, several punctae displaying MIF-derived fluorescence were present in each cell. The location of these punctae did not overlap with the location of an antibody that detects the protein EEA1, which is present only in early endosomes (FIG. 133). In contrast, the studies show near-complete colocalization of MIF protein with the protein LAMP2, which is present in late endosomes and lysosomes. Based on these data, it was concluded that endocytosed MIF is trafficked to lysosomes where it may come into contact with lysosomal proteases.

### Example 39: MIF-GN3 Mediates the Depletion of Human MIF Protein from Serum in Mice

The ability of MIF-GN3 to mediate the depletion of injected human MIF from serum in mice was studied. First, the pharmacokinetics of the bifunctional molecule were investigated. Following a one mpk dose of MIF-GN3 in male nude mice, a half-life of 0.43 hours in serum was observed, with a maximum plasma concentration of 586.87 ng/mL after 15 minutes.

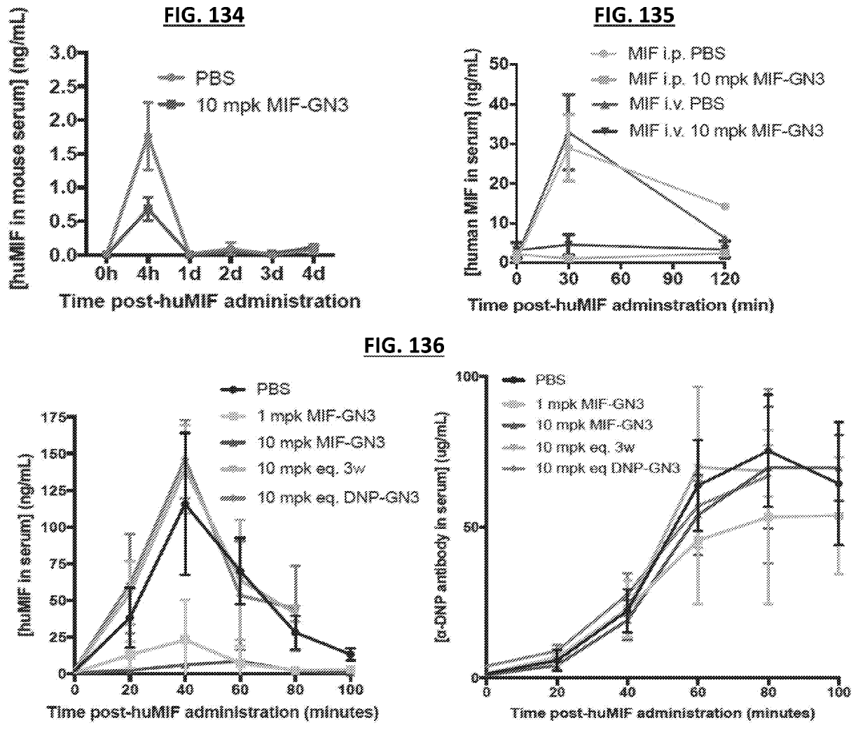

In vivo human MIF depletion experiments were undertaken to determine whether MIF-GN3 can mediate the depletion of injected recombinant human MIF from serum in mice. Mice were injected with five μg of the human MIF protein. In this experiment, one group of mice was also injected with a single 10 mpk dose of MLF-GN3 along with human MIF protein. It was observed that after four hours, the average level of human MIF in serum in the PBS-treated mice was 1.76 ng/mL (FIG. 134). In mice treated with 10 mpk MIF-GN3, however, the concentration of human MIF in serum was 0.68 ng/mL. One day after the initial injection, human MIF levels had reached background levels for both conditions. The difference between the serum levels of MIF in these two groups of mice was found to not be significant. It was hypothesized that, due to the short half-life of human MIF in serum, investigating human MIF levels at earlier time points may be more informative.

Next, an experiment was performed to determine whether MIF-GN3 could enhance MIF clearance at time points earlier than four hours. Mice were coinjected with recombinant human MIF and 10 mpk MIF-GN3 via either i.p. or i.v. routes. For the PBS control arms, human MIF was injected with PBS. It was observed that in the absence of MIF-GN3, there was a spike in huMIF levels in circulation after 30 minutes (FIG. 135). In the presence of MIF-GN3, however, levels of MIF protein remained low at 30 minutes and no spike in its concentration was observed. By two hours, the levels of human MIF in circulation decreased to background levels.

At 30 minutes, treatment with MIF-GN3 via both i.p. (p=0.0212, *) and i.v. (p=0.0455, *) gave significant depletion of human MIF protein from serum. In a further experiment, whether an inhibitor of the MIF protein (3w) is able to mediate the protein's depletion from serum was determined. A bifunctional molecule that mediates the depletion of α-DNP antibody from serum was also assayed as a negative control. Neither of these molecules was expected to mediate the formation of a ternary complex between human MIF protein and ASGPR. When human MIF was injected along with either 3w or DNP-GN3, no significant decreases in human MIF concentration in serum were observed compared to the PBS control (FIG. 136). In contrast, treatment with one mpk of MIF-GN3 led to a significant decrease in human MIF levels in serum at both the 40 (p=0.0001, ****) and 60 (p=0.0002, ***) minute time points. Treatment with ten mpk of MIF-GN3 also resulted in significant decreases in the concentration of human MIF in serum (40 minutes, p=0.0001, ****; 60 minutes, p=0.0005, ***).

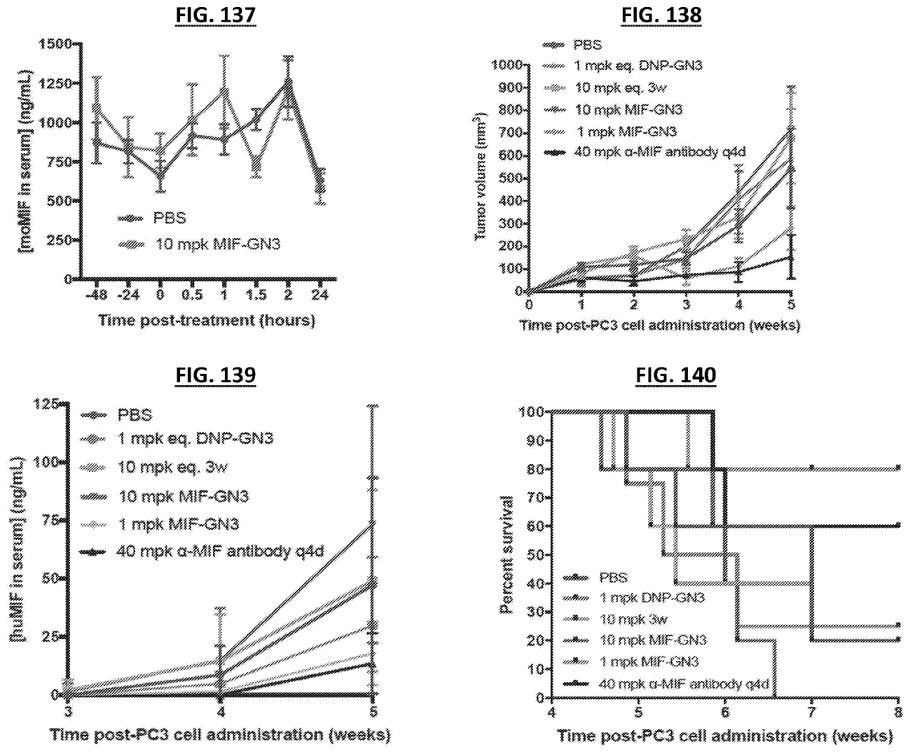

In order to confirm that mice injected with one or ten mpk MIF-GN3 had received the injection, and that the observed stability of MIF concentrations in MIF-GN3 treated mice was not the result of injection error, 200 μg of α-DNP antibody was coinjected with the MIF protein in this experiment. The levels of α-DNP antibody were not significantly changed by treatment with any bifunctional molecules (in all cases and time points, p>0.0702). Thus, the bifunctional molecule MIF-GN3 mediates MIF target protein depletion, while the control molecules 3w and DNP-GN3 do not. An investigation of the ability of MIF-GN3 to mediate the depletion of endogenous mouse MIF in vivo was next undertaken. Mice were treated with either PBS or 10 mpk of MIF-GN3. No significant decrease in mouse MIF concentrations was observed in the mice treated with MIF-GN3 (FIG. 137). One possible explanation for this finding is that MIF protein may be synthesized rapidly, and any increase in degradation mediated by MIF-GN3 may not be of a large enough magnitude to impact the protein's level in serum.

### Example 40: MIF-GN3 May Slow the Growth of Human Prostate Tumor PC3 Cells In Vivo

The ability of MIF-GN3 to mediate the depletion of MIF in a therapeutically relevant disease model was investigated. The human prostate cancer PC3 cell line has been demonstrated to grow more rapidly in the presence of human MIF homologs, as well as to be less proliferated in conditions in which MLF is depleted. An experiment was conducted to determine whether the bifunctional molecule MIF-GN3 can deplete sufficient human MIF from serum to slow PC3 tumor growth. Mice were injected with an aliquot of PC3 cells, and the size of the resultant tumors as well as the levels of human MIF in serum were monitored over five weeks. Tumor size arising from PC3 injection was observed to increase gradually over the course of the experiment. Five weeks after the initial PC3 cell injection, the first of the mice reached maximal tumor size (1000 mm²) and was sacrificed (FIG. 138). Treatment with PBS, DNP-GN3, the MIF inhibitor 3w, and 10 mpk MIF-GN3 was not found to decrease tumor growth in mice. Treatment of mice with MIF-GN3 at a dose of 1 mpk, however, resulted in a delay in tumor growth. Delayed tumor growth was also observed in mice treated with an α-MIF antibody, which was expected to neutralize the cytokine's pro-proliferation signaling. In addition to observing delayed tumor growth in mice treated with 1 mpk MIF-GN3 and the α-MIF antibody, decreased levels of human MIF protein was also observed in the serum of these mice (FIG. 139). The decrease in human MIF levels mediated by MIF-GN3 is consistent with a mechanism of action in which MIF-GN3 mediates the depletion and degradation of circulating MIF protein. While the α-MIF antibody would not necessarily be expected to decrease the levels of human MIF, it is possible that the antibody mediates MIF neutralization in a manner which masks its signal as determined by ELISA. Alternatively, the α-MIF antibody may mediate depletion or degradation of huMIF by some endocytic mechanism. Another possible explanation is that the α-MIF antibody decrease serum levels of active human MIF to such an extent at early time points that PC3 cells proliferate much more slowly, and therefore fewer cells are actively secreting human MIF.

Mice were sacrificed when their tumor volumes reached 1000 mm³. Mice treated with 1 mpk MIF-GN3 showed 80% survival eight weeks after the initial PC3 cell injection, compared to 60% survival in the α-MIF antibody treated arm (FIG. 140). At this same time point, less than 25% of the mice in any other arm had survived. Based on these data, it was concluded that MIF-GN3 at a dose of 1 mpk is able to mediate depletion of huMIF from serum in mice, and that depletion of huMIF slows PC3 tumor cell growth in vivo.

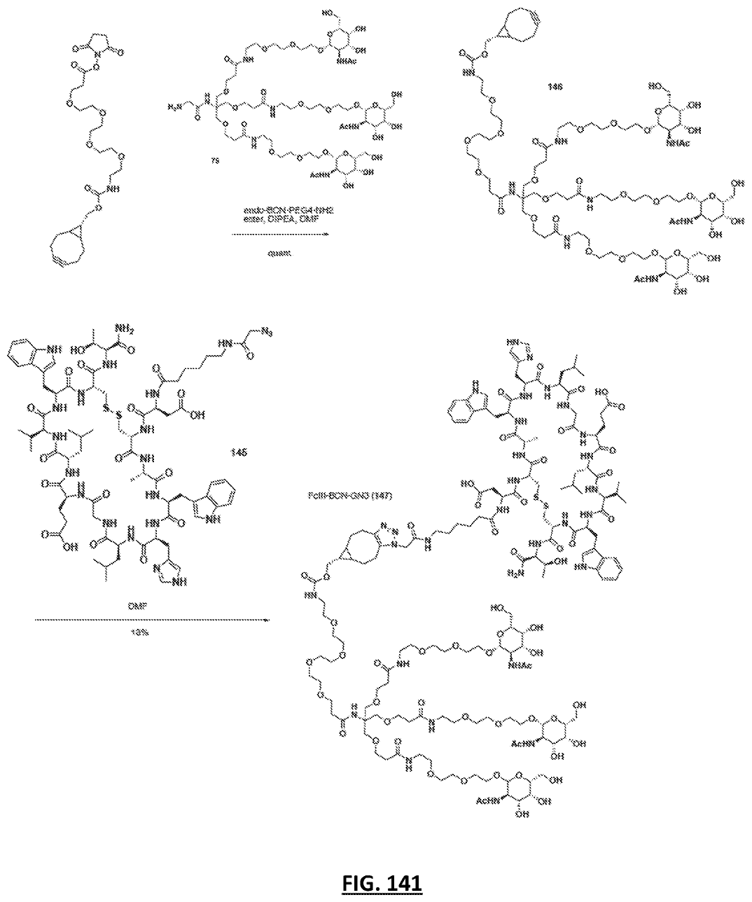

### Example 41: Synthesis of Bifunctional Molecule FcIII-GN3 (FIG. 141)

The bifunctional molecule FcIII-BCN-GN3 was synthesized in much the same manner as FcIII-GN3 (FIG. 78). Solid phase peptide synthesis was carried out to synthesize the azide-terminated FcIII peptide 145. Separately, the bicyclononye (BCN) alkyne terminated tri-GalNAc motif was generated by reacting amine 75 with a commercially available NHS BCN molecule. The crude product was used without purification. Mixing compound 145 and 146 together in dimethylformamide generated the final compound FcIII-BCN-GN3 (147).

### Example 42: FcIII-GN3 Mediates the Endocytosis and Lysosomal Trafficking of Human IgG

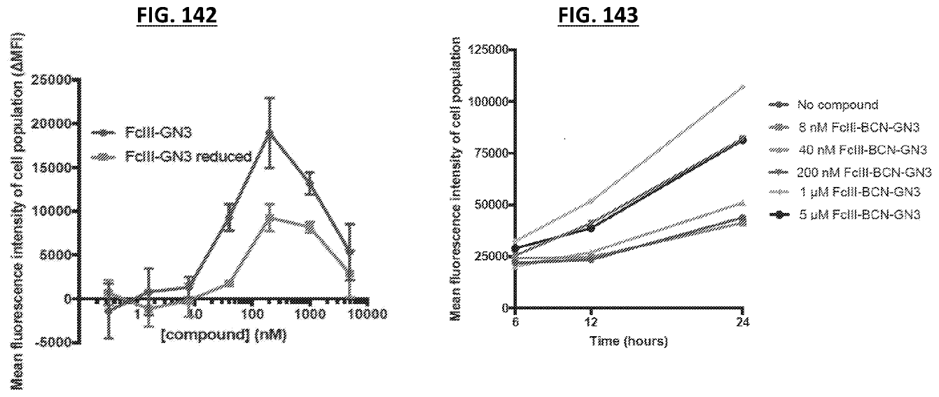

The ability of the bifunctional molecule FcIII-GN3 to mediate IgG uptake by HepG2 cells was investigated. Cells were incubated with Alexa 488-labeled human IgG and treated with varying levels of FcIII-GN3. Two forms of FcIII-GN3 were investigated: both the reduced linear form and the oxidized cyclized form. The reduced form of FcIII has been previously reported to not bind strongly to human IgG (R. L. Dias et al., Journal of the American Chemical Society, 2006, 128:2726-2732). It was observed that antibody endocytosis was dependent on the concentration of FcIII-GN3, with maximal IgG uptake observed at a concentration of 200 nM (FIG. 142). In addition, a hook effect consistent with ternary complex formation was observed. The reduced form of FcIII-GN3 was also found to induce human IgG endocytosis, albeit to a lesser extent than the cyclized form. One possible explanation for this observation is that over the time scale of this experiment (six hours), oxidation of the disulfide is carried out in cell culture via air oxidation. A small amount of the linear FcIII-GN3 may therefore be oxidized to the cyclized form. This reduced compound may be the agent responsible for mediating human IgG endocytosis under these conditions. Due to possible synthetic challenges in the production of FcIII-GN3, the biological activity of FcIII-BCN-GN3, which differs from FcIII-GN3 only in the structure of the linking triazole group, was also explored. FcIII-BCN-GN3 proved effective at mediating IgG endocytosis across a range of concentrations and times (FIG. 143). 1 μM FcIII-BCN-GN3 was the most effective concentration, followed by concentrations of 5 μM and 200 nM. These dosing trends are consistent with the concentration-dependent hook effect observed in systems in which a ternary complex is formed. Based on these in vitro data, FcIII-GN3 or FcIII-BCN-GN3 are viable for further study.

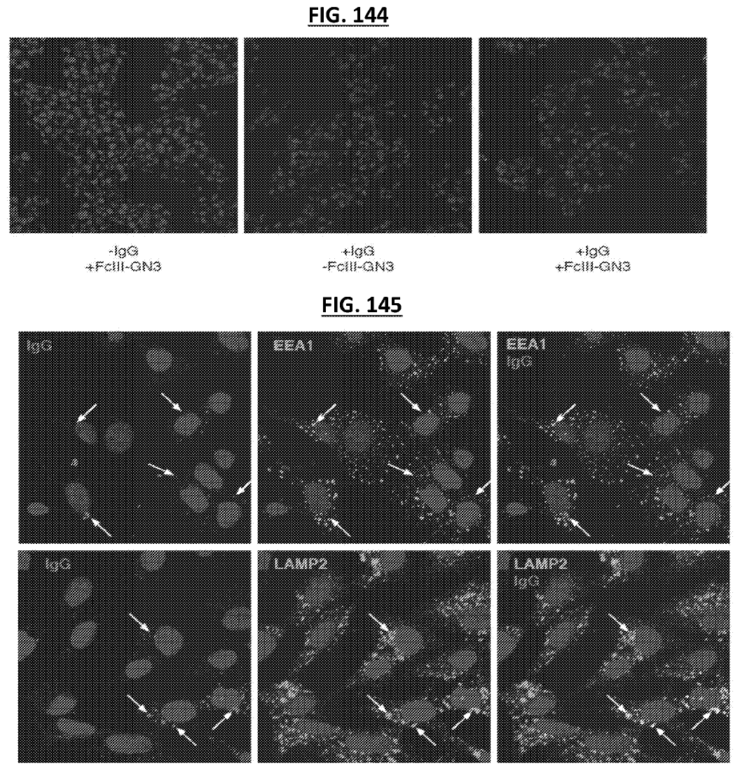

In order to investigate the subcellular localization of endocytosed human IgG, fluorescence colocalization studies were performed. Cells were incubated with both fluorescently labeled human IgG and FcIII-GN3. In the absence of human IgG, no background fluorescence arising in the Alexa 568 channel was observed (FIG. 144). In the absence of FcIII-GN3 but presence of fluorescently labeled human IgG several fluorescent punctae were observed in cells, possibly due to non-specific antibody uptake. One possible explanation for this observation is that at the concentration of human IgG tested (100 nM), FcRN on the surface of HepG2 cells may mediate the endocytosis of a low level of human IgG (R. J. Ober et al., International immunology, 2001, 13:1551-1559). In cells treated with both human IgG and FcIII-GN3, an increase of fluorescent punctae in cells was observed, resulting from IgG endocytosis. Colocalization experiments were performed to determine whether endocytosed IgG is trafficked to early or late endosomes. No overlap with the protein EEA1, which is a marker of early endosomes, was observed (FIG. 145). In contrast, strong colocalization was observed with the late endosome and lysosome protein LAMP2. Based on these data, it was concluded that FcIII-GN3 mediates the lysosomal trafficking of endocytosed IgG.

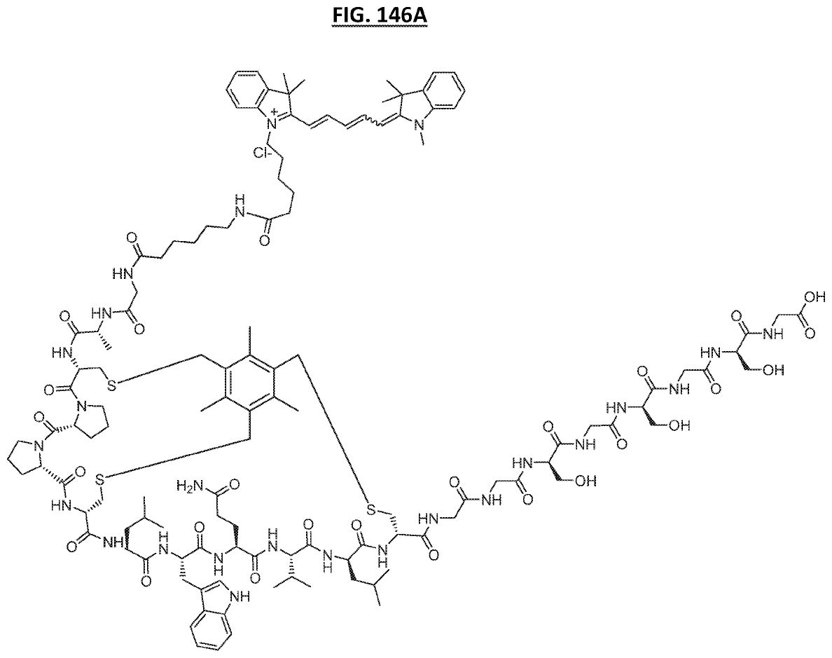

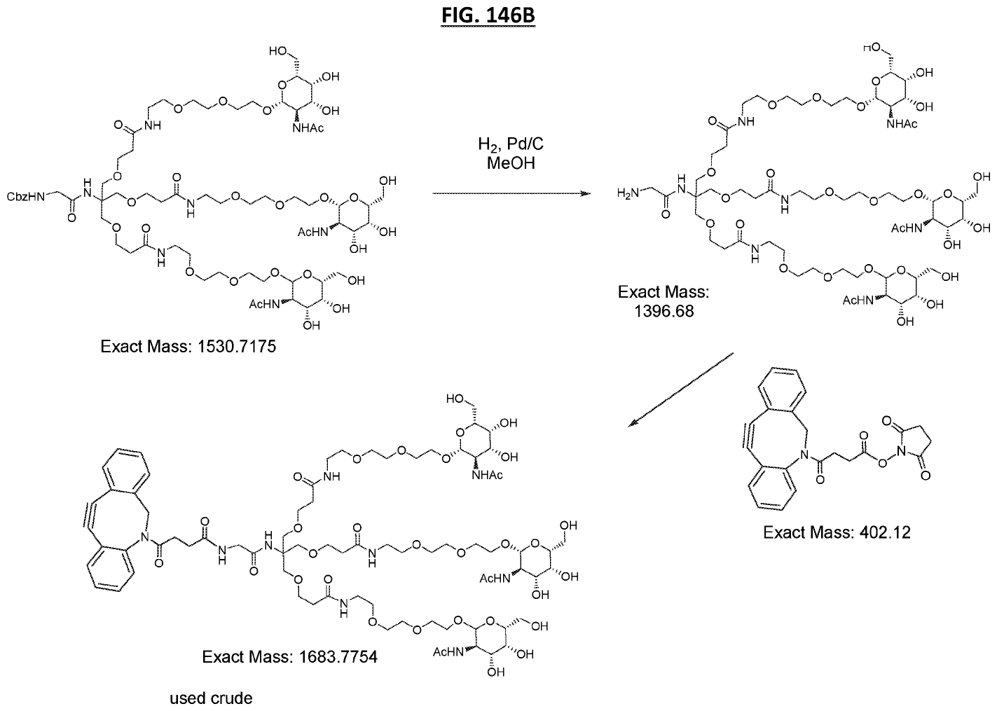

### Example 43: TNF Binder and Synthesis of -[CON]ₕ-[Linker]ᵢ-[CON]_{h′}-[CRBM]_{j′} Fragment (FIGS. 146a-146b)

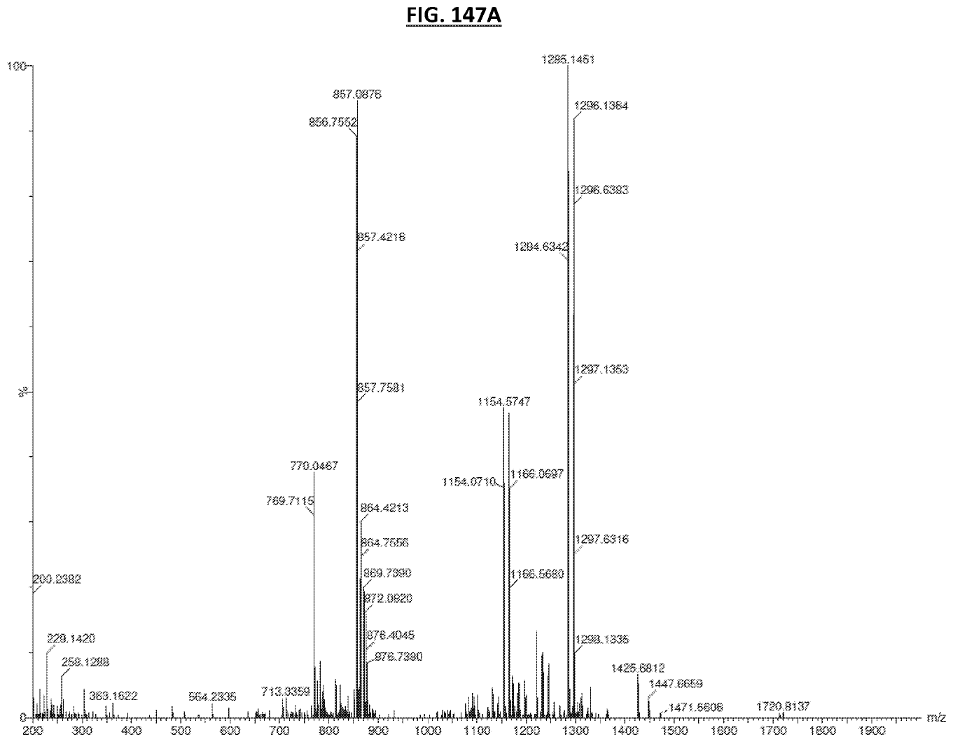

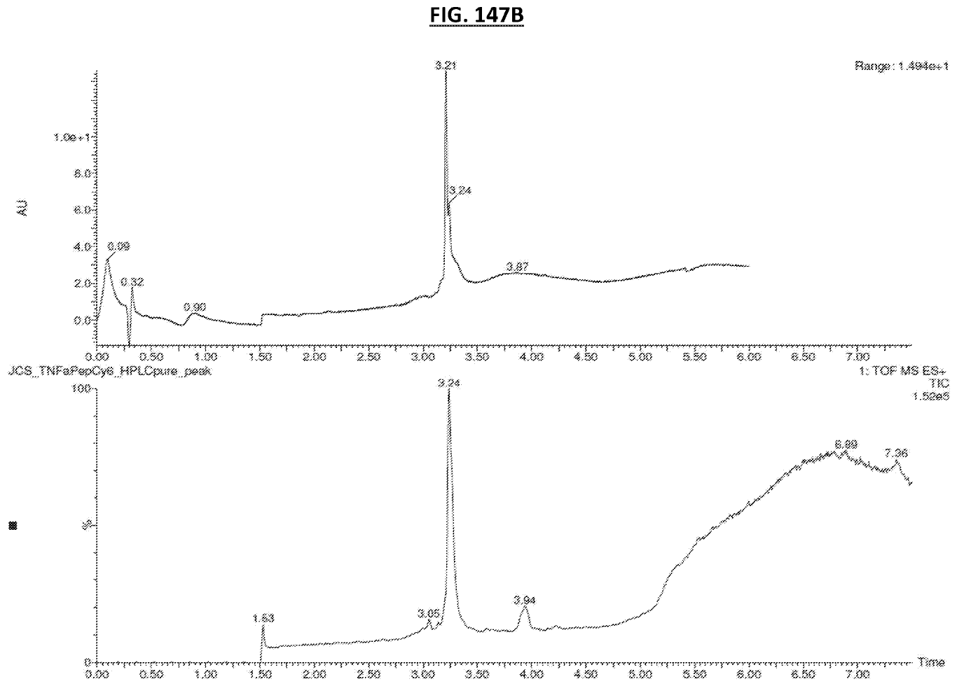

Synthesis of the TNF binder (FIG. 146A): The crude peptides (1 mM) in 1 ml 70% (v/v) 20 mM NH4HCO₃ pH 8 and 30% (v/v) ACN were reacted with TBMB (1.2 mM) for 1 h at room temperature. The reaction product was purified by reversed-phase HPLC using a C18 column and gradient elution with a mobile phase composed of ACN and 0.1% (v/v) aqueous trifluoroacetic acid (TFA) solution at a flow rate of 2 ml min⁻¹. The purified peptides were freeze-dried and dissolved in DMSO or a buffer of 50 mM Tris-Cl pH 7.8, 150 mM NaCl for measurement. The K_{d} of the TNFα dimer is reported to be 5.2 nM (Luzi, S. et al., Protein Engineering, Design & Selection, 2015, 28:45-52). FIGS. 147A-147B provide characterization of the TNF binder.

FIG. 146B depicts the synthesis of the -[CON]ₕ-[Linker]ᵢ-[CON]_{h′}-[CRBM]_{j′} fragment of formula (II). This fragment can be coupled to the TNF binder through reaction of the alkyne of -[CON]ₕ-[Linker]ᵢ-[CON]_{h′}-[CRBM]_{j′} fragment with the indole ring on the TNF binder.

### Enumerated Embodiments

The following enumerated embodiments are provided, the numbering of which is not to be construed as designating levels of importance.

Embodiment 1 provides a compound comprising formula (I), or a salt, geometric isomer, stereoisomer, or solvate thereof:

[Protein binder]_{k′}-[CON]ₕ-[Linker]ᵢ-[CON]_{h′}-[CRBM]_{j′} (I),

wherein: the Protein binder is a molecule that binds to an extracellular protein;
the CRBM is a cellular receptor binding moiety that binds to at least one receptor on the surface of a degrading cell in a subject, whereby binding of (I) leads to endocytosis and degradation of the extracellular protein; each CON is independently a bond or a group that covalently links a Protein binder to an CRBM, a Protein binder to a Linker, and/or a Linker to a CRBM; the Linker is a group having a valence ranging from 1 to 15; k′ is an integer ranging from 1 to 15; h is an integer ranging from 0 to 15; i is an integer ranging from 0 to 15; h′ is an integer ranging from 0 to 15; j is an integer ranging from 1 to 15.

Embodiment 2 provides a compound comprising formula (II), or a salt, geometric isomer, stereoisomer, or solvate thereof:

[TNF binder]_{k′}-[CON]ₕ-[Linker]ᵢ-[CON]_{h′}-[CRBM]_{j′} (II),

wherein: the TNF binder is a molecule that binds to TNF; the CRBM is a cellular receptor binding moiety that binds to at least one receptor on the surface of a degrading cell in a subject, whereby binding of (II) leads to endocytosis and degradation of TNF; each CON is independently a bond or a group that covalently links a TNF binder to an CRBM, a TNF binder to a Linker, and/or a Linker to a CRBM; the Linker is a group having a valence ranging from 1 to 15; k′ is an integer ranging from 1 to 15; h is an integer ranging from 0 to 15; i is an integer ranging from 0 to 15; h′ is an integer ranging from 0 to 15; j is an integer ranging from 1 to 15.

Embodiment 3 provides a compound comprising formula (III), or a salt, geometric isomer, stereoisomer, or solvate thereof:

[AATM]_{k′}-[CON]ₕ-[Linker]ᵢ-[CON]_{h′}-[CRBM]_{j′} (III),

wherein: the AATM is a molecule that binds to an autoantibody; the CRBM is a cellular receptor binding moiety that binds to at least one receptor on the surface of a degrading cell in a subject, whereby binding of (III) leads to endocytosis and degradation of the autoantibody; each CON is independently a bond or a group that covalently links an AATM to an CRBM, an AATM to a Linker, and/or a Linker to a CRBM; the Linker is a group having a valence ranging from 1 to 15; k′ is an integer ranging from 1 to 15; h is an integer ranging from 0 to 15; i is an integer ranging from 0 to 15; h′ is an integer ranging from 0 to 15; j is an integer ranging from 1 to 15.

Embodiment 4 provides the compound of any one of Embodiments 1-3, wherein the valence of the Linker is 1, 2, or 3.

Embodiment 5 provides the compound of any one f Embodiments 1-4, wherein k′ is 1, 2, or 3.

Embodiment 6 provides the compound of any one of Embodiments 1-5, wherein j is 1, 2, or 3.

Embodiment 7 provides the compound of any one of Embodiments 1-6, wherein h is 1, 2, or 3.

Embodiment 8 provides the compound of any one of Embodiments 1-7, wherein h′ is 1, 2, or 3.

Embodiment 9 provides the compound of any one of Embodiments 1-8, wherein i is 1, 2, or 3.

Embodiment 10 provides the compound of any one of Embodiments 1-9, wherein at least one of h, h′, and i is at least 1.

Embodiment 11 provides the compound of any one of Embodiments 1-10, wherein k′, j′, h, h′, and i are each independently 1, 2, or 3.

Embodiment 12 provides the compound of any one of Embodiments 1-11, wherein k′ is 1, and j′ is 1, 2, or 3.

Embodiment 13 provides the compound of any one of Embodiments 1 or 4-12, which is:

[Protein binder]-[CON]₀₋₁-[Linker]-[CON]₀₋₁-[CRBM] (Ia).

Embodiment 14 provides the compound of any one of Embodiments 2 or 4-12, which is:

[TNF binder]-[CON]₀₋₁-[Linker]-[CON]₀₋₁-[CRBM] (IIa).

Embodiment 15 provides the compound of any one of Embodiments 3-12, which is:

[AATM]-[CON]₀₋₁-[Linker]-[CON]₀₋₁-[CRBM] (IIIa).

Embodiment 16 provides the compound of any one of Embodiments 1-15, wherein the degrading cell comprises a hepatocyte.

Embodiment 17 provides the compound of any one of Embodiments 1-16, wherein the CRBM is a folic acid (folate) receptor binder, mannose receptor binder, mannose-6-phosphate (M6P) receptor binder, low density lipoprotein receptor-related protein 1 (LRP1) receptor binder, low density lipoprotein receptor (LDLR) binder, FcγRI receptor binder, transferrin receptor binder, macrophage scavenger receptor binder, G-Protein coupled receptor binder, or asialoglycoprotein receptor (ASGPR) binder.

Embodiment 18 provides the compound of any one of Embodiments 1-17, wherein the CRBM is:
(a) a folic acid (folate) receptor binder comprising at least one of folic acid, methotrexate, premetrexed, or a biologically active fragment thereof;
(b) a mannose receptor binder comprising at least one of:

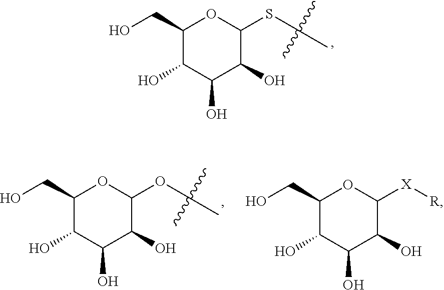

wherein: X is S or O, R is selected from the group consisting of:

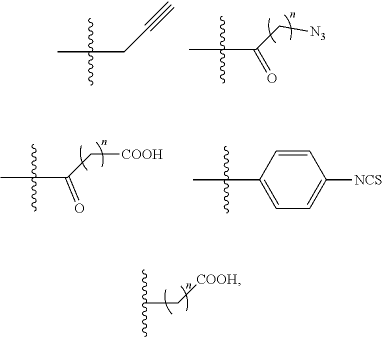

and each occurrence of ‘n’ is independently 1, 2, 3, 4, 5, 6, 7, 8, 9, 10, 11, 12, 13, 14, 15, 16, 17, 18, 19, or 20; and
a polymeric molecule selected from the group consisting of:

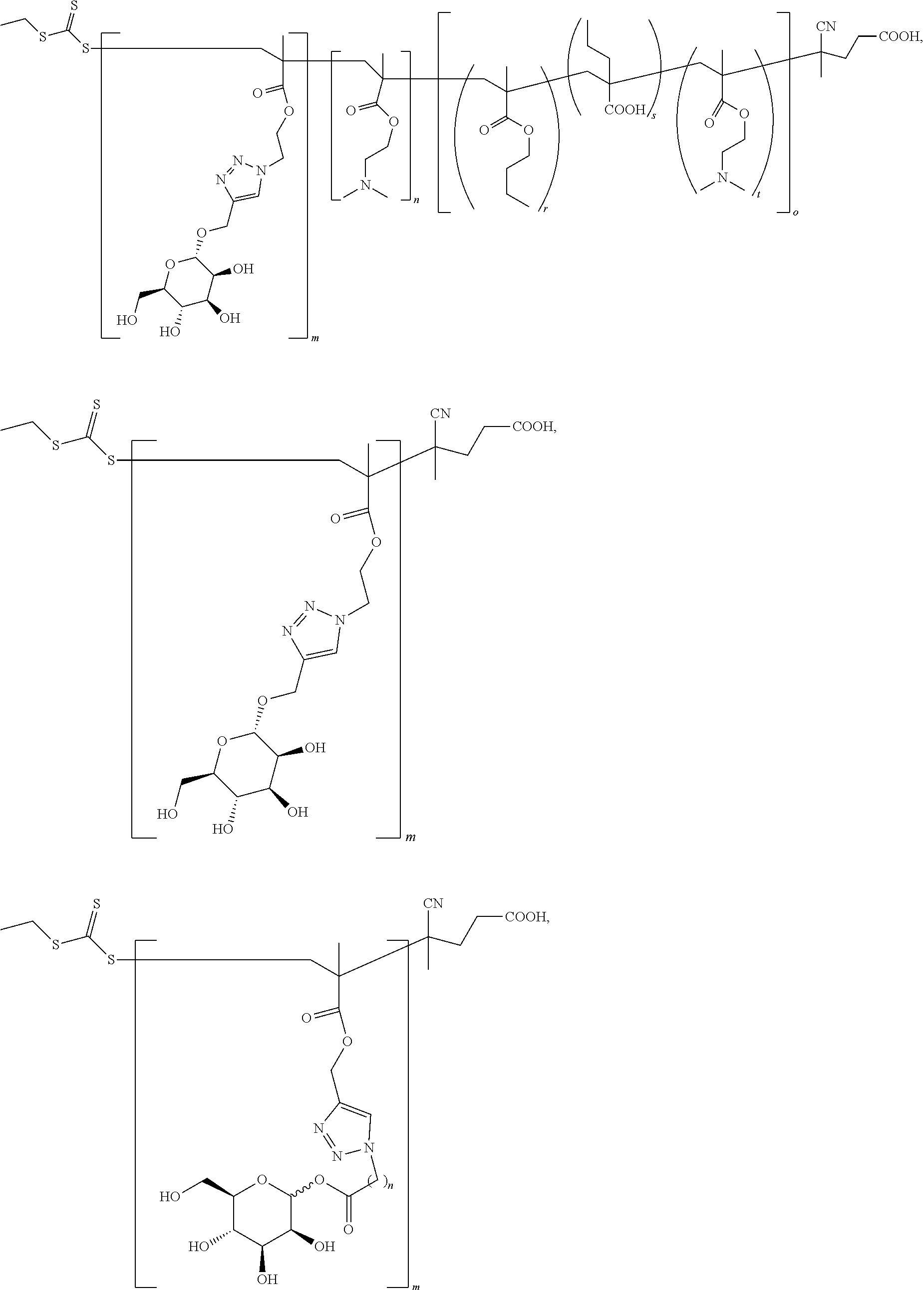

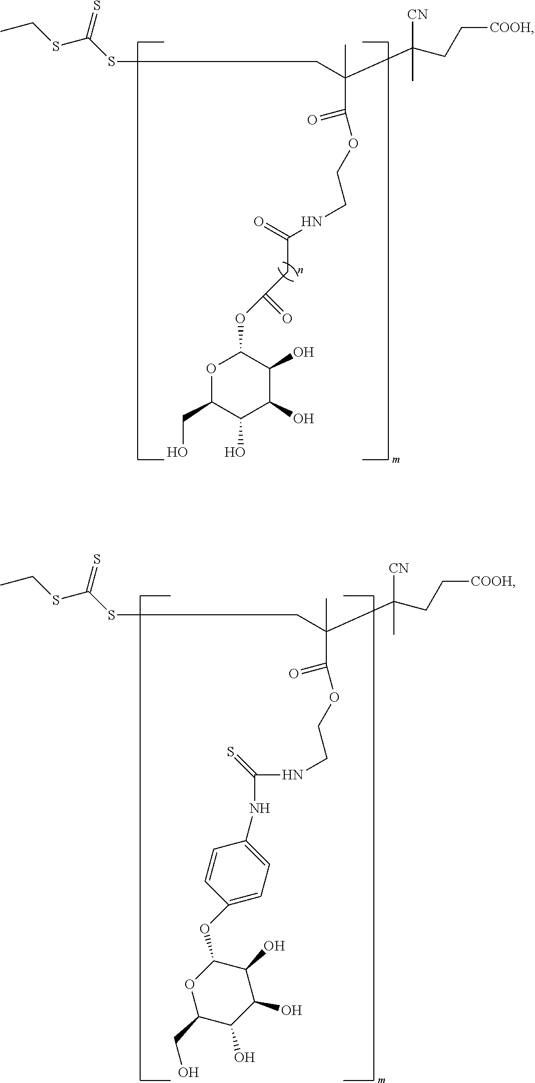

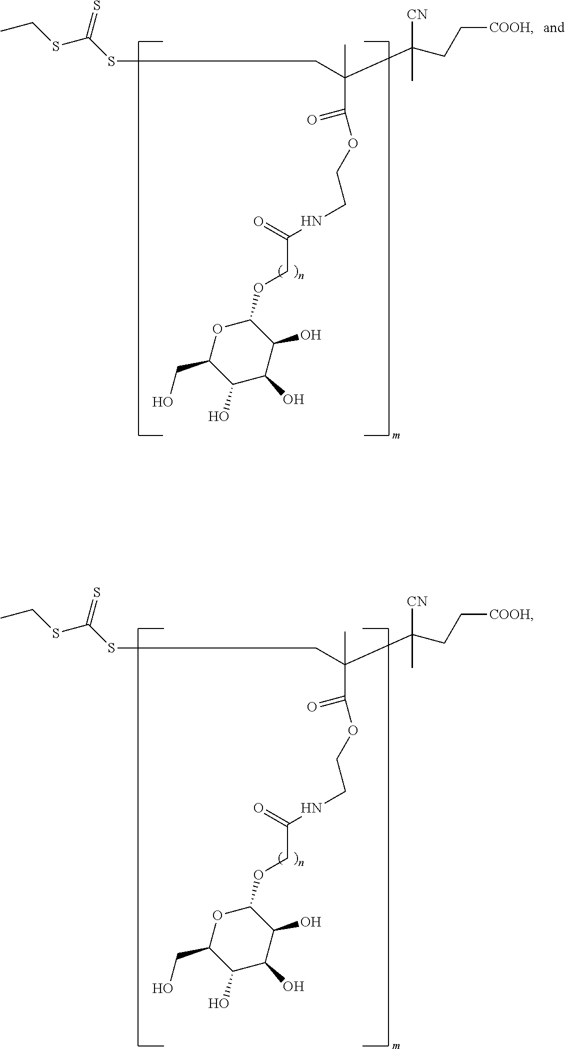

m is an integer from 1 to 100,
r, s, t, and o are each independently an integer from 0 to 100, and the COOH in the
polymeric molecule is derivatized with the Protein binder, the TNF binder, or the AATM;
(c) a mannose-6-phosphate (M6P) receptor binder comprising at least one of.

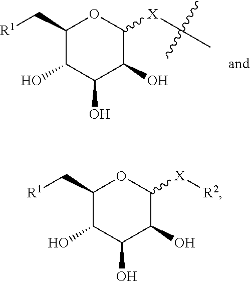

wherein X is O or S, R¹ is selected from the group consisting of

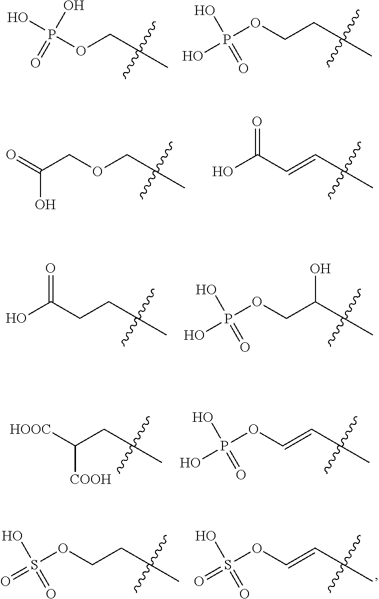

R² is selected from the group consisting of:

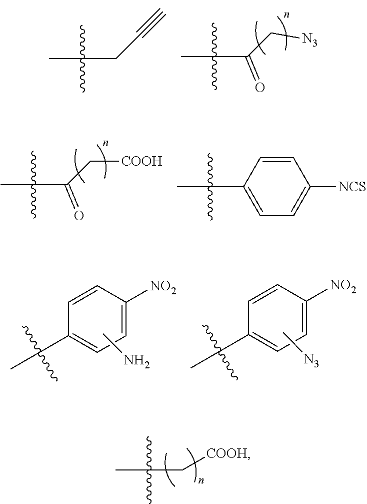

and each occurrence of ‘n’ is independently 1, 2, 3, 4, 5, 6, 7, 8, 9, 10, 11, 12, 13, 14, 15, 16, 17, 18, 19, or 20;
   a polymeric molecule

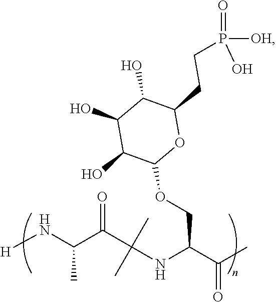

wherein n is an integer from 1 to 100;
a compound selected from:

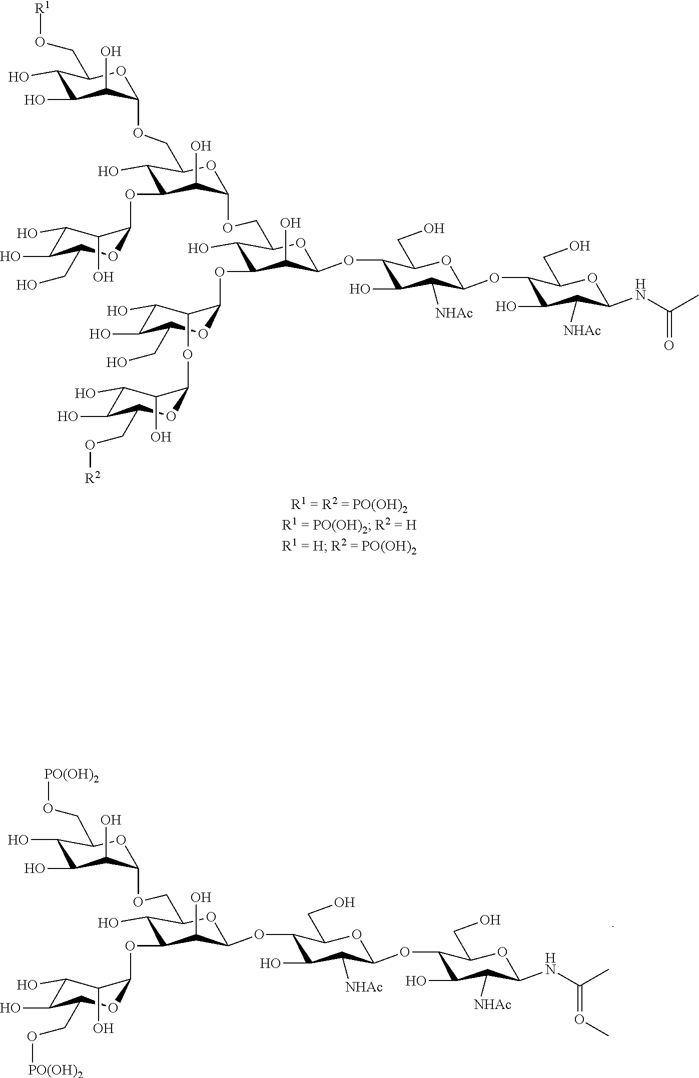

a compound selected from:

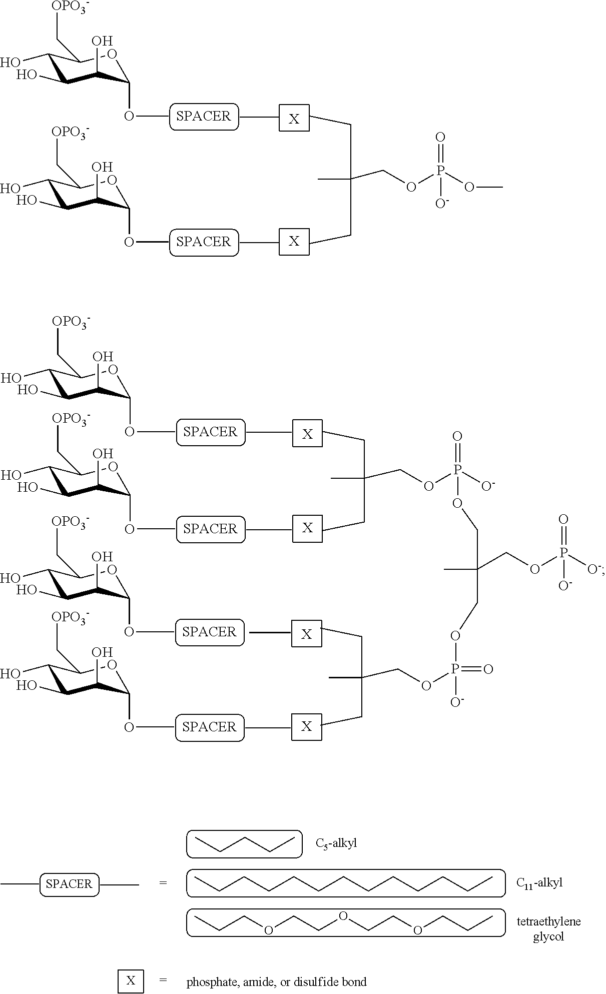

(d) a low density lipoprotein receptor-related protein 1 (LRP1) receptor binder comprising at least one amino acid sequence of SEQ ID NOs:1-9;
(e) a low density lipoprotein receptor (LDLR) binder comprising at least one amino acid sequence of SEQ ID NOs:10-35;
(f) a FcγRI receptor binder comprising at least one amino acid sequence of SEQ ID NOs:36-52;
(g) a transferrin receptor binder comprising at least one amino acid sequence of SEQ ID NOs:53-59 and 74;
(h) a macrophage scavenger receptor binder comprising at least one amino acid sequence of SEQ ID NOs:60-65;
(i) a G-protein coupled receptor binder comprising at least one of:

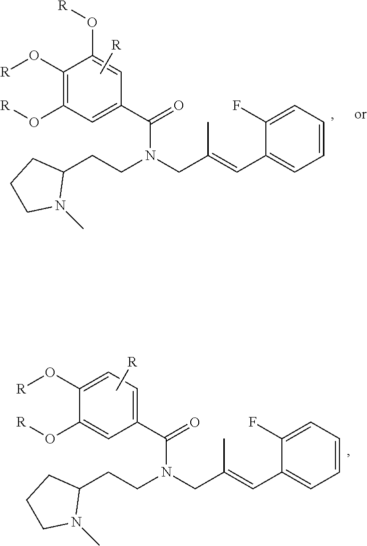

wherein each occurrence of R is independently H or C₁-C₆ alkyl;
(j) an asialoglycoprotein receptor (ASGPR) binder comprising:

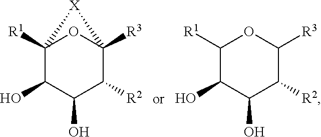

wherein:
X is a linker of 1-4 atoms in length and comprises O, S, N(R^{N1}), or C(R^{N1})(R^{N1}) groups, such that:
   when X is a linker of 1 atom in length, X is O, S, N(R^{N1}), or C(R^{N1})(R^{N1}),
   when X is a linker of 2 atoms in length, no more than 1 atom of X is O, S, or N(R^{N1}),
   when X is a linker of 3 or 4 atoms in length, no more than 2 atoms of X are independently O, S, or N(R^{N1});
   wherein each occurrence of R^{N1} is independently H or C₁-C₃ alkyl optionally substituted with 1-3 independently selected halogens and/or 1-2 hydroxyl groups;
R¹ and R³ are each independently H, —(CH₂)_{K}OH, —(CH₂)_{K}C(C₁-C₄ alkyl) optionally substituted with 1-3 independently selected halogens, C₁-C₄ alkyl optionally substituted with 1-3 independently selected halogens, —(CH₂)_{K}(vinyl), —O(CH₂)_{K}(vinyl), —(CH₂)k(alkynyl), —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens; or
R¹ and R³ are each independently Ph(CH₂)_{K}—, which is optionally substituted with: 1-3 independently selected halogens; C₁-C₄ alkyl optionally substituted with 1-3 independently selected halogens and/or 1-2 hydroxyl groups; or C₁-C₄ alkoxy optionally substituted with 1-3 independently selected halogens and/or 1-2 hydroxyl groups; or
R¹ and R³ are each independently a group of structure:

—O—(CH₂)_{K′}—CH(OH)—(CH₂)_{K′}-R⁷,
wherein:
   R⁷ is: C₁-C₄ alkoxy optionally substituted with 1-3 independently selected halogens and/or 1-2 hydroxy groups; —NR^{N3}R^{N4}; or —(CH₂)_{K′}—O—(CH₂)_{K}—CH₂—CH═CH₂;
   K is 0, 1, 2, 3, or 4;
   K′ is 1, 2, 3, or 4;
   each occurrence of R^{N3} is independently H or C₁-C₃ alkyl optionally substituted with 1-3 independently selected halogens and/or 1-2 hydroxyl groups;
   each occurrence of R^{N4} is independently H, C₁-C₃ alkyl optionally substituted with 1-3 independently selected halogens and/or 1-2 hydroxyl groups, or Ph-(CH₂)x-; or
R¹ and R³ are each independently selected from the group consisting of:
   (CH₂)_{K}OH,

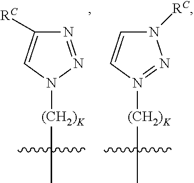

L¹-═-, L¹-(CH₂)_{K}—, and CYC-(CH₂)_{K}—,
wherein CYC is selected from the group consisting of:

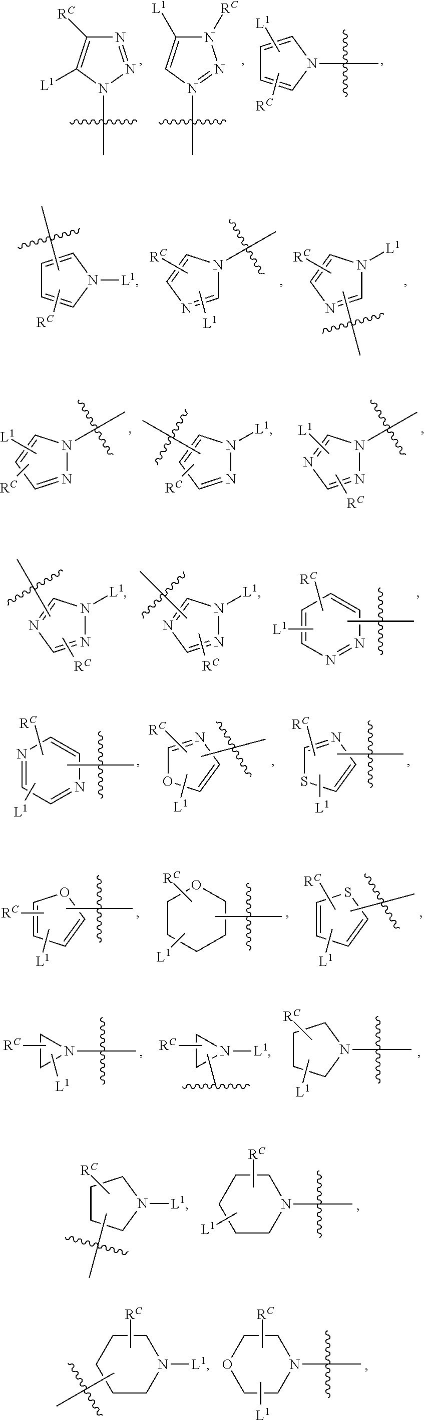

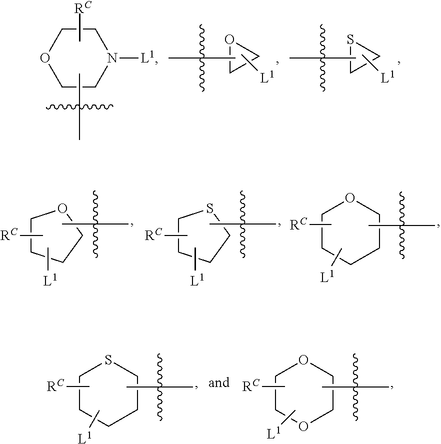

wherein:
   the bond marked with [symbol] indicating the site on CYC whereto —(CH₂)_{K} is connected;
   L¹ is a bond, -Linker, —CON-Linker, or —CON-Linker-CON,
   R^{C} is absent, H, C₁-C₄ alkyl optionally substituted with 1-3 optionally substituted halogens and/or 1-2 hydroxyl groups, or a group of structure:

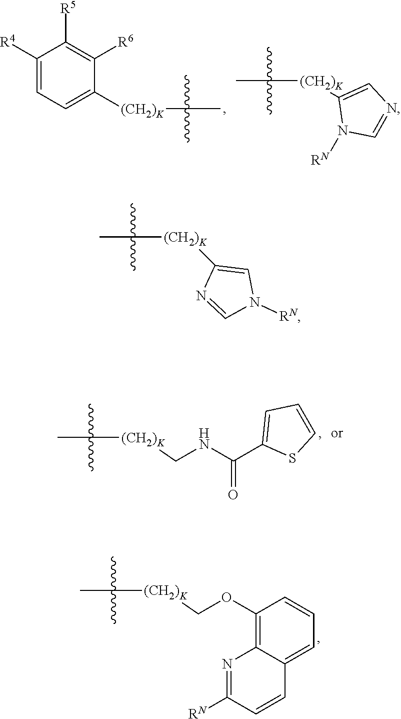

wherein:
R⁴, R⁵, and R⁶ are each independently H, F, Cl, Br, I, CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens, C₁-C₃-alkoxy optionally substituted with 1-3 independently selected halogens, —(CH₂)_{K}COOH, —(CH₂)_{K}C(═O)O—(C₁-C₄ alkyl) optionally substituted with 1-3 independently selected halogens, O—C(═O)—(C₁-C₄ alkyl) optionally substituted with 1-3 independently selected halogens, or —C(═O)—(C₁-C₄ alkyl) optionally substituted with 1-3 independently selected halogens;
each occurrence of R^{N} is independently H or C₁-C₃ alkyl optionally substituted with 1-3 independently selected halogens and/or 1-2 hydroxyl groups;
each occurrence of R^{N2} is independently H or C₁-C₃ alkyl optionally substituted with 1-3 independently selected halogens and/or 1-2 hydroxyl groups;
or
R¹ and R³ are each independently (C₃-C₈ saturated carbocyclic)-(CH₂)_{K}—, wherein the carbocyclic is further substituted with -L¹ and —Re;
R² is —(CH₂)_{K}—N(R^{N1})—C(═O)R^{AM}, wherein:
   R^{AM} is H, C₁-C₄ alkyl optionally substituted with 1-3 independently selected halogens and/or 1-2 hydroxyl groups, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, —C(═O)(C₁-C₄ alkyl) optionally substituted with 1-3 independently selected halogens, or —(CH₂)_{K}—NR^{N3}R^{N4}; or
R² is

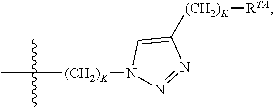

wherein:
   R^{TA} is H, CN, NR^{N1}R^{N2}, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens, or
   R^{TA} is C₃-C₁₀ aryl or a 3- to 10-membered heteroaryl group containing 1-5 non-carbon ring atoms, each of the aryl or heteroaryl groups being optionally substituted with 1-3 groups independently selected from CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens and/or 1-2 hydroxyl groups, -(C₁-C₃-alkoxy) optionally substituted from 1-3 independently selected halogens, —(CH₂)_{K}COOH, —(CH₂)_{K}C(═O)O—(C₁-C₄ alkyl) optionally substituted with 1-3 independently selected halogens, —OC(═O)(C₁-C₄ alkyl) optionally substituted with 1-3 independently selected halogens, or —(CH₂)_{K}C(═O)—(C₁-C₄ alkyl) optionally substituted with 1-3 independently selected halogens, or
   R^{TA} is

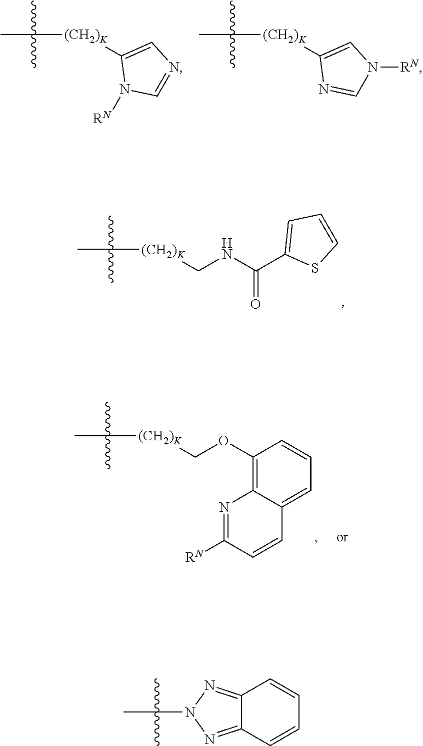

optionally substituted with 1-3 C₁-C₃ alkyl groups optionally substituted with 1-3 independently selected halogens, or
R^{TA} is

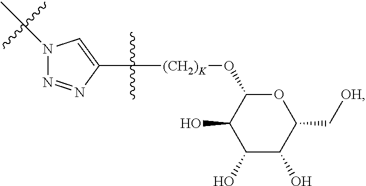

wherein each —(CH₂)_{K} group is optionally substituted with 1-4 C₁-C₃ alkyl groups optionally substituted with 1-3 fluoro groups or 1-2 hydroxyl groups.

Embodiment 19 provides the compound of Embodiment 18, wherein:
the X in ASGPRBM is —O—C(R^{N1})(R^{N1})—, —C(R^{N1})(R^{N1})—O—, —S—C(R^{N1})(R^{N1})—, —C(R^{N1})(R^{N1})—S—, —N(R^{N1})—C(R^{N1})(R^{N1})—, —C(R^{N1})(R^{N1})—N(R^{N1})—, or —C(R^{N1})(R^{N1})—C(R^{N1})(R^{N1})—, when X is 2 atoms in length;
the X in ASGPRBM is —O—C(R^{N1})(R^{N1})—C(R^{N1})(R^{N1})—, —C(R^{N1})R^{N1})—O—C(R^{N1})(R^{N1})—, —O—C(R^{N1})(R^{N1})—O—, —O—C(R^{N1})(R^{N1})—S—, —O—C(R^{N1})(R^{N1})—N(R^{N1})—, —S—C(R^{N1})(R^{N1})—C(R^{N1})(R^{N1})—, —C(R^{N1})(R^{N1})—S—C(R^{N1})(R^{N1})—, —C(R^{N1})(R^{N1})—C(R^{N1})(R^{N1})—S, —S—C(R^{N1})(R^{N1})—S—, —S—C(R^{N1})(R^{N1}) O—, —S—C(R^{N1})(R^{N1})—N(R^{N1})—, —N(R^{N1})—C(R^{N1})(R^{N1})—C(R^{N1})(R^{N1}), —C(R^{N1})(R^{N1})—N(R^{N1})—C(R^{N1})(R^{N1})—, —C(R^{N1})(R^{N1})—C(R^{N1})(R^{N1})—N(R^{N1})—, —N(R^{N1})—C(R^{N1})(R^{N1})—N(R^{N1})—, or —C(R^{N1})(R^{N1})—C(R^{N1})(R^{N1})—C(R^{N})(R^{N1}), when X is 3 atoms in length; or
the X in ASGPRBM is —O—C(R^{N1})(R^{N1})—C(R^{N1})(R^{N1})—C(R^{N1})(R^{N1}), —C(R^{N1})(R^{N1})O—C(R^{N1})(R^{N1})—C(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})-, —C(R^{N1})(R^{N1})—S—C(R^{N1})(R^{N1})—C(R^{N1})(R^{N1})—, —C(R^{N1})(R^{N1})—C(R^{N1})(R^{N1})—S—C(R^{N1})(R^{N1})—, —S—C(R^{N1})(R^{N1})—S—C(R^{N1})(R^{N1})—, —N(R^{N1})—C(R^{N1})(R^{N1})(R^{N1})(R^{N1})—C(R^{N1})(R^{N1})—, or —C(R^{N1})(R^{N1})—N(R^{N1})—C(R^{N1})(R^{N1})—C(R^{N1})(R^{N1})—, when X is 4 atoms in length.

Embodiment 20 provides the compound of any one of Embodiments 18-19, wherein X is OCH₂ and R^{N1} is H, or wherein X is CH₂O and R^{N1} is H.

Embodiment 21 provides the compound of any one of Embodiments 18-20, wherein the ASGPRBM comprises the structure:

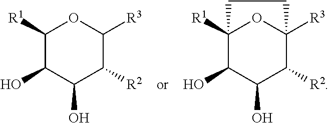

Embodiment 22 provides the compound of any one of Embodiments 18-21, wherein the ASGPRBM group comprises:

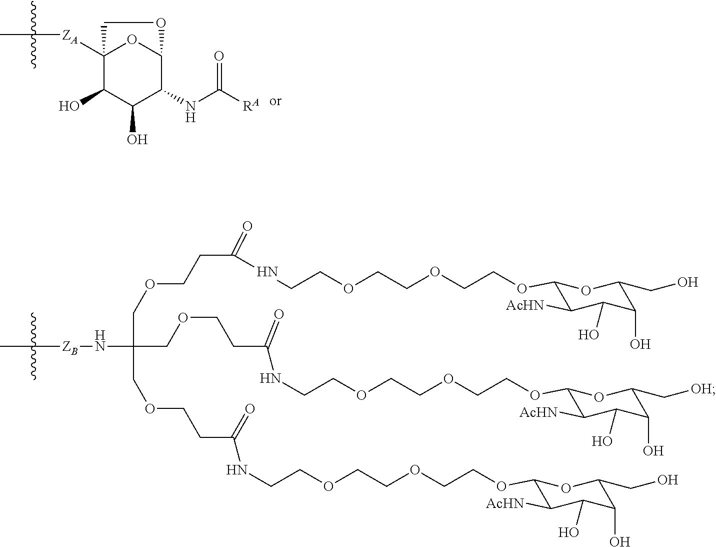

wherein:
R^{A} is C₁-C₃ alkyl optionally substituted with 1-5 independently selected halogens;
Z_{A} is —(CH₂)_{IM}-, —O—(CH₂)_{IM}-, —S—(CH₂)_{IM}-, —NR^{M}—(CH₂)_{IM}-, —C(═O)—(CH₂)_{IM}-, a PEG group containing from 1 to 8 ethylene glycol residues, or —C(0)(CH₂)_{IM}NR^{M}-;
Z_{B} is absent, —(CH₂)_{IM}-, —C(═O)—(CH₂)_{IM}-, or —C(═O)(CH₂)_{IM}—NR^{M}-;
R^{M} is H or C₁-C₃ alkyl optionally substituted with 1-2 hydroxyl groups; and each occurrence of IM is independently 0, 1, 2, 3, 4, 5, or 6;

3

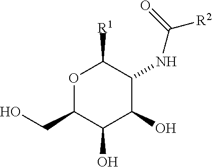

4

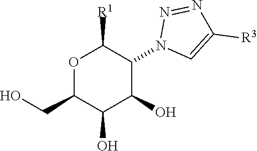

| | R¹ | # | R² |
|---|---|---|---|
| a | Me | 1 | Me |
| b | 4-OMePh | 2 | n-C₂H [symbol] |
| | | 3 | i-Pr |
| | | 4 | t-Bu |
| | | 5 | CCl₃ |
| | | 6 | CF₃ |
| | | 7 | Ot-Bu |
| | | 8 | CH₂CO₂H |
| | | 9 | CH₂NH₂ |
| | | 10 | CH₂CF [symbol] |
| | | 11 | 2-furyl |
| | | 12 | Ph |
| | | 13 | 4-OMePh |
| | | 14 | 3-OMePh |
| | | 15 | 4-CNPh |
| | | 16 | 3-pyridyl |
| | | 17 | 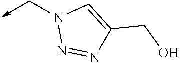 |
| | | # | R³ |
| | | 1 | Ph |
| | | 2 | 4-OMePh |
| | | 3 | 3-OMePh |
| | | 4 | [symbol] -NMe₂Ph |
| | | 5 | 4-NMe₂Ph |
| | | 6 | 2-pyridyl |
| | | 7 | 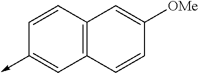 |
| | | 8 | 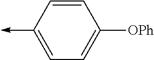 |
| | | 9 | 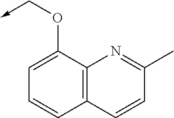 |
| | | 10 | 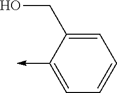 |
| | | 11 | 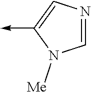 |
| | | 12 | 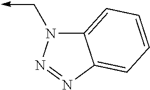 |
| | | 13 | 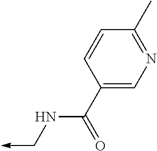 |
| | | 14 | 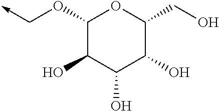 |
| | | 15 | CH₂OH |
| | | 16 | CH₂NH₂ |
| | | 17 | CH₂NHMe |
| | | 18 | CH₂NMe₂ |
| | | 19 | CO₂H |
| | | 20 | CH₂NHCOPh |
| | | 21 | CH₂NHCOMe |

6a-j

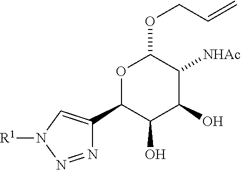

| | R¹ |
|---|---|
| a | Me |
| b | 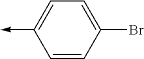 |
| c | 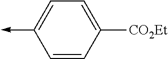 |
| d | 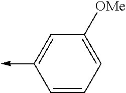 |
| e | 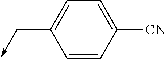 |
| f | 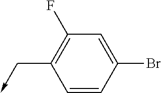 |
| g | 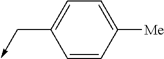 |
| h | 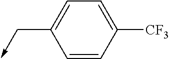 |
| i | 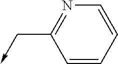 |
| j | 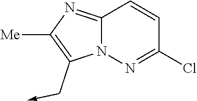 |
| | 8a-j |

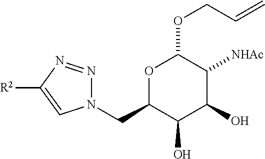

| | R² |
|---|---|
| a | CH₂OH |
| b | 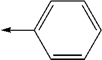 |
| c | 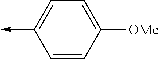 |
| d | 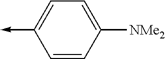 |
| e | 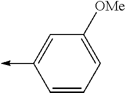 |
| f | 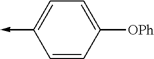 |
| g | 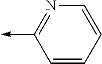 |
| h | 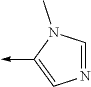 |
| i | 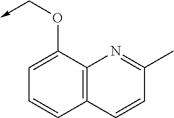 |
| j | 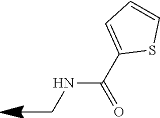 |

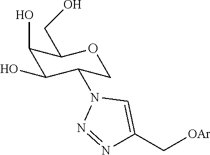

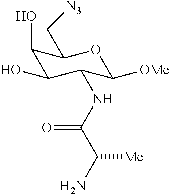 [SMILES: COC1OC(CN=[N+]=[N-])C(O)C(O)C1NC(=O)[C@H](C)N]

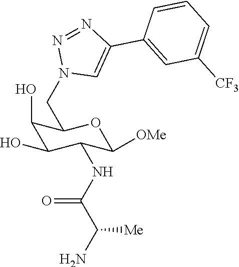 [SMILES: COC1OC(Cn2cc(-c3cccc(C(F)(F)F)c3)nn2)C(O)C(O)C1NC(=O)[C@H](C)N]

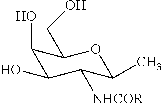

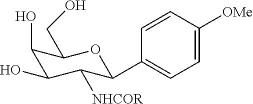

[symbol]

indicates data missing or illegible when filed

wherein
R=CH₃, CF₃, or CH₂CF₃;

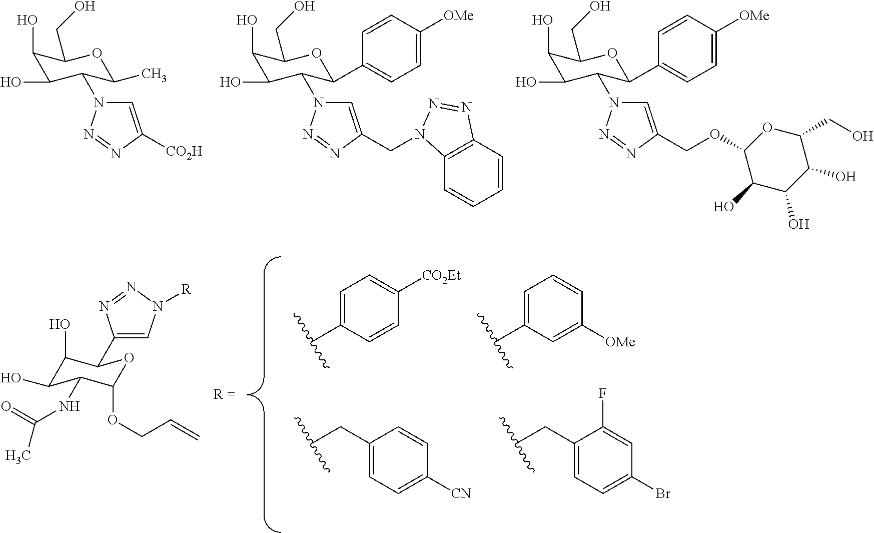

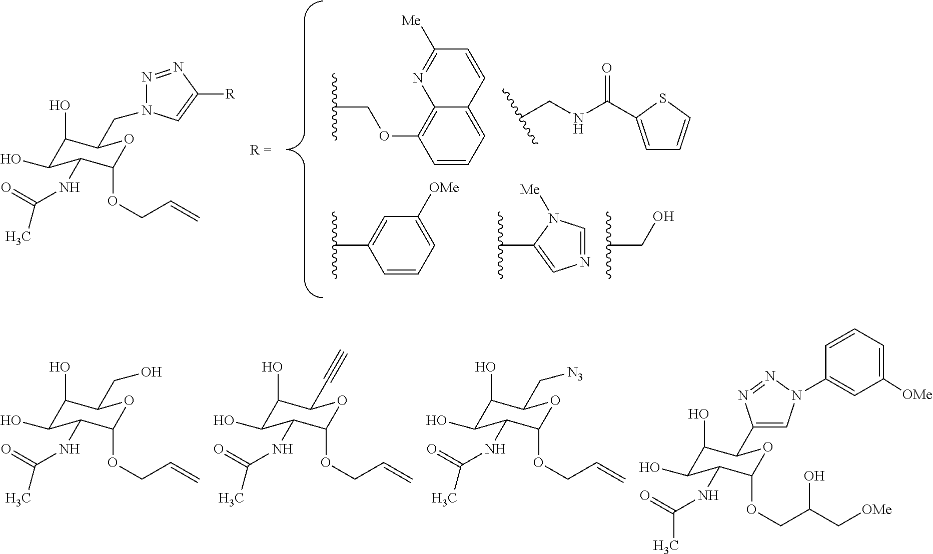

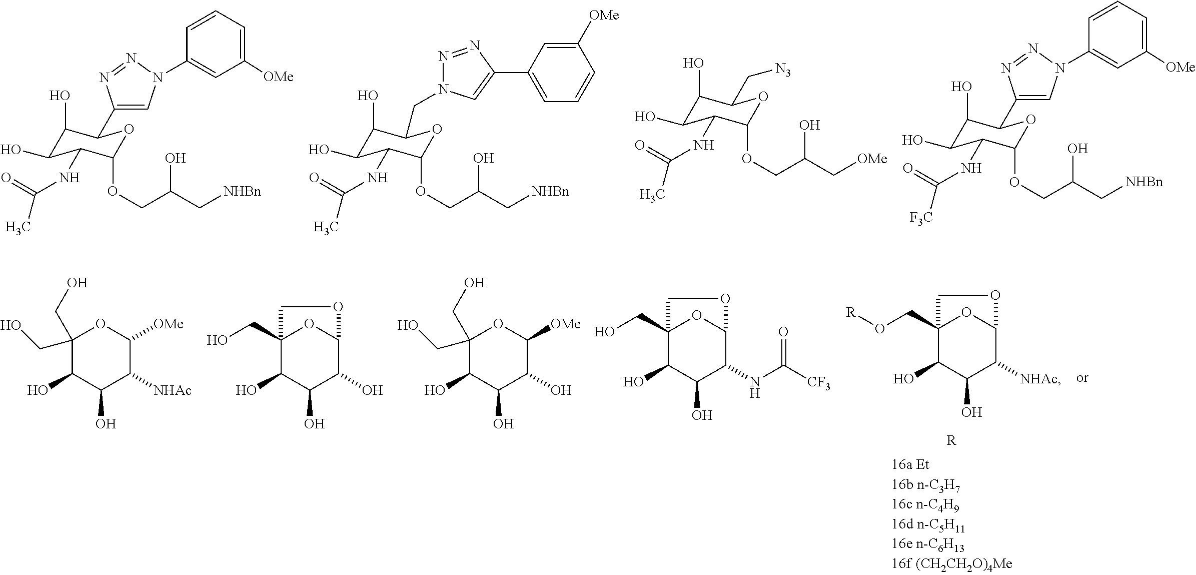

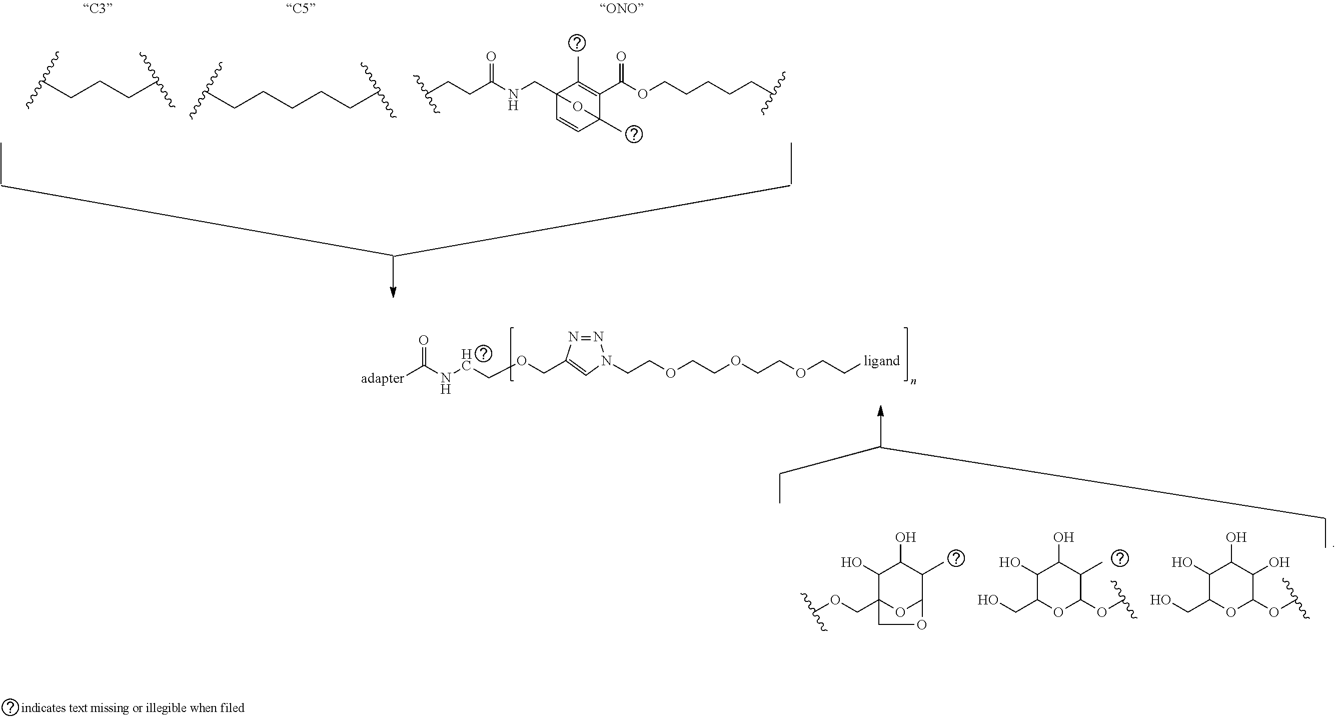

Embodiment 23 provides the compound of any one of Embodiments 1-22, wherein the Linker is a polyethylene glycol containing linker having 1-12 ethylene glycol residues.

Embodiment 24 provides the compound of any one of Embodiments 1-23, wherein the Linker comprises the structure: —CH₂CH₂(OCH₂CH₂)ₘOCH₂—, —(CH₂)ₘCH₂—, -[N(R^{a})—CH(R^{b})(C═O)]ₘ-, or a polypropylene glycol or polypropylene-co-polyethylene glycol group containing 1-100 alkylene glycol units;
wherein each R^{a} is independently H, C₁-C₃ alkyl, or C₁-C₆ alkanol, or combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
wherein m is an integer ranging from 1 to 15; or
   wherein the Linker comprises the structure

-[N(R′—(CH₂)₁₋₁₅—C(═O)]-,
wherein R′ is H or a C₁-C₃ alkyl optionally substituted with 1-2 hydroxyl groups, and m is an integer ranging from 1 to 100; or
   wherein the Linker comprises the structure:

-Z-D-Z′-,
wherein:
   Z and Z′ are each independently a bond, —(CH₂)ᵢ—O—, —(CH₂)ᵢ—S—, —(CH₂)ᵢ—N(R)-,

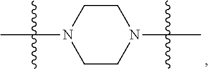 [SMILES: *N1CCN(*)CC1]

—(CH₂)ᵢ—C(R²)═C(R²)-(cis or trans), —(CH₂)ᵢ-═-, or -Y—C(═O)—Y—;
each R is independently H, C₁-C₃ alkyl, or C₁-C₆ alkanol;
each R² is independently H or C₁-C₃ alkyl;
each Y is independently a bond, O, S, or N(R);
each i is independently 0 to 100;
D is a bond, —(CH₂)ᵢ-Y—C(═O)—Y—(CH₂)ᵢ-, —(CH₂)_{m′}-, or —[(CH₂)ₙ-X₁)]ⱼ-, with the
proviso that Z, Z′, and D are not each simultaneously bonds,
X¹ is O, S, or N(R);
j is an integer ranging from 1 to 100;
m′ is an integer ranging from 1 to 100;
n is an integer ranging from 1 to 100; or
   wherein the Linker comprises the structure:

—CH₂—(OCH₂CH₂)ₙ—CH₂—, —(CH₂CH₂O)_{n′}CH₂CH₂—, or —(CH₂CH₂CH₂O)ₙ-,
wherein each n and n′ is independently an integer ranging from 1 to 25; or wherein the Linker comprises a structure:

-PEG-CON-PEG-
wherein each PEG is independently a polyethylene glycol group containing from 1-12 ethylene glycol residues and CON is a triazole group

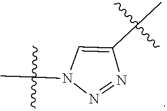 [SMILES: *c1cn(*)nn1]

Embodiment 25 provides the compound of any one of Embodiments 1-24, wherein the CON comprises a structure:

wherein R′ and R″ are each independently H, methyl, or a bond: or

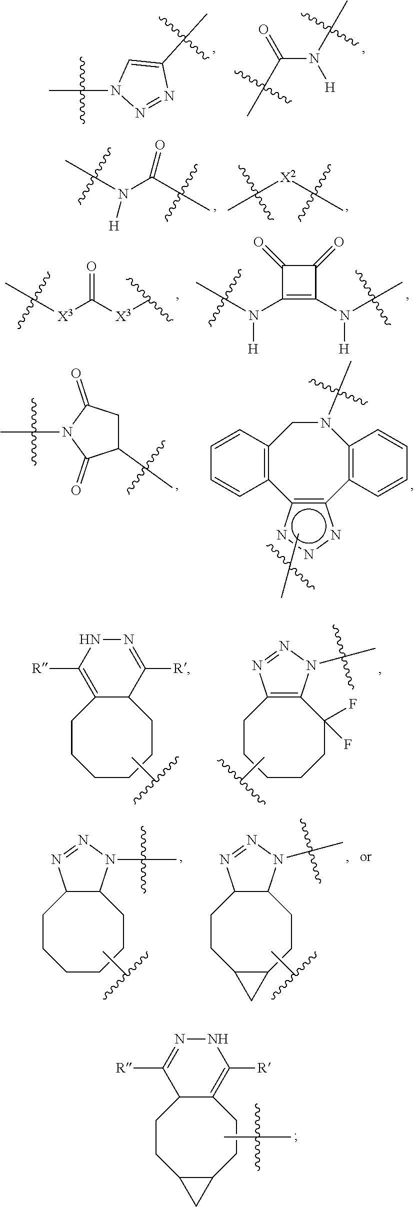

wherein the CON comprises a structure:

—C(═O)—N(R¹)—(CH₂)_{n″}—N(R′)C(═O)—,

—N(R¹)—C(═O)(CH₂)_{n″}—C(═O)N(R¹)—, or

—N(R¹)—C(═O)(CH₂)_{n″}—N(R¹)C(═O)—;
wherein each R′ is independently H or C₁-C₃ alkyl, and n″ is independently an integer
from 0 to 8, in certain embodiments 1 to 7, in certain embodiments 1, 2, 3, 4, 5 or 6; or wherein the CON comprises a structure:

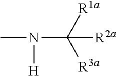

wherein:
R^{1a}, R^{2a} and R^{3a} are each independently H, —(CH₂)_{M1}-, —(CH₂)_{M2}C(═O)_{M3}(NR⁴)_{M3}—(CH₂)_{M2}-, —(CH₂)_{M2}(NR⁴)_{M3}C(O)_{M3}—(CH₂)_{M2}-, or —(CH₂)_{M2}O—(CH₂)_{M1}—C(O)NR⁴—, with the
proviso that R^{1a}, R^{2a} and R^{3a} are not simultaneously H;
each M1 is independently 1, 2, 3, or 4;
each M2 is independently 0, 1, 2, 3, or 4;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0; or
   wherein the CON comprises a structure:

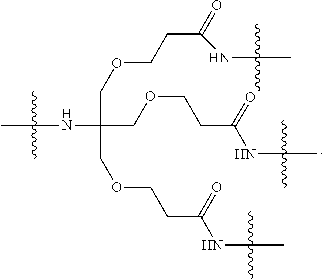

Embodiment 26 provides the compound of any one of Embodiments 1 or 4-25, wherein the Protein binder that binds to CD40L comprises:

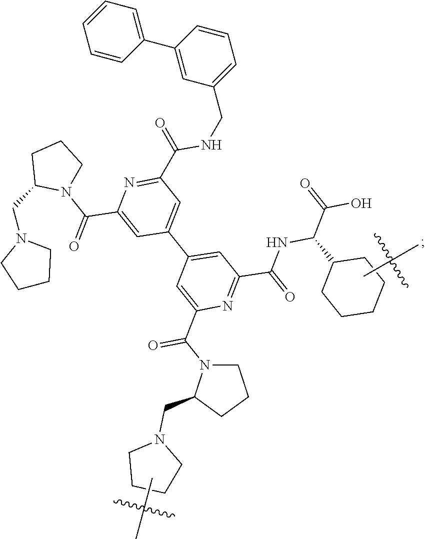

wherein the Protein binder that binds to PCSK9 comprises:

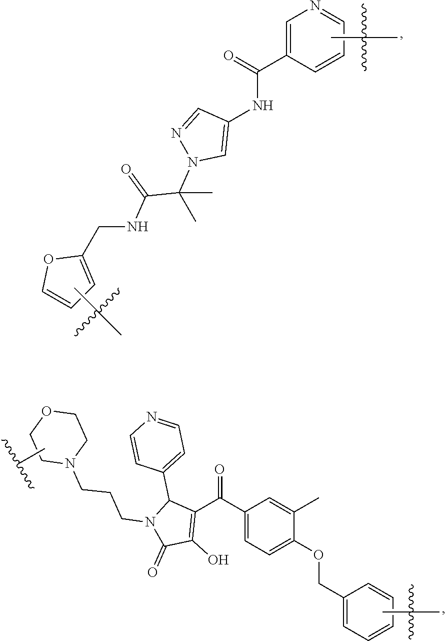

[symbol] NH-TVFTSWEEYLDWV-X (SEQ ID NO:66), wherein X=OH or NH₂;

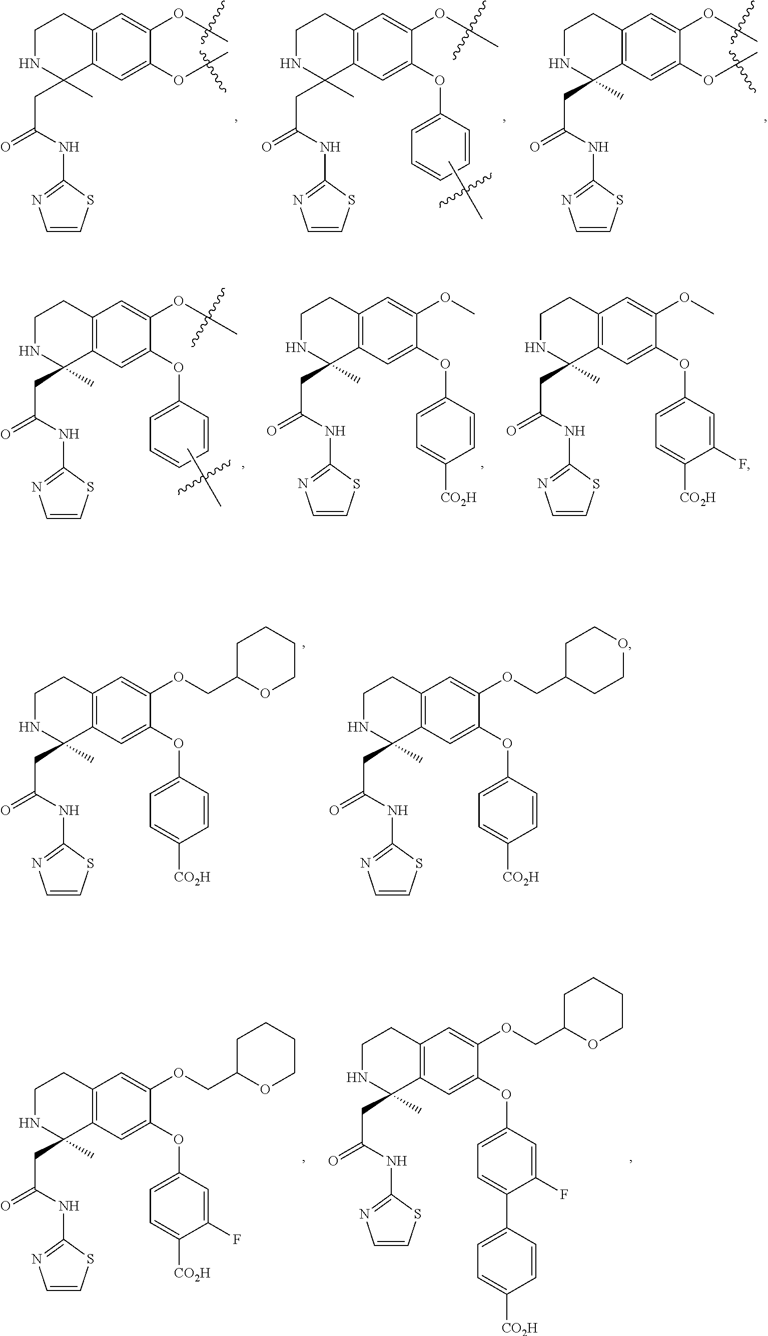

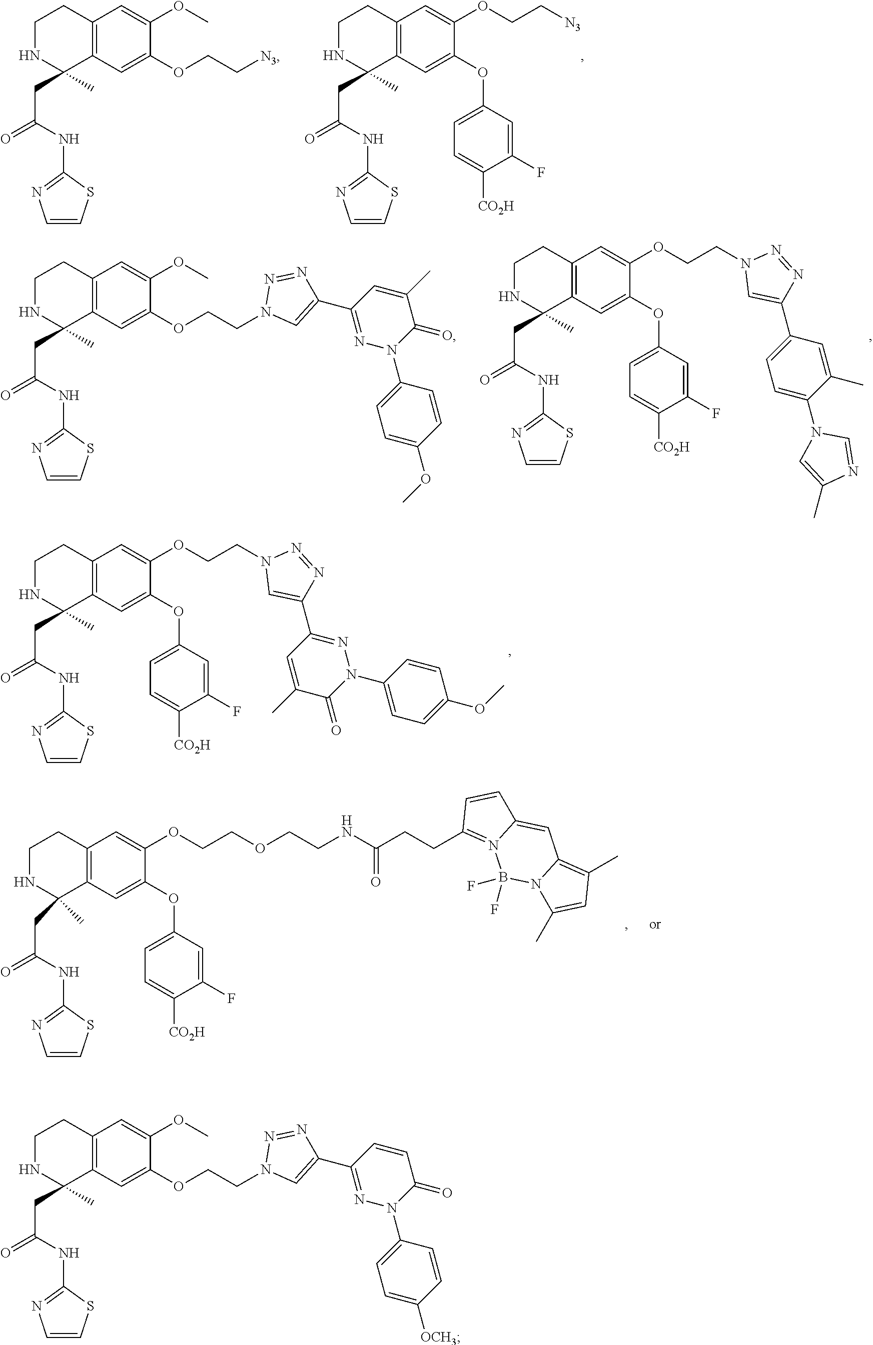

wherein the Protein binder that binds to VEGF comprises:
[symbol] NH-VEPNCDIHVMWEWECFERL-X (SEQ ID NO:67), wherein X=OH or NH₂,

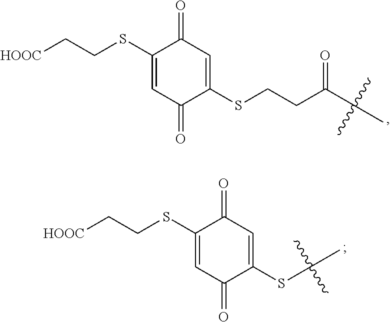 [SMILES: *C(=O)CCSC1=CC(=O)C(SCCC(=O)O)=CC1=O ; *SC1=CC(=O)C(SCCC(=O)O)=CC1=O]

wherein the Protein binder that binds to TGF-beta comprises:
[symbol] NH-KRFKQDGGC-X (SEQ ID NO:68), wherein X=OH or NH₂;

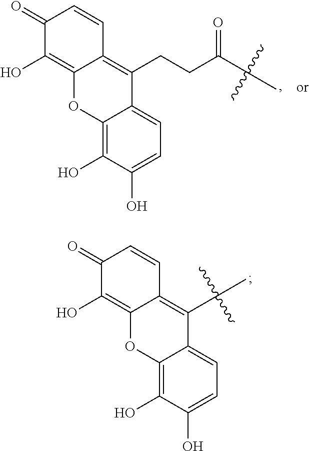

wherein the Protein binder that binds to TSP-1 comprises:
[symbol] NH-RGQILSKLRL-X (SEQ ID NO:69), wherein X=OH or NH₂;
   wherein the Protein binder that binds to soluble uPAR comprises:

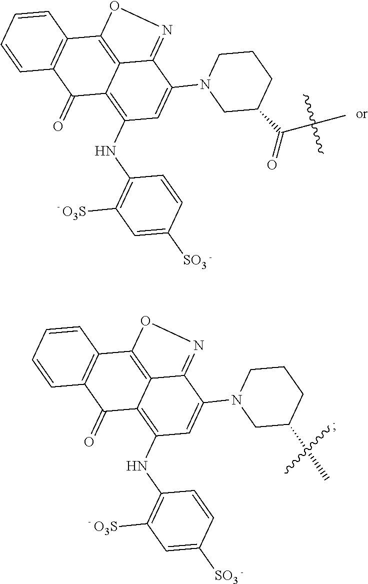

wherein the Protein binder that binds to soluble PSMA comprises:

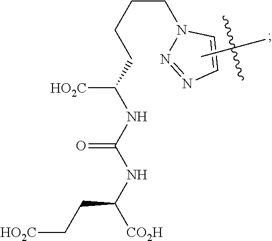

wherein the Protein binder that binds to IL-2 comprises:

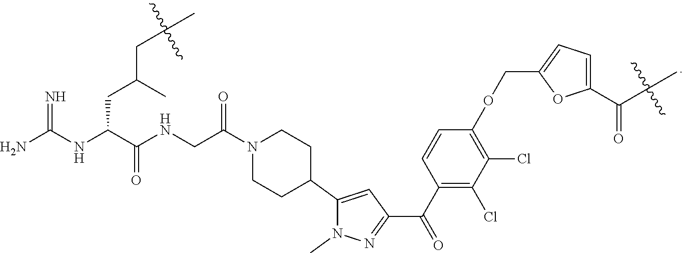 [SMILES: *CC(C)C[C@@H](NC(=N)N)C(=O)NCC(=O)N1CCC(c2cc(C(=O)c3ccc(OCc4ccc(C(*)=O)o4)c(Cl)c3Cl)nn2C)CC1]

wherein the Protein binder that binds to GP120 comprises:

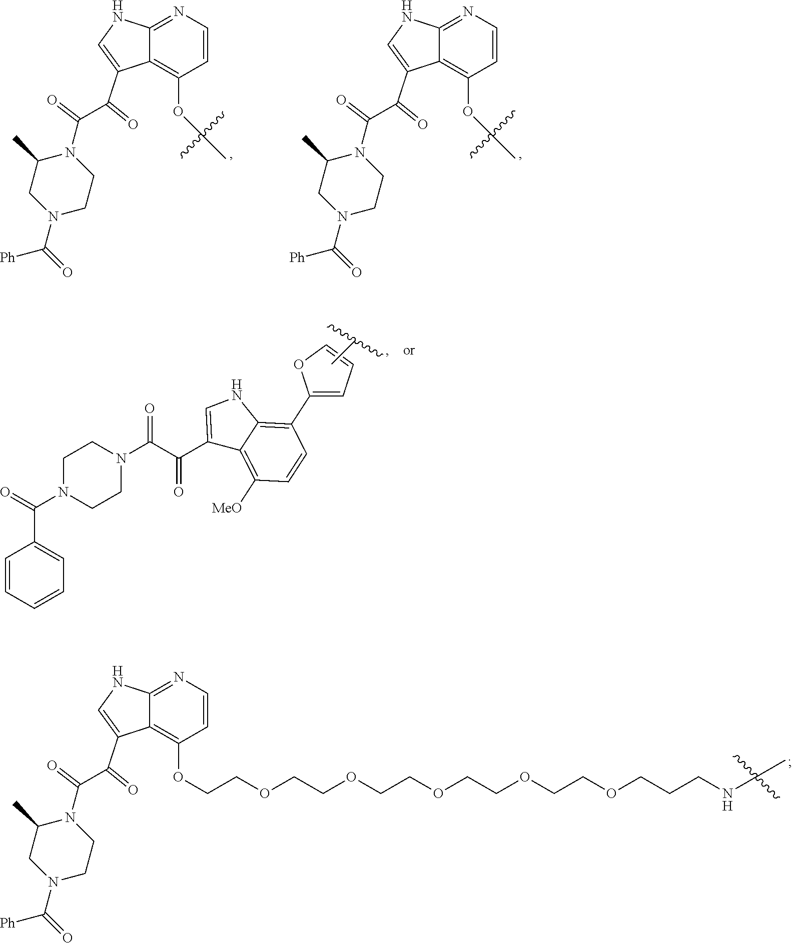

wherein the Protein binder that binds to MIF comprises:

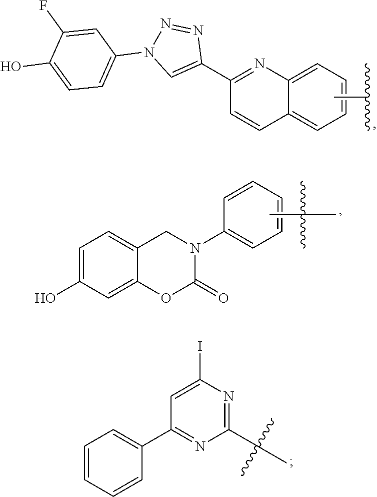

wherein the Protein binder that binds to IgA comprises:

SEQ ID NO: 70)
STFCLLGQKDQSYCFTI
(SEQ ID NO: 71)
HMRCLHYKGRRVCFLL,
(SEQ ID NO: 72)
KTMCLRYNHDKVCFRI,
(SEQ ID NO: 73)
LVLCLVHRTSKHRKCFVI,
A2-3a:
(SEQ ID NO: 75)
SDVCLRYRGRPVCFQV,
Opt-1:
(SEQ ID NO: 76)
HMVCLAYRGRPVCFAL,
Opt-2:
(SEQ ID NO: 77)
HMVCLSYRGRPVCFSL,
Opt-3:
(SEQ ID NO: 78)
HQVCLSYRGRPVCFST,
(SEQ ID NO: 79)
RDVCLRYRGRPVCFQV,
(SEQ ID NO: 80)
HDVCLRYRGRPVCFQV,
(SEQ ID NO: 81)
ADVCLRYRGRPVCFQV,
(SEQ ID NO: 82)
SAVCLRYRGRPVCFQV,
(SEQ ID NO: 83)
SMVCLRYRGRPVCFQV,
(SEQ ID NO: 84)
SDRCLRYRGRPVCFQV,
(SEQ ID NO: 85)
SDACLRYRGRPVCFQV,
(SEQ ID NO: 86)
SDVCARYRGRPVCFQV,
(SEQ ID NO: 87)
SDVCLNYRGRPVCFQV,
(SEQ ID NO: 88)
SDVCLHYRGRPVCFQV,
(SEQ ID NO: 89)
SDVCLAYRGRPVCFQV,
(SEQ ID NO: 90)
SDVCLRARGRPVCFQV,
(SEQ ID NO: 91)
SDVCLRYAGRPVCFQV,
(SEQ ID NO: 92)
SDVCLRYRARPVCFQV,
(SEQ ID NO: 93)
SDVCLRYRGSPVCFQV,
(SEQ ID NO: 94)
SDVCLRYRGAPVCFQV,
(SEQ ID NO: 95)
SDVCLRYRGRRVCFQV,
(SEQ ID NO: 96)
SDVCLRYRGRAVCFQV,
(SEQ ID NO: 97)
SDVCLRYRGRPACFQV,
(SEQ ID NO: 98)
SDVCLRYRGRPVCRQV,
(SEQ ID NO: 99)
SDVCLRYRGRPVCAQV,
(SEQ ID NO: 100)
SDVCLRYRGRPVCFRV,
(SEQ ID NO: 101)
SDVCLRYRGRPVCFLV,
(SEQ ID NO: 102)
SDVCLRYRGRPVCFAV,
(SEQ ID NO: 103)
SDVCLRYRGRPVCFQW,
(SEQ ID NO: 104)
SDVCLRYRGRPVCFQL,
(SEQ ID NO: 105)
SDVCLRYRGRPVCFQA,
(SEQ ID NO: 106)
GRYQCQYRIGHYRFRYSD,
(SEQ ID NO: 107)
GRYQAQYRIGHYRFRYSD,
(SEQ ID NO: 108)
GRYQCQYRIGHYRFRYSD,
(SEQ ID NO: 109)
CLIPS-CHYRFRC,
(SEQ ID NO: 110)
CLIPS-CRIGHYRFRC,
(SEQ ID NO: 111)
CLIPS-YQACHYRFRC,
(SEQ ID NO: 112)
CLIPS-RYQAQCRIGHYRFC,
(SEQ ID NO: 113)
CLIPS-GRYQCQYRIGHYRFRYCD,
(SEQ ID NO: 114)
CLIPS-GRYQACYRIGHYRFRCSD,
(SEQ ID NO: 115)
CLIPS-GRYQAQCRIGHYRFCYSD,
(SEQ ID NO: 116)
RYQAQCRIGHYRFC,
(SEQ ID NO: 117)
GRYQCQYRIGHYRFRYCD,
(SEQ ID NO: 118)
GRYQACYRIGHYRFRCSD,
(SEQ ID NO: 119)
GRYQAQCRIGHYRFCYSD,

each of which can be acyclic or cyclic.

Embodiment 27 provides the compound of any one of Embodiments 2 and 4-25, wherein the TNF binder comprises the amino acid sequence of at least one of:

(SEQ ID NO: 120)
STPTRYS,
(SEQ ID NO: 121)
CALWHWWHC,
(SEQ ID NO: 122)
C(T/S)WLHWWAC,
(SEQ ID NO: 123)
(L/M)HEL(Y/F)(L/M)X(W/Y/F),
(SEQ ID NO: 124)
D-DDDEK QLKER WYKRW LEYLD EFKKN,
(SEQ ID NO: 125)
D-TEEEK QLKEW WYKHW QEYLE EFKKN,
(SEQ ID NO: 126)
GACPPCLWQVLCGGSGSGSG,
(SEQ ID NO: 127)
HIHDDLLRYYGW linear
or
(SEQ ID NO: 128)
tetra branched
peptide,
(SEQ ID NO: 129)
KRWSRYF,
(SEQ ID NO: 127)
HIHDDLLRYYGW,
(SEQ ID NO: 130)
YCWSQYLCY,
(SEQ ID NO: 131)
DELPHYKNTSLGHRP,
and
(SEQ ID NO: 132)
YCLYQSWCY;
or

wherein the TNF binder comprises at least one of:
TNFR1, TNFR2, P51, P52, anticachexin C1, anticachexin C2, adalimumab, infliximab, etanercept, golimumab, certolizumab; or
   wherein the TNF binder comprises at least one of:

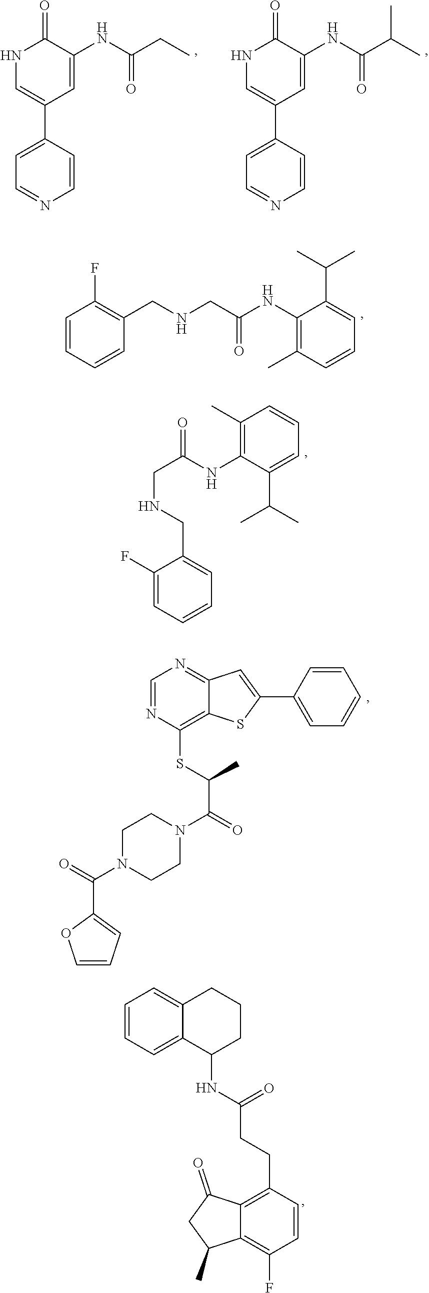

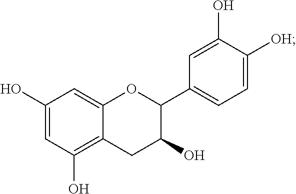 [SMILES: Oc1cc(O)c2c(c1)OC(c1ccc(O)c(O)c1)[C@@H](O)C2]

or
wherein the TNF binder comprises at least one of:

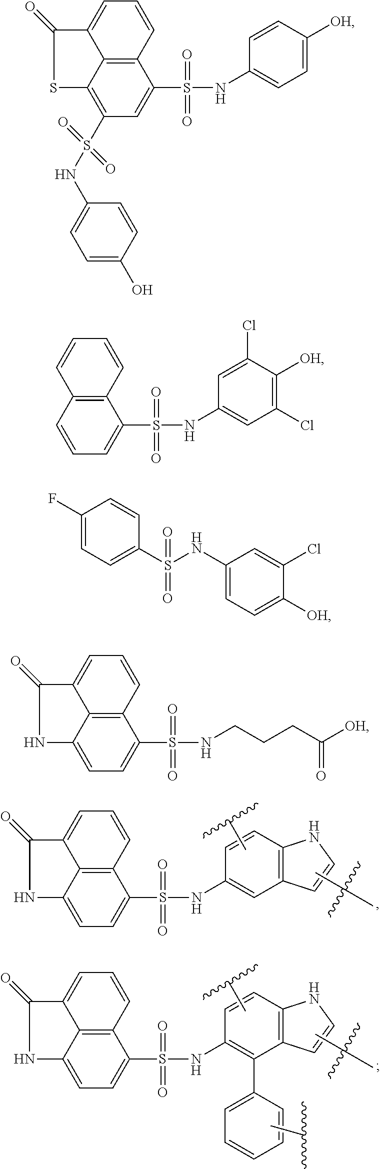

or
wherein the TNF binder comprises at least one of:

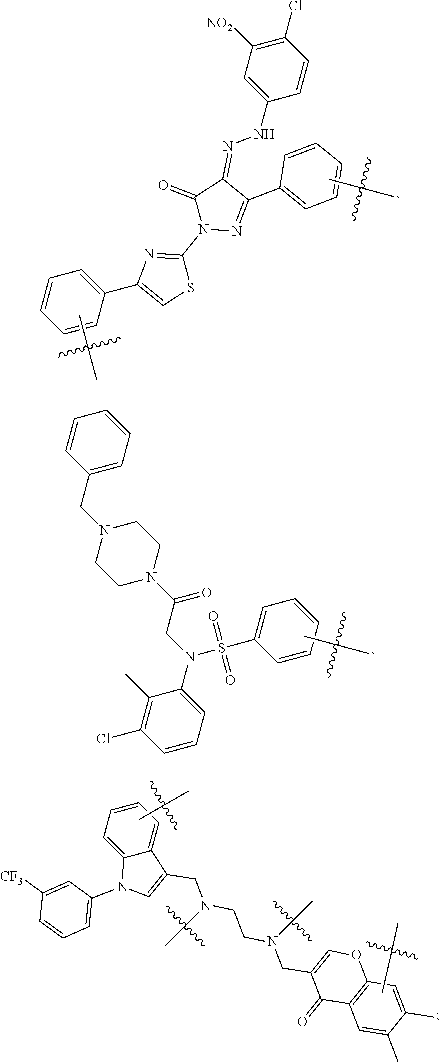

wherein the TNF binder comprises at least one of:

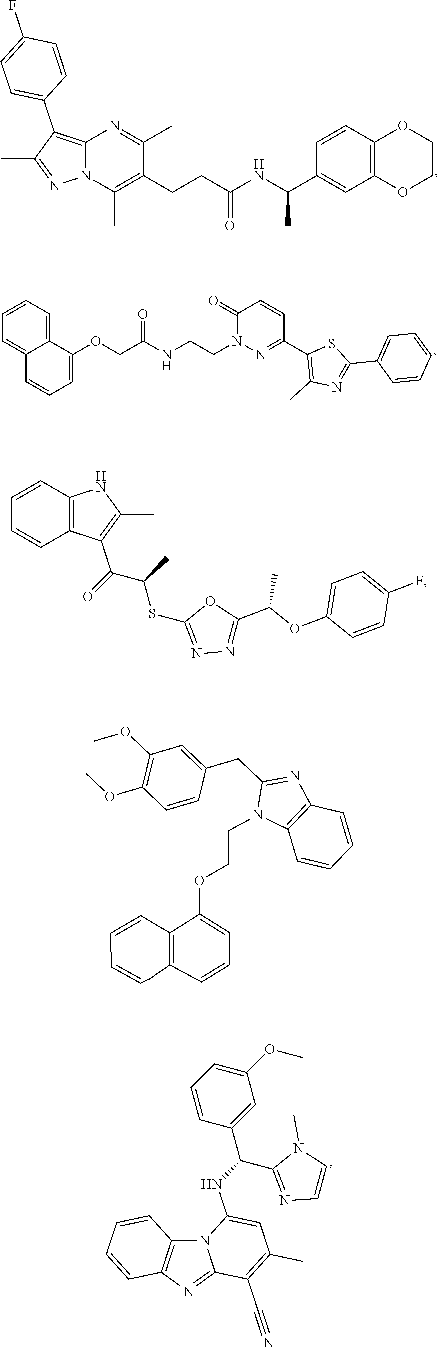

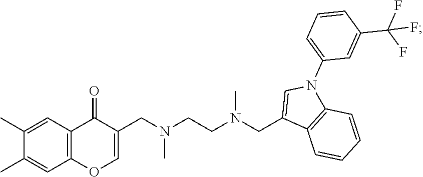 [SMILES: Cc1cc2occ(CN(C)CCN(C)Cc3cn(-c4cccc(C(F)(F)F)c4)c4ccccc34)c(=O)c2cc1C]

wherein the TNF binder comprises at least one of:

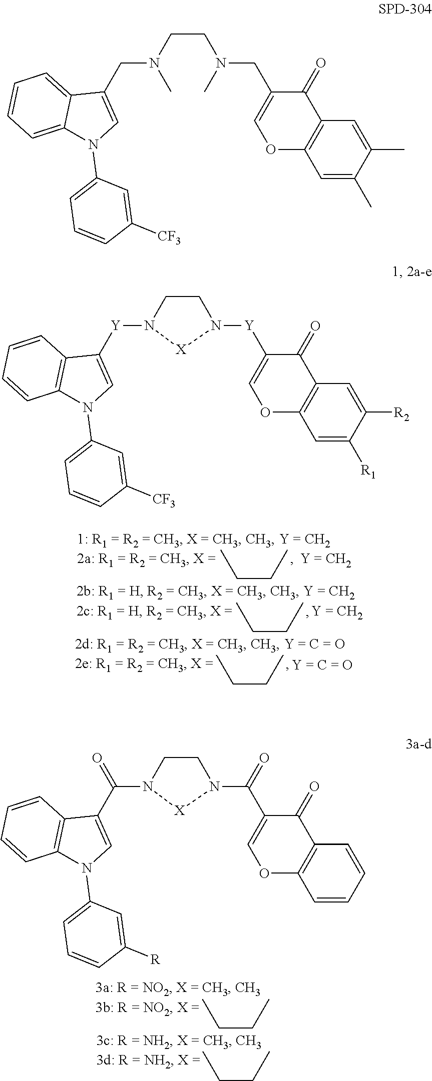

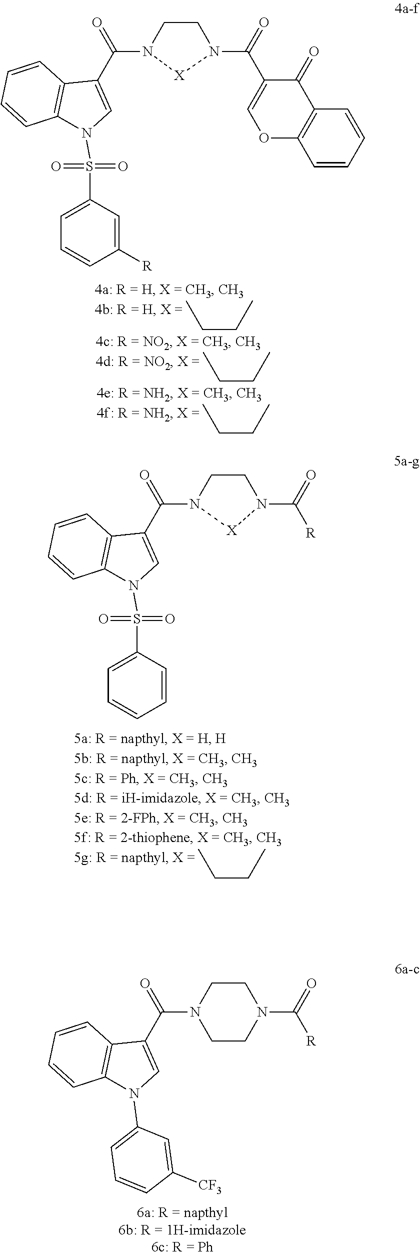

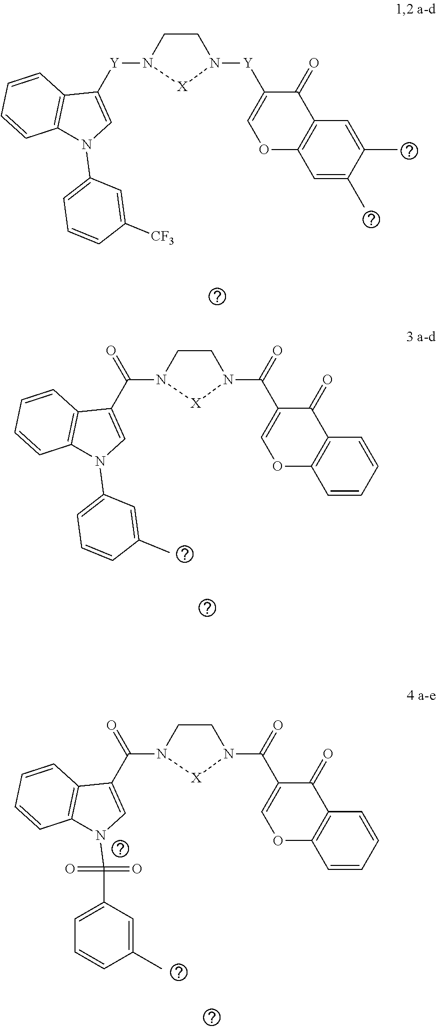

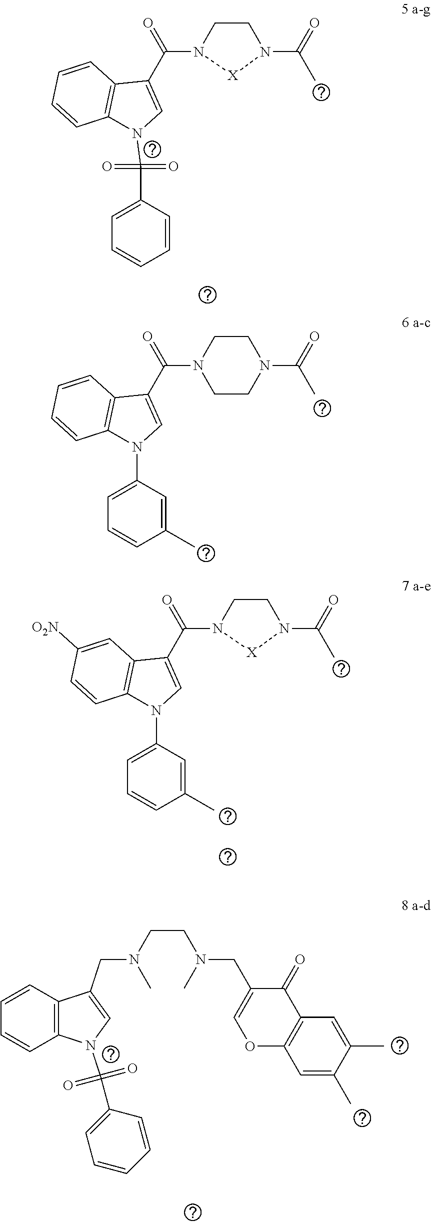

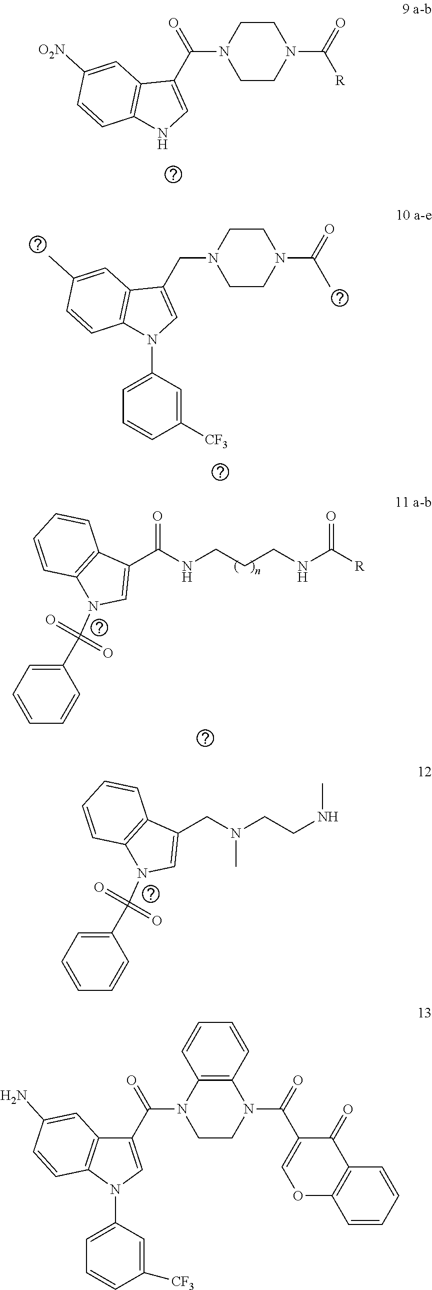

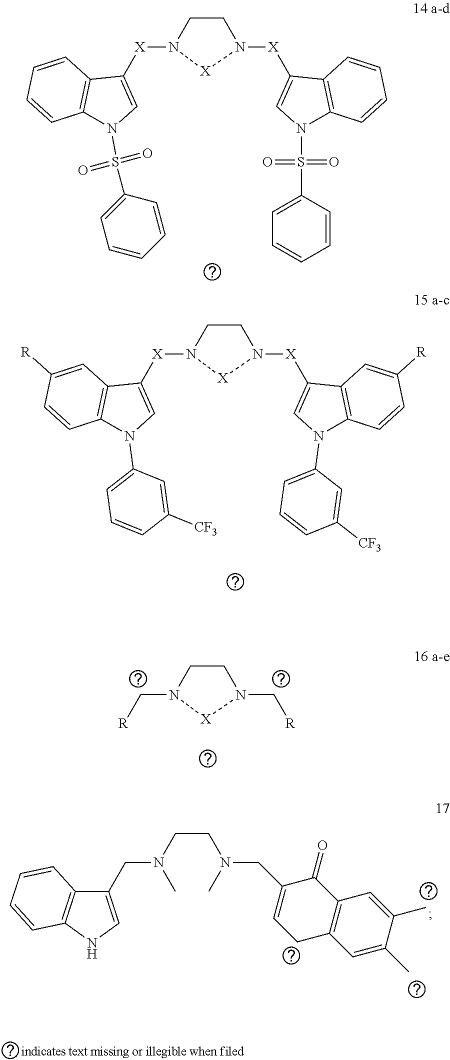

or
wherein the TNF binder comprises at least one of:

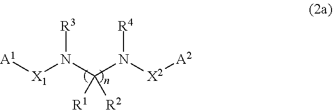

wherein:
A¹ and A² are independently a substituted or unsubstituted phenyl group, wherein the substituents comprise at least one of F, Cl, Br, I, OH, C₁-C₄ alkyl, C₁-C₄ alkyl substituted with at least one OH, C₁-C₄ fluoroalkyl, C₁-C₄ alkoxy, C₁-C₄ haloalkoxy, benzyloxy, and the following heterocyclic rings optionally substituted with at least one of F, Cl, Br, I, OH, C₁-C₄ alkyl, C₁-C₄ alkyl substituted with at least one OH, C₁-C₄ fluoroalkyl, C₁-C₄ alkoxy, and C₁-C₄ haloalkoxy, or

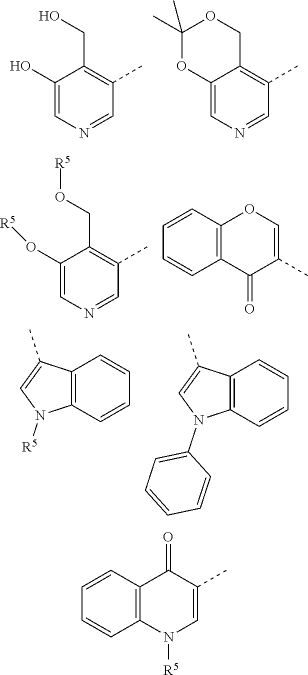

(dotted lines indicate point of attachment);
each R⁵ is independently hydrogen or optionally substituted C₁-C₄ alkyl;
R¹ and R² are independently hydrogen or optionally substituted C₁-C₄ alkyl;
X¹ and X² are independently carbonyl or CH₂;
n is 2, 3, or 4;
R³ and R⁴ are independently hydrogen or optionally substituted C₁-C₄ alkyl, or R³ and R⁴ can combine to form a heterocyclyl ring; or
   wherein the TNF binder comprises at least one of IA-14069,

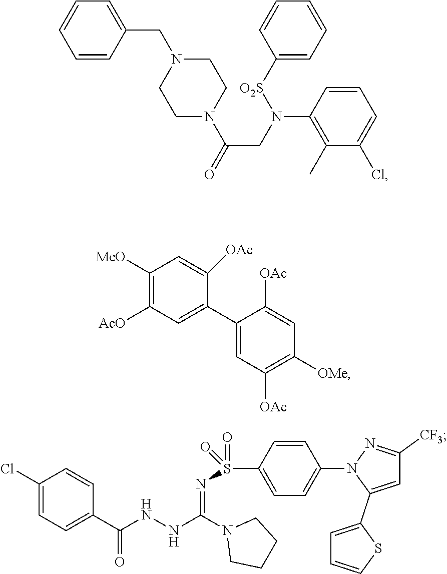

or
wherein the TNF binder comprises at least one of:

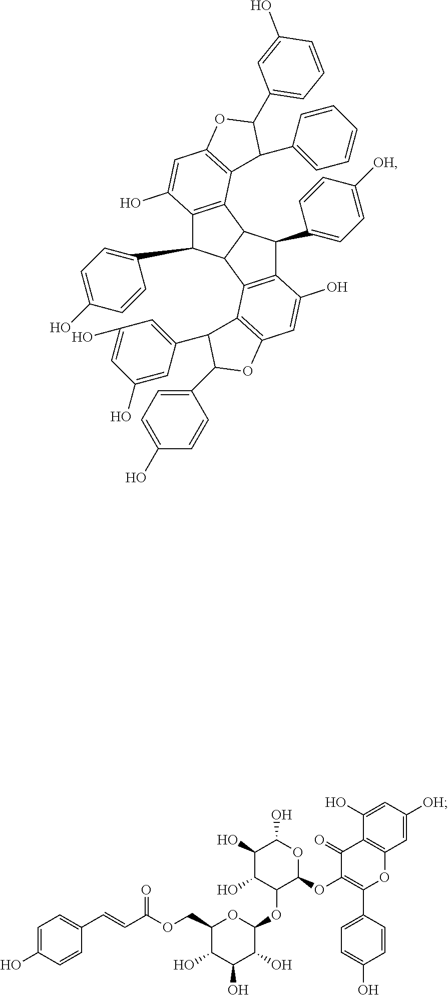 [SMILES: Oc1ccc(C2Oc3cc(O)c4c(c3C2c2cc(O)cc(O)c2)C2C(c3c5c(cc(O)c3[C@@H]2c2ccc(O)cc2)OC(c2cccc(O)c2)C5c2ccccc2)[C@H]4c2ccc(O)cc2)cc1 ; O=C(/C=C/c1ccc(O)cc1)OC[C@H]1O[C@@H](OC2[C@H](Oc3c(-c4ccc(O)cc4)oc4cc(O)cc(O)c4c3=O)O[C@@H](O)[C@H](O)[C@H]2O)[C@H](O)[C@@H](O)[C@@H]1O]

or
wherein the TNF binder comprises at least one of:

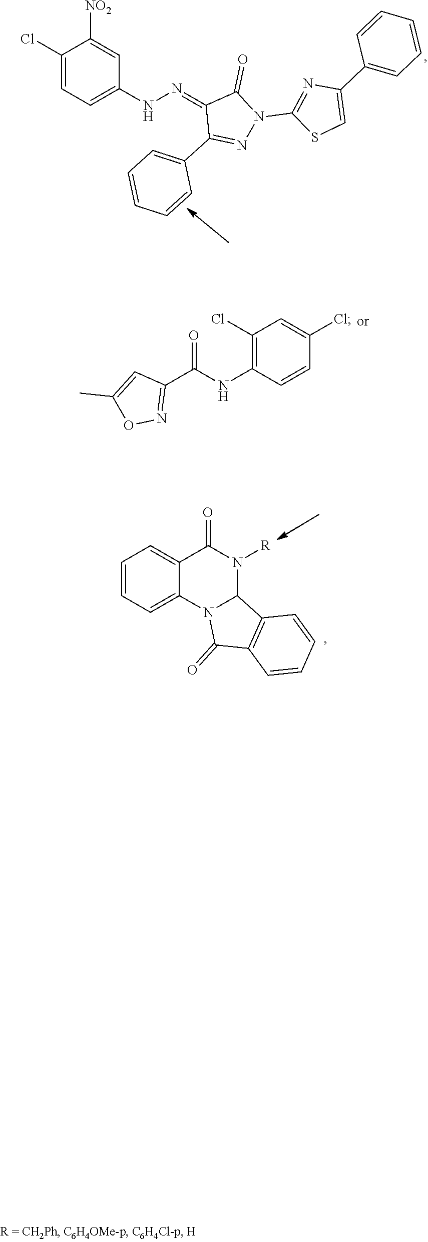

wherein the TNF binder comprises at least one of:

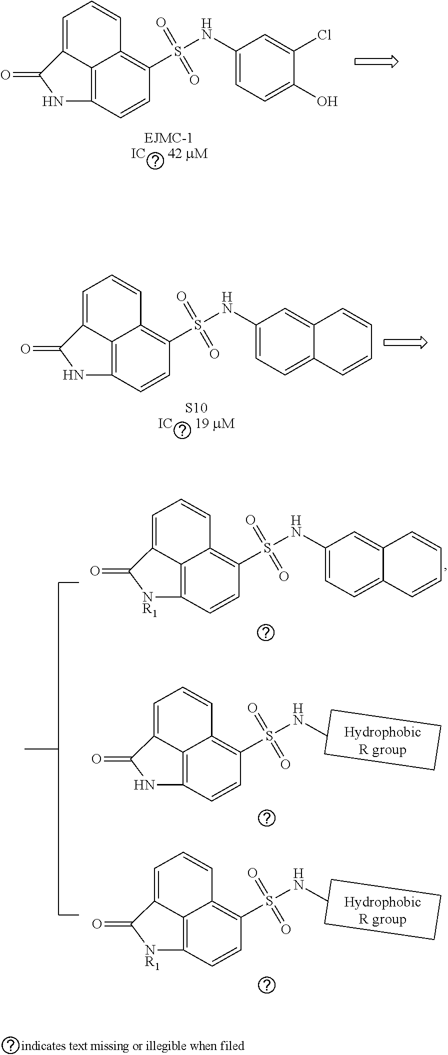

| Com- | |
|---|---|
| pound | Structure |
| EJMC-1 | 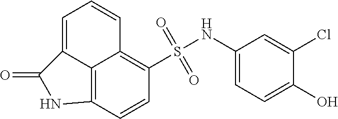 [SMILES: O=C1Nc2ccc(S(=O)(=O)Nc3ccc(O)c(Cl)c3)c3cccc1c23] |
| S10 | 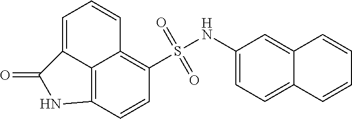 [SMILES: O=C1Nc2ccc(S(=O)(=O)Nc3ccc4ccccc4c3)c3cccc1c23] |
| S21 | 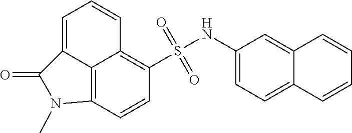 [SMILES: CN1C(=O)c2cccc3c(S(=O)(=O)Nc4ccc5ccccc5c4)ccc1c23] |
| S22 | 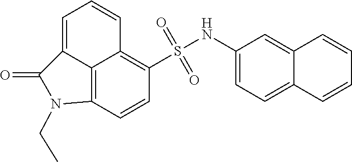 [SMILES: CCN1C(=O)c2cccc3c(S(=O)(=O)Nc4ccc5ccccc5c4)ccc1c23] |
| S23 | 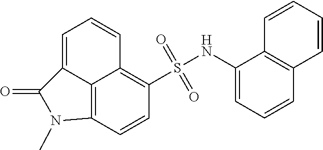 [SMILES: CN1C(=O)c2cccc3c(S(=O)(=O)Nc4cccc5ccccc45)ccc1c23] |
| S24 | 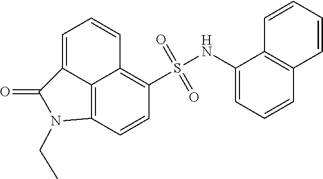 [SMILES: CCN1C(=O)c2cccc3c(S(=O)(=O)Nc4cccc5ccccc45)ccc1c23] |
| S25 | 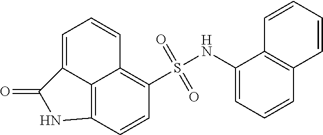 [SMILES: O=C1Nc2ccc(S(=O)(=O)Nc3cccc4ccccc34)c3cccc1c23] |
| S26 | 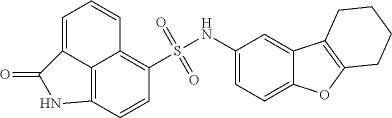 [SMILES: O=C1Nc2ccc(S(=O)(=O)Nc3ccc4oc5c(c4c3)CCCC5)c3cccc1c23] |
| S27 | 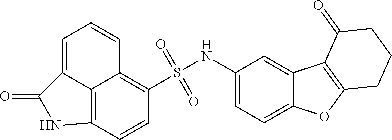 [SMILES: O=C1CCCc2oc3ccc(NS(=O)(=O)c4ccc5c6c(cccc46)C(=O)N5)cc3c21] |
| 4a | 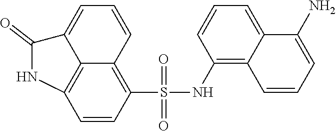 [SMILES: Nc1cccc2c(NS(=O)(=O)c3ccc4c5c(cccc35)C(=O)N4)cccc12] |
| 4c | 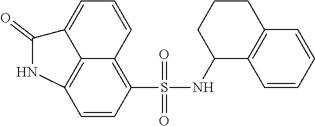 [SMILES: O=C1Nc2ccc(S(=O)(=O)NC3CCCc4ccccc43)c3cccc1c23] |
| 4d | 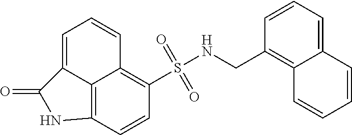 [SMILES: O=C1Nc2ccc(S(=O)(=O)NCc3cccc4ccccc34)c3cccc1c23] |
| 4e | 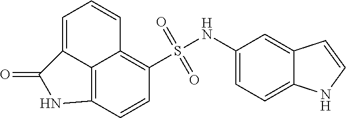 [SMILES: O=C1Nc2ccc(S(=O)(=O)Nc3ccc4[nH]ccc4c3)c3cccc1c23] |
| 4f | 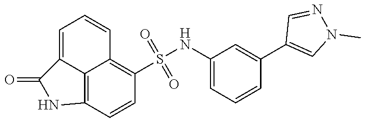 [SMILES: Cn1cc(-c2cccc(NS(=O)(=O)c3ccc4c5c(cccc35)C(=O)N4)c2)cn1] |
| 4g | 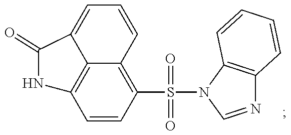 [SMILES: O=C1Nc2ccc(S(=O)(=O)n3cnc4ccccc43)c3cccc1c23] |

or
wherein the TNF binder comprises at least one of:

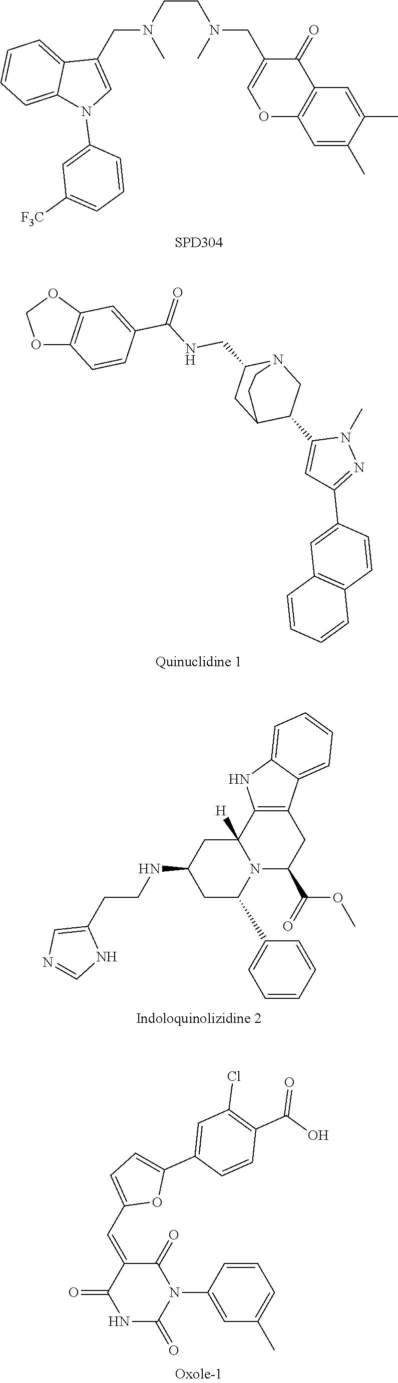 [SMILES: Cc1cc2occ(CN(C)CCN(C)Cc3cn(-c4cccc(C(F)(F)F)c4)c4ccccc34)c(=O)c2cc1C ; "SPD304" ; Cn1nc(-c2ccc3ccccc3c2)cc1[C@H]1CN2CCC1C[C@@H]2CNC(=O)c1ccc2c(c1)OCO2 ; COC(=O)[C@@H]1Cc2c([nH]c3ccccc23)[C@H]2C[C@H](NCCc3cnc[nH]3)C[C@@H](c3ccccc3)N21 ; Cc1cccc(N2C(=O)NC(=O)/C(=C/c3ccc(-c4ccc(C(=O)O)c(Cl)c4)o3)C2=O)c1]

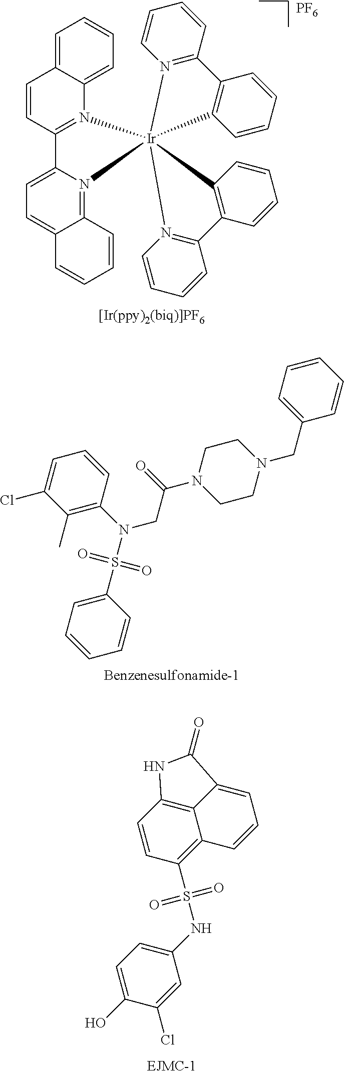

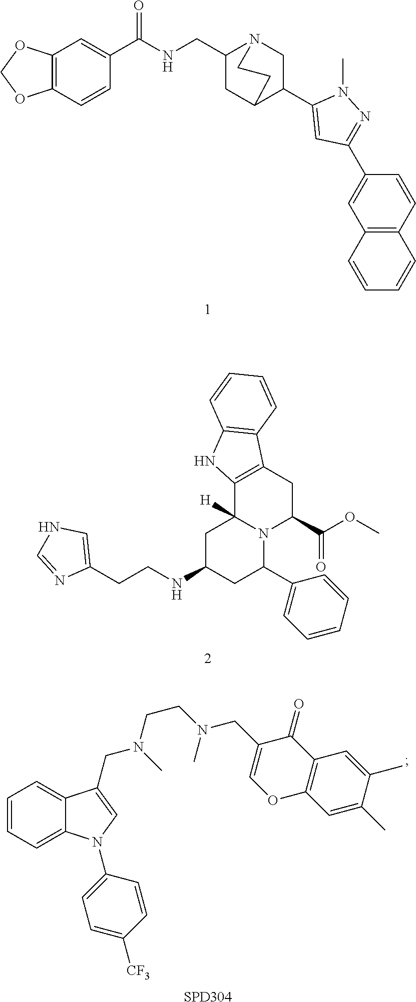 [SMILES: Cn1nc(-c2ccc3ccccc3c2)cc1C1CN2CCC1CC2CNC(=O)c1ccc2c(c1)OCO2 ; COC(=O)[C@@H]1Cc2c([nH]c3ccccc23)[C@H]2C[C@H](NCCc3c[nH]cn3)CC(c3ccccc3)N21 ; Cc1cc2occ(CN(C)CCN(C)Cc3cn(-c4ccc(C(F)(F)F)cc4)c4ccccc34)c(=O)c2cc1C ; "SPD304"]

or
wherein the TNF binder comprises at least one of:

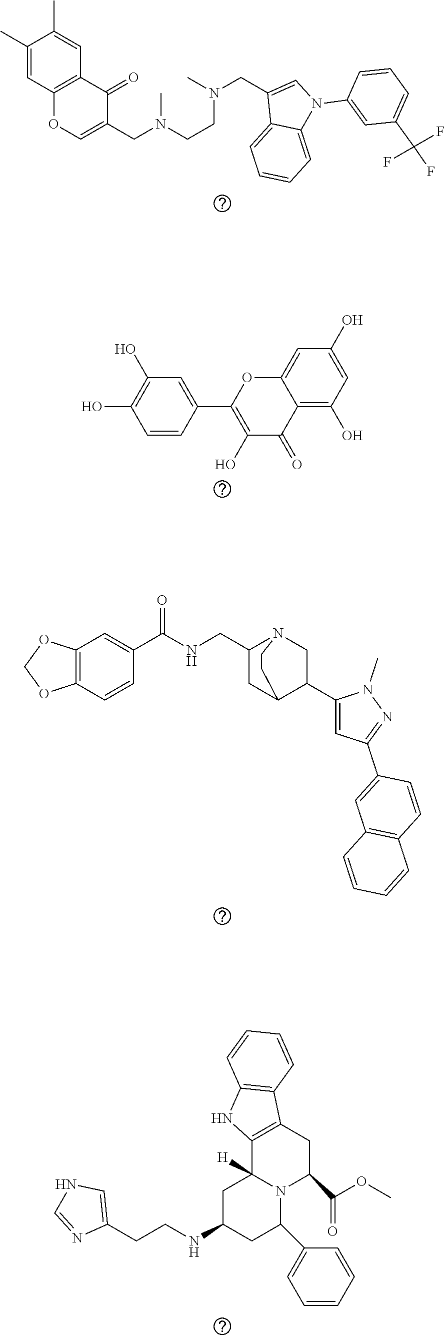 [SMILES: Cc1cc2occ(CN(C)CCN(C)Cc3cn(-c4cccc(C(F)(F)F)c4)c4ccccc34)c(=O)c2cc1C ; O=c1c(O)c(-c2ccc(O)c(O)c2)oc2cc(O)cc(O)c12 ; Cn1nc(-c2ccc3ccccc3c2)cc1C1CN2CCC1CC2CNC(=O)c1ccc2c(c1)OCO2 ; COC(=O)[C@@H]1Cc2c([nH]c3ccccc23)[C@H]2C[C@H](NCCc3c[nH]cn3)CC(c3ccccc3)N21]

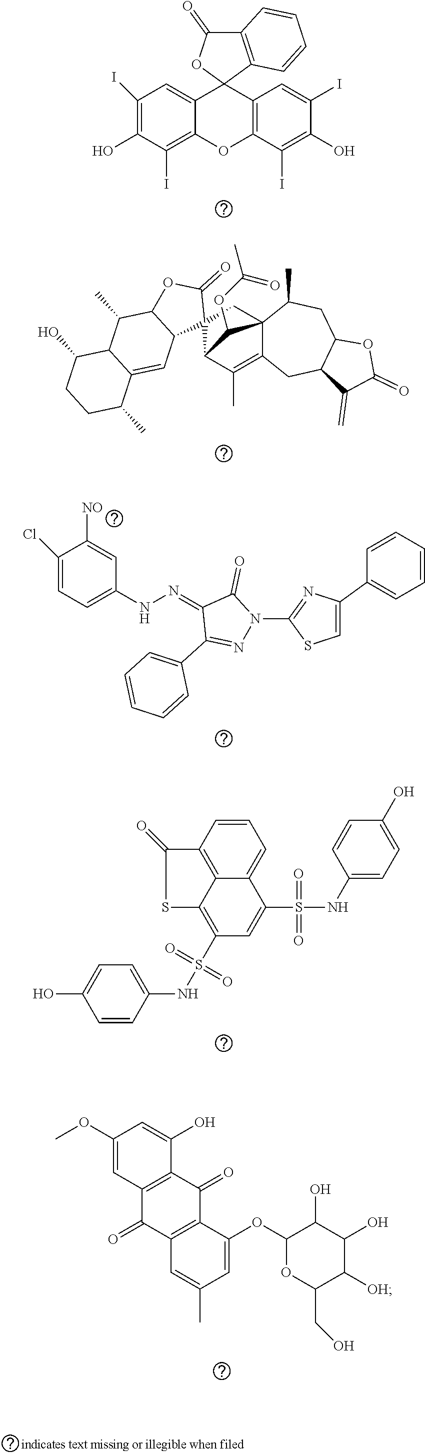

or
wherein the TNF binder comprises at least one of:

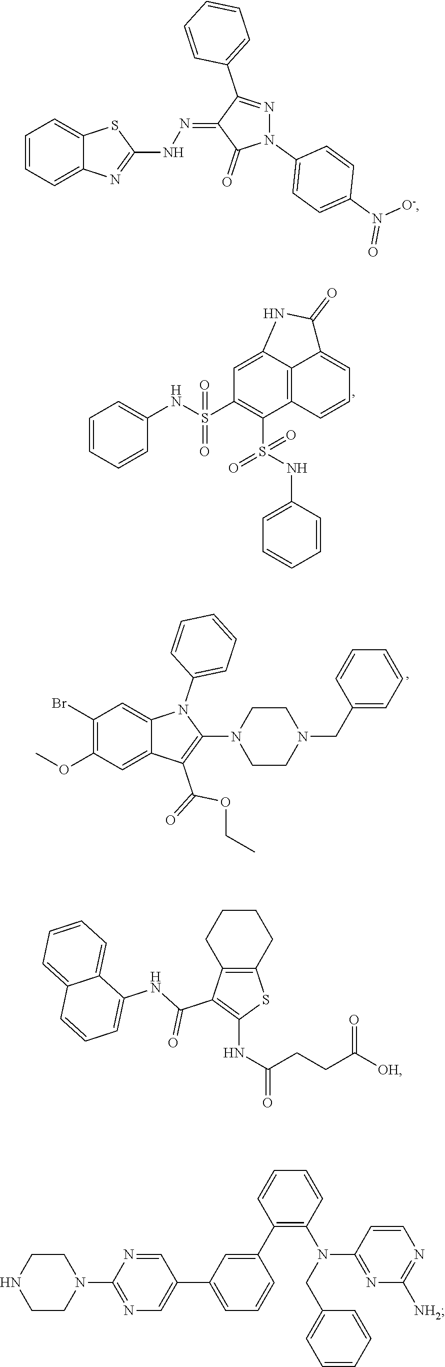

or
wherein the TNF binder comprises at least one of:

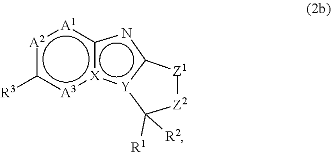

wherein:
R¹ is H, OH, F or optionally substituted (C₁-C₃)alkyl;
R² is optionally substituted aryl, optionally substituted (C₃-C₅)cycloalkyl, optionally substituted heteroaryl or optionally substituted heterocyclyl; or
R¹ and R² together can form an optionally substituted saturated or partially saturated carbocyclic ring or optionally substituted saturated or partially saturated heterocyclic ring;
up to two of A¹, A², and A³ are N, and the rest are independently C(R^{A2}); X is N and Y is C, wherein:
Z¹ is —C(R^{z})₂— and Z² is —C(R^{z})₂—, —N(R^{z1})— or —O—; or
Z¹ is —CH₂— and Z² is —Z^{2a}—Z^{2b}—,
wherein Z^{2a} is attached to Z¹ and Z^{2b} is attached to C(R¹)(R²); and Z^{2a} and Z^{2b} are independently —C(R^{z})₂—, —C(R^{z})₂C(R^{z})₂—, —O— or —N(R^{z})— provided that one of
Z^{2a} and Z^{2b} is —C(R^{z})₂— or —C(R^{z})₂C(R)₂—; or —Z^{2a}—Z^{2b}— form —N(R^{z1})C(O)— or —C(O)N(R^{z1})-;
or
X is C and Y is N, provided that R′ is not —OH or —F, wherein:
Z¹ is —C(R^{z})₂— and Z² is —C(R^{z})₂—; or
Z¹ is —C(R^{z})₂— and Z² is —Z^{2a}—Z^{2b}—,
wherein Z^{2a} is attached to Z¹ and Z^{2b} is attached to C(R¹)(R²), and Z^{2a} is —C(R^{z})₂—, —C(R^{z})₂C(R^{z})₂—, —O— or —N(R^{z1}), and Z^{2b} is —C(R^{z})₂—, or —Z^{2a}—Z^{2b}— form —N(R^{z1})C(O)— or —C(O)N(R^{z1})—;
R³ is —R^{3a}—R^{3b}, wherein:
R^{3a} is an optionally substituted aryl, optionally substituted saturated or partially saturated heterocyclyl or optionally substituted heteroaryl;
R^{3b} is H, —CF₃, —CN, —C(O)OH, —N(R^{a})(R^{b}), —C(O)N(R^{a})R^{b}), —C(O)-optionally substituted heterocyclyl, —O(R^{a}), —S(O)₂(C₁-C₃)alkyl, —S(O)₂N(R_{)(R}^{d}), —S—(C₁-C₃)alkyl, —S(O)₂—R^{C} optionally substituted (C₁-C₅)alkyl, —CH₂)ₚ-optionally substituted (C₃-C₆)cycloalkyl, —CH₂)ₚ-optionally substituted heteroaryl or —(CH₂)ₚ-optionally substituted saturated, unsaturated or partially saturated heterocyclyl; provided that R^{3b} is not H or methoxy when R² is optionally substituted phenyl;
R^{a} and R^{b} are independently selected from H, optionally substituted (C₁-C₅)alkyl, —C(O)-optionally substituted (C₁-C₅)alkyl, optionally substituted —(CH₂)ₚ-(C₁-C₆)cycloalkyl and —(CH₂)ₚ-optionally substituted heterocyclyl;
R^{c} and R^{d} are independently selected from H, optionally substituted (C₁-C₅)alkyl, optionally substituted —(CH₂)ₚ-(C₃-C₆)cycloalkyl and —(CH₂)ₚ-optionally substituted heterocyclyl;
R^{A2} is independently H, CF₃, halo or (C₁-C₃)alkyl;
R^{z} is independently H, F, CF₃, —OH or (C₁-C₃)alkyl;
R^{z1} is independently H or (C₁-C₃)alkyl; and
p is independently 0, 1 or 2; or
   wherein the TNF binder comprises at least one of:

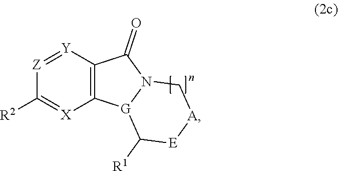

wherein:
X, Y, and Z are independently CR or N; provided that Y and Z are not both N;
A is —C(R^{z})₂—;
E is CH₂ or O and G is CH; or E is CH₂ and G is CH or N;
R¹ is optionally substituted aryl or optionally substituted heteroaryl;
R² is —R^{2a}—R^{2b}, wherein:
R^{2a} is an optionally substituted saturated, unsaturated or partially saturated heterocyclyl or optionally substituted heteroaryl;
R^{2b} is —N(R^{a})(R^{b}), —O(R^{a}), optionally substituted (C₁-C₅)alkyl, optionally substituted (C₃-C₆)cycloalkyl, —(CH₂)ₚ-optionally substituted heteroaryl or —(CH₂)ₚ-optionally substituted heterocyclyl; wherein
R^{a} and R^{b} are independently selected from H, optionally substituted (C₁-C₅)alkyl, and —(CH₂)ₙ-optionally substituted heterocyclyl;
R⁴ is independently H, halo, CF₃, or (C₁-C₃)alkyl;
R^{z} is independently H, halo, CF₃, or (C₁-C₃)alkyl;
n is 0 or 1; and
p is 0 or 1.

Embodiment 28 provides the compound of Embodiment 27, wherein the compound of formula (1a) comprises one of the following:

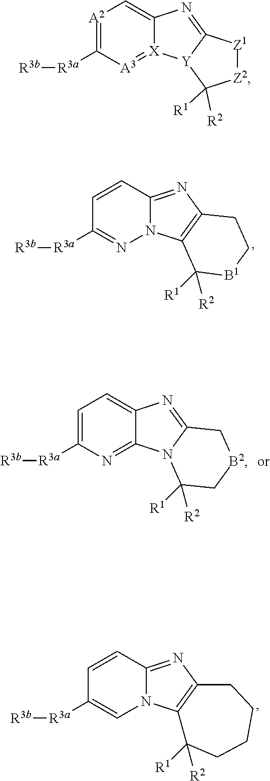

wherein:
A² is CH or N; A³ is CH or N; B¹ is CH₂ or O; B² is CH₂ or O; X is C or N; Y is C or N;
Z¹ is CH₂ or O; and Z² is CH₂ or O;
R^{3a} is selected from the group consisting of:

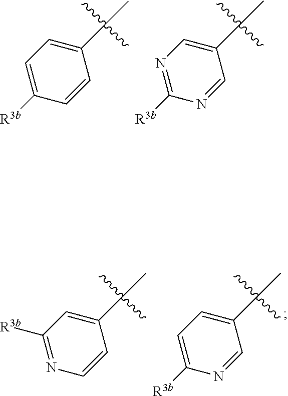

R^{3b} is selected from the group consisting of:

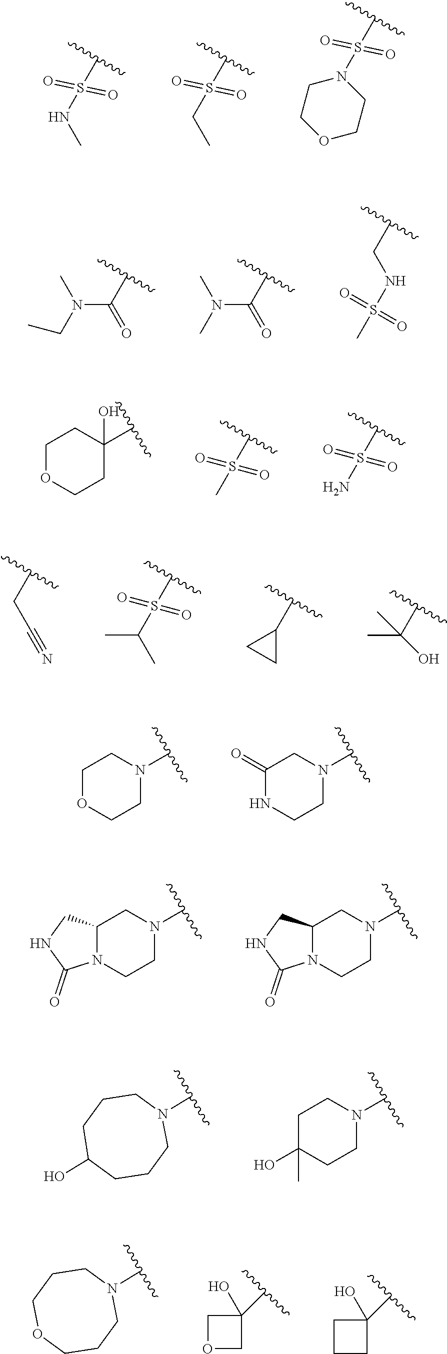

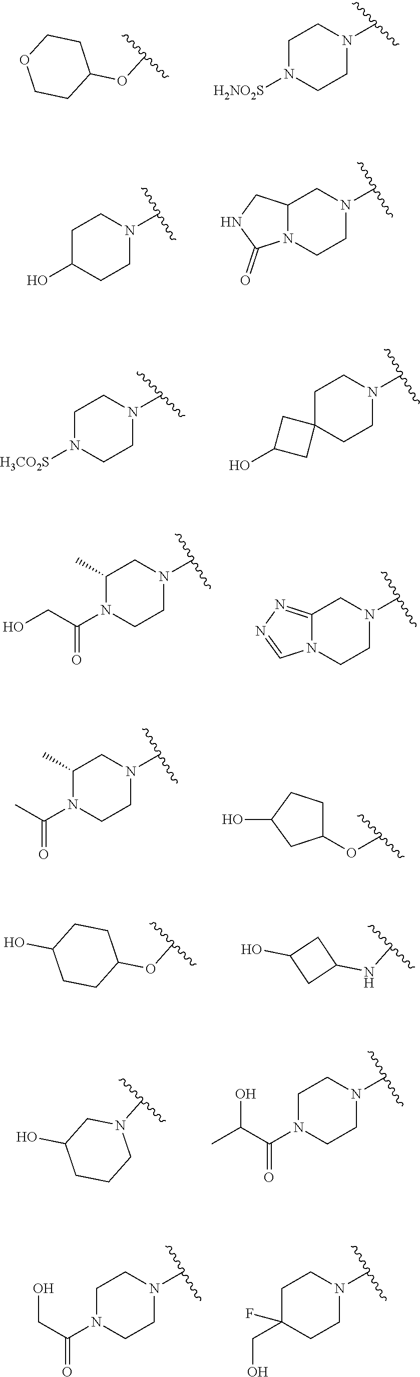

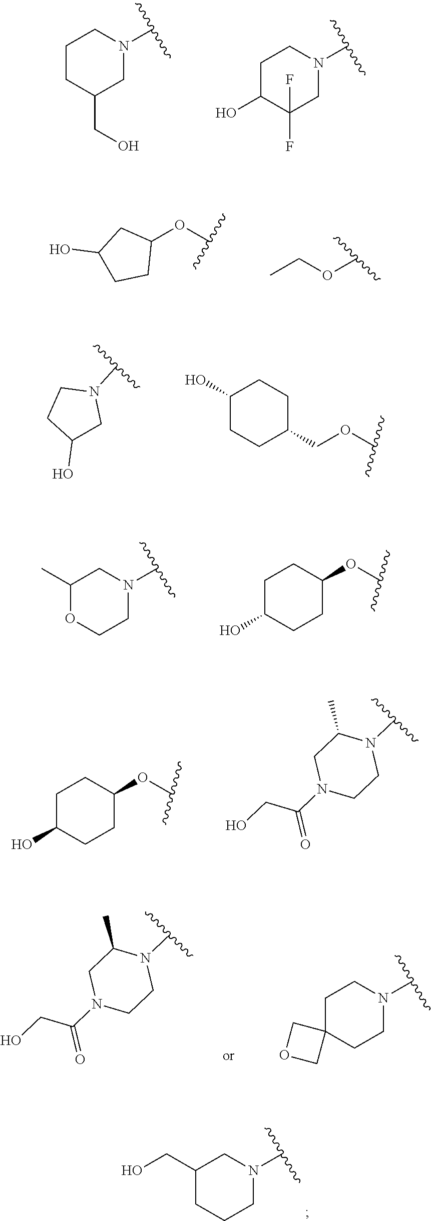

wherein the compound of formula (2a) comprises

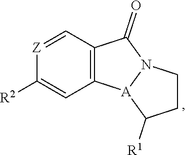

wherein G is N or CH; Z is CH or CF; R¹ is selected from the group consisting of

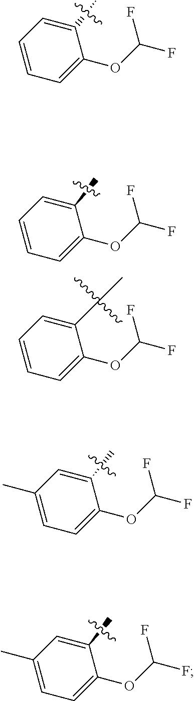

and R² is selected from the group consisting of

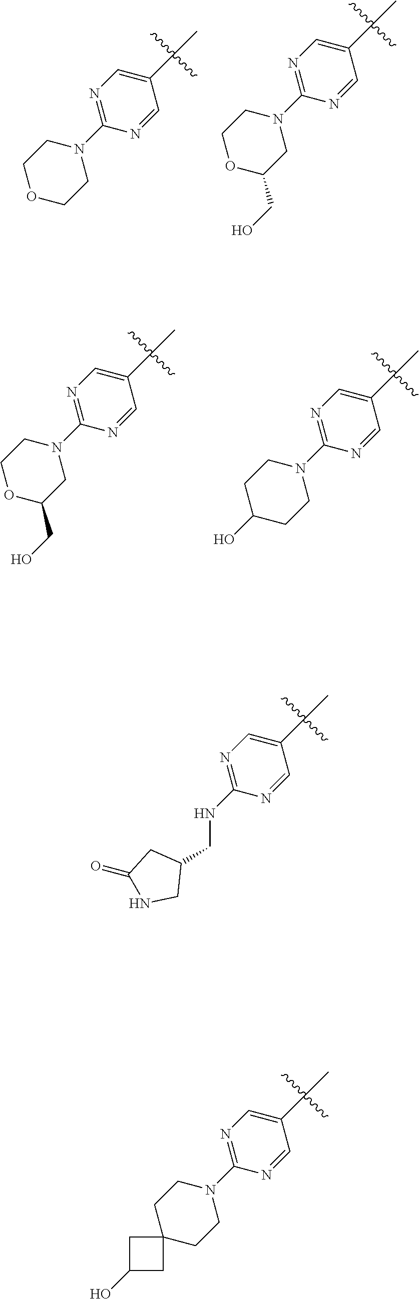

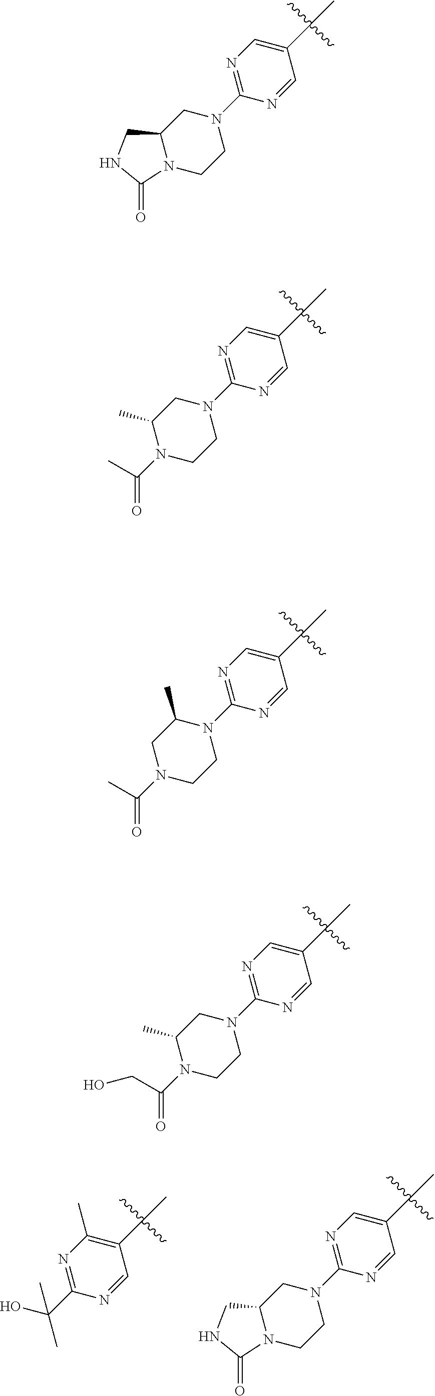 [SMILES: *c1cnc(N2CCN3C(=O)NC[C@H]3C2)nc1 ; *c1cnc(N2CCN(C(C)=O)[C@H](C)C2)nc1 ; *c1cnc(N2CCN(C(C)=O)C[C@H]2C)nc1 ; *c1cnc(N2CCN(C(=O)CO)[C@H](C)C2)nc1 ; *c1cnc(C(C)(C)O)nc1C ; *c1cnc(N2CCN3C(=O)NC[C@@H]3C2)nc1]

Embodiment 29 provides the compound of any one of Embodiments 3-25, wherein the AATM comprises one of the following:
a FcRn antagonist,

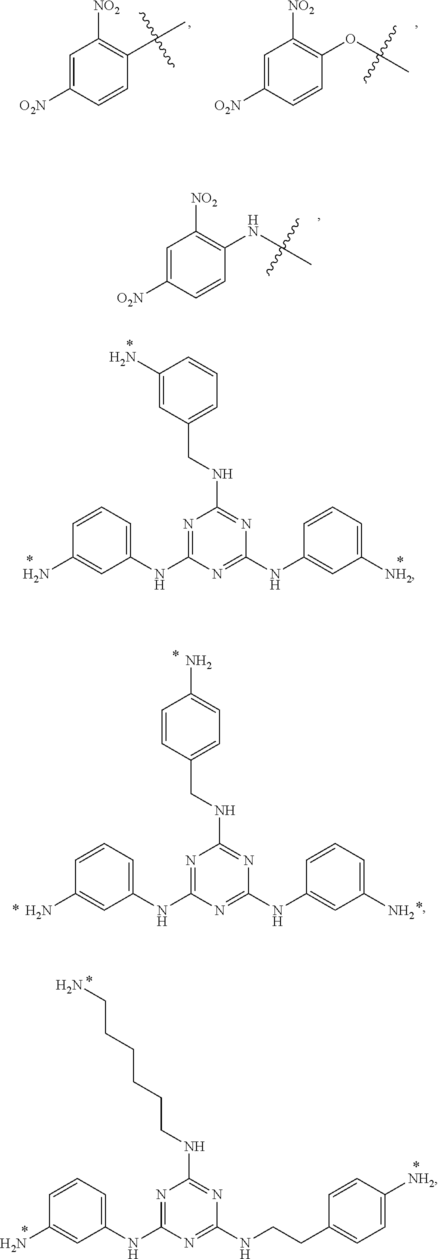 [SMILES: *c1ccc([N+](=O)[O-])cc1[N+](=O)[O-] ; *Oc1ccc([N+](=O)[O-])cc1[N+](=O)[O-] ; *Nc1ccc([N+](=O)[O-])cc1[N+](=O)[O-] ; Nc1cccc(CNc2nc(Nc3cccc(N)c3)nc(Nc3cccc(N)c3)n2)c1 ; Nc1ccc(CNc2nc(Nc3cccc(N)c3)nc(Nc3cccc(N)c3)n2)cc1 ; NCCCCCCNc1nc(NCCc2ccc(N)cc2)nc(Nc2cccc(N)c2)n1]

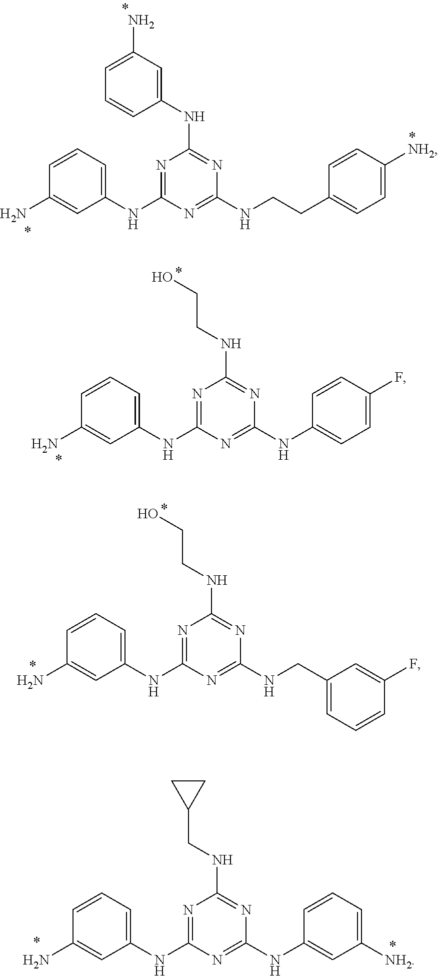 [SMILES: Nc1ccc(CCNc2nc(Nc3cccc(N)c3)nc(Nc3cccc(N)c3)n2)cc1 ; Nc1cccc(Nc2nc(NCCO)nc(Nc3ccc(F)cc3)n2)c1 ; Nc1cccc(Nc2nc(NCCO)nc(NCc3cccc(F)c3)n2)c1 ; Nc1cccc(Nc2nc(NCC3CC3)nc(Nc3cccc(N)c3)n2)c1]

cyclic peptide FcIII, or any reduced form thereof,
cyclic peptide FcIII-4C (amide), or any reduced form thereof,
a compound of formula (3a) or (3b):

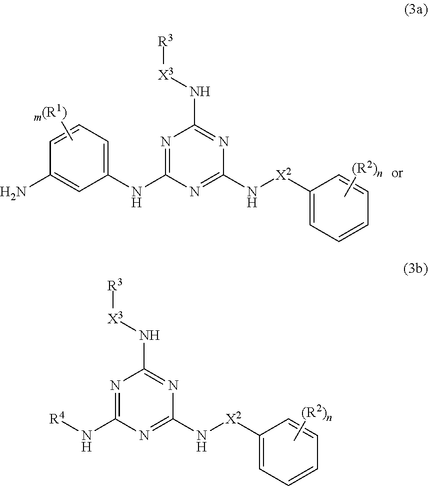

wherein:
each occurrence of R¹ is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
m is 0, 1, 2, 3, or 4;
X² is a bond, optionally substituted C₁-C₆ alkyl, or optionally substituted C₁-C₆ heteroalkyl:
each occurrence of R² is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
n is 0, 1, 2, 3, or 4;
X³ is a bond, optionally substituted C₁-C₆ alkyl, or optionally substituted C₁-C₆ heteroalkyl;
R³ is H, R, —OH, —NH₂, —NHR, —C(═O)OH, or —SH, wherein each occurrence of R is C₁-C₆ alkyl or C₃-C₈ cycloalkyl;
R⁴ is cycloalkyl, including polycyclic cycloalkyl, which is optionally substituted with 1-4 groups independently selected from the group consisting of F, Cl, Br, I, CN, NO₂, R, OR, C₁-C₆ haloalkyl, C₃-C₈ halocycloalkyl, C₁-C₆ haloalkoxy, C₃-C₈s halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
a compound of formula:

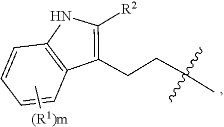

wherein:
each occurrence of R¹ is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
m is 0, 1, 2, 3, or 4;
each occurrence of R² is independently H, F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl.

Embodiment 30 provides the compound of Embodiment 29, wherein the FcRn antagonist comprises rozanolixizumab or efgartigimod.

Embodiment 31 provides a pharmaceutical composition comprising at least one pharmaceutically acceptable excipient at least one compound of any one of Embodiments 1-30.

Embodiment 32 provides the pharmaceutical composition of Embodiment 31, further comprising another therapeutically agent that treats, ameliorates, and/or prevents a disease or disorder.

Embodiment 33 provides a method of treating, ameliorating, and/or preventing a disease or disorder in a subject, the method comprising administering a therapeutically effective amount of at least one compound of any one of Embodiments 1-30 and/or at least one pharmaceutical composition of any one of Embodiments 31-32.

Embodiment 34 provides the method of Embodiment 33, wherein the disease or disorder comprises an autoimmune disease, cancer, or inflammation.

Embodiment 35 provides the method of Embodiment 34, wherein the autoimmune disease comprises Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (APIS), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymyositis, acute disseminated encephalomyelitis (ADEM), acute motor axonic neuropathy, anti-NMDA receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Meniere's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, or pyoderma gangrenosum.

Embodiment 36 provides the method of Embodiment 34, wherein the cancer comprises prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer, or lymphoma.

Embodiment 37 provides the method of Embodiment 34, wherein the inflammation comprises inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, or arteriosclerosis.

Embodiment 38 provides the method of any one of Embodiments 33-37, wherein the subject is further administered at least one additional therapeutic agent that treats, ameliorates, and/or prevents the disease or disorder.

Embodiment 39 provides the method of any one of Embodiments 33-38, wherein the subject is a mammal.

Embodiment 40 provides the method of any one of Embodiments 33-39, wherein the subject is a human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound comprising at least one formula selected from:
formula (I):
[Protein binder]_{k′}-[CON]ₕ-[Linker]ᵢ-[CON]_{h′}-[CRBM]_{j′} (I),
wherein:
the Protein binder is a molecule that binds to an extracellular protein;
the CRBM is a cellular receptor binding moiety that binds to at least one receptor on the surface of a degrading cell in a subject, whereby binding of (I) leads to endocytosis and degradation of the extracellular protein;
each CON is independently a bond or a group that covalently links a Protein binder to an CRBM, a Protein binder to a Linker, aid/or a Linker to a CRBM;
the Linker is a group having a valence of 1, 2, 3, 4, 5, 6, 7, 8, 9, 10, 11, 12, 13, 14, or 15;
k′ is 1, 2, 3, 4, 5, 6, 7, 8, 9, 10, 11, 12, 13, 14, or 15;
h is 0, 1, 2, 3, 4, 5, 6, 7, 8, 9, 10, 11, 12, 13, 14, or 15;
i is 0, 1, 2, 3, 4, 5, 6, 7, 8, 9, 10, 11, 12, 13, 14, or 15;
h′ is 0, 1, 2, 3, 4, 5, 6, 7, 8, 9, 10, 11, 12, 13, 14, or 15; and
j is 1, 2, 3, 4, 5, 6, 7, 8, 9, 10, 11, 12, 13, 14, or 15;
formula (II):
[TNF binder]_{k′}-[CON]ₕ-[Linker]ᵢ-[CON]_{h′}-[CRBM]_{j′} (II),
wherein:
the TNF binder is a molecule that binds to TNF;
the CRBM is a cellular receptor binding moiety that binds to at least one receptor on the surface of a degrading cell in a subject, whereby binding of (II) leads to endocytosis and degradation of TNF;
each CON is independently a bond or a group that covalently links a TNF binder to an CRBM, a TNF binder to a Linker, or a Linker to a CRBM;
the Linker is a group having a valence of 1, 2, 3, 4, 5, 6, 7, 8, 9, 10, 11, 12, 13, 14, or 15;
k′ is 1,2,3,4, 5,6,7, 8,9, 10, 11, 12, 13, 14, or 15:
h is 0, 1,2,3,4,5,6,7,8,9, 10, 11, 12, 13, 14, or 15;
i is 0, 1,2,3,4,5,6,7,8,9, 10, 11, 12, 13, 14, or 15:
h′ is 0, 1,2,3,4,5,6,7,8,9, 10, 11, 12, 13, 14, or 15; and
j is 1, 2, 3, 4, 5, 6, 7, 8, 9, 10, 11, 12, 13, 14, or 15:
formula (III):
[AATM]_{k′}-[CON]ₕ-[Linker]ᵢ-[CON]_{h′}-[CRBM]_{j′} (III),
wherein:
the AATM is a molecule that binds to an autoantibody:
the CRBM is a cellular receptor binding moiety that binds to at least one receptor on the surface of a degrading cell in a subject, whereby binding of (III) leads to endocytosis and degradation of the autoantibody:
each CON is independently a bond or a group that covalently links an AATM to an CRBM, an AATM to a Linker, or a Linker to a CRBM:
the Linker is a group having a valence of 1, 2, 3, 4, 5, 6, 7, 8, 9, 10, 11, 12, 13, 14, or 15;
k′ is 1,2,3,4, 5,6,7, 8,9, 10, 11, 12, 13, 14, or 15:
h is 0, 1,2,3,4,5,6,7,8,9, 10, 11, 12, 13, 14, or 15;
i is 0, 1,2,3,4,5,6,7,8,9, 10, 11, 12, 13, 14, or 15:
h′ is 0, 1,2,3,4,5,6,7,8,9, 10, 11, 12, 13, 14, or 15; and
j is 1, 2, 3, 4, 5, 6, 7, 8, 9, 10, 11, 12, 13, 14, or 15:
or a salt, geometric isomer, stereoisomer, or solvate thereof.

2-3. (canceled)

4. The compound of claim 1, wherein at least one of the following applies:
the valence of the Linker is 1, 2, or 3,
k′ is 1, 2, or 3;
j is 1, 2, or 3;
h is 1, 2, or 3;
h′ is 1, 2, or 3;
i is 1, 2, or 3;
at least one of h, h′, and i is at least 1;
k′, i′, h, h′, and i are each independently 1, 2, or 3;
k′ is 1, and j′ is 1, 2, or 3.

5-12. (canceled)

13. The compound of claim 1, which is one of the following:
[Protein binder][CON]₀₋₁-[Linker][CON]₀₋₁-[CRBM] (Ia),
[TNF binder][CON]₀₋₁-[Linker][CON]₀₋₁-[CRBM] (IIa); or
[AATM][CON]₀₋₁-[Linker][CON]₀₋₁-[CRBM] (IIa).

14-15. (canceled)

16. The compound of claim 1, wherein the degrading cell comprises a hepatocyte.

17. The compound of claim 1, wherein the CRBM is a folic acid (folate) receptor binder, mannose receptor binder, mannose-6-phosphate (M6P) receptor binder, low density lipoprotein receptor-related protein 1 (LRP1) receptor binder, low density lipoprotein receptor (LDLR) binder, FcγRI receptor binder, transferrin receptor binder, macrophage scavenger receptor binder, G-Protein coupled receptor binder, or asialoglycoprotein receptor (ASGPR) binder.

18. The compound of claim 1, wherein the CRBM is:
(a) a folic acid (folate) receptor binder comprising at least one of folic acid, methotrexate, premetrexed, or a biologically active fragment thereof;
(b) a mannose receptor binder comprising at least one of: 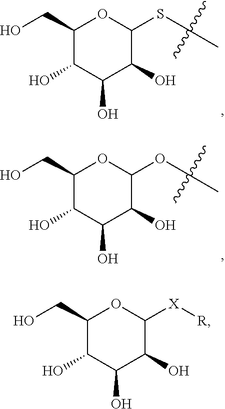
wherein: X is S or O, R is selected from the group consisting of: 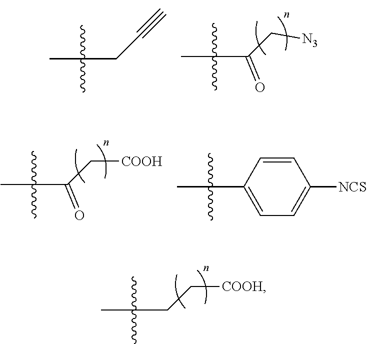
and each occurrence of ‘n’ is independently 1, 2, 3, 4, 5, 6, 7, 8, 9, 10, 11, 12, 13, 14, 15, 16, 17, 18, 19, or 20; and
a polymeric molecule selected from the group consisting of: 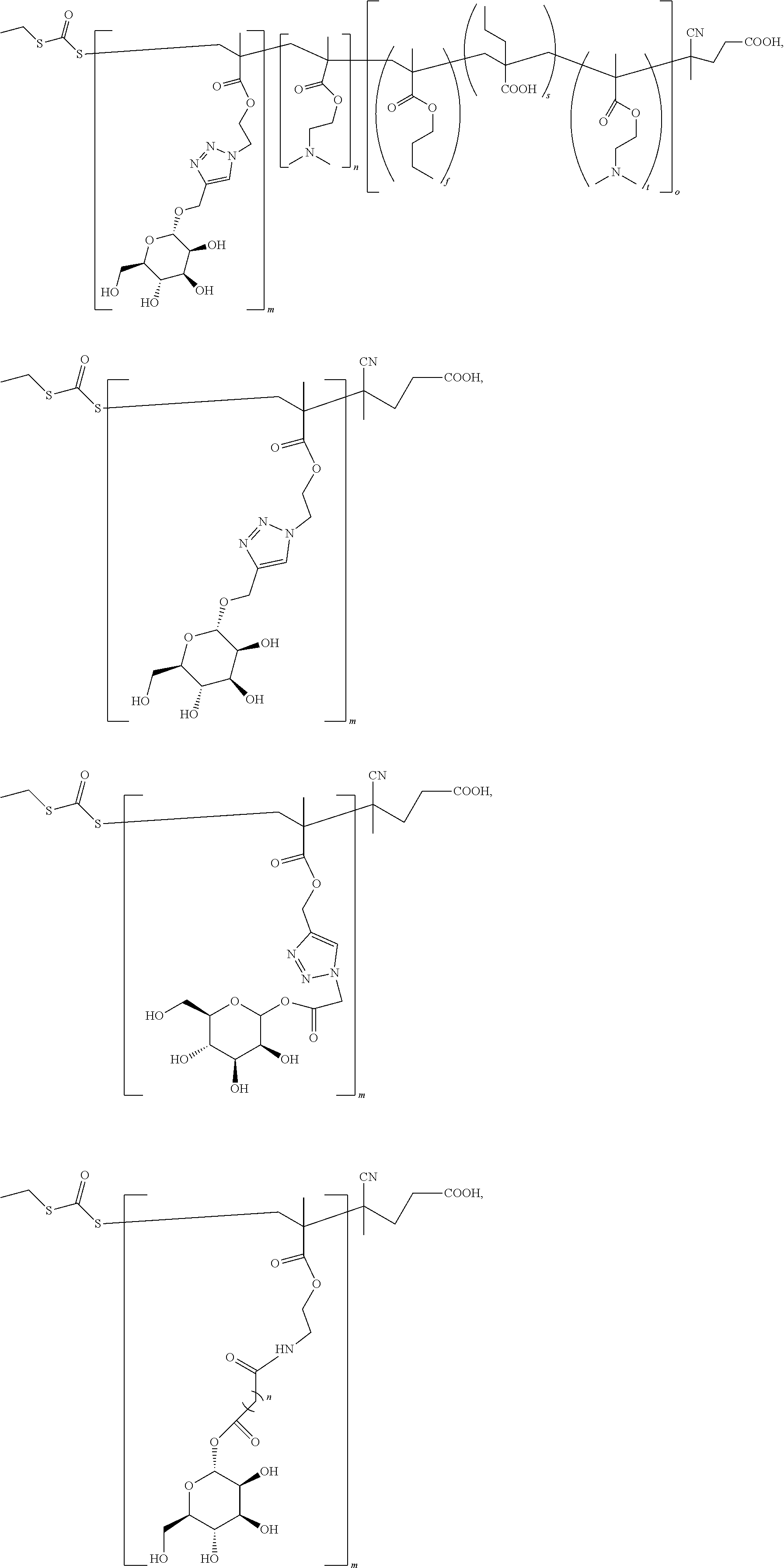 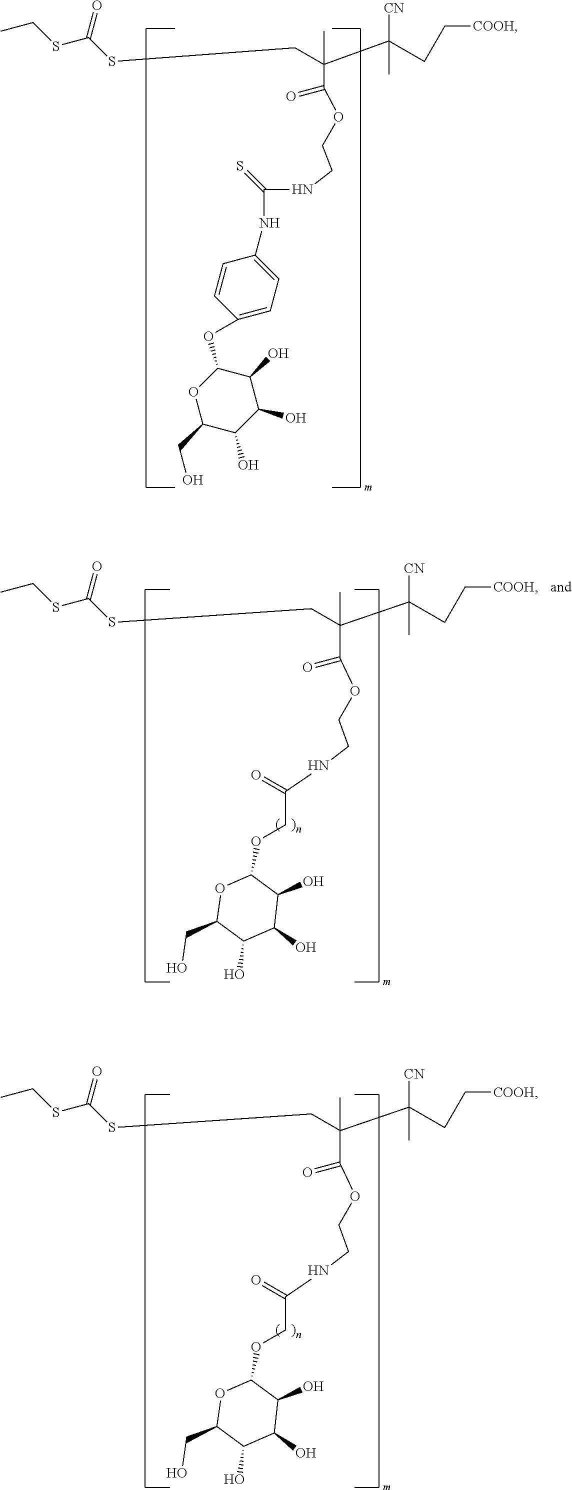
wherein
each occurrence of m is independently an integer from 1 to 100,
r, s, t, and o are each independently an integer from 0 to 100, and
the COOH in the polymeric molecule is derivatized with the Protein binder, the TNF binder, or the AATM;
(c) a mannose-6-phosphate (M6P) receptor binder comprising at least one of: 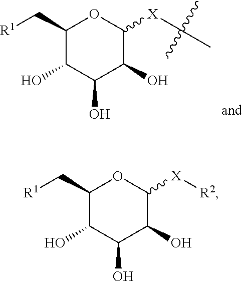
wherein X is O or S, R¹ is selected from the group consisting of: 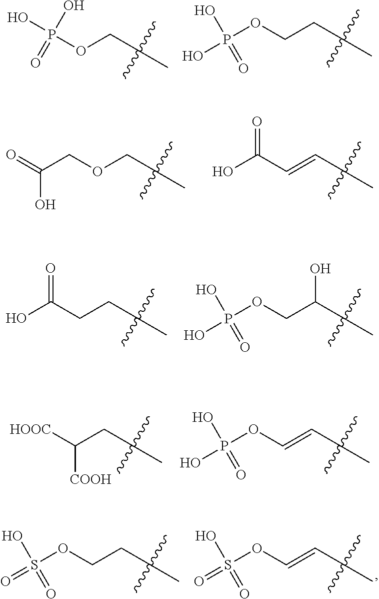
R² is selected from the group consisting of: 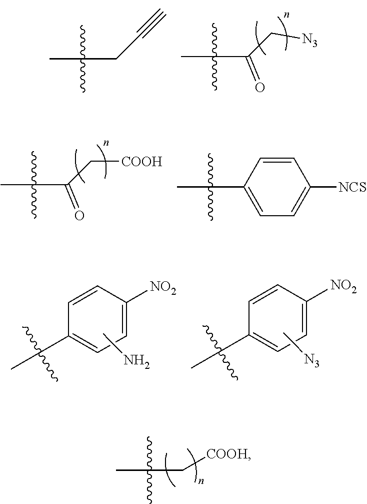
and each occurrence of ‘n’ is independently 1, 2, 3, 4, 5, 6, 7, 8, 9, 10, 11, 12, 13, 14, 15, 16, 17, 18, 19, or 20;
a polymeric molecule 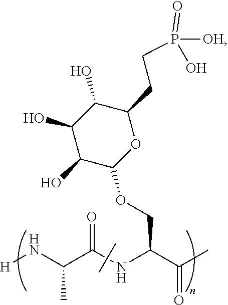
wherein n is an integer from 1 to 100;
a compound selected from: 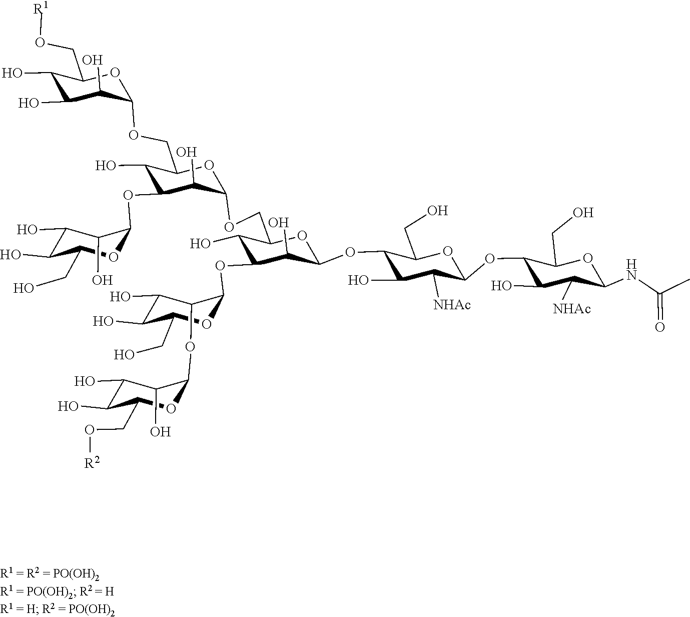 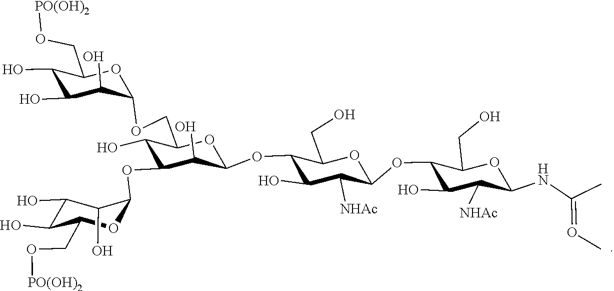
a compound selected from: 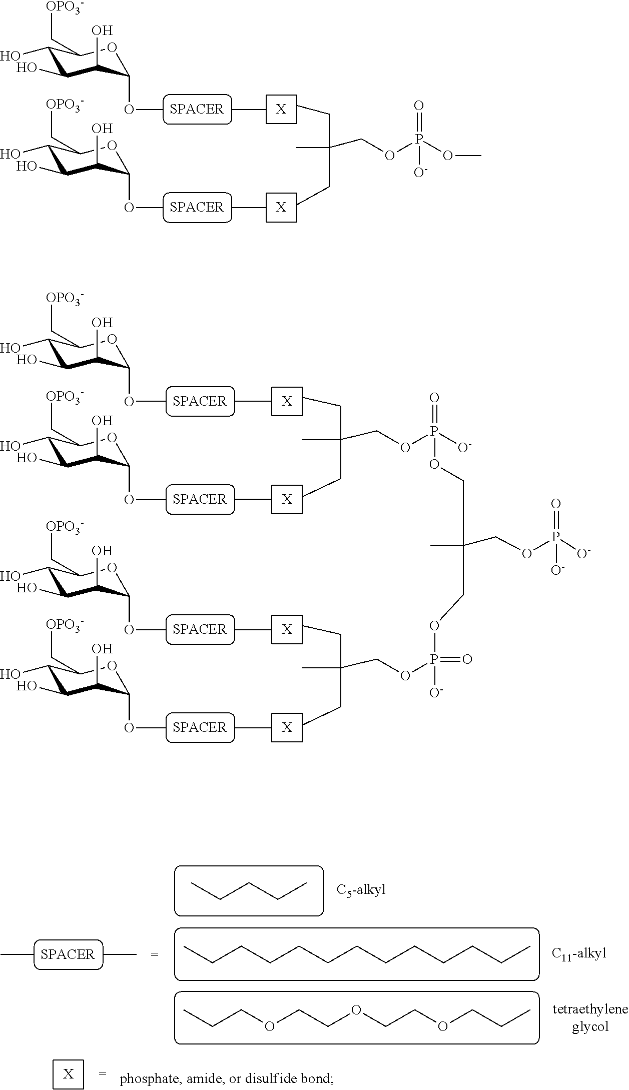
(d) a low density lipoprotein receptor-related protein 1 (LRP1) receptor binder comprising at least one amino acid sequence of SEQ ID NOs:1-9;
(e) a low density lipoprotein receptor (LDLR) binder comprising at least one amino acid sequence of SEQ ID NOs:10-35;
(f) a FcγRI receptor binder comprising at least one amino acid sequence of SEQ ID NOs:36-52;
(g) a transferrin receptor binder comprising at least one amino acid sequence of SEQ ID NOs:53-59 and 74;
(h) a macrophage scavenger receptor binder comprising at least one amino acid sequence of SEQ ID NOs:60-65;
(i) a G-protein coupled receptor binder comprising at least one of: 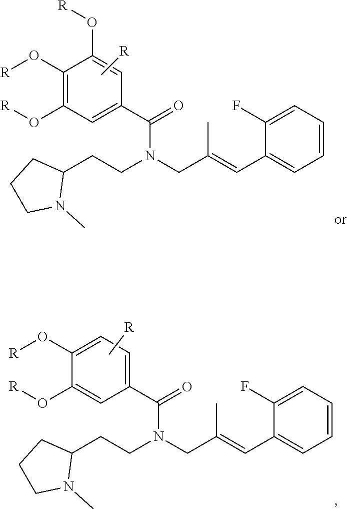
wherein each occurrence of R is independently H or C₁-C₆ alkyl;
(j) an asialoglycoprotein receptor (ASGPR) binder comprising: 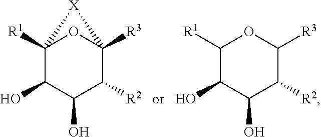
wherein:
X is a linker of 1-4 atoms in length and comprises O, S, N(R^{N1}), or C(R^{N1})(R^{N1}) groups, such that:
when X is a linker of 1 atom in length, X is O, S, N(R^{N1}), or C(R^{N1})(R^{N1}) when X is a linker of 2 atoms in length, no more than 1 atom of X is O, S, or N(R^{N1}),
when X is a linker of 3 or 4 atoms in length, no more than 2 atoms of X are independently O, S, or N(R^{N1});
wherein each occurrence of R^{N1} is independently H or C₁-C₃ alkyl optionally substituted with at least one of 1-3 independently selected halogens and 1-2 hydroxyl groups;
R¹ and R³ are each independently H, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}(vinyl), —O(CH₂)_{K}(vinyl), —(CH₂)_{K}(alkynyl), —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C₁-C₄ alkyl) optionally substituted with 1-3 independently selected halogens; or
R¹ and R³ are each independently Ph(CH₂)_{K}—, which is optionally substituted with: 1-3 independently selected halogens; C₁-C₄ alkyl optionally substituted with at least one of 1-3 independently selected halogens and 1-2 hydroxyl groups; or C₁-C₄ alkoxy optionally substituted with at least one of 1-3 independently selected halogens and 1-2 hydroxyl groups; or
R¹ and R³ are each independently a group of structure:
—O—(CH₂)_{K′}—CH(OH)—(CH₂)K′—R⁷,
wherein:
R⁷ is: C₁-C₄ alkoxy optionally substituted with at least one of 1-3 independently selected halogens and/er 1-2 hydroxy groups; —NR^{N3}R^{N4}; or
(CH₂)_{K′}—O—(CH₂)_{K}—CH₂—CH═CH₂;
K is 0, 1, 2, 3, or 4;
K′ is 1, 2, 3, or 4;
each occurrence of R^{N3} is independently H or C₁-C₃ alkyl optionally substituted with at least one of 1-3 independently selected halogens and 1-2 hydroxyl groups;
each occurrence of R^{N4} is independently H, C₁-C₃ alkyl optionally substituted with at least one of 1-3 independently selected halogens and 1-2 hydroxyl groups, or Ph-(CH₂)_{K}—; or
R¹ and R³ are each independently selected from the group consisting of:
(CH₂)_{K}OH, 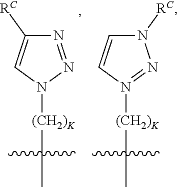
L¹-≡-L¹-(CH₂)_{K}—, and CYC-(CH₂)_{K}—, wherein CYC is selected from the group consisting of: 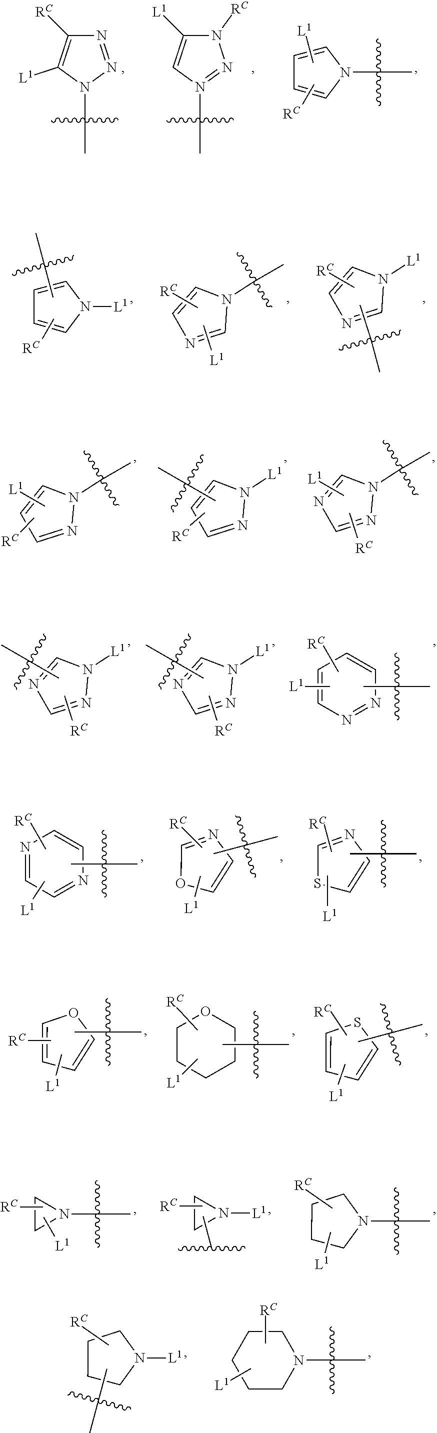 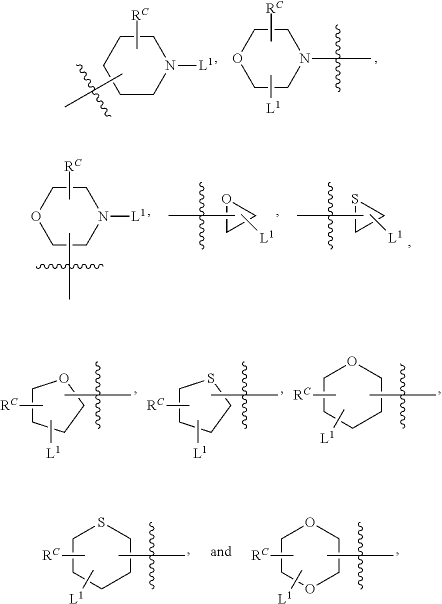
wherein:
the bond marked with [symbol] indicating the site on CYC whereto —(CH₂)_{K} is connected;
L¹ is a bond, -Linker, —CON-Linker, or —CON-Linker-CON;
R^{C} is absent, H, C₁-C₄ alkyl optionally substituted with at least one of 1-3 optionally substituted halogens and 1-2 hydroxyl groups, or a group of structure: 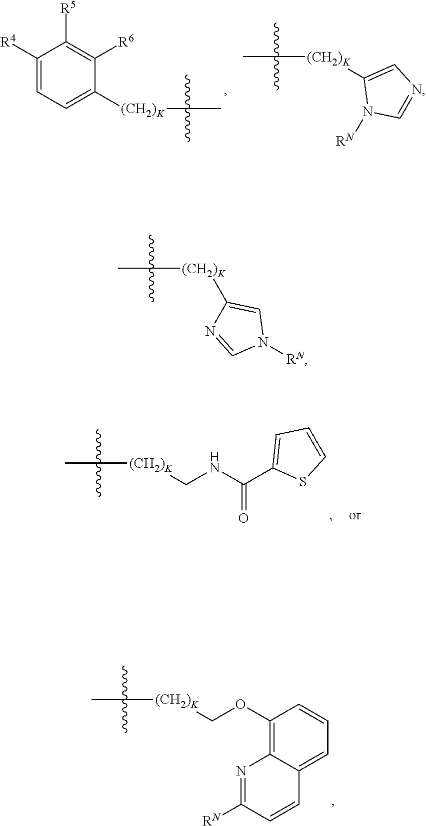
wherein:
R⁴, R⁵, and R⁶ are each independently H, F, Cl, Br, I, CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with 1-3 independently selected halogens, C₁-C₃-alkoxy optionally substituted with 1-3 independently selected halogens, —(CH₂)_{K}COOH, —(CH₂)_{K}C(═O)O—(C₁-C₄ alkyl) optionally substituted with 1-3 independently selected halogens, O—C(═O)—(C₁-C₄ alkyl) optionally substituted with 1-3 independently selected halogens, or —C(═O)—(C₁-C₄ alkyl) optionally substituted with 1-3 independently selected halogens;
each occurrence of R^{N} is independently H or C₁-C₃ alkyl optionally substituted with at least one of 1-3 independently selected halogens and 1-2 hydroxyl groups;
each occurrence of R^{N2} is independently H or C₁-C₃ alkyl optionally substituted with at least one of 1-3 independently selected halogens and 1-2 hydroxyl groups;
or
R¹ and R³ are each independently (C₃-C₈ saturated carbocyclic)-(CH₂)-, wherein the carbocyclic is further substituted with -L¹ and —R^{C};
R² is —(CH₂)_{K}—N(R^{N1})—C(═O)R^{AM}, wherein:
R^{AM} is H, C₁-C₄ alkyl optionally substituted with at least one of 1-3 independently selected halogens and 1-2 hydroxyl groups, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, —C(═O)(C1-C₄ alkyl) optionally substituted with 1-3 independently selected halogens, or —(CH₂)_{K}—NR^{N3}R^{N4}; or
R² is 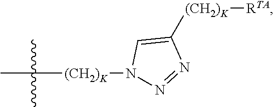
wherein:
R^{TA} is H, CN, NR^{N1}R^{N2}, —(CH₂)_{K}OH, —(CH₂)_{K}O(C₁-C₄ alkyl) optionally substituted with 1-3 independently selected halogens, C₁-C₄ alkyl optionally substituted with 1-3 independently selected halogens, —(CH₂)_{K}COOH, —(CH₂)_{K}C(═O)O(C₁-C₄ alkyl) optionally substituted with 1-3 independently selected halogens, —OC(═O)(C₁-C₄ alkyl) optionally substituted with 1-3 independently selected halogens, or —C(═O)(C1-C₄ alkyl) optionally substituted with 1-3 independently selected halogens, or
R^{TA} is C₃-C₁₀ aryl or a 3- to 10-membered heteroaryl group containing 1-5 non-carbon ring atoms, each of the aryl or heteroaryl groups being optionally substituted with 1-3 groups independently selected from CN, NR^{N1}R^{N2}, —(CH₂)_{K}OH, —(CH₂)_{K}O(C₁-C₄ alkyl) optionally substituted with 1-3 independently selected halogens, C₁-C₃ alkyl optionally substituted with at least one of 1-3 independently selected halogens and 1-2 hydroxyl groups, -(C₁-C₃-alkoxy) optionally substituted from 1-3 independently selected halogens, —(CH₂)_{K}COOH, —(CH₂)_{K}C(═O)O—(C₁-C₄ alkyl) optionally substituted with 1-3 independently selected halogens, —OC(═O)(C₁-C₄ alkyl) optionally substituted with 1-3 independently selected halogens, or —(CH₂)_{K}C(═O)—(C₁-C₄ alkyl) optionally substituted with 1-3 independently selected halogens, or
R^{TA} is 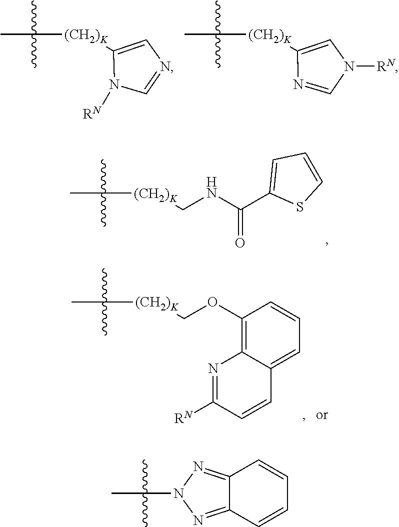
optionally substituted with 1-3 C₁-C₃ alkyl groups optionally substituted with 1-3 independently selected halogens, or
R^{TA} is 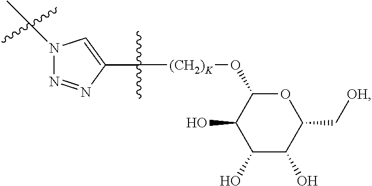
wherein each —(CH₂)_{K} group is optionally substituted with 1-4 C₁-C₃ alkyl groups optionally substituted with 1-3 fluoro groups or 1-2 hydroxyl groups.

19. The compound of claim 18, wherein:
the X in ASGPRBM is —O—C(R^{N1})(R^{N1})—, —C(R^{N1})(R^{N1})—O—, —S—C(R^{N1})(R^{N1})—, —C(R^{N1})(R^{N1})—S—, —N(R^{N1})—C(R^{N1})(R^{N1})—, —C(R^{N1})(R^{N1})—N(R^{N1})—, or —C(R^{N1})(R^{N1})—C(R^{N1})(R^{N1})—, when X is 2 atoms in length;
the X in ASGPRBM is —O—C(R^{N1})(R^{N1})—C(R^{N1})(R^{N1})—, —C(R^{N1})(R^{N1})—O—C(R^{N1})(R^{N1})—, —O—C(R^{N1})(R^{N1})—O—, —O—C(R^{N1})(R^{N1})—S—, —O—C(R^{N1})(R^{N1})—N(R^{N1})—, —S—C(R^{N1})(R^{N1})—C(R^{N1})(R^{N1})—, —C(R^{N1})(R^{N1})—S—C(R^{N1})(R^{N1})—, —C(R^{N1})(R^{N1})—C(R^{N1})(R^{N1})S—S—C(R^{N1})(R^{N1})—S—, —S—C(R^{N1})(R^{N1})—O—, —S—C(R^{N1})(R^{N1})—N(R^{N1})—, —N(R^{N1})—C(R^{N1})(R^{N1})—C(R^{N1})(R^{N1})—, —C(R^{N1})(R^{N1})—N(R^{N1})—C(R^{N1})(R^{N1})—, —C(R^{N1})(R^{N1})—C(R^{N1})(R^{N1})—N(R^{N1})—N(R^{N1})—C(R^{N1})(R^{N1})—N(R^{N1})—, or —C(R^{N1})(R^{N1})—C(R^{N1})(R^{N1})— C(R^{N1})(R^{N1}), when X is 3 atoms in length; or
the X in ASGPRBM is —O—C(R^{N1})(R^{N1})—C(R^{N1})(R^{N1})—C(R^{N1})(R^{N1})—, —C(R^{N1})(R^{N1})—O—C(R^{N1})(R^{N1})—C(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})—, —C(R^{N1})(R^{N1})—S—C(R^{N1})(R^{N1})—C(R^{N1})(R^{N1})—, —C(R^{N1})(R^{N1})—C(R^{N1})(R^{N1})—S—C(R^{N1})(R^{N1})—, —S—C(R^{N1})(R^{N1})—S—C(R^{N1})(R^{N1})—, —N(R^{N1})—C(R^{N1})(R^{N1})—C(R^{N1})(R^{N1})— C(R^{N1})(R^{N1})—, or —C(R^{N1})(R^{N1})—N(R^{N1})—C(R^{N1})(R^{N1})—C(R^{N1})(R^{N1})—, when X is 4 atoms in length.

20. The compound of claim 18, wherein X is OCH₂ and R^{N1} is H, or wherein X is CH₂O and R^{N1} is H.

21. The compound of claim 18, wherein the ASGPRBM comprises the structure:

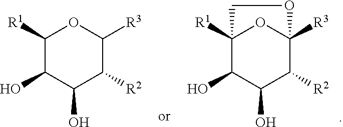

22. The compound of claim 18, wherein the ASGPRBM group comprises: 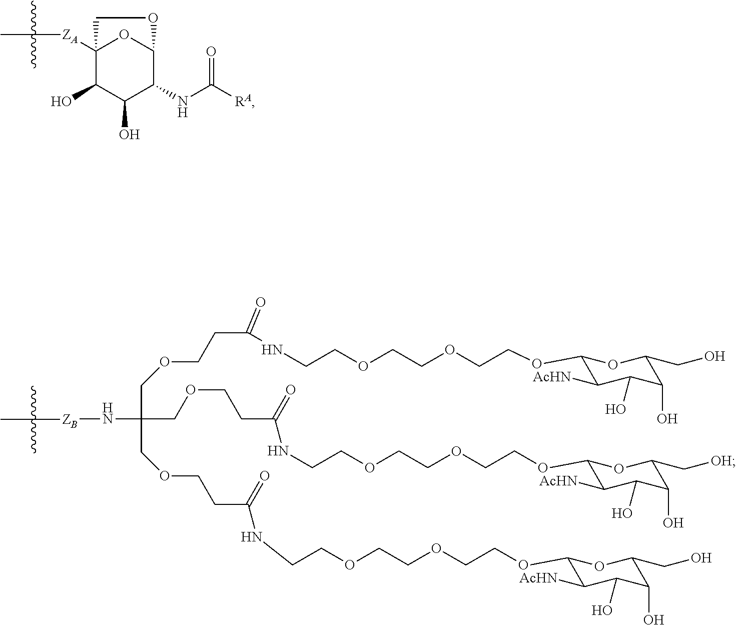
wherein:
R^{A} is C₁-C₃ alkyl optionally substituted with 1-5 independently selected halogens;
Z_{A} is —(CH₂)_{IM}-, —O—(CH₂)_{IM}-, —S—(CH₂)_{IM}-, —NM-(CH₂)_{IM}-, —C(═O)—(CH₂)_{IM}-, a PEG group containing from 1 to 8 ethylene glycol residues, or —C(O)(CH₂)_{IM}NR^{M}-;
Z_{B} is absent, —(CH₂)_{IM}-, —C(═O)—(CH₂)_{IM}-, or —C(═O)(CH₂)_{IM}—NR^{M}—;
R^{M} is H or C₁-C₃ alkyl optionally substituted with 1-2 hydroxyl groups; and
each occurrence of IM is independently 0, 1, 2, 3, 4, 5, or 6;
3 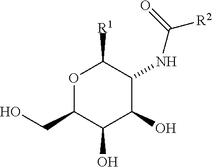
4

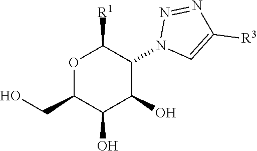

| | R¹ | # | R² |
|---|---|---|---|
| a | Me | 1 | Me |
| b | 4-OMePh | 2 | n-C₃H₇ |
| | | 3 | i-Pr |
| | | 4 | t-Bu |
| | | 5 | CCl₃ |
| | | 6 | CF₃ |
| | | 7 | Ot-Bu |
| | | 8 | CH₂CO₂H |
| | | 9 | CH₂NH₂ |
| | | 10 | CH₂CF₃ |
| | | 11 | 2-furyl |
| | | 12 | Ph |
| | | 13 | 4-OMePh |
| | | 14 | 3-OMePh |
| | | 15 | 4-CNPh |
| | | 16 | 5-pyridyl |
| | | 17 | 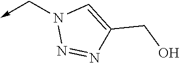 |
| | | # | R³ |
| | | 1 | Ph |
| | | 2 | 4-OMePh |
| | | 3 | 3-OMePh |
| | | 4 | 3-NH₂Ph |
| | | 5 | 4-NMe₂Ph |
| | | 6 | 2-pyridyl |
| | | 7 | 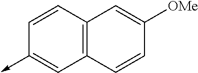 |
| | | 8 | 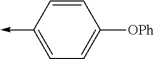 |
| | | 9 | 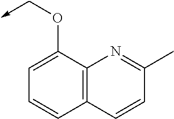 |
| | | 10 | 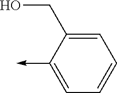 |
| | | 11 | 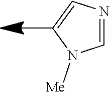 |
| | | 12 | 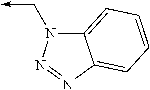 |
| | | 13 | 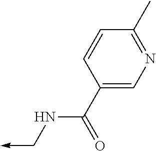 |
| | | 14 | 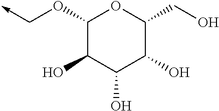 |
| | | 15 | CH₂CH |
| | | 16 | CH₂CH₂ |
| | | 17 | CH₂NHMe |
| | | 18 | CH₂NMe₂ |
| | | 19 | CO₂H |
| | | 20 | CH₂NHCOPh |
| | | 21 | CH₂NHCOMe |

6a-j

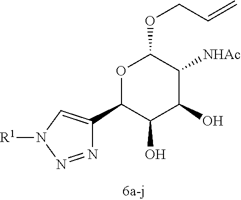

| | R¹ |
|---|---|
| a | Me |
| b | 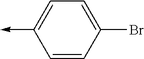 |
| c | 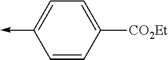 |
| d | 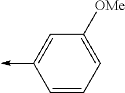 |
| e | 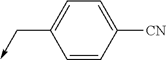 |
| f | 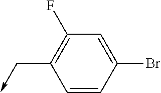 |
| g | 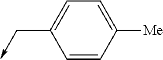 |
| h | 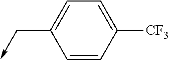 |
| i | 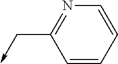 |
| j | 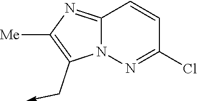 |

8a-j

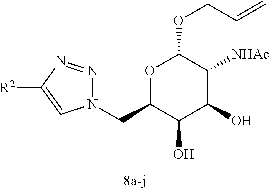

| | R² |
|---|---|
| a | CH₂OH |
| b | 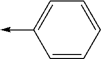 |
| c | 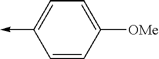 |
| d | 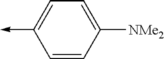 |
| e | 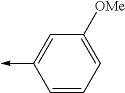 |
| f | 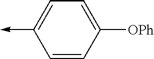 |
| g | 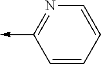 |
| h | 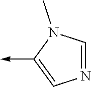 |
| i | 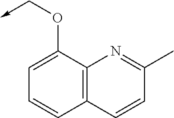 |
| j | 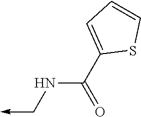 |

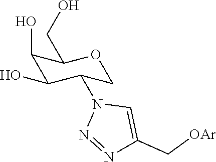

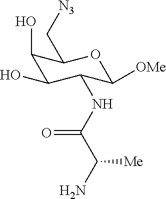

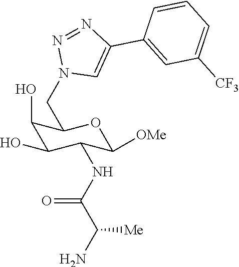

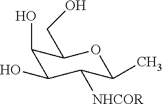

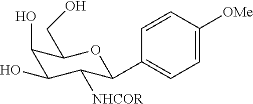

wherein
R=CH₃, CF₃, or CH₂CF₃;

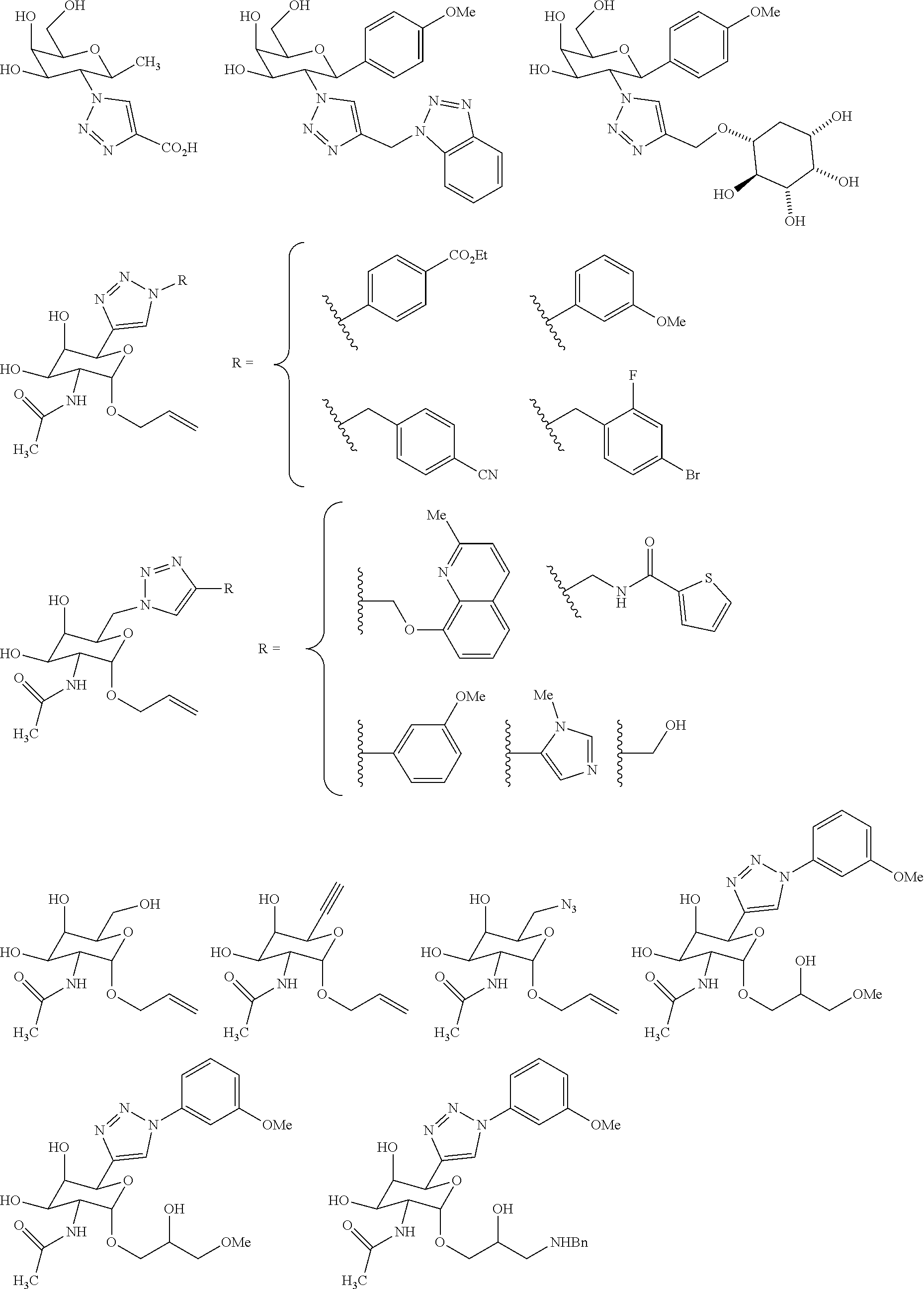

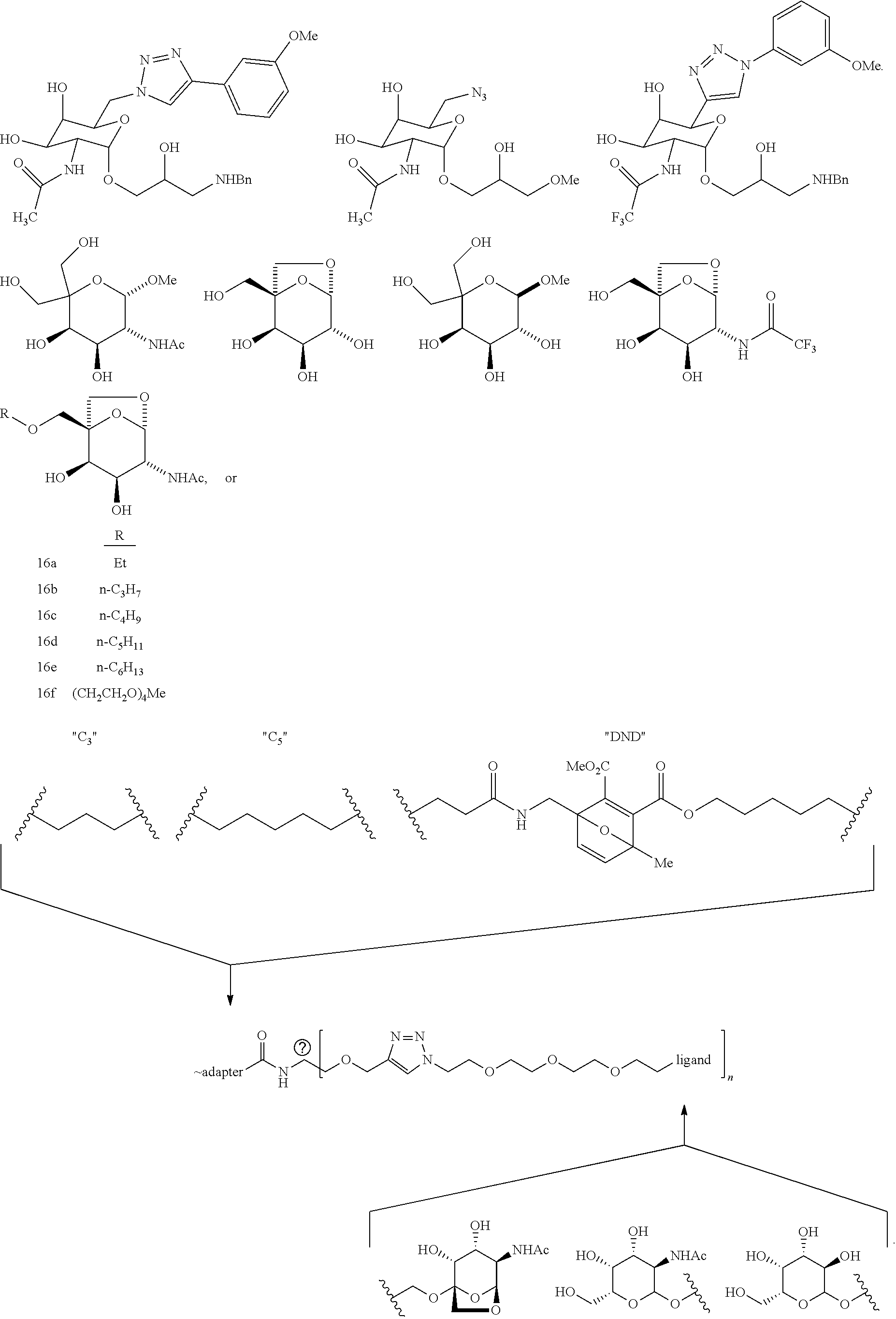

23. The compound of claim 1, wherein the Linker is a polyethylene glycol containing linker having 1-12 ethylene glycol residues.

24. The compound of claim 1, wherein the Linker comprises the structure:
—CH₂CH₂(OCH₂CH₂)ₘOCH₂—, —(CH₂)ₘCH₂—, —[N(R^{a})—CH(R^{b})(C═O)]ₘ-,
or a polypropylene glycol or polypropylene-co-polyethylene glycol group containing 1-100 alkylene glycol units;
wherein each R^{a} is independently H, C₁-C₃ alkyl, or C₁-C₆ alkanol, or combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine,—CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, and —(CH₂)phenol; and
wherein m is an integer ranging from 1 to 15; or
wherein the Linker comprises the structure
-[N(R′—(CH₂)₁₋₁₅—C(═O)]-,
wherein R′ is H or a C₁-C₃ alkyl optionally substituted with 1-2 hydroxyl groups, and m is an integer ranging from 1 to 100; or
wherein the Linker comprises the structure:
—Z-D-Z′-,
wherein:
Z and Z′ are each independently a bond, —(CH₂)ᵢ—O—, —(CH₂)ᵢ—S—, —(CH₂)ᵢ—N(R)-, 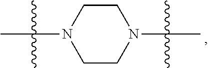 [SMILES: *N1CCN(*)CC1]
—(CH₂)ᵢ—C(R²)═C(R²)-(cis or trans), —(CH₂)ᵢ-≡-, or -Y—C(═O)—Y—;
each R is independently H, C₁-C₃ alkyl, or C₁-C₆ alkanol;
each R² is independently H or C₁-C₃ alkyl;
each Y is independently a bond, O, S, or N(R);
each i is independently 0 to 100;
D is a bond, —(CH₂)ᵢ-Y—C(═O)—Y—(CH₂)ᵢ-, —(CH₂)_{m′}-, or -[(CH₂)ₙ-X₁)]ⱼ-, with the proviso that Z, Z′, and D are not each simultaneously bonds;
X¹ is O, S, or N(R);
j is an integer ranging from 1 to 100;
m′ is an integer ranging from 1 to 100;
n is an integer ranging from 1 to 100; or
wherein the Linker comprises the structure:
—CH₂—(OCH₂CH₂)ₙ—CH₂—, —(CH₂CH₂O)_{n′}CH₂CH₂—, or —(CH₂CH₂CH₂O)ₙ—,
wherein each n and n′ is independently an integer ranging from 1 to 25; or
wherein the Linker comprises a structure:
-PEG-CON-PEG-
wherein each PEG is independently a polyethylene glycol group containing from 1-12 ethylene glycol residues and CON is a triazole group.

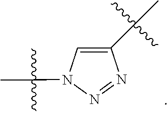 [SMILES: *c1cn(*)nn1]

25. The compound of claim 1,
wherein the CON comprises a structure: 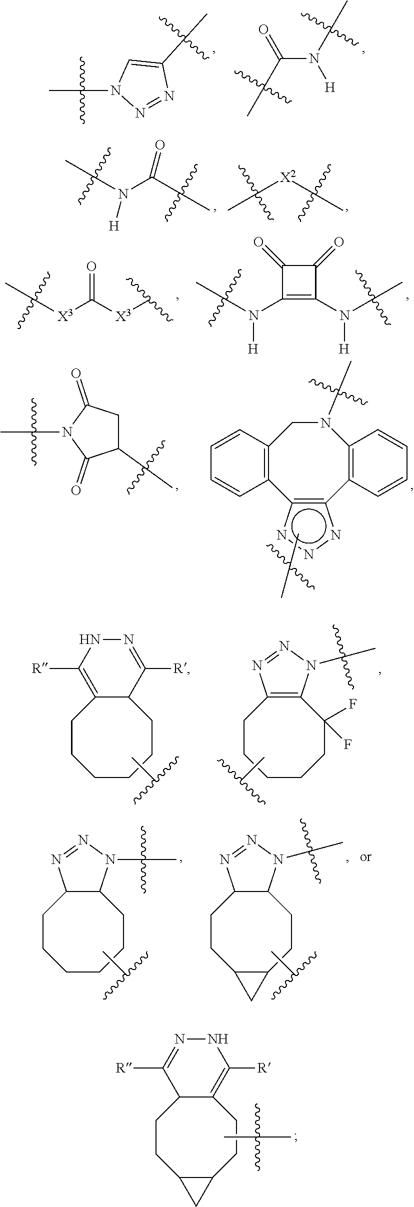
wherein R′ and R″ are each independently H, methyl, or a bond; or
wherein the CON comprises a structure:
—C(═O)—N(R¹)—(CH₂)_{n″}—N(R¹)C(═O)—,
—N(R¹)—C(═O)(CH₂)_{n″}—C(═O)N(R¹)-, or
—N(R¹)—C(═O)(CH₂)_{n″}—N(R¹)C(═O)—;
wherein each R¹ is independently H or C₁-C₃ alkyl, and n″ is independently 0, 1, 2, 3, 4, 5, 6, 7, or 8; or
wherein the CON comprises a structure: 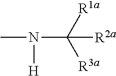
wherein:
R^{1a}, R^{2a} and R^{3a} are each independently H, —(CH₂)_{M1}-, —(CH₂)_{M2}C(═O)_{M3}(NR⁴)_{M3}—(CH₂)_{M2}-, —(CH₂)_{M2}(NR⁴)_{M3}C(O)_{M3}—(CH₂)_{M2}-, or —(CH₂)_{M2}O—(CH₂)_{M1}—C(O)NR⁴—, with the proviso that Ria, R^{2a} and R^{3a} are not simultaneously H;
each M1 is independently 1, 2, 3, or 4;
each M2 is independently 0, 1, 2, 3, or 4;
each M3 is independently 0 or 1; and
each R⁴ is independently H, C₁-C₃ alkyl, C₁-C₆ alkanol, or —C(═O)(C1-C₃ alkyl), with the proviso that M2, and M3 within the same R^{1a}, R^{2a} and R^{3a} cannot all be simultaneously 0;
or
wherein the CON comprises a structure:

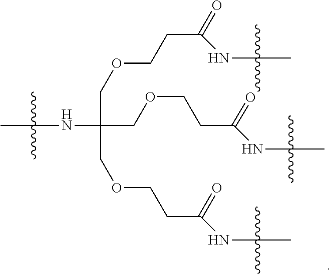 [SMILES: *NC(=O)CCOCC(COCCC(=O)N*)(COCCC(=O)N*)N*]

26. The compound of claim 1,
wherein the Protein binder that binds to CD40L comprises: 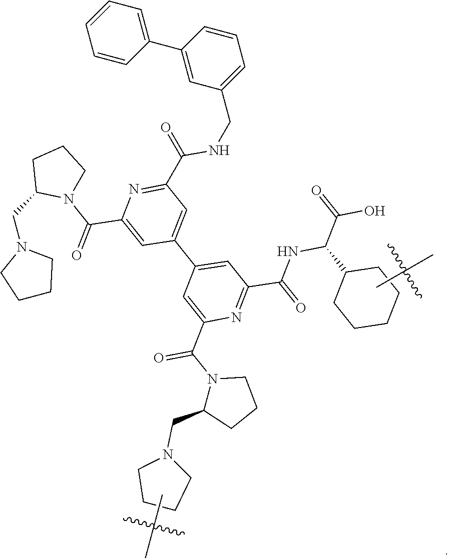
wherein the Protein binder that binds to PCSK9 comprises: 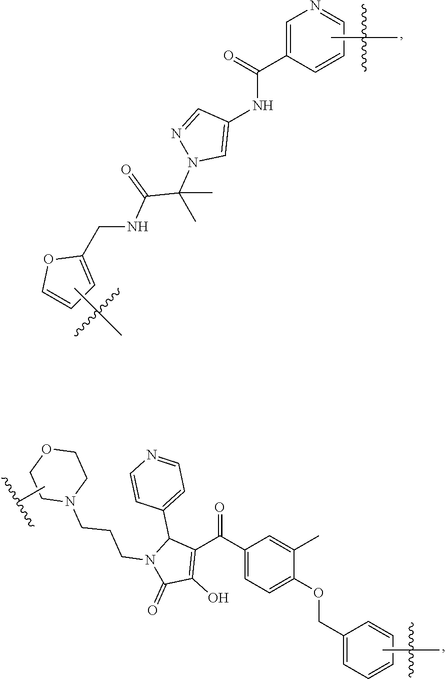 [symbol]
NH-TVFTSWEEYLDWV-X (SEQ ID NO:66), wherein X=OH or NH2; 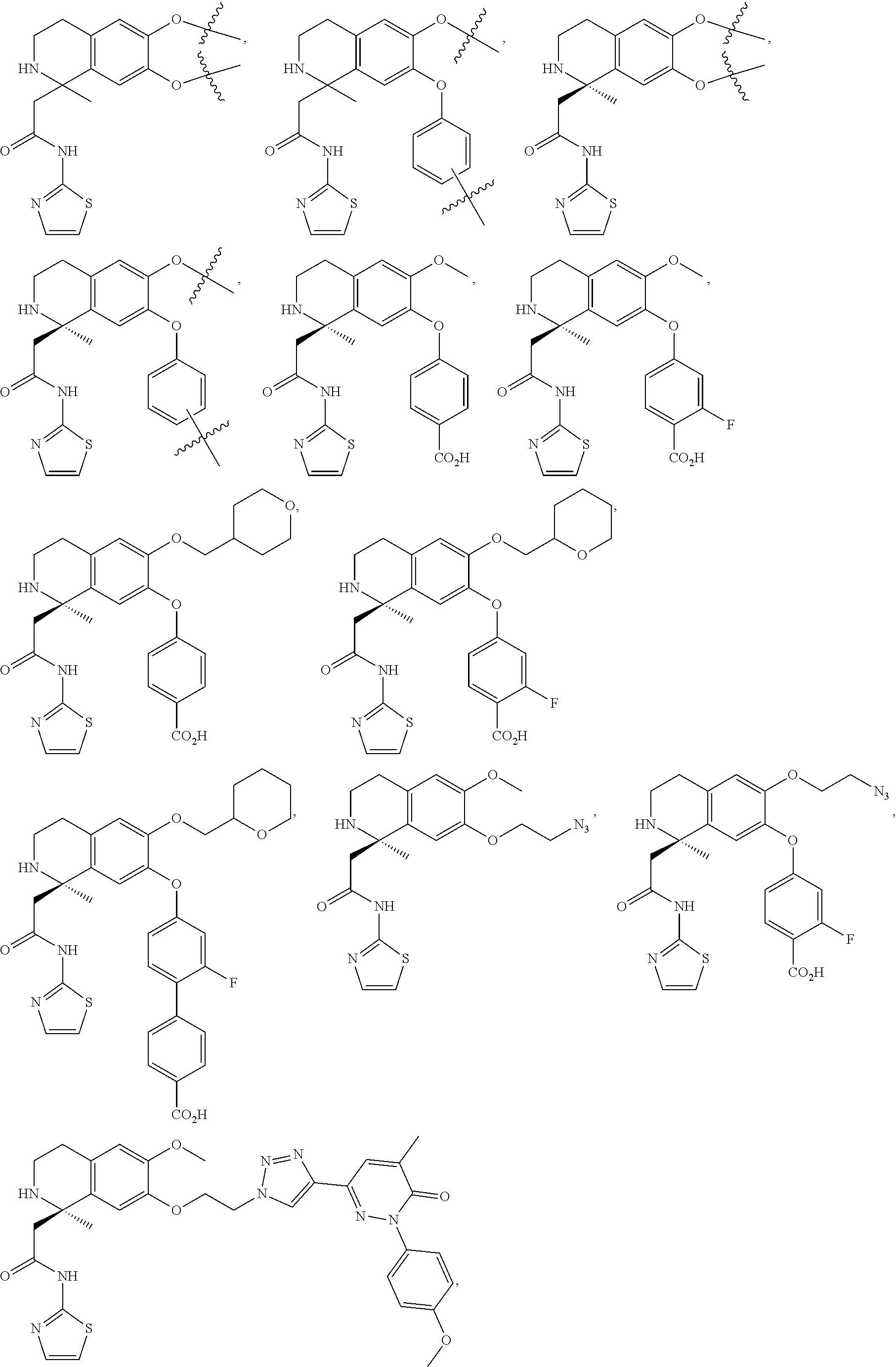 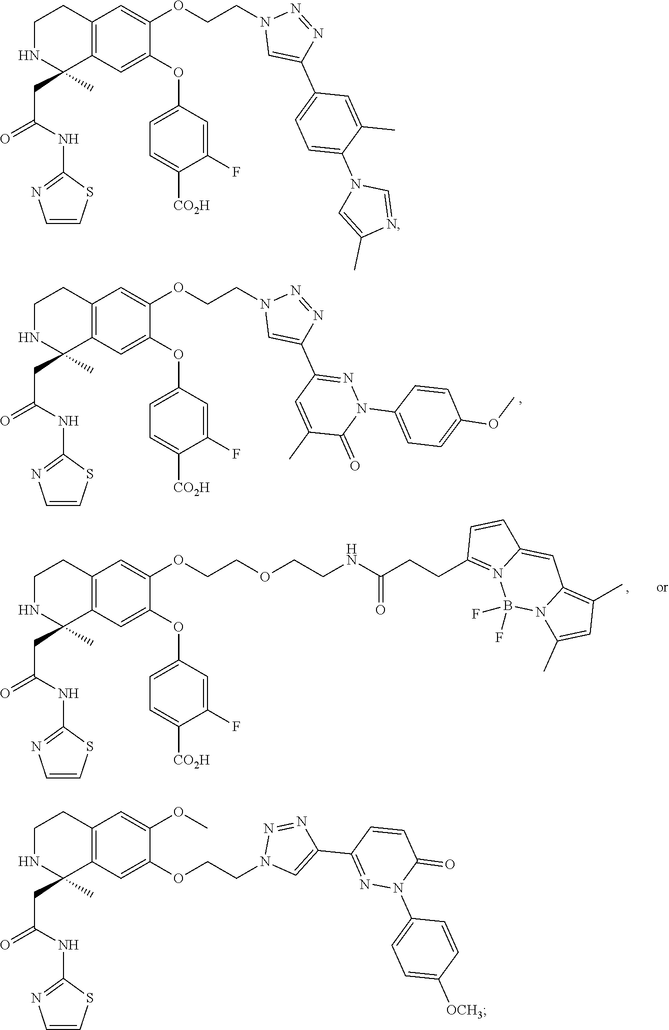
wherein the Protein binder that binds to VEGF comprises: [symbol]
NH-VEPNCDIHVMWEWECFERL-X (SEQ ID NO:67), wherein X=OH or NH2, 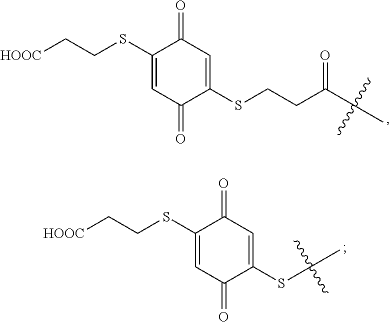 [SMILES: *C(=O)CCSC1=CC(=O)C(SCCC(=O)O)=CC1=O ; *SC1=CC(=O)C(SCCC(=O)O)=CC1=O]
wherein the Protein binder that binds to TGF-beta comprises: [symbol]
NH-KRFKQDGGC-X (SEQ ID NO:68), wherein X=OH or NH2; 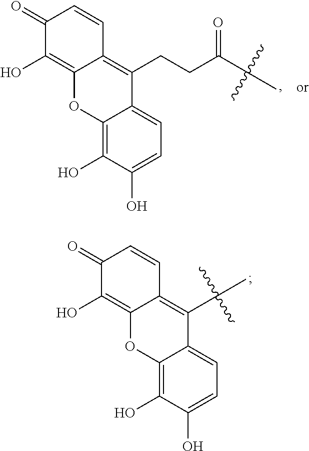
wherein the Protein binder that binds to TSP-1 comprises: [symbol]
NH-RGQILSKLRL-X (SEQ ID NO:69), wherein X=OH or NH₂;
wherein the Protein binder that binds to soluble uPAR comprises: 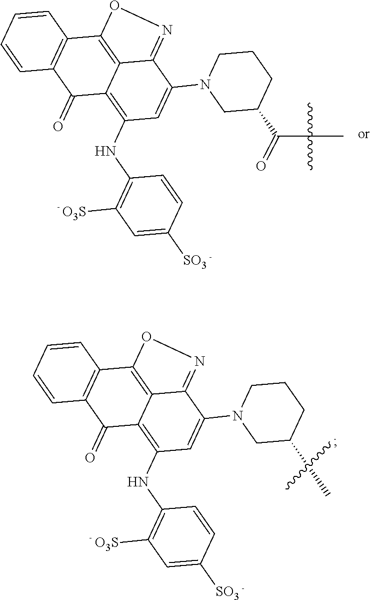
wherein the Protein binder that binds to soluble PSMA comprises: 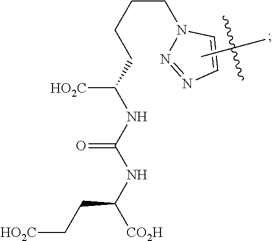
wherein the Protein binder that binds to IL-2 comprises: 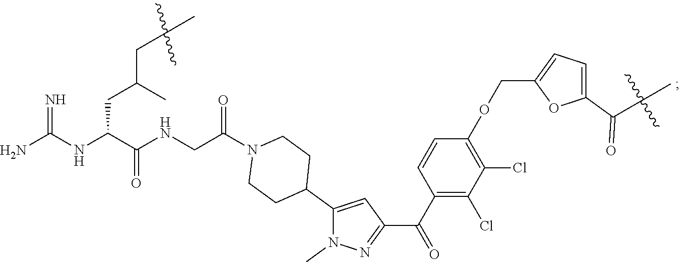 [SMILES: *CC(C)C[C@@H](NC(=N)N)C(=O)NCC(=O)N1CCC(c2cc(C(=O)c3ccc(OCc4ccc(C(*)=O)o4)c(Cl)c3Cl)nn2C)CC1]
wherein the Protein binder that binds to GP120 comprises: 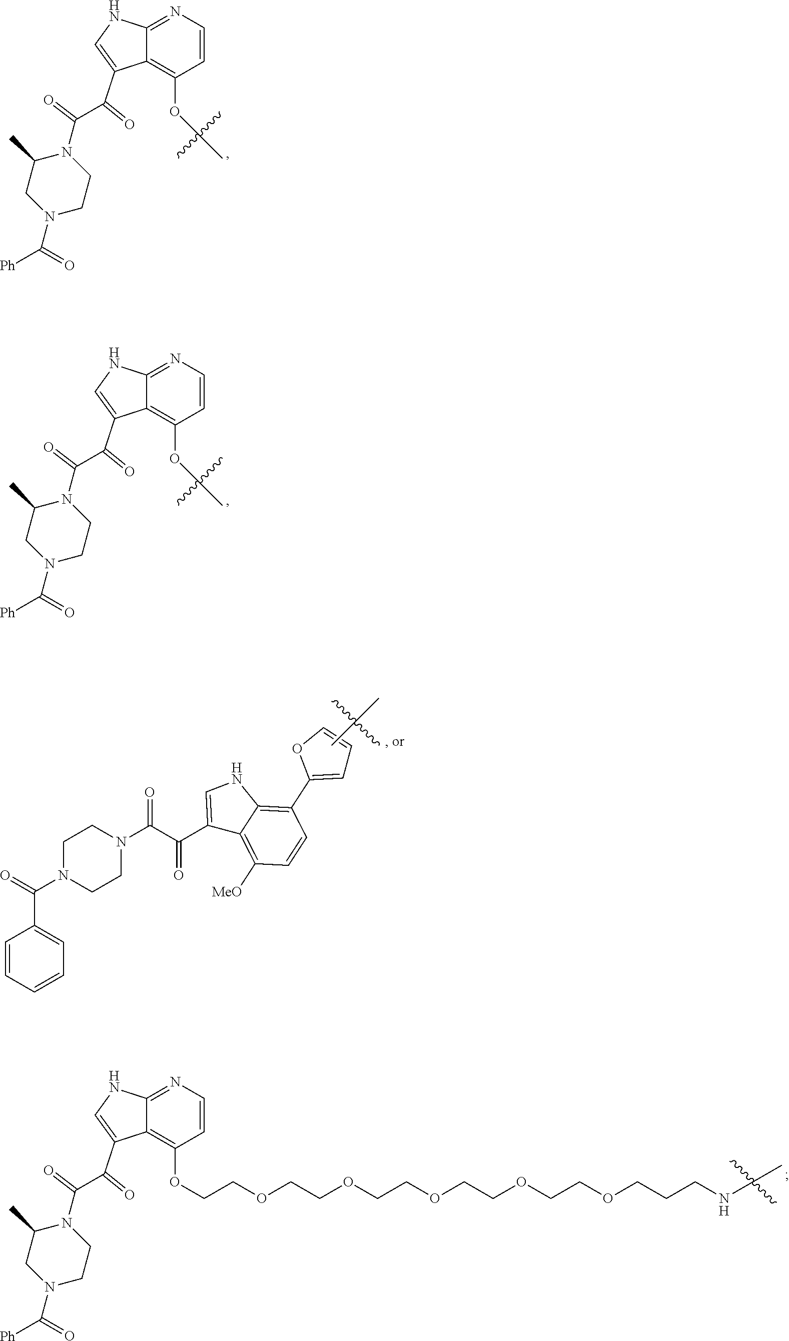
wherein the Protein binder that binds to MIF comprises: 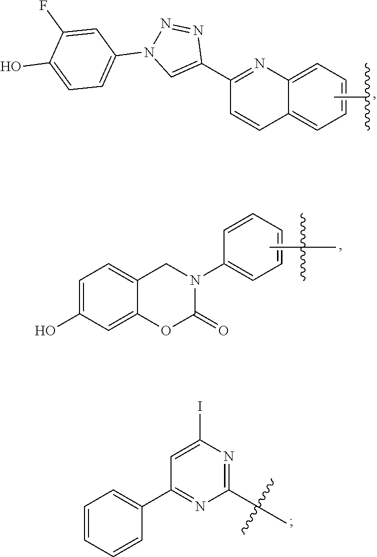
wherein the Protein binder that binds to IgA comprises:
(SEQ ID NO: 70)
STFCLLGQKDQSYCFTI
(SEQ ID NO: 71)
HMRCLHYKGRRVCFLL,
(SEQ ID NO: 72)
KTMCLRYNHDKVCFRI,
(SEQ ID NO: 73)
LVLCL VHRTSKHRKCFVI,
A2-3a:
(SEQ ID NO: 75)
SDVCLRYRGRPVCFQV,
Opt-1:
(SEQ ID NO: 76)
HMVCLAYRGRPVCFAL,
Opt-2:
(SEQ ID NO: 77)
HMVCLSYRGRPVCFSL,
Opt-3:
(SEQ ID NO: 78)
HQVCLSYRGRPVCFST,
(SEQ ID NO: 79)
RDVCLRYRGRPVCFQV,
(SEQ ID NO: 80)
HDVCLRYRGRPVCFQV,
(SEQ ID NO: 81)
ADVCLRYRGRPVCFQV,
(SEQ ID NO: 82)
SAVCLRYRGRPVCFQV,
(SEQ ID NO: 83)
SMVCLRYRGRPVCFQV,
(SEQ ID NO: 84)
SDRCLRYRGRPVCFQV,
(SEQ ID NO: 85)
SDACLRYRGRPVCFQV,
(SEQ ID NO: 86)
SDVCARYRGRPVCFQV,
(SEQ ID NO: 87)
SDVCLNYRGRPVCFQV,
(SEQ ID NO: 88)
SDVCLHYRGRPVCFQV,
(SEQ ID NO: 89)
SDVCLAYRGRPVCFQV,
(SEQ ID NO: 90)
SDVCLRARGRPVCFQV,
(SEQ ID NO: 91)
SDVCLRYAGRPVCFQV,
(SEQ ID NO: 92)
SDVCLRYRARPVCFQV,
(SEQ ID NO: 93)
SDVCLRYRGSPVCFQV,
(SEQ ID NO: 94)
SDVCLRYRGAPVCFQV,
(SEQ ID NO: 95)
SDVCLRYRGRRVCFQV,
(SEQ ID NO: 96)
SDVCLRYRGRAVCFQV,
(SEQ ID NO: 97)
SDVCLRYRGRPACFQV,
(SEQ ID NO: 98)
SDVCLRYRGRPVCRQV,
(SEQ ID NO: 99)
SDVCLRYRGRPVCAQV,
(SEQ ID NO: 100)
SDVCLRYRGRPVCFRV,
(SEQ ID NO: 101)
SDVCLRYRGRPVCFLV,
(SEQ ID NO: 102)
SDVCLRYRGRPVCFAV,
(SEQ ID NO: 103)
SDVCLRYRGRPVCFQW,
(SEQ ID NO: 104)
SDVCLRYRGRPVCFQL,
(SEQ ID NO: 105)
SDVCLRYRGRPVCFQA,
(SEQ ID NO: 106)
GRYQCQYRIGHYRFRYSD,
(SEQ ID NO: 107)
GRYQAQYRIGHYRFRYSD,
(SEQ ID NO: 108)
GRYQCQYRIGHYRFRYSD,
(SEQ ID NO: 109)
CLIPS-CHYRFRC,
(SEQ ID NO: 110)
CLIPS-CRIGHYRFRC,
(SEQ ID NO: 111)
CLIPS-YQACHYRFRC,
(SEQ ID NO: 119)
GRYQAQCRIGHYRFCYSD,
(SEQ ID NO: 112)
CLIPS-RYQAQCRIGHYRFC,
(SEQ ID NO: 113)
CLIPS-GRYQCQYRIGHYRFRYCD,
(SEQ ID NO: 114)
CLIPS-GRYQACYRIGHYRFRCSD,
(SEQ ID NO: 115)
CLIPS-GRYQAQCRIGHYRFCYSD,
(SEQ ID NO: 116)
RYQAQCRIGHYRFC,
(SEQ ID NO: 117)
GRYQCQYRIGHYRFRYCD,
(SEQ ID NO: 118)
GRYQACYRIGHYRFRCSD,

27. The compound of claim 1,
wherein the TNF binder comprises the amino acid sequence of at least one of:
(SEQ ID NO: 120)
STPTRYS,
(SEQ ID NO: 121)
CALWHWWHC,
(SEQ ID NO: 122)
C(T/S)WLHWWAC,
(SEQ ID NO: 123)
(L/M)HEL(Y/F)(L/M)X(W/Y/F),
(SEQ ID NO: 124)
D-DDDEK QLKER WYKRW LEYLD EFKKN,
(SEQ ID NO: 125)
D-TEEEK QLKEW WYKHW QEYLE EFKKN,
(SEQ ID NO: 126)
GACPPCLWQVLCGGSGSGSG,
(SEQ ID NO: 127)
HIHDDLLRYYGW linear,
or
(SEQ ID NO: 128)
tetra branched,
peptide,
(SEQ ID NO: 129)
KRWSRYF,
(SEQ ID NO: 127)
HIHDDLLRYYGW,
(SEQ ID NO: 130)
YCWSQYLCY,
(SEQ ID NO: 131)
DFLPHYKNTSLGHRP,
(SEQ ID NO: 132)
YCLYQSWCY;
or
wherein the TNF binder comprises at least one of:
TNFR1, TNFR2, P51, P52, anticachexin C1, anticachexin C2, adalimumab, infliximab, etanercept, golimumab, certolizumab;
or
wherein the TNF binder comprises at least one of: 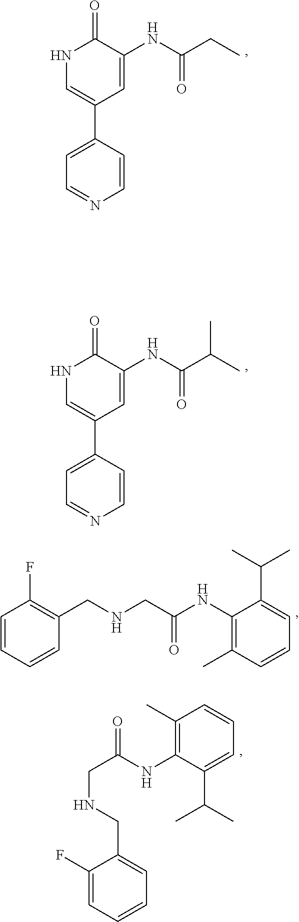 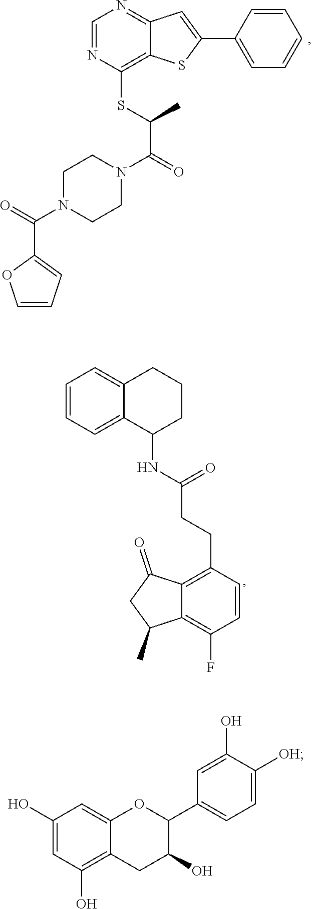 [SMILES: C[C@H](Sc1ncnc2cc(-c3ccccc3)sc12)C(=O)N1CCN(C(=O)c2ccco2)CC1 ; C[C@H]1CC(=O)c2c(CCC(=O)NC3CCCc4ccccc43)ccc(F)c21 ; Oc1cc(O)c2c(c1)OC(c1ccc(O)c(O)c1)[C@@H](O)C2]
or
wherein the TNF binder comprises at least one of: 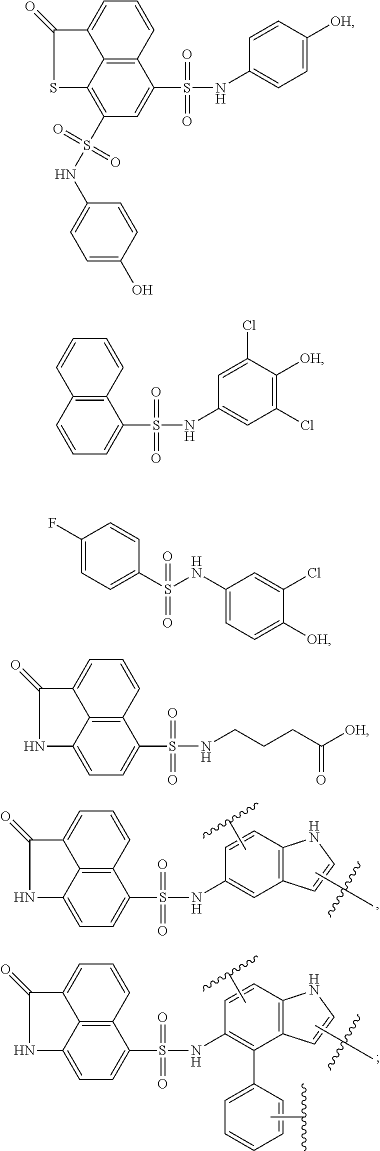
or
wherein the TNF binder comprises at least one of: 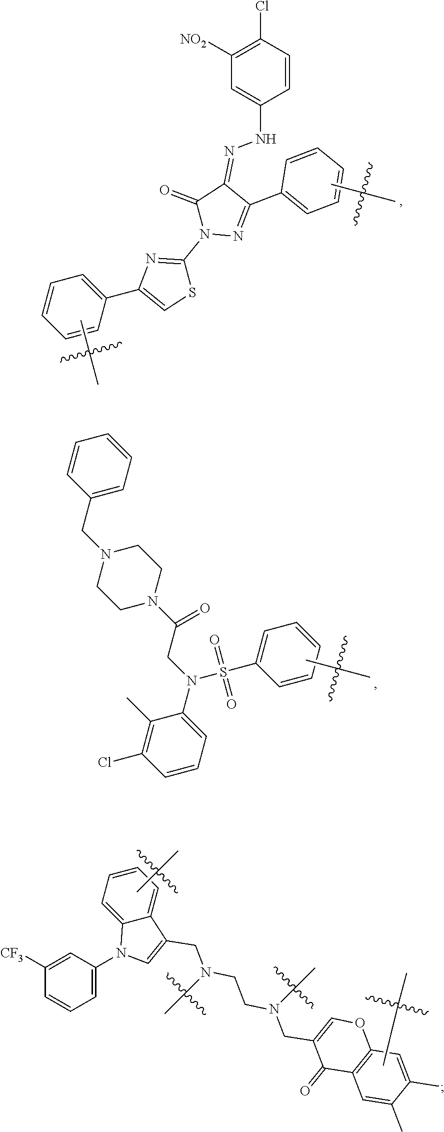
or
wherein the TNF binder comprises at least one of: 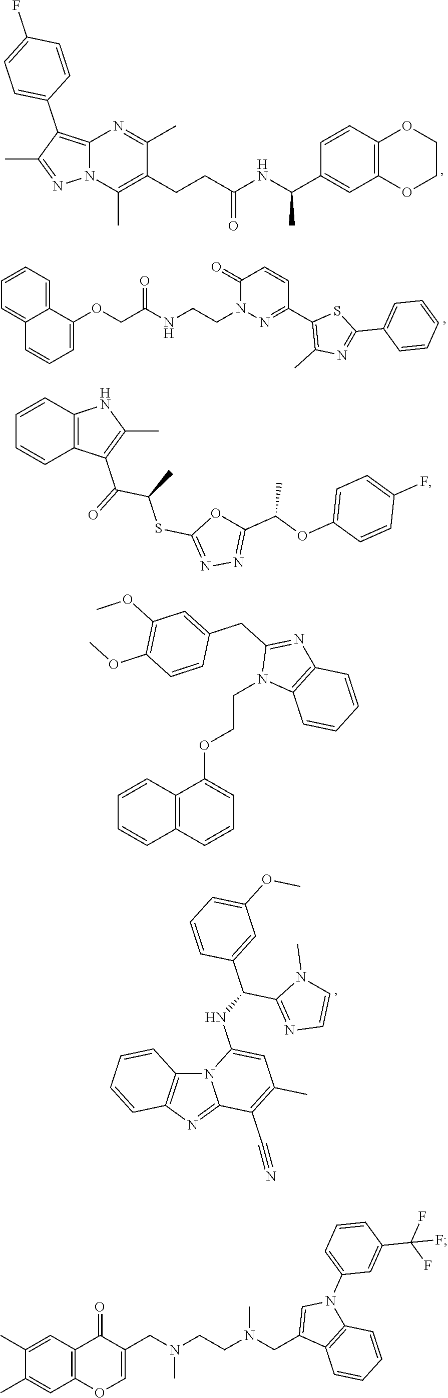
or
wherein the TNF binder comprises at least one of: 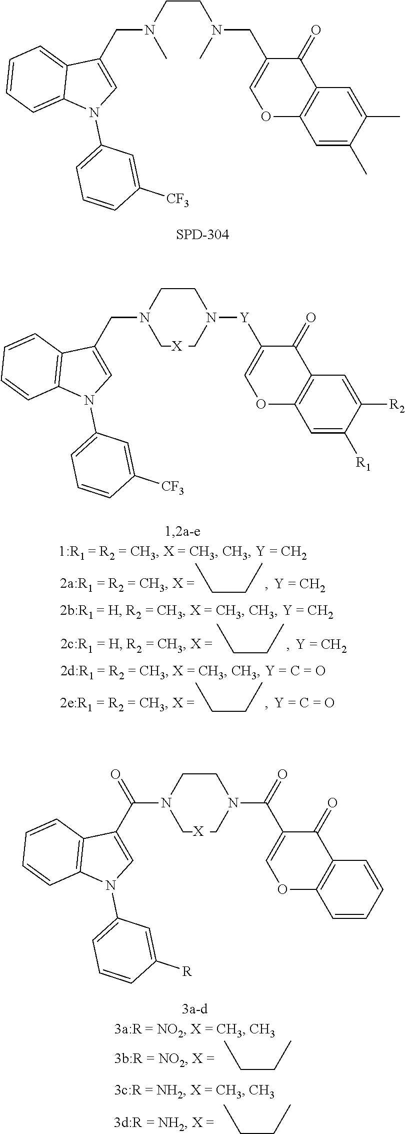 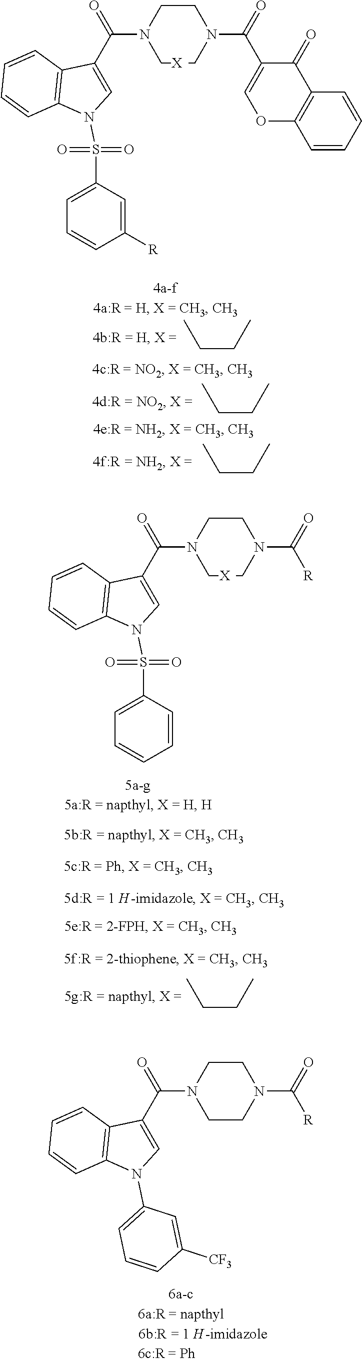 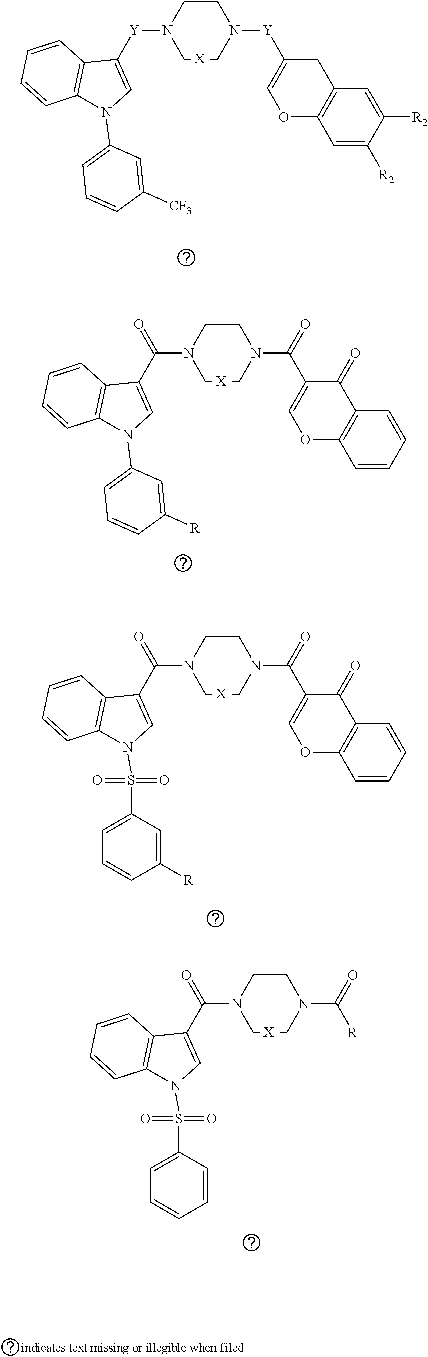 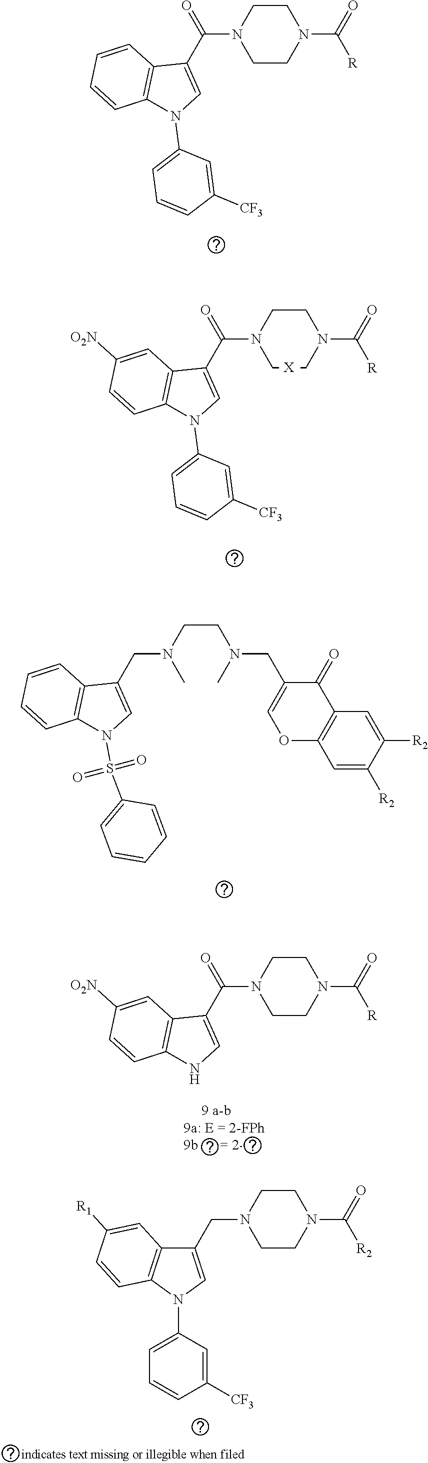 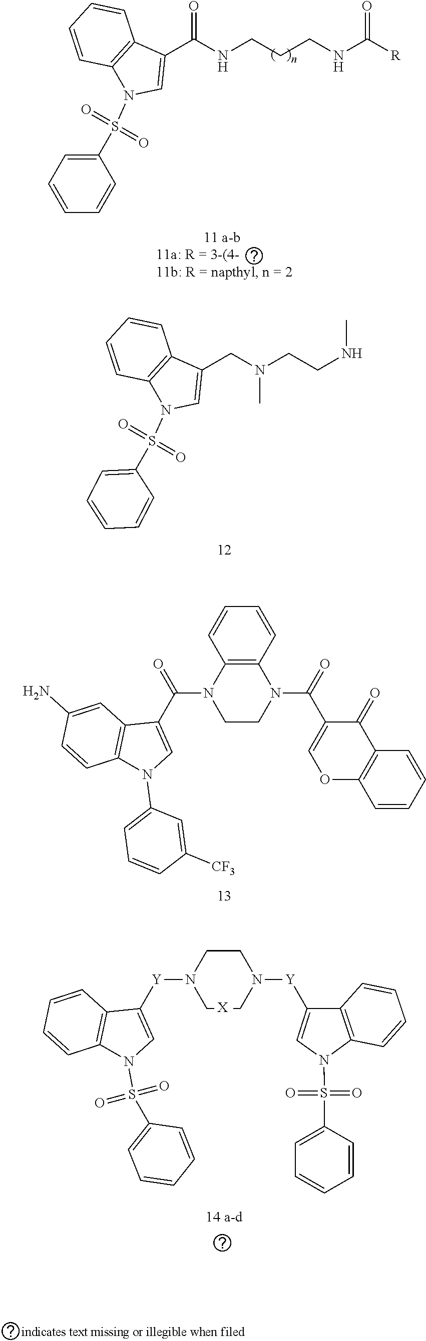 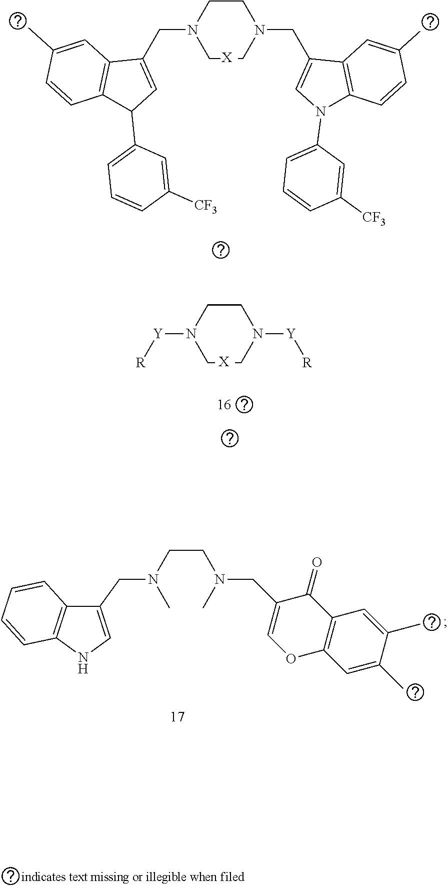
or
wherein the TNF binder comprises at least one of: 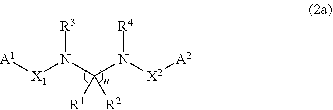
wherein:
A¹ and A² are independently a substituted or unsubstituted phenyl group, wherein the substituents comprise at least one of F, Cl, Br, I, OH, C₁-C₄ alkyl, C₁-C₄ alkyl substituted with at least one OH, C₁-C₄ fluoroalkyl, C₁-C₄ alkoxy, C₁-C₄ haloalkoxy, benzyloxy, and the following heterocyclic rings optionally substituted with at least one of F, Cl, Br, I, OH, C₁-C₄ alkyl, C₁-C₄ alkyl substituted with at least one OH, C₁-C₄ fluoroalkyl, C₁-C₄ alkoxy, and C₁-C₄ haloalkoxy, or 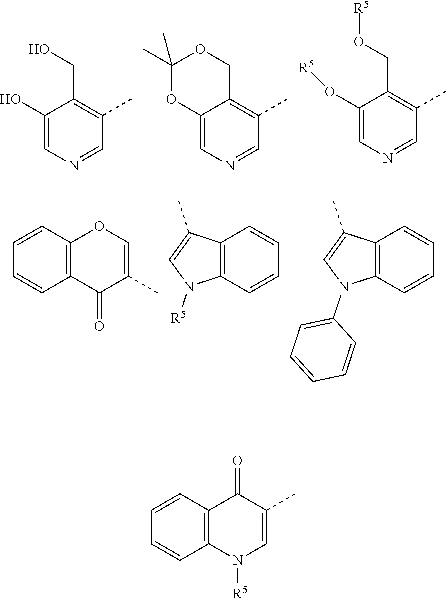
(dotted lines indicate point of attachment);
each R⁵ is independently hydrogen or optionally substituted C₁-C₄ alkyl;
R¹ and R² are independently hydrogen or optionally substituted C₁-C₄ alkyl;
X¹ and X² are independently carbonyl or CH₂;
n is 2, 3, or 4;
R³ and R⁴ are independently hydrogen or optionally substituted C₁-C₄ alkyl, or R³ and R⁴ can combine to form a heterocyclyl ring;
or
wherein the TNF binder comprises at least one of IA-14069, 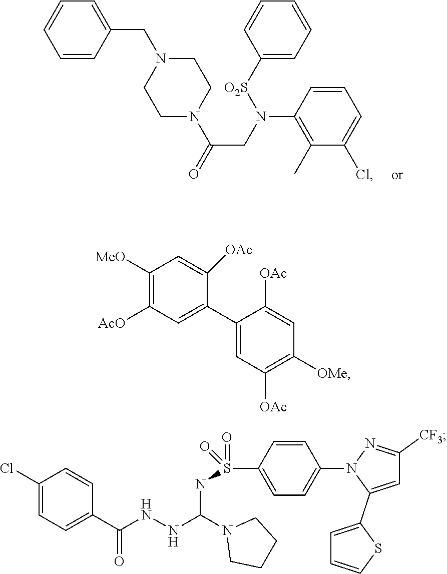
wherein the TNF binder comprises at least one of: 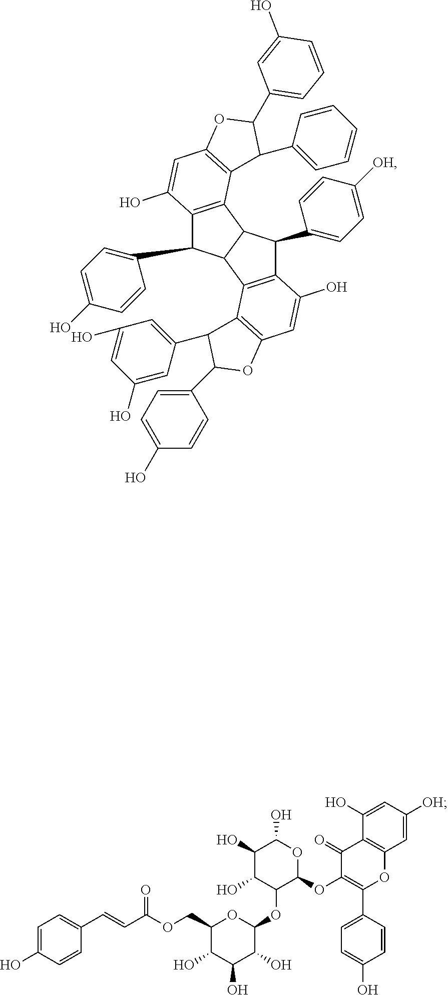 [SMILES: Oc1ccc(C2Oc3cc(O)c4c(c3C2c2cc(O)cc(O)c2)C2C(c3c5c(cc(O)c3[C@@H]2c2ccc(O)cc2)OC(c2cccc(O)c2)C5c2ccccc2)[C@H]4c2ccc(O)cc2)cc1 ; O=C(/C=C/c1ccc(O)cc1)OC[C@H]1O[C@@H](OC2[C@H](Oc3c(-c4ccc(O)cc4)oc4cc(O)cc(O)c4c3=O)O[C@@H](O)[C@H](O)[C@H]2O)[C@H](O)[C@@H](O)[C@@H]1O]
or
wherein the TNF binder comprises at least one of: 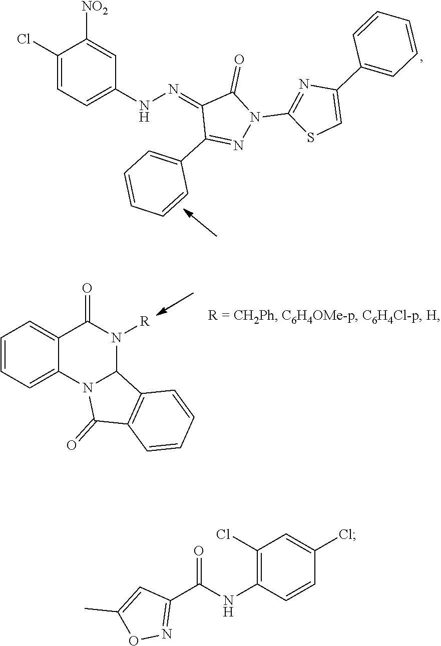
or
wherein the TNF binder comprises at least one of:

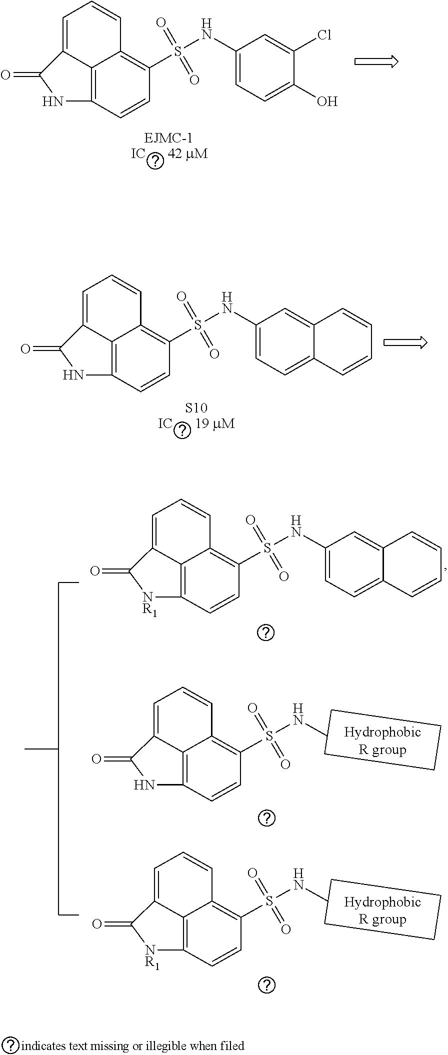

| Com- | |
|---|---|
| pound | Structure |
| EJMC-1 | 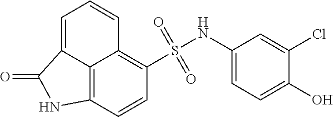 [SMILES: O=C1Nc2ccc(S(=O)(=O)Nc3ccc(O)c(Cl)c3)c3cccc1c23] |
| S10 | 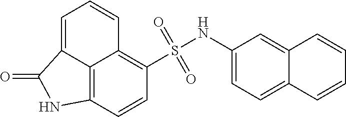 [SMILES: O=C1Nc2ccc(S(=O)(=O)Nc3ccc4ccccc4c3)c3cccc1c23] |
| S21 | 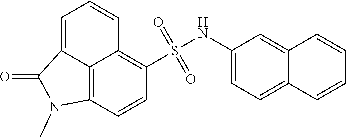 [SMILES: CN1C(=O)c2cccc3c(S(=O)(=O)Nc4ccc5ccccc5c4)ccc1c23] |
| S22 | 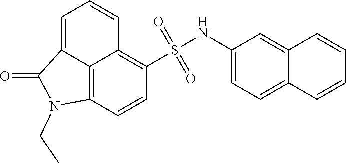 [SMILES: CCN1C(=O)c2cccc3c(S(=O)(=O)Nc4ccc5ccccc5c4)ccc1c23] |
| S23 | 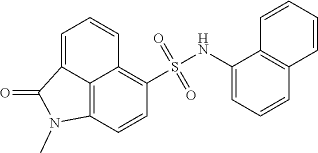 [SMILES: CN1C(=O)c2cccc3c(S(=O)(=O)Nc4cccc5ccccc45)ccc1c23] |
| S24 | 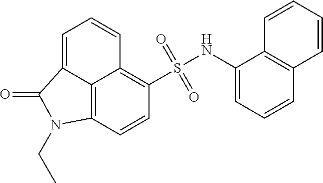 [SMILES: CCN1C(=O)c2cccc3c(S(=O)(=O)Nc4cccc5ccccc45)ccc1c23] |
| S25 | 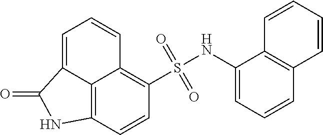 [SMILES: O=C1Nc2ccc(S(=O)(=O)Nc3cccc4ccccc34)c3cccc1c23] |
| S26 | 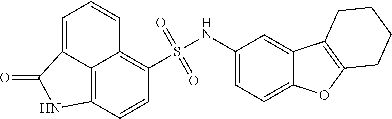 [SMILES: O=C1Nc2ccc(S(=O)(=O)Nc3ccc4oc5c(c4c3)CCCC5)c3cccc1c23] |
| S27 | 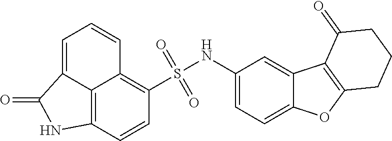 [SMILES: O=C1CCCc2oc3ccc(NS(=O)(=O)c4ccc5c6c(cccc46)C(=O)N5)cc3c21] |
| 4a | 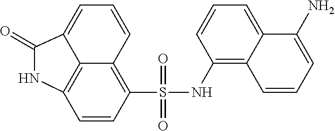 [SMILES: Nc1cccc2c(NS(=O)(=O)c3ccc4c5c(cccc35)C(=O)N4)cccc12] |
| 4c | 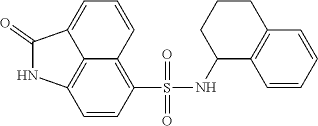 [SMILES: O=C1Nc2ccc(S(=O)(=O)NC3CCCc4ccccc43)c3cccc1c23] |
| 4d | 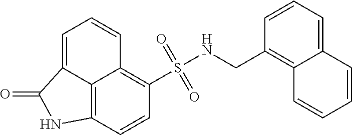 [SMILES: O=C1Nc2ccc(S(=O)(=O)NCc3cccc4ccccc34)c3cccc1c23] |
| 4e | 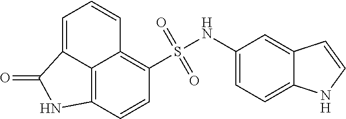 [SMILES: O=C1Nc2ccc(S(=O)(=O)Nc3ccc4[nH]ccc4c3)c3cccc1c23] |
| 4r | 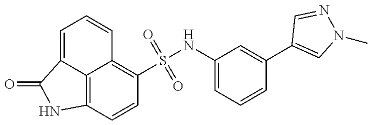 [SMILES: Cn1cc(-c2cccc(NS(=O)(=O)c3ccc4c5c(cccc35)C(=O)N4)c2)cn1] |
| 4g | 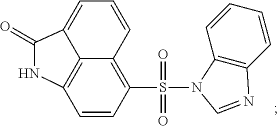 [SMILES: O=C1Nc2ccc(S(=O)(=O)n3cnc4ccccc43)c3cccc1c23] |

or
wherein the TNF binder comprises at least one of: 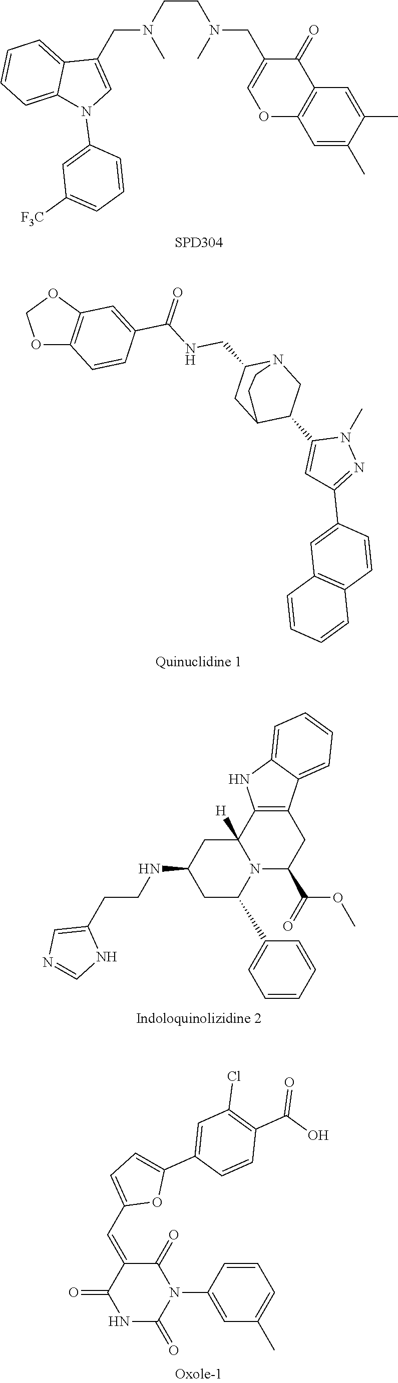 [SMILES: Cc1cc2occ(CN(C)CCN(C)Cc3cn(-c4cccc(C(F)(F)F)c4)c4ccccc34)c(=O)c2cc1C ; "SPD304" ; Cn1nc(-c2ccc3ccccc3c2)cc1[C@H]1CN2CCC1C[C@@H]2CNC(=O)c1ccc2c(c1)OCO2 ; COC(=O)[C@@H]1Cc2c([nH]c3ccccc23)[C@H]2C[C@H](NCCc3cnc[nH]3)C[C@@H](c3ccccc3)N21 ; Cc1cccc(N2C(=O)NC(=O)/C(=C/c3ccc(-c4ccc(C(=O)O)c(Cl)c4)o3)C2=O)c1] 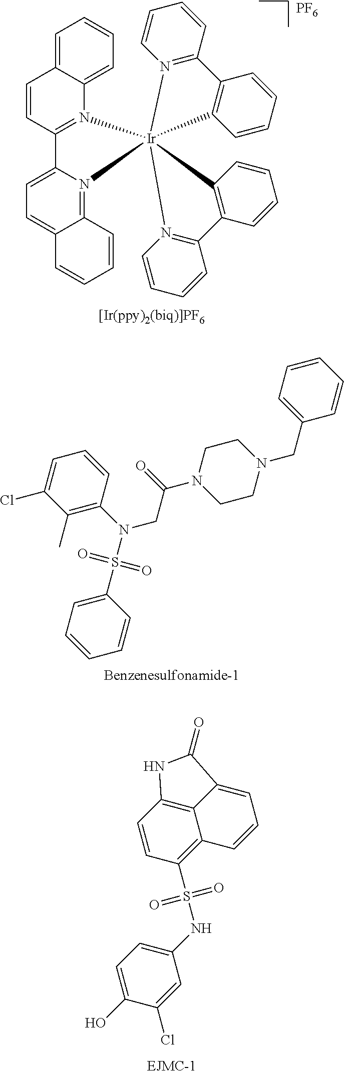 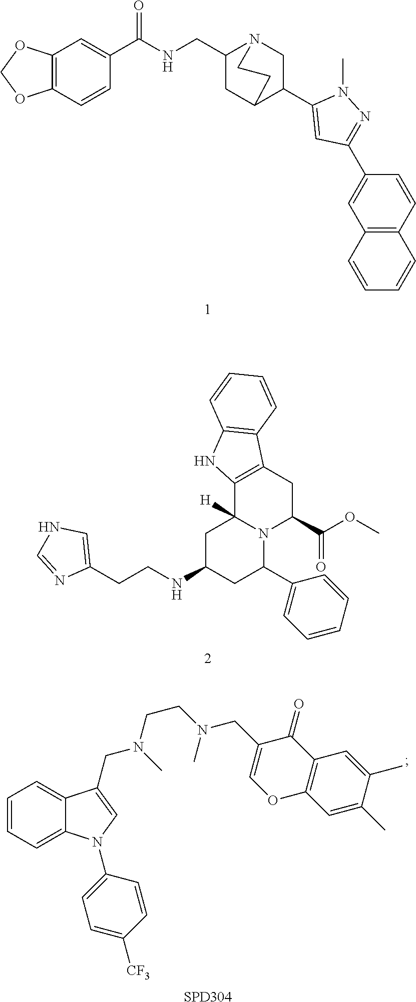 [SMILES: Cn1nc(-c2ccc3ccccc3c2)cc1C1CN2CCC1CC2CNC(=O)c1ccc2c(c1)OCO2 ; COC(=O)[C@@H]1Cc2c([nH]c3ccccc23)[C@H]2C[C@H](NCCc3c[nH]cn3)CC(c3ccccc3)N21 ; Cc1cc2occ(CN(C)CCN(C)Cc3cn(-c4ccc(C(F)(F)F)cc4)c4ccccc34)c(=O)c2cc1C ; "SPD304"]
or
wherein the TNF binder comprises at least one of: 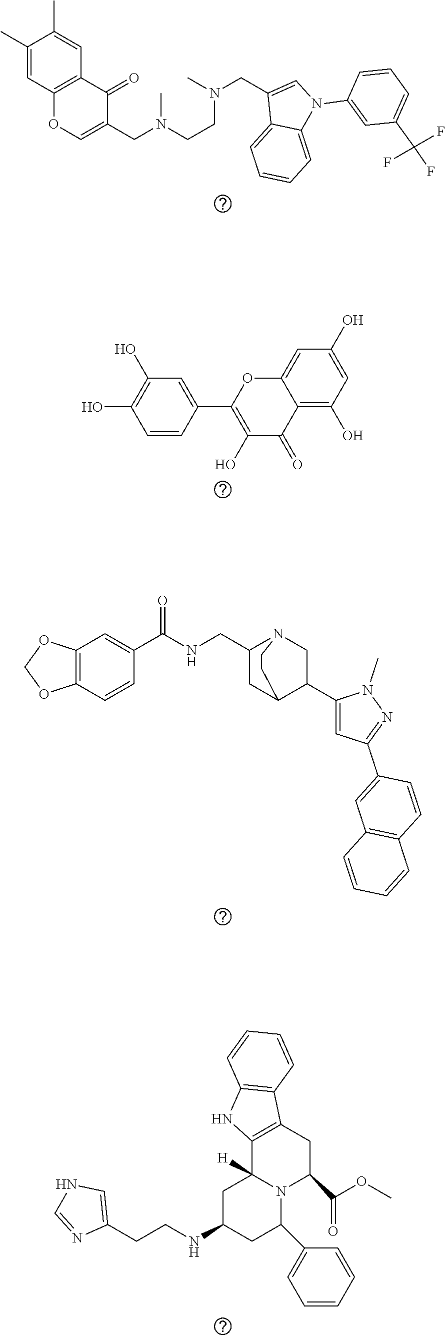 [SMILES: Cc1cc2occ(CN(C)CCN(C)Cc3cn(-c4cccc(C(F)(F)F)c4)c4ccccc34)c(=O)c2cc1C ; O=c1c(O)c(-c2ccc(O)c(O)c2)oc2cc(O)cc(O)c12 ; Cn1nc(-c2ccc3ccccc3c2)cc1C1CN2CCC1CC2CNC(=O)c1ccc2c(c1)OCO2 ; COC(=O)[C@@H]1Cc2c([nH]c3ccccc23)[C@H]2C[C@H](NCCc3c[nH]cn3)CC(c3ccccc3)N21] 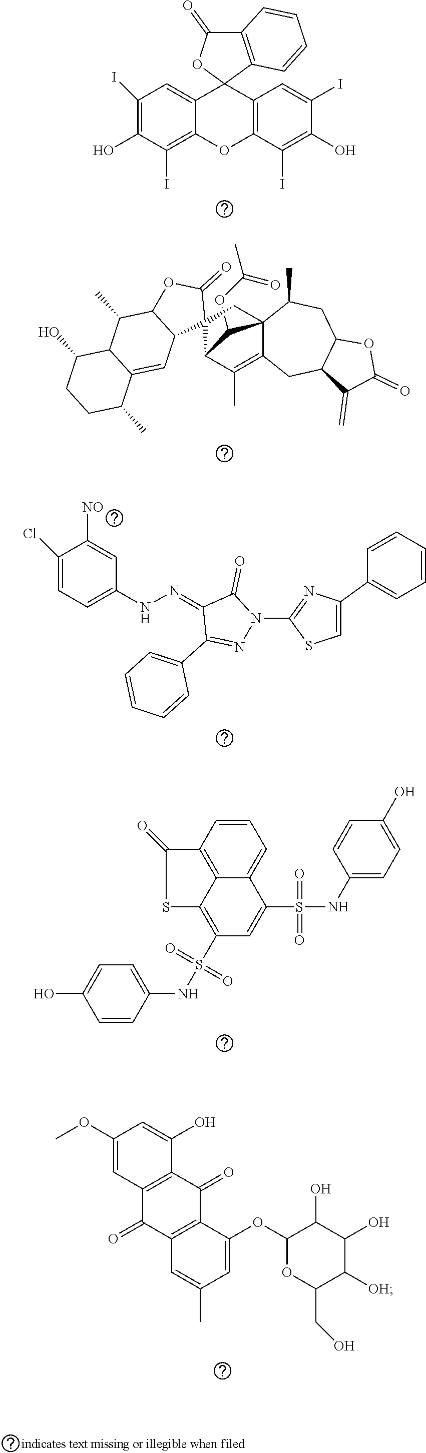
or
wherein the TNF binder comprises at least one of: 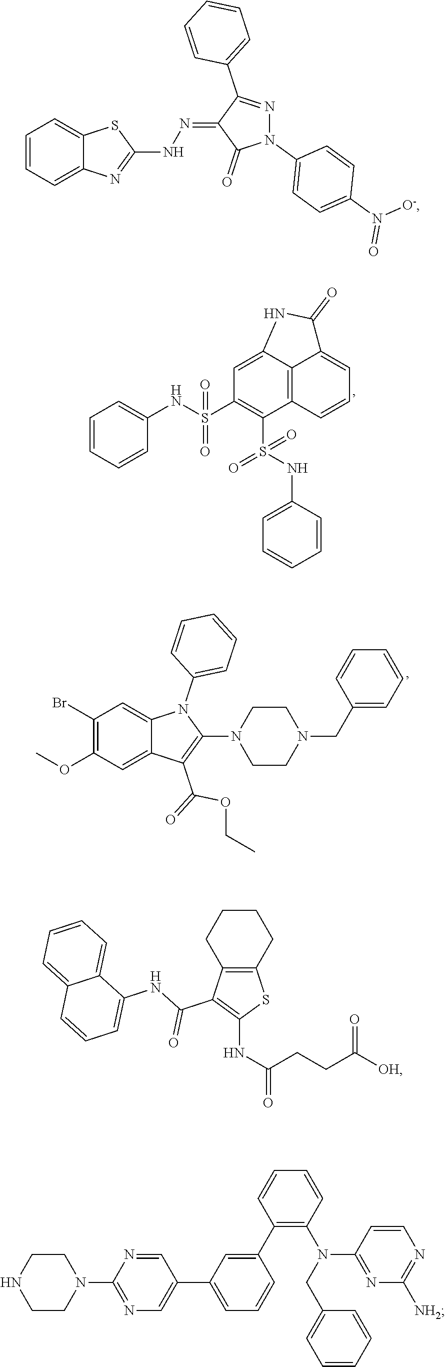
wherein the TNF binder comprises at least one of: 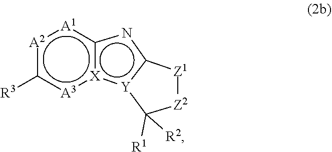
wherein:
R¹ is H, OH, F or optionally substituted (C₁-C₃)alkyl;
R² is optionally substituted aryl, optionally substituted (C₃-C₈)cycloalkyl, optionally substituted heteroaryl or optionally substituted heterocyclyl; or
R¹ and R² together can form an optionally substituted saturated or partially saturated carbocyclic ring or optionally substituted saturated or partially saturated heterocyclic ring;
up to two of A¹, A², and A³ are N, and the rest are independently C(R^{A2});
X is N and Y is C, wherein:
Z¹ is C(R^{z})₂ and Z² is C(R^{z})₂—, —N(R^{z1})— or —O—; or
Z¹ is CH₂ and Z² is Z^{2a}—Z^{2b}-,
wherein Z^{2a} is attached to Z¹ and Z^{2b} is attached to C(R¹)(R²); and Z^{2a} and Z^{2b} are independently C(R^{z})₂—, C(R^{z})₂C(R^{z})₂—, —O- or N(R^{z1}) provided that one of Z^{2a} and Z^{2b} is C(R^{z})₂ or C(R^{z})₂C(R^{z})₂; or Z^{2a}—Z^{2b}— form —N(R^{z1})C(O)— or C(O)N(R^{z1})—;
or
X is C and Y is N, provided that R¹ is not OH or F, wherein:
Z¹ is C(R^{z})₂ and Z² is C(R^{z})₂—; or
Z¹ is C(R^{z})₂ and Z² is Z^{2a}—Z^{2b}-,
wherein Z^{2a} is attached to Z¹ and Z^{2b} is attached to C(R¹)(R²), and Z^{2a} is C(R^{z})₂—, —C(R^{z})₂C(R^{z})₂—, —O- or N(R^{z1}), and Z^{2b} is C(R^{z})₂—, or Z^{2a}—Z^{2b} form —N(R^{z1})C(O)— or —C(O)N(R^{z1})—;
R³ is —R^{3a}—R^{3b}, wherein:
R^{3a} is an optionally substituted aryl, optionally substituted saturated or partially saturated heterocyclyl or optionally substituted heteroaryl;
R^{3b} is H, —CF₃, CN, C(O)OH, N(R^{a})(R^{b}), C(O)N(R^{a})(R^{b}), C(O)-optionally substituted heterocyclyl, O(R^{a}), S(O)₂(C₁-C₃)alkyl, S(O)₂N(R^{c})(R^{d}), S—(C₁-C₃)alkyl, S(O)₂—R^{c} optionally substituted (C₁-C₅)alkyl, (CH₂)ₚ-optionally substituted (C₃-C₆)cycloalkyl, (CH₂)ₚ-optionally substituted heteroaryl or (CH₂)ₚ-optionally substituted saturated, unsaturated or partially saturated heterocyclyl; provided that R^{3b} is not H or methoxy when R² is optionally substituted phenyl;
R^{a} and R^{b} are independently selected from H, optionally substituted (C₁-C₅)alkyl, C(O) optionally substituted (C₁-C₅)alkyl, optionally substituted (CH₂)ₚ-(C₃-C₆)cycloalkyl and (CH₂)ₚ-optionally substituted heterocyclyl;
R^{c} and R^{d} are independently selected from H, optionally substituted (C₁-C₅)alkyl, optionally substituted (CH₂)ₚ-(C₃-C₆)cycloalkyl and (CH₂)ₚ-optionally substituted heterocyclyl;
R^{A2} is independently H, CF₃, halo or (C₁-C₃)alkyl;
R^{z} is independently H, F, CF₃, OH or (C₁-C₃)alkyl;
R^{z1} is independently H or (C₁-C₃)alkyl; and
p is independently 0, 1 or 2;
or
wherein the TNF binder comprises at least one of: 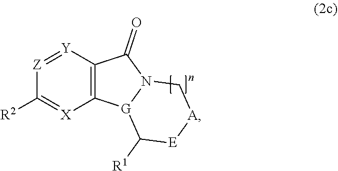
wherein:
X, Y, and Z are independently CR⁴ or N; provided that Y and Z are not both N;
A is —C(R^{z})₂—;
E is CH₂ or O and G is CH; or E is CH₂ and G is CH or N;
R¹ is optionally substituted aryl or optionally substituted heteroaryl;
R² is —R^{2a}—R^{2b}, wherein:
R^{2a} is an optionally substituted saturated, unsaturated or partially saturated heterocyclyl or optionally substituted heteroaryl;
R^{2b} is —N(R^{a})(R^{b}), —O(R^{a}), optionally substituted (C₁-C₅)alkyl, optionally substituted (C₃-C₆)cycloalkyl, (CH₂)ₚ-optionally substituted heteroaryl or (CH₂)ₚ-optionally substituted heterocyclyl; wherein
R^{a} and R^{b} are independently selected from H, optionally substituted (C₁-C₅)alkyl, and (CH₂)ₙ-optionally substituted heterocyclyl;
R⁴ is independently H, halo, CF₃, or (C₁-C₃)alkyl;
R^{z} is independently H, halo, CF₃, or (C₁-C₃)alkyl;
n is 0 or 1; and
p is 0 or 1.

28. The compound of claim 27,
wherein the compound of formula (Ia) comprises one of the following: 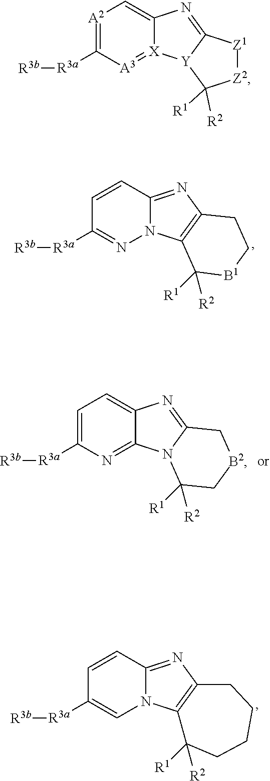
wherein:
A² is CH or N; A³ is CH or N; B¹ is CH₂ or O; B² is CH₂ or O; X is C or N; Y is C or N;
Z is CH₂ or O; and Z² is CH₂ or O;
R^{3a} is selected from the group consisting of: 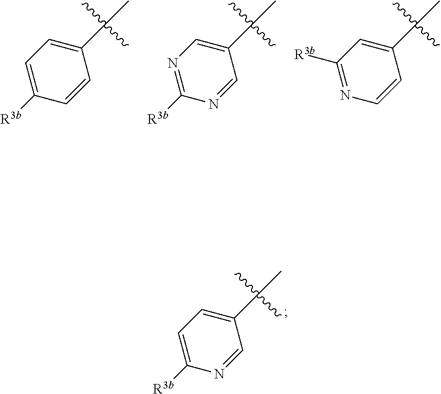
R^{3b} is selected from the group consisting of: 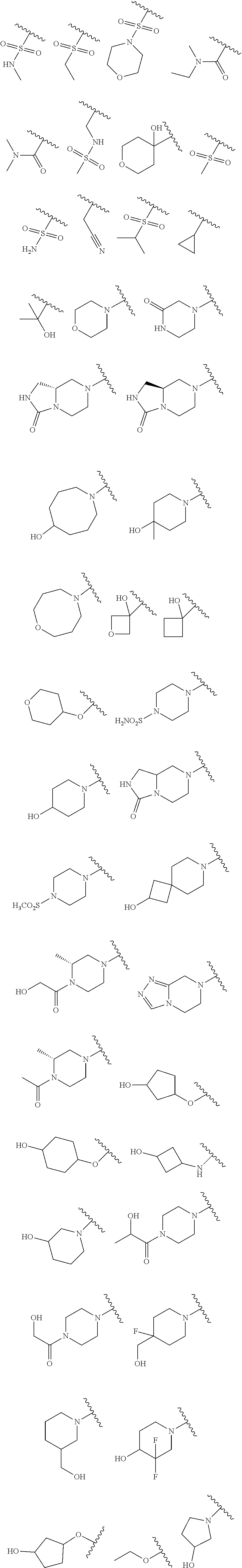 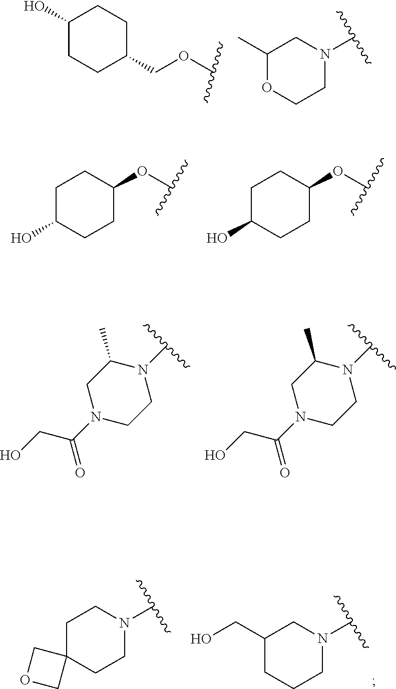
wherein the compound of formula (2a) comprises 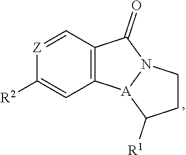
wherein G is N or CH; Z is CH or CF; R¹ is selected from the group consisting of 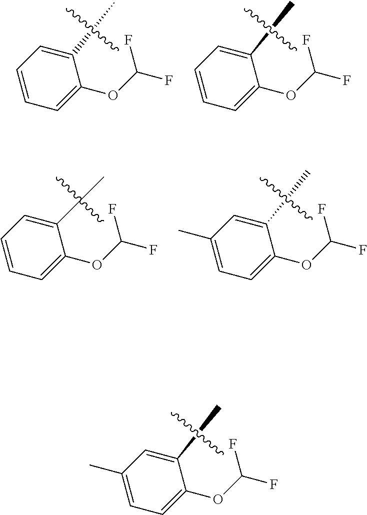
R² is selected from the group consisting of

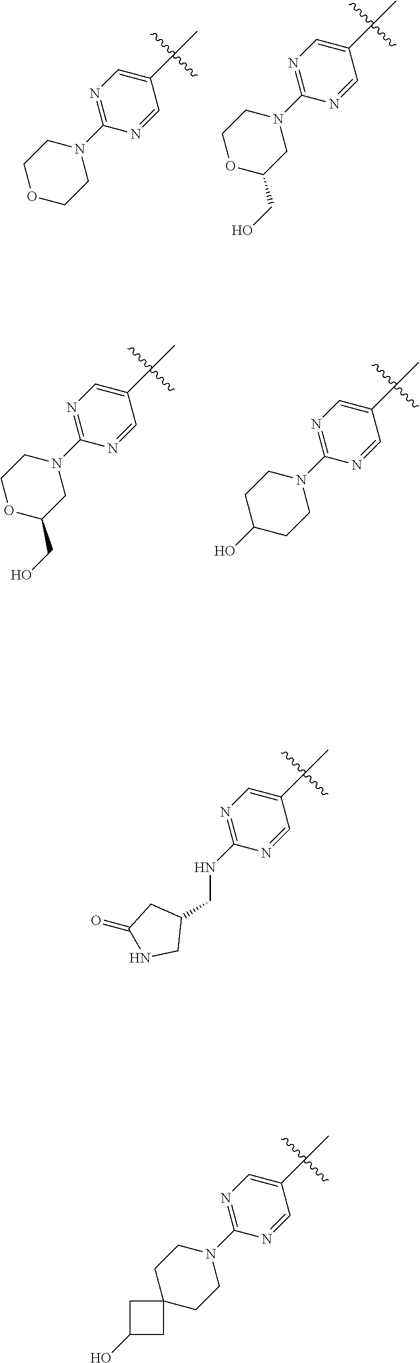

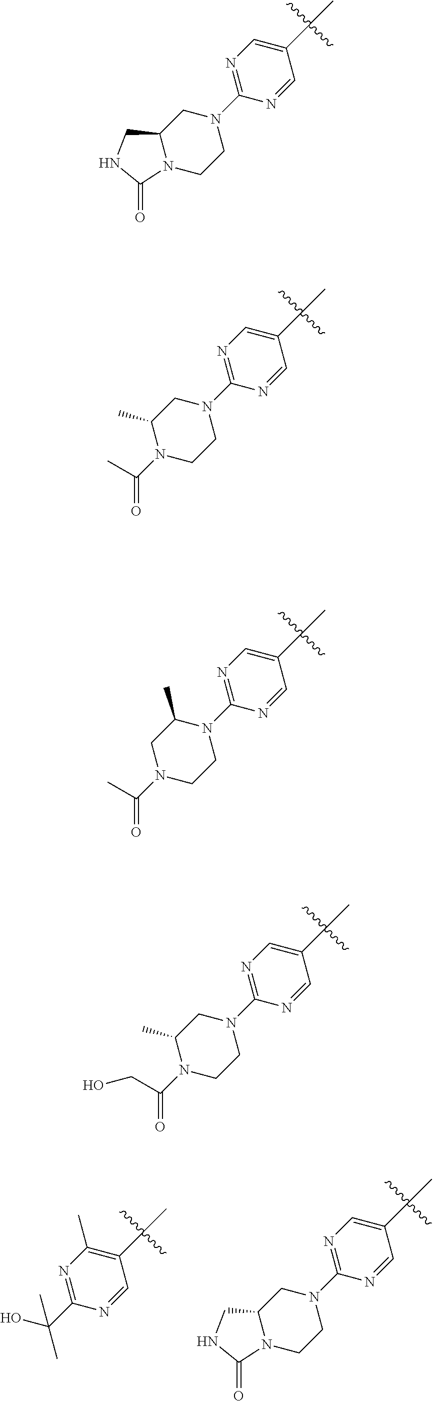 [SMILES: *c1cnc(N2CCN3C(=O)NC[C@H]3C2)nc1 ; *c1cnc(N2CCN(C(C)=O)[C@H](C)C2)nc1 ; *c1cnc(N2CCN(C(C)=O)C[C@H]2C)nc1 ; *c1cnc(N2CCN(C(=O)CO)[C@H](C)C2)nc1 ; *c1cnc(C(C)(C)O)nc1C ; *c1cnc(N2CCN3C(=O)NC[C@@H]3C2)nc1]

29. The compound of claim 1, wherein the AATM comprises one of the following:
a FcRn antagonist, 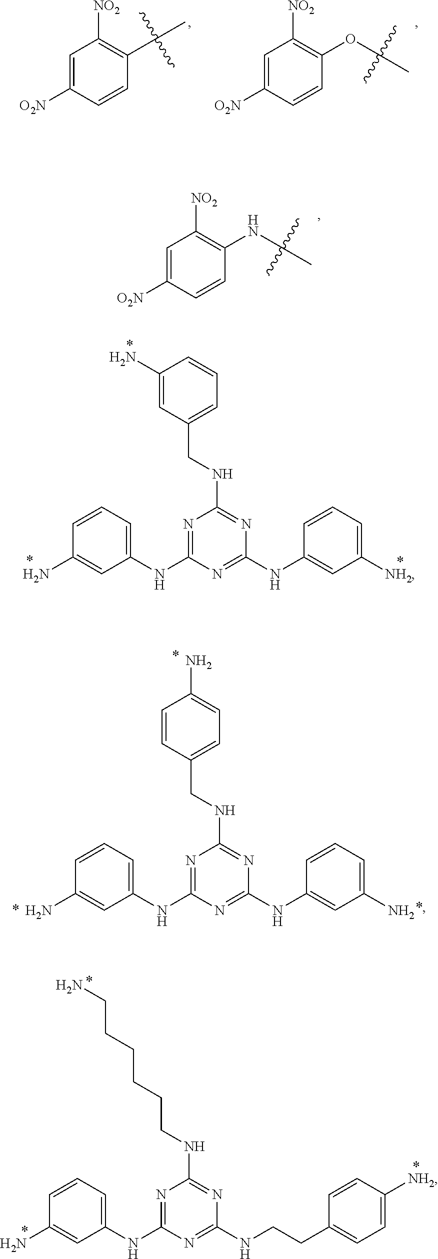 [SMILES: *c1ccc([N+](=O)[O-])cc1[N+](=O)[O-] ; *Oc1ccc([N+](=O)[O-])cc1[N+](=O)[O-] ; *Nc1ccc([N+](=O)[O-])cc1[N+](=O)[O-] ; Nc1cccc(CNc2nc(Nc3cccc(N)c3)nc(Nc3cccc(N)c3)n2)c1 ; Nc1ccc(CNc2nc(Nc3cccc(N)c3)nc(Nc3cccc(N)c3)n2)cc1 ; NCCCCCCNc1nc(NCCc2ccc(N)cc2)nc(Nc2cccc(N)c2)n1] 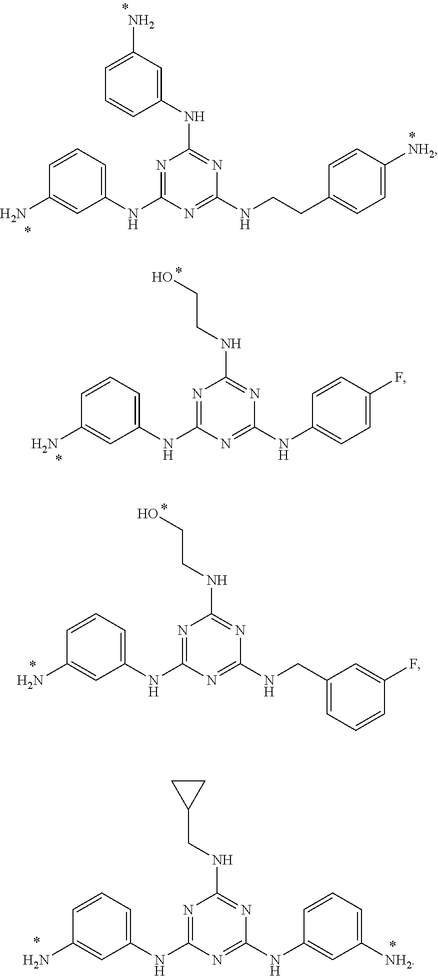 [SMILES: Nc1ccc(CCNc2nc(Nc3cccc(N)c3)nc(Nc3cccc(N)c3)n2)cc1 ; Nc1cccc(Nc2nc(NCCO)nc(Nc3ccc(F)cc3)n2)c1 ; Nc1cccc(Nc2nc(NCCO)nc(NCc3cccc(F)c3)n2)c1 ; Nc1cccc(Nc2nc(NCC3CC3)nc(Nc3cccc(N)c3)n2)c1]
cyclic peptide FcIII, or any reduced form thereof,
cyclic peptide FcIII-4C (amide), or any reduced form thereof,
a compound of formula (3a) or (3b): 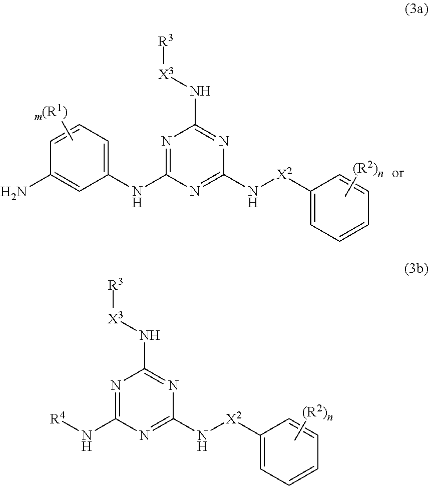
wherein:
each occurrence of R¹ is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, or —N(R)C(═O)N(R)₂, wherein each occurrence of R is independently H, C₁-C₆ alkyl, or C₃-C₈ cycloalkyl;
m is 0, 1, 2, 3, or 4;
X² is a bond, optionally substituted C₁-C₆ alkyl, or optionally substituted C₁-C₆ heteroalkyl;
each occurrence of R² is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, or —N(R)C(═O)N(R)₂, wherein each occurrence of R is independently H, C₁-C₆ alkyl, or C₃-C₈ cycloalkyl;
n is 0, 1, 2, 3, or 4;
X³ is a bond, optionally substituted C₁-C₆ alkyl, or optionally substituted C₁-C₆ heteroalkyl;
R³ is H, R, —OH, —NH₂, —NHR, —C(═O)OH, or —SH, wherein each occurrence of R is C₁-C₆ alkyl or C₃-C₈ cycloalkyl;
R⁴ is cycloalkyl, which is optionally substituted with 1-4 groups independently selected from the group consisting of F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, and —N(R)C(═O)N(R)₂, wherein each occurrence of R is independently H, C₁-C₆ alkyl, or C₃-C₈ cycloalkyl;
a compound of formula: 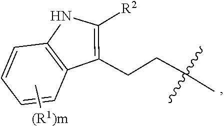
wherein:
each occurrence of R¹ is independently F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, or —N(R)C(═O)N(R)₂, wherein each occurrence of R is independently H, C₁-C₆ alkyl, or C₃-C₈ cycloalkyl;
m is 0, 1, 2, 3, or 4;
each occurrence of R² is independently H, F, Cl, Br, I, CN, NO₂, R, OR, C₁-C₆ haloalkyl, C₃-C₈ halocycloalkyl, C₁-C₆ haloalkoxy, C₃-C₈ halocycloalkoxy, —N(R)₂, —SR, —S(═O)R, —S(═O)₂R, —S(═O)₂N(R)₂, —C(═O)R, —C(═O)OR, —OC(═O)R, —C(═O)N(R)₂, —N(R)S(═O)₂R, —N(R)C(═O)OR, —N(R)C(═O)R, or —N(R)C(═O)N(R)₂, wherein each occurrence of R is independently H, C₁-C₆ alkyl, or C₃-C₈ cycloalkyl.

30. The compound of claim 29, wherein the FcRn antagonist comprises rozanolixizumab or efgartigimod.

31. A pharmaceutical composition comprising at least one pharmaceutically acceptable excipient and at least one compound of claim 1, optionally further comprising another therapeutically active agent that treats, ameliorates, or prevents a disease or disorder.

32. (canceled)

33. A method of treating, ameliorating, or preventing a disease or disorder in a subject, the method comprising administering a therapeutically effective amount of at least one compound of claim 1, optionally wherein the subject is further administered at least one additional therapeutic agent that treats, ameliorates, or prevents the disease or disorder.

34. The method of claim 33, wherein the disease or disorder comprises an autoimmune disease, cancer, or inflammation.

35. The method of claim 34,
wherein the autoimmune disease comprises at least one of Addison's Disease, Autoimmune polyendodrine syndrome (APS) types 1, 2 and 3, autoimmune pancreatitis (ATP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (APlS), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymyositis, acute disseminated encephalomyelitis (ADEM), acute motor axonic neuropathy, anti-NMDA receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Meniere's disease, Behçet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, and pyoderma gangrenosum,
the cancer comprises at least one of prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx cancer, oesophagus cancer, larynx cancer, kidney cancer, or lymphoma:
the inflammation comprises at least one of inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, and arteriosclerosis.

36-38. (canceled)

39. The method of claim 33, wherein the subject is a mammal, optionally wherein the subject is a human.

40. (canceled)


SEQUENCE LISTING (140 sequences)

SEQ ID NO: 1 - protein, 17 aa, Artificial Sequence
  Chemically synthesized
  misc_feature (1)..(1): N-acetylated
  misc_feature (11)..(11): Xaa = norleucin
  misc_feature (17)..(17): terminus is C(=O)NH2
VKFNKPFVFLXIEQNTK

SEQ ID NO: 2 - protein, 17 aa, Artificial Sequence
  Chemically synthesized
VKFNKPFVFLMIEQNTK

SEQ ID NO: 3 - protein, 20 aa, Artificial Sequence
  Chemically synthesized
TWPKHFDKHTFYSILKLGKH

SEQ ID NO: 4 - protein, 19 aa, Artificial Sequence
  Chemically synthesized
TFFYGGSRGKRNNFKTEEY

SEQ ID NO: 5 - protein, 30 aa, Artificial Sequence
  Chemically synthesized
LRKLRKRLLRDADDLLRKLRKRLLRDADDL

SEQ ID NO: 6 - protein, 20 aa, Artificial Sequence
  Chemically synthesized
TEELRVRLASHLRKLRKRLL

SEQ ID NO: 7 - protein, 12 aa, Artificial Sequence
  Chemically synthesized
EAKIEKHNHYQK

SEQ ID NO: 8 - protein, 22 aa, Artificial Sequence
  Chemically synthesized
EAKIEKHNHYQKQLEIAHEKLR

SEQ ID NO: 9 - protein, 19 aa, Artificial Sequence
  Chemically synthesized
TFFYGGSRGKRNNFKTEEY

SEQ ID NO: 10 - protein, 8 aa, Artificial Sequence
  Chemically synthesized
  misc_feature (1)..(1): D-amino acid
  misc_feature (1)..(8): Cys and Pen form a ring
  misc_feature (3)..(3): Xaa = thiazolidine-4-carboxylic acid
  misc_feature (8)..(8): Xaa = Penicillamine
CMXRLRGX

SEQ ID NO: 11 - protein, 8 aa, Artificial Sequence
  Chemically synthesized
  misc_feature (1)..(8): Cys and Cys form a ring
CMPRLRGC

SEQ ID NO: 12 - protein, 12 aa, Artificial Sequence
  Chemically synthesized
  misc_feature (4)..(9): Cys and Cys form a ring
HLDCMPRGCFRN

SEQ ID NO: 13 - protein, 9 aa, Artificial Sequence
  Chemically synthesized
  misc_feature (1)..(9): Cys and Cys form a ring
CQVKSMPRC

SEQ ID NO: 14 - protein, 9 aa, Artificial Sequence
  Chemically synthesized
  misc_feature (1)..(9): Cys and Cys form a ring
CTTPMPRLC

SEQ ID NO: 15 - protein, 9 aa, Artificial Sequence
  Chemically synthesized
  misc_feature (1)..(9): Cys and Cys form a ring
CKAPQMPRC

SEQ ID NO: 16 - protein, 9 aa, Artificial Sequence
  Chemically synthesized
  misc_feature (1)..(9): Cys and Cys form a ring
CLNPSMPRC

SEQ ID NO: 17 - protein, 9 aa, Artificial Sequence
  Chemically synthesized
  misc_feature (1)..(9): Cys and Cys form a ring
CLVSSMPRC

SEQ ID NO: 18 - protein, 9 aa, Artificial Sequence
  Chemically synthesized
  misc_feature (1)..(9): Cys and Cys form a ring
CLQPMPRLC

SEQ ID NO: 19 - protein, 9 aa, Artificial Sequence
  Chemically synthesized
  misc_feature (1)..(9): Cys and Cys form a ring
CPVSSMPRC

SEQ ID NO: 20 - protein, 9 aa, Artificial Sequence
  Chemically synthesized
  misc_feature (1)..(9): Cys and Cys form a ring
CQSPMPRLC

SEQ ID NO: 21 - protein, 9 aa, Artificial Sequence
  Chemically synthesized
  misc_feature (1)..(9): Cys and Cys form a ring
CLTPMPRLC

SEQ ID NO: 22 - protein, 15 aa, Artificial Sequence
  Chemically synthesized
  misc_feature (5)..(12): Cys and Cys form a ring
DSGLCMPRLRGCDPR

SEQ ID NO: 23 - protein, 15 aa, Artificial Sequence
  Chemically synthesized
TPSAHAMALQSLSVG

SEQ ID NO: 24 - protein, 15 aa, Artificial Sequence
  Chemically synthesized
  misc_feature (1)..(1): N-acetylated
  misc_feature (5)..(12): Cys and Cys form a ring
  misc_feature (15)..(15): terminus is C(=O)NH2
DSGLCMPRLRGCDPR

SEQ ID NO: 25 - protein, 8 aa, Artificial Sequence
  Chemically synthesized
  misc_feature (1)..(8): Cys and Cys form a ring
  misc_feature (1)..(1): N-propionylated
  misc_feature (8)..(8): terminus is C(=O)NH2
CMPRLRGC

SEQ ID NO: 26 - protein, 8 aa, Artificial Sequence
  Chemically synthesized
  misc_feature (1)..(8): Cys anc Cys form a ring
  misc_feature (1)..(1): N-propionylated D-amino acid
  misc_feature (8)..(8): terminus is C(=O)NH2
CMPRLRGC

SEQ ID NO: 27 - protein, 8 aa, Artificial Sequence
  Chemically synthesized
  misc_feature (1)..(1): N-propionylated D-amino acid
  misc_feature (1)..(8): Cys and Pen form a ring
  misc_feature (3)..(3): Xaa = thiazolidine-4-carboxylic acid
  misc_feature (8)..(8): Xaa = Penicillamine, terminus is C(=O)NH2
CMXRLRGX

SEQ ID NO: 28 - protein, 7 aa, Artificial Sequence
  Chemically synthesized
  misc_feature (1)..(7): Cys and Cys form a ring
  misc_feature (1)..(1): N-acetylated
  misc_feature (7)..(7): terminus is C(=O)NH2
CMPRLGC

SEQ ID NO: 29 - protein, 8 aa, Artificial Sequence
  Chemically synthesized
  misc_feature (1)..(8): Cys and Cys form a ring
  misc_feature (1)..(1): N-acetylated D-amino acid
  misc_feature (8)..(8): terminus is C(=O)NH2
CMPRLRGC

SEQ ID NO: 30 - protein, 9 aa, Artificial Sequence
  Chemically synthesized
  misc_feature (1)..(1): N-acetylated
  misc_feature (2)..(9): Pen and Cys form a ring
  misc_feature (2)..(2): Xaa = Penicillamine
  misc_feature (4)..(4): Xaa = thiazolidine-4-carboxylic acid
  misc_feature (9)..(9): terminus is C(=O)NH2
DXMXRLRGC

SEQ ID NO: 31 - protein, 8 aa, Artificial Sequence
  Chemically synthesized
  misc_feature (1)..(8): Cys and Pen form a ring
  misc_feature (1)..(1): N-propionylated D-amino acid
  misc_feature (3)..(3): Xaa = thiazolidine-4-carboxylic acid
  misc_feature (8)..(8): Xaa = Penicillamine
  misc_feature (8)..(8): terminus is C(=O)NH2
CMXRLRGX

SEQ ID NO: 32 - protein, 8 aa, Artificial Sequence
  Chemically synthesized
  misc_feature (1)..(8): Cys and Pen form a ring
  misc_feature (1)..(1): N-propionylated D-amino acid
  misc_feature (3)..(3): Xaa = thiazolidine-4-carboxylic acid
  misc_feature (7)..(7): Xaa = sarcosine
  misc_feature (8)..(8): Xaa = Penicillamine
  misc_feature (8)..(8): terminus is C(=O)NH2
CMXRLRXX

SEQ ID NO: 33 - protein, 8 aa, Artificial Sequence
  Chemically synthesized
  misc_feature (1)..(8): Cys and Cys form a ring
  misc_feature (1)..(1): N-propionylated D-amino acid
  misc_feature (3)..(3): Xaa = pipecolic acid
  misc_feature (7)..(7): Xaa = sarcosine
  misc_feature (8)..(8): terminus is C(=O)NH2
CMXRLRXC

SEQ ID NO: 34 - protein, 8 aa, Artificial Sequence
  Chemically synthesized
  misc_feature (1)..(8): Cys and Pen form a ring
  misc_feature (1)..(1): N-propionylated D-amino acid
  misc_feature (3)..(3): Xaa = pipecolic acid
  misc_feature (8)..(8): Xaa = Penicillamine
  misc_feature (8)..(8): terminus is C(=O)NH2
CMXRLRGX

SEQ ID NO: 35 - protein, 8 aa, Artificial Sequence
  Chemically synthesized
  misc_feature (1)..(8): Cys and Pen form a ring
  misc_feature (1)..(1): N-propionylated D-amino acid
  misc_feature (3)..(3): Xaa = pipecolic acid
  misc_feature (7)..(7): Xaa = sarcosine
  misc_feature (8)..(8): Xaa = Penicillamine
  misc_feature (8)..(8): terminus is C(=O)NH2
CMXRLRXX

SEQ ID NO: 36 - protein, 16 aa, Artificial Sequence
  Chemically synthesized
  misc_feature (4)..(13): Cys and Cys form a ring
TDTCLMLPLLLGCDEE

SEQ ID NO: 37 - protein, 16 aa, Artificial Sequence
  Chemically synthesized
  misc_feature (4)..(13): Cys and Cys form a ring
DPICWYFPRLLGCTTL

SEQ ID NO: 38 - protein, 16 aa, Artificial Sequence
  Chemically synthesized
  misc_feature (4)..(13): Cys and Cys form a ring
WYPCYIYPRLLGCDGD

SEQ ID NO: 39 - protein, 16 aa, Artificial Sequence
  Chemically synthesized
  misc_feature (4)..(13): Cys and Cys form a ring
GNICMLIPGLLGCSYE

SEQ ID NO: 40 - protein, 16 aa, Artificial Sequence
  Chemically synthesized
  misc_feature (4)..(13): Cys and Cys form a ring
VNSCLLLPNLLGCGDD

SEQ ID NO: 41 - protein, 16 aa, Artificial Sequence
  Chemically synthesized
TPVCILLPSLLGCDTQ

SEQ ID NO: 42 - protein, 16 aa, Artificial Sequence
  Chemically synthesized
  misc_feature (4)..(13): Cys and Cys form a ring
TVLCSLWPELLGCPPE

SEQ ID NO: 43 - protein, 16 aa, Artificial Sequence
  Chemically synthesized
  misc_feature (4)..(13): Cys and Cys form a ring
TFSCLMWPWLLGCESL

SEQ ID NO: 44 - protein, 16 aa, Artificial Sequence
  Chemically synthesized
  misc_feature (4)..(13): Cys and Cys form a ring
FGTCYTWPWLLGCEGF

SEQ ID NO: 45 - protein, 16 aa, Artificial Sequence
  Chemically synthesized
  misc_feature (4)..(13): Cys and Cys form a ring
SLFCRLLLTPVGCVSQ

SEQ ID NO: 46 - protein, 16 aa, Artificial Sequence
  Chemically synthesized
HLLVLPRGLLGCTTLA

SEQ ID NO: 47 - protein, 16 aa, Artificial Sequence
  Chemically synthesized
  misc_feature (4)..(13): Cys and Cys form a ring
TSLCSMFPDLLGCFNL

SEQ ID NO: 48 - protein, 16 aa, Artificial Sequence
  Chemically synthesized
  misc_feature (4)..(13): Cys and Cys form a ring
SHPCGRLPMLLGCAES

SEQ ID NO: 49 - protein, 17 aa, Artificial Sequence
  Chemically synthesized
TSTCSMVPGPLGAVSTW

SEQ ID NO: 50 - protein, 16 aa, Artificial Sequence
  Chemically synthesized
  misc_feature (4)..(13): Cys and Cys form a ring
KDPCTRWAMLLGCDGE

SEQ ID NO: 51 - protein, 16 aa, Artificial Sequence
  Chemically synthesized
  misc_feature (4)..(13): Cys and Cys form a ring
IMTCSVYPFLLGCVDK

SEQ ID NO: 52 - protein, 16 aa, Artificial Sequence
  Chemically synthesized
  misc_feature (4)..(13): Cys and Cys form a ring
IHSCAHVMRLLGCWSR

SEQ ID NO: 53 - protein, 10 aa, Artificial Sequence
  Chemically synthesized
CGGGPFWWWP

SEQ ID NO: 54 - protein, 10 aa, Artificial Sequence
  Chemically synthesized
CGGGHKYLRW

SEQ ID NO: 55 - protein, 10 aa, Artificial Sequence
  Chemically synthesized
CGGGKRIFMV

SEQ ID NO: 56 - protein, 10 aa, Artificial Sequence
  Chemically synthesized
CGGGKWHYLR

SEQ ID NO: 57 - protein, 12 aa, Artificial Sequence
  Chemically synthesized
THRPPMWSPVWP

SEQ ID NO: 58 - protein, 12 aa, Artificial Sequence
  Chemically synthesized
THRPPMWSPVWP

SEQ ID NO: 59 - protein, 12 aa, Artificial Sequence
  Chemically synthesized
THRPPMWSPVWP

SEQ ID NO: 60 - protein, 15 aa, Artificial Sequence
  Chemically synthesized
LSLERFLRCWSDAPA

SEQ ID NO: 61 - protein, 13 aa, Artificial Sequence
  Chemically synthesized
LERFLRCWSDAPA

SEQ ID NO: 62 - protein, 11 aa, Artificial Sequence
  Chemically synthesized
RFLRCWSDAPA

SEQ ID NO: 63 - protein, 9 aa, Artificial Sequence
  Chemically synthesized
LRCWSDAPA

SEQ ID NO: 64 - protein, 7 aa, Artificial Sequence
  Chemically synthesized
CWSDAPA

SEQ ID NO: 65 - protein, 18 aa, Artificial Sequence
  Chemically synthesized
DWFKAFYDKVAEKFKEAF

SEQ ID NO: 66 - protein, 13 aa, Artificial Sequence
  Chemically synthesized
  misc_feature (13)..(13): terminus is C(=O)OH or C(=O)NH2
TVFTSWEEYLDWV

SEQ ID NO: 67 - protein, 19 aa, Artificial Sequence
  Chemically synthesized
  misc_feature (19)..(19): terminus is C(=O)OH or C(=O)NH2
VEPNCDIHVMWEWECFERL

SEQ ID NO: 68 - protein, 9 aa, Artificial Sequence
  Chemically synthesized
  misc_feature (9)..(9): terminus is C(=O)OH or C(=O)NH2
KRFKQDGGC

SEQ ID NO: 69 - protein, 10 aa, Artificial Sequence
  Chemically synthesized
  misc_feature (10)..(10): terminus is C(=O)OH or C(=O)NH2
RGQILSKLRL

SEQ ID NO: 70 - protein, 17 aa, Artificial Sequence
  Chemically synthesized
STFCLLGQKDQSYCFTI

SEQ ID NO: 71 - protein, 16 aa, Artificial Sequence
  Chemically synthesized
HMRCLHYKGRRVCFLL

SEQ ID NO: 72 - protein, 16 aa, Artificial Sequence
  Chemically synthesized
KTMCLRYNHDKVCFRI

SEQ ID NO: 73 - protein, 18 aa, Artificial Sequence
  Chemically synthesized
LVLCLVHRTSKHRKCFVI

SEQ ID NO: 74 - protein, 7 aa, Artificial Sequence
  Chemically synthesized
HAIYPRH

SEQ ID NO: 75 - protein, 16 aa, Artificial Sequence
  Chemically synthesized
SDVCLRYRGRPVCFQV

SEQ ID NO: 76 - protein, 16 aa, Artificial Sequence
  Chemically synthesized
HMVCLAYRGRPVCFAL

SEQ ID NO: 77 - protein, 16 aa, Artificial Sequence
  Chemically synthesized
HMVCLSYRGRPVCFSL

SEQ ID NO: 78 - protein, 16 aa, Artificial Sequence
  Chemically synthesized
HQVCLSYRGRPVCFST

SEQ ID NO: 79 - protein, 16 aa, Artificial Sequence
  Chemically synthesized
RDVCLRYRGRPVCFQV

SEQ ID NO: 80 - protein, 16 aa, Artificial Sequence
  Chemically synthesized
HDVCLRYRGRPVCFQV

SEQ ID NO: 81 - protein, 16 aa, Artificial Sequence
  Chemically synthesized
ADVCLRYRGRPVCFQV

SEQ ID NO: 82 - protein, 16 aa, Artificial Sequence
  Chemically synthesized
SAVCLRYRGRPVCFQV

SEQ ID NO: 83 - protein, 16 aa, Artificial Sequence
  Chemically synthesized
SMVCLRYRGRPVCFQV

SEQ ID NO: 84 - protein, 16 aa, Artificial Sequence
  Chemically synthesized
SDRCLRYRGRPVCFQV

SEQ ID NO: 85 - protein, 16 aa, Artificial Sequence
  Chemically synthesized
SDACLRYRGRPVCFQV

SEQ ID NO: 86 - protein, 16 aa, Artificial Sequence
  Chemically synthesized
SDVCARYRGRPVCFQV

SEQ ID NO: 87 - protein, 16 aa, Artificial Sequence
  Chemically synthesized
SDVCLNYRGRPVCFQV

SEQ ID NO: 88 - protein, 16 aa, Artificial Sequence
  Chemically synthesized
SDVCLHYRGRPVCFQV

SEQ ID NO: 89 - protein, 16 aa, Artificial Sequence
  Chemically synthesized
SDVCLAYRGRPVCFQV

SEQ ID NO: 90 - protein, 16 aa, Artificial Sequence
  Chemically synthesized
SDVCLRARGRPVCFQV

SEQ ID NO: 91 - protein, 16 aa, Artificial Sequence
  Chemically synthesized
SDVCLRYAGRPVCFQV

SEQ ID NO: 92 - protein, 16 aa, Artificial Sequence
  Chemically synthesized
SDVCLRYRARPVCFQV

SEQ ID NO: 93 - protein, 16 aa, Artificial Sequence
  Chemically synthesized
SDVCLRYRGSPVCFQV

SEQ ID NO: 94 - protein, 16 aa, Artificial Sequence
  Chemically synthesized
SDVCLRYRGAPVCFQV

SEQ ID NO: 95 - protein, 16 aa, Artificial Sequence
  Chemically synthesized
SDVCLRYRGRRVCFQV

SEQ ID NO: 96 - protein, 16 aa, Artificial Sequence
  Chemically synthesized
SDVCLRYRGRAVCFQV

SEQ ID NO: 97 - protein, 16 aa, Artificial Sequence
  Chemically synthesized
SDVCLRYRGRPACFQV

SEQ ID NO: 98 - protein, 16 aa, Artificial Sequence
  Chemically synthesized
SDVCLRYRGRPVCRQV

SEQ ID NO: 99 - protein, 16 aa, Artificial Sequence
  Chemically synthesized
SDVCLRYRGRPVCAQV

SEQ ID NO: 100 - protein, 16 aa, Artificial Sequence
  Chemically synthesized
SDVCLRYRGRPVCFRV

SEQ ID NO: 101 - protein, 16 aa, Artificial Sequence
  Chemically synthesized
SDVCLRYRGRPVCFLV

SEQ ID NO: 102 - protein, 16 aa, Artificial Sequence
  Chemically synthesized
SDVCLRYRGRPVCFAV

SEQ ID NO: 103 - protein, 16 aa, Artificial Sequence
  Chemically synthesized
SDVCLRYRGRPVCFQW

SEQ ID NO: 104 - protein, 16 aa, Artificial Sequence
  Chemically synthesized
SDVCLRYRGRPVCFQL

SEQ ID NO: 105 - protein, 16 aa, Artificial Sequence
  Chemically synthesized
SDVCLRYRGRPVCFQA

SEQ ID NO: 106 - protein, 18 aa, Artificial Sequence
  Chemically synthesized
GRYQCQYRIGHYRFRYSD

SEQ ID NO: 107 - protein, 18 aa, Artificial Sequence
  Chemically synthesized
GRYQAQYRIGHYRFRYSD

SEQ ID NO: 108 - protein, 18 aa, Artificial Sequence
  Chemically synthesized
GRYQCQYRIGHYRFRYSD

SEQ ID NO: 109 - protein, 7 aa, Artificial Sequence
  Chemically synthesized
  misc_feature (1)..(7): Cys and Cys bond to a “CLIPS” cross-linker to form a ring (see p. 81 of the as-filed specification)
CHYRFRC

SEQ ID NO: 110 - protein, 10 aa, Artificial Sequence
  Chemically synthesized
  misc_feature (1)..(10): Cys and Cys bond to a “CLIPS” cross-linker to form a ring (see p. 81 of the as-filed specification)
CRIGHYRFRC

SEQ ID NO: 111 - protein, 10 aa, Artificial Sequence
  Chemically synthesized
  misc_feature (4)..(10): Cys and Cys bond to a “CLIPS” cross-linker to form a ring (see p. 81 of the as-filed specification)
YQACHYRFRC

SEQ ID NO: 112 - protein, 14 aa, Artificial Sequence
  Chemically synthesized
  misc_feature (6)..(14): Cys and Cys bond to a “CLIPS” cross-linker to form a ring (see p. 81 of the as-filed specification)
RYQAQCRIGHYRFC

SEQ ID NO: 113 - protein, 18 aa, Artificial Sequence
  Chemically synthesized
  misc_feature (5)..(17): Cys and Cys bond to a “CLIPS” cross-linker to form a ring (see p. 81 of the as-filed specification)
GRYQCQYRIGHYRFRYCD

SEQ ID NO: 114 - protein, 18 aa, Artificial Sequence
  Chemically synthesized
  misc_feature (6)..(16): Cys and Cys bond to a “CLIPS” cross-linker to form a ring (see p. 81 of the as-filed specification)
GRYQACYRIGHYRFRCSD

SEQ ID NO: 115 - protein, 18 aa, Artificial Sequence
  Chemically synthesized
  misc_feature (7)..(15): Cys and Cys bond to a “CLIPS” cross-linker to form a ring (see p. 81 of the as-filed specification)
GRYQAQCRIGHYRFCYSD

SEQ ID NO: 116 - protein, 14 aa, Artificial Sequence
  Chemically synthesized
RYQAQCRIGHYRFC

SEQ ID NO: 117 - protein, 18 aa, Artificial Sequence
  Chemically synthesized
GRYQCQYRIGHYRFRYCD

SEQ ID NO: 118 - protein, 18 aa, Artificial Sequence
  Chemically synthesized
GRYQACYRIGHYRFRCSD

SEQ ID NO: 119 - protein, 18 aa, Artificial Sequence
  Chemically synthesized
GRYQAQCRIGHYRFCYSD

SEQ ID NO: 120 - protein, 7 aa, Artificial Sequence
  Chemically synthesized
STPTRYS

SEQ ID NO: 121 - protein, 9 aa, Artificial Sequence
  Chemically synthesized
CALWHWWHC

SEQ ID NO: 122 - protein, 9 aa, Artificial Sequence
  Chemically synthesized
  misc_feature (2)..(2): Xaa = Thr or Ser
CXWLHWWAC

SEQ ID NO: 123 - protein, 8 aa, Artificial Sequence
  Chemically synthesized
  misc_feature (1)..(1): Xaa = Leu or Met
  misc_feature (5)..(5): Xaa = Tyr or Phe
  misc_feature (6)..(6): Xaa = Leu or Met
  misc_feature (7)..(7): Xaa = any natural amino acid
  misc_feature (8)..(8): Xaa = Trp, Tyr, or Phe
XHELXXXX

SEQ ID NO: 124 - protein, 25 aa, Artificial Sequence
  Chemically synthesized
  misc_feature (1)..(25): All amino acids are D-configuration
DDDEKQLKERWYKRWLEYLDEFKKN

SEQ ID NO: 125 - protein, 25 aa, Artificial Sequence
  Chemically synthesized
  misc_feature (1)..(25): All amino acids are D-configuration
TEEEKQLKEWWYKHWQEYLEEFKKN

SEQ ID NO: 126 - protein, 20 aa, Artificial Sequence
  Chemically synthesized
GACPPCLWQVLCGGSGSGSG

SEQ ID NO: 127 - protein, 12 aa, Artificial Sequence
  Chemically synthesized
HIHDDLLRYYGW

SEQ ID NO: 128 - protein, 27 aa, Artificial Sequence
  Chemically synthesized
  misc_feature (13)..(13): NH2 of Lys is bonded to SEQ ID NO127
  misc_feature (14)..(14): C(=O)OH is amidated to C(=O)NH2
  misc_feature (15)..(15): NH2 of Lys is bonded to SEQ ID NO127
HIHDDLLRYYGWKKKWGYYRLLDDHIH

SEQ ID NO: 129 - protein, 7 aa, Artificial Sequence
  Chemically synthesized
KRWSRYF

SEQ ID NO: 130 - protein, 9 aa, Artificial Sequence
  Chemically synthesized
YCWSQYLCY

SEQ ID NO: 131 - protein, 15 aa, Artificial Sequence
  Chemically synthesized
DFLPHYKNTSLGHRP

SEQ ID NO: 132 - protein, 9 aa, Artificial Sequence
  Chemically synthesized
YCLYQSWCY

SEQ ID NO: 133 - protein, 13 aa, Artificial Sequence
  Chemically synthesized
  misc_feature (2)..(12): Cys and Cys form a ring
TCWVLEGLHWACD

SEQ ID NO: 134 - protein, 15 aa, Artificial Sequence
  Chemically synthesized
  misc_feature (1)..(15): Cys and Cys form a ring
  misc_feature (3)..(13): Cys and Cys form a ring
  misc_feature (15)..(15): terminus is C(=O)NH2
CTCWVLEGLHWACDC

SEQ ID NO: 135 - protein, 13 aa, Artificial Sequence
  Chemically synthesized
TVFTSWEEYLDWV

SEQ ID NO: 136 - protein, 19 aa, Artificial Sequence
  Chemically synthesized
VEPNCDIHVMWEWECFERL

SEQ ID NO: 137 - protein, 10 aa, Artificial Sequence
  Chemically synthesized
KRFKQDDGGC

SEQ ID NO: 138 - protein, 10 aa, Artificial Sequence
  Chemically synthesized
RGQILSKLRL

SEQ ID NO: 139 - protein, 13 aa, Artificial Sequence
  Chemically synthesized
DCAWHLGELVWCT

SEQ ID NO: 140 - protein, 15 aa, Artificial Sequence
  Chemically synthesized
CDCAWHLGELVWCTC